(19) United States
(12) Patent Application Publication
(10) Pub. No.: US 2023/0247902 A1
(43) Pub. Date: Aug. 3, 2023

(54) HETEROCYCLIC COMPOUND, ORGANIC LIGHT-EMITTING DEVICE COMPRISING SAME, AND COMPOSITION FOR ORGANIC LAYER OF ORGANIC LIGHT-EMITTING DEVICE

(71) Applicant: LT MATERIALS CO., LTD., Yongin-si, Gyeonggi-do (KR)

(72) Inventors: Young-Seok NO, Yongin-si (KR); Geon-Yu PARK, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/011,934

(22) PCT Filed: Jul. 21, 2021
(86) PCT No.: PCT/KR2021/009383

§ 371 Date: Dec. 21, 2022

(30) Foreign Application Priority Data
Jul. 27, 2020 (KR) 10-2020-0093407

(51) Int. Cl. H10K 85/60; C07D 405/14; C07D 495/04; C07D 409/14; C07D 209/86

(52) U.S. Cl. CPC H10K 85/6572; C07D 405/14; H10K 85/654; H10K 85/6574; C07D 495/04; H10K 85/657; H10K 85/636; H10K 85/6576; C07D 409/14; C07D 209/86; H10K 50/11

(57) ABSTRACT

The present application provides a heterocyclic compound capable of significantly enhancing lifetime, efficiency, electrochemical stability and thermal stability of an organic light emitting device, an organic light emitting device comprising the heterocyclic compound in an organic material layer, and a composition for an organic material layer.

### TECHNICAL FIELD

This application claims priority to and the benefits of Korean Patent Application No. 10-2020-0093407, filed with the Korean Intellectual Property Office on Jul. 27, 2020, the entire contents of which are incorporated herein by reference.

The present specification relates to a heterocyclic compound, an organic light emitting device comprising the same, and a composition for an organic material layer of an organic light emitting device.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

The present disclosure is directed to providing a heterocyclic compound, and an organic light emitting device comprising the same.

### Technical Solution

One embodiment of the present application provides a heterocyclic compound represented by the following Chemical Formula 1.

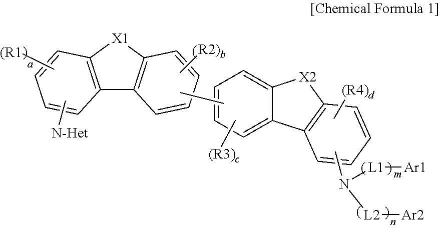

In Chemical Formula 1,

X1 and X2 are the same as or different from each other, and each independently O or S,

L1 and L2 are the same as or different from each other, and each independently a direct bond; a substituted or unsubstituted arylene group having 6 to 60 carbon atoms; or a substituted or unsubstituted heteroarylene group having 2 to 60 carbon atoms, and m and n are each independently 0 or 1,

Ar1 and Ar2 are the same as or different from each other, and each independently selected from the group consisting of a substituted or unsubstituted aryl group having 6 to 60 carbon atoms and a substituted or unsubstituted heteroaryl group having 2 to 60 carbon atoms, or two or more groups adjacent to each other bond to each other to form a substituted or unsubstituted aliphatic or aromatic hydrocarbon ring or heteroring,

R1 to R4 are the same as or different from each other, and each independently hydrogen; deuterium; a substituted or unsubstituted aryl group having 6 to 60 carbon atoms; or a substituted or unsubstituted heteroaryl group having 6 to 60 carbon atoms, a to d are each an integer of 0 to 3, and when a to d are each 2 or greater, substituents in the parentheses are the same as each other, and

N-Het is a monocyclic or polycyclic heterocyclic group having 1 to 60 carbon atoms substituted or unsubstituted, and comprising one or more Ns.

Another embodiment of the present application provides an organic light emitting device comprising a first electrode, a second electrode, and one or more organic material layers provided between the first electrode and the second electrode, wherein one or more layers of the organic material layers comprise the heterocyclic compound represented by Chemical Formula 1.

Another embodiment of the present application provides a composition for an organic material layer of an organic light emitting device, the composition comprising the heterocyclic compound represented by Chemical Formula 1 and a heterocyclic compound represented by the following Chemical Formula 13 or 14.

### Advantageous Effects

A heterocyclic compound according to one embodiment of the present application can be used as a material of an organic material layer of an organic light emitting device. The heterocyclic compound can be used as a material of a hole injection layer, a hole transfer layer, a light emitting layer, an electron transfer layer, an electron injection layer, a charge generation layer and the like in an organic light emitting device. Particularly, the heterocyclic compound represented by Chemical Formula 1 can be used as a material of a light emitting layer of an organic light emitting device. In addition, using the heterocyclic compound represented by Chemical Formula 1 in an organic light emitting device is capable of lowering a driving voltage of the device, enhancing light efficiency, and enhancing lifetime properties of the device by thermal stability of the compound.

### DESCRIPTION OF DRAWINGS

FIG. 1 to FIG. 3 are diagrams each schematically illustrating a lamination structure of an organic light emitting device according to one embodiment of the present application.

### MODE FOR DISCLOSURE

Hereinafter, the present application will be described in detail.

One embodiment of the present application provides a heterocyclic compound represented by the following Chemical Formula 1.

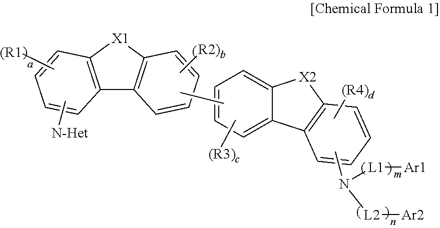

In Chemical Formula 1,

X1 and X2 are the same as or different from each other, and each independently 0 or S,

L1 and L2 are the same as or different from each other, and each independently a direct bond; a substituted or unsubstituted arylene group having 6 to 60 carbon atoms; or a substituted or unsubstituted heteroarylene group having 2 to 60 carbon atoms, and m and n are each independently 0 or 1,

Ar1 and Ar2 are the same as or different from each other, and each independently selected from the group consisting of a substituted or unsubstituted aryl group having 6 to 60 carbon atoms and a substituted or unsubstituted heteroaryl group having 2 to 60 carbon atoms, or two or more groups adjacent to each other bond to each other to form a substituted or unsubstituted aliphatic or aromatic hydrocarbon ring or heteroring,

R1 to R4 are the same as or different from each other, and each independently hydrogen; deuterium; a substituted or unsubstituted aryl group having 6 to 60 carbon atoms; or a substituted or unsubstituted heteroaryl group having 6 to 60 carbon atoms, a to d are each an integer of 0 to 3, and when a to d are each 2 or greater, substituents in the parentheses are the same as each other, and

N-Het is a monocyclic or polycyclic heterocyclic group having 1 to 60 carbon atoms substituted or unsubstituted, and comprising one or more Ns.

The compound represented by Chemical Formula 1 is substituted with carbazole or amine having a strong ability to push electrons and thereby has a high HOMO (highest occupied molecular orbital) level, and accordingly, is capable of helping with hole transfer when used as a material, particularly a dopant material, of a light emitting layer of an organic light emitting device.

In addition, the compound represented by Chemical Formula 1 has a structure in which LUMO (lowest unoccupied molecular orbital) is expanded and is thereby capable of enhancing electron mobility and stabilizing electrons, and as a result, device efficiency and lifetime may be enhanced when using the compound represented by Chemical Formula 1 in an organic light emitting device.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, the “substituted or unsubstituted” means being substituted with one or more substituents selected from the group consisting of deuterium; a cyano group; linear or branched alkyl having 1 to 60 carbon atoms; linear or branched alkenyl having 2 to 60 carbon atoms; linear or branched alkynyl having 2 to 60 carbon atoms; monocyclic or polycyclic cycloalkyl having 3 to 60 carbon atoms; monocyclic or polycyclic heterocycloalkyl having 2 to 60 carbon atoms; monocyclic or polycyclic aryl having 6 to 60 carbon atoms; monocyclic or polycyclic heteroaryl having 2 to 60 carbon atoms; —SiRR′R″; —P(═O)RR′; alkylamine having 1 to 20 carbon atoms; monocyclic or polycyclic arylamine having 6 to 60 carbon atoms; and monocyclic or polycyclic heteroarylamine having 2 to 60 carbon atoms, or being unsubstituted, or being substituted with a substituent linking two or more substituents selected from among the substituents illustrated above, or being unsubstituted, and R, R′ and R″ are the same as or different from each other and each independently substituted or unsubstituted alkyl having 1 to 60 carbon atoms; substituted or unsubstituted aryl having 6 to 60 carbon atoms; or substituted or unsubstituted heteroaryl having 2 to 60 carbon atoms.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0%, a hydrogen content being 100% or substituents being all hydrogen.

In one embodiment of the present application, deuterium is one of isotopes of hydrogen, is an element having deuteron formed with one proton and one neutron as a nucleus, and may be expressed as hydrogen-2, and the elemental symbol may also be written as D or ²H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

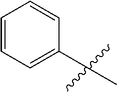 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

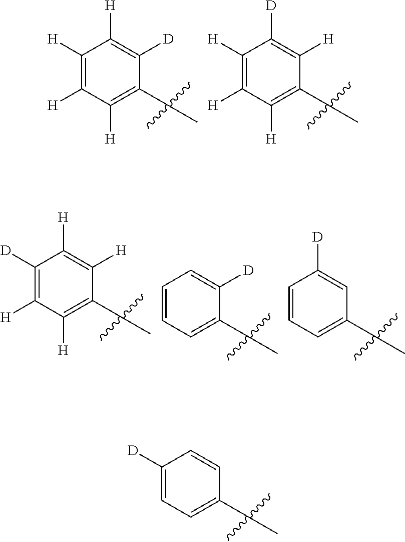

In addition, in one embodiment of the present application, “a phenyl group having a deuterium content of 0%” may mean a phenyl group that does not include a deuterium atom, that is, a phenyl group that has 5 hydrogen atoms.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide group may include a diphenylphosphine oxide group, a dinaphthylphosphine oxide group and the like, but are not limited thereto.

In the present specification, the silyl group is a substituent including Si, having the Si atom directly linked as a radical, and is represented by —SiR104R105R106. R104 to R106 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

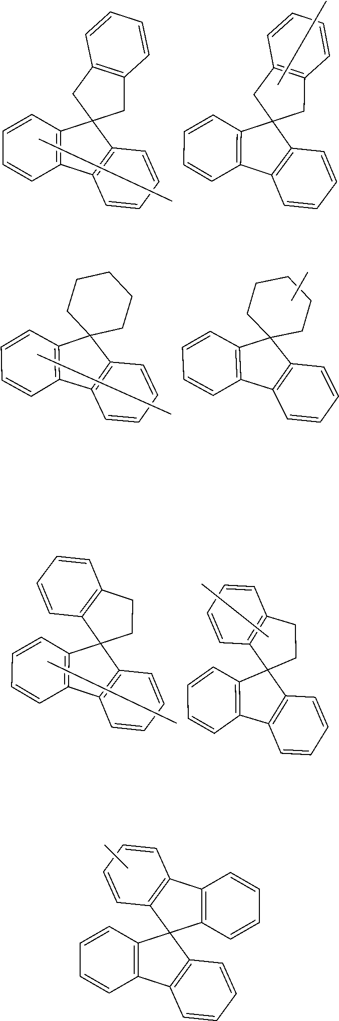

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto. In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except that these are each a divalent group. In addition, the heteroarylene group means the heteroaryl group having two bonding sites, that is, a divalent group. The descriptions on the heteroaryl group provided above may be applied thereto except that these are each a divalent group.

In the present specification, the “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

The heterocyclic compound according to one embodiment of the present application is represented by Chemical Formula 1. More specifically, by having a core structure and structural properties of the substituents as above, the heterocyclic compound represented by Chemical Formula 1 may be used as a material of an organic material layer of an organic light emitting device.

In one embodiment of the present application, the heterocyclic compound represented by Chemical Formula 1 may have a deuterium content of 0% to 100%.

In one embodiment of the present application, the heterocyclic compound represented by Chemical Formula 1 may have a deuterium content of greater than 10% and less than or equal to 100%.

In one embodiment of the present application, X1 and X2 of Chemical Formula 1 are the same as or different from each other, and may be each independently 0; or S.

In another embodiment, X1 and X2 are 0.

In another embodiment, X1 and X2 are S.

In another embodiment, X1 is 0 and X2 is S in Chemical Formula 1.

In another embodiment, X1 is S and X2 is O in Chemical Formula 1.

In one embodiment of the present application, L1 and L2 of Chemical Formula 1 are the same as or different from each other, and may be each independently a direct bond; a substituted or unsubstituted arylene group having 6 to 60 carbon atoms; or a substituted or unsubstituted heteroarylene group having 2 to 60 carbon atoms.

In one embodiment of the present application, L1 and L2 are the same as or different from each other, and may be each independently a direct bond; a substituted or unsubstituted arylene group having 6 to 40 carbon atoms; or a substituted or unsubstituted heteroarylene group having 2 to 40 carbon atoms.

In one embodiment of the present application, L1 and L2 are the same as or different from each other, and may be each independently a direct bond; a substituted or unsubstituted arylene group having 6 to 20 carbon atoms; or a substituted or unsubstituted heteroarylene group having 2 to 20 carbon atoms.

In one embodiment of the present application, L1 may be a direct bond; a substituted or unsubstituted arylene group having 6 to 60 carbon atoms; or a substituted or unsubstituted heteroarylene group having 2 to 60 carbon atoms.

In another embodiment, L1 is a direct bond.

In another embodiment, L1 is a phenylene group.

In one embodiment of the present application, L2 may be a direct bond; a substituted or unsubstituted arylene group having 6 to 60 carbon atoms; or a substituted or unsubstituted heteroarylene group having 2 to 60 carbon atoms.

In another embodiment, L2 is a direct bond.

In another embodiment, L2 is a phenylene group.

In one embodiment of the present application, Ar1 and Ar2 of Chemical Formula 1 are the same as or different from each other, and each independently selected from the group consisting of a substituted or unsubstituted aryl group having 6 to 60 carbon atoms and a substituted or unsubstituted heteroaryl group having 2 to 60 carbon atoms, or two or more groups adjacent to each other may bond to each other to form a substituted or unsubstituted aliphatic or aromatic hydrocarbon ring or heteroring.

In one embodiment of the present application, Ar1 and Ar2 are the same as or different from each other, and each independently selected from the group consisting of a substituted or unsubstituted aryl group having 6 to 40 carbon atoms and a substituted or unsubstituted heteroaryl group having 2 to 40 carbon atoms, or two or more groups adjacent to each other may bond to each other to form a substituted or unsubstituted aliphatic or aromatic hydrocarbon ring or heteroring.

In one embodiment of the present application, Ar1 and Ar2 are the same as or different from each other, and each independently selected from the group consisting of a substituted or unsubstituted aryl group having 6 to 20 carbon atoms and a substituted or unsubstituted heteroaryl group having 2 to 20 carbon atoms, or two or more groups adjacent to each other may bond to each other to form a substituted or unsubstituted aliphatic or aromatic hydrocarbon ring or heteroring.

In one embodiment of the present application, R1 to R4 of Chemical Formula 1 are the same as or different from each other, and may be each independently hydrogen; deuterium; a substituted or unsubstituted aryl group having 6 to 60 carbon atoms; or a substituted or unsubstituted heteroaryl group having 6 to 60 carbon atoms.

In another embodiment, R1 to R4 are hydrogen; or deuterium.

In another embodiment, R1 to R4 are hydrogen.

In another embodiment, R1 to R4 are deuterium.

In one embodiment of the present application, a to d of Chemical Formula 1 are each an integer of 0 to 3, and when a to d are each 2 or greater, substituents in the parentheses may be the same as each other.

In another embodiment, a is 0.

In another embodiment, a is 1.

In another embodiment, a is 2.

In another embodiment, a is 3.

In one embodiment of the present application, when a is 2 or greater, substituents in the parentheses are the same as each other.

In another embodiment, b is 0.

In another embodiment, b is 1.

In another embodiment, b is 2.

In another embodiment, b is 3.

In one embodiment of the present application, when b is 2 or greater, substituents in the parentheses are the same as each other.

In another embodiment, c is 0.

In another embodiment, c is 1.

In another embodiment, c is 2.

In another embodiment, c is 3.

In one embodiment of the present application, when c is 2 or greater, substituents in the parentheses are the same as each other.

In another embodiment, d is 0.

In another embodiment, d is 1.

In another embodiment, d is 2.

In another embodiment, d is 3.

In one embodiment of the present application, when d is 2 or greater, substituents in the parentheses are the same as each other.

In one embodiment of the present application, Chemical Formula 1 may be represented by any one of the following Chemical Formulae 1-1 to 1-4.

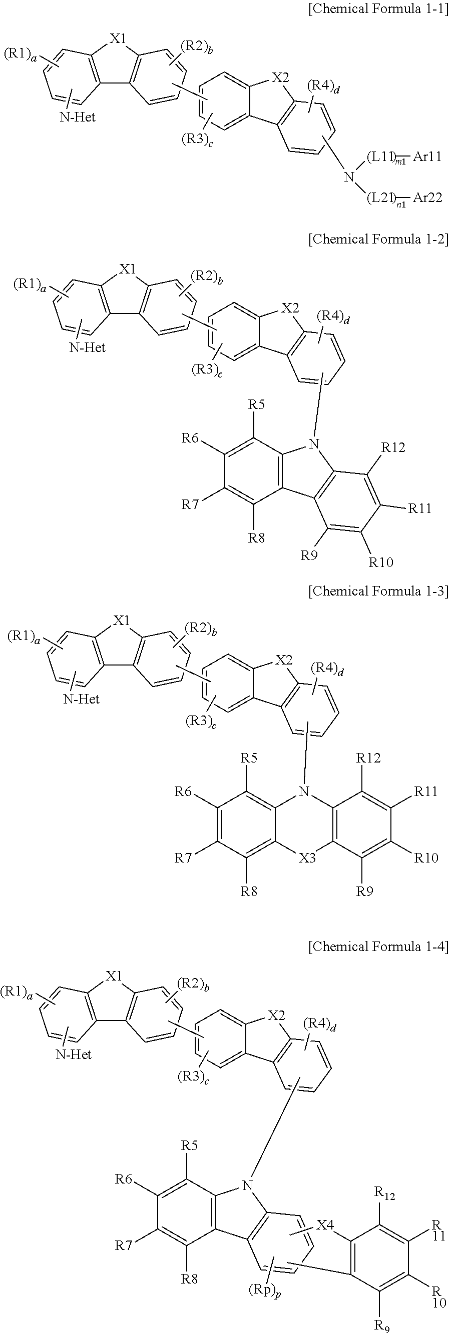

In Chemical Formulae 1-1 to 1-4,

L11 and L21 are the same as or different from each other, and each independently a direct bond; a substituted or unsubstituted arylene group having 6 to 60 carbon atoms; or a substituted or unsubstituted heteroarylene group having 2 to 60 carbon atoms, and m1 and n1 are each independently 0 or 1,

Ar11 and Ar21 are the same as or different from each other, and each independently a substituted or unsubstituted aryl group having 6 to 60 carbon atoms; or a substituted or unsubstituted heteroaryl group having 2 to 60 carbon atoms,

X3 and X4 are each independently O; S; CRaRb; or NRc, Ra and Rb are the same as or different from each other and each independently a substituted or unsubstituted alkyl group having 1 to 10 carbon atoms; or a substituted or unsubstituted aryl group having 6 to 20 carbon atoms, and Rc is a substituted or unsubstituted aryl group having 6 to 20 carbon atoms,

R5 to R12 and Rp are the same as or different from each other, and each independently hydrogen; deuterium; a halogen group; a cyano group; a substituted or unsubstituted aryl group having 6 to 20 carbon atoms; or a substituted or unsubstituted heteroaryl group having 2 to 20 carbon atoms, p is an integer of 0 to 2, and when p is 2, substituents in the parentheses are the same as each other, and

X1, X2, R1 to R4, N-Het and a to d have the same definitions as in Chemical Formula 1.

In one embodiment of the present application, L11 and L21 are the same as or different from each other, and may be each independently a direct bond; a substituted or unsubstituted arylene group having 6 to 40 carbon atoms; or a substituted or unsubstituted heteroarylene group having 2 to 40 carbon atoms.

In one embodiment of the present application, L11 and L21 are the same as or different from each other, and may be each independently a direct bond; a substituted or unsubstituted arylene group having 6 to 20 carbon atoms; or a substituted or unsubstituted heteroarylene group having 2 to 20 carbon atoms.

In one embodiment of the present application, L11 and L21 are the same as or different from each other, and may be each independently a direct bond; a substituted or unsubstituted phenylene group; a substituted or unsubstituted biphenylene group; or a substituted or unsubstituted naphthylene group.

In one embodiment of the present application, L11 and L21 are the same as or different from each other, and may be each independently a direct bond; a phenylene group; a biphenylene group; or a naphthylene group.

In one embodiment of the present application, L11 is a direct bond.

In one embodiment of the present application, L11 is a phenylene group.

In one embodiment of the present application, L11 is a biphenylene group.

In one embodiment of the present application, L11 is a naphthylene group.

In one embodiment of the present application, L21 is a direct bond.

In one embodiment of the present application, L21 is a phenylene group.

In one embodiment of the present application, L21 is a biphenylene group.

In one embodiment of the present application, L21 is a naphthylene group.

In one embodiment of the present application, Ar11 and Ar21 are the same as or different from each other, and may be each independently a substituted or unsubstituted aryl group having 6 to 60 carbon atoms; or a substituted or unsubstituted heteroaryl group having 2 to 60 carbon atoms.

In one embodiment of the present application, Ar11 and Ar21 are the same as or different from each other, and may be each independently a substituted or unsubstituted aryl group having 6 to 40 carbon atoms; or a substituted or unsubstituted heteroaryl group having 2 to 40 carbon atoms.

In one embodiment of the present application, Ar11 and Ar21 are the same as or different from each other, and may be each independently a substituted or unsubstituted aryl group having 6 to 20 carbon atoms; or a substituted or unsubstituted heteroaryl group having 2 to 20 carbon atoms.

In one embodiment of the present application, Ar11 and Ar21 are the same as or different from each other, and may be each independently a substituted or unsubstituted phenyl group; a substituted or unsubstituted biphenyl group; a substituted or unsubstituted naphthyl group; a substituted or unsubstituted fluorenyl group; a substituted or unsubstituted dibenzothiophene group; or a substituted or unsubstituted dibenzofuran group.

In one embodiment of the present application, Ar11 and Ar21 are the same as or different from each other, and may be each independently a substituted or unsubstituted phenyl group; a substituted or unsubstituted biphenyl group; a substituted or unsubstituted naphthyl group; a fluorenyl group unsubstituted or substituted with one or more selected from the group consisting of a phenyl group and an alkyl group having 1 to 10 carbon atoms; a substituted or unsubstituted dibenzothiophene group; or a substituted or unsubstituted dibenzofuran group.

In one embodiment of the present application, Ar11 and Ar21 are the same as or different from each other, and may be each independently a substituted or unsubstituted phenyl group; a substituted or unsubstituted biphenyl group; a substituted or unsubstituted naphthyl group; a fluorenyl group unsubstituted or substituted with one or more selected from the group consisting of a phenyl group and a methyl group; a substituted or unsubstituted dibenzothiophene group; or a substituted or unsubstituted dibenzofuran group.

In one embodiment of the present application, Ar11 and Ar21 are the same as or different from each other, and may be each independently a phenyl group; a biphenyl group; a naphthyl group; a fluorenyl group unsubstituted or substituted with one or more selected from the group consisting of a phenyl group and a methyl group; a substituted or unsubstituted dibenzothiophene group; or a substituted or unsubstituted dibenzofuran group.

In one embodiment of the present application, m1 and n1 may be each independently 0 or 1.

In one embodiment of the present application, R5 to R12 and Rp are the same as or different from each other, and each independently hydrogen; deuterium; a halogen group; a cyano group; a substituted or unsubstituted aryl group having 6 to 20 carbon atoms; or a substituted or unsubstituted heteroaryl group having 2 to 20 carbon atoms, p is an integer of 0 to 2, and when p is 2, substituents in the parentheses may be the same as each other.

In one embodiment of the present application, R5 to R12 and Rp are the same as or different from each other, and each independently hydrogen; deuterium; a halogen group; a cyano group; an aryl group having 6 to 20 carbon atoms unsubstituted or substituted with deuterium; or a heteroaryl group having 2 to 20 carbon atoms unsubstituted or substituted with deuterium, p is an integer of 0 to 2, and when p is 2, substituents in the parentheses may be the same as each other.

In one embodiment of the present application, R5 to R12 and Rp are the same as or different from each other, and each independently hydrogen; or deuterium, p is an integer of 0 to 2, and when p is 2, substituents in the parentheses may be the same as each other.

In one embodiment of the present application, R5 to R12 and Rp are hydrogen.

In one embodiment of the present application, R5 to R12 and Rp are deuterium.

In one embodiment of the present application, X3 and X4 are each independently O; S; CRaRb; or NRc, Ra and Rb are the same as or different from each other and may be each independently a substituted or unsubstituted alkyl group having 1 to 10 carbon atoms; or a substituted or unsubstituted aryl group having 6 to 20 carbon atoms, and Rc may be a substituted or unsubstituted aryl group having 6 to 20 carbon atoms.

In one embodiment of the present application, X3 and X4 are O; S; CRaRb; or NRc, and Rc may be a phenyl group.

In one embodiment of the present application, X3 is O.

In one embodiment of the present application, X3 is S.

In one embodiment of the present application, X3 is CRaRb, and Ra and Rb are the same as or different from each other and may be each independently a substituted or unsubstituted alkyl group having 1 to 10 carbon atoms; or a substituted or unsubstituted aryl group having 6 to 20 carbon atoms.

In one embodiment of the present application, Ra and Rb are the same as or different from each other, and each independently a substituted or unsubstituted methyl group; or a substituted or unsubstituted phenyl group.

In one embodiment of the present application, X3 is NRc, and Rc is a phenyl group.

In one embodiment of the present application, X4 is 0.

In one embodiment of the present application, X4 is S.

In one embodiment of the present application, X4 is CRaRb, and Ra and Rb are the same as or different from each other and may be each independently a substituted or unsubstituted alkyl group having 1 to 10 carbon atoms; or a substituted or unsubstituted aryl group having 6 to 20 carbon atoms.

In one embodiment of the present application, Ra and Rb are the same as or different from each other, and each independently a substituted or unsubstituted methyl group; or a substituted or unsubstituted phenyl group.

In one embodiment of the present application, X4 is NRc, and Rc is a phenyl group.

In one embodiment of the present application, N-Het is a monocyclic or polycyclic heteroring having 1 to 60 carbon atoms substituted or unsubstituted, and comprising one or more Ns.

In another embodiment, N-Het is a monocyclic or polycyclic heteroring having 1 to 60 carbon atoms unsubstituted or substituted with one or more substituents selected from the group consisting of an aryl group and a heteroaryl group, and comprising one or more Ns.

In another embodiment, N-Het is a monocyclic or polycyclic heteroring having 1 to 60 carbon atoms unsubstituted or substituted with one or more substituents selected from the group consisting of a phenyl group, a biphenyl group, a naphthyl group, a dimethylfluorene group, a dibenzofuran group, a diphenylfluorene group, a pyridine group, a spirobifluorene group and a dibenzothiophene group, and comprising one or more Ns.

In another embodiment, N-Het is a monocyclic or polycyclic heteroring unsubstituted or substituted with one or more substituents selected from the group consisting of a phenyl group, a biphenyl group, a naphthyl group, a dimethylfluorene group, a dibenzofuran group, a diphenylfluorene group, a pyridine group, a spirobifluorene group and a dibenzothiophene group, and comprising one or more and three or less Ns.

In one embodiment of the present application, N-Het is a monocyclic heteroring substituted or unsubstituted, and comprising one or more Ns.

In one embodiment of the present application, N-Het is a divalent or higher heteroring substituted or unsubstituted, and comprising one or more Ns.

In one embodiment of the present application, N-Het is a monocyclic or polycyclic heteroring substituted or unsubstituted, and comprising two or more Ns.

In one embodiment of the present application, N-Het is a divalent or higher polycyclic heteroring comprising two or more Ns.

In one embodiment of the present application, N-Het may be a pyrimidine group unsubstituted or substituted with a phenyl group; a triazine group unsubstituted or substituted with one or more substituents selected from the group consisting of a phenyl group, a biphenyl group, a naphthyl group, a dimethylfluorene group, a dibenzofuran group, a diphenylfluorene group, a pyridine group, a spirobifluorene group and a dibenzothiophene group; a benzimidazole group unsubstituted or substituted with a phenyl group; a quinazoline group unsubstituted or substituted with one or more substituents selected from the group consisting of a phenyl group and a biphenyl group; or a phenanthroline group unsubstituted or substituted with a phenyl group.

In one embodiment of the present application, N-Het may be a pyrimidine group unsubstituted or substituted with a phenyl group; a triazine group unsubstituted or substituted with one or more substituents selected from the group consisting of a phenyl group, a biphenyl group, a naphthyl group, a dimethylfluorene group, a dibenzofuran group, a diphenylfluorene group, a pyridine group, a spirobifluorene group and a dibenzothiophene group; a benzimidazole group unsubstituted or substituted with a phenyl group; a quinazoline group unsubstituted or substituted with one or more substituents selected from the group consisting of a phenyl group and a biphenyl group; or a phenanthroline group unsubstituted or substituted with a phenyl group.

In one embodiment of the present application, N-Het may be a group represented by any one of the following Chemical Formulae 2 to 4.

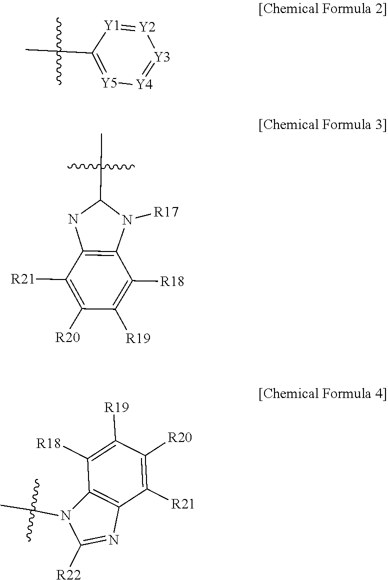

In Chemical Formulae 2 to 4,

Y1 is CR12 or N, Y2 is CR13 or N, Y3 is CR14 or N, Y4 is CR15 or N, Y5 is CR16 or N, and at least one of Y1 to Y5 is N,

R12 to R16 and R17 to R22 are the same as or different from each other, and each independently selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more groups adjacent to each other bond to each other to form a substituted or unsubstituted aliphatic or aromatic hydrocarbon ring or heteroring, and

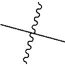

is a site linked to Chemical Formula 1.

In one embodiment of the present application, Chemical Formula 2 may be represented by one of the following Chemical Formulae 5 and 6. Herein,

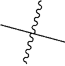

is a site linked to L of Chemical Formula 1.

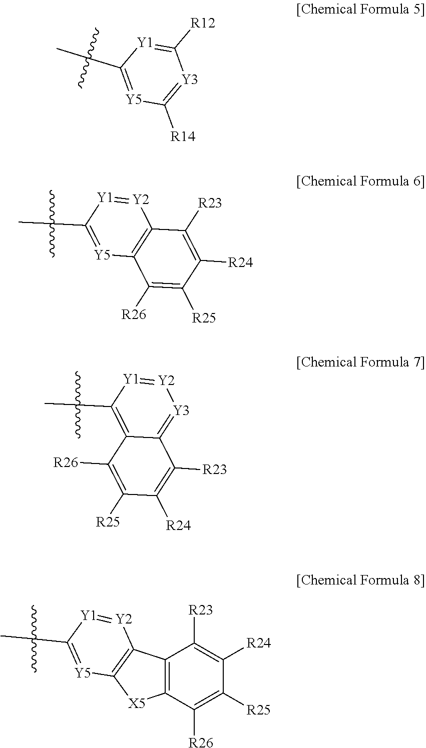

In Chemical Formula 5, one or more of Y1, Y3 and Y5 are N, and the rest have the same definitions as in Chemical Formula 5 and Chemical Formula 8,

in Chemical Formula 6, one or more of Y1, Y2 and Y5 are

N, and the rest have the same definitions as in Chemical Formula 5,

in Chemical Formula 7, one or more of Y1 to Y3 are N, and the rest have the same definitions as in Chemical Formula 5,

in Chemical Formula 8, one or more of Y1, Y2 and Y5 are N, and the rest have the same definitions as in Chemical Formula 5,

X5 is O; or S,

R12, R14 and R23 to R26 are the same as or different from each other, and each independently selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 6 to 60 carbon atoms; a substituted or unsubstituted phosphine oxide group; and a substituted or unsubstituted amine group, or two or more groups adjacent to each other bond to each other to form a substituted or unsubstituted aliphatic or aromatic hydrocarbon ring or heteroring.

In one embodiment of the present application, Chemical Formula 5 may be selected from among structural formulae of the following Group A.

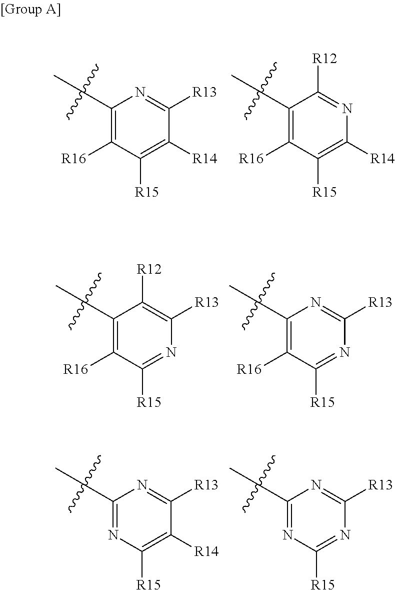

Substituents of the structural formulae of Group A have the same definitions as in Chemical Formula 5.

In one embodiment of the present application, Chemical Formula 6 may be represented by the following Chemical Formula 9.

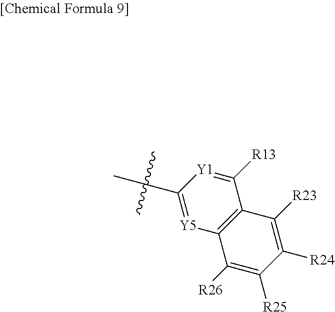

Substituents of Chemical Formula 9 have the same definitions as in Chemical Formula 6.

In one embodiment of the present application, Chemical Formula 7 may be represented by the following Chemical Formula 10.

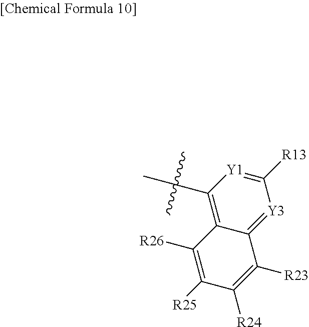

Substituents of Chemical Formula 10 have the same definitions as in Chemical Formula 7.

In one embodiment of the present application, Chemical Formula 6 may be represented by the following Chemical Formula 11.

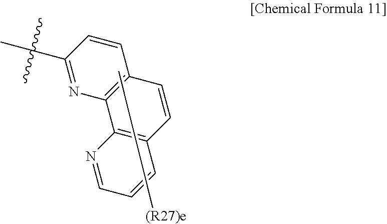

In Chemical Formula 11,

R27s are the same as or different from each other, and selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more groups adjacent to each other bond to each other to form a substituted or unsubstituted aliphatic or aromatic hydrocarbon ring or heteroring, e is an integer of 0 to 7, and when e is 2 or greater, R27s are the same as or different from each other.

In one embodiment of the present application, Chemical Formula 8 may be represented by the following Chemical Formula 12.

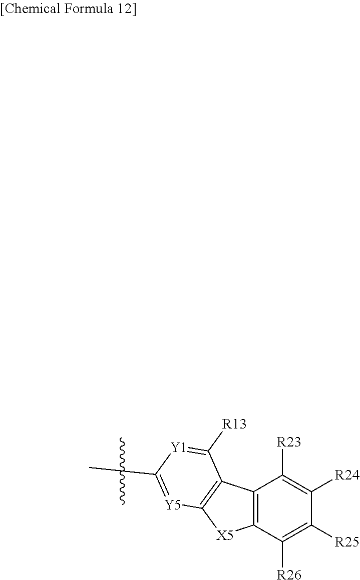

Substituents of Chemical Formula 12 have the same definitions as in Chemical Formula 8.

According to one embodiment of the present application, a bonding position where

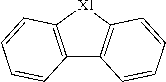

of Chemical Formula 1 bonds to N-Het of Chemical Formula 1 and

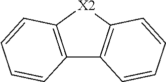

of Chemical Formula 1 is as shown in the following Group B.

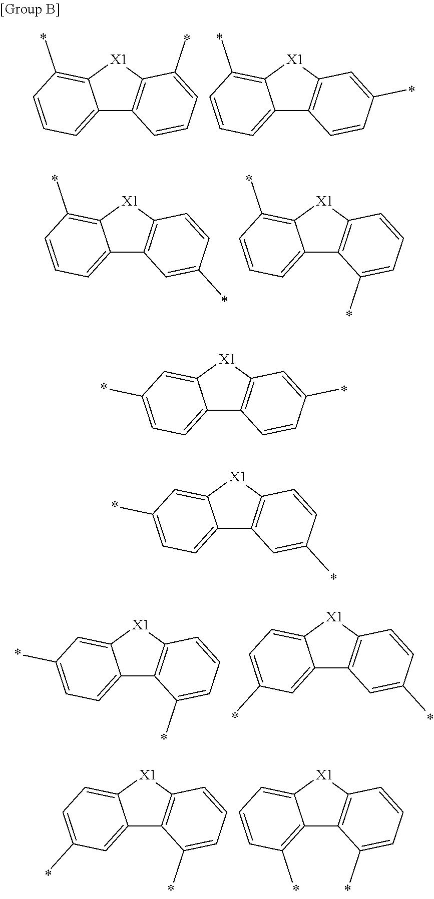

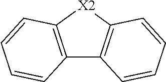

In Group B, * is a position where N-Het or bonds, and X₁, X₂ and N-Het have the same definitions as in Chemical Formula 1.

According to one embodiment of the present application, a bonding position where of

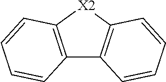

Chemical Formula 1 bonds to

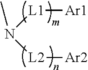

### of Chemical Formula 1 and

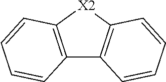

of Chemical Formula 1 is as shown in the following Group C.

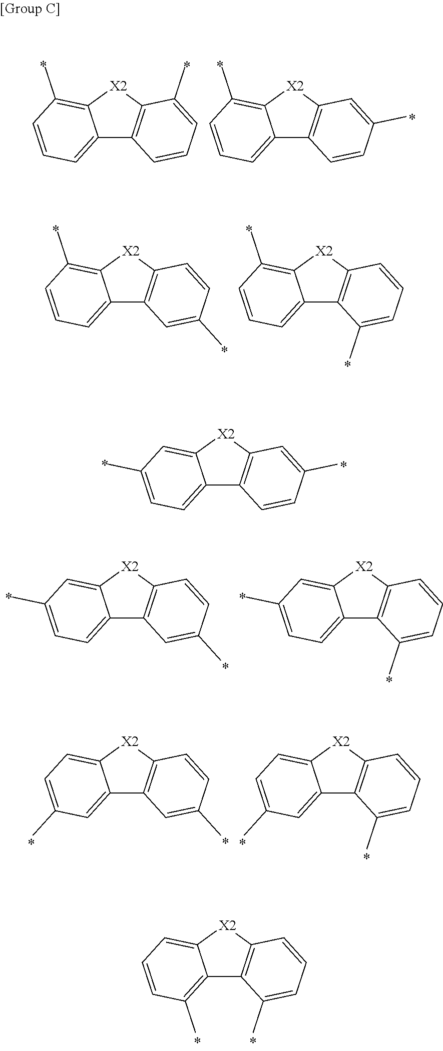

In Group C, * is a position where

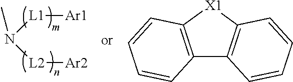

bonds, and X₁, X₂ and

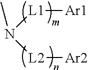

have the same definitions as in Chemical Formula 1.

More specifically, when a No. 3 position of

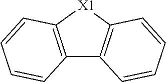

is substituted with N-Het and a No. 3 position of

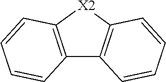

is substituted with

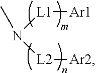

mobility and stability of holes and electrons may be further enhanced, and therefore, a lifetime of an organic light emitting device may be further enhanced when having the above-described bonding positions.

In the present specification, the position of substitution means positions indicated by the numbers in the following Chemical Formulae A and B.

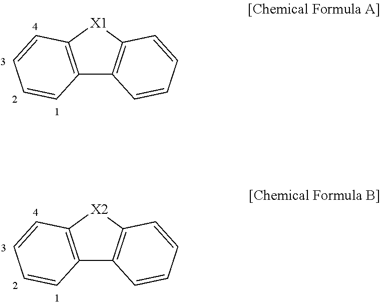

In Chemical Formulae A and B, X₁ and X₂ have the same definitions as in Chemical Formula 1, and each number means a position substituted with a substituent.

Accordingly, in the following Chemical Formula C, it may be described that the No. 4 position of

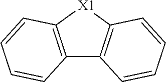

is substituted with a triazine group, and the No. 4 position of

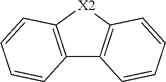

is substituted with a carbazole group.

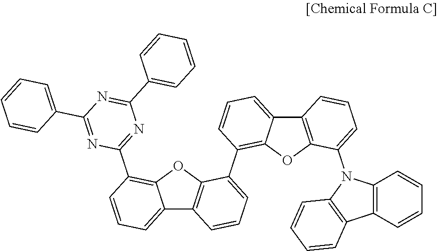 [SMILES: c1ccc(-c2nc(-c3ccccc3)nc(-c3cccc4c3oc3c(-c5cccc6c5oc5c(-n7c8ccccc8c8ccccc87)cccc56)cccc34)n2)cc1]

According to one embodiment of the present application, Chemical Formula 1 may be represented by any one of the following compounds, but is not limited thereto.

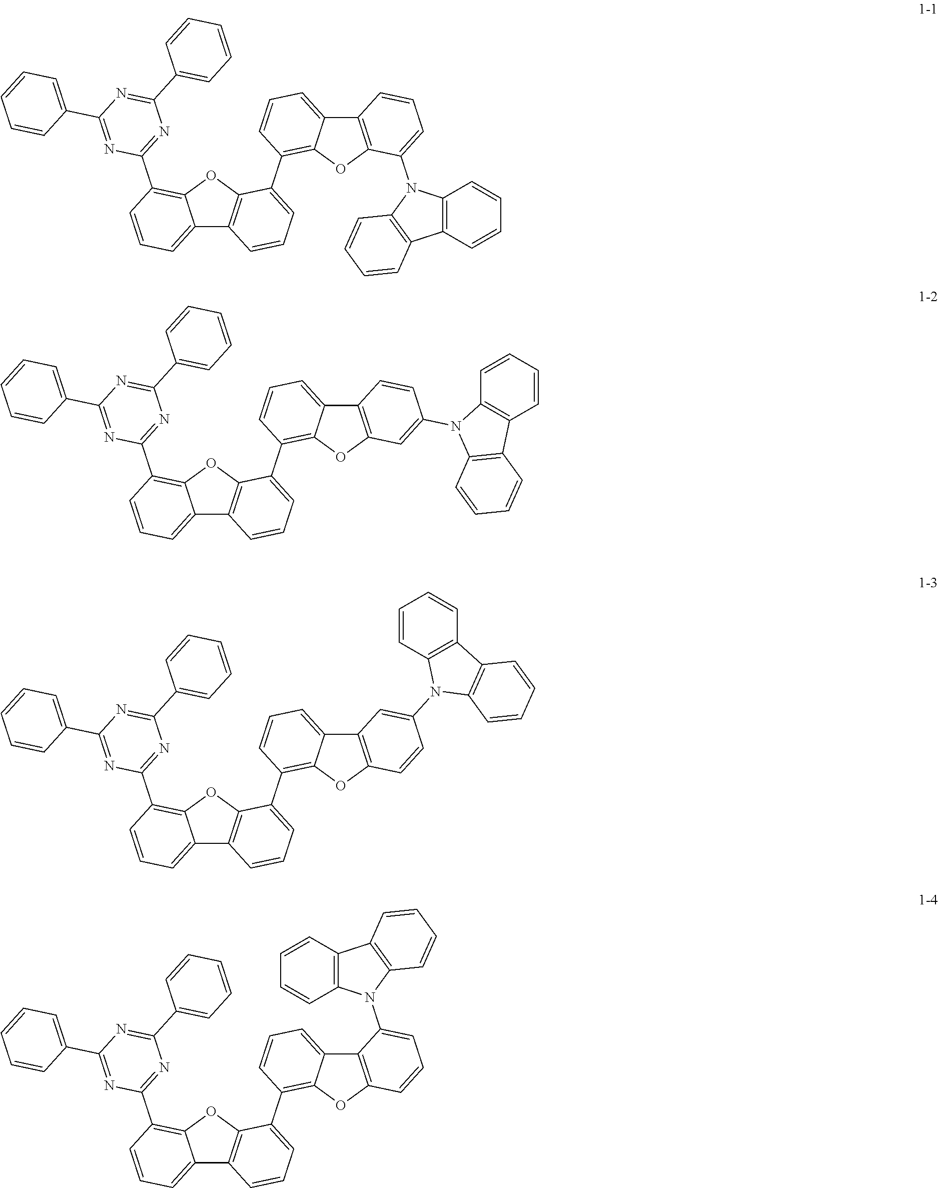 [SMILES: c1ccc(-c2nc(-c3ccccc3)nc(-c3cccc4c3oc3c(-c5cccc6c5oc5c(-n7c8ccccc8c8ccccc87)cccc56)cccc34)n2)cc1 ; c1ccc(-c2nc(-c3ccccc3)nc(-c3cccc4c3oc3c(-c5cccc6c5oc5cc(-n7c8ccccc8c8ccccc87)ccc56)cccc34)n2)cc1 ; c1ccc(-c2nc(-c3ccccc3)nc(-c3cccc4c3oc3c(-c5cccc6c5oc5ccc(-n7c8ccccc8c8ccccc87)cc56)cccc34)n2)cc1 ; c1ccc(-c2nc(-c3ccccc3)nc(-c3cccc4c3oc3c(-c5cccc6c5oc5cccc(-n7c8ccccc8c8ccccc87)c56)cccc34)n2)cc1]

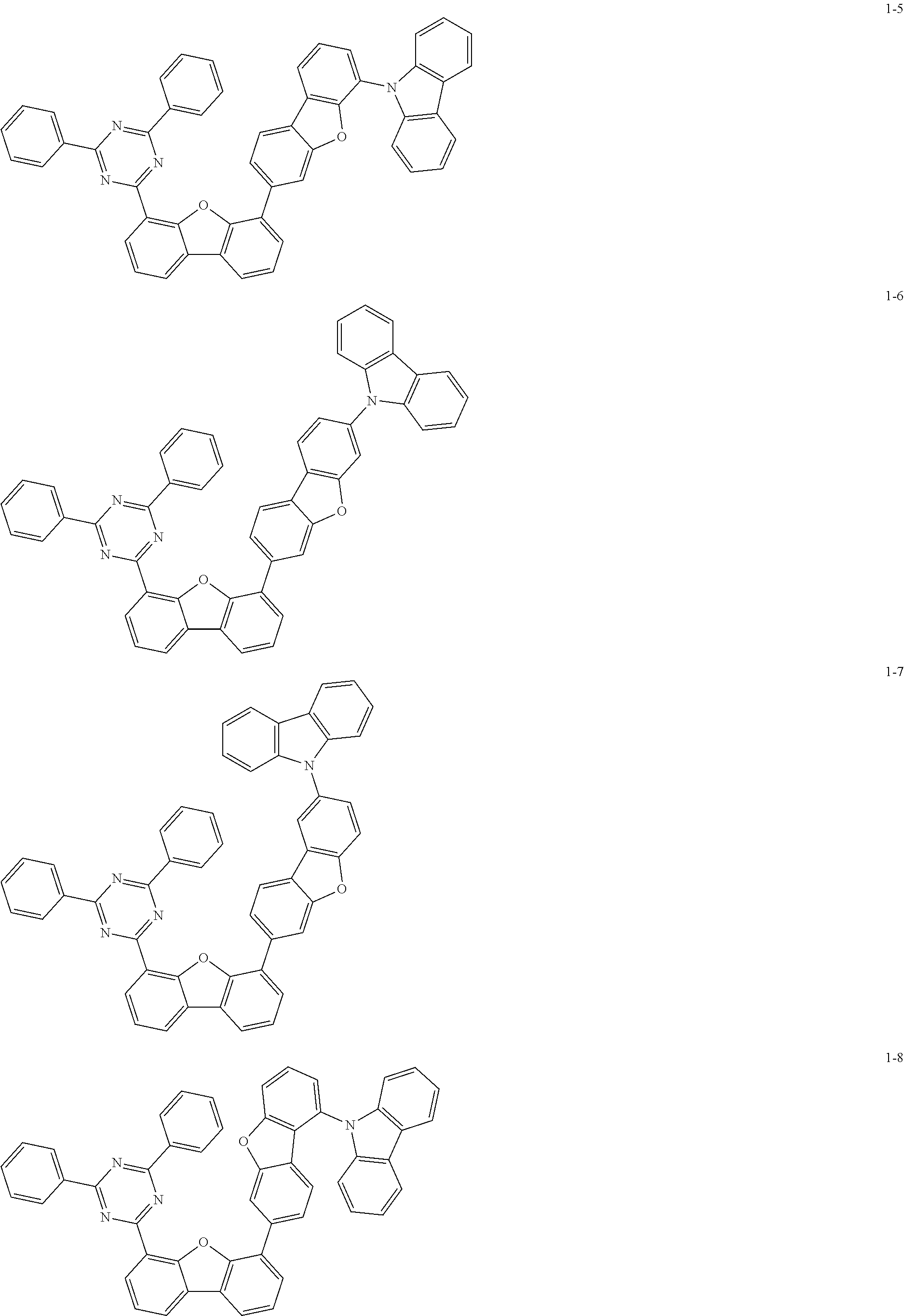 [SMILES: c1ccc(-c2nc(-c3ccccc3)nc(-c3cccc4c3oc3c(-c5ccc6c(c5)oc5c(-n7c8ccccc8c8ccccc87)cccc56)cccc34)n2)cc1 ; c1ccc(-c2nc(-c3ccccc3)nc(-c3cccc4c3oc3c(-c5ccc6c(c5)oc5cc(-n7c8ccccc8c8ccccc87)ccc56)cccc34)n2)cc1 ; c1ccc(-c2nc(-c3ccccc3)nc(-c3cccc4c3oc3c(-c5ccc6c(c5)oc5ccc(-n7c8ccccc8c8ccccc87)cc56)cccc34)n2)cc1 ; c1ccc(-c2nc(-c3ccccc3)nc(-c3cccc4c3oc3c(-c5ccc6c(c5)oc5cccc(-n7c8ccccc8c8ccccc87)c56)cccc34)n2)cc1]

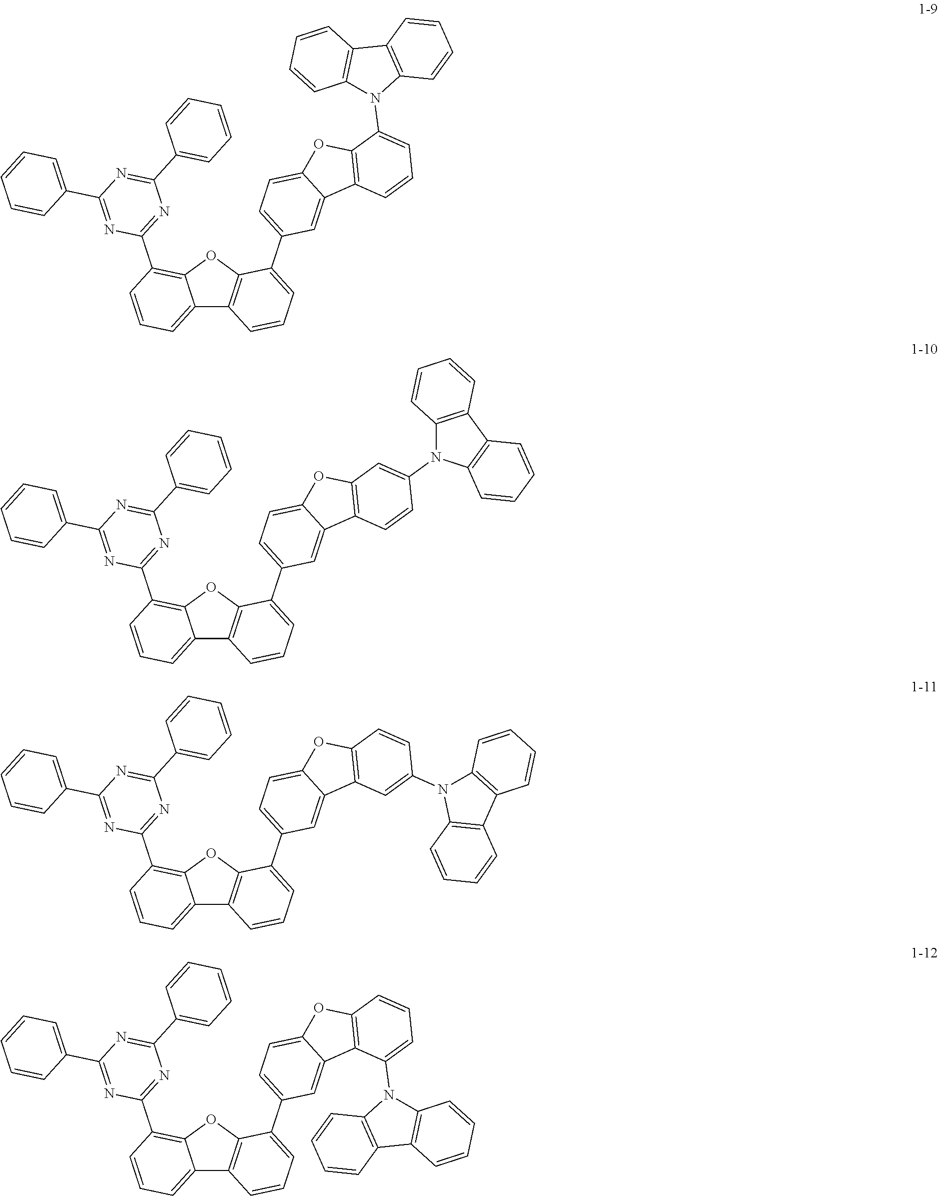

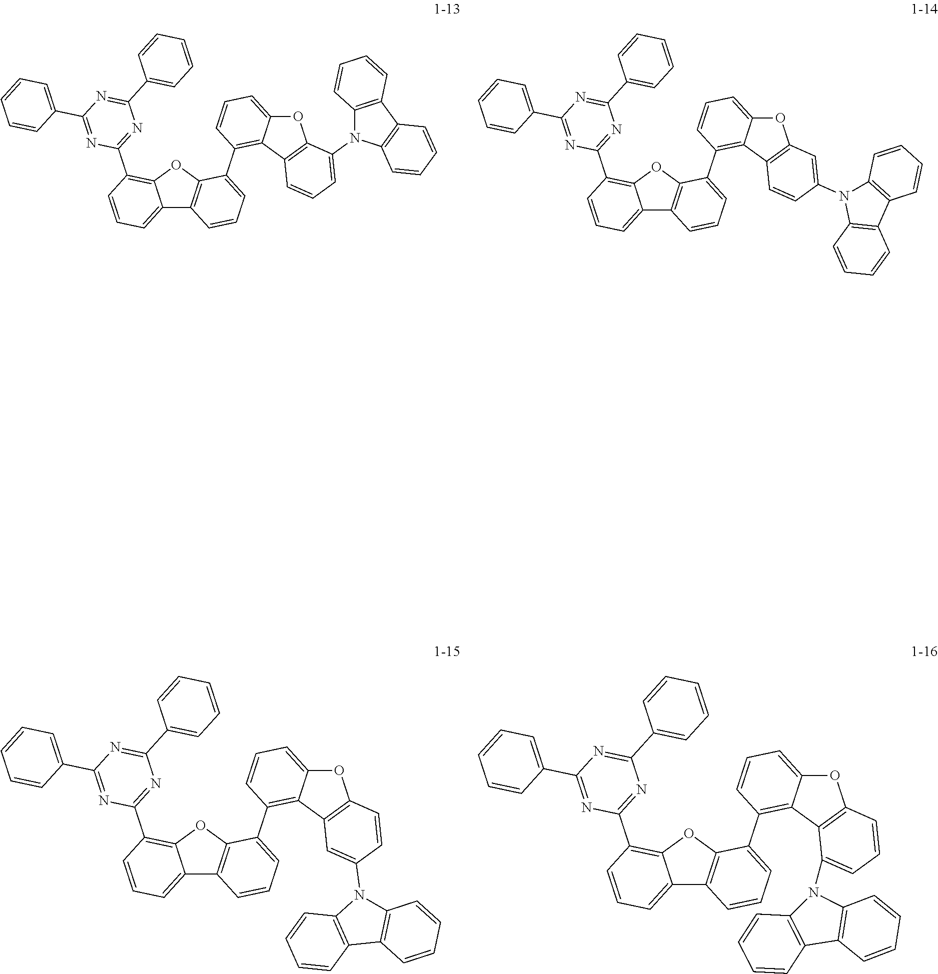 [SMILES: c1ccc(-c2nc(-c3ccccc3)nc(-c3cccc4c3oc3c(-c5cccc6oc7c(-n8c9ccccc9c9ccccc98)cccc7c56)cccc34)n2)cc1 ; c1ccc(-c2nc(-c3ccccc3)nc(-c3cccc4c3oc3c(-c5cccc6oc7cc(-n8c9ccccc9c9ccccc98)ccc7c56)cccc34)n2)cc1 ; c1ccc(-c2nc(-c3ccccc3)nc(-c3cccc4c3oc3c(-c5cccc6oc7ccc(-n8c9ccccc9c9ccccc98)cc7c56)cccc34)n2)cc1 ; c1ccc(-c2nc(-c3ccccc3)nc(-c3cccc4c3oc3c(-c5cccc6oc7cccc(-n8c9ccccc9c9ccccc98)c7c56)cccc34)n2)cc1]

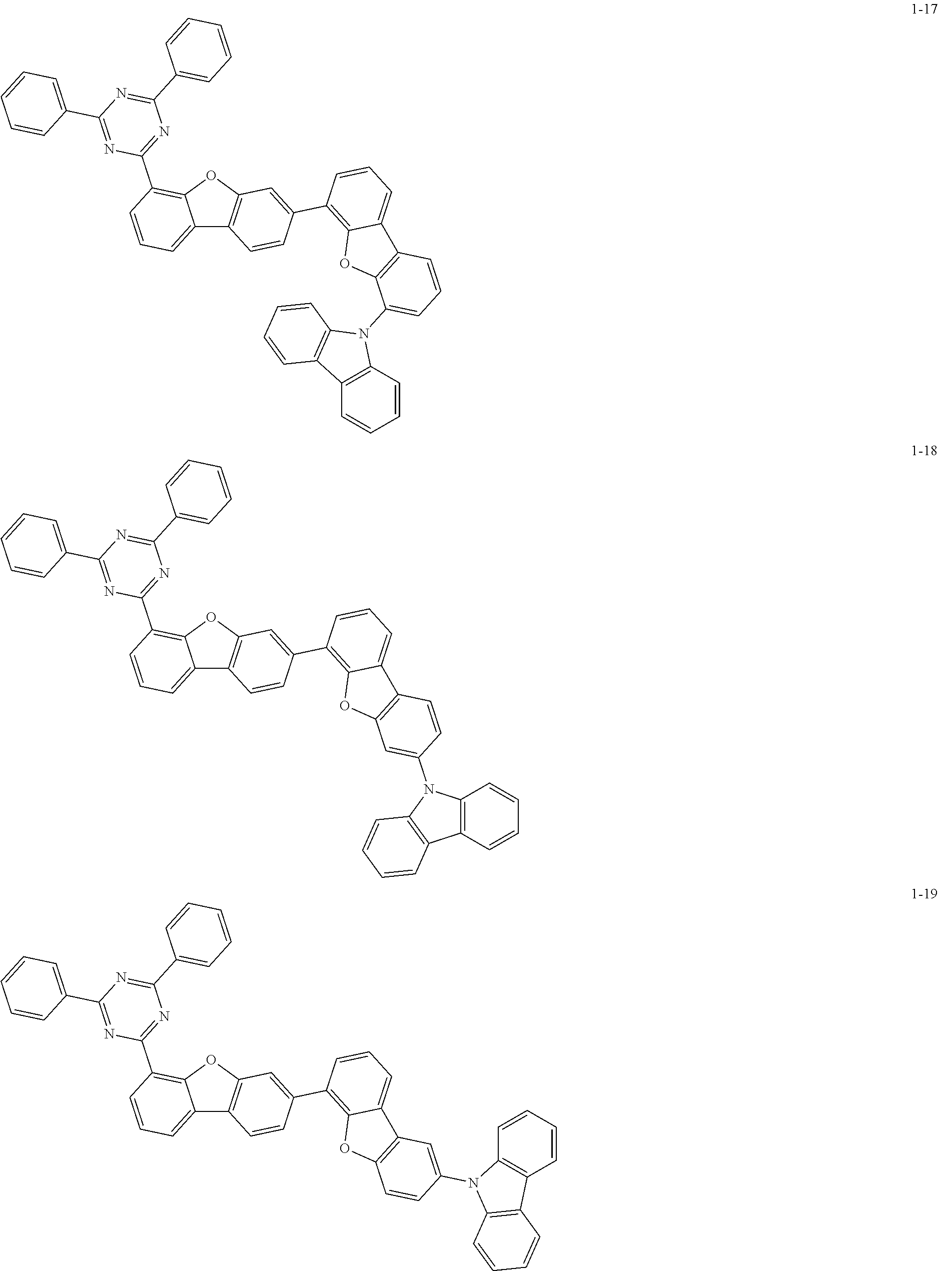 [SMILES: c1ccc(-c2nc(-c3ccccc3)nc(-c3cccc4c3oc3cc(-c5cccc6c5oc5c(-n7c8ccccc8c8ccccc87)cccc56)ccc34)n2)cc1 ; c1ccc(-c2nc(-c3ccccc3)nc(-c3cccc4c3oc3cc(-c5cccc6c5oc5cc(-n7c8ccccc8c8ccccc87)ccc56)ccc34)n2)cc1 ; c1ccc(-c2nc(-c3ccccc3)nc(-c3cccc4c3oc3cc(-c5cccc6c5oc5ccc(-n7c8ccccc8c8ccccc87)cc56)ccc34)n2)cc1]

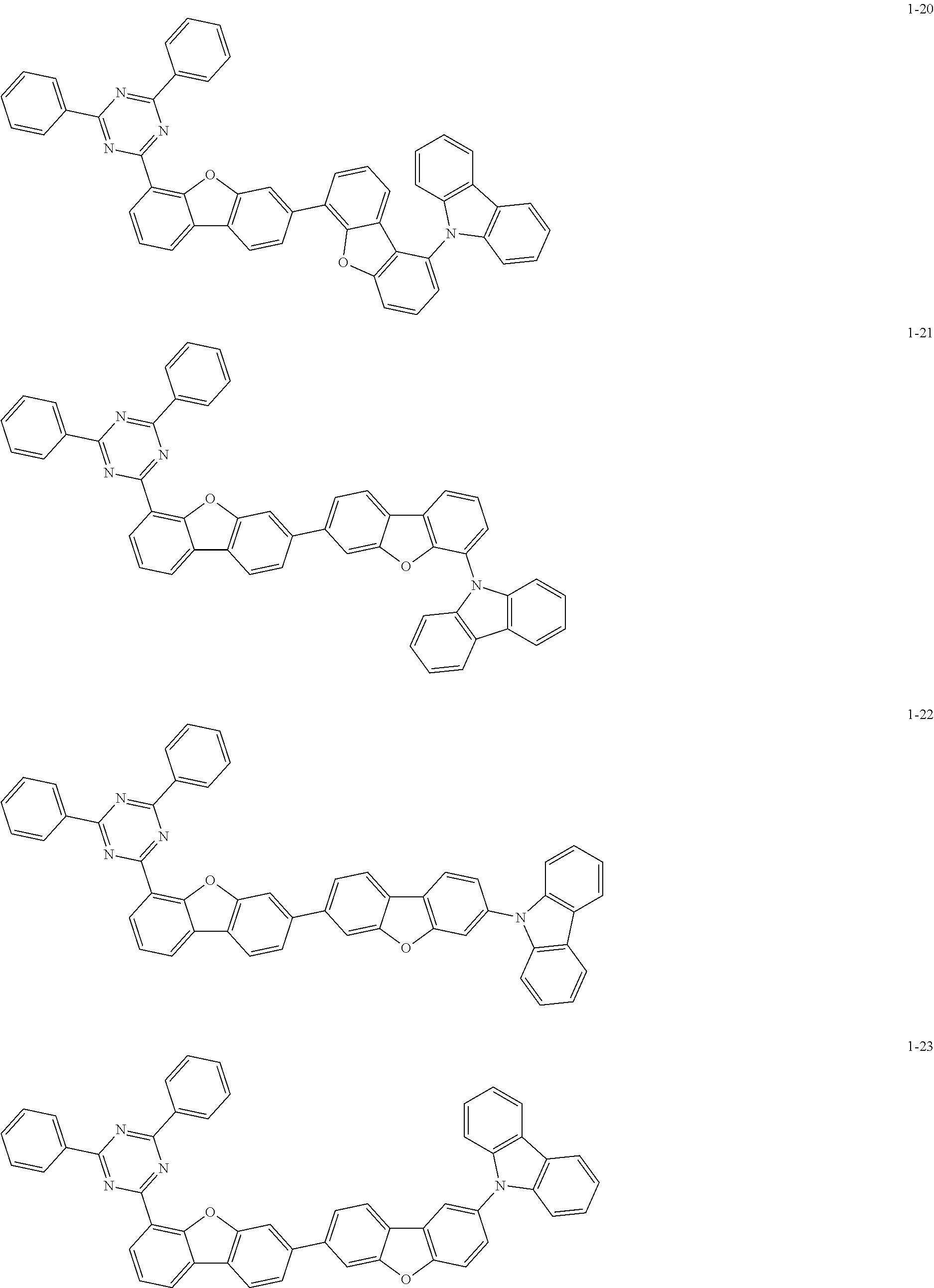 [SMILES: c1ccc(-c2nc(-c3ccccc3)nc(-c3cccc4c3oc3cc(-c5cccc6c5oc5cccc(-n7c8ccccc8c8ccccc87)c56)ccc34)n2)cc1 ; c1ccc(-c2nc(-c3ccccc3)nc(-c3cccc4c3oc3cc(-c5ccc6c(c5)oc5c(-n7c8ccccc8c8ccccc87)cccc56)ccc34)n2)cc1 ; c1ccc(-c2nc(-c3ccccc3)nc(-c3cccc4c3oc3cc(-c5ccc6c(c5)oc5cc(-n7c8ccccc8c8ccccc87)ccc56)ccc34)n2)cc1 ; c1ccc(-c2nc(-c3ccccc3)nc(-c3cccc4c3oc3cc(-c5ccc6c(c5)oc5ccc(-n7c8ccccc8c8ccccc87)cc56)ccc34)n2)cc1]

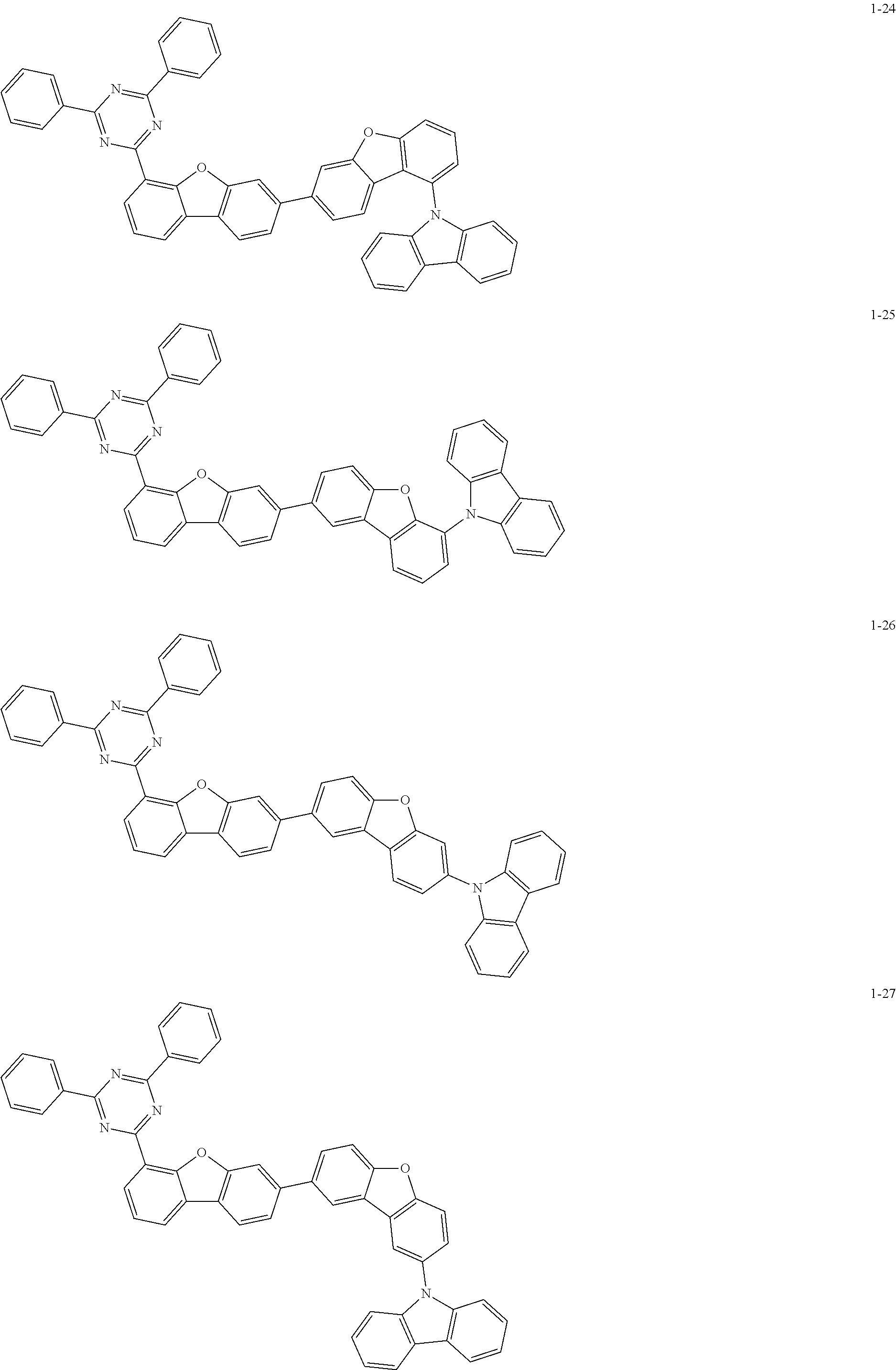 [SMILES: c1ccc(-c2nc(-c3ccccc3)nc(-c3cccc4c3oc3cc(-c5ccc6c(c5)oc5cccc(-n7c8ccccc8c8ccccc87)c56)ccc34)n2)cc1 ; c1ccc(-c2nc(-c3ccccc3)nc(-c3cccc4c3oc3cc(-c5ccc6oc7c(-n8c9ccccc9c9ccccc98)cccc7c6c5)ccc34)n2)cc1 ; c1ccc(-c2nc(-c3ccccc3)nc(-c3cccc4c3oc3cc(-c5ccc6oc7cc(-n8c9ccccc9c9ccccc98)ccc7c6c5)ccc34)n2)cc1 ; c1ccc(-c2nc(-c3ccccc3)nc(-c3cccc4c3oc3cc(-c5ccc6oc7ccc(-n8c9ccccc9c9ccccc98)cc7c6c5)ccc34)n2)cc1]

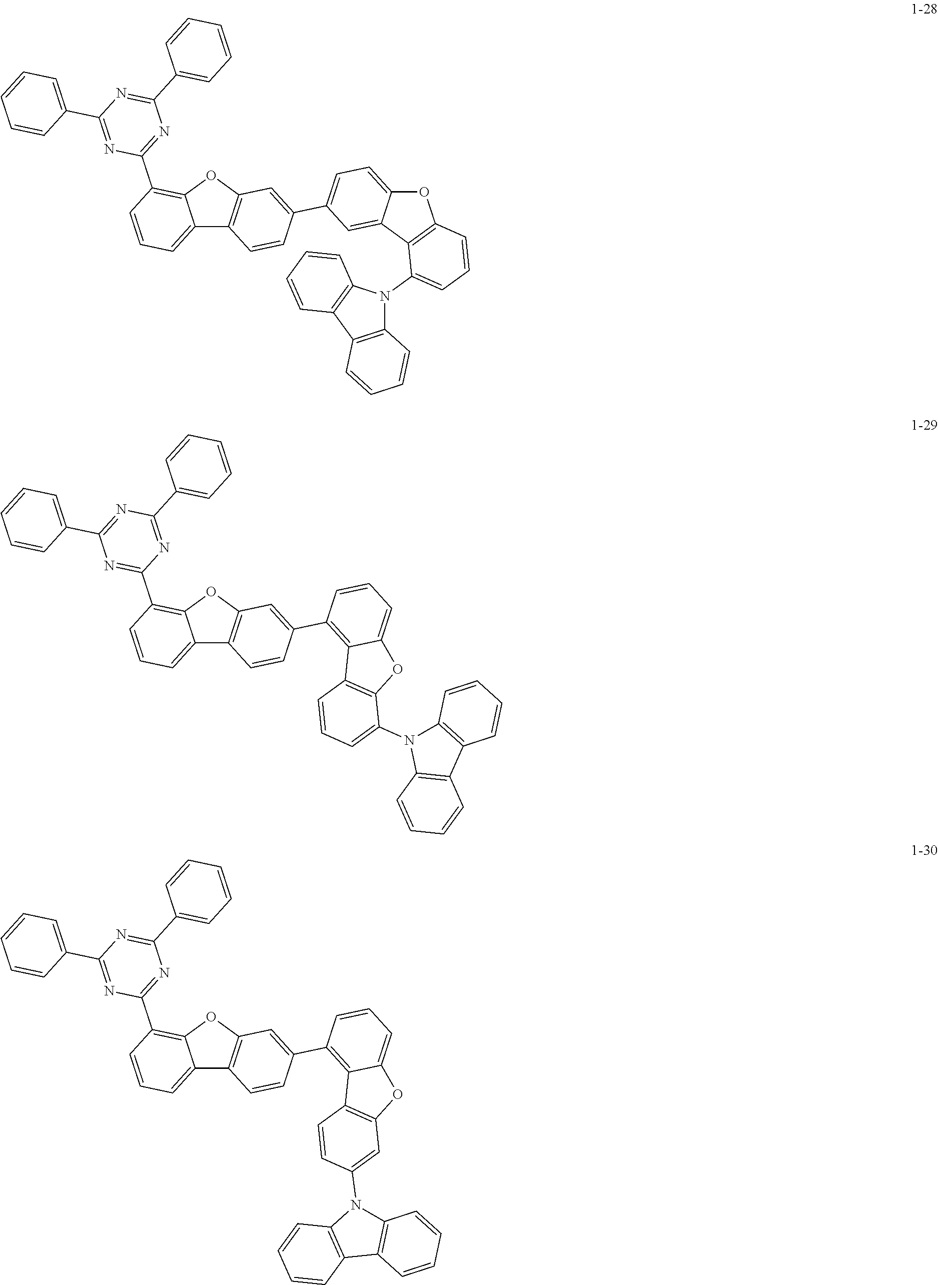 [SMILES: c1ccc(-c2nc(-c3ccccc3)nc(-c3cccc4c3oc3cc(-c5ccc6oc7cccc(-n8c9ccccc9c9ccccc98)c7c6c5)ccc34)n2)cc1 ; c1ccc(-c2nc(-c3ccccc3)nc(-c3cccc4c3oc3cc(-c5cccc6oc7c(-n8c9ccccc9c9ccccc98)cccc7c56)ccc34)n2)cc1 ; c1ccc(-c2nc(-c3ccccc3)nc(-c3cccc4c3oc3cc(-c5cccc6oc7cc(-n8c9ccccc9c9ccccc98)ccc7c56)ccc34)n2)cc1]

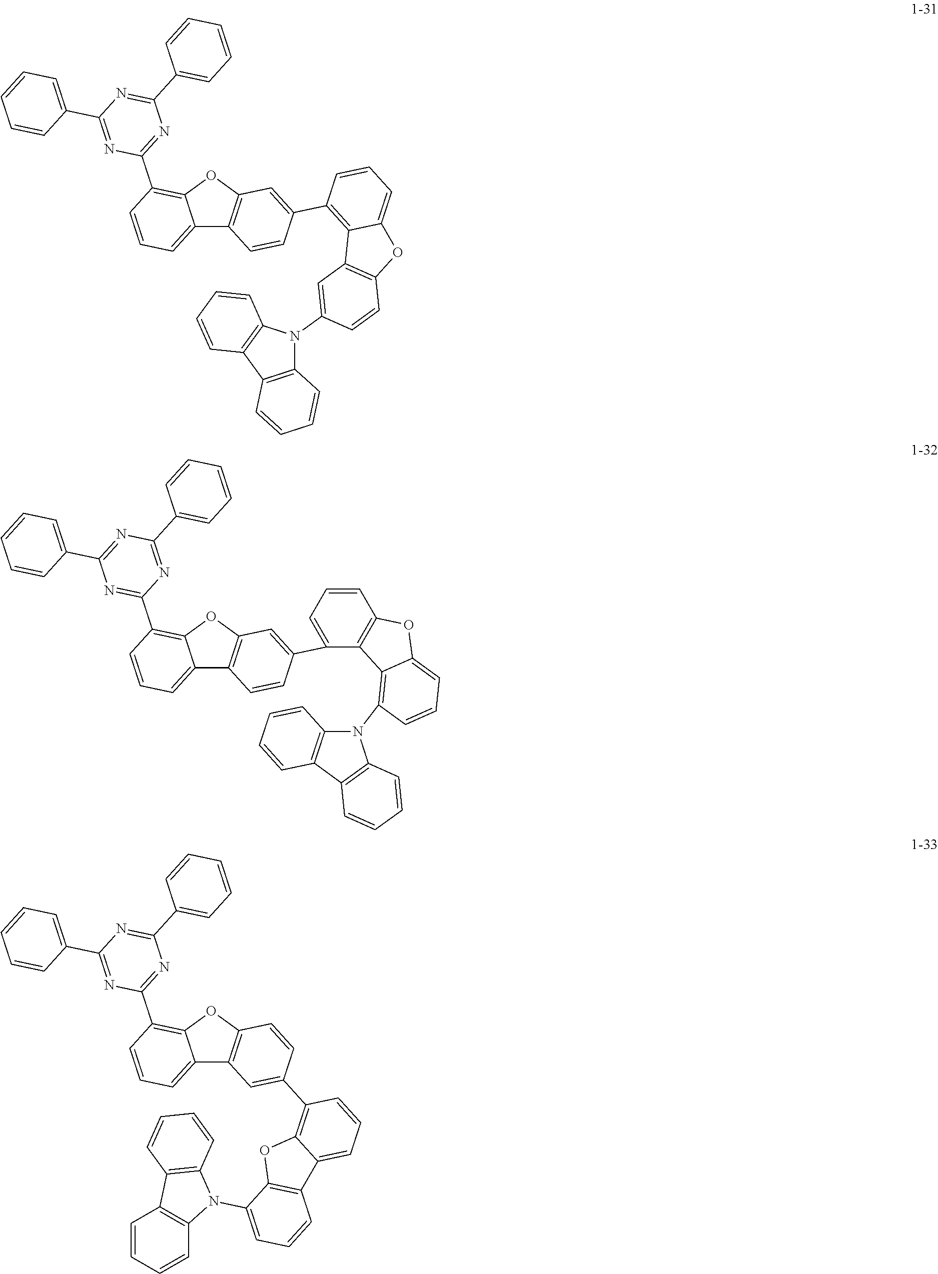 [SMILES: c1ccc(-c2nc(-c3ccccc3)nc(-c3cccc4c3oc3cc(-c5cccc6oc7ccc(-n8c9ccccc9c9ccccc98)cc7c56)ccc34)n2)cc1 ; c1ccc(-c2nc(-c3ccccc3)nc(-c3cccc4c3oc3cc(-c5cccc6oc7cccc(-n8c9ccccc9c9ccccc98)c7c56)ccc34)n2)cc1 ; c1ccc(-c2nc(-c3ccccc3)nc(-c3cccc4c3oc3ccc(-c5cccc6c5oc5c(-n7c8ccccc8c8ccccc87)cccc56)cc34)n2)cc1]

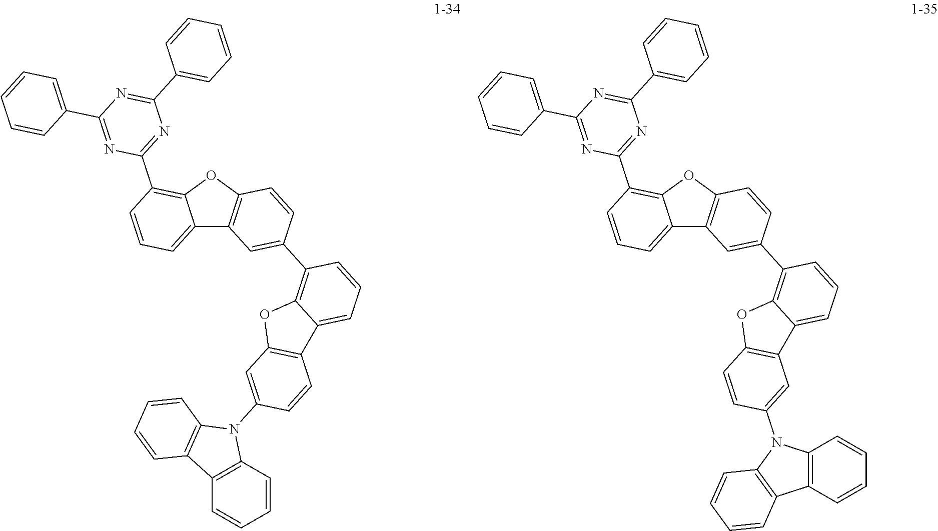 [SMILES: c1ccc(-c2nc(-c3ccccc3)nc(-c3cccc4c3oc3ccc(-c5cccc6c5oc5cc(-n7c8ccccc8c8ccccc87)ccc56)cc34)n2)cc1 ; c1ccc(-c2nc(-c3ccccc3)nc(-c3cccc4c3oc3ccc(-c5cccc6c5oc5ccc(-n7c8ccccc8c8ccccc87)cc56)cc34)n2)cc1]

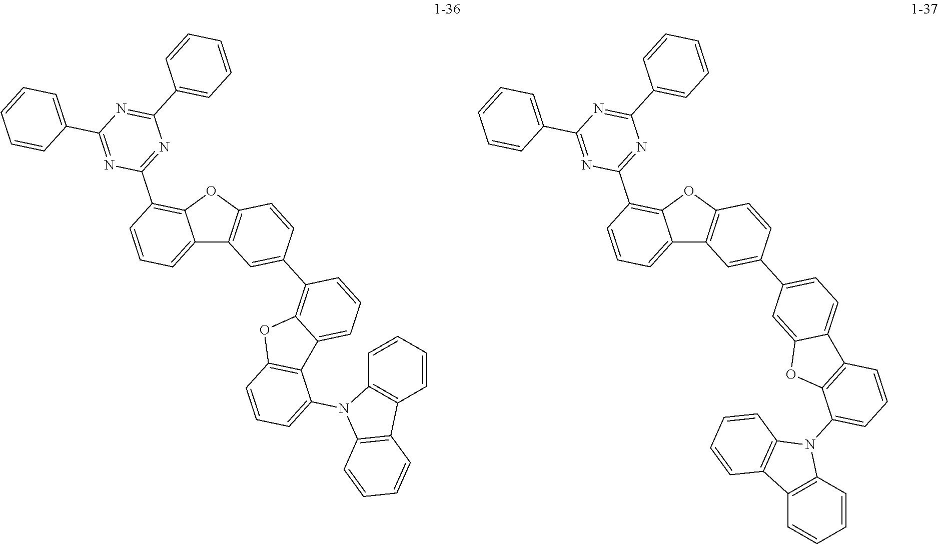 [SMILES: c1ccc(-c2nc(-c3ccccc3)nc(-c3cccc4c3oc3ccc(-c5cccc6c5oc5cccc(-n7c8ccccc8c8ccccc87)c56)cc34)n2)cc1 ; c1ccc(-c2nc(-c3ccccc3)nc(-c3cccc4c3oc3ccc(-c5ccc6c(c5)oc5c(-n7c8ccccc8c8ccccc87)cccc56)cc34)n2)cc1]

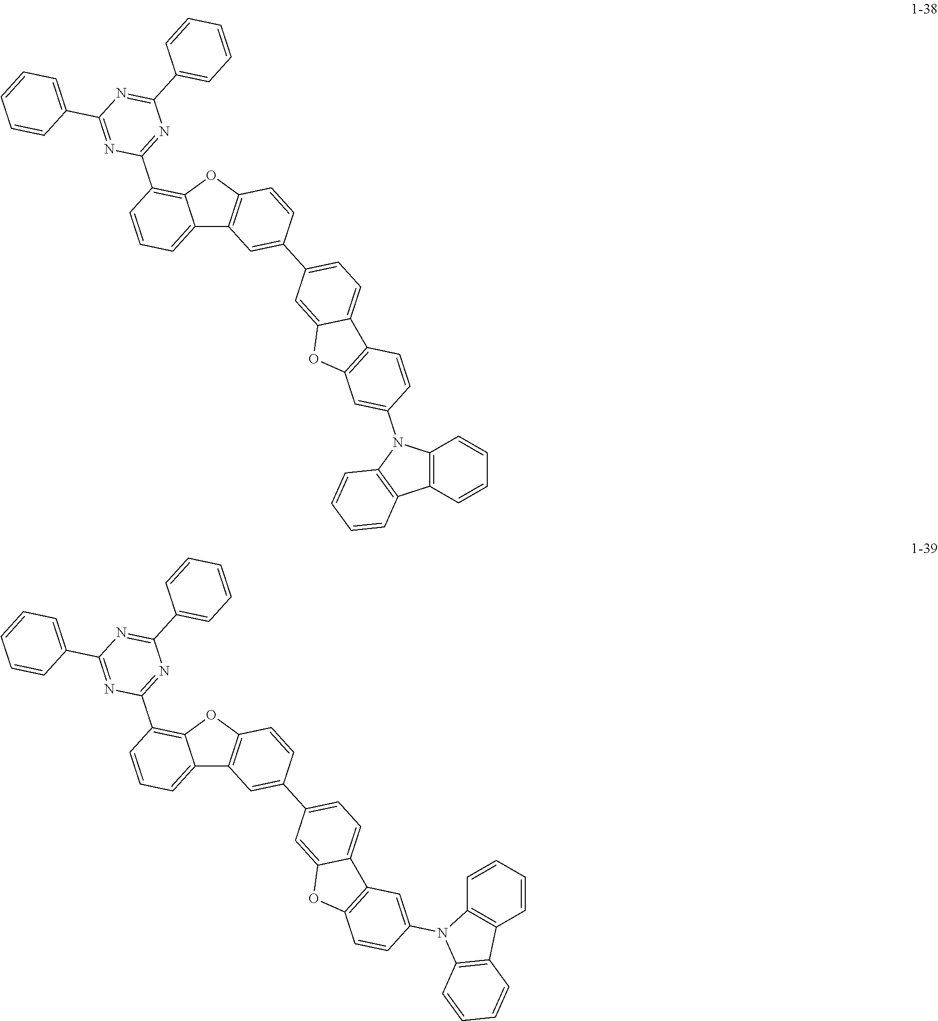 [SMILES: c1ccc(-c2nc(-c3ccccc3)nc(-c3cccc4c3oc3ccc(-c5ccc6c(c5)oc5cc(-n7c8ccccc8c8ccccc87)ccc56)cc34)n2)cc1 ; c1ccc(-c2nc(-c3ccccc3)nc(-c3cccc4c3oc3ccc(-c5ccc6c(c5)oc5ccc(-n7c8ccccc8c8ccccc87)cc56)cc34)n2)cc1]

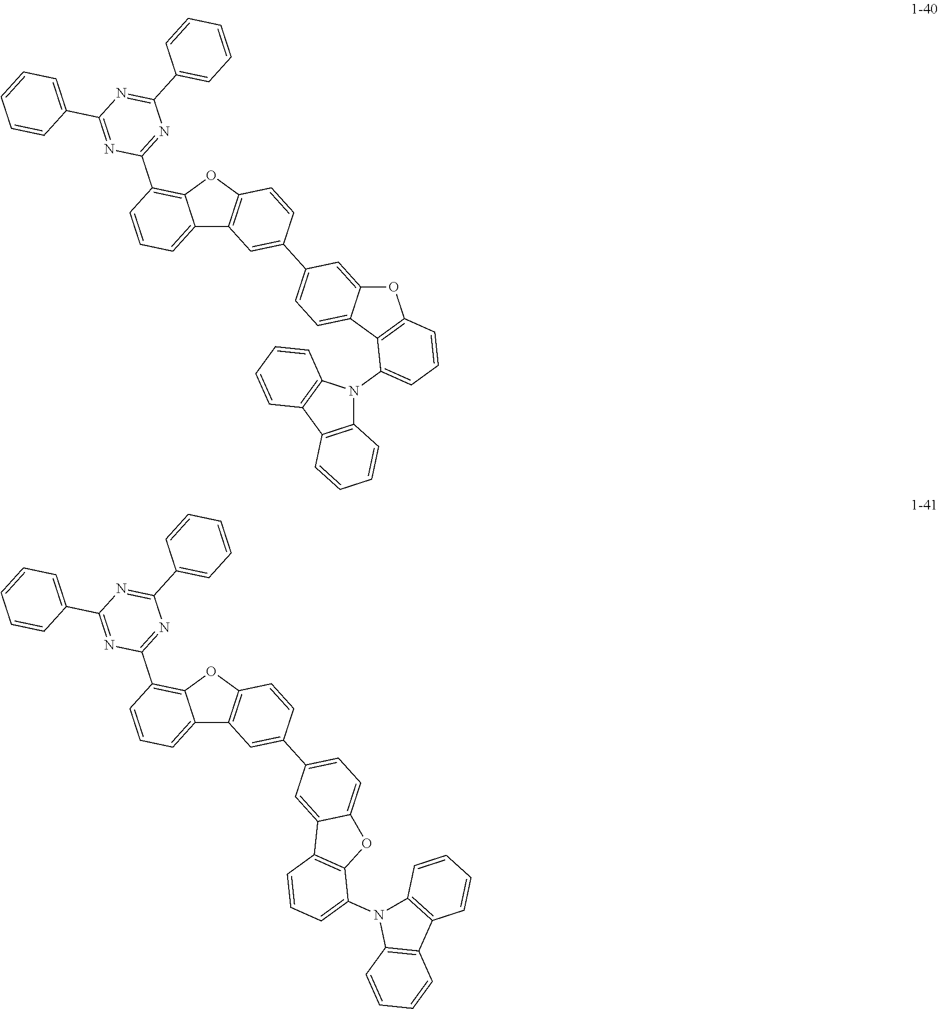 [SMILES: c1ccc(-c2nc(-c3ccccc3)nc(-c3cccc4c3oc3ccc(-c5ccc6c(c5)oc5cccc(-n7c8ccccc8c8ccccc87)c56)cc34)n2)cc1 ; c1ccc(-c2nc(-c3ccccc3)nc(-c3cccc4c3oc3ccc(-c5ccc6oc7c(-n8c9ccccc9c9ccccc98)cccc7c6c5)cc34)n2)cc1]

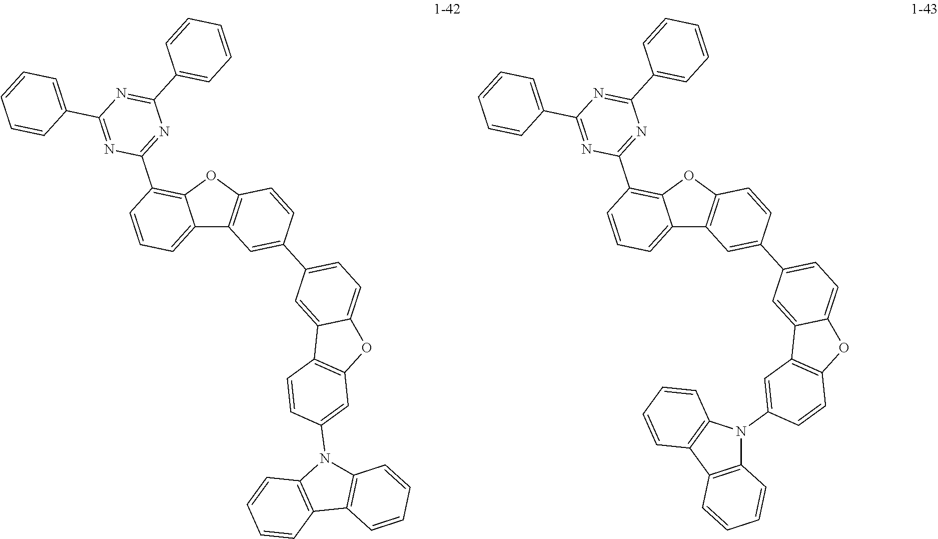 [SMILES: c1ccc(-c2nc(-c3ccccc3)nc(-c3cccc4c3oc3ccc(-c5ccc6oc7cc(-n8c9ccccc9c9ccccc98)ccc7c6c5)cc34)n2)cc1 ; c1ccc(-c2nc(-c3ccccc3)nc(-c3cccc4c3oc3ccc(-c5ccc6oc7ccc(-n8c9ccccc9c9ccccc98)cc7c6c5)cc34)n2)cc1]

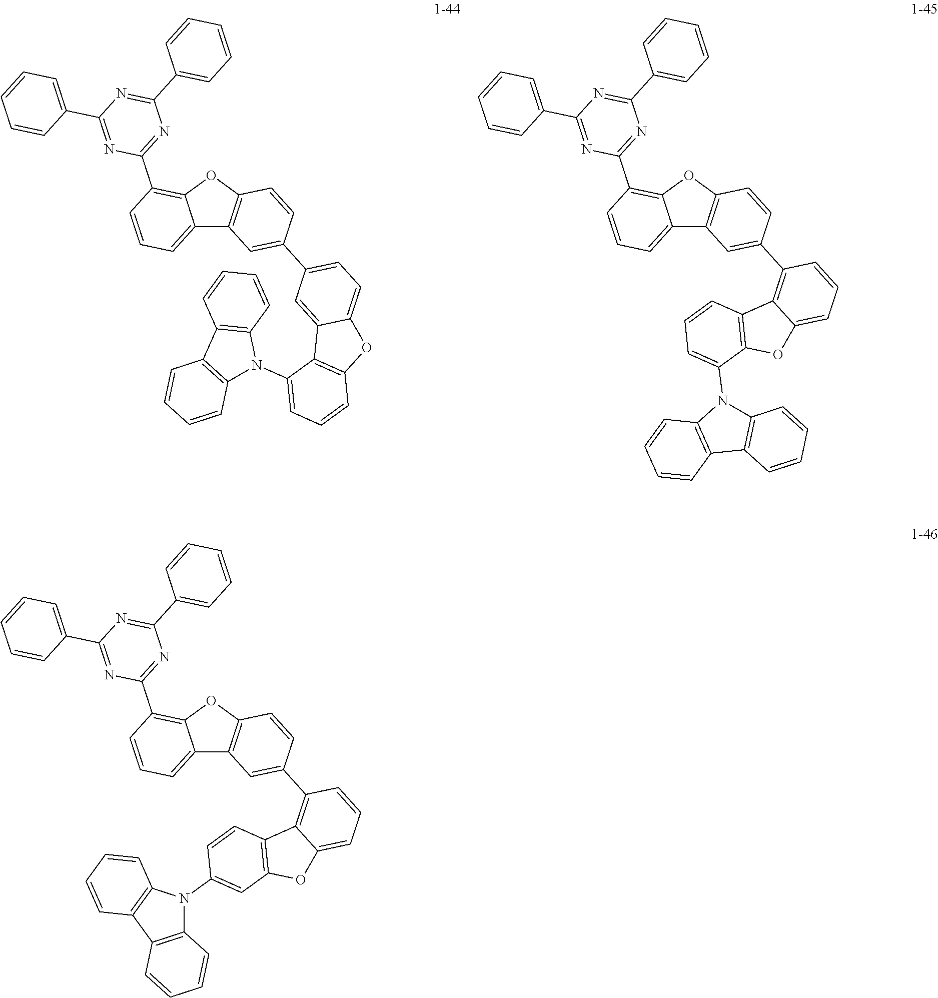 [SMILES: c1ccc(-c2nc(-c3ccccc3)nc(-c3cccc4c3oc3ccc(-c5ccc6oc7cccc(-n8c9ccccc9c9ccccc98)c7c6c5)cc34)n2)cc1 ; c1ccc(-c2nc(-c3ccccc3)nc(-c3cccc4c3oc3ccc(-c5cccc6oc7c(-n8c9ccccc9c9ccccc98)cccc7c56)cc34)n2)cc1 ; c1ccc(-c2nc(-c3ccccc3)nc(-c3cccc4c3oc3ccc(-c5cccc6oc7cc(-n8c9ccccc9c9ccccc98)ccc7c56)cc34)n2)cc1]

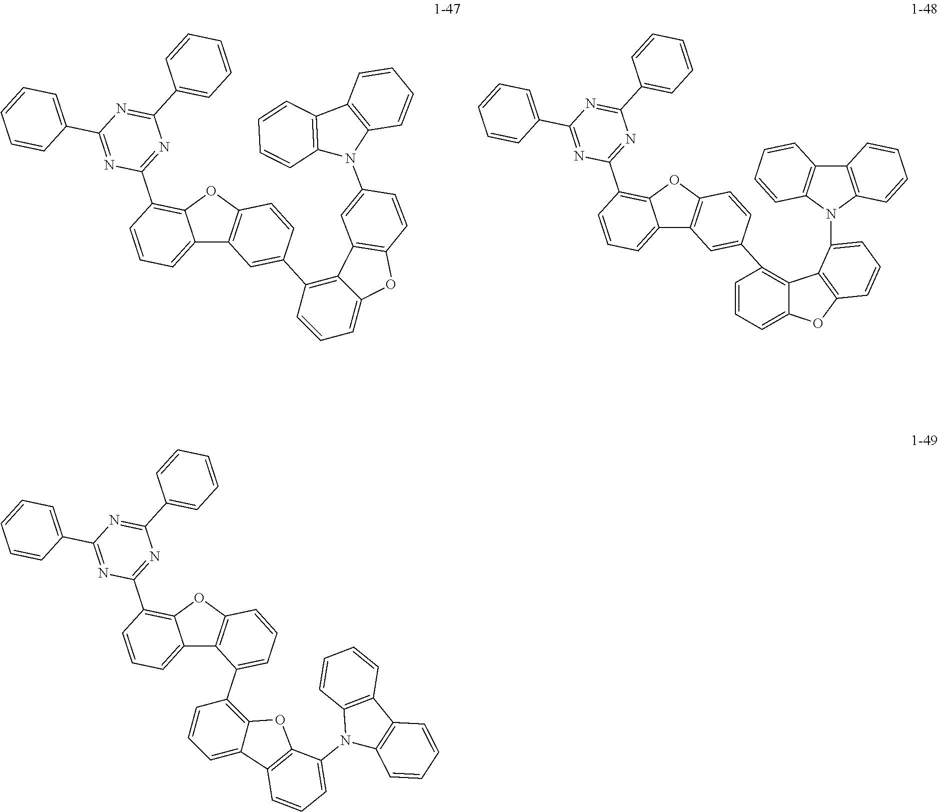 [SMILES: c1ccc(-c2nc(-c3ccccc3)nc(-c3cccc4c3oc3ccc(-c5cccc6oc7ccc(-n8c9ccccc9c9ccccc98)cc7c56)cc34)n2)cc1 ; c1ccc(-c2nc(-c3ccccc3)nc(-c3cccc4c3oc3ccc(-c5cccc6oc7cccc(-n8c9ccccc9c9ccccc98)c7c56)cc34)n2)cc1 ; c1ccc(-c2nc(-c3ccccc3)nc(-c3cccc4c3oc3cccc(-c5cccc6c5oc5c(-n7c8ccccc8c8ccccc87)cccc56)c34)n2)cc1]

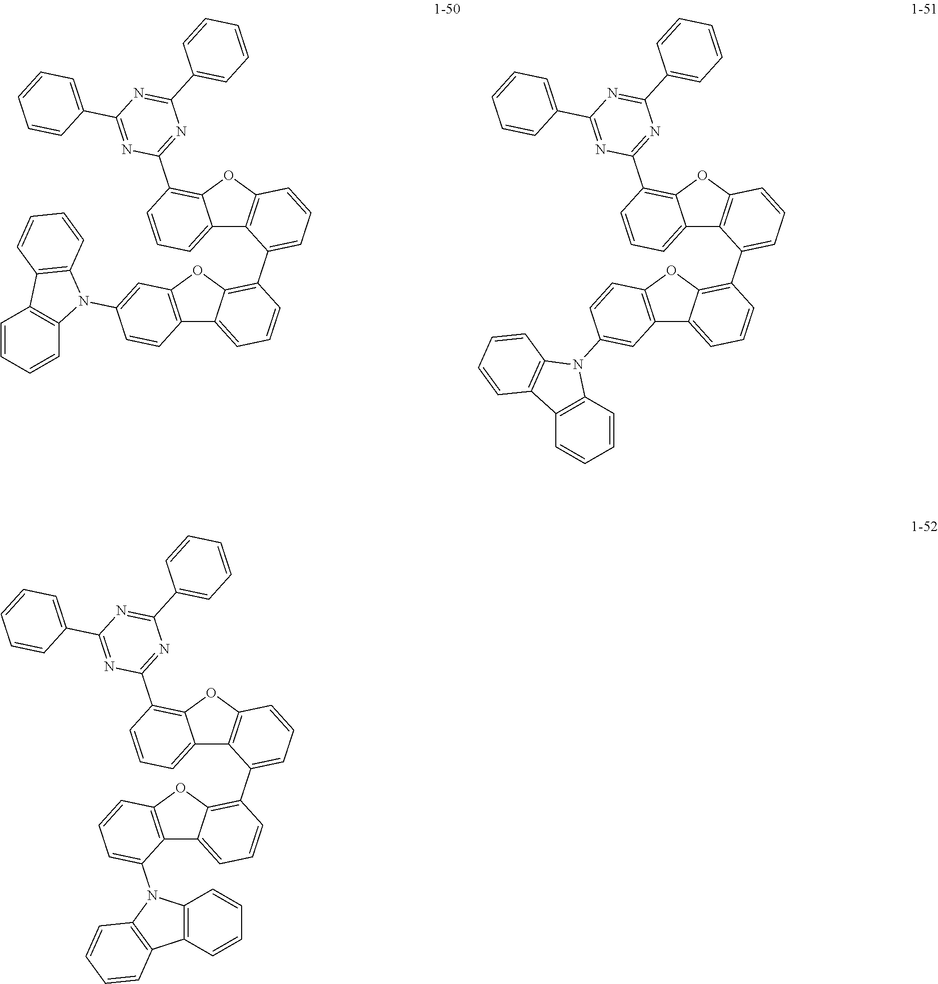 [SMILES: c1ccc(-c2nc(-c3ccccc3)nc(-c3cccc4c3oc3cccc(-c5cccc6c5oc5cc(-n7c8ccccc8c8ccccc87)ccc56)c34)n2)cc1 ; c1ccc(-c2nc(-c3ccccc3)nc(-c3cccc4c3oc3cccc(-c5cccc6c5oc5ccc(-n7c8ccccc8c8ccccc87)cc56)c34)n2)cc1 ; c1ccc(-c2nc(-c3ccccc3)nc(-c3cccc4c3oc3cccc(-c5cccc6c5oc5cccc(-n7c8ccccc8c8ccccc87)c56)c34)n2)cc1]

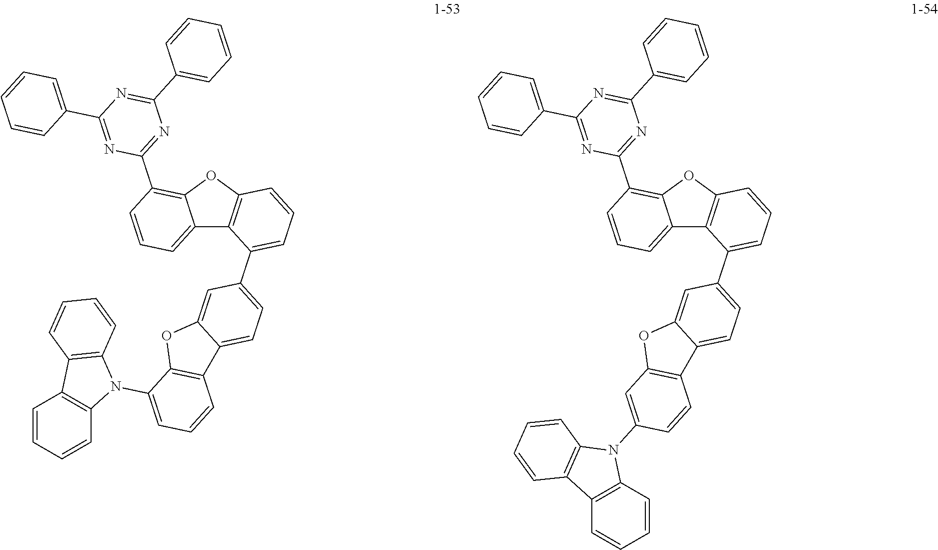 [SMILES: c1ccc(-c2nc(-c3ccccc3)nc(-c3cccc4c3oc3cccc(-c5ccc6c(c5)oc5c(-n7c8ccccc8c8ccccc87)cccc56)c34)n2)cc1 ; c1ccc(-c2nc(-c3ccccc3)nc(-c3cccc4c3oc3cccc(-c5ccc6c(c5)oc5cc(-n7c8ccccc8c8ccccc87)ccc56)c34)n2)cc1]

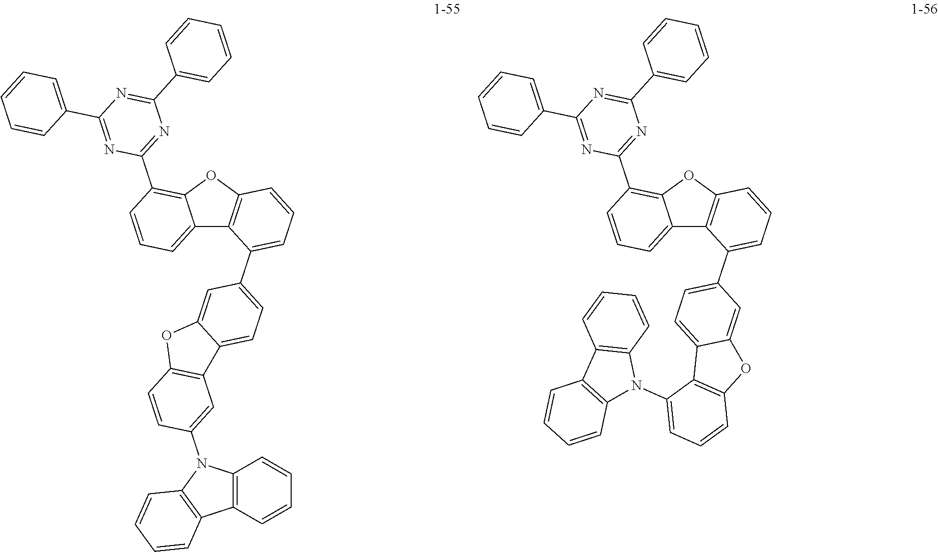 [SMILES: c1ccc(-c2nc(-c3ccccc3)nc(-c3cccc4c3oc3cccc(-c5ccc6c(c5)oc5ccc(-n7c8ccccc8c8ccccc87)cc56)c34)n2)cc1 ; c1ccc(-c2nc(-c3ccccc3)nc(-c3cccc4c3oc3cccc(-c5ccc6c(c5)oc5cccc(-n7c8ccccc8c8ccccc87)c56)c34)n2)cc1]

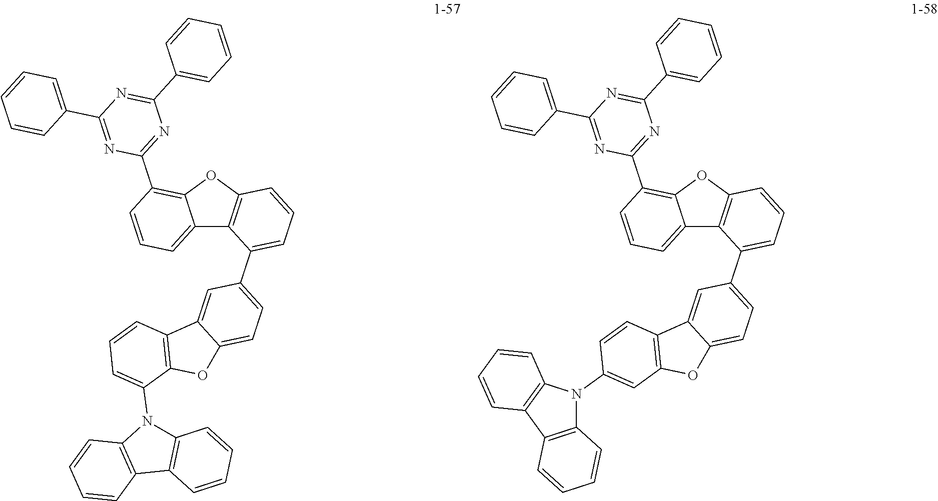 [SMILES: c1ccc(-c2nc(-c3ccccc3)nc(-c3cccc4c3oc3cccc(-c5ccc6oc7c(-n8c9ccccc9c9ccccc98)cccc7c6c5)c34)n2)cc1 ; c1ccc(-c2nc(-c3ccccc3)nc(-c3cccc4c3oc3cccc(-c5ccc6oc7cc(-n8c9ccccc9c9ccccc98)ccc7c6c5)c34)n2)cc1]

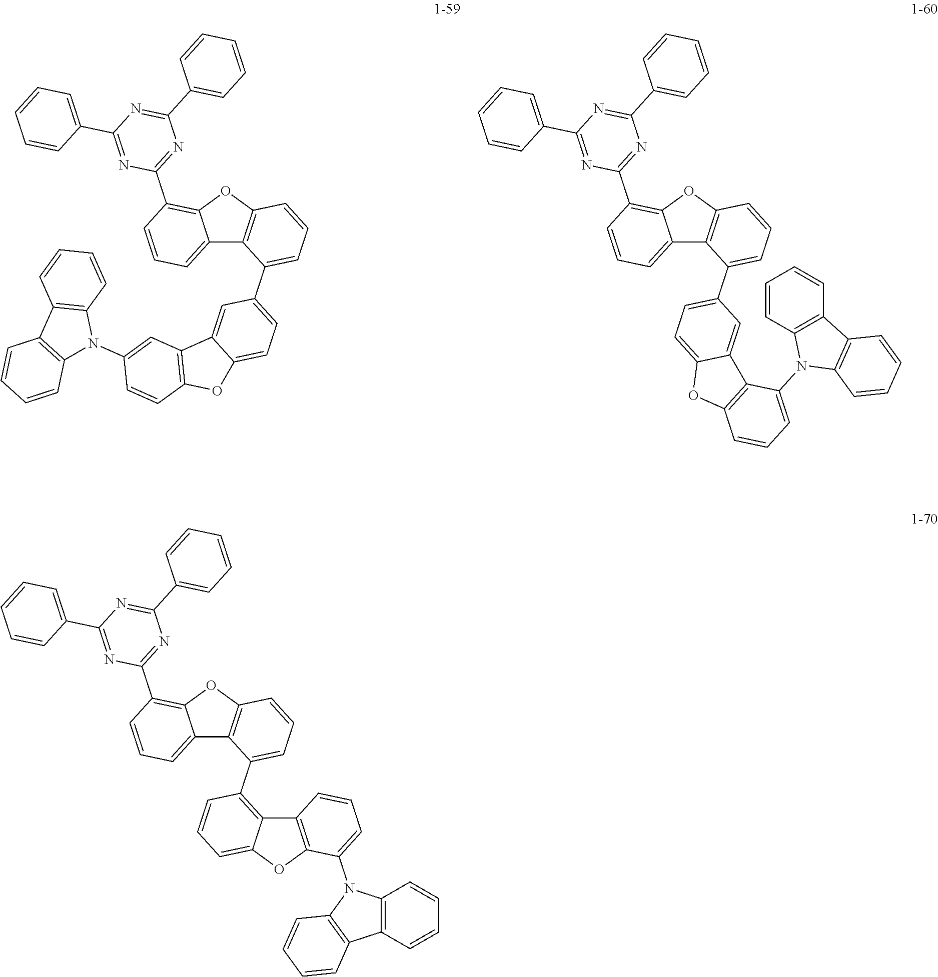 [SMILES: c1ccc(-c2nc(-c3ccccc3)nc(-c3cccc4c3oc3cccc(-c5ccc6oc7ccc(-n8c9ccccc9c9ccccc98)cc7c6c5)c34)n2)cc1 ; c1ccc(-c2nc(-c3ccccc3)nc(-c3cccc4c3oc3cccc(-c5ccc6oc7cccc(-n8c9ccccc9c9ccccc98)c7c6c5)c34)n2)cc1 ; c1ccc(-c2nc(-c3ccccc3)nc(-c3cccc4c3oc3cccc(-c5cccc6oc7c(-n8c9ccccc9c9ccccc98)cccc7c56)c34)n2)cc1]

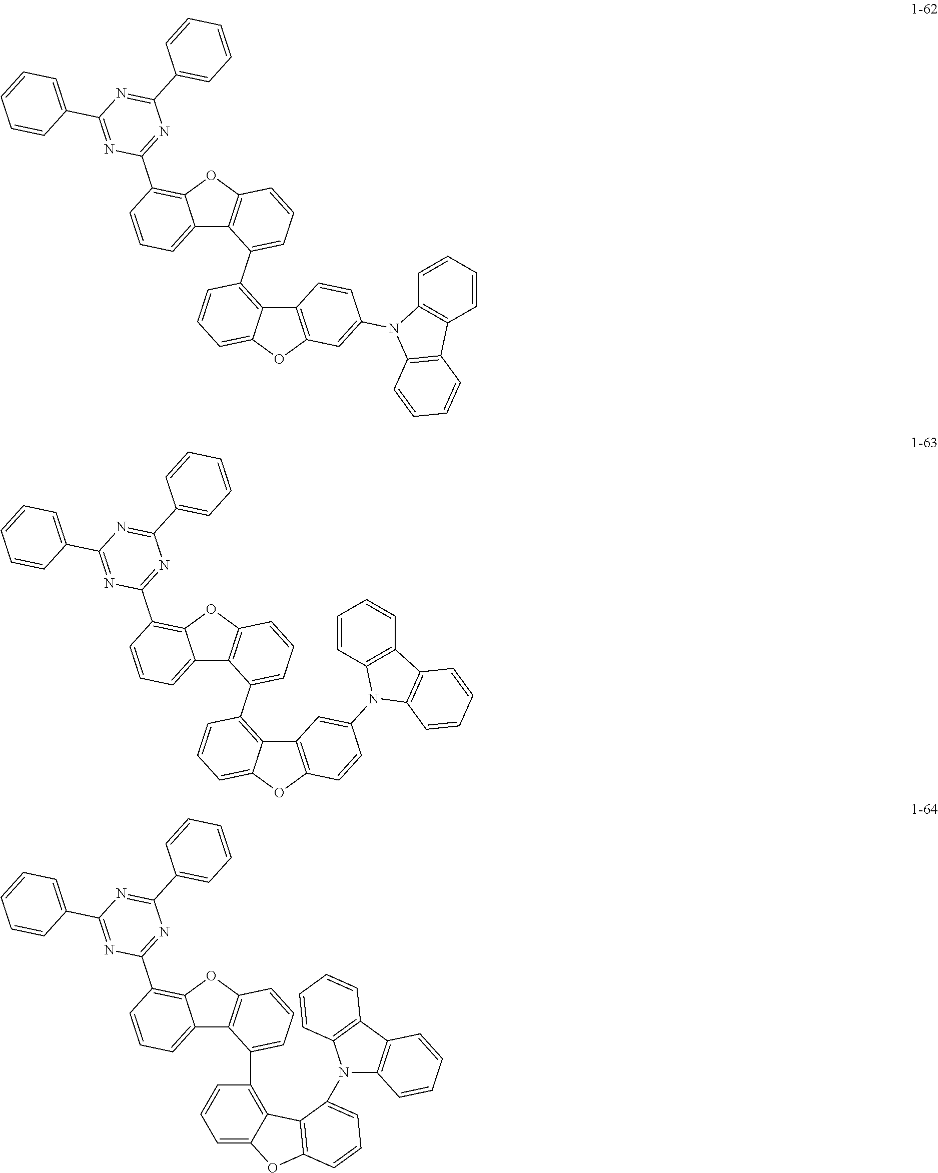

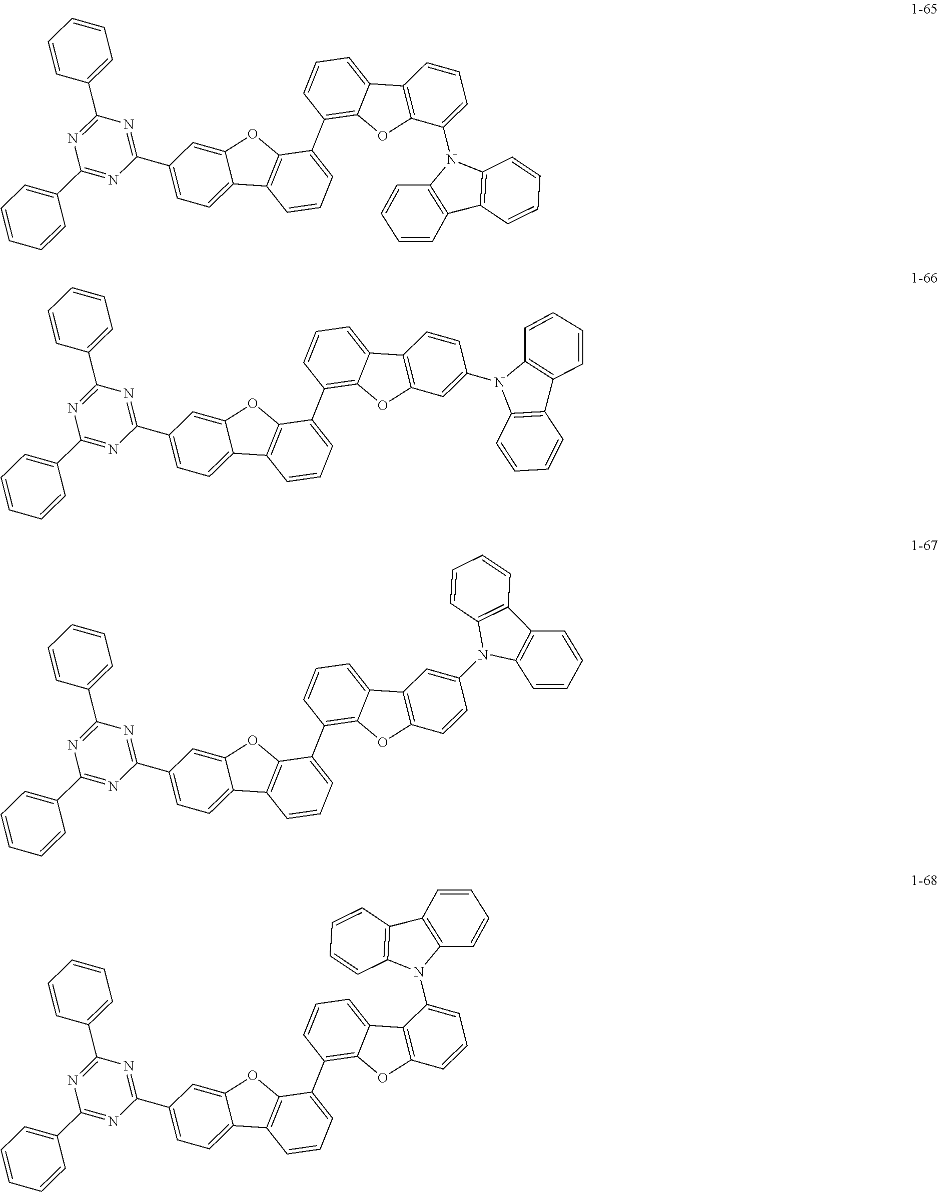 [SMILES: c1ccc(-c2nc(-c3ccccc3)nc(-c3ccc4c(c3)oc3c(-c5cccc6c5oc5c(-n7c8ccccc8c8ccccc87)cccc56)cccc34)n2)cc1 ; c1ccc(-c2nc(-c3ccccc3)nc(-c3ccc4c(c3)oc3c(-c5cccc6c5oc5cc(-n7c8ccccc8c8ccccc87)ccc56)cccc34)n2)cc1 ; c1ccc(-c2nc(-c3ccccc3)nc(-c3ccc4c(c3)oc3c(-c5cccc6c5oc5ccc(-n7c8ccccc8c8ccccc87)cc56)cccc34)n2)cc1 ; c1ccc(-c2nc(-c3ccccc3)nc(-c3ccc4c(c3)oc3c(-c5cccc6c5oc5cccc(-n7c8ccccc8c8ccccc87)c56)cccc34)n2)cc1]

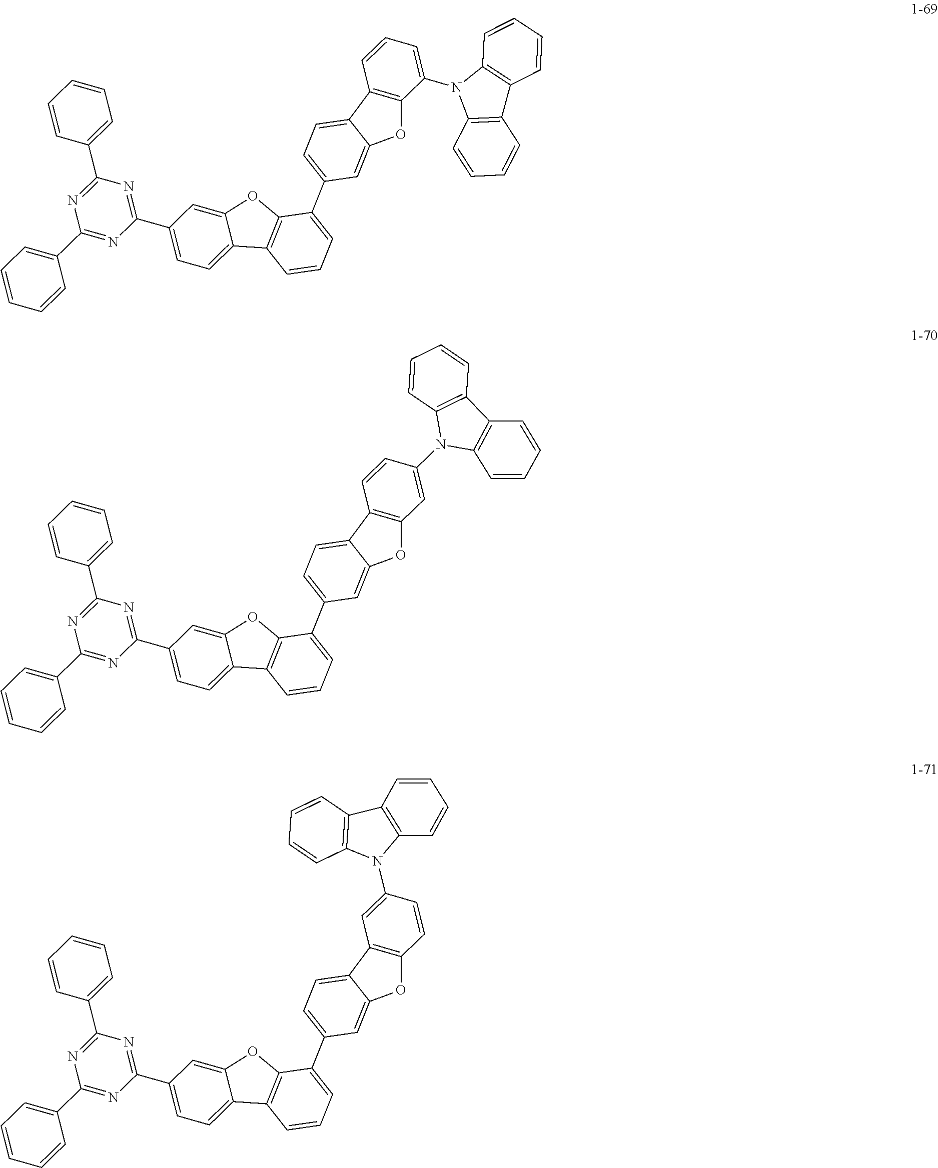 [SMILES: c1ccc(-c2nc(-c3ccccc3)nc(-c3ccc4c(c3)oc3c(-c5ccc6c(c5)oc5c(-n7c8ccccc8c8ccccc87)cccc56)cccc34)n2)cc1 ; c1ccc(-c2nc(-c3ccccc3)nc(-c3ccc4c(c3)oc3c(-c5ccc6c(c5)oc5cc(-n7c8ccccc8c8ccccc87)ccc56)cccc34)n2)cc1 ; c1ccc(-c2nc(-c3ccccc3)nc(-c3ccc4c(c3)oc3c(-c5ccc6c(c5)oc5ccc(-n7c8ccccc8c8ccccc87)cc56)cccc34)n2)cc1]

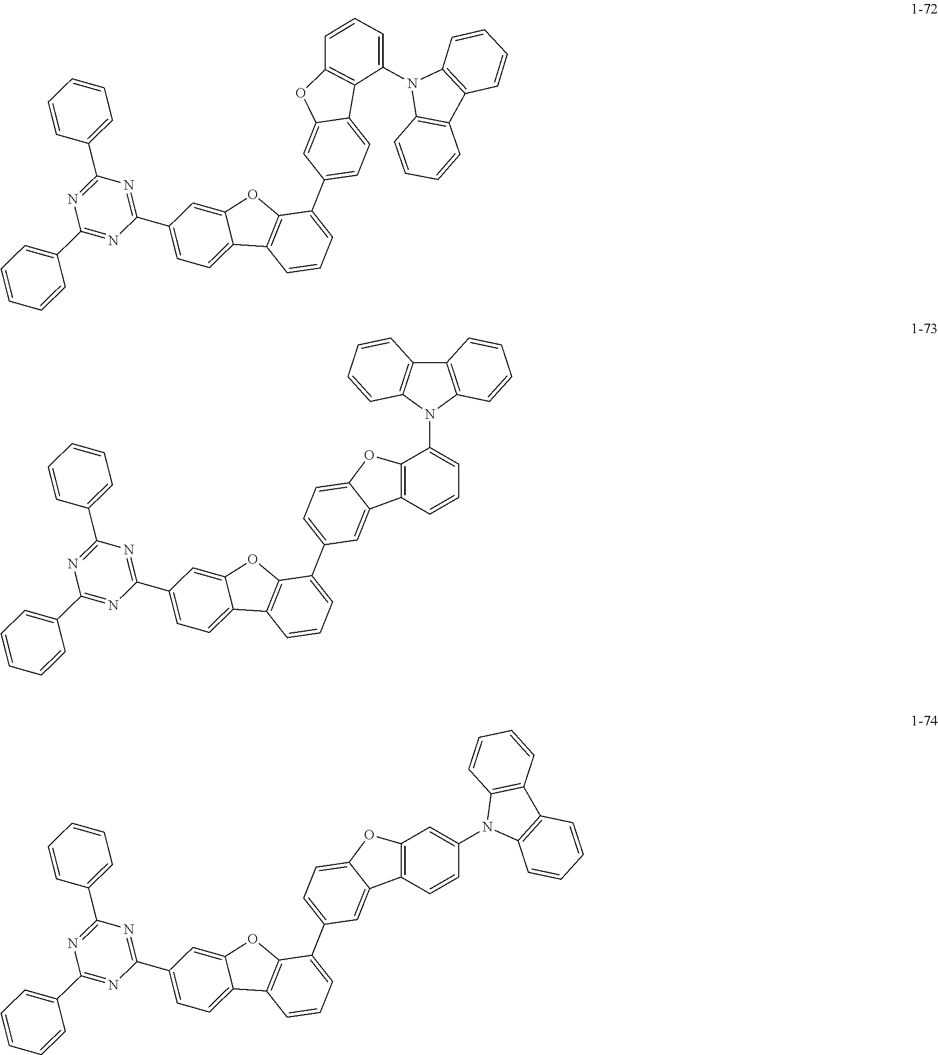 [SMILES: c1ccc(-c2nc(-c3ccccc3)nc(-c3ccc4c(c3)oc3c(-c5ccc6c(c5)oc5cccc(-n7c8ccccc8c8ccccc87)c56)cccc34)n2)cc1 ; c1ccc(-c2nc(-c3ccccc3)nc(-c3ccc4c(c3)oc3c(-c5ccc6oc7c(-n8c9ccccc9c9ccccc98)cccc7c6c5)cccc34)n2)cc1 ; c1ccc(-c2nc(-c3ccccc3)nc(-c3ccc4c(c3)oc3c(-c5ccc6oc7cc(-n8c9ccccc9c9ccccc98)ccc7c6c5)cccc34)n2)cc1]

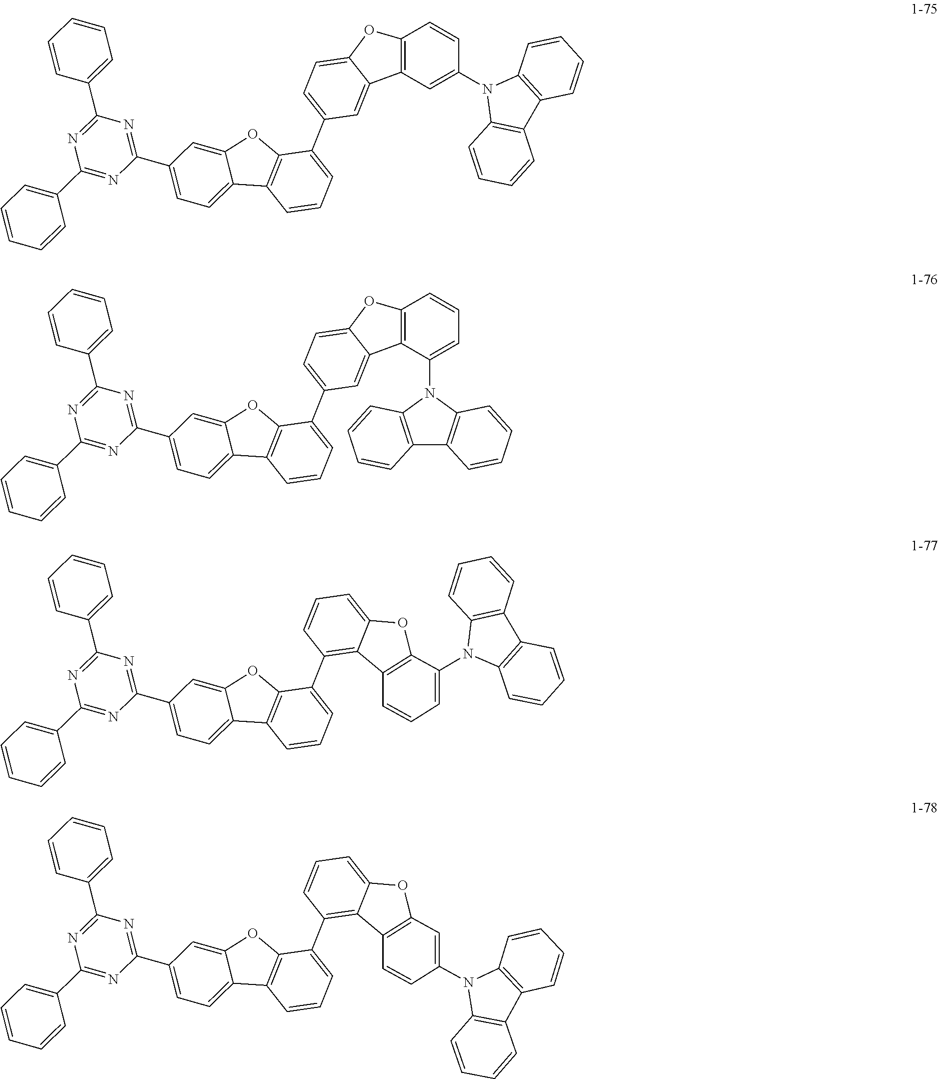

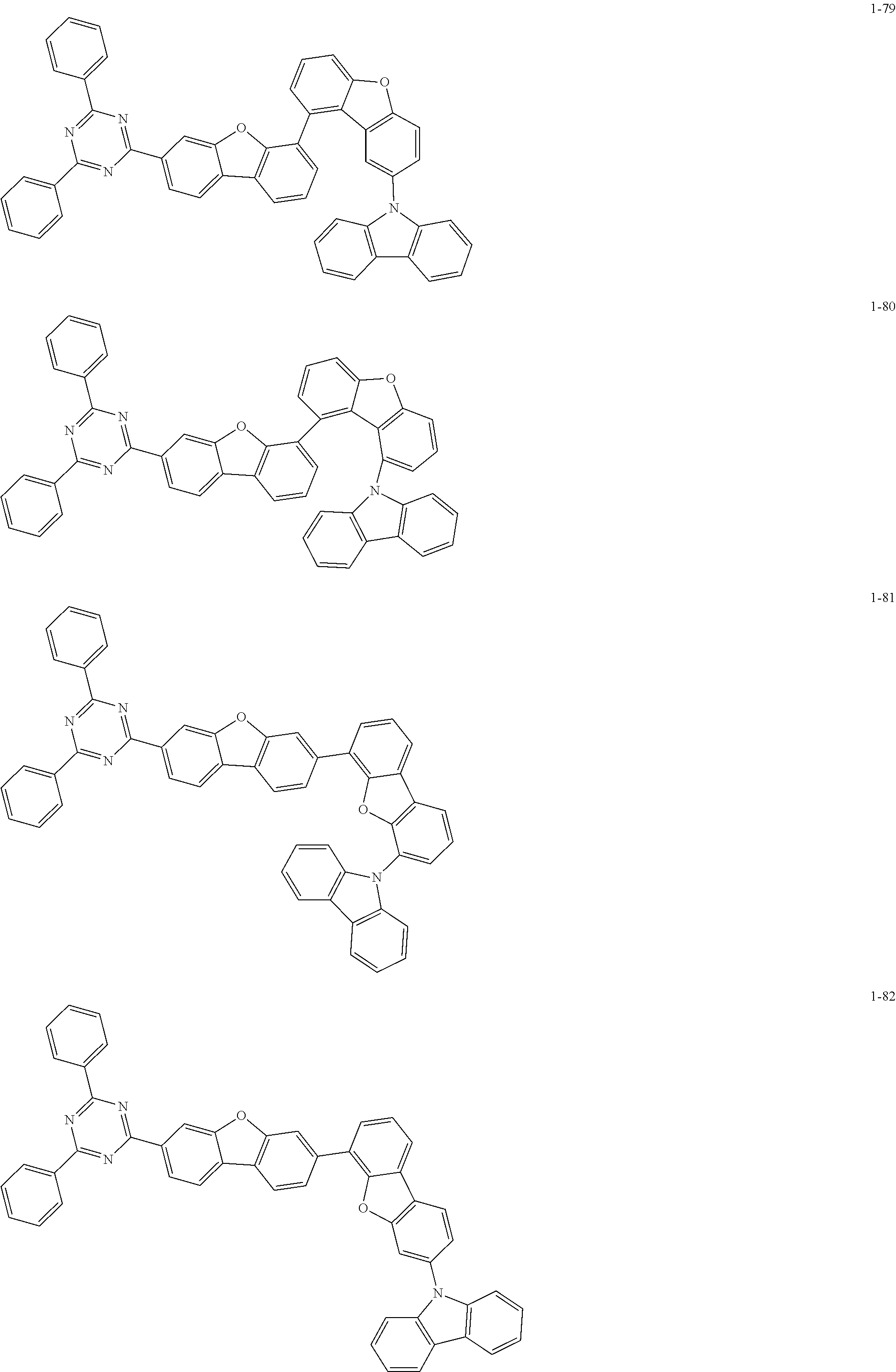 [SMILES: c1ccc(-c2nc(-c3ccccc3)nc(-c3ccc4c(c3)oc3c(-c5cccc6oc7ccc(-n8c9ccccc9c9ccccc98)cc7c56)cccc34)n2)cc1 ; c1ccc(-c2nc(-c3ccccc3)nc(-c3ccc4c(c3)oc3c(-c5cccc6oc7cccc(-n8c9ccccc9c9ccccc98)c7c56)cccc34)n2)cc1 ; c1ccc(-c2nc(-c3ccccc3)nc(-c3ccc4c(c3)oc3cc(-c5cccc6c5oc5c(-n7c8ccccc8c8ccccc87)cccc56)ccc34)n2)cc1 ; c1ccc(-c2nc(-c3ccccc3)nc(-c3ccc4c(c3)oc3cc(-c5cccc6c5oc5cc(-n7c8ccccc8c8ccccc87)ccc56)ccc34)n2)cc1]

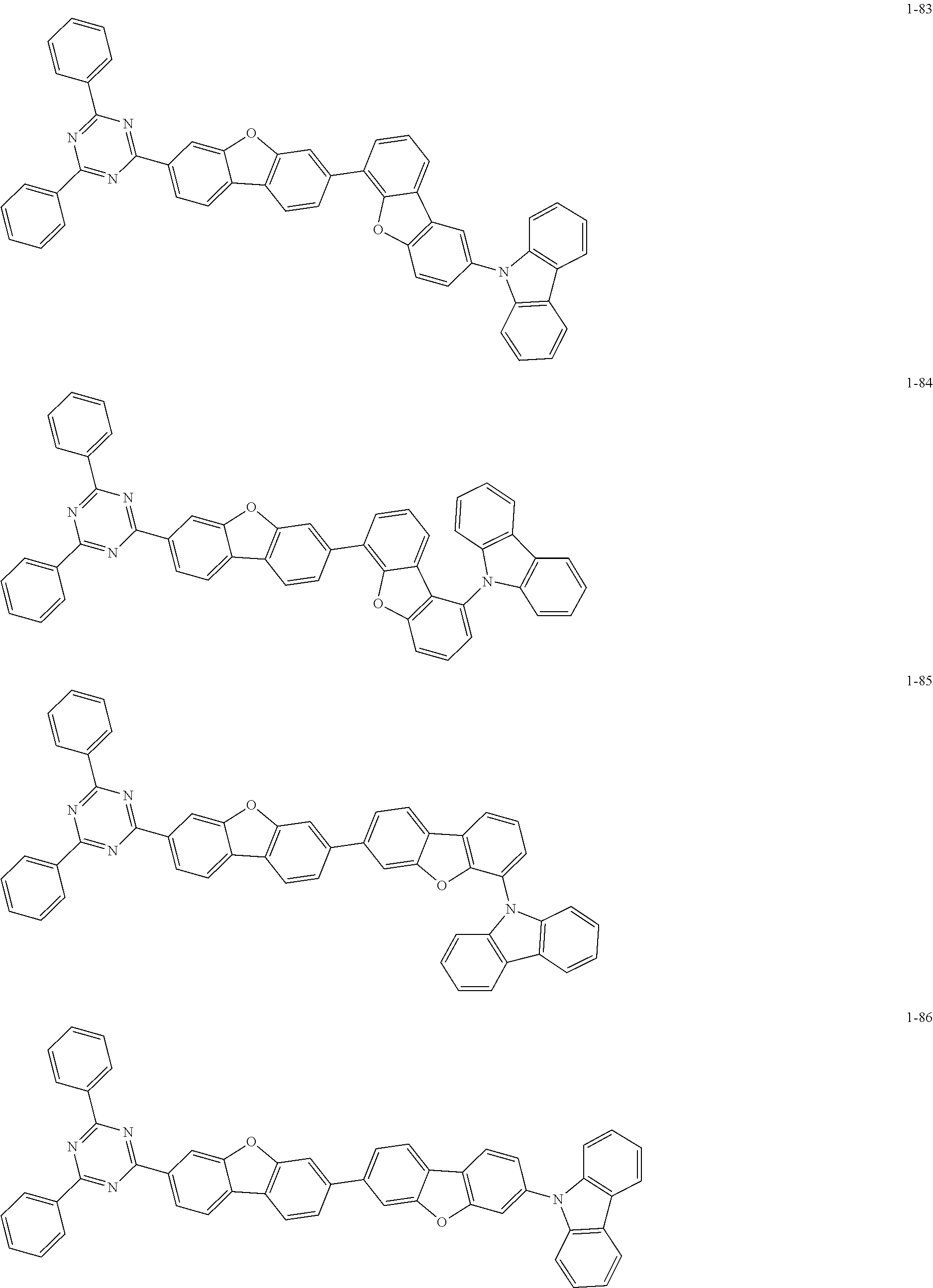 [SMILES: c1ccc(-c2nc(-c3ccccc3)nc(-c3ccc4c(c3)oc3cc(-c5cccc6c5oc5ccc(-n7c8ccccc8c8ccccc87)cc56)ccc34)n2)cc1 ; c1ccc(-c2nc(-c3ccccc3)nc(-c3ccc4c(c3)oc3cc(-c5cccc6c5oc5cccc(-n7c8ccccc8c8ccccc87)c56)ccc34)n2)cc1 ; c1ccc(-c2nc(-c3ccccc3)nc(-c3ccc4c(c3)oc3cc(-c5ccc6c(c5)oc5c(-n7c8ccccc8c8ccccc87)cccc56)ccc34)n2)cc1 ; c1ccc(-c2nc(-c3ccccc3)nc(-c3ccc4c(c3)oc3cc(-c5ccc6c(c5)oc5cc(-n7c8ccccc8c8ccccc87)ccc56)ccc34)n2)cc1]

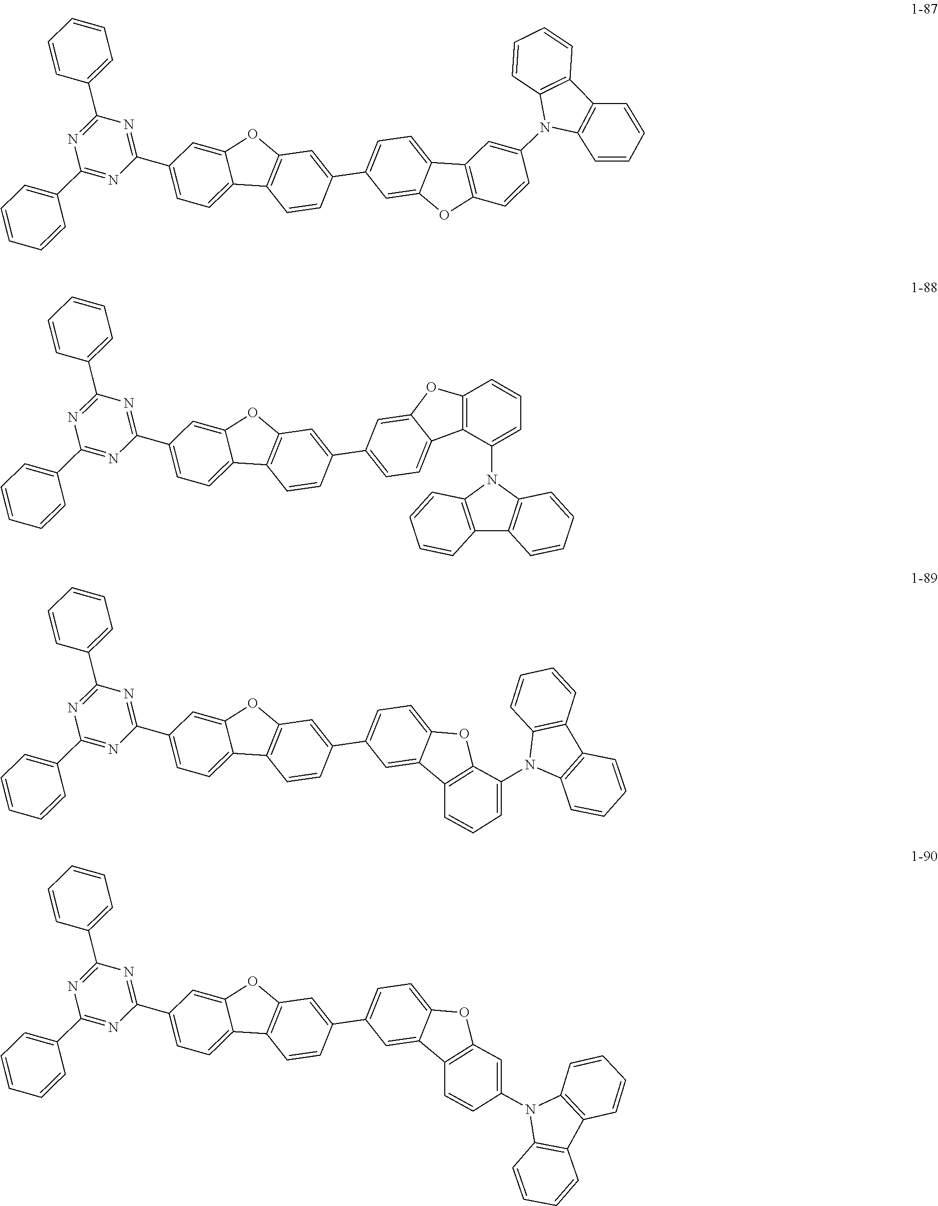 [SMILES: c1ccc(-c2nc(-c3ccccc3)nc(-c3ccc4c(c3)oc3cc(-c5ccc6c(c5)oc5ccc(-n7c8ccccc8c8ccccc87)cc56)ccc34)n2)cc1 ; c1ccc(-c2nc(-c3ccccc3)nc(-c3ccc4c(c3)oc3cc(-c5ccc6c(c5)oc5cccc(-n7c8ccccc8c8ccccc87)c56)ccc34)n2)cc1 ; c1ccc(-c2nc(-c3ccccc3)nc(-c3ccc4c(c3)oc3cc(-c5ccc6oc7c(-n8c9ccccc9c9ccccc98)cccc7c6c5)ccc34)n2)cc1 ; c1ccc(-c2nc(-c3ccccc3)nc(-c3ccc4c(c3)oc3cc(-c5ccc6oc7cc(-n8c9ccccc9c9ccccc98)ccc7c6c5)ccc34)n2)cc1]

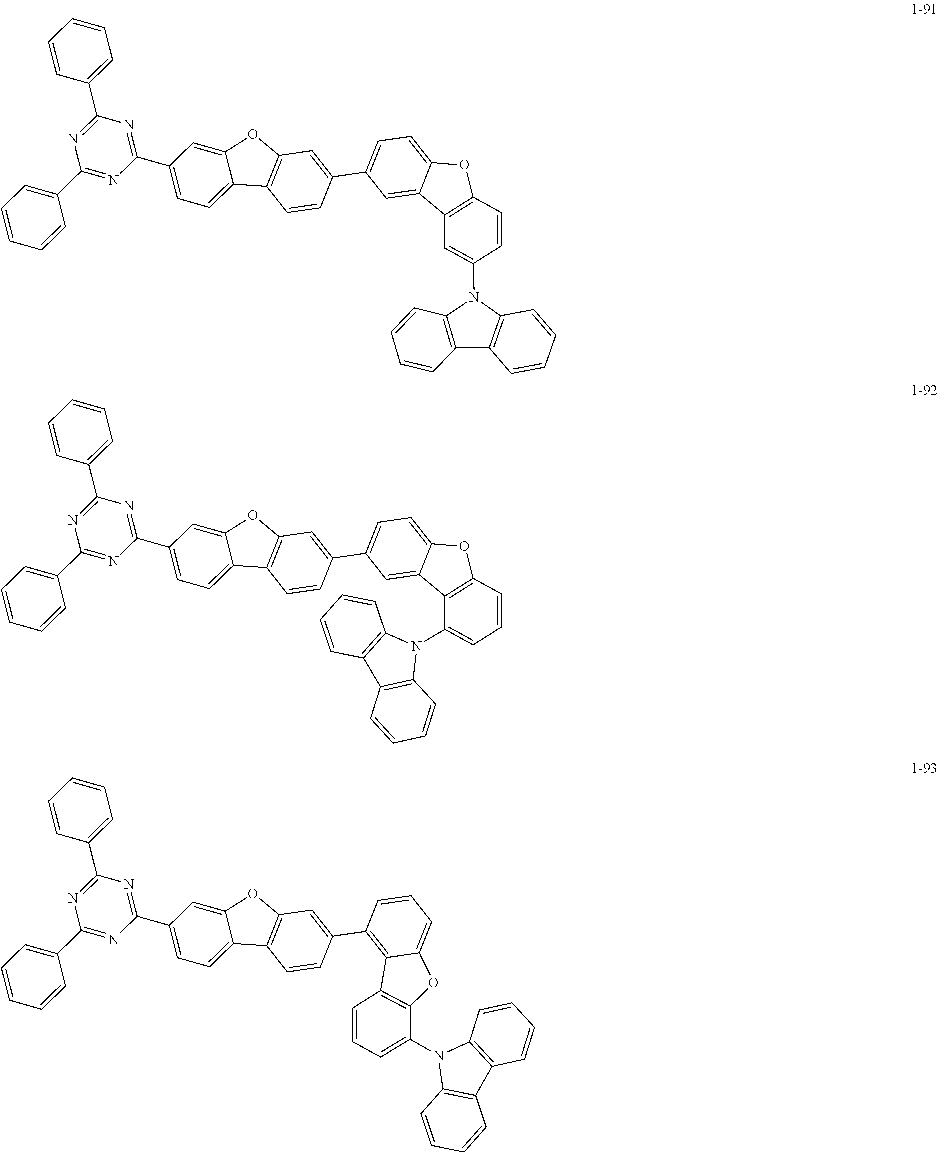 [SMILES: c1ccc(-c2nc(-c3ccccc3)nc(-c3ccc4c(c3)oc3cc(-c5ccc6oc7ccc(-n8c9ccccc9c9ccccc98)cc7c6c5)ccc34)n2)cc1 ; c1ccc(-c2nc(-c3ccccc3)nc(-c3ccc4c(c3)oc3cc(-c5ccc6oc7cccc(-n8c9ccccc9c9ccccc98)c7c6c5)ccc34)n2)cc1 ; c1ccc(-c2nc(-c3ccccc3)nc(-c3ccc4c(c3)oc3cc(-c5cccc6oc7c(-n8c9ccccc9c9ccccc98)cccc7c56)ccc34)n2)cc1]

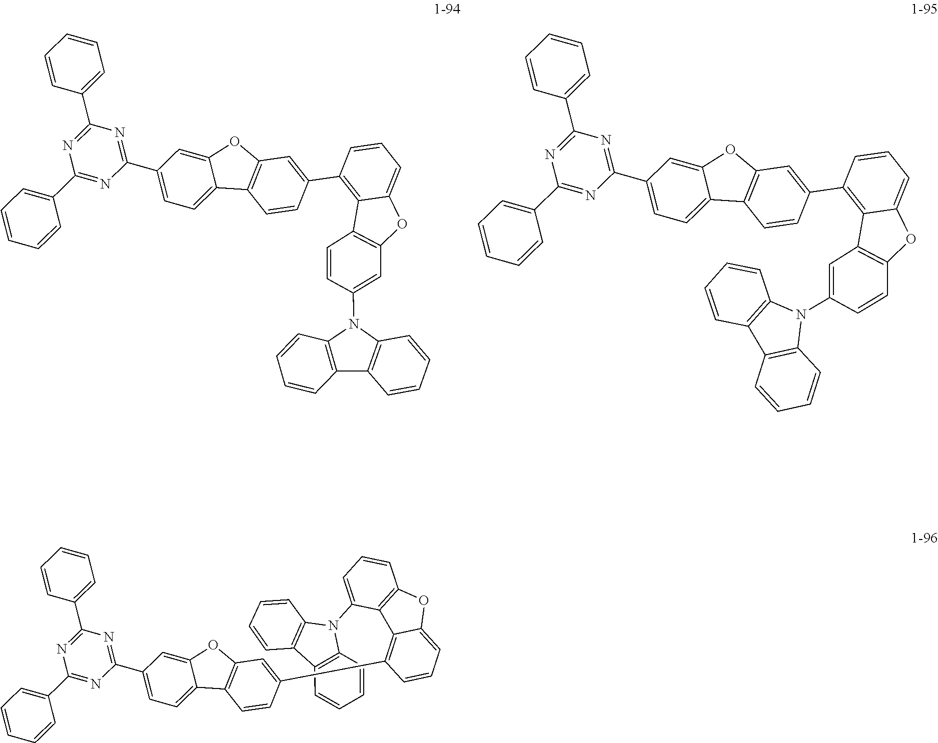 [SMILES: c1ccc(-c2nc(-c3ccccc3)nc(-c3ccc4c(c3)oc3cc(-c5cccc6oc7cc(-n8c9ccccc9c9ccccc98)ccc7c56)ccc34)n2)cc1 ; c1ccc(-c2nc(-c3ccccc3)nc(-c3ccc4c(c3)oc3cc(-c5cccc6oc7ccc(-n8c9ccccc9c9ccccc98)cc7c56)ccc34)n2)cc1 ; c1ccc(-c2nc(-c3ccccc3)nc(-c3ccc4c(c3)oc3cc(-c5cccc6oc7cccc(-n8c9ccccc9c9ccccc98)c7c56)ccc34)n2)cc1]

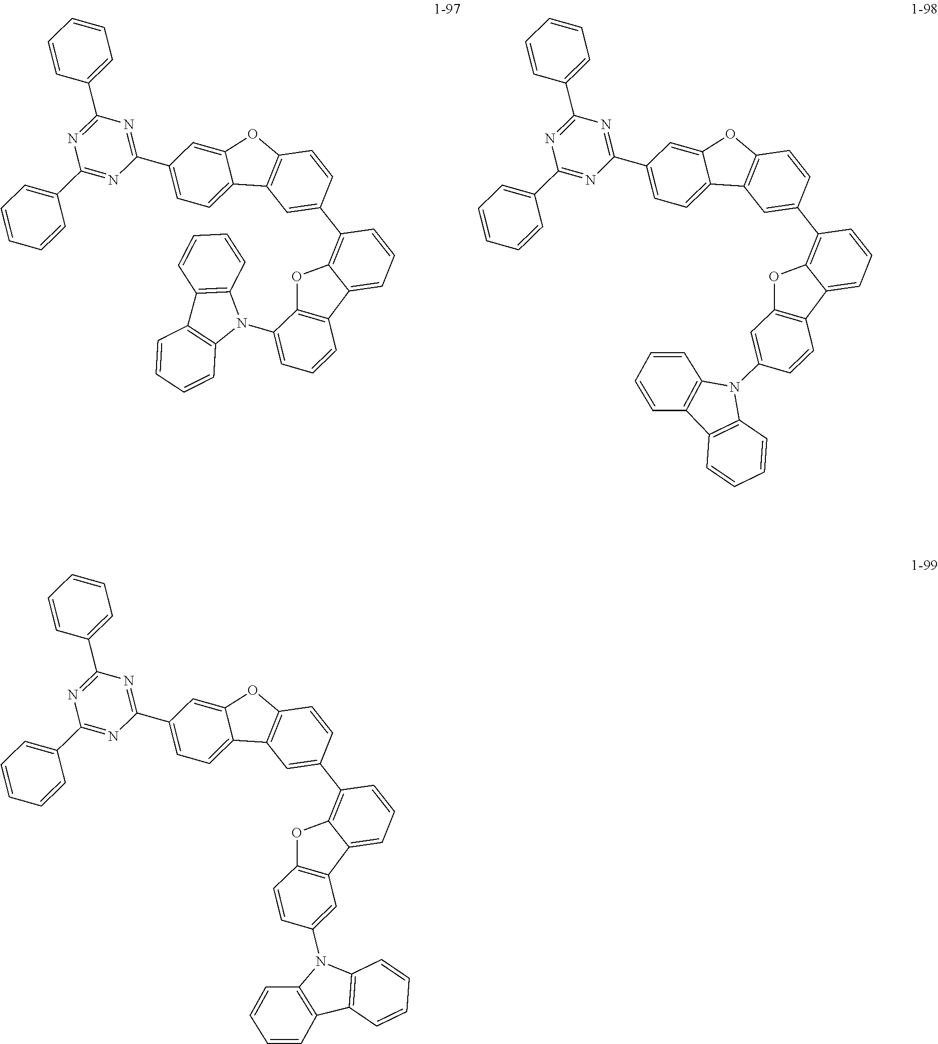 [SMILES: c1ccc(-c2nc(-c3ccccc3)nc(-c3ccc4c(c3)oc3ccc(-c5cccc6c5oc5c(-n7c8ccccc8c8ccccc87)cccc56)cc34)n2)cc1 ; c1ccc(-c2nc(-c3ccccc3)nc(-c3ccc4c(c3)oc3ccc(-c5cccc6c5oc5cc(-n7c8ccccc8c8ccccc87)ccc56)cc34)n2)cc1 ; c1ccc(-c2nc(-c3ccccc3)nc(-c3ccc4c(c3)oc3ccc(-c5cccc6c5oc5ccc(-n7c8ccccc8c8ccccc87)cc56)cc34)n2)cc1]

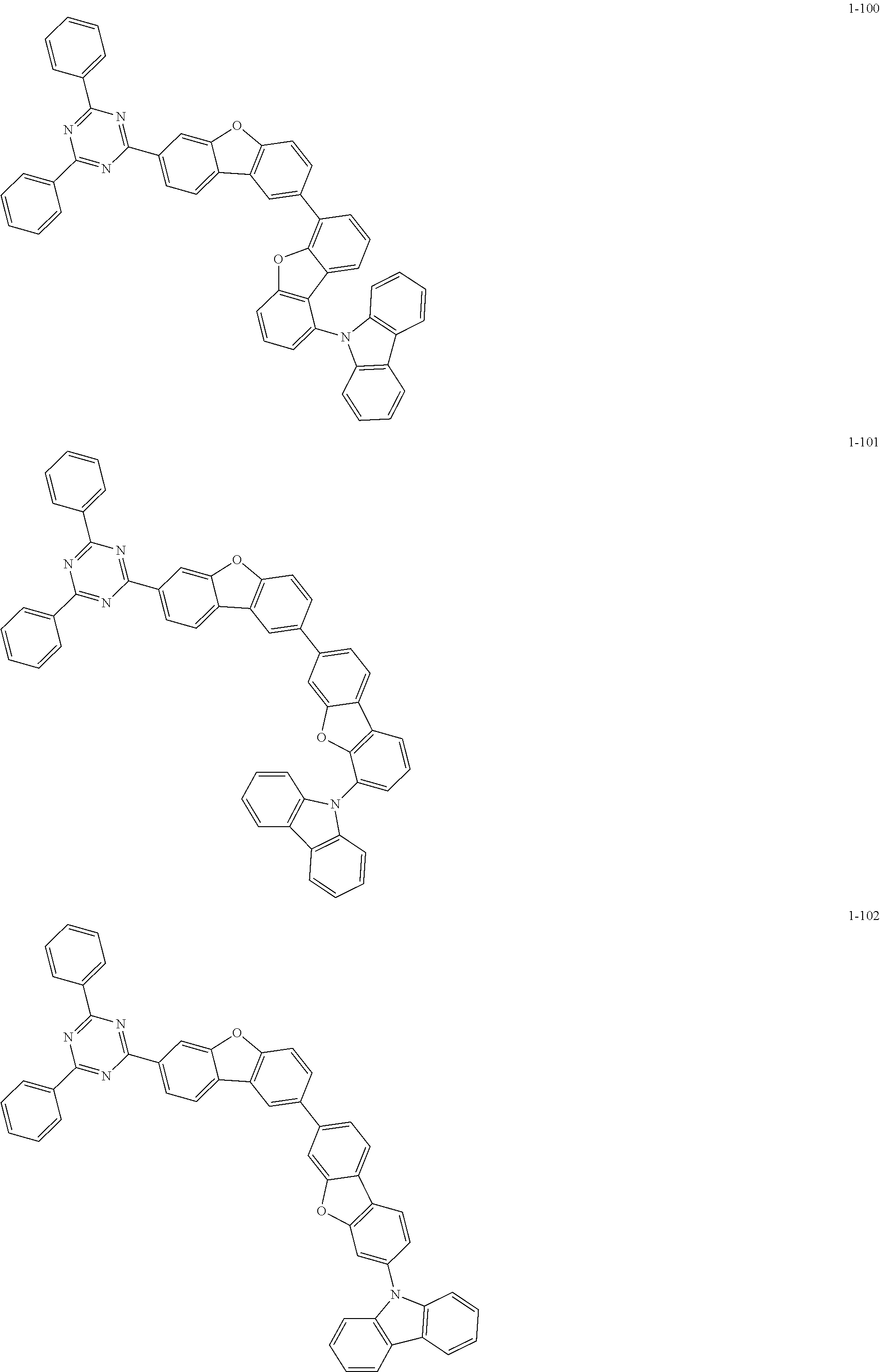 [SMILES: c1ccc(-c2nc(-c3ccccc3)nc(-c3ccc4c(c3)oc3ccc(-c5cccc6c5oc5cccc(-n7c8ccccc8c8ccccc87)c56)cc34)n2)cc1 ; c1ccc(-c2nc(-c3ccccc3)nc(-c3ccc4c(c3)oc3ccc(-c5ccc6c(c5)oc5c(-n7c8ccccc8c8ccccc87)cccc56)cc34)n2)cc1 ; c1ccc(-c2nc(-c3ccccc3)nc(-c3ccc4c(c3)oc3ccc(-c5ccc6c(c5)oc5cc(-n7c8ccccc8c8ccccc87)ccc56)cc34)n2)cc1]

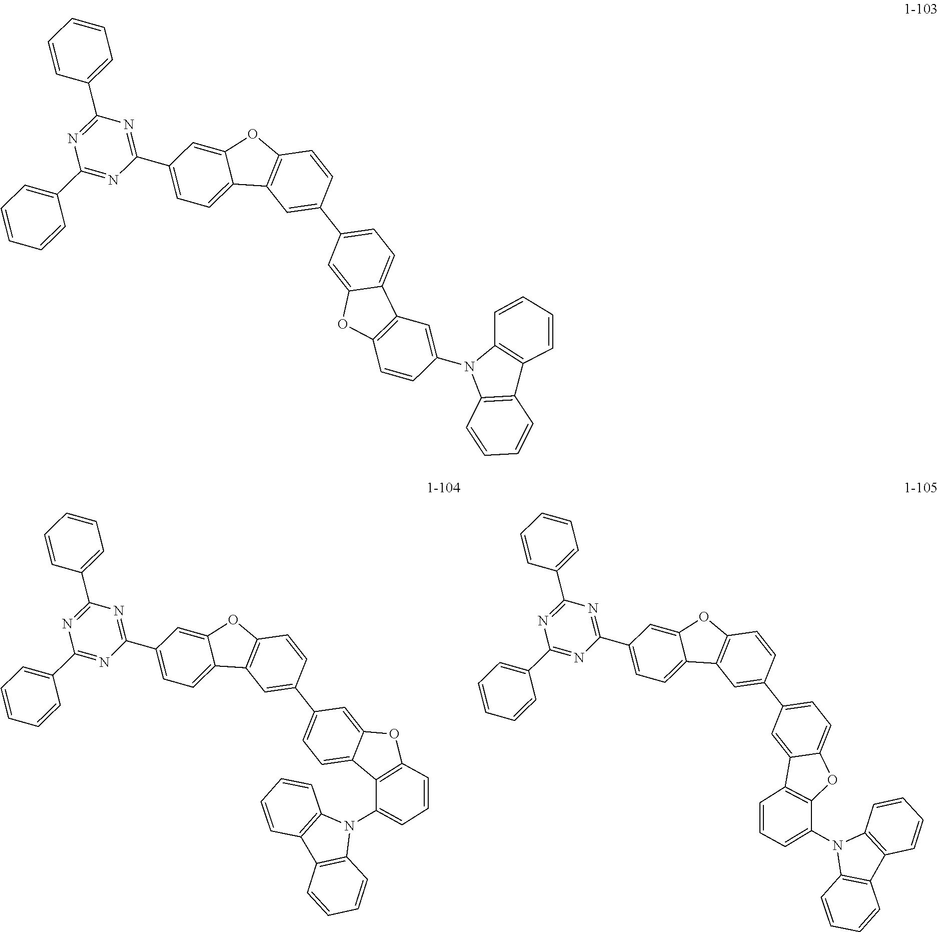 [SMILES: c1ccc(-c2nc(-c3ccccc3)nc(-c3ccc4c(c3)oc3ccc(-c5ccc6c(c5)oc5ccc(-n7c8ccccc8c8ccccc87)cc56)cc34)n2)cc1 ; c1ccc(-c2nc(-c3ccccc3)nc(-c3ccc4c(c3)oc3ccc(-c5ccc6c(c5)oc5cccc(-n7c8ccccc8c8ccccc87)c56)cc34)n2)cc1 ; c1ccc(-c2nc(-c3ccccc3)nc(-c3ccc4c(c3)oc3ccc(-c5ccc6oc7c(-n8c9ccccc9c9ccccc98)cccc7c6c5)cc34)n2)cc1]

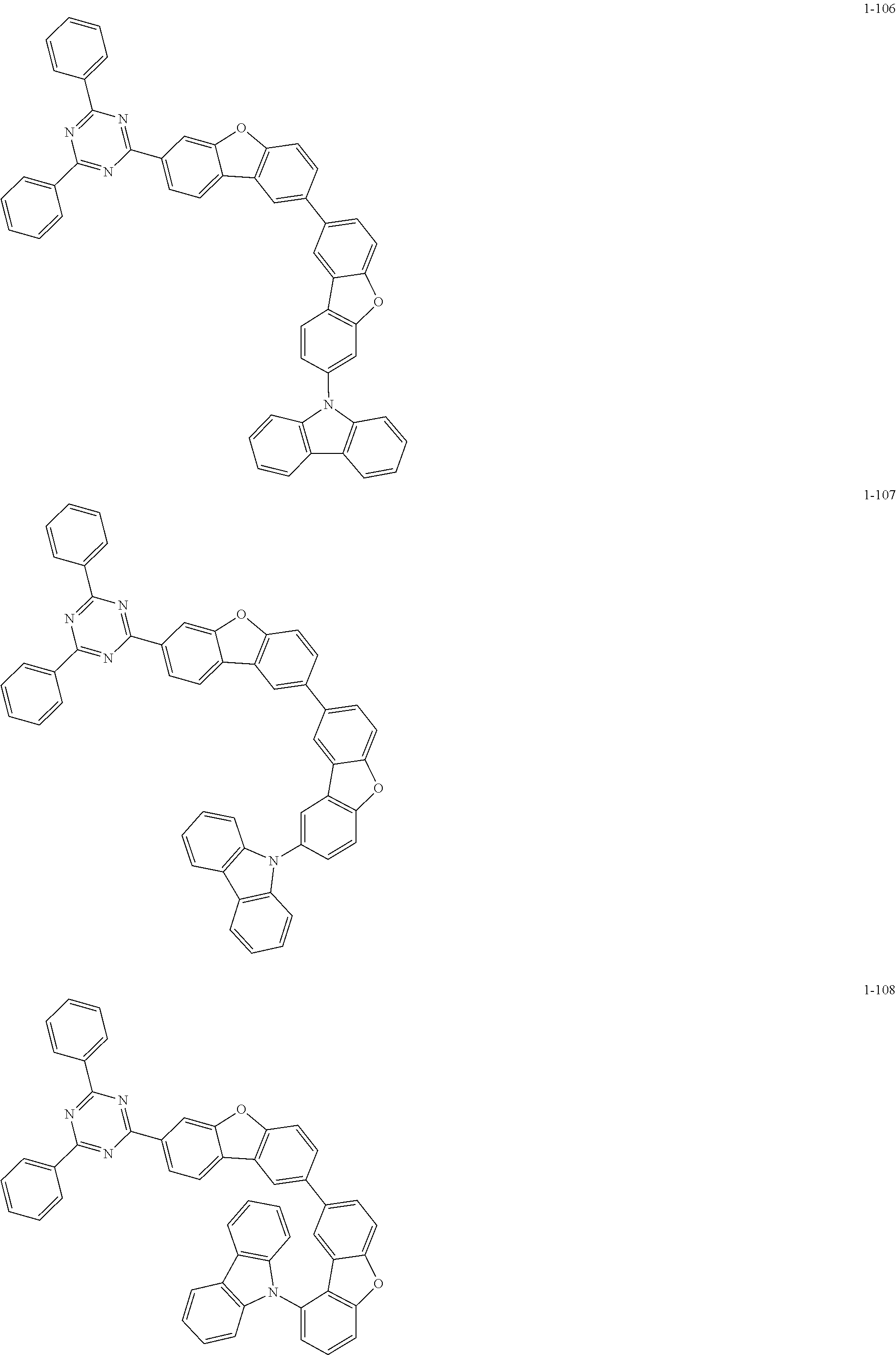 [SMILES: c1ccc(-c2nc(-c3ccccc3)nc(-c3ccc4c(c3)oc3ccc(-c5ccc6oc7cc(-n8c9ccccc9c9ccccc98)ccc7c6c5)cc34)n2)cc1 ; c1ccc(-c2nc(-c3ccccc3)nc(-c3ccc4c(c3)oc3ccc(-c5ccc6oc7ccc(-n8c9ccccc9c9ccccc98)cc7c6c5)cc34)n2)cc1 ; c1ccc(-c2nc(-c3ccccc3)nc(-c3ccc4c(c3)oc3ccc(-c5ccc6oc7cccc(-n8c9ccccc9c9ccccc98)c7c6c5)cc34)n2)cc1]

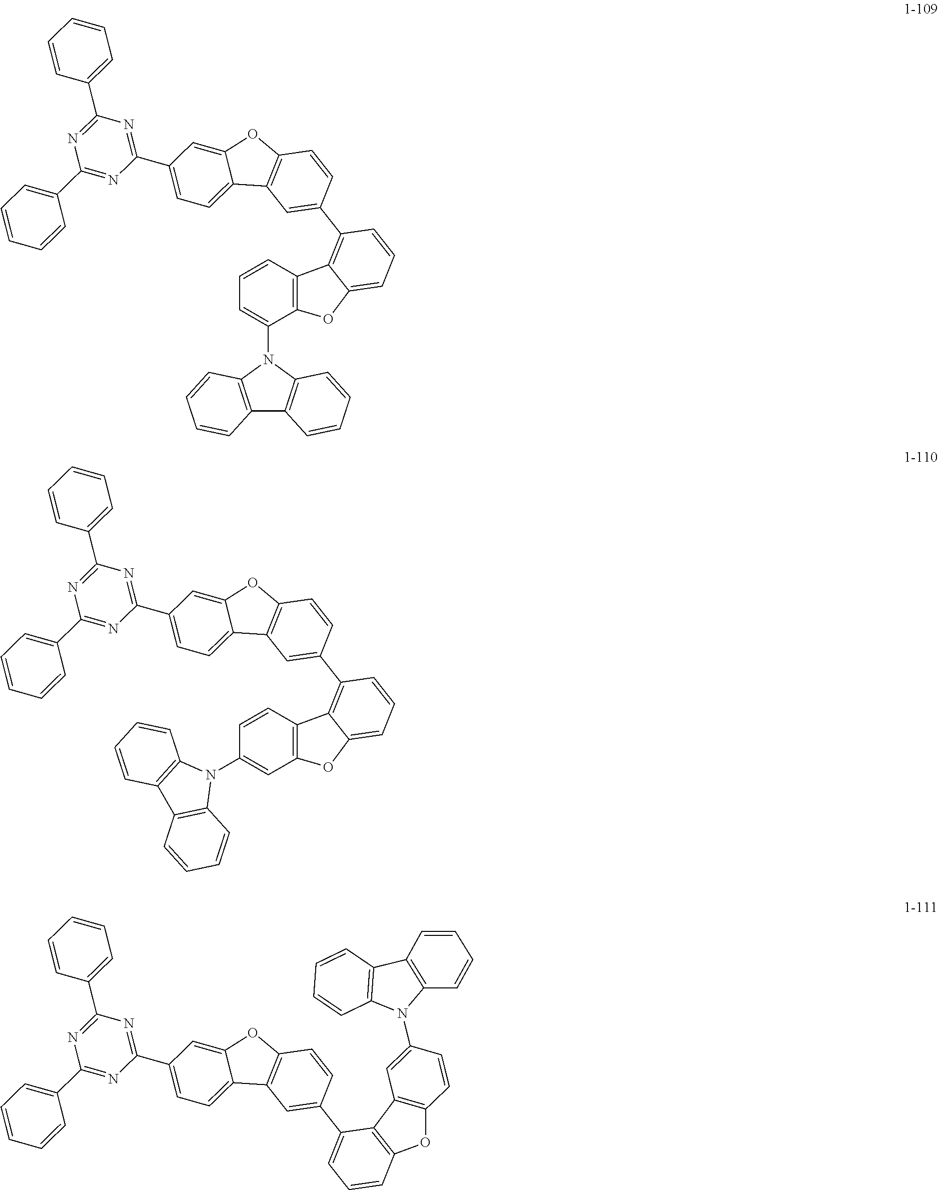 [SMILES: c1ccc(-c2nc(-c3ccccc3)nc(-c3ccc4c(c3)oc3ccc(-c5cccc6oc7c(-n8c9ccccc9c9ccccc98)cccc7c56)cc34)n2)cc1 ; c1ccc(-c2nc(-c3ccccc3)nc(-c3ccc4c(c3)oc3ccc(-c5cccc6oc7cc(-n8c9ccccc9c9ccccc98)ccc7c56)cc34)n2)cc1 ; c1ccc(-c2nc(-c3ccccc3)nc(-c3ccc4c(c3)oc3ccc(-c5cccc6oc7ccc(-n8c9ccccc9c9ccccc98)cc7c56)cc34)n2)cc1]

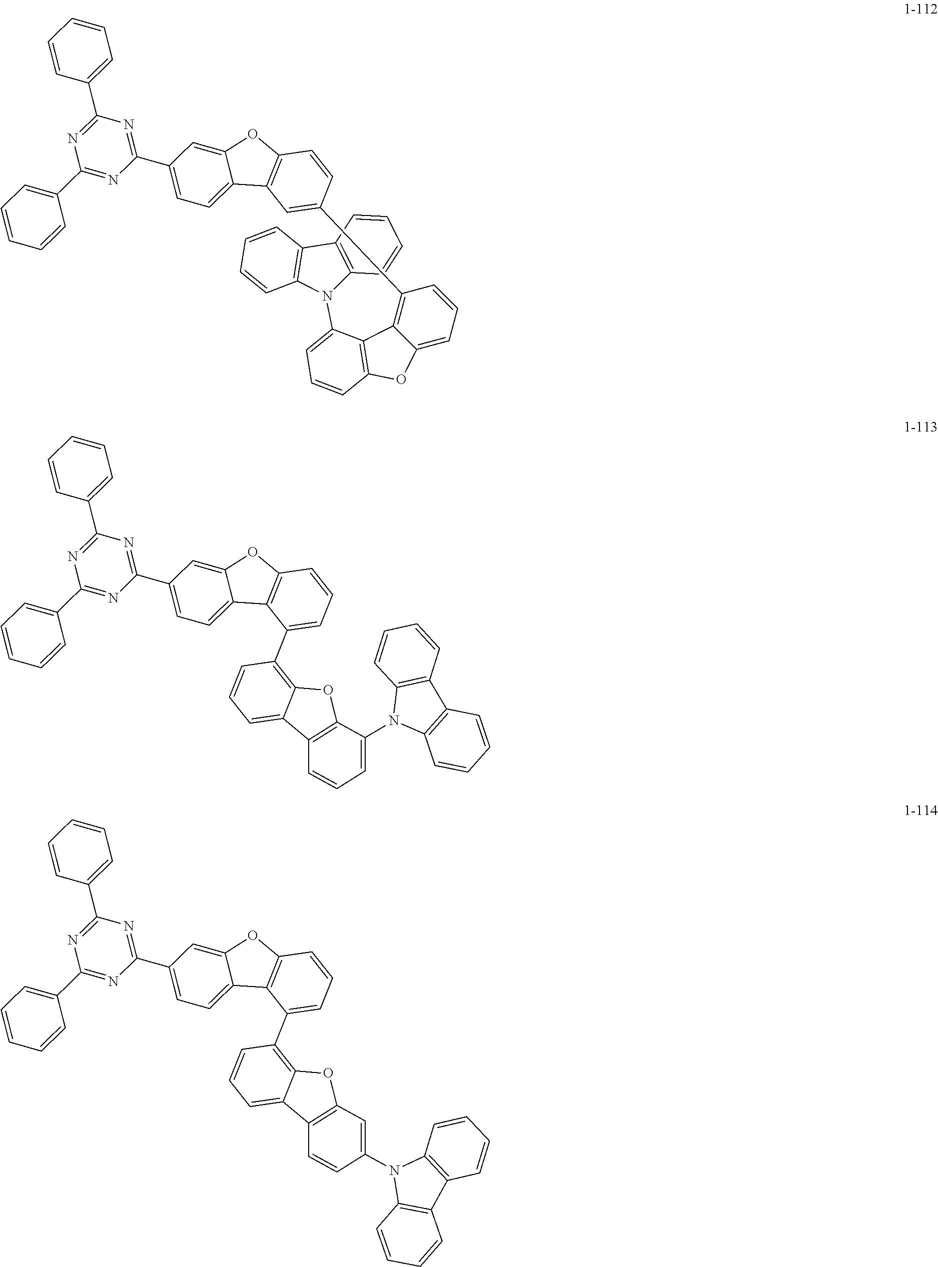 [SMILES: c1ccc(-c2nc(-c3ccccc3)nc(-c3ccc4c(c3)oc3ccc(-c5cccc6oc7cccc(-n8c9ccccc9c9ccccc98)c7c56)cc34)n2)cc1 ; c1ccc(-c2nc(-c3ccccc3)nc(-c3ccc4c(c3)oc3cccc(-c5cccc6c5oc5c(-n7c8ccccc8c8ccccc87)cccc56)c34)n2)cc1 ; c1ccc(-c2nc(-c3ccccc3)nc(-c3ccc4c(c3)oc3cccc(-c5cccc6c5oc5cc(-n7c8ccccc8c8ccccc87)ccc56)c34)n2)cc1]

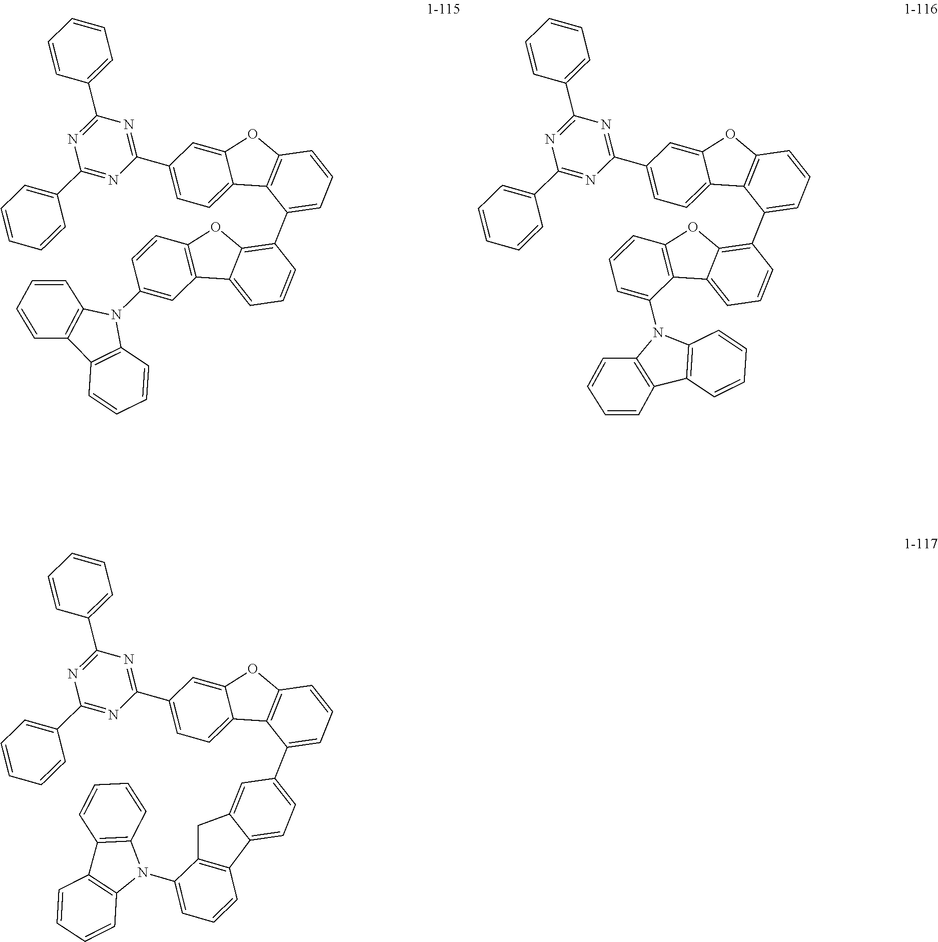 [SMILES: c1ccc(-c2nc(-c3ccccc3)nc(-c3ccc4c(c3)oc3cccc(-c5cccc6c5oc5ccc(-n7c8ccccc8c8ccccc87)cc56)c34)n2)cc1 ; c1ccc(-c2nc(-c3ccccc3)nc(-c3ccc4c(c3)oc3cccc(-c5cccc6c5oc5cccc(-n7c8ccccc8c8ccccc87)c56)c34)n2)cc1 ; c1ccc(-c2nc(-c3ccccc3)nc(-c3ccc4c(c3)oc3cccc(-c5ccc6c(c5)Cc5c-6cccc5-n5c6ccccc6c6ccccc65)c34)n2)cc1]

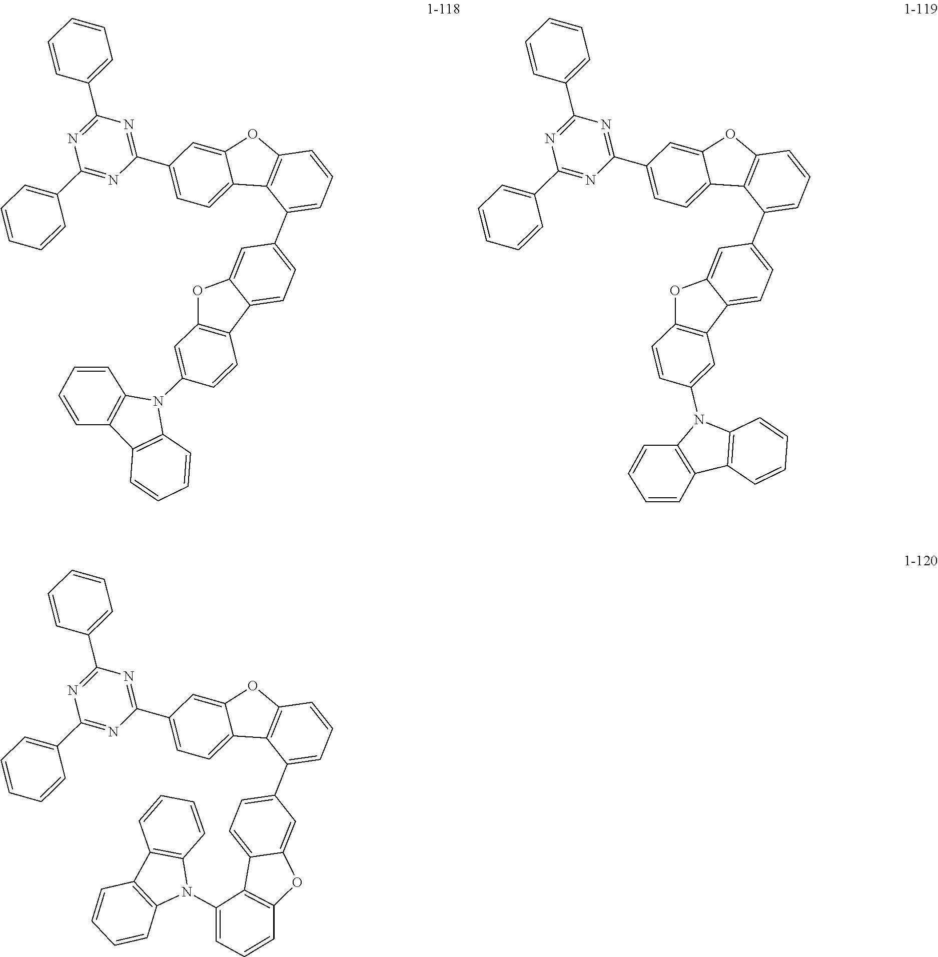 [SMILES: c1ccc(-c2nc(-c3ccccc3)nc(-c3ccc4c(c3)oc3cccc(-c5ccc6c(c5)oc5cc(-n7c8ccccc8c8ccccc87)ccc56)c34)n2)cc1 ; c1ccc(-c2nc(-c3ccccc3)nc(-c3ccc4c(c3)oc3cccc(-c5ccc6c(c5)oc5ccc(-n7c8ccccc8c8ccccc87)cc56)c34)n2)cc1 ; c1ccc(-c2nc(-c3ccccc3)nc(-c3ccc4c(c3)oc3cccc(-c5ccc6c(c5)oc5cccc(-n7c8ccccc8c8ccccc87)c56)c34)n2)cc1]

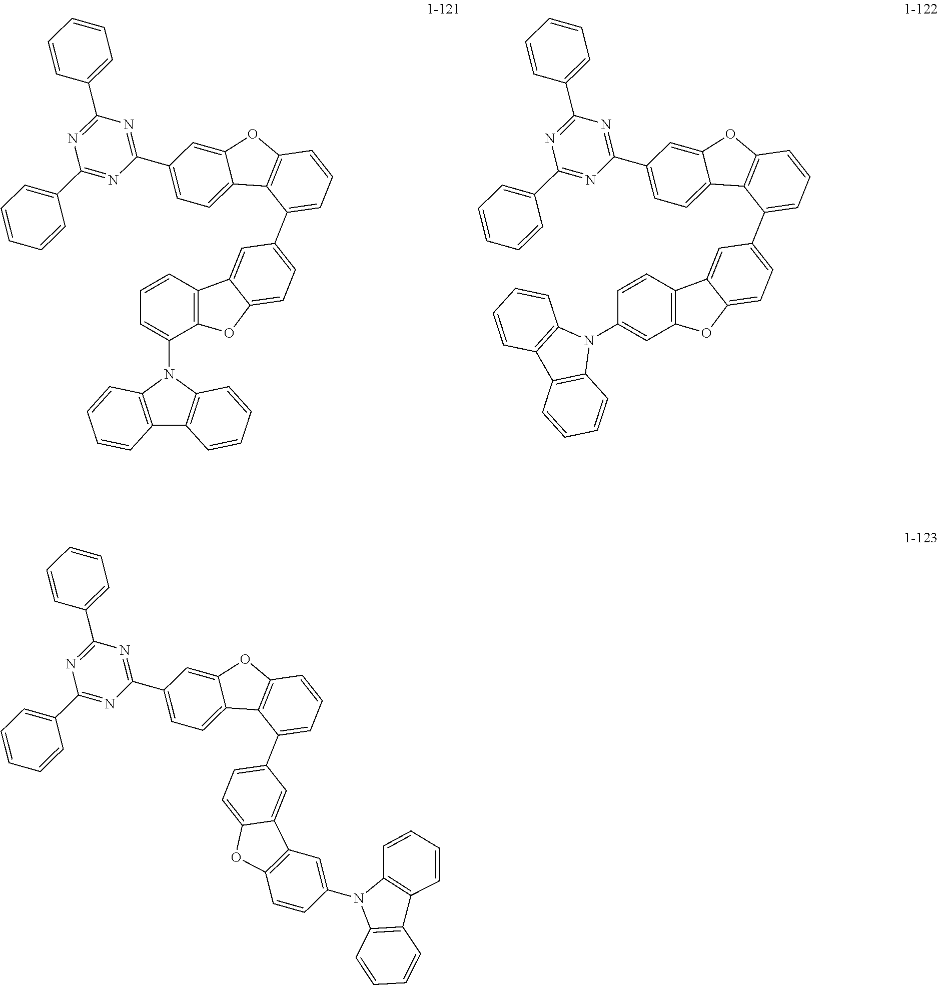 [SMILES: c1ccc(-c2nc(-c3ccccc3)nc(-c3ccc4c(c3)oc3cccc(-c5ccc6oc7c(-n8c9ccccc9c9ccccc98)cccc7c6c5)c34)n2)cc1 ; c1ccc(-c2nc(-c3ccccc3)nc(-c3ccc4c(c3)oc3cccc(-c5ccc6oc7cc(-n8c9ccccc9c9ccccc98)ccc7c6c5)c34)n2)cc1 ; c1ccc(-c2nc(-c3ccccc3)nc(-c3ccc4c(c3)oc3cccc(-c5ccc6oc7ccc(-n8c9ccccc9c9ccccc98)cc7c6c5)c34)n2)cc1]

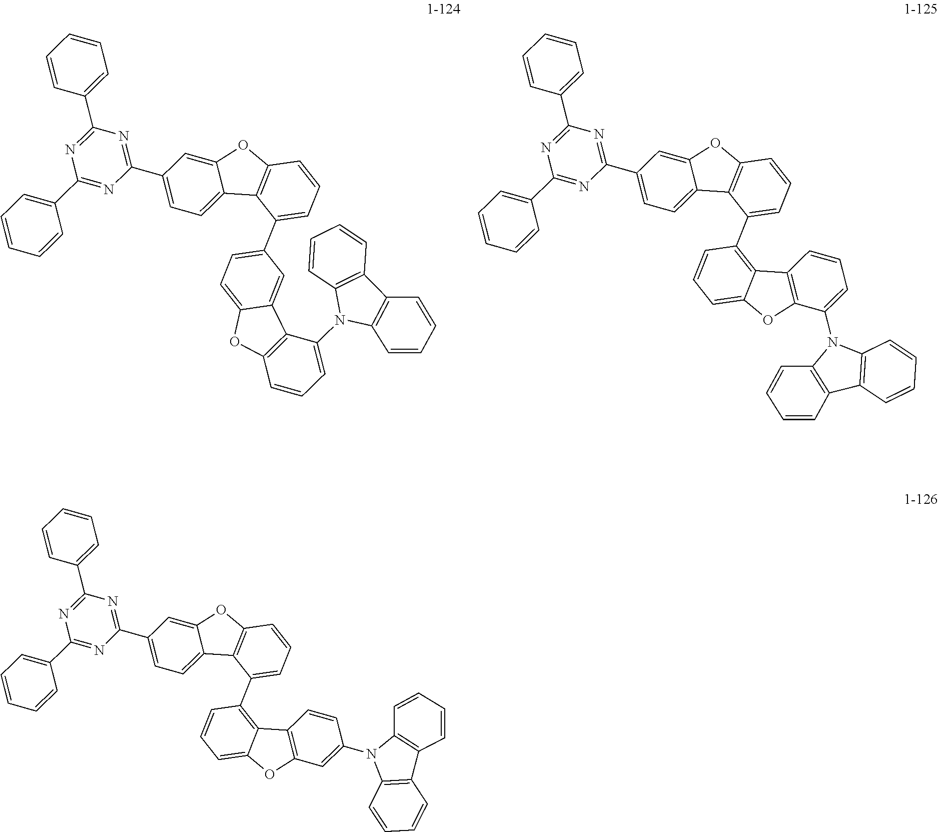 [SMILES: c1ccc(-c2nc(-c3ccccc3)nc(-c3ccc4c(c3)oc3cccc(-c5ccc6oc7cccc(-n8c9ccccc9c9ccccc98)c7c6c5)c34)n2)cc1 ; c1ccc(-c2nc(-c3ccccc3)nc(-c3ccc4c(c3)oc3cccc(-c5cccc6oc7c(-n8c9ccccc9c9ccccc98)cccc7c56)c34)n2)cc1 ; c1ccc(-c2nc(-c3ccccc3)nc(-c3ccc4c(c3)oc3cccc(-c5cccc6oc7cc(-n8c9ccccc9c9ccccc98)ccc7c56)c34)n2)cc1]

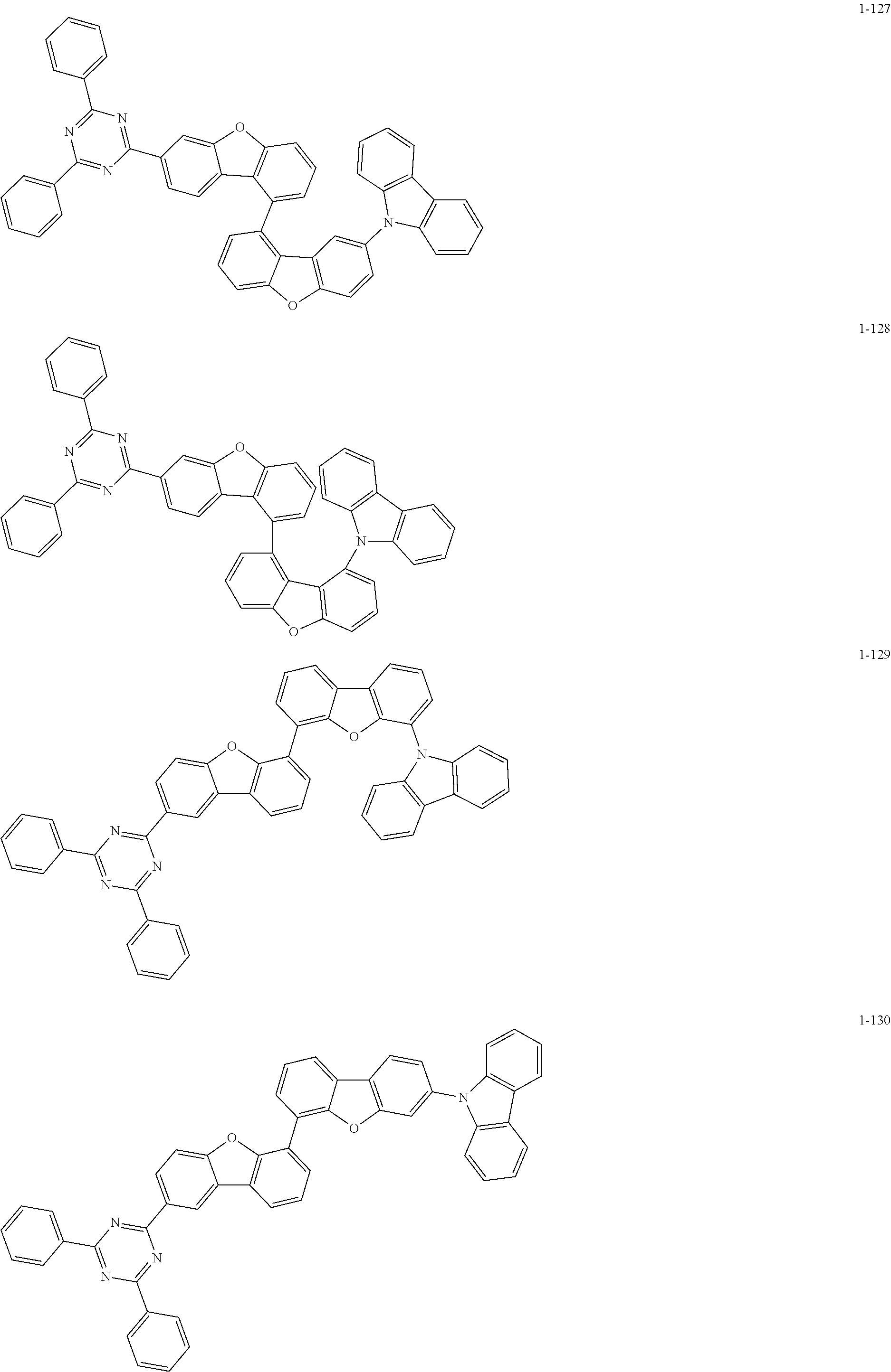

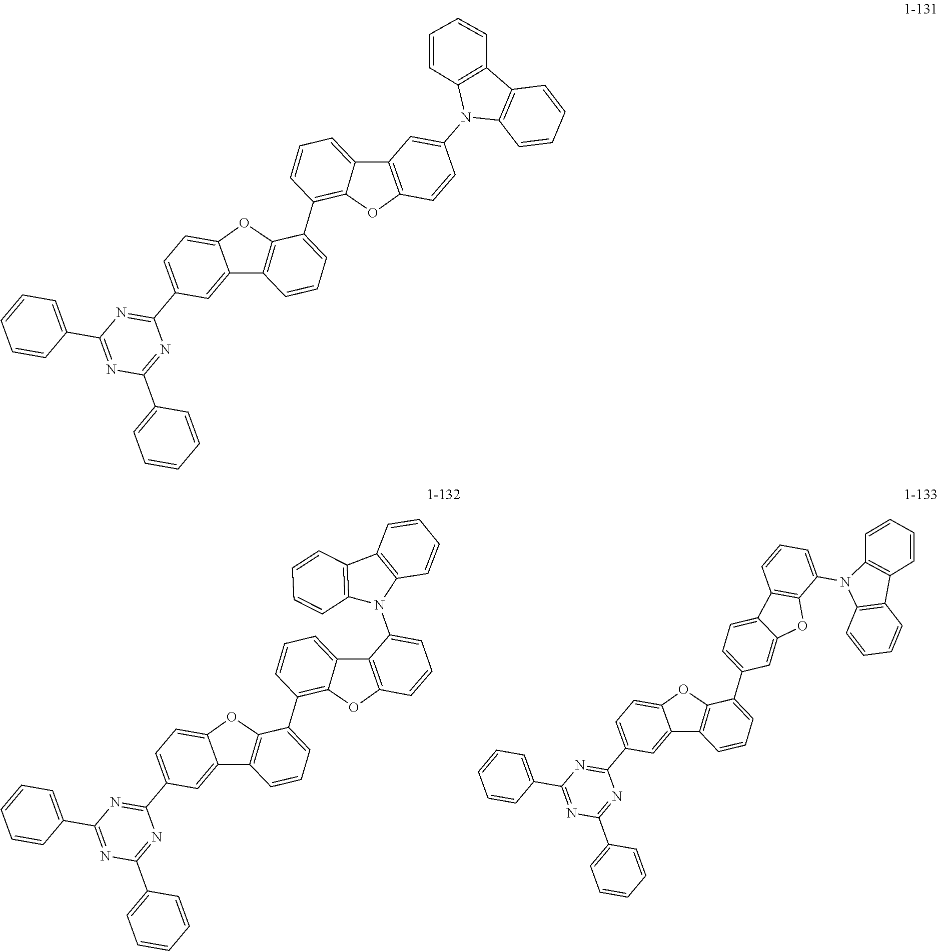 [SMILES: c1ccc(-c2nc(-c3ccccc3)nc(-c3ccc4oc5c(-c6cccc7c6oc6ccc(-n8c9ccccc9c9ccccc98)cc67)cccc5c4c3)n2)cc1 ; c1ccc(-c2nc(-c3ccccc3)nc(-c3ccc4oc5c(-c6cccc7c6oc6cccc(-n8c9ccccc9c9ccccc98)c67)cccc5c4c3)n2)cc1 ; c1ccc(-c2nc(-c3ccccc3)nc(-c3ccc4oc5c(-c6ccc7c(c6)oc6c(-n8c9ccccc9c9ccccc98)cccc67)cccc5c4c3)n2)cc1]

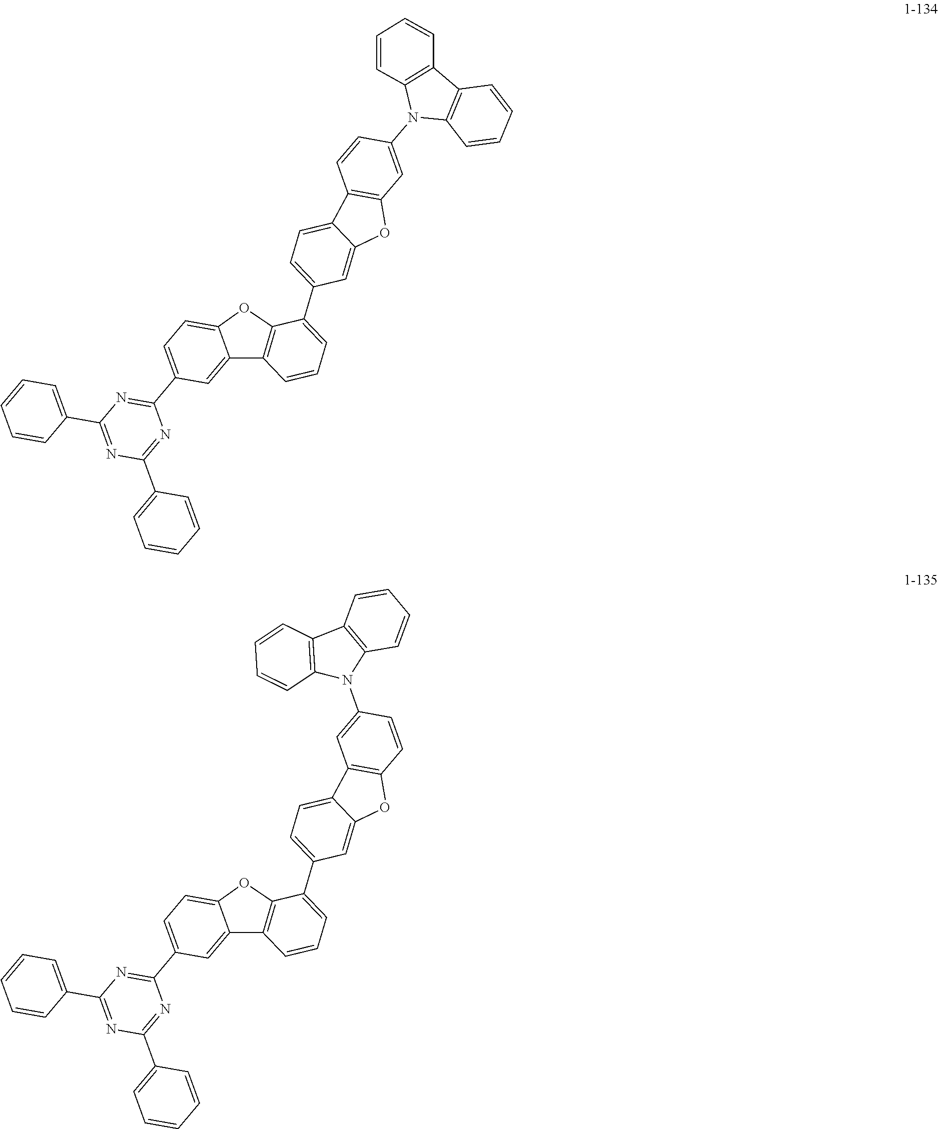 [SMILES: c1ccc(-c2nc(-c3ccccc3)nc(-c3ccc4oc5c(-c6ccc7c(c6)oc6cc(-n8c9ccccc9c9ccccc98)ccc67)cccc5c4c3)n2)cc1 ; c1ccc(-c2nc(-c3ccccc3)nc(-c3ccc4oc5c(-c6ccc7c(c6)oc6ccc(-n8c9ccccc9c9ccccc98)cc67)cccc5c4c3)n2)cc1]

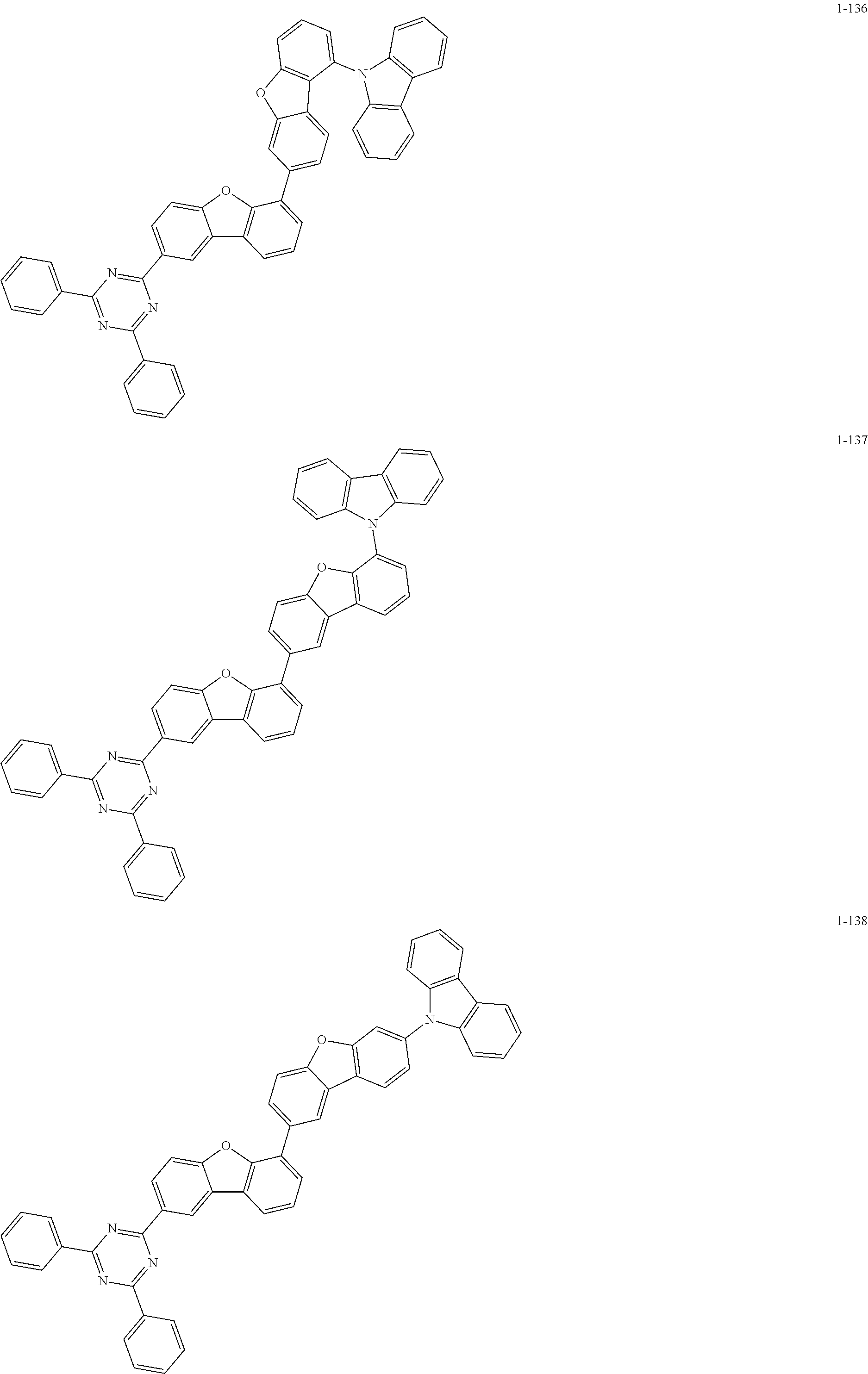 [SMILES: c1ccc(-c2nc(-c3ccccc3)nc(-c3ccc4oc5c(-c6ccc7c(c6)oc6cccc(-n8c9ccccc9c9ccccc98)c67)cccc5c4c3)n2)cc1 ; c1ccc(-c2nc(-c3ccccc3)nc(-c3ccc4oc5c(-c6ccc7oc8c(-n9c%10ccccc%10c%10ccccc%109)cccc8c7c6)cccc5c4c3)n2)cc1 ; c1ccc(-c2nc(-c3ccccc3)nc(-c3ccc4oc5c(-c6ccc7oc8cc(-n9c%10ccccc%10c%10ccccc%109)ccc8c7c6)cccc5c4c3)n2)cc1]

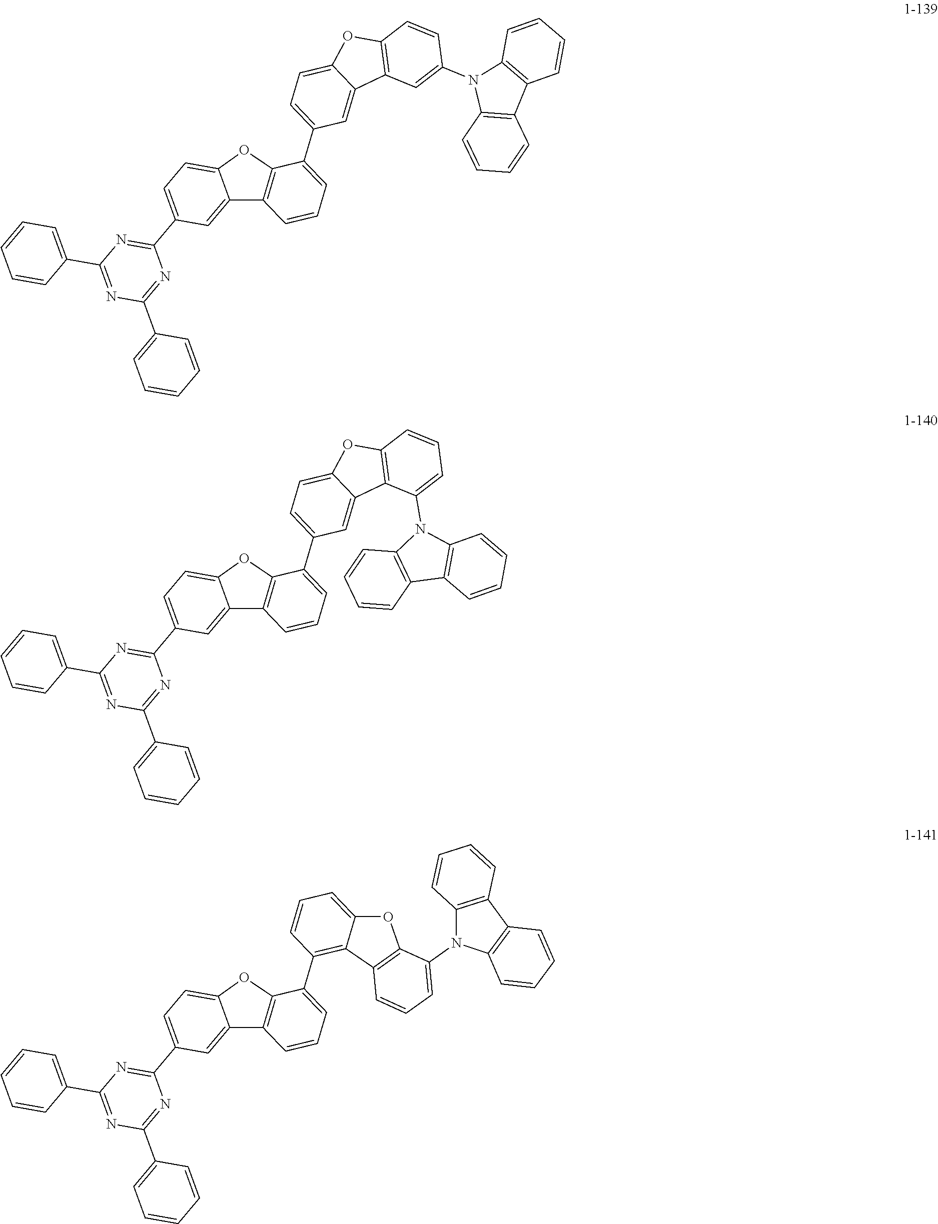 [SMILES: c1ccc(-c2nc(-c3ccccc3)nc(-c3ccc4oc5c(-c6ccc7oc8ccc(-n9c%10ccccc%10c%10ccccc%109)cc8c7c6)cccc5c4c3)n2)cc1 ; c1ccc(-c2nc(-c3ccccc3)nc(-c3ccc4oc5c(-c6ccc7oc8cccc(-n9c%10ccccc%10c%10ccccc%109)c8c7c6)cccc5c4c3)n2)cc1 ; c1ccc(-c2nc(-c3ccccc3)nc(-c3ccc4oc5c(-c6cccc7oc8c(-n9c%10ccccc%10c%10ccccc%109)cccc8c67)cccc5c4c3)n2)cc1]

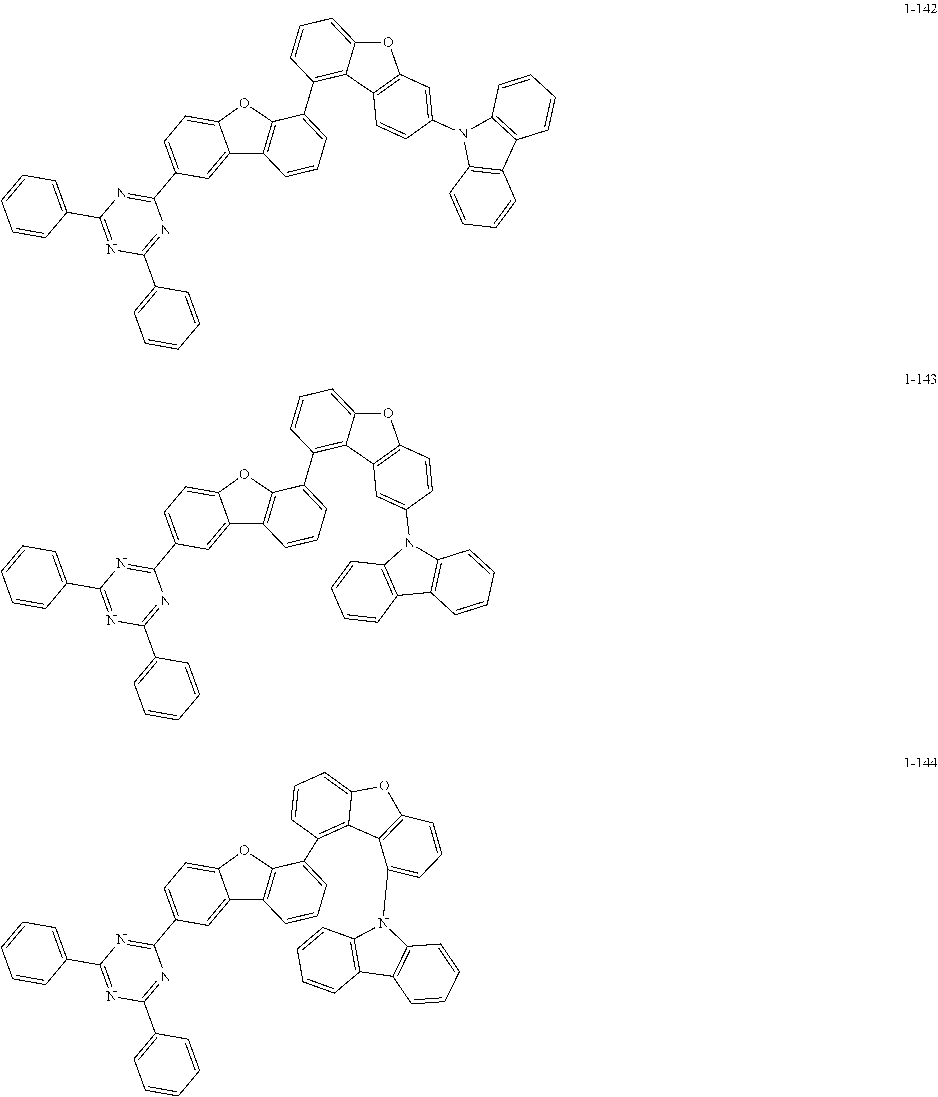 [SMILES: c1ccc(-c2nc(-c3ccccc3)nc(-c3ccc4oc5c(-c6cccc7oc8cc(-n9c%10ccccc%10c%10ccccc%109)ccc8c67)cccc5c4c3)n2)cc1 ; c1ccc(-c2nc(-c3ccccc3)nc(-c3ccc4oc5c(-c6cccc7oc8ccc(-n9c%10ccccc%10c%10ccccc%109)cc8c67)cccc5c4c3)n2)cc1 ; c1ccc(-c2nc(-c3ccccc3)nc(-c3ccc4oc5c(-c6cccc7oc8cccc(-n9c%10ccccc%10c%10ccccc%109)c8c67)cccc5c4c3)n2)cc1]

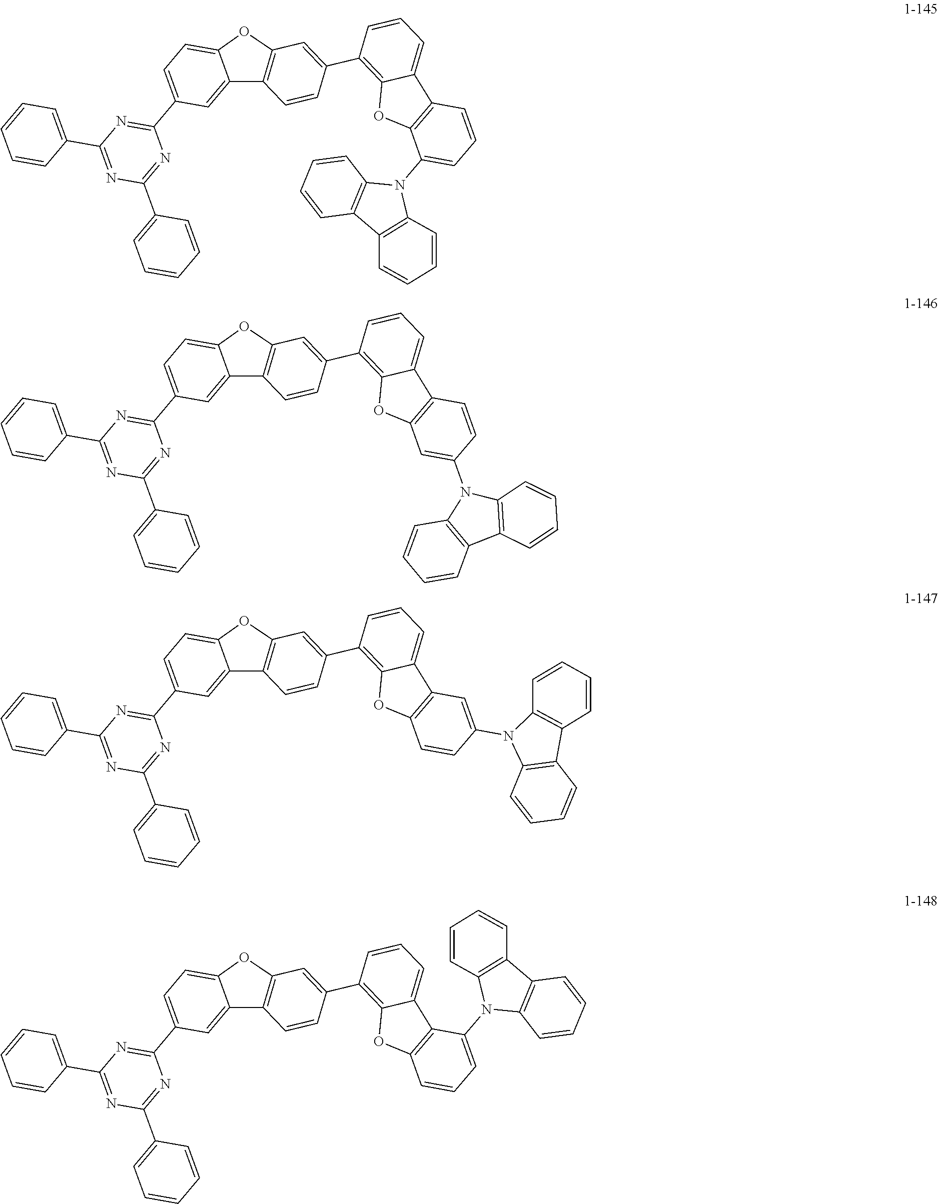

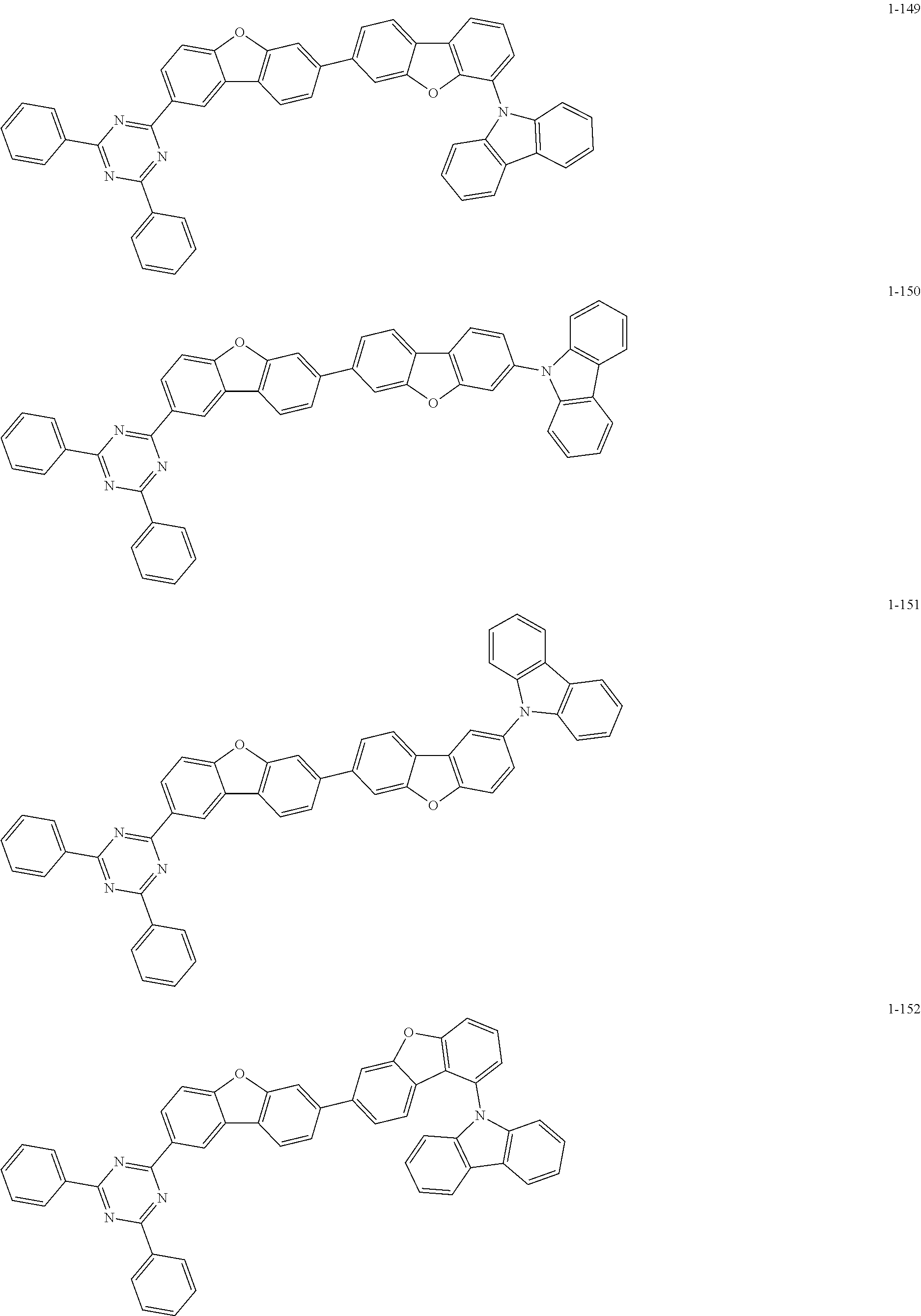 [SMILES: c1ccc(-c2nc(-c3ccccc3)nc(-c3ccc4oc5cc(-c6ccc7c(c6)oc6c(-n8c9ccccc9c9ccccc98)cccc67)ccc5c4c3)n2)cc1 ; c1ccc(-c2nc(-c3ccccc3)nc(-c3ccc4oc5cc(-c6ccc7c(c6)oc6cc(-n8c9ccccc9c9ccccc98)ccc67)ccc5c4c3)n2)cc1 ; c1ccc(-c2nc(-c3ccccc3)nc(-c3ccc4oc5cc(-c6ccc7c(c6)oc6ccc(-n8c9ccccc9c9ccccc98)cc67)ccc5c4c3)n2)cc1 ; c1ccc(-c2nc(-c3ccccc3)nc(-c3ccc4oc5cc(-c6ccc7c(c6)oc6cccc(-n8c9ccccc9c9ccccc98)c67)ccc5c4c3)n2)cc1]

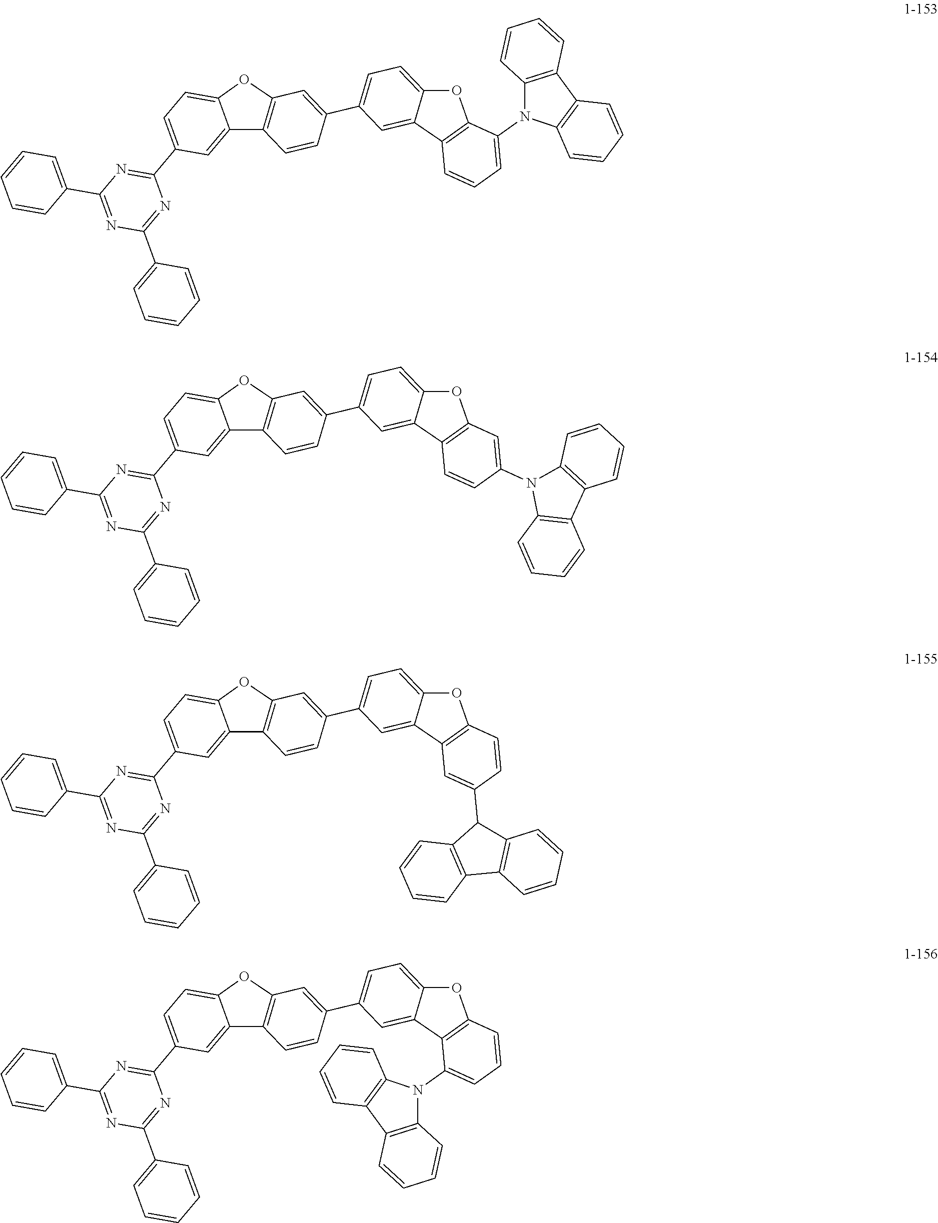 [SMILES: c1ccc(-c2nc(-c3ccccc3)nc(-c3ccc4oc5cc(-c6ccc7oc8c(-n9c%10ccccc%10c%10ccccc%109)cccc8c7c6)ccc5c4c3)n2)cc1 ; c1ccc(-c2nc(-c3ccccc3)nc(-c3ccc4oc5cc(-c6ccc7oc8cc(-n9c%10ccccc%10c%10ccccc%109)ccc8c7c6)ccc5c4c3)n2)cc1 ; c1ccc(-c2nc(-c3ccccc3)nc(-c3ccc4oc5cc(-c6ccc7oc8ccc(C9c%10ccccc%10-c%10ccccc%109)cc8c7c6)ccc5c4c3)n2)cc1 ; c1ccc(-c2nc(-c3ccccc3)nc(-c3ccc4oc5cc(-c6ccc7oc8cccc(-n9c%10ccccc%10c%10ccccc%109)c8c7c6)ccc5c4c3)n2)cc1]

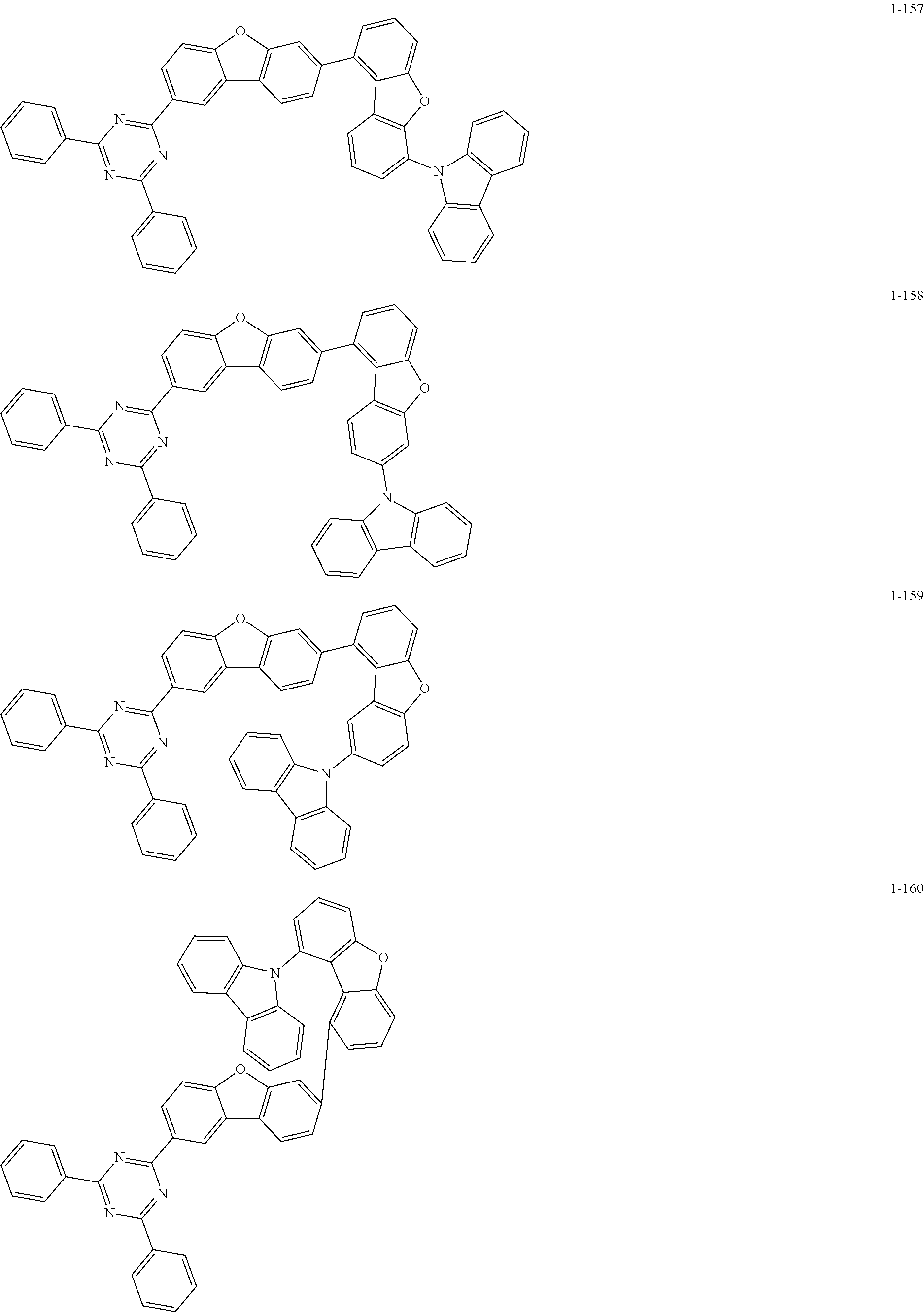

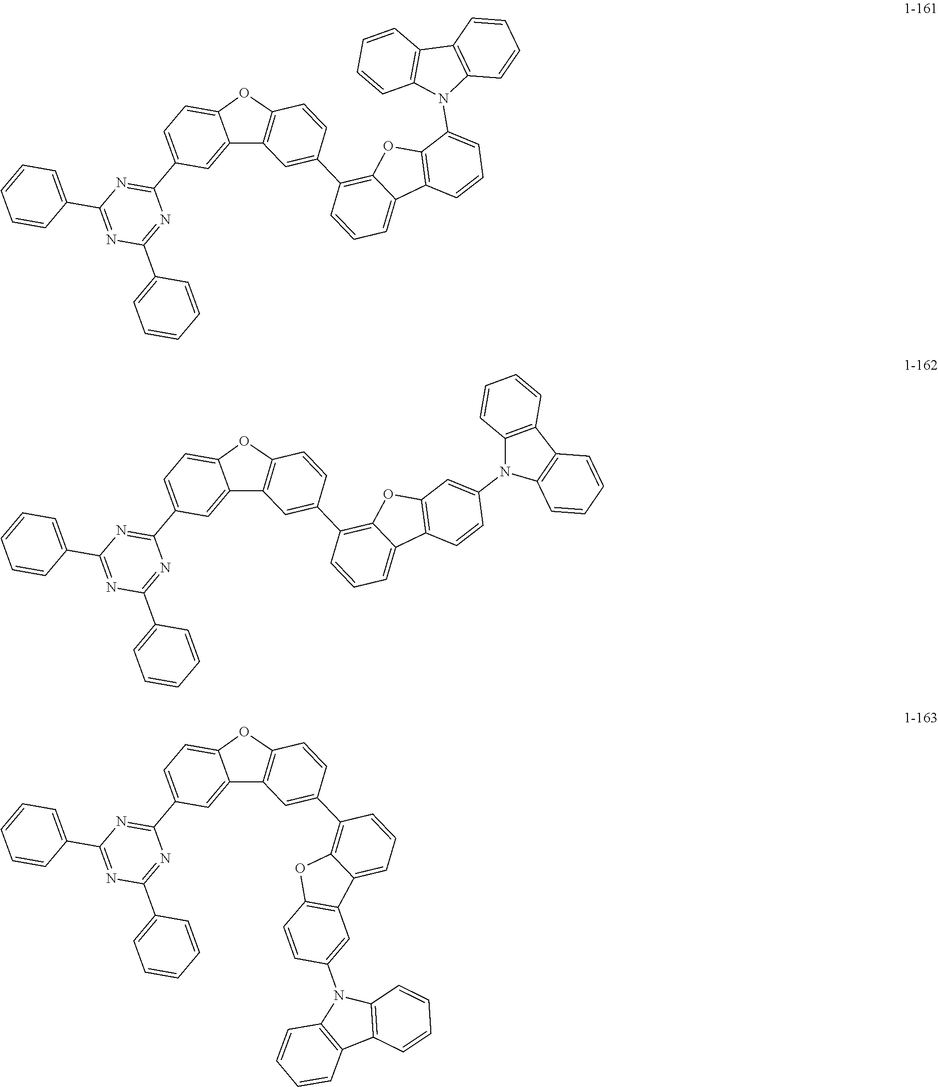 [SMILES: c1ccc(-c2nc(-c3ccccc3)nc(-c3ccc4oc5ccc(-c6cccc7c6oc6c(-n8c9ccccc9c9ccccc98)cccc67)cc5c4c3)n2)cc1 ; c1ccc(-c2nc(-c3ccccc3)nc(-c3ccc4oc5ccc(-c6cccc7c6oc6cc(-n8c9ccccc9c9ccccc98)ccc67)cc5c4c3)n2)cc1 ; c1ccc(-c2nc(-c3ccccc3)nc(-c3ccc4oc5ccc(-c6cccc7c6oc6ccc(-n8c9ccccc9c9ccccc98)cc67)cc5c4c3)n2)cc1]

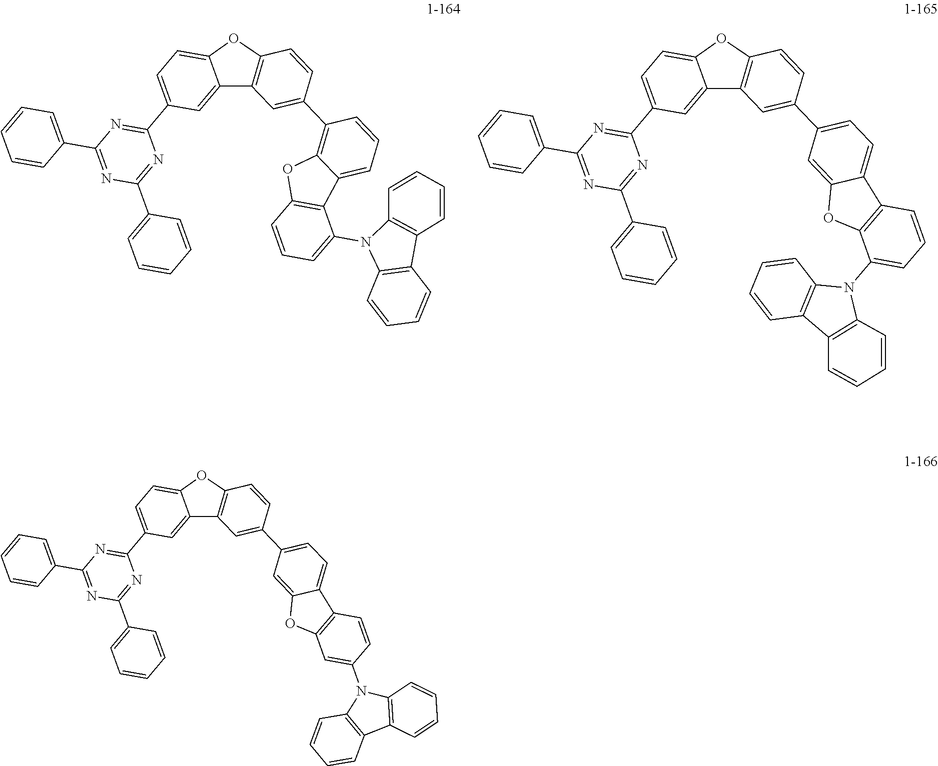 [SMILES: c1ccc(-c2nc(-c3ccccc3)nc(-c3ccc4oc5ccc(-c6cccc7c6oc6cccc(-n8c9ccccc9c9ccccc98)c67)cc5c4c3)n2)cc1 ; c1ccc(-c2nc(-c3ccccc3)nc(-c3ccc4oc5ccc(-c6ccc7c(c6)oc6c(-n8c9ccccc9c9ccccc98)cccc67)cc5c4c3)n2)cc1 ; c1ccc(-c2nc(-c3ccccc3)nc(-c3ccc4oc5ccc(-c6ccc7c(c6)oc6cc(-n8c9ccccc9c9ccccc98)ccc67)cc5c4c3)n2)cc1]

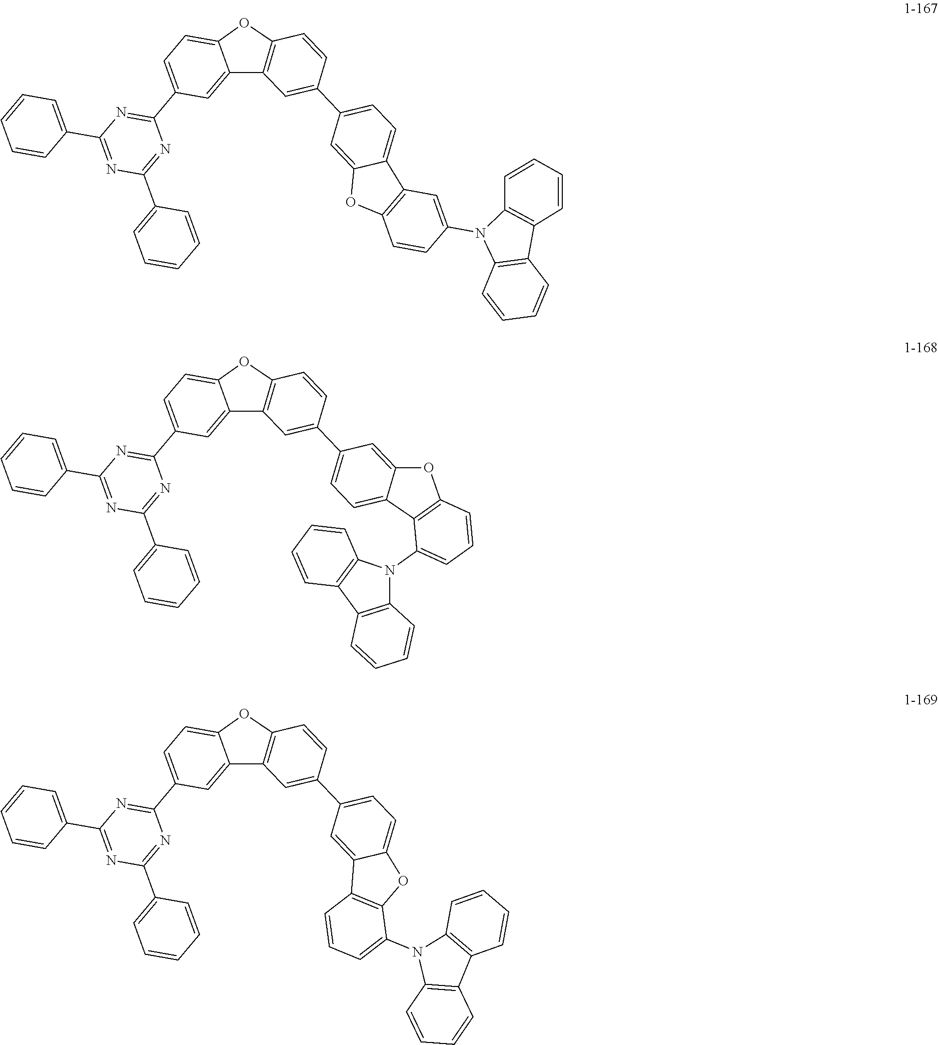 [SMILES: c1ccc(-c2nc(-c3ccccc3)nc(-c3ccc4oc5ccc(-c6ccc7c(c6)oc6ccc(-n8c9ccccc9c9ccccc98)cc67)cc5c4c3)n2)cc1 ; c1ccc(-c2nc(-c3ccccc3)nc(-c3ccc4oc5ccc(-c6ccc7c(c6)oc6cccc(-n8c9ccccc9c9ccccc98)c67)cc5c4c3)n2)cc1 ; c1ccc(-c2nc(-c3ccccc3)nc(-c3ccc4oc5ccc(-c6ccc7oc8c(-n9c%10ccccc%10c%10ccccc%109)cccc8c7c6)cc5c4c3)n2)cc1]

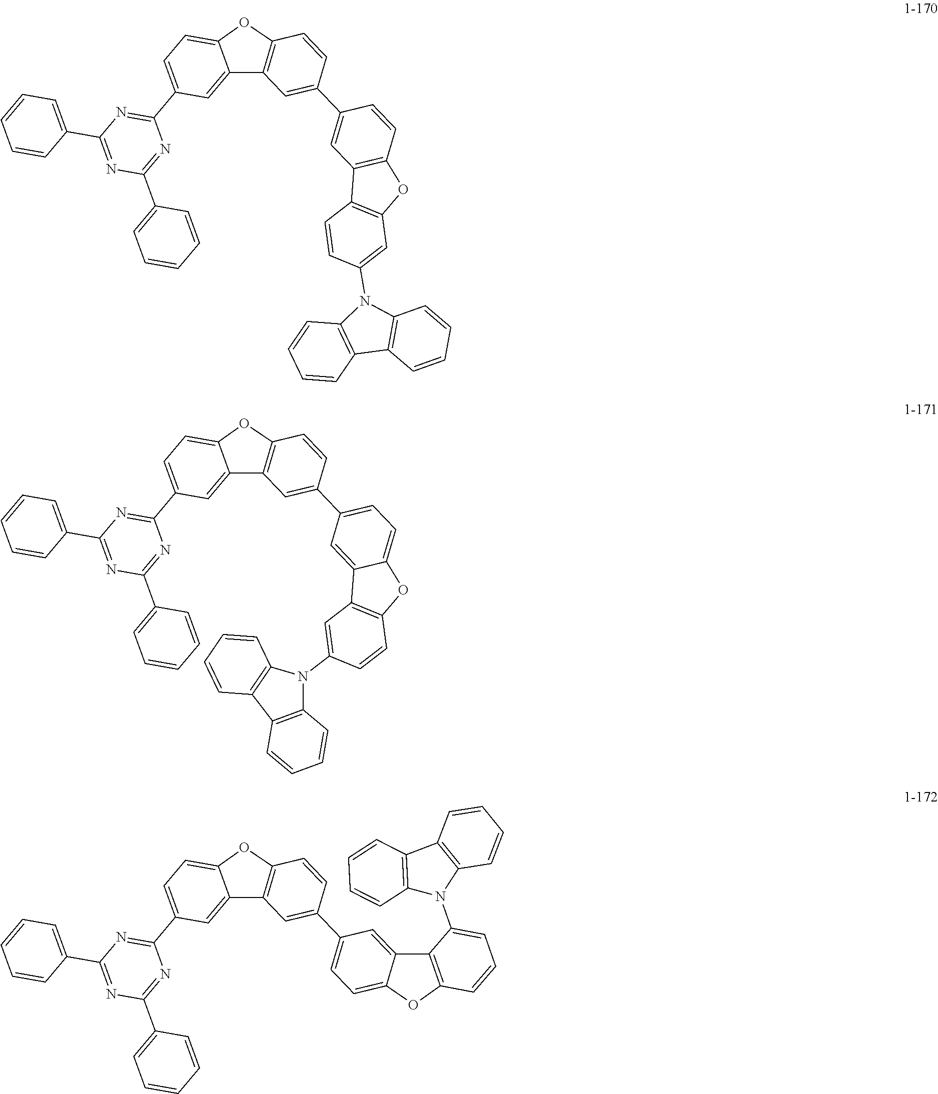 [SMILES: c1ccc(-c2nc(-c3ccccc3)nc(-c3ccc4oc5ccc(-c6ccc7oc8cc(-n9c%10ccccc%10c%10ccccc%109)ccc8c7c6)cc5c4c3)n2)cc1 ; c1ccc(-c2nc(-c3ccccc3)nc(-c3ccc4oc5ccc(-c6ccc7oc8ccc(-n9c%10ccccc%10c%10ccccc%109)cc8c7c6)cc5c4c3)n2)cc1 ; c1ccc(-c2nc(-c3ccccc3)nc(-c3ccc4oc5ccc(-c6ccc7oc8cccc(-n9c%10ccccc%10c%10ccccc%109)c8c7c6)cc5c4c3)n2)cc1]

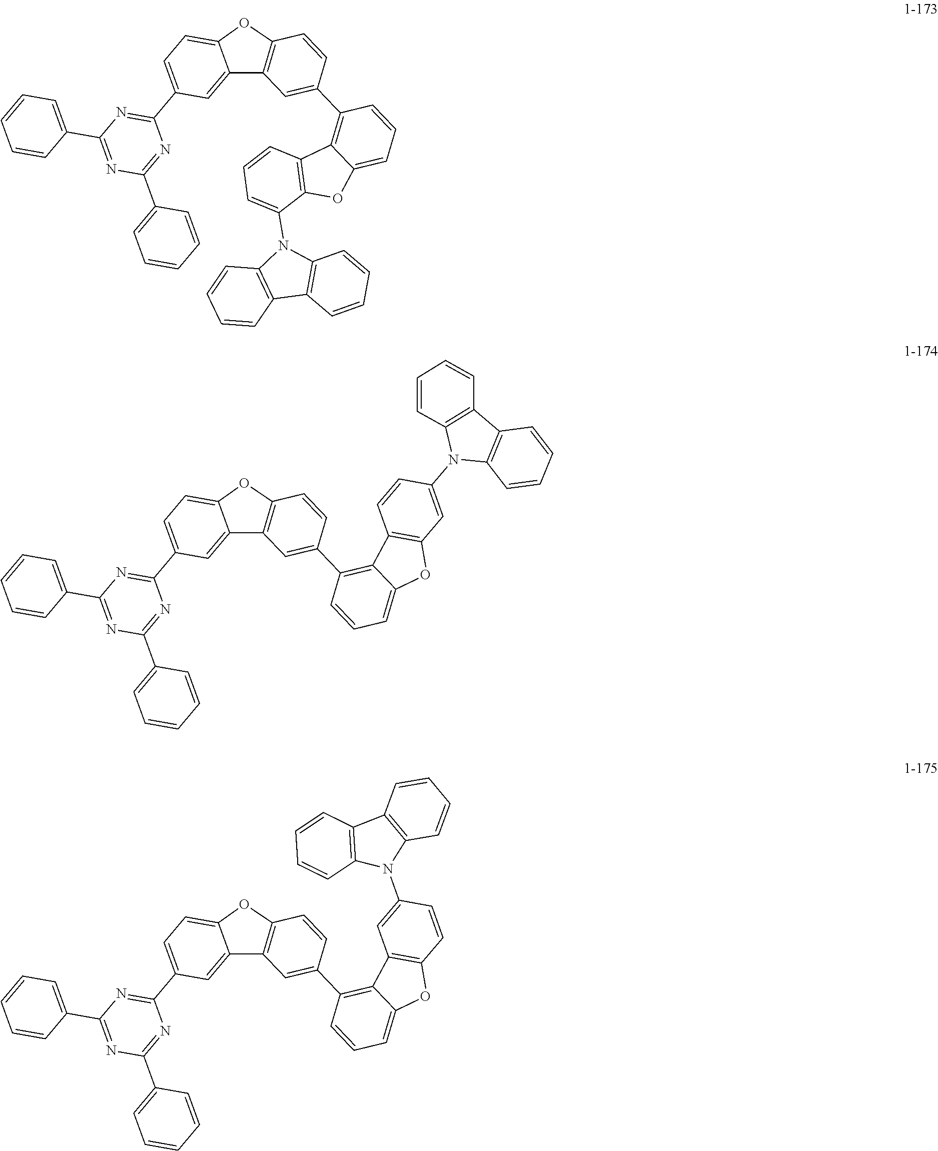 [SMILES: c1ccc(-c2nc(-c3ccccc3)nc(-c3ccc4oc5ccc(-c6cccc7oc8c(-n9c%10ccccc%10c%10ccccc%109)cccc8c67)cc5c4c3)n2)cc1 ; c1ccc(-c2nc(-c3ccccc3)nc(-c3ccc4oc5ccc(-c6cccc7oc8cc(-n9c%10ccccc%10c%10ccccc%109)ccc8c67)cc5c4c3)n2)cc1 ; c1ccc(-c2nc(-c3ccccc3)nc(-c3ccc4oc5ccc(-c6cccc7oc8ccc(-n9c%10ccccc%10c%10ccccc%109)cc8c67)cc5c4c3)n2)cc1]

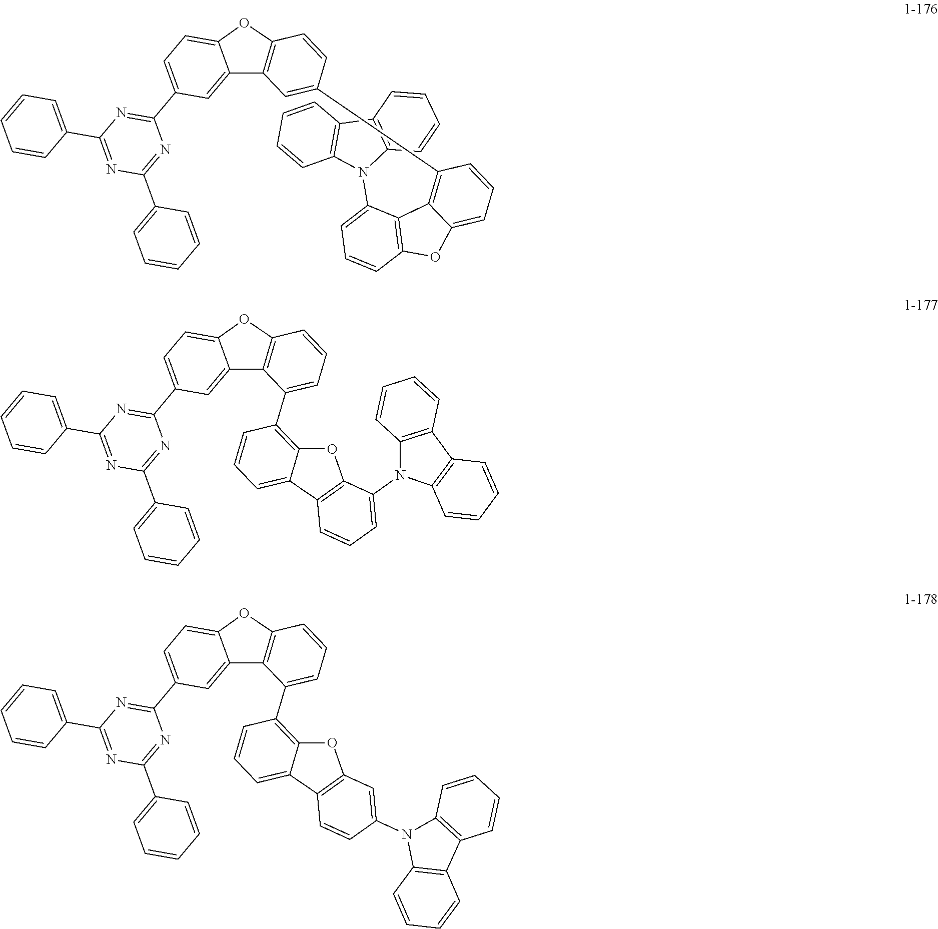 [SMILES: c1ccc(-c2nc(-c3ccccc3)nc(-c3ccc4oc5ccc(-c6cccc7oc8cccc(-n9c%10ccccc%10c%10ccccc%109)c8c67)cc5c4c3)n2)cc1 ; c1ccc(-c2nc(-c3ccccc3)nc(-c3ccc4oc5cccc(-c6cccc7c6oc6c(-n8c9ccccc9c9ccccc98)cccc67)c5c4c3)n2)cc1 ; c1ccc(-c2nc(-c3ccccc3)nc(-c3ccc4oc5cccc(-c6cccc7c6oc6cc(-n8c9ccccc9c9ccccc98)ccc67)c5c4c3)n2)cc1]

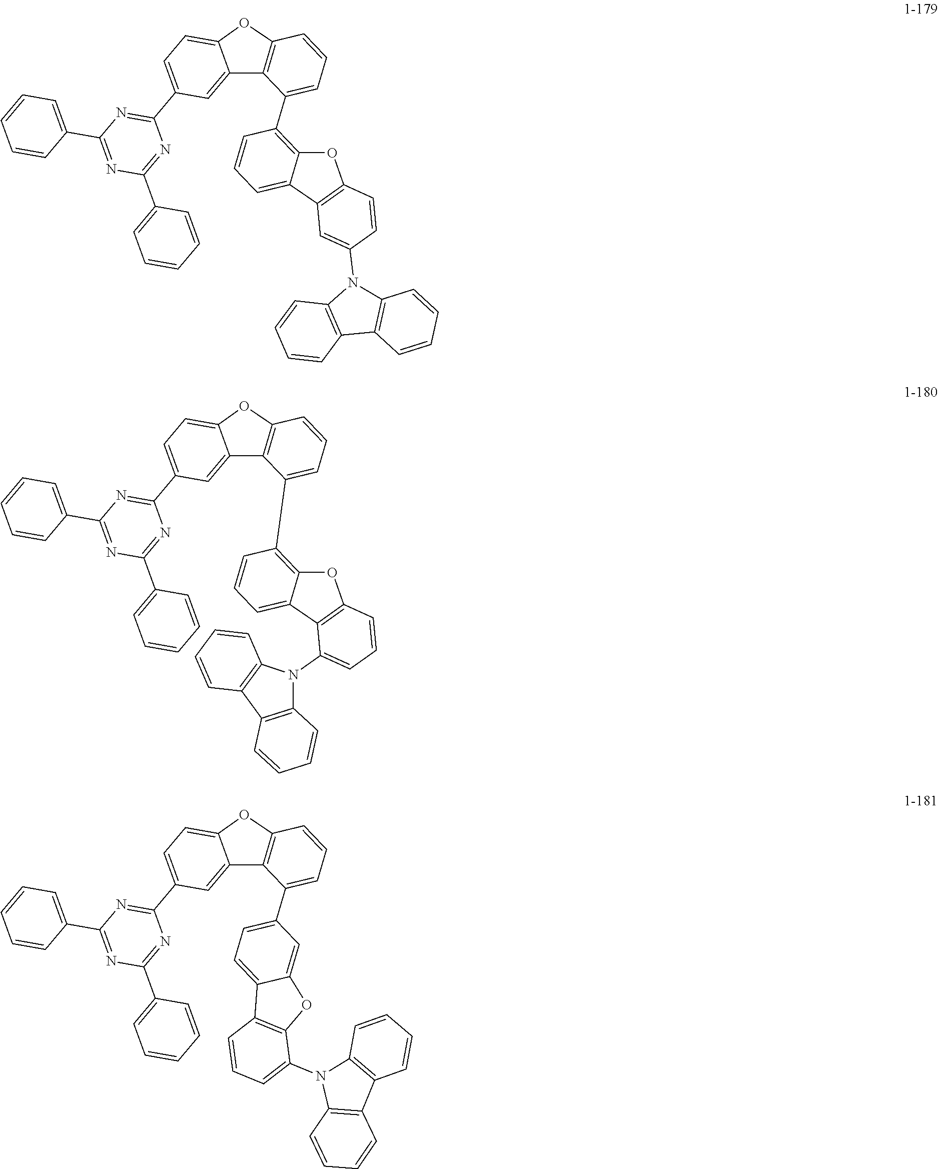 [SMILES: c1ccc(-c2nc(-c3ccccc3)nc(-c3ccc4oc5cccc(-c6cccc7c6oc6ccc(-n8c9ccccc9c9ccccc98)cc67)c5c4c3)n2)cc1 ; c1ccc(-c2nc(-c3ccccc3)nc(-c3ccc4oc5cccc(-c6cccc7c6oc6cccc(-n8c9ccccc9c9ccccc98)c67)c5c4c3)n2)cc1 ; c1ccc(-c2nc(-c3ccccc3)nc(-c3ccc4oc5cccc(-c6ccc7c(c6)oc6c(-n8c9ccccc9c9ccccc98)cccc67)c5c4c3)n2)cc1]

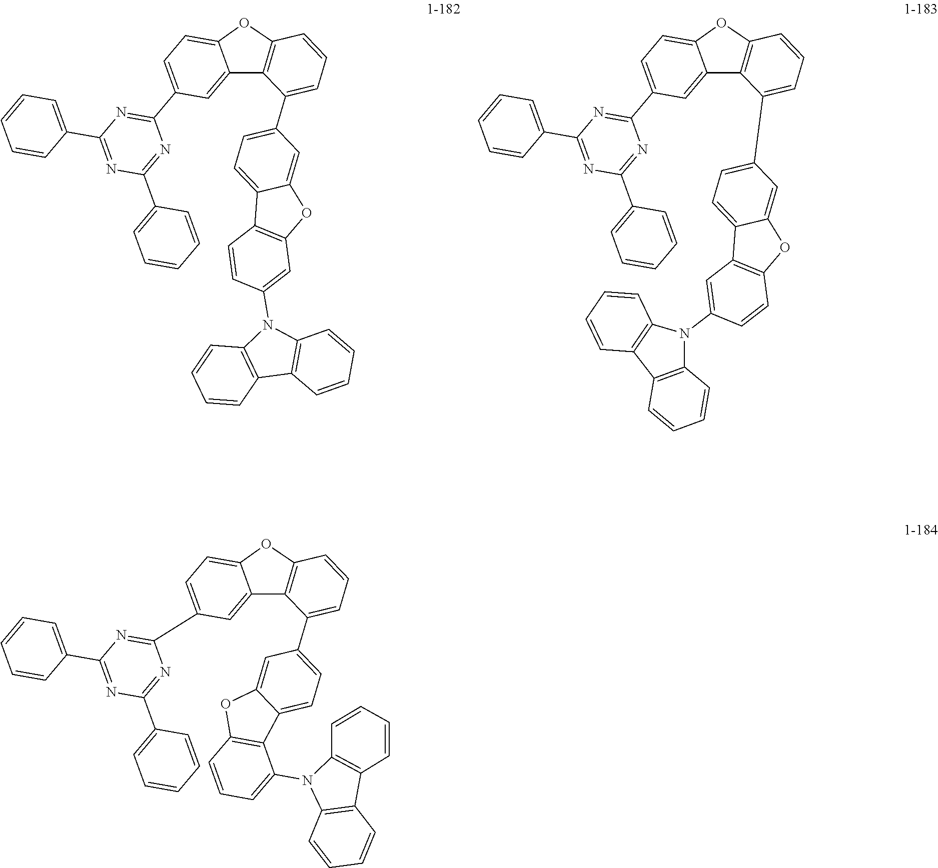 [SMILES: c1ccc(-c2nc(-c3ccccc3)nc(-c3ccc4oc5cccc(-c6ccc7c(c6)oc6cc(-n8c9ccccc9c9ccccc98)ccc67)c5c4c3)n2)cc1 ; c1ccc(-c2nc(-c3ccccc3)nc(-c3ccc4oc5cccc(-c6ccc7c(c6)oc6ccc(-n8c9ccccc9c9ccccc98)cc67)c5c4c3)n2)cc1 ; c1ccc(-c2nc(-c3ccccc3)nc(-c3ccc4oc5cccc(-c6ccc7c(c6)oc6cccc(-n8c9ccccc9c9ccccc98)c67)c5c4c3)n2)cc1]

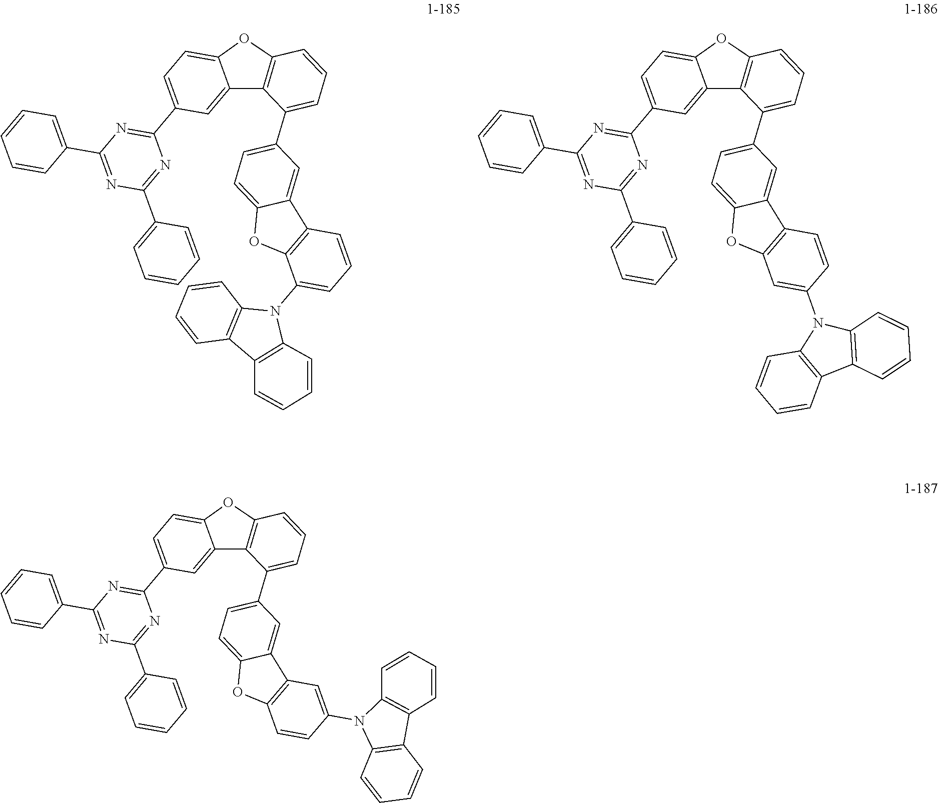 [SMILES: c1ccc(-c2nc(-c3ccccc3)nc(-c3ccc4oc5cccc(-c6ccc7oc8c(-n9c%10ccccc%10c%10ccccc%109)cccc8c7c6)c5c4c3)n2)cc1 ; c1ccc(-c2nc(-c3ccccc3)nc(-c3ccc4oc5cccc(-c6ccc7oc8cc(-n9c%10ccccc%10c%10ccccc%109)ccc8c7c6)c5c4c3)n2)cc1 ; c1ccc(-c2nc(-c3ccccc3)nc(-c3ccc4oc5cccc(-c6ccc7oc8ccc(-n9c%10ccccc%10c%10ccccc%109)cc8c7c6)c5c4c3)n2)cc1]

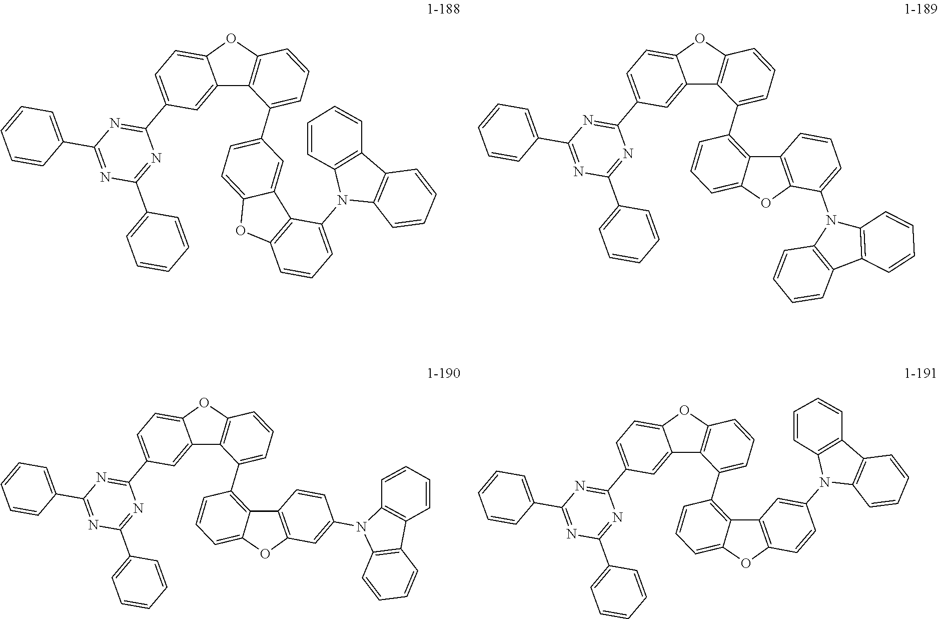 [SMILES: c1ccc(-c2nc(-c3ccccc3)nc(-c3ccc4oc5cccc(-c6ccc7oc8cccc(-n9c%10ccccc%10c%10ccccc%109)c8c7c6)c5c4c3)n2)cc1 ; c1ccc(-c2nc(-c3ccccc3)nc(-c3ccc4oc5cccc(-c6cccc7oc8c(-n9c%10ccccc%10c%10ccccc%109)cccc8c67)c5c4c3)n2)cc1 ; c1ccc(-c2nc(-c3ccccc3)nc(-c3ccc4oc5cccc(-c6cccc7oc8cc(-n9c%10ccccc%10c%10ccccc%109)ccc8c67)c5c4c3)n2)cc1 ; c1ccc(-c2nc(-c3ccccc3)nc(-c3ccc4oc5cccc(-c6cccc7oc8ccc(-n9c%10ccccc%10c%10ccccc%109)cc8c67)c5c4c3)n2)cc1]

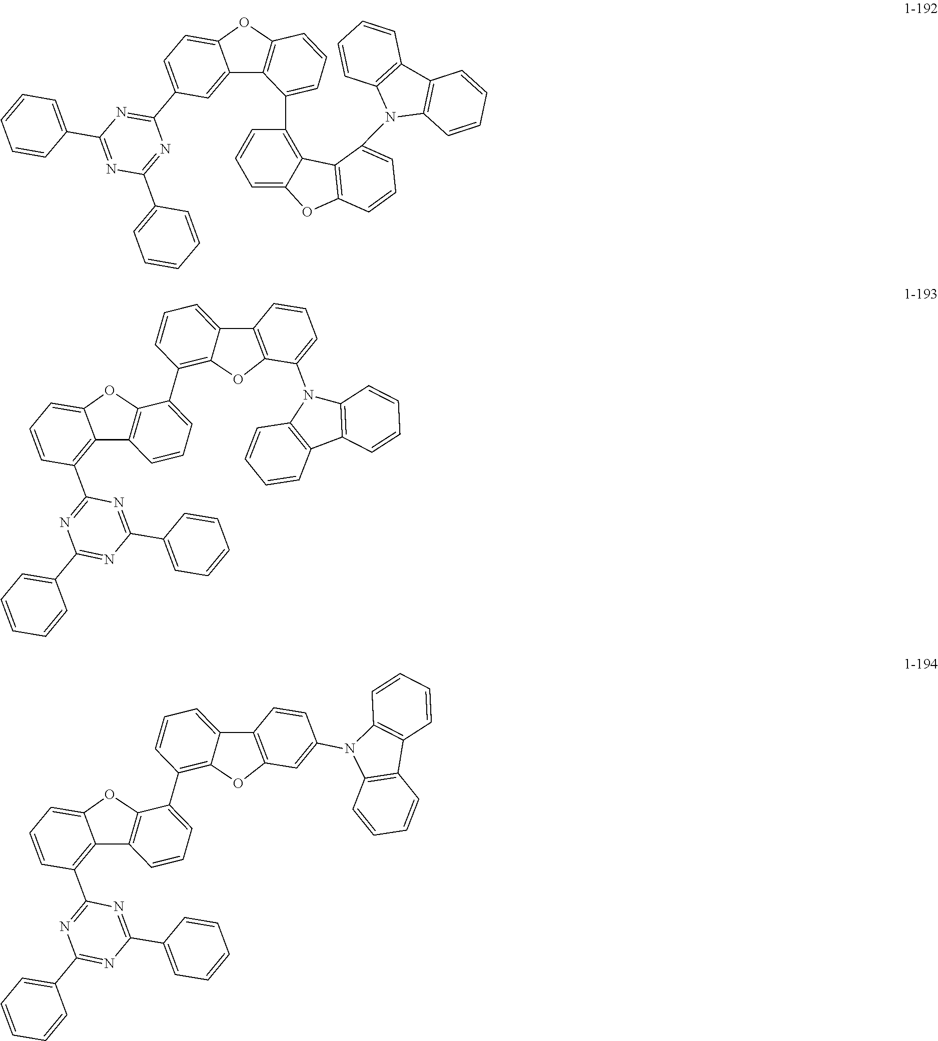

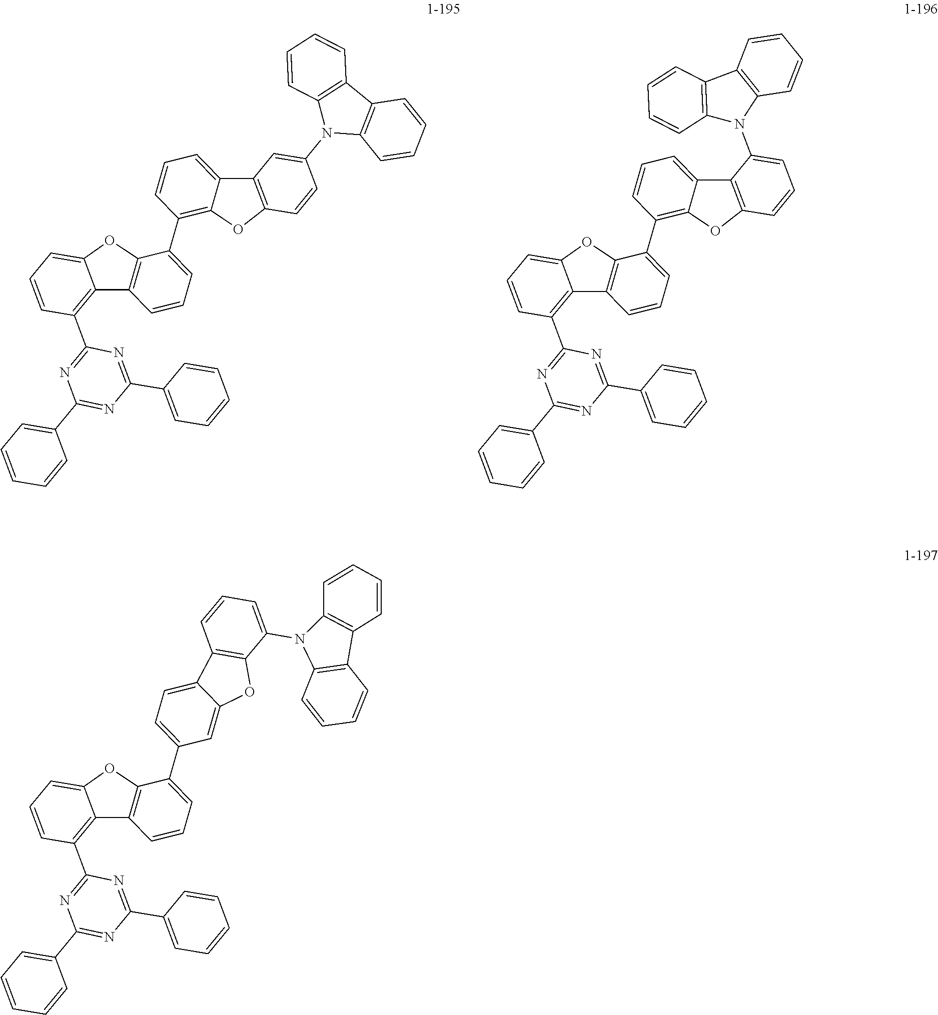 [SMILES: c1ccc(-c2nc(-c3ccccc3)nc(-c3cccc4oc5c(-c6cccc7c6oc6ccc(-n8c9ccccc9c9ccccc98)cc67)cccc5c34)n2)cc1 ; c1ccc(-c2nc(-c3ccccc3)nc(-c3cccc4oc5c(-c6cccc7c6oc6cccc(-n8c9ccccc9c9ccccc98)c67)cccc5c34)n2)cc1 ; c1ccc(-c2nc(-c3ccccc3)nc(-c3cccc4oc5c(-c6ccc7c(c6)oc6c(-n8c9ccccc9c9ccccc98)cccc67)cccc5c34)n2)cc1]

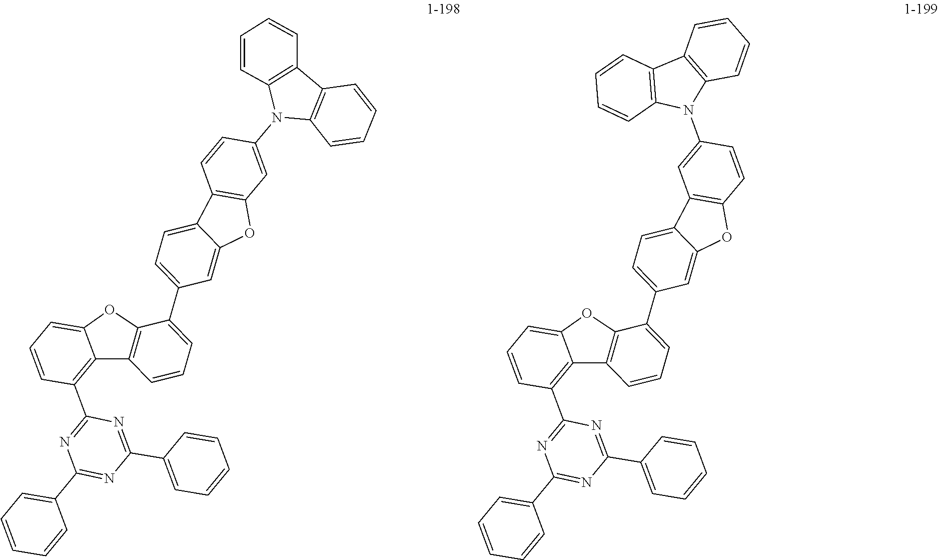 [SMILES: c1ccc(-c2nc(-c3ccccc3)nc(-c3cccc4oc5c(-c6ccc7c(c6)oc6cc(-n8c9ccccc9c9ccccc98)ccc67)cccc5c34)n2)cc1 ; c1ccc(-c2nc(-c3ccccc3)nc(-c3cccc4oc5c(-c6ccc7c(c6)oc6ccc(-n8c9ccccc9c9ccccc98)cc67)cccc5c34)n2)cc1]

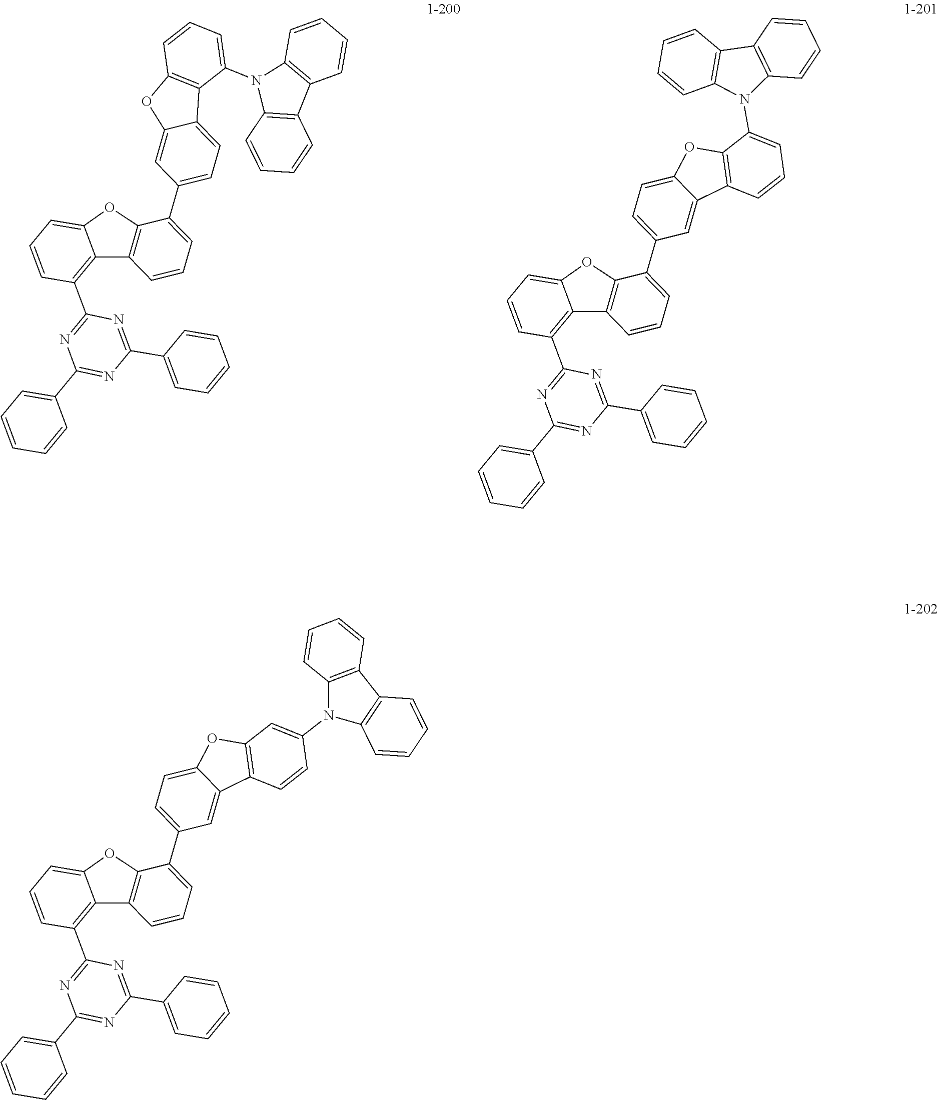 [SMILES: c1ccc(-c2nc(-c3ccccc3)nc(-c3cccc4oc5c(-c6ccc7c(c6)oc6cccc(-n8c9ccccc9c9ccccc98)c67)cccc5c34)n2)cc1 ; c1ccc(-c2nc(-c3ccccc3)nc(-c3cccc4oc5c(-c6ccc7oc8c(-n9c%10ccccc%10c%10ccccc%109)cccc8c7c6)cccc5c34)n2)cc1 ; c1ccc(-c2nc(-c3ccccc3)nc(-c3cccc4oc5c(-c6ccc7oc8cc(-n9c%10ccccc%10c%10ccccc%109)ccc8c7c6)cccc5c34)n2)cc1]

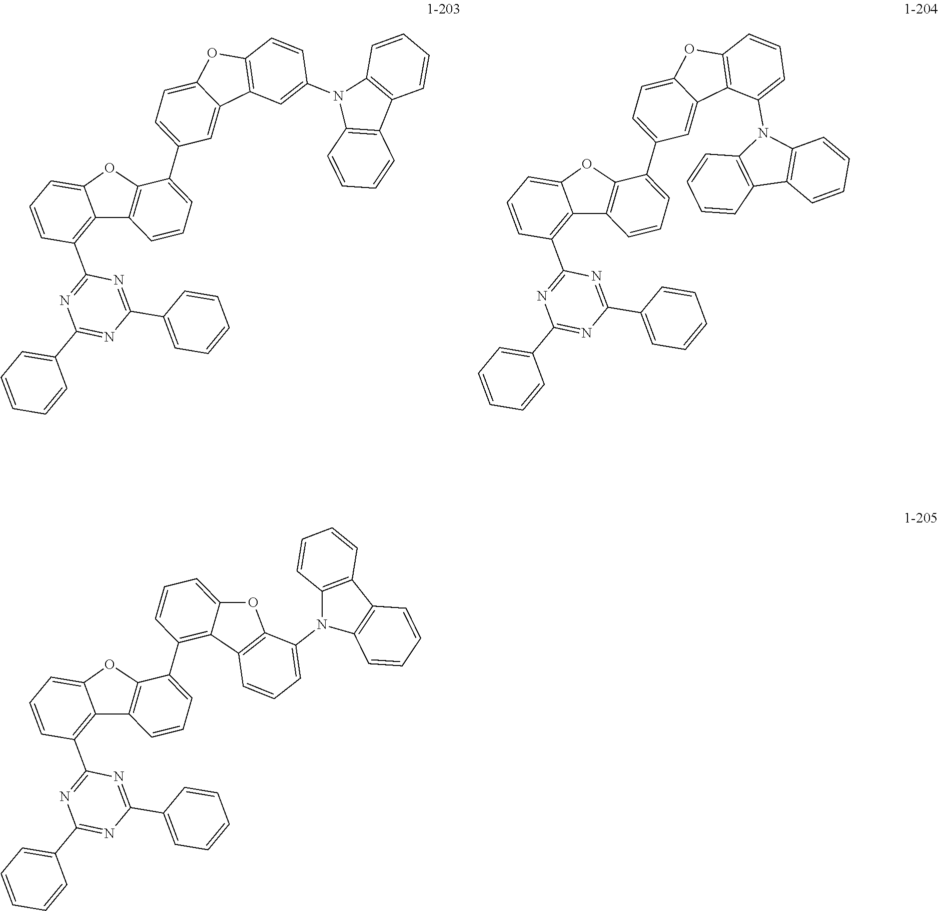 [SMILES: c1ccc(-c2nc(-c3ccccc3)nc(-c3cccc4oc5c(-c6ccc7oc8ccc(-n9c%10ccccc%10c%10ccccc%109)cc8c7c6)cccc5c34)n2)cc1 ; c1ccc(-c2nc(-c3ccccc3)nc(-c3cccc4oc5c(-c6ccc7oc8cccc(-n9c%10ccccc%10c%10ccccc%109)c8c7c6)cccc5c34)n2)cc1 ; c1ccc(-c2nc(-c3ccccc3)nc(-c3cccc4oc5c(-c6cccc7oc8c(-n9c%10ccccc%10c%10ccccc%109)cccc8c67)cccc5c34)n2)cc1]

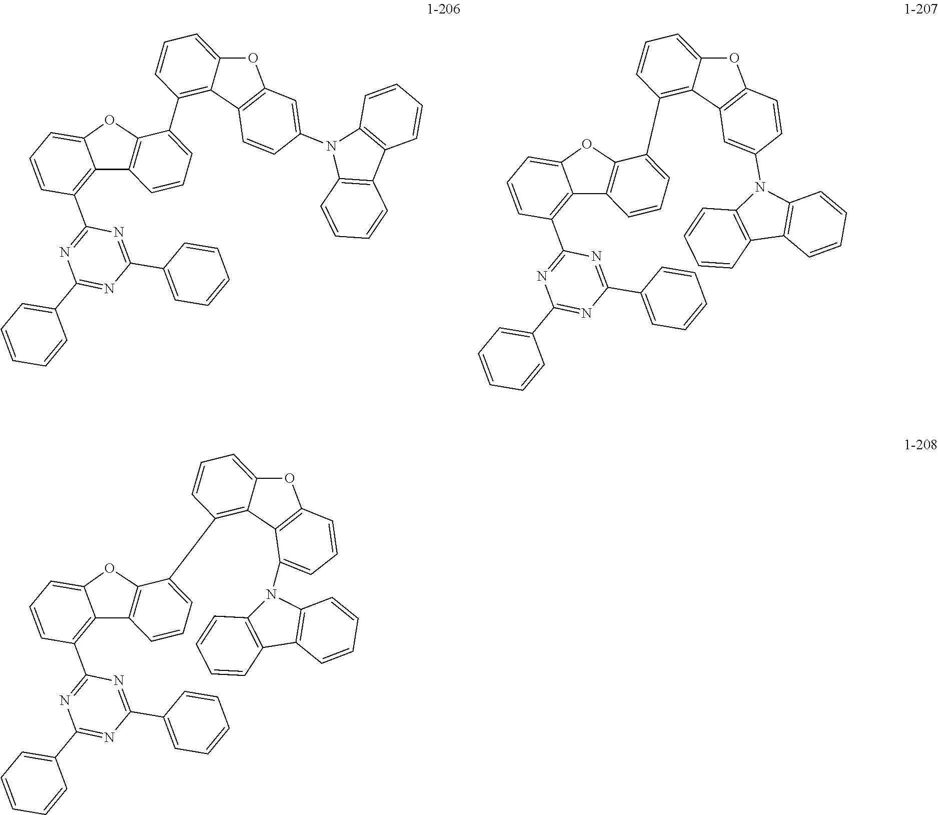 [SMILES: c1ccc(-c2nc(-c3ccccc3)nc(-c3cccc4oc5c(-c6cccc7oc8cc(-n9c%10ccccc%10c%10ccccc%109)ccc8c67)cccc5c34)n2)cc1 ; c1ccc(-c2nc(-c3ccccc3)nc(-c3cccc4oc5c(-c6cccc7oc8ccc(-n9c%10ccccc%10c%10ccccc%109)cc8c67)cccc5c34)n2)cc1 ; c1ccc(-c2nc(-c3ccccc3)nc(-c3cccc4oc5c(-c6cccc7oc8cccc(-n9c%10ccccc%10c%10ccccc%109)c8c67)cccc5c34)n2)cc1]

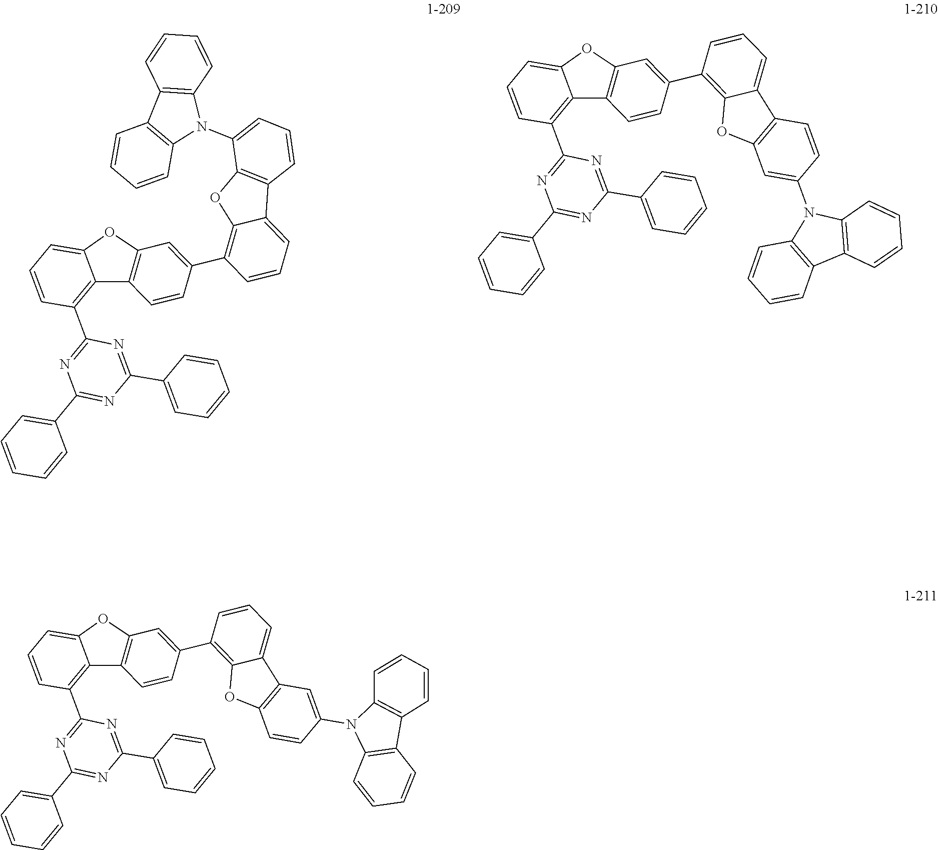 [SMILES: c1ccc(-c2nc(-c3ccccc3)nc(-c3cccc4oc5cc(-c6cccc7c6oc6c(-n8c9ccccc9c9ccccc98)cccc67)ccc5c34)n2)cc1 ; c1ccc(-c2nc(-c3ccccc3)nc(-c3cccc4oc5cc(-c6cccc7c6oc6cc(-n8c9ccccc9c9ccccc98)ccc67)ccc5c34)n2)cc1 ; c1ccc(-c2nc(-c3ccccc3)nc(-c3cccc4oc5cc(-c6cccc7c6oc6ccc(-n8c9ccccc9c9ccccc98)cc67)ccc5c34)n2)cc1]

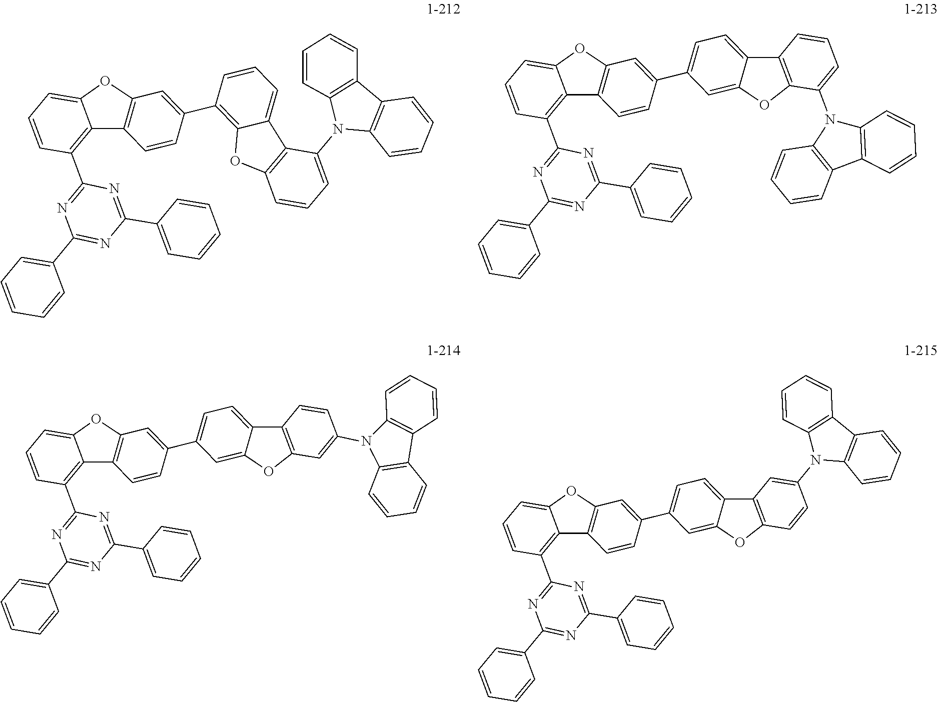 [SMILES: c1ccc(-c2nc(-c3ccccc3)nc(-c3cccc4oc5cc(-c6cccc7c6oc6cccc(-n8c9ccccc9c9ccccc98)c67)ccc5c34)n2)cc1 ; c1ccc(-c2nc(-c3ccccc3)nc(-c3cccc4oc5cc(-c6ccc7c(c6)oc6c(-n8c9ccccc9c9ccccc98)cccc67)ccc5c34)n2)cc1 ; c1ccc(-c2nc(-c3ccccc3)nc(-c3cccc4oc5cc(-c6ccc7c(c6)oc6cc(-n8c9ccccc9c9ccccc98)ccc67)ccc5c34)n2)cc1 ; c1ccc(-c2nc(-c3ccccc3)nc(-c3cccc4oc5cc(-c6ccc7c(c6)oc6ccc(-n8c9ccccc9c9ccccc98)cc67)ccc5c34)n2)cc1]

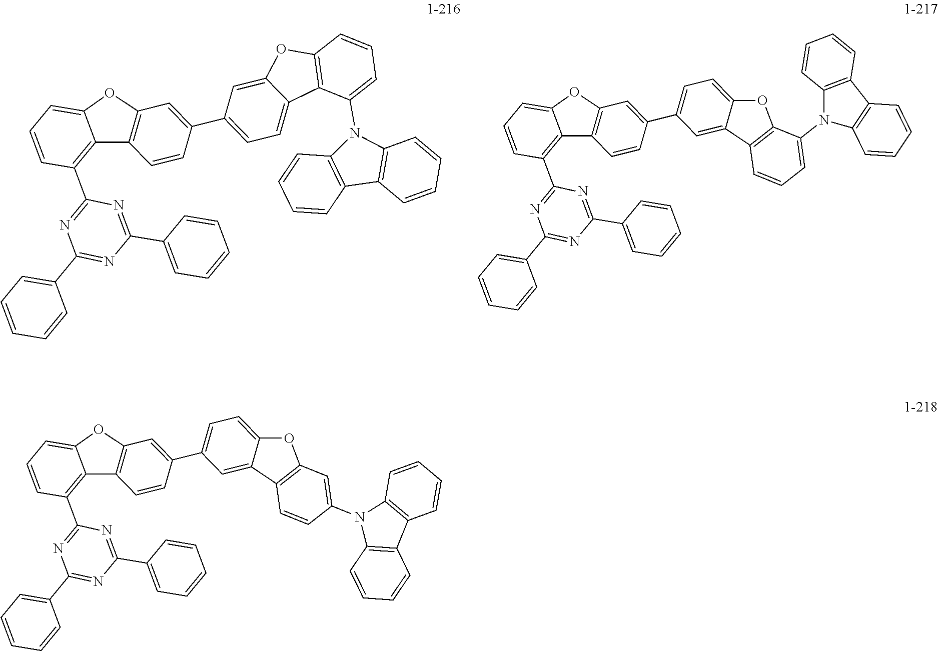 [SMILES: c1ccc(-c2nc(-c3ccccc3)nc(-c3cccc4oc5cc(-c6ccc7c(c6)oc6cccc(-n8c9ccccc9c9ccccc98)c67)ccc5c34)n2)cc1 ; c1ccc(-c2nc(-c3ccccc3)nc(-c3cccc4oc5cc(-c6ccc7oc8c(-n9c%10ccccc%10c%10ccccc%109)cccc8c7c6)ccc5c34)n2)cc1 ; c1ccc(-c2nc(-c3ccccc3)nc(-c3cccc4oc5cc(-c6ccc7oc8cc(-n9c%10ccccc%10c%10ccccc%109)ccc8c7c6)ccc5c34)n2)cc1]

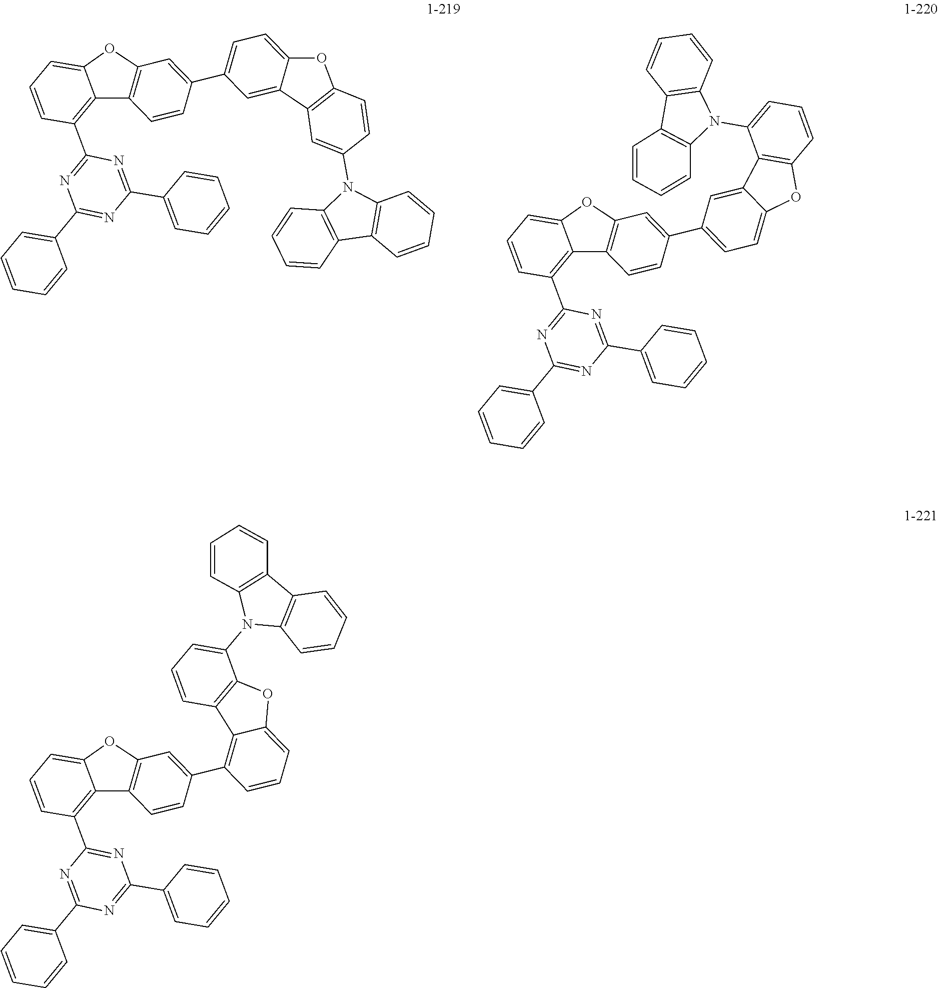 [SMILES: c1ccc(-c2nc(-c3ccccc3)nc(-c3cccc4oc5cc(-c6ccc7oc8ccc(-n9c%10ccccc%10c%10ccccc%109)cc8c7c6)ccc5c34)n2)cc1 ; c1ccc(-c2nc(-c3ccccc3)nc(-c3cccc4oc5cc(-c6ccc7oc8cccc(-n9c%10ccccc%10c%10ccccc%109)c8c7c6)ccc5c34)n2)cc1 ; c1ccc(-c2nc(-c3ccccc3)nc(-c3cccc4oc5cc(-c6cccc7oc8c(-n9c%10ccccc%10c%10ccccc%109)cccc8c67)ccc5c34)n2)cc1]

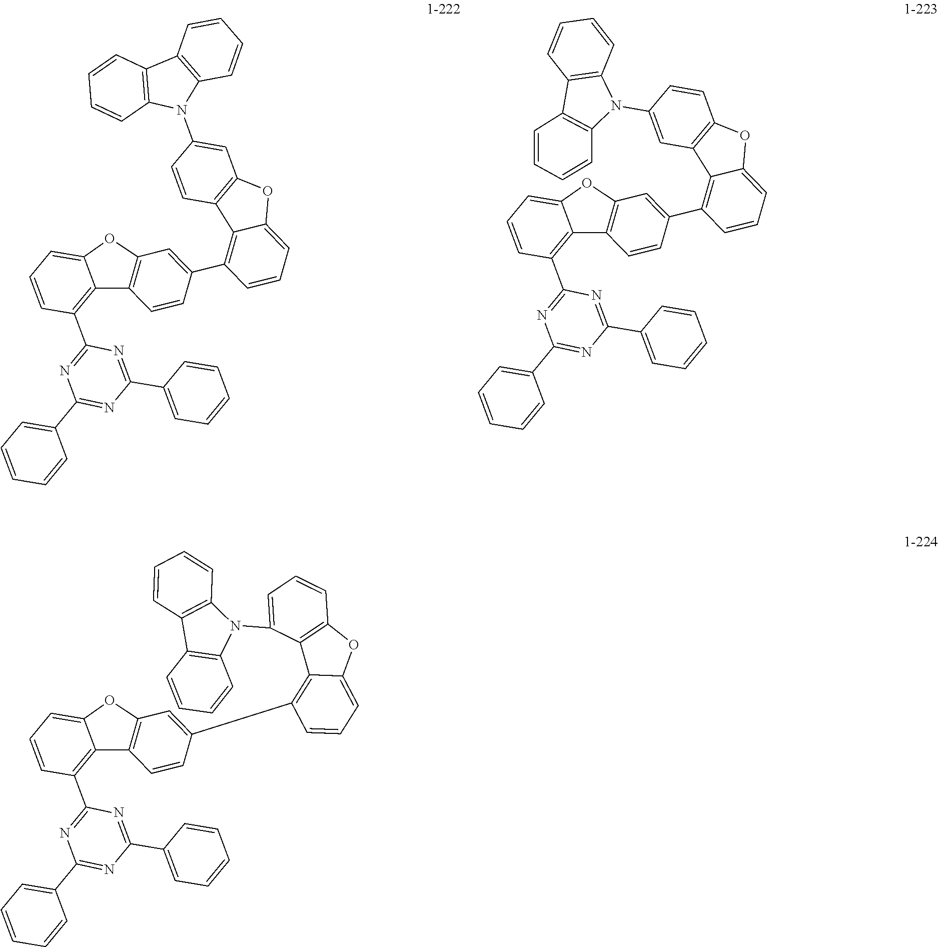 [SMILES: c1ccc(-c2nc(-c3ccccc3)nc(-c3cccc4oc5cc(-c6cccc7oc8cc(-n9c%10ccccc%10c%10ccccc%109)ccc8c67)ccc5c34)n2)cc1 ; c1ccc(-c2nc(-c3ccccc3)nc(-c3cccc4oc5cc(-c6cccc7oc8ccc(-n9c%10ccccc%10c%10ccccc%109)cc8c67)ccc5c34)n2)cc1 ; c1ccc(-c2nc(-c3ccccc3)nc(-c3cccc4oc5cc(-c6cccc7oc8cccc(-n9c%10ccccc%10c%10ccccc%109)c8c67)ccc5c34)n2)cc1]

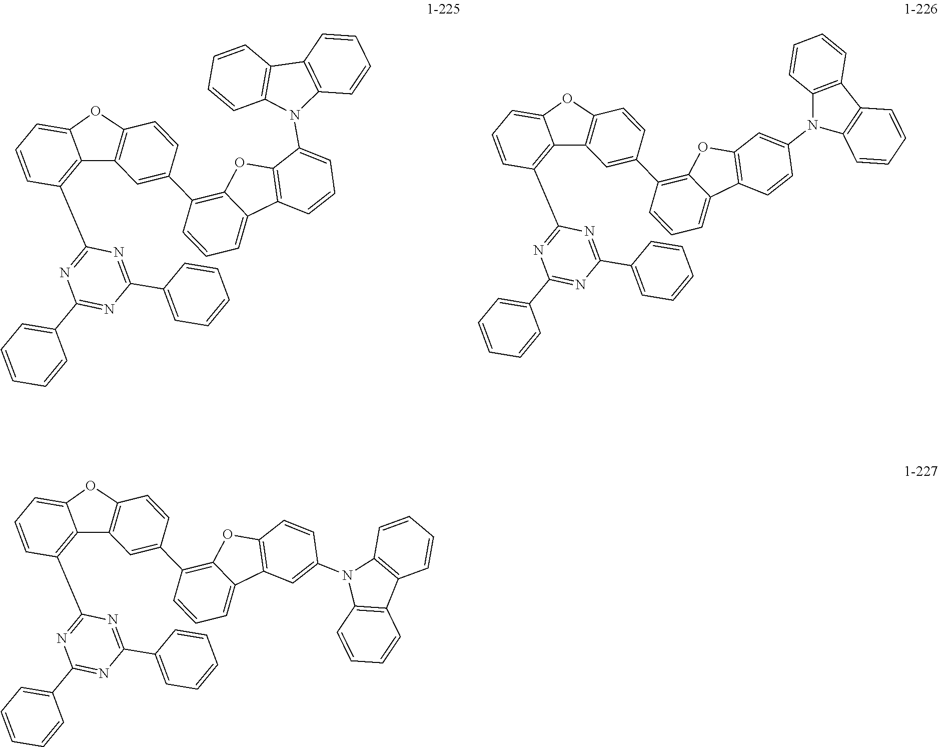 [SMILES: c1ccc(-c2nc(-c3ccccc3)nc(-c3cccc4oc5ccc(-c6cccc7c6oc6c(-n8c9ccccc9c9ccccc98)cccc67)cc5c34)n2)cc1 ; c1ccc(-c2nc(-c3ccccc3)nc(-c3cccc4oc5ccc(-c6cccc7c6oc6cc(-n8c9ccccc9c9ccccc98)ccc67)cc5c34)n2)cc1 ; c1ccc(-c2nc(-c3ccccc3)nc(-c3cccc4oc5ccc(-c6cccc7c6oc6ccc(-n8c9ccccc9c9ccccc98)cc67)cc5c34)n2)cc1]

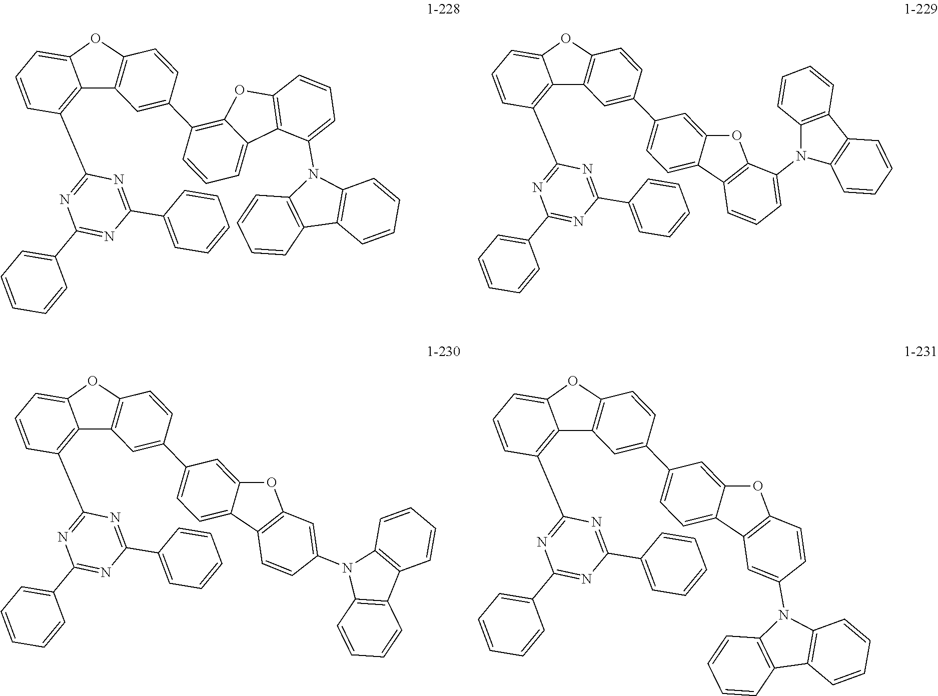 [SMILES: c1ccc(-c2nc(-c3ccccc3)nc(-c3cccc4oc5ccc(-c6cccc7c6oc6cccc(-n8c9ccccc9c9ccccc98)c67)cc5c34)n2)cc1 ; c1ccc(-c2nc(-c3ccccc3)nc(-c3cccc4oc5ccc(-c6ccc7c(c6)oc6c(-n8c9ccccc9c9ccccc98)cccc67)cc5c34)n2)cc1 ; c1ccc(-c2nc(-c3ccccc3)nc(-c3cccc4oc5ccc(-c6ccc7c(c6)oc6cc(-n8c9ccccc9c9ccccc98)ccc67)cc5c34)n2)cc1 ; c1ccc(-c2nc(-c3ccccc3)nc(-c3cccc4oc5ccc(-c6ccc7c(c6)oc6ccc(-n8c9ccccc9c9ccccc98)cc67)cc5c34)n2)cc1]

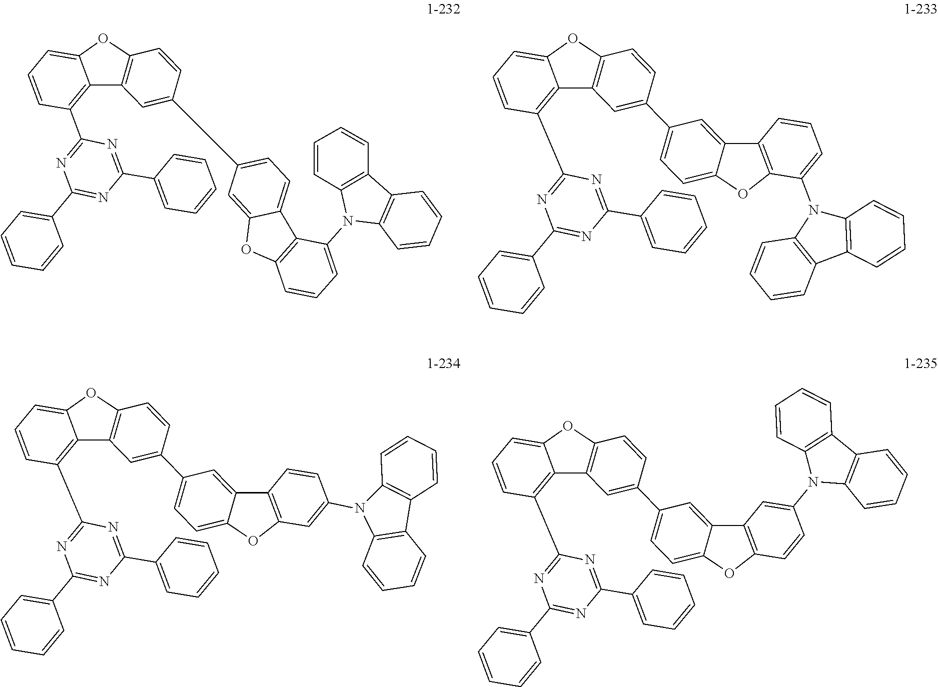 [SMILES: c1ccc(-c2nc(-c3ccccc3)nc(-c3cccc4oc5ccc(-c6ccc7c(c6)oc6cccc(-n8c9ccccc9c9ccccc98)c67)cc5c34)n2)cc1 ; c1ccc(-c2nc(-c3ccccc3)nc(-c3cccc4oc5ccc(-c6ccc7oc8c(-n9c%10ccccc%10c%10ccccc%109)cccc8c7c6)cc5c34)n2)cc1 ; c1ccc(-c2nc(-c3ccccc3)nc(-c3cccc4oc5ccc(-c6ccc7oc8cc(-n9c%10ccccc%10c%10ccccc%109)ccc8c7c6)cc5c34)n2)cc1 ; c1ccc(-c2nc(-c3ccccc3)nc(-c3cccc4oc5ccc(-c6ccc7oc8ccc(-n9c%10ccccc%10c%10ccccc%109)cc8c7c6)cc5c34)n2)cc1]

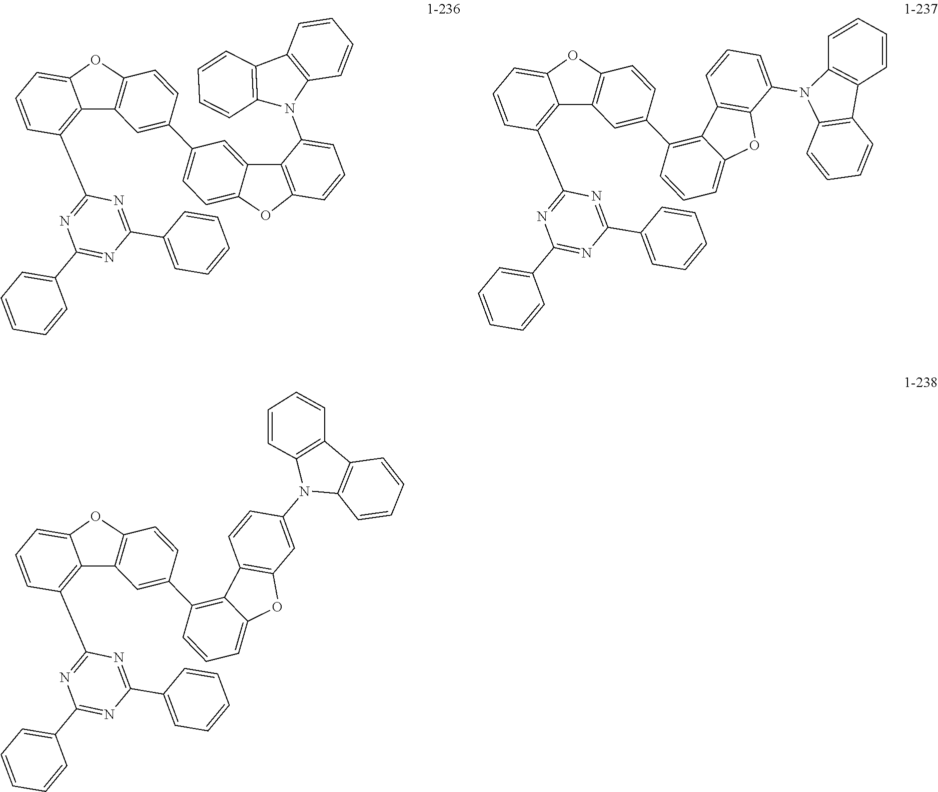 [SMILES: c1ccc(-c2nc(-c3ccccc3)nc(-c3cccc4oc5ccc(-c6ccc7oc8cccc(-n9c%10ccccc%10c%10ccccc%109)c8c7c6)cc5c34)n2)cc1 ; c1ccc(-c2nc(-c3ccccc3)nc(-c3cccc4oc5ccc(-c6cccc7oc8c(-n9c%10ccccc%10c%10ccccc%109)cccc8c67)cc5c34)n2)cc1 ; c1ccc(-c2nc(-c3ccccc3)nc(-c3cccc4oc5ccc(-c6cccc7oc8cc(-n9c%10ccccc%10c%10ccccc%109)ccc8c67)cc5c34)n2)cc1]

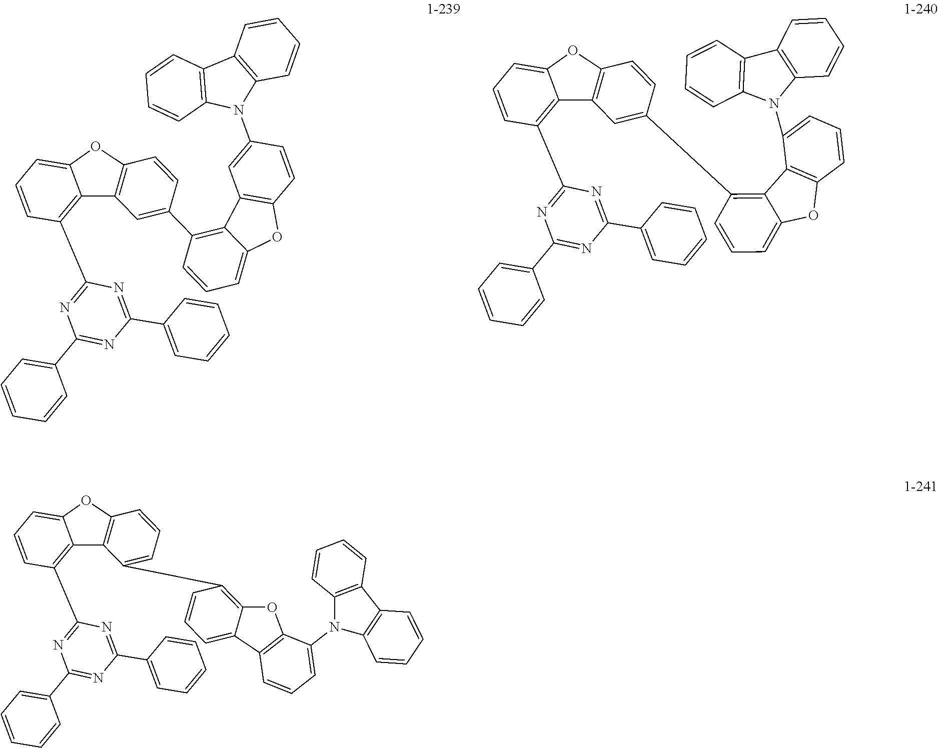 [SMILES: c1ccc(-c2nc(-c3ccccc3)nc(-c3cccc4oc5ccc(-c6cccc7oc8ccc(-n9c%10ccccc%10c%10ccccc%109)cc8c67)cc5c34)n2)cc1 ; c1ccc(-c2nc(-c3ccccc3)nc(-c3cccc4oc5ccc(-c6cccc7oc8cccc(-n9c%10ccccc%10c%10ccccc%109)c8c67)cc5c34)n2)cc1 ; c1ccc(-c2nc(-c3ccccc3)nc(-c3cccc4oc5cccc(-c6cccc7c6oc6c(-n8c9ccccc9c9ccccc98)cccc67)c5c34)n2)cc1]

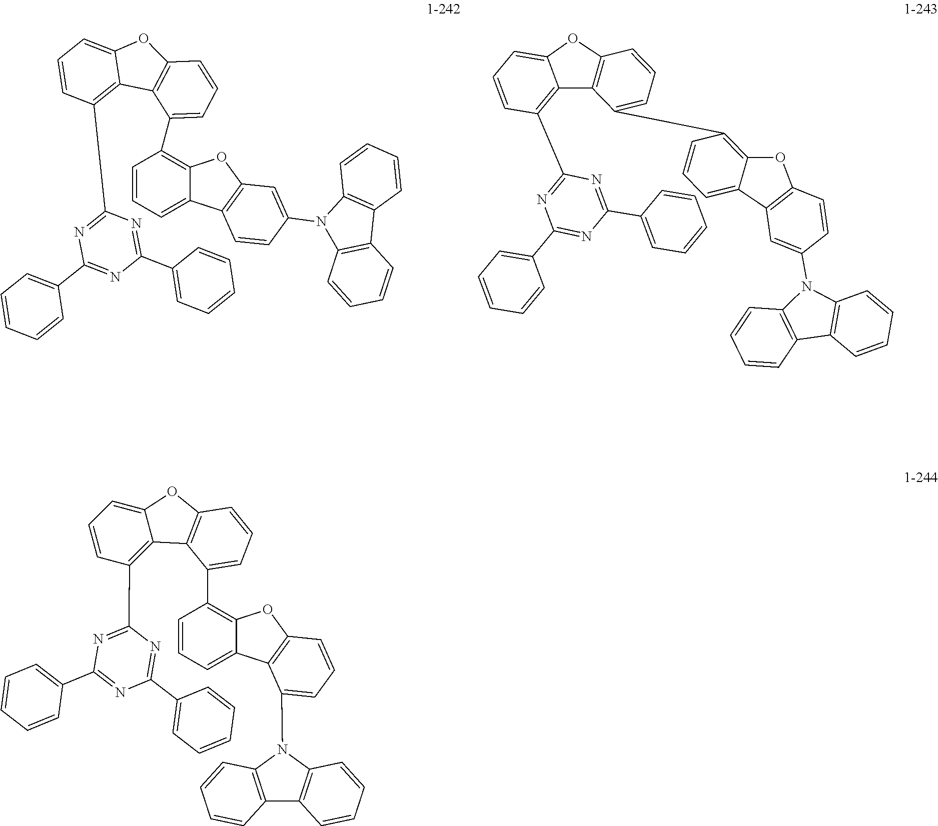 [SMILES: c1ccc(-c2nc(-c3ccccc3)nc(-c3cccc4oc5cccc(-c6cccc7c6oc6cc(-n8c9ccccc9c9ccccc98)ccc67)c5c34)n2)cc1 ; c1ccc(-c2nc(-c3ccccc3)nc(-c3cccc4oc5cccc(-c6cccc7c6oc6ccc(-n8c9ccccc9c9ccccc98)cc67)c5c34)n2)cc1 ; c1ccc(-c2nc(-c3ccccc3)nc(-c3cccc4oc5cccc(-c6cccc7c6oc6cccc(-n8c9ccccc9c9ccccc98)c67)c5c34)n2)cc1]

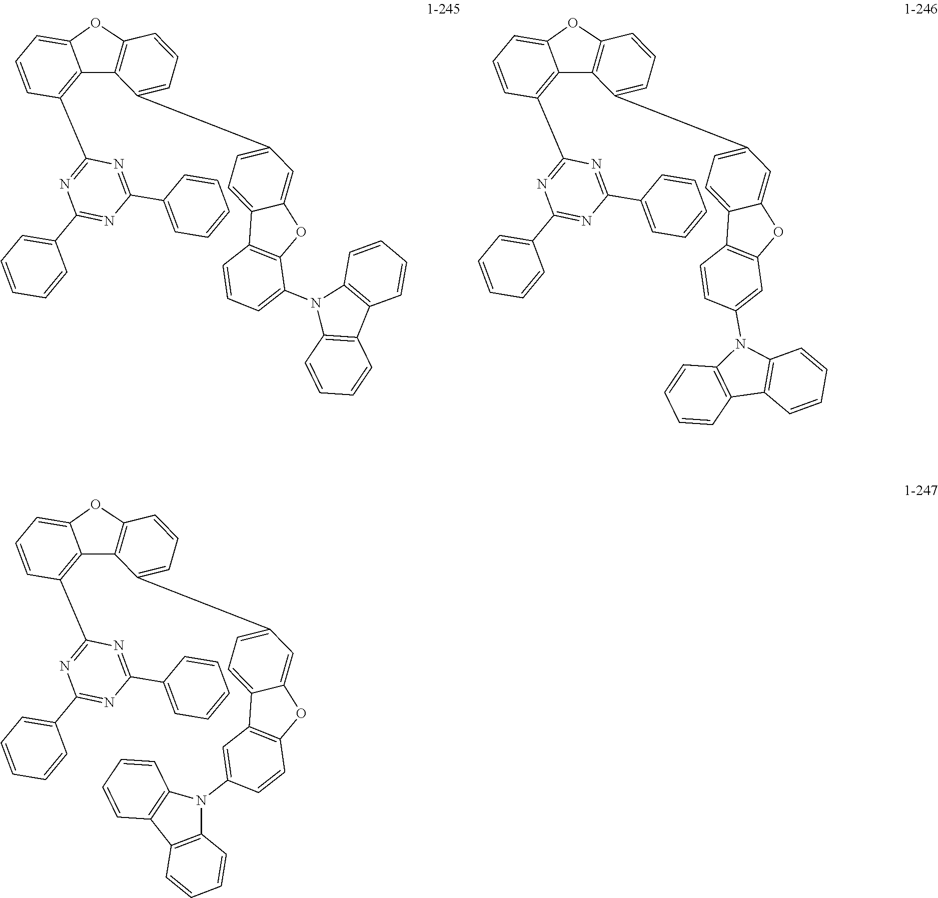 [SMILES: c1ccc(-c2nc(-c3ccccc3)nc(-c3cccc4oc5cccc(-c6ccc7c(c6)oc6c(-n8c9ccccc9c9ccccc98)cccc67)c5c34)n2)cc1 ; c1ccc(-c2nc(-c3ccccc3)nc(-c3cccc4oc5cccc(-c6ccc7c(c6)oc6cc(-n8c9ccccc9c9ccccc98)ccc67)c5c34)n2)cc1 ; c1ccc(-c2nc(-c3ccccc3)nc(-c3cccc4oc5cccc(-c6ccc7c(c6)oc6ccc(-n8c9ccccc9c9ccccc98)cc67)c5c34)n2)cc1]

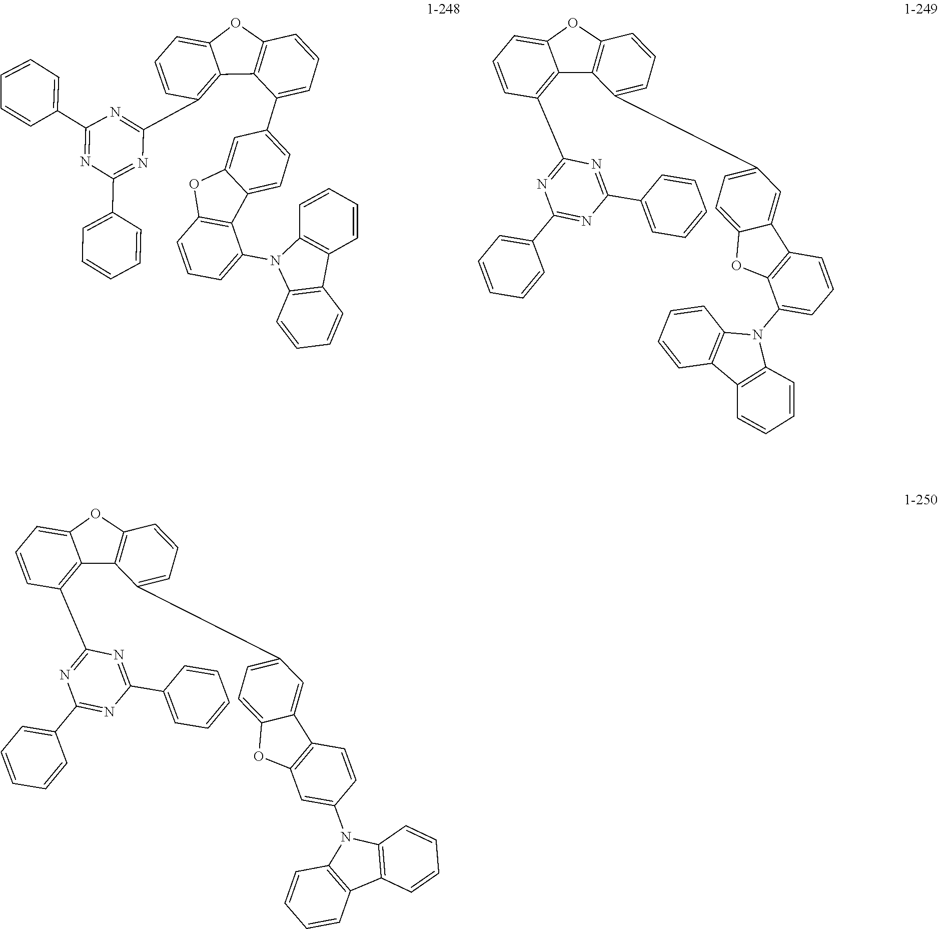 [SMILES: c1ccc(-c2nc(-c3ccccc3)nc(-c3cccc4oc5cccc(-c6ccc7c(c6)oc6cccc(-n8c9ccccc9c9ccccc98)c67)c5c34)n2)cc1 ; c1ccc(-c2nc(-c3ccccc3)nc(-c3cccc4oc5cccc(-c6ccc7oc8c(-n9c%10ccccc%10c%10ccccc%109)cccc8c7c6)c5c34)n2)cc1 ; c1ccc(-c2nc(-c3ccccc3)nc(-c3cccc4oc5cccc(-c6ccc7oc8cc(-n9c%10ccccc%10c%10ccccc%109)ccc8c7c6)c5c34)n2)cc1]

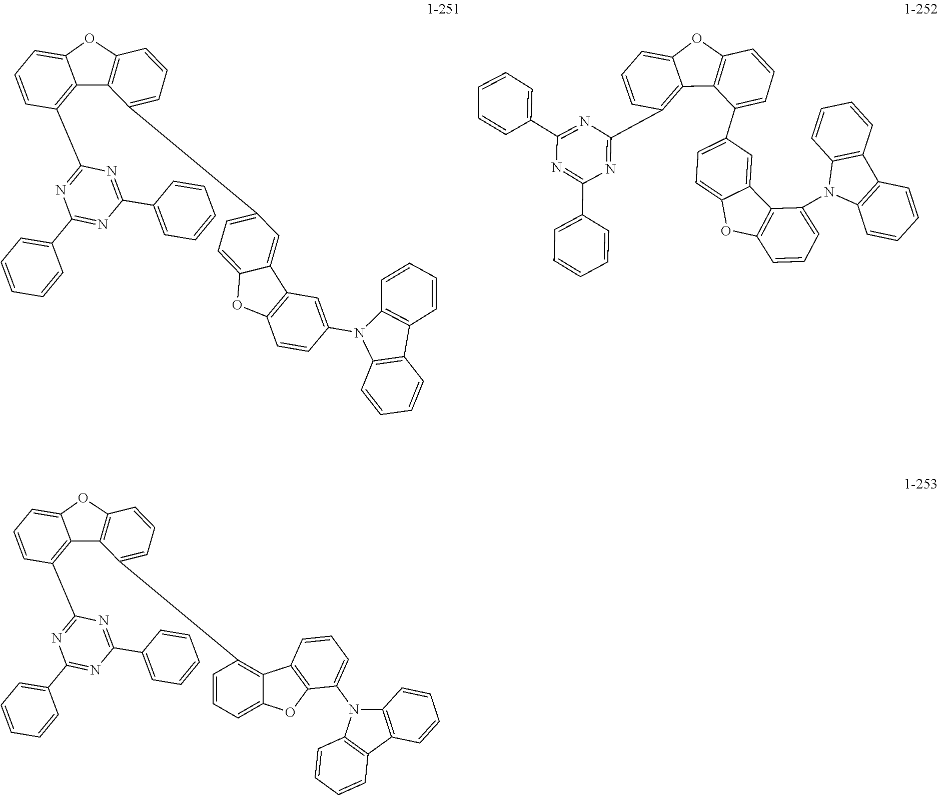 [SMILES: c1ccc(-c2nc(-c3ccccc3)nc(-c3cccc4oc5cccc(-c6ccc7oc8ccc(-n9c%10ccccc%10c%10ccccc%109)cc8c7c6)c5c34)n2)cc1 ; c1ccc(-c2nc(-c3ccccc3)nc(-c3cccc4oc5cccc(-c6ccc7oc8cccc(-n9c%10ccccc%10c%10ccccc%109)c8c7c6)c5c34)n2)cc1 ; c1ccc(-c2nc(-c3ccccc3)nc(-c3cccc4oc5cccc(-c6cccc7oc8c(-n9c%10ccccc%10c%10ccccc%109)cccc8c67)c5c34)n2)cc1]

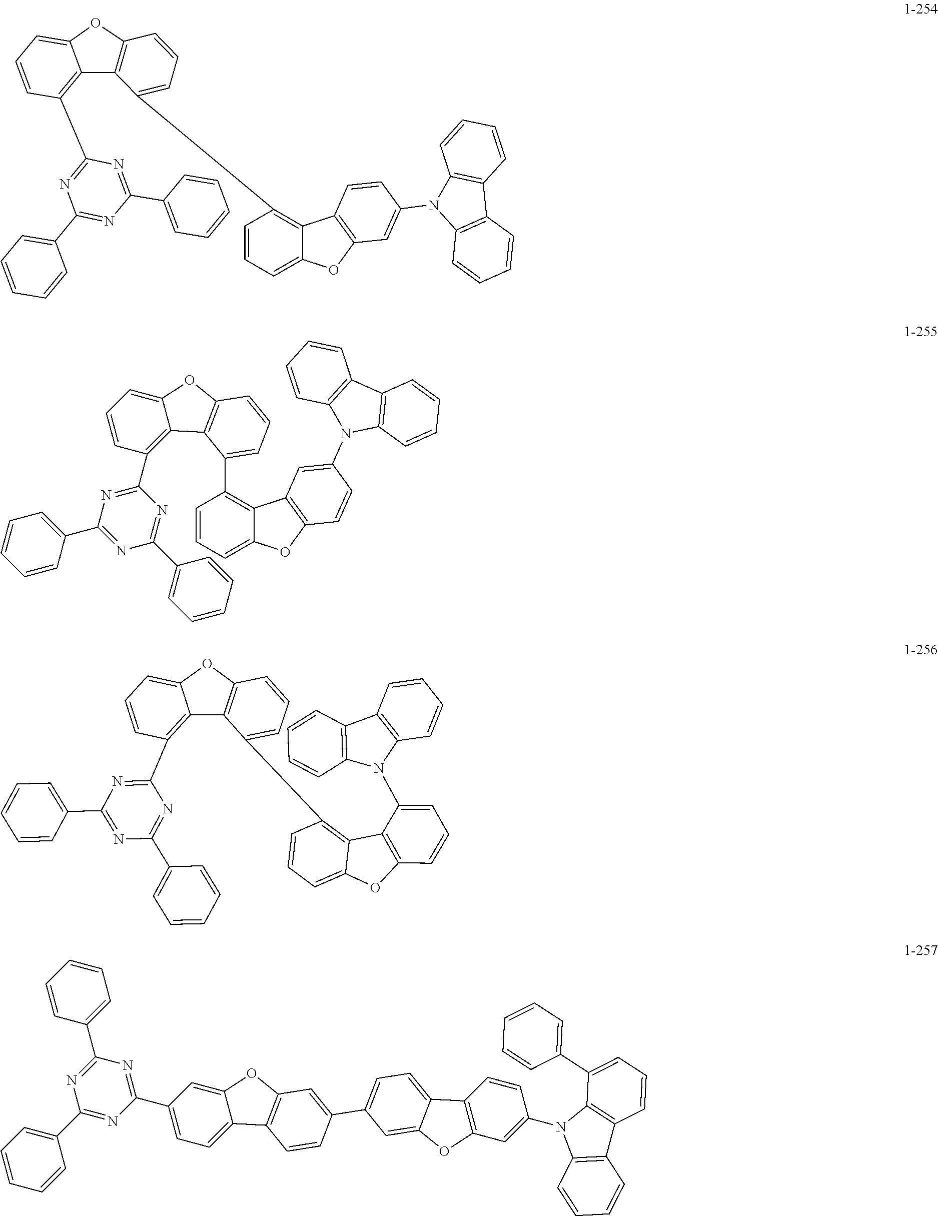

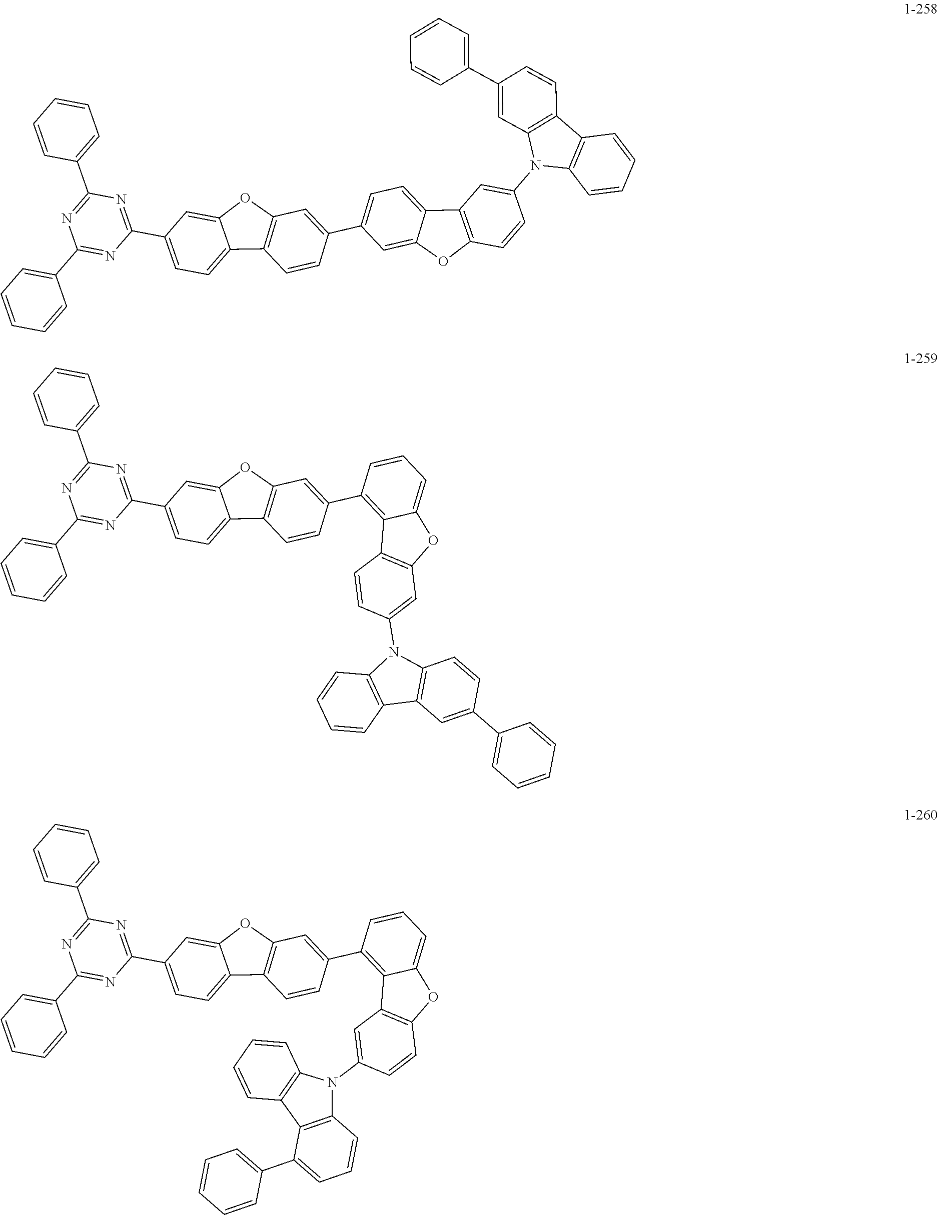

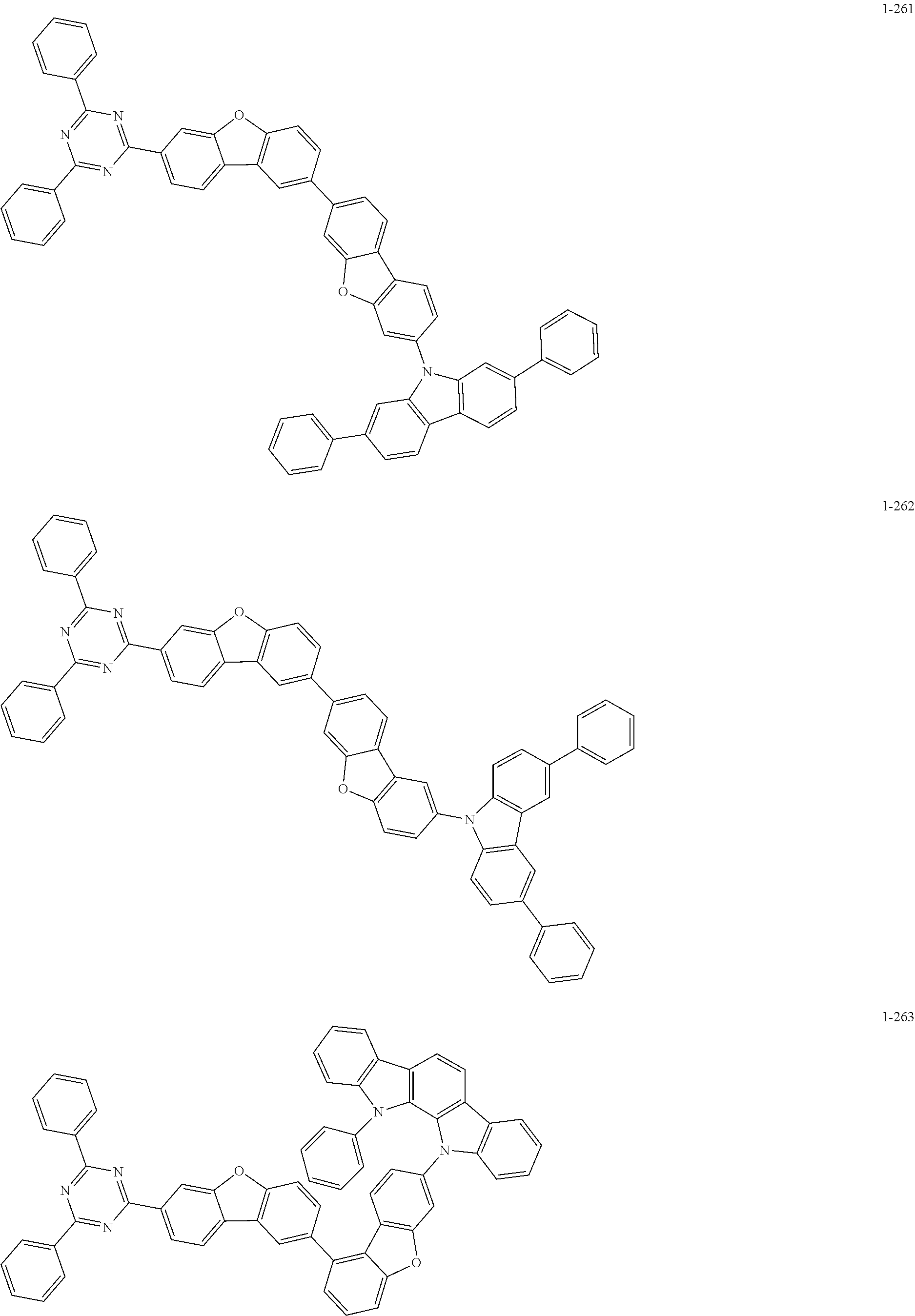 [SMILES: c1ccc(-c2ccc3c4ccc(-c5ccccc5)cc4n(-c4ccc5c(c4)oc4cc(-c6ccc7oc8cc(-c9nc(-c%10ccccc%10)nc(-c%10ccccc%10)n9)ccc8c7c6)ccc45)c3c2)cc1 ; c1ccc(-c2ccc3c(c2)c2cc(-c4ccccc4)ccc2n3-c2ccc3oc4cc(-c5ccc6oc7cc(-c8nc(-c9ccccc9)nc(-c9ccccc9)n8)ccc7c6c5)ccc4c3c2)cc1 ; c1ccc(-c2nc(-c3ccccc3)nc(-c3ccc4c(c3)oc3ccc(-c5cccc6oc7cc(-n8c9ccccc9c9ccc%10c%11ccccc%11n(-c%11ccccc%11)c%10c98)ccc7c56)cc34)n2)cc1]

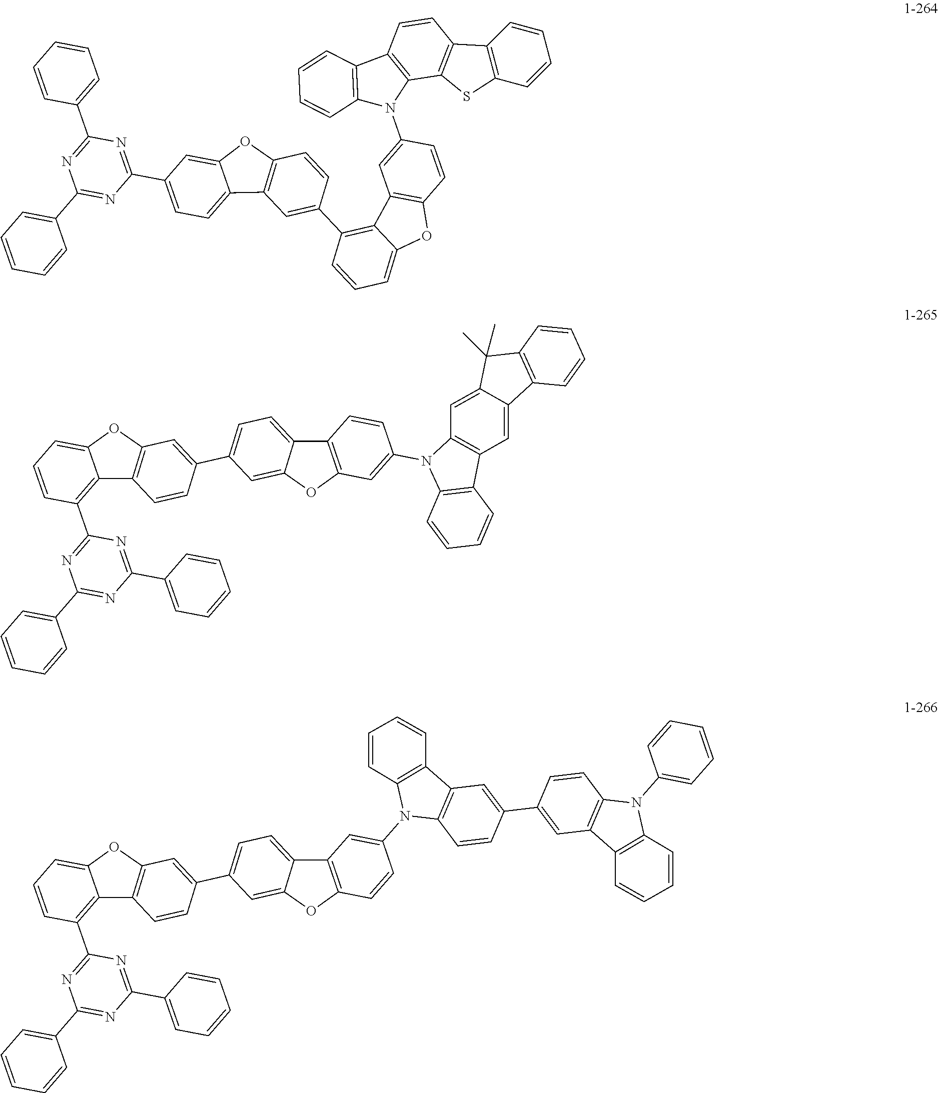 [SMILES: c1ccc(-c2nc(-c3ccccc3)nc(-c3ccc4c(c3)oc3ccc(-c5cccc6oc7ccc(-n8c9ccccc9c9ccc%10c%11ccccc%11sc%10c98)cc7c56)cc34)n2)cc1 ; CC1(C)c2ccccc2-c2cc3c4ccccc4n(-c4ccc5c(c4)oc4cc(-c6ccc7c(c6)oc6cccc(-c8nc(-c9ccccc9)nc(-c9ccccc9)n8)c67)ccc45)c3cc21 ; c1ccc(-c2nc(-c3ccccc3)nc(-c3cccc4oc5cc(-c6ccc7c(c6)oc6ccc(-n8c9ccccc9c9cc(-c%10ccc%11c(c%10)c%10ccccc%10n%11-c%10ccccc%10)ccc98)cc67)ccc5c34)n2)cc1]

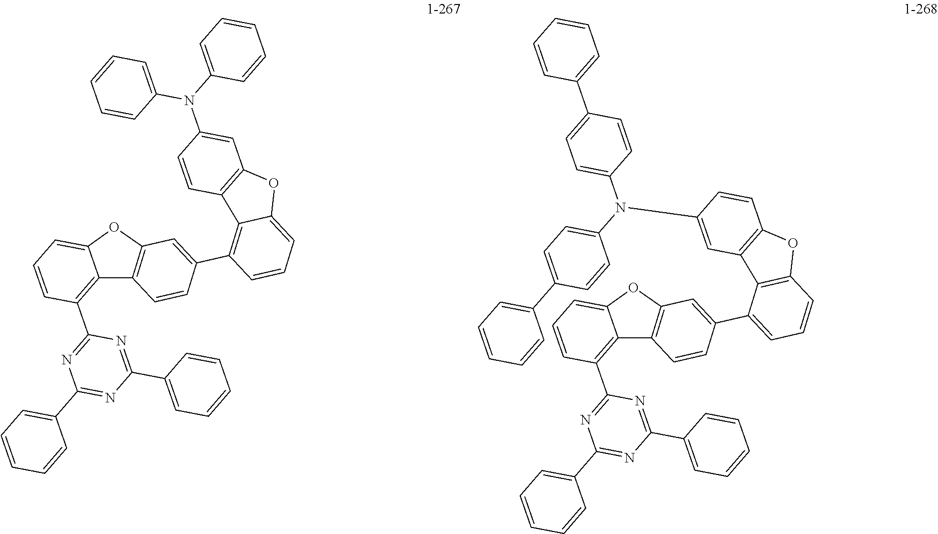 [SMILES: c1ccc(-c2nc(-c3ccccc3)nc(-c3cccc4oc5cc(-c6cccc7oc8cc(N(c9ccccc9)c9ccccc9)ccc8c67)ccc5c34)n2)cc1 ; c1ccc(-c2ccc(N(c3ccc(-c4ccccc4)cc3)c3ccc4oc5cccc(-c6ccc7c(c6)oc6cccc(-c8nc(-c9ccccc9)nc(-c9ccccc9)n8)c67)c5c4c3)cc2)cc1]

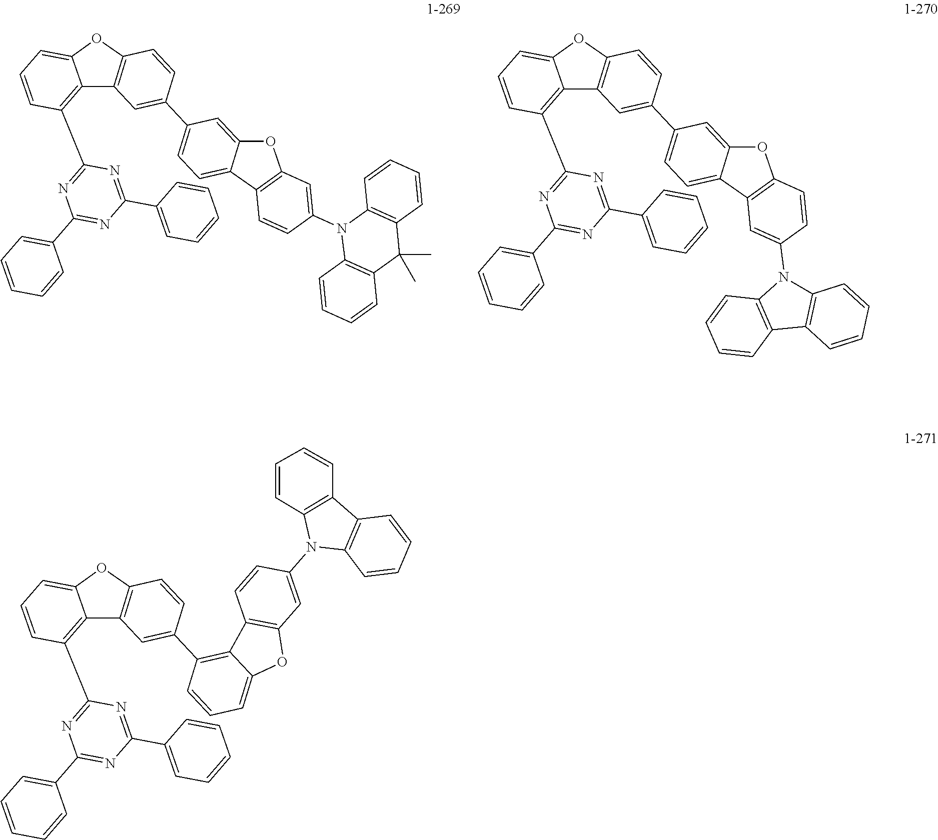 [SMILES: CC1(C)c2ccccc2N(c2ccc3c(c2)oc2cc(-c4ccc5oc6cccc(-c7nc(-c8ccccc8)nc(-c8ccccc8)n7)c6c5c4)ccc23)c2ccccc21 ; c1ccc(-c2nc(-c3ccccc3)nc(-c3cccc4oc5ccc(-c6ccc7c(c6)oc6ccc(-n8c9ccccc9c9ccccc98)cc67)cc5c34)n2)cc1 ; c1ccc(-c2nc(-c3ccccc3)nc(-c3cccc4oc5ccc(-c6cccc7oc8cc(-n9c%10ccccc%10c%10ccccc%109)ccc8c67)cc5c34)n2)cc1]

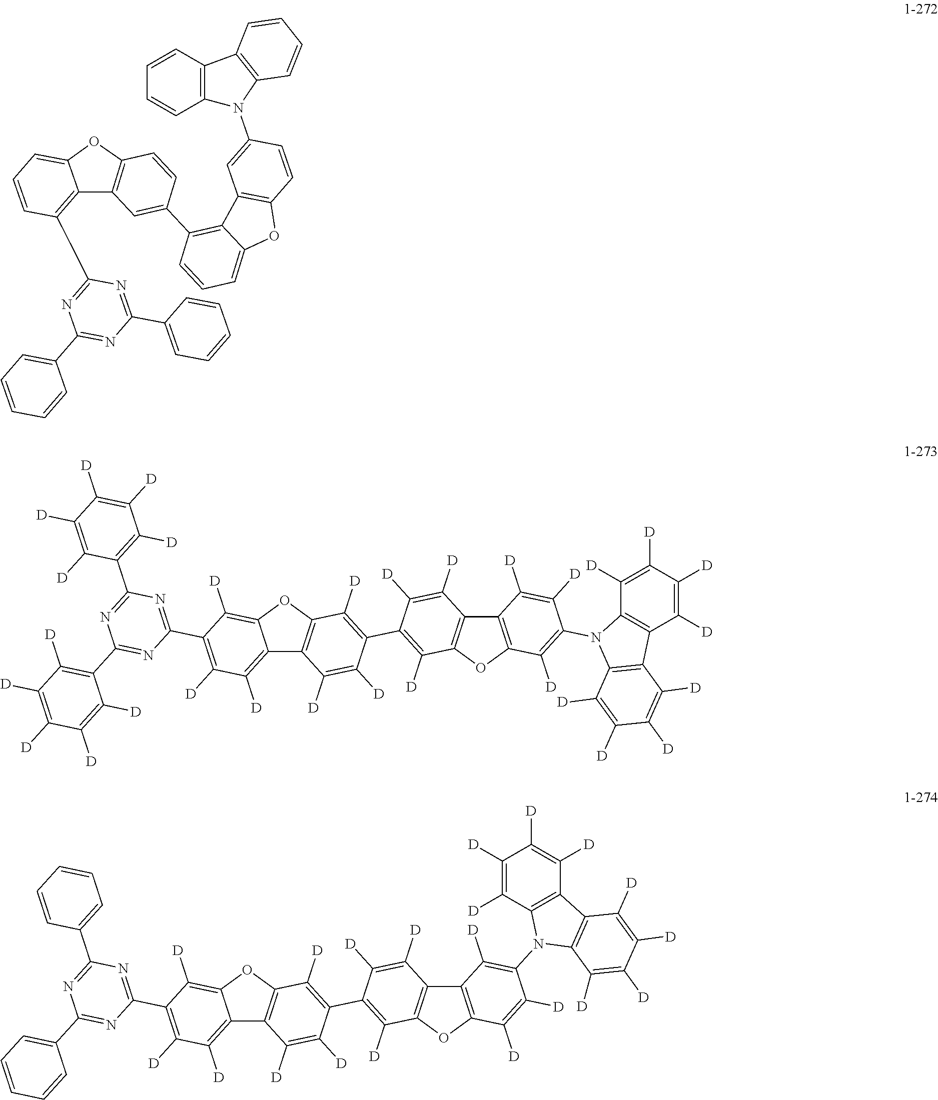

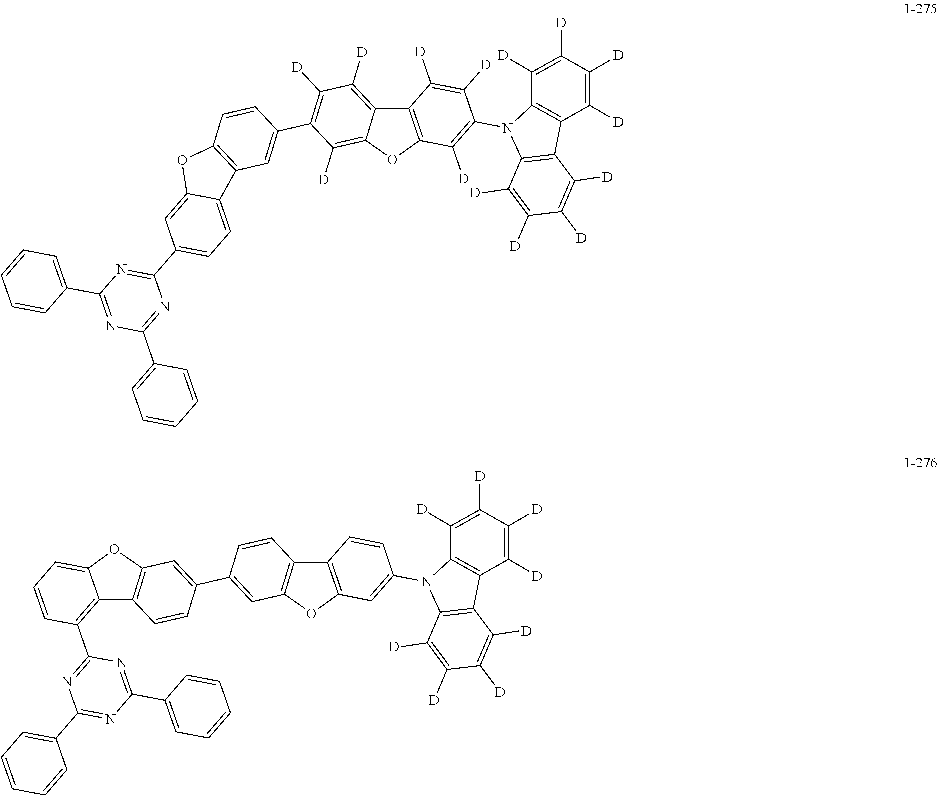 [SMILES: [2H]c1c(-c2ccc3oc4cc(-c5nc(-c6ccccc6)nc(-c6ccccc6)n5)ccc4c3c2)c([2H])c2oc3c([2H])c(-n4c5c([2H])c([2H])c([2H])c([2H])c5c5c([2H])c([2H])c([2H])c([2H])c54)c([2H])c([2H])c3c2c1[2H] ; [2H]c1c([2H])c([2H])c2c(c1[2H])c1c([2H])c([2H])c([2H])c([2H])c1n2-c1ccc2c(c1)oc1cc(-c3ccc4c(c3)oc3cccc(-c5nc(-c6ccccc6)nc(-c6ccccc6)n5)c34)ccc12]

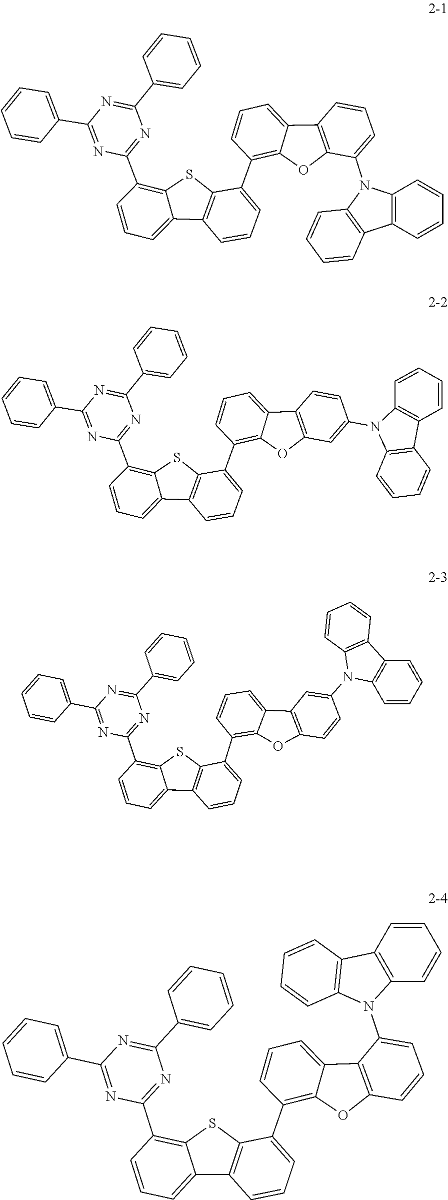 [SMILES: c1ccc(-c2nc(-c3ccccc3)nc(-c3cccc4c3sc3c(-c5cccc6c5oc5c(-n7c8ccccc8c8ccccc87)cccc56)cccc34)n2)cc1 ; c1ccc(-c2nc(-c3ccccc3)nc(-c3cccc4c3sc3c(-c5cccc6c5oc5cc(-n7c8ccccc8c8ccccc87)ccc56)cccc34)n2)cc1 ; c1ccc(-c2nc(-c3ccccc3)nc(-c3cccc4c3sc3c(-c5cccc6c5oc5ccc(-n7c8ccccc8c8ccccc87)cc56)cccc34)n2)cc1 ; c1ccc(-c2nc(-c3ccccc3)nc(-c3cccc4c3sc3c(-c5cccc6c5oc5cccc(-n7c8ccccc8c8ccccc87)c56)cccc34)n2)cc1]

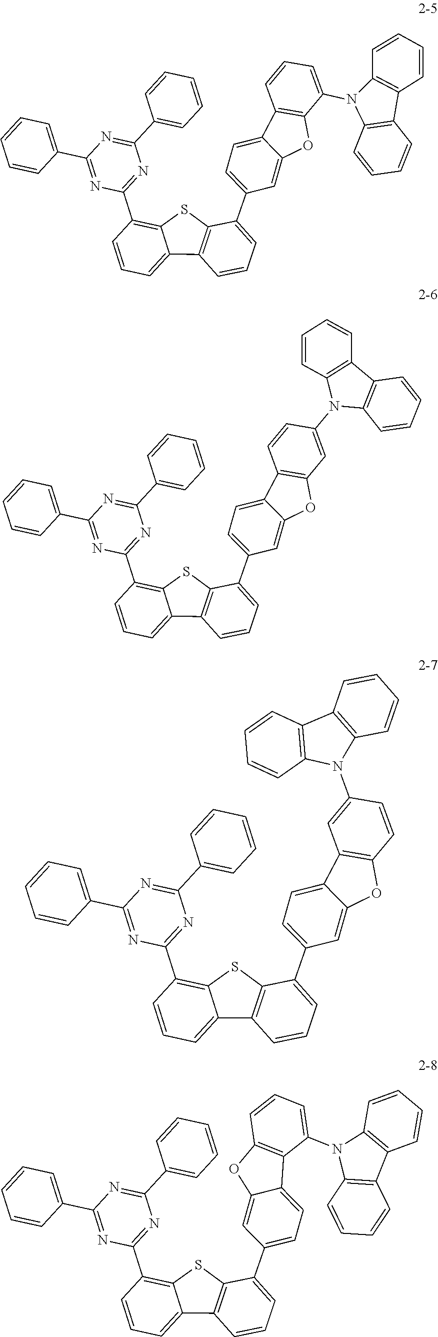 [SMILES: c1ccc(-c2nc(-c3ccccc3)nc(-c3cccc4c3sc3c(-c5ccc6c(c5)oc5c(-n7c8ccccc8c8ccccc87)cccc56)cccc34)n2)cc1 ; c1ccc(-c2nc(-c3ccccc3)nc(-c3cccc4c3sc3c(-c5ccc6c(c5)oc5cc(-n7c8ccccc8c8ccccc87)ccc56)cccc34)n2)cc1 ; c1ccc(-c2nc(-c3ccccc3)nc(-c3cccc4c3sc3c(-c5ccc6c(c5)oc5ccc(-n7c8ccccc8c8ccccc87)cc56)cccc34)n2)cc1 ; c1ccc(-c2nc(-c3ccccc3)nc(-c3cccc4c3sc3c(-c5ccc6c(c5)oc5cccc(-n7c8ccccc8c8ccccc87)c56)cccc34)n2)cc1]

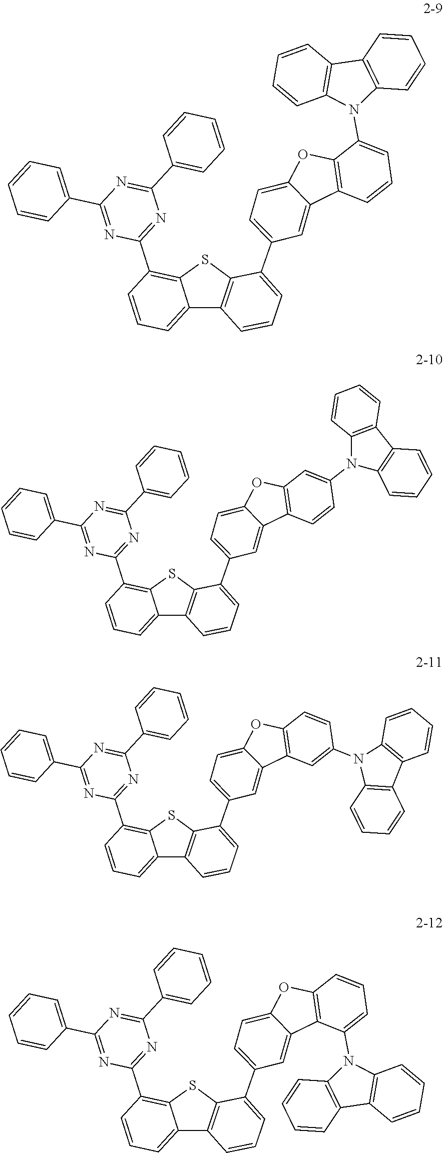 [SMILES: c1ccc(-c2nc(-c3ccccc3)nc(-c3cccc4c3sc3c(-c5ccc6oc7c(-n8c9ccccc9c9ccccc98)cccc7c6c5)cccc34)n2)cc1 ; c1ccc(-c2nc(-c3ccccc3)nc(-c3cccc4c3sc3c(-c5ccc6oc7cc(-n8c9ccccc9c9ccccc98)ccc7c6c5)cccc34)n2)cc1 ; c1ccc(-c2nc(-c3ccccc3)nc(-c3cccc4c3sc3c(-c5ccc6oc7ccc(-n8c9ccccc9c9ccccc98)cc7c6c5)cccc34)n2)cc1 ; c1ccc(-c2nc(-c3ccccc3)nc(-c3cccc4c3sc3c(-c5ccc6oc7cccc(-n8c9ccccc9c9ccccc98)c7c6c5)cccc34)n2)cc1]

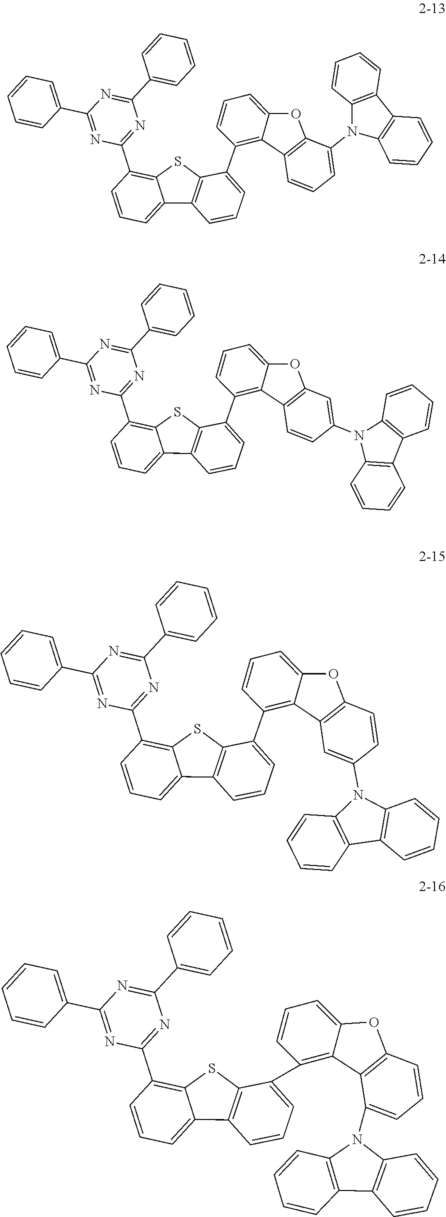 [SMILES: c1ccc(-c2nc(-c3ccccc3)nc(-c3cccc4c3sc3c(-c5cccc6oc7c(-n8c9ccccc9c9ccccc98)cccc7c56)cccc34)n2)cc1 ; c1ccc(-c2nc(-c3ccccc3)nc(-c3cccc4c3sc3c(-c5cccc6oc7cc(-n8c9ccccc9c9ccccc98)ccc7c56)cccc34)n2)cc1 ; c1ccc(-c2nc(-c3ccccc3)nc(-c3cccc4c3sc3c(-c5cccc6oc7ccc(-n8c9ccccc9c9ccccc98)cc7c56)cccc34)n2)cc1 ; c1ccc(-c2nc(-c3ccccc3)nc(-c3cccc4c3sc3c(-c5cccc6oc7cccc(-n8c9ccccc9c9ccccc98)c7c56)cccc34)n2)cc1]

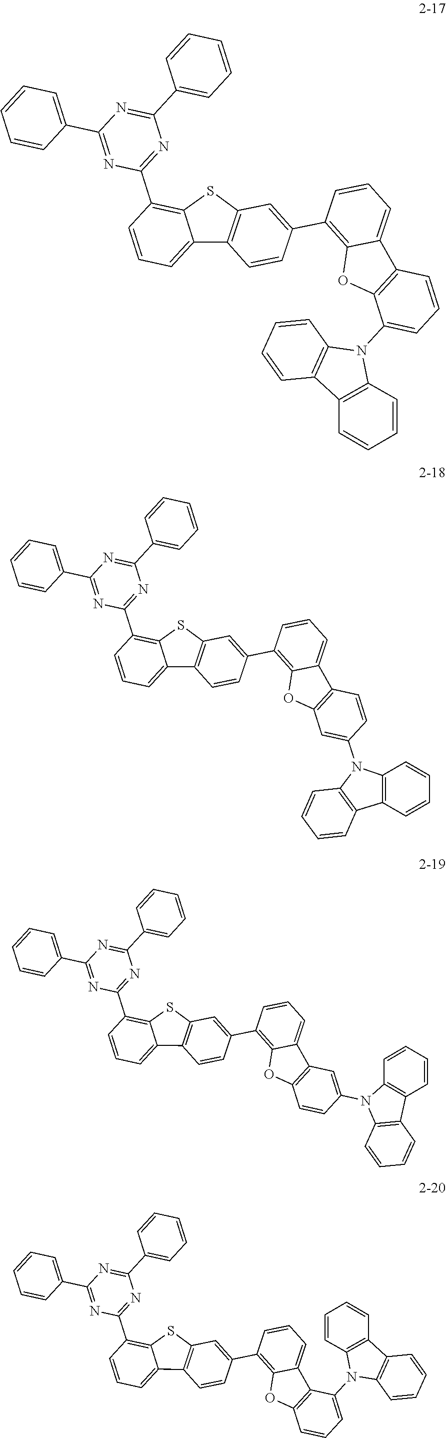 [SMILES: c1ccc(-c2nc(-c3ccccc3)nc(-c3cccc4c3sc3cc(-c5cccc6c5oc5c(-n7c8ccccc8c8ccccc87)cccc56)ccc34)n2)cc1 ; c1ccc(-c2nc(-c3ccccc3)nc(-c3cccc4c3sc3cc(-c5cccc6c5oc5cc(-n7c8ccccc8c8ccccc87)ccc56)ccc34)n2)cc1 ; c1ccc(-c2nc(-c3ccccc3)nc(-c3cccc4c3sc3cc(-c5cccc6c5oc5ccc(-n7c8ccccc8c8ccccc87)cc56)ccc34)n2)cc1 ; c1ccc(-c2nc(-c3ccccc3)nc(-c3cccc4c3sc3cc(-c5cccc6c5oc5cccc(-n7c8ccccc8c8ccccc87)c56)ccc34)n2)cc1]

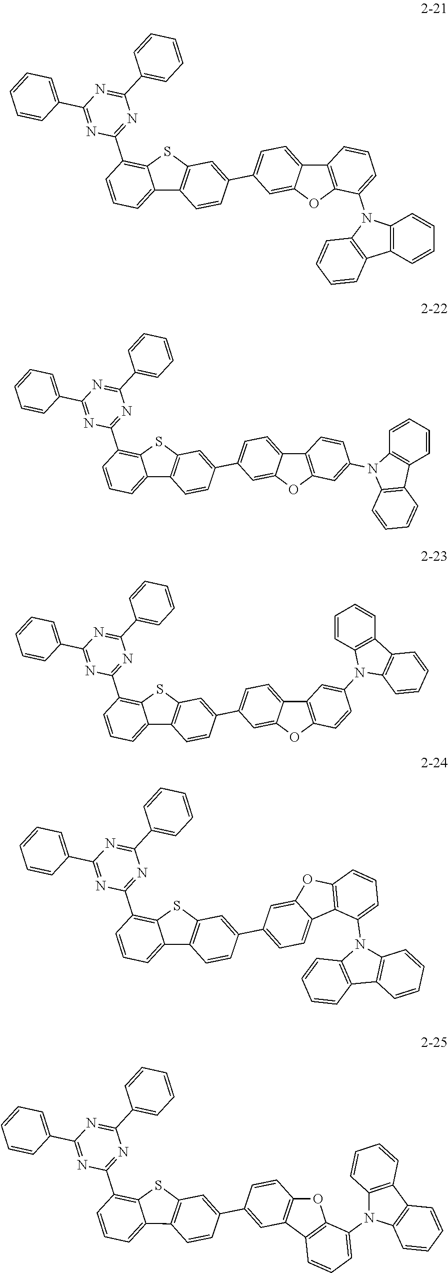 [SMILES: c1ccc(-c2nc(-c3ccccc3)nc(-c3cccc4c3sc3cc(-c5ccc6c(c5)oc5c(-n7c8ccccc8c8ccccc87)cccc56)ccc34)n2)cc1 ; c1ccc(-c2nc(-c3ccccc3)nc(-c3cccc4c3sc3cc(-c5ccc6c(c5)oc5cc(-n7c8ccccc8c8ccccc87)ccc56)ccc34)n2)cc1 ; c1ccc(-c2nc(-c3ccccc3)nc(-c3cccc4c3sc3cc(-c5ccc6c(c5)oc5ccc(-n7c8ccccc8c8ccccc87)cc56)ccc34)n2)cc1 ; c1ccc(-c2nc(-c3ccccc3)nc(-c3cccc4c3sc3cc(-c5ccc6c(c5)oc5cccc(-n7c8ccccc8c8ccccc87)c56)ccc34)n2)cc1 ; c1ccc(-c2nc(-c3ccccc3)nc(-c3cccc4c3sc3cc(-c5ccc6oc7c(-n8c9ccccc9c9ccccc98)cccc7c6c5)ccc34)n2)cc1]

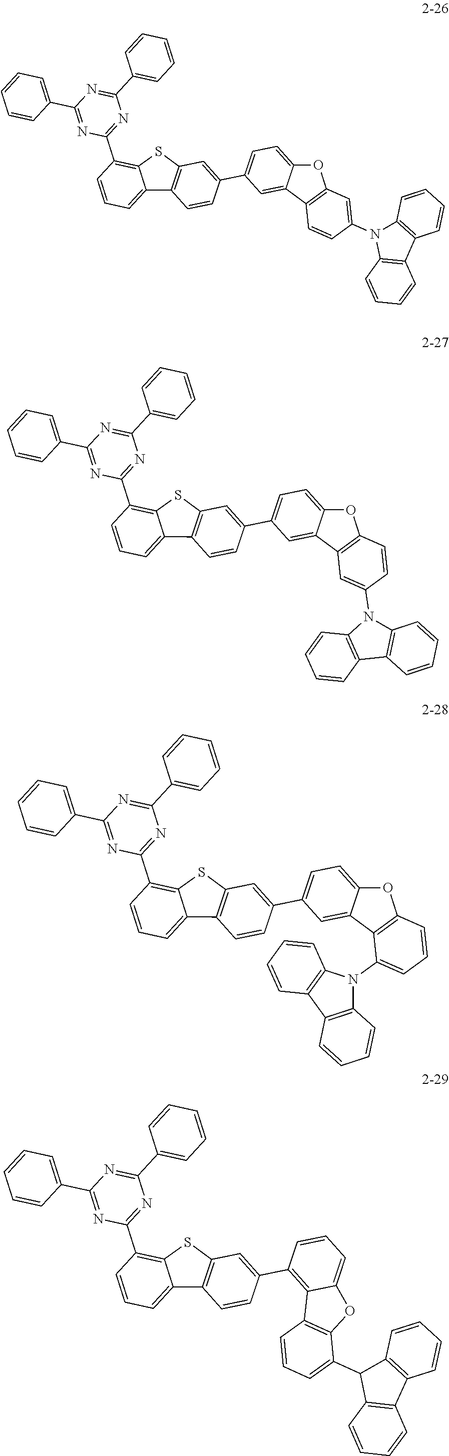 [SMILES: c1ccc(-c2nc(-c3ccccc3)nc(-c3cccc4c3sc3cc(-c5ccc6oc7cc(-n8c9ccccc9c9ccccc98)ccc7c6c5)ccc34)n2)cc1 ; c1ccc(-c2nc(-c3ccccc3)nc(-c3cccc4c3sc3cc(-c5ccc6oc7ccc(-n8c9ccccc9c9ccccc98)cc7c6c5)ccc34)n2)cc1 ; c1ccc(-c2nc(-c3ccccc3)nc(-c3cccc4c3sc3cc(-c5ccc6oc7cccc(-n8c9ccccc9c9ccccc98)c7c6c5)ccc34)n2)cc1 ; c1ccc(-c2nc(-c3ccccc3)nc(-c3cccc4c3sc3cc(-c5cccc6oc7c(C8c9ccccc9-c9ccccc98)cccc7c56)ccc34)n2)cc1]

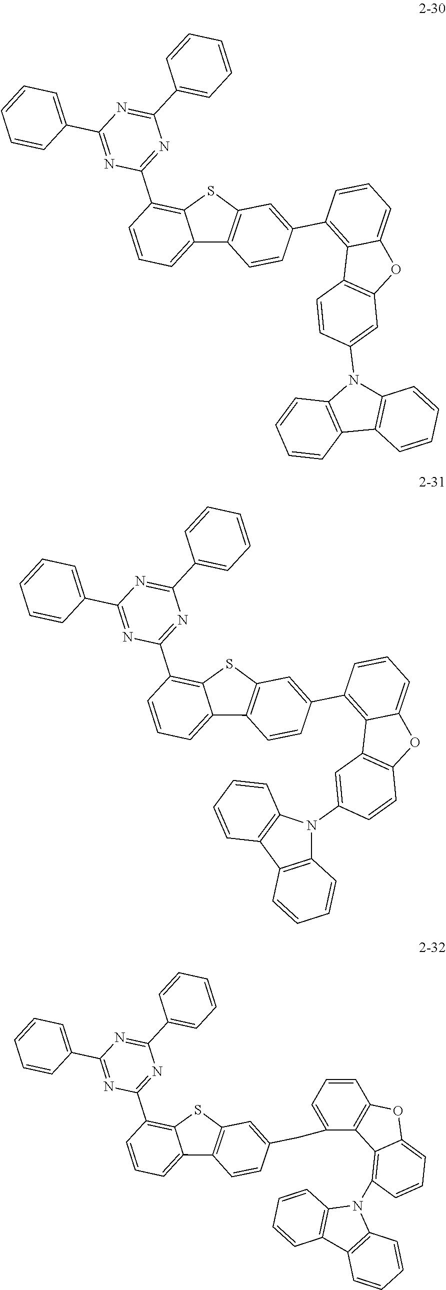 [SMILES: c1ccc(-c2nc(-c3ccccc3)nc(-c3cccc4c3sc3cc(-c5cccc6oc7cc(-n8c9ccccc9c9ccccc98)ccc7c56)ccc34)n2)cc1 ; c1ccc(-c2nc(-c3ccccc3)nc(-c3cccc4c3sc3cc(-c5cccc6oc7ccc(-n8c9ccccc9c9ccccc98)cc7c56)ccc34)n2)cc1 ; c1ccc(-c2nc(-c3ccccc3)nc(-c3cccc4c3sc3cc(Cc5cccc6oc7cccc(-n8c9ccccc9c9ccccc98)c7c56)ccc34)n2)cc1]

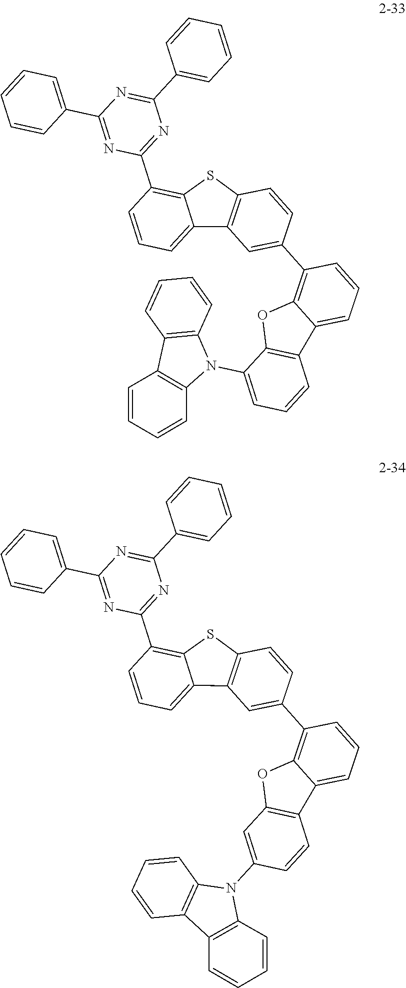 [SMILES: c1ccc(-c2nc(-c3ccccc3)nc(-c3cccc4c3sc3ccc(-c5cccc6c5oc5c(-n7c8ccccc8c8ccccc87)cccc56)cc34)n2)cc1 ; c1ccc(-c2nc(-c3ccccc3)nc(-c3cccc4c3sc3ccc(-c5cccc6c5oc5cc(-n7c8ccccc8c8ccccc87)ccc56)cc34)n2)cc1]

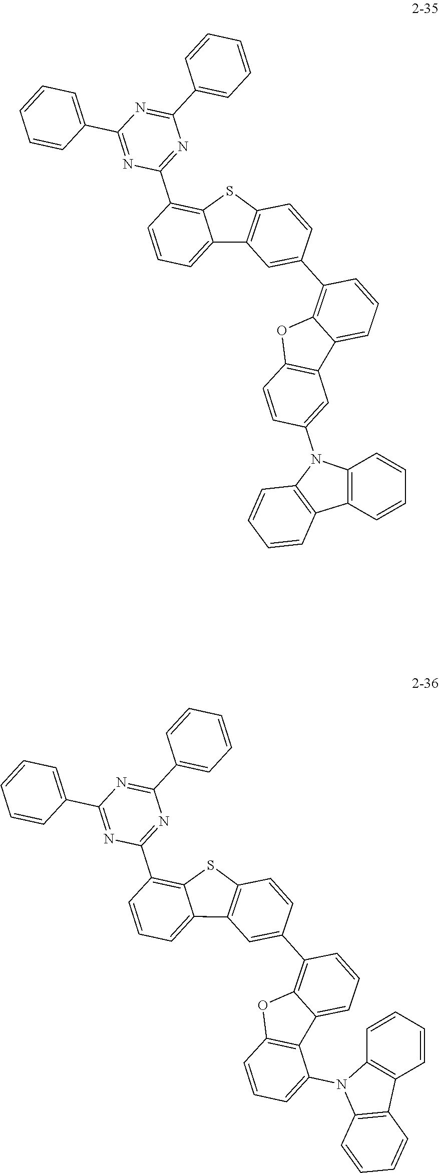 [SMILES: c1ccc(-c2nc(-c3ccccc3)nc(-c3cccc4c3sc3ccc(-c5cccc6c5oc5ccc(-n7c8ccccc8c8ccccc87)cc56)cc34)n2)cc1 ; c1ccc(-c2nc(-c3ccccc3)nc(-c3cccc4c3sc3ccc(-c5cccc6c5oc5cccc(-n7c8ccccc8c8ccccc87)c56)cc34)n2)cc1]

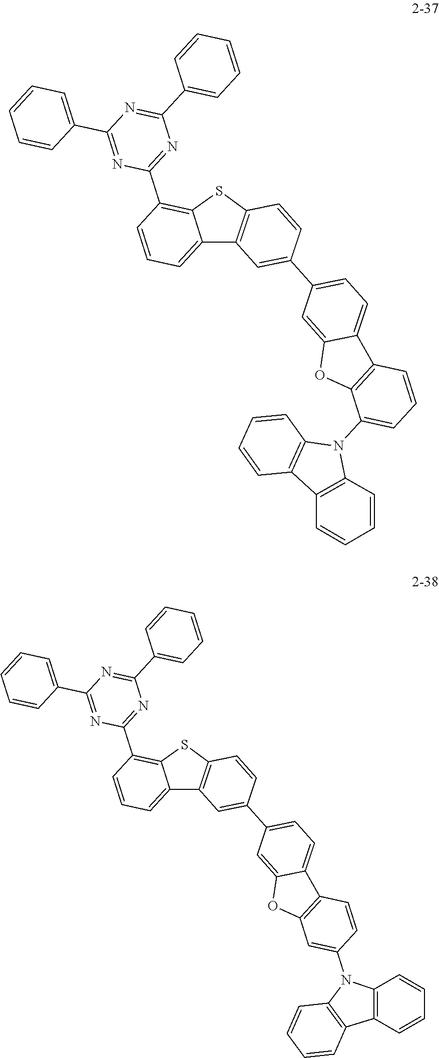 [SMILES: c1ccc(-c2nc(-c3ccccc3)nc(-c3cccc4c3sc3ccc(-c5ccc6c(c5)oc5c(-n7c8ccccc8c8ccccc87)cccc56)cc34)n2)cc1 ; c1ccc(-c2nc(-c3ccccc3)nc(-c3cccc4c3sc3ccc(-c5ccc6c(c5)oc5cc(-n7c8ccccc8c8ccccc87)ccc56)cc34)n2)cc1]

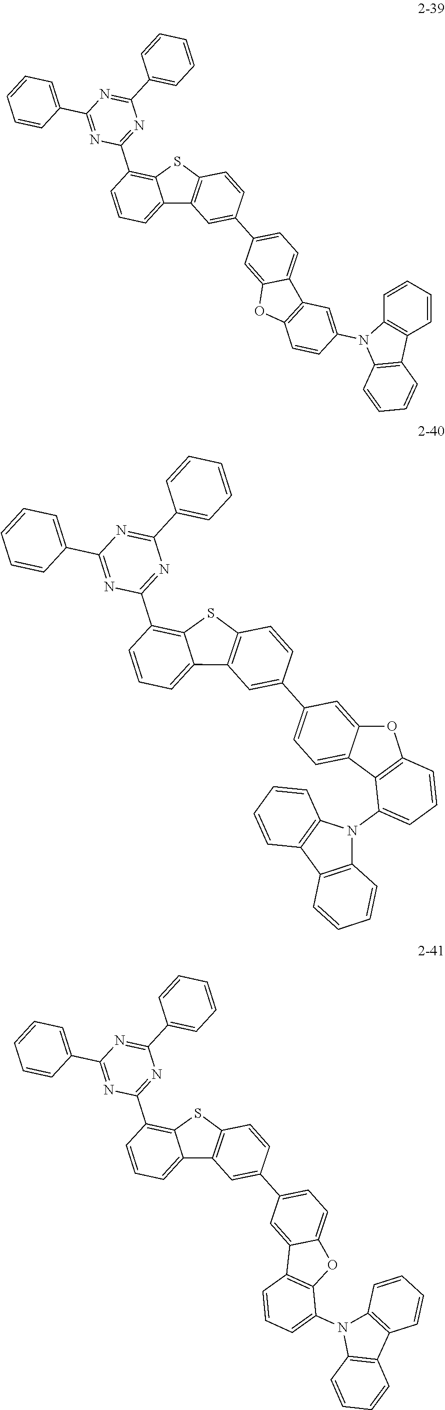 [SMILES: c1ccc(-c2nc(-c3ccccc3)nc(-c3cccc4c3sc3ccc(-c5ccc6c(c5)oc5ccc(-n7c8ccccc8c8ccccc87)cc56)cc34)n2)cc1 ; c1ccc(-c2nc(-c3ccccc3)nc(-c3cccc4c3sc3ccc(-c5ccc6c(c5)oc5cccc(-n7c8ccccc8c8ccccc87)c56)cc34)n2)cc1 ; c1ccc(-c2nc(-c3ccccc3)nc(-c3cccc4c3sc3ccc(-c5ccc6oc7c(-n8c9ccccc9c9ccccc98)cccc7c6c5)cc34)n2)cc1]

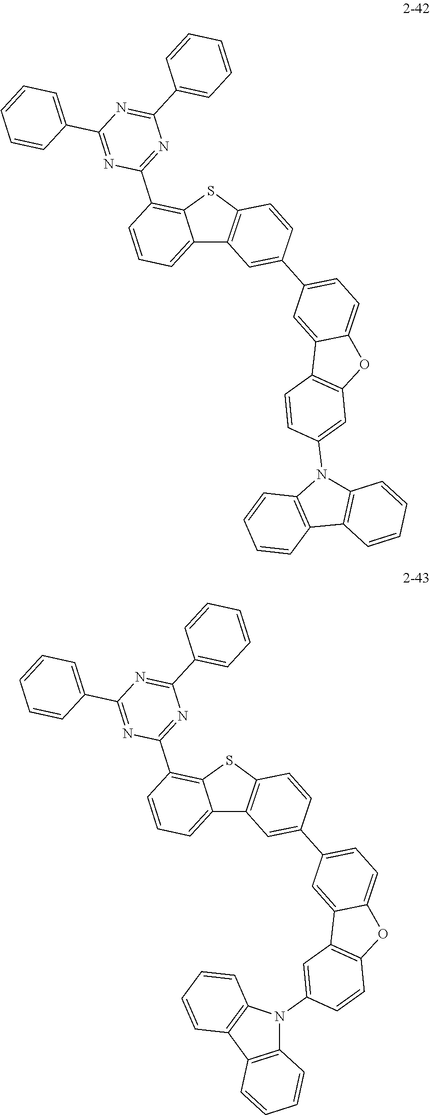 [SMILES: c1ccc(-c2nc(-c3ccccc3)nc(-c3cccc4c3sc3ccc(-c5ccc6oc7cc(-n8c9ccccc9c9ccccc98)ccc7c6c5)cc34)n2)cc1 ; c1ccc(-c2nc(-c3ccccc3)nc(-c3cccc4c3sc3ccc(-c5ccc6oc7ccc(-n8c9ccccc9c9ccccc98)cc7c6c5)cc34)n2)cc1]

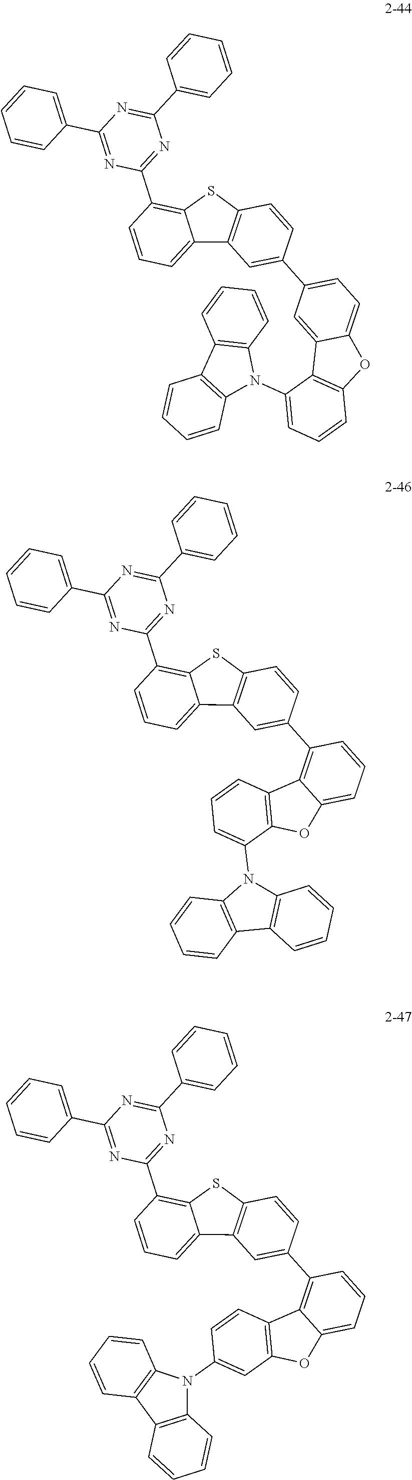 [SMILES: c1ccc(-c2nc(-c3ccccc3)nc(-c3cccc4c3sc3ccc(-c5ccc6oc7cccc(-n8c9ccccc9c9ccccc98)c7c6c5)cc34)n2)cc1 ; c1ccc(-c2nc(-c3ccccc3)nc(-c3cccc4c3sc3ccc(-c5cccc6oc7c(-n8c9ccccc9c9ccccc98)cccc7c56)cc34)n2)cc1 ; c1ccc(-c2nc(-c3ccccc3)nc(-c3cccc4c3sc3ccc(-c5cccc6oc7cc(-n8c9ccccc9c9ccccc98)ccc7c56)cc34)n2)cc1]

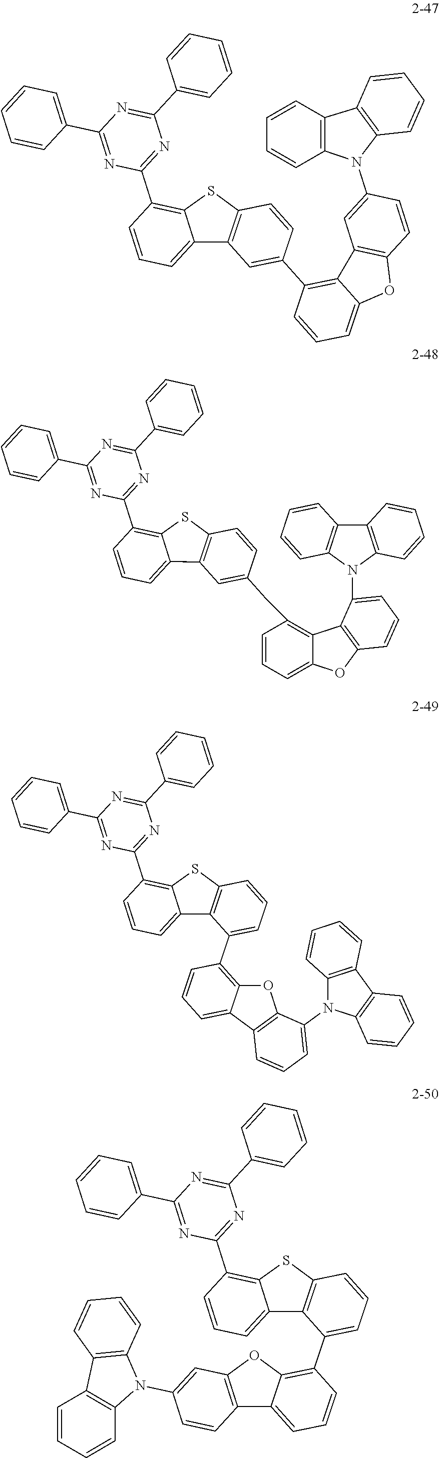

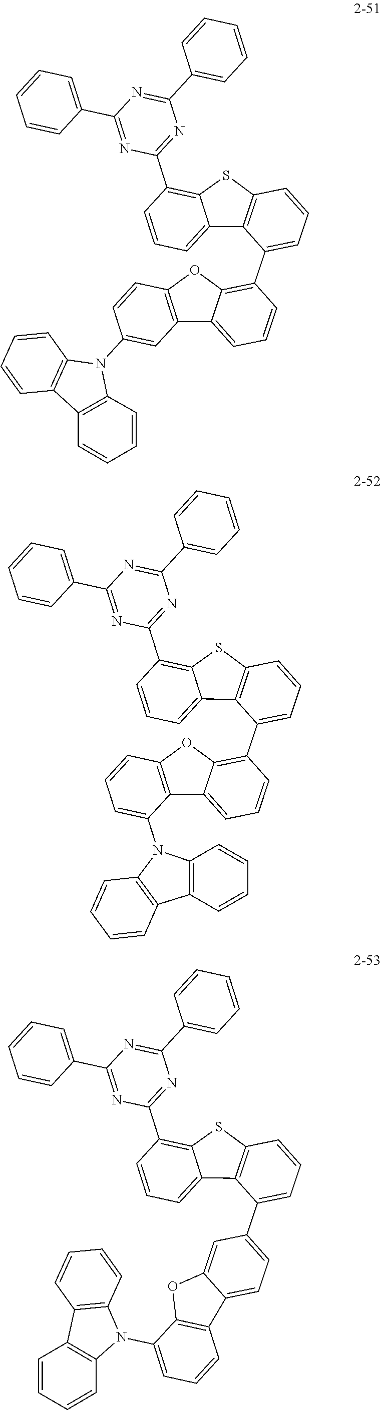 [SMILES: c1ccc(-c2nc(-c3ccccc3)nc(-c3cccc4c3sc3cccc(-c5cccc6c5oc5ccc(-n7c8ccccc8c8ccccc87)cc56)c34)n2)cc1 ; c1ccc(-c2nc(-c3ccccc3)nc(-c3cccc4c3sc3cccc(-c5cccc6c5oc5cccc(-n7c8ccccc8c8ccccc87)c56)c34)n2)cc1 ; c1ccc(-c2nc(-c3ccccc3)nc(-c3cccc4c3sc3cccc(-c5ccc6c(c5)oc5c(-n7c8ccccc8c8ccccc87)cccc56)c34)n2)cc1]

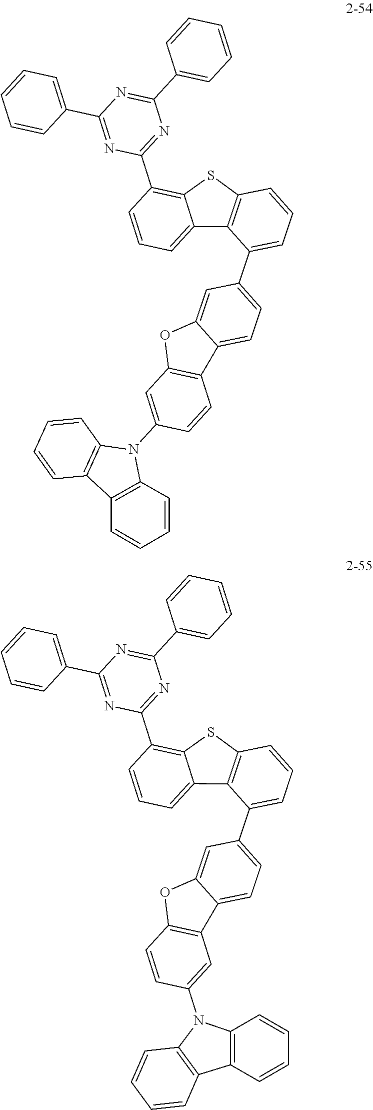 [SMILES: c1ccc(-c2nc(-c3ccccc3)nc(-c3cccc4c3sc3cccc(-c5ccc6c(c5)oc5cc(-n7c8ccccc8c8ccccc87)ccc56)c34)n2)cc1 ; c1ccc(-c2nc(-c3ccccc3)nc(-c3cccc4c3sc3cccc(-c5ccc6c(c5)oc5ccc(-n7c8ccccc8c8ccccc87)cc56)c34)n2)cc1]

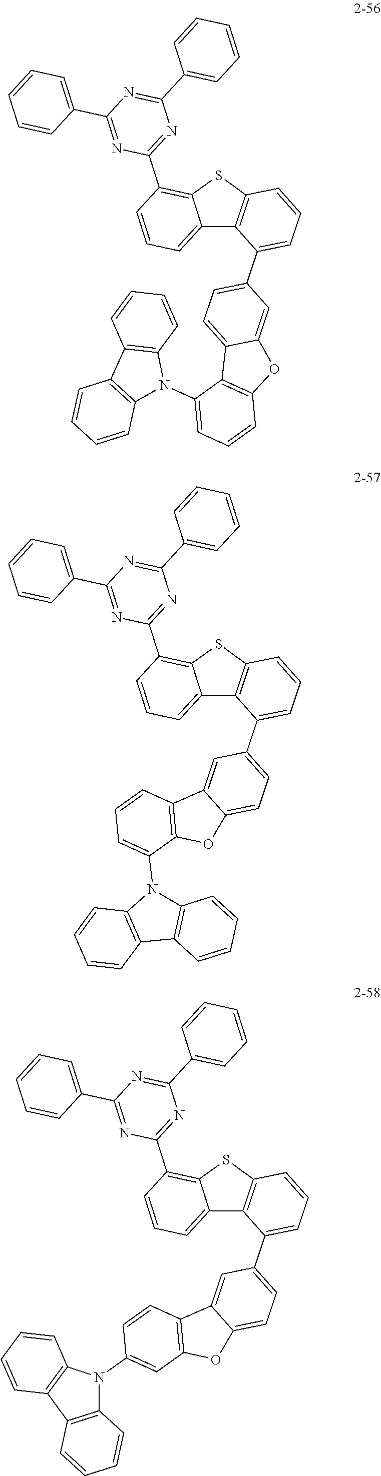

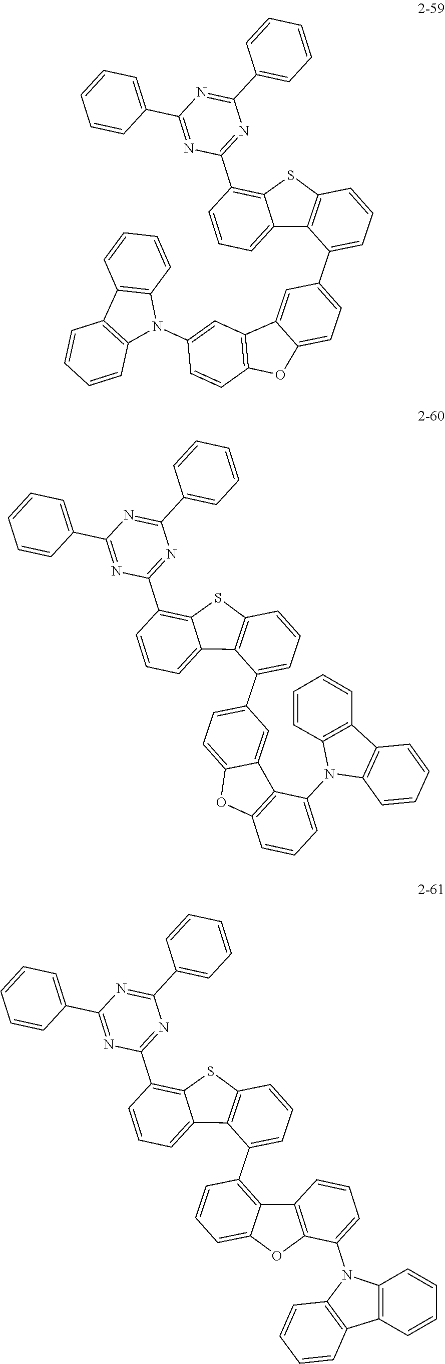 [SMILES: c1ccc(-c2nc(-c3ccccc3)nc(-c3cccc4c3sc3cccc(-c5ccc6oc7ccc(-n8c9ccccc9c9ccccc98)cc7c6c5)c34)n2)cc1 ; c1ccc(-c2nc(-c3ccccc3)nc(-c3cccc4c3sc3cccc(-c5ccc6oc7cccc(-n8c9ccccc9c9ccccc98)c7c6c5)c34)n2)cc1 ; c1ccc(-c2nc(-c3ccccc3)nc(-c3cccc4c3sc3cccc(-c5cccc6oc7c(-n8c9ccccc9c9ccccc98)cccc7c56)c34)n2)cc1]

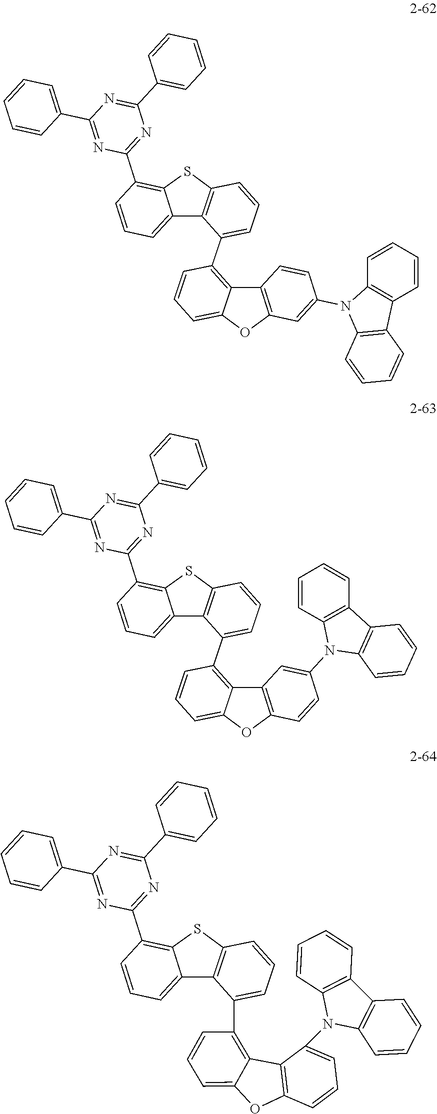 [SMILES: c1ccc(-c2nc(-c3ccccc3)nc(-c3cccc4c3sc3cccc(-c5cccc6oc7cc(-n8c9ccccc9c9ccccc98)ccc7c56)c34)n2)cc1 ; c1ccc(-c2nc(-c3ccccc3)nc(-c3cccc4c3sc3cccc(-c5cccc6oc7ccc(-n8c9ccccc9c9ccccc98)cc7c56)c34)n2)cc1 ; c1ccc(-c2nc(-c3ccccc3)nc(-c3cccc4c3sc3cccc(-c5cccc6oc7cccc(-n8c9ccccc9c9ccccc98)c7c56)c34)n2)cc1]

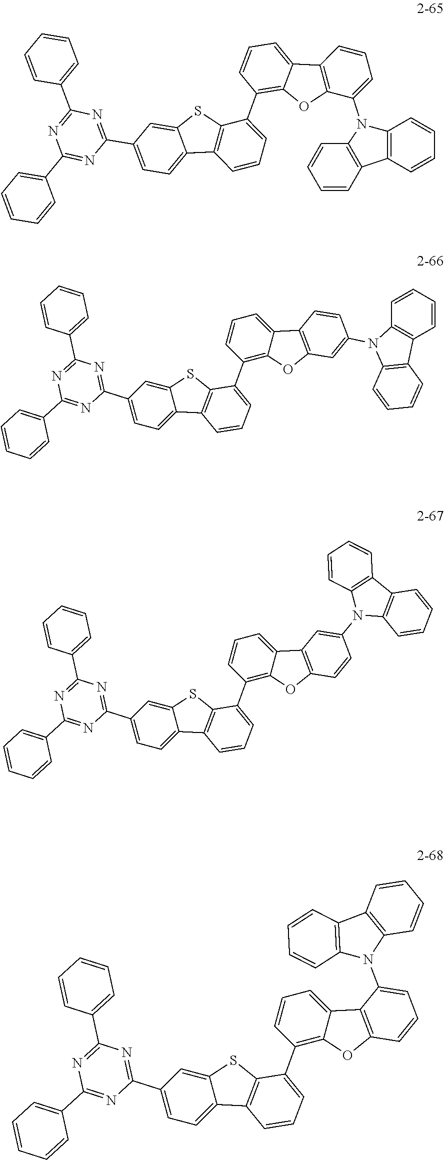

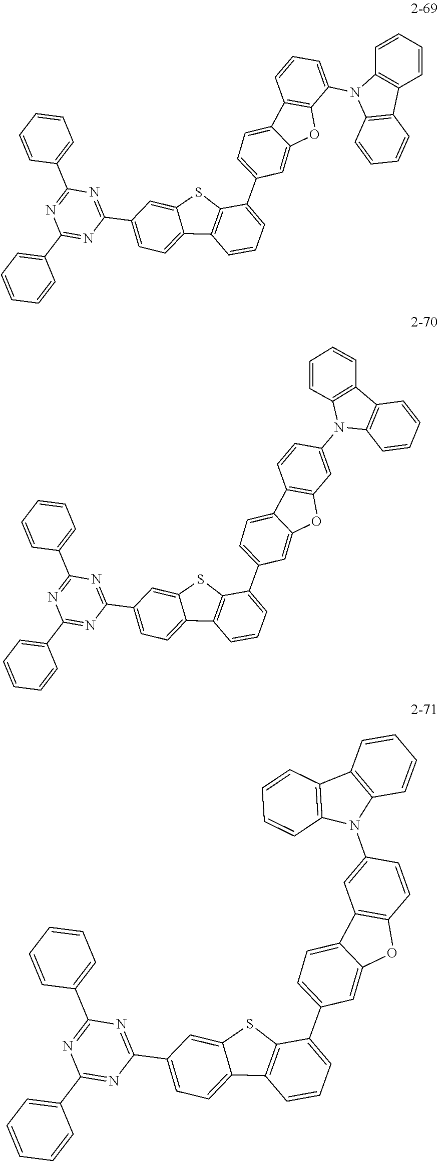 [SMILES: c1ccc(-c2nc(-c3ccccc3)nc(-c3ccc4c(c3)sc3c(-c5ccc6c(c5)oc5c(-n7c8ccccc8c8ccccc87)cccc56)cccc34)n2)cc1 ; c1ccc(-c2nc(-c3ccccc3)nc(-c3ccc4c(c3)sc3c(-c5ccc6c(c5)oc5cc(-n7c8ccccc8c8ccccc87)ccc56)cccc34)n2)cc1 ; c1ccc(-c2nc(-c3ccccc3)nc(-c3ccc4c(c3)sc3c(-c5ccc6c(c5)oc5ccc(-n7c8ccccc8c8ccccc87)cc56)cccc34)n2)cc1]

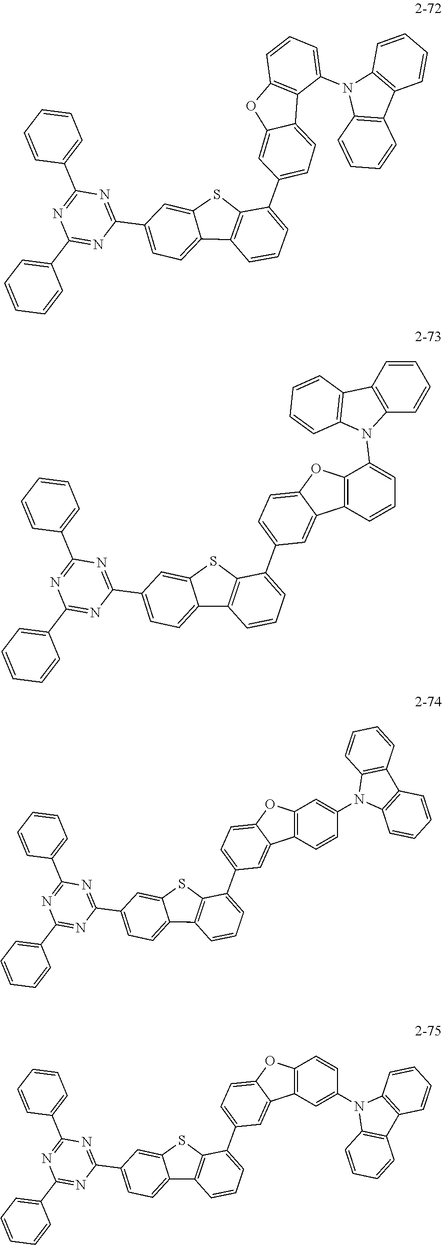 [SMILES: c1ccc(-c2nc(-c3ccccc3)nc(-c3ccc4c(c3)sc3c(-c5ccc6c(c5)oc5cccc(-n7c8ccccc8c8ccccc87)c56)cccc34)n2)cc1 ; c1ccc(-c2nc(-c3ccccc3)nc(-c3ccc4c(c3)sc3c(-c5ccc6oc7c(-n8c9ccccc9c9ccccc98)cccc7c6c5)cccc34)n2)cc1 ; c1ccc(-c2nc(-c3ccccc3)nc(-c3ccc4c(c3)sc3c(-c5ccc6oc7cc(-n8c9ccccc9c9ccccc98)ccc7c6c5)cccc34)n2)cc1 ; c1ccc(-c2nc(-c3ccccc3)nc(-c3ccc4c(c3)sc3c(-c5ccc6oc7ccc(-n8c9ccccc9c9ccccc98)cc7c6c5)cccc34)n2)cc1]

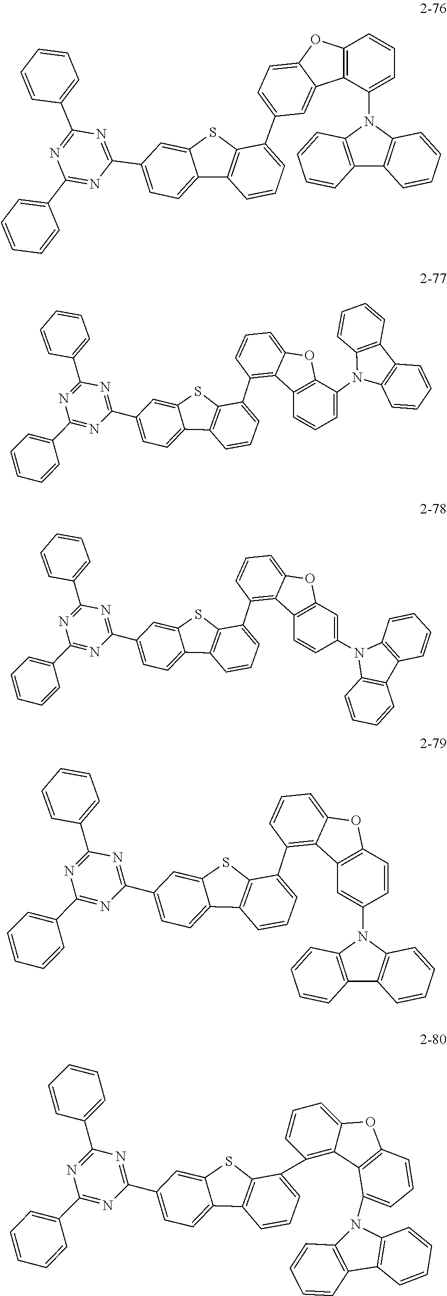

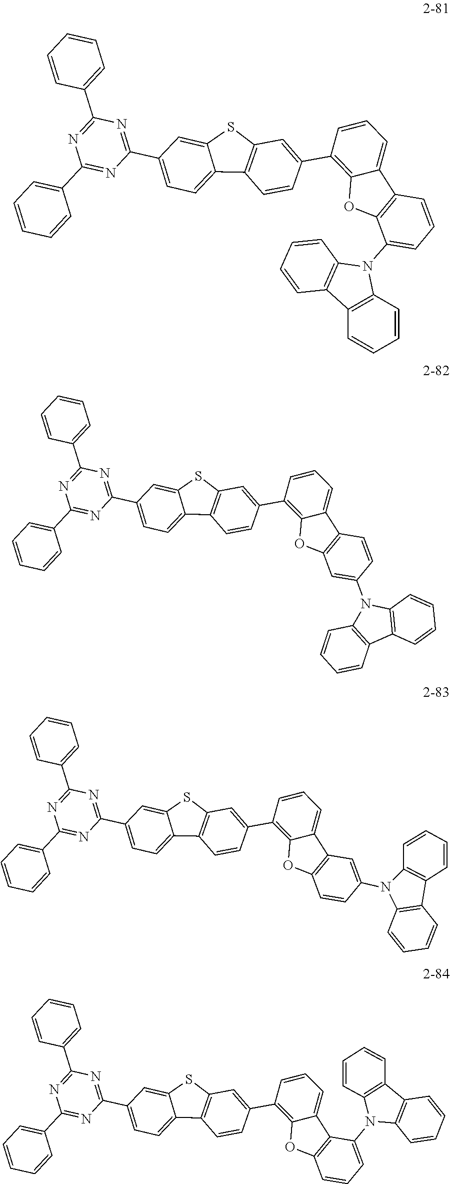 [SMILES: c1ccc(-c2nc(-c3ccccc3)nc(-c3ccc4c(c3)sc3cc(-c5cccc6c5oc5c(-n7c8ccccc8c8ccccc87)cccc56)ccc34)n2)cc1 ; c1ccc(-c2nc(-c3ccccc3)nc(-c3ccc4c(c3)sc3cc(-c5cccc6c5oc5cc(-n7c8ccccc8c8ccccc87)ccc56)ccc34)n2)cc1 ; c1ccc(-c2nc(-c3ccccc3)nc(-c3ccc4c(c3)sc3cc(-c5cccc6c5oc5ccc(-n7c8ccccc8c8ccccc87)cc56)ccc34)n2)cc1 ; c1ccc(-c2nc(-c3ccccc3)nc(-c3ccc4c(c3)sc3cc(-c5cccc6c5oc5cccc(-n7c8ccccc8c8ccccc87)c56)ccc34)n2)cc1]

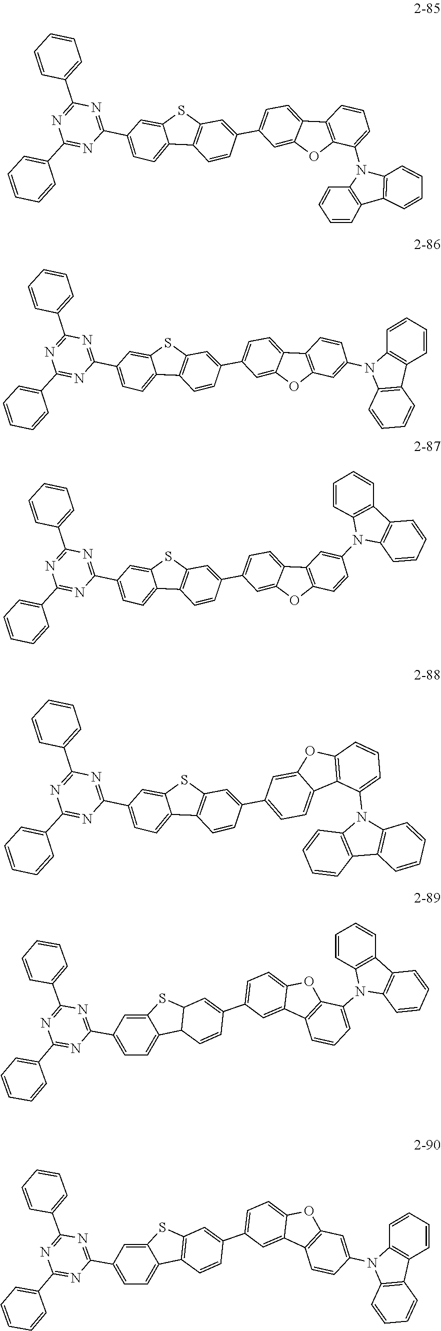

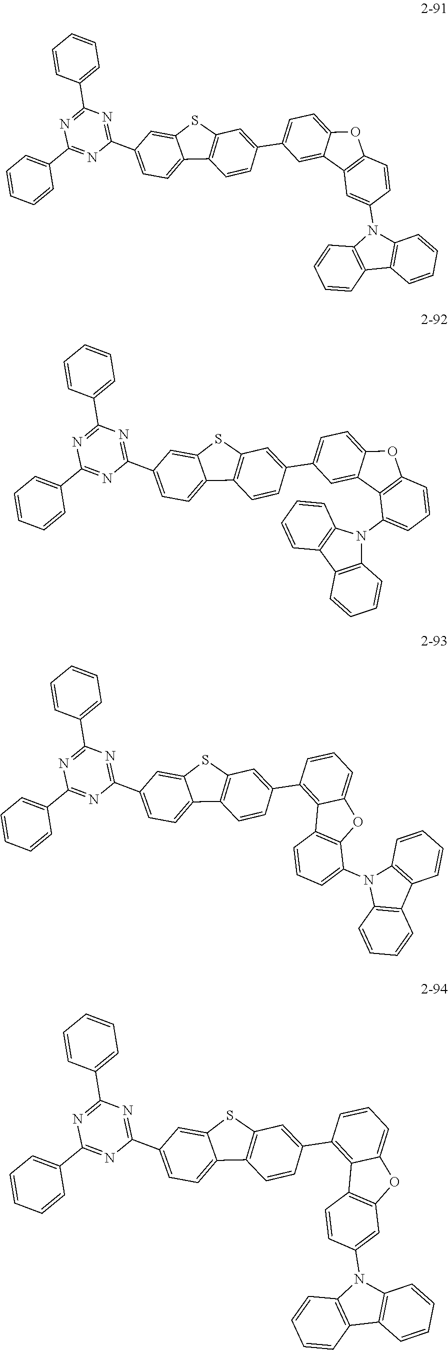 [SMILES: c1ccc(-c2nc(-c3ccccc3)nc(-c3ccc4c(c3)sc3cc(-c5ccc6oc7ccc(-n8c9ccccc9c9ccccc98)cc7c6c5)ccc34)n2)cc1 ; c1ccc(-c2nc(-c3ccccc3)nc(-c3ccc4c(c3)sc3cc(-c5ccc6oc7cccc(-n8c9ccccc9c9ccccc98)c7c6c5)ccc34)n2)cc1 ; c1ccc(-c2nc(-c3ccccc3)nc(-c3ccc4c(c3)sc3cc(-c5cccc6oc7c(-n8c9ccccc9c9ccccc98)cccc7c56)ccc34)n2)cc1 ; c1ccc(-c2nc(-c3ccccc3)nc(-c3ccc4c(c3)sc3cc(-c5cccc6oc7cc(-n8c9ccccc9c9ccccc98)ccc7c56)ccc34)n2)cc1]

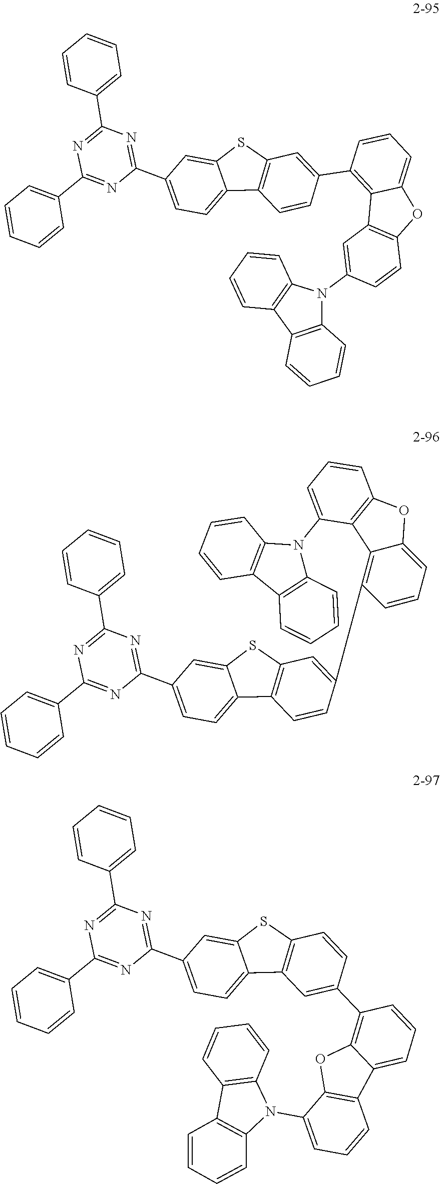 [SMILES: c1ccc(-c2nc(-c3ccccc3)nc(-c3ccc4c(c3)sc3cc(-c5cccc6oc7ccc(-n8c9ccccc9c9ccccc98)cc7c56)ccc34)n2)cc1 ; c1ccc(-c2nc(-c3ccccc3)nc(-c3ccc4c(c3)sc3cc(CCc5cccc6oc7cccc(-n8c9ccccc9c9ccccc98)c7c56)ccc34)n2)cc1 ; c1ccc(-c2nc(-c3ccccc3)nc(-c3ccc4c(c3)sc3ccc(-c5cccc6c5oc5c(-n7c8ccccc8c8ccccc87)cccc56)cc34)n2)cc1]

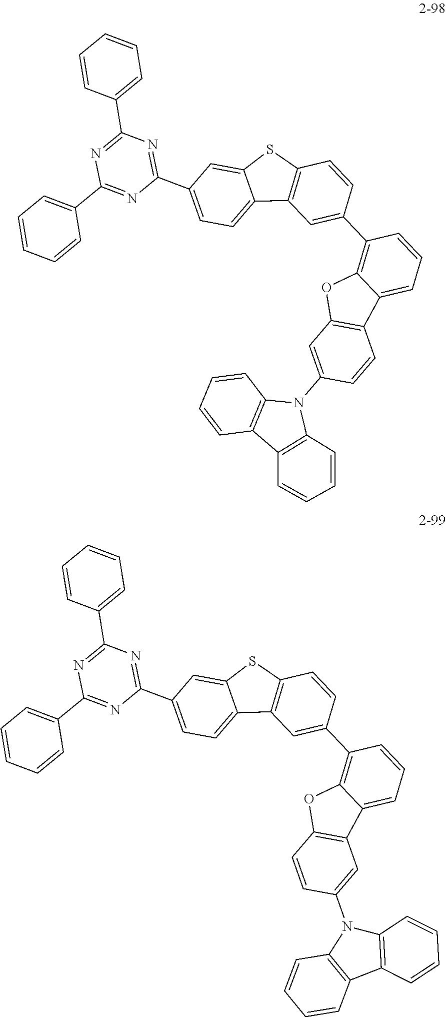 [SMILES: c1ccc(-c2nc(-c3ccccc3)nc(-c3ccc4c(c3)sc3ccc(-c5cccc6c5oc5cc(-n7c8ccccc8c8ccccc87)ccc56)cc34)n2)cc1 ; c1ccc(-c2nc(-c3ccccc3)nc(-c3ccc4c(c3)sc3ccc(-c5cccc6c5oc5ccc(-n7c8ccccc8c8ccccc87)cc56)cc34)n2)cc1]

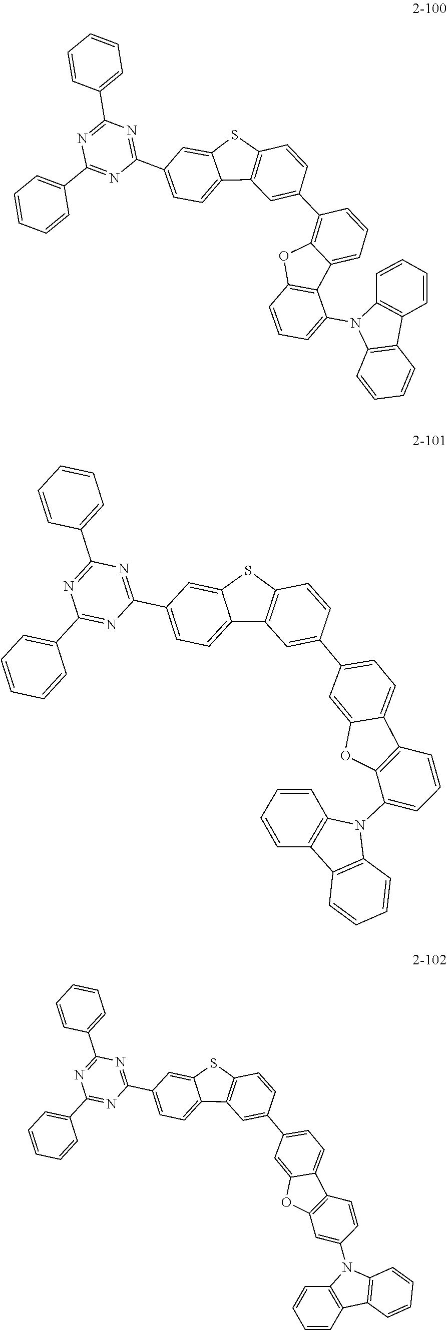 [SMILES: c1ccc(-c2nc(-c3ccccc3)nc(-c3ccc4c(c3)sc3ccc(-c5cccc6c5oc5cccc(-n7c8ccccc8c8ccccc87)c56)cc34)n2)cc1 ; c1ccc(-c2nc(-c3ccccc3)nc(-c3ccc4c(c3)sc3ccc(-c5ccc6c(c5)oc5c(-n7c8ccccc8c8ccccc87)cccc56)cc34)n2)cc1 ; c1ccc(-c2nc(-c3ccccc3)nc(-c3ccc4c(c3)sc3ccc(-c5ccc6c(c5)oc5cc(-n7c8ccccc8c8ccccc87)ccc56)cc34)n2)cc1]

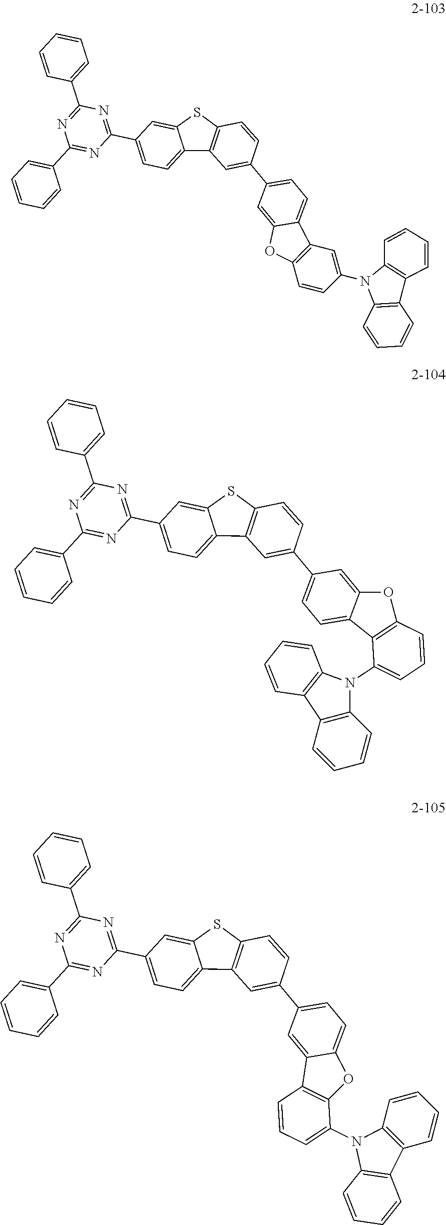 [SMILES: c1ccc(-c2nc(-c3ccccc3)nc(-c3ccc4c(c3)sc3ccc(-c5ccc6c(c5)oc5ccc(-n7c8ccccc8c8ccccc87)cc56)cc34)n2)cc1 ; c1ccc(-c2nc(-c3ccccc3)nc(-c3ccc4c(c3)sc3ccc(-c5ccc6c(c5)oc5cccc(-n7c8ccccc8c8ccccc87)c56)cc34)n2)cc1 ; c1ccc(-c2nc(-c3ccccc3)nc(-c3ccc4c(c3)sc3ccc(-c5ccc6oc7c(-n8c9ccccc9c9ccccc98)cccc7c6c5)cc34)n2)cc1]

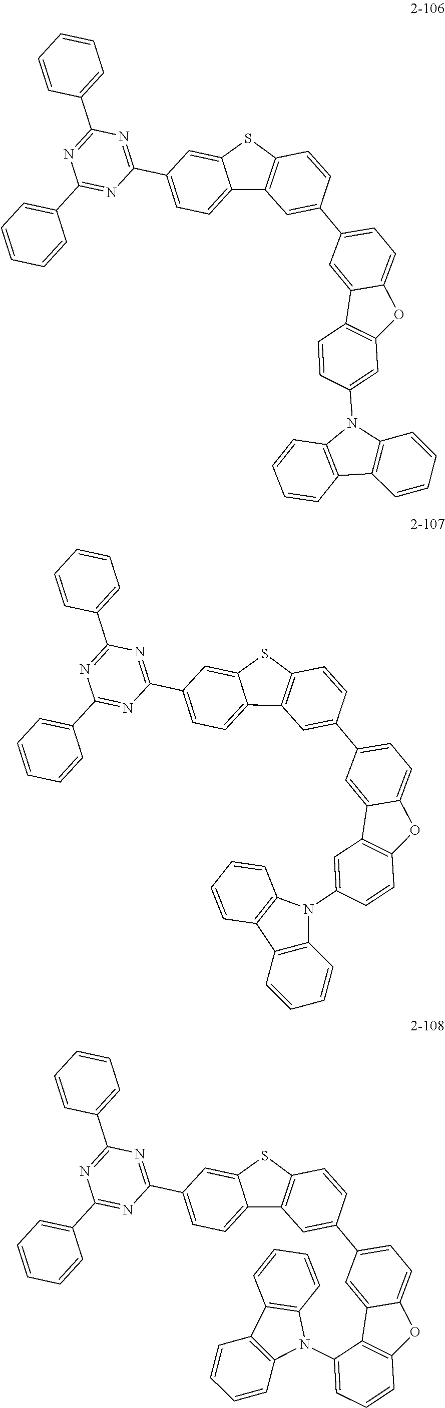 [SMILES: c1ccc(-c2nc(-c3ccccc3)nc(-c3ccc4c(c3)sc3ccc(-c5ccc6oc7cc(-n8c9ccccc9c9ccccc98)ccc7c6c5)cc34)n2)cc1 ; c1ccc(-c2nc(-c3ccccc3)nc(-c3ccc4c(c3)sc3ccc(-c5ccc6oc7ccc(-n8c9ccccc9c9ccccc98)cc7c6c5)cc34)n2)cc1 ; c1ccc(-c2nc(-c3ccccc3)nc(-c3ccc4c(c3)sc3ccc(-c5ccc6oc7cccc(-n8c9ccccc9c9ccccc98)c7c6c5)cc34)n2)cc1]

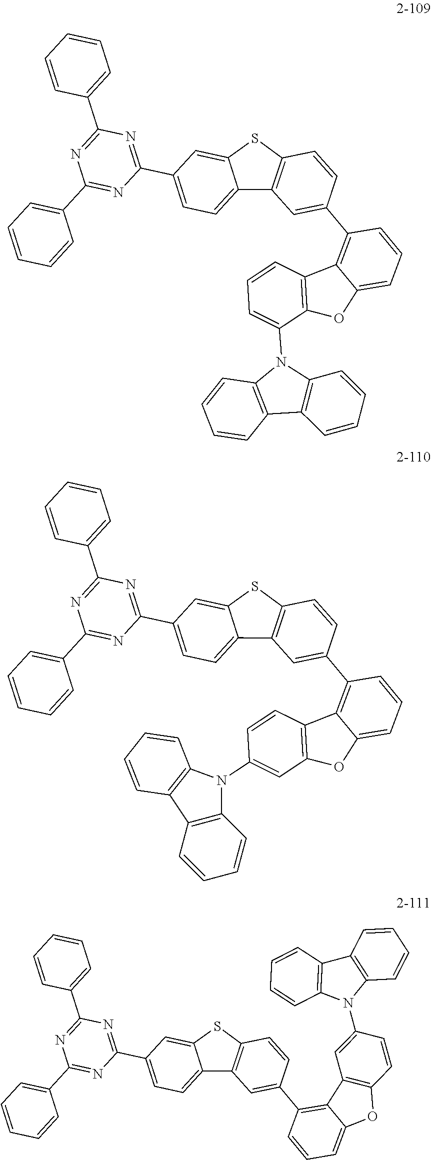 [SMILES: c1ccc(-c2nc(-c3ccccc3)nc(-c3ccc4c(c3)sc3ccc(-c5cccc6oc7c(-n8c9ccccc9c9ccccc98)cccc7c56)cc34)n2)cc1 ; c1ccc(-c2nc(-c3ccccc3)nc(-c3ccc4c(c3)sc3ccc(-c5cccc6oc7cc(-n8c9ccccc9c9ccccc98)ccc7c56)cc34)n2)cc1 ; c1ccc(-c2nc(-c3ccccc3)nc(-c3ccc4c(c3)sc3ccc(-c5cccc6oc7ccc(-n8c9ccccc9c9ccccc98)cc7c56)cc34)n2)cc1]

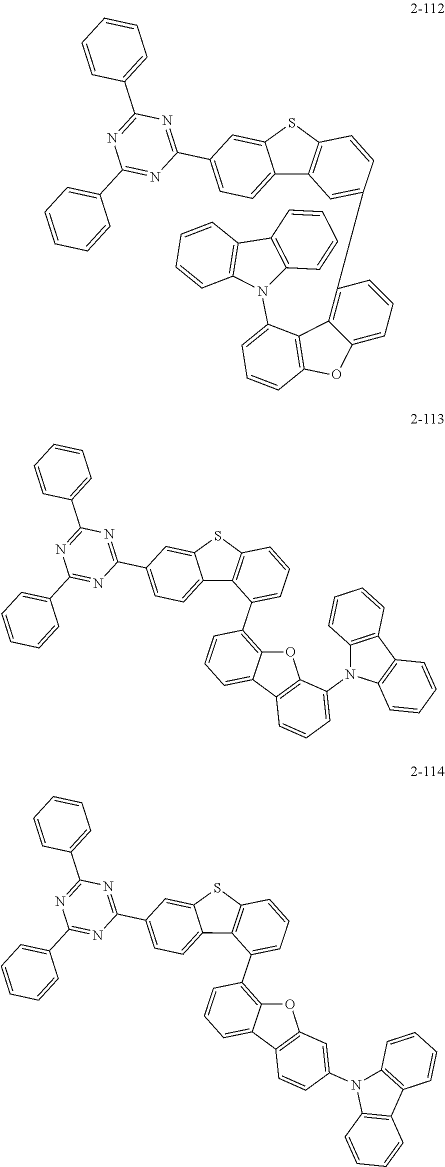 [SMILES: c1ccc(-c2nc(-c3ccccc3)nc(-c3ccc4c(c3)sc3ccc(CCc5cccc6oc7cccc(-n8c9ccccc9c9ccccc98)c7c56)cc34)n2)cc1 ; c1ccc(-c2nc(-c3ccccc3)nc(-c3ccc4c(c3)sc3cccc(-c5cccc6c5oc5c(-n7c8ccccc8c8ccccc87)cccc56)c34)n2)cc1 ; c1ccc(-c2nc(-c3ccccc3)nc(-c3ccc4c(c3)sc3cccc(-c5cccc6c5oc5cc(-n7c8ccccc8c8ccccc87)ccc56)c34)n2)cc1]

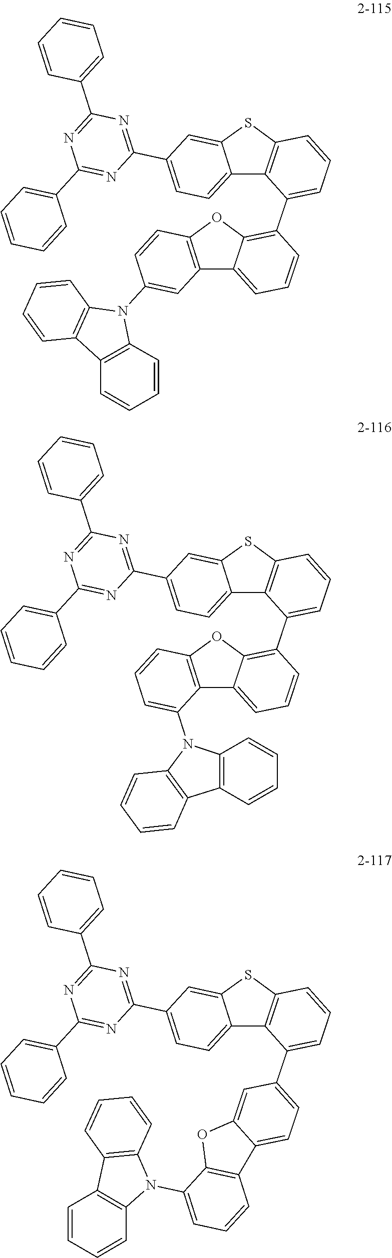 [SMILES: c1ccc(-c2nc(-c3ccccc3)nc(-c3ccc4c(c3)sc3cccc(-c5cccc6c5oc5ccc(-n7c8ccccc8c8ccccc87)cc56)c34)n2)cc1 ; c1ccc(-c2nc(-c3ccccc3)nc(-c3ccc4c(c3)sc3cccc(-c5cccc6c5oc5cccc(-n7c8ccccc8c8ccccc87)c56)c34)n2)cc1 ; c1ccc(-c2nc(-c3ccccc3)nc(-c3ccc4c(c3)sc3cccc(-c5ccc6c(c5)oc5c(-n7c8ccccc8c8ccccc87)cccc56)c34)n2)cc1]

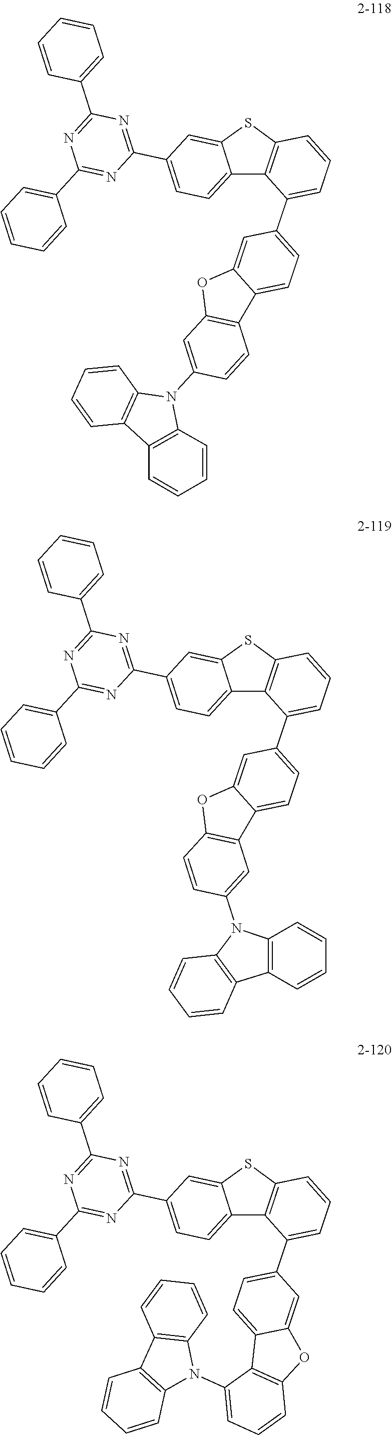 [SMILES: c1ccc(-c2nc(-c3ccccc3)nc(-c3ccc4c(c3)sc3cccc(-c5ccc6c(c5)oc5cc(-n7c8ccccc8c8ccccc87)ccc56)c34)n2)cc1 ; c1ccc(-c2nc(-c3ccccc3)nc(-c3ccc4c(c3)sc3cccc(-c5ccc6c(c5)oc5ccc(-n7c8ccccc8c8ccccc87)cc56)c34)n2)cc1 ; c1ccc(-c2nc(-c3ccccc3)nc(-c3ccc4c(c3)sc3cccc(-c5ccc6c(c5)oc5cccc(-n7c8ccccc8c8ccccc87)c56)c34)n2)cc1]

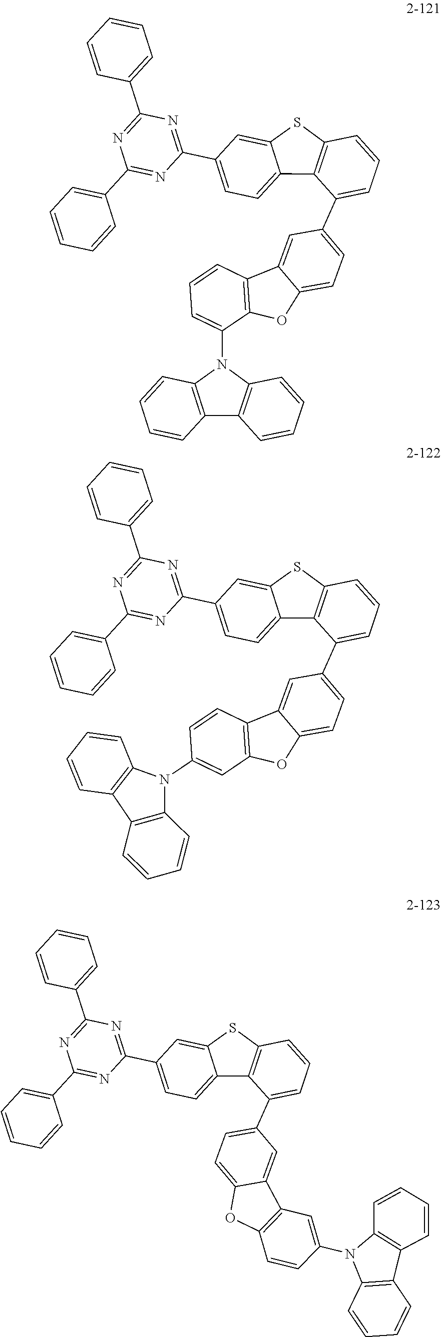

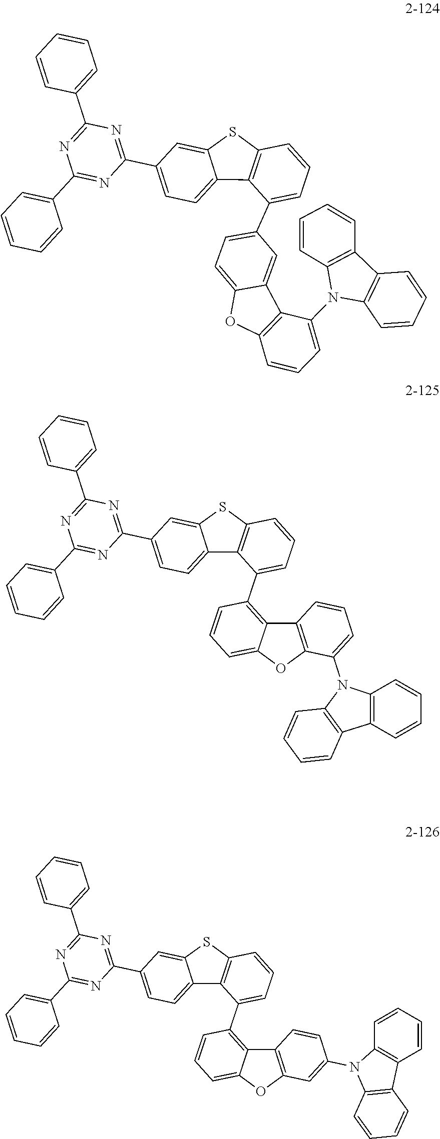 [SMILES: c1ccc(-c2nc(-c3ccccc3)nc(-c3ccc4c(c3)sc3cccc(-c5ccc6oc7cccc(-n8c9ccccc9c9ccccc98)c7c6c5)c34)n2)cc1 ; c1ccc(-c2nc(-c3ccccc3)nc(-c3ccc4c(c3)sc3cccc(-c5cccc6oc7c(-n8c9ccccc9c9ccccc98)cccc7c56)c34)n2)cc1 ; c1ccc(-c2nc(-c3ccccc3)nc(-c3ccc4c(c3)sc3cccc(-c5cccc6oc7cc(-n8c9ccccc9c9ccccc98)ccc7c56)c34)n2)cc1]

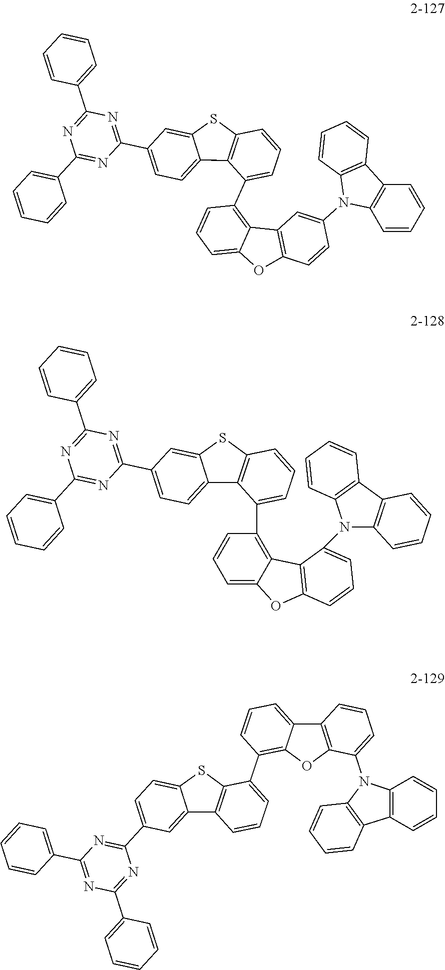 [SMILES: c1ccc(-c2nc(-c3ccccc3)nc(-c3ccc4c(c3)sc3cccc(-c5cccc6oc7ccc(-n8c9ccccc9c9ccccc98)cc7c56)c34)n2)cc1 ; c1ccc(-c2nc(-c3ccccc3)nc(-c3ccc4c(c3)sc3cccc(-c5cccc6oc7cccc(-n8c9ccccc9c9ccccc98)c7c56)c34)n2)cc1 ; c1ccc(-c2nc(-c3ccccc3)nc(-c3ccc4sc5c(-c6cccc7c6oc6c(-n8c9ccccc9c9ccccc98)cccc67)cccc5c4c3)n2)cc1]

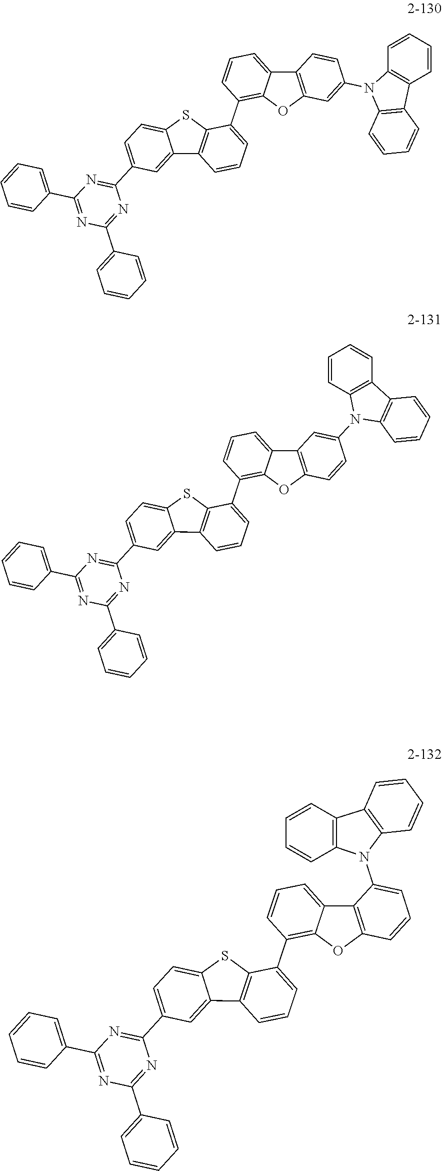 [SMILES: c1ccc(-c2nc(-c3ccccc3)nc(-c3ccc4sc5c(-c6cccc7c6oc6cc(-n8c9ccccc9c9ccccc98)ccc67)cccc5c4c3)n2)cc1 ; c1ccc(-c2nc(-c3ccccc3)nc(-c3ccc4sc5c(-c6cccc7c6oc6ccc(-n8c9ccccc9c9ccccc98)cc67)cccc5c4c3)n2)cc1 ; c1ccc(-c2nc(-c3ccccc3)nc(-c3ccc4sc5c(-c6cccc7c6oc6cccc(-n8c9ccccc9c9ccccc98)c67)cccc5c4c3)n2)cc1]

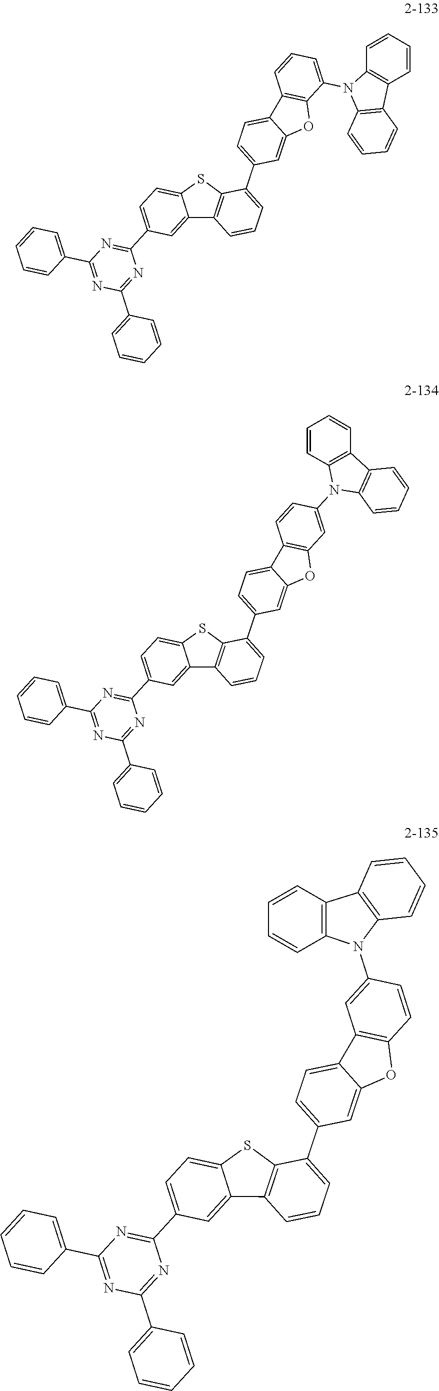 [SMILES: c1ccc(-c2nc(-c3ccccc3)nc(-c3ccc4sc5c(-c6ccc7c(c6)oc6c(-n8c9ccccc9c9ccccc98)cccc67)cccc5c4c3)n2)cc1 ; c1ccc(-c2nc(-c3ccccc3)nc(-c3ccc4sc5c(-c6ccc7c(c6)oc6cc(-n8c9ccccc9c9ccccc98)ccc67)cccc5c4c3)n2)cc1 ; c1ccc(-c2nc(-c3ccccc3)nc(-c3ccc4sc5c(-c6ccc7c(c6)oc6ccc(-n8c9ccccc9c9ccccc98)cc67)cccc5c4c3)n2)cc1]

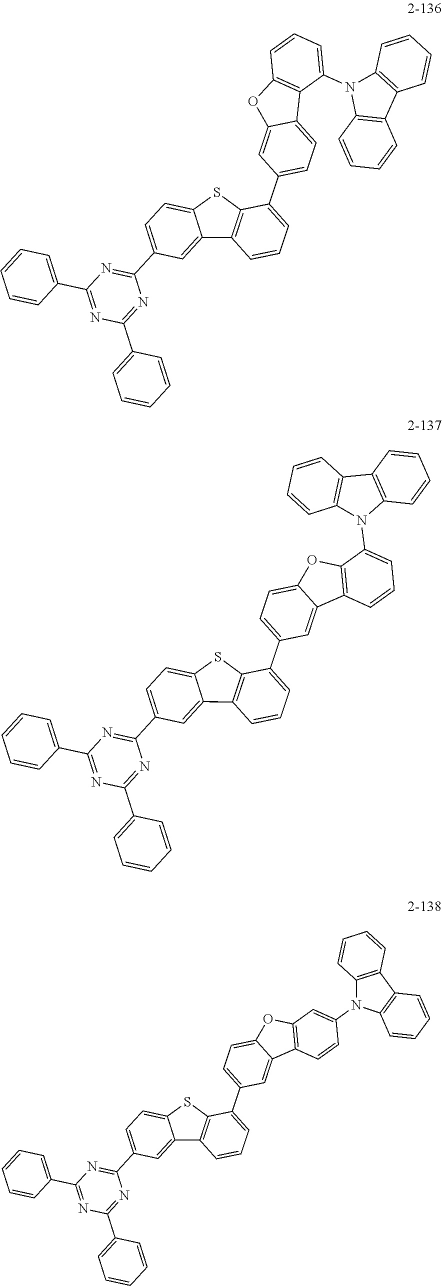 [SMILES: c1ccc(-c2nc(-c3ccccc3)nc(-c3ccc4sc5c(-c6ccc7c(c6)oc6cccc(-n8c9ccccc9c9ccccc98)c67)cccc5c4c3)n2)cc1 ; c1ccc(-c2nc(-c3ccccc3)nc(-c3ccc4sc5c(-c6ccc7oc8c(-n9c%10ccccc%10c%10ccccc%109)cccc8c7c6)cccc5c4c3)n2)cc1 ; c1ccc(-c2nc(-c3ccccc3)nc(-c3ccc4sc5c(-c6ccc7oc8cc(-n9c%10ccccc%10c%10ccccc%109)ccc8c7c6)cccc5c4c3)n2)cc1]

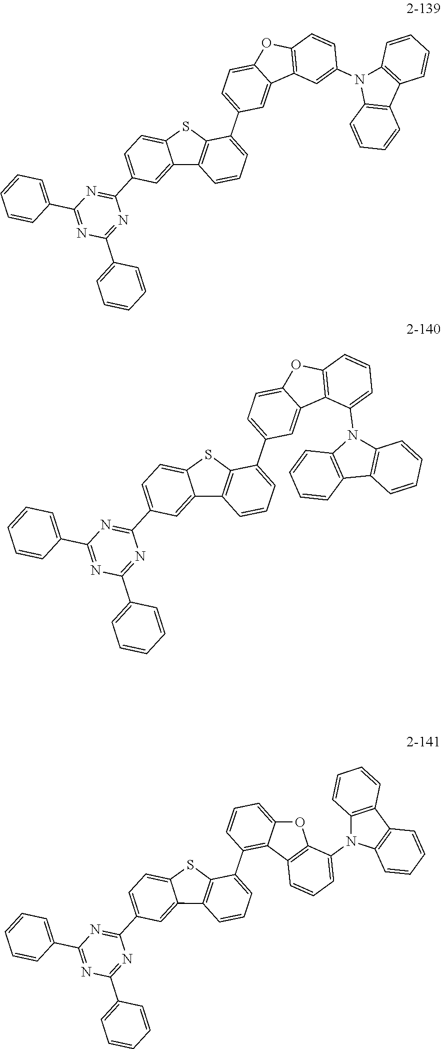 [SMILES: c1ccc(-c2nc(-c3ccccc3)nc(-c3ccc4sc5c(-c6ccc7oc8ccc(-n9c%10ccccc%10c%10ccccc%109)cc8c7c6)cccc5c4c3)n2)cc1 ; c1ccc(-c2nc(-c3ccccc3)nc(-c3ccc4sc5c(-c6ccc7oc8cccc(-n9c%10ccccc%10c%10ccccc%109)c8c7c6)cccc5c4c3)n2)cc1 ; c1ccc(-c2nc(-c3ccccc3)nc(-c3ccc4sc5c(-c6cccc7oc8c(-n9c%10ccccc%10c%10ccccc%109)cccc8c67)cccc5c4c3)n2)cc1]

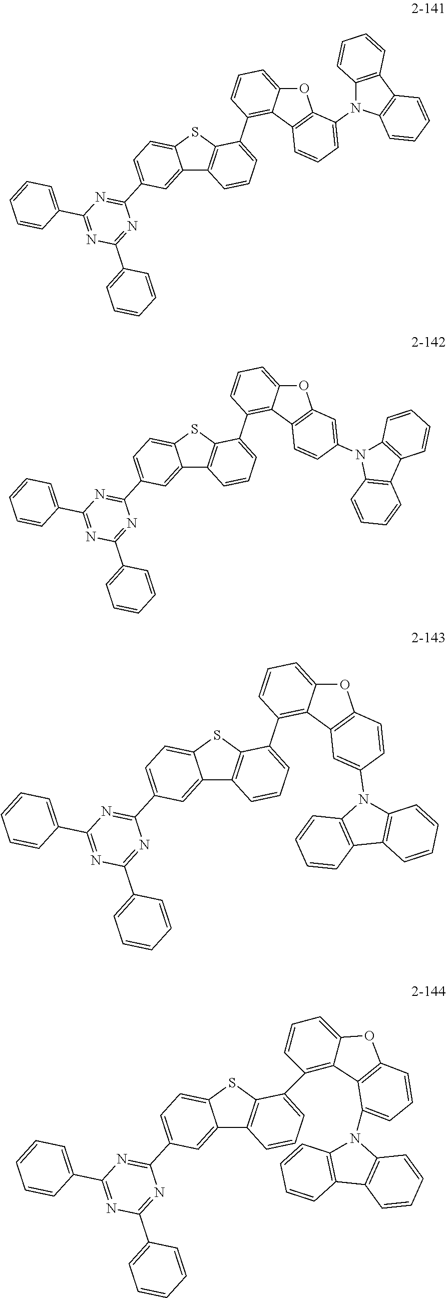 [SMILES: c1ccc(-c2nc(-c3ccccc3)nc(-c3ccc4sc5c(-c6cccc7oc8c(-n9c%10ccccc%10c%10ccccc%109)cccc8c67)cccc5c4c3)n2)cc1 ; c1ccc(-c2nc(-c3ccccc3)nc(-c3ccc4sc5c(-c6cccc7oc8cc(-n9c%10ccccc%10c%10ccccc%109)ccc8c67)cccc5c4c3)n2)cc1 ; c1ccc(-c2nc(-c3ccccc3)nc(-c3ccc4sc5c(-c6cccc7oc8ccc(-n9c%10ccccc%10c%10ccccc%109)cc8c67)cccc5c4c3)n2)cc1 ; c1ccc(-c2nc(-c3ccccc3)nc(-c3ccc4sc5c(-c6cccc7oc8cccc(-n9c%10ccccc%10c%10ccccc%109)c8c67)cccc5c4c3)n2)cc1]

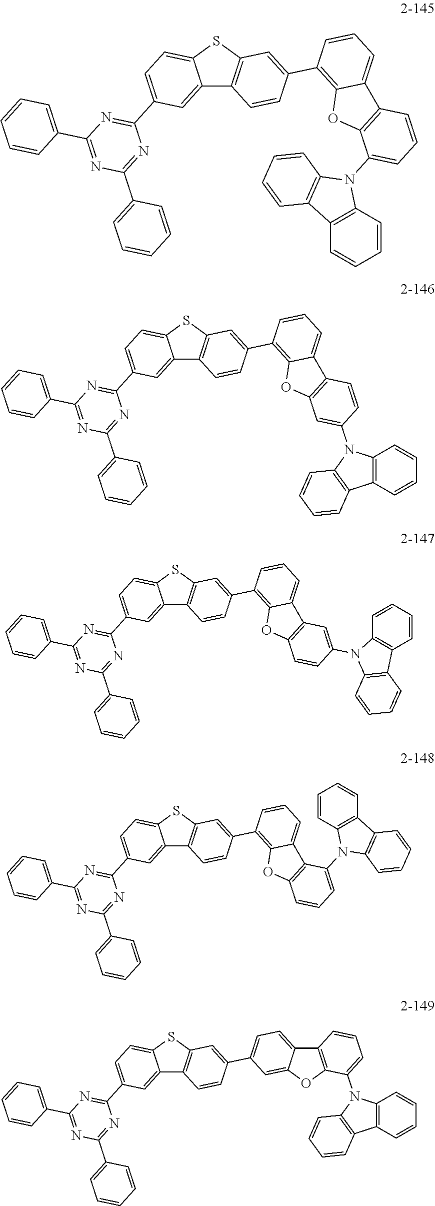 [SMILES: c1ccc(-c2nc(-c3ccccc3)nc(-c3ccc4sc5cc(-c6cccc7c6oc6c(-n8c9ccccc9c9ccccc98)cccc67)ccc5c4c3)n2)cc1 ; c1ccc(-c2nc(-c3ccccc3)nc(-c3ccc4sc5cc(-c6cccc7c6oc6cc(-n8c9ccccc9c9ccccc98)ccc67)ccc5c4c3)n2)cc1 ; c1ccc(-c2nc(-c3ccccc3)nc(-c3ccc4sc5cc(-c6cccc7c6oc6ccc(-n8c9ccccc9c9ccccc98)cc67)ccc5c4c3)n2)cc1 ; c1ccc(-c2nc(-c3ccccc3)nc(-c3ccc4sc5cc(-c6cccc7c6oc6cccc(-n8c9ccccc9c9ccccc98)c67)ccc5c4c3)n2)cc1 ; c1ccc(-c2nc(-c3ccccc3)nc(-c3ccc4sc5cc(-c6ccc7c(c6)oc6c(-n8c9ccccc9c9ccccc98)cccc67)ccc5c4c3)n2)cc1]

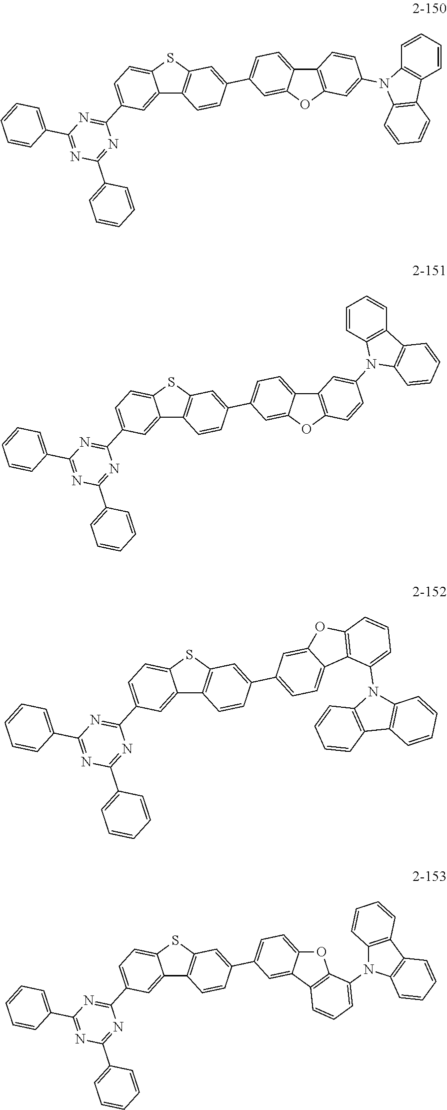 [SMILES: c1ccc(-c2nc(-c3ccccc3)nc(-c3ccc4sc5cc(-c6ccc7c(c6)oc6cc(-n8c9ccccc9c9ccccc98)ccc67)ccc5c4c3)n2)cc1 ; c1ccc(-c2nc(-c3ccccc3)nc(-c3ccc4sc5cc(-c6ccc7c(c6)oc6ccc(-n8c9ccccc9c9ccccc98)cc67)ccc5c4c3)n2)cc1 ; c1ccc(-c2nc(-c3ccccc3)nc(-c3ccc4sc5cc(-c6ccc7c(c6)oc6cccc(-n8c9ccccc9c9ccccc98)c67)ccc5c4c3)n2)cc1 ; c1ccc(-c2nc(-c3ccccc3)nc(-c3ccc4sc5cc(-c6ccc7oc8c(-n9c%10ccccc%10c%10ccccc%109)cccc8c7c6)ccc5c4c3)n2)cc1]

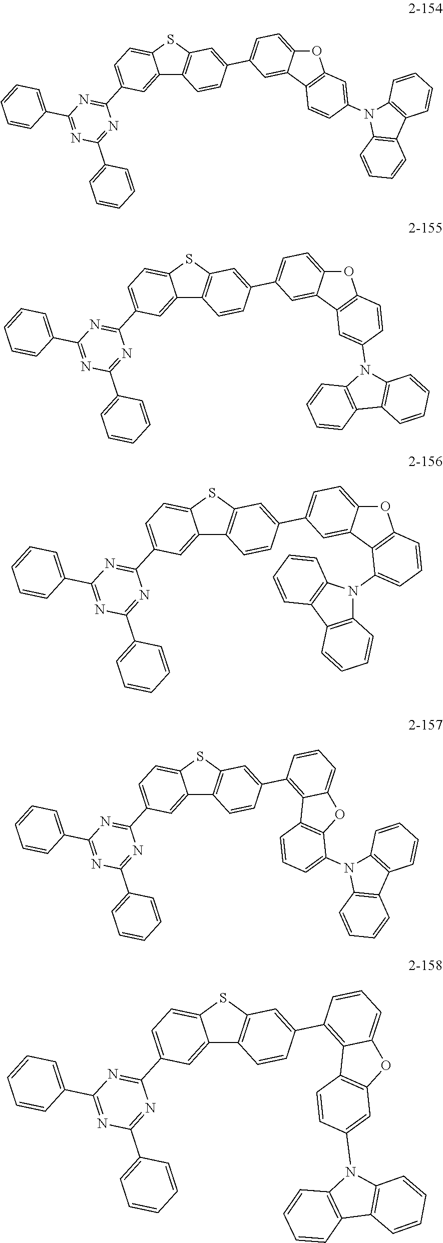

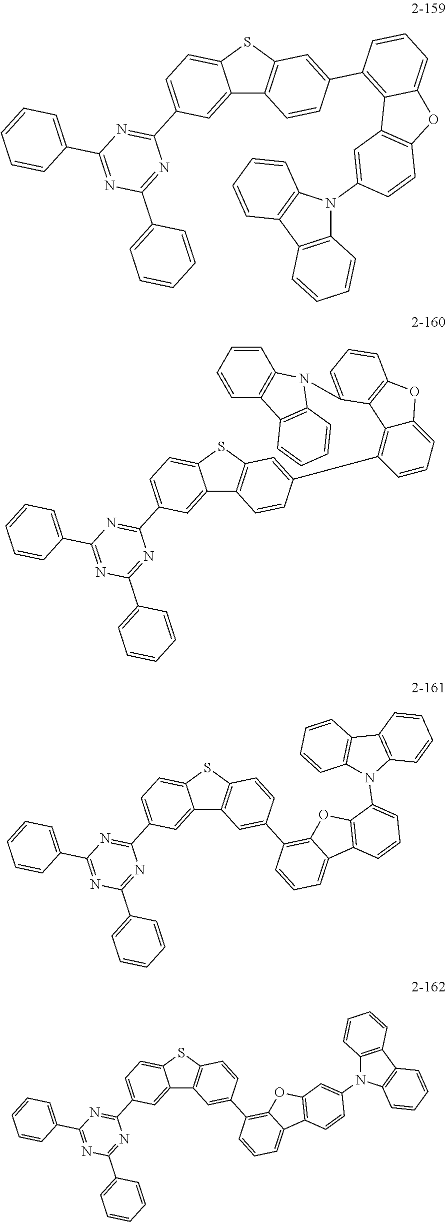 [SMILES: c1ccc(-c2nc(-c3ccccc3)nc(-c3ccc4sc5cc(-c6cccc7oc8ccc(-n9c%10ccccc%10c%10ccccc%109)cc8c67)ccc5c4c3)n2)cc1 ; c1ccc(-c2nc(-c3ccccc3)nc(-c3ccc4sc5cc(CCc6cccc7oc8cccc(-n9c%10ccccc%10c%10ccccc%109)c8c67)ccc5c4c3)n2)cc1 ; c1ccc(-c2nc(-c3ccccc3)nc(-c3ccc4sc5ccc(-c6cccc7c6oc6c(-n8c9ccccc9c9ccccc98)cccc67)cc5c4c3)n2)cc1 ; c1ccc(-c2nc(-c3ccccc3)nc(-c3ccc4sc5ccc(-c6cccc7c6oc6cc(-n8c9ccccc9c9ccccc98)ccc67)cc5c4c3)n2)cc1]

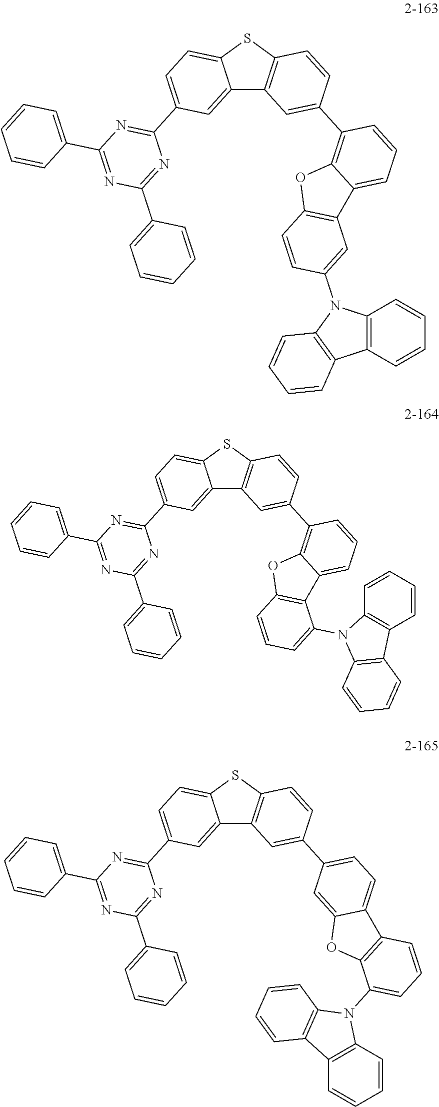 [SMILES: c1ccc(-c2nc(-c3ccccc3)nc(-c3ccc4sc5ccc(-c6cccc7c6oc6ccc(-n8c9ccccc9c9ccccc98)cc67)cc5c4c3)n2)cc1 ; c1ccc(-c2nc(-c3ccccc3)nc(-c3ccc4sc5ccc(-c6cccc7c6oc6cccc(-n8c9ccccc9c9ccccc98)c67)cc5c4c3)n2)cc1 ; c1ccc(-c2nc(-c3ccccc3)nc(-c3ccc4sc5ccc(-c6ccc7c(c6)oc6c(-n8c9ccccc9c9ccccc98)cccc67)cc5c4c3)n2)cc1]

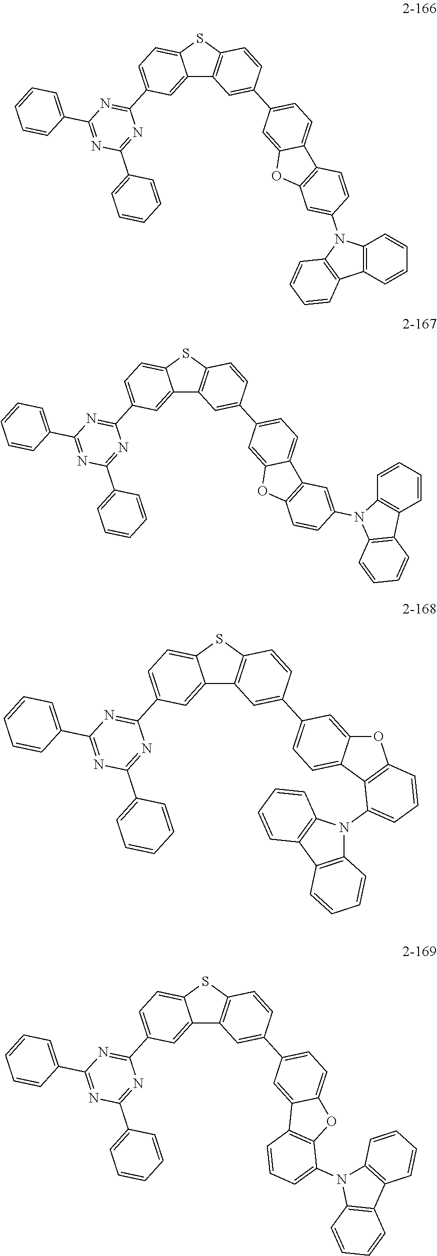 [SMILES: c1ccc(-c2nc(-c3ccccc3)nc(-c3ccc4sc5ccc(-c6ccc7c(c6)oc6cc(-n8c9ccccc9c9ccccc98)ccc67)cc5c4c3)n2)cc1 ; c1ccc(-c2nc(-c3ccccc3)nc(-c3ccc4sc5ccc(-c6ccc7c(c6)oc6ccc(-n8c9ccccc9c9ccccc98)cc67)cc5c4c3)n2)cc1 ; c1ccc(-c2nc(-c3ccccc3)nc(-c3ccc4sc5ccc(-c6ccc7c(c6)oc6cccc(-n8c9ccccc9c9ccccc98)c67)cc5c4c3)n2)cc1 ; c1ccc(-c2nc(-c3ccccc3)nc(-c3ccc4sc5ccc(-c6ccc7oc8c(-n9c%10ccccc%10c%10ccccc%109)cccc8c7c6)cc5c4c3)n2)cc1]

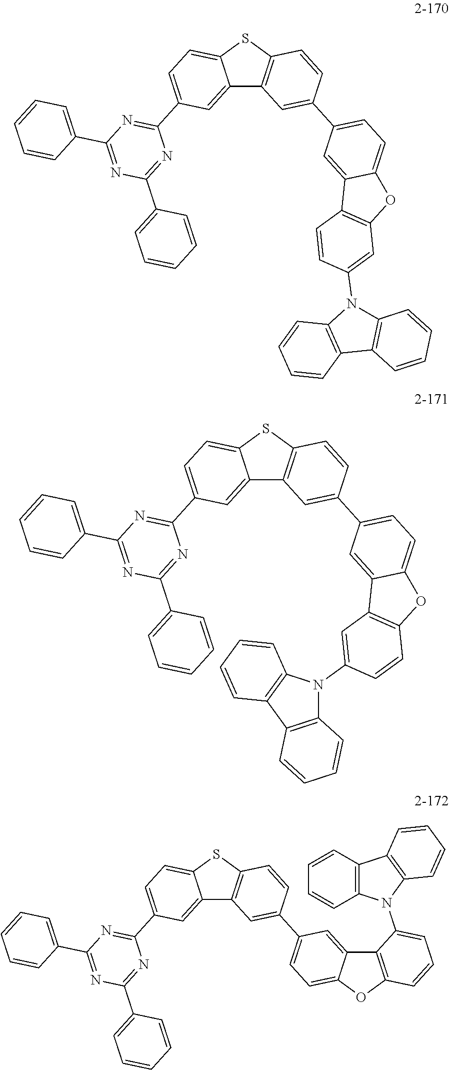 [SMILES: c1ccc(-c2nc(-c3ccccc3)nc(-c3ccc4sc5ccc(-c6ccc7oc8cc(-n9c%10ccccc%10c%10ccccc%109)ccc8c7c6)cc5c4c3)n2)cc1 ; c1ccc(-c2nc(-c3ccccc3)nc(-c3ccc4sc5ccc(-c6ccc7oc8ccc(-n9c%10ccccc%10c%10ccccc%109)cc8c7c6)cc5c4c3)n2)cc1 ; c1ccc(-c2nc(-c3ccccc3)nc(-c3ccc4sc5ccc(-c6ccc7oc8cccc(-n9c%10ccccc%10c%10ccccc%109)c8c7c6)cc5c4c3)n2)cc1]

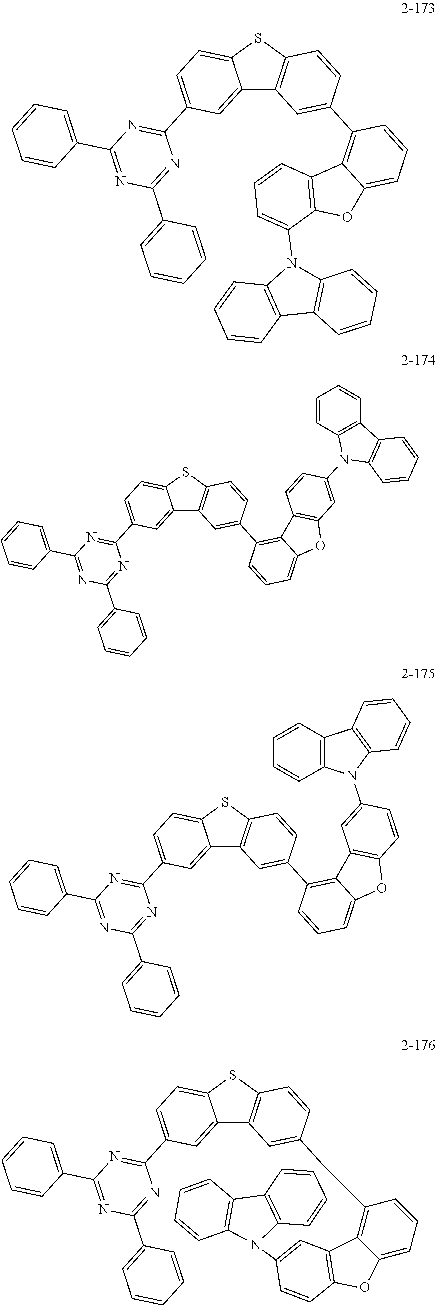 [SMILES: c1ccc(-c2nc(-c3ccccc3)nc(-c3ccc4sc5ccc(-c6cccc7oc8c(-n9c%10ccccc%10c%10ccccc%109)cccc8c67)cc5c4c3)n2)cc1 ; c1ccc(-c2nc(-c3ccccc3)nc(-c3ccc4sc5ccc(-c6cccc7oc8cc(-n9c%10ccccc%10c%10ccccc%109)ccc8c67)cc5c4c3)n2)cc1 ; c1ccc(-c2nc(-c3ccccc3)nc(-c3ccc4sc5ccc(-c6cccc7oc8ccc(-n9c%10ccccc%10c%10ccccc%109)cc8c67)cc5c4c3)n2)cc1 ; c1ccc(-c2nc(-c3ccccc3)nc(-c3ccc4sc5ccc(CCc6cccc7oc8ccc(-n9c%10ccccc%10c%10ccccc%109)cc8c67)cc5c4c3)n2)cc1]

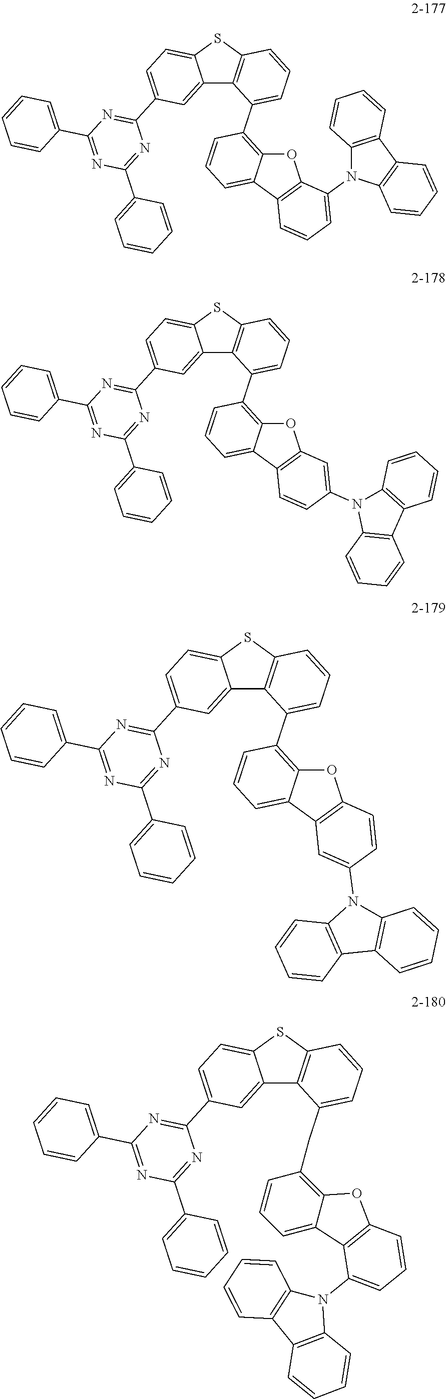 [SMILES: c1ccc(-c2nc(-c3ccccc3)nc(-c3ccc4sc5cccc(-c6cccc7c6oc6c(-n8c9ccccc9c9ccccc98)cccc67)c5c4c3)n2)cc1 ; c1ccc(-c2nc(-c3ccccc3)nc(-c3ccc4sc5cccc(-c6cccc7c6oc6cc(-n8c9ccccc9c9ccccc98)ccc67)c5c4c3)n2)cc1 ; c1ccc(-c2nc(-c3ccccc3)nc(-c3ccc4sc5cccc(-c6cccc7c6oc6ccc(-n8c9ccccc9c9ccccc98)cc67)c5c4c3)n2)cc1 ; c1ccc(-c2nc(-c3ccccc3)nc(-c3ccc4sc5cccc(Cc6cccc7c6oc6cccc(-n8c9ccccc9c9ccccc98)c67)c5c4c3)n2)cc1]

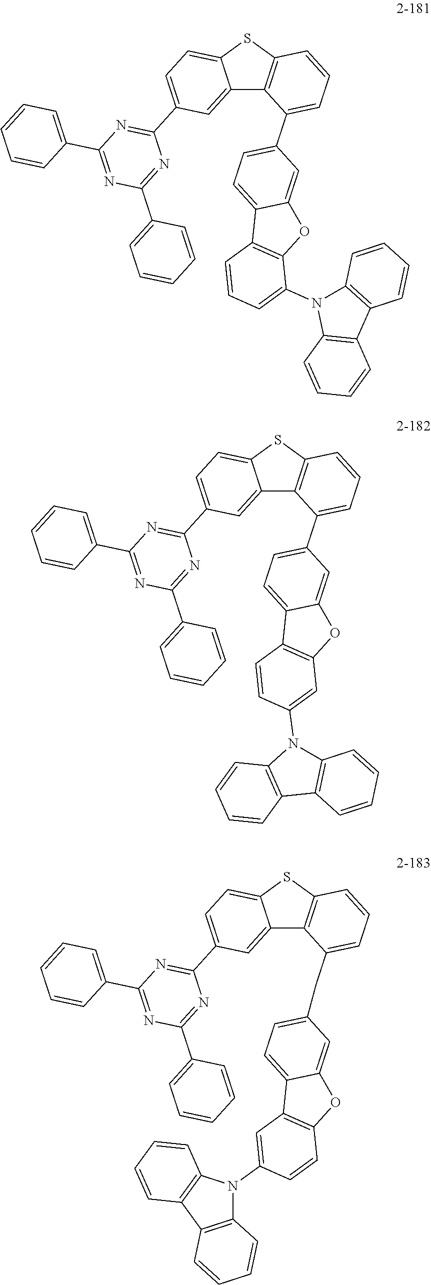 [SMILES: c1ccc(-c2nc(-c3ccccc3)nc(-c3ccc4sc5cccc(-c6ccc7c(c6)oc6c(-n8c9ccccc9c9ccccc98)cccc67)c5c4c3)n2)cc1 ; c1ccc(-c2nc(-c3ccccc3)nc(-c3ccc4sc5cccc(-c6ccc7c(c6)oc6cc(-n8c9ccccc9c9ccccc98)ccc67)c5c4c3)n2)cc1 ; c1ccc(-c2nc(-c3ccccc3)nc(-c3ccc4sc5cccc(Cc6ccc7c(c6)oc6ccc(-n8c9ccccc9c9ccccc98)cc67)c5c4c3)n2)cc1]

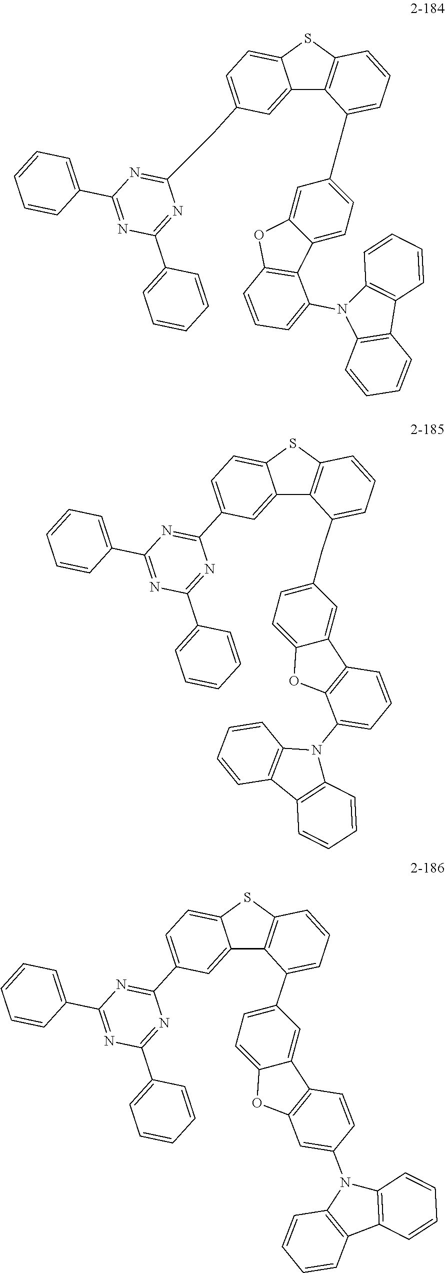 [SMILES: c1ccc(-c2nc(CCc3ccc4sc5cccc(Cc6ccc7c(c6)oc6cccc(-n8c9ccccc9c9ccccc98)c67)c5c4c3)nc(-c3ccccc3)n2)cc1 ; c1ccc(-c2nc(-c3ccccc3)nc(-c3ccc4sc5cccc(Cc6ccc7oc8c(-n9c%10ccccc%10c%10ccccc%109)cccc8c7c6)c5c4c3)n2)cc1 ; c1ccc(-c2nc(-c3ccccc3)nc(-c3ccc4sc5cccc(-c6ccc7oc8cc(-n9c%10ccccc%10c%10ccccc%109)ccc8c7c6)c5c4c3)n2)cc1]

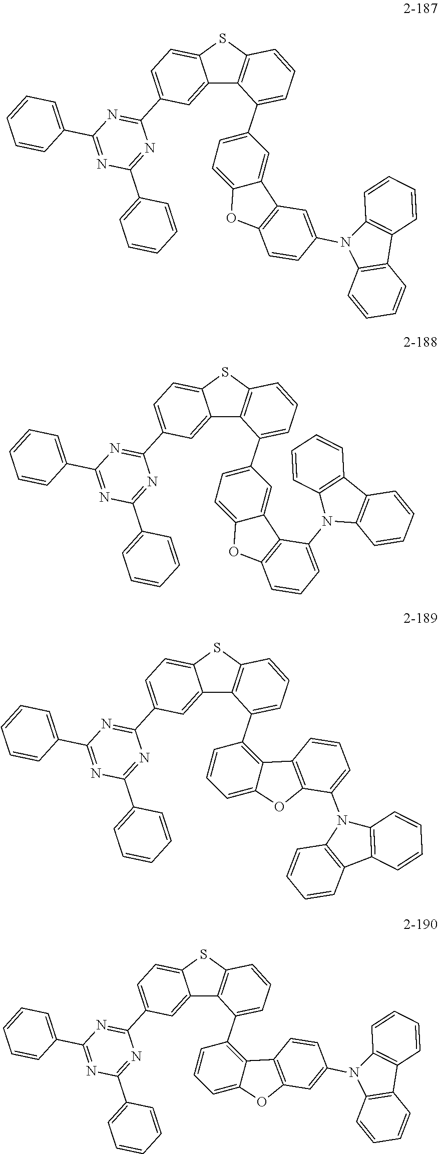 [SMILES: c1ccc(-c2nc(-c3ccccc3)nc(-c3ccc4sc5cccc(-c6ccc7oc8ccc(-n9c%10ccccc%10c%10ccccc%109)cc8c7c6)c5c4c3)n2)cc1 ; c1ccc(-c2nc(-c3ccccc3)nc(-c3ccc4sc5cccc(-c6ccc7oc8cccc(-n9c%10ccccc%10c%10ccccc%109)c8c7c6)c5c4c3)n2)cc1 ; c1ccc(-c2nc(-c3ccccc3)nc(-c3ccc4sc5cccc(-c6cccc7oc8c(-n9c%10ccccc%10c%10ccccc%109)cccc8c67)c5c4c3)n2)cc1 ; c1ccc(-c2nc(-c3ccccc3)nc(-c3ccc4sc5cccc(-c6cccc7oc8cc(-n9c%10ccccc%10c%10ccccc%109)ccc8c67)c5c4c3)n2)cc1]

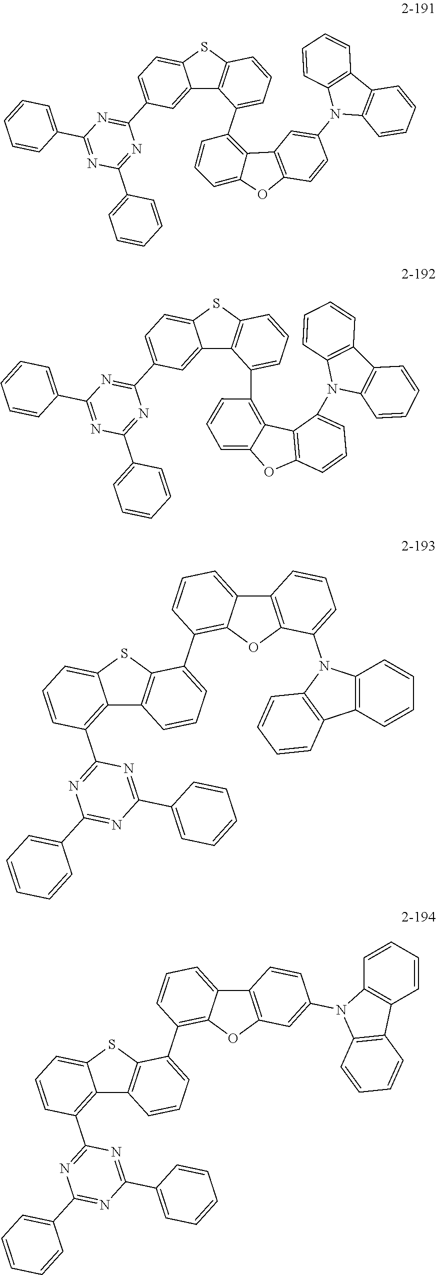 [SMILES: c1ccc(-c2nc(-c3ccccc3)nc(-c3ccc4sc5cccc(-c6cccc7oc8ccc(-n9c%10ccccc%10c%10ccccc%109)cc8c67)c5c4c3)n2)cc1 ; c1ccc(-c2nc(-c3ccccc3)nc(-c3ccc4sc5cccc(-c6cccc7oc8cccc(-n9c%10ccccc%10c%10ccccc%109)c8c67)c5c4c3)n2)cc1 ; c1ccc(-c2nc(-c3ccccc3)nc(-c3cccc4sc5c(-c6cccc7c6oc6c(-n8c9ccccc9c9ccccc98)cccc67)cccc5c34)n2)cc1 ; c1ccc(-c2nc(-c3ccccc3)nc(-c3cccc4sc5c(-c6cccc7c6oc6cc(-n8c9ccccc9c9ccccc98)ccc67)cccc5c34)n2)cc1]

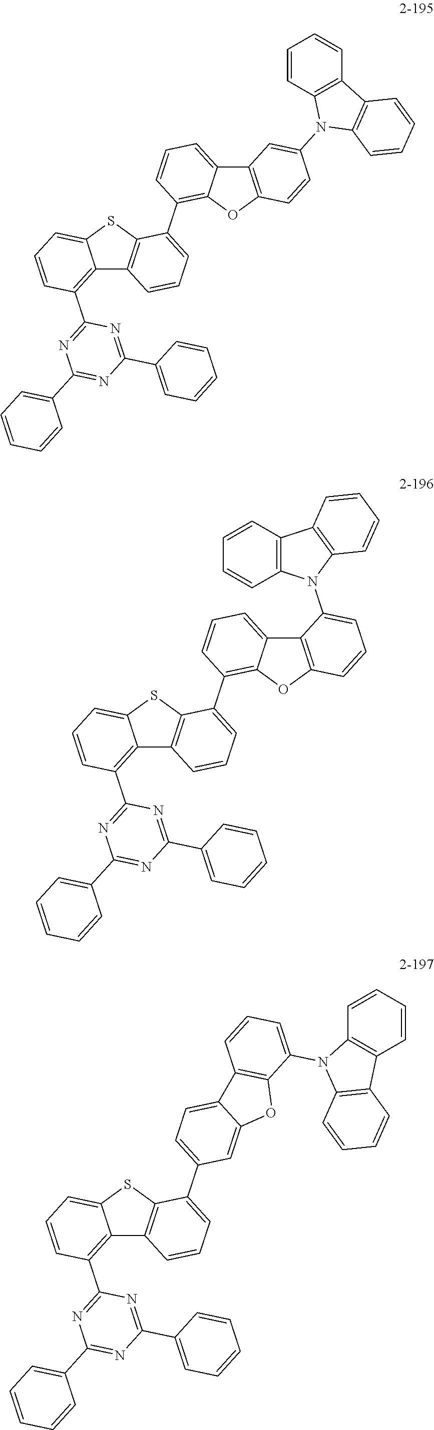 [SMILES: c1ccc(-c2nc(-c3ccccc3)nc(-c3cccc4sc5c(-c6cccc7c6oc6ccc(-n8c9ccccc9c9ccccc98)cc67)cccc5c34)n2)cc1 ; c1ccc(-c2nc(-c3ccccc3)nc(-c3cccc4sc5c(-c6cccc7c6oc6cccc(-n8c9ccccc9c9ccccc98)c67)cccc5c34)n2)cc1 ; c1ccc(-c2nc(-c3ccccc3)nc(-c3cccc4sc5c(-c6ccc7c(c6)oc6c(-n8c9ccccc9c9ccccc98)cccc67)cccc5c34)n2)cc1]

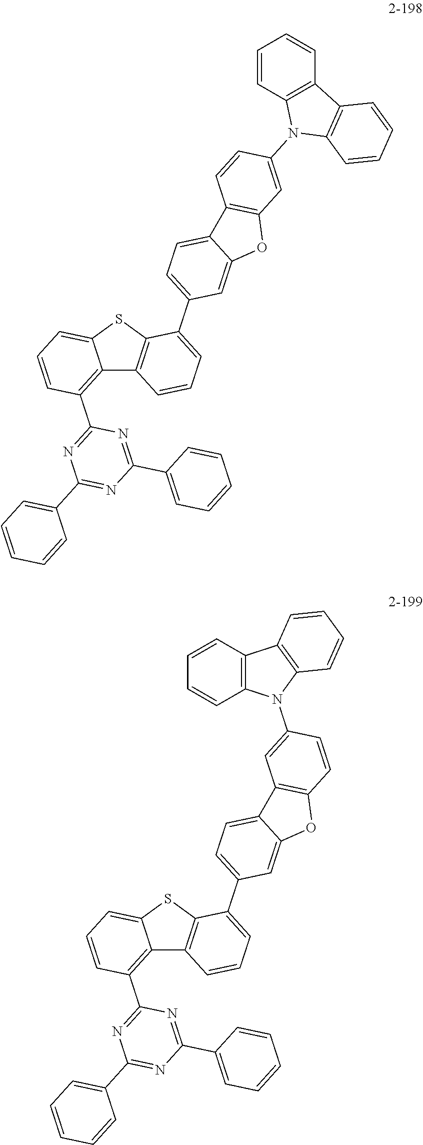 [SMILES: c1ccc(-c2nc(-c3ccccc3)nc(-c3cccc4sc5c(-c6ccc7c(c6)oc6cc(-n8c9ccccc9c9ccccc98)ccc67)cccc5c34)n2)cc1 ; c1ccc(-c2nc(-c3ccccc3)nc(-c3cccc4sc5c(-c6ccc7c(c6)oc6ccc(-n8c9ccccc9c9ccccc98)cc67)cccc5c34)n2)cc1]

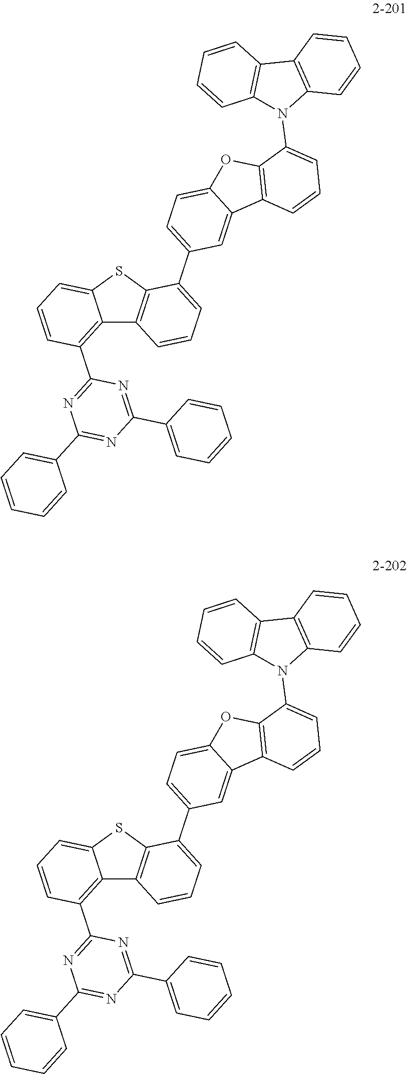 [SMILES: c1ccc(-c2nc(-c3ccccc3)nc(-c3cccc4sc5c(-c6ccc7oc8c(-n9c%10ccccc%10c%10ccccc%109)cccc8c7c6)cccc5c34)n2)cc1 ; c1ccc(-c2nc(-c3ccccc3)nc(-c3cccc4sc5c(-c6ccc7oc8c(-n9c%10ccccc%10c%10ccccc%109)cccc8c7c6)cccc5c34)n2)cc1]

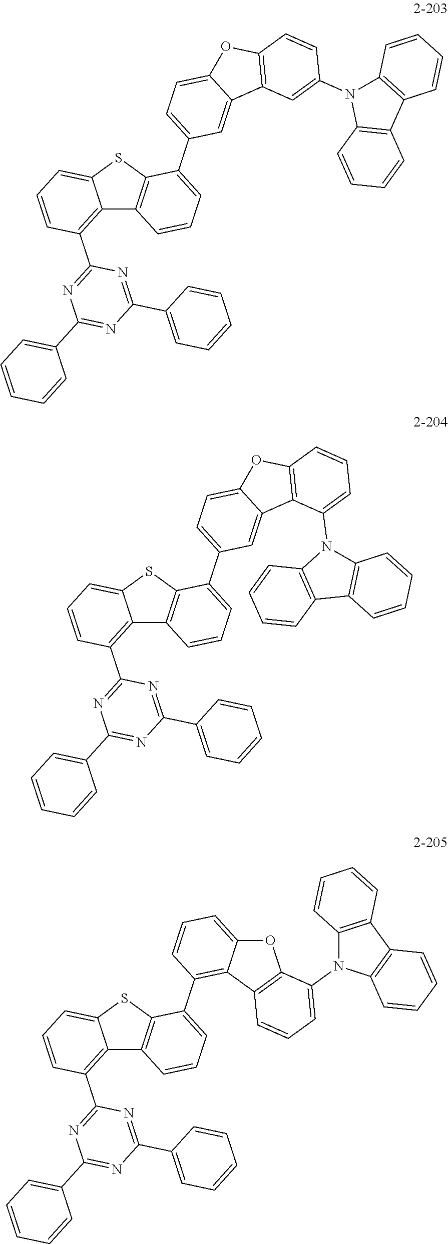 [SMILES: c1ccc(-c2nc(-c3ccccc3)nc(-c3cccc4sc5c(-c6ccc7oc8ccc(-n9c%10ccccc%10c%10ccccc%109)cc8c7c6)cccc5c34)n2)cc1 ; c1ccc(-c2nc(-c3ccccc3)nc(-c3cccc4sc5c(-c6ccc7oc8cccc(-n9c%10ccccc%10c%10ccccc%109)c8c7c6)cccc5c34)n2)cc1 ; c1ccc(-c2nc(-c3ccccc3)nc(-c3cccc4sc5c(-c6cccc7oc8c(-n9c%10ccccc%10c%10ccccc%109)cccc8c67)cccc5c34)n2)cc1]

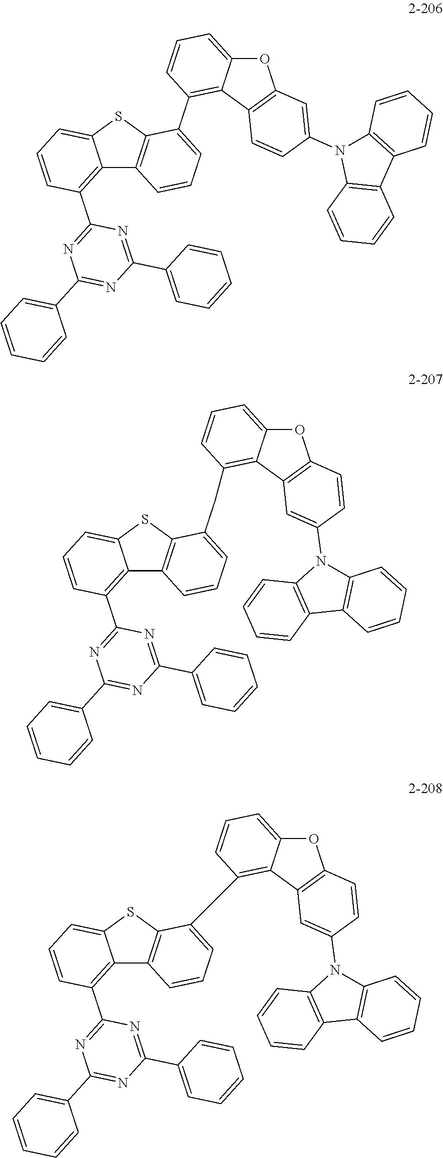 [SMILES: c1ccc(-c2nc(-c3ccccc3)nc(-c3cccc4sc5c(-c6cccc7oc8cc(-n9c%10ccccc%10c%10ccccc%109)ccc8c67)cccc5c34)n2)cc1 ; c1ccc(-c2nc(-c3ccccc3)nc(-c3cccc4sc5c(Cc6cccc7oc8ccc(-n9c%10ccccc%10c%10ccccc%109)cc8c67)cccc5c34)n2)cc1 ; c1ccc(-c2nc(-c3ccccc3)nc(-c3cccc4sc5c(Cc6cccc7oc8ccc(-n9c%10ccccc%10c%10ccccc%109)cc8c67)cccc5c34)n2)cc1]

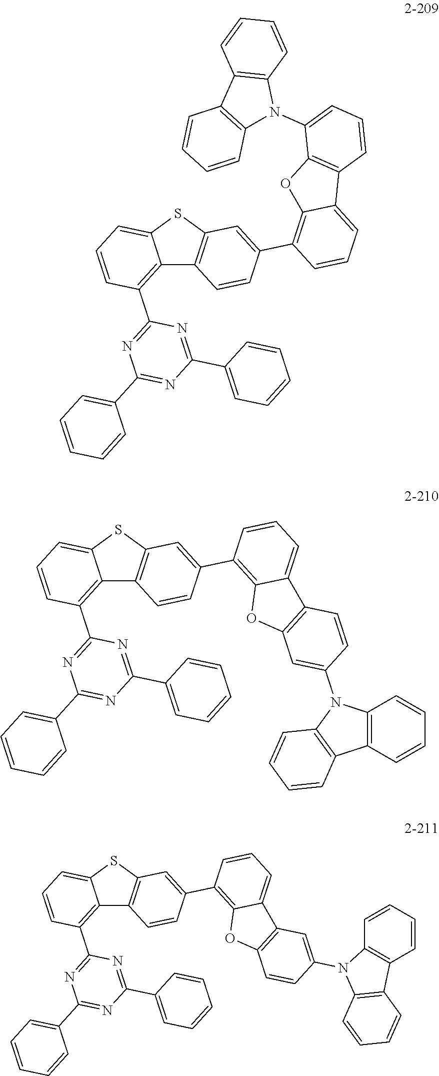 [SMILES: c1ccc(-c2nc(-c3ccccc3)nc(-c3cccc4sc5cc(-c6cccc7c6oc6c(-n8c9ccccc9c9ccccc98)cccc67)ccc5c34)n2)cc1 ; c1ccc(-c2nc(-c3ccccc3)nc(-c3cccc4sc5cc(-c6cccc7c6oc6cc(-n8c9ccccc9c9ccccc98)ccc67)ccc5c34)n2)cc1 ; c1ccc(-c2nc(-c3ccccc3)nc(-c3cccc4sc5cc(-c6cccc7c6oc6ccc(-n8c9ccccc9c9ccccc98)cc67)ccc5c34)n2)cc1]

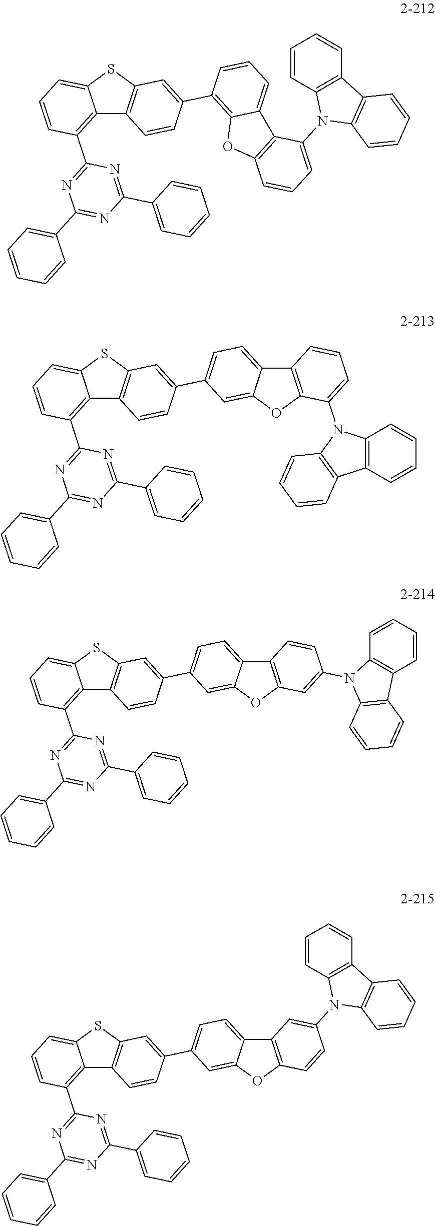 [SMILES: c1ccc(-c2nc(-c3ccccc3)nc(-c3cccc4sc5cc(-c6cccc7c6oc6cccc(-n8c9ccccc9c9ccccc98)c67)ccc5c34)n2)cc1 ; c1ccc(-c2nc(-c3ccccc3)nc(-c3cccc4sc5cc(-c6ccc7c(c6)oc6c(-n8c9ccccc9c9ccccc98)cccc67)ccc5c34)n2)cc1 ; c1ccc(-c2nc(-c3ccccc3)nc(-c3cccc4sc5cc(-c6ccc7c(c6)oc6cc(-n8c9ccccc9c9ccccc98)ccc67)ccc5c34)n2)cc1 ; c1ccc(-c2nc(-c3ccccc3)nc(-c3cccc4sc5cc(-c6ccc7c(c6)oc6ccc(-n8c9ccccc9c9ccccc98)cc67)ccc5c34)n2)cc1]

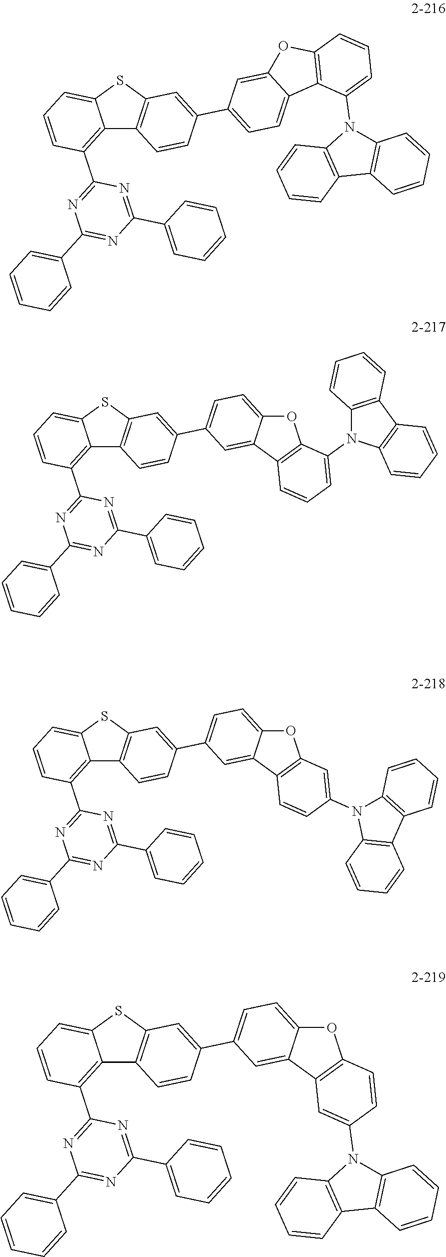 [SMILES: c1ccc(-c2nc(-c3ccccc3)nc(-c3cccc4sc5cc(-c6ccc7c(c6)oc6cccc(-n8c9ccccc9c9ccccc98)c67)ccc5c34)n2)cc1 ; c1ccc(-c2nc(-c3ccccc3)nc(-c3cccc4sc5cc(-c6ccc7oc8c(-n9c%10ccccc%10c%10ccccc%109)cccc8c7c6)ccc5c34)n2)cc1 ; c1ccc(-c2nc(-c3ccccc3)nc(-c3cccc4sc5cc(-c6ccc7oc8cc(-n9c%10ccccc%10c%10ccccc%109)ccc8c7c6)ccc5c34)n2)cc1 ; c1ccc(-c2nc(-c3ccccc3)nc(-c3cccc4sc5cc(-c6ccc7oc8ccc(-n9c%10ccccc%10c%10ccccc%109)cc8c7c6)ccc5c34)n2)cc1]

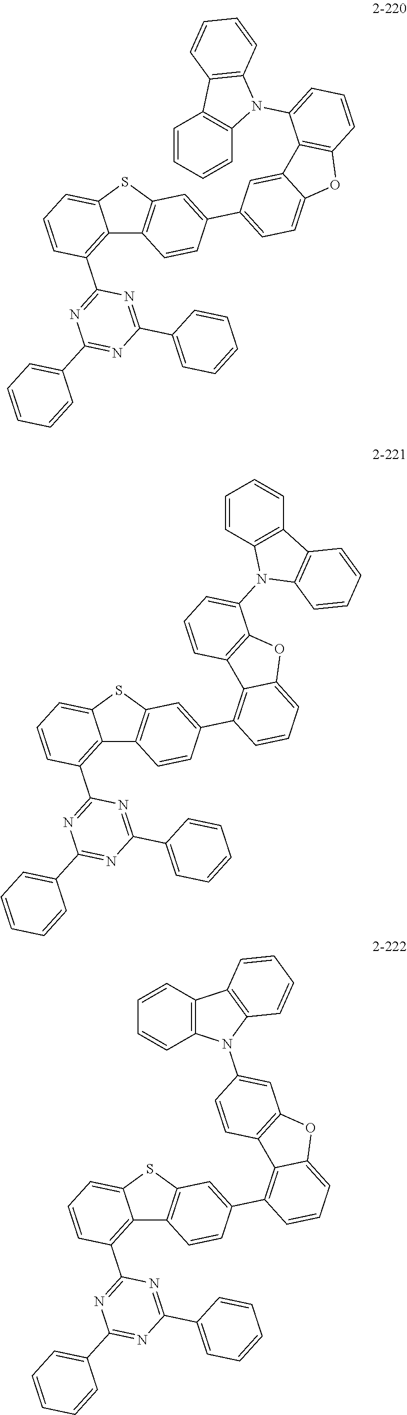 [SMILES: c1ccc(-c2nc(-c3ccccc3)nc(-c3cccc4sc5cc(-c6ccc7oc8cccc(-n9c%10ccccc%10c%10ccccc%109)c8c7c6)ccc5c34)n2)cc1 ; c1ccc(-c2nc(-c3ccccc3)nc(-c3cccc4sc5cc(-c6cccc7oc8c(-n9c%10ccccc%10c%10ccccc%109)cccc8c67)ccc5c34)n2)cc1 ; c1ccc(-c2nc(-c3ccccc3)nc(-c3cccc4sc5cc(-c6cccc7oc8cc(-n9c%10ccccc%10c%10ccccc%109)ccc8c67)ccc5c34)n2)cc1]

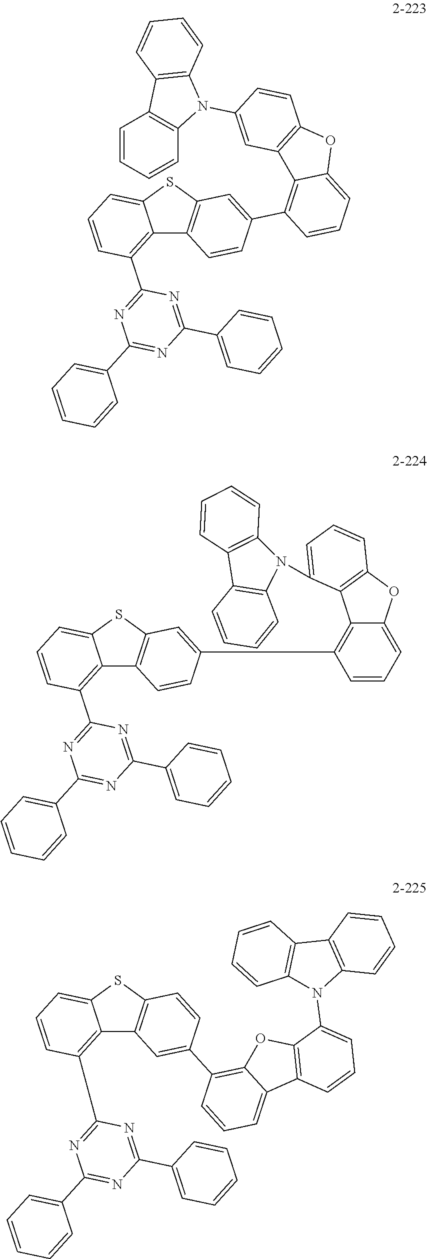 [SMILES: c1ccc(-c2nc(-c3ccccc3)nc(-c3cccc4sc5cc(-c6cccc7oc8ccc(-n9c%10ccccc%10c%10ccccc%109)cc8c67)ccc5c34)n2)cc1 ; c1ccc(-c2nc(-c3ccccc3)nc(-c3cccc4sc5cc(CCCc6cccc7oc8cccc(-n9c%10ccccc%10c%10ccccc%109)c8c67)ccc5c34)n2)cc1 ; c1ccc(-c2nc(Cc3cccc4sc5ccc(-c6cccc7c6oc6c(-n8c9ccccc9c9ccccc98)cccc67)cc5c34)nc(-c3ccccc3)n2)cc1]

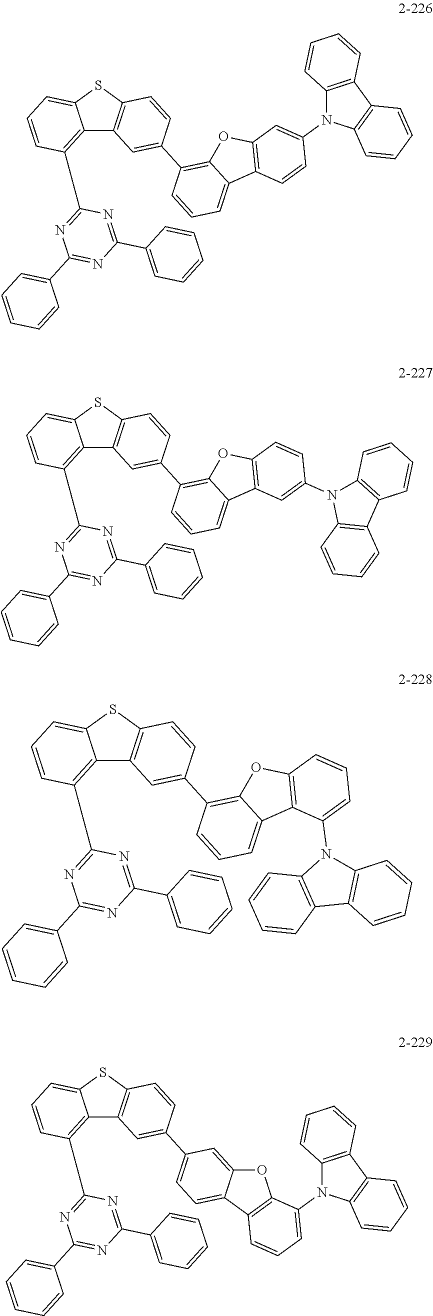 [SMILES: c1ccc(-c2nc(Cc3cccc4sc5ccc(-c6cccc7c6oc6cc(-n8c9ccccc9c9ccccc98)ccc67)cc5c34)nc(-c3ccccc3)n2)cc1 ; c1ccc(-c2nc(Cc3cccc4sc5ccc(-c6cccc7c6oc6ccc(-n8c9ccccc9c9ccccc98)cc67)cc5c34)nc(-c3ccccc3)n2)cc1 ; c1ccc(-c2nc(Cc3cccc4sc5ccc(-c6cccc7c6oc6cccc(-n8c9ccccc9c9ccccc98)c67)cc5c34)nc(-c3ccccc3)n2)cc1 ; c1ccc(-c2nc(Cc3cccc4sc5ccc(-c6ccc7c(c6)oc6c(-n8c9ccccc9c9ccccc98)cccc67)cc5c34)nc(-c3ccccc3)n2)cc1]

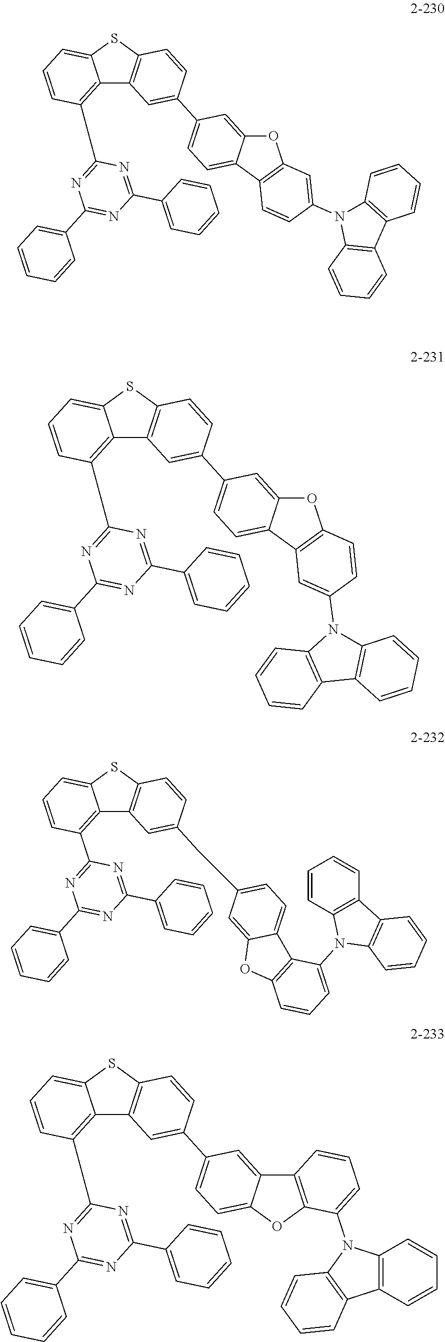 [SMILES: c1ccc(-c2nc(Cc3cccc4sc5ccc(-c6ccc7c(c6)oc6cc(-n8c9ccccc9c9ccccc98)ccc67)cc5c34)nc(-c3ccccc3)n2)cc1 ; c1ccc(-c2nc(Cc3cccc4sc5ccc(-c6ccc7c(c6)oc6ccc(-n8c9ccccc9c9ccccc98)cc67)cc5c34)nc(-c3ccccc3)n2)cc1 ; c1ccc(-c2nc(-c3ccccc3)nc(-c3cccc4sc5ccc(CCc6ccc7c(c6)oc6cccc(-n8c9ccccc9c9ccccc98)c67)cc5c34)n2)cc1 ; c1ccc(-c2nc(Cc3cccc4sc5ccc(-c6ccc7oc8c(-n9c%10ccccc%10c%10ccccc%109)cccc8c7c6)cc5c34)nc(-c3ccccc3)n2)cc1]

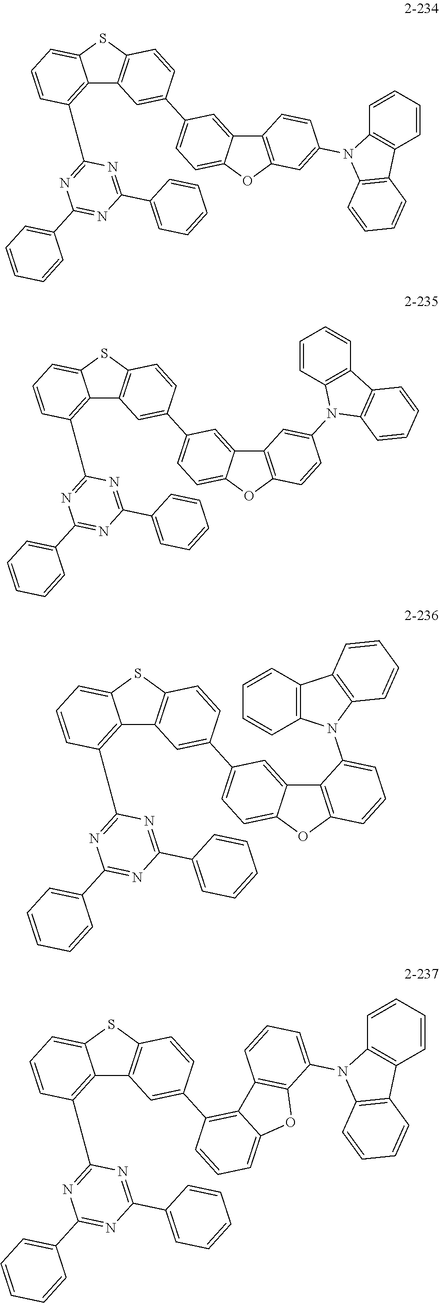 [SMILES: c1ccc(-c2nc(Cc3cccc4sc5ccc(-c6ccc7oc8cc(-n9c%10ccccc%10c%10ccccc%109)ccc8c7c6)cc5c34)nc(-c3ccccc3)n2)cc1 ; c1ccc(-c2nc(Cc3cccc4sc5ccc(-c6ccc7oc8ccc(-n9c%10ccccc%10c%10ccccc%109)cc8c7c6)cc5c34)nc(-c3ccccc3)n2)cc1 ; c1ccc(-c2nc(Cc3cccc4sc5ccc(-c6ccc7oc8cccc(-n9c%10ccccc%10c%10ccccc%109)c8c7c6)cc5c34)nc(-c3ccccc3)n2)cc1 ; c1ccc(-c2nc(Cc3cccc4sc5ccc(-c6cccc7oc8c(-n9c%10ccccc%10c%10ccccc%109)cccc8c67)cc5c34)nc(-c3ccccc3)n2)cc1]

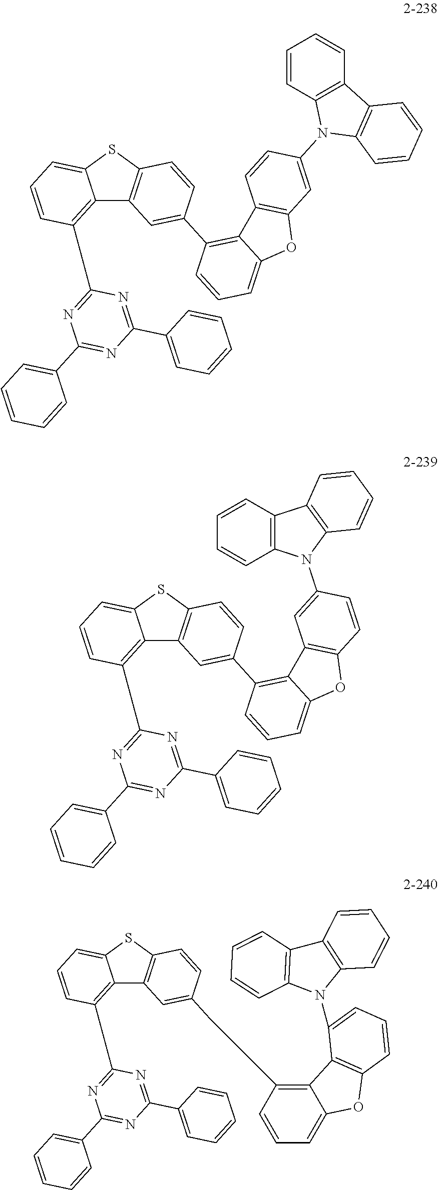 [SMILES: c1ccc(-c2nc(Cc3cccc4sc5ccc(-c6cccc7oc8cc(-n9c%10ccccc%10c%10ccccc%109)ccc8c67)cc5c34)nc(-c3ccccc3)n2)cc1 ; c1ccc(-c2nc(Cc3cccc4sc5ccc(-c6cccc7oc8ccc(-n9c%10ccccc%10c%10ccccc%109)cc8c67)cc5c34)nc(-c3ccccc3)n2)cc1 ; c1ccc(-c2nc(Cc3cccc4sc5ccc(CCCc6cccc7oc8cccc(-n9c%10ccccc%10c%10ccccc%109)c8c67)cc5c34)nc(-c3ccccc3)n2)cc1]

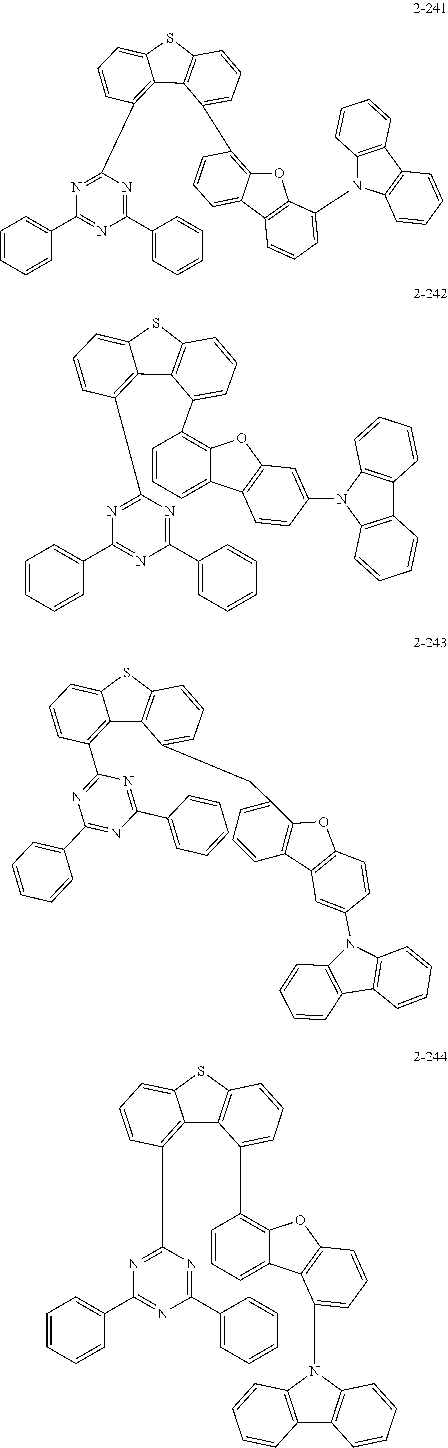 [SMILES: c1ccc(-c2nc(-c3ccccc3)nc(-c3cccc4sc5cccc(-c6cccc7c6oc6c(-n8c9ccccc9c9ccccc98)cccc67)c5c34)n2)cc1 ; c1ccc(-c2nc(CCc3cccc4sc5cccc(-c6cccc7c6oc6cc(-n8c9ccccc9c9ccccc98)ccc67)c5c34)nc(-c3ccccc3)n2)cc1 ; c1ccc(-c2nc(-c3ccccc3)nc(-c3cccc4sc5cccc(Cc6cccc7c6oc6ccc(-n8c9ccccc9c9ccccc98)cc67)c5c34)n2)cc1 ; c1ccc(-c2nc(CCc3cccc4sc5cccc(Cc6cccc7c6oc6cccc(Cn8c9ccccc9c9ccccc98)c67)c5c34)nc(-c3ccccc3)n2)cc1]

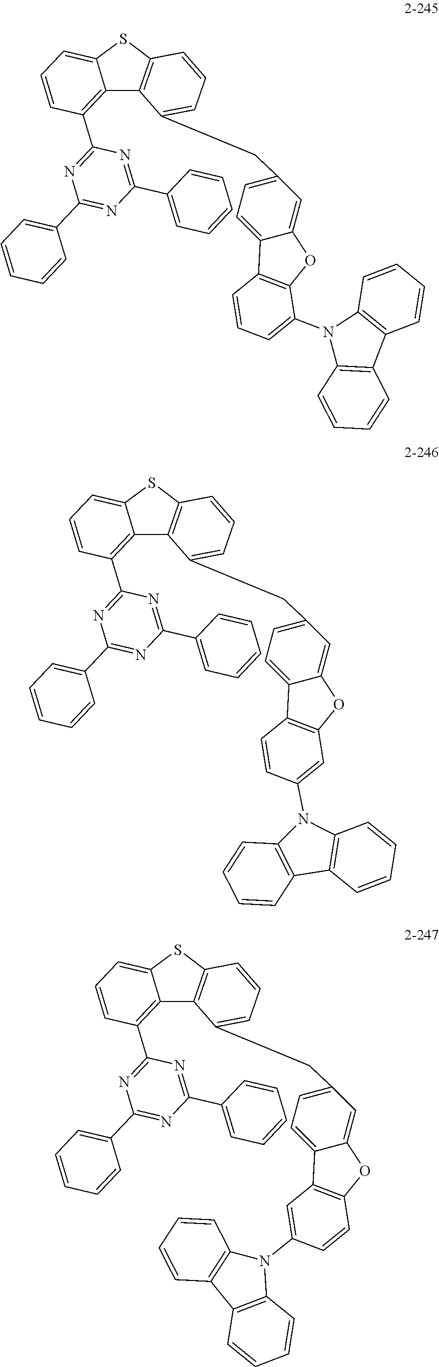 [SMILES: c1ccc(-c2nc(-c3ccccc3)nc(-c3cccc4sc5cccc(Cc6ccc7c(c6)oc6c(-n8c9ccccc9c9ccccc98)cccc67)c5c34)n2)cc1 ; c1ccc(-c2nc(-c3ccccc3)nc(-c3cccc4sc5cccc(Cc6ccc7c(c6)oc6cc(-n8c9ccccc9c9ccccc98)ccc67)c5c34)n2)cc1 ; c1ccc(-c2nc(-c3ccccc3)nc(-c3cccc4sc5cccc(Cc6ccc7c(c6)oc6ccc(-n8c9ccccc9c9ccccc98)cc67)c5c34)n2)cc1]

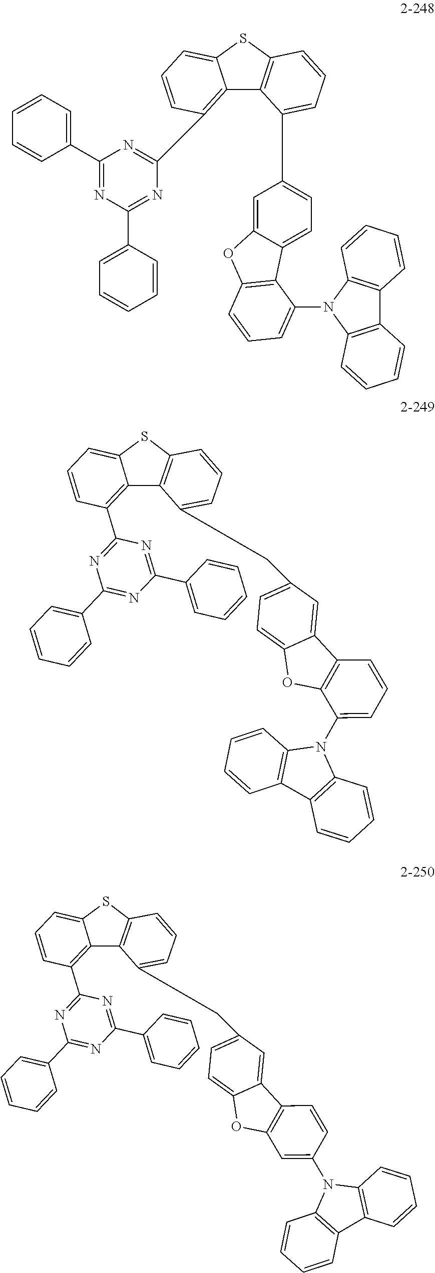 [SMILES: c1ccc(-c2nc(Cc3cccc4sc5cccc(Cc6ccc7c(c6)oc6cccc(-n8c9ccccc9c9ccccc98)c67)c5c34)nc(-c3ccccc3)n2)cc1 ; c1ccc(-c2nc(-c3ccccc3)nc(-c3cccc4sc5cccc(CCCc6ccc7oc8c(-n9c%10ccccc%10c%10ccccc%109)cccc8c7c6)c5c34)n2)cc1 ; c1ccc(-c2nc(-c3ccccc3)nc(-c3cccc4sc5cccc(CCCc6ccc7oc8cc(-n9c%10ccccc%10c%10ccccc%109)ccc8c7c6)c5c34)n2)cc1]

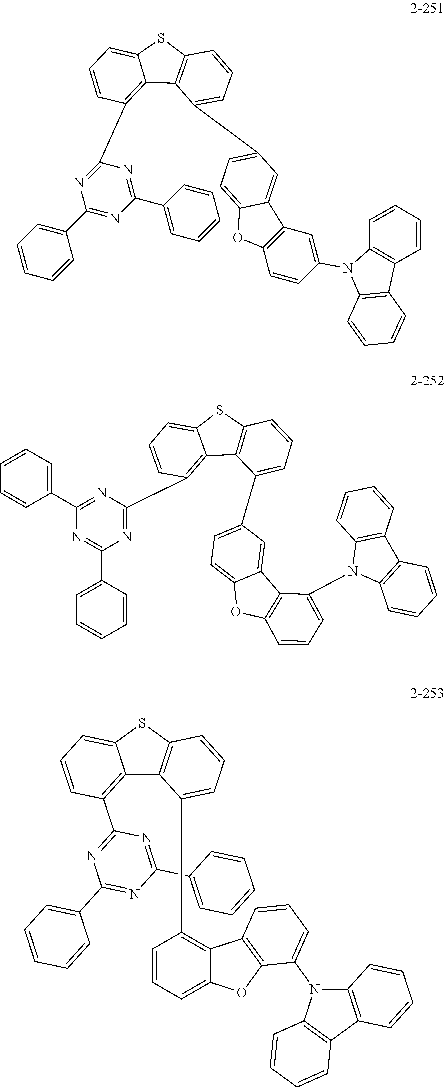

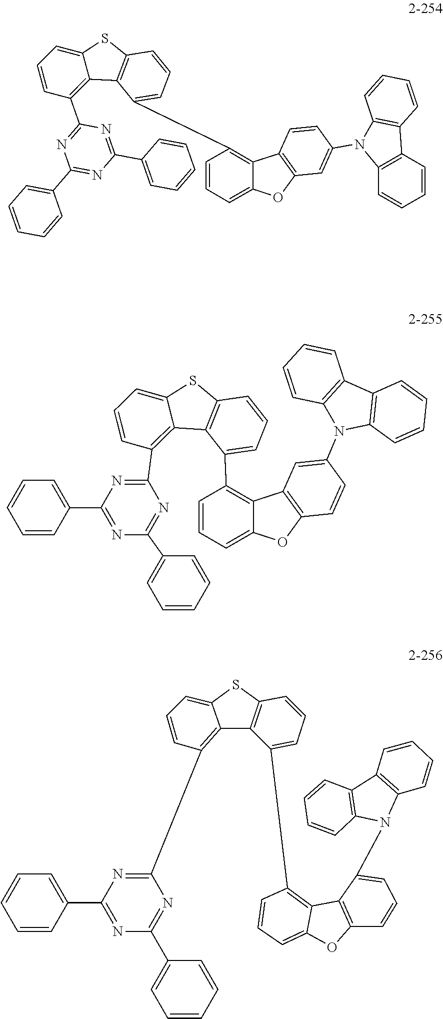

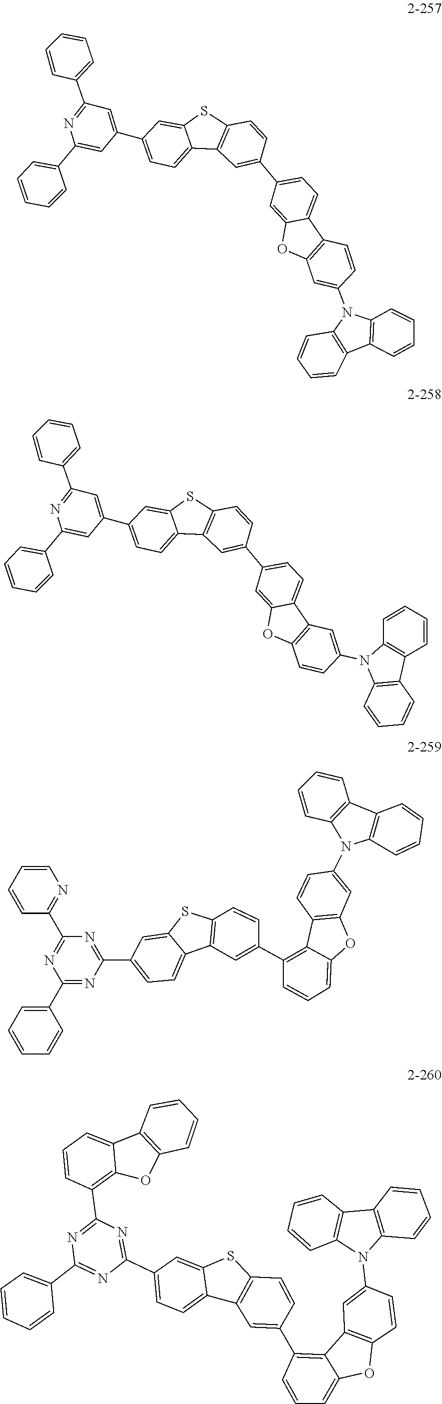

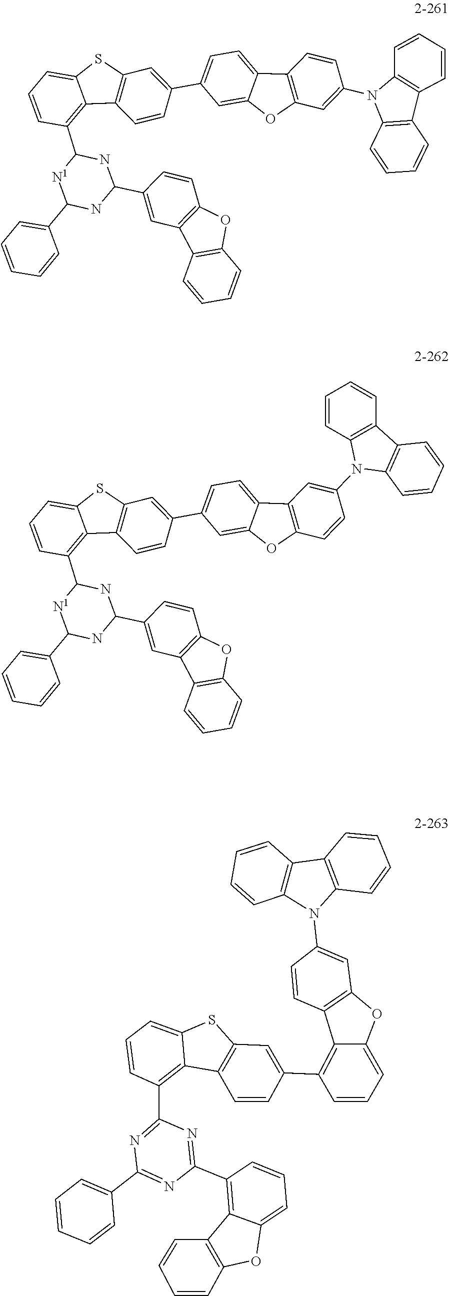

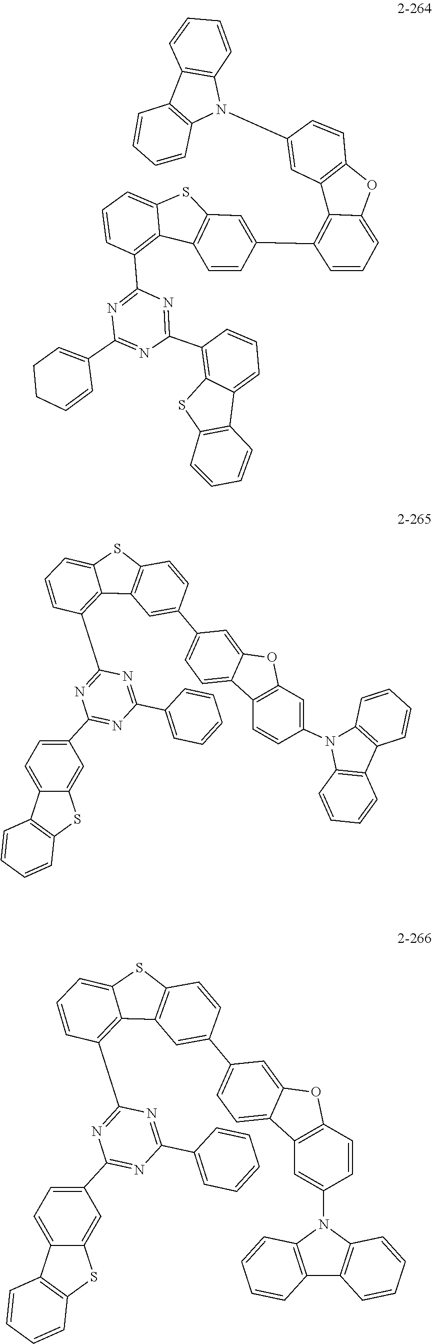

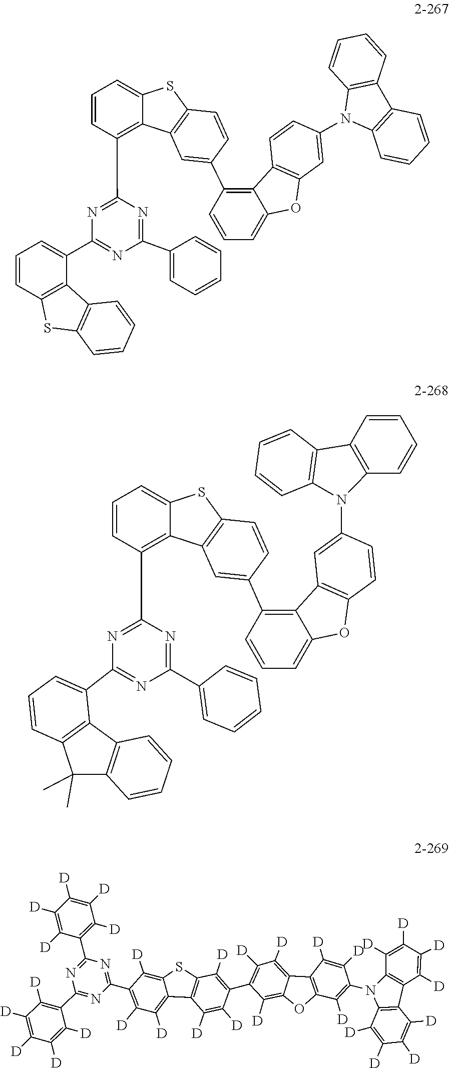

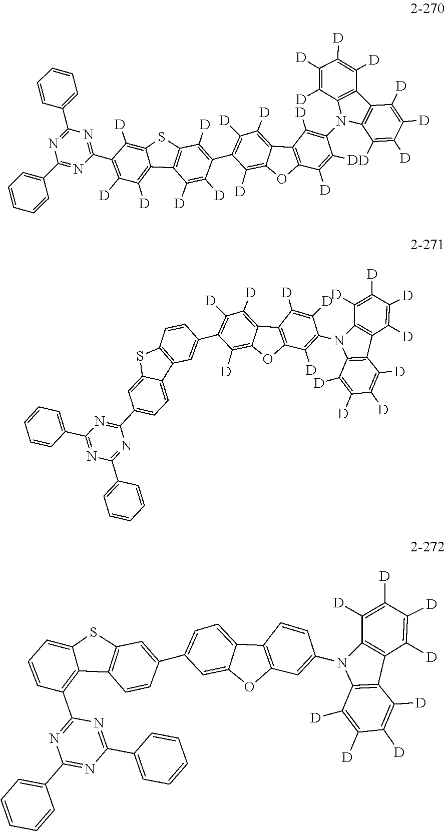

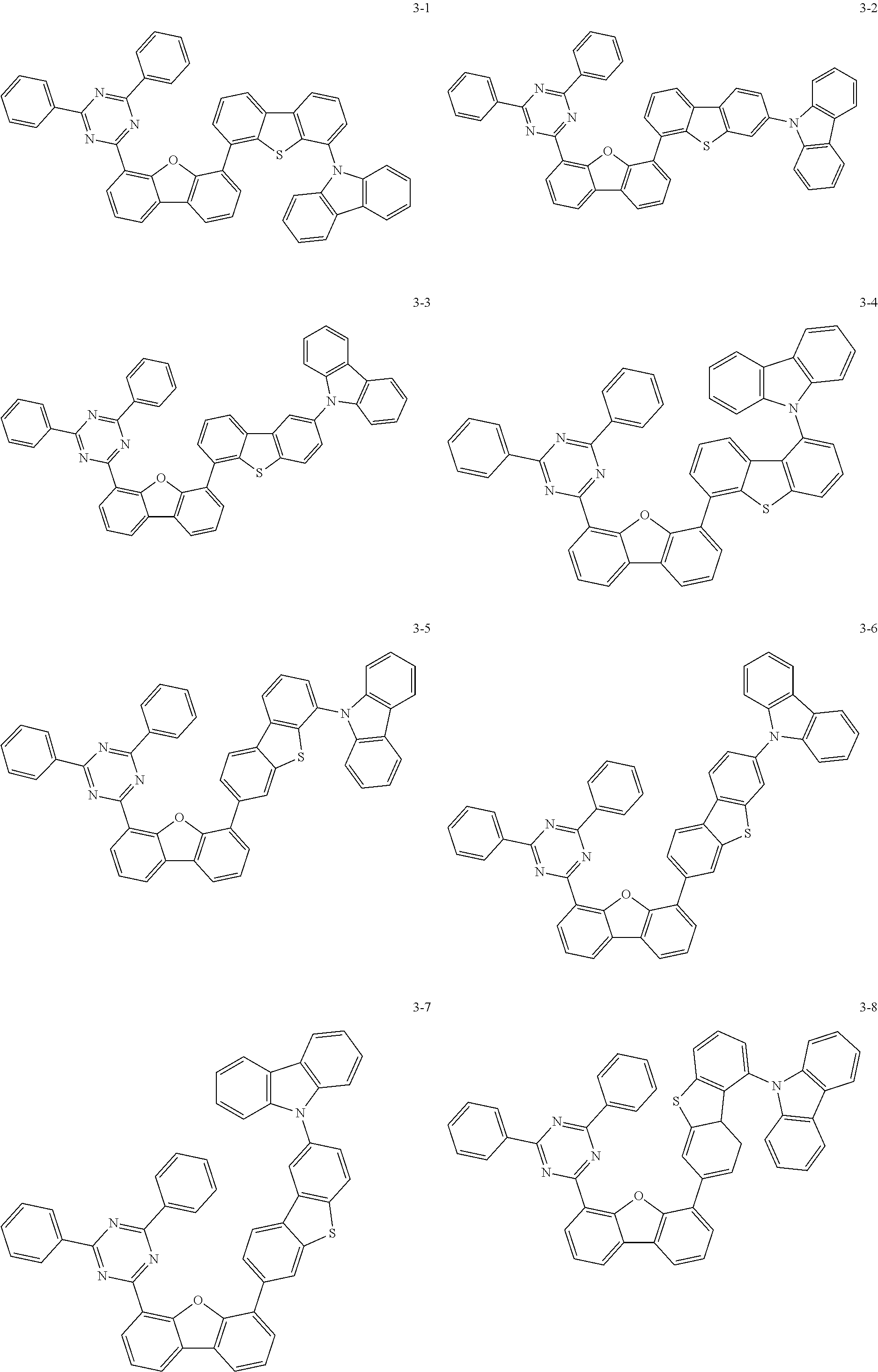

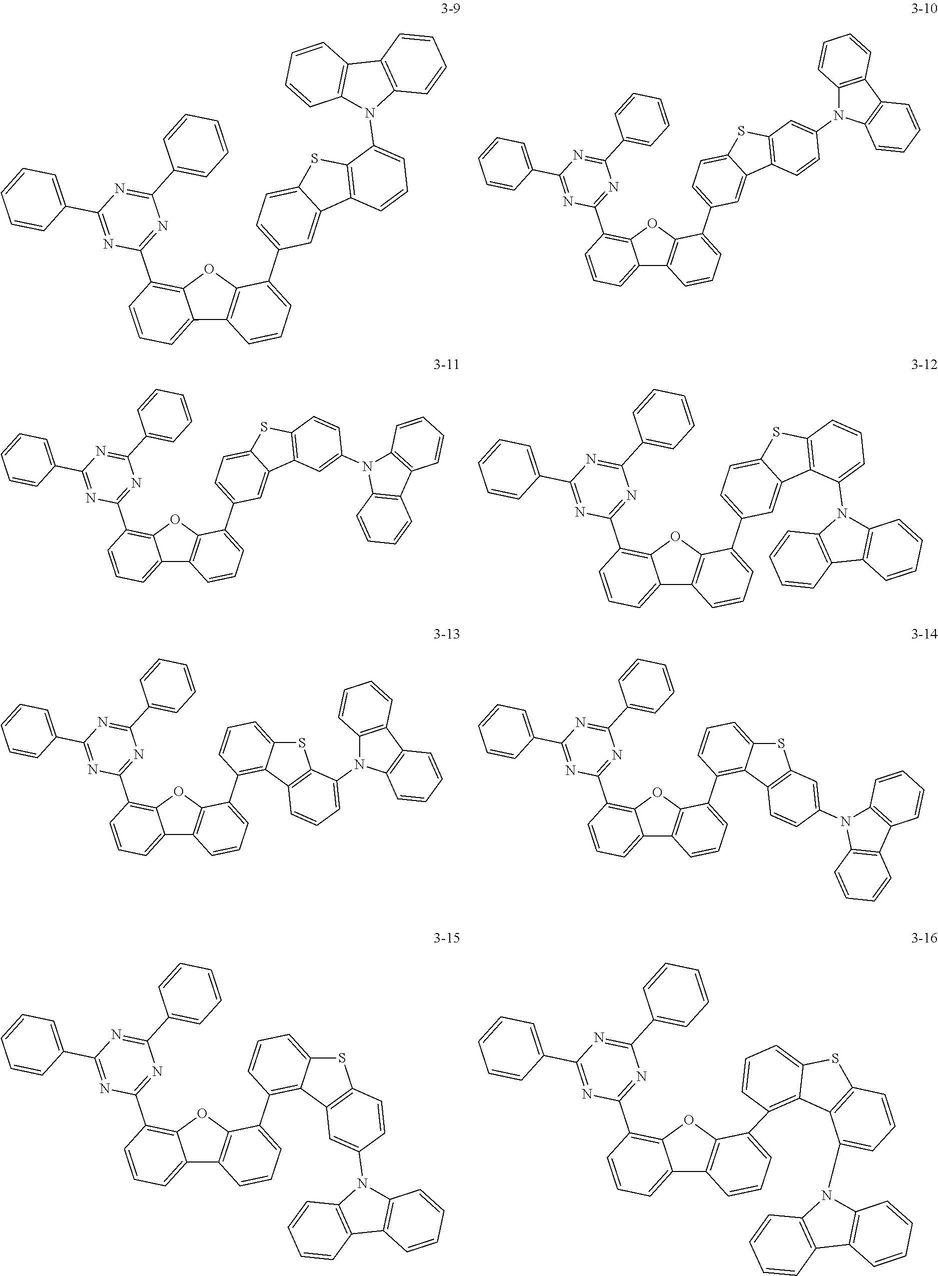

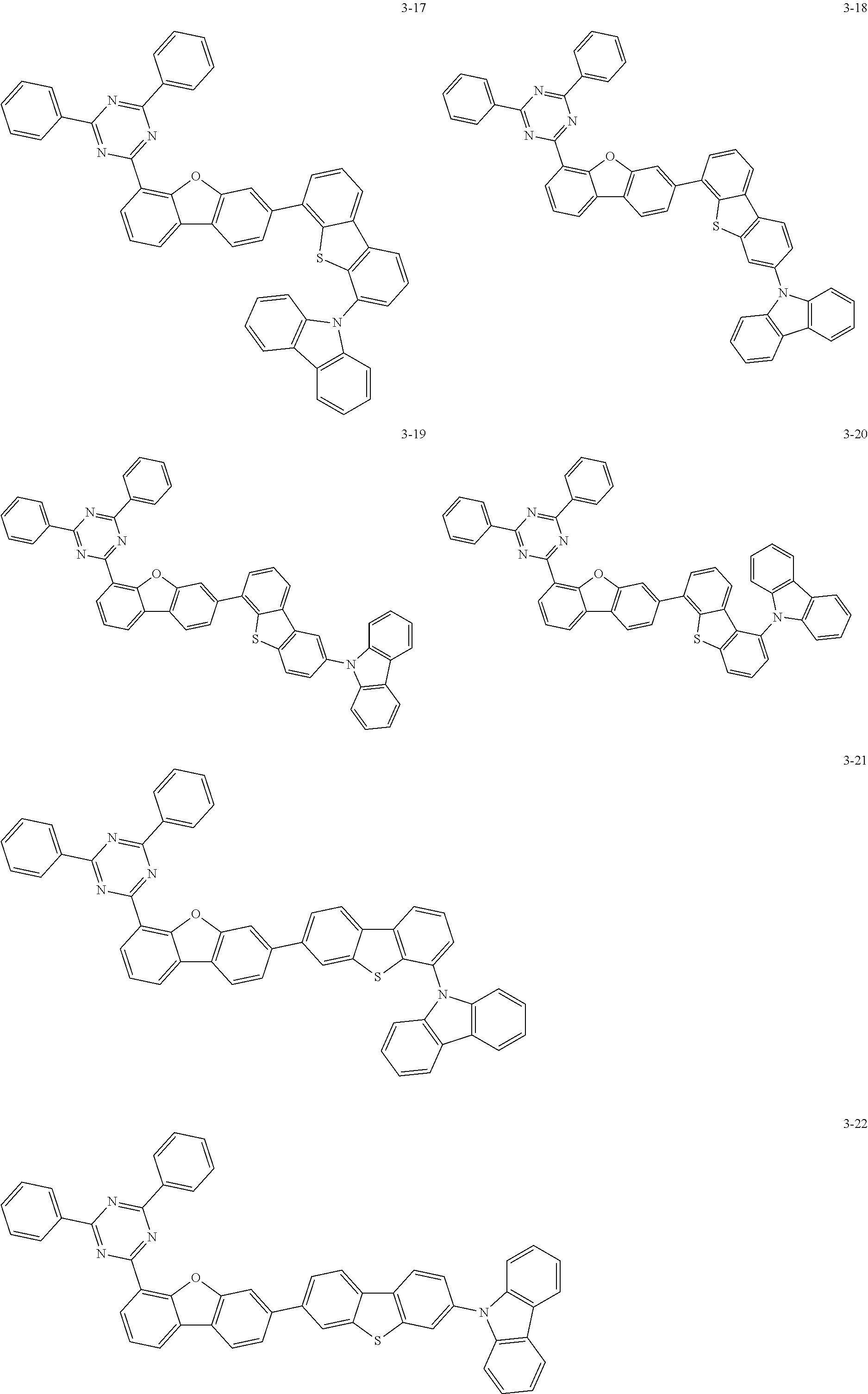

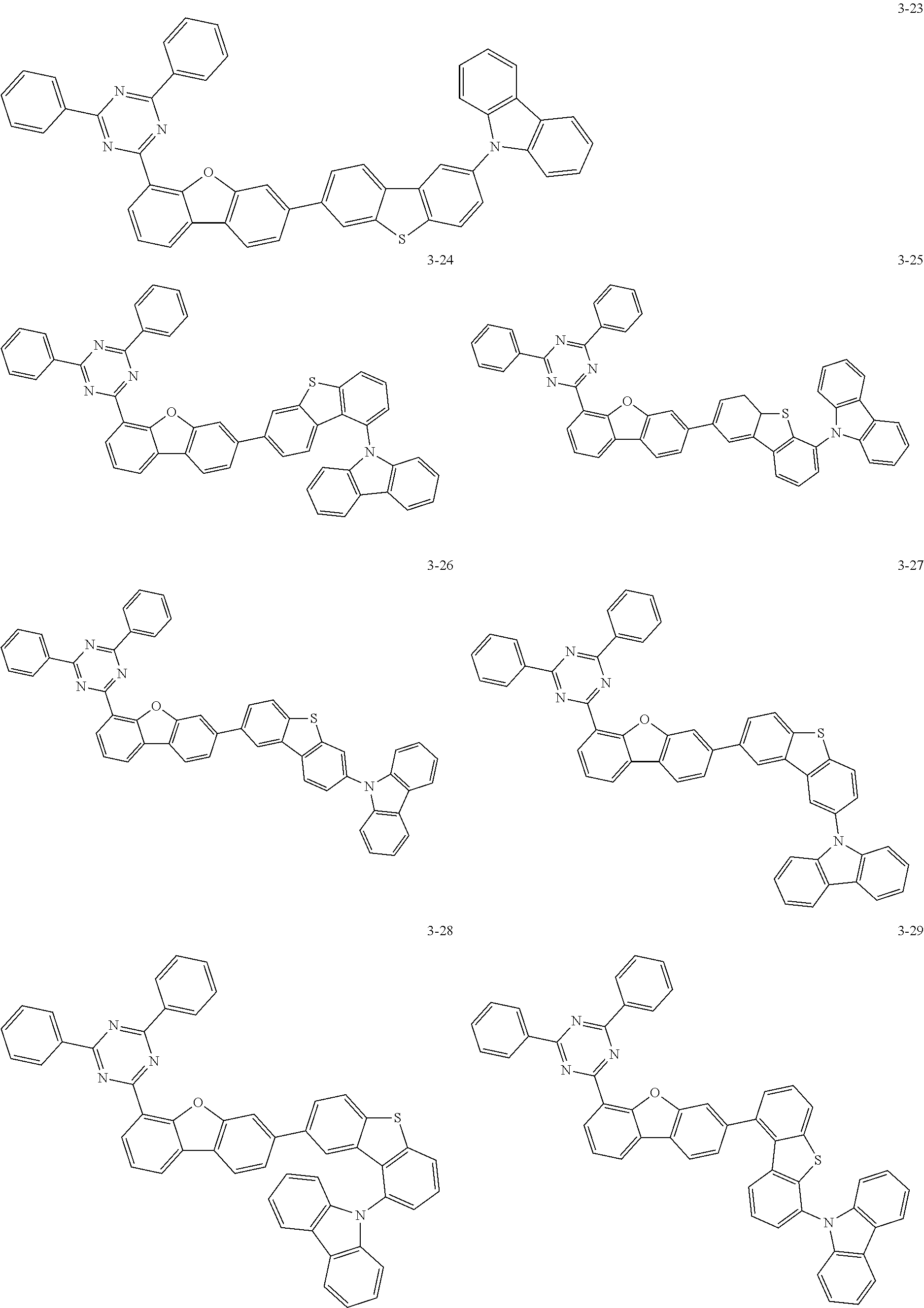

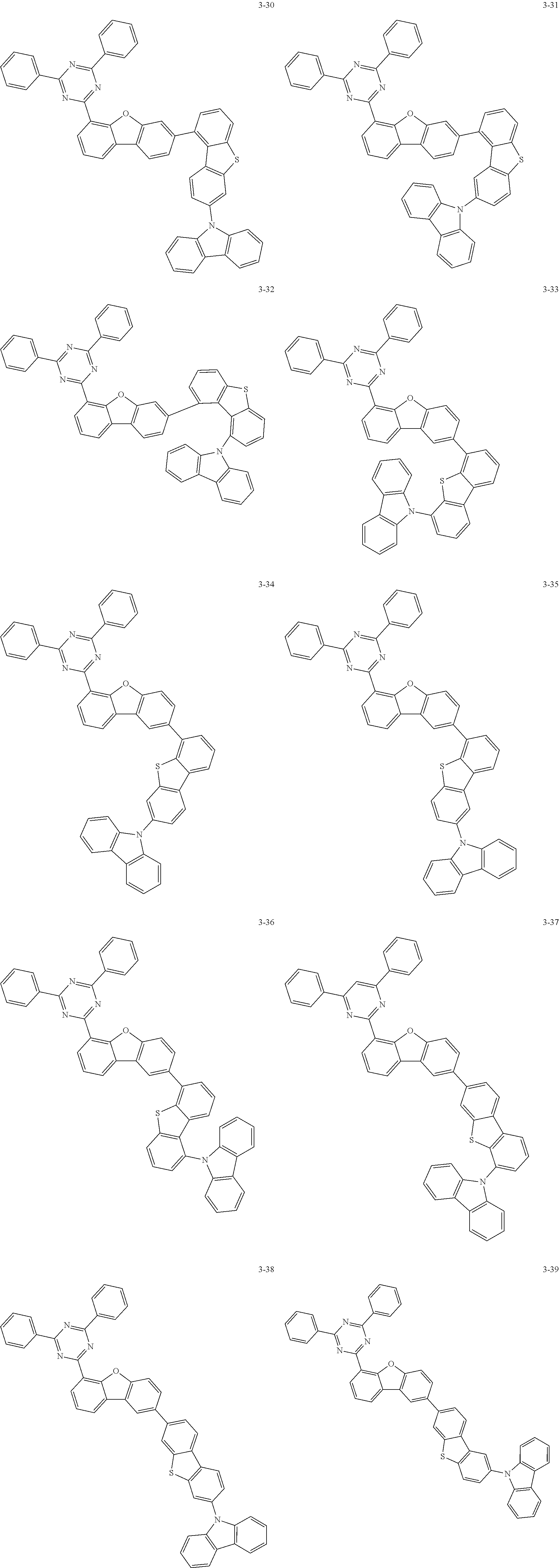

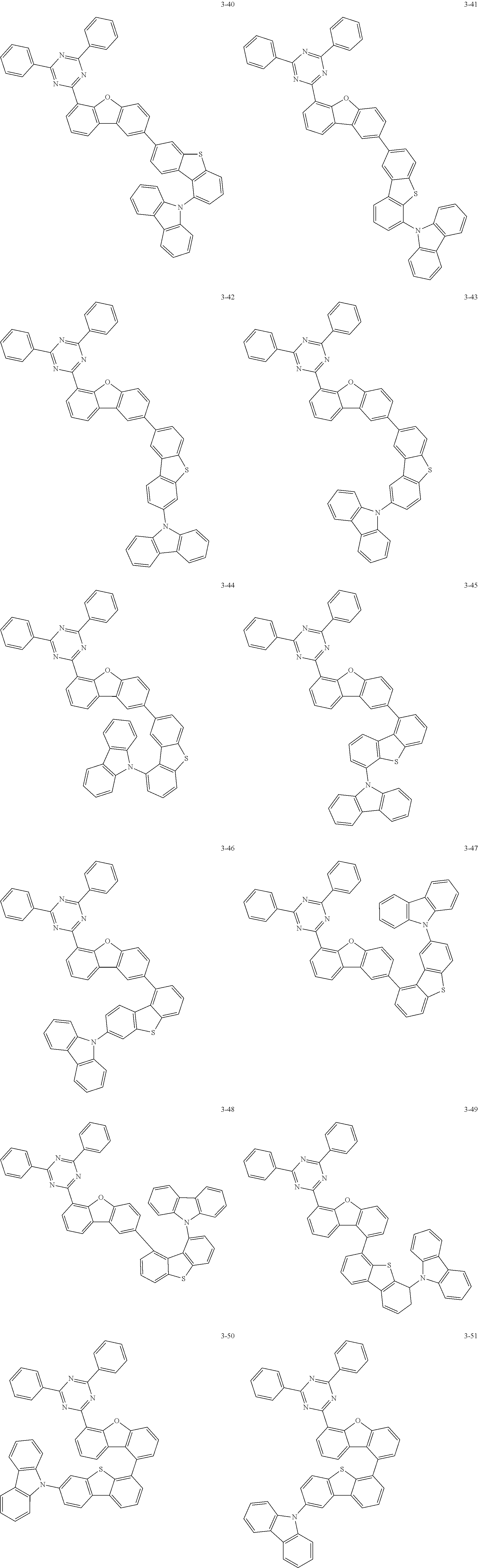

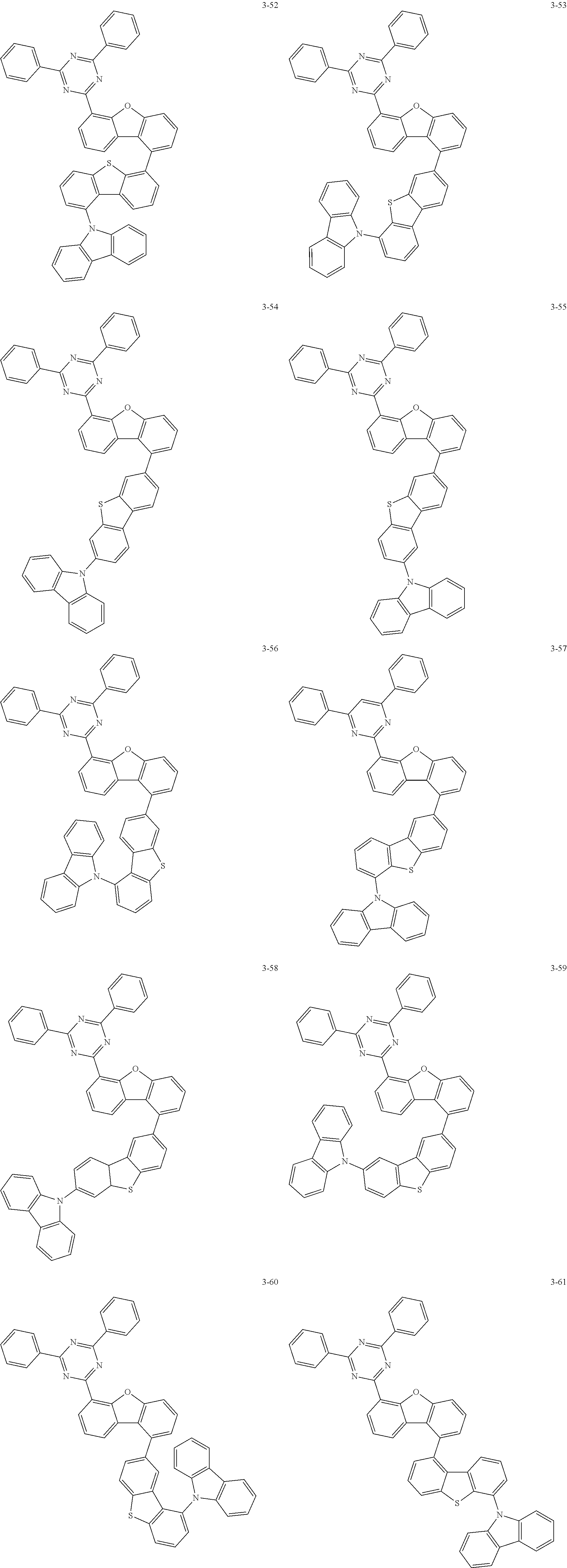

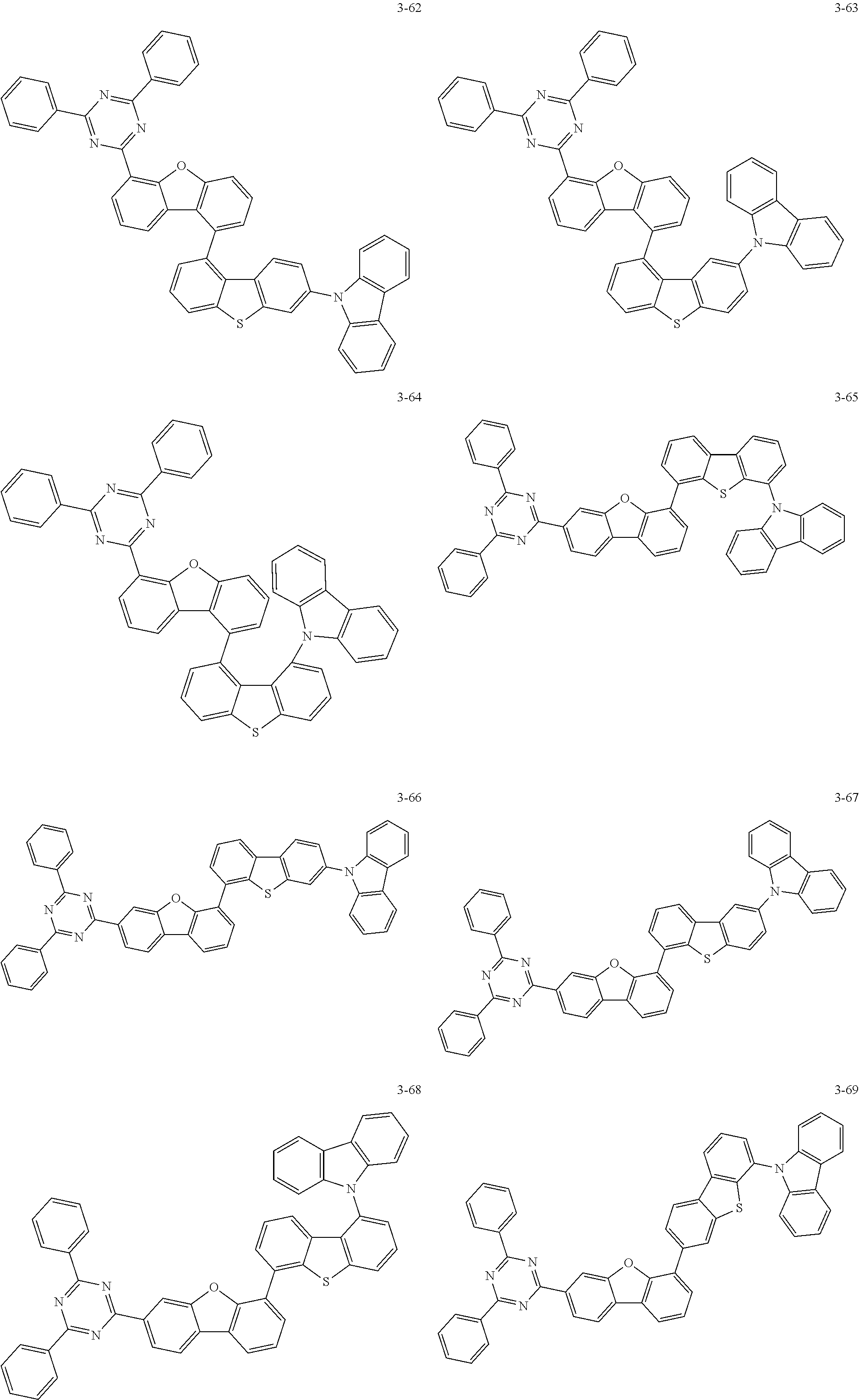

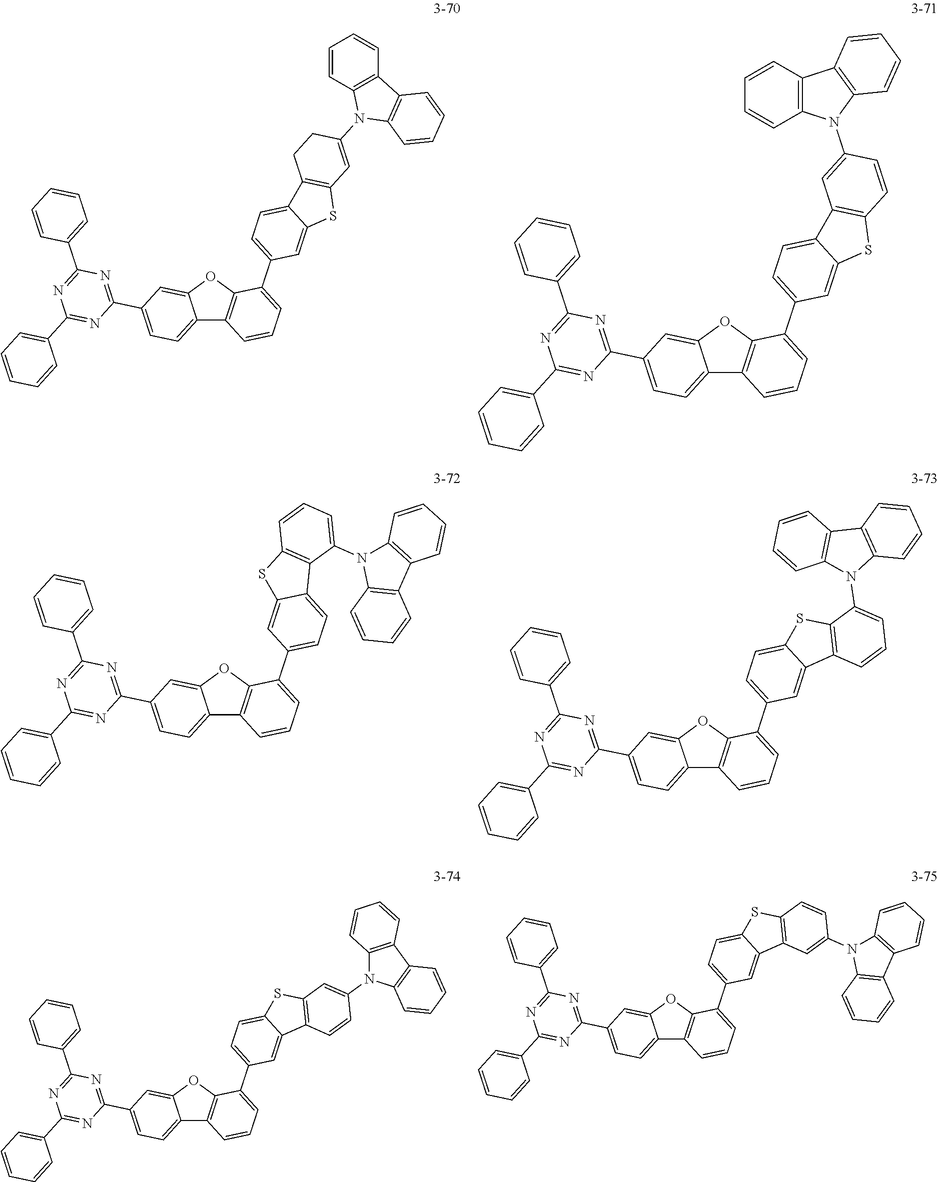

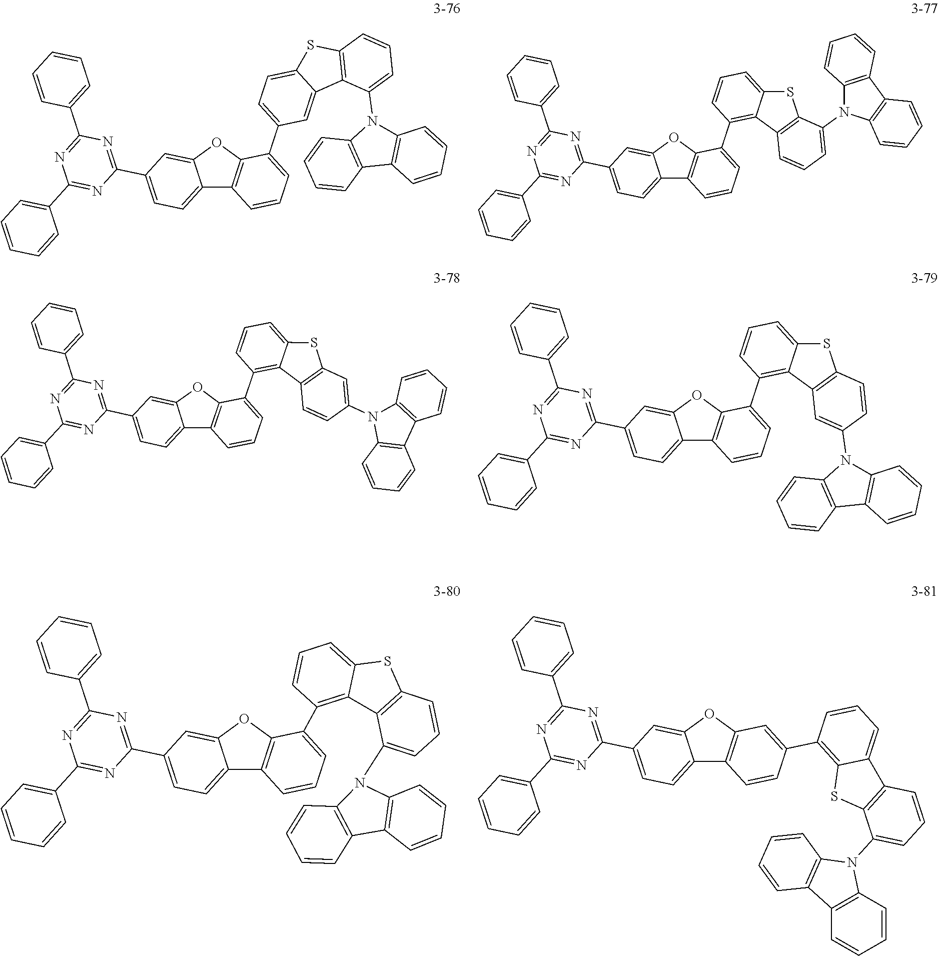

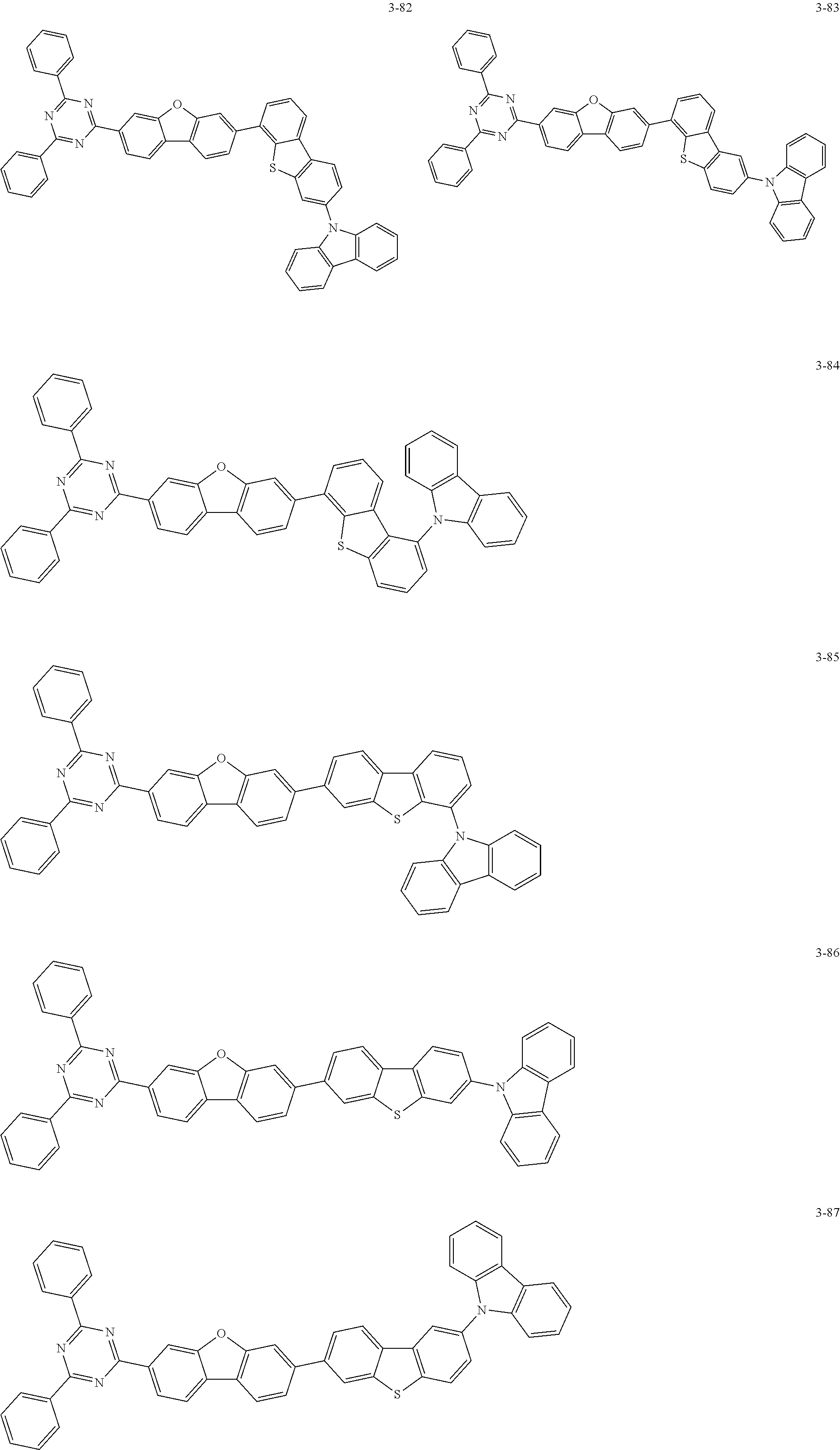

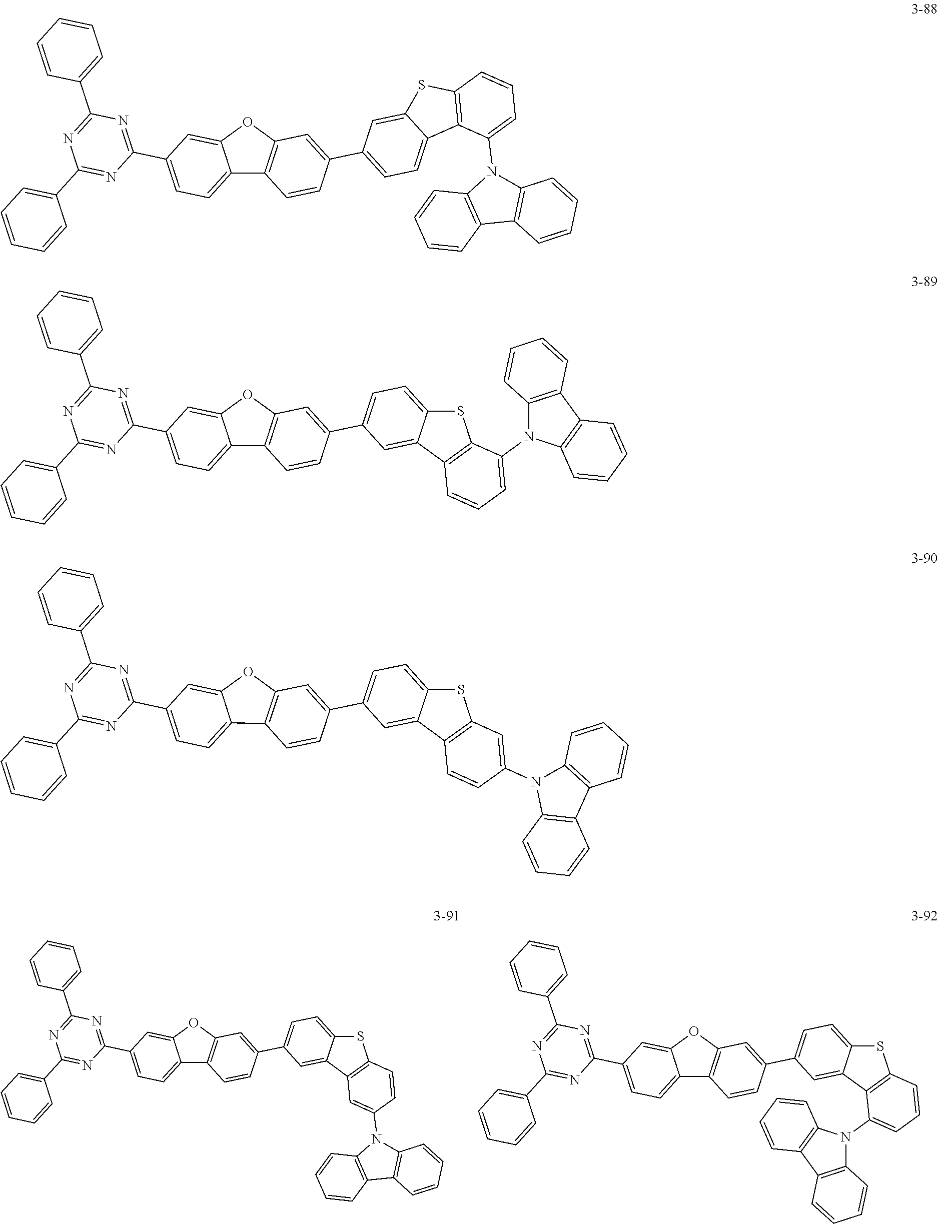

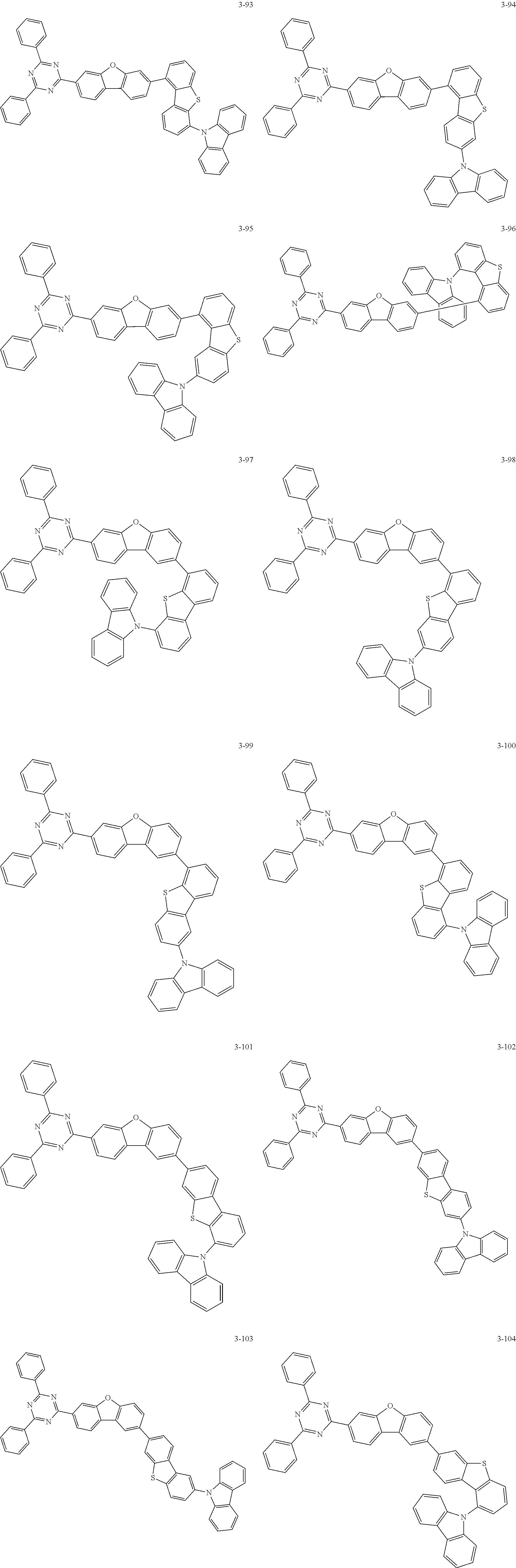

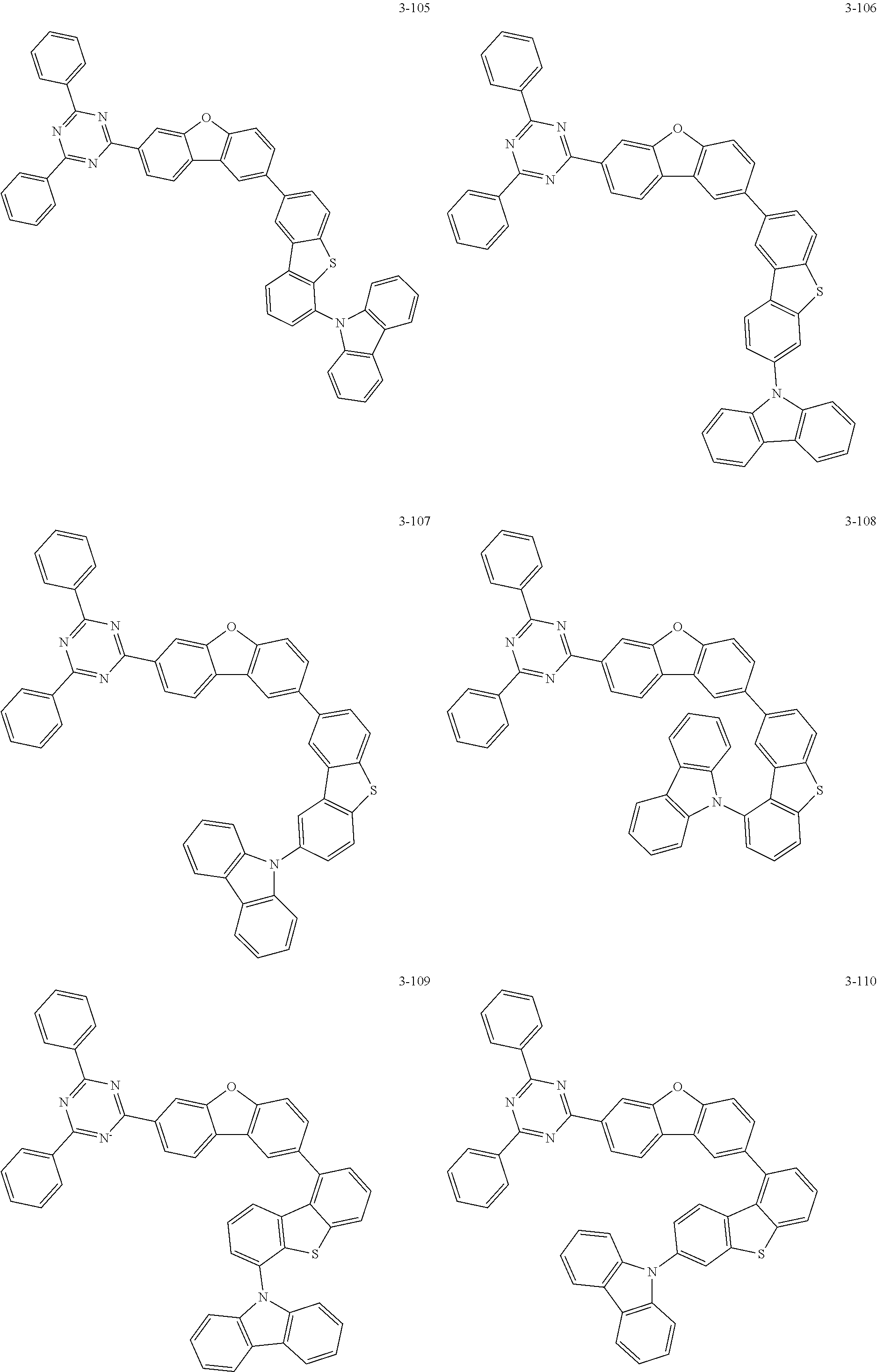

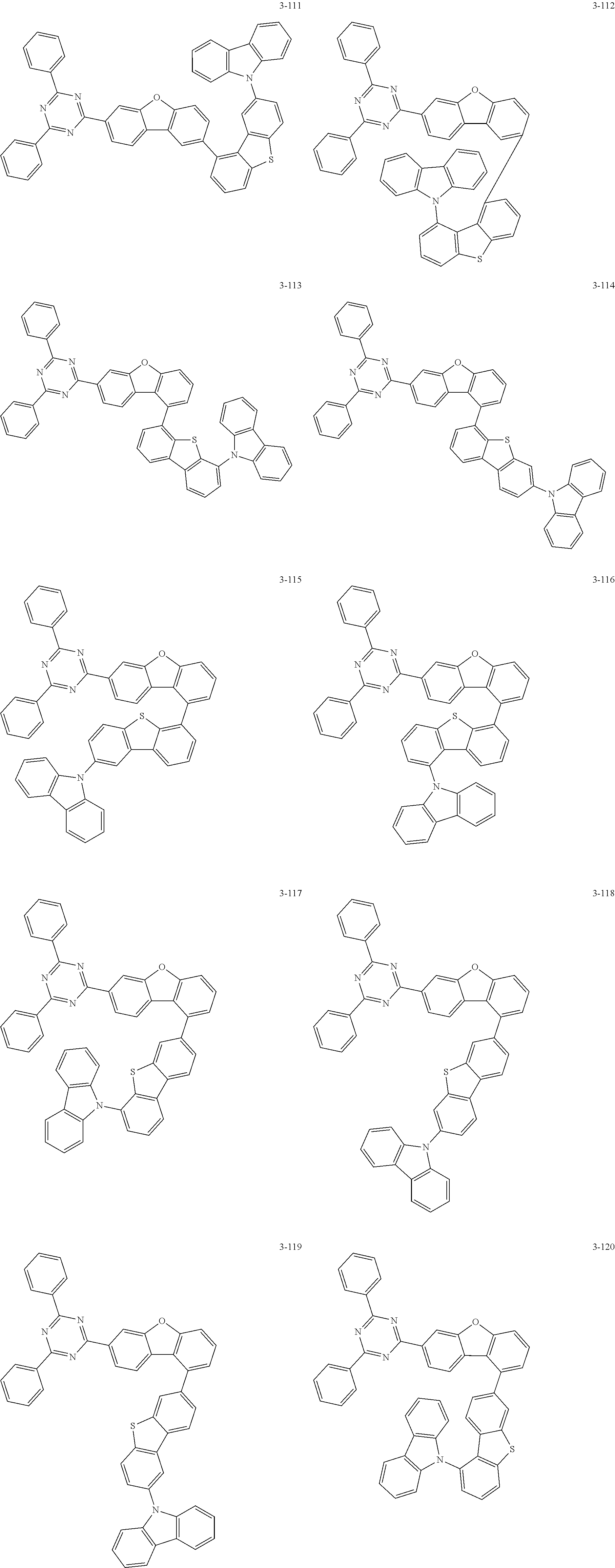

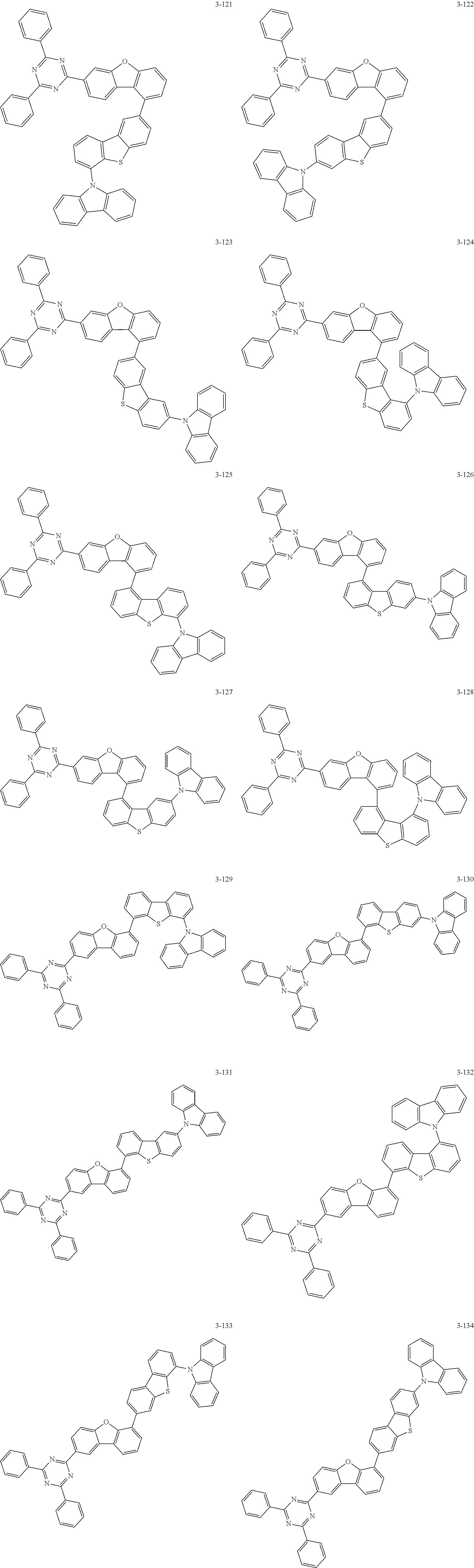

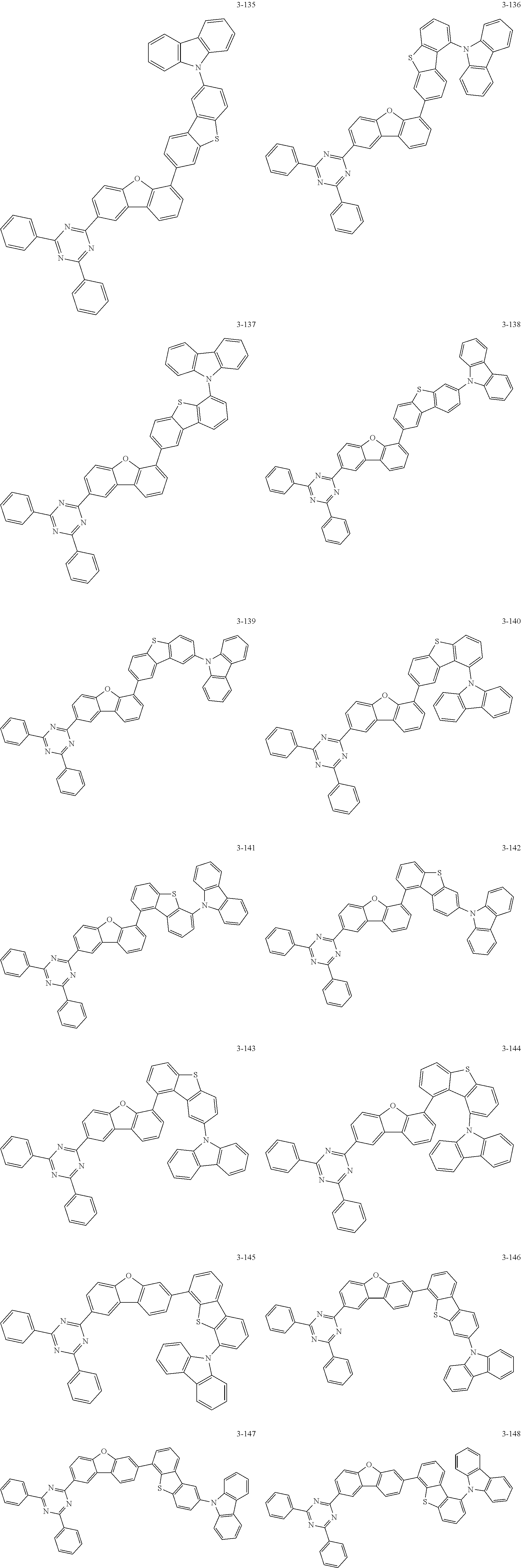

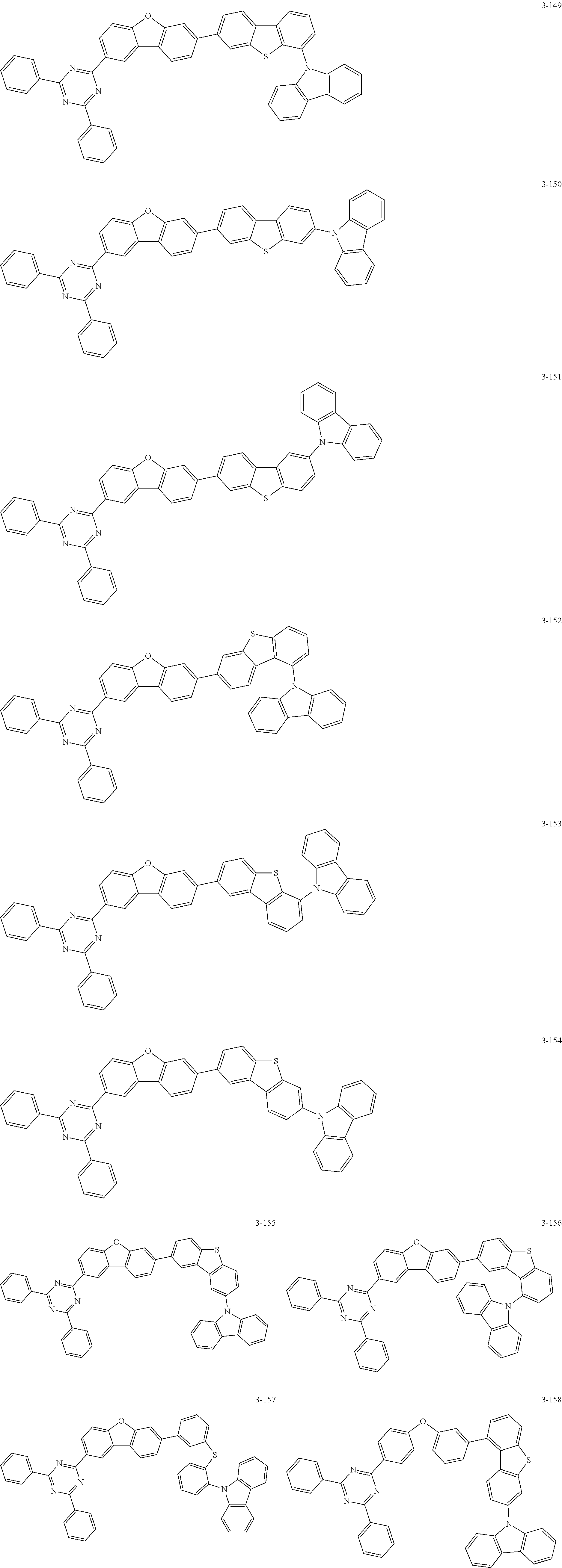

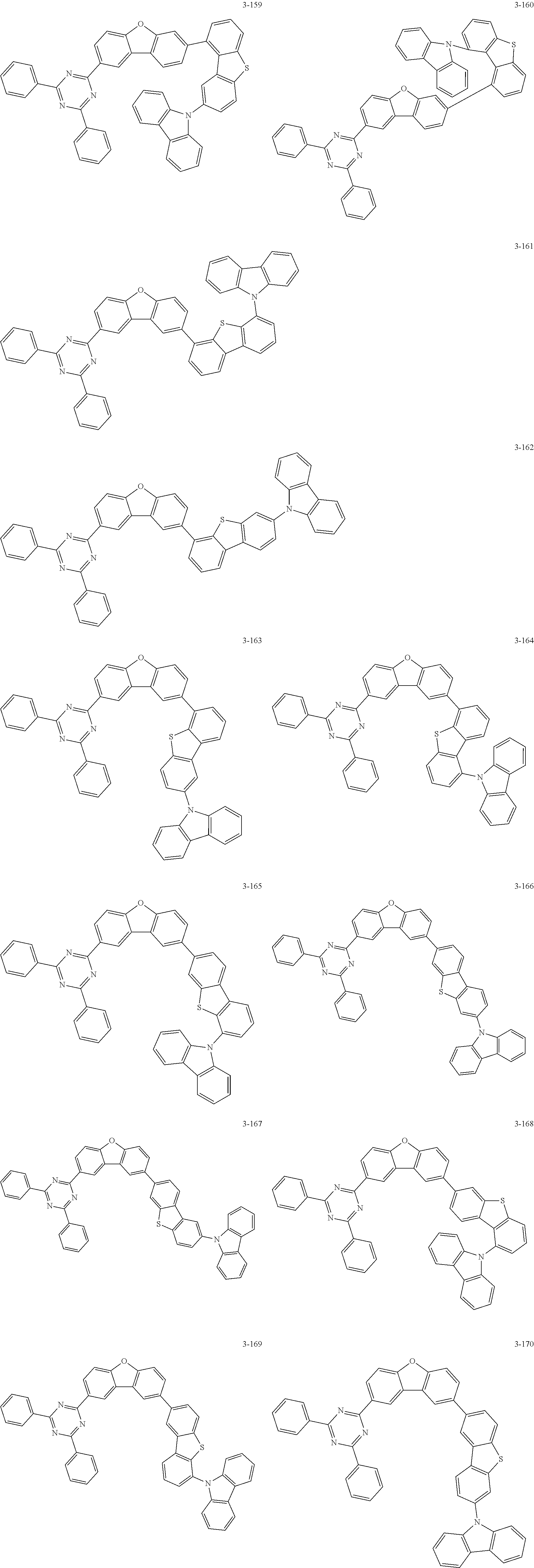

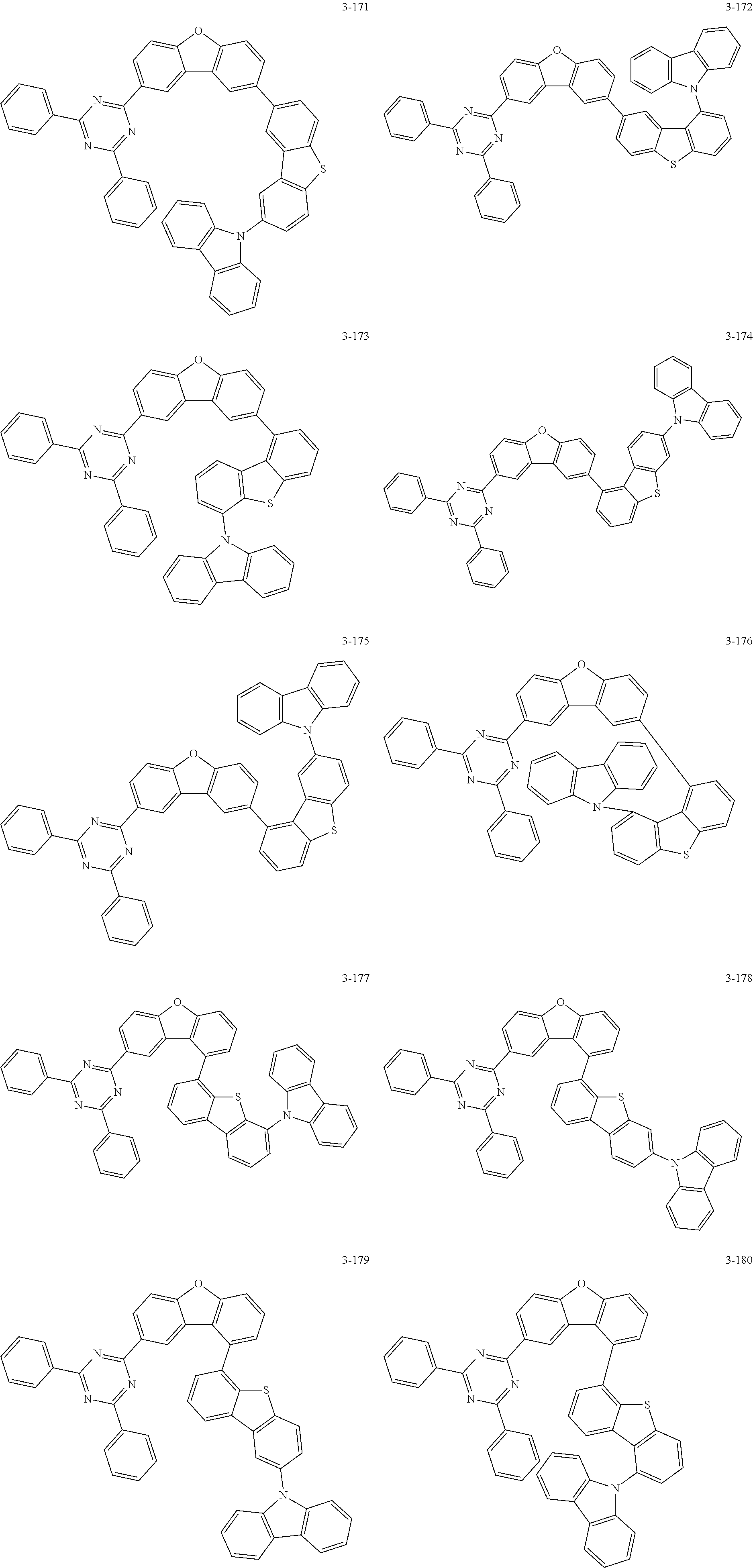

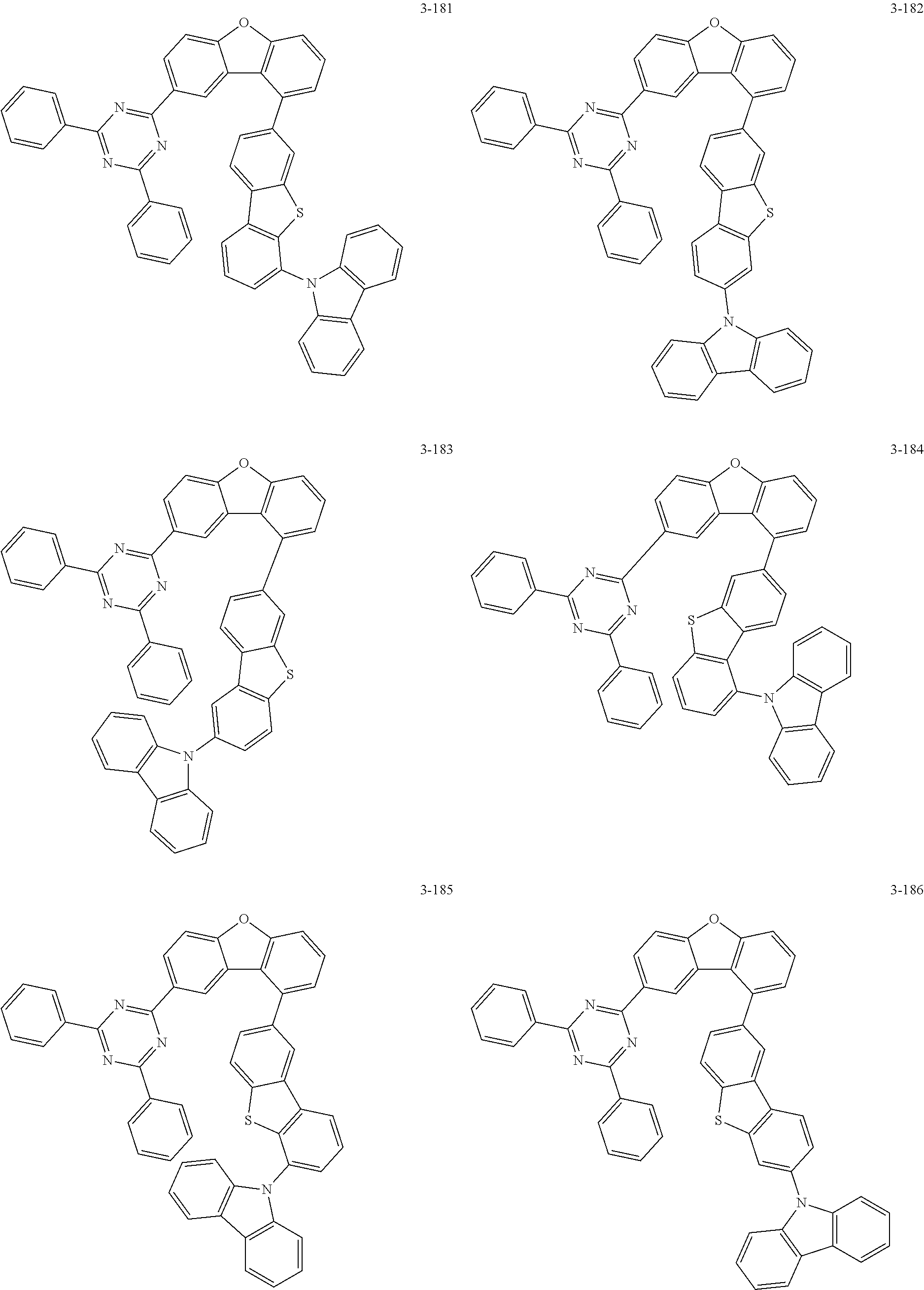

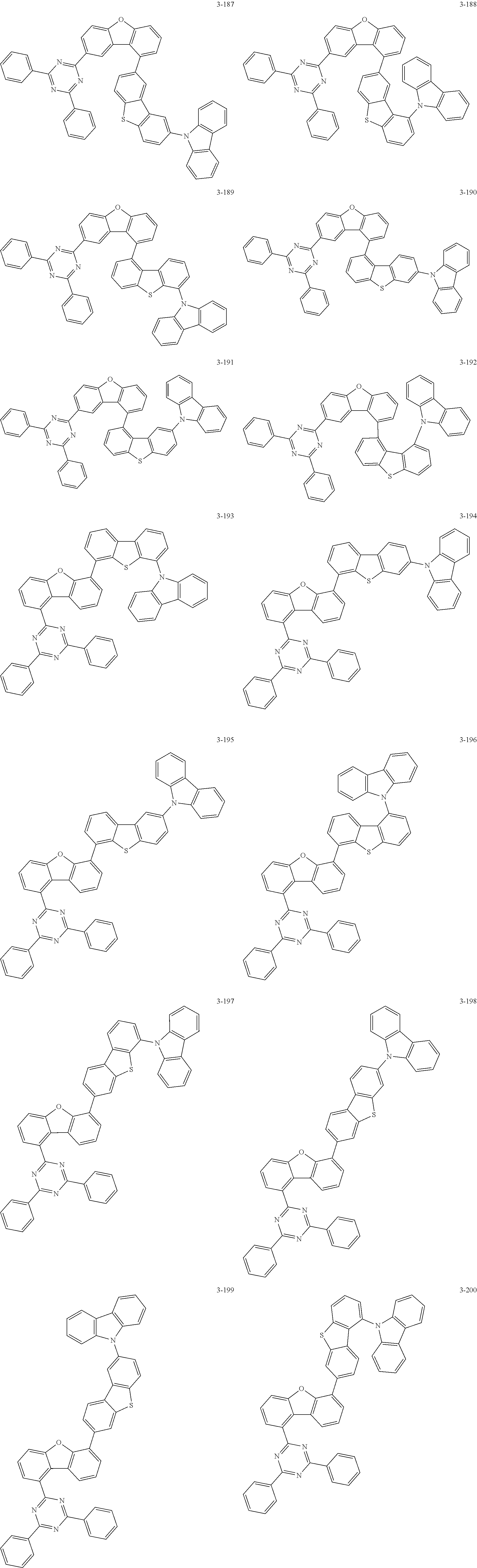

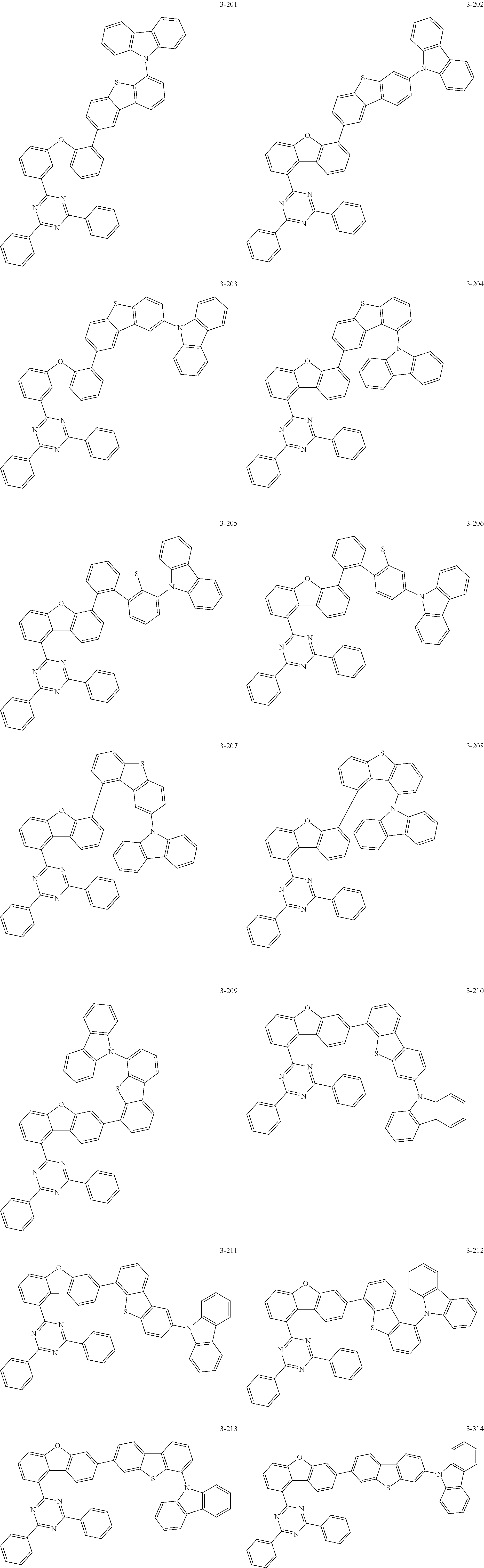

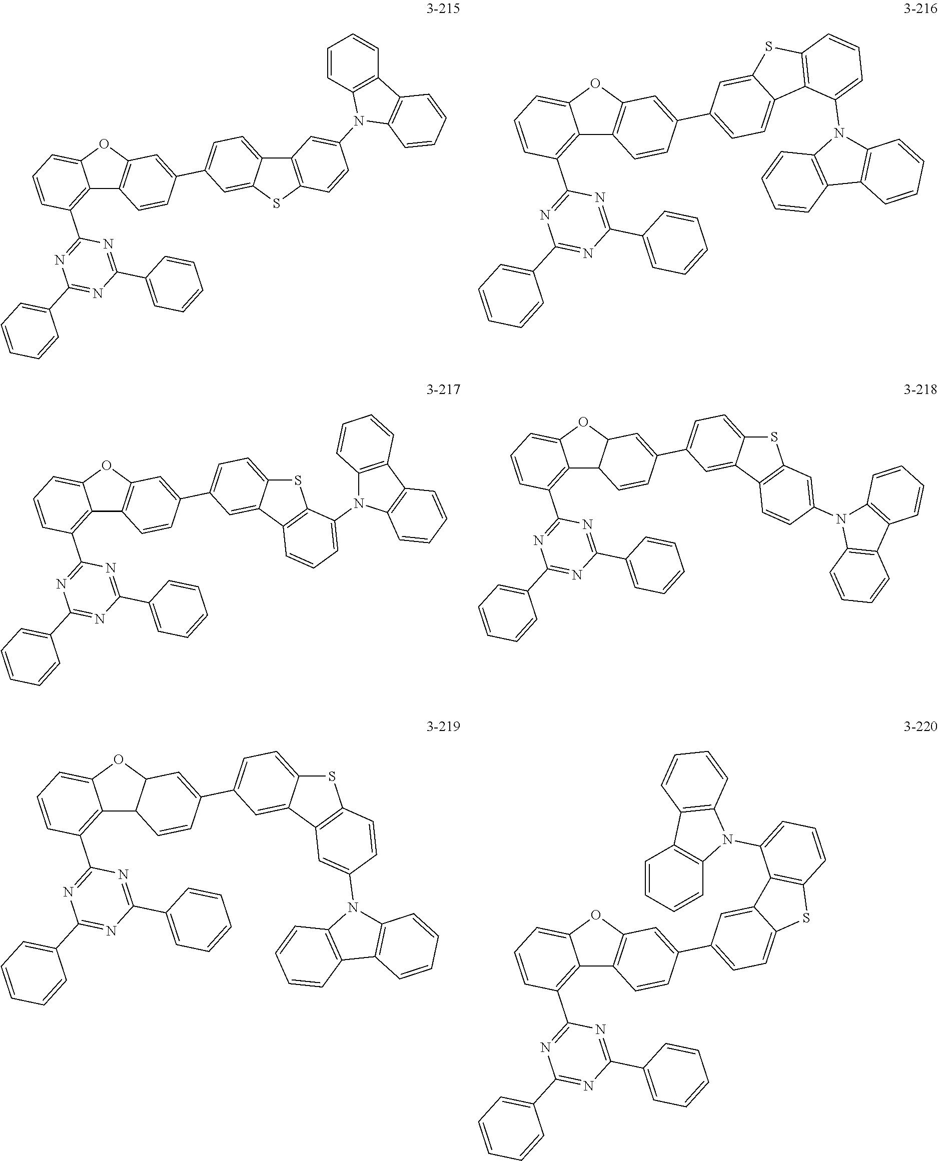

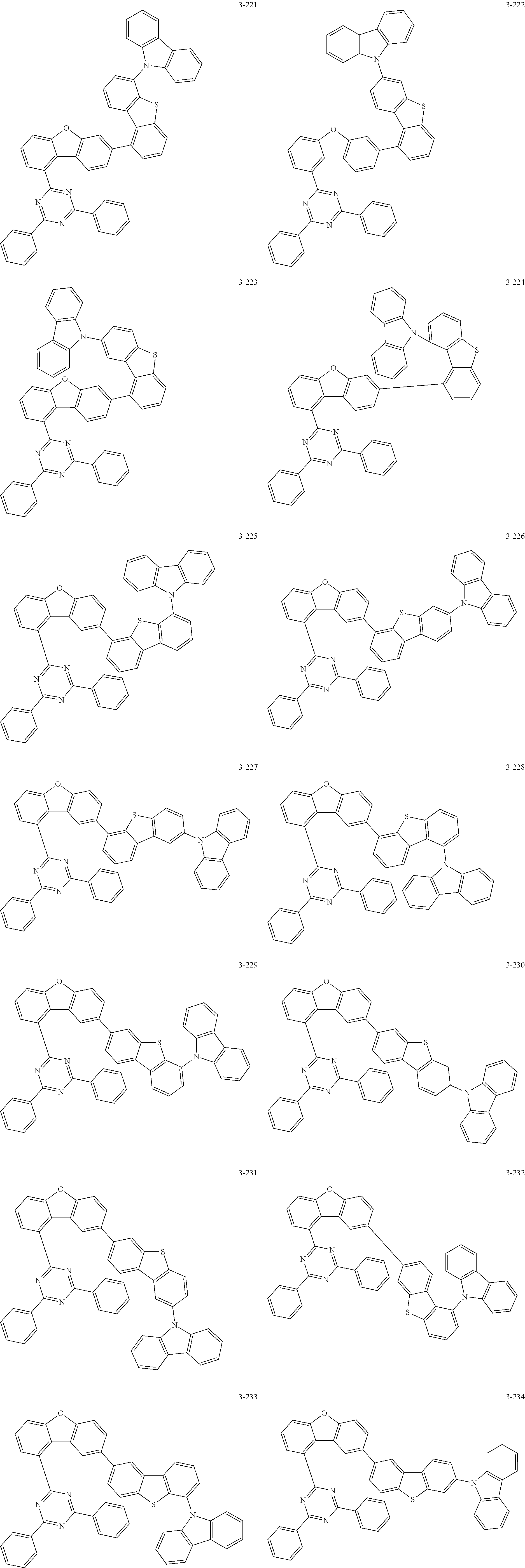

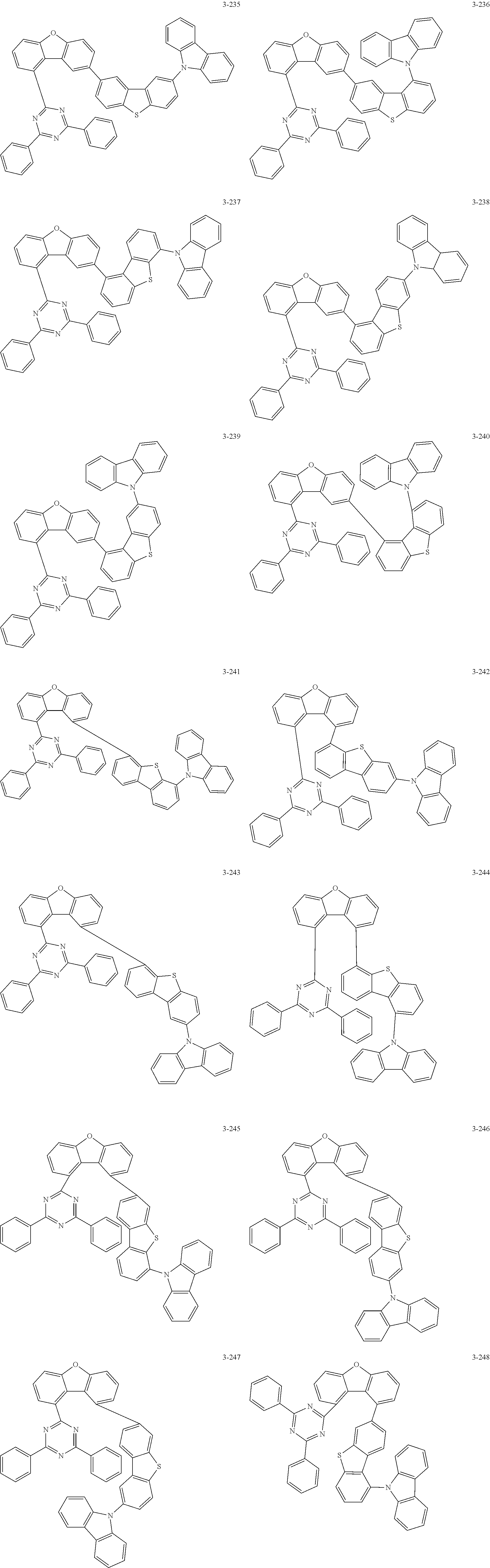

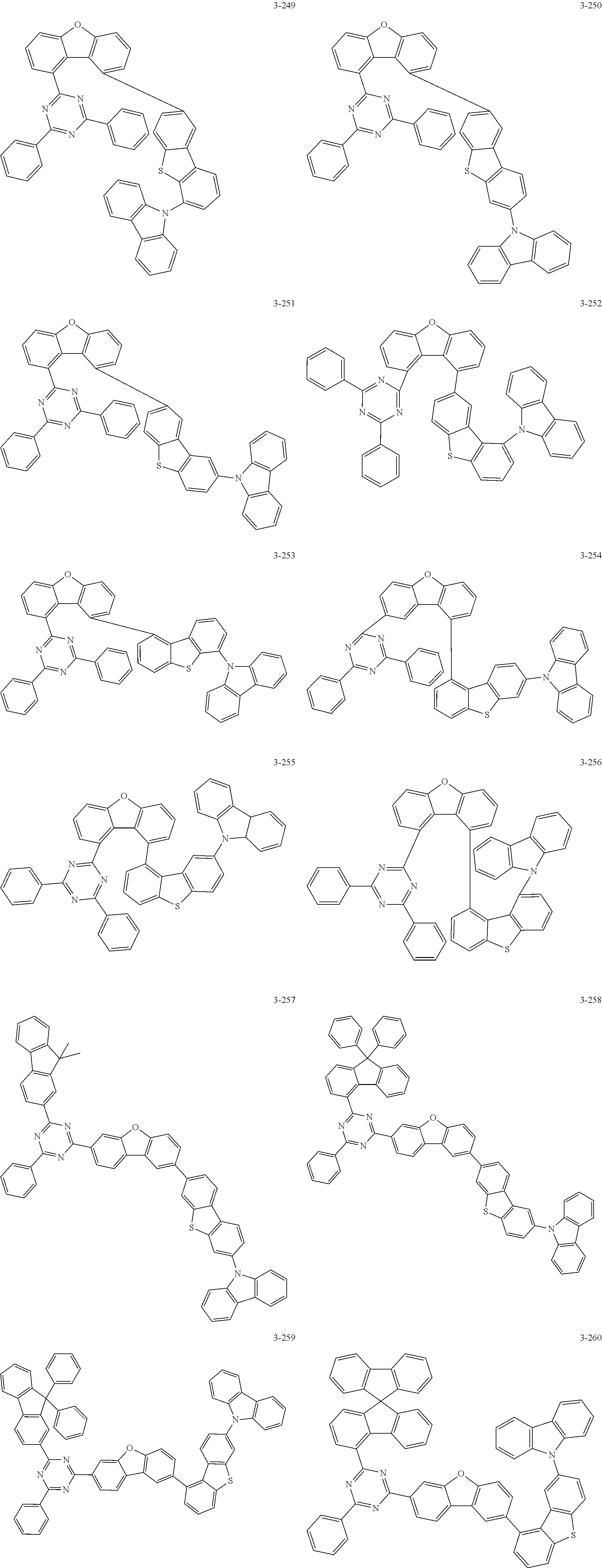

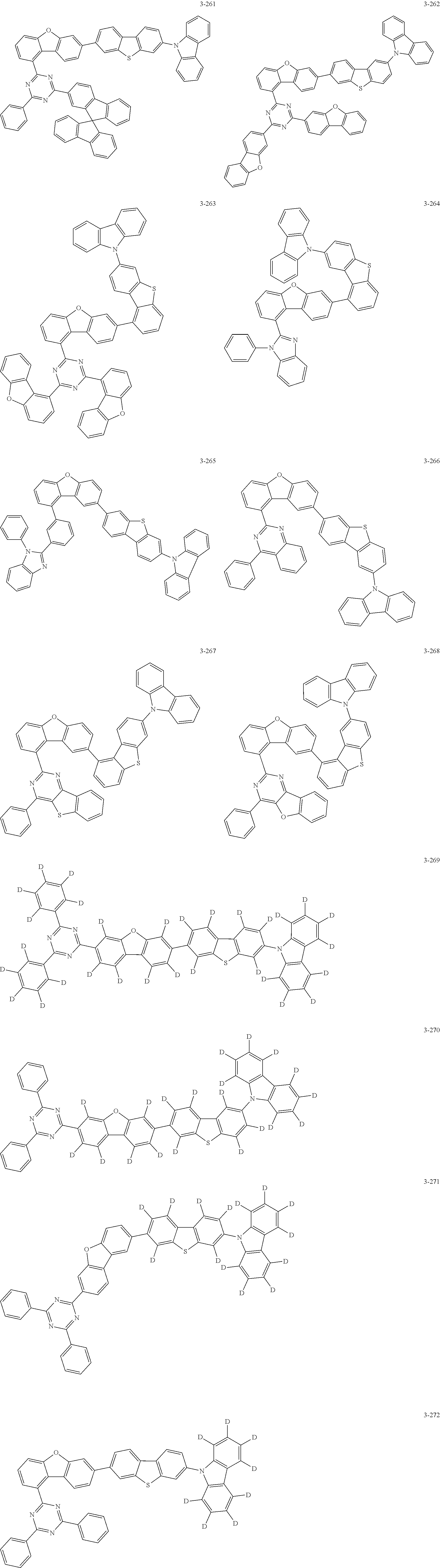

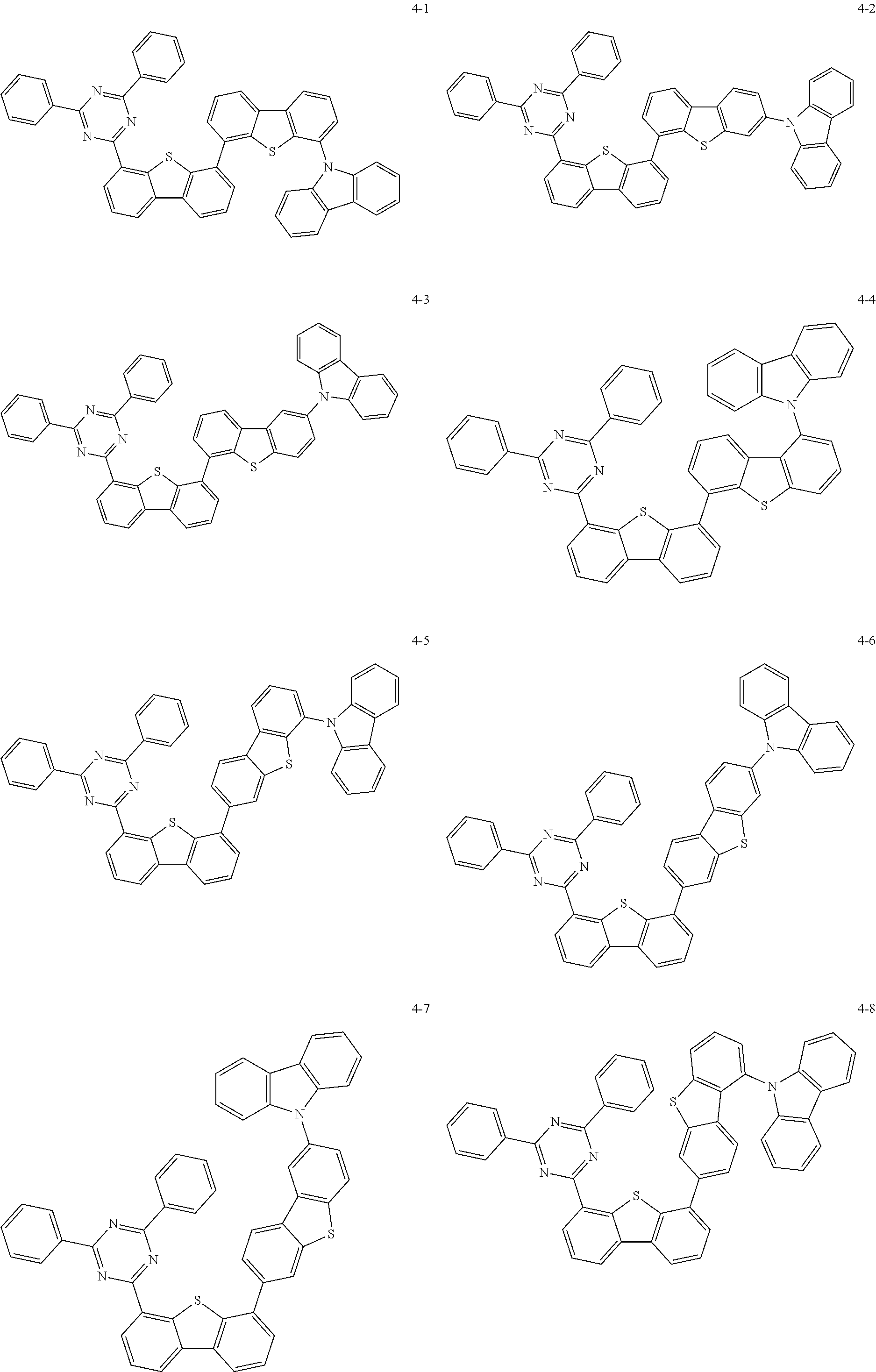

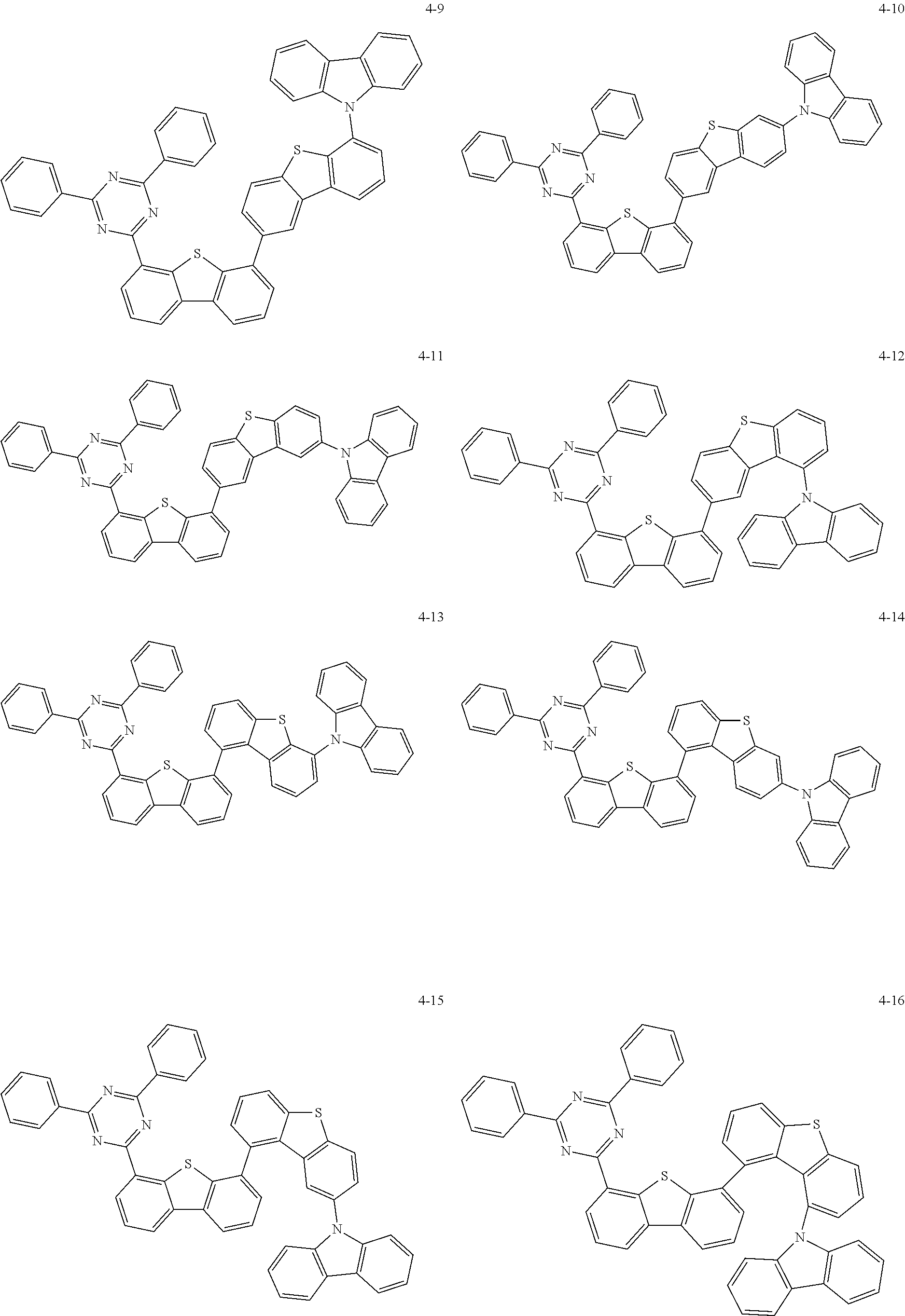

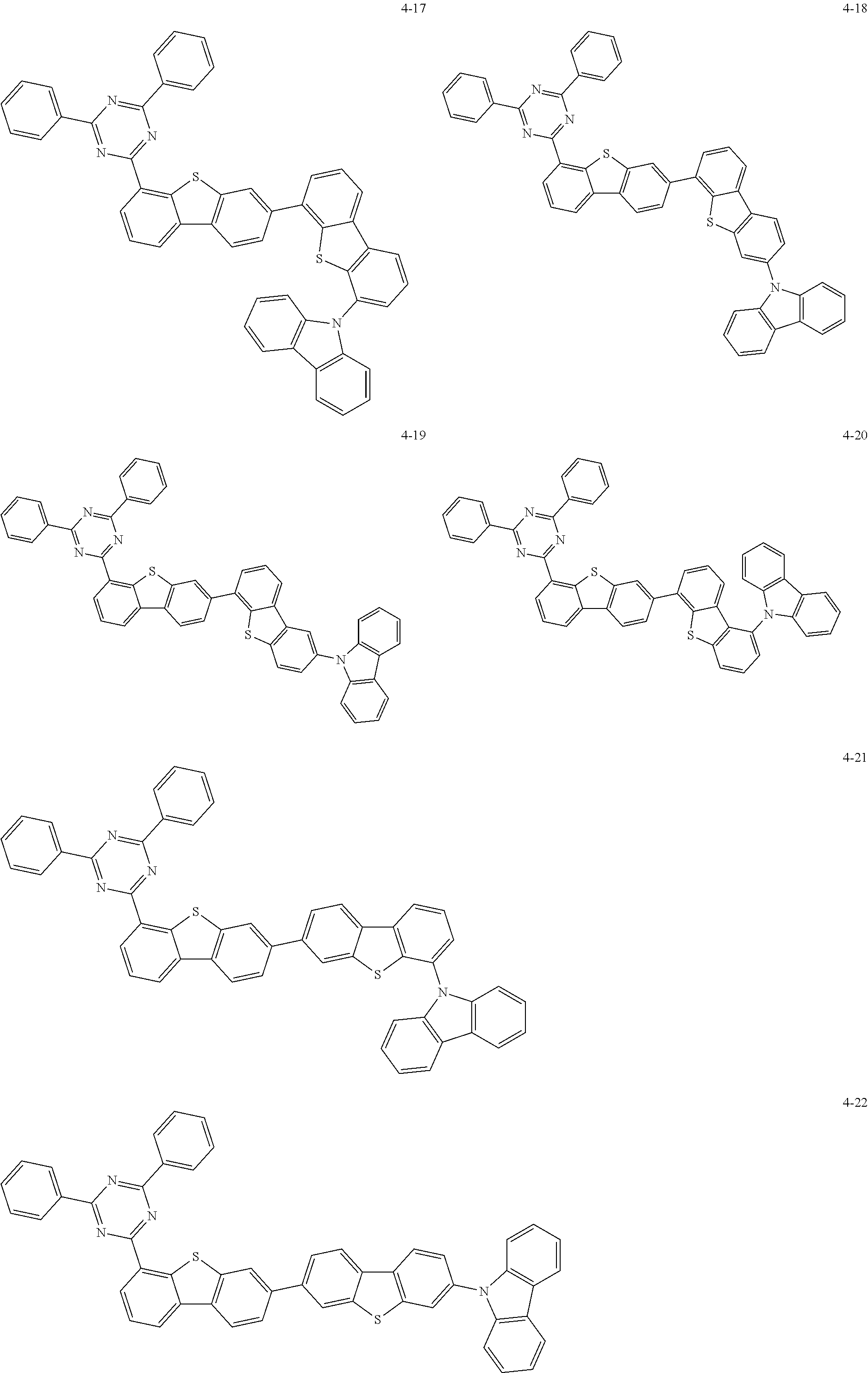

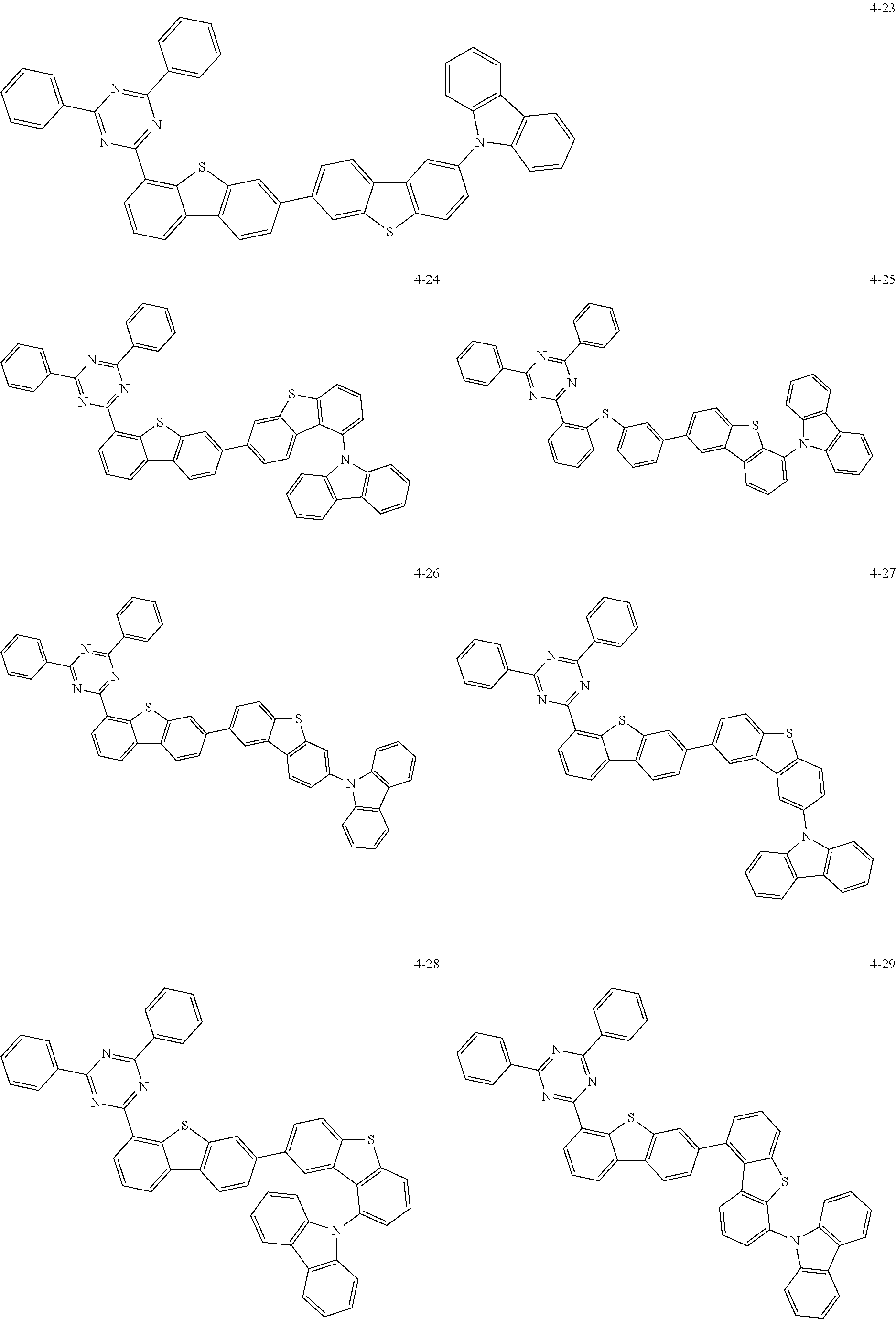

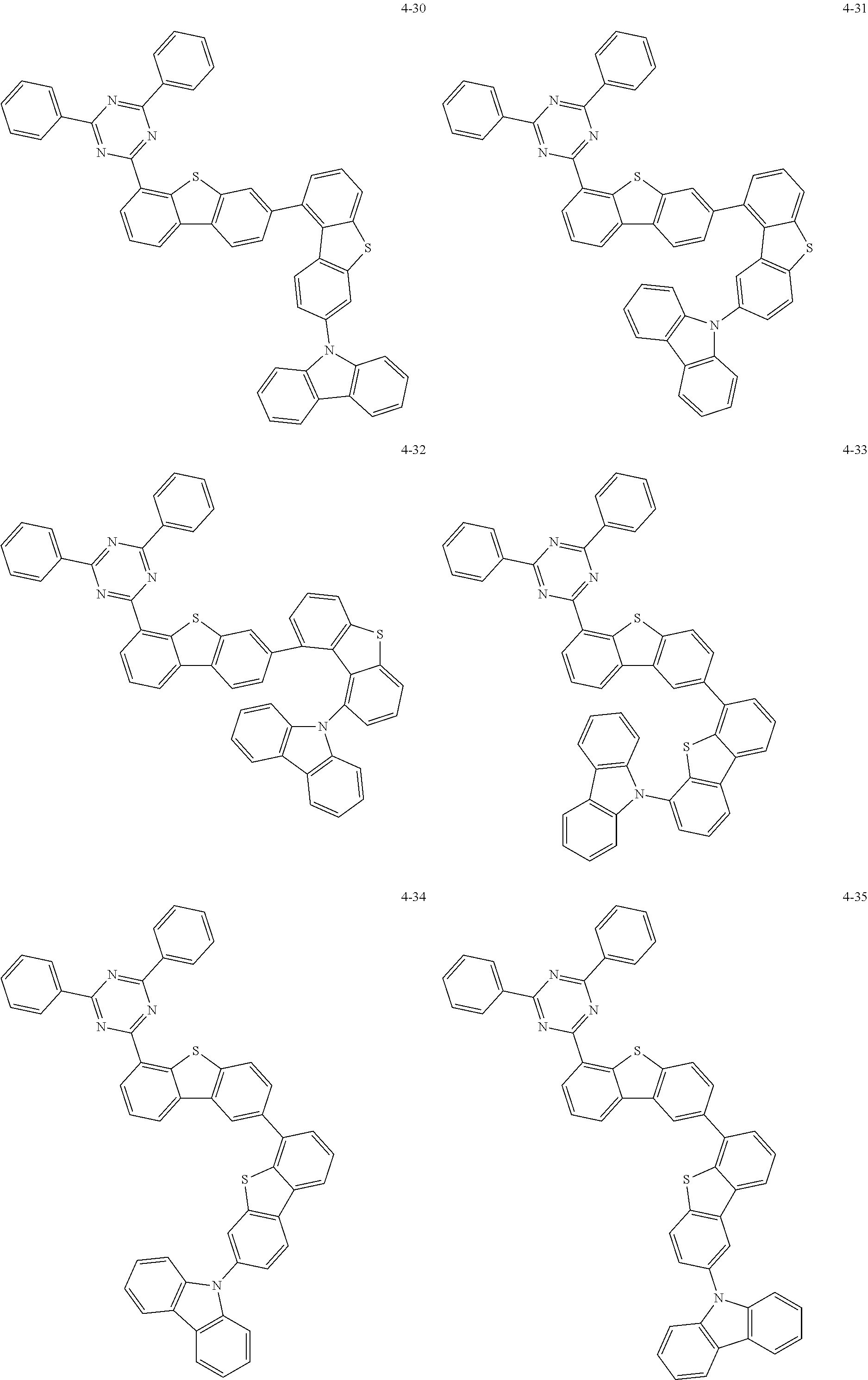

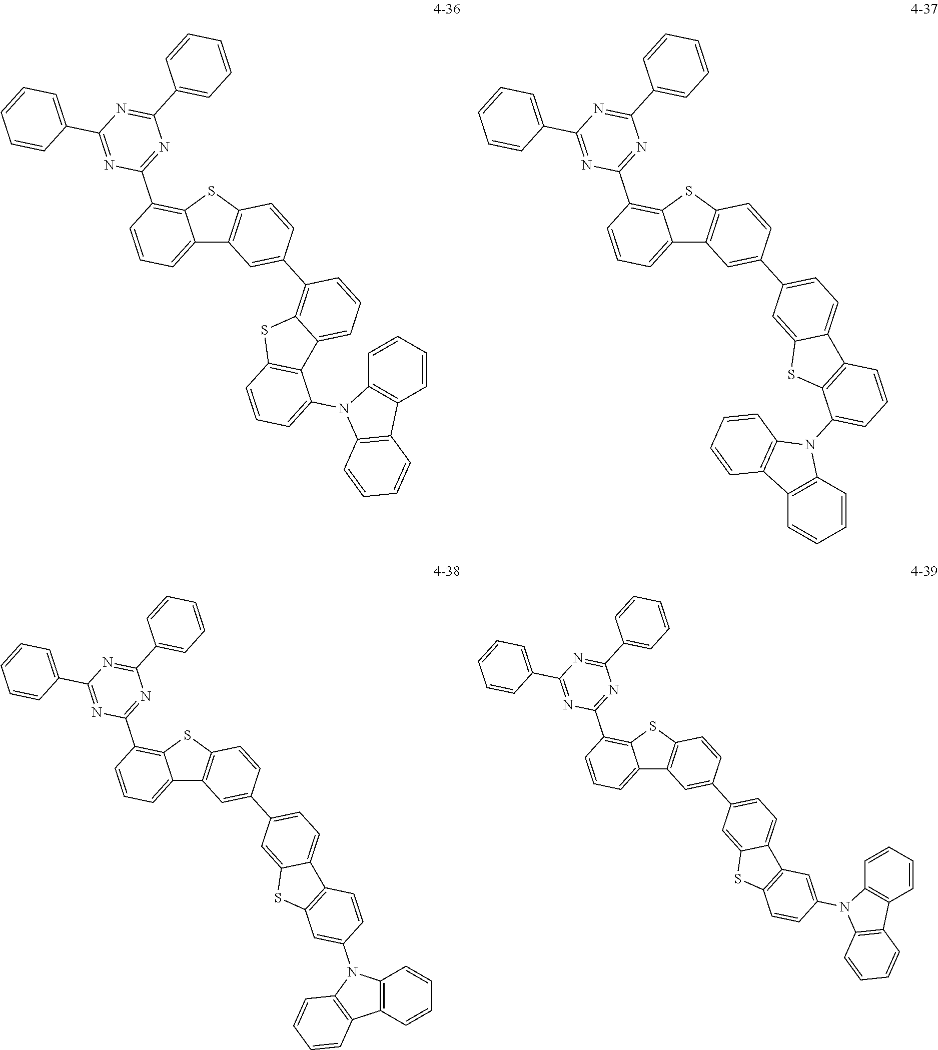 [SMILES: c1ccc(-c2nc(-c3ccccc3)nc(-c3cccc4c3sc3ccc(-c5cccc6c5sc5cccc(-n7c8ccccc8c8ccccc87)c56)cc34)n2)cc1 ; c1ccc(-c2nc(-c3ccccc3)nc(-c3cccc4c3sc3ccc(-c5ccc6c(c5)sc5c(-n7c8ccccc8c8ccccc87)cccc56)cc34)n2)cc1 ; c1ccc(-c2nc(-c3ccccc3)nc(-c3cccc4c3sc3ccc(-c5ccc6c(c5)sc5cc(-n7c8ccccc8c8ccccc87)ccc56)cc34)n2)cc1 ; c1ccc(-c2nc(-c3ccccc3)nc(-c3cccc4c3sc3ccc(-c5ccc6c(c5)sc5ccc(-n7c8ccccc8c8ccccc87)cc56)cc34)n2)cc1]

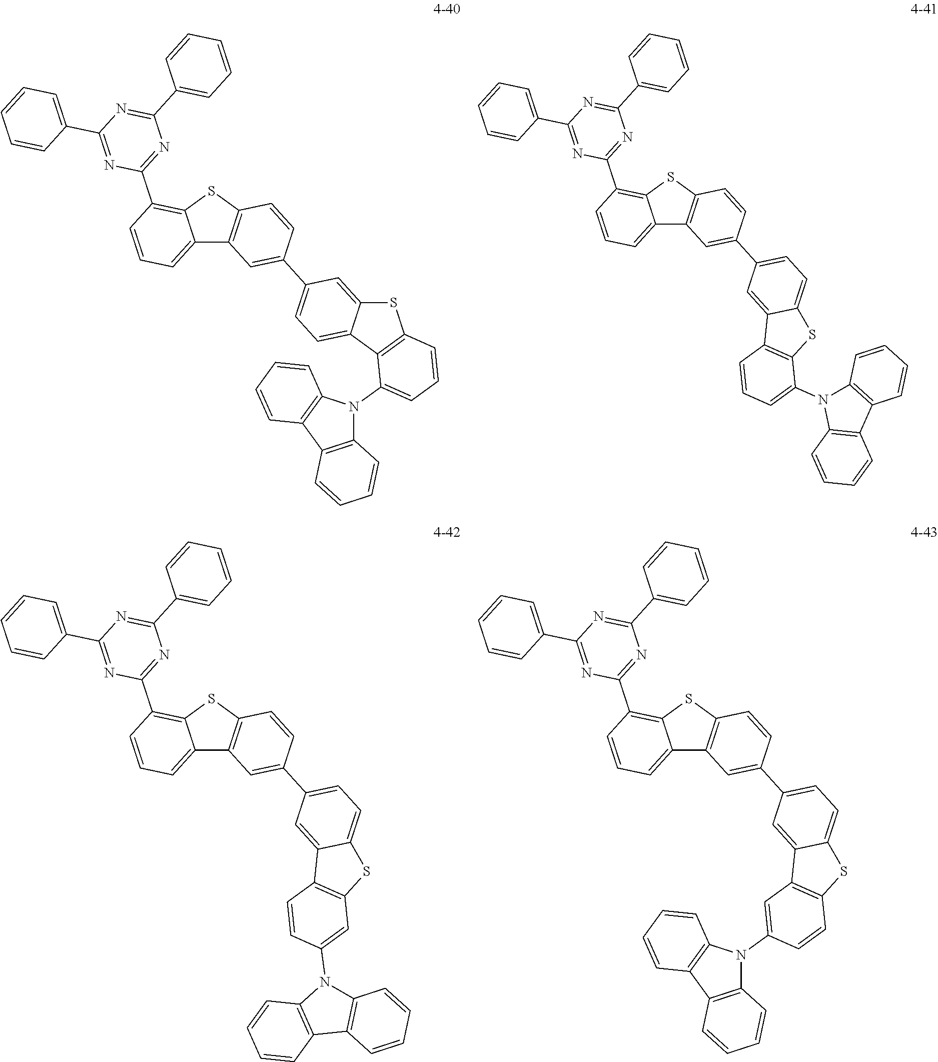 [SMILES: c1ccc(-c2nc(-c3ccccc3)nc(-c3cccc4c3sc3ccc(-c5ccc6c(c5)sc5cccc(-n7c8ccccc8c8ccccc87)c56)cc34)n2)cc1 ; c1ccc(-c2nc(-c3ccccc3)nc(-c3cccc4c3sc3ccc(-c5ccc6sc7c(-n8c9ccccc9c9ccccc98)cccc7c6c5)cc34)n2)cc1 ; c1ccc(-c2nc(-c3ccccc3)nc(-c3cccc4c3sc3ccc(-c5ccc6sc7cc(-n8c9ccccc9c9ccccc98)ccc7c6c5)cc34)n2)cc1 ; c1ccc(-c2nc(-c3ccccc3)nc(-c3cccc4c3sc3ccc(-c5ccc6sc7ccc(-n8c9ccccc9c9ccccc98)cc7c6c5)cc34)n2)cc1]

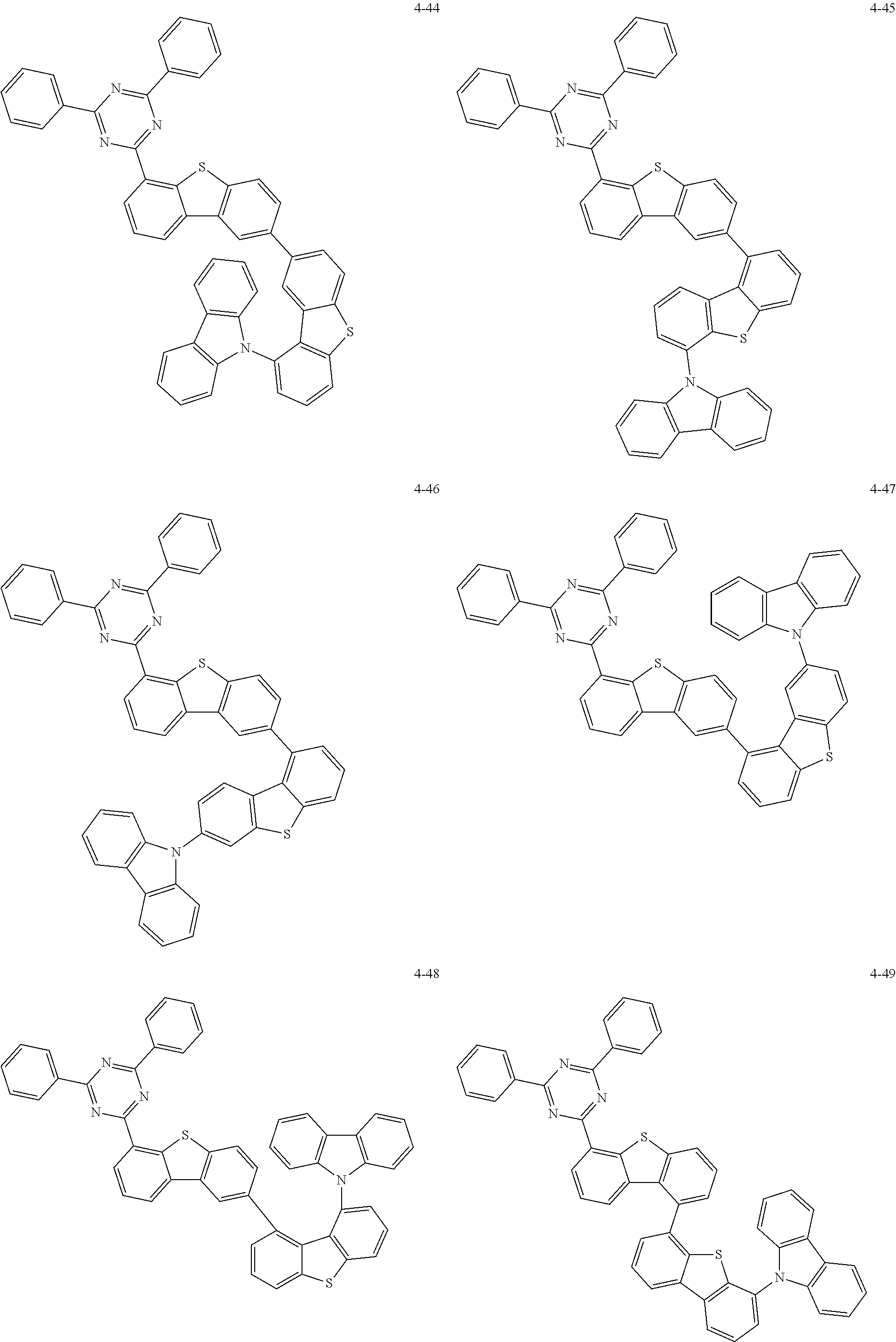

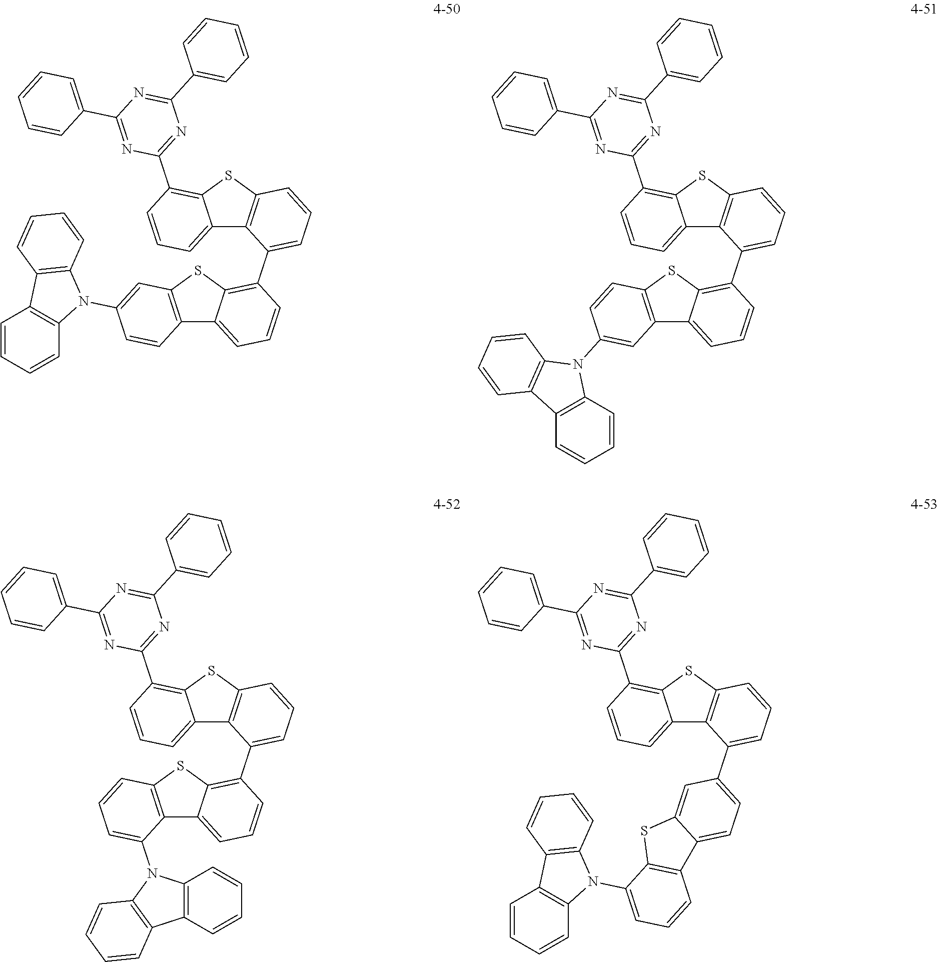 [SMILES: c1ccc(-c2nc(-c3ccccc3)nc(-c3cccc4c3sc3cccc(-c5cccc6c5sc5cc(-n7c8ccccc8c8ccccc87)ccc56)c34)n2)cc1 ; c1ccc(-c2nc(-c3ccccc3)nc(-c3cccc4c3sc3cccc(-c5cccc6c5sc5ccc(-n7c8ccccc8c8ccccc87)cc56)c34)n2)cc1 ; c1ccc(-c2nc(-c3ccccc3)nc(-c3cccc4c3sc3cccc(-c5cccc6c5sc5cccc(-n7c8ccccc8c8ccccc87)c56)c34)n2)cc1 ; c1ccc(-c2nc(-c3ccccc3)nc(-c3cccc4c3sc3cccc(-c5ccc6c(c5)sc5c(-n7c8ccccc8c8ccccc87)cccc56)c34)n2)cc1]

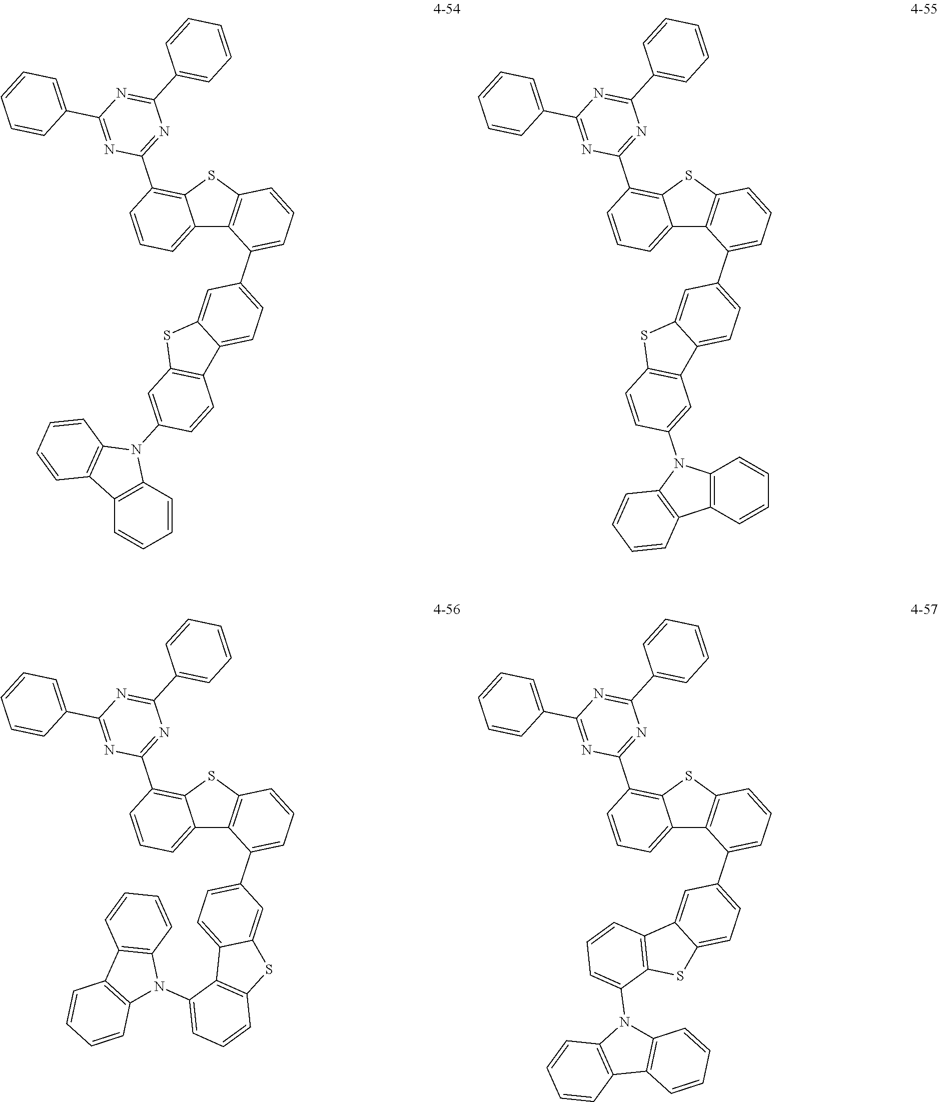 [SMILES: c1ccc(-c2nc(-c3ccccc3)nc(-c3cccc4c3sc3cccc(-c5ccc6c(c5)sc5cc(-n7c8ccccc8c8ccccc87)ccc56)c34)n2)cc1 ; c1ccc(-c2nc(-c3ccccc3)nc(-c3cccc4c3sc3cccc(-c5ccc6c(c5)sc5ccc(-n7c8ccccc8c8ccccc87)cc56)c34)n2)cc1 ; c1ccc(-c2nc(-c3ccccc3)nc(-c3cccc4c3sc3cccc(-c5ccc6c(c5)sc5cccc(-n7c8ccccc8c8ccccc87)c56)c34)n2)cc1 ; c1ccc(-c2nc(-c3ccccc3)nc(-c3cccc4c3sc3cccc(-c5ccc6sc7c(-n8c9ccccc9c9ccccc98)cccc7c6c5)c34)n2)cc1]

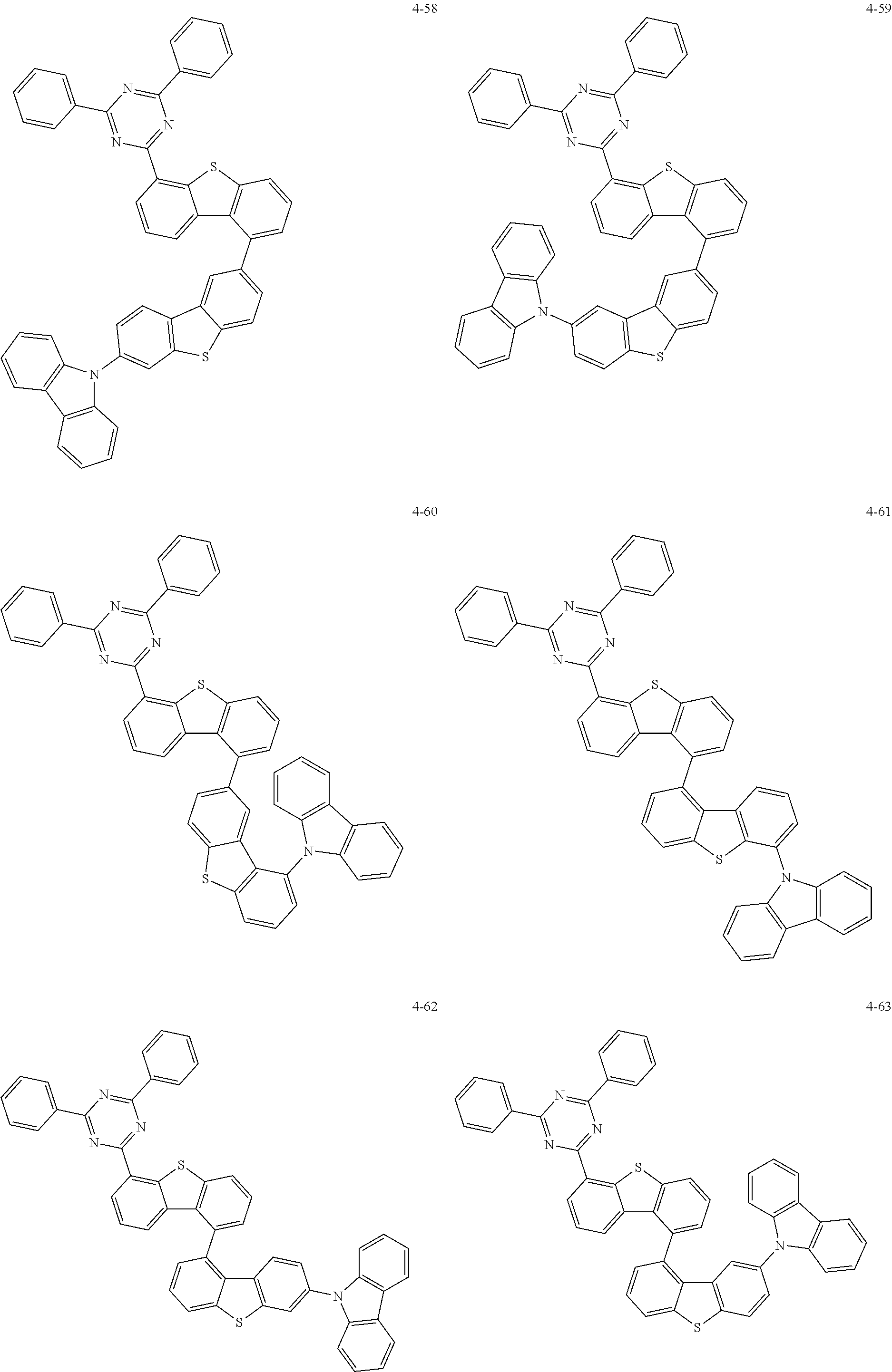

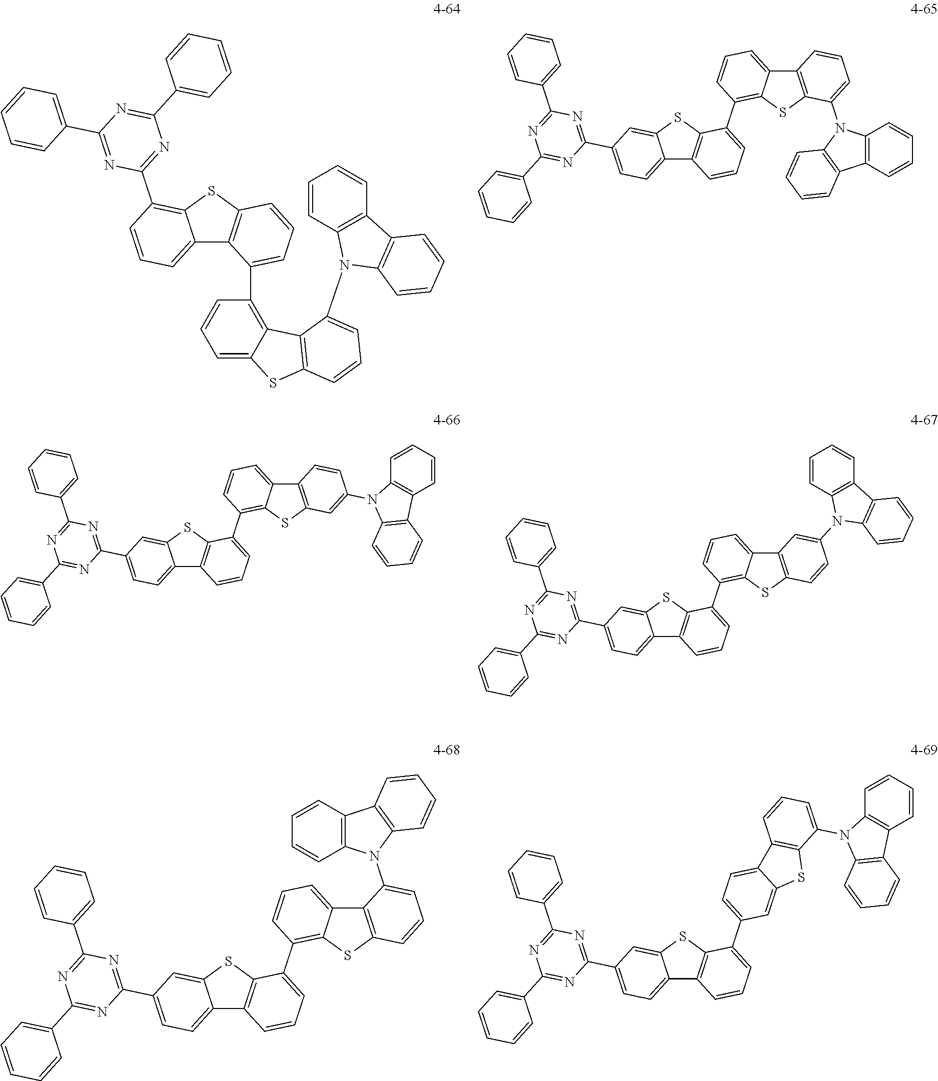

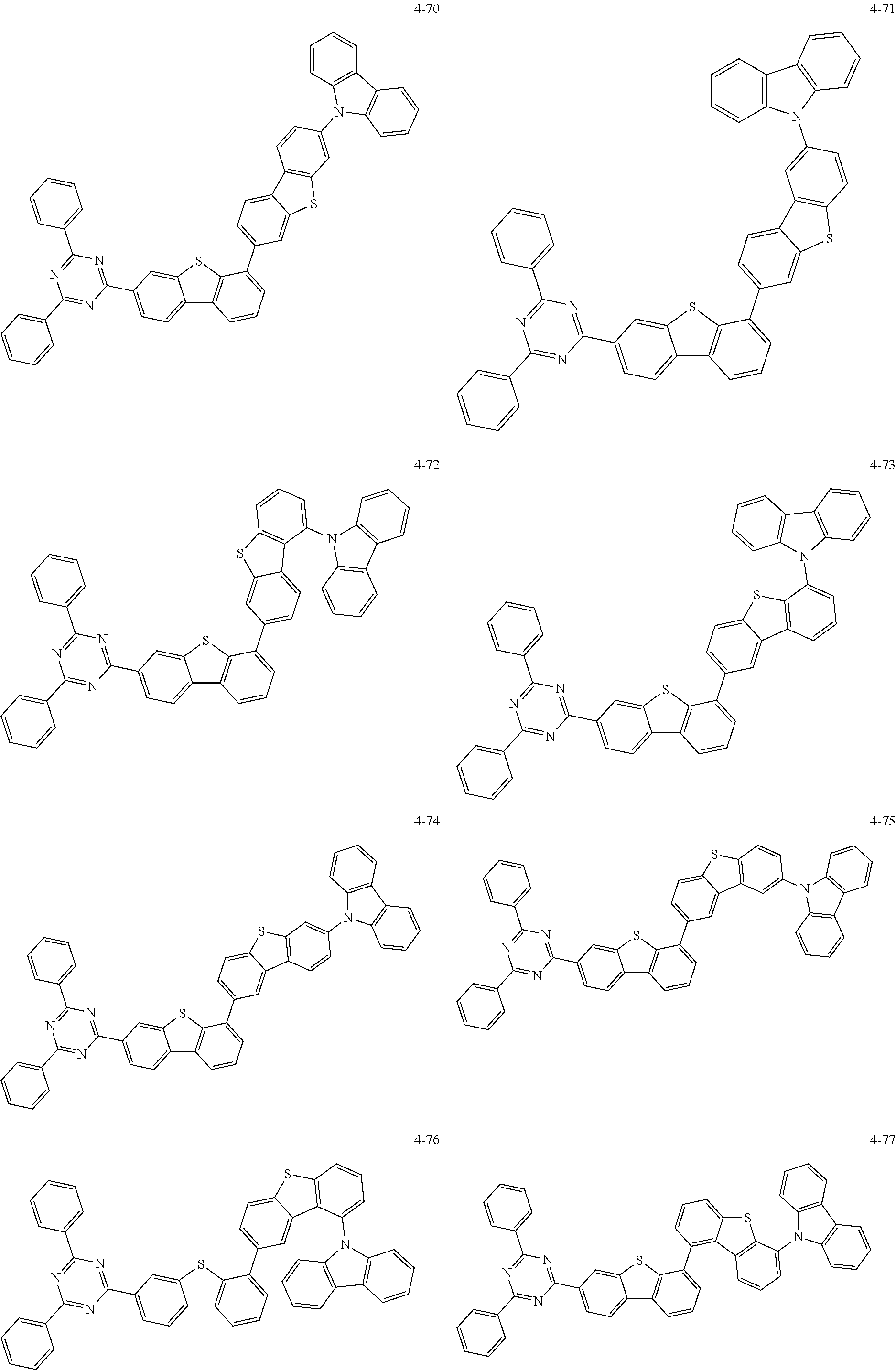

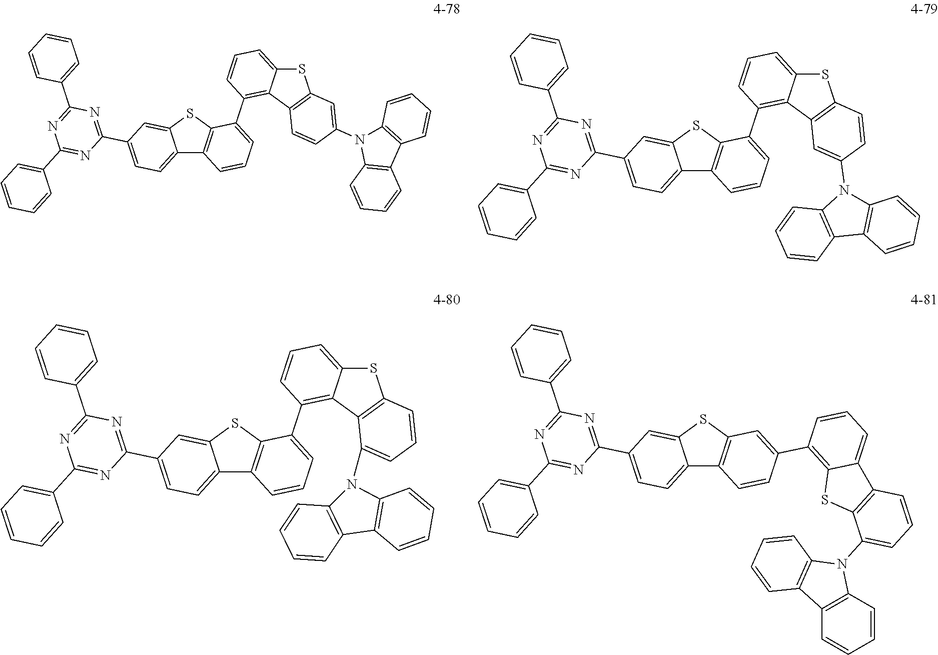 [SMILES: c1ccc(-c2nc(-c3ccccc3)nc(-c3ccc4c(c3)sc3c(-c5cccc6sc7cc(-n8c9ccccc9c9ccccc98)ccc7c56)cccc34)n2)cc1 ; c1ccc(-c2nc(-c3ccccc3)nc(-c3ccc4c(c3)sc3c(-c5cccc6sc7ccc(-n8c9ccccc9c9ccccc98)cc7c56)cccc34)n2)cc1 ; c1ccc(-c2nc(-c3ccccc3)nc(-c3ccc4c(c3)sc3c(-c5cccc6sc7cccc(-n8c9ccccc9c9ccccc98)c7c56)cccc34)n2)cc1 ; c1ccc(-c2nc(-c3ccccc3)nc(-c3ccc4c(c3)sc3cc(-c5cccc6c5sc5c(-n7c8ccccc8c8ccccc87)cccc56)ccc34)n2)cc1]

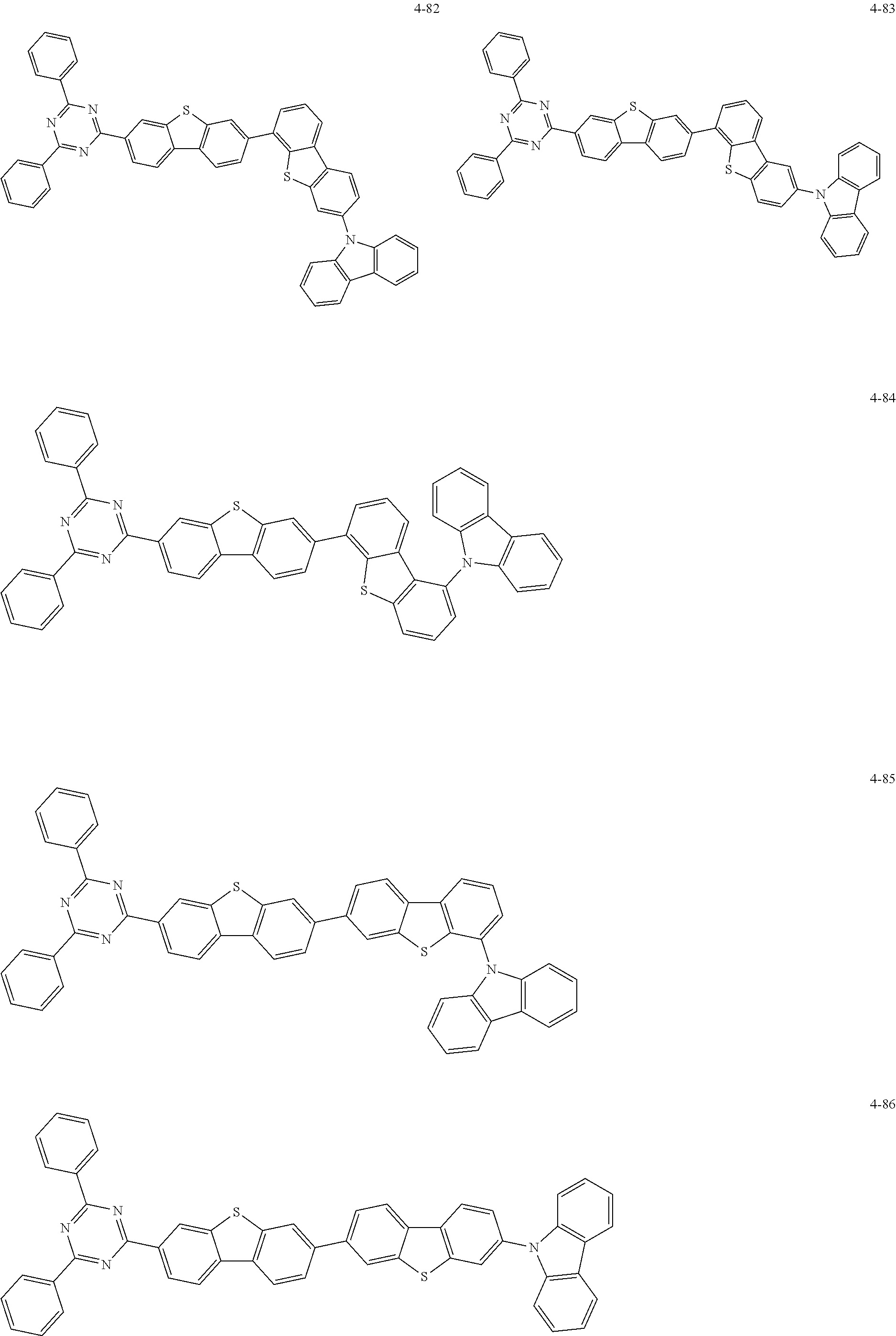

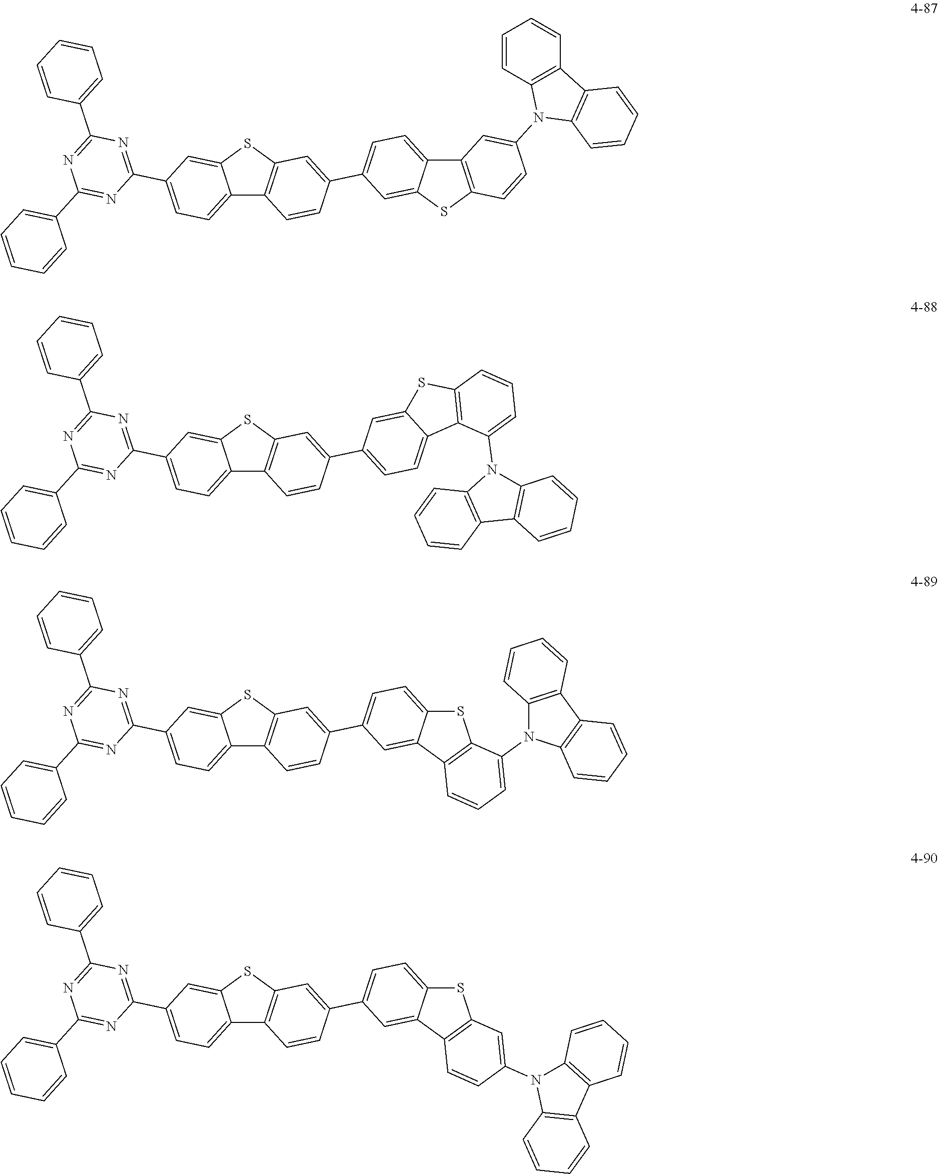

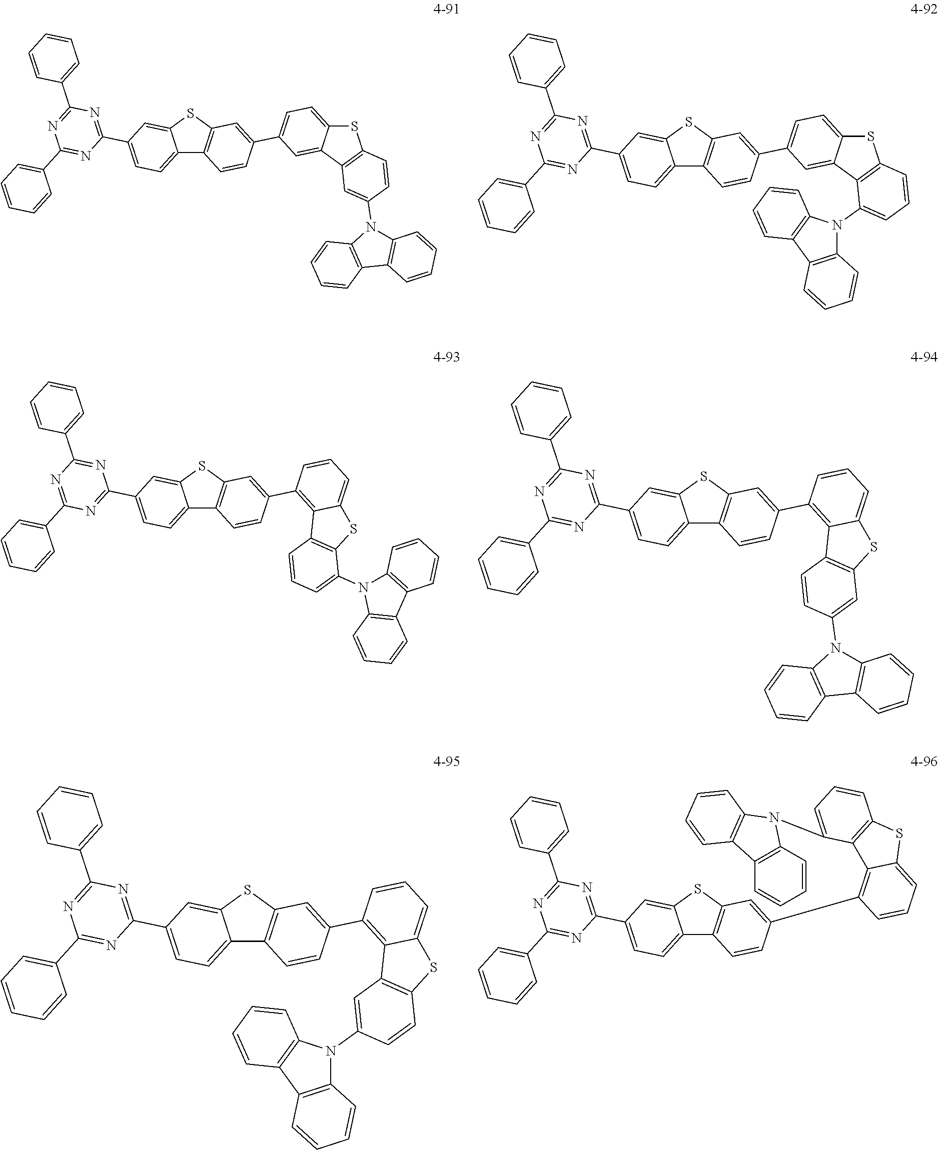

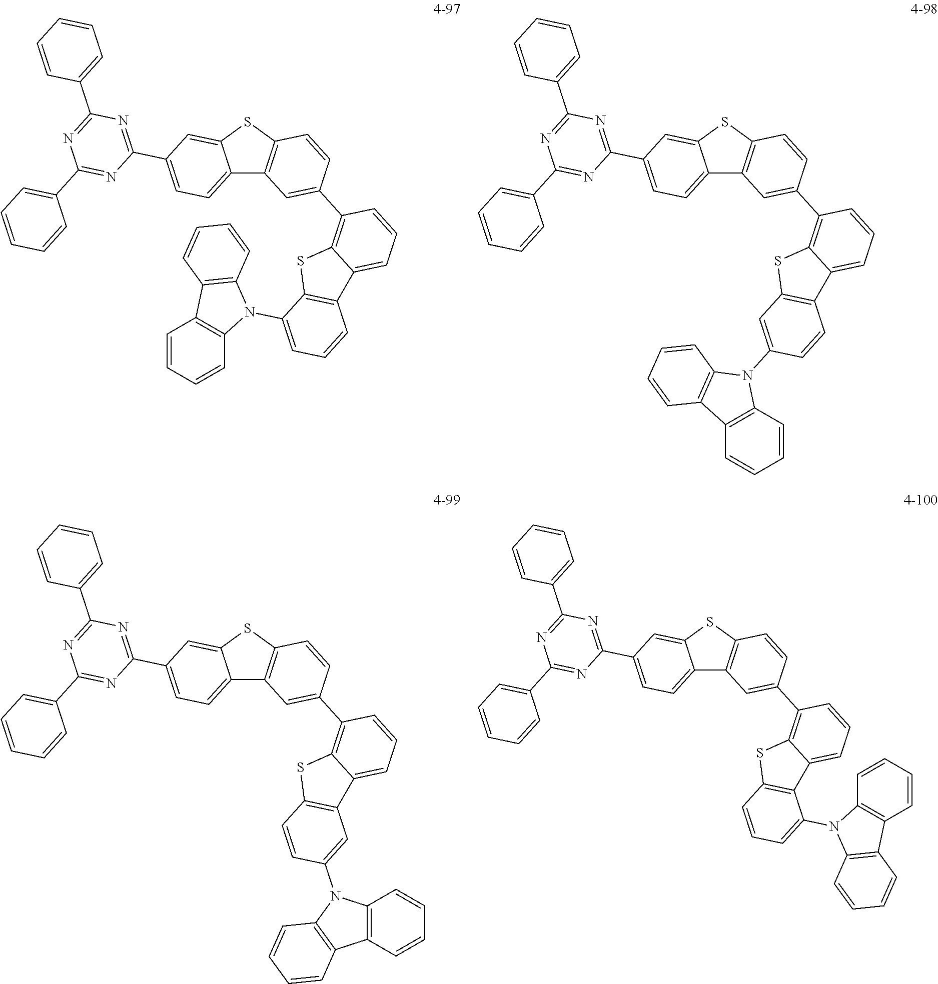 [SMILES: c1ccc(-c2nc(-c3ccccc3)nc(-c3ccc4c(c3)sc3ccc(-c5cccc6c5sc5c(-n7c8ccccc8c8ccccc87)cccc56)cc34)n2)cc1 ; c1ccc(-c2nc(-c3ccccc3)nc(-c3ccc4c(c3)sc3ccc(-c5cccc6c5sc5cc(-n7c8ccccc8c8ccccc87)ccc56)cc34)n2)cc1 ; c1ccc(-c2nc(-c3ccccc3)nc(-c3ccc4c(c3)sc3ccc(-c5cccc6c5sc5ccc(-n7c8ccccc8c8ccccc87)cc56)cc34)n2)cc1 ; c1ccc(-c2nc(-c3ccccc3)nc(-c3ccc4c(c3)sc3ccc(-c5cccc6c5sc5cccc(-n7c8ccccc8c8ccccc87)c56)cc34)n2)cc1]

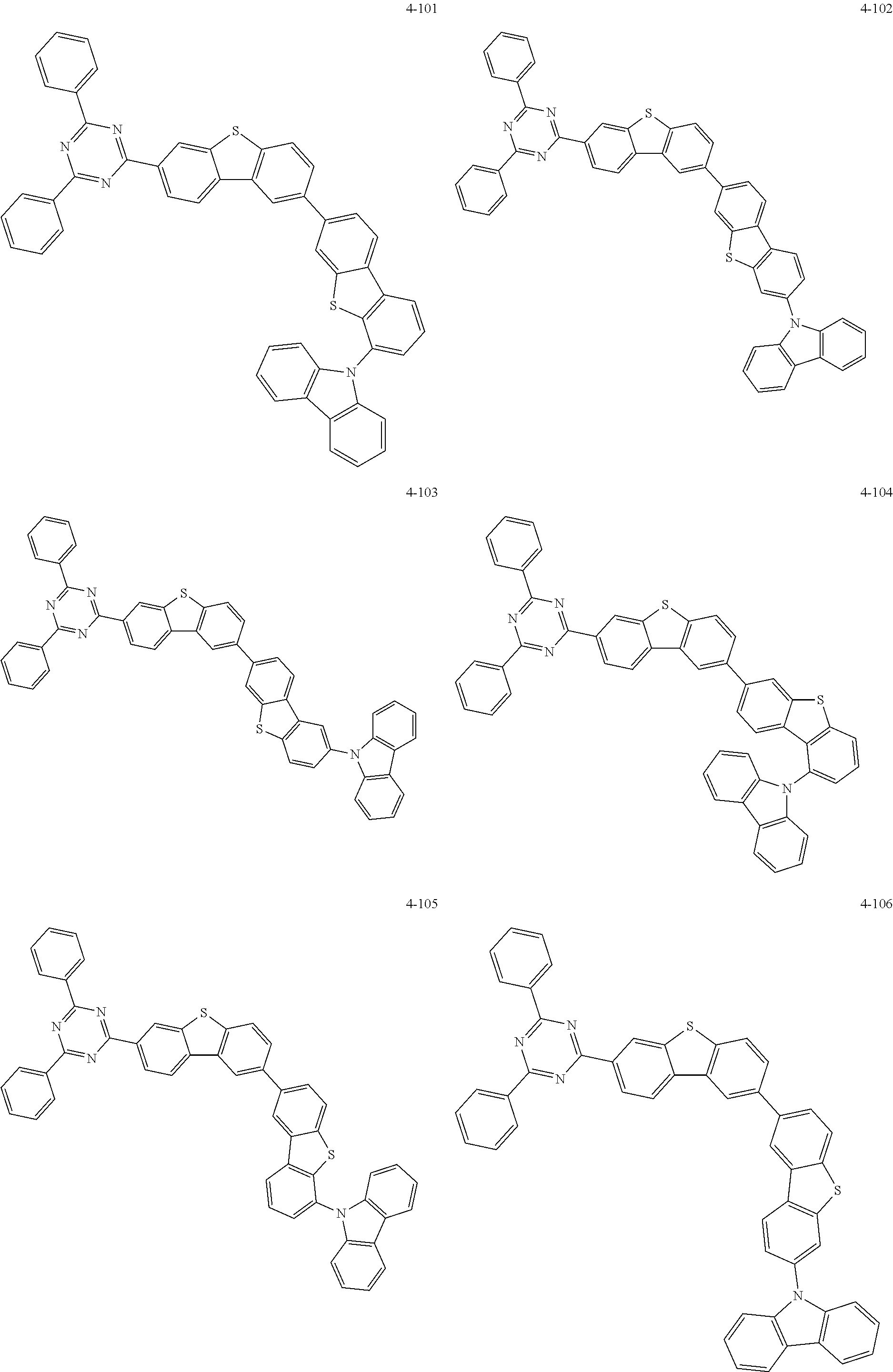

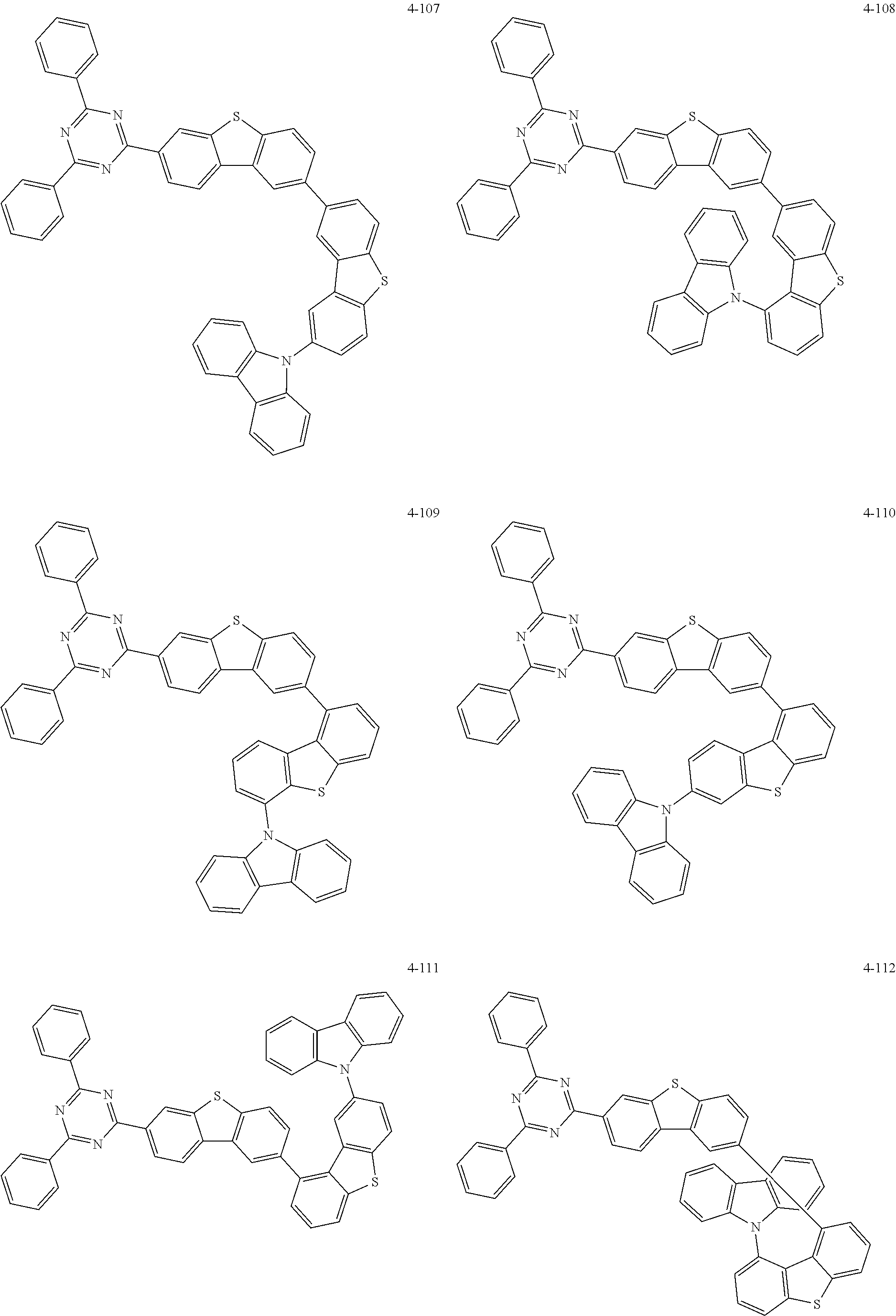

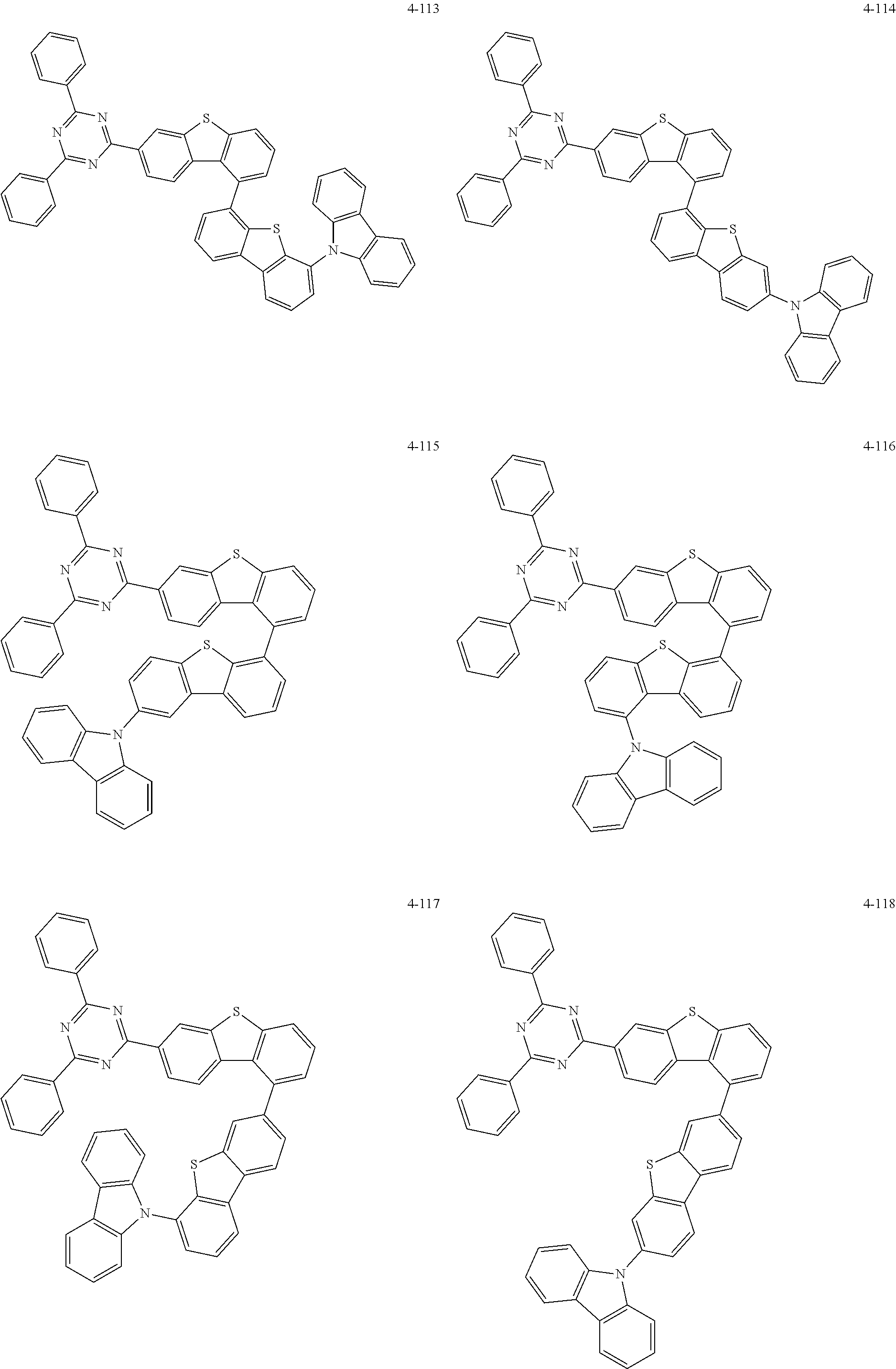

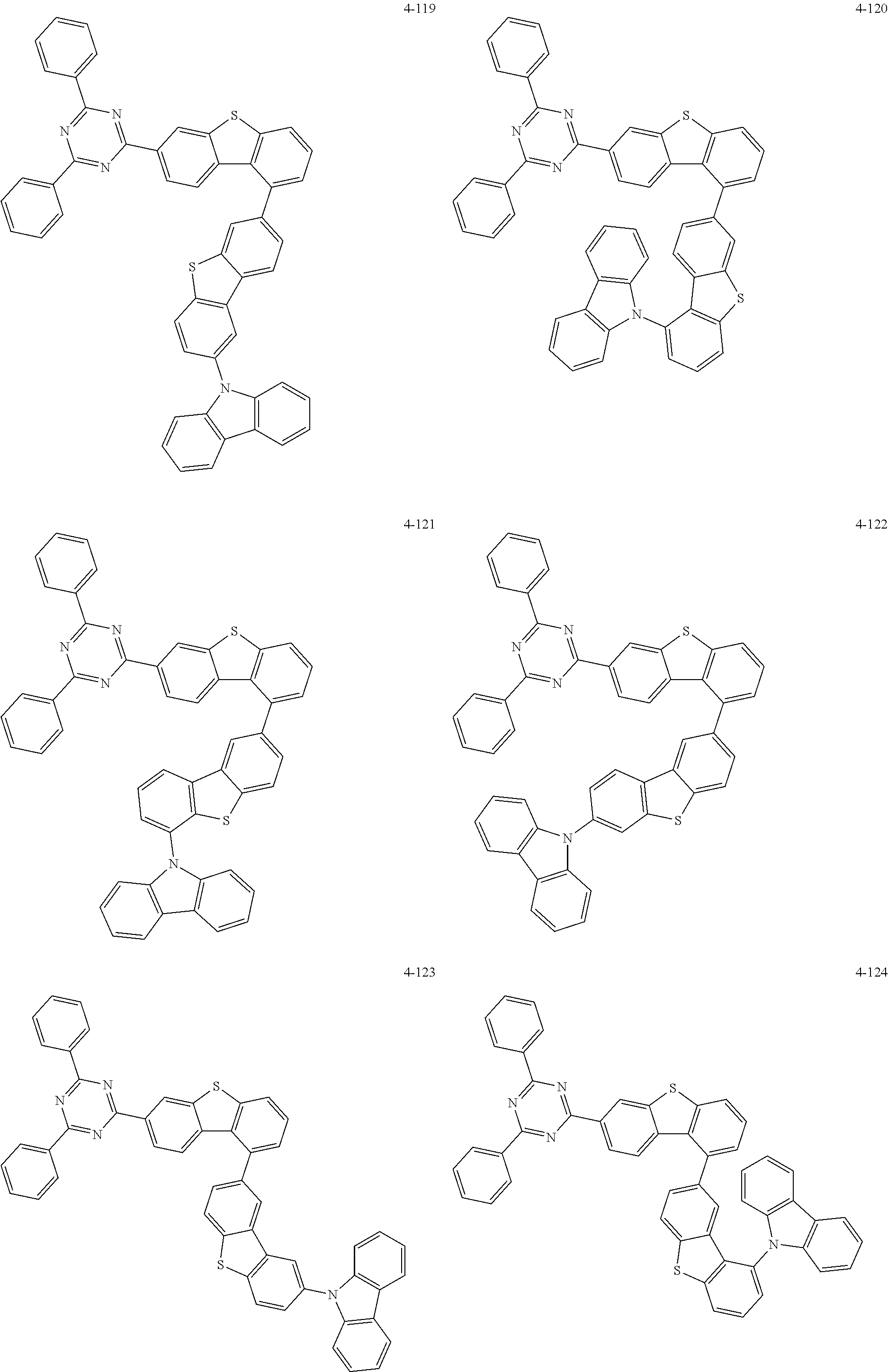

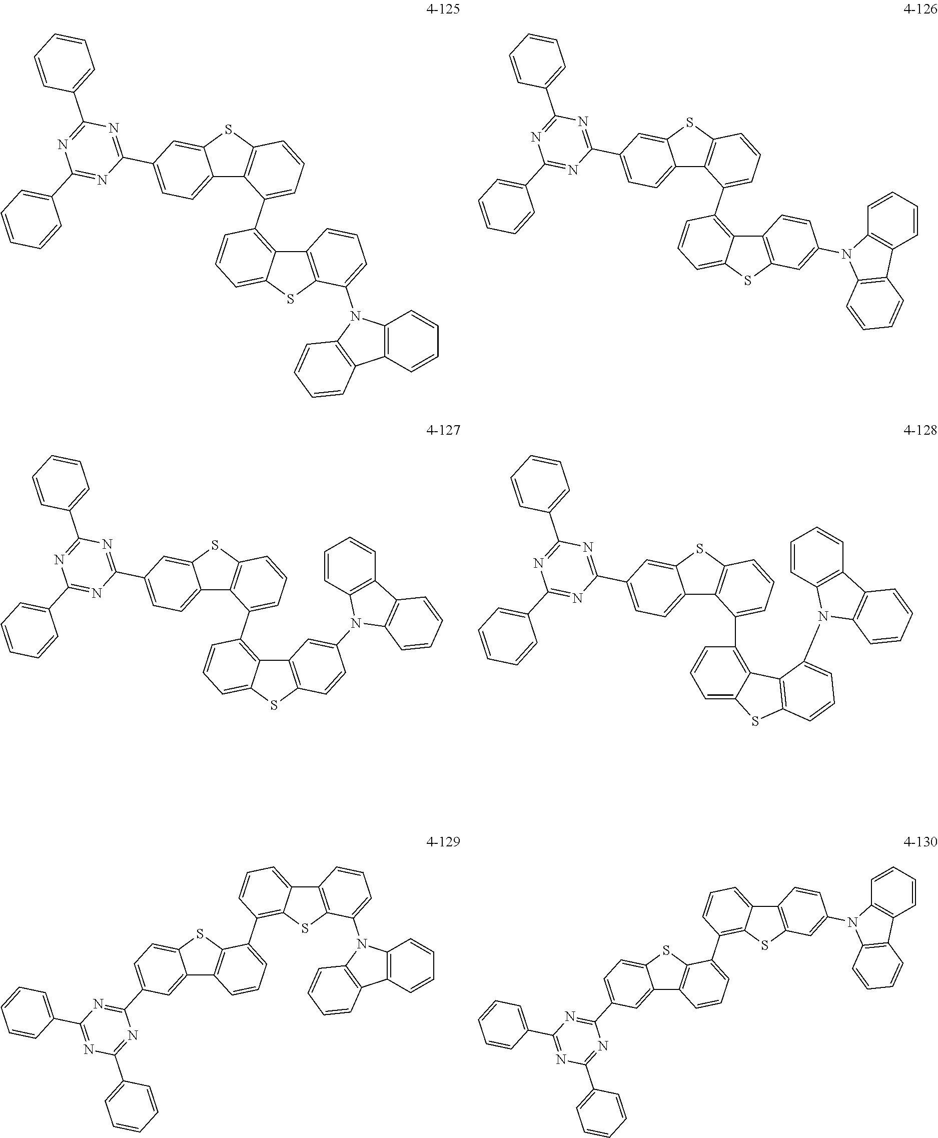

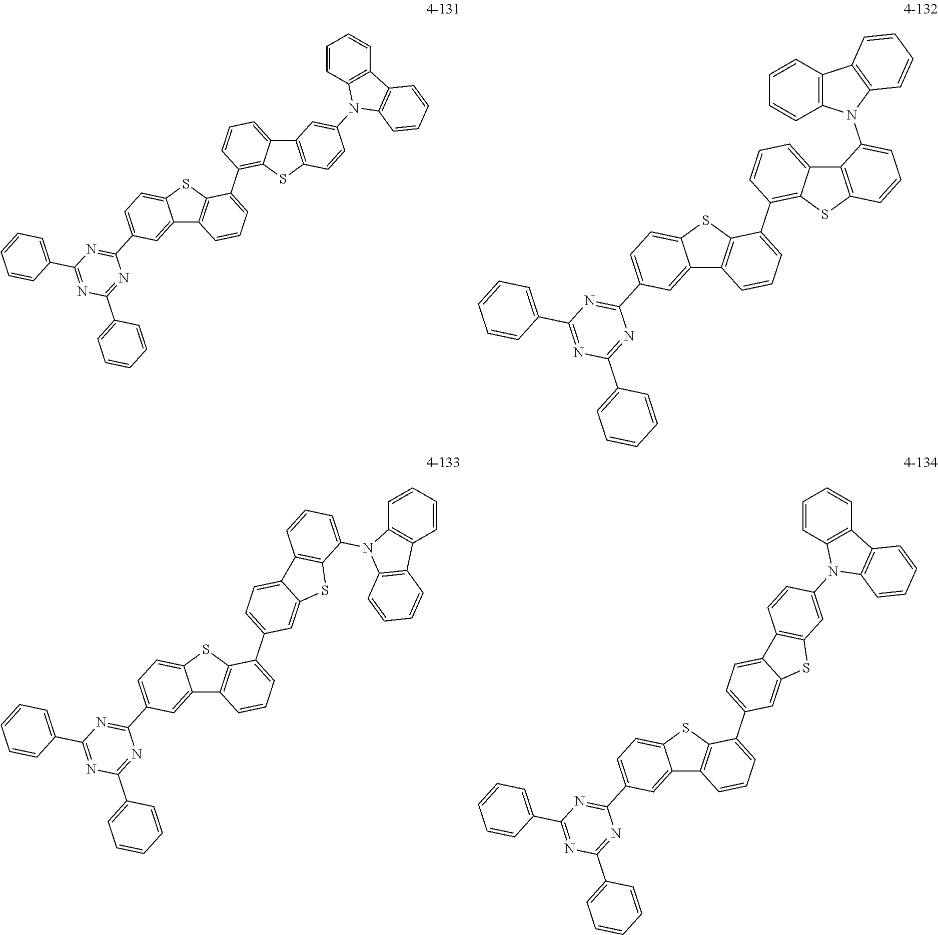 [SMILES: c1ccc(-c2nc(-c3ccccc3)nc(-c3ccc4sc5c(-c6cccc7c6sc6ccc(-n8c9ccccc9c9ccccc98)cc67)cccc5c4c3)n2)cc1 ; c1ccc(-c2nc(-c3ccccc3)nc(-c3ccc4sc5c(-c6cccc7c6sc6cccc(-n8c9ccccc9c9ccccc98)c67)cccc5c4c3)n2)cc1 ; c1ccc(-c2nc(-c3ccccc3)nc(-c3ccc4sc5c(-c6ccc7c(c6)sc6c(-n8c9ccccc9c9ccccc98)cccc67)cccc5c4c3)n2)cc1 ; c1ccc(-c2nc(-c3ccccc3)nc(-c3ccc4sc5c(-c6ccc7c(c6)sc6cc(-n8c9ccccc9c9ccccc98)ccc67)cccc5c4c3)n2)cc1]

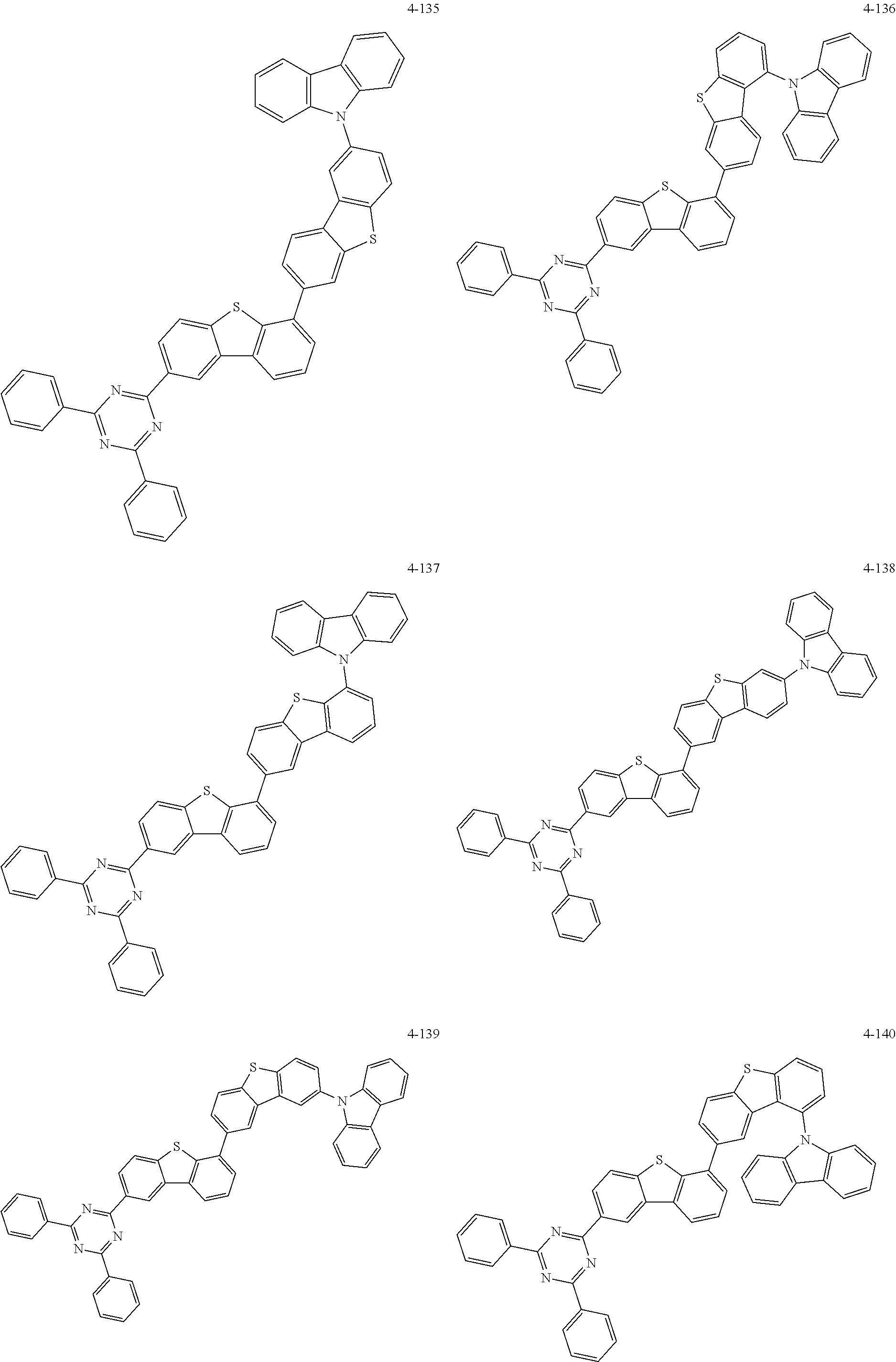

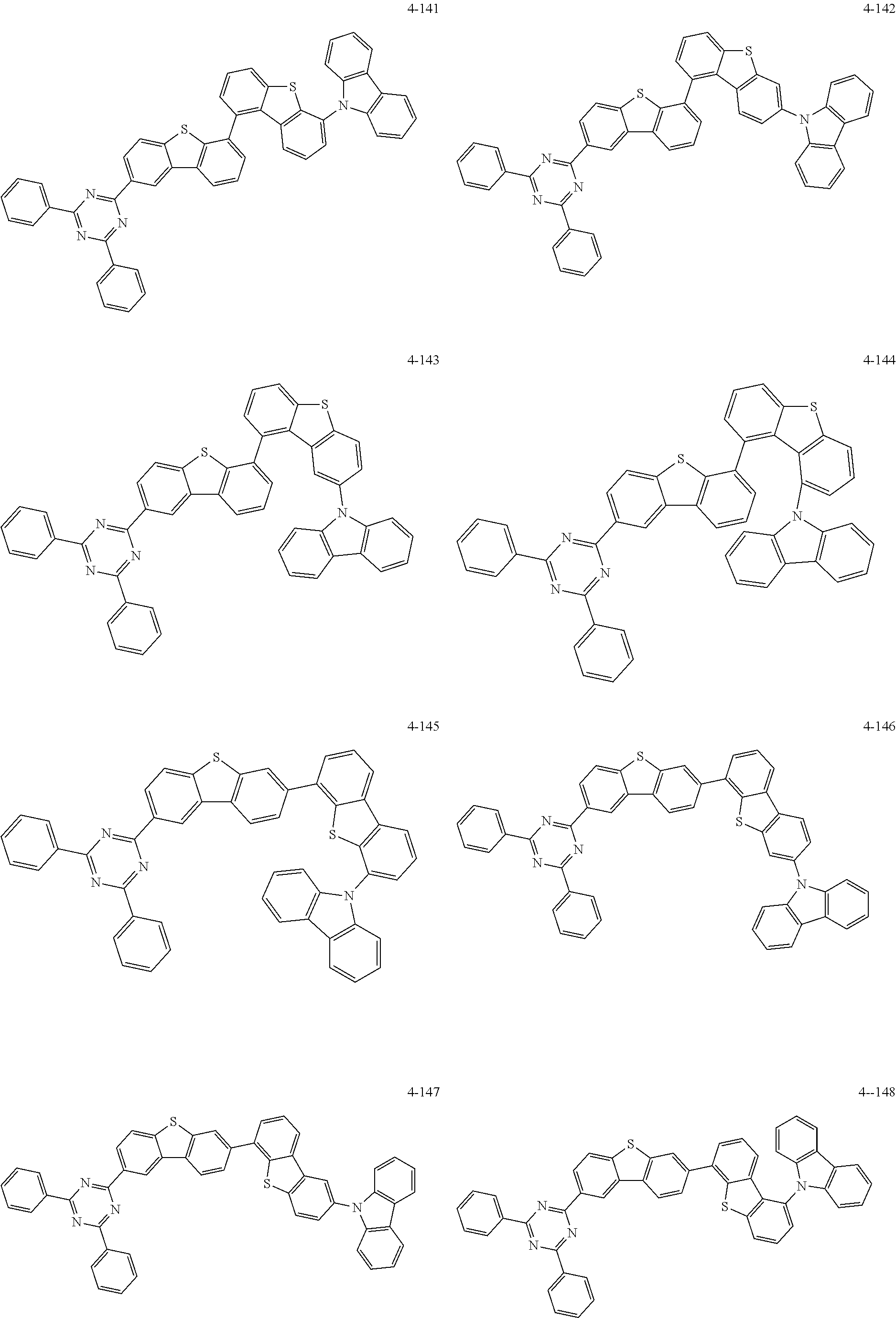

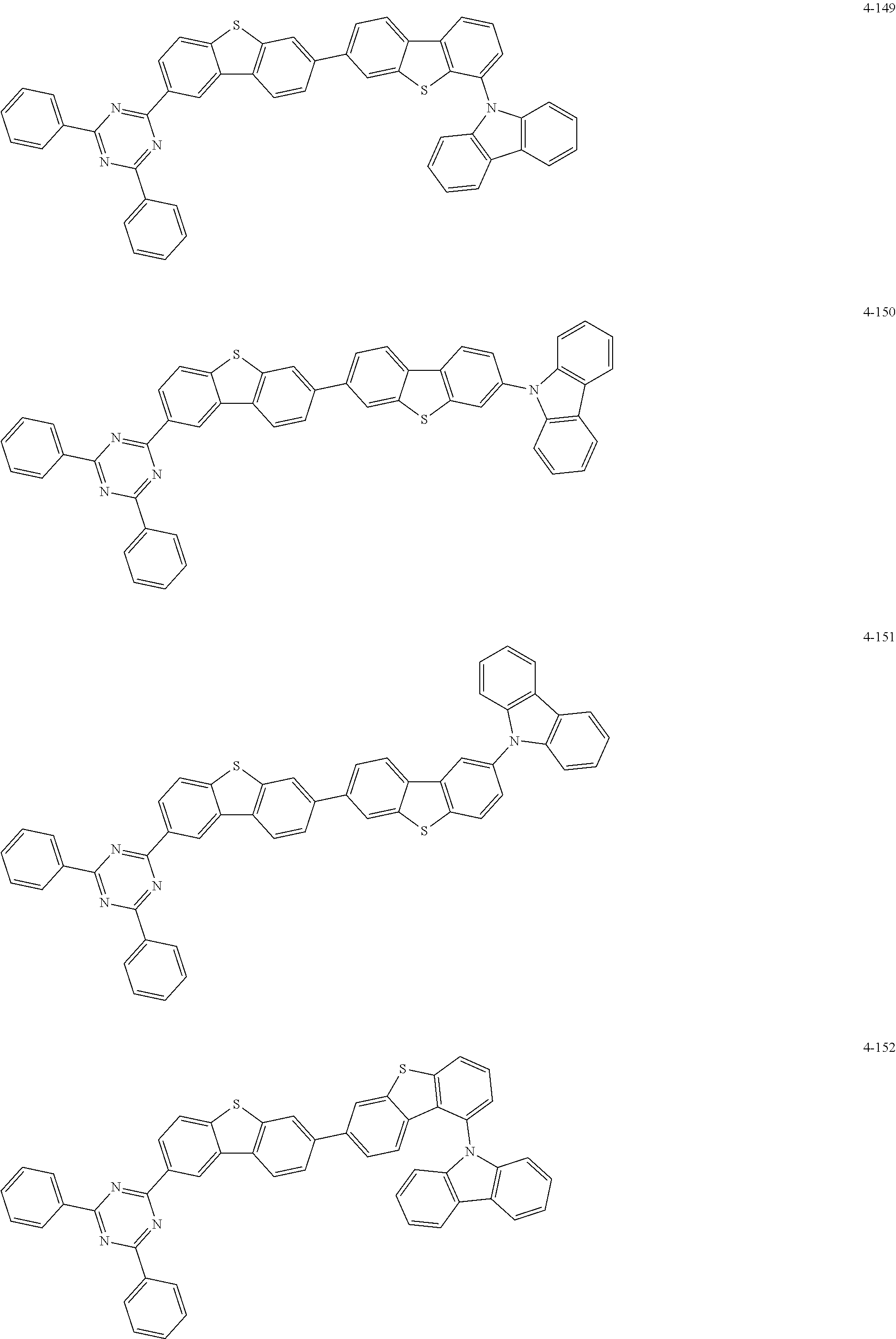 [SMILES: c1ccc(-c2nc(-c3ccccc3)nc(-c3ccc4sc5cc(-c6ccc7c(c6)sc6c(-n8c9ccccc9c9ccccc98)cccc67)ccc5c4c3)n2)cc1 ; c1ccc(-c2nc(-c3ccccc3)nc(-c3ccc4sc5cc(-c6ccc7c(c6)sc6cc(-n8c9ccccc9c9ccccc98)ccc67)ccc5c4c3)n2)cc1 ; c1ccc(-c2nc(-c3ccccc3)nc(-c3ccc4sc5cc(-c6ccc7c(c6)sc6ccc(-n8c9ccccc9c9ccccc98)cc67)ccc5c4c3)n2)cc1 ; c1ccc(-c2nc(-c3ccccc3)nc(-c3ccc4sc5cc(-c6ccc7c(c6)sc6cccc(-n8c9ccccc9c9ccccc98)c67)ccc5c4c3)n2)cc1]

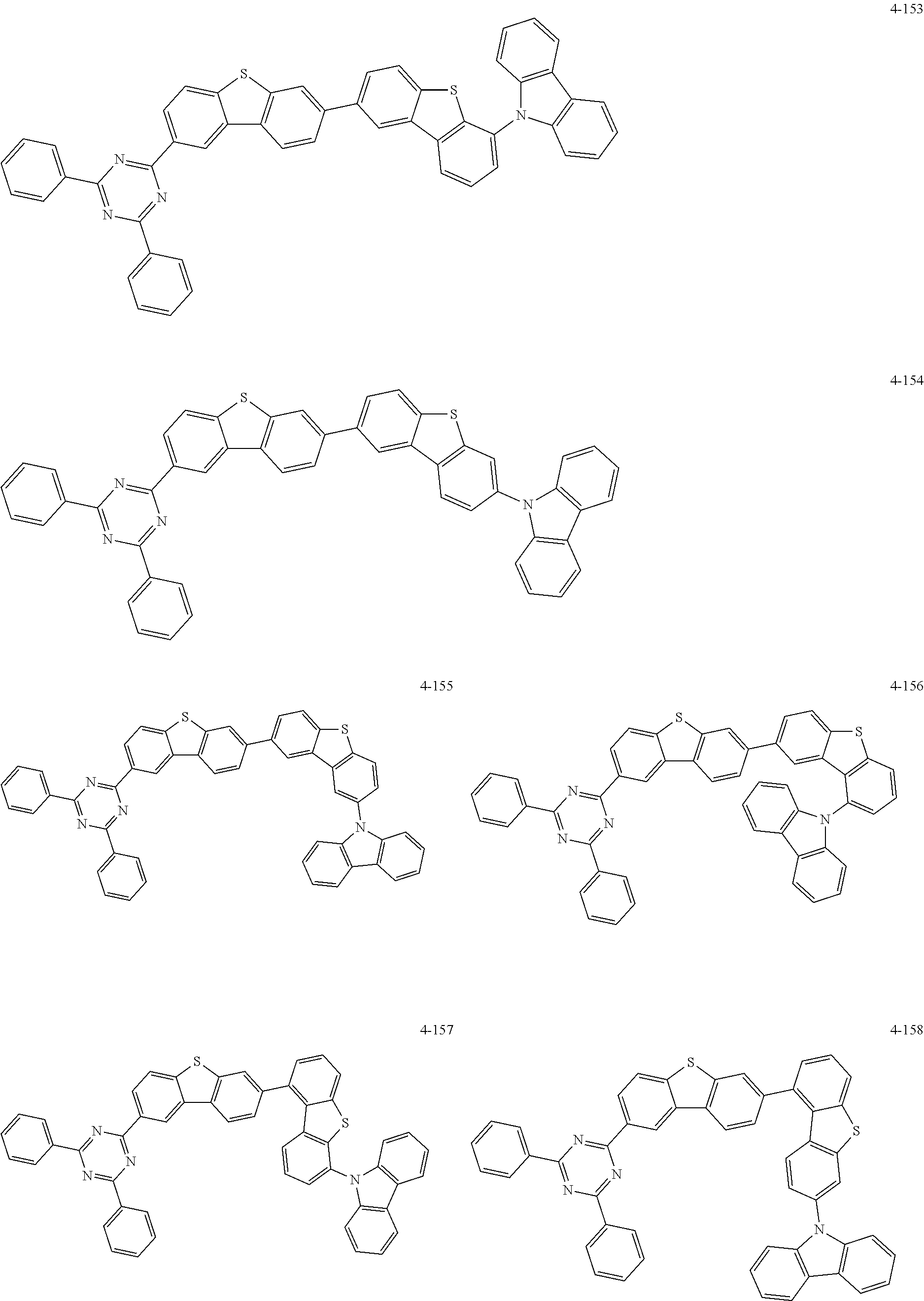

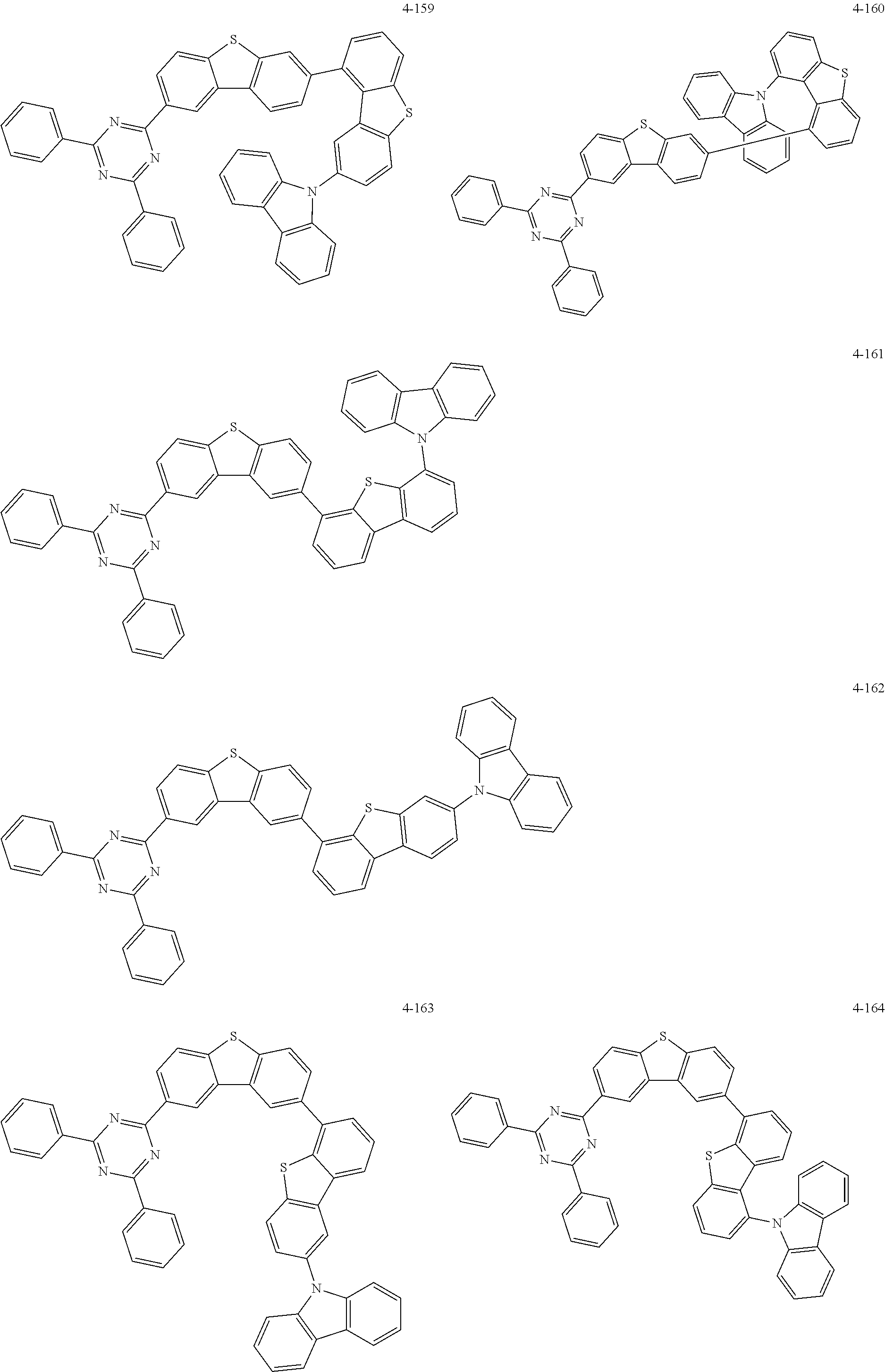

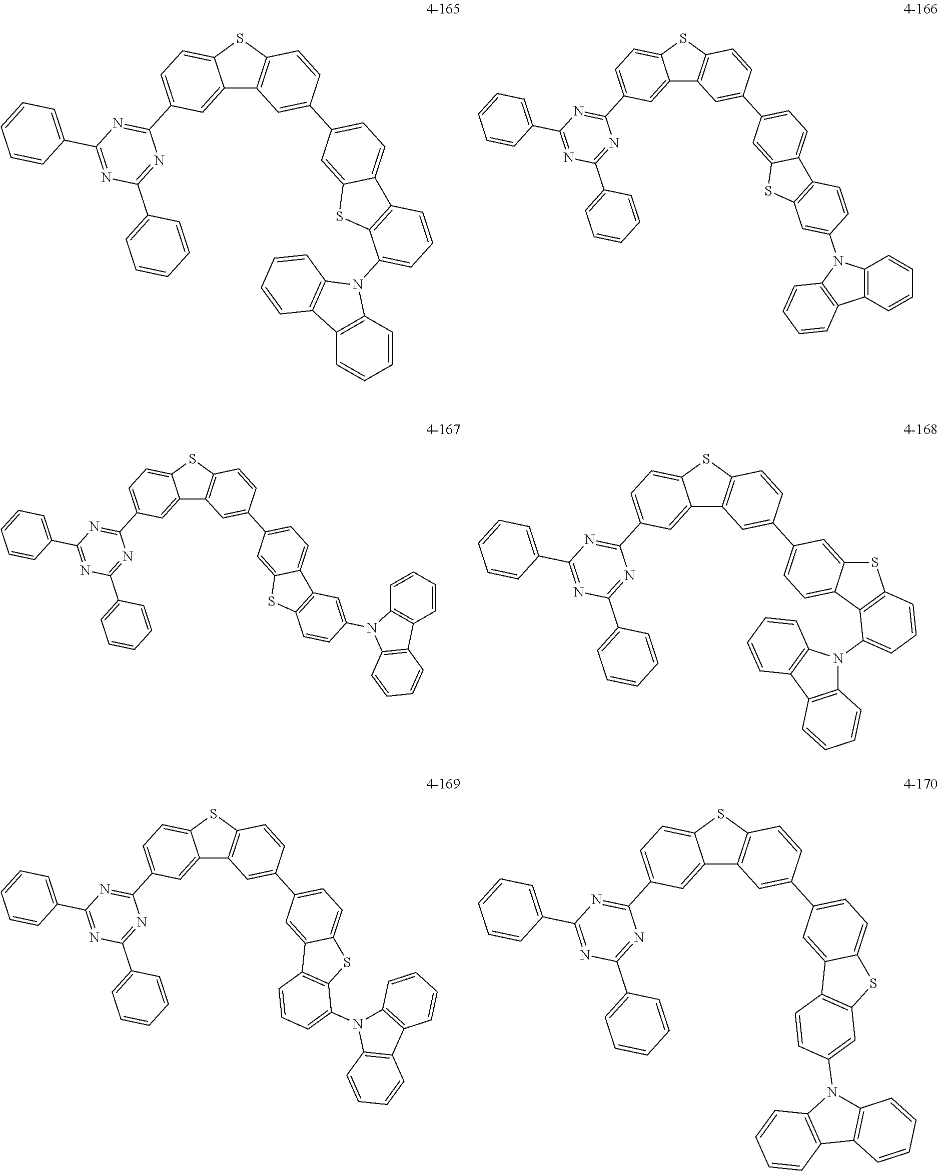

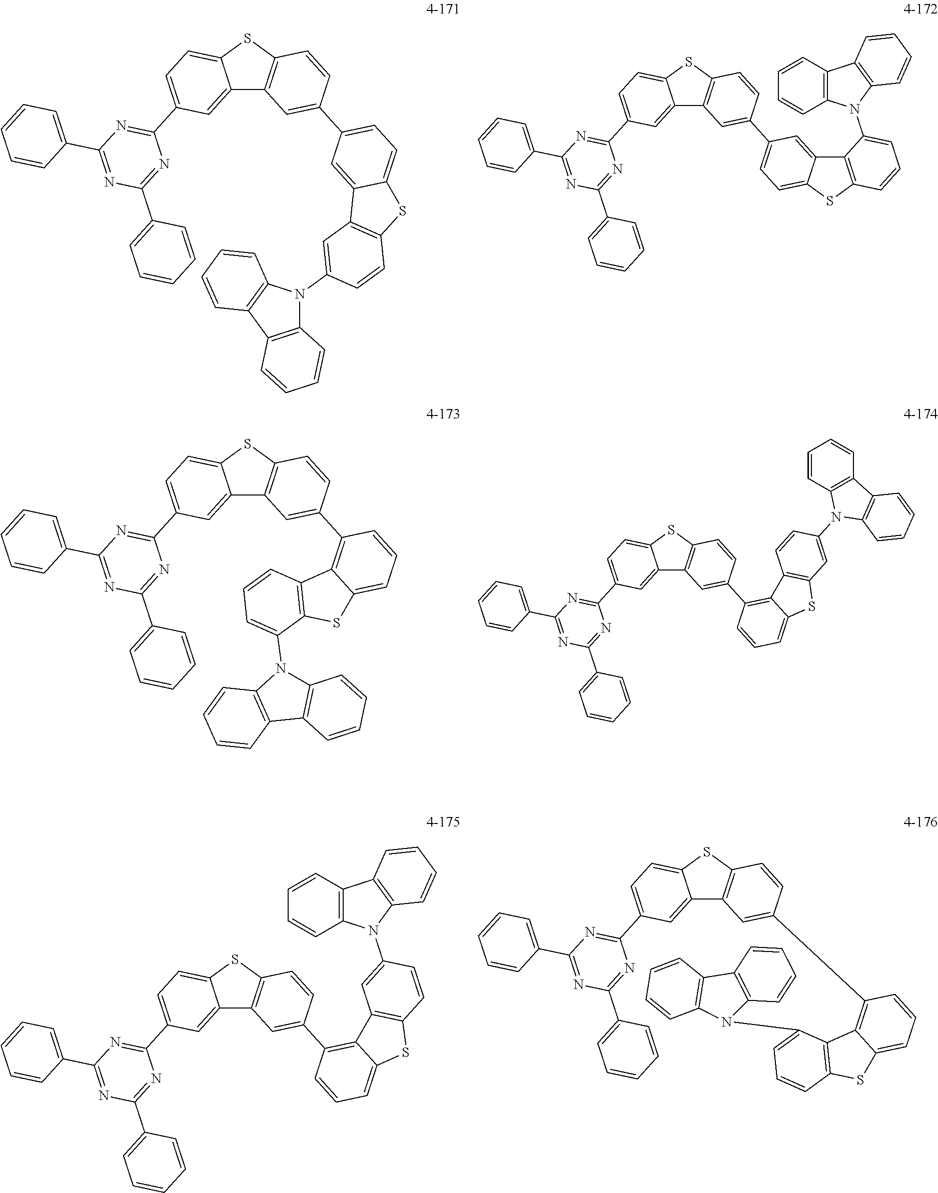

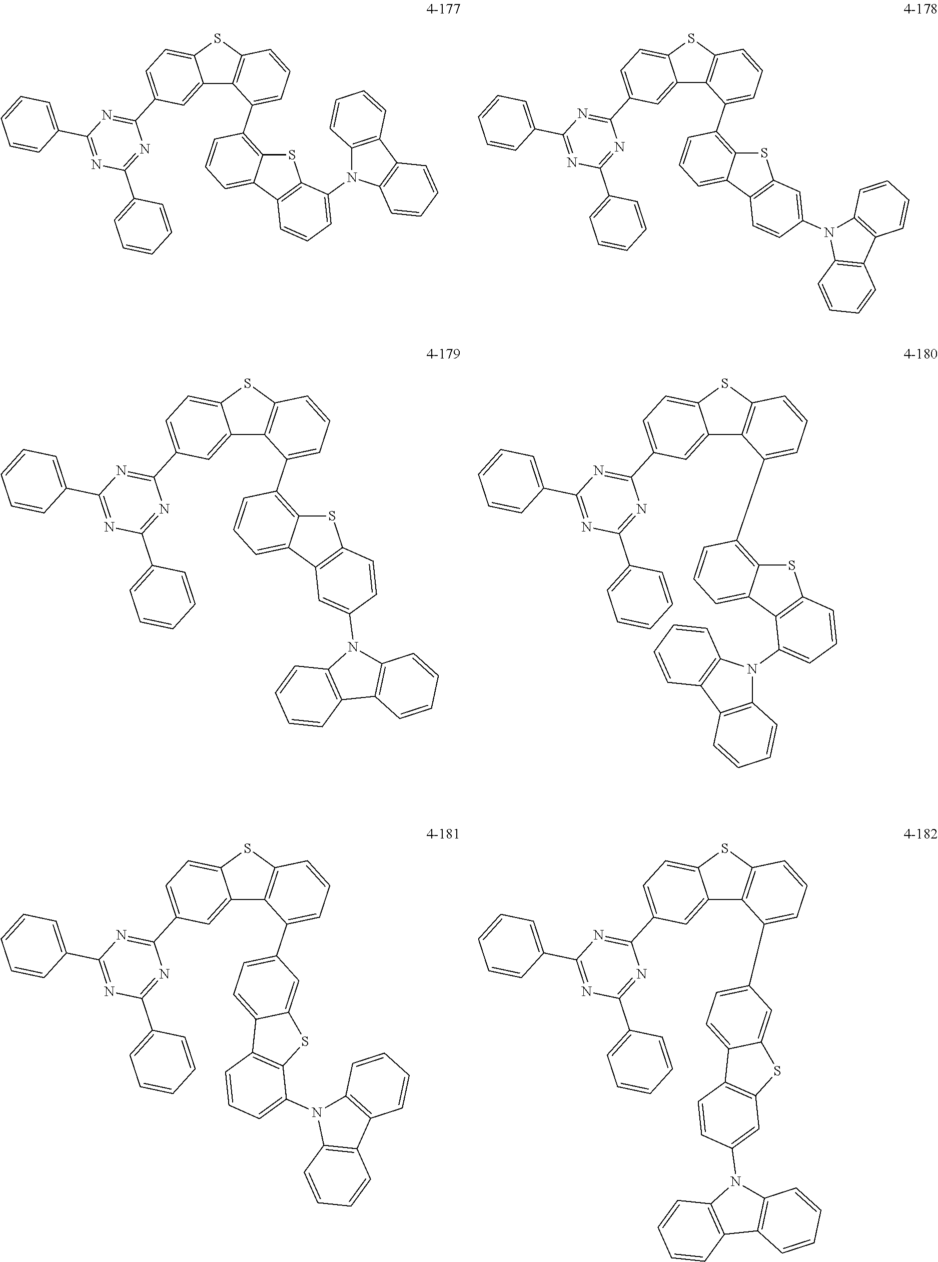

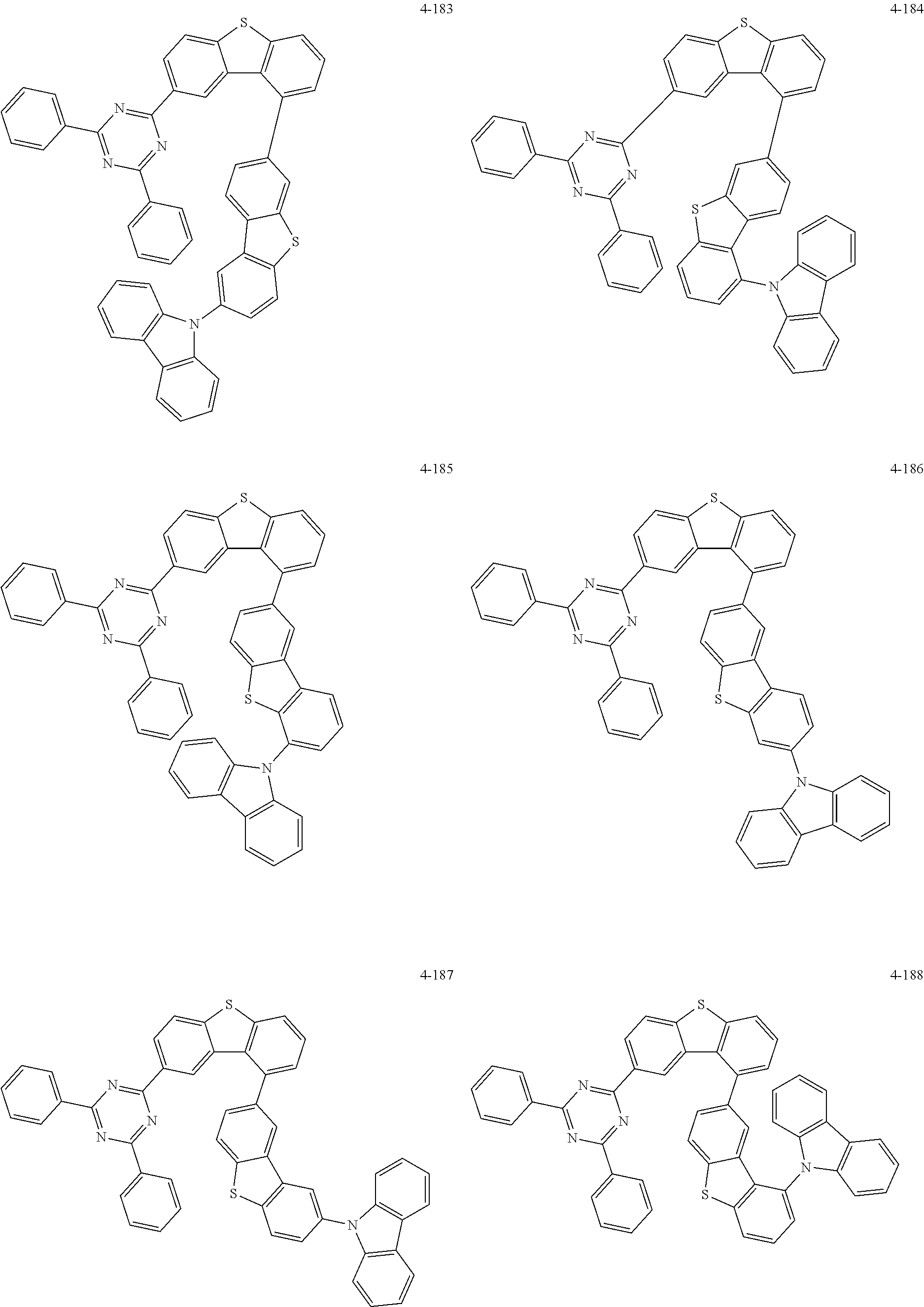

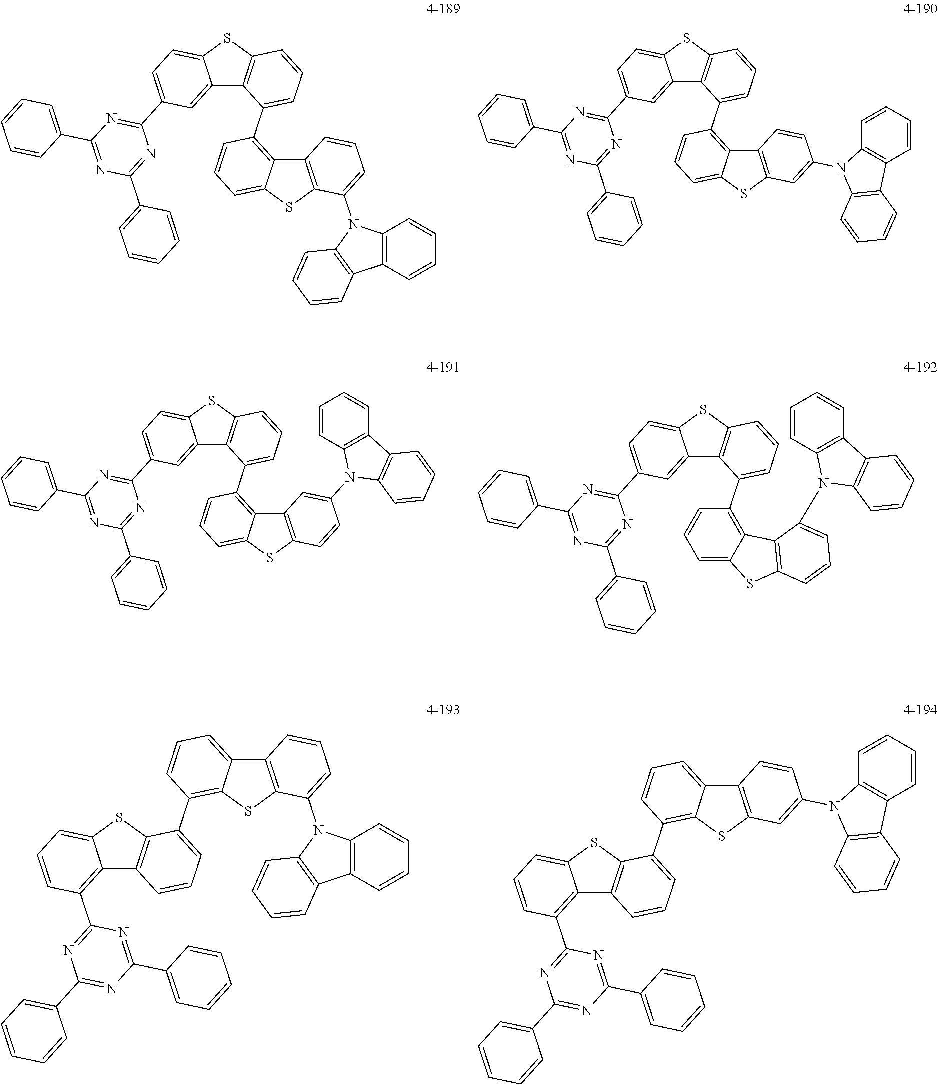

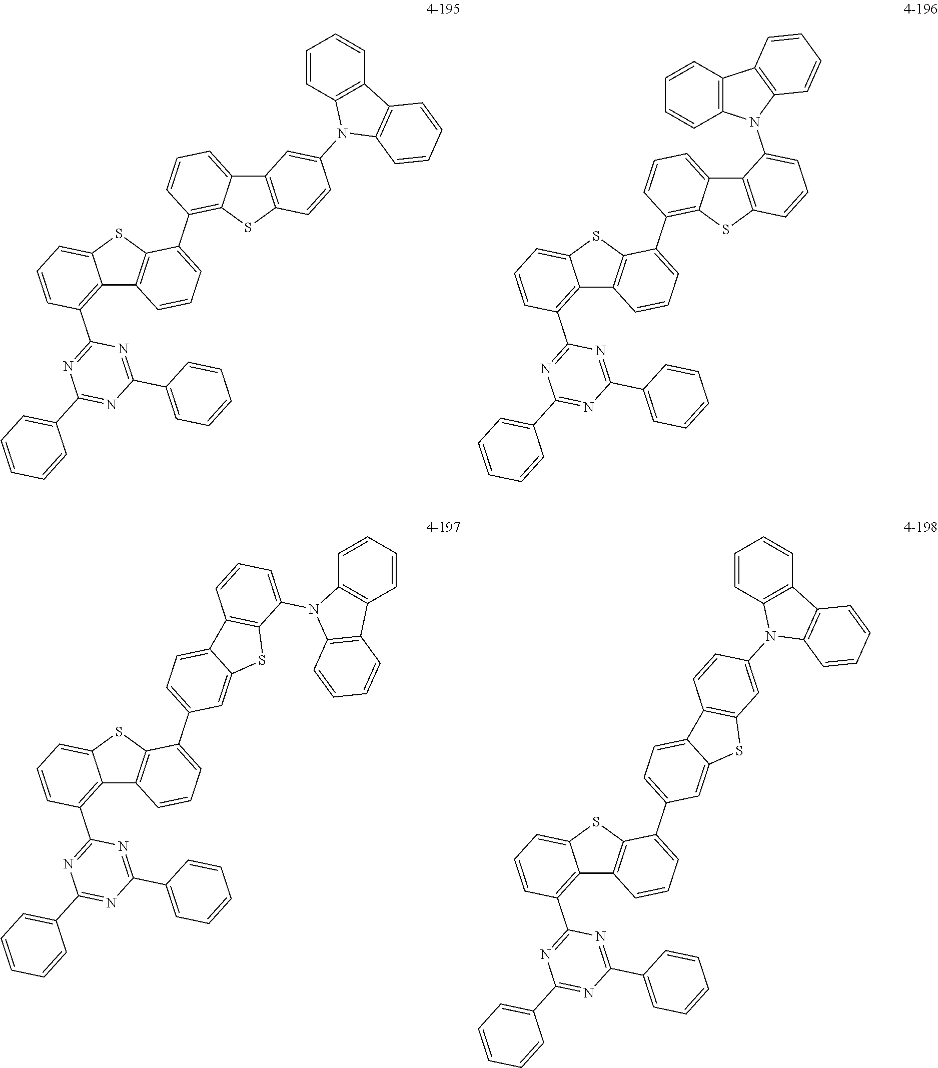 [SMILES: c1ccc(-c2nc(-c3ccccc3)nc(-c3cccc4sc5c(-c6cccc7c6sc6ccc(-n8c9ccccc9c9ccccc98)cc67)cccc5c34)n2)cc1 ; c1ccc(-c2nc(-c3ccccc3)nc(-c3cccc4sc5c(-c6cccc7c6sc6cccc(-n8c9ccccc9c9ccccc98)c67)cccc5c34)n2)cc1 ; c1ccc(-c2nc(-c3ccccc3)nc(-c3cccc4sc5c(-c6ccc7c(c6)sc6c(-n8c9ccccc9c9ccccc98)cccc67)cccc5c34)n2)cc1 ; c1ccc(-c2nc(-c3ccccc3)nc(-c3cccc4sc5c(-c6ccc7c(c6)sc6cc(-n8c9ccccc9c9ccccc98)ccc67)cccc5c34)n2)cc1]

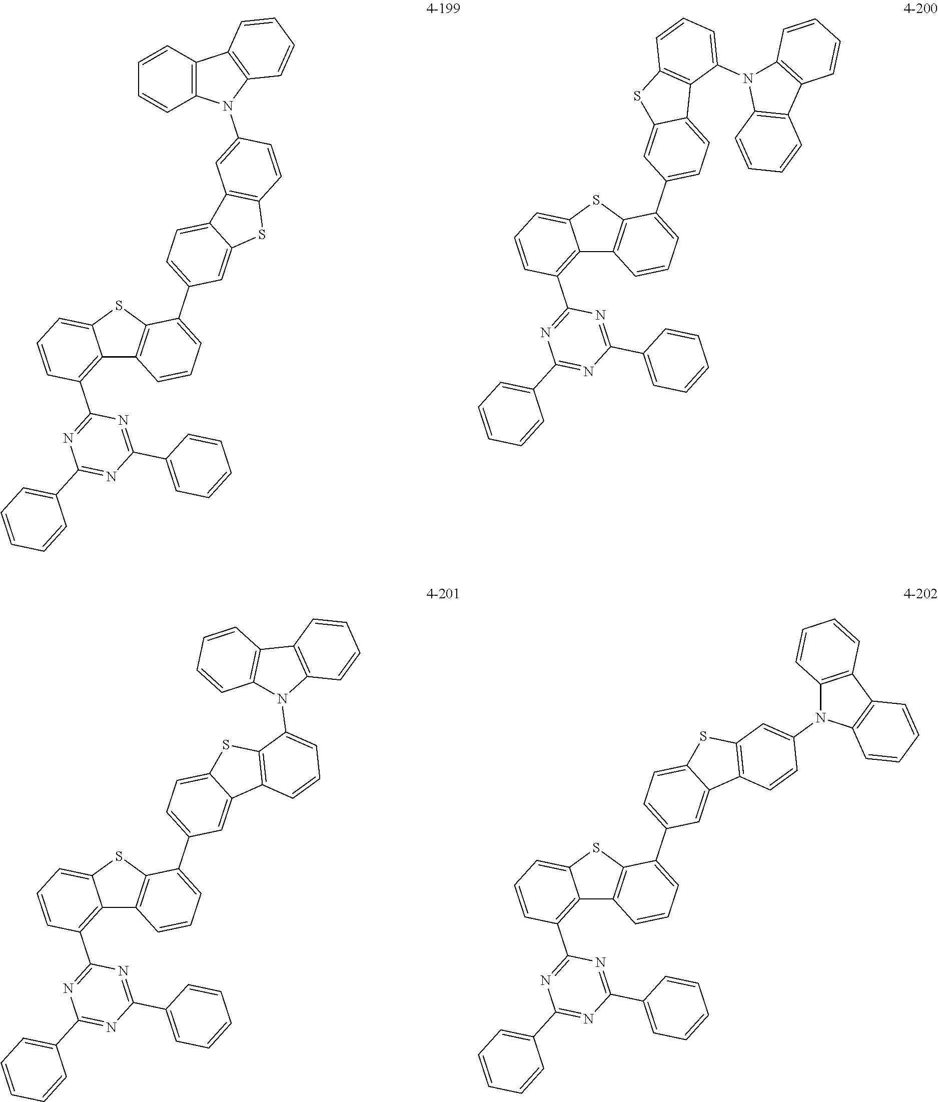 [SMILES: c1ccc(-c2nc(-c3ccccc3)nc(-c3cccc4sc5c(-c6ccc7c(c6)sc6ccc(-n8c9ccccc9c9ccccc98)cc67)cccc5c34)n2)cc1 ; c1ccc(-c2nc(-c3ccccc3)nc(-c3cccc4sc5c(-c6ccc7c(c6)sc6cccc(-n8c9ccccc9c9ccccc98)c67)cccc5c34)n2)cc1 ; c1ccc(-c2nc(-c3ccccc3)nc(-c3cccc4sc5c(-c6ccc7sc8c(-n9c%10ccccc%10c%10ccccc%109)cccc8c7c6)cccc5c34)n2)cc1 ; c1ccc(-c2nc(-c3ccccc3)nc(-c3cccc4sc5c(-c6ccc7sc8cc(-n9c%10ccccc%10c%10ccccc%109)ccc8c7c6)cccc5c34)n2)cc1]

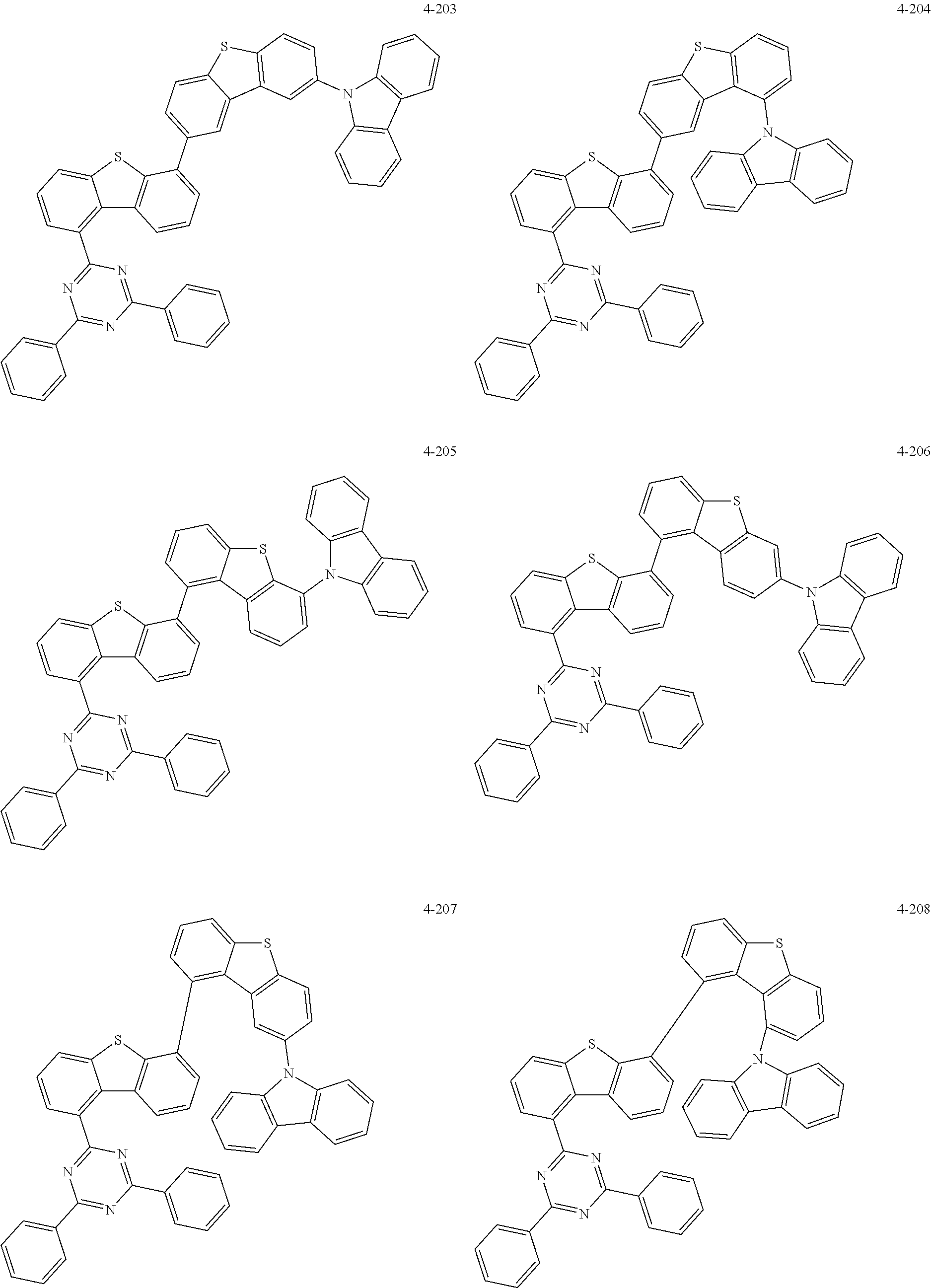

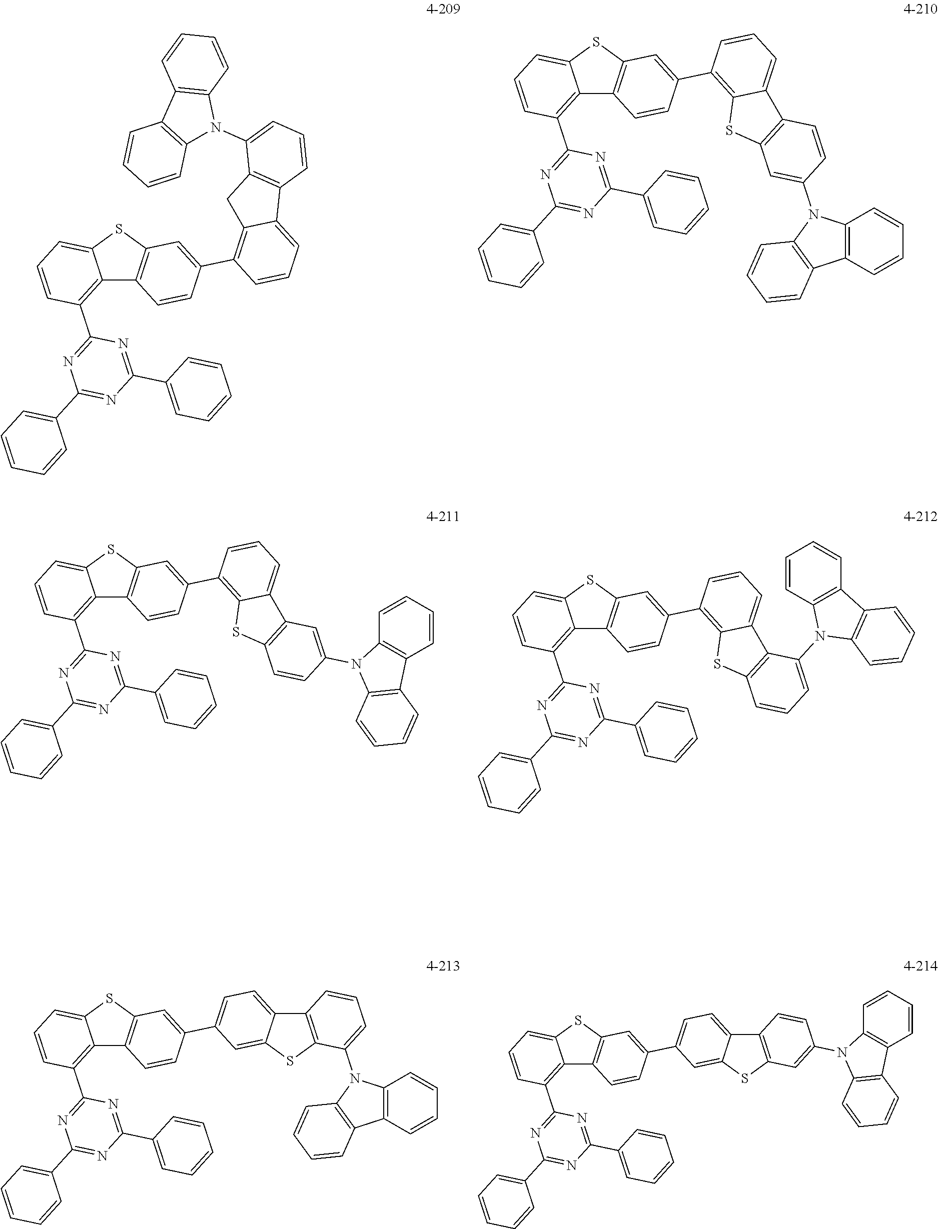

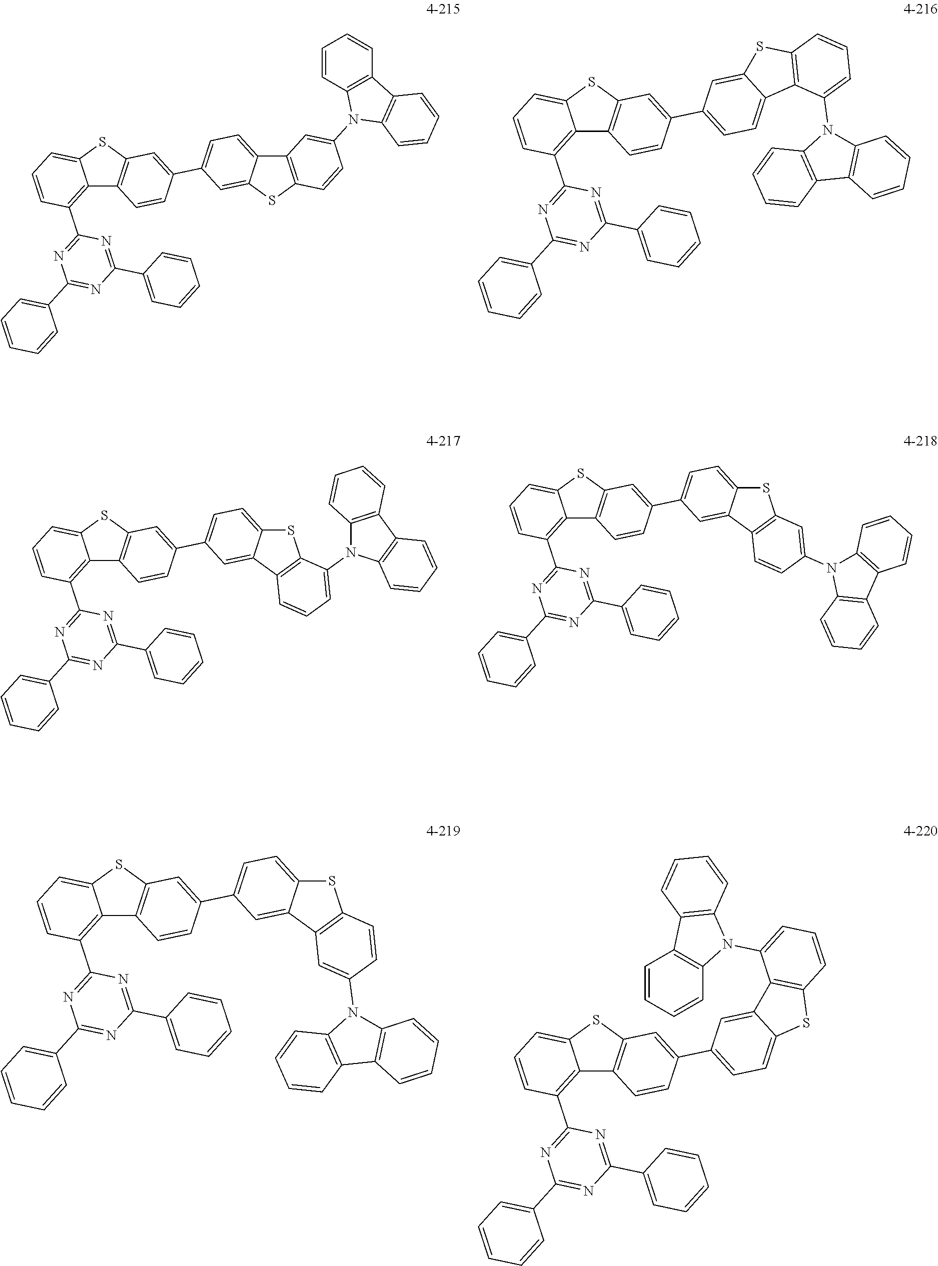

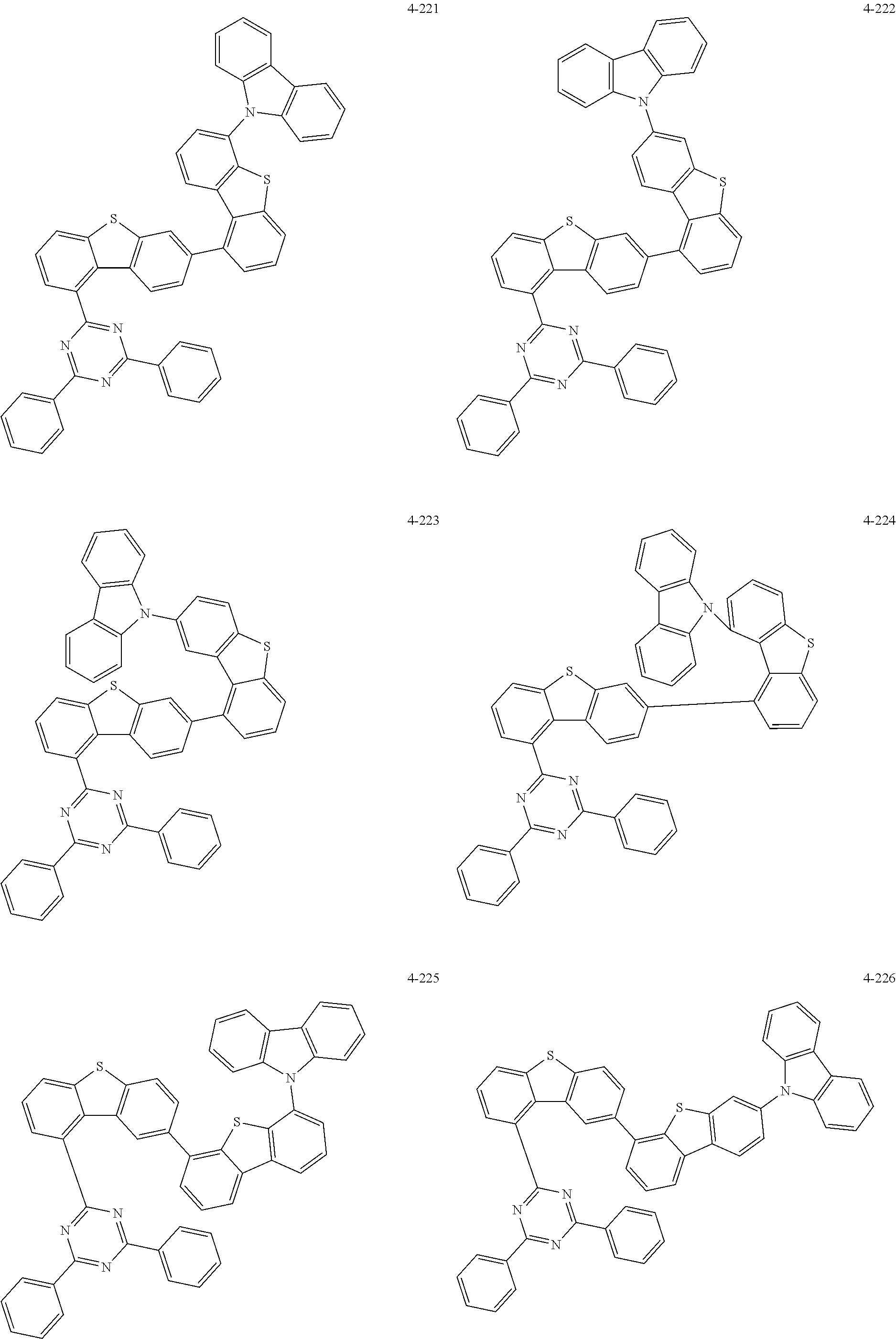

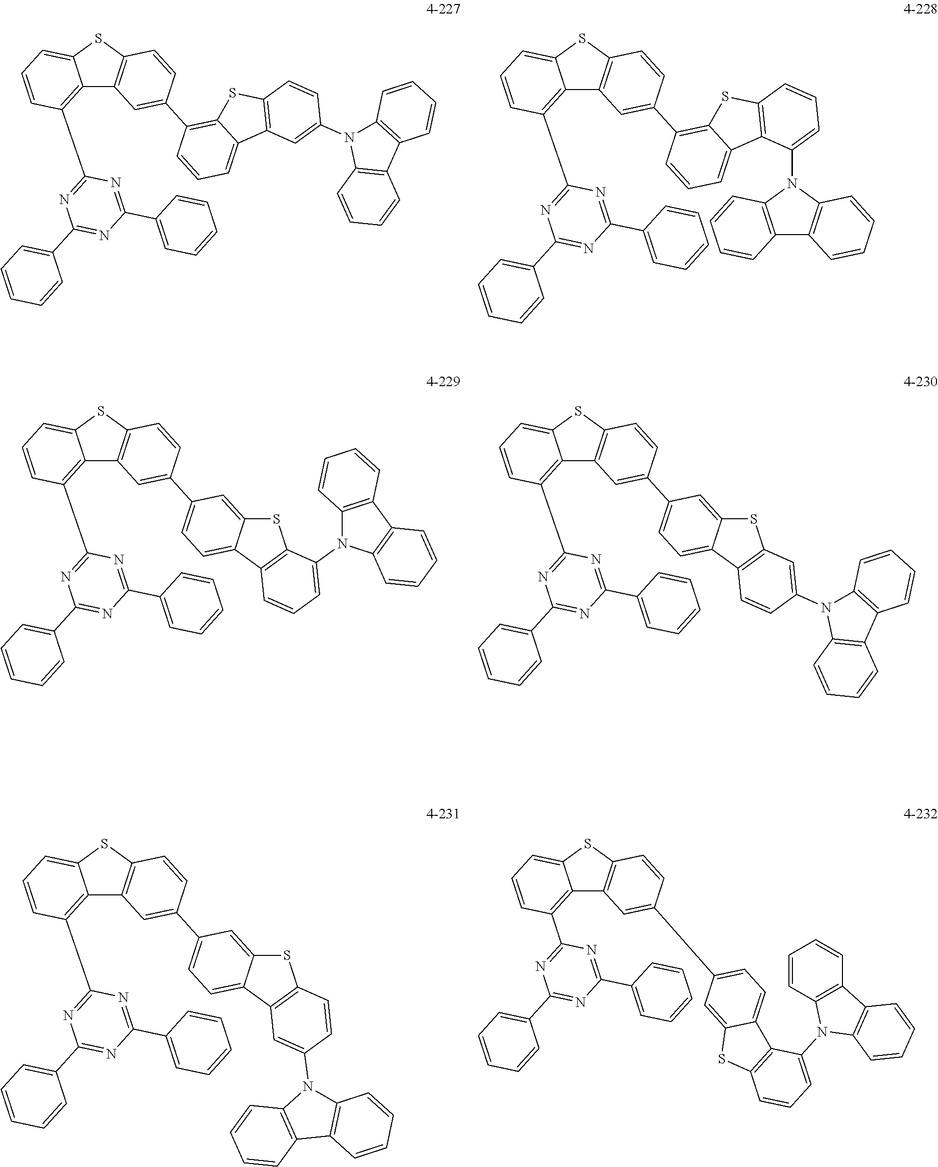

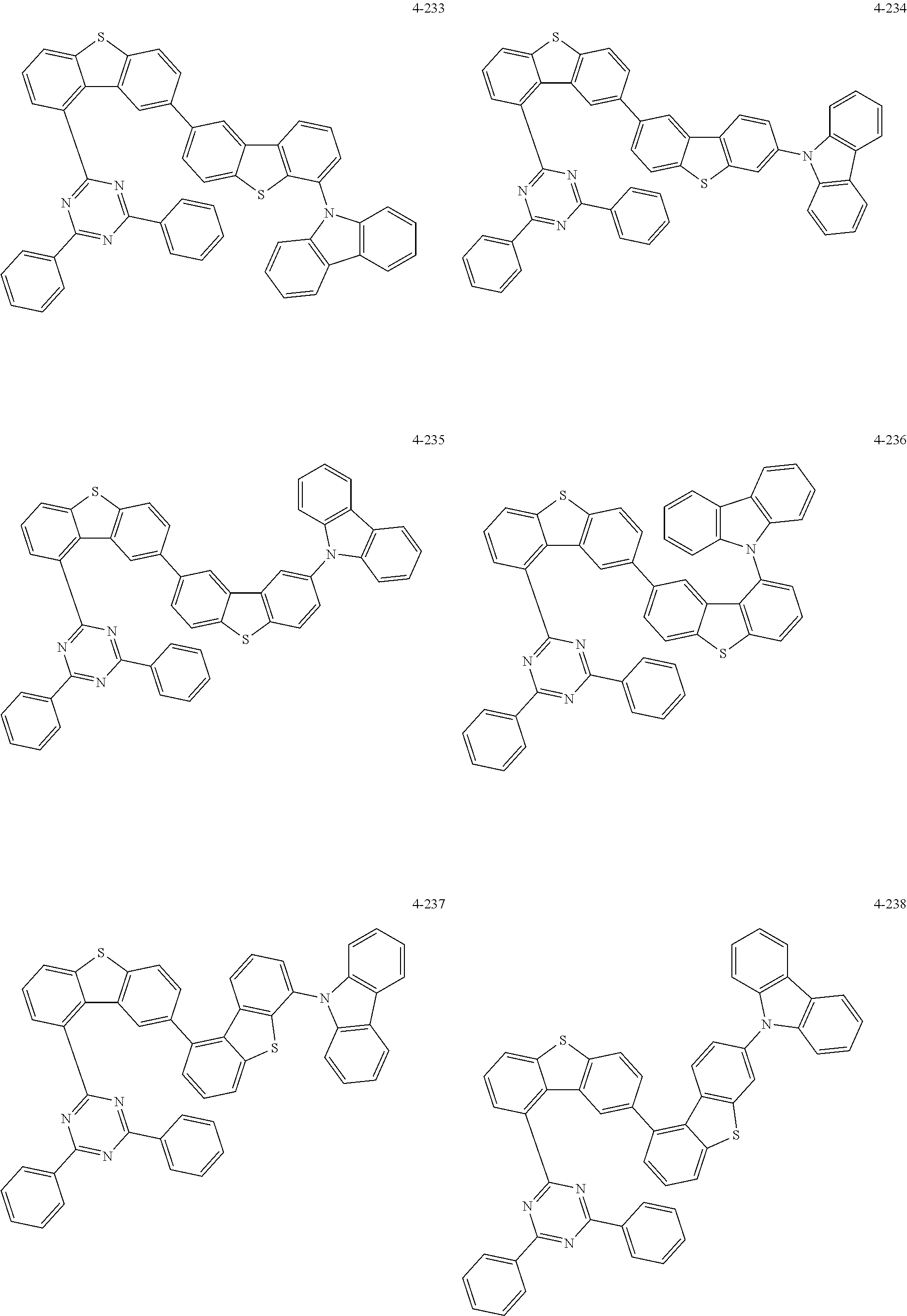

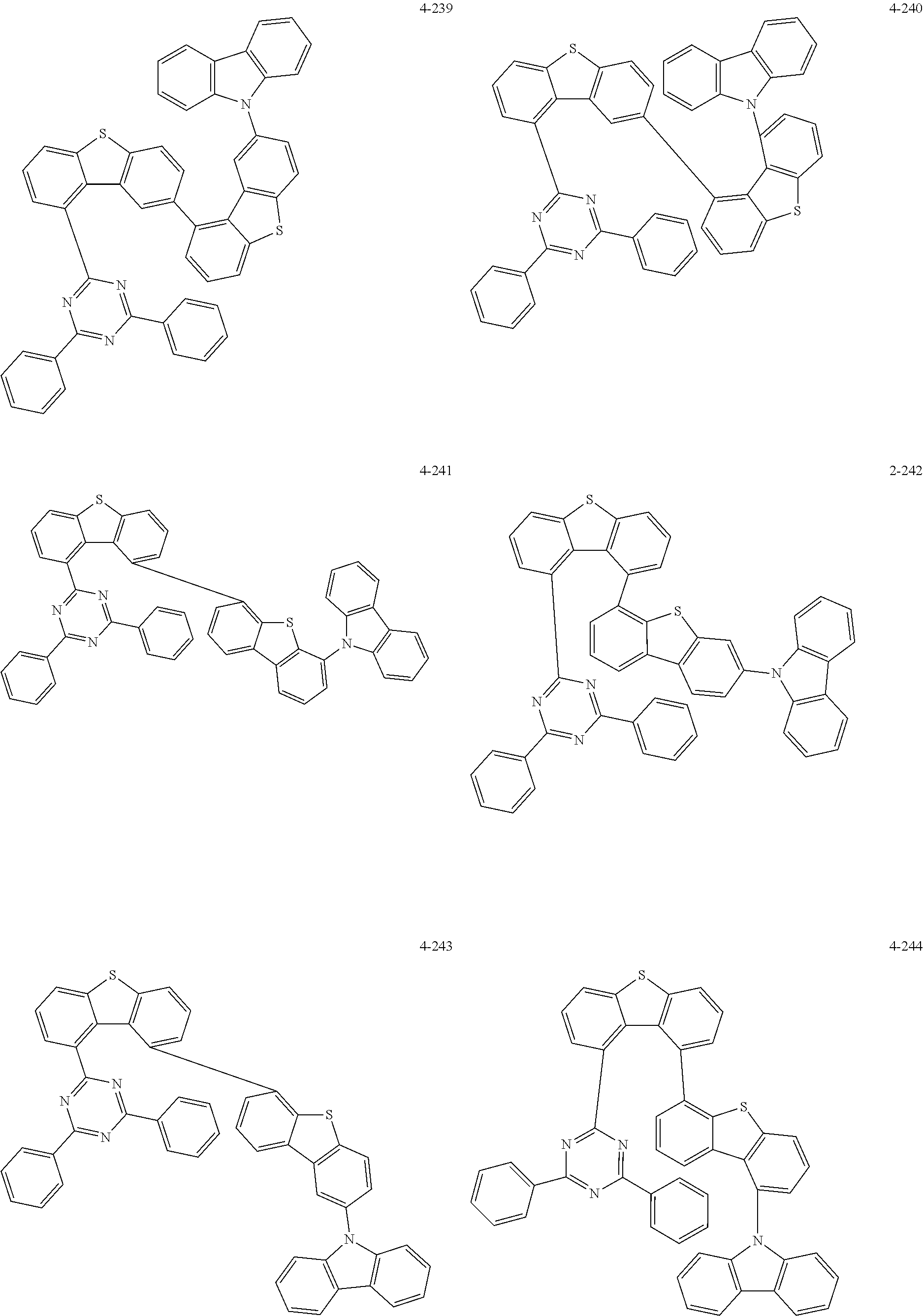

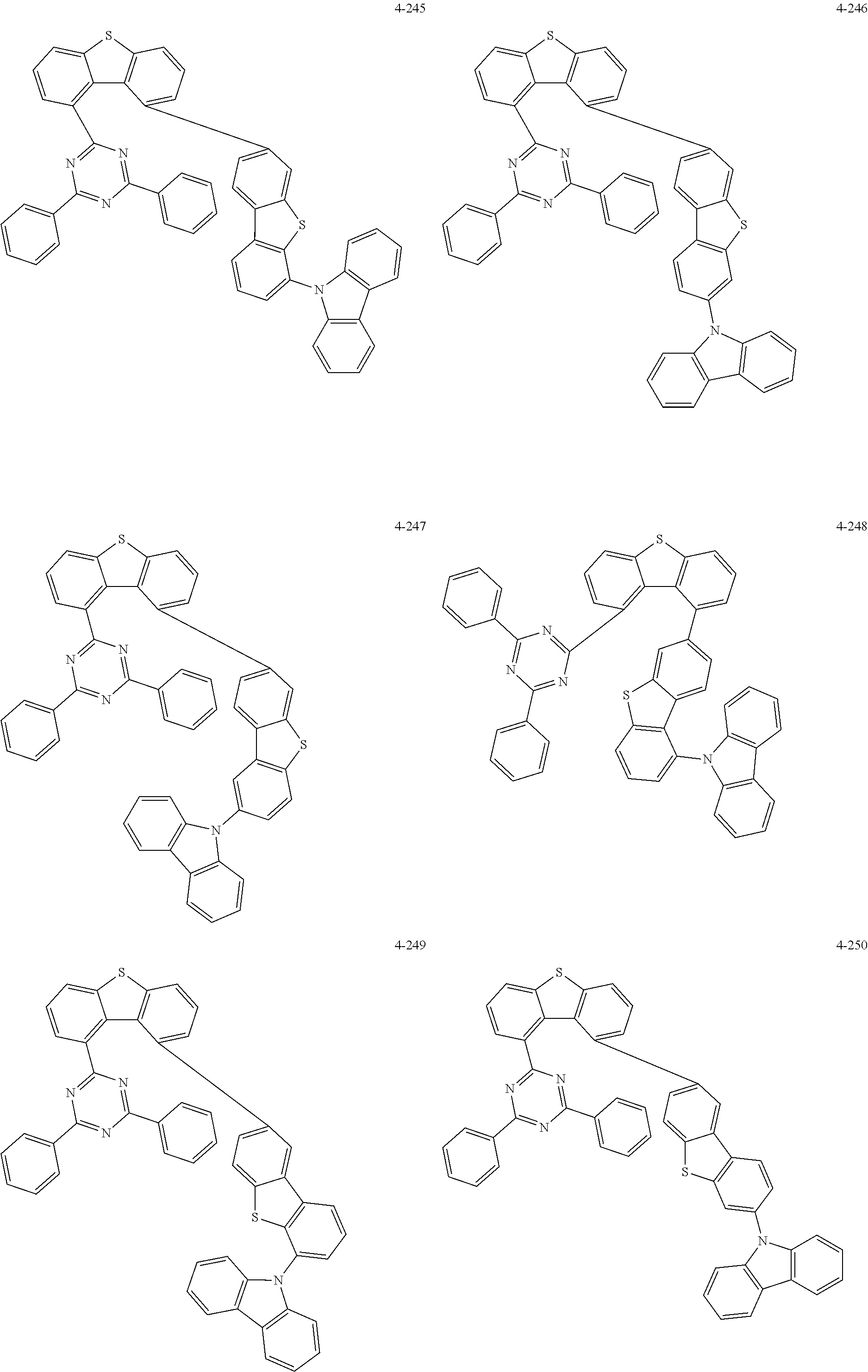

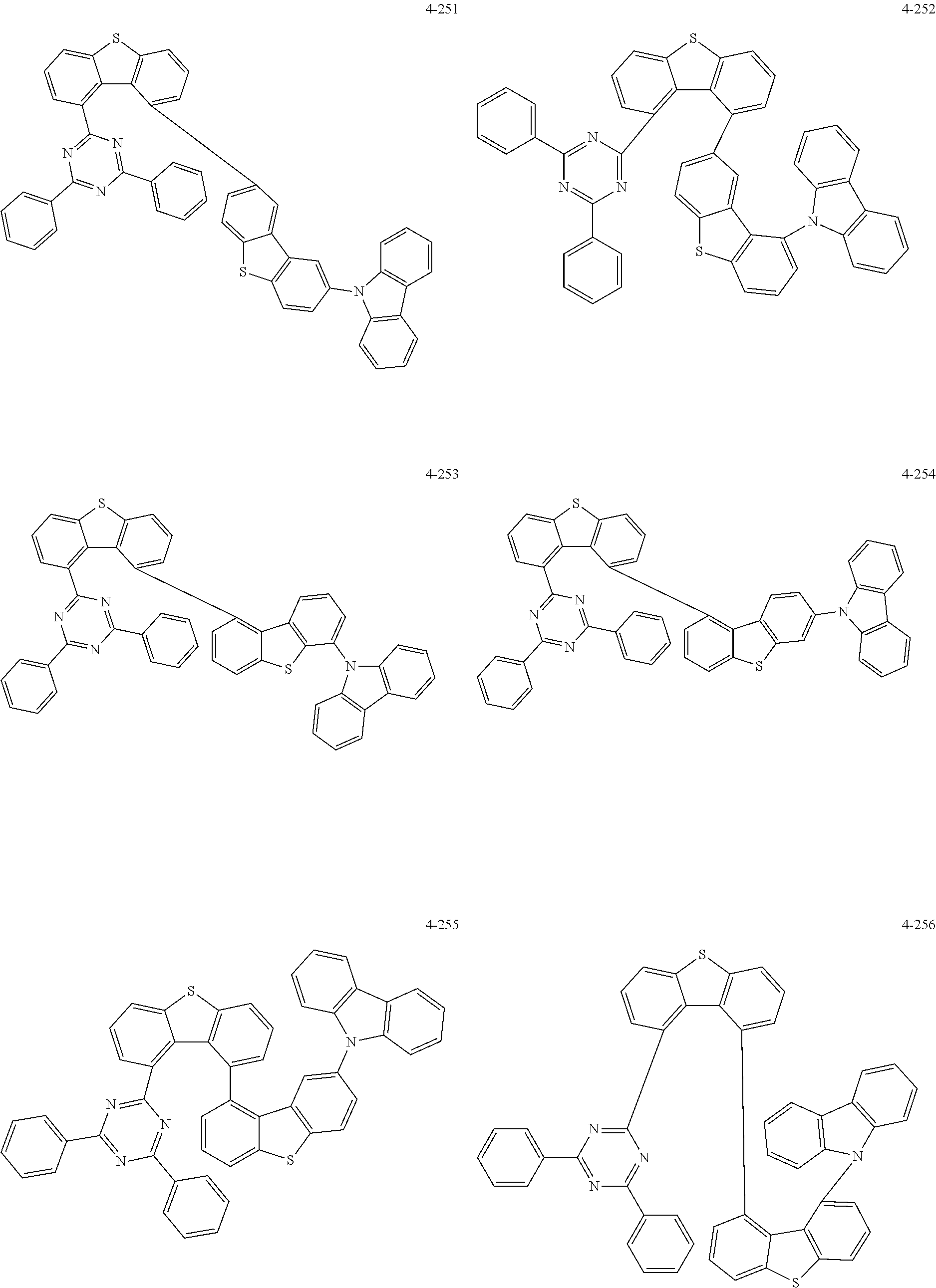

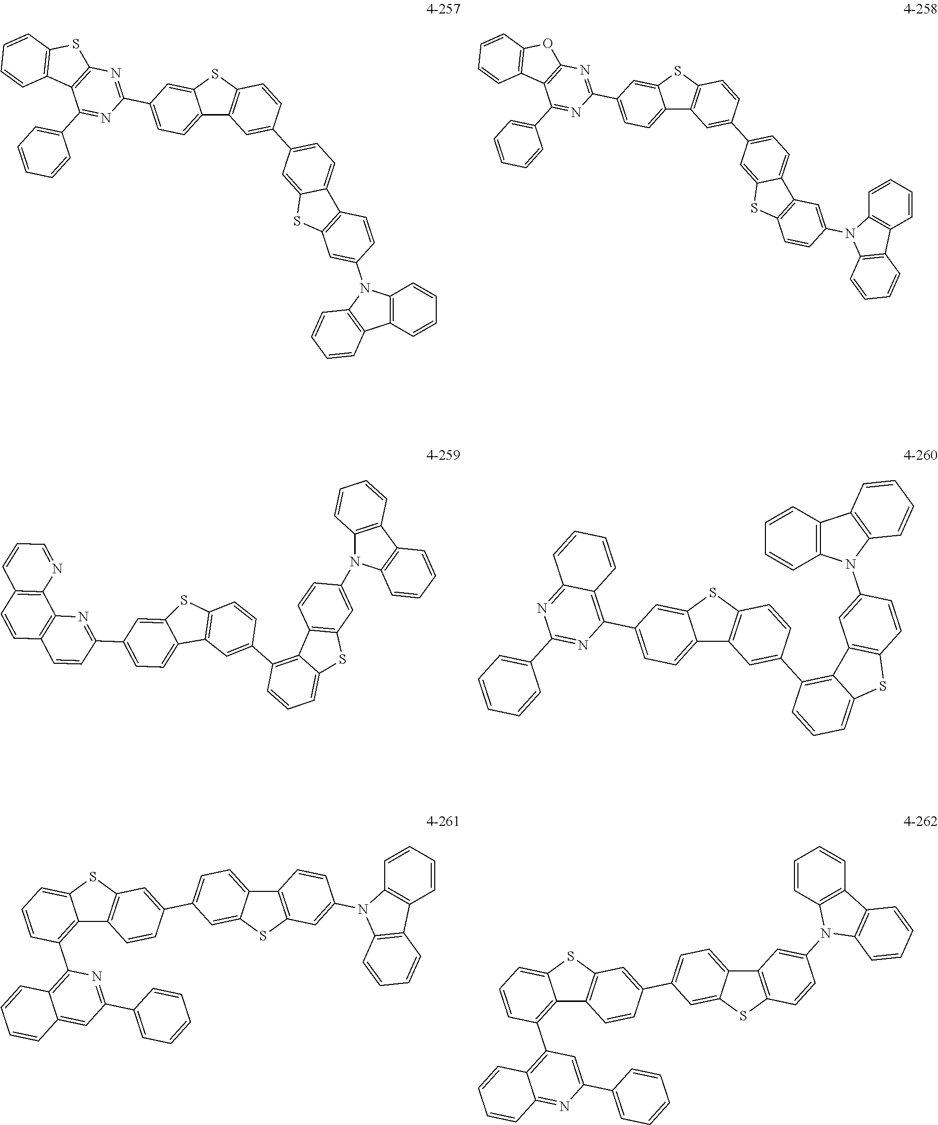

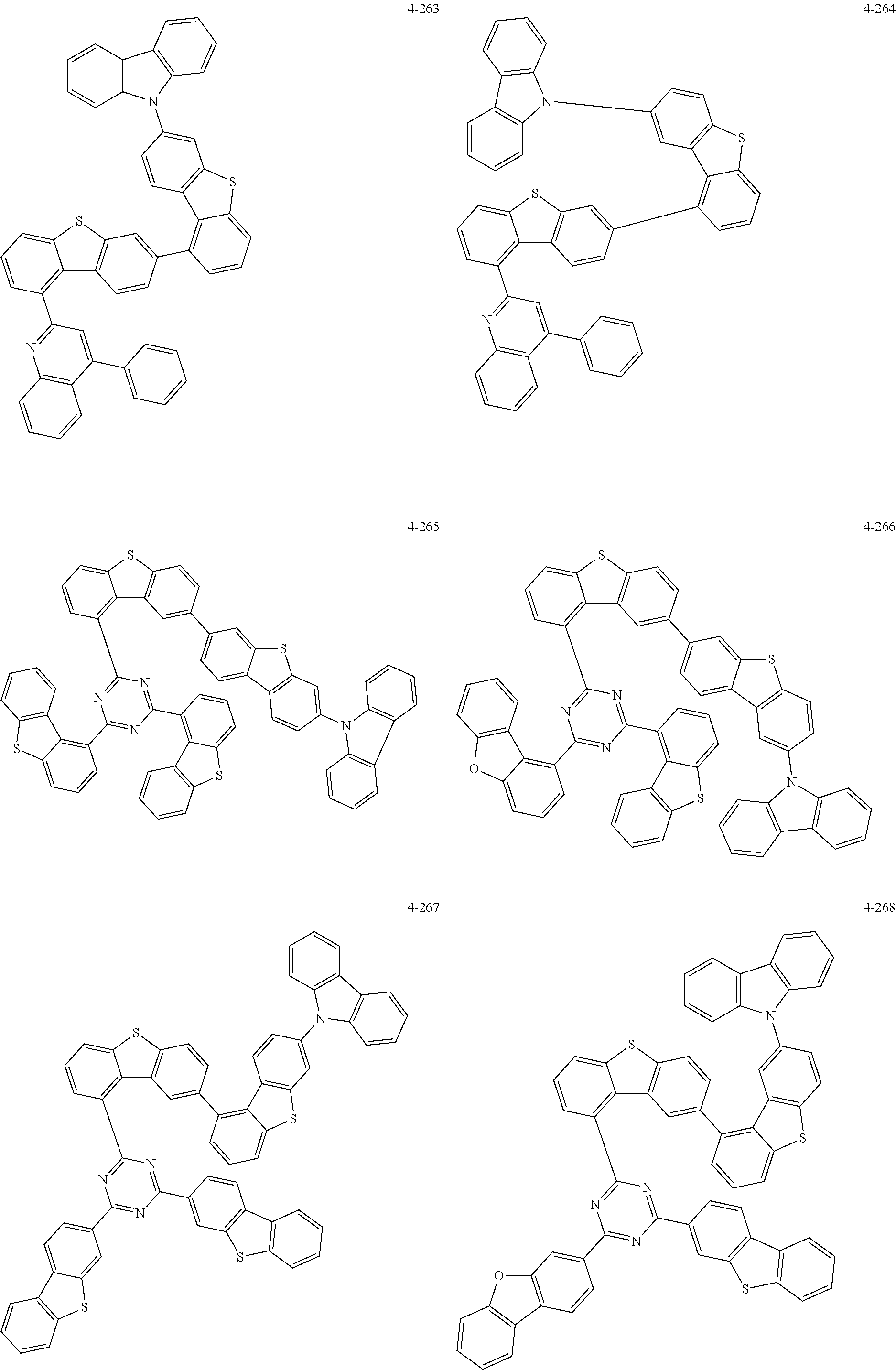

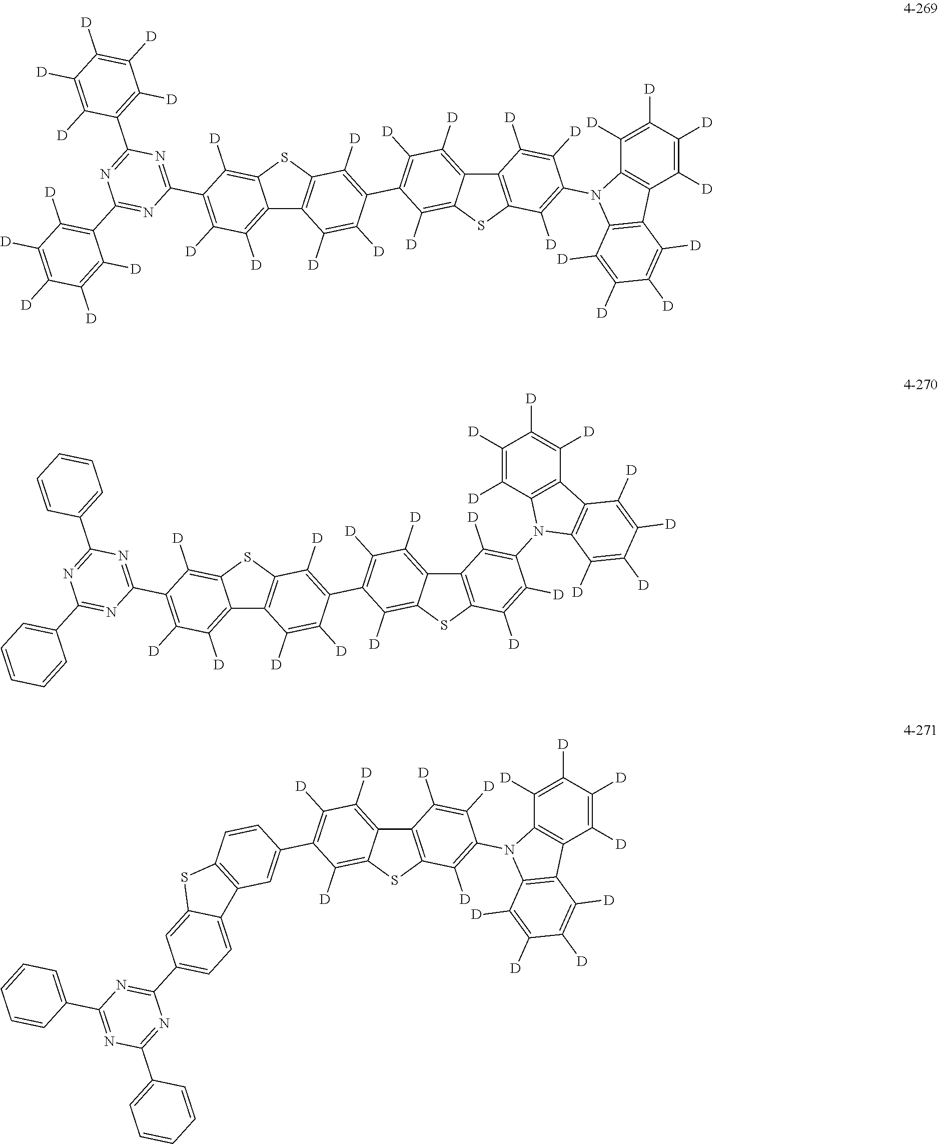

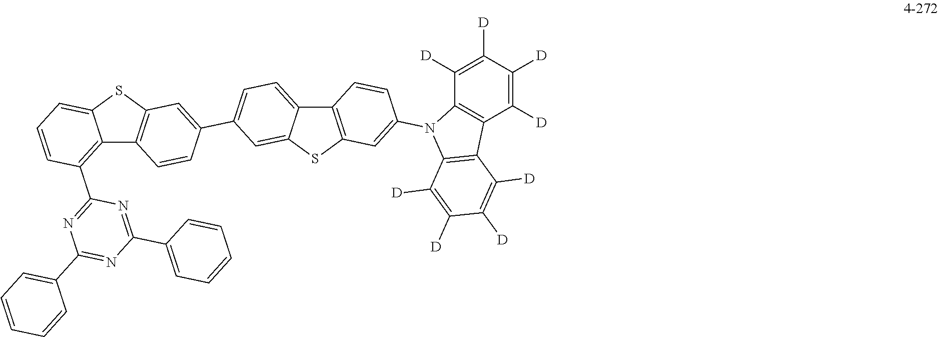 [SMILES: [2H]c1c([2H])c([2H])c2c(c1[2H])c1c([2H])c([2H])c([2H])c([2H])c1n2-c1ccc2c(c1)sc1cc(-c3ccc4c(c3)sc3cccc(-c5nc(-c6ccccc6)nc(-c6ccccc6)n5)c34)ccc12]

In the compound represented by Chemical Formula 1 of the present application comprising the specific examples, orbital disconnection occurs around N of carbazole, and the HOMO orbital is generally localized to the carbazole. Being localized means that electron cloud is relatively focused on carbazole.

When more widely delocalizing the HOMO orbital as above, hole mobility and stability may increase.

In addition, dibenzofuran or dibenzothiophene of the compound represented by Chemical Formula 1 of the present application comprising the specific examples corresponds to a group affecting the LUMO orbital of the heterocyclic group comprising N.

As a result, the LUMO level of the compound may be adjusted depending on the position of substitution of the heterocyclic group comprising N substituting dibenzofuran or dibenzothiophene. This means that the compound represented by Chemical Formula 1 of the present application may be used as a material of an organic light emitting device, particularly a material of a light emitting layer, and more specifically a host material by properly adjusting the LUMO level depending on dopant and electron transfer layer (ETL) materials in the organic light emitting device. In other words, it means that, when dopant and electron transfer layer materials change, a host material needs to be changed depending on the charge balance.

Specifically, when using a compound with a low LUMO level as a material of a light emitting layer, electrons may be readily injected from a host to a dopant, and when using a compound with a high LUMO level as a material of a light emitting layer, electrons may be readily injected from an electron transfer layer (ETL) to a host.

In addition, dibenzofuran or dibenzothiophene substituted with amine or carbazole as in the compound represented by Chemical Formula 1 of the present application comprising the specific examples may be affected by the LUMO orbital widely spread in the heterocyclic group comprising N to have an influence on enhancement in the electron mobility and stability.

In addition, when the compound represented by Chemical Formula 1 of the present application comprising the specific examples has a disconnected structure depending on the position of substitution of amine or carbazole, the HOMO orbital of carbazole or amine is affected, and the HOMO level may be adjusted. This means that the compound represented by Chemical Formula 1 of the present application may be used as a material of an organic light emitting device, particularly a material of a light emitting layer, and more specifically a host material by properly adjusting the HOMO level depending on dopant and electron transfer layer (ETL) materials. In other words, it means that, when dopant and electron transfer layer materials change, a host material needs to be changed depending on the charge balance.

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the heterocyclic compound has a high glass transition temperature (Tg), and has excellent thermal stability. Such an increase in the thermal stability becomes an important factor providing driving stability to a device.

The heterocyclic compound according to one embodiment of the present application may be prepared using a multi-step chemical reaction. Some intermediate compounds are prepared first, and from the intermediate compounds, the heterocyclic compound of Chemical Formula 1 may be prepared. More specifically, the heterocyclic compound according to one embodiment of the present application may be prepared based on preparation examples to describe later.

Another embodiment of the present application provides an organic light emitting device comprising the heterocyclic compound represented by Chemical Formula 1. The “organic light emitting device” may be expressed in terms such as an “organic light emitting diode”, an “OLED”, an “OLED device” and an “organic electroluminescent device”.

The heterocyclic compound may be formed into an organic material layer using a solution coating method as well as a vacuum deposition method when manufacturing the organic light emitting device. Herein, the solution coating method means spin coating, dip coating, inkjet printing, screen printing, a spray method, roll coating and the like, but is not limited thereto.

Specifically, the organic light emitting device according to one embodiment of the present application comprises a first electrode, a second electrode, and one or more organic material layers provided between the first electrode and the second electrode, and one or more layers of the organic material layers comprise the heterocyclic compound represented by Chemical Formula 1. When comprising the heterocyclic compound represented by Chemical Formula 1 in the organic material layer, the organic light emitting device has superior light emission efficiency and lifetime.

In one embodiment of the present application, the first electrode may be an anode, and the second electrode may be a cathode.

In another embodiment, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound according to Chemical Formula 1 may be used as a material of the green organic light emitting device.

In one embodiment of the present application, the organic light emitting device may be a white organic light emitting device, and the heterocyclic compound according to Chemical Formula 1 may be used as a material of the white organic light emitting device.

In one embodiment of the present application, the organic light emitting device may be a red organic light emitting device, and the heterocyclic compound according to Chemical Formula 1 may be used as a material of the red organic light emitting device.

In addition, the organic material layer comprises one or more light emitting layers, and the light emitting layer comprises the heterocyclic compound represented by Chemical Formula 1. When comprising the heterocyclic compound represented by Chemical Formula 1 in the light emitting layer among the organic material layers, the organic light emitting device has more superior light emission efficiency and lifetime.

In addition, in the organic light emitting device of the present application, the organic material layer comprises the heterocyclic compound represented by Chemical Formula 1 as a first compound, and may further comprise a heterocyclic compound represented by the following Chemical Formula 13 or 14 as a second compound.

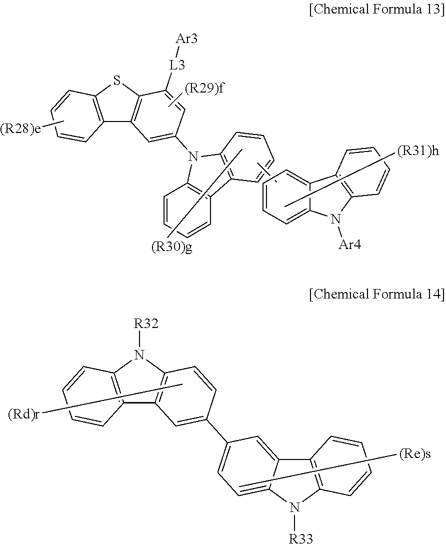

In Chemical Formulae 13 and 14,

L3 is a direct bond; a substituted or unsubstituted arylene group having 6 to 60 carbon atoms; or a substituted or unsubstituted heteroarylene group having 2 to 60 carbon atoms;

R28 to R31, Rd and Re are the same as or different from each other, and each independently selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 2 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more groups adjacent to each other bond to each other to form a substituted or unsubstituted aromatic hydrocarbon ring having 6 to 60 carbon atoms or a substituted or unsubstituted heteroring having 2 to 60 carbon atoms, r and s are an integer of 0 to 7, and when r is 2 or greater, Rds are the same as or different from each other, when s is 2 or greater, Res are the same as or different from each other, e is an integer of 0 to 4, g and h are each an integer of 0 to 7, f is an integer of 0 to 2, and when e is 2 or greater, R28s are the same as or different from each other, when f is an integer of 2, R29s are the same as or different from each other, when g is 2 or greater, R30s are the same as or different from each other, and when h is 2 or greater, R31s are the same as or different from each other,

Ar3 is a substituted or unsubstituted aryl group having 6 to 60 carbon atoms; or a substituted or unsubstituted heteroaryl group having 2 to 60 carbon atoms,

Ar4 is a substituted or unsubstituted aryl group having 6 to 60 carbon atoms; or a substituted or unsubstituted heteroaryl group having 2 to 60 carbon atoms, and

R32 and R33 are the same as or different from each other, and each independently a substituted or unsubstituted silyl group; a substituted or unsubstituted aryl group having 6 to 60 carbon atoms; or a substituted or unsubstituted heteroaryl group having 2 to 60 carbon atoms.

In one embodiment of the present application, the heterocyclic compound represented by Chemical Formula 13 may have a deuterium content of 0% to 100%.

In one embodiment of the present application, the heterocyclic compound represented by Chemical Formula 13 may have a deuterium content of greater than 10% and less than or equal to 100%.

In one embodiment of the present application, the heterocyclic compound represented by Chemical Formula 14 may have a deuterium content of 0% to 100%.

In one embodiment of the present application, the heterocyclic compound represented by Chemical Formula 14 may have a deuterium content of greater than 10% and less than or equal to 100%.

In one embodiment of the present application, L3 may be a direct bond; a substituted or unsubstituted arylene group having 6 to 40 carbon atoms; or a substituted or unsubstituted heteroarylene group having 2 to 40 carbon atoms.

In one embodiment of the present application, L3 may be a direct bond; a substituted or unsubstituted arylene group having 6 to 20 carbon atoms; or a substituted or unsubstituted heteroarylene group having 2 to 20 carbon atoms.

In another embodiment, L3 is a direct bond; or a substituted or unsubstituted phenylene group.

In one embodiment of the present application, R28 to R31 are hydrogen.

In one embodiment of the present application, R28 to R31 are deuterium.

In one embodiment of the present application, Ar3 may be a substituted or unsubstituted aryl group having 6 to 60 carbon atoms; or a substituted or unsubstituted heteroaryl group having 2 to 60 carbon atoms.

In one embodiment of the present application, Ar3 may be a substituted or unsubstituted aryl group having 6 to 40 carbon atoms; or a substituted or unsubstituted heteroaryl group having 2 to 40 carbon atoms.

In one embodiment of the present application, Ar3 may be a substituted or unsubstituted aryl group having 6 to 20 carbon atoms; or a substituted or unsubstituted heteroaryl group having 2 to 20 carbon atoms.

In another embodiment, Ar3 may be a substituted or unsubstituted phenyl group; a substituted or unsubstituted biphenyl group; a substituted or unsubstituted fluorene group; a substituted or unsubstituted dibenzofuran group; or a substituted or unsubstituted dibenzothiophene group.

In another embodiment, Ar3 may be a phenyl group; a biphenyl group; a fluorene group substituted with one or more selected from the group consisting of an alkyl group having 1 to 10 carbon atoms; a dibenzofuran group; or a dibenzothiophene group.

In one embodiment of the present application, Ar4 may be a substituted or unsubstituted aryl group having 6 to 60 carbon atoms; or a substituted or unsubstituted heteroaryl group having 2 to 60 carbon atoms.

In one embodiment of the present application, Ar4 may be a substituted or unsubstituted aryl group having 6 to 40 carbon atoms; or a substituted or unsubstituted heteroaryl group having 2 to 40 carbon atoms.

In one embodiment of the present application, Ar4 may be a substituted or unsubstituted aryl group having 6 to 20 carbon atoms; or a substituted or unsubstituted heteroaryl group having 2 to 20 carbon atoms.

In another embodiment, Ar4 is a substituted or unsubstituted phenyl group.

In another embodiment, Ar4 is a phenyl group.

According to one embodiment of the present application, Chemical Formula 13 may be represented by any one of the following compounds, but is not limited thereto.

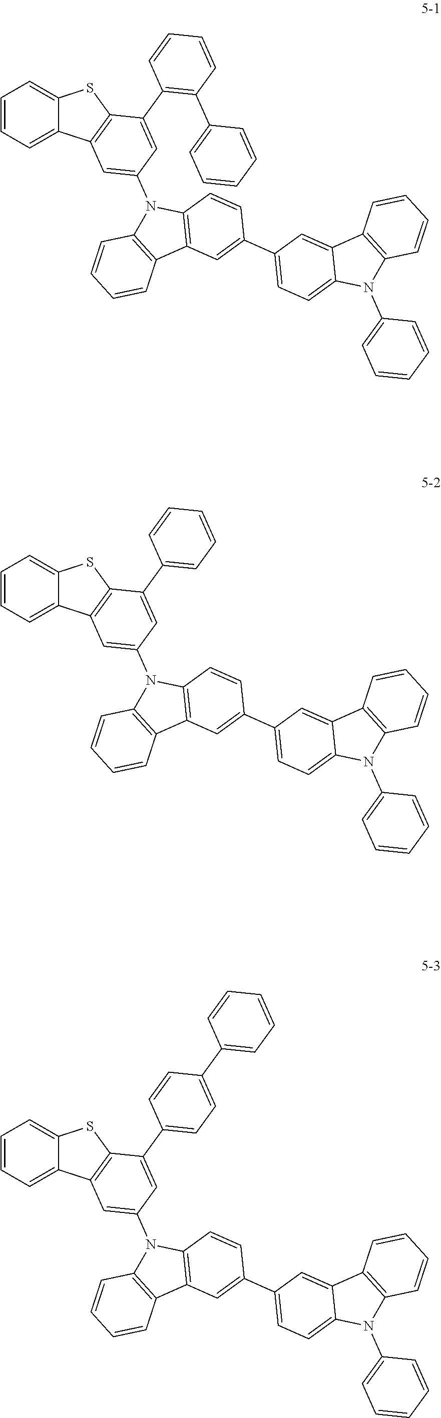 [SMILES: c1ccc(-c2ccccc2-c2cc(-n3c4ccccc4c4cc(-c5ccc6c(c5)c5ccccc5n6-c5ccccc5)ccc43)cc3c2sc2ccccc23)cc1 ; c1ccc(-c2cc(-n3c4ccccc4c4cc(-c5ccc6c(c5)c5ccccc5n6-c5ccccc5)ccc43)cc3c2sc2ccccc23)cc1 ; c1ccc(-c2ccc(-c3cc(-n4c5ccccc5c5cc(-c6ccc7c(c6)c6ccccc6n7-c6ccccc6)ccc54)cc4c3sc3ccccc34)cc2)cc1]

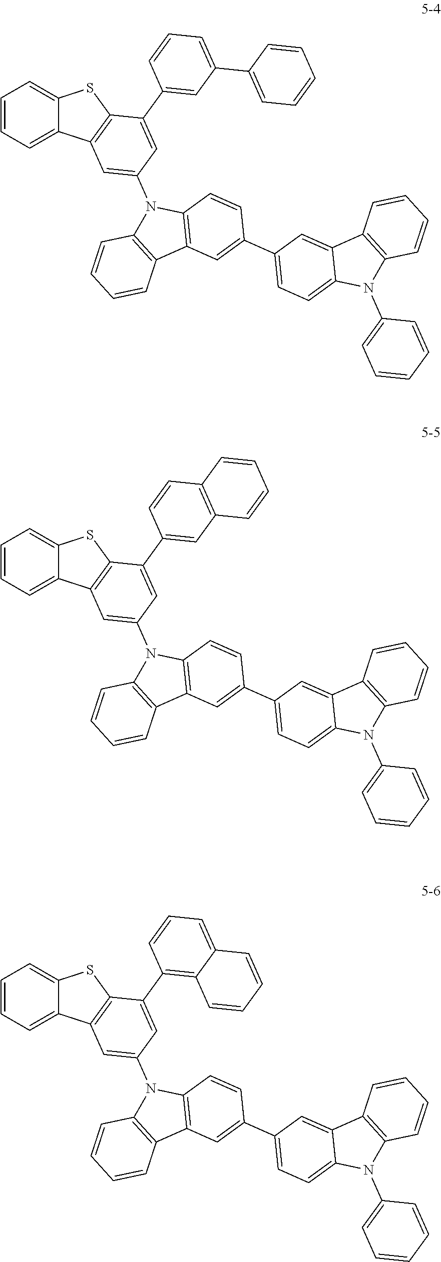 [SMILES: c1ccc(-c2cccc(-c3cc(-n4c5ccccc5c5cc(-c6ccc7c(c6)c6ccccc6n7-c6ccccc6)ccc54)cc4c3sc3ccccc34)c2)cc1 ; c1ccc(-n2c3ccccc3c3cc(-c4ccc5c(c4)c4ccccc4n5-c4cc(-c5ccc6ccccc6c5)c5sc6ccccc6c5c4)ccc32)cc1 ; c1ccc(-n2c3ccccc3c3cc(-c4ccc5c(c4)c4ccccc4n5-c4cc(-c5cccc6ccccc56)c5sc6ccccc6c5c4)ccc32)cc1]

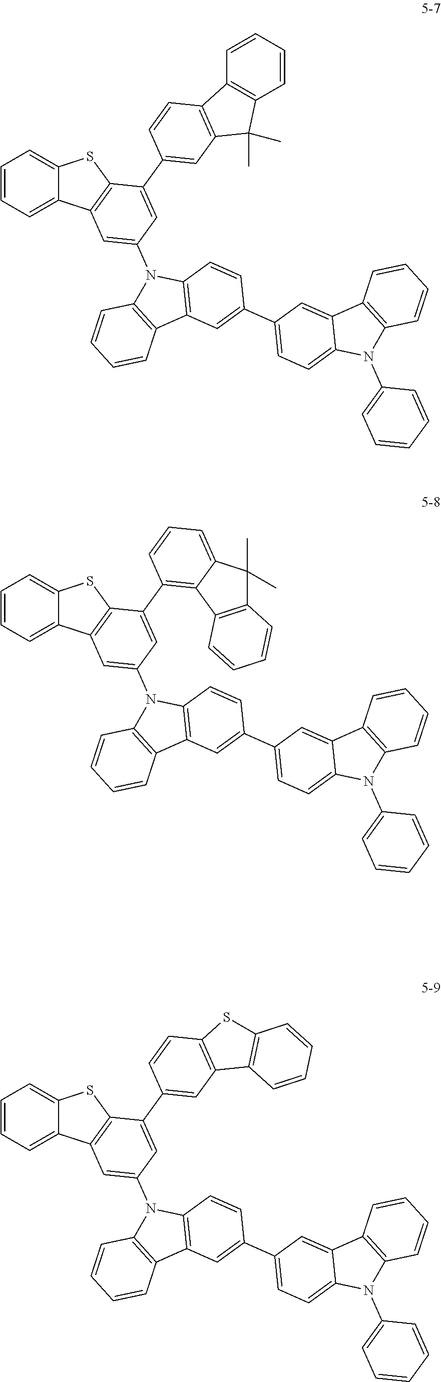 [SMILES: CC1(C)c2ccccc2-c2ccc(-c3cc(-n4c5ccccc5c5cc(-c6ccc7c(c6)c6ccccc6n7-c6ccccc6)ccc54)cc4c3sc3ccccc34)cc21 ; CC1(C)c2ccccc2-c2c(-c3cc(-n4c5ccccc5c5cc(-c6ccc7c(c6)c6ccccc6n7-c6ccccc6)ccc54)cc4c3sc3ccccc34)cccc21 ; c1ccc(-n2c3ccccc3c3cc(-c4ccc5c(c4)c4ccccc4n5-c4cc(-c5ccc6sc7ccccc7c6c5)c5sc6ccccc6c5c4)ccc32)cc1]

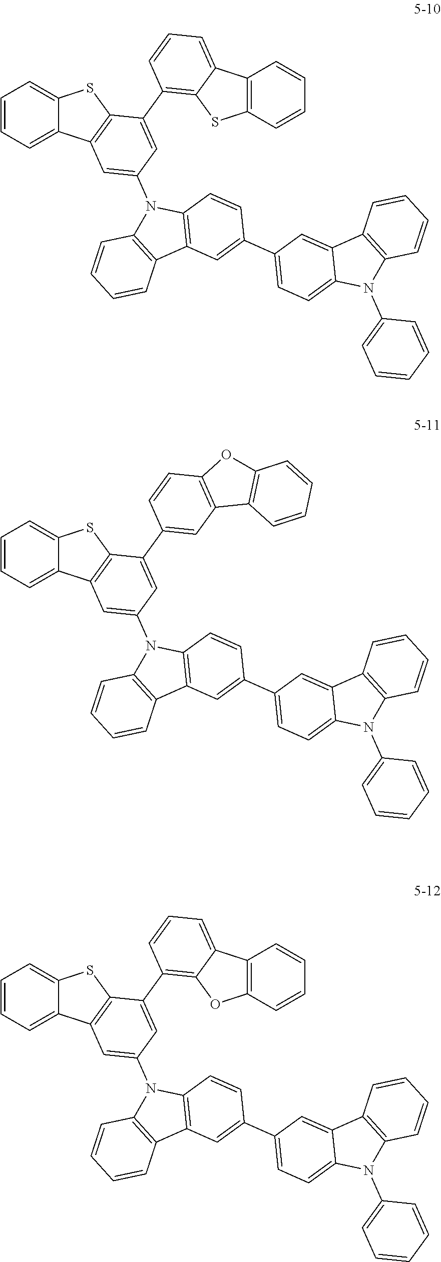 [SMILES: c1ccc(-n2c3ccccc3c3cc(-c4ccc5c(c4)c4ccccc4n5-c4cc(-c5cccc6c5sc5ccccc56)c5sc6ccccc6c5c4)ccc32)cc1 ; c1ccc(-n2c3ccccc3c3cc(-c4ccc5c(c4)c4ccccc4n5-c4cc(-c5ccc6oc7ccccc7c6c5)c5sc6ccccc6c5c4)ccc32)cc1 ; c1ccc(-n2c3ccccc3c3cc(-c4ccc5c(c4)c4ccccc4n5-c4cc(-c5cccc6c5oc5ccccc56)c5sc6ccccc6c5c4)ccc32)cc1]

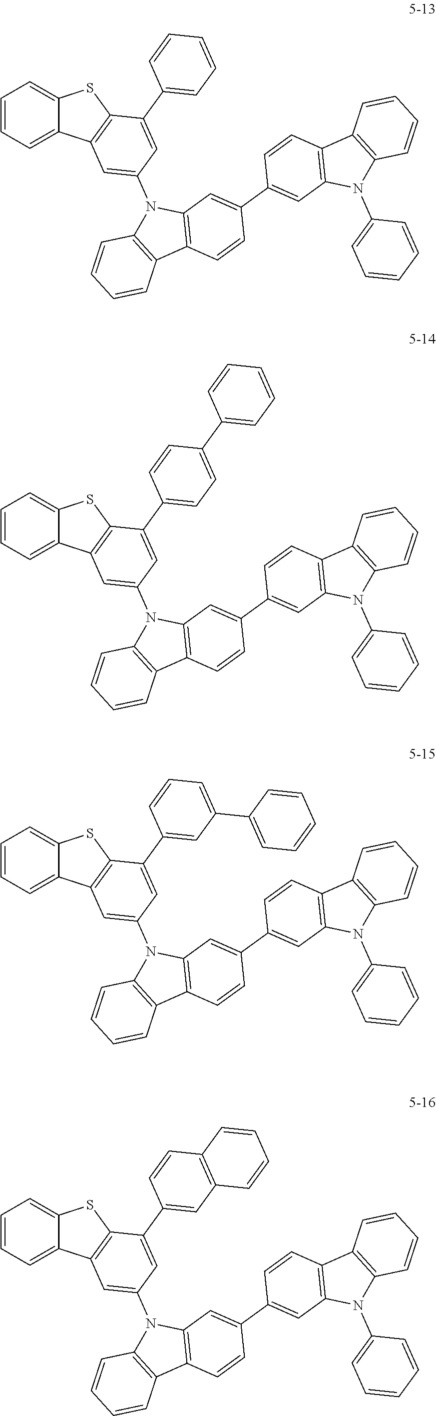 [SMILES: c1ccc(-c2cc(-n3c4ccccc4c4ccc(-c5ccc6c7ccccc7n(-c7ccccc7)c6c5)cc43)cc3c2sc2ccccc23)cc1 ; c1ccc(-c2ccc(-c3cc(-n4c5ccccc5c5ccc(-c6ccc7c8ccccc8n(-c8ccccc8)c7c6)cc54)cc4c3sc3ccccc34)cc2)cc1 ; c1ccc(-c2cccc(-c3cc(-n4c5ccccc5c5ccc(-c6ccc7c8ccccc8n(-c8ccccc8)c7c6)cc54)cc4c3sc3ccccc34)c2)cc1 ; c1ccc(-n2c3ccccc3c3ccc(-c4ccc5c6ccccc6n(-c6cc(-c7ccc8ccccc8c7)c7sc8ccccc8c7c6)c5c4)cc32)cc1]

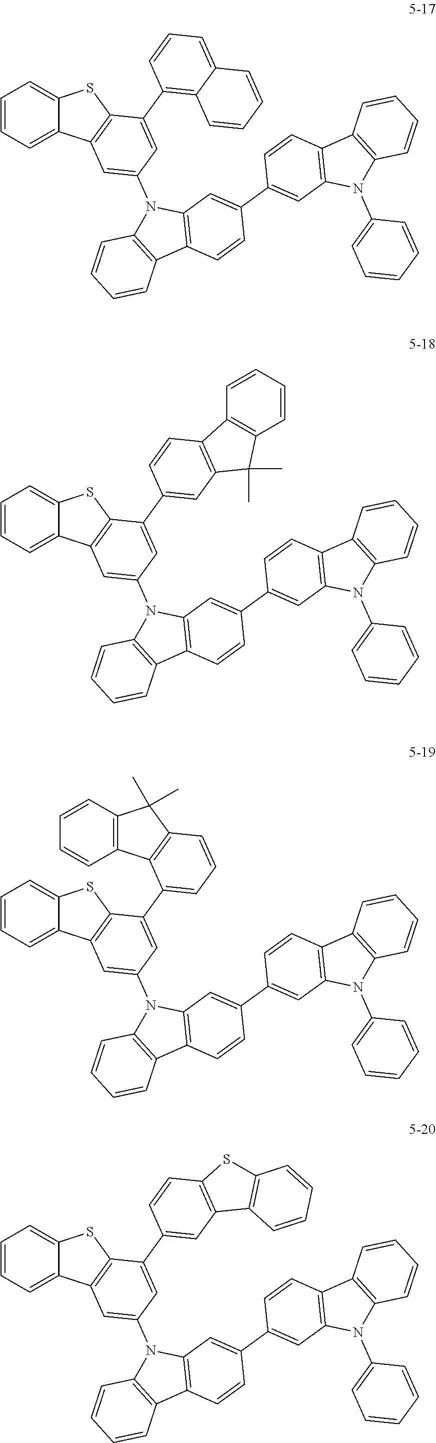 [SMILES: c1ccc(-n2c3ccccc3c3ccc(-c4ccc5c6ccccc6n(-c6cc(-c7cccc8ccccc78)c7sc8ccccc8c7c6)c5c4)cc32)cc1 ; CC1(C)c2ccccc2-c2ccc(-c3cc(-n4c5ccccc5c5ccc(-c6ccc7c8ccccc8n(-c8ccccc8)c7c6)cc54)cc4c3sc3ccccc34)cc21 ; CC1(C)c2ccccc2-c2c(-c3cc(-n4c5ccccc5c5ccc(-c6ccc7c8ccccc8n(-c8ccccc8)c7c6)cc54)cc4c3sc3ccccc34)cccc21 ; c1ccc(-n2c3ccccc3c3ccc(-c4ccc5c6ccccc6n(-c6cc(-c7ccc8sc9ccccc9c8c7)c7sc8ccccc8c7c6)c5c4)cc32)cc1]

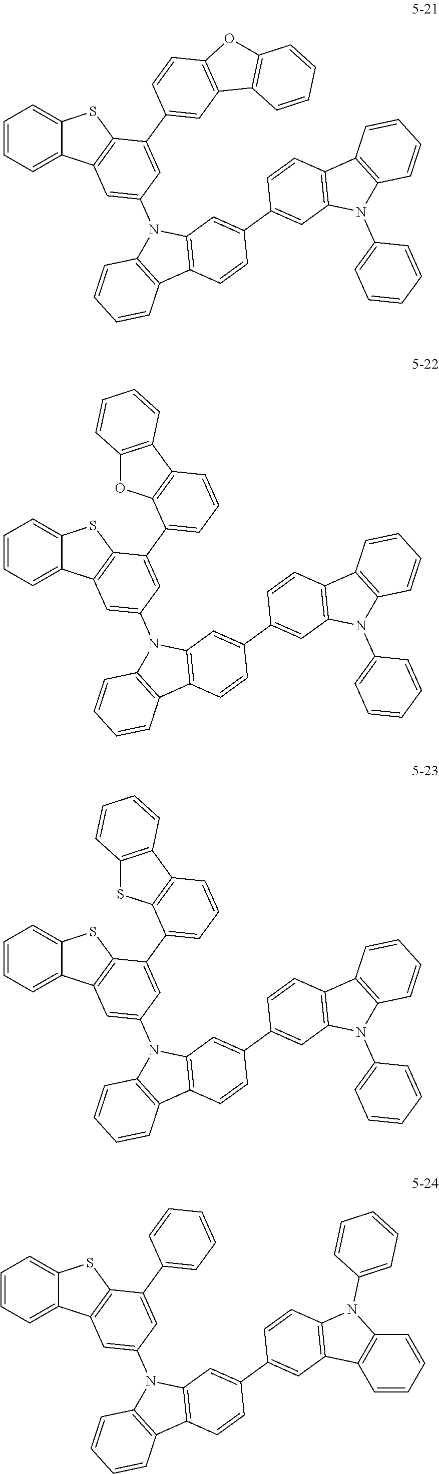 [SMILES: c1ccc(-n2c3ccccc3c3ccc(-c4ccc5c6ccccc6n(-c6cc(-c7ccc8oc9ccccc9c8c7)c7sc8ccccc8c7c6)c5c4)cc32)cc1 ; c1ccc(-n2c3ccccc3c3ccc(-c4ccc5c6ccccc6n(-c6cc(-c7cccc8c7oc7ccccc78)c7sc8ccccc8c7c6)c5c4)cc32)cc1 ; c1ccc(-n2c3ccccc3c3ccc(-c4ccc5c6ccccc6n(-c6cc(-c7cccc8c7sc7ccccc78)c7sc8ccccc8c7c6)c5c4)cc32)cc1 ; c1ccc(-c2cc(-n3c4ccccc4c4ccc(-c5ccc6c(c5)c5ccccc5n6-c5ccccc5)cc43)cc3c2sc2ccccc23)cc1]

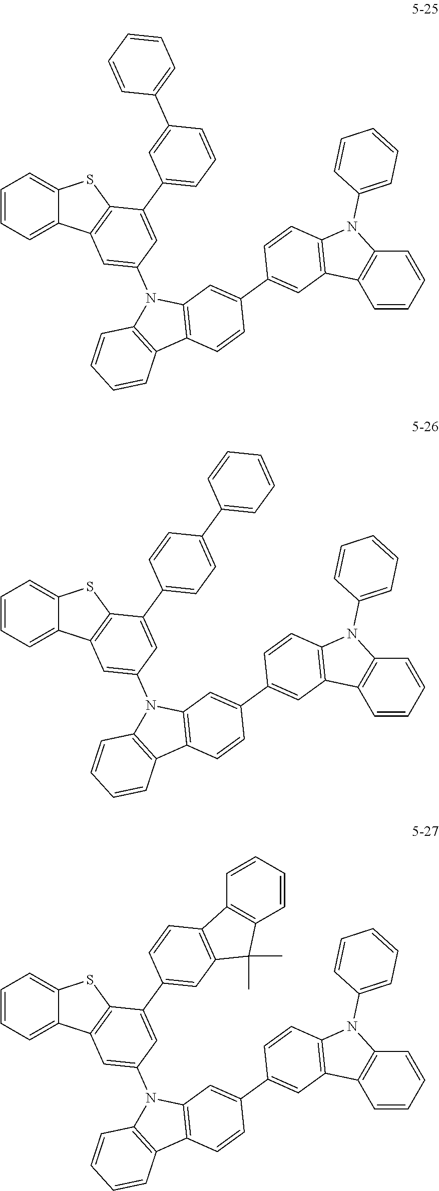 [SMILES: c1ccc(-c2cccc(-c3cc(-n4c5ccccc5c5ccc(-c6ccc7c(c6)c6ccccc6n7-c6ccccc6)cc54)cc4c3sc3ccccc34)c2)cc1 ; c1ccc(-c2ccc(-c3cc(-n4c5ccccc5c5ccc(-c6ccc7c(c6)c6ccccc6n7-c6ccccc6)cc54)cc4c3sc3ccccc34)cc2)cc1 ; CC1(C)c2ccccc2-c2ccc(-c3cc(-n4c5ccccc5c5ccc(-c6ccc7c(c6)c6ccccc6n7-c6ccccc6)cc54)cc4c3sc3ccccc34)cc21]

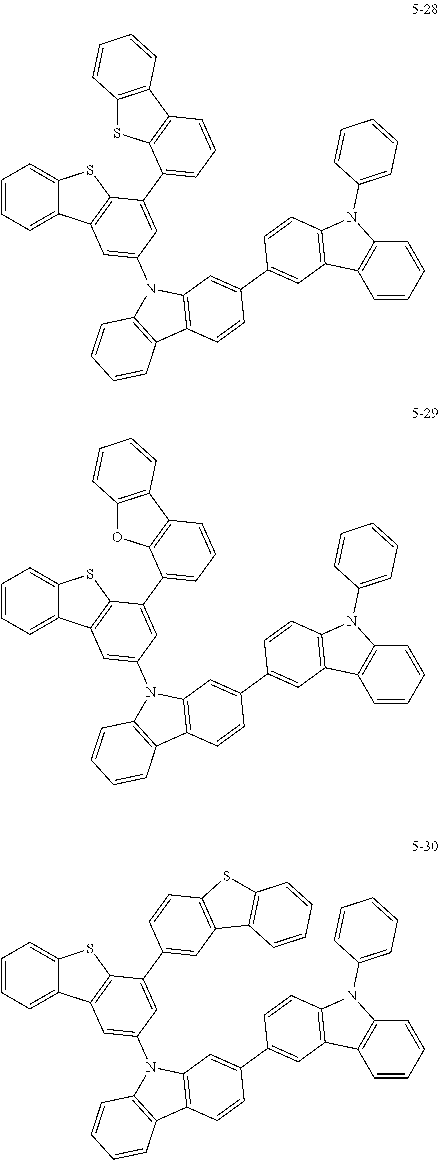 [SMILES: c1ccc(-n2c3ccccc3c3cc(-c4ccc5c6ccccc6n(-c6cc(-c7cccc8c7sc7ccccc78)c7sc8ccccc8c7c6)c5c4)ccc32)cc1 ; c1ccc(-n2c3ccccc3c3cc(-c4ccc5c6ccccc6n(-c6cc(-c7cccc8c7oc7ccccc78)c7sc8ccccc8c7c6)c5c4)ccc32)cc1 ; c1ccc(-n2c3ccccc3c3cc(-c4ccc5c6ccccc6n(-c6cc(-c7ccc8sc9ccccc9c8c7)c7sc8ccccc8c7c6)c5c4)ccc32)cc1]

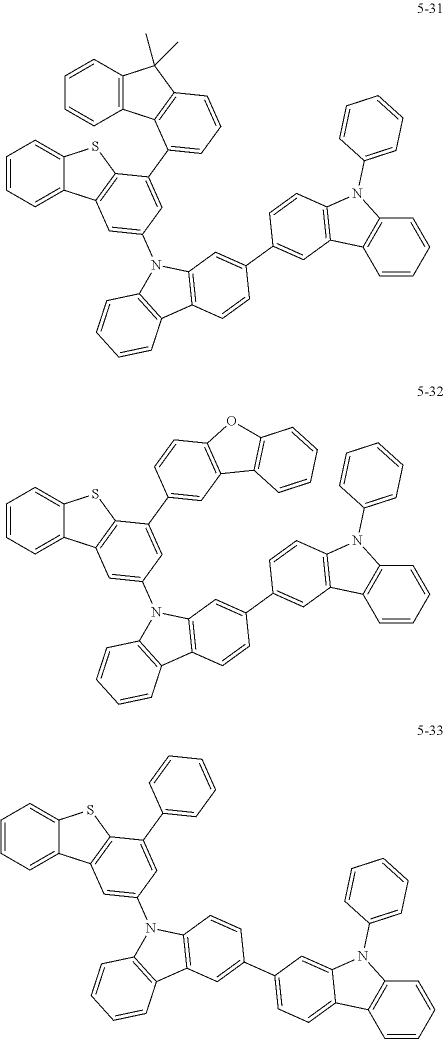 [SMILES: CC1(C)c2ccccc2-c2c(-c3cc(-n4c5ccccc5c5ccc(-c6ccc7c(c6)c6ccccc6n7-c6ccccc6)cc54)cc4c3sc3ccccc34)cccc21 ; c1ccc(-n2c3ccccc3c3cc(-c4ccc5c6ccccc6n(-c6cc(-c7ccc8oc9ccccc9c8c7)c7sc8ccccc8c7c6)c5c4)ccc32)cc1 ; c1ccc(-c2cc(-n3c4ccccc4c4cc(-c5ccc6c7ccccc7n(-c7ccccc7)c6c5)ccc43)cc3c2sc2ccccc23)cc1]

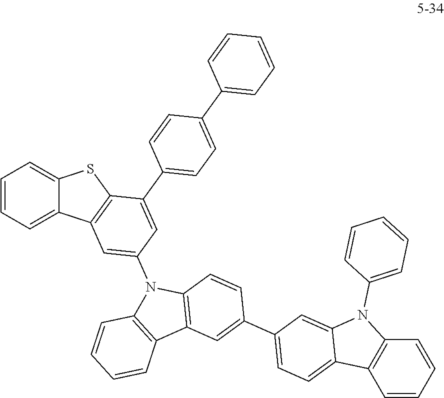 [SMILES: c1ccc(-c2ccc(-c3cc(-n4c5ccccc5c5cc(-c6ccc7c8ccccc8n(-c8ccccc8)c7c6)ccc54)cc4c3sc3ccccc34)cc2)cc1]

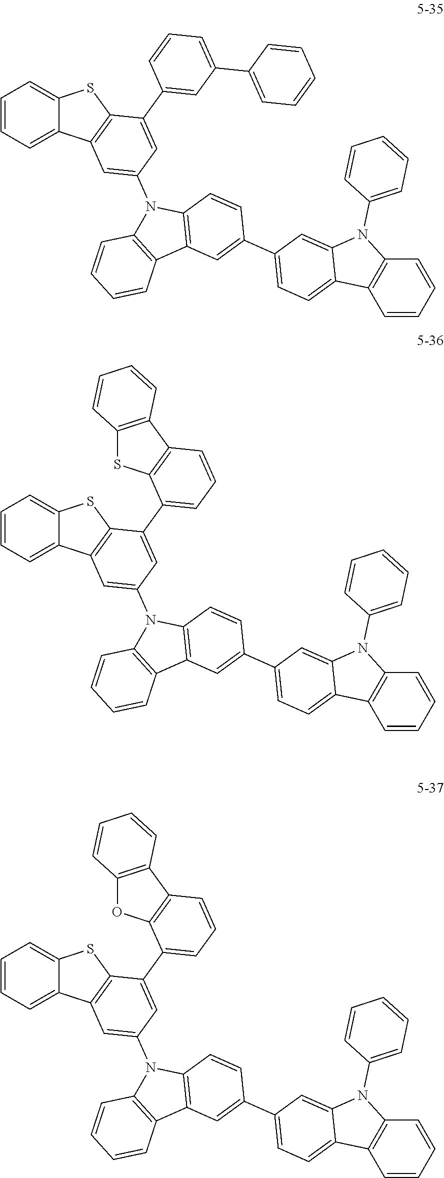 [SMILES: c1ccc(-c2cccc(-c3cc(-n4c5ccccc5c5cc(-c6ccc7c8ccccc8n(-c8ccccc8)c7c6)ccc54)cc4c3sc3ccccc34)c2)cc1 ; c1ccc(-n2c3ccccc3c3ccc(-c4ccc5c(c4)c4ccccc4n5-c4cc(-c5cccc6c5sc5ccccc56)c5sc6ccccc6c5c4)cc32)cc1 ; c1ccc(-n2c3ccccc3c3ccc(-c4ccc5c(c4)c4ccccc4n5-c4cc(-c5cccc6c5oc5ccccc56)c5sc6ccccc6c5c4)cc32)cc1]

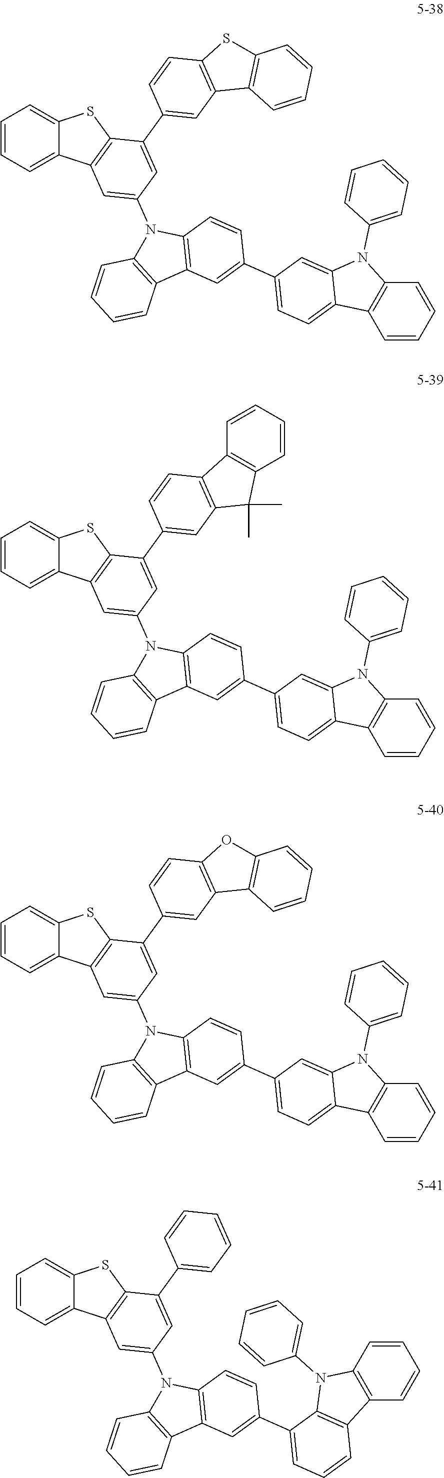 [SMILES: c1ccc(-n2c3ccccc3c3ccc(-c4ccc5c(c4)c4ccccc4n5-c4cc(-c5ccc6sc7ccccc7c6c5)c5sc6ccccc6c5c4)cc32)cc1 ; CC1(C)c2ccccc2-c2ccc(-c3cc(-n4c5ccccc5c5cc(-c6ccc7c8ccccc8n(-c8ccccc8)c7c6)ccc54)cc4c3sc3ccccc34)cc21 ; c1ccc(-n2c3ccccc3c3ccc(-c4ccc5c(c4)c4ccccc4n5-c4cc(-c5ccc6oc7ccccc7c6c5)c5sc6ccccc6c5c4)cc32)cc1 ; c1ccc(-c2cc(-n3c4ccccc4c4cc(-c5cccc6c7ccccc7n(-c7ccccc7)c56)ccc43)cc3c2sc2ccccc23)cc1]

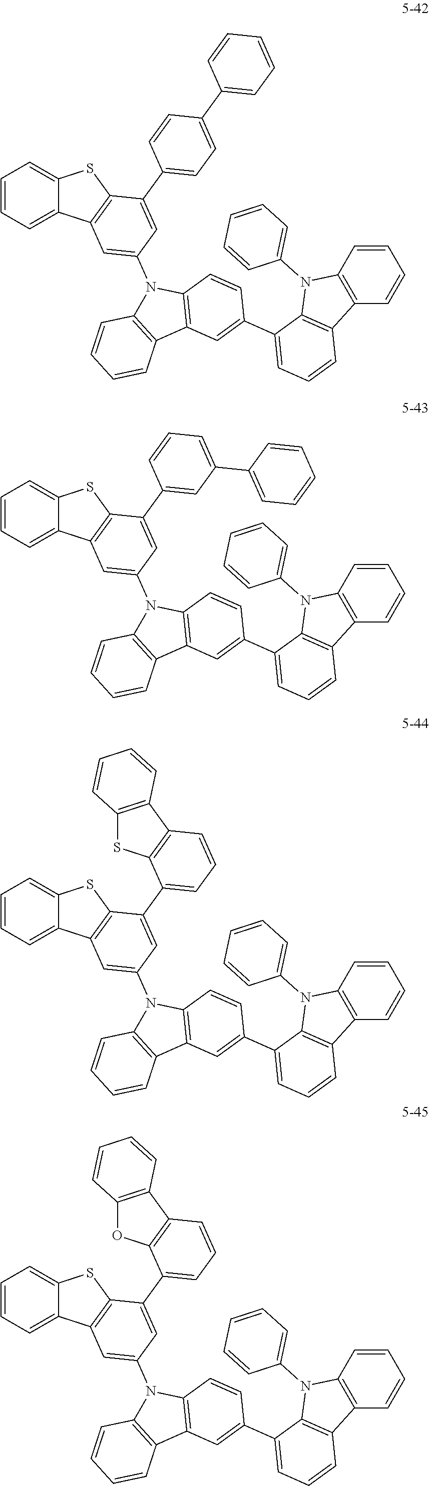

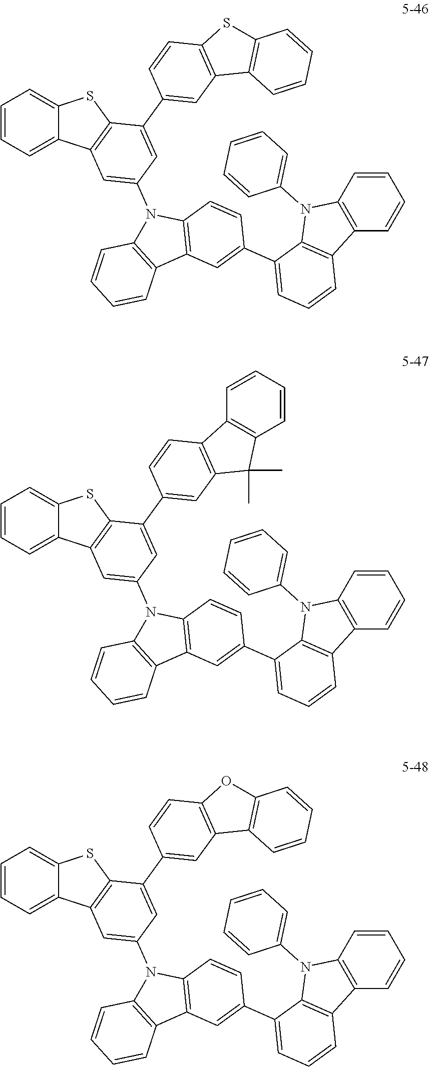 [SMILES: c1ccc(-n2c3ccccc3c3cccc(-c4ccc5c(c4)c4ccccc4n5-c4cc(-c5ccc6sc7ccccc7c6c5)c5sc6ccccc6c5c4)c32)cc1 ; CC1(C)c2ccccc2-c2ccc(-c3cc(-n4c5ccccc5c5cc(-c6cccc7c8ccccc8n(-c8ccccc8)c67)ccc54)cc4c3sc3ccccc34)cc21 ; c1ccc(-n2c3ccccc3c3cccc(-c4ccc5c(c4)c4ccccc4n5-c4cc(-c5ccc6oc7ccccc7c6c5)c5sc6ccccc6c5c4)c32)cc1]

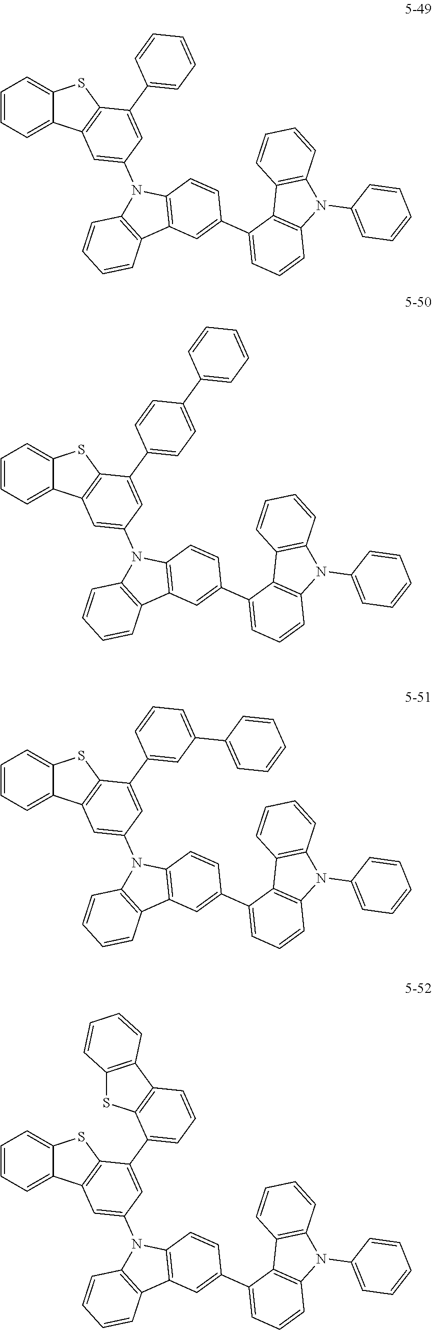 [SMILES: c1ccc(-c2cc(-n3c4ccccc4c4cc(-c5cccc6c5c5ccccc5n6-c5ccccc5)ccc43)cc3c2sc2ccccc23)cc1 ; c1ccc(-c2ccc(-c3cc(-n4c5ccccc5c5cc(-c6cccc7c6c6ccccc6n7-c6ccccc6)ccc54)cc4c3sc3ccccc34)cc2)cc1 ; c1ccc(-c2cccc(-c3cc(-n4c5ccccc5c5cc(-c6cccc7c6c6ccccc6n7-c6ccccc6)ccc54)cc4c3sc3ccccc34)c2)cc1 ; c1ccc(-n2c3ccccc3c3c(-c4ccc5c(c4)c4ccccc4n5-c4cc(-c5cccc6c5sc5ccccc56)c5sc6ccccc6c5c4)cccc32)cc1]

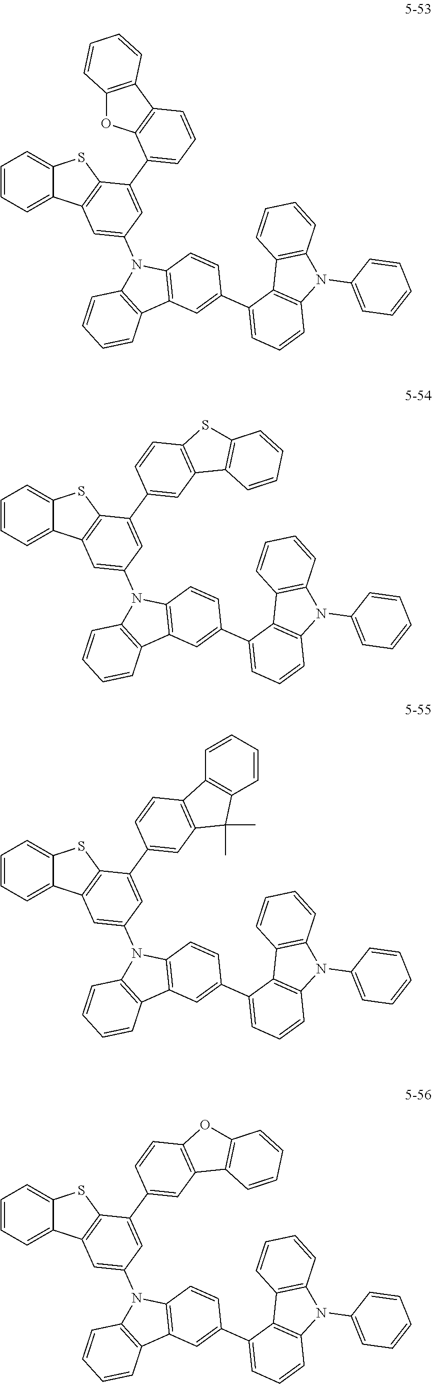 [SMILES: c1ccc(-n2c3ccccc3c3c(-c4ccc5c(c4)c4ccccc4n5-c4cc(-c5cccc6c5oc5ccccc56)c5sc6ccccc6c5c4)cccc32)cc1 ; c1ccc(-n2c3ccccc3c3c(-c4ccc5c(c4)c4ccccc4n5-c4cc(-c5ccc6sc7ccccc7c6c5)c5sc6ccccc6c5c4)cccc32)cc1 ; CC1(C)c2ccccc2-c2ccc(-c3cc(-n4c5ccccc5c5cc(-c6cccc7c6c6ccccc6n7-c6ccccc6)ccc54)cc4c3sc3ccccc34)cc21 ; c1ccc(-n2c3ccccc3c3c(-c4ccc5c(c4)c4ccccc4n5-c4cc(-c5ccc6oc7ccccc7c6c5)c5sc6ccccc6c5c4)cccc32)cc1]

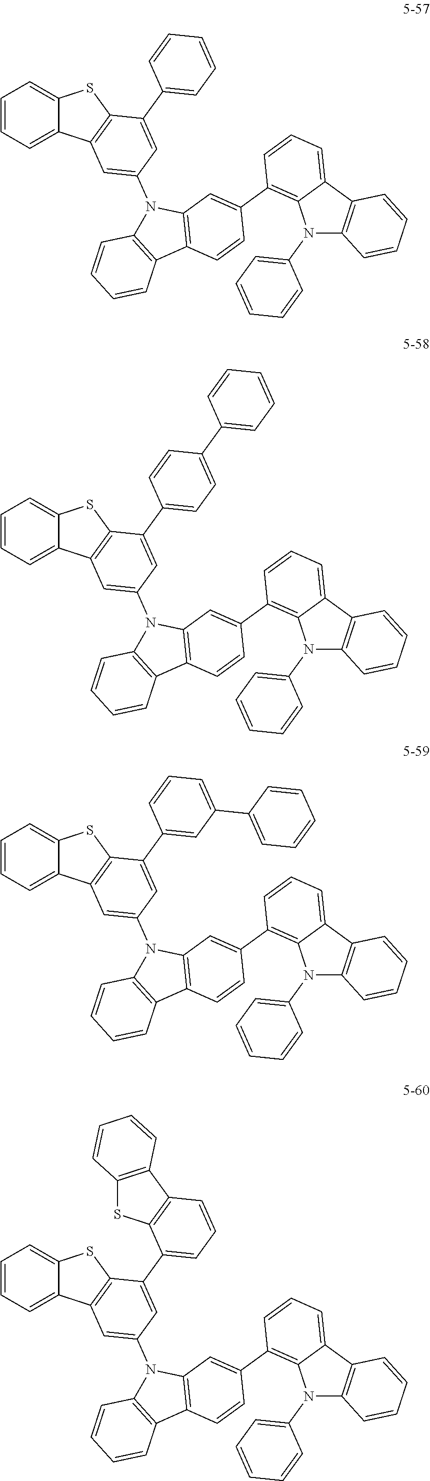 [SMILES: c1ccc(-c2cc(-n3c4ccccc4c4ccc(-c5cccc6c7ccccc7n(-c7ccccc7)c56)cc43)cc3c2sc2ccccc23)cc1 ; c1ccc(-c2ccc(-c3cc(-n4c5ccccc5c5ccc(-c6cccc7c8ccccc8n(-c8ccccc8)c67)cc54)cc4c3sc3ccccc34)cc2)cc1 ; c1ccc(-c2cccc(-c3cc(-n4c5ccccc5c5ccc(-c6cccc7c8ccccc8n(-c8ccccc8)c67)cc54)cc4c3sc3ccccc34)c2)cc1 ; c1ccc(-n2c3ccccc3c3cccc(-c4ccc5c6ccccc6n(-c6cc(-c7cccc8c7sc7ccccc78)c7sc8ccccc8c7c6)c5c4)c32)cc1]

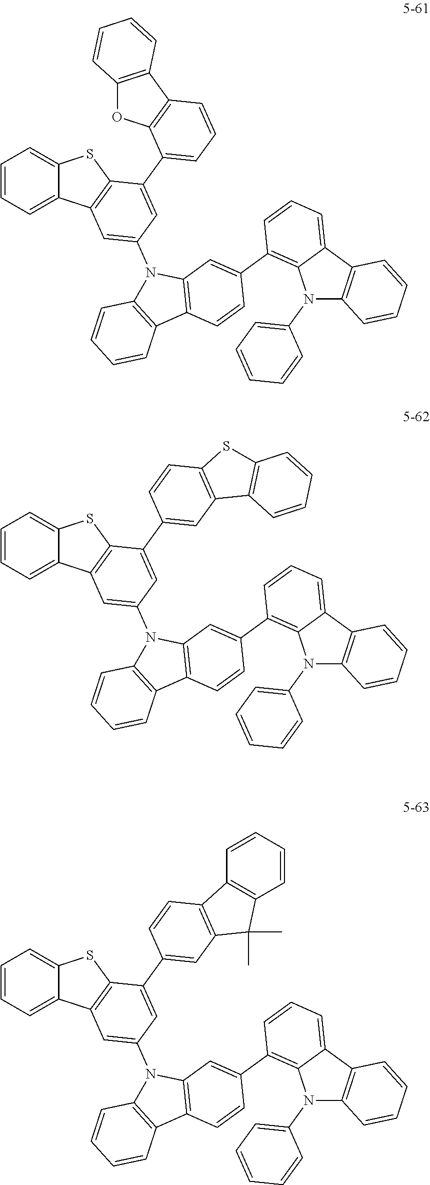 [SMILES: c1ccc(-n2c3ccccc3c3cccc(-c4ccc5c6ccccc6n(-c6cc(-c7cccc8c7oc7ccccc78)c7sc8ccccc8c7c6)c5c4)c32)cc1 ; c1ccc(-n2c3ccccc3c3cccc(-c4ccc5c6ccccc6n(-c6cc(-c7ccc8sc9ccccc9c8c7)c7sc8ccccc8c7c6)c5c4)c32)cc1 ; CC1(C)c2ccccc2-c2ccc(-c3cc(-n4c5ccccc5c5ccc(-c6cccc7c8ccccc8n(-c8ccccc8)c67)cc54)cc4c3sc3ccccc34)cc21]

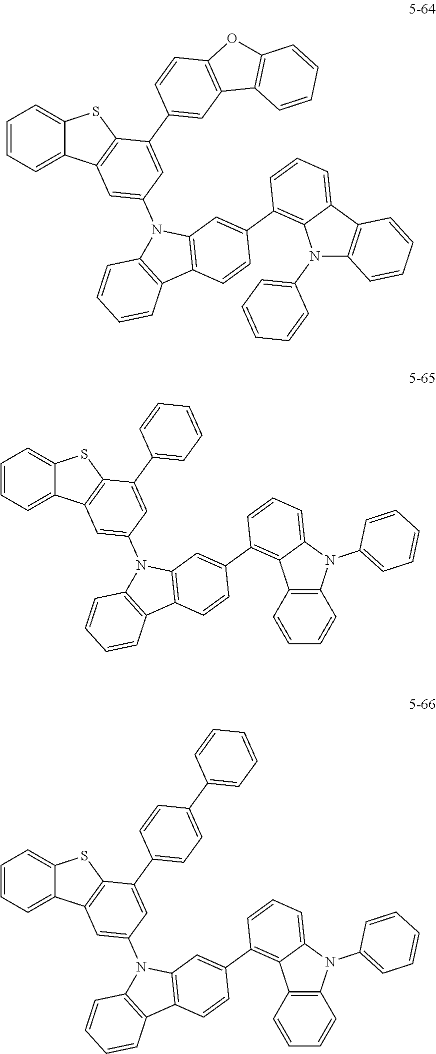 [SMILES: c1ccc(-n2c3ccccc3c3cccc(-c4ccc5c6ccccc6n(-c6cc(-c7ccc8oc9ccccc9c8c7)c7sc8ccccc8c7c6)c5c4)c32)cc1 ; c1ccc(-c2cc(-n3c4ccccc4c4ccc(-c5cccc6c5c5ccccc5n6-c5ccccc5)cc43)cc3c2sc2ccccc23)cc1 ; c1ccc(-c2ccc(-c3cc(-n4c5ccccc5c5ccc(-c6cccc7c6c6ccccc6n7-c6ccccc6)cc54)cc4c3sc3ccccc34)cc2)cc1]

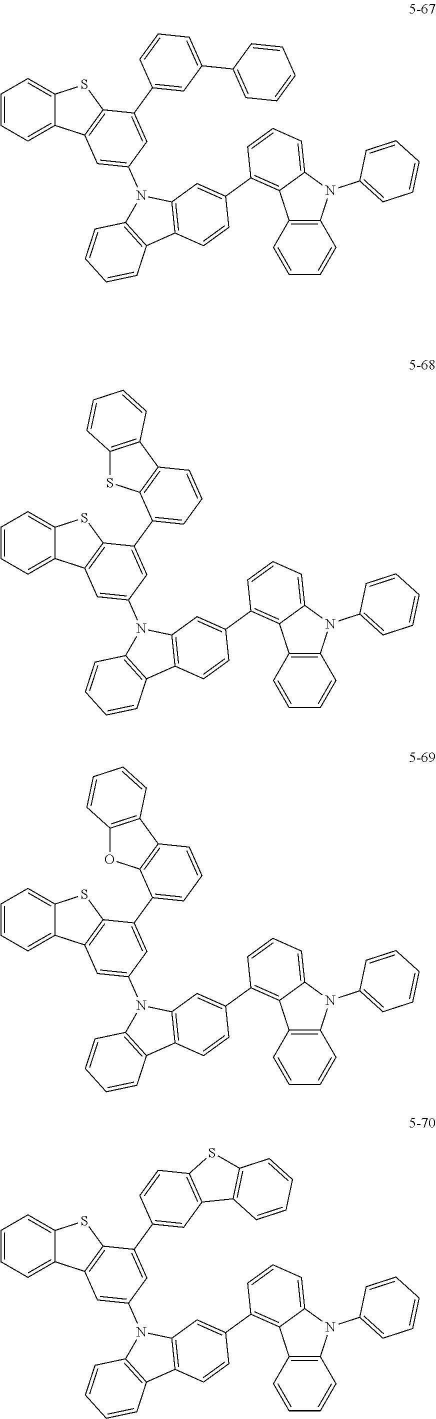 [SMILES: c1ccc(-c2cccc(-c3cc(-n4c5ccccc5c5ccc(-c6cccc7c6c6ccccc6n7-c6ccccc6)cc54)cc4c3sc3ccccc34)c2)cc1 ; c1ccc(-n2c3ccccc3c3c(-c4ccc5c6ccccc6n(-c6cc(-c7cccc8c7sc7ccccc78)c7sc8ccccc8c7c6)c5c4)cccc32)cc1 ; c1ccc(-n2c3ccccc3c3c(-c4ccc5c6ccccc6n(-c6cc(-c7cccc8c7oc7ccccc78)c7sc8ccccc8c7c6)c5c4)cccc32)cc1 ; c1ccc(-n2c3ccccc3c3c(-c4ccc5c6ccccc6n(-c6cc(-c7ccc8sc9ccccc9c8c7)c7sc8ccccc8c7c6)c5c4)cccc32)cc1]

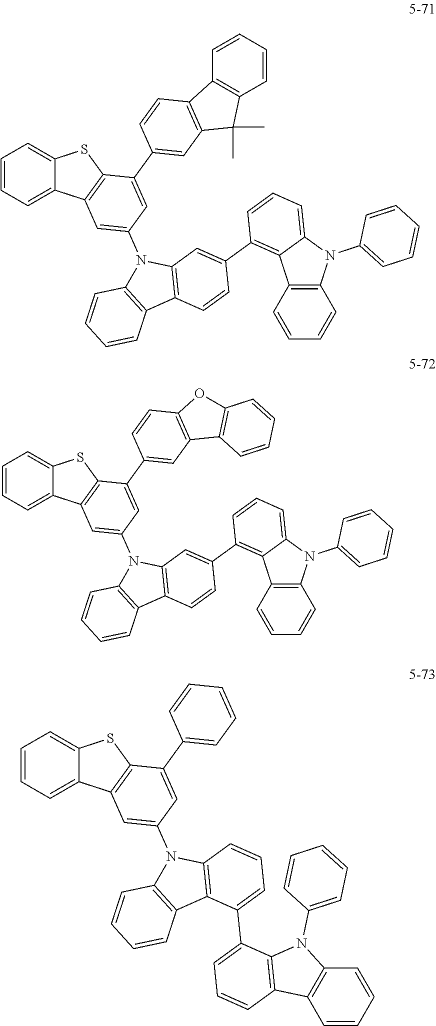 [SMILES: CC1(C)c2ccccc2-c2ccc(-c3cc(-n4c5ccccc5c5ccc(-c6cccc7c6c6ccccc6n7-c6ccccc6)cc54)cc4c3sc3ccccc34)cc21 ; c1ccc(-n2c3ccccc3c3c(-c4ccc5c6ccccc6n(-c6cc(-c7ccc8oc9ccccc9c8c7)c7sc8ccccc8c7c6)c5c4)cccc32)cc1 ; c1ccc(-c2cc(-n3c4ccccc4c4c(-c5cccc6c7ccccc7n(-c7ccccc7)c56)cccc43)cc3c2sc2ccccc23)cc1]

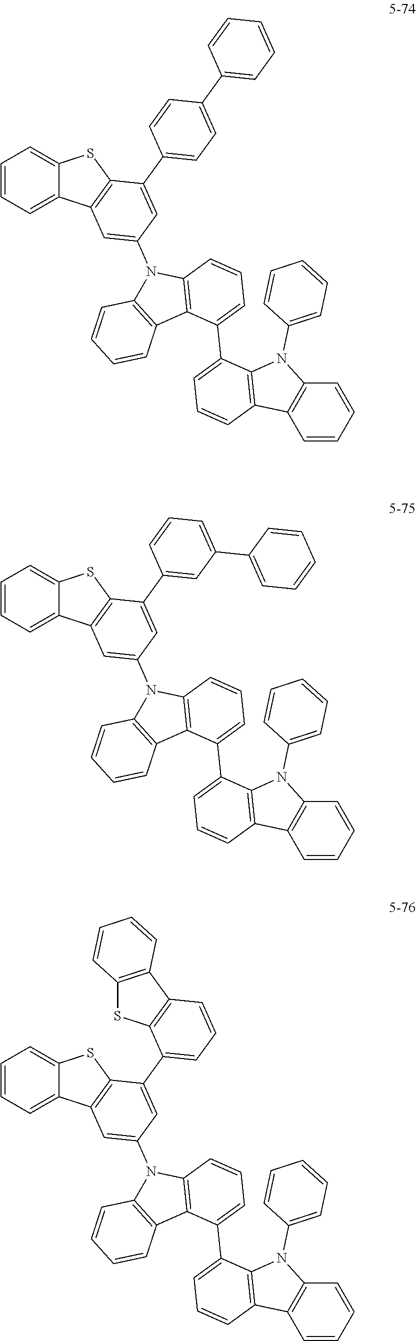 [SMILES: c1ccc(-c2ccc(-c3cc(-n4c5ccccc5c5c(-c6cccc7c8ccccc8n(-c8ccccc8)c67)cccc54)cc4c3sc3ccccc34)cc2)cc1 ; c1ccc(-c2cccc(-c3cc(-n4c5ccccc5c5c(-c6cccc7c8ccccc8n(-c8ccccc8)c67)cccc54)cc4c3sc3ccccc34)c2)cc1 ; c1ccc(-n2c3ccccc3c3cccc(-c4cccc5c4c4ccccc4n5-c4cc(-c5cccc6c5sc5ccccc56)c5sc6ccccc6c5c4)c32)cc1]

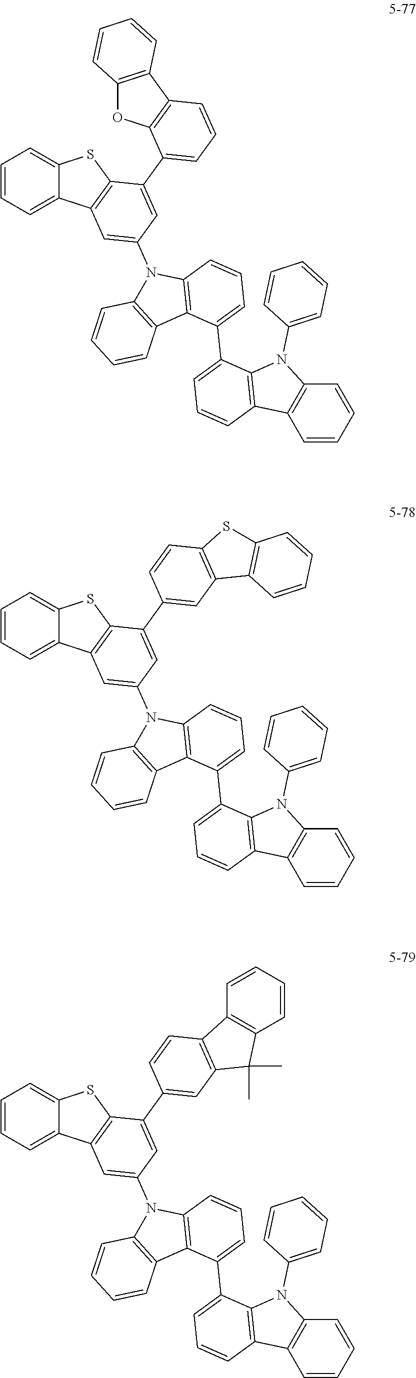 [SMILES: c1ccc(-n2c3ccccc3c3cccc(-c4cccc5c4c4ccccc4n5-c4cc(-c5cccc6c5oc5ccccc56)c5sc6ccccc6c5c4)c32)cc1 ; c1ccc(-n2c3ccccc3c3cccc(-c4cccc5c4c4ccccc4n5-c4cc(-c5ccc6sc7ccccc7c6c5)c5sc6ccccc6c5c4)c32)cc1 ; CC1(C)c2ccccc2-c2ccc(-c3cc(-n4c5ccccc5c5c(-c6cccc7c8ccccc8n(-c8ccccc8)c67)cccc54)cc4c3sc3ccccc34)cc21]

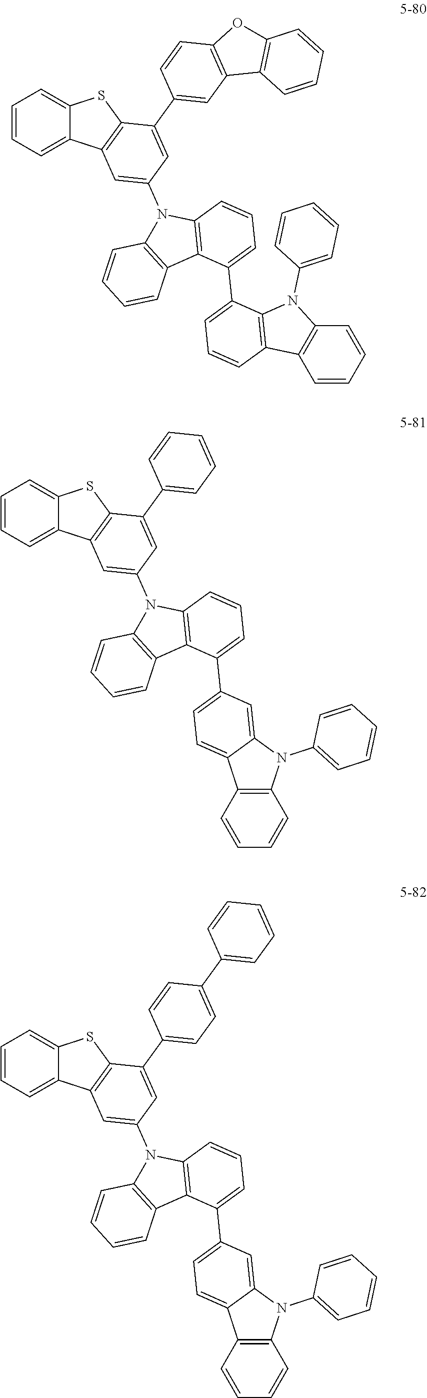 [SMILES: c1ccc(-n2c3ccccc3c3cccc(-c4cccc5c4c4ccccc4n5-c4cc(-c5ccc6oc7ccccc7c6c5)c5sc6ccccc6c5c4)c32)cc1 ; c1ccc(-c2cc(-n3c4ccccc4c4c(-c5ccc6c7ccccc7n(-c7ccccc7)c6c5)cccc43)cc3c2sc2ccccc23)cc1 ; c1ccc(-c2ccc(-c3cc(-n4c5ccccc5c5c(-c6ccc7c8ccccc8n(-c8ccccc8)c7c6)cccc54)cc4c3sc3ccccc34)cc2)cc1]

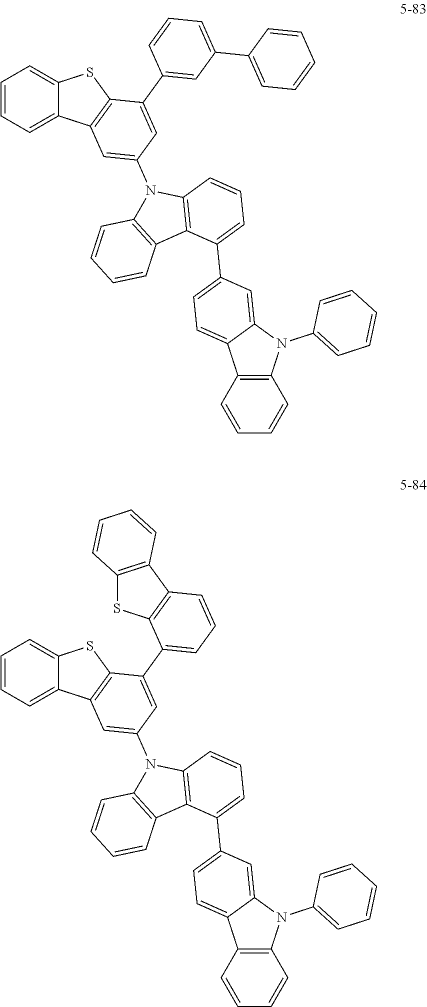 [SMILES: c1ccc(-c2cccc(-c3cc(-n4c5ccccc5c5c(-c6ccc7c8ccccc8n(-c8ccccc8)c7c6)cccc54)cc4c3sc3ccccc34)c2)cc1 ; c1ccc(-n2c3ccccc3c3ccc(-c4cccc5c4c4ccccc4n5-c4cc(-c5cccc6c5sc5ccccc56)c5sc6ccccc6c5c4)cc32)cc1]

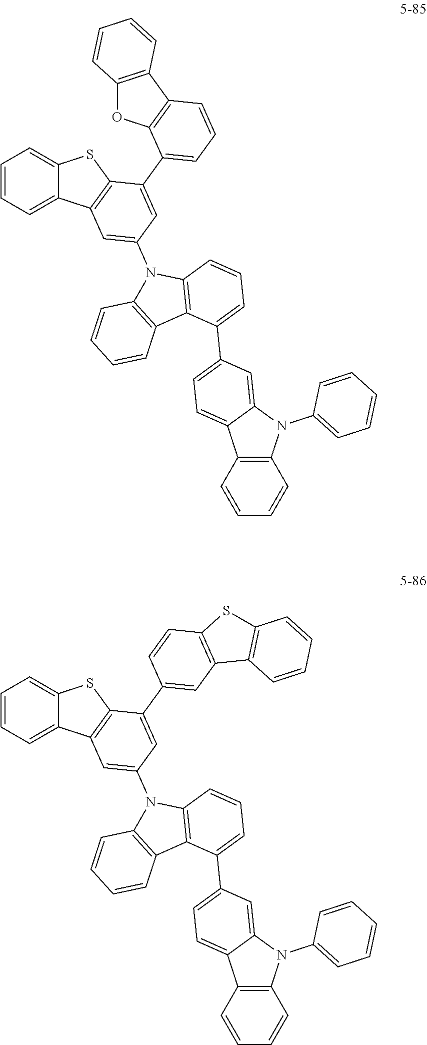 [SMILES: c1ccc(-n2c3ccccc3c3ccc(-c4cccc5c4c4ccccc4n5-c4cc(-c5cccc6c5oc5ccccc56)c5sc6ccccc6c5c4)cc32)cc1 ; c1ccc(-n2c3ccccc3c3ccc(-c4cccc5c4c4ccccc4n5-c4cc(-c5ccc6sc7ccccc7c6c5)c5sc6ccccc6c5c4)cc32)cc1]

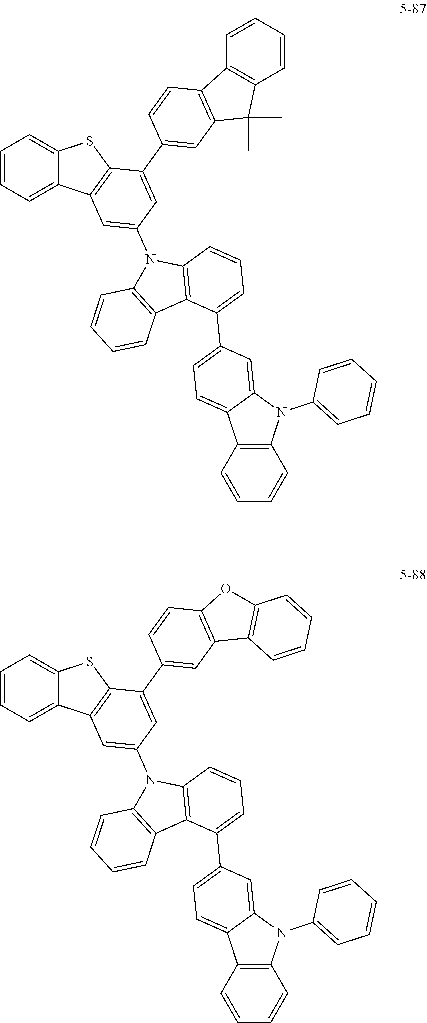 [SMILES: CC1(C)c2ccccc2-c2ccc(-c3cc(-n4c5ccccc5c5c(-c6ccc7c8ccccc8n(-c8ccccc8)c7c6)cccc54)cc4c3sc3ccccc34)cc21 ; c1ccc(-n2c3ccccc3c3ccc(-c4cccc5c4c4ccccc4n5-c4cc(-c5ccc6oc7ccccc7c6c5)c5sc6ccccc6c5c4)cc32)cc1]

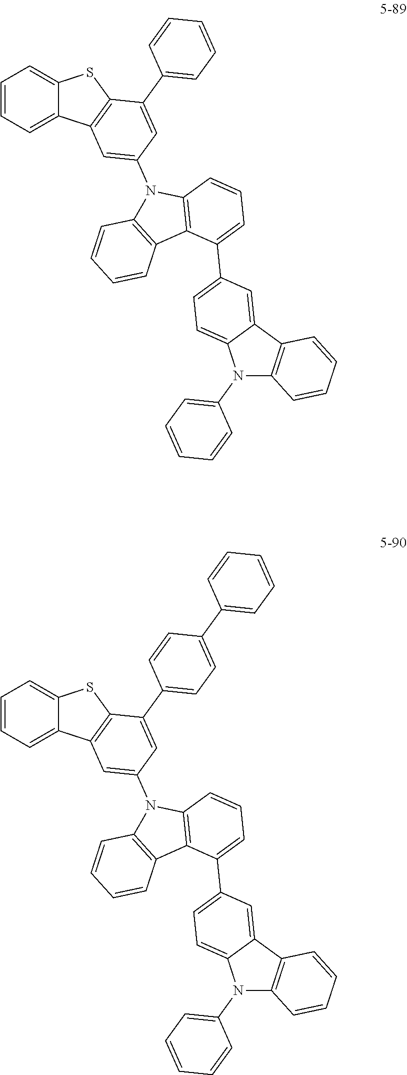 [SMILES: c1ccc(-c2cc(-n3c4ccccc4c4c(-c5ccc6c(c5)c5ccccc5n6-c5ccccc5)cccc43)cc3c2sc2ccccc23)cc1 ; c1ccc(-c2ccc(-c3cc(-n4c5ccccc5c5c(-c6ccc7c(c6)c6ccccc6n7-c6ccccc6)cccc54)cc4c3sc3ccccc34)cc2)cc1]

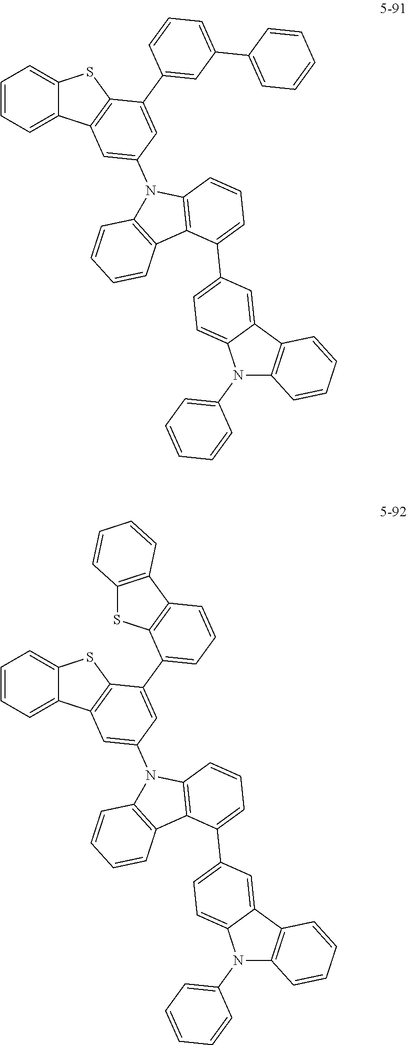 [SMILES: c1ccc(-c2cccc(-c3cc(-n4c5ccccc5c5c(-c6ccc7c(c6)c6ccccc6n7-c6ccccc6)cccc54)cc4c3sc3ccccc34)c2)cc1 ; c1ccc(-n2c3ccccc3c3cc(-c4cccc5c4c4ccccc4n5-c4cc(-c5cccc6c5sc5ccccc56)c5sc6ccccc6c5c4)ccc32)cc1]

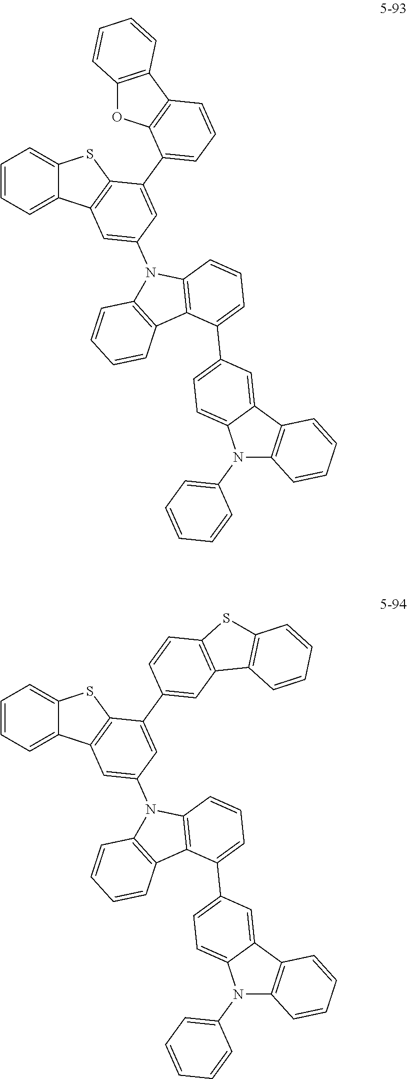 [SMILES: c1ccc(-n2c3ccccc3c3cc(-c4cccc5c4c4ccccc4n5-c4cc(-c5cccc6c5oc5ccccc56)c5sc6ccccc6c5c4)ccc32)cc1 ; c1ccc(-n2c3ccccc3c3cc(-c4cccc5c4c4ccccc4n5-c4cc(-c5ccc6sc7ccccc7c6c5)c5sc6ccccc6c5c4)ccc32)cc1]

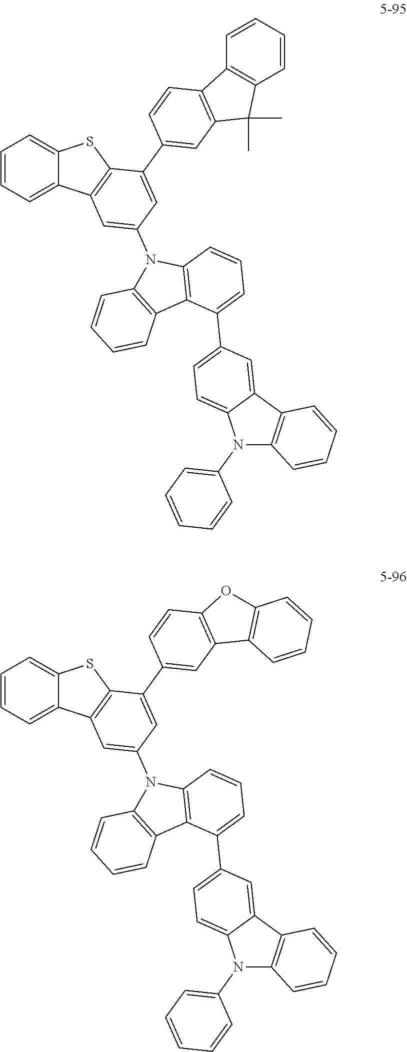 [SMILES: CC1(C)c2ccccc2-c2ccc(-c3cc(-n4c5ccccc5c5c(-c6ccc7c(c6)c6ccccc6n7-c6ccccc6)cccc54)cc4c3sc3ccccc34)cc21 ; c1ccc(-n2c3ccccc3c3cc(-c4cccc5c4c4ccccc4n5-c4cc(-c5ccc6oc7ccccc7c6c5)c5sc6ccccc6c5c4)ccc32)cc1]

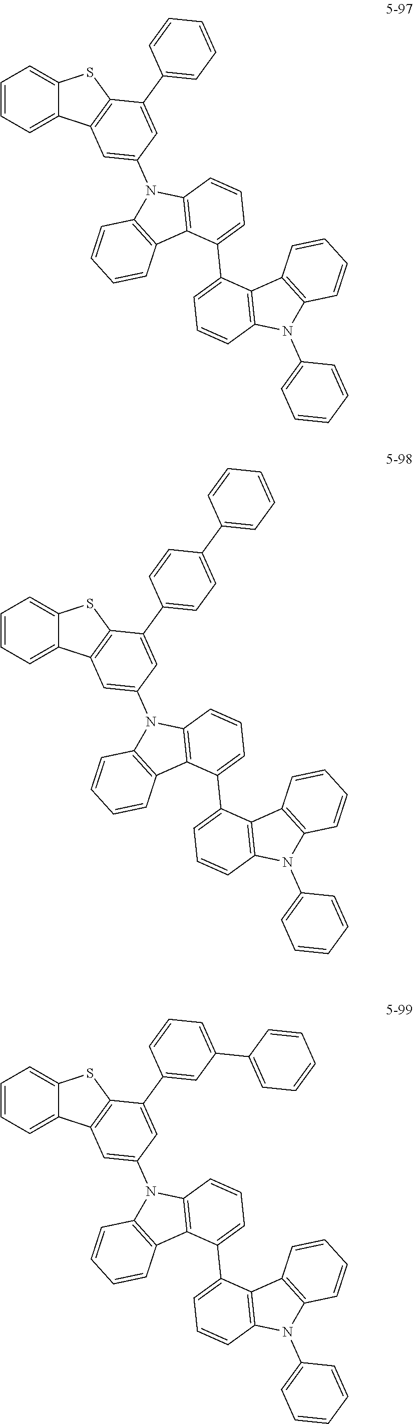 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-c3cc(-n4c5ccccc5c5c(-c6cccc7c6c6ccccc6n7-c6ccccc6)cccc54)cc4c3sc3ccccc34)cc2)cc1 ; c1ccc(-c2cccc(-c3cc(-n4c5ccccc5c5c(-c6cccc7c6c6ccccc6n7-c6ccccc6)cccc54)cc4c3sc3ccccc34)c2)cc1]

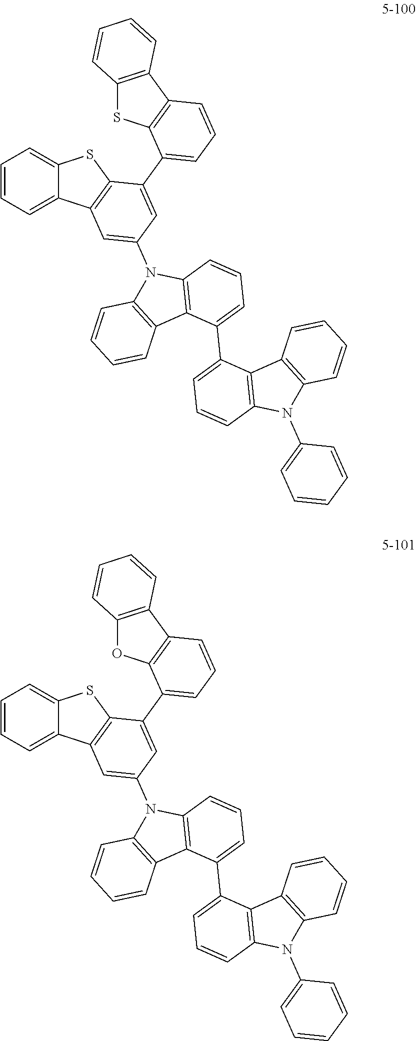 [SMILES: c1ccc(-n2c3ccccc3c3c(-c4cccc5c4c4ccccc4n5-c4cc(-c5cccc6c5sc5ccccc56)c5sc6ccccc6c5c4)cccc32)cc1 ; c1ccc(-n2c3ccccc3c3c(-c4cccc5c4c4ccccc4n5-c4cc(-c5cccc6c5oc5ccccc56)c5sc6ccccc6c5c4)cccc32)cc1]

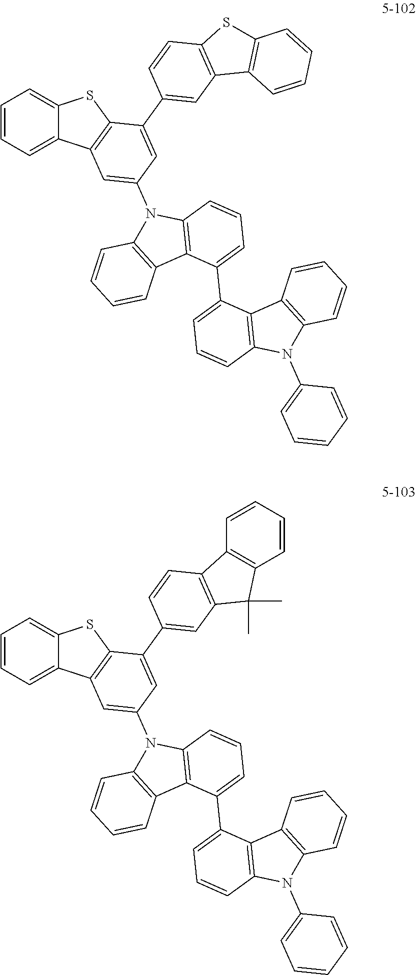 [SMILES: c1ccc(-n2c3ccccc3c3c(-c4cccc5c4c4ccccc4n5-c4cc(-c5ccc6sc7ccccc7c6c5)c5sc6ccccc6c5c4)cccc32)cc1 ; CC1(C)c2ccccc2-c2ccc(-c3cc(-n4c5ccccc5c5c(-c6cccc7c6c6ccccc6n7-c6ccccc6)cccc54)cc4c3sc3ccccc34)cc21]

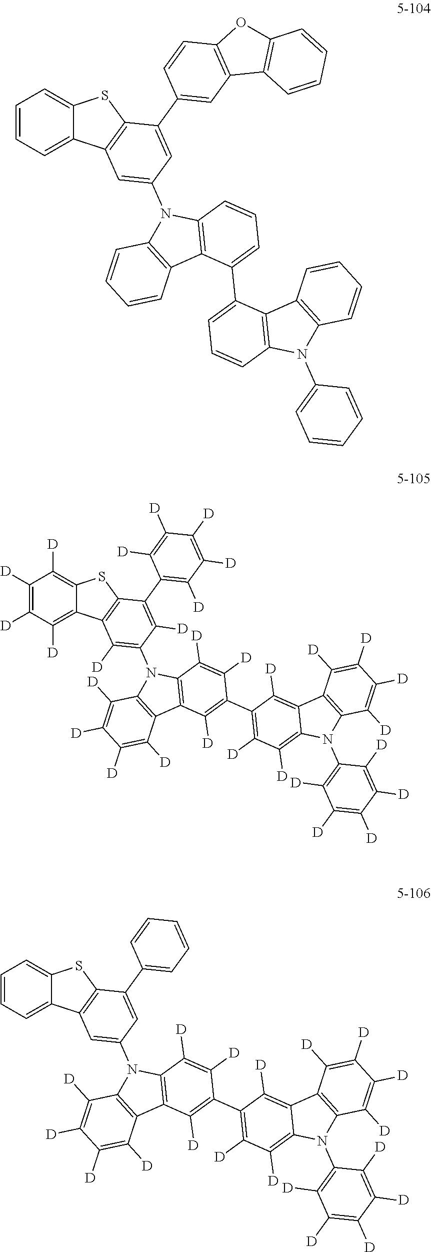

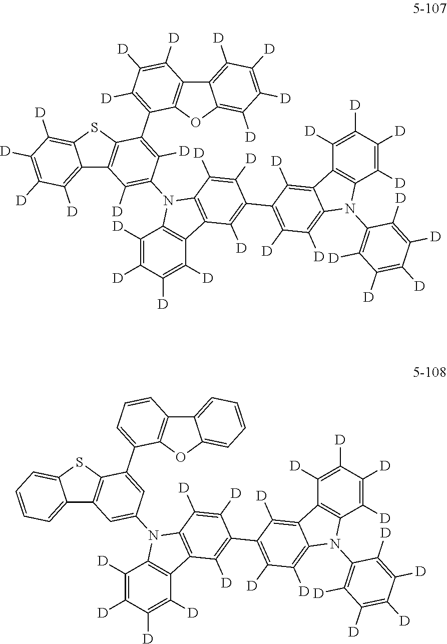 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c(-c5c([2H])c([2H])c([2H])c6c5oc5c([2H])c([2H])c([2H])c([2H])c56)c5sc6c([2H])c([2H])c([2H])c([2H])c6c5c4[2H])c([2H])c([2H])c32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5cccc6c5oc5ccccc56)c5sc6ccccc6c5c4)c([2H])c([2H])c32)c([2H])c1[2H]]

In one embodiment of the present application, Rd and Re are the same as or different from each other, and may be each independently hydrogen; deuterium; or a substituted or unsubstituted aryl group having 6 to 60 carbon atoms.

In one embodiment of the present application, Rd and Re are the same as or different from each other, and may be each independently hydrogen; deuterium; or a substituted or unsubstituted aryl group having 6 to 40 carbon atoms.

In one embodiment of the present application, Rd and Re are the same as or different from each other, and may be each independently hydrogen; deuterium; or a substituted or unsubstituted aryl group having 6 to 20 carbon atoms.

In one embodiment of the present application, Rd and Re are the same as or different from each other, and may be each independently hydrogen; deuterium; a substituted or unsubstituted phenyl group; or a substituted or unsubstituted biphenyl group.

In one embodiment of the present application, Rd and Re are the same as or different from each other, and may be each independently hydrogen; deuterium; a phenyl group; or a biphenyl group.

In one embodiment of the present application, Rd and Re are the same as or different from each other, and may be each independently hydrogen; or deuterium.

In one embodiment of the present application, Rd and Re are hydrogen.

In one embodiment of the present application, Rd and Re are deuterium.

In another embodiment, r is an integer of 0 to 7, and when r is 2 or greater, Rds are the same as or different from each other, and two or more Rds adjacent to each other may bond to each other to form a substituted or unsubstituted aliphatic or aromatic hydrocarbon ring or heteroring.

In one embodiment of the present application, R32 and R33 are the same as or different from each other, and each independently a substituted or unsubstituted silyl group; a substituted or unsubstituted aryl group having 6 to 60 carbon atoms; or a substituted or unsubstituted heteroaryl group having 2 to 60 carbon atoms.

In one embodiment of the present application, R32 and R33 are the same as or different from each other, and each independently a substituted or unsubstituted silyl group; a substituted or unsubstituted aryl group having 6 to 40 carbon atoms; or a substituted or unsubstituted heteroaryl group having 2 to 40 carbon atoms.

In one embodiment of the present application, R32 and R33 are the same as or different from each other, and may be each independently a substituted or unsubstituted silyl group; a substituted or unsubstituted phenyl group; a substituted or unsubstituted biphenyl group; a substituted or unsubstituted terphenyl group; a substituted or unsubstituted triphenyl group; a substituted or unsubstituted naphthyl group; a substituted or unsubstituted dibenzothiophene group; or a substituted or unsubstituted dibenzofuran group.

According to one embodiment of the present application, Chemical Formula 14 may be represented by any one of the following compounds, but is not limited thereto.

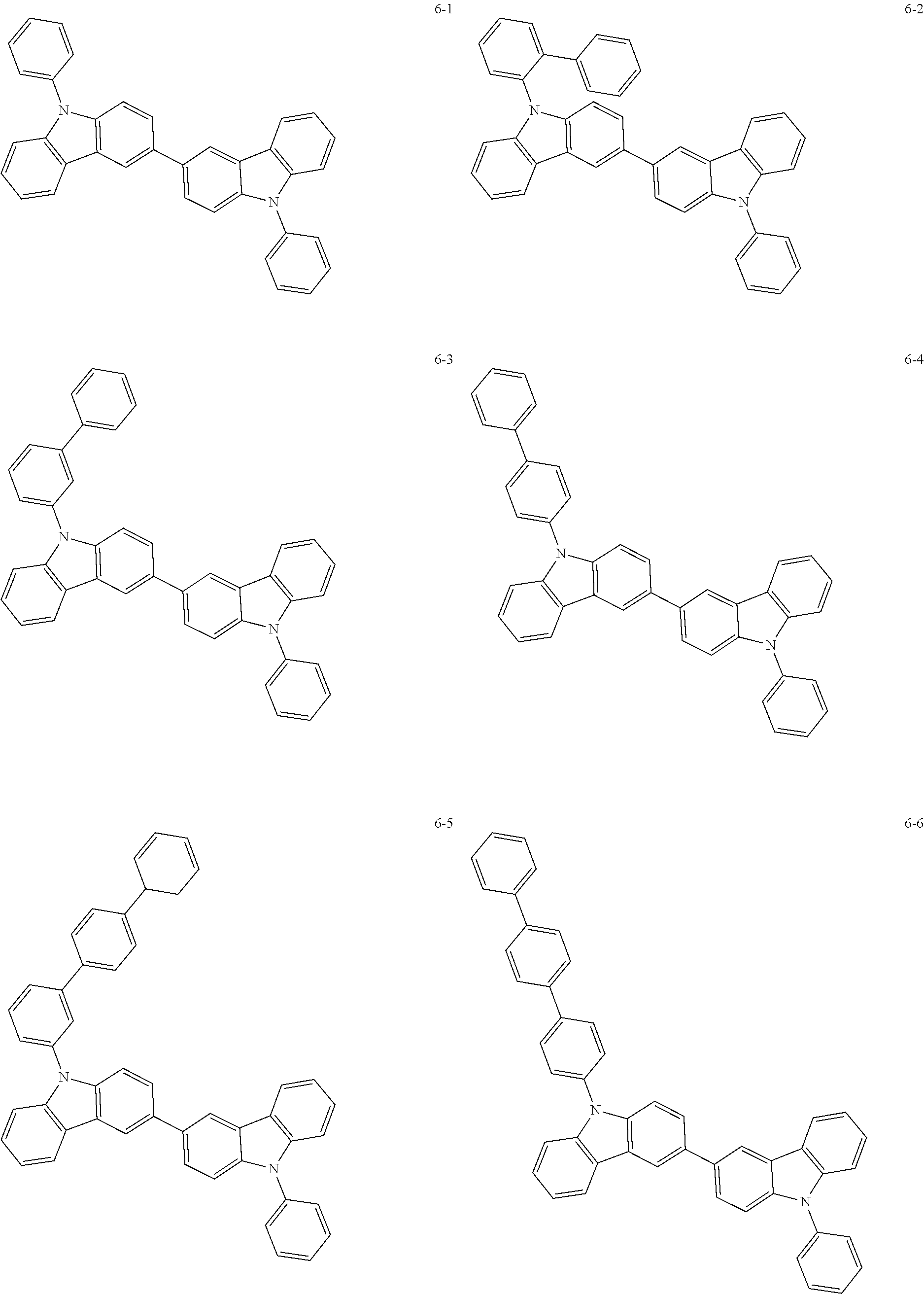 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; C1=CCC(c2ccc(-c3cccc(-n4c5ccccc5c5cc(-c6ccc7c(c6)c6ccccc6n7-c6ccccc6)ccc54)c3)cc2)C=C1 ; c1ccc(-c2ccc(-c3ccc(-n4c5ccccc5c5cc(-c6ccc7c(c6)c6ccccc6n7-c6ccccc6)ccc54)cc3)cc2)cc1]

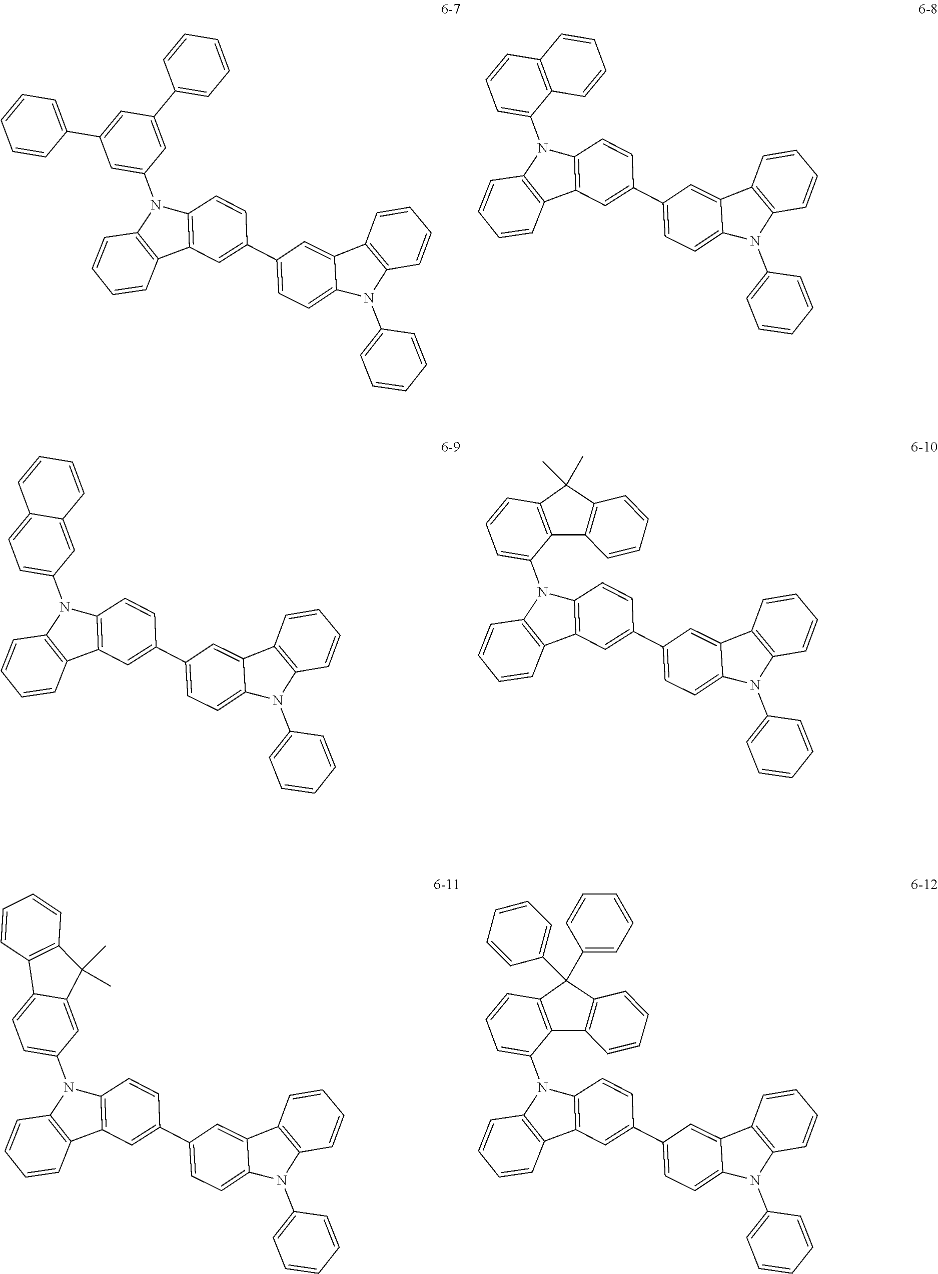 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

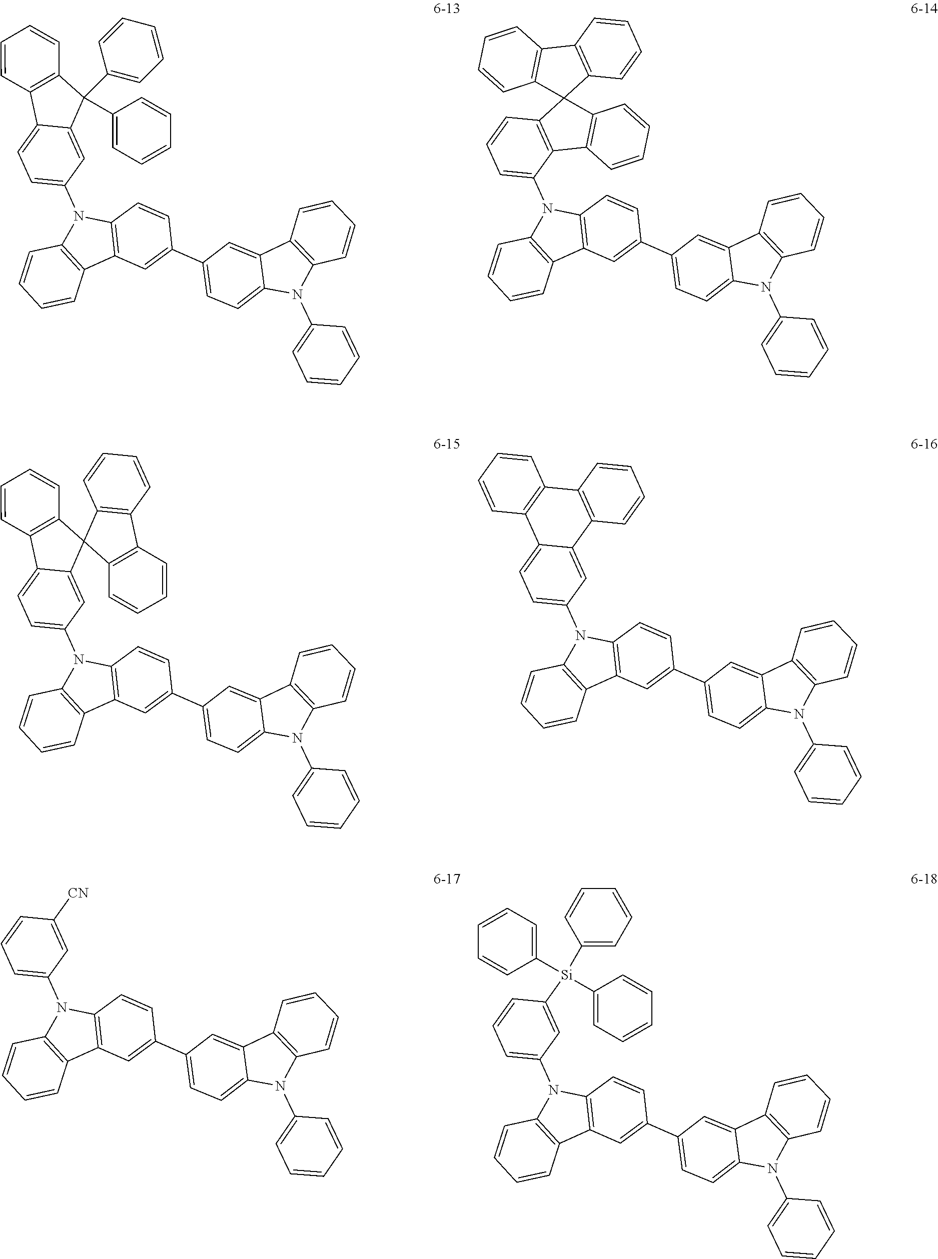

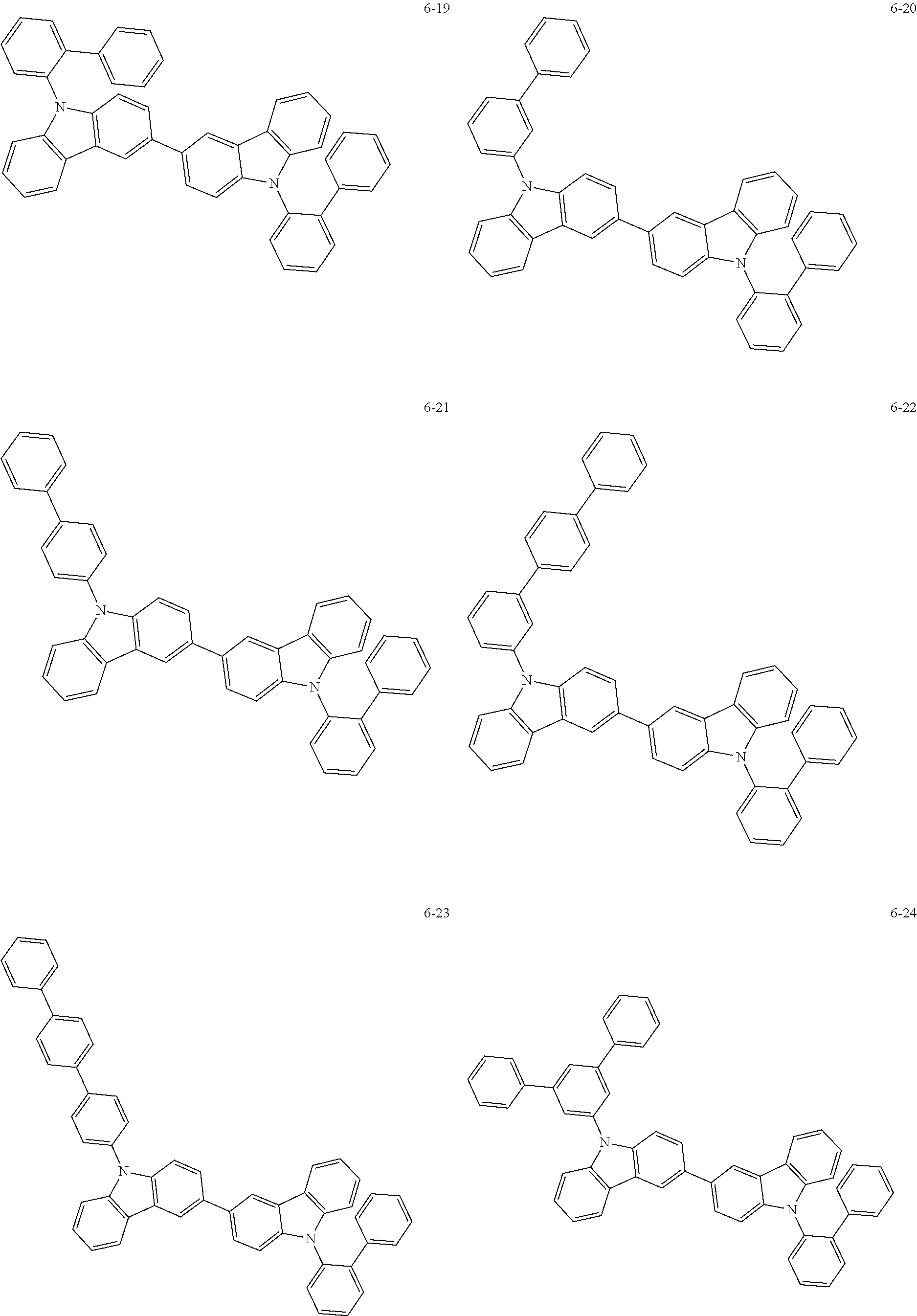

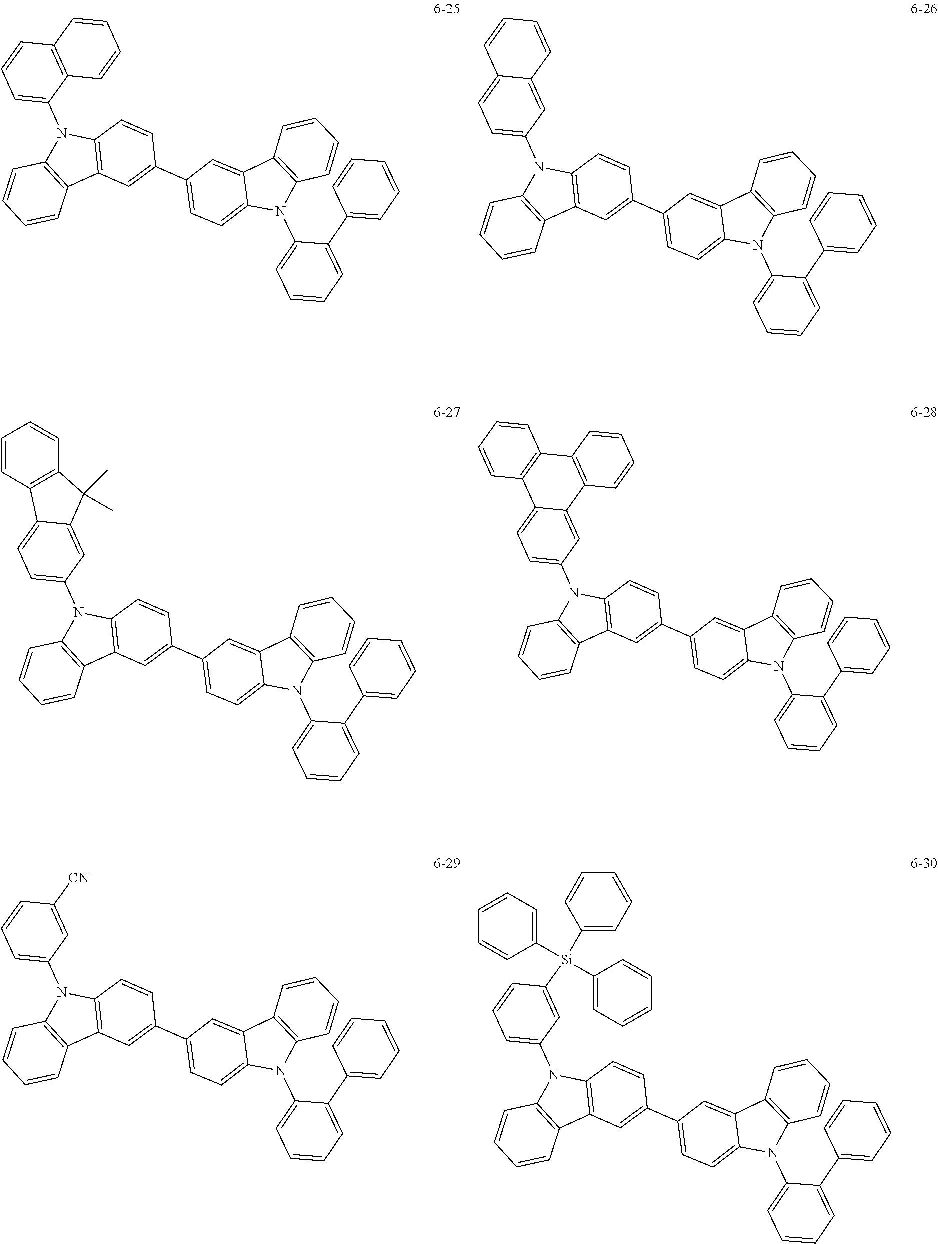

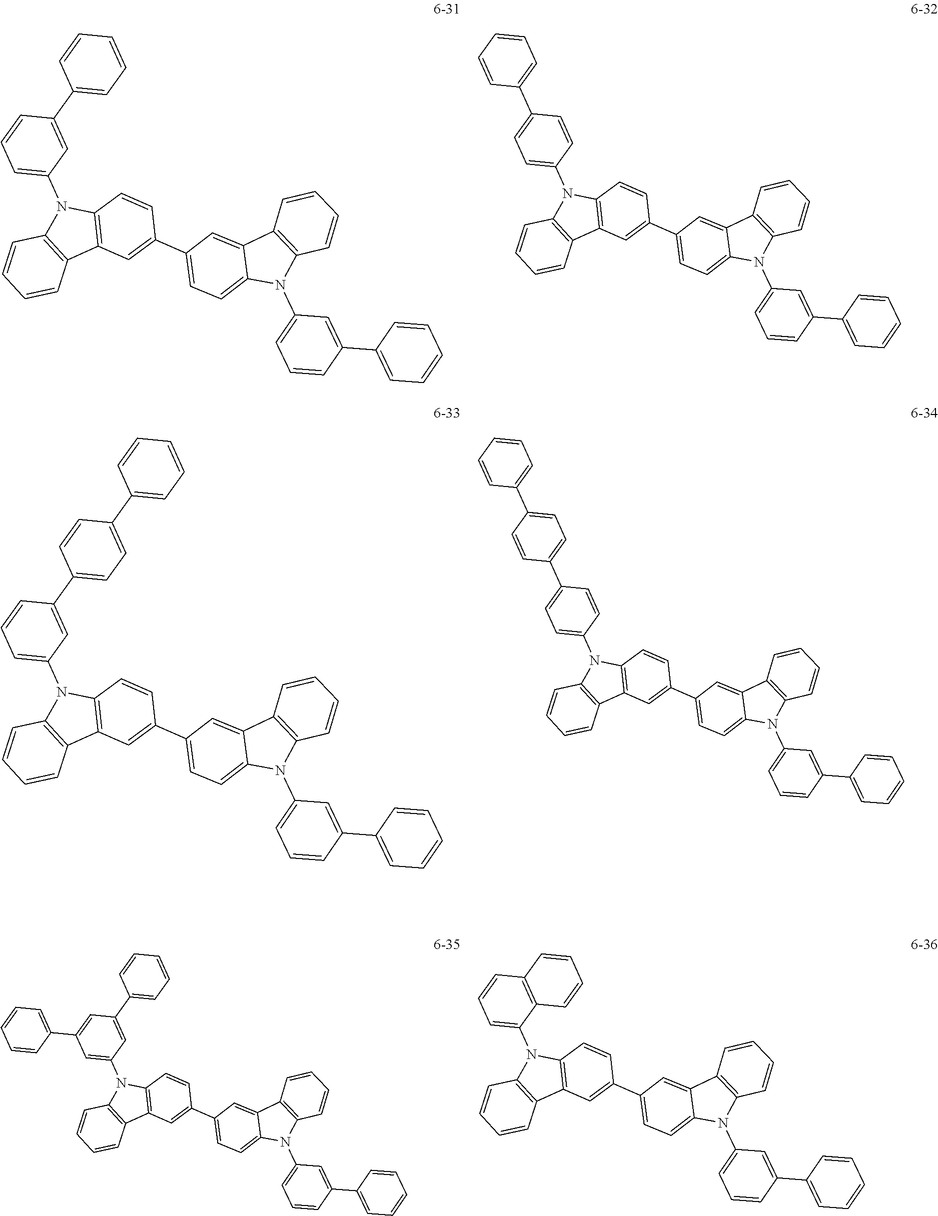

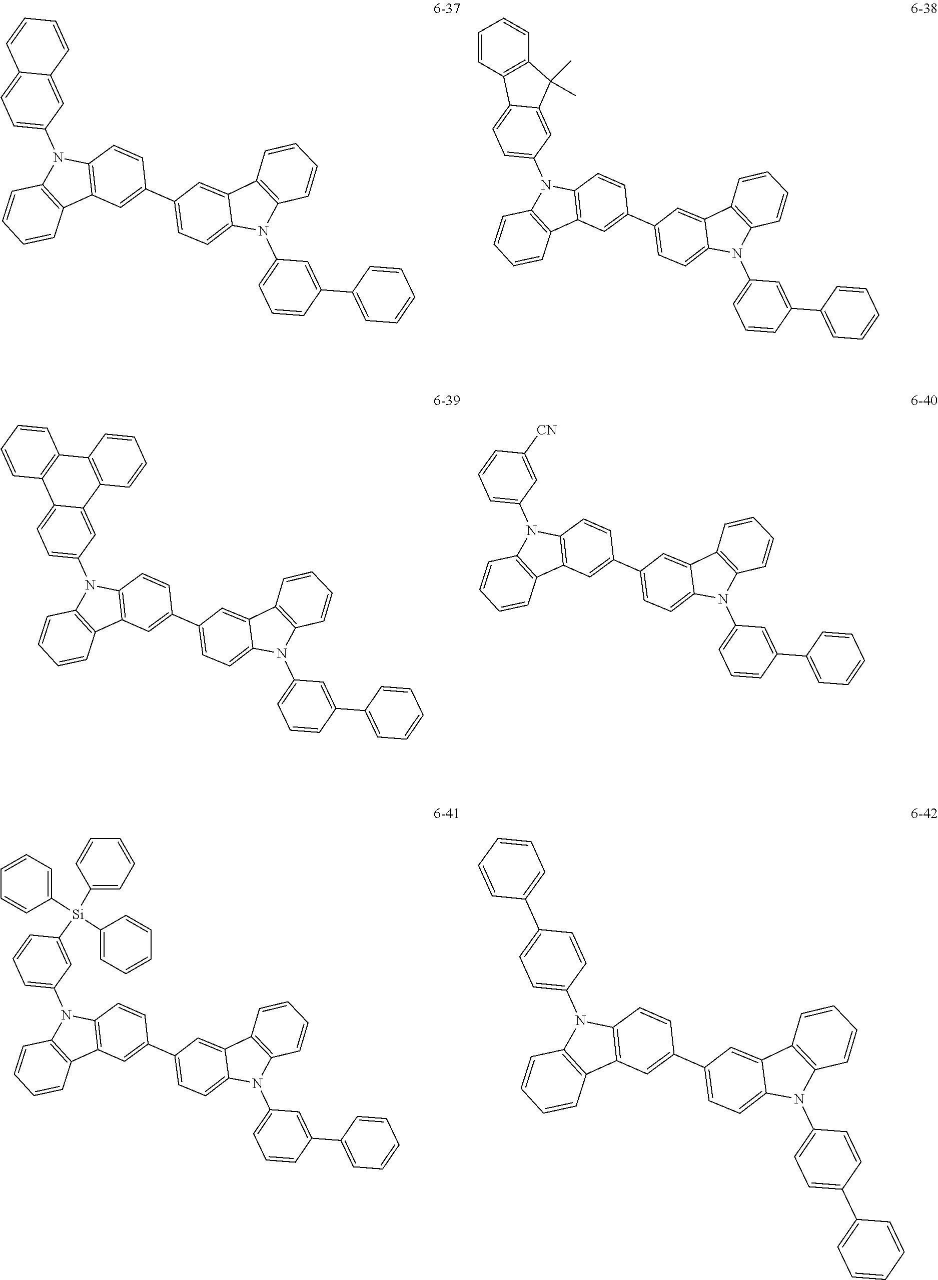

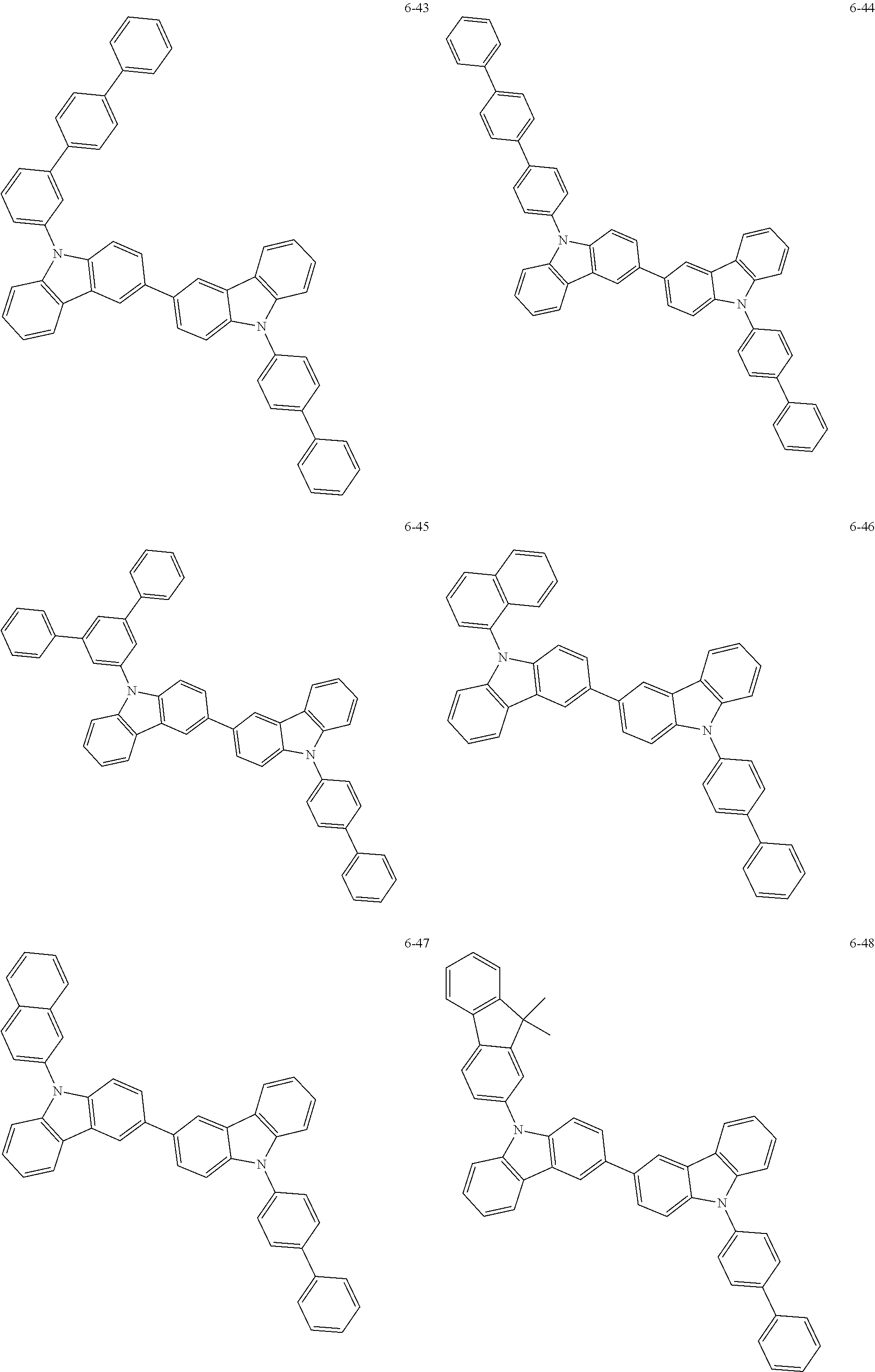

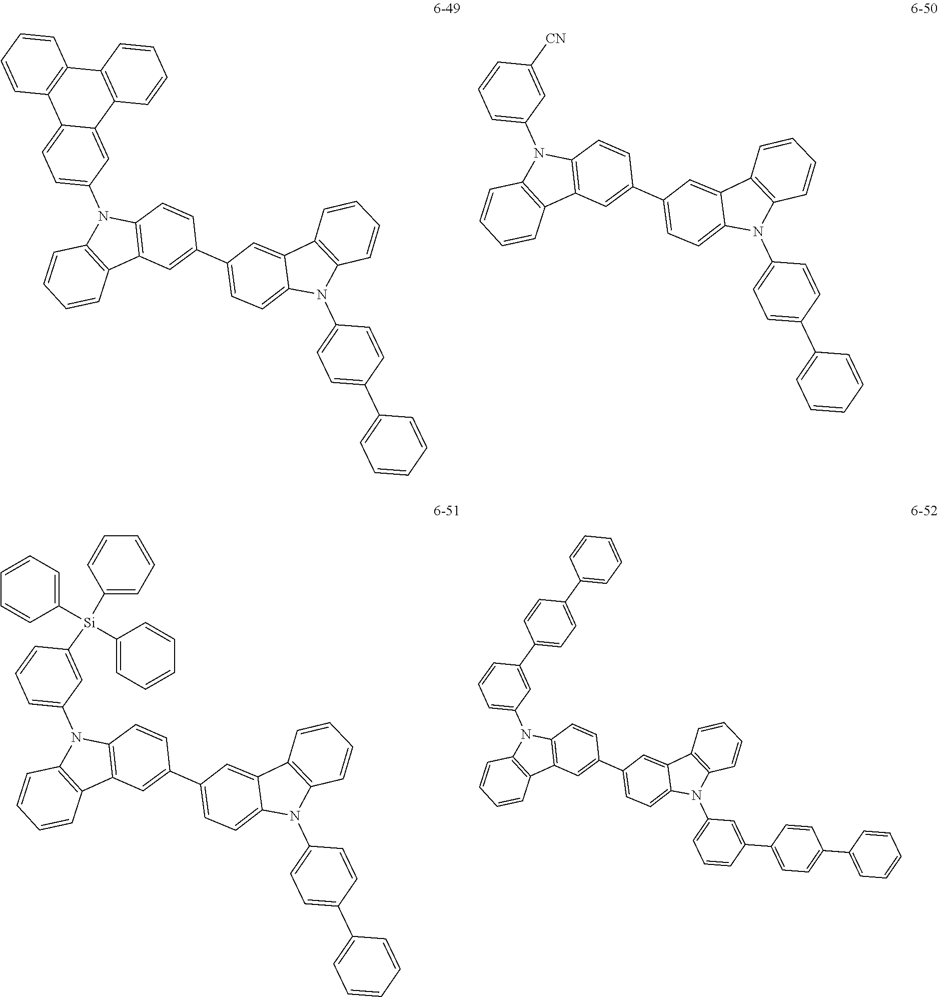 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

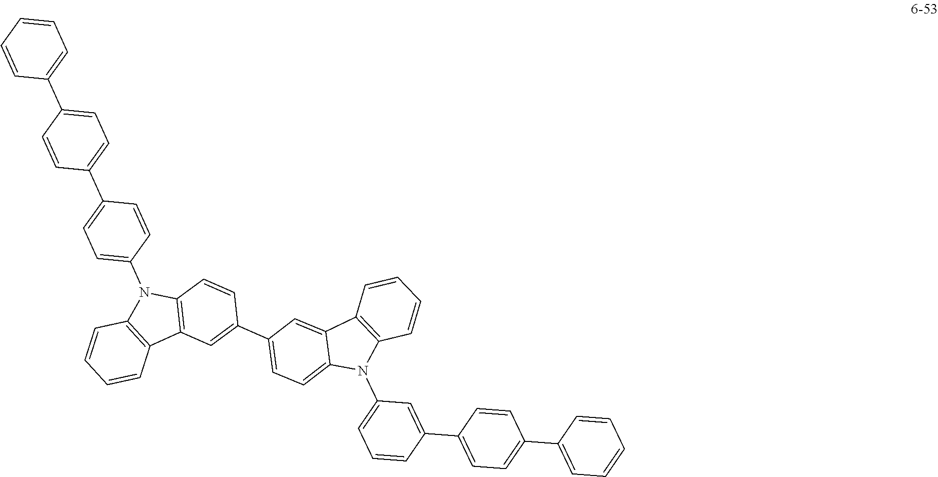 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1]

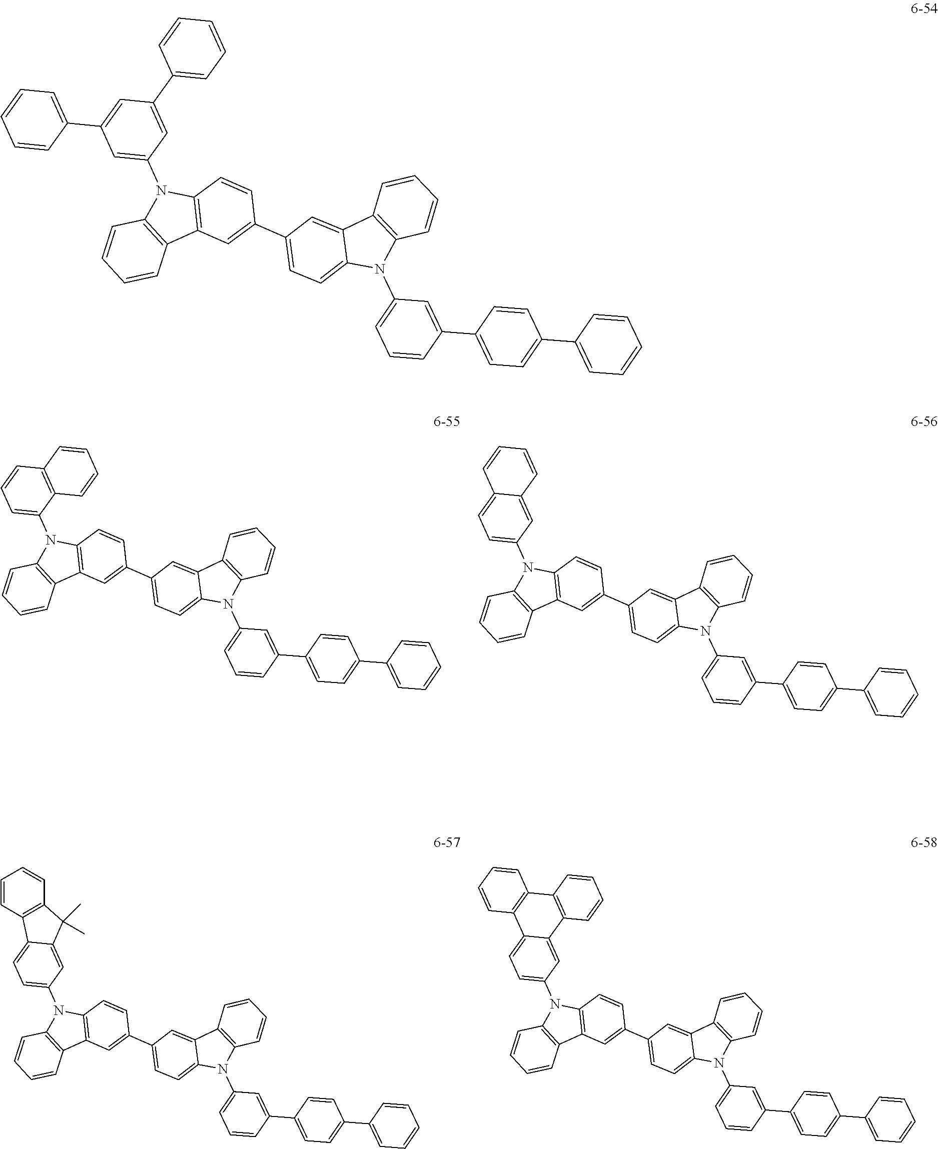

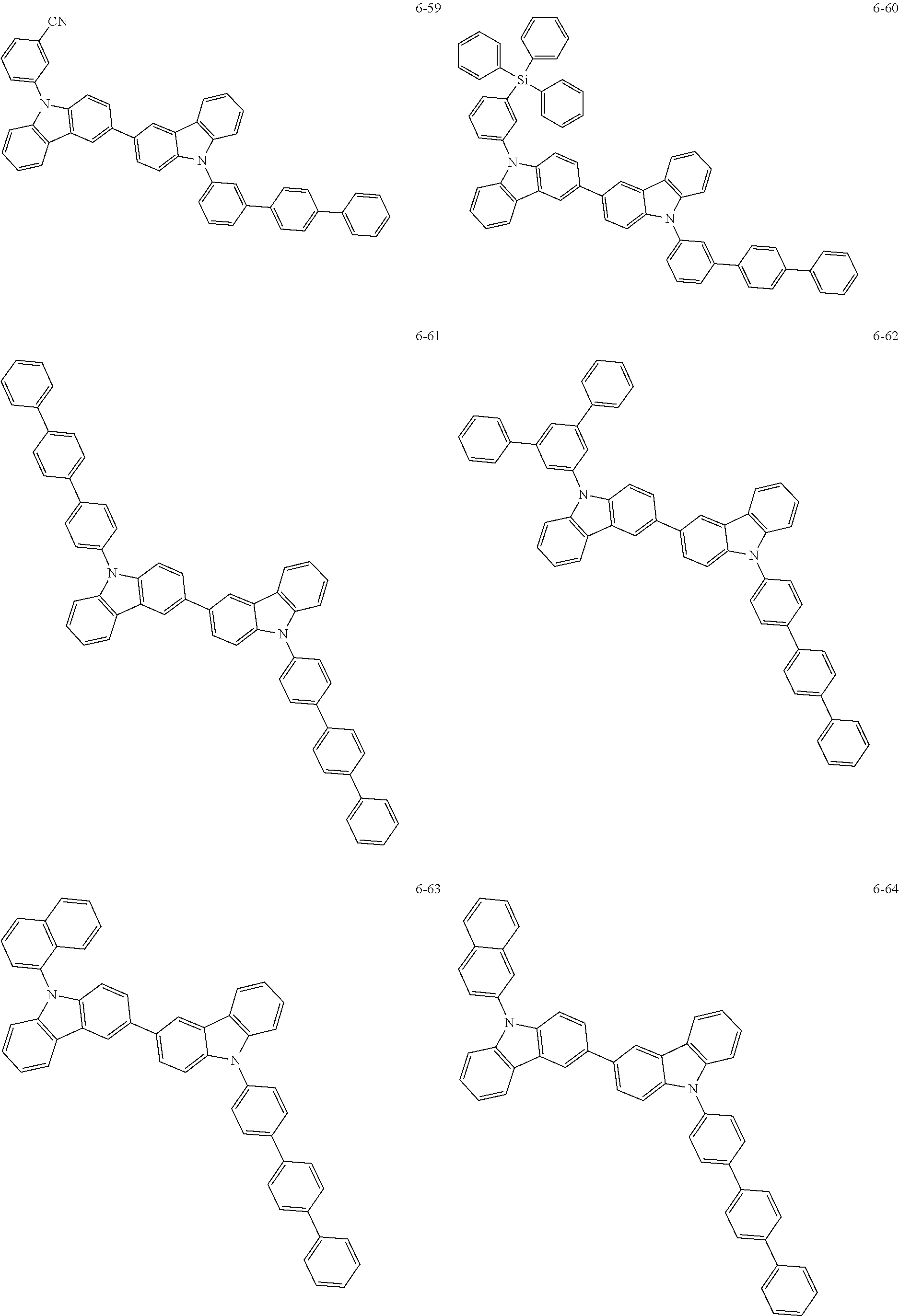

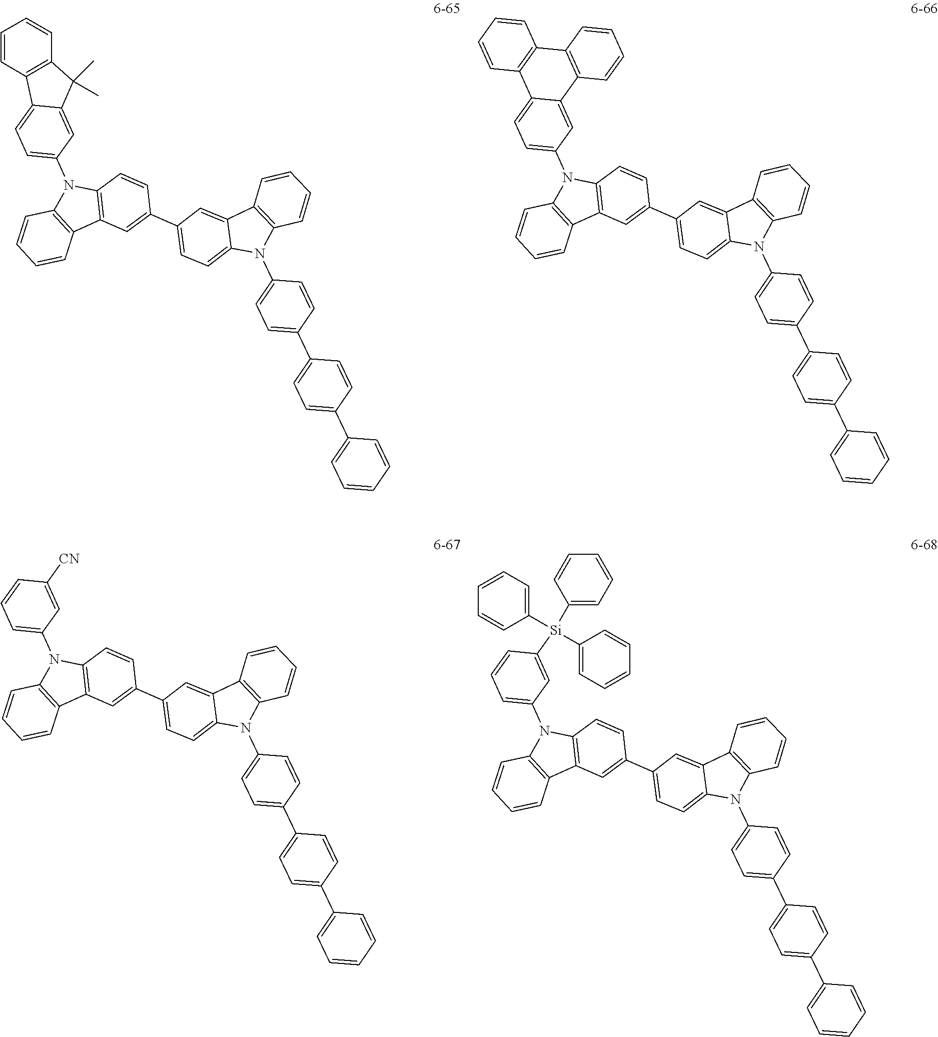 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

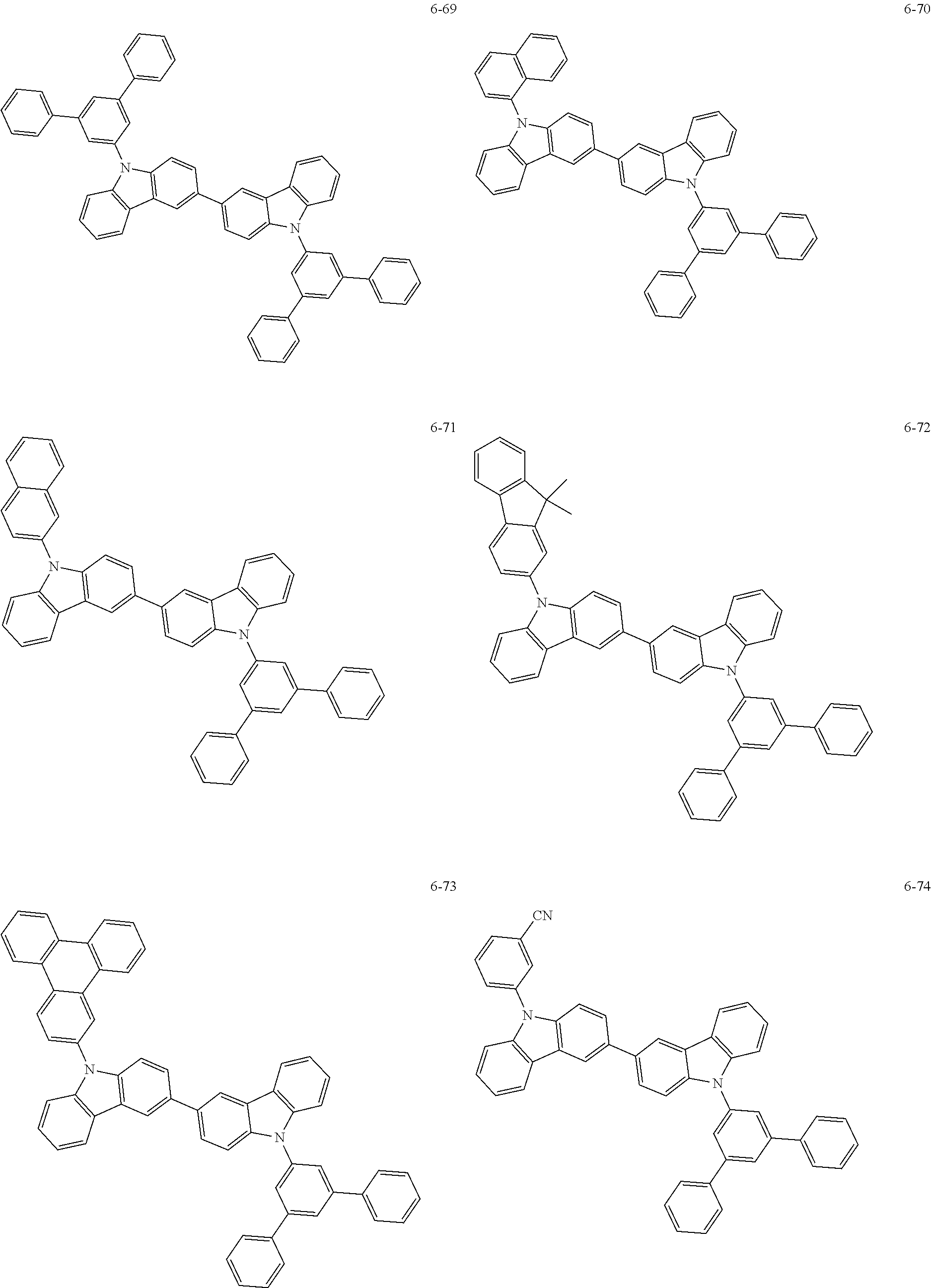

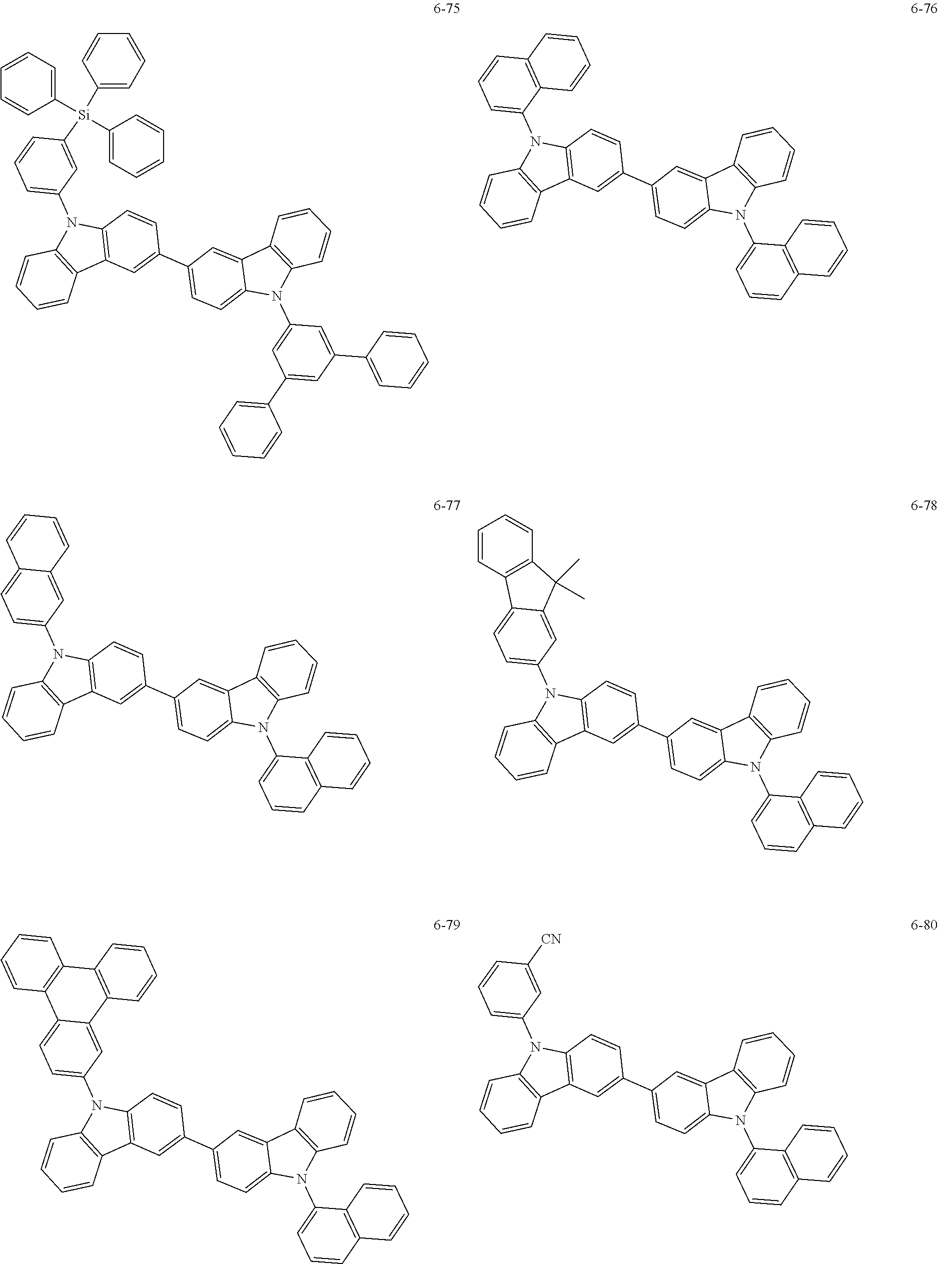

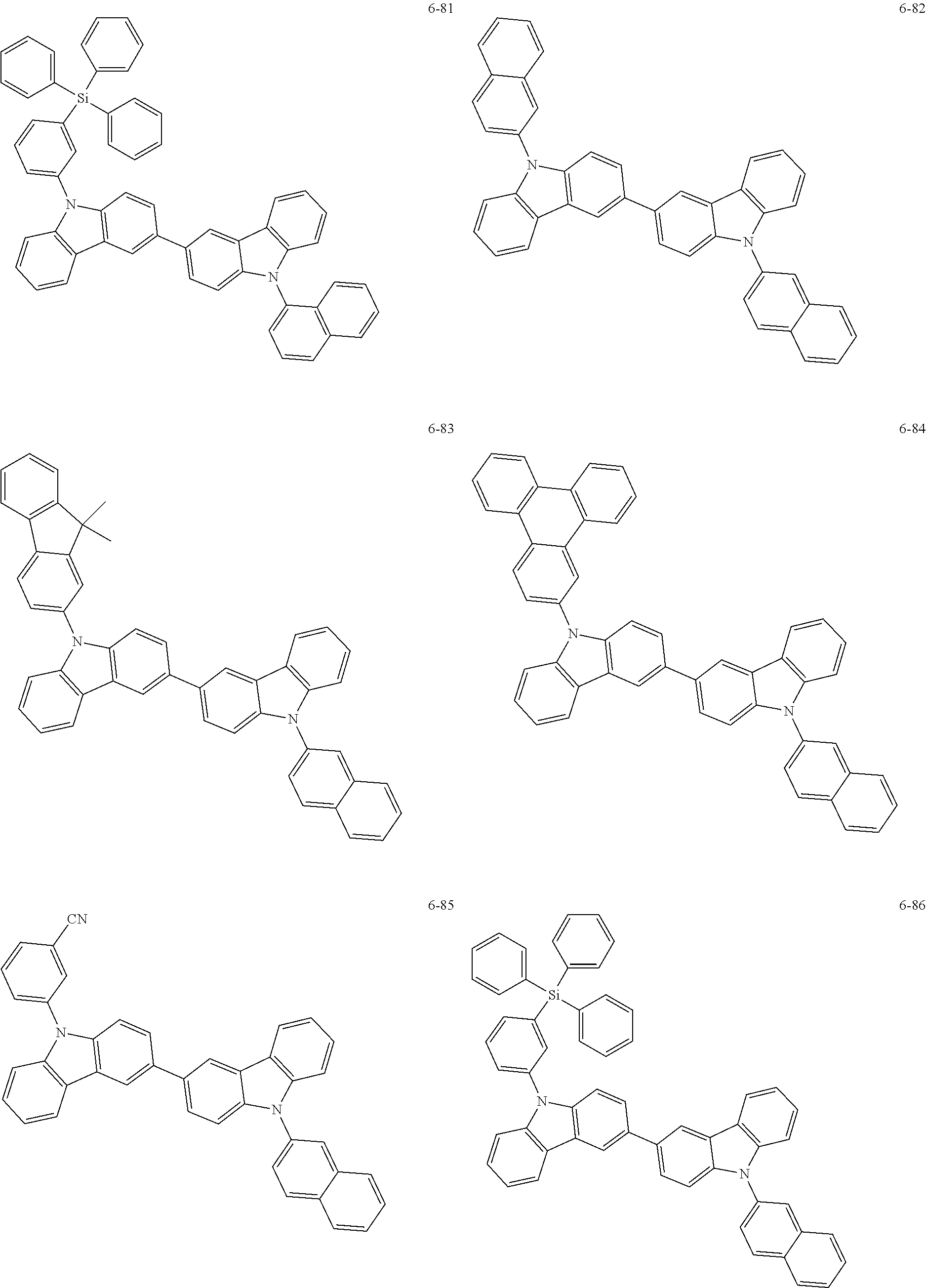

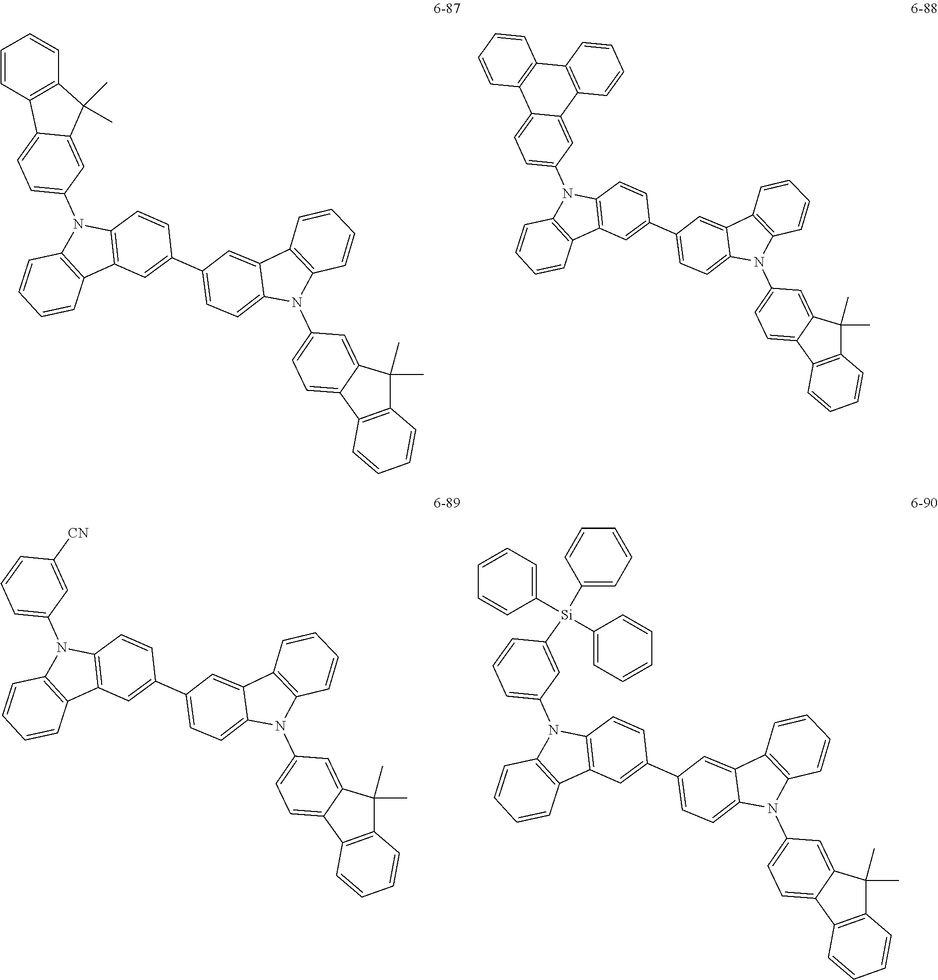 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

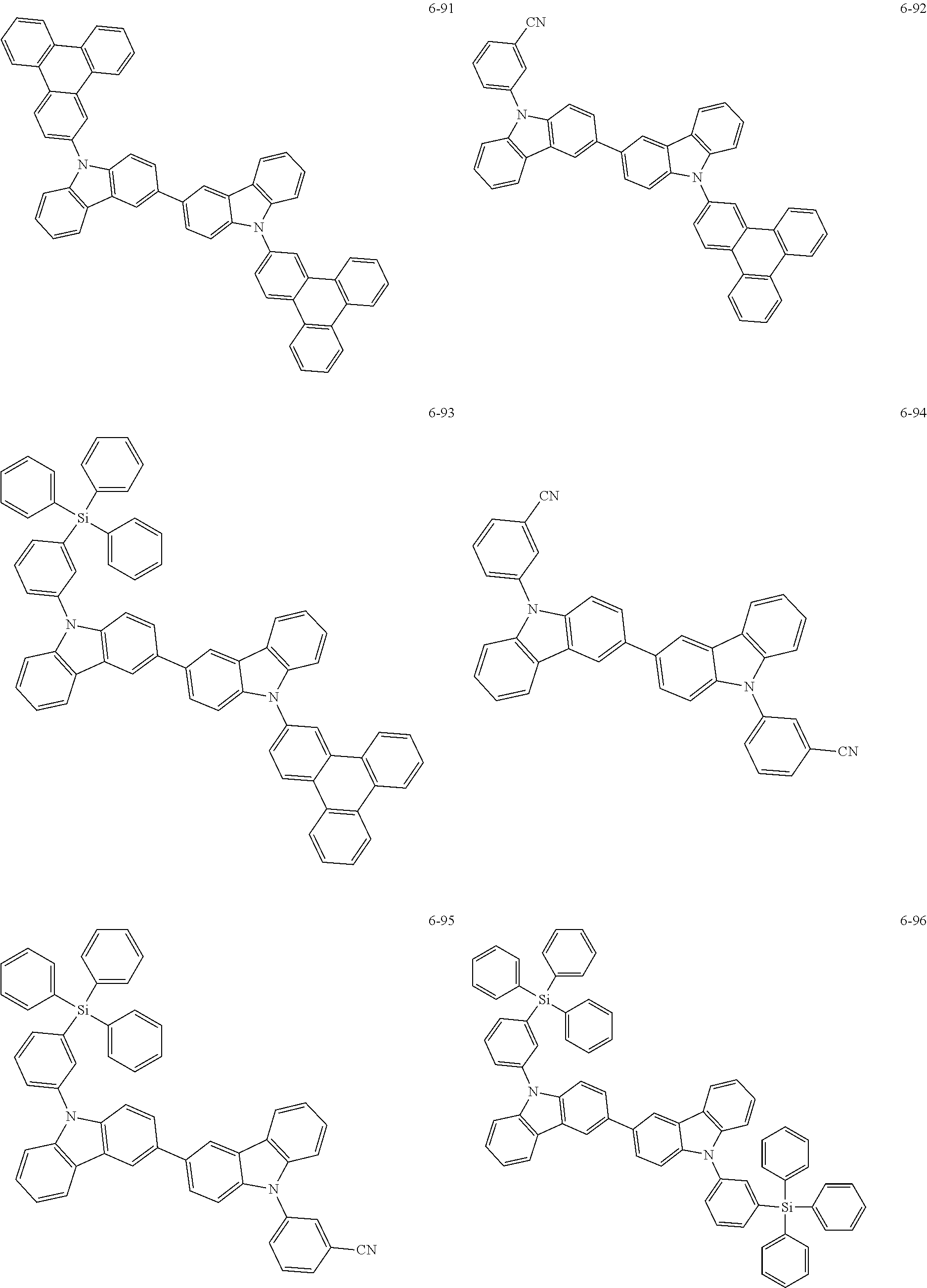

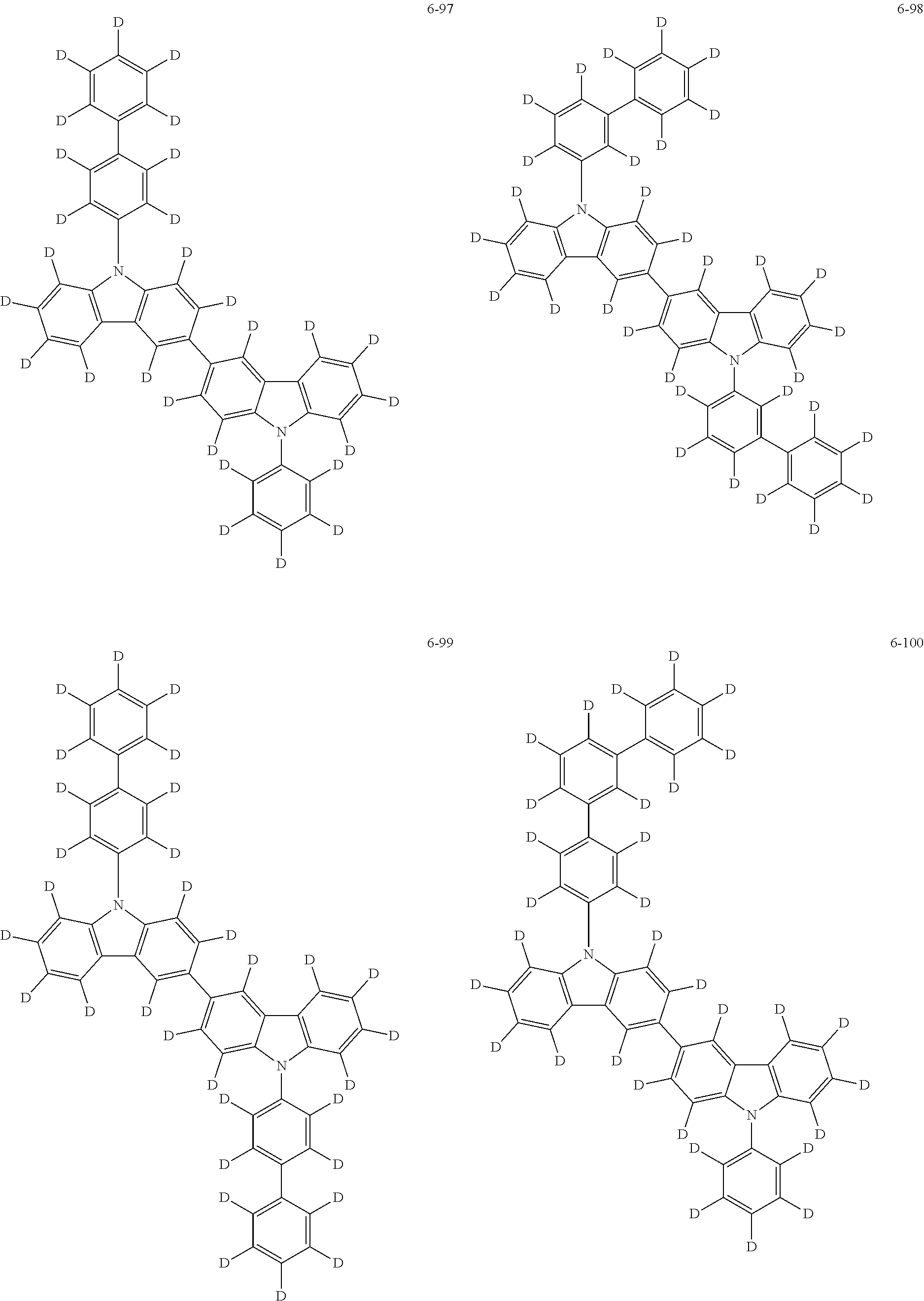

In addition, the organic material layer comprises one or more light emitting layers, and the light emitting layer comprises the heterocyclic compound represented by Chemical Formula 1 as a first compound, and further comprises the heterocyclic compound represented by Chemical Formula 13 or 14 as a second compound. When comprising the heterocyclic compound represented by Chemical Formula 1 in the light emitting layer among the organic material layers, the organic light emitting device has more superior light emission efficiency and lifetime.

In addition, by introducing various substituents to the structure of Chemical Formula 13 or 14, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 13 or 14, the energy band gap may be finely controlled, and meanwhile, properties at interfaces between organic materials are enhanced, and material applications may become diverse.

Meanwhile, the heterocyclic compound has a high glass transition temperature (Tg), and has excellent thermal stability. Such an increase in the thermal stability becomes an important factor providing driving stability to a device.

The heterocyclic compound according to one embodiment of the present application may be prepared using a multi-step chemical reaction. Some intermediate compounds are prepared first, and from the intermediate compounds, the heterocyclic compound of Chemical Formula 13 or 14 may be prepared. More specifically, the heterocyclic compound according to one embodiment of the present application may be prepared based on preparation examples to describe later.

In addition, the organic material layer comprises one or more light emitting layers, and the light emitting layer further comprises one of the heterocyclic compound represented by Chemical Formula 1 and the heterocyclic compound represented by Chemical Formula 13. When comprising one of the heterocyclic compound represented by Chemical Formula 1 and the heterocyclic compound represented by Chemical Formula 13 in the light emitting layer among the organic material layers, the organic light emitting device has more superior light emission efficiency and lifetime due to an exciplex phenomenon.

In addition, the organic material layer comprises one or more light emitting layers, and the light emitting layer further comprises one of the heterocyclic compound represented by Chemical Formula 1 and the heterocyclic compound represented by Chemical Formula 14. When comprising one of the heterocyclic compound represented by Chemical Formula 1 and the heterocyclic compound represented by Chemical Formula 14 in the light emitting layer among the organic material layers, the organic light emitting device has more superior light emission efficiency and lifetime due to an exciplex phenomenon.

In the organic light emitting device of the present application, the organic material layer comprises a light emitting layer, and the light emitting layer may comprise the heterocyclic compound as a host material of a light emitting material.

In the organic light emitting device of the present application, the light emitting layer may comprise two or more host materials, and at least one of the host materials may comprise the heterocyclic compound as a host material of a light emitting material.

In the organic light emitting device of the present application, two or more host materials may be pre-mixed and used as the light emitting layer, and at least one of the two or more host materials may comprise the heterocyclic compound as a host material of a light emitting material.

The pre-mixing means placing and mixing two or more host materials of the light emitting layer in one source of supply before depositing on the organic material layer.

In the organic light emitting device of the present application, the light emitting layer may comprise two or more host materials, and the two or more host materials may each comprise one or more p-type host materials and n-type host materials, and at least one of the host materials may comprise the heterocyclic compound as a host material of a light emitting material. In this case, the organic light emitting device may have superior driving, efficiency and lifetime.

The organic light emitting device of the present disclosure may further comprise one, two or more layers selected from the group consisting of a light emitting layer, a hole injection layer, a hole transfer layer, an electron injection layer, an electron transfer layer, a hole auxiliary layer and a hole blocking layer.

The organic light emitting device according to one embodiment of the present application may be manufactured using common organic light emitting device manufacturing methods and materials except that the organic material layer is formed using the heterocyclic compound described above.

In addition, another embodiment of the present application provides a composition for an organic material layer of an organic light emitting device, the composition comprising the heterocyclic compound represented by Chemical Formula 1 and the heterocyclic compound represented by Chemical Formula 13 or 14.

In another embodiment of the present application, the heterocyclic compound represented by Chemical Formula 1:the heterocyclic compound represented by Chemical Formula 13 or 14 may have a weight ratio of 1:10 to 10:1, 1:8 to 8:1, 1:5 to 5:1 or 1:2 to 2:1 in the composition, however, the weight ratio is not limited thereto.

Specific descriptions on the heterocyclic compound represented by Chemical Formula 1 and the compound represented by Chemical Formula 13 or 14 are the same as the descriptions provided above.

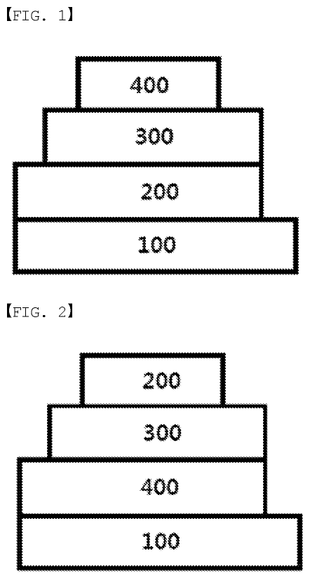

FIG. 1 to FIG. 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

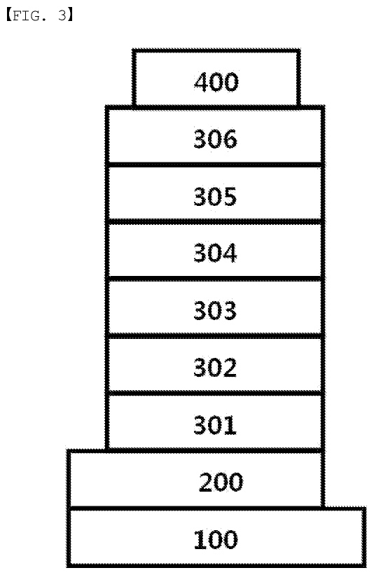

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 includes a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

In the organic light emitting device according to one embodiment of the present application, materials other than the heterocyclic compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

The organic light emitting device according to one embodiment of the present application may be a top-emission type, a bottom-emission type or a dual-emission type depending on the materials used.

The heterocyclic compound according to one embodiment of the present application may also be used in an organic electronic device including an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### PREPARATION EXAMPLE

### <Preparation Example 1> Preparation of Target Compound 1-86

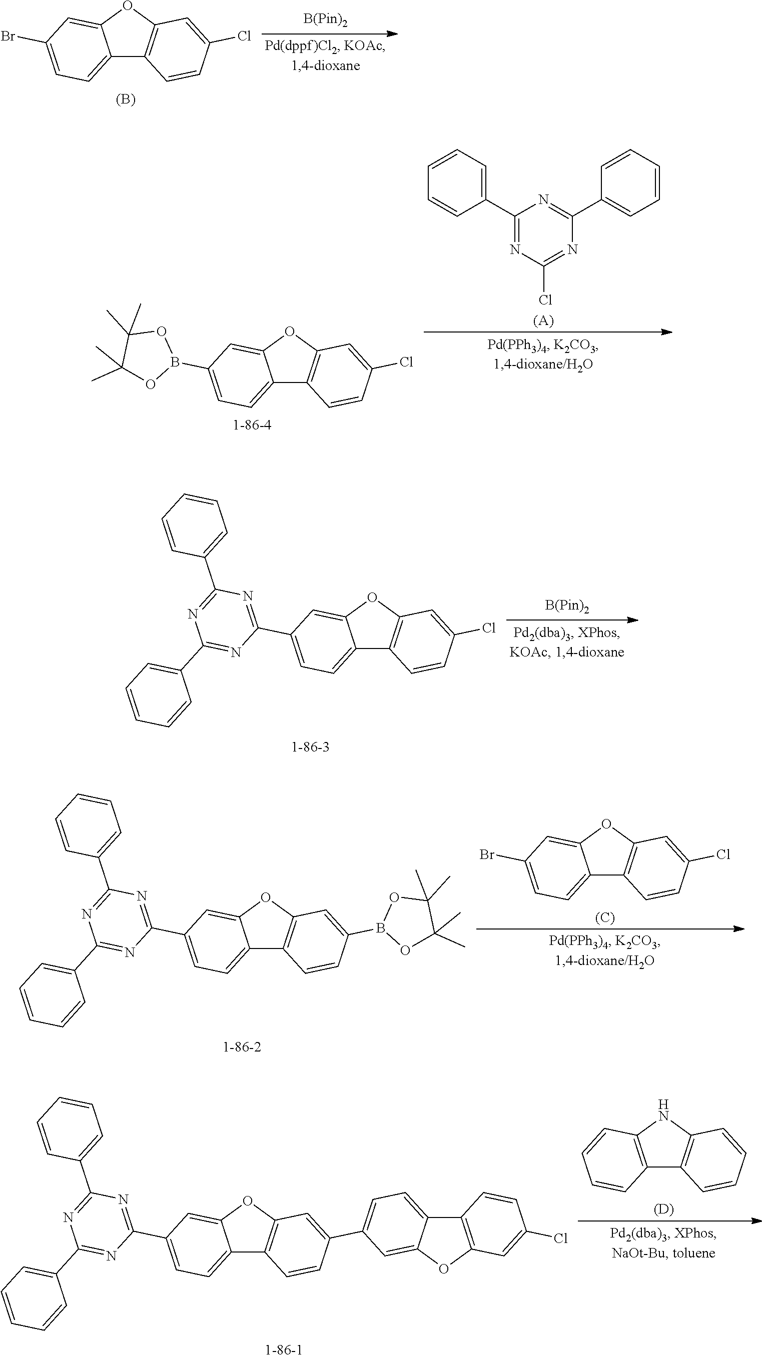

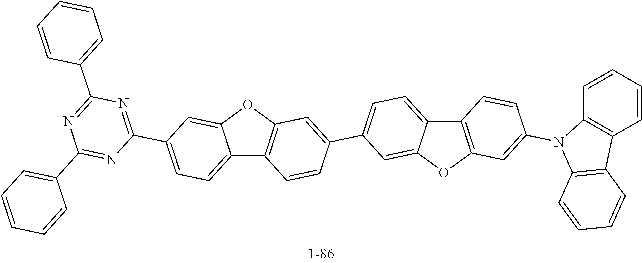 [SMILES: c1ccc(-c2nc(-c3ccccc3)nc(-c3ccc4c(c3)oc3cc(-c5ccc6c(c5)oc5cc(-n7c8ccccc8c8ccccc87)ccc56)ccc34)n2)cc1]

1) Preparation of Compound 1-86-4

3-Bromo-7-chlorodibenzo[b,d]furan (B) (16.8 g, 59.8 mM), B(Pin)₂ (22.8 g, 89.7 mM), Pd(dppf)Cl₃ (2.2 g, 3.0 mM) and KOAc (17.6 g, 179.4 mM) were dissolved in 1,4-dioxane (300 mL), and refluxed for 12 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex=1:2) to obtain Compound 1-86-4 (35.8 g, 82%). The Hex means hexane.

### 2) Preparation of Compound 1-86-3

Compound 1-86-4 (35.8 g, 49.0 mM), 2-chloro-4,6-diphenyl-1,3,5-triazine (A) (15.7 g, 58.8 mM), Pd(PPh₃)₄ (2.8 g, 2.4 mM), and K₂CO₃ (13.5 g, 98.0 mM) were dissolved in 1,4-dioxane/H₂O (600 mL/120 mL), and refluxed for 12 hours. After the reaction was completed, the result was extracted by introducing distilled water and dichloromethane (DCM) thereto at room temperature, and after drying the organic layer with MgSO₄, the solvent was removed using a rotary evaporator. The reaction material was recrystallized with methanol to obtain Compound 1-86-3 (18.2 g, 86%).

### 3) Preparation of Compound 1-86-2

Compound 1-86-3 (18.2 g, 41.9 mM), B(Pin)₂ (16.0 g, 62.8 mM), Pd₂(dba)₃ (1.9 g, 2.1 mM), XPhos (2.0 g, 4.2 mM) and KOAc (12.3 g, 125.7 mM) were dissolved in 1,4-dioxane (300 mL), and refluxed for 12 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex=1:2) to obtain Compound 1-86-2 (17.8 g, 81%).

### 4) Preparation of Compound 1-86-1

Compound 1-86-2 (17.8 g, 33.9 mM), 3-bromo-7-chlorodibenzo[b,d]furan (C) (11.4 g, 40.7 mM), Pd(PPh₃)₄ (2.0 g, 1.7 mM) and K₂CO₃ (9.3 g, 67.8 mM) were dissolved in 1,4-dioxane/H₂O (300 mL/60 mL), and refluxed for 12 hours. After the reaction was completed, the result was extracted by introducing distilled water and dichloromethane (DCM) thereto at room temperature, and after drying the organic layer with MgSO₄, the solvent was removed using a rotary evaporator. The reaction material was recrystallized with methanol to obtain Compound 1-86-1 (17.7 g, 87%).

### 5) Preparation of Target Compound 1-86

Compound 1-86-1 (17.7 g, 29.5 mM), 9H-carbazole (D) (5.9 g, 35.4 mM), Pd₂(dba)₃ (1.4 g, 1.5 mM), XPhos (1.4 g, 3.0 mM) and NaOt-Bu (5.7 g, 59.0 mM) were dissolved in 1,4-dioxane (300 mL), and refluxed for 12 hours. After the reaction was completed, the result was extracted by introducing distilled water and dichloromethane (DCM) thereto at room temperature, and after drying the organic layer with MgSO₄, the solvent was removed using a rotary evaporator. The reaction material was recrystallized with methanol to obtain target Compound 1-86 (19.2 g, 89%).

Target Compounds of the following Table 1 were synthesized in the same manner as in Preparation Example 1 except that Intermediate A of the following Table 1 was used instead of 2-chloro-4,6-diphenyl-1,3,5-triazine in Preparation Example 1-2), Intermediate B was used instead of 3-bromo-7-chlorodibenzo[b,d]furan in Preparation Example 1-1), Intermediate C was used instead of 3-bromo-7-chlorodibenzo[b,d]furan in Preparation Example 1-4), and Intermediate D was used instead of 9H-carbazole in Preparation Example 1-5).

TABLE 1

| Compound | | | | | |
|---|---|---|---|---|---|
| No. | Intermediate A | Intermediate B | Intermediate C | Intermediate D | Target Compound |
| 1-1 | 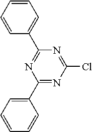 [SMILES: Clc1nc(-c2ccccc2)nc(-c2ccccc2)n1] | 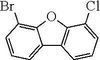 [SMILES: Clc1cccc2c1oc1c(Br)cccc12] | | 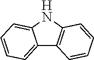 [SMILES: c1ccc2c(c1)[nH]c1ccccc12] | 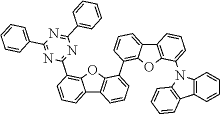 [SMILES: c1ccc(-c2nc(-c3ccccc3)nc(-c3cccc4c3oc3c(-c5cccc6c5oc5c(-n7c8ccccc8c8ccccc87)cccc56)cccc34)n2)cc1] |
| 1-87 | | 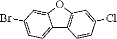 [SMILES: Clc1ccc2c(c1)oc1cc(Br)ccc12] | 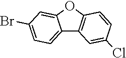 [SMILES: Clc1ccc2oc3cc(Br)ccc3c2c1] | | 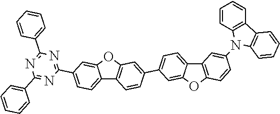 [SMILES: c1ccc(-c2nc(-c3ccccc3)nc(-c3ccc4c(c3)oc3cc(-c5ccc6c(c5)oc5ccc(-n7c8ccccc8c8ccccc87)cc56)ccc34)n2)cc1] |
| 1-94 | | | 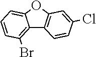 [SMILES: Clc1ccc2c(c1)oc1cccc(Br)c12] | | 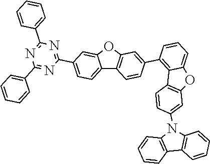 [SMILES: c1ccc(-c2nc(-c3ccccc3)nc(-c3ccc4c(c3)oc3cc(-c5cccc6oc7cc(-n8c9ccccc9c9ccccc98)ccc7c56)ccc34)n2)cc1] |
| 1-95 | | | 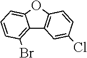 [SMILES: Clc1ccc2oc3cccc(Br)c3c2c1] | | 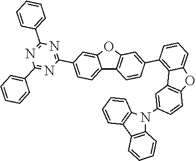 [SMILES: c1ccc(-c2nc(-c3ccccc3)nc(-c3ccc4c(c3)oc3cc(-c5cccc6oc7ccc(-n8c9ccccc9c9ccccc98)cc7c56)ccc34)n2)cc1] |
| 1-102 | | 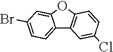 [SMILES: Clc1ccc2oc3cc(Br)ccc3c2c1] | 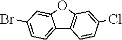 [SMILES: Clc1ccc2c(c1)oc1cc(Br)ccc12] | | 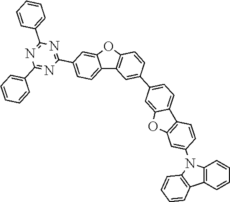 [SMILES: c1ccc(-c2nc(-c3ccccc3)nc(-c3ccc4c(c3)oc3ccc(-c5ccc6c(c5)oc5cc(-n7c8ccccc8c8ccccc87)ccc56)cc34)n2)cc1] |
| 1-103 | | | 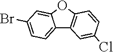 [SMILES: Clc1ccc2oc3cc(Br)ccc3c2c1] | | 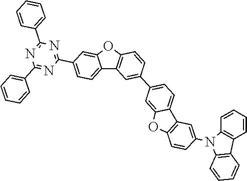 [SMILES: c1ccc(-c2nc(-c3ccccc3)nc(-c3ccc4c(c3)oc3ccc(-c5ccc6c(c5)oc5ccc(-n7c8ccccc8c8ccccc87)cc56)cc34)n2)cc1] |
| 1-110 | | | 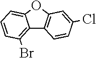 [SMILES: Clc1ccc2c(c1)oc1cccc(Br)c12] | | 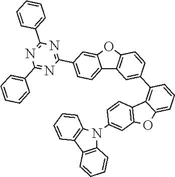 [SMILES: c1ccc(-c2nc(-c3ccccc3)nc(-c3ccc4c(c3)oc3ccc(-c5cccc6oc7cc(-n8c9ccccc9c9ccccc98)ccc7c56)cc34)n2)cc1] |
| 1-111 | | | 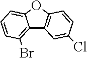 [SMILES: Clc1ccc2oc3cccc(Br)c3c2c1] | | 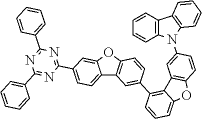 [SMILES: c1ccc(-c2nc(-c3ccccc3)nc(-c3ccc4c(c3)oc3ccc(-c5cccc6oc7ccc(-n8c9ccccc9c9ccccc98)cc7c56)cc34)n2)cc1] |
| 1-214 | | 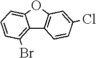 [SMILES: Clc1ccc2c(c1)oc1cccc(Br)c12] | 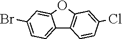 [SMILES: Clc1ccc2c(c1)oc1cc(Br)ccc12] | | 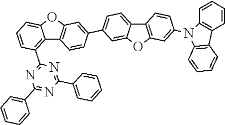 [SMILES: c1ccc(-c2nc(-c3ccccc3)nc(-c3cccc4oc5cc(-c6ccc7c(c6)oc6cc(-n8c9ccccc9c9ccccc98)ccc67)ccc5c34)n2)cc1] |
| 1-215 | | | 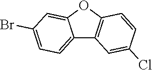 [SMILES: Clc1ccc2oc3cc(Br)ccc3c2c1] | | 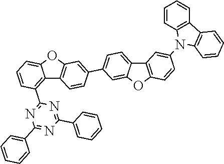 [SMILES: c1ccc(-c2nc(-c3ccccc3)nc(-c3cccc4oc5cc(-c6ccc7c(c6)oc6ccc(-n8c9ccccc9c9ccccc98)cc67)ccc5c34)n2)cc1] |
| 1-222 | | | 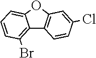 [SMILES: Clc1ccc2c(c1)oc1cccc(Br)c12] | | 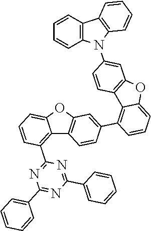 [SMILES: c1ccc(-c2nc(-c3ccccc3)nc(-c3cccc4oc5cc(-c6cccc7oc8cc(-n9c%10ccccc%10c%10ccccc%109)ccc8c67)ccc5c34)n2)cc1] |
| 1-223 | | | 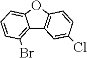 [SMILES: Clc1ccc2oc3cccc(Br)c3c2c1] | | 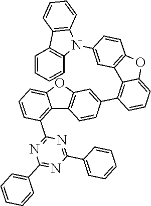 [SMILES: c1ccc(-c2nc(-c3ccccc3)nc(-c3cccc4oc5cc(-c6cccc7oc8ccc(-n9c%10ccccc%10c%10ccccc%109)cc8c67)ccc5c34)n2)cc1] |
| 1-230 | | 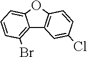 [SMILES: Clc1ccc2oc3cccc(Br)c3c2c1] | 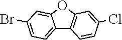 [SMILES: Clc1ccc2c(c1)oc1cc(Br)ccc12] | | 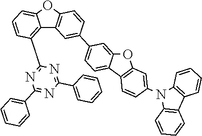 [SMILES: c1ccc(-c2nc(-c3ccccc3)nc(-c3cccc4oc5ccc(-c6ccc7c(c6)oc6cc(-n8c9ccccc9c9ccccc98)ccc67)cc5c34)n2)cc1] |
| 1-231 | | | 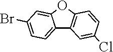 [SMILES: Clc1ccc2oc3cc(Br)ccc3c2c1] | | 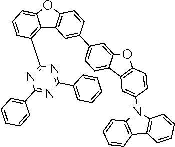 [SMILES: c1ccc(-c2nc(-c3ccccc3)nc(-c3cccc4oc5ccc(-c6ccc7c(c6)oc6ccc(-n8c9ccccc9c9ccccc98)cc67)cc5c34)n2)cc1] |
| 1-238 | | | 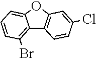 [SMILES: Clc1ccc2c(c1)oc1cccc(Br)c12] | | 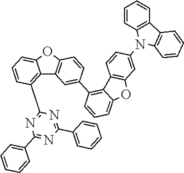 [SMILES: c1ccc(-c2nc(-c3ccccc3)nc(-c3cccc4oc5ccc(-c6cccc7oc8cc(-n9c%10ccccc%10c%10ccccc%109)ccc8c67)cc5c34)n2)cc1] |
| 1-239 | | | 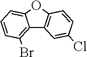 [SMILES: Clc1ccc2oc3cccc(Br)c3c2c1] | | 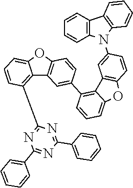 [SMILES: c1ccc(-c2nc(-c3ccccc3)nc(-c3cccc4oc5ccc(-c6cccc7oc8ccc(-n9c%10ccccc%10c%10ccccc%109)cc8c67)cc5c34)n2)cc1] |
| 1-259 | | 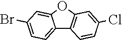 [SMILES: Clc1ccc2c(c1)oc1cc(Br)ccc12] | 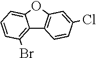 [SMILES: Clc1ccc2c(c1)oc1cccc(Br)c12] | 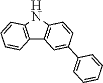 [SMILES: c1ccc(-c2ccc3[nH]c4ccccc4c3c2)cc1] | 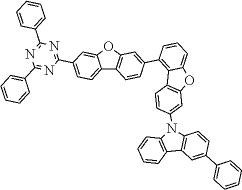 [SMILES: c1ccc(-c2ccc3c(c2)c2ccccc2n3-c2ccc3c(c2)oc2cccc(-c4ccc5c(c4)oc4cc(-c6nc(-c7ccccc7)nc(-c7ccccc7)n6)ccc45)c23)cc1] |
| 1-263 | | 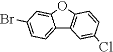 [SMILES: Clc1ccc2oc3cc(Br)ccc3c2c1] | 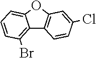 [SMILES: Clc1ccc2c(c1)oc1cccc(Br)c12] | 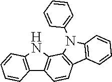 [SMILES: c1ccc(-n2c3ccccc3c3ccc4c5ccccc5[nH]c4c32)cc1] | 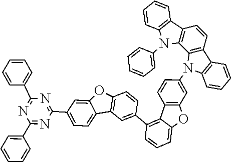 [SMILES: c1ccc(-c2nc(-c3ccccc3)nc(-c3ccc4c(c3)oc3ccc(-c5cccc6oc7cc(-n8c9ccccc9c9ccc%10c%11ccccc%11n(-c%11ccccc%11)c%10c98)ccc7c56)cc34)n2)cc1] |
| 1-264 | | | 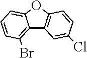 [SMILES: Clc1ccc2oc3cccc(Br)c3c2c1] | 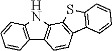 [SMILES: c1ccc2c(c1)[nH]c1c2ccc2c3ccccc3sc21] | 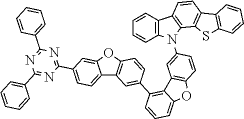 [SMILES: c1ccc(-c2nc(-c3ccccc3)nc(-c3ccc4c(c3)oc3ccc(-c5cccc6oc7ccc(-n8c9ccccc9c9ccc%10c%11ccccc%11sc%10c98)cc7c56)cc34)n2)cc1] |
| 1-267 | | 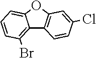 [SMILES: Clc1ccc2c(c1)oc1cccc(Br)c12] | 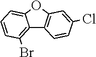 [SMILES: Clc1ccc2c(c1)oc1cccc(Br)c12] | 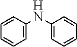 [SMILES: c1ccc(Nc2ccccc2)cc1] | 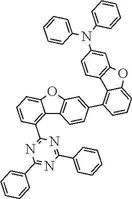 [SMILES: c1ccc(-c2nc(-c3ccccc3)nc(-c3cccc4oc5cc(-c6cccc7oc8cc(N(c9ccccc9)c9ccccc9)ccc8c67)ccc5c34)n2)cc1] |
| 1-270 | 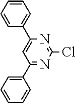 [SMILES: Clc1nc(-c2ccccc2)cc(-c2ccccc2)n1] | 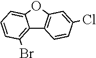 [SMILES: Clc1ccc2c(c1)oc1cccc(Br)c12] | 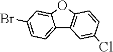 [SMILES: Clc1ccc2oc3cc(Br)ccc3c2c1] | 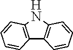 [SMILES: c1ccc2c(c1)[nH]c1ccccc12] | 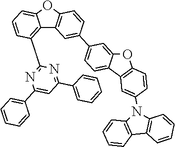 [SMILES: c1ccc(-c2cc(-c3ccccc3)nc(-c3cccc4oc5ccc(-c6ccc7c(c6)oc6ccc(-n8c9ccccc9c9ccccc98)cc67)cc5c34)n2)cc1] |
| 2-11 | 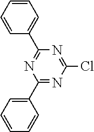 [SMILES: Clc1nc(-c2ccccc2)nc(-c2ccccc2)n1] | 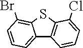 [SMILES: Clc1cccc2c1sc1c(Br)cccc12] | 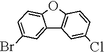 [SMILES: Clc1ccc2oc3ccc(Br)cc3c2c1] | | 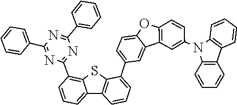 [SMILES: c1ccc(-c2nc(-c3ccccc3)nc(-c3cccc4c3sc3c(-c5ccc6oc7ccc(-n8c9ccccc9c9ccccc98)cc7c6c5)cccc34)n2)cc1] |
| 2-86 | | 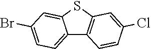 [SMILES: Clc1ccc2c(c1)sc1cc(Br)ccc12] | 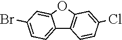 [SMILES: Clc1ccc2c(c1)oc1cc(Br)ccc12] | | 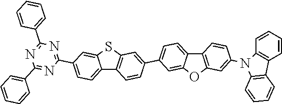 [SMILES: c1ccc(-c2nc(-c3ccccc3)nc(-c3ccc4c(c3)sc3cc(-c5ccc6c(c5)oc5cc(-n7c8ccccc8c8ccccc87)ccc56)ccc34)n2)cc1] |
| 2-87 | | | 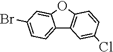 [SMILES: Clc1ccc2oc3cc(Br)ccc3c2c1] | | 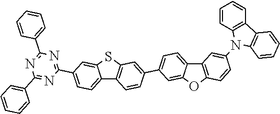 [SMILES: c1ccc(-c2nc(-c3ccccc3)nc(-c3ccc4c(c3)sc3cc(-c5ccc6c(c5)oc5ccc(-n7c8ccccc8c8ccccc87)cc56)ccc34)n2)cc1] |
| 3-102 | | 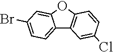 [SMILES: Clc1ccc2oc3cc(Br)ccc3c2c1] | 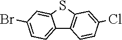 [SMILES: Clc1ccc2c(c1)sc1cc(Br)ccc12] | | 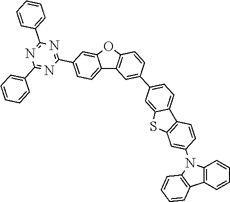 [SMILES: c1ccc(-c2nc(-c3ccccc3)nc(-c3ccc4c(c3)oc3ccc(-c5ccc6c(c5)sc5cc(-n7c8ccccc8c8ccccc87)ccc56)cc34)n2)cc1] |
| 3-214 | | 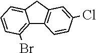 [SMILES: Clc1ccc2c(c1)Cc1cccc(Br)c1-2] | | | 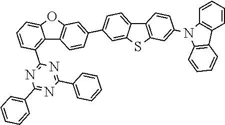 [SMILES: c1ccc(-c2nc(-c3ccccc3)nc(-c3cccc4oc5cc(-c6ccc7c(c6)sc6cc(-n8c9ccccc9c9ccccc98)ccc67)ccc5c34)n2)cc1] |
| 4-230 | | 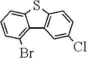 [SMILES: Clc1ccc2sc3cccc(Br)c3c2c1] | | | 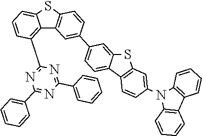 [SMILES: c1ccc(-c2nc(-c3ccccc3)nc(-c3cccc4sc5ccc(-c6ccc7c(c6)sc6cc(-n8c9ccccc9c9ccccc98)ccc67)cc5c34)n2)cc1] |
| 4-239 | | | 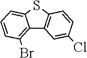 [SMILES: Clc1ccc2sc3cccc(Br)c3c2c1] | | 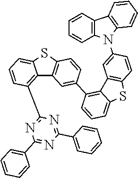 [SMILES: c1ccc(-c2nc(-c3ccccc3)nc(-c3cccc4sc5ccc(-c6cccc7sc8ccc(-n9c%10ccccc%10c%10ccccc%109)cc8c67)cc5c34)n2)cc1] |

### <Preparation Example 2> Preparation of Target Compound 5-2

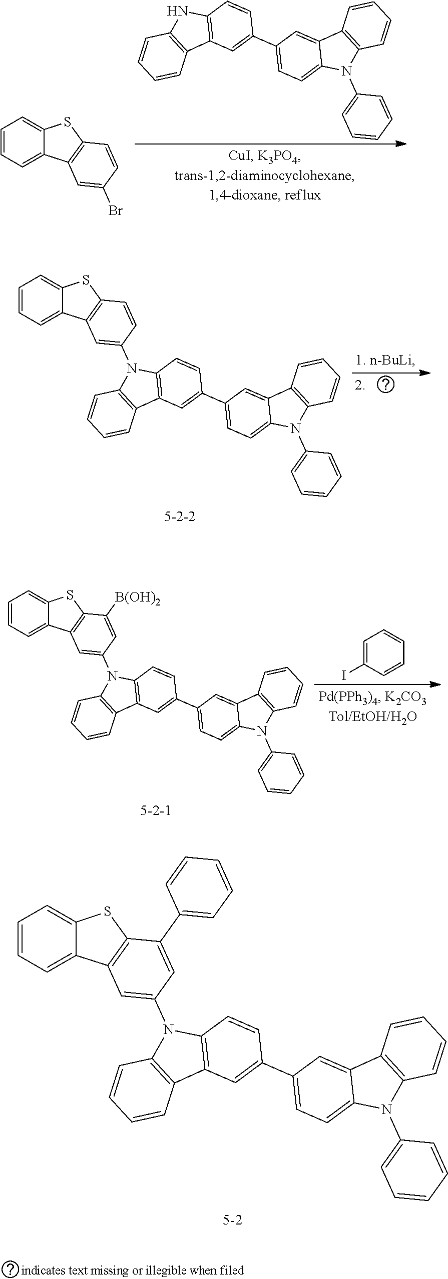 [SMILES: Brc1ccc2sc3ccccc3c2c1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1 ; OB(O)c1cc(-n2c3ccccc3c3cc(-c4ccc5c(c4)c4ccccc4n5-c4ccccc4)ccc32)cc2c1sc1ccccc12 ; Ic1ccccc1 ; c1ccc(-c2cc(-n3c4ccccc4c4cc(-c5ccc6c(c5)c5ccccc5n6-c5ccccc5)ccc43)cc3c2sc2ccccc23)cc1]

### 1) Preparation of Compound 5-2-2

2-Bromodibenzo[b,d]thiophene (4.2 g, 15.8 mM), 9-phenyl-9H,9′H-3,3′-bicarbazole (6.5 g, 15.8 mM), CuI (3.0 g, 15.8 mM), trans-1,2-diaminocyclohexane (1.9 mL, 15.8 mM) and K₃PO₄ (3.3 g, 31.6 mM) were dissolved in 1,4-dioxane (100 mL), and refluxed for 24 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex=1:3), and recrystallized with methanol to obtain Compound 5-2-2 (7.9 g, 85%).

### 2) Preparation of Compound 5-2-1

To a mixture solution of Compound 5-2-1 (8.4 g, 14.3 mMol) and tetrahydrofuran (THF) (100 mL), 2.5 M n-BuLi (7.4 mL, 18.6 mmol) was added dropwise at −78° C., and the result was stirred for 1 hour at room temperature. Trimethyl borate (4.8 mL, 42.9 mmol) was added dropwise to the reaction mixture, and the result was stirred for 2 hours at room temperature. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:MeOH=100:3), and recrystallized with dichloromethane (DCM) to obtain Compound 5-2-1 (3.9 g, 70%). The MeOH means methanol.

### 3) Preparation of Target Compound 5-2

Compound 5-2-1 (6.7 g, 10.5 mM), iodobenzene (2.1 g, 10.5 mM), Pd(PPh₃)₄ (606 mg, 0.52 mM) and K₂CO₃ (2.9 g, 21.0 mM) were dissolved in toluene/ethanol/water (toluene/EtOH/H₂O) (100 mL/20 mL/20 mL), and refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 5-2 (4.9 g, 70%).

### <Preparation Example 3> Preparation of Target Compound 5-3

Target Compound 5-3 (83%) was obtained in the same manner as Preparation of Compound 5-2 of Preparation Example 2 except that 4-iodo-1,1′-biphenyl was used instead of iodobenzene.

### <Preparation Example 4> Preparation of Target Compound 5-12

Target Compound 5-12 (80%) was obtained in the same manner as Preparation of Compound 5-2 of Preparation Example 2 except that 4-iododibenzo[b,d]furan was used instead of iodobenzene.

### <Preparation Example 5> Preparation of Target Compound 6-3

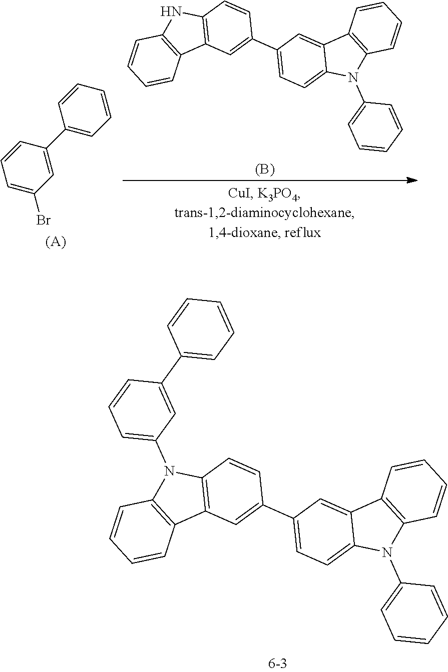

### 1) Preparation of Target Compound 6-3

3-Bromo-1,1′-biphenyl (A) (3.7 g, 15.8 mM), 9-phenyl-9H,9′H-3,3′-bicarbazole (B) (6.5 g, 15.8 mM), CuI (3.0 g, 15.8 mM), trans-1,2-diaminocyclohexane (1.9 mL, 15.8 mM) and K₃PO₄ (3.3 g, 31.6 mM) were dissolved in 1,4-dioxane (100 mL), and refluxed for 24 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex=1:3), and recrystallized with methanol (MeOH) to obtain target Compound 6-3 (7.5 g, 85%).

Target Compounds were synthesized in the same manner as in Preparation Example 5 except that Intermediate A of the following Table 2 was used instead of 3-bromo-1,1′-biphenyl, and Intermediate B of the following Table 2 was used instead of 9-phenyl-9H,9′H-3,3′-bicarbazole.

TABLE 2

| Compound | | | |
|---|---|---|---|
| No. | Intermediate A | Intermediate B | Target Compound |
| 6-4 | 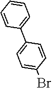 [SMILES: Brc1ccc(-c2ccccc2)cc1] | 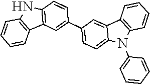 [SMILES: c1ccc(-n2c3ccccc3c3cc(-c4ccc5[nH]c6ccccc6c5c4)ccc32)cc1] | 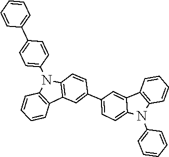 [SMILES: c1ccc(-c2ccc(-n3c4ccccc4c4cc(-c5ccc6c(c5)c5ccccc5n6-c5ccccc5)ccc43)cc2)cc1] |
| 6-7 | 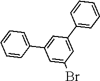 [SMILES: Brc1cc(-c2ccccc2)cc(-c2ccccc2)c1] | | 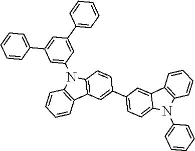 [SMILES: c1ccc(-c2cc(-c3ccccc3)cc(-n3c4ccccc4c4cc(-c5ccc6c(c5)c5ccccc5n6-c5ccccc5)ccc43)c2)cc1] |
| 6-9 | 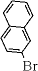 [SMILES: Brc1ccc2ccccc2c1] | | 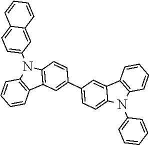 [SMILES: c1ccc(-n2c3ccccc3c3cc(-c4ccc5c(c4)c4ccccc4n5-c4ccc5ccccc5c4)ccc32)cc1] |
| 6-31 | 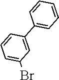 [SMILES: Brc1cccc(-c2ccccc2)c1] | 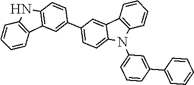 [SMILES: c1ccc(-c2cccc(-n3c4ccccc4c4cc(-c5ccc6[nH]c7ccccc7c6c5)ccc43)c2)cc1] | 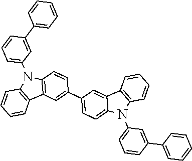 [SMILES: c1ccc(-c2cccc(-n3c4ccccc4c4cc(-c5ccc6c(c5)c5ccccc5n6-c5cccc(-c6ccccc6)c5)ccc43)c2)cc1] |
| 6-32 | 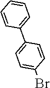 [SMILES: Brc1ccc(-c2ccccc2)cc1] | | 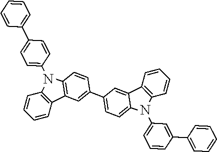 [SMILES: c1ccc(-c2ccc(-n3c4ccccc4c4cc(-c5ccc6c(c5)c5ccccc5n6-c5cccc(-c6ccccc6)c5)ccc43)cc2)cc1] |
| 6-42 | 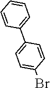 [SMILES: Brc1ccc(-c2ccccc2)cc1] | 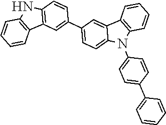 [SMILES: c1ccc(-c2ccc(-n3c4ccccc4c4cc(-c5ccc6[nH]c7ccccc7c6c5)ccc43)cc2)cc1] | 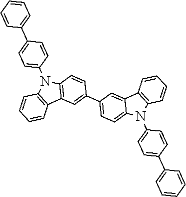 [SMILES: c1ccc(-c2ccc(-n3c4ccccc4c4cc(-c5ccc6c(c5)c5ccccc5n6-c5ccc(-c6ccccc6)cc5)ccc43)cc2)cc1] |

### <Preparation Example 6> Preparation of Target Compound 1-273

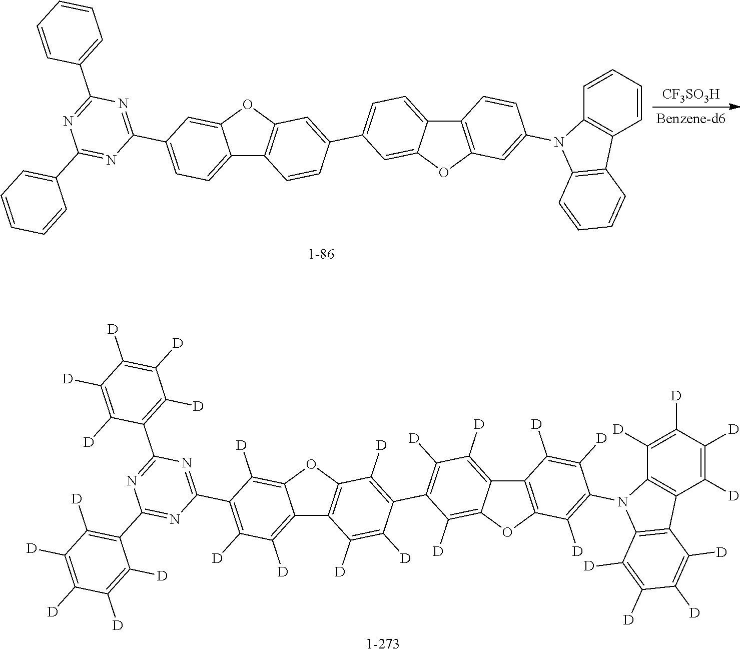

### 1) Preparation of Target Compound 1-273

Compound 1-86 (7.7 g, 10.5 mM) was dissolved in triflic acid (23 mL, 262.5 mM) and benzene-d6 (70 mL), and refluxed at 50° C. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 1-273 (6.2 g, 78%).

### <Preparation Example 7> Preparation of Target Compound 5-105

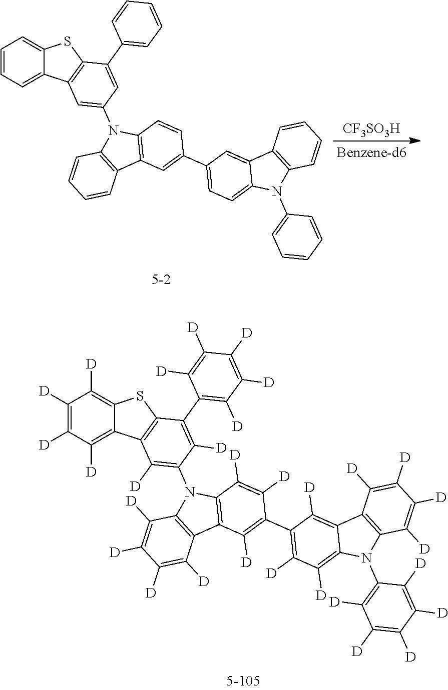

1) Preparation of Target Compound 5-105

Target Compound 5-105 (80%) was obtained in the same manner as in Preparation of Compound 1-273 of Preparation Example 6 except that Compound 5-2 was used instead of Compound 1-86.

### <Preparation Example 8> Preparation of Target Compound 6-99

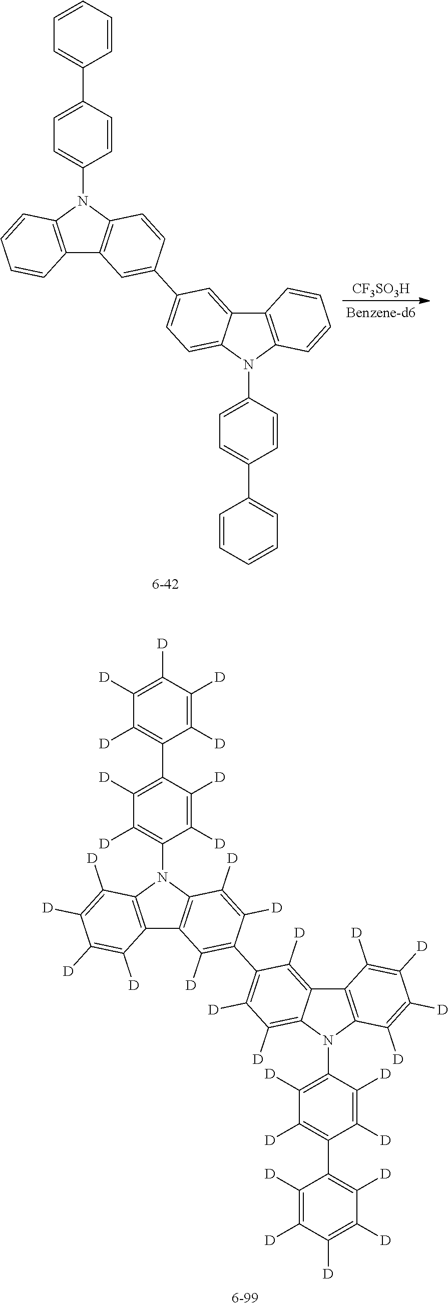

### 1) Preparation of Target Compound 6-99

Target Compound 6-99 (79%) was obtained in the same manner as in Preparation of Compound 1-273 of Preparation Example 6 except that Compound 6-42 was used instead of Compound 1-86.

Compounds other than the compounds described in Preparation Example 1 to Preparation Example 8 and Table 1 and Table 2 were also prepared in the same manner as in the methods described in the preparation examples described above.

Synthesis identification results of the prepared compounds are shown in the following Table 3 and Table 4. The following Table 3 shows measurement values of ¹H NMR (CDCl₃, 200 Mz), and the following Table 4 shows measurement values of FD-mass spectrometry (FD-MS: field desorption mass spectrometry).

TABLE 3

| Compound | |
|---|---|
| No. | ¹H NMR (CDCl₃, 200 Mz) |
| 1-1 | δ = 8.55 (1H, d), 8.36 (4H, m), 8.19 (1H, d), 8.08-8.02 |
| | (5H, m), 7.94-7.88 (3H, m), 7.58-7.46 (12H, m), 7.35- |
| | 7.31 (2H, m), 7.20-7.16 (2H, m) |
| 1-86 | δ = 8.55 (1H, d), 8.36 (4H, m), 8.19 (1H, d), 8.03-7.94 |
| | (5H, m), 7.82-7.76 (6H, m), 7.58-7.50 (9H, m), 7.35 |
| | (1H, t), 7.25-7.16 (3H, m) |
| 1-87 | δ = 8.55 (1H, d), 8.36 (4H, m), 8.19 (1H, d), 8.03 (3H, |
| | d), 7.94 (1H, d), 7.82-7.74 (7H, m), 7.61-7.50 (9H, |
| | m), 7.35-7.31 (2H, m), 7.20-7.16 (2H, m) |
| 1-94 | δ = 8.55 (1H, d), 8.36 (4H, m), 8.19 (1H, d), 8.03-7.94 |
| | (4H, m), 7.82-7.76 (5H, m), 7.69 (1H, m), 7.58-7.50 |
| | (10H, m), 7.35 (1H, t), 7.25-7.16 (3H, m) |
| 1-95 | δ = 8.55 (1H, d), 8.36 (4H, m), 8.19 (1H, d), 8.03 (2H, |
| | m), 7.94 (1H, d), 7.82-7.69 (7H, m), 7.61-7.50 (10H, |
| | m), 7.35-7.31 (2H, m), 7.20-7.16 (2H, m) |
| 1-102 | δ = 8.55 (1H, d), 8.36 (4H, m), 8.19 (1H, d), 8.03-7.76 |
| | (11H, m), 7.58-7.50 (9H, m), 7.35 (1H, t), 7.25-7.16 |
| | (3H, m) |
| 1-103 | δ = 8.55 (1H, d), 8.36 (4H, m), 8.19 (1H, d), 8.03 (2H, |
| | d), 7.94-7.74 (9H, m), 7.61-7.50 (9H, m), 7.35-7.31 |
| | (2H, m), 7.20-7.16 (2H, m) |
| 1-110 | δ = 8.55 (1H, d), 8.36 (4H, m), 8.19 (1H, d), 8.03-7.76 |
| | (9H, m), 7.69 (1H, d), 7.58-7.50 (10H, m), 7.35 (1H, |
| | t), 7.25-7.16 (3H, m) |
| 1-111 | δ = 8.55 (1H, d), 8.36 (4H, m), 8.19 (1H, d), 8.03 (1H, |
| | d), 7.94-7.69 (9H, m), 7.61-7.50 (10H, m), 7.35-7.31 |
| | (2H, m), 7.20-7.16 (2H, m) |
| 1-214 | δ = 8.55 (1H, d), 8.36 (4H, m), 8.19 (1H, d), 8.03-7.94 |
| | (4H, m), 7.82-7.76 (5H, m), 7.69 (1H, m), 7.58-7.50 |
| | (10H, m), 7.35 (1H, t), 7.25-7.16 (3H, m) |
| 1-215 | δ = 8.55 (1H, d), 8.36 (4H, m), 8.19 (1H, d), 8.03 (2H, |
| | d), 7.94 (1H, d), 7.82-7.69 (7H, m), 7.58-7.50 (10H, |
| | m), 7.35-7.31 (2H, m), 7.20-7.16 (2H, m) |
| 1-222 | δ = 8.55 (1H, d), 8.36 (4H, m), 8.19 (1H, d), 8.03-7.94 |
| | (3H, m), 7.82-7.76 (4H, m), 7.69 (2H, d), 7.58-7.50 |
| | (11H, m), 7.35 (1H, t), 7.25-7.16 (3H, m) |
| 1-223 | δ = 8.55 (1H, d), 8.36 (4H, m), 8.19 (1H, d), 8.03 (1H, |
| | d), 7.94 (1H, d), 7.82-7.57 (7H, m), 7.61-7.50 (11H, |
| | m), 7.35-7.31 (2H, m), 7.20-7.16 (2H, m) |
| 1-230 | δ = 8.55 (1H, d), 8.36 (4H, m), 8.19 (1H, d), 8.03-7.76 |
| | (9H, m), 7.69 (1H, d), 7.58-7.50 (10H, m), 7.35 (1H, |
| | m), 7.25-7.16 (3H, m) |
| 1-231 | δ = 8.55 (1H, d), 8.36 (4H, m), 8.19 (1H, d), 8.03 (1H, |
| | d), 7.94-7.69 (9H, m), 7.59-7.50 (10H, m), 7.35-7.31 |
| | (2H, m), 7.20-7.16 (2H, m) |
| 1-238 | δ = 8.55 (1H, d), 8.36 (4H, m), 8.19 (1H, d), 7.98-7.79 |
| | (7H, m), 7.69 (2H, m), 7.58-7.50 (10H, m), 7.35 (1H, |
| | m), 7.25-7.16 (3H, m) |
| 1-239 | δ = 8.55 (1H, d), 8.36 (4H, m), 8.19 (1H, d), 7.94-7.69 |
| | (9H, m), 7.61-7.50 (11H, m), 7.35-7.31 (2H, m), 7.20- |
| | 7.16 (2H, m) |
| 1-259 | δ = 8.55 (1H, d), 8.36 (4H, m), 8.03-7.89 (6H, m), 7.82- |
| | 7.69 (9H, m), 7.57-7.35 (12H, m), 7.25 (1H, d), 7.16 |
| | (1H, d) |
| 1-263 | δ = 8.55 (2H, d), 8.36 (4H, m), 8.12 (1H, d), 8.03-7.76 |
| | (10H, m), 7.69-7.50 (15H, m), 7.35 (2H, t), 7.25 (1H, |
| | d), 7.16 (2H, m) |
| 1-264 | δ = 8.55 (1H, d), 8.45 (1H, d), 8.36 (4H, m), 8.05-8.03 |
| | (2H, m), 7.93-7.69 (10H, m), 7.61-7.49 (11H, m), 7.35- |
| | 7.31 (2H, m), 7.16 (1H, m) |
| 1-267 | δ = 8.36 (4H, m), 8.03 (2H, m), 7.82-7.76 (5H, m), 7.69 |
| | (2H, m), 7.57-7.50 (8H, m), 7.24 (4H, m), 7.08-7.00 |
| | (6H, m) |
| 1-270 | δ = 8.55 (1H, d), 8.23 (1H, s), 8.19 (1H, d), 8.03 (1H, |
| | d), 7.94-7.69 (13H, m), 7.61-7.49 (10H, m), 7.35-7.31 |
| | (2H, m), 7.20-7.16 (2H, m) |
| 2-11 | δ = 8.55 (3H, d), 8.36-8.32 (5H, m), 8.19 (1H, m), 7.94- |
| | 7.70 (8H, m), 7.61-7.50 (9H, m), 7.35-7.31 (2H, m), |
| | 7.20-7.16 (2H, m) |
| 2-86 | δ = 8.55 (1H, d), 8.36 (4H, m), 8.24-8.17 (6H, m), 8.03- |
| | 7.94 (4H, m), 7.82-7.76 (2H, m), 7.58-7.50 (9H, m), |
| | 7.35 (1H, t), 7.25-7.16 (3H, m) |
| 2-87 | δ = 8.55 (1H, d), 8.36 (4H, m), 8.24-8.17 (6H, m), 8.03 |
| | (1H, d), 7.94 (2H, d), 7.82-7.74 (3H, m), 7.61-7.50 |
| | (9H, m), 7.35-7.31 (2H, m), 7.20-7.16 (2H, m) |
| 3-102 | δ = 8.55 (1H, d), 8.36 (4H, m), 8.24-8.17 (5H, m), 8.03- |
| | 7.76 (8H, m), 7.58-7.45 (9H, m), 7.35 (1H, t), 7.20- |
| | 7.16 (2H, m) |
| 3-214 | δ = 8.55 (1H, d), 8.36 (4H, m), 8.24-8.17 (5H, m), 8.03- |
| | 7.94 (3H, m), 7.82-7.76 (3H, m), 7.69 (1H, m), 7.58- |
| | 7.45 (10H, m), 7.35 (1H, t), 7.20-7.16 (2H, m) |
| 4-230 | δ = 8.55 (1H, d), 8.36 (4H, m), 8.24-8.12 (7H, m), 8.03- |
| | 7.94 (5H, m), 7.68 (1H, t), 7.58-7.45 (9H, m), 7.35 |
| | (1H, m), 7.20-7.16 (2H, m) |
| 4-239 | δ = 8.55 (1H, d), 8.36 (4H, m), 8.19-7.90 (11H, m), 7.68 |
| | (2H, t), 7.58-7.35 (10H, m), 7.20-7.16 (2H, m) |
| 5-2 | δ = 8.55(1H, d), 8.45(1H, d), 8.30(1H, d), 8.19(1H, d), |
| | 8.13(1H, d), 8.00~7.89(6H, m), 7.77(2H, m), |
| | 7.62~7.35(15H, m), 7.20~7.16(2H, m) |
| 5-3 | δ = 8.55(1H, d), 8.45(1H, d), 8.30(1H, d), 8.19(1H, d), |
| | 8.13(1H, d), 8.00~7.89(6H, m), 7.77-7.75(4H, m), |
| | 7.62~7.35(13H, m), 7.25~7.16(6H, m) |
| 5-12 | δ = 8.55(1H, d), 8.45(1H, d), 8.30(1H, d), |
| | 8.19~7.89(11H, m), 7.77(2H, m), 7.62~7.31(14H, m), |
| | 7.20~7.16(2H, m) |
| 6-3 | δ = 8.55 (1H, d), 8.30 (1H, d), 8.21-8.13 (3H, m), 7.99- |
| | 7.89 (4H, m), 7.77-7.35 (17H, m), 7.20-7.16 (2H, m) |
| 6-4 | δ = 8.55 (1H, d), 8.30(1H, d), 8.19-8.13(2H, m), 7.99- |
| | 7.89(8H, m), 7.77-7.75 (3H, m), 7.62-7.35 (11H, m), |
| | 7.20-7.16 (2H, m) |
| 6-7 | δ = 8.55 (1H, d), 8.31-8.30 (3H, d), 8.19-8.13 (2H, m), |
| | 7.99-7.89 (5H, m), 7.77-7.75 (5H, m), 7.62-7.35 (14H, |
| | m), 7.20-7.16 (2H, m) |
| 6-9 | δ = 8.55 (1H, d), 8.30 (1H, d), 8.19-8.13 (2H, m), 8.03- |
| | 7.77 (9H, m), 7.62-7.50 (9H, m), 7.36-7.35 (2H, m), |
| | 7.20-7.16 (2H, m) |
| 6-31 | δ = 8.55 (1H, d), 8.30 (1H, d), 8.21-8.13 (4H, m), 7.99- |
| | 7.89 (4H, m), 7.77-7.35 (20H, m), 7.20-7.16 (2H, m) |
| 6-32 | δ = 8.55 (1H, d), 8.30 (1H, d), 8.21-8.13 (3H, m), 7.99- |
| | 7.89 (8H, m), 7.77-7.35 (17H, m), 7.20-7.16 (2H, m) |
| 6-42 | δ = 8.55 (1H, d), 8.30 (1H, d), 8.19 (1H, d), 8.13 (1H, |
| | d), 7.99-7.89 (12H, m), 7.77-7.75 (5H, m), 7.58 (1H, |
| | d), 7.50-7.35 (8H, m), 7.20-7.16 (2H, m) |

TABLE 4

| Compound | FD-MS | Compound | FD-MS |
|---|---|---|---|
| 1-1 | m/z = 730.24 | 1-86 | m/z = 730.24 |
| | (C₅₁H₃₀N₄O₂ = 730.83) | | (C₅₁H₃₀N₄O₂ = 730.83) |
| 1-87 | m/z = 730.24 | 1-94 | m/z = 730.24 |
| | (C₅₁H₃₀N₄O₂ = 730.83) | | (C₅₁H₃₀N₄O₂ = 730.83) |
| 1-95 | m/z = 730.24 | 1-102 | m/z = 730.24 |
| | (C₅₁H₃₀N₄O₂ = 730.83) | | (C₅₁H₃₀N₄O₂ = 730.83) |
| 1-103 | m/z = 730.24 | 1-110 | m/z = 730.24 |
| | (C₅₁H₃₀N₄O₂ = 730.83) | | (C₅₁H₃₀N₄O₂ = 730.83) |
| 1-111 | m/z = 730.24 | 1-214 | m/z = 730.24 |
| | (C₅₁H₃₀N₄O₂ = 730.83) | | (C₅₁H₃₀N₄O₂ = 730.83) |
| 1-215 | m/z = 730.24 | 1-222 | m/z = 730.24 |
| | (C₅₁H₃₀N₄O₂ = 730.83) | | (C₅₁H₃₀N₄O₂ = 730.83) |
| 1-223 | m/z = 730.24 | 1-230 | m/z = 730.24 |
| | (C₅₁H₃₀N₄O₂ = 730.83) | | (C₅₁H₃₀N₄O₂ = 730.83) |
| 1-231 | m/z = 730.24 | 1-238 | m/z = 730.24 |
| | (C₅₁H₃₀N₄O₂ = 730.83) | | (C₅₁H₃₀N₄O₂ = 730.83) |
| 1-239 | m/z = 730.24 | 1-259 | m/z = 806.27 |
| | (C₅₁H₃₀N₄O₂ = 730.83) | | (C₅₇H₃₄N₄O₂ = 806.93) |
| 1-263 | m/z = 895.29 | 1-264 | m/z = 836.22 |
| | (C₆₃H₃₇N₅O₂ = 896.02) | | (C₅₇H₃₂N₄O₂S = 836.97) |
| 1-267 | m/z = 732.25 | 1-270 | m/z = 729.24 |
| | (C₅₁H₃₂N₄O₂ = 732.84) | | (C₅₂H₃₁N₃O₂ = 729.84) |
| 1-273 | m/z = 760.43 | 2-11 | m/z = 746.21 |
| | (C₅₁D₃₀N₄O₂ = 761.01) | | (C₅₁H₃₀N₄OS = 746.89) |
| 2-86 | m/z = 746.21 | 2-87 | m/z = 746.21 |
| | (C₅₁H₃₀N₄OS = 746.89) | | (C₅₁H₃₀N₄OS = 746.89) |
| 3-102 | m/z = 746.21 | 3-214 | m/z = 746.21 |
| | (C₅₁H₃₀N₄OS = 746.89) | | (C₅₁H₃₀N₄OS = 746.89) |
| 4-230 | m/z = 762.19 | 4-239 | m/z = 762.19 |
| | (C₅₁H₃₀N₄S₂ = 762.95) | | (C₅₁H₃₀N₄S₂ = 762.95) |
| 5-2 | m/z = 666.21 | 5-3 | m/z = 742.24 |
| | (C₄₈H₃₀N₂S = 666.84) | | (C₅₄H₃₄N₂S = 742.94) |
| 5-12 | m/z = 756.22 | 5-105 | m/z = 696.40 |
| | (C₅₄H₃₂N₂OS = 756.92) | | (C₄₈D₃₀N₂S = 697.03) |
| 6-3 | m/z = 560.23 | 6-4 | m/z = 560.23 |
| | (C₄₂H₂₈N₂ = 560.70) | | (C₄₂H₂₈N₂ = 560.70) |
| 6-7 | m/z = 636.26 | 6-9 | m/z = 534.21 |
| | (C₄₈H₃₂N₂ = 636.80) | | (C₄₀H₂₆N₂ = 534.66) |
| 6-31 | m/z = 636.26 | 6-32 | m/z = 636.26 |
| | (C₄₈H₃₂N₂ = 636.80) | | (C₄₈H₃₂N₂ = 636.80) |
| 6-42 | m/z = 636.26 | 6-99 | m/z = 668.46 |
| | (C₄₈H₃₂N₂ = 636.80) | | (C₄₈D₃₂N₂ = 668.99) |

### Experimental Example 1

### (1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (UVO) treatment was conducted for 5 minutes using UV in an ultraviolet (UV) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, the compound represented by Chemical Formula 1 of the present disclosure described in the following Table 5 was deposited to 400 Å as a host, and a green phosphorescent dopant [Ir(ppy)₃] was doped by 7 wt % of the deposited thickness of the light emitting layer and deposited. After that, BCP (bathocuproine) was deposited to a thickness of 60 Å as a hole blocking layer, and Alq₃ was deposited to a thickness of 200 Å thereon as an electron transfer layer.

Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device was manufactured (Examples 1 to 29 and Comparative Examples 1 to 8).

Meanwhile, all the organic compounds required to manufacture the organic light emitting device were vacuum sublimation purified under 10⁻⁸ torr to 10⁻⁶ torr for each material to be used in the organic light emitting device manufacture.

### (2) Driving Voltage and Light Emission Efficiency of Organic Light Emitting Device

For each of the organic light emitting devices of Examples 1 to 29 and Comparative Examples 1 to 8 manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Herein, T₉₀ means a lifetime (unit: h, time) taken to become 90% with respect to initial luminance.

Results of measuring driving voltage, light emission efficiency, color (EL color) and lifetime of the organic light emitting devices manufactured according to the present disclosure are as shown in the following Table 5.

TABLE 5

| | Light | | | | |
|---|---|---|---|---|---|
| | Emitting | Driving | | Color | |
| | Layer | Voltage | Efficiency | (EL | Lifetime |
| | Compound | (V) | (cd/A) | color) | (T₉₀) |
| Example 1 | 1-1 | 4.91 | 69.8 | Green | 222 |
| Example 2 | 1-86 | 4.33 | 74.2 | | 405 |
| Example 3 | 1-87 | 4.35 | 79.2 | | 382 |
| Example 4 | 1-94 | 4.66 | 71.1 | | 347 |
| Example 5 | 1-95 | 4.69 | 70.9 | | 271 |
| Example 6 | 1-102 | 4.67 | 71.2 | | 390 |
| Example 7 | 1-103 | 4.66 | 71.2 | | 360 |
| Example 8 | 1-110 | 4.67 | 71.2 | | 277 |
| Example 9 | 1-111 | 4.36 | 78.9 | | 256 |
| Example 10 | 1-214 | 4.45 | 72.8 | | 370 |
| Example 11 | 1-215 | 4.32 | 71.5 | | 289 |
| Example 12 | 1-222 | 4.31 | 79.2 | | 261 |
| Example 13 | 1-223 | 4.38 | 76.4 | | 250 |
| Example 14 | 1-230 | 4.67 | 71.2 | | 356 |
| Example 15 | 1-231 | 4.66 | 71.1 | | 271 |
| Example 16 | 1-238 | 4.66 | 71.2 | | 251 |
| Example 17 | 1-239 | 4.41 | 75.8 | | 249 |
| Example 18 | 1-259 | 4.66 | 71.1 | | 352 |
| Example 19 | 1-263 | 4.35 | 79.2 | | 195 |
| Example 20 | 1-264 | 4.45 | 72.8 | | 198 |
| Example 21 | 1-267 | 4.67 | 71.2 | | 154 |
| Example 22 | 1-270 | 4.42 | 75.7 | | 200 |
| Example 23 | 1-273 | 4.53 | 74.9 | | 505 |
| Example 24 | 2-11 | 4.76 | 69.4 | | 185 |
| Example 25 | 2-86 | 4.36 | 78.9 | | 239 |
| Example 26 | 2-87 | 4.42 | 75.7 | | 237 |
| Example 27 | 3-102 | 4.33 | 75.2 | | 246 |
| Example 28 | 3-214 | 4.13 | 79.2 | | 242 |
| Example 29 | 4-230 | 4.41 | 75.8 | | 225 |
| Example 30 | 4-239 | 4.69 | 77.2 | | 221 |
| Comparative | Ref. 1 | 5.66 | 45.8 | | 48 |
| Example 1 | | | | | |
| Comparative | Ref. 2 | 5.03 | 48.7 | | 59 |
| Example 2 | | | | | |
| Comparative | Ref. 3 | 5.47 | 41.2 | | 57 |
| Example 3 | | | | | |
| Comparative | Ref. 4 | 4.42 | 45.7 | | 47 |
| Example 4 | | | | | |
| Comparative | Ref. 5 | 5.33 | 68.2 | | 73 |
| Example 5 | | | | | |
| Comparative | Ref. 6 | 5.37 | 63.5 | | 82 |
| Example 6 | | | | | |
| Comparative | Ref. 7 | 5.82 | 60.8 | | 68 |
| Example 7 | | | | | |
| Comparative | Ref. 8 | 4.45 | 59.8 | | 78 |
| Example 8 | | | | | |

In addition, Ref. 1 to Ref. 8, comparative compounds used in Comparative Examples 1 to 8, are as follows.

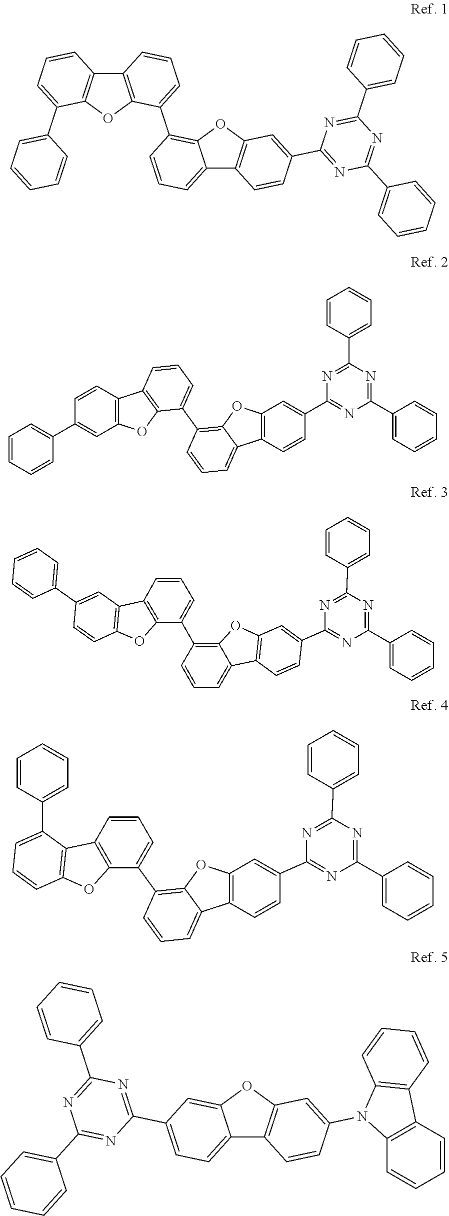 [SMILES: c1ccc(-c2nc(-c3ccccc3)nc(-c3ccc4c(c3)oc3c(-c5cccc6c5oc5c(-c7ccccc7)cccc56)cccc34)n2)cc1 ; c1ccc(-c2ccc3c(c2)oc2c(-c4cccc5c4oc4cc(-c6nc(-c7ccccc7)nc(-c7ccccc7)n6)ccc45)cccc23)cc1 ; c1ccc(-c2ccc3oc4c(-c5cccc6c5oc5cc(-c7nc(-c8ccccc8)nc(-c8ccccc8)n7)ccc56)cccc4c3c2)cc1 ; c1ccc(-c2nc(-c3ccccc3)nc(-c3ccc4c(c3)oc3c(-c5cccc6c5oc5cccc(-c7ccccc7)c56)cccc34)n2)cc1 ; c1ccc(-c2nc(-c3ccccc3)nc(-c3ccc4c(c3)oc3cc(-n5c6ccccc6c6ccccc65)ccc34)n2)cc1]

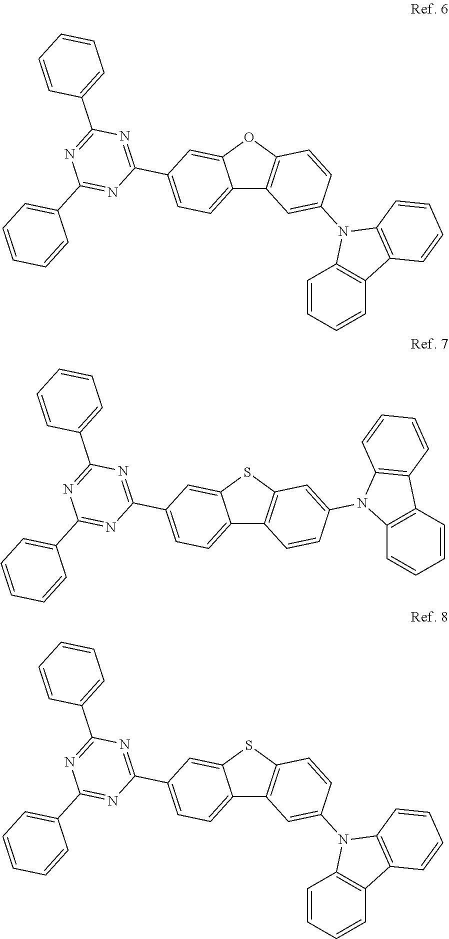

As seen from the results of Table 5, the organic light emitting devices of Examples 1 to 29 using the compound represented by Chemical Formula 1 of the present disclosure as a light emitting layer material of the organic light emitting device had lower driving voltage, enhanced light emission efficiency and significantly improved lifetime compared to Comparative Examples 1 to 8 using the comparative compounds.

In this regard, HOMO orbitals and LUMO orbitals of the compound represented by Chemical Formula 1 of the present disclosure (Compound 1-1) and the comparative compounds Ref. 1 to Ref. 4 are as shown in the following Table 6.

TABLE 6

| | Structural Formula | HOMO Orbital | LUMO Orbital |
|---|---|---|---|
| Ref. 1 | 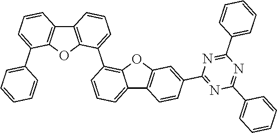 [SMILES: c1ccc(-c2nc(-c3ccccc3)nc(-c3ccc4c(c3)oc3c(-c5cccc6c5oc5c(-c7ccccc7)cccc56)cccc34)n2)cc1] | [symbol] | [symbol] |
| | | −5.78 | −1.96 |
| Ref. 2 | 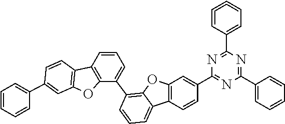 [SMILES: C1=C=CC(c2nc(-c3ccccc3)nc(-c3ccc4c(c3)oc3c(-c5cccc6c5oc5cc(-c7ccccc7)ccc56)cccc34)n2)=CC=1] | [symbol] | [symbol] |
| | | −5.74 | −1.96 |
| Ref. 3 | 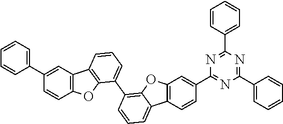 [SMILES: c1ccc(-c2ccc3oc4c(-c5cccc6c5oc5cc(-c7nc(-c8ccccc8)nc(-c8ccccc8)n7)ccc56)cccc4c3c2)cc1] | [symbol] | [symbol] |
| | | −5.73 | −1.96 |
| Ref. 4 | 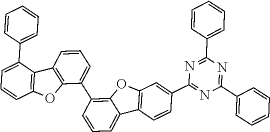 [SMILES: c1ccc(-c2nc(-c3ccccc3)nc(-c3ccc4c(c3)oc3c(-c5cccc6c5oc5cccc(-c7ccccc7)c56)cccc34)n2)cc1] | [symbol] | [symbol] |
| | | −5.78 | −1.94 |
| 1-1 | 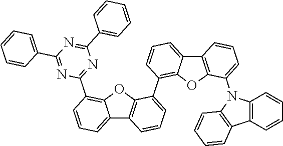 [SMILES: c1ccc(-c2nc(-c3ccccc3)nc(-c3cccc4c3oc3c(-c5cccc6c5oc5c(-n7c8ccccc8c8ccccc87)cccc56)cccc34)n2)cc1] | [symbol] | [symbol] |
| | | −5.40 | −1.84 |

As seen from Table 6, the HOMO orbitals of the comparative compounds Ref. 1, 2, 3 and 4 are delocalized to phenylene and dibenzofuran (DBF), and the HOMO levels are approximately −5.7. However, it is seen that Compound 1-1, the compound represented by Chemical Formula 1 of the present disclosure, is substituted with carbazole having a strong electron pushing ability, and the HOMO level is measured as −5.40. In other words, the compound represented by Chemical Formula 1 of the present disclosure has a higher HOMO level, and when using the compound represented by Chemical Formula 1 of the present disclosure in the light emitting layer of the organic light emitting device, lifetime and efficiency may be enhanced in the electroluminescent device by facilitating hole injection from the host to the dopant.

In addition, HOMO orbitals and LUMO orbitals of the compound represented by Chemical Formula 1 of the present disclosure (Compound 1-86) and the comparative compounds Ref. 5 to Ref. 8 are as shown in the following Table 7.

TABLE 7

| | | |
|---|---|---|
| Structural Formula | HOMO Orbital | LUMO Orbital |

| | | | |
|---|---|---|---|
| Ref. 5 | 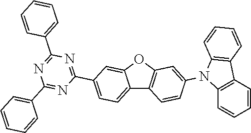 [SMILES: c1ccc(-c2nc(-c3ccccc3)nc(-c3ccc4c(c3)oc3cc(-n5c6ccccc6c6ccccc65)ccc34)n2)cc1] | [symbol] | [symbol] |
| | | −5.38 | −2.06 |
| Ref. 6 | 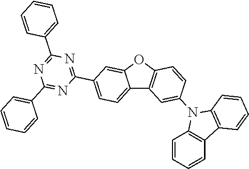 [SMILES: c1ccc(-c2nc(-c3ccccc3)nc(-c3ccc4c(c3)oc3ccc(-n5c6ccccc6c6ccccc65)cc34)n2)cc1] | [symbol] | [symbol] |
| | | −5.36 | −2.08 |
| Ref .7 | 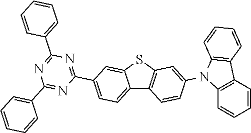 [SMILES: c1ccc(-c2nc(-c3ccccc3)nc(-c3ccc4c(c3)sc3cc(-n5c6ccccc6c6ccccc65)ccc34)n2)cc1] | [symbol] | [symbol] |
| | | −5.39 | −2.08 |
| Ref. 8 | 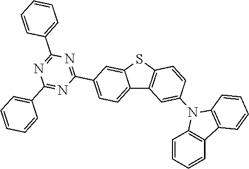 [SMILES: c1ccc(-c2nc(-c3ccccc3)nc(-c3ccc4c(c3)sc3ccc(-n5c6ccccc6c6ccccc65)cc34)n2)cc1] | [symbol] | [symbol] |
| | | −5.37 | −2.09 |
| 1-86 | 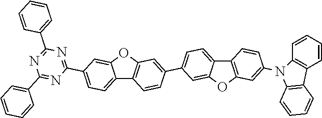 [SMILES: c1ccc(-c2nc(-c3ccccc3)nc(-c3ccc4c(c3)oc3cc(-c5ccc6c(c5)oc5cc(-n7c8ccccc8c8ccccc87)ccc56)ccc34)n2)cc1] | [symbol] | [symbol] |
| | | −5.34 | −2.07 |

As seen from Table 7, the LUMO orbitals of the comparative compounds Ref. 5, 6, 7 and 8 are delocalized from triazine to one heteroring. However, the LUMO orbital of Compound 1-86, the compound represented by Chemical Formula 1 of the present disclosure, is delocalized from triazine to two heterorings. Due to such an expansion of the LUMO orbital, efficiency and lifetime of the organic light emitting device may be enhanced when using the compound represented by Chemical Formula 1 of the present disclosure in the light emitting layer of the organic light emitting device by enhancing electron mobility and stabilizing electrons.

In addition, HOMO orbitals and LUMO orbitals of Compound 1-1, Compound 2-11, Compound 1-86 and Compound 2-87, which are the compounds represented by Chemical Formula 1 of the present disclosure, are as shown in the following Table 8.

TABLE 8

| | Structural Formula | HOMO Orbital | LUMO Orbital |
|---|---|---|---|
| 1-1 | 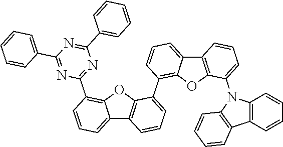 [SMILES: c1ccc(-c2nc(-c3ccccc3)nc(-c3cccc4c3oc3c(-c5cccc6c5oc5c(-n7c8ccccc8c8ccccc87)cccc56)cccc34)n2)cc1] | [symbol] | [symbol] |
| | | −5.40 | −1.84 |
| 2-11 | 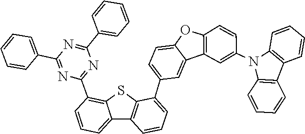 [SMILES: c1ccc(-c2nc(-c3ccccc3)nc(-c3cccc4c3sc3c(-c5ccc6oc7ccc(-n8c9ccccc9c9ccccc98)cc7c6c5)cccc34)n2)cc1] | [symbol] | [symbol] |
| | | −5.34 | −1.95 |
| 1-86 | 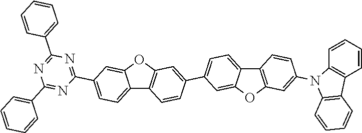 [SMILES: c1ccc(-c2nc(-c3ccccc3)nc(-c3ccc4c(c3)oc3cc(-c5ccc6c(c5)oc5cc(-n7c8ccccc8c8ccccc87)ccc56)ccc34)n2)cc1] | [symbol] | [symbol] |
| | | −5.34 | −2.07 |
| 2-87 | 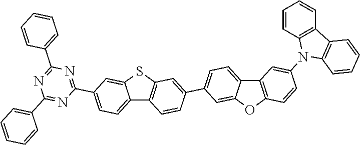 [SMILES: c1ccc(-c2nc(-c3ccccc3)nc(-c3ccc4c(c3)sc3cc(-c5ccc6c(c5)oc5ccc(-n7c8ccccc8c8ccccc87)cc56)ccc34)n2)cc1] | [symbol] | [symbol] |
| | | −5.35 | −2.10 |

Generally, a host in a light emitting layer is a layer to which both holes and electrons are injected, and efficiency and lifetime change depending on stability and mobility of holes and electrons.

The compound represented by Chemical Formula 1 of the present disclosure is a compound formed with triazine that pulls electrons, carbazole that pushes electrons, and two heterorings connecting a donor and an acceptor.

The compound represented by Chemical Formula 1 may change mobility and stability of holes and electrons by changing a bonding position of the donor and the acceptor on the heteroring.

Specifically, carbazole or amine substituting at the end of the compound represented by Chemical Formula 1 has orbital disconnection around N, and accordingly, the HOMO orbital is generally localized to the carbazole or the amine. Herein, the HOMO orbital may be widely delocalized by changing the bonding position, which serves to increase hole mobility and stability.

In addition, in the compound represented by Chemical Formula 1, a heterocyclic group comprising N such as triazine is substituted with dibenzofuran or dibenzothiophene that may affect the LUMO orbital of the heterocyclic group comprising N.

As a result, the LUMO level of the compound may be adjusted depending on the position of substitution of the heterocyclic group comprising N substituted with dibenzofuran or dibenzothiophene. In other words, it means that the LUMO level may be adjusted depending on which position of the dibenzofuran or dibenzothiophene substituting the heterocyclic group comprising N such as triazine.

This means that, by properly adjusting the LUMO level depending on dopant and electron transfer layer (ETL) materials, the compound represented by Chemical Formula 1 of the present application may be used as a material of an organic light emitting device, particularly a material of a light emitting layer, and more specifically a host material. In other words, when changing dopant and electron transfer layer materials, a host material needs to be changed depending on the charge balance.

Specifically, when using a compound with a low LUMO level as a material of a light emitting layer, electrons may be readily injected from a host to a dopant, and when using a compound with a high LUMO level as a material of a light emitting layer, electrons may be readily injected from an electron transfer layer (ETL) to a host.

Particularly, in Compound 1-86, triazine bonds to the No. 3 position of dibenzofuran, and carbazole bonds to the No. 3 position of dibenzofuran, and the LUMO and HOMO orbitals are relatively widely delocalized to the heteroring. It may be identified that this enhances mobility and stability of holes and electrons resulting in an enhanced lifetime.

The above-described result may lead to a favorable result when electron mobility is fast from an electrode to a light emitting layer in an electroluminescent device. However, the result of a host in the light emitting layer may vary depending on the balance between holes and electrons in the device.

Generally, a charge balance between holes and electrons in a light emitting layer changes depending on the tendency of a phosphorescent dopant, and mobility of holes and electrons may be adjusted accordingly by either using a hole transfer auxiliary layer (G′ HTL), a hole blocking layer (HBL) or an electron blocking layer (EBL), or doping to a hole transfer layer (HTL) or an electron transfer layer (ETL). Herein, a device lifetime may decrease when holes and electrons are over-accumulated in the light emitting layer.

When a compound having a structure disconnecting a heterocyclic group comprising N and carbazole or amine as the compound corresponding to the compound of the present application is used as a material of a light emitting layer, mobility of holes and electrons may be lowered to perform a role of decreasing the amounts of holes and electrons accumulated in the light emitting layer, and as a result, a device lifetime may be enhanced.

In addition, dibenzofuran or dibenzothiophene substituted with amine or carbazole as in the compound of the present application affects the LUMO orbital widely spread in the heterocyclic group comprising N, which may affect enhancement in the electron mobility and stability.

### Experimental Example 2

### (1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (UVO) treatment was conducted for 5 minutes using UV in an ultraviolet (UV) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, one type of the compound represented by Chemical Formula 1 of the present disclosure (N-type) and one type of the compound represented by Chemical Formula 13 or 14 of the present disclosure (P-type), or the compound represented by Chemical Formula 1 of the present disclosure (N-type) and one type of the comparative compound (P-type) were pre-mixed as described in the following Table 9 and deposited to 400 Å in one source of supply as a host, and a green phosphorescent dopant [Ir(ppy)₃] was doped by an amount of 7 wt % of the deposited thickness of the light emitting layer and deposited. After that, BCP (bathocuproine) was deposited to 60 Å as a hole blocking layer, and Alq₃ was deposited to a thickness of 200 Å thereon as an electron transfer layer.

Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device was manufactured (Examples 30 to 54 and Comparative Examples 9 to 20).

Meanwhile, all the organic compounds required to manufacture the OLED device were vacuum sublimation purified under 10⁻⁸ torr to 10⁻⁶ torr for each material to be used in the OLED manufacture.

### (2) Driving Voltage and Light Emission Efficiency of Organic Light Emitting Device

For each of the organic light emitting devices of Examples 30 to 54 and Comparative Examples 9 to 20 manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Herein, T₉₀ means a lifetime (unit: h, time) taken to become 90% with respect to initial luminance.

Results of measuring driving voltage, light emission efficiency, color coordinate (CIE) and lifetime of the organic light emitting devices manufactured according to the present disclosure are as shown in the following Table 9.

TABLE 9

| | Light Emitting | | Driving Voltage | Efficiency | Color | Lifetime |
|---|---|---|---|---|---|---|
| | Layer Compound | Ratio | (V) | (cd/A) | (EL Color) | (T₉₀) |
| Example 31 | 1-86:5-2 | 1:8 | 4.73 | 54.2 | Green | 338 |
| Example 32 | | 1:5 | 4.71 | 57.2 | | 444 |
| Example 33 | | 1:2 | 4.33 | 75.2 | | 679 |
| Example 34 | | 1:1 | 4.48 | 70.2 | | 546 |
| Example 35 | | 2:1 | 4.69 | 69.2 | | 525 |
| Example 36 | | 5:1 | 4.32 | 68.3 | | 433 |
| Example 37 | | 8:1 | 4.21 | 67.0 | | 325 |
| Example 38 | 1-87:5-3 | 1:2 | 4.35 | 79.2 | | 599 |
| Example 39 | | 1:1 | 4.41 | 75.8 | | 591 |
| Example 40 | | 2:1 | 4.67 | 71.2 | | 540 |
| Example 41 | 1-94:5-12 | 1:2 | 4.38 | 76.4 | | 539 |
| Example 42 | | 1:1 | 4.45 | 72.8 | | 522 |
| Example 43 | | 2:1 | 4.66 | 71.1 | | 500 |
| Example 44 | 1-86:5-105 | 1:2 | 4.54 | 76.3 | | 779 |
| Example 45 | | 1:1 | 4.69 | 71.3 | | 646 |
| Example 46 | | 2:1 | 4.90 | 70.2 | | 625 |
| Example 47 | 1-102:6-4 | 1:2 | 4.33 | 75.2 | | 620 |
| Example 48 | | 1:1 | 4.48 | 70.2 | | 582 |
| Example 49 | | 2:1 | 4.69 | 69.2 | | 543 |
| Example 50 | 1-103:6-7 | 1:2 | 4.33 | 75.2 | | 569 |
| Example 51 | | 1:1 | 4.48 | 70.2 | | 543 |
| Example 52 | | 2:1 | 4.69 | 69.2 | | 518 |
| Example 53 | 1-214:6-31 | 1:2 | 4.31 | 79.2 | | 586 |
| Example 54 | | 1:1 | 4.42 | 75.7 | | 558 |
| Example 55 | | 2:1 | 4.66 | 71.1 | | 536 |
| Example 56 | 1-230:6-32 | 1:2 | 4.32 | 74.2 | | 562 |
| Example 57 | | 1:1 | 4.42 | 72.2 | | 553 |
| Example 58 | | 2:1 | 4.66 | 71.2 | | 528 |
| Example 59 | 2-86:6-31 | 1:2 | 4.44 | 74.1 | | 409 |
| Example 60 | | 1:1 | 4.59 | 69.1 | | 371 |
| Example 61 | | 2:1 | 4.80 | 68.0 | | 332 |
| Example 62 | 3-102:6-31 | 1:2 | 4.41 | 74.1 | | 358 |
| Example 63 | | 1:1 | 4.59 | 69.2 | | 332 |
| Example 64 | | 2:1 | 4.78 | 68.2 | | 307 |
| Example 65 | 4-230:6-31 | 1:2 | 4.42 | 78.1 | | 275 |
| Example 66 | | 1:1 | 4.53 | 74.6 | | 246 |
| Example 67 | | 2:1 | 4.76 | 70.0 | | 225 |
| Example 68 | 4-230:6-31 | 1:2 | 4.62 | 79.0 | | 376 |
| Example 69 | | 1:1 | 4.73 | 75.2 | | 343 |
| Example 70 | | 2:1 | 4.96 | 71.8 | | 321 |
| Comparative | 1-86:Ref. 9 | 1:2 | 5.73 | 54.2 | | 316 |
| Example 9 | | | | | | |
| Comparative | | 1:1 | 5.71 | 57.2 | | 225 |
| Example 10 | | | | | | |
| Comparative | | 2:1 | 5.81 | 59.2 | | 133 |
| Example 11 | | | | | | |
| Comparative | 1-86:Ref. 10 | 1:2 | 5.42 | 55.7 | | 399 |
| Example 12 | | | | | | |
| Comparative | | 1:1 | 5.21 | 57.0 | | 311 |
| Example 13 | | | | | | |
| Comparative | | 2:1 | 5.65 | 59.2 | | 240 |
| Example 14 | | | | | | |
| Comparative | 1-86:Ref. 11 | 1:2 | 5.41 | 55.8 | | 464 |
| Example 15 | | | | | | |
| Comparative | | 1:1 | 5.53 | 55.6 | | 331 |
| Example 16 | | | | | | |
| Comparative | | 2:1 | 5.58 | 50.5 | | 310 |
| Example 17 | | | | | | |
| Comparative | 1-86:Ref. 12 | 1:2 | 5.32 | 50.4 | | 138 |
| Example 18 | | | | | | |
| Comparative | | 1:1 | 5.69 | 49.2 | | 79 |
| Example 19 | | | | | | |
| Comparative | | 2:1 | 5.61 | 41.3 | | 44 |
| Example 20 | | | | | | |

In addition, Ref. 9 to Ref. 12, comparative compounds used in Comparative Examples 9 to 20, are as follows.

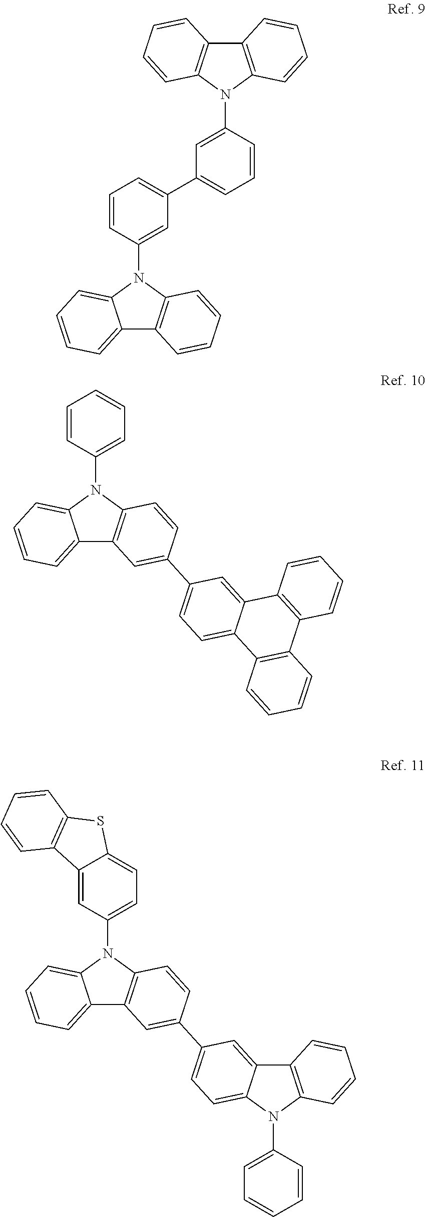

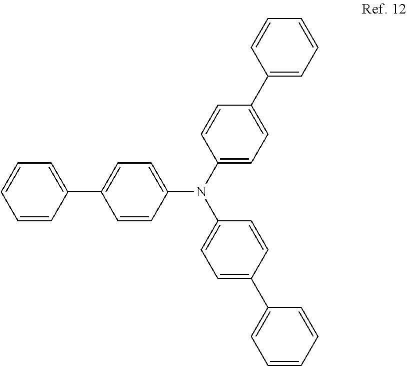 [SMILES: c1ccc(-c2ccc(N(c3ccc(-c4ccccc4)cc3)c3ccc(-c4ccccc4)cc3)cc2)cc1]

From the results of Table 9, effects of more superior efficiency and lifetime are obtained when including the compound represented by Chemical Formula 1 of the present disclosure (N-type) and the compound represented by Chemical Formula 13 or 14 of the present disclosure (P-type) at the same time. Such results may lead to a forecast that an exciplex phenomenon occurs when including the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may be lowered, which resultantly helps with enhancement in the lifetime.

In the present disclosure, it was identified that excellent device properties were obtained when using the compound represented Chemical Formula 13 or 14 performing a donor role and the compound represented by Chemical Formula 1 performing an acceptor role as a light emitting layer host.

In this regard, HOMO orbitals and LUMO orbitals of the compound represented by Chemical Formula 13 of the present disclosure (Compound 5-2), the compound represented by Chemical Formula 13 of the present disclosure (Compound 6-31), and the comparative compounds Ref. 9 to Ref. 12 are as shown in the following Table 10.

TABLE 10

| | Structural Formula | HOMO Orbital | LUMO Orbital |
|---|---|---|---|
| Ref. 9 | 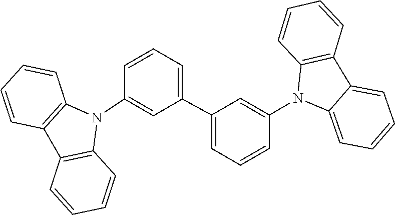 [SMILES: c1cc(-c2cccc(-n3c4ccccc4c4ccccc43)c2)cc(-n2c3ccccc3c3ccccc32)c1] | [symbol] | [symbol] |
| | | −5.40 | −1.19 |
| Ref. 10 | 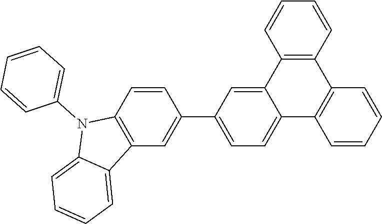 [SMILES: c1ccc(-n2c3ccccc3c3cc(-c4ccc5c6ccccc6c6ccccc6c5c4)ccc32)cc1] | [symbol] | [symbol] |
| | | −5.21 | −1.03 |
| Ref. 11 | 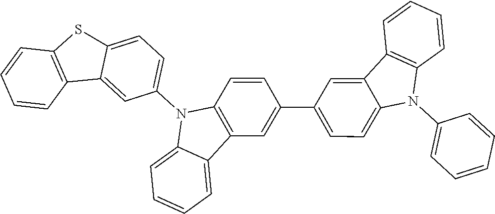 [SMILES: c1ccc(-n2c3ccccc3c3cc(-c4ccc5c(c4)c4ccccc4n5-c4ccc5sc6ccccc6c5c4)ccc32)cc1] | [symbol] | [symbol] |
| | | −4.97 | −1.20 |
| Ref. 12 | 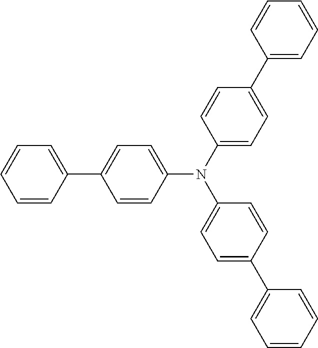 [SMILES: c1ccc(-c2ccc(N(c3ccc(-c4ccccc4)cc3)c3ccc(-c4ccccc4)cc3)cc2)cc1] | [symbol] | [symbol] |
| | | −4.87 | −0.91 |
| 5-2 | 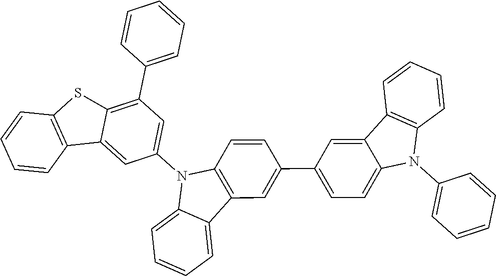 [SMILES: c1ccc(-c2cc(-n3c4ccccc4c4cc(-c5ccc6c(c5)c5ccccc5n6-c5ccccc5)ccc43)cc3c2sc2ccccc23)cc1] | [symbol] | [symbol] |
| | | −4.96 | −1.17 |
| 6-31 | 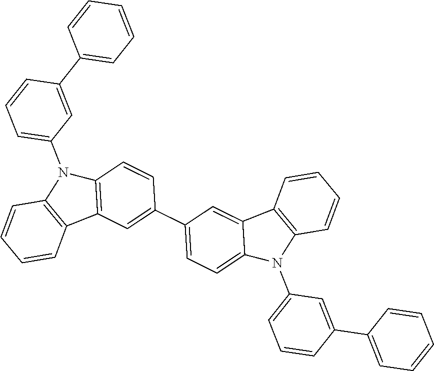 [SMILES: c1ccc(-c2cccc(-n3c4ccccc4c4cc(-c5ccc6c(c5)c5ccccc5n6-c5cccc(-c6ccccc6)c5)ccc43)c2)cc1] | [symbol] | [symbol] |
| | | −4.96 | −1.00 |

As seem from Table 10, the HOMO orbital of the comparative compound Ref. 9 is localized to two carbazoles at the end resulting in low HOMO level and hole mobility. This causes an imbalance between holes and electrons in the device resulting in a decrease in the lifetime.

The HOMO orbital of the comparative compound Ref. 10 is delocalized to carbazole and triphenylene resulting in a higher HOMO level and enhanced hole mobility compared to Ref. 9, however, an imbalance in the device still exists.

When there is no aryl substituent at the No. 4 position of the dibenzothiophene (DBT) core as in the comparative compound Ref 11, molecular stability decreases since a protective substituent is not present at the No. 4 position of the dibenzothiophene (DBT) core that has favorable reactivity, and the LUMO orbital is localized to the dibenzothiophene (DBT) core compared to Compound 5-2, and as a result, the lifetime decreases by failing to effectively stabilize electrons.

Using amine as the p-type as in the comparative compound Ref. 12 causes an imbalance in the device due to too high HOMO level and fast hole mobility, and a decrease in the lifetime is identified.

The HOMO levels of Compound 5-2 represented by Chemical Formula 13 of the present disclosure and Compound 6-31 represented by Chemical Formula 14 of the present disclosure are widely delocalized to two carbazoles. When the compound represented by Chemical Formula 1 of the present disclosure with the compound represented by Chemical Formula 13 of the present disclosure or the compound represented by Chemical Formula 14 of the present disclosure are used at the same time in an organic light emitting device, HOMO level, and hole mobility and stability increase compared to basic carbazole, and a lifetime of the organic light emitting device may be enhanced. Particularly, the structure as Compound 5-2 effectively stabilizes electrons and enhances a lifetime of an organic light emitting device by introducing a dibenzothiophene (DBT) core and an aryl group considering electron stability.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

1. A heterocyclic compound represented by the following Chemical Formula 1: 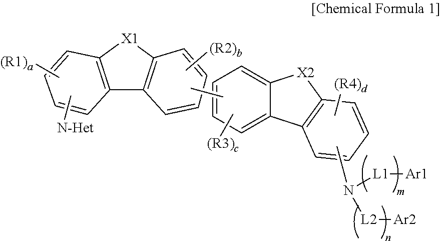
wherein, in Chemical Formula 1,
X1 and X2 are the same as or different from each other, and each independently O; or S;
L1 and L2 are the same as or different from each other, and each independently a direct bond; a substituted or unsubstituted arylene group having 6 to 60 carbon atoms; or a substituted or unsubstituted heteroarylene group having 2 to 60 carbon atoms, and m and n are each independently 0 or 1;
Ar1 and Ar2 are the same as or different from each other, and each independently selected from the group consisting of a substituted or unsubstituted aryl group having 6 to 60 carbon atoms and a substituted or unsubstituted heteroaryl group having 2 to 60 carbon atoms, or two or more groups adjacent to each other bond to each other to form a substituted or unsubstituted aliphatic or aromatic hydrocarbon ring or heteroring;
R1 to R4 are the same as or different from each other, and each independently hydrogen; deuterium; a substituted or unsubstituted aryl group having 6 to 60 carbon atoms; or a substituted or unsubstituted heteroaryl group having 6 to 60 carbon atoms, a to d are each an integer of 0 to 3, and when a to d are each 2 or greater, substituents in the parentheses are the same as each other; and
N-Het is a monocyclic or polycyclic heterocyclic group having 1 to 60 carbon atoms substituted or unsubstituted, and comprising one or more Ns.

2. The heterocyclic compound of claim 1, wherein the substituted or unsubstituted means being substituted with one or more substituents selected from the group consisting of deuterium; a cyano group; linear or branched alkyl having 1 to 60 carbon atoms; linear or branched alkenyl having 2 to 60 carbon atoms; linear or branched alkynyl having 2 to 60 carbon atoms; monocyclic or polycyclic cycloalkyl having 3 to 60 carbon atoms; monocyclic or polycyclic heterocycloalkyl having 2 to 60 carbon atoms; monocyclic or polycyclic aryl having 6 to 60 carbon atoms; monocyclic or polycyclic heteroaryl having 2 to 60 carbon atoms; —SiRR′R″; —P(═O)RR′; alkylamine having 1 to 20 carbon atoms; monocyclic or polycyclic arylamine having 6 to 60 carbon atoms; and monocyclic or polycyclic heteroarylamine having 2 to 60 carbon atoms, or being unsubstituted, or being substituted with a substituent linking two or more substituents selected from among the substituents illustrated above, or being unsubstituted, and R, R′ and R″ are the same as or different from each other and each independently substituted or unsubstituted alkyl having 1 to 60 carbon atoms; substituted or unsubstituted aryl having 6 to 60 carbon atoms; or substituted or unsubstituted heteroaryl having 2 to 60 carbon atoms.

3. The heterocyclic compound of claim 1, wherein Chemical Formula 1 is represented by any one of the following Chemical Formulae 1-1 to 1-4: 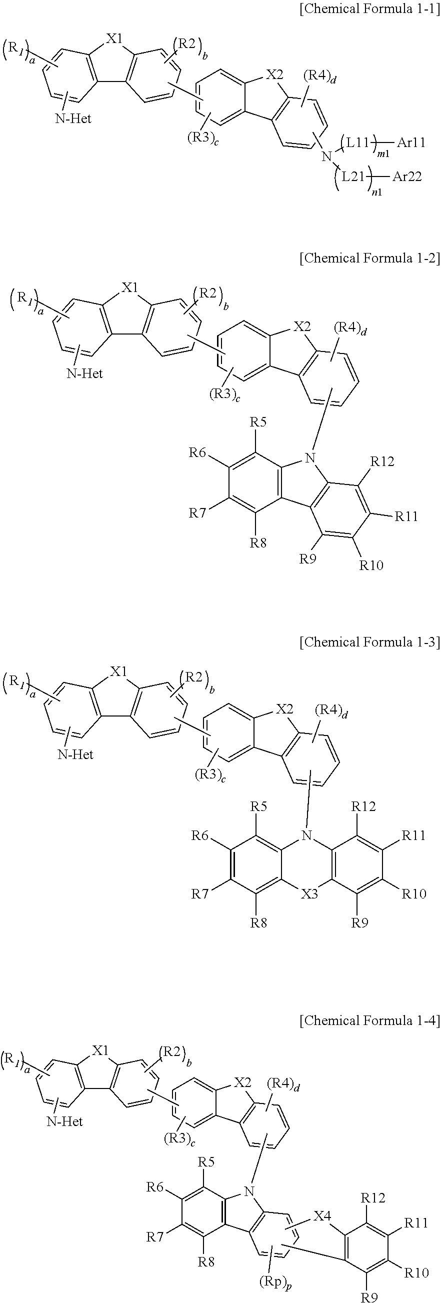
in Chemical Formulae 1-1 to 1-4,
L11 and L21 are the same as or different from each other, and each independently a direct bond; a substituted or unsubstituted arylene group having 6 to 60 carbon atoms; or a substituted or unsubstituted heteroarylene group having 2 to 60 carbon atoms, and m1 and n1 are each independently 0 or 1;
Ar11 and Ar21 are the same as or different from each other, and each independently a substituted or unsubstituted aryl group having 6 to 60 carbon atoms; or a substituted or unsubstituted heteroaryl group having 2 to 60 carbon atoms;
X3 and X4 are each independently O; S; CRaRb; or NRc, Ra and Rb are the same as or different from each other and each independently a substituted or unsubstituted alkyl group having 1 to 10 carbon atoms; or a substituted or unsubstituted aryl group having 6 to 20 carbon atoms, and Rc is a substituted or unsubstituted aryl group having 6 to 20 carbon atoms;
R5 to R12 and Rp are the same as or different from each other, and each independently hydrogen; deuterium; a halogen group; a cyano group; a substituted or unsubstituted aryl group having 6 to 20 carbon atoms; or a substituted or unsubstituted heteroaryl group having 2 to 20 carbon atoms, p is an integer of 0 to 2, and when p is 2, substituents in the parentheses are the same as each other; and
X1, X2, R1 to R4, N-Het and a to d have the same definitions as in Chemical Formula 1.

4. The heterocyclic compound of claim 1, wherein N-Het is a group represented by any one of the following Chemical Formulae 2 to 4: 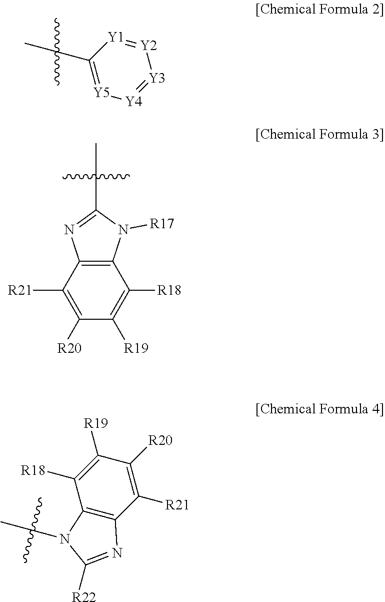
in Chemical Formulae 2 to 4,
Y1 is CR12 or N, Y2 is CR13 or N, Y3 is CR14 or N, Y4 is CR15 or N, Y5 is CR16 or N, and at least one of Y1 to Y5 is N; and
R12 to R16 and R17 to R22 are the same as or different from each other, and each independently selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more groups adjacent to each other bond to each other to form a substituted or unsubstituted aliphatic or aromatic hydrocarbon ring or heteroring, and 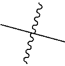
is a site linked to Chemical Formula 1.

5. The heterocyclic compound of claim 1, wherein the heterocyclic compound represented by Chemical Formula 1 has a deuterium content of 0% to 100%.

6. The heterocyclic compound of claim 1, wherein Chemical Formula 1 is represented by any one of the following compounds:

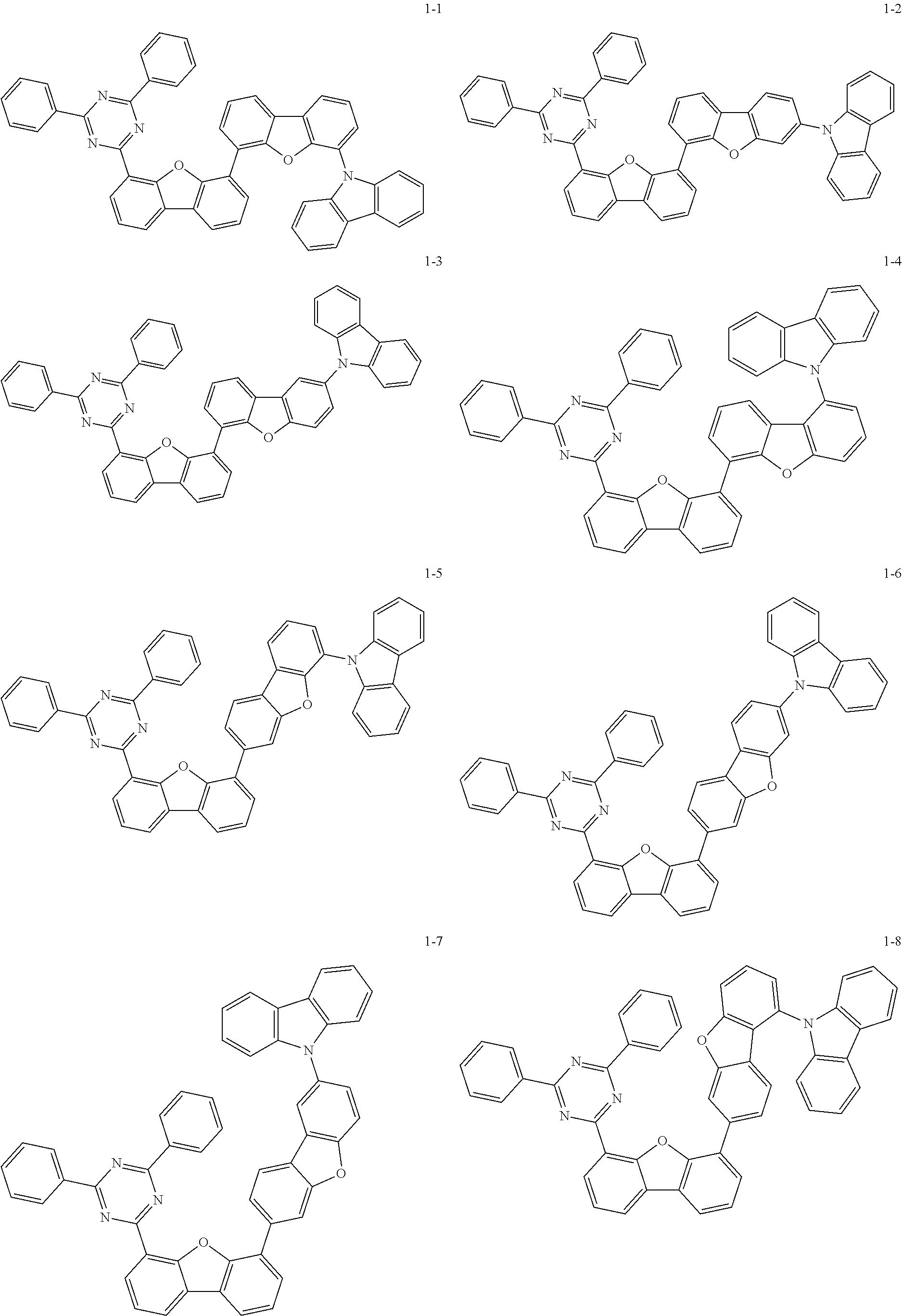

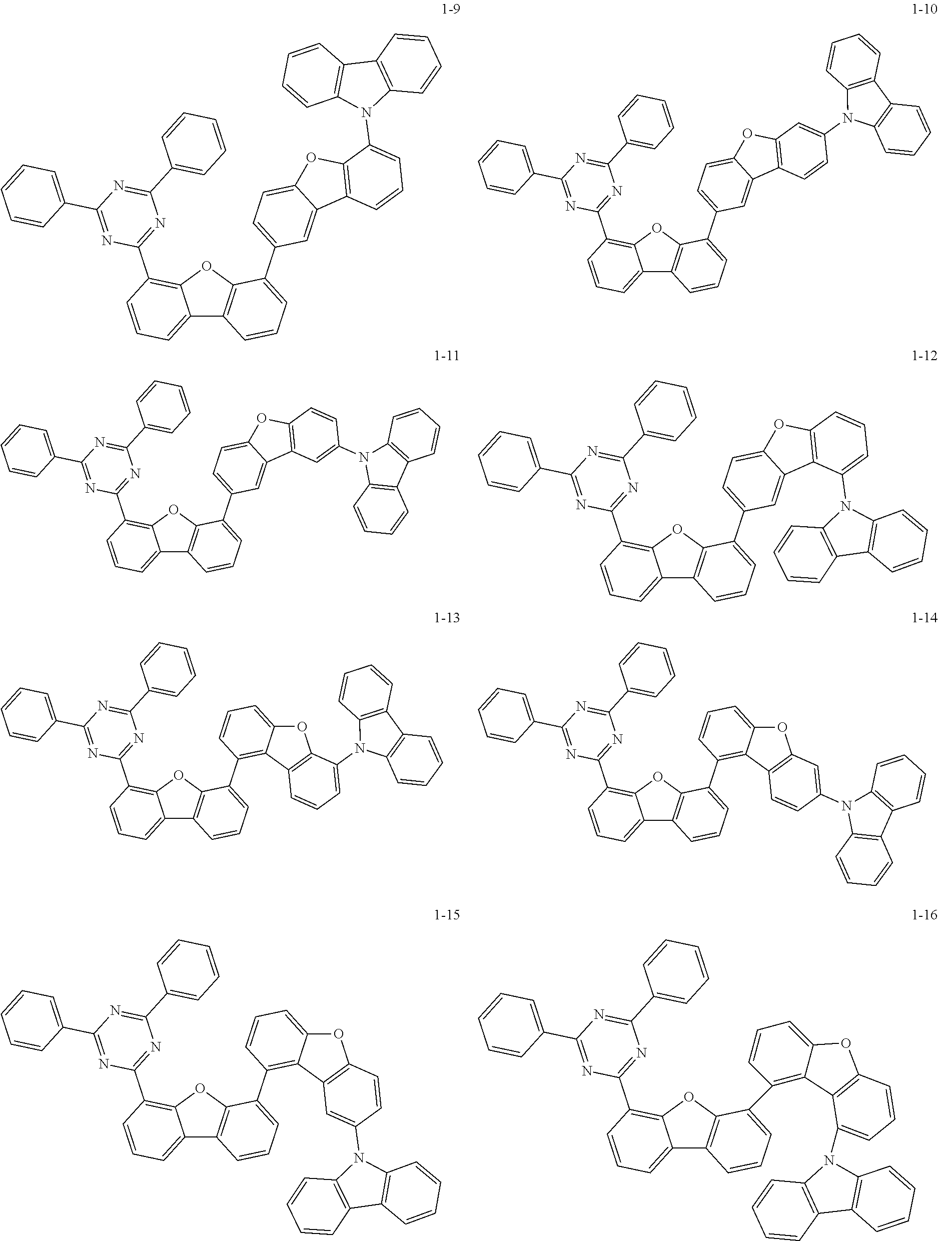

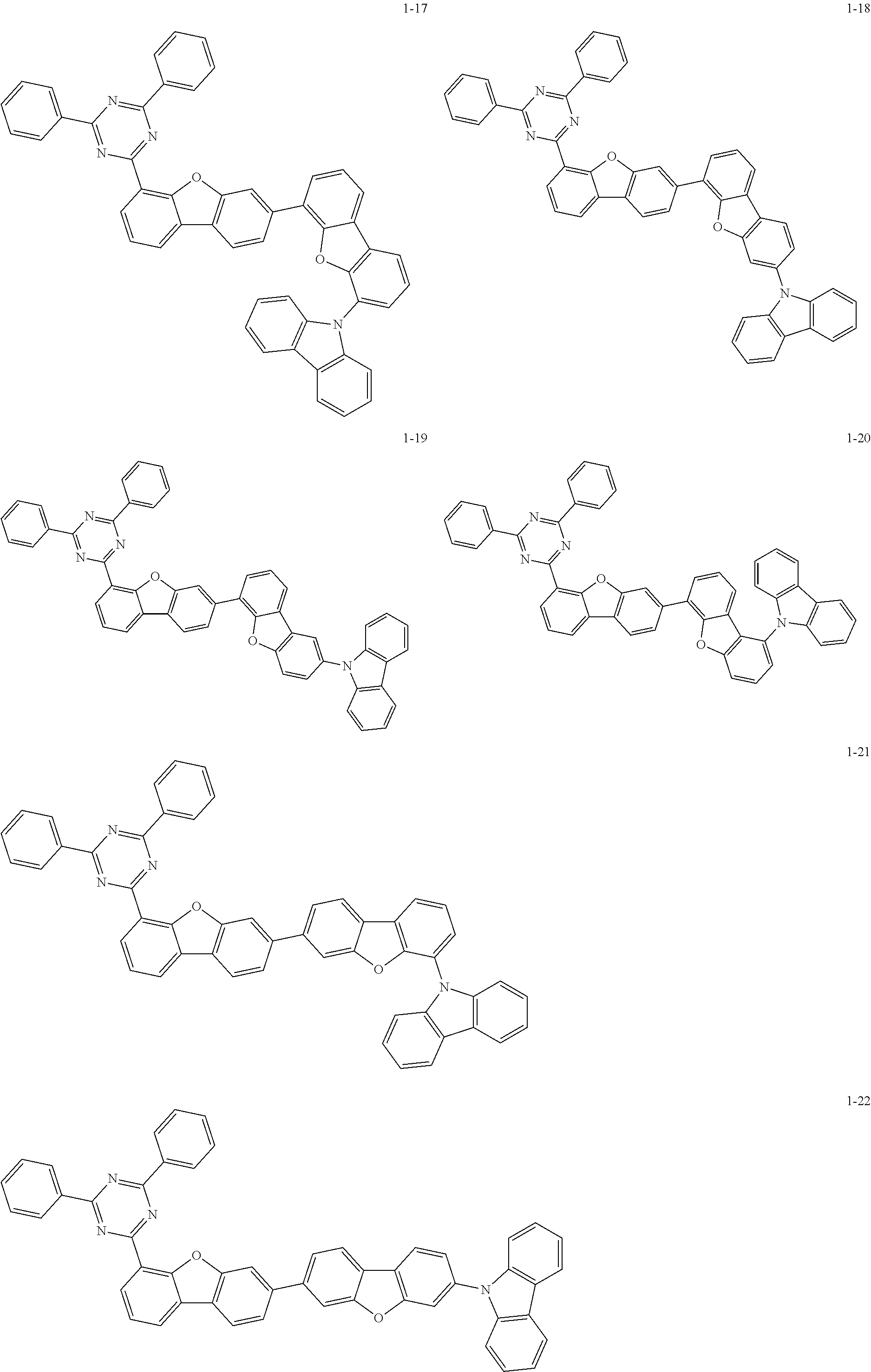

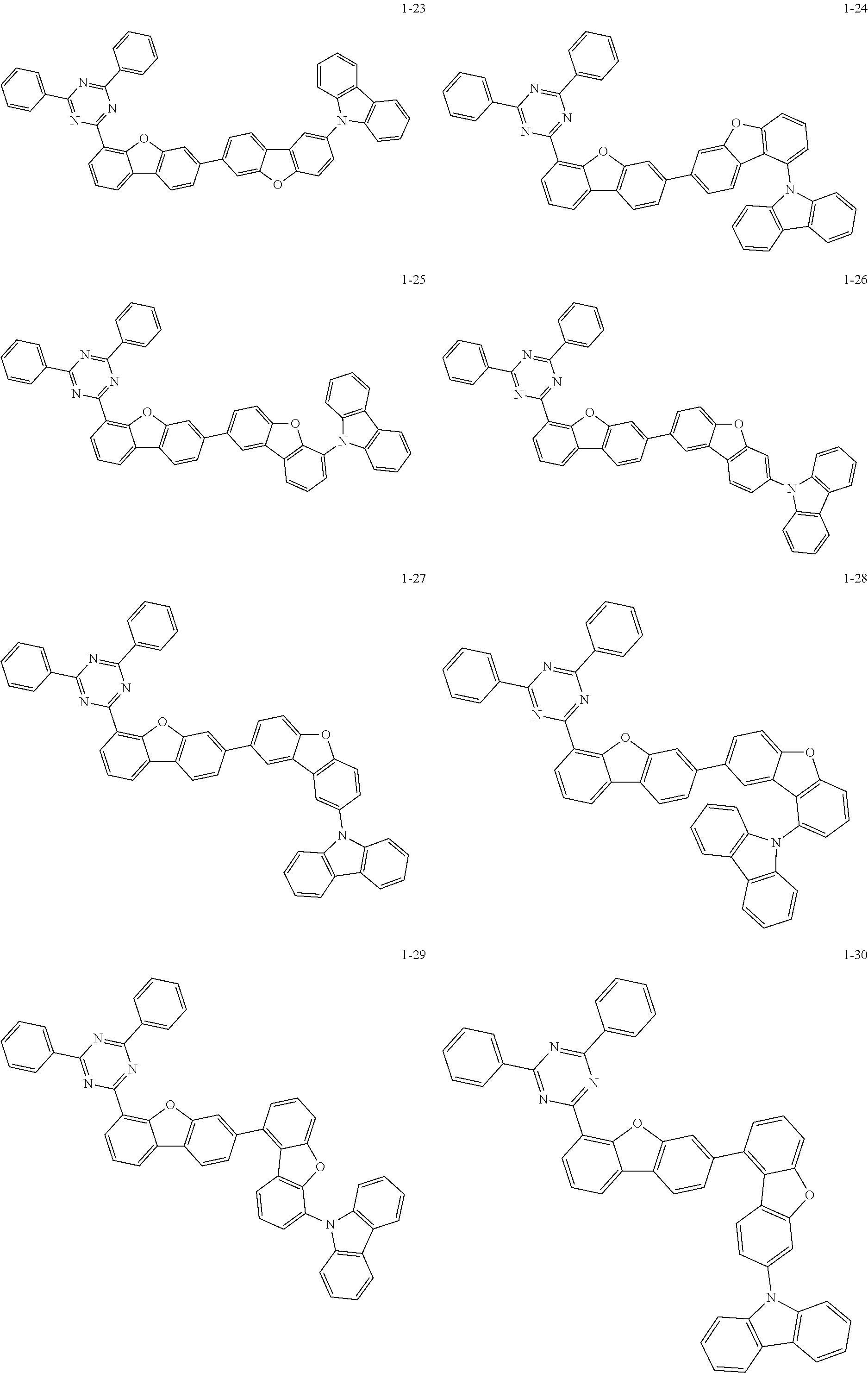

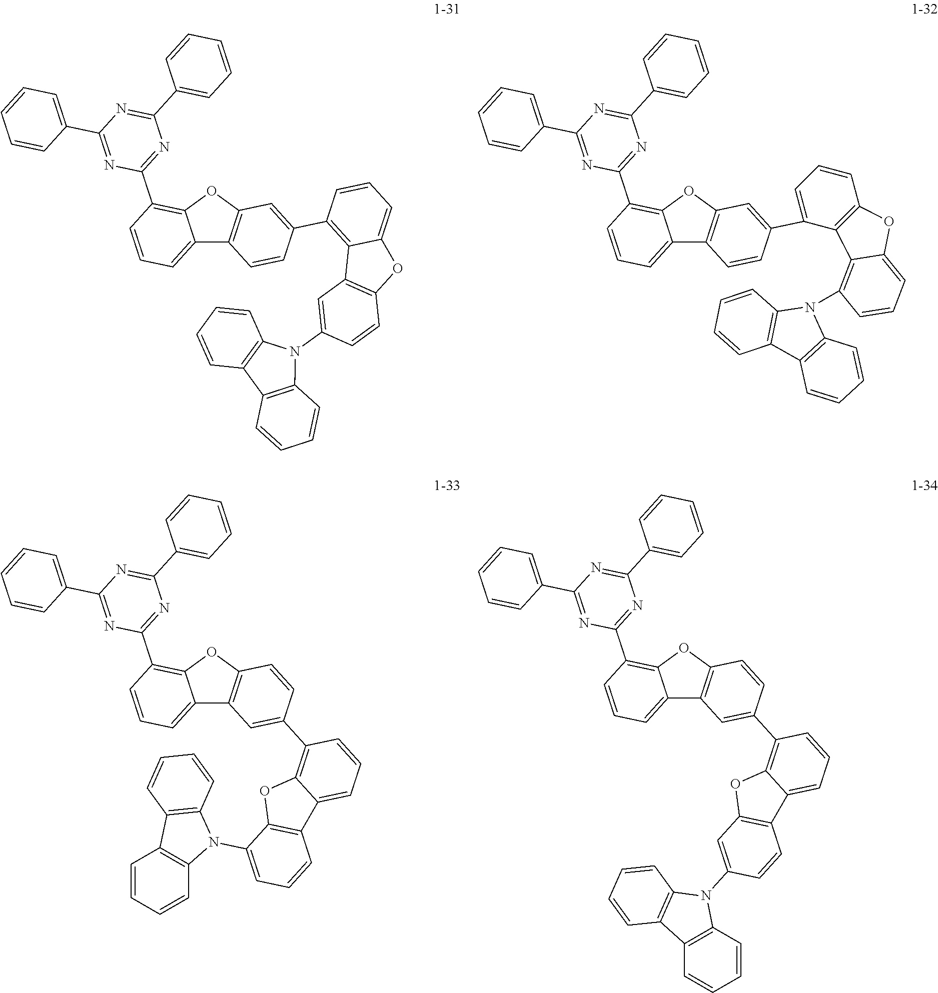 [SMILES: c1ccc(-c2nc(-c3ccccc3)nc(-c3cccc4c3oc3cc(-c5cccc6oc7ccc(-n8c9ccccc9c9ccccc98)cc7c56)ccc34)n2)cc1 ; c1ccc(-c2nc(-c3ccccc3)nc(-c3cccc4c3oc3cc(-c5cccc6oc7cccc(-n8c9ccccc9c9ccccc98)c7c56)ccc34)n2)cc1 ; c1ccc(-c2nc(-c3ccccc3)nc(-c3cccc4c3oc3ccc(-c5cccc6c5oc5c(-n7c8ccccc8c8ccccc87)cccc56)cc34)n2)cc1 ; c1ccc(-c2nc(-c3ccccc3)nc(-c3cccc4c3oc3ccc(-c5cccc6c5oc5cc(-n7c8ccccc8c8ccccc87)ccc56)cc34)n2)cc1]

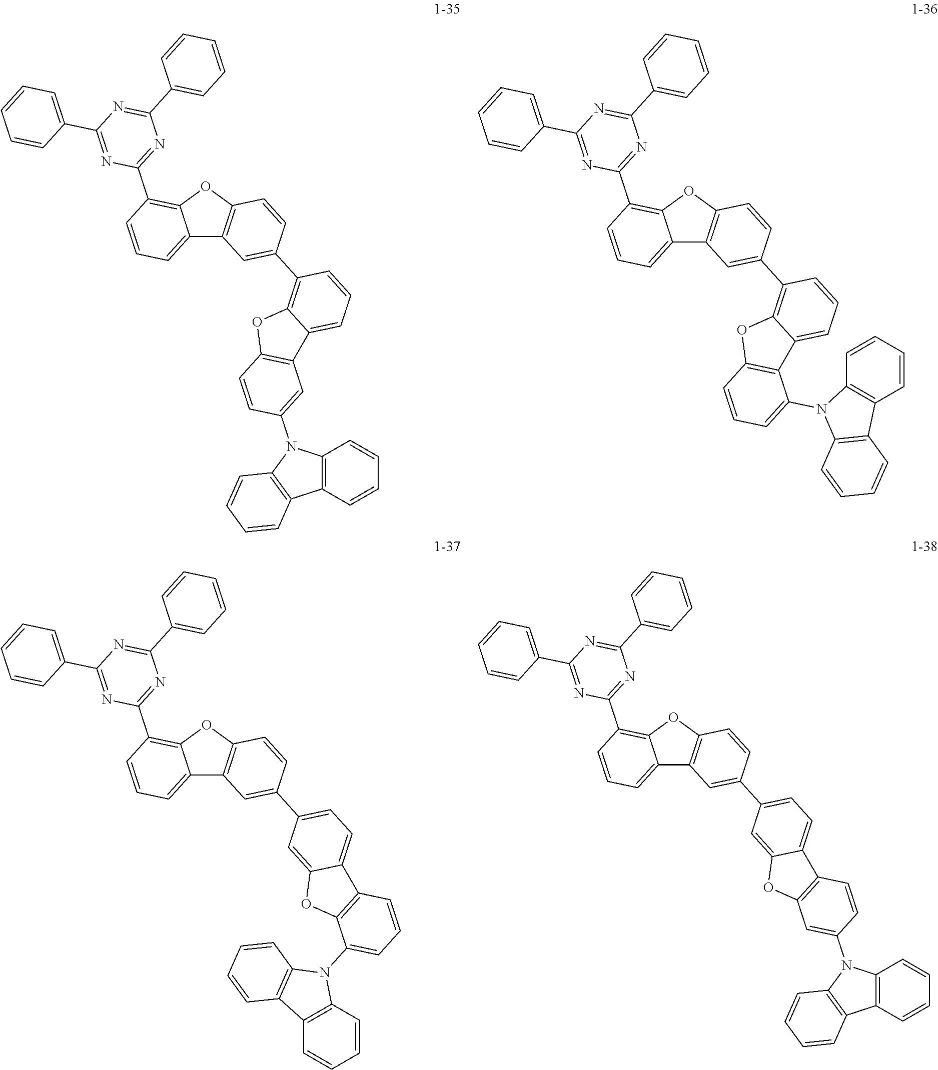 [SMILES: c1ccc(-c2nc(-c3ccccc3)nc(-c3cccc4c3oc3ccc(-c5cccc6c5oc5ccc(-n7c8ccccc8c8ccccc87)cc56)cc34)n2)cc1 ; c1ccc(-c2nc(-c3ccccc3)nc(-c3cccc4c3oc3ccc(-c5cccc6c5oc5cccc(-n7c8ccccc8c8ccccc87)c56)cc34)n2)cc1 ; c1ccc(-c2nc(-c3ccccc3)nc(-c3cccc4c3oc3ccc(-c5ccc6c(c5)oc5c(-n7c8ccccc8c8ccccc87)cccc56)cc34)n2)cc1 ; c1ccc(-c2nc(-c3ccccc3)nc(-c3cccc4c3oc3ccc(-c5ccc6c(c5)oc5cc(-n7c8ccccc8c8ccccc87)ccc56)cc34)n2)cc1]

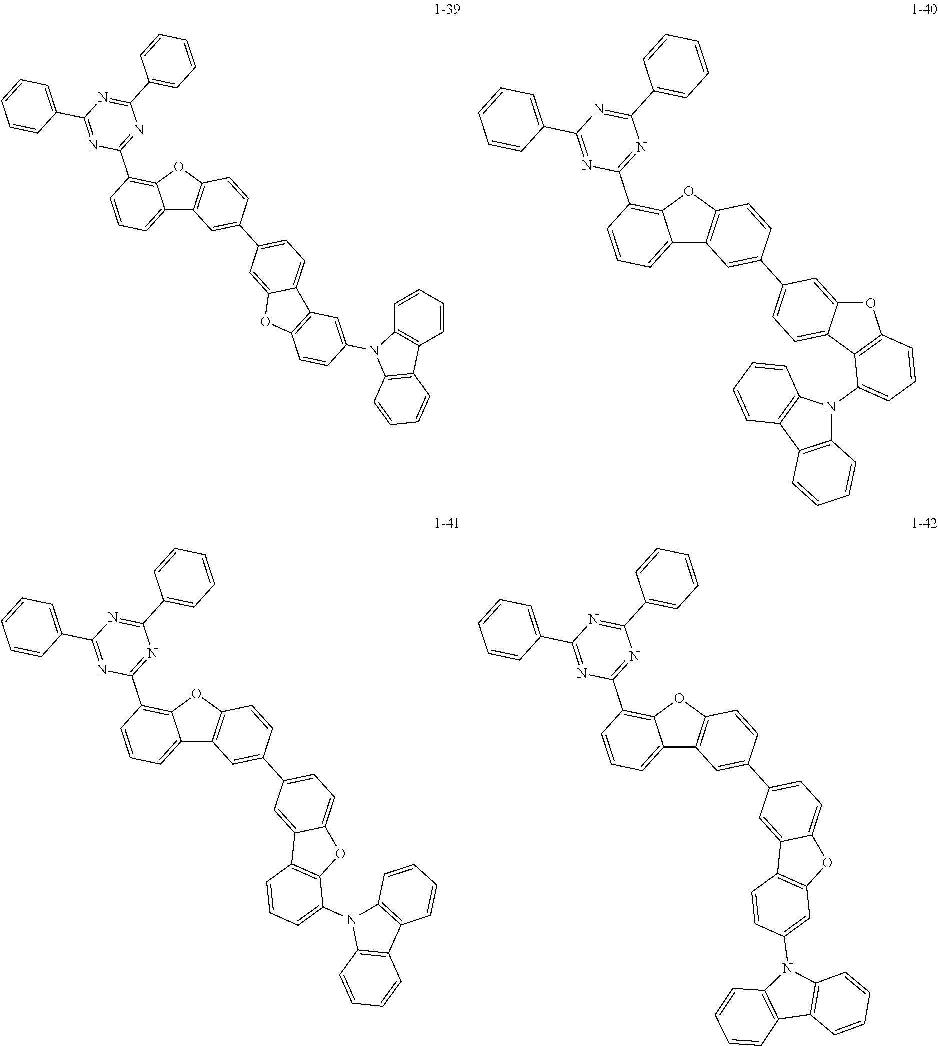 [SMILES: c1ccc(-c2nc(-c3ccccc3)nc(-c3cccc4c3oc3ccc(-c5ccc6c(c5)oc5ccc(-n7c8ccccc8c8ccccc87)cc56)cc34)n2)cc1 ; c1ccc(-c2nc(-c3ccccc3)nc(-c3cccc4c3oc3ccc(-c5ccc6c(c5)oc5cccc(-n7c8ccccc8c8ccccc87)c56)cc34)n2)cc1 ; c1ccc(-c2nc(-c3ccccc3)nc(-c3cccc4c3oc3ccc(-c5ccc6oc7c(-n8c9ccccc9c9ccccc98)cccc7c6c5)cc34)n2)cc1 ; c1ccc(-c2nc(-c3ccccc3)nc(-c3cccc4c3oc3ccc(-c5ccc6oc7cc(-n8c9ccccc9c9ccccc98)ccc7c6c5)cc34)n2)cc1]

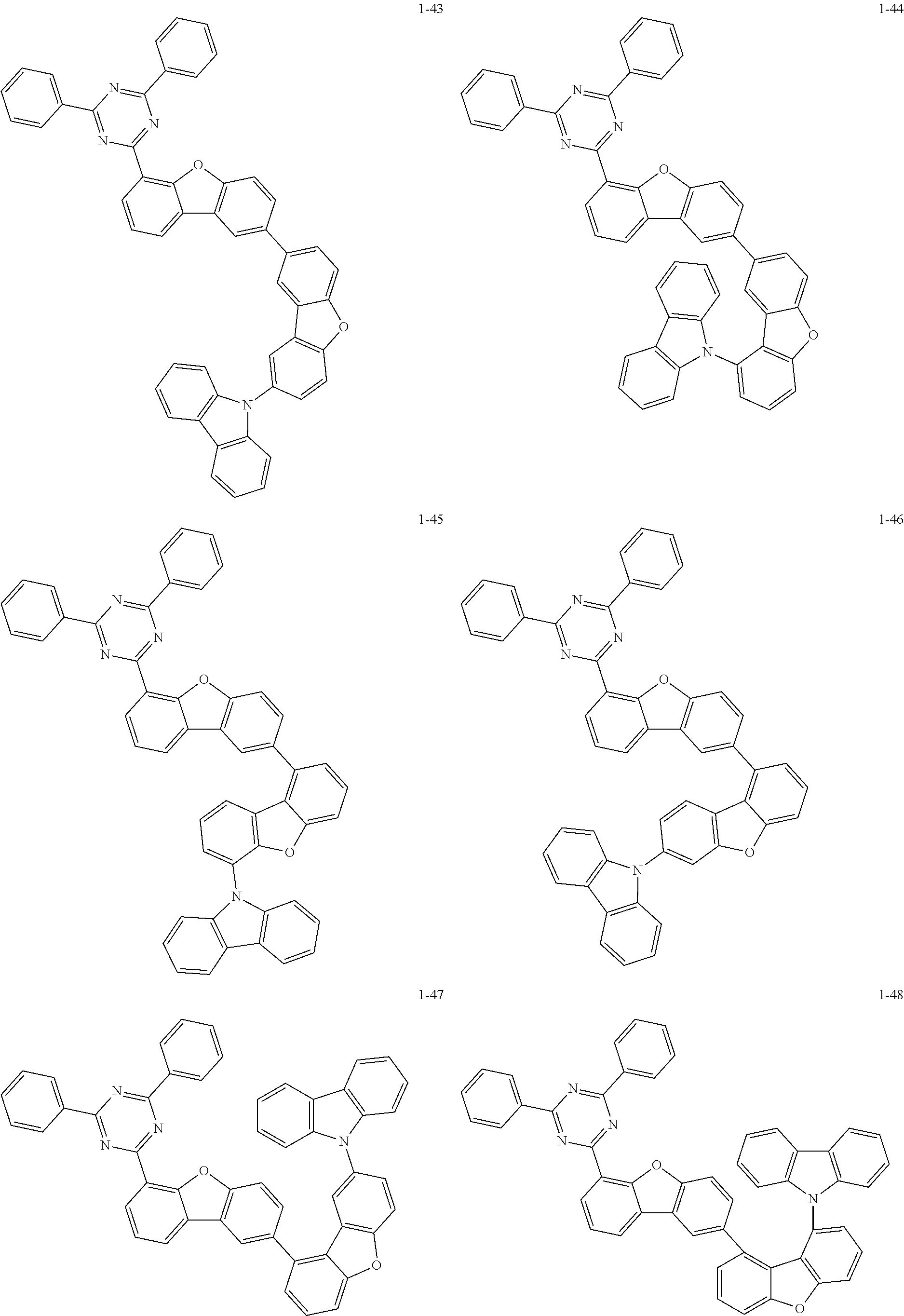

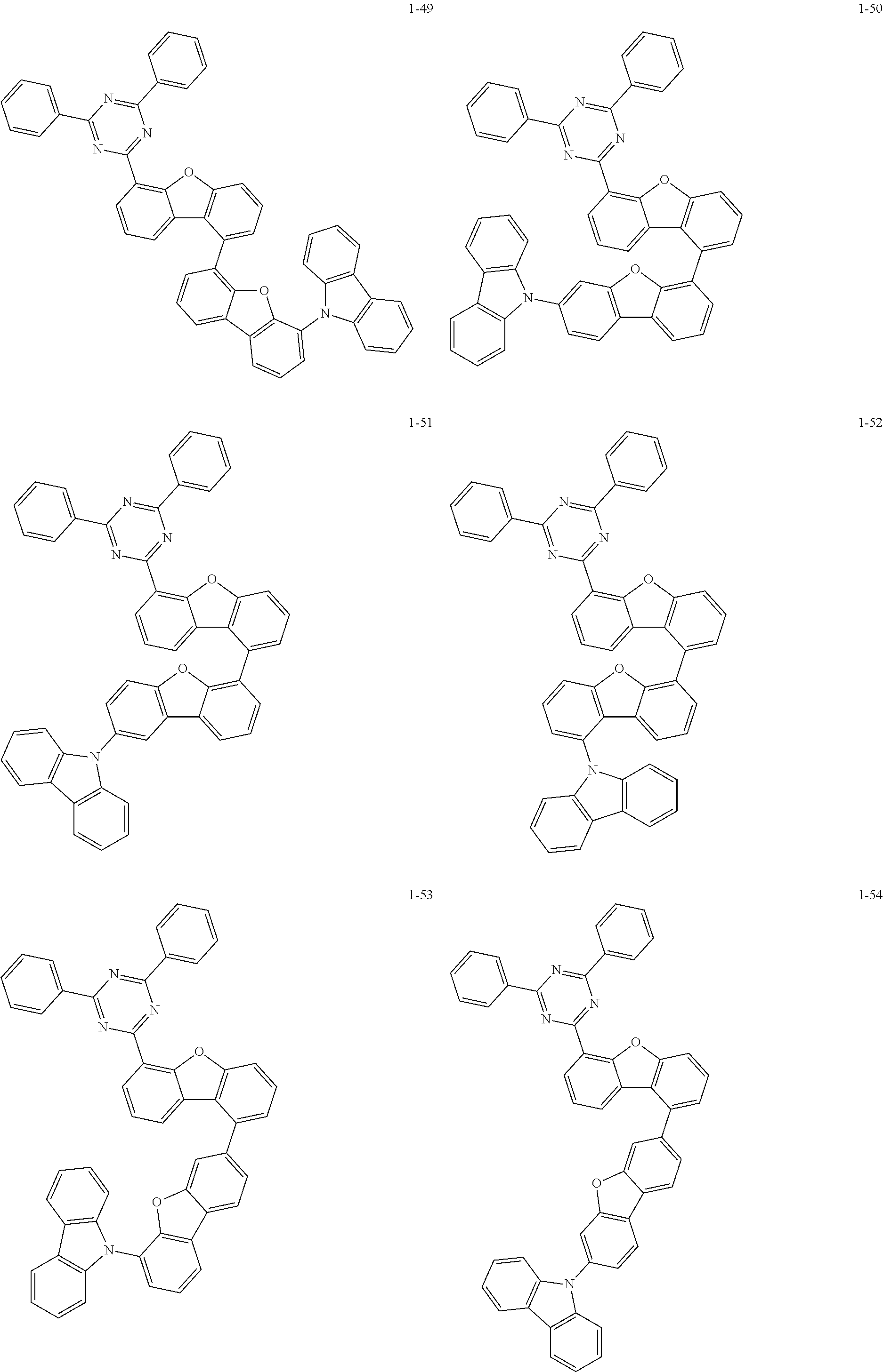

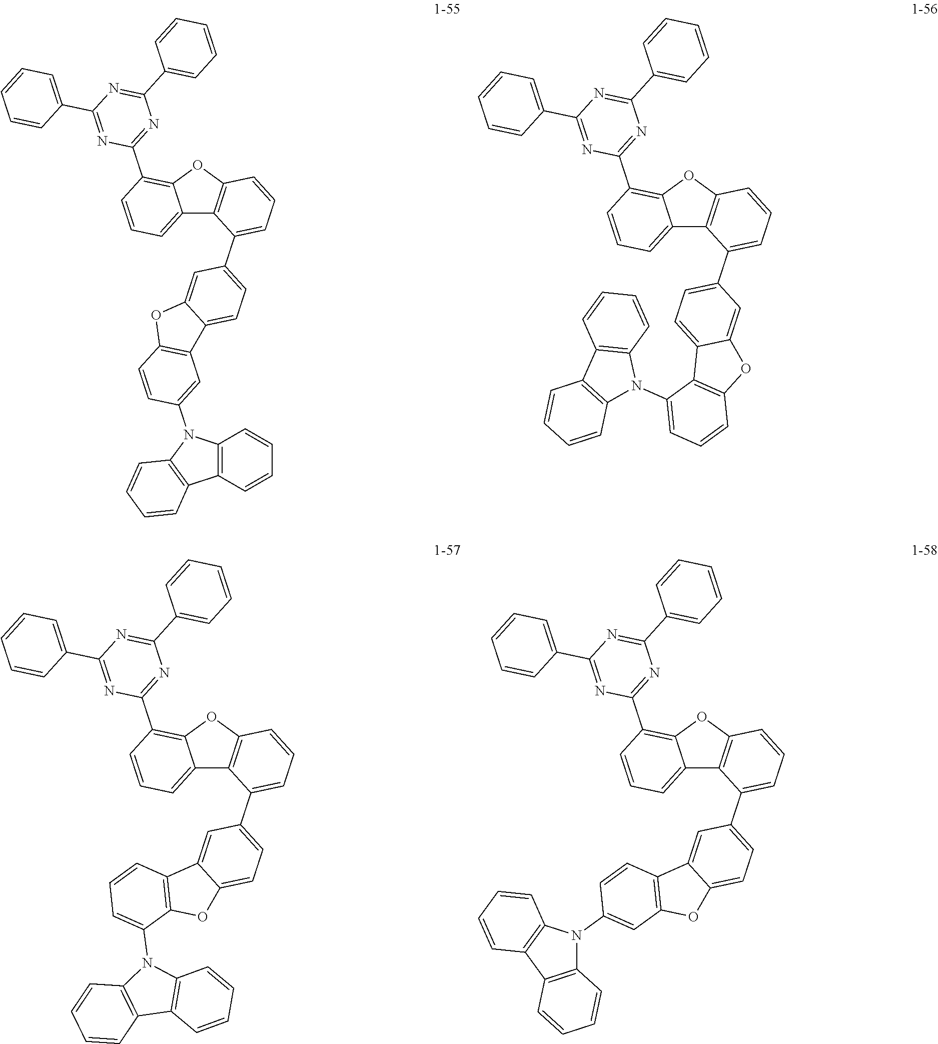 [SMILES: c1ccc(-c2nc(-c3ccccc3)nc(-c3cccc4c3oc3cccc(-c5ccc6c(c5)oc5ccc(-n7c8ccccc8c8ccccc87)cc56)c34)n2)cc1 ; c1ccc(-c2nc(-c3ccccc3)nc(-c3cccc4c3oc3cccc(-c5ccc6c(c5)oc5cccc(-n7c8ccccc8c8ccccc87)c56)c34)n2)cc1 ; c1ccc(-c2nc(-c3ccccc3)nc(-c3cccc4c3oc3cccc(-c5ccc6oc7c(-n8c9ccccc9c9ccccc98)cccc7c6c5)c34)n2)cc1 ; c1ccc(-c2nc(-c3ccccc3)nc(-c3cccc4c3oc3cccc(-c5ccc6oc7cc(-n8c9ccccc9c9ccccc98)ccc7c6c5)c34)n2)cc1]

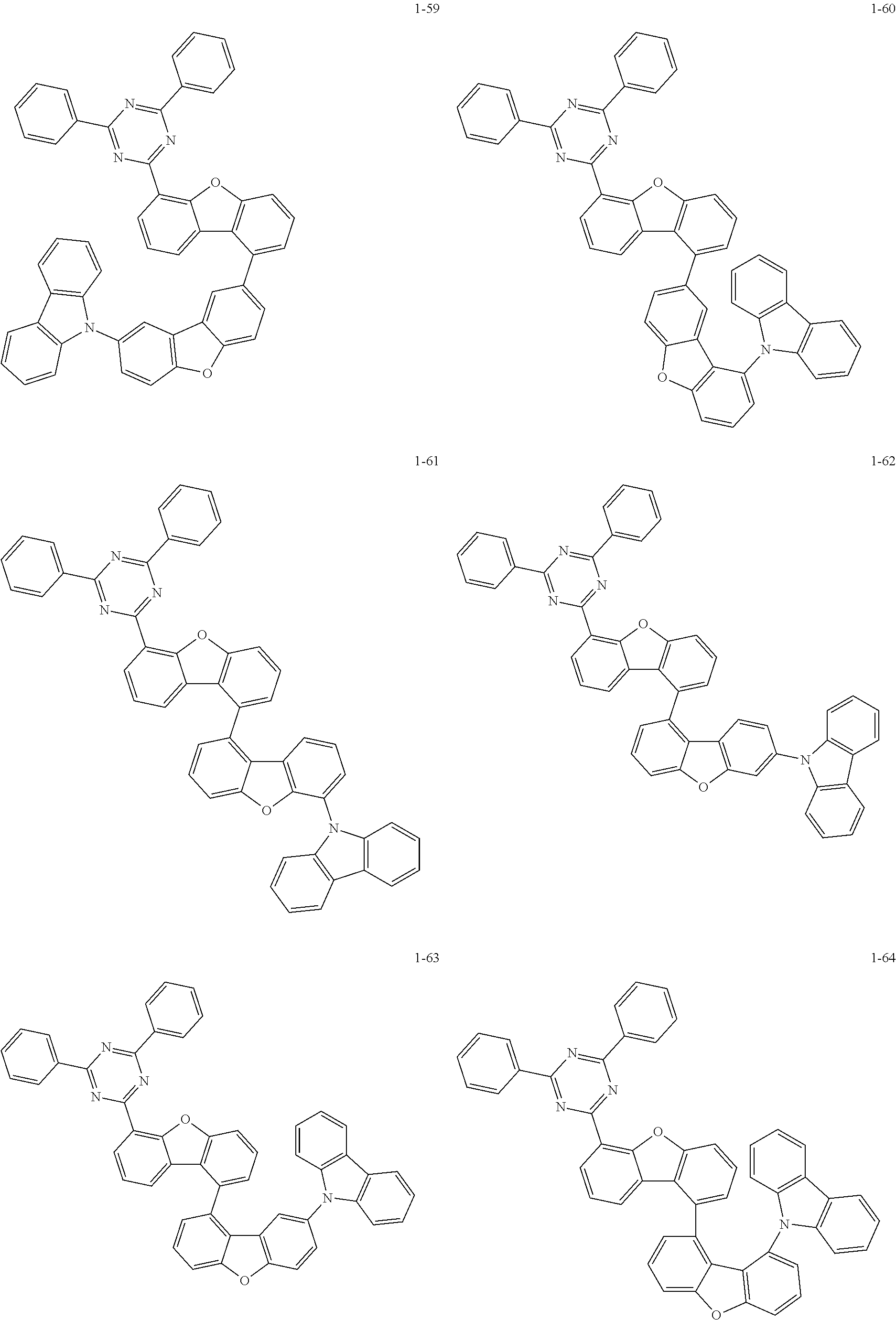

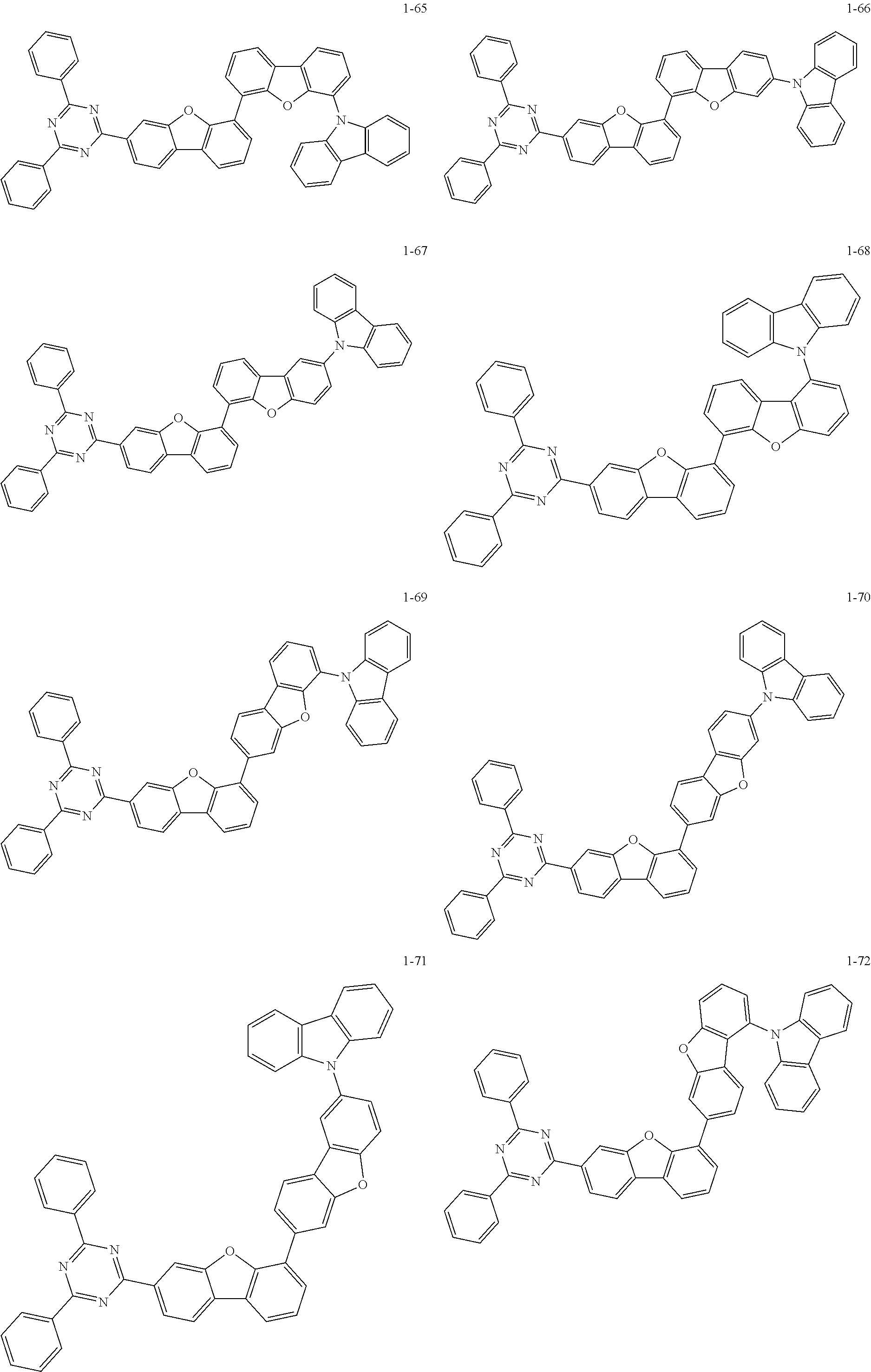

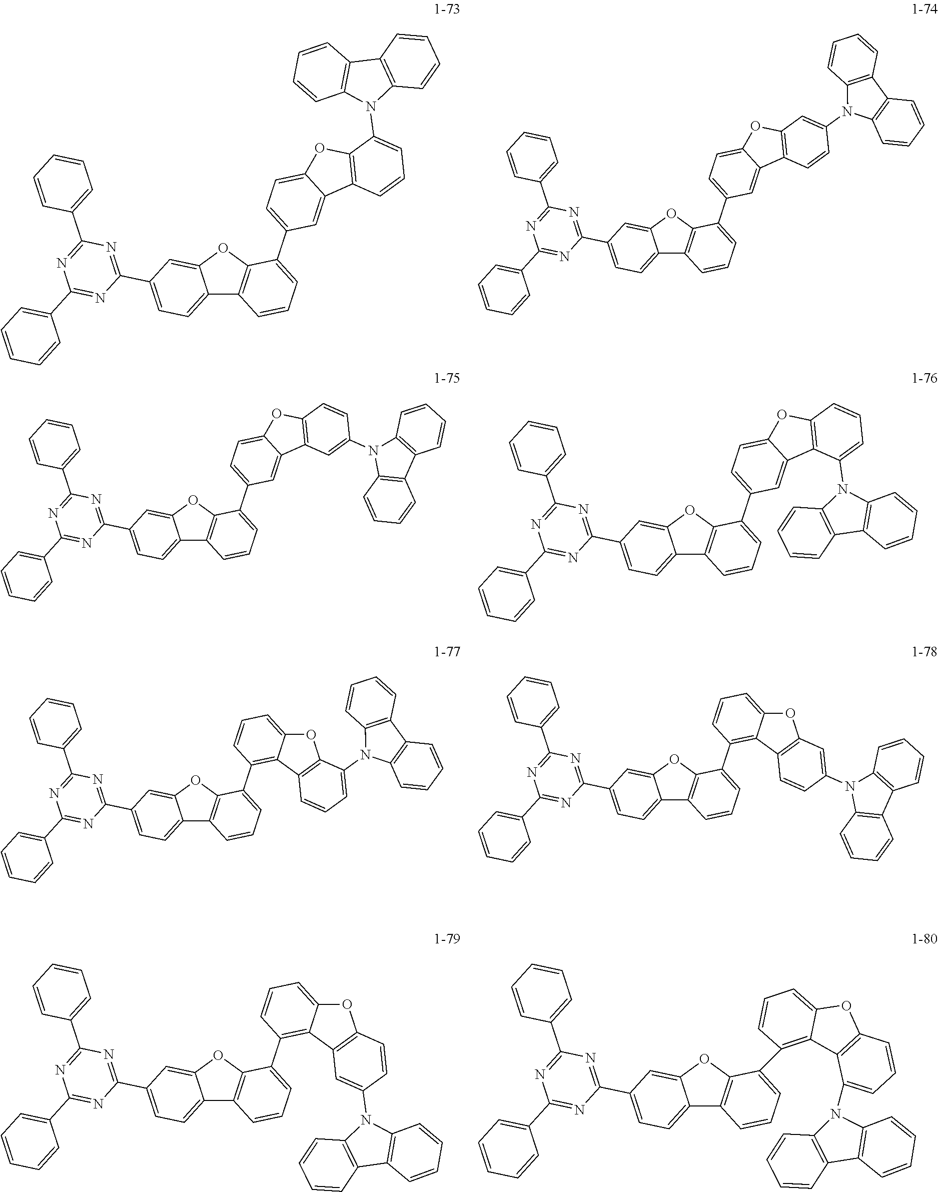

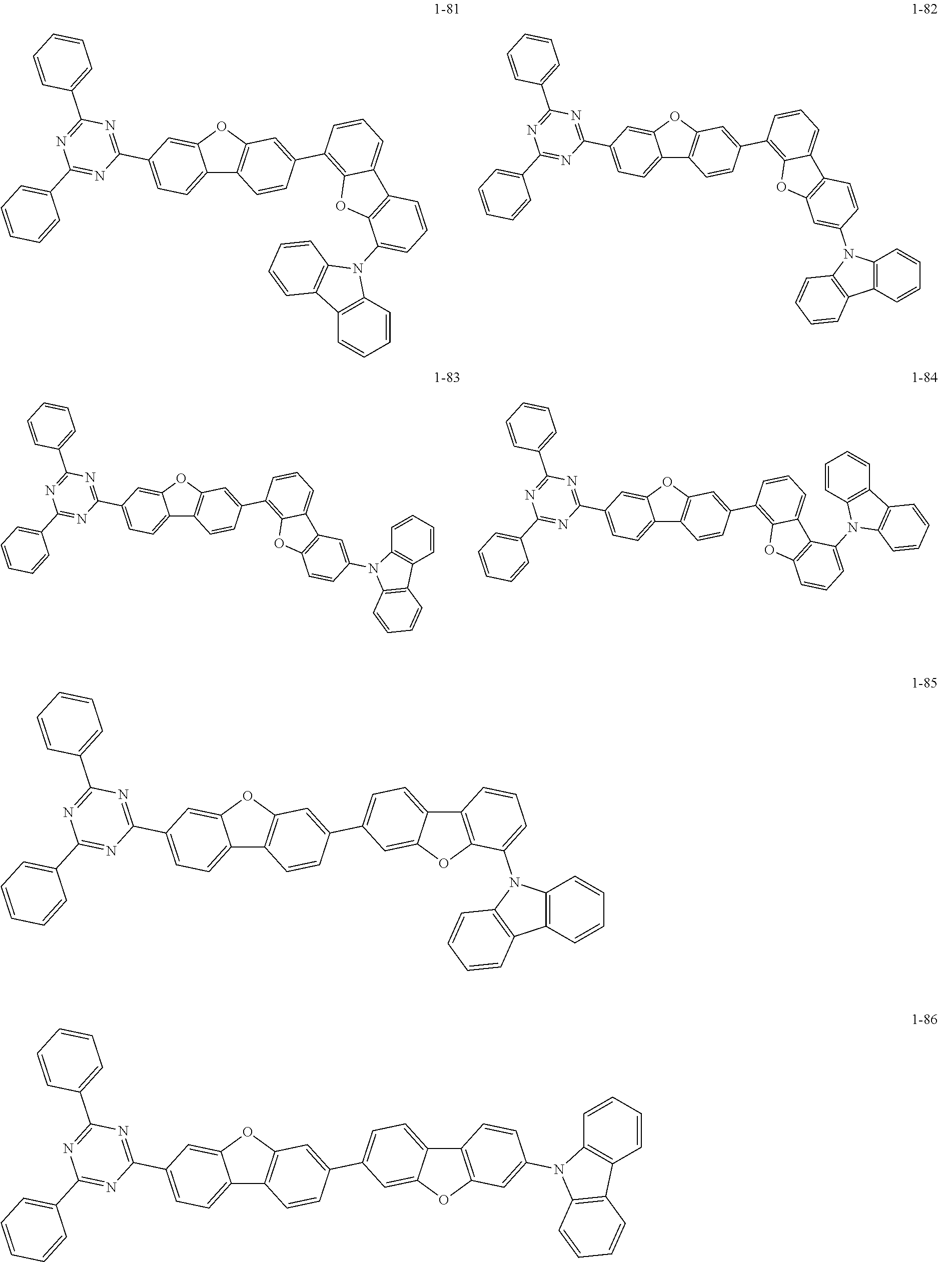

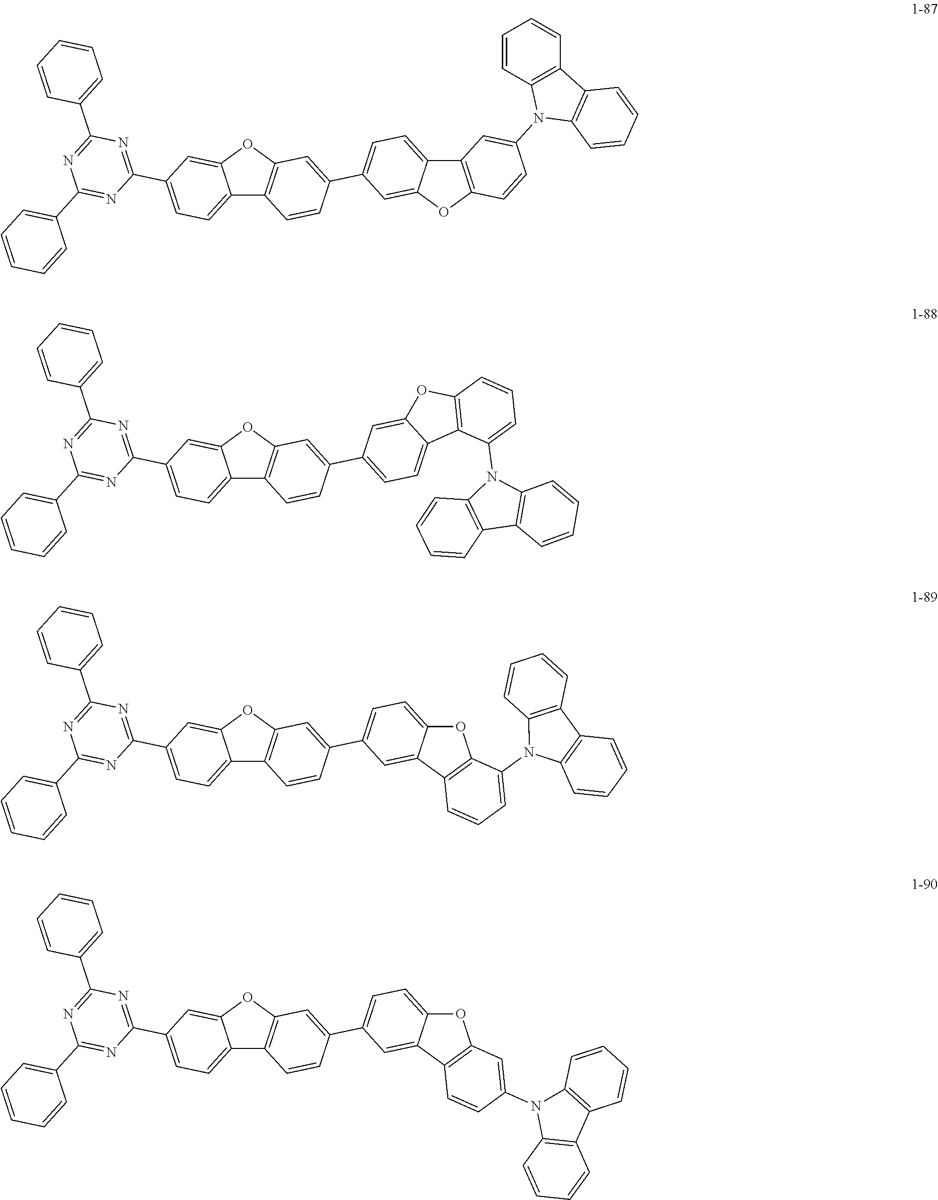 [SMILES: c1ccc(-c2nc(-c3ccccc3)nc(-c3ccc4c(c3)oc3cc(-c5ccc6c(c5)oc5ccc(-n7c8ccccc8c8ccccc87)cc56)ccc34)n2)cc1 ; c1ccc(-c2nc(-c3ccccc3)nc(-c3ccc4c(c3)oc3cc(-c5ccc6c(c5)oc5cccc(-n7c8ccccc8c8ccccc87)c56)ccc34)n2)cc1 ; c1ccc(-c2nc(-c3ccccc3)nc(-c3ccc4c(c3)oc3cc(-c5ccc6oc7c(-n8c9ccccc9c9ccccc98)cccc7c6c5)ccc34)n2)cc1 ; c1ccc(-c2nc(-c3ccccc3)nc(-c3ccc4c(c3)oc3cc(-c5ccc6oc7cc(-n8c9ccccc9c9ccccc98)ccc7c6c5)ccc34)n2)cc1]

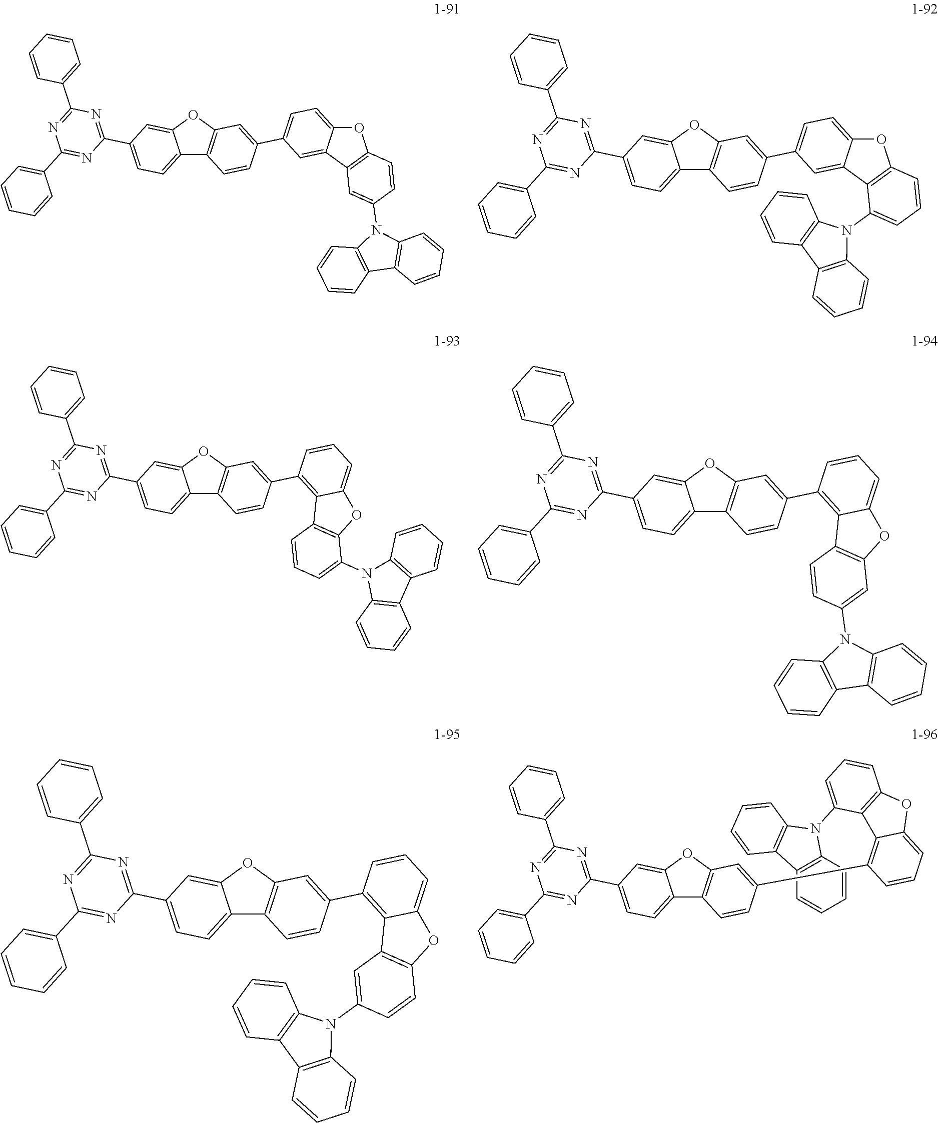

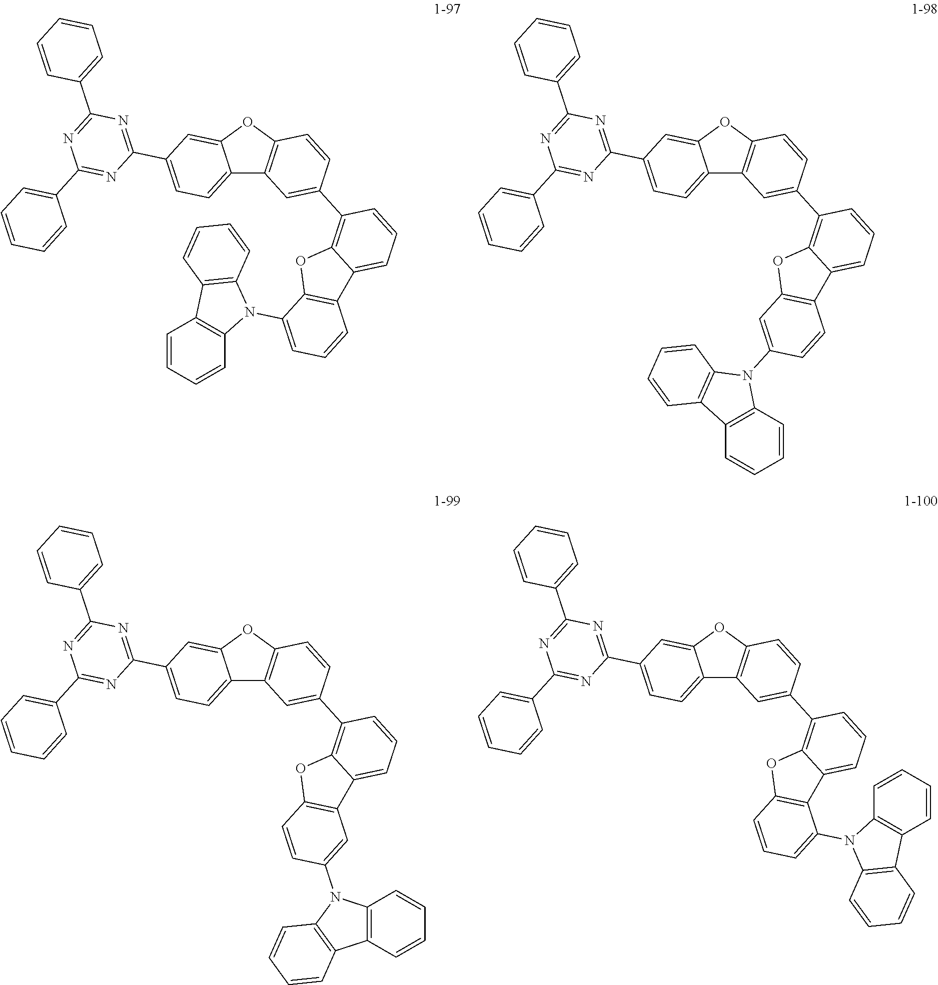 [SMILES: c1ccc(-c2nc(-c3ccccc3)nc(-c3ccc4c(c3)oc3ccc(-c5cccc6c5oc5c(-n7c8ccccc8c8ccccc87)cccc56)cc34)n2)cc1 ; c1ccc(-c2nc(-c3ccccc3)nc(-c3ccc4c(c3)oc3ccc(-c5cccc6c5oc5cc(-n7c8ccccc8c8ccccc87)ccc56)cc34)n2)cc1 ; c1ccc(-c2nc(-c3ccccc3)nc(-c3ccc4c(c3)oc3ccc(-c5cccc6c5oc5ccc(-n7c8ccccc8c8ccccc87)cc56)cc34)n2)cc1 ; c1ccc(-c2nc(-c3ccccc3)nc(-c3ccc4c(c3)oc3ccc(-c5cccc6c5oc5cccc(-n7c8ccccc8c8ccccc87)c56)cc34)n2)cc1]

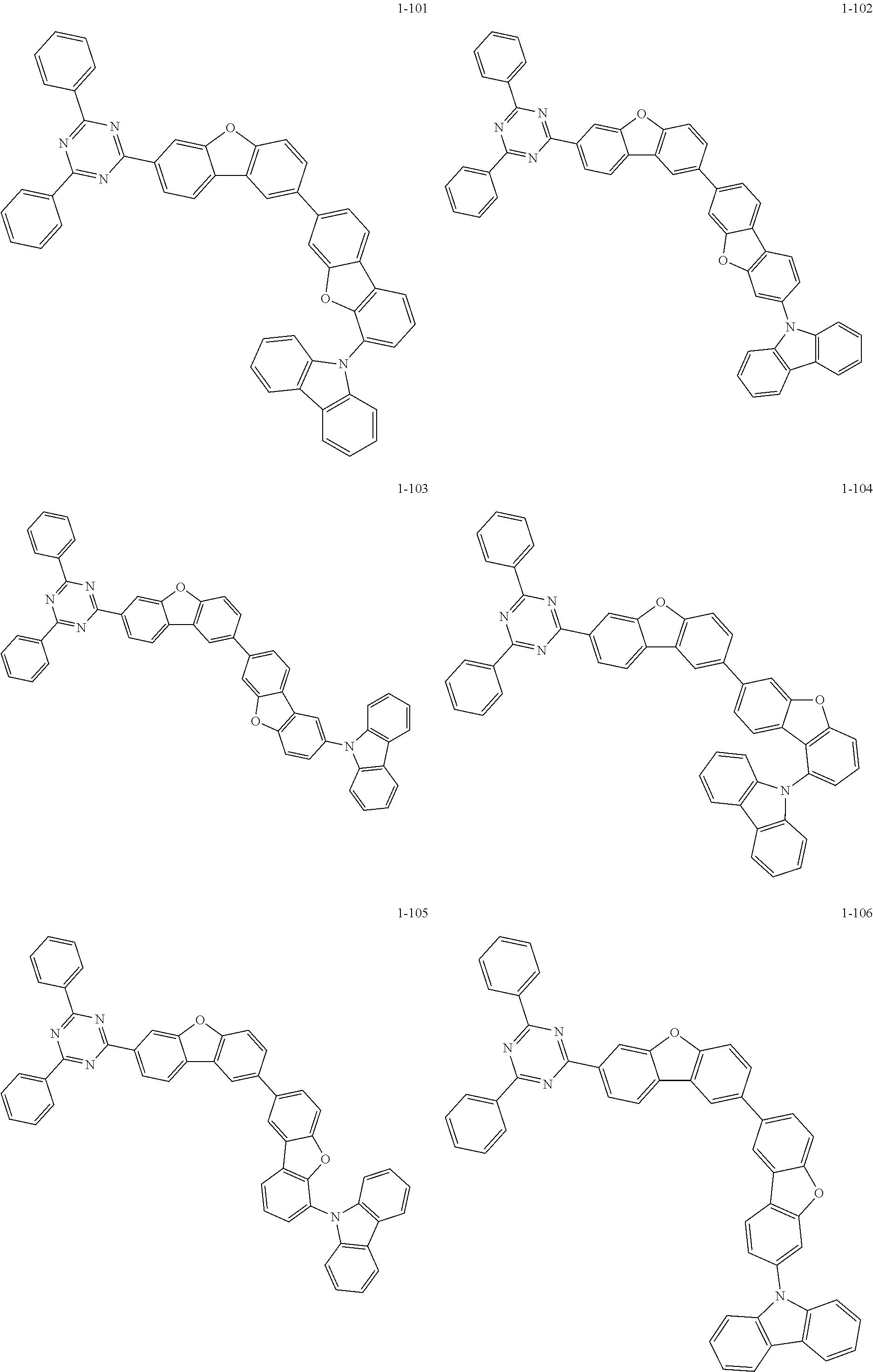

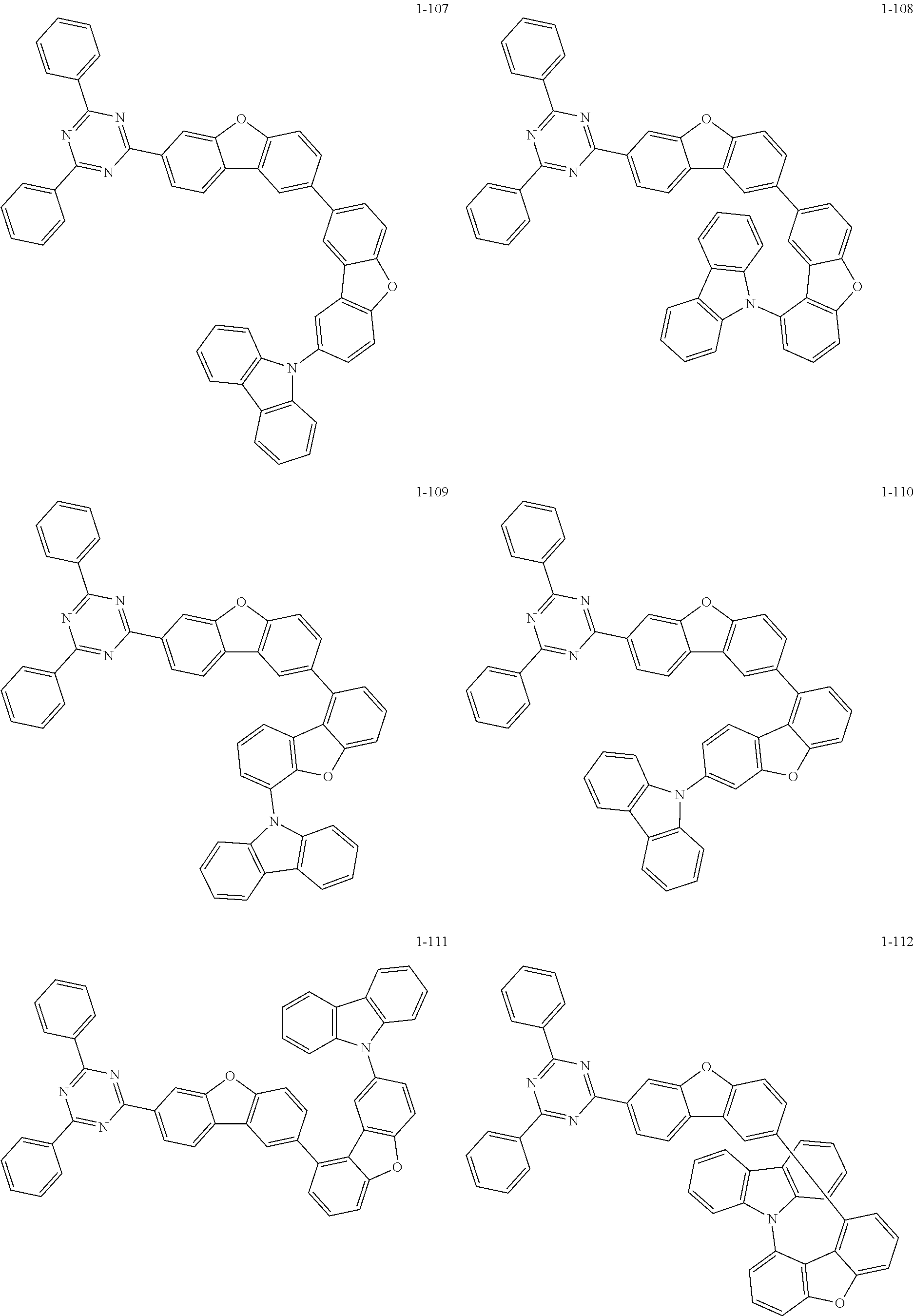

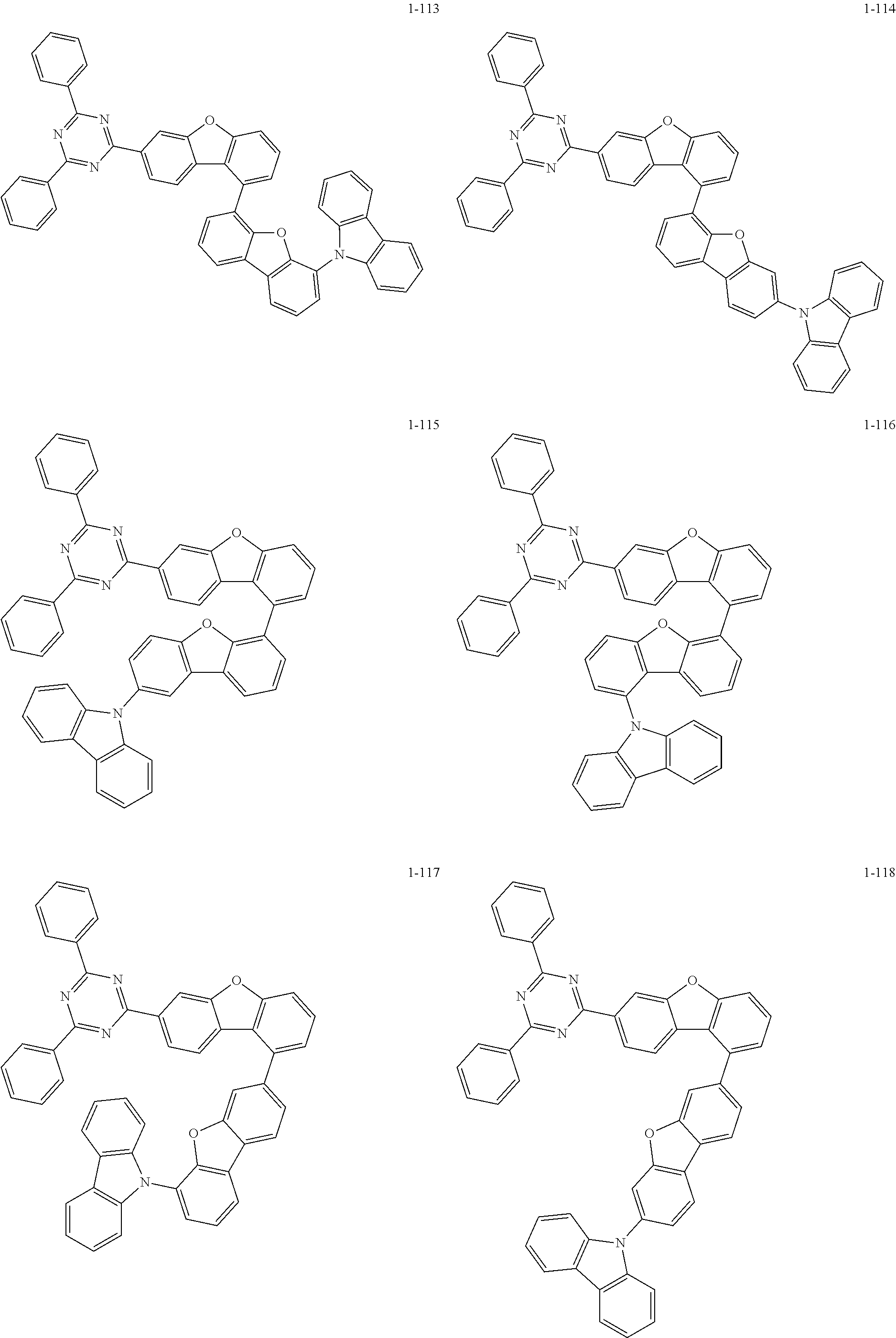

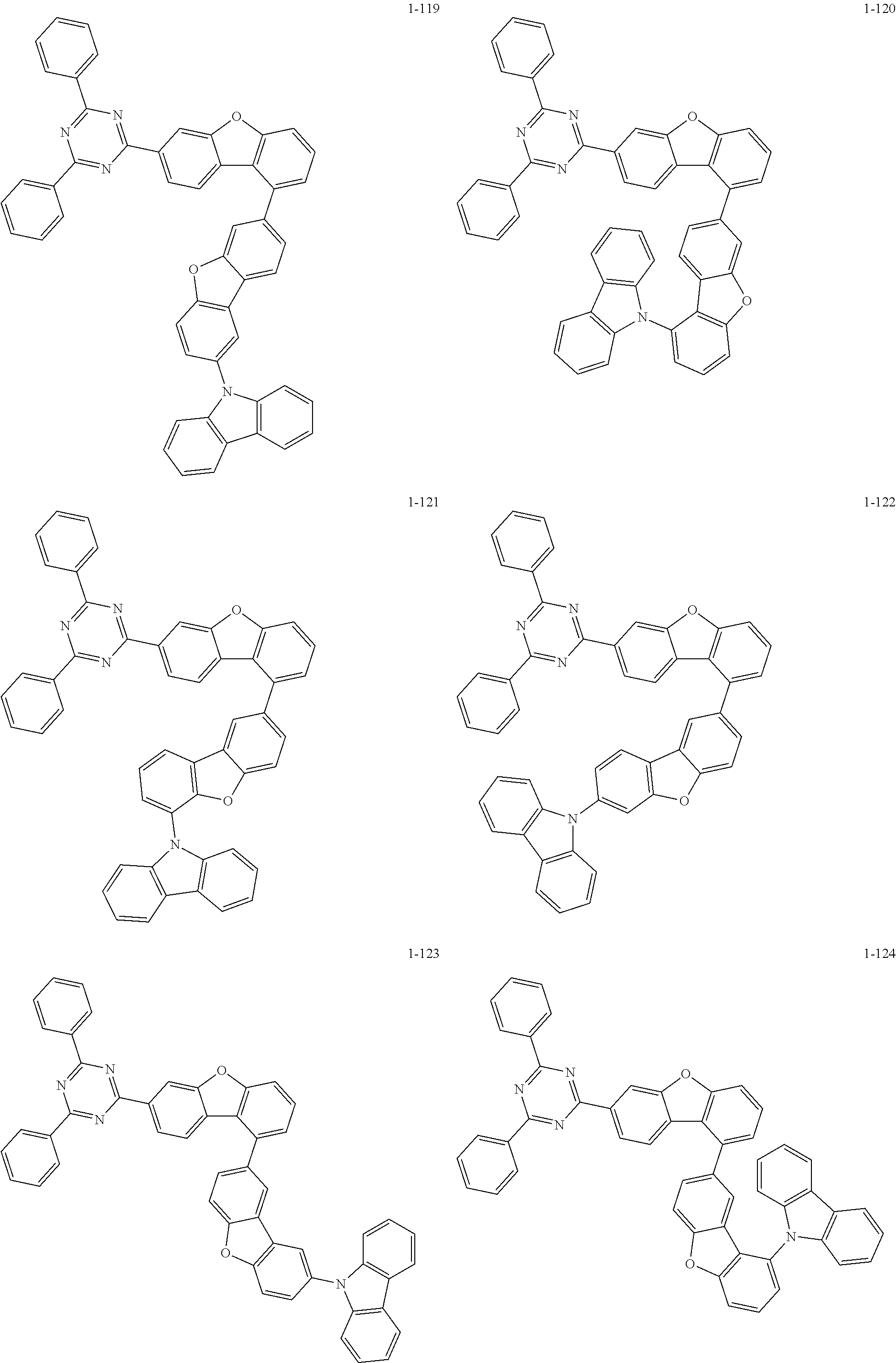

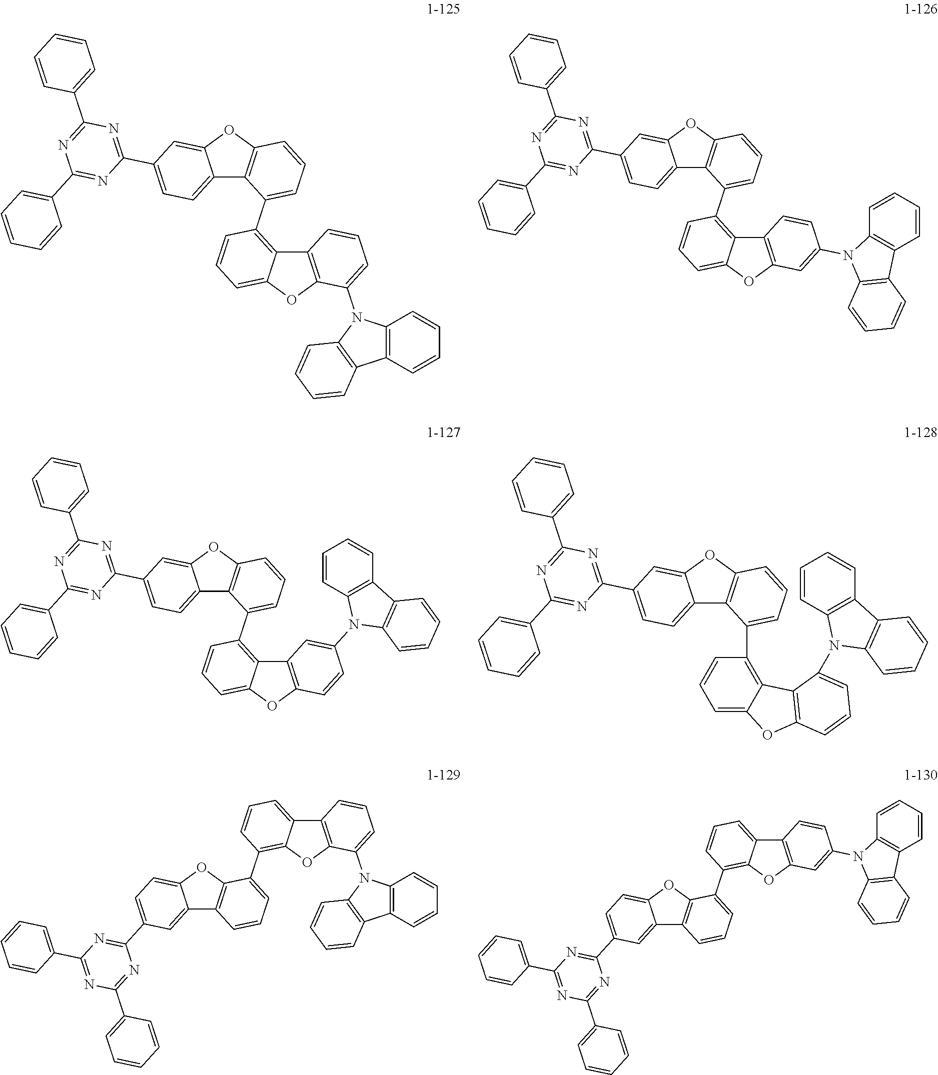

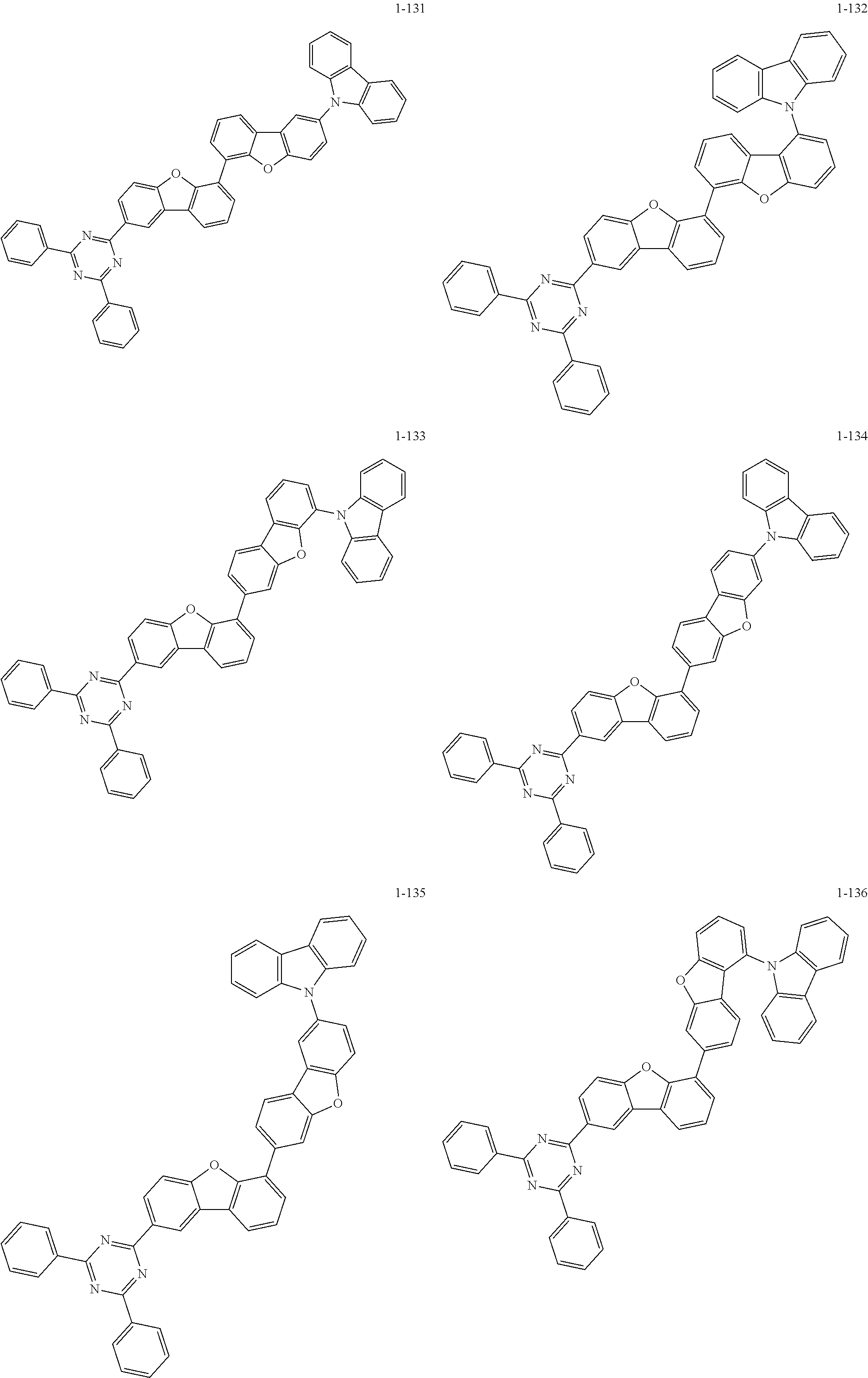

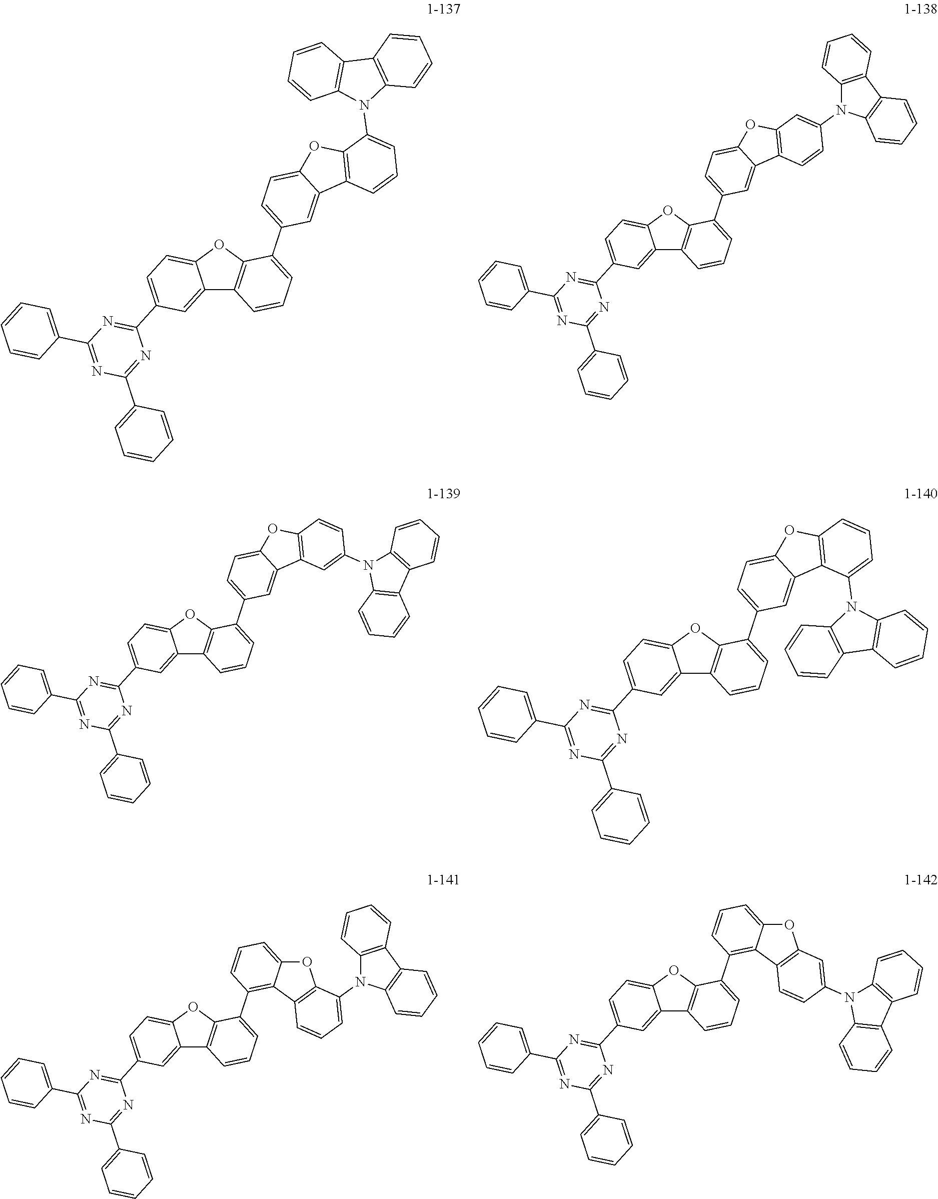

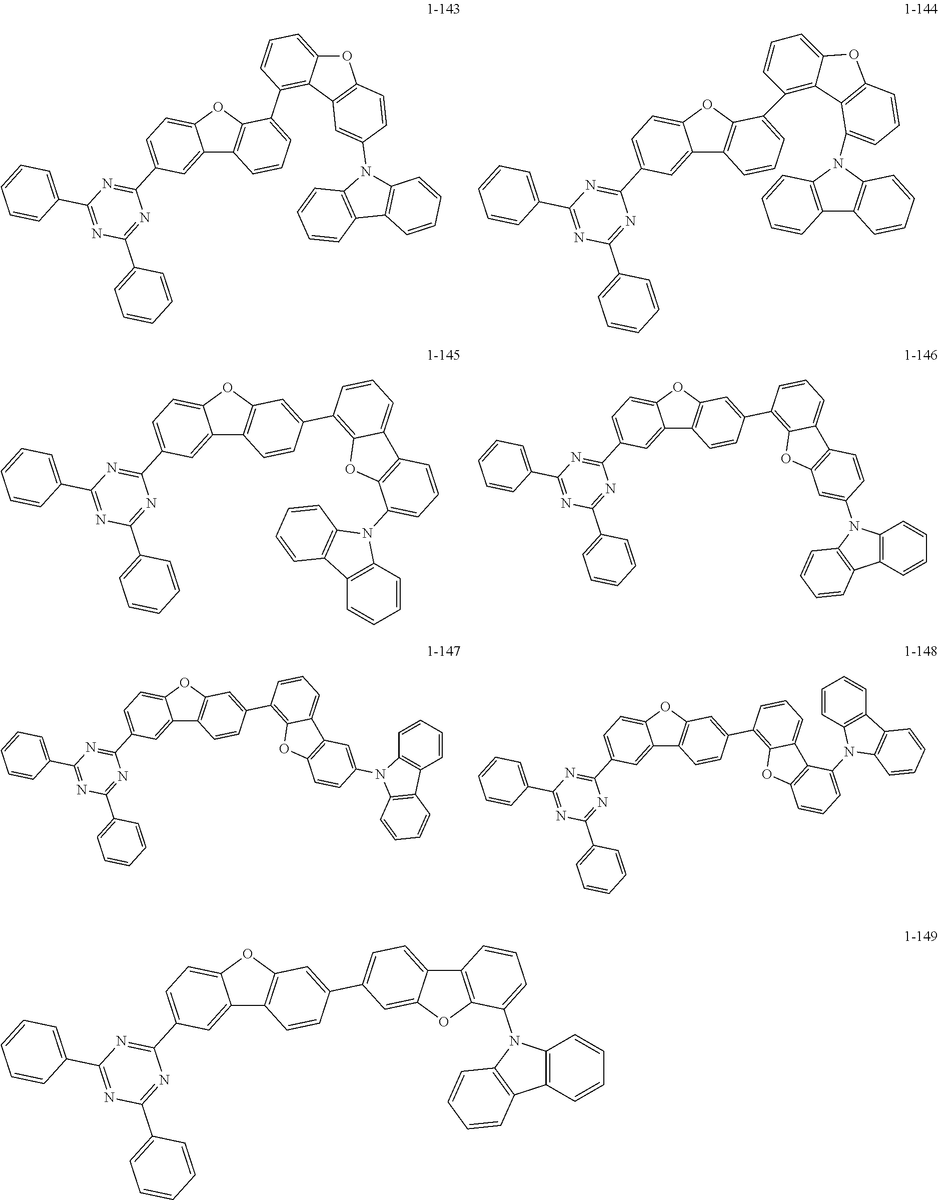

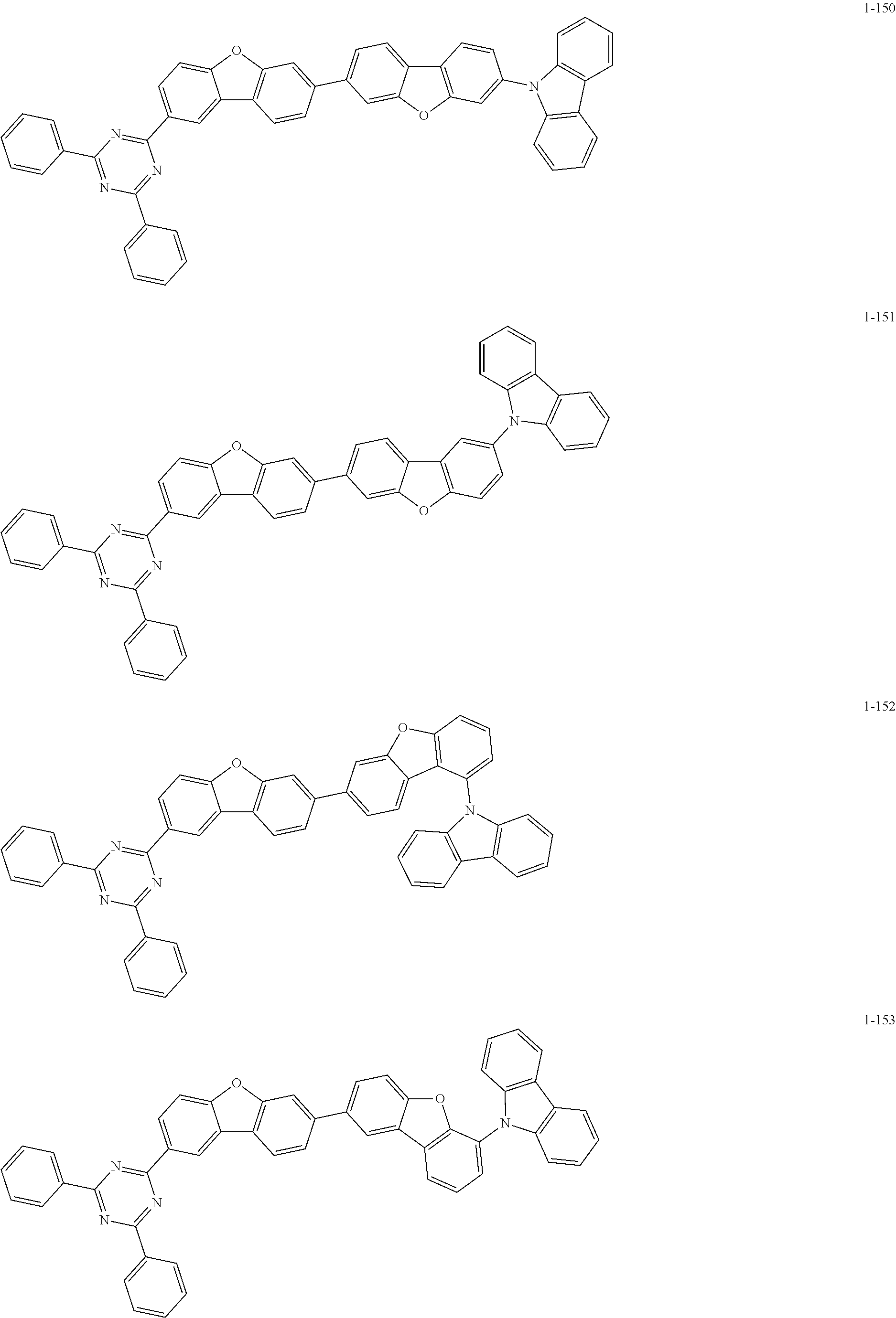 [SMILES: c1ccc(-c2nc(-c3ccccc3)nc(-c3ccc4oc5cc(-c6ccc7c(c6)oc6cc(-n8c9ccccc9c9ccccc98)ccc67)ccc5c4c3)n2)cc1 ; c1ccc(-c2nc(-c3ccccc3)nc(-c3ccc4oc5cc(-c6ccc7c(c6)oc6ccc(-n8c9ccccc9c9ccccc98)cc67)ccc5c4c3)n2)cc1 ; c1ccc(-c2nc(-c3ccccc3)nc(-c3ccc4oc5cc(-c6ccc7c(c6)oc6cccc(-n8c9ccccc9c9ccccc98)c67)ccc5c4c3)n2)cc1 ; c1ccc(-c2nc(-c3ccccc3)nc(-c3ccc4oc5cc(-c6ccc7oc8c(-n9c%10ccccc%10c%10ccccc%109)cccc8c7c6)ccc5c4c3)n2)cc1]

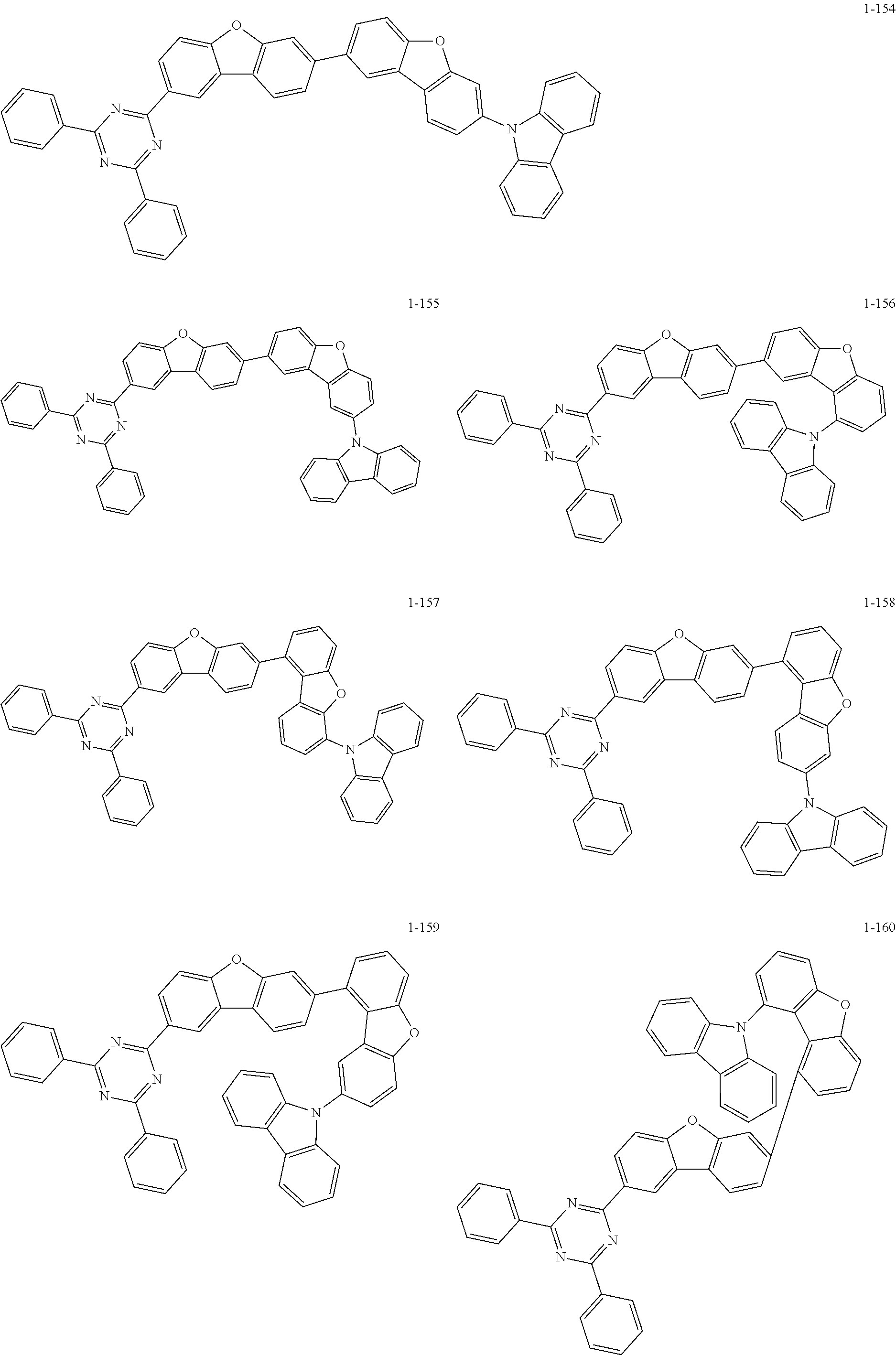

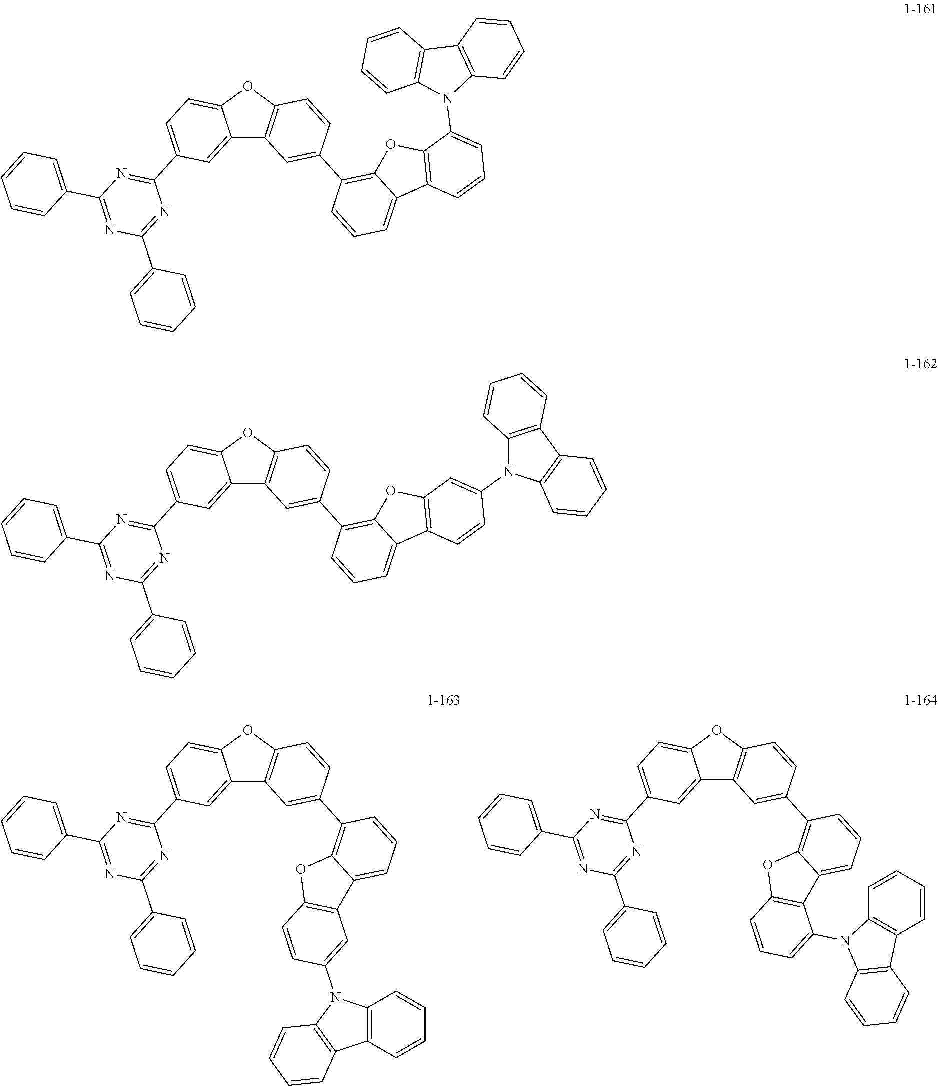 [SMILES: c1ccc(-c2nc(-c3ccccc3)nc(-c3ccc4oc5ccc(-c6cccc7c6oc6c(-n8c9ccccc9c9ccccc98)cccc67)cc5c4c3)n2)cc1 ; c1ccc(-c2nc(-c3ccccc3)nc(-c3ccc4oc5ccc(-c6cccc7c6oc6cc(-n8c9ccccc9c9ccccc98)ccc67)cc5c4c3)n2)cc1 ; c1ccc(-c2nc(-c3ccccc3)nc(-c3ccc4oc5ccc(-c6cccc7c6oc6ccc(-n8c9ccccc9c9ccccc98)cc67)cc5c4c3)n2)cc1 ; c1ccc(-c2nc(-c3ccccc3)nc(-c3ccc4oc5ccc(-c6cccc7c6oc6cccc(-n8c9ccccc9c9ccccc98)c67)cc5c4c3)n2)cc1]

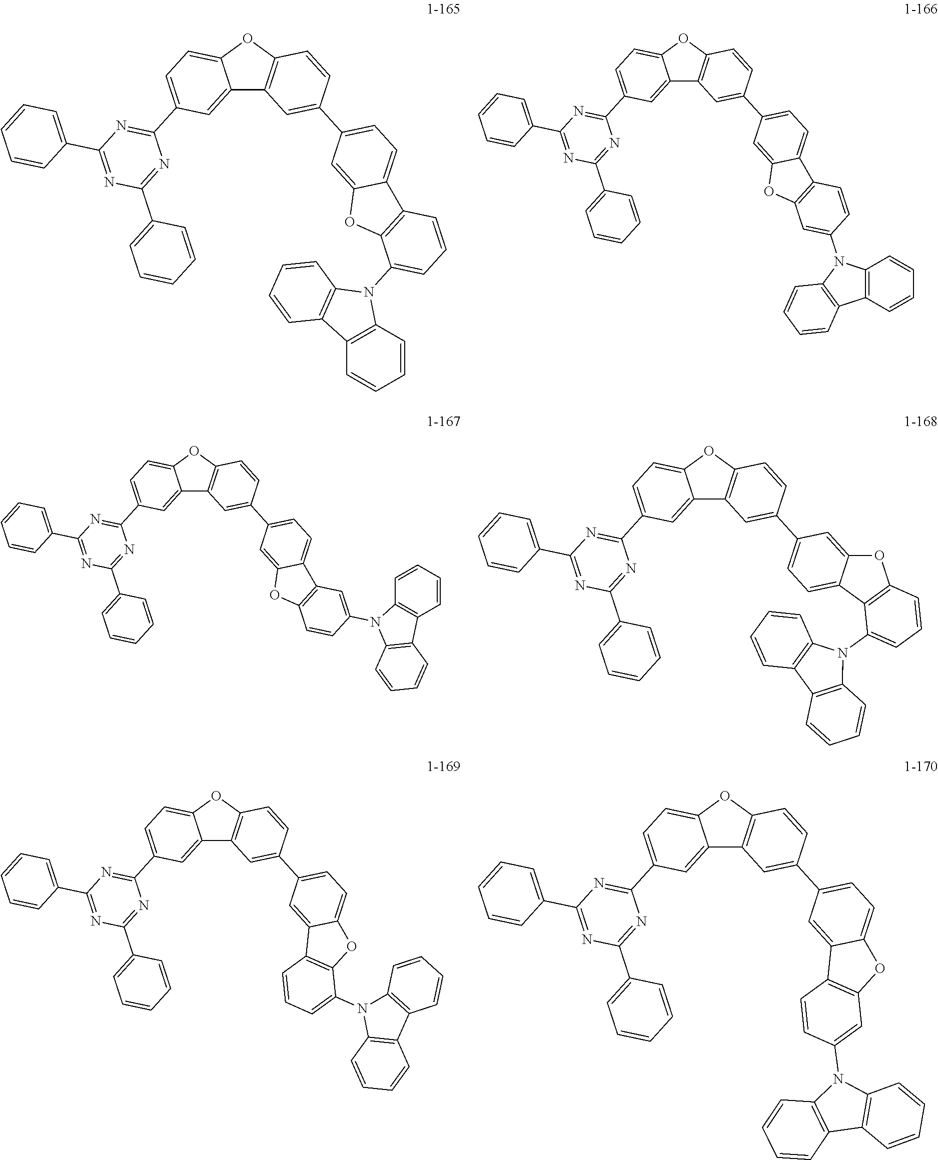

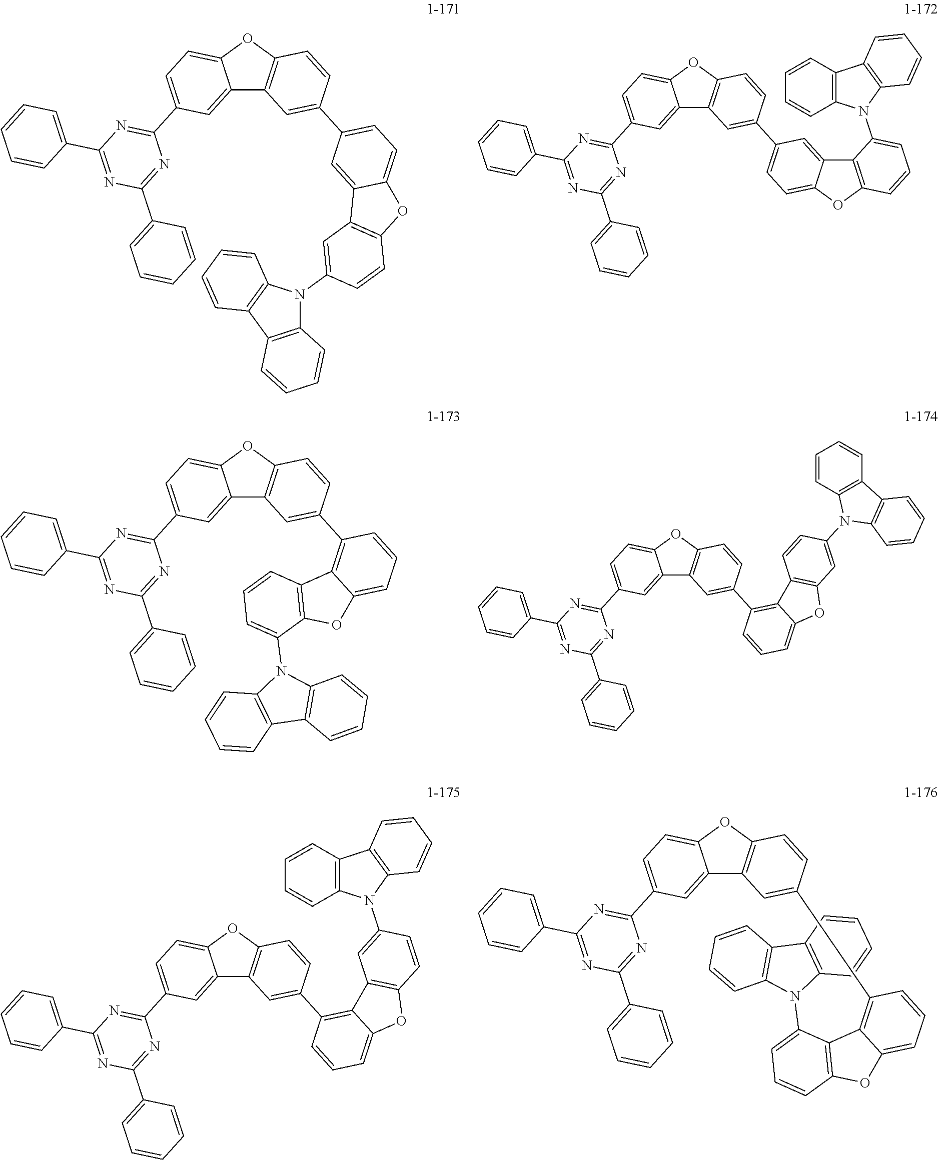

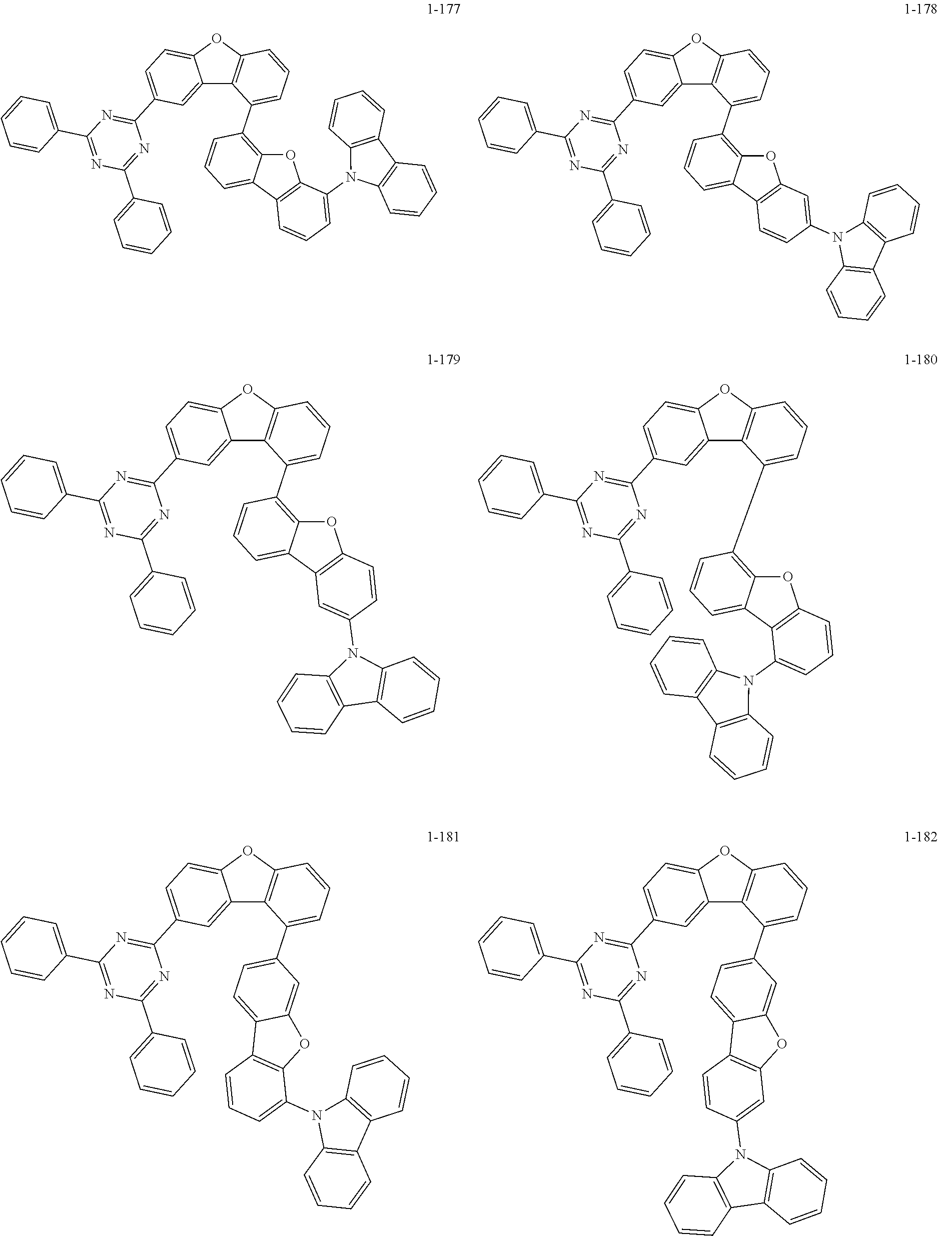

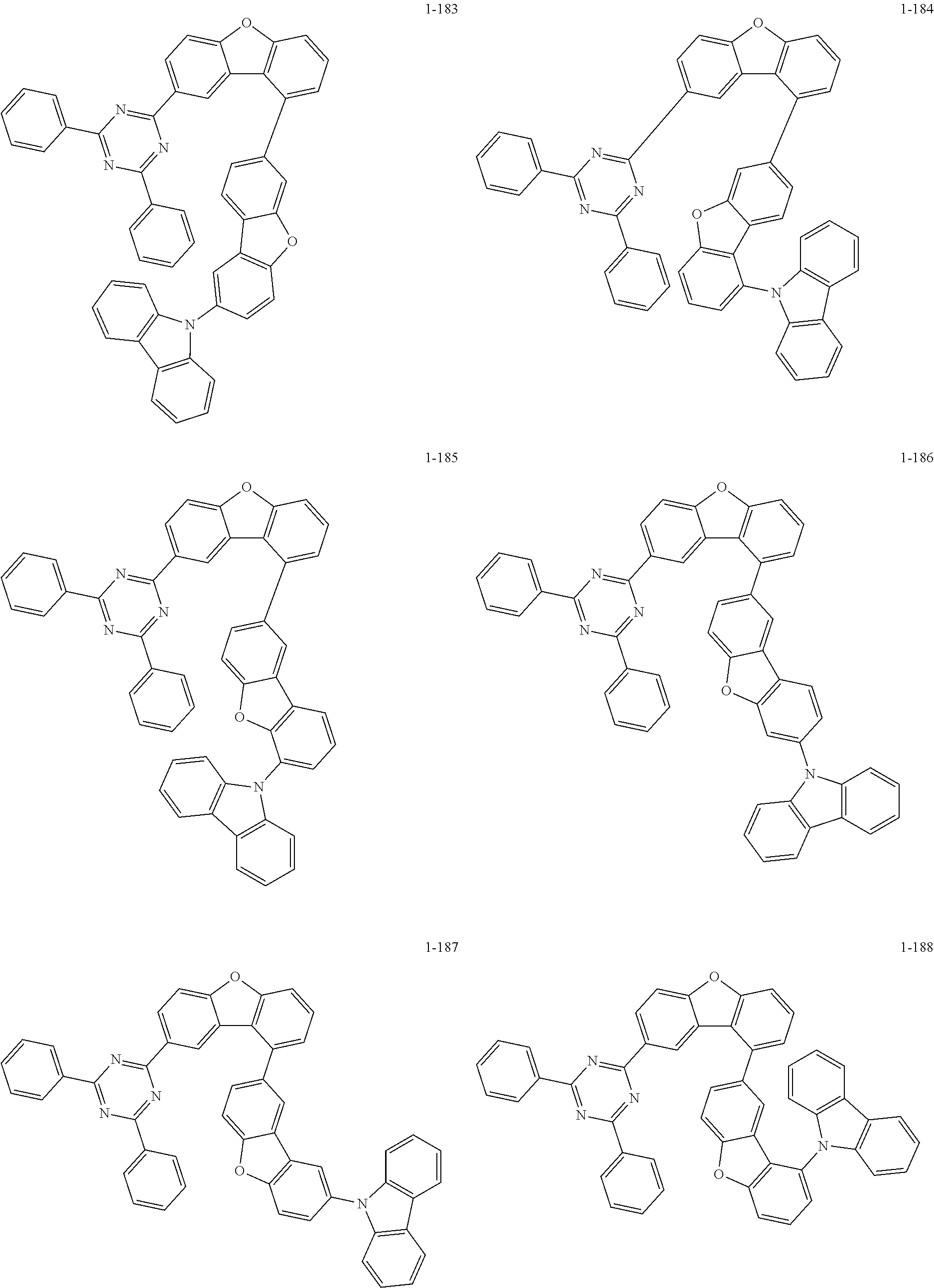

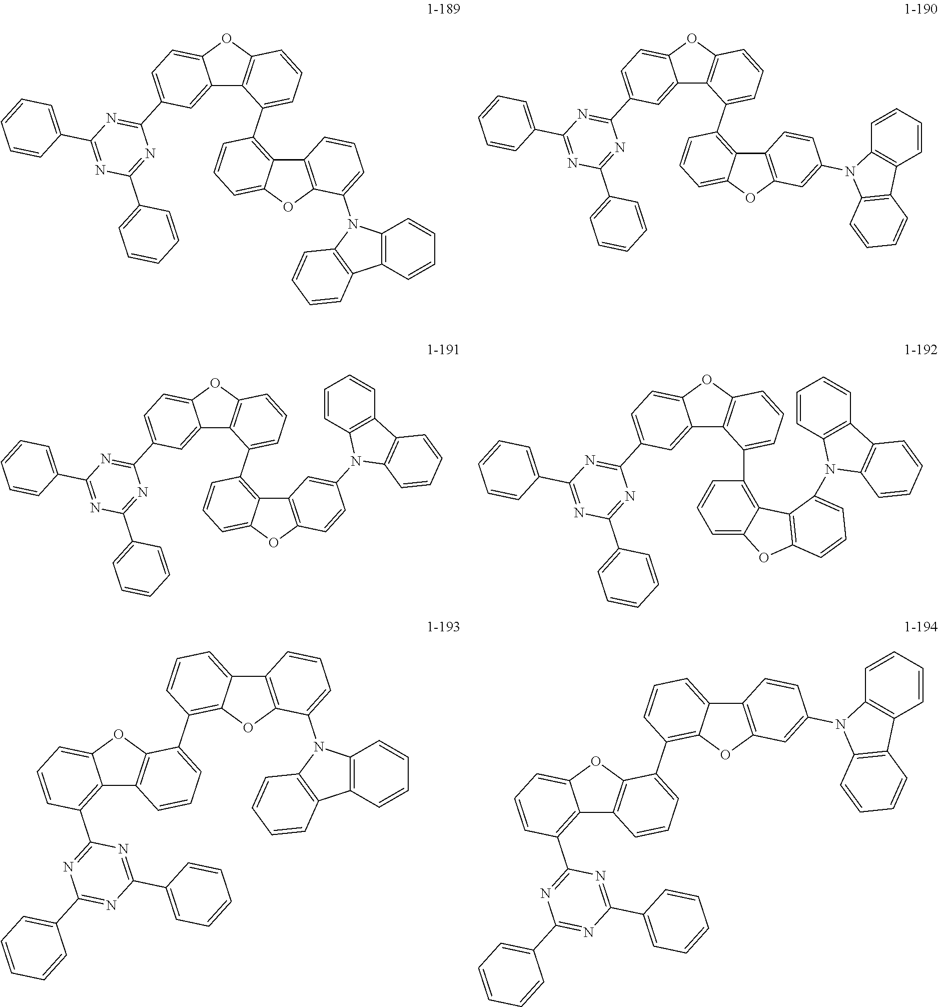

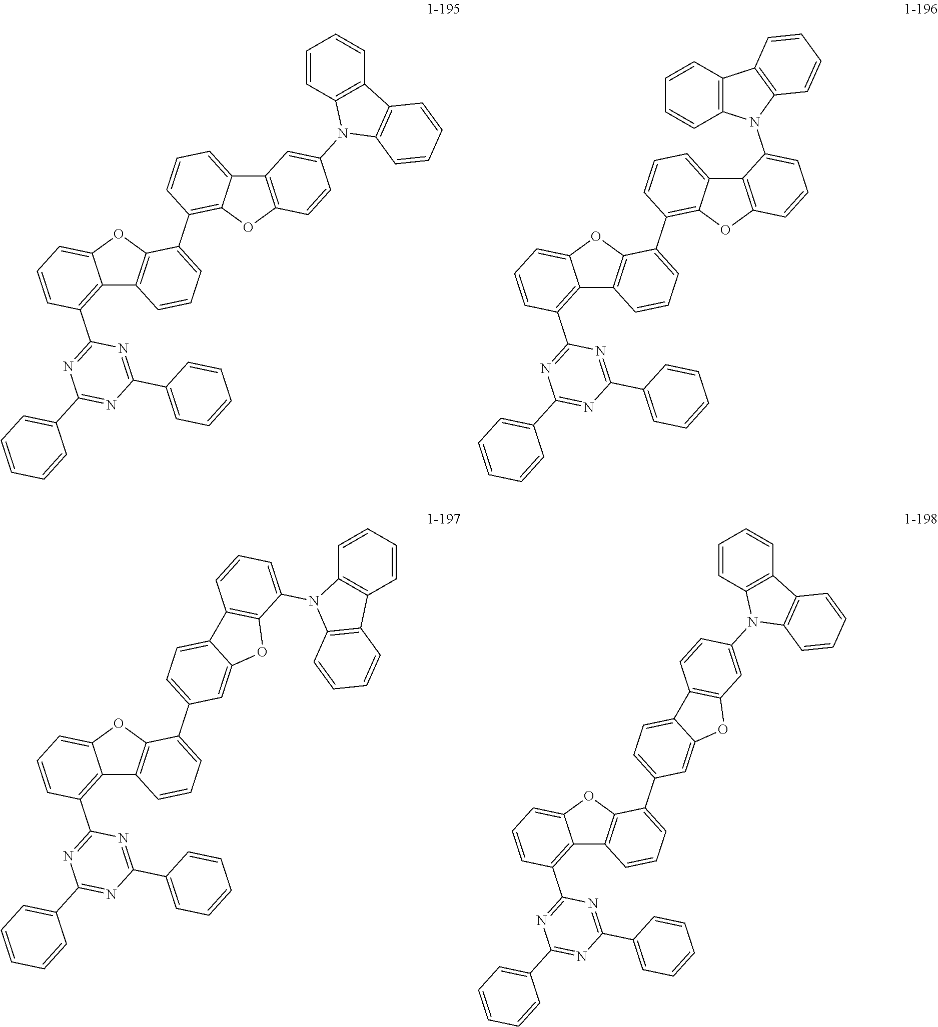 [SMILES: c1ccc(-c2nc(-c3ccccc3)nc(-c3cccc4oc5c(-c6cccc7c6oc6ccc(-n8c9ccccc9c9ccccc98)cc67)cccc5c34)n2)cc1 ; c1ccc(-c2nc(-c3ccccc3)nc(-c3cccc4oc5c(-c6cccc7c6oc6cccc(-n8c9ccccc9c9ccccc98)c67)cccc5c34)n2)cc1 ; c1ccc(-c2nc(-c3ccccc3)nc(-c3cccc4oc5c(-c6ccc7c(c6)oc6c(-n8c9ccccc9c9ccccc98)cccc67)cccc5c34)n2)cc1 ; c1ccc(-c2nc(-c3ccccc3)nc(-c3cccc4oc5c(-c6ccc7c(c6)oc6cc(-n8c9ccccc9c9ccccc98)ccc67)cccc5c34)n2)cc1]

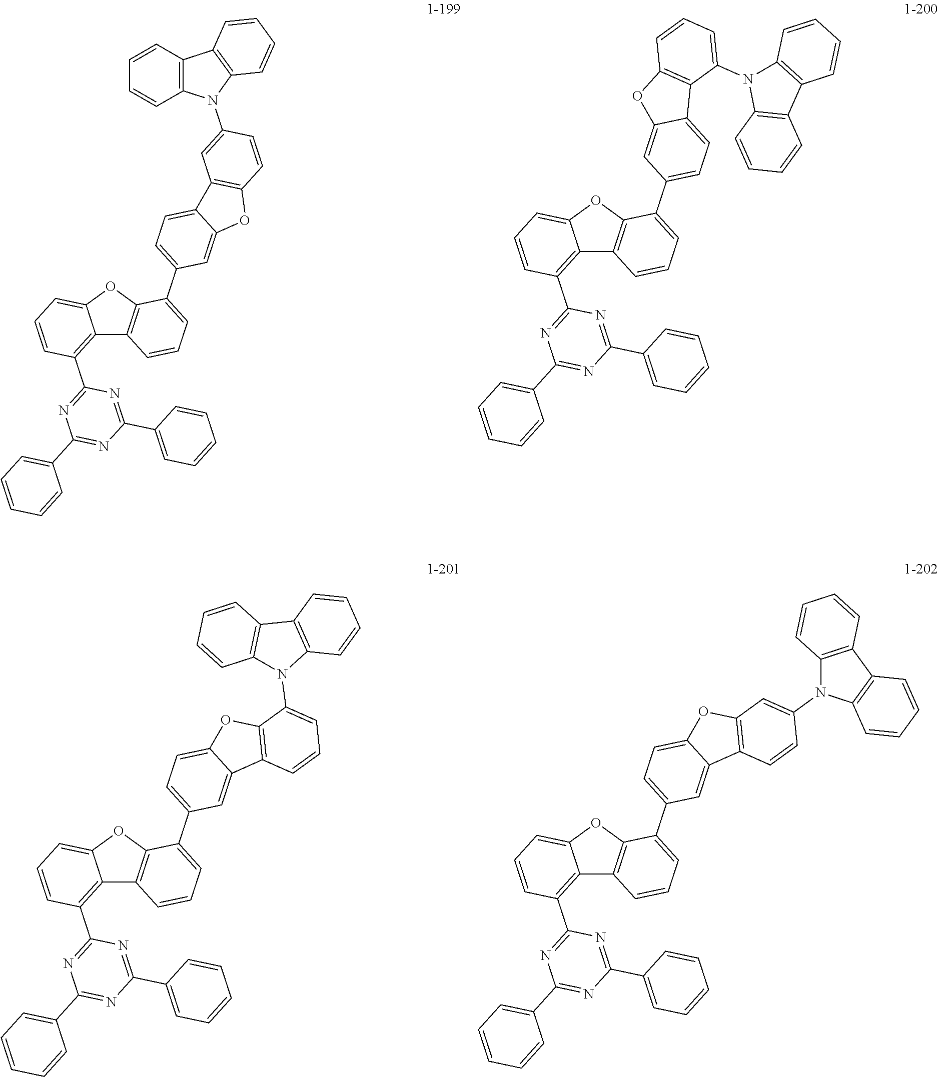 [SMILES: c1ccc(-c2nc(-c3ccccc3)nc(-c3cccc4oc5c(-c6ccc7c(c6)oc6ccc(-n8c9ccccc9c9ccccc98)cc67)cccc5c34)n2)cc1 ; c1ccc(-c2nc(-c3ccccc3)nc(-c3cccc4oc5c(-c6ccc7c(c6)oc6cccc(-n8c9ccccc9c9ccccc98)c67)cccc5c34)n2)cc1 ; c1ccc(-c2nc(-c3ccccc3)nc(-c3cccc4oc5c(-c6ccc7oc8c(-n9c%10ccccc%10c%10ccccc%109)cccc8c7c6)cccc5c34)n2)cc1 ; c1ccc(-c2nc(-c3ccccc3)nc(-c3cccc4oc5c(-c6ccc7oc8cc(-n9c%10ccccc%10c%10ccccc%109)ccc8c7c6)cccc5c34)n2)cc1]

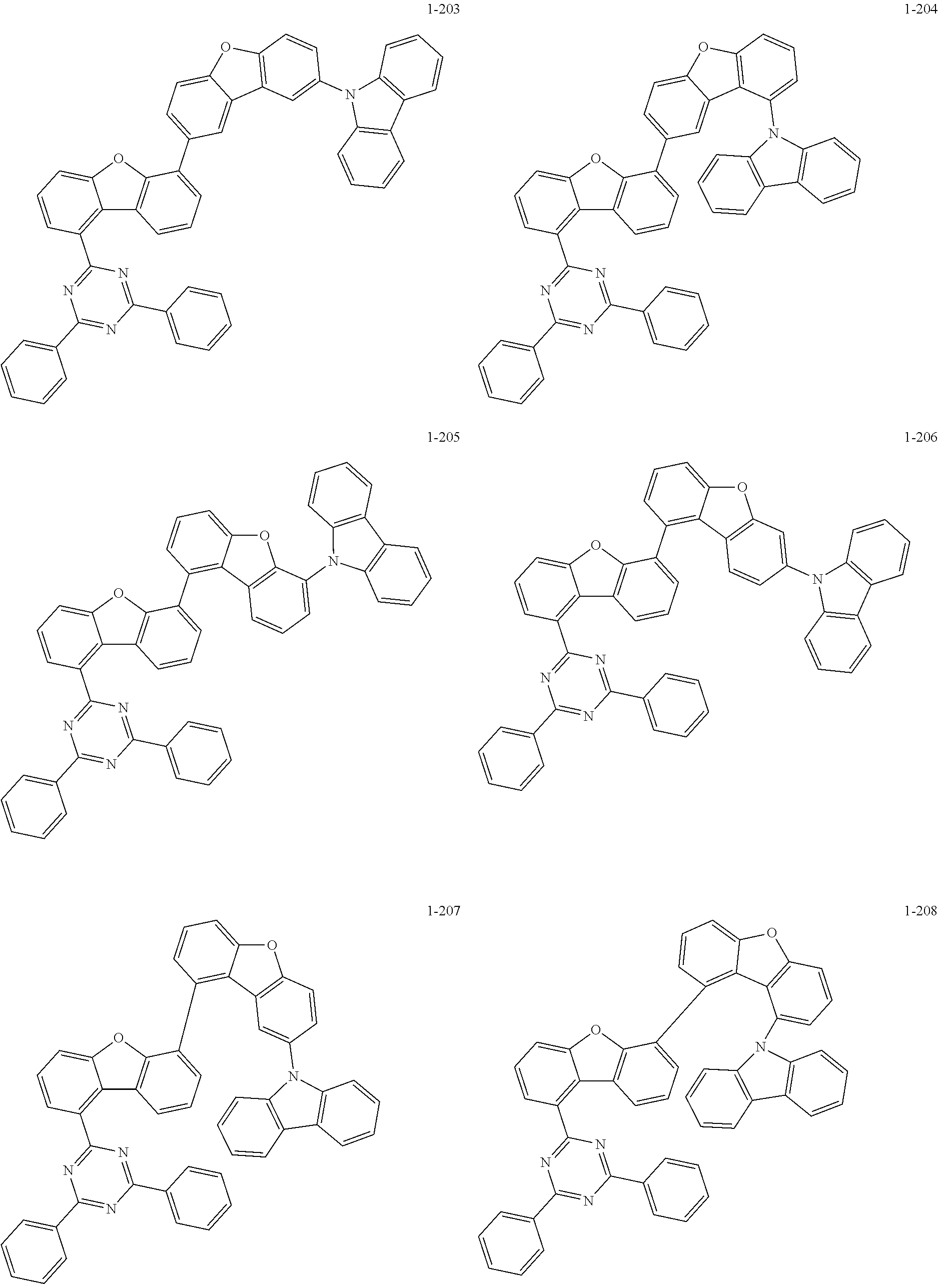

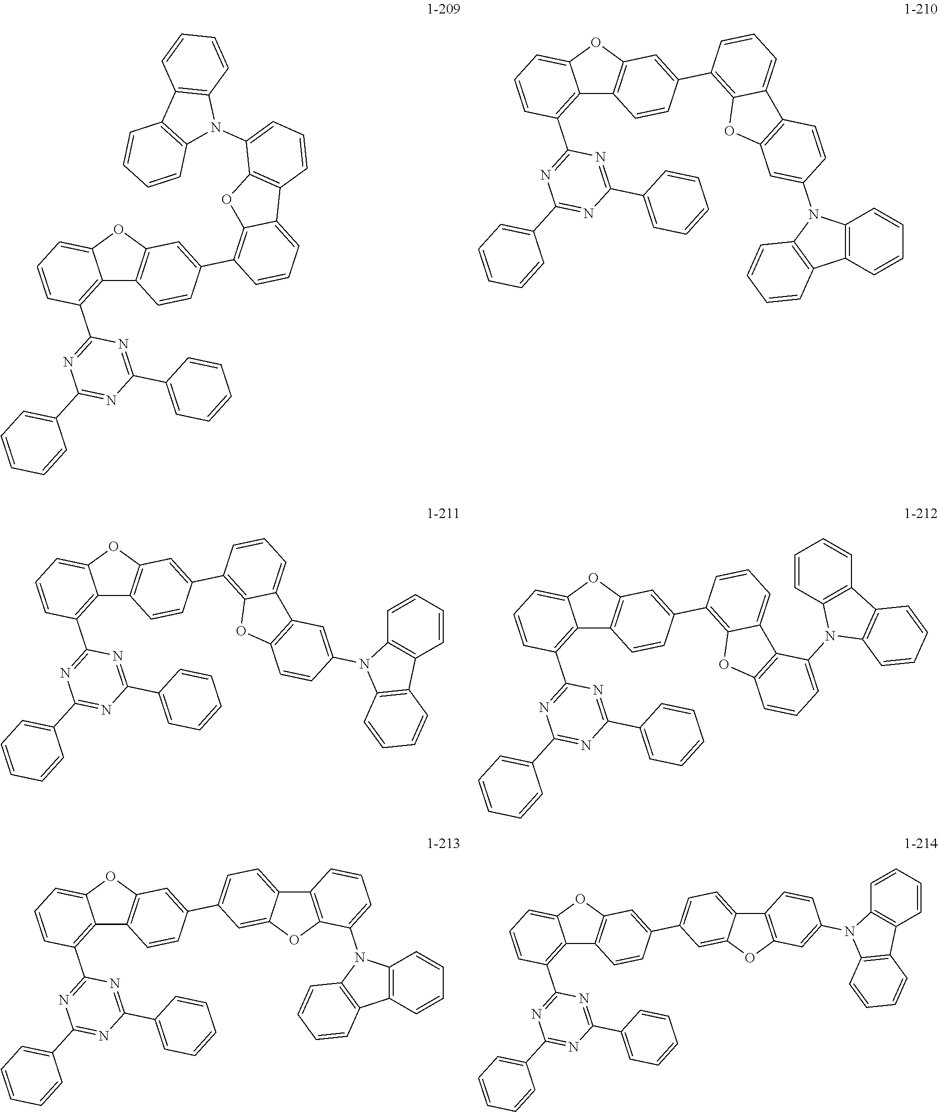

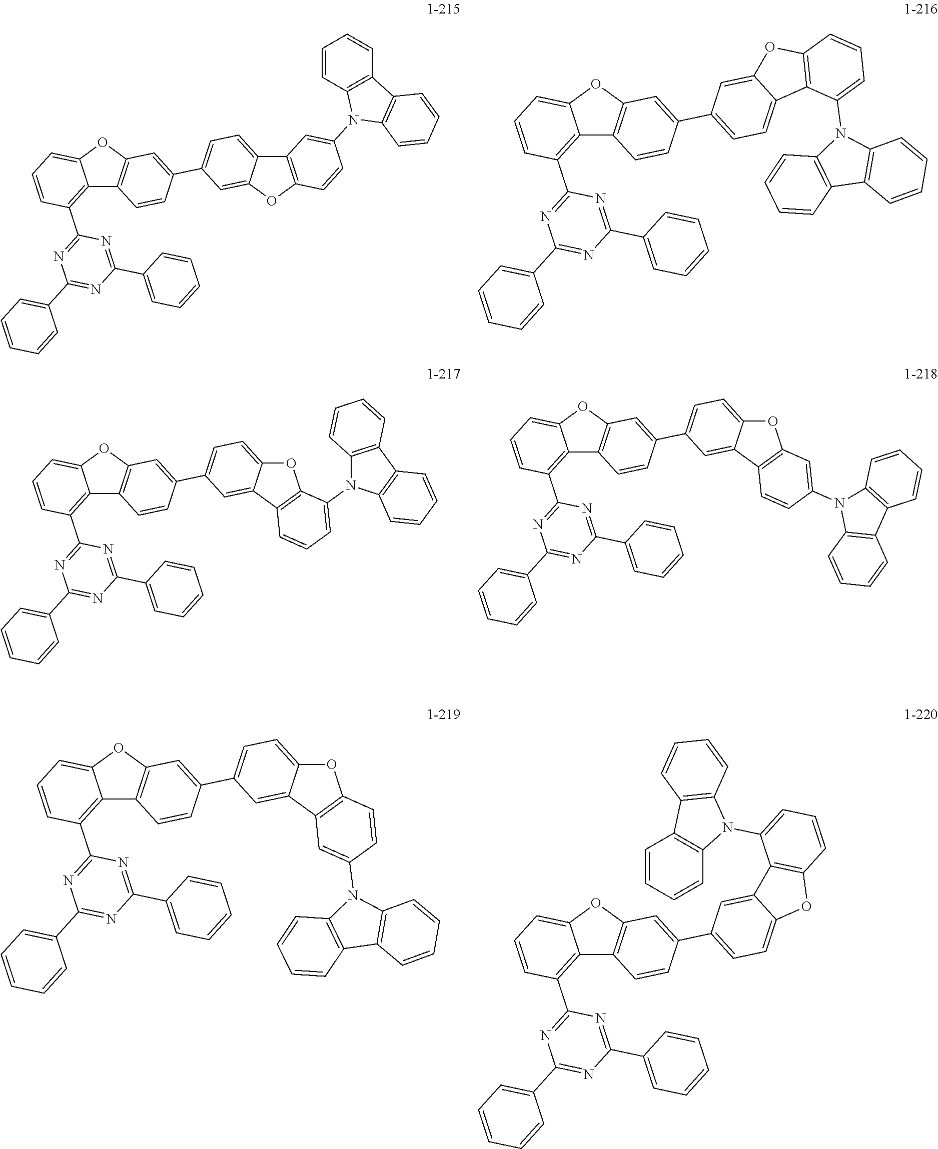

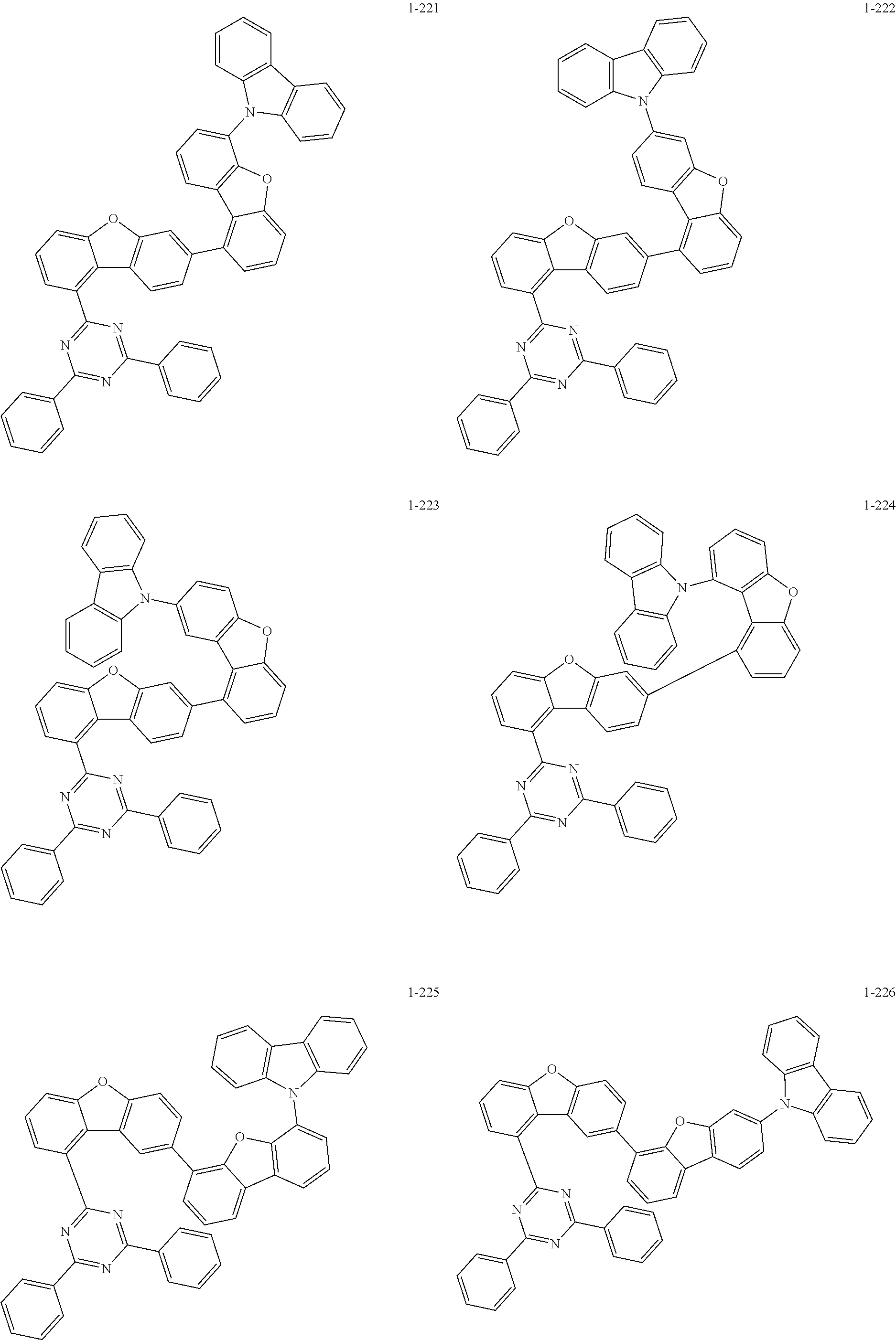

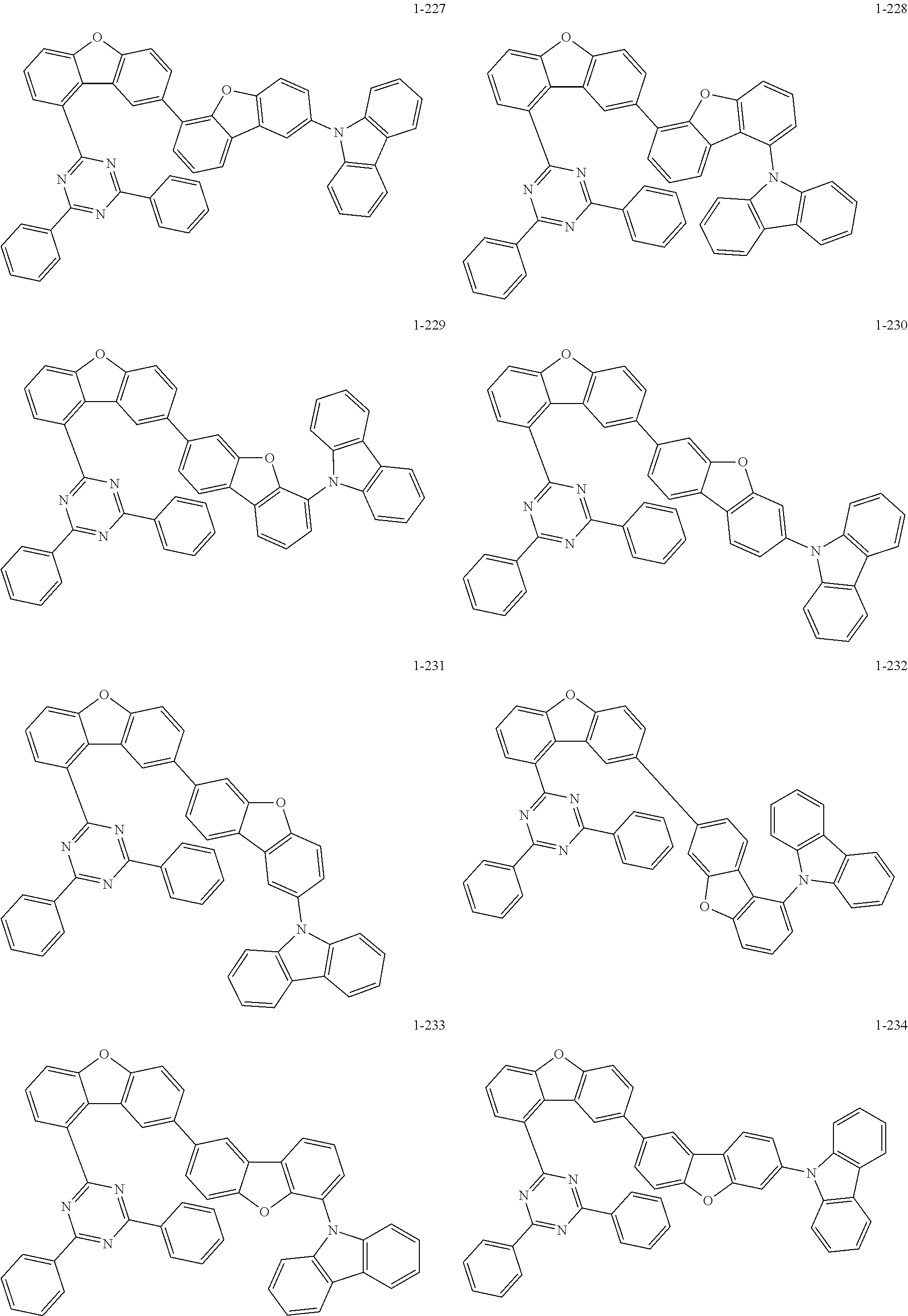

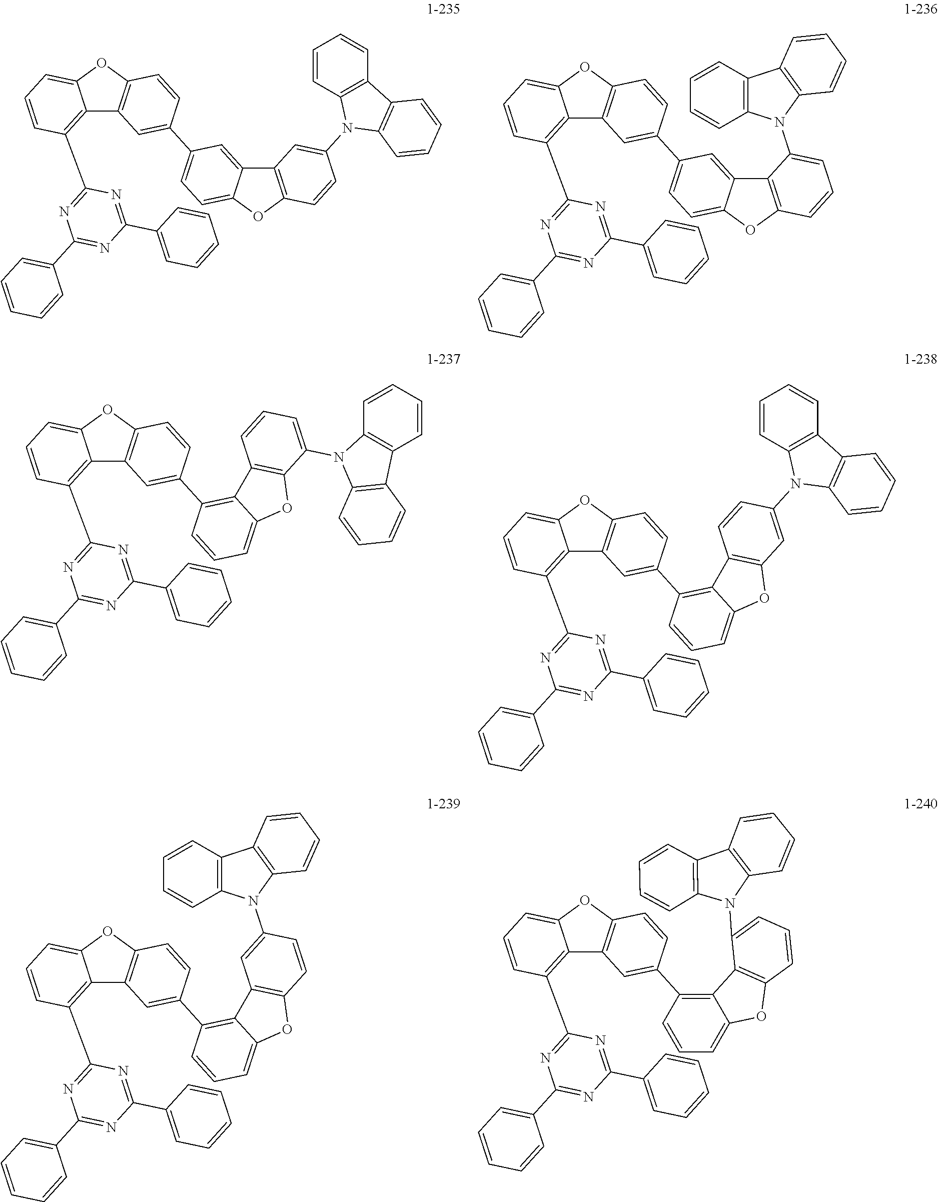

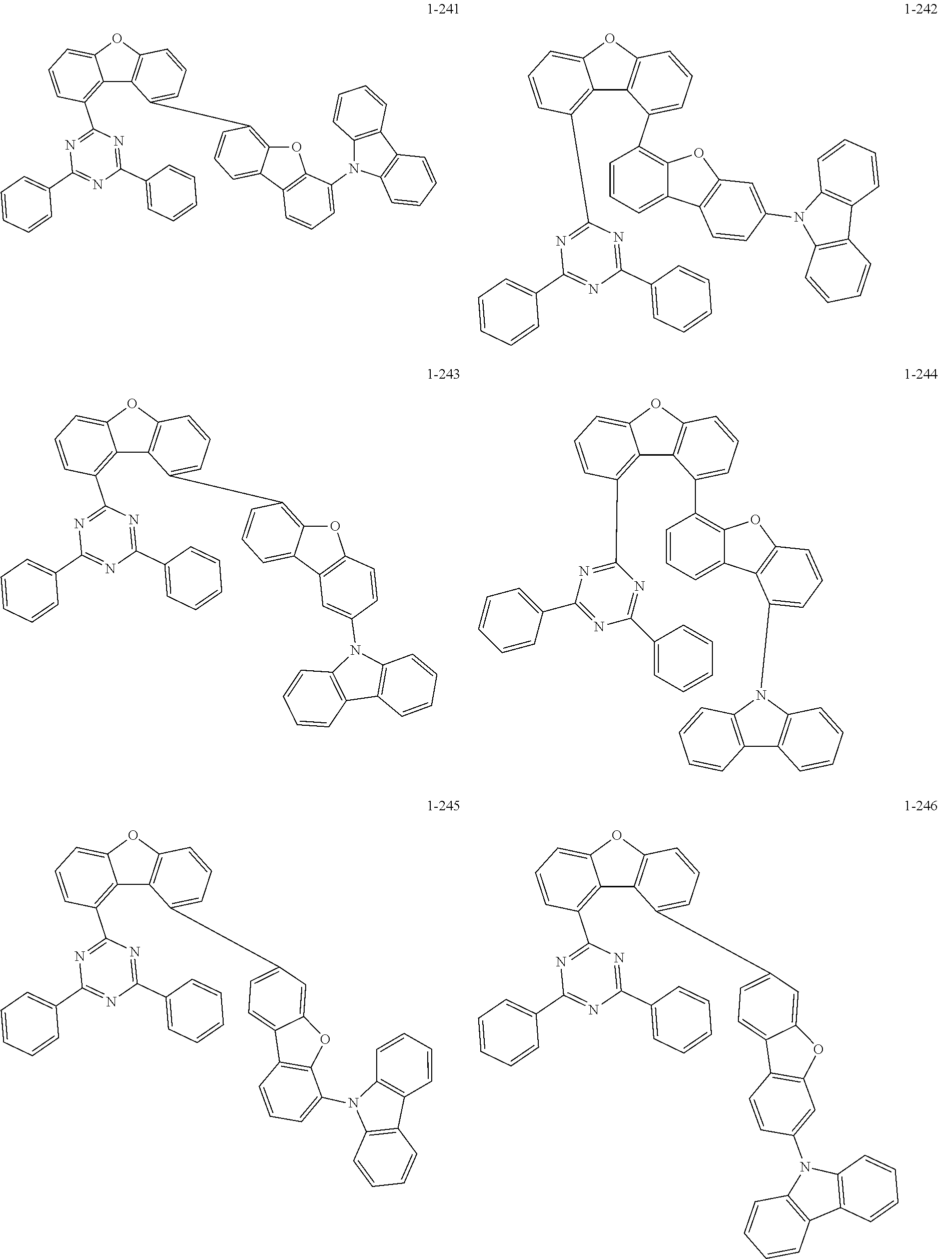

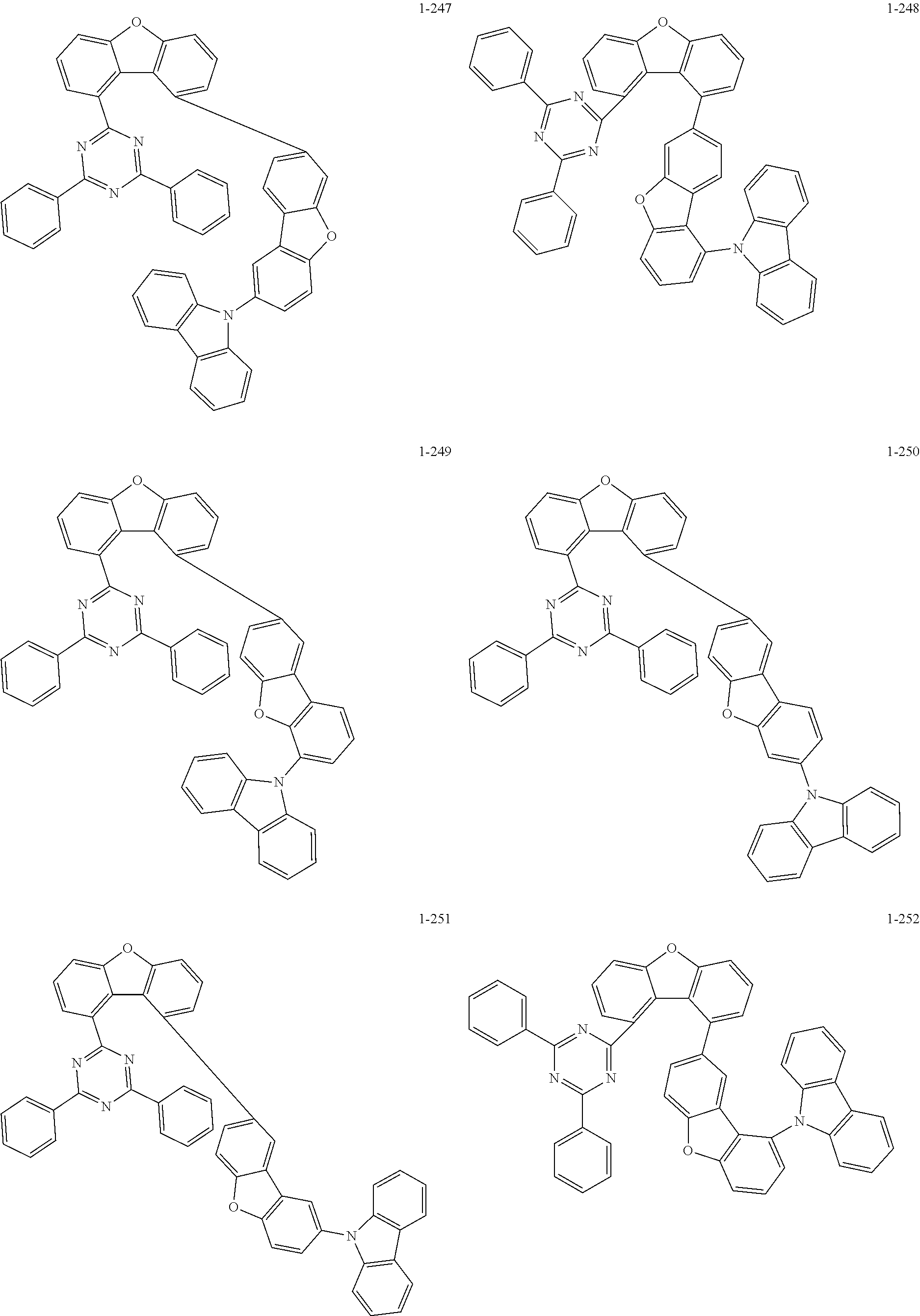

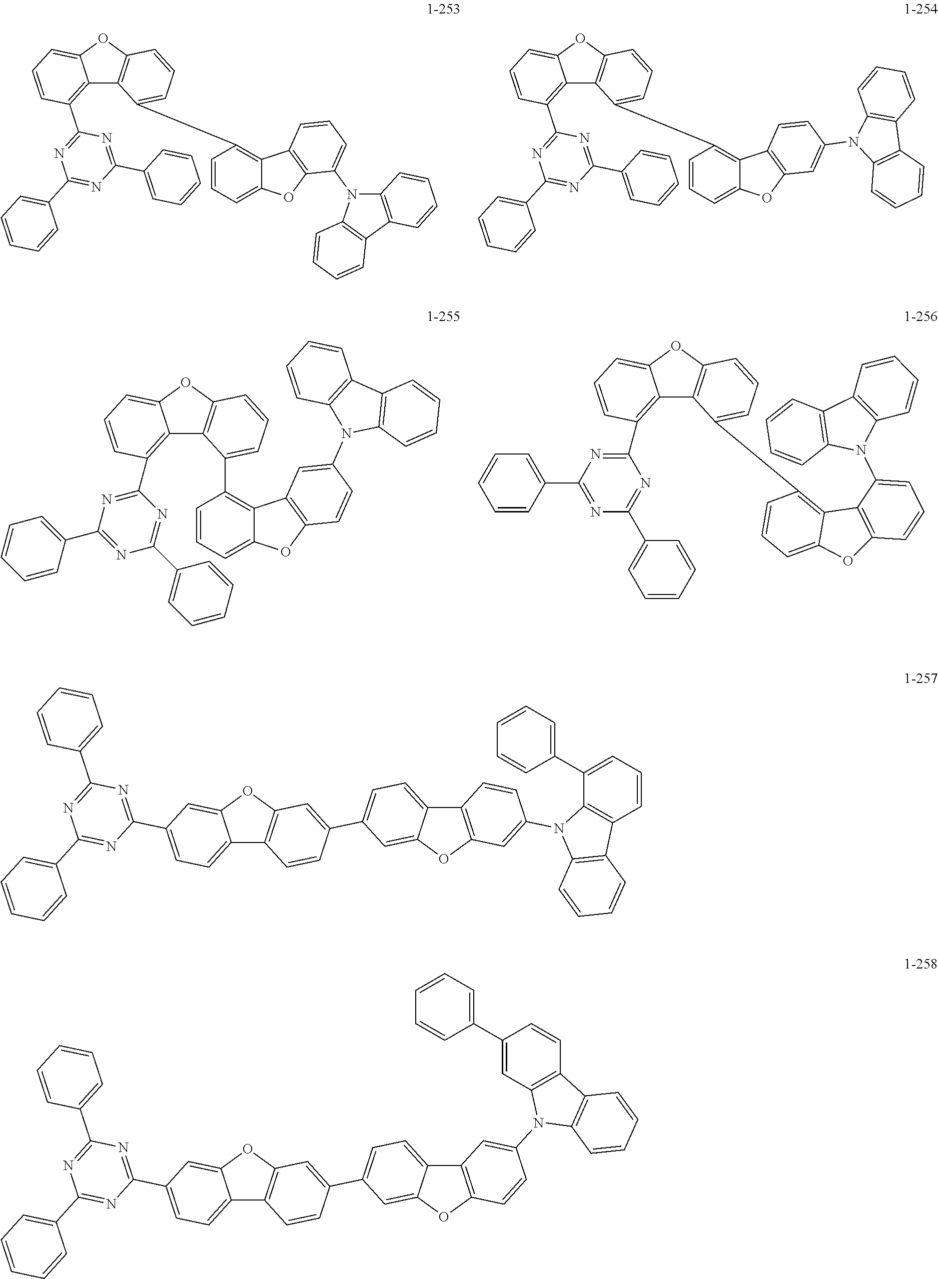

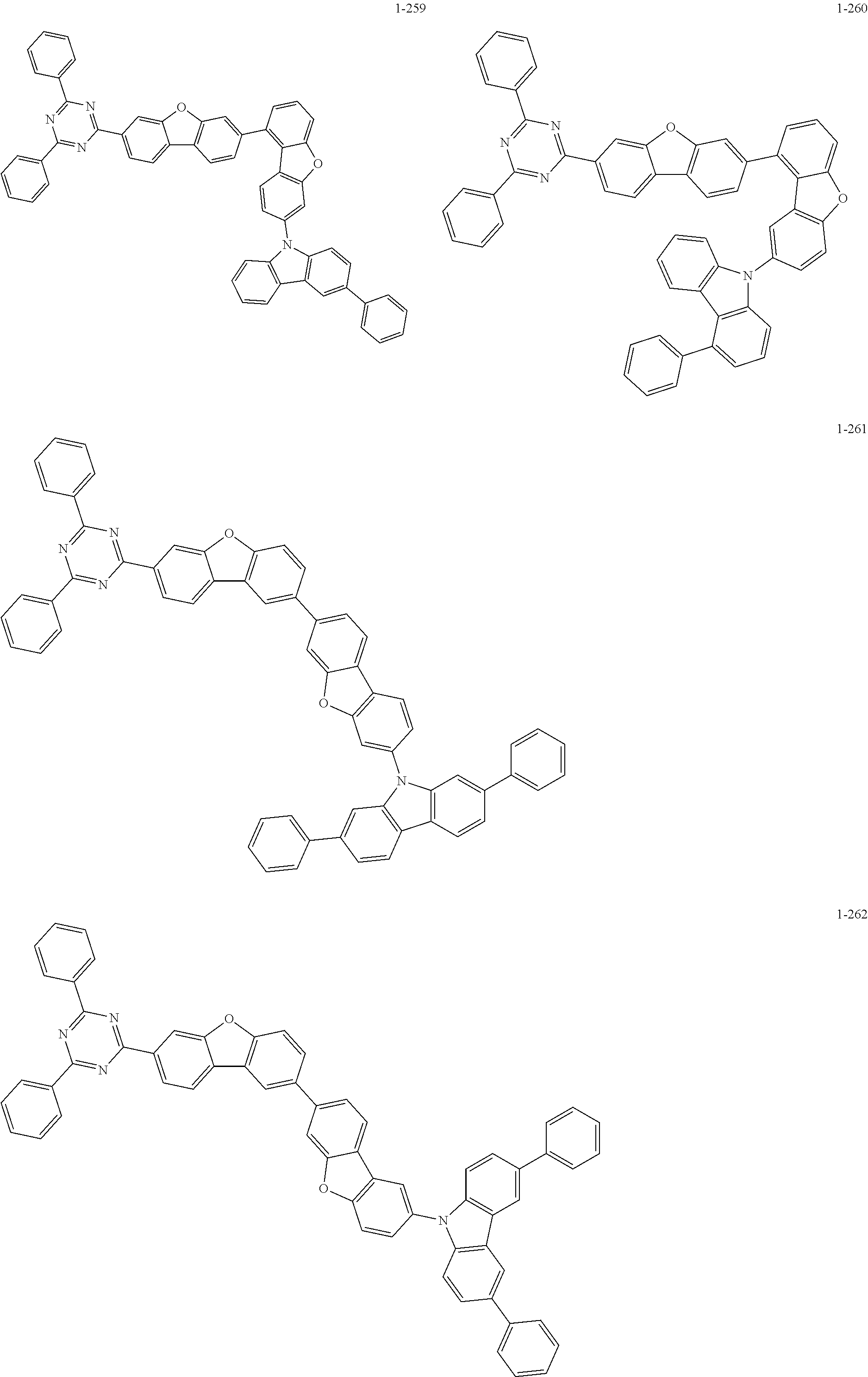 [SMILES: c1ccc(-c2ccc3c(c2)c2ccccc2n3-c2ccc3c(c2)oc2cccc(-c4ccc5c(c4)oc4cc(-c6nc(-c7ccccc7)nc(-c7ccccc7)n6)ccc45)c23)cc1 ; c1ccc(-c2nc(-c3ccccc3)nc(-c3ccc4c(c3)oc3cc(-c5cccc6oc7ccc(-n8c9ccccc9c9c(-c%10ccccc%10)cccc98)cc7c56)ccc34)n2)cc1 ; c1ccc(-c2ccc3c4ccc(-c5ccccc5)cc4n(-c4ccc5c(c4)oc4cc(-c6ccc7oc8cc(-c9nc(-c%10ccccc%10)nc(-c%10ccccc%10)n9)ccc8c7c6)ccc45)c3c2)cc1 ; c1ccc(-c2ccc3c(c2)c2cc(-c4ccccc4)ccc2n3-c2ccc3oc4cc(-c5ccc6oc7cc(-c8nc(-c9ccccc9)nc(-c9ccccc9)n8)ccc7c6c5)ccc4c3c2)cc1]

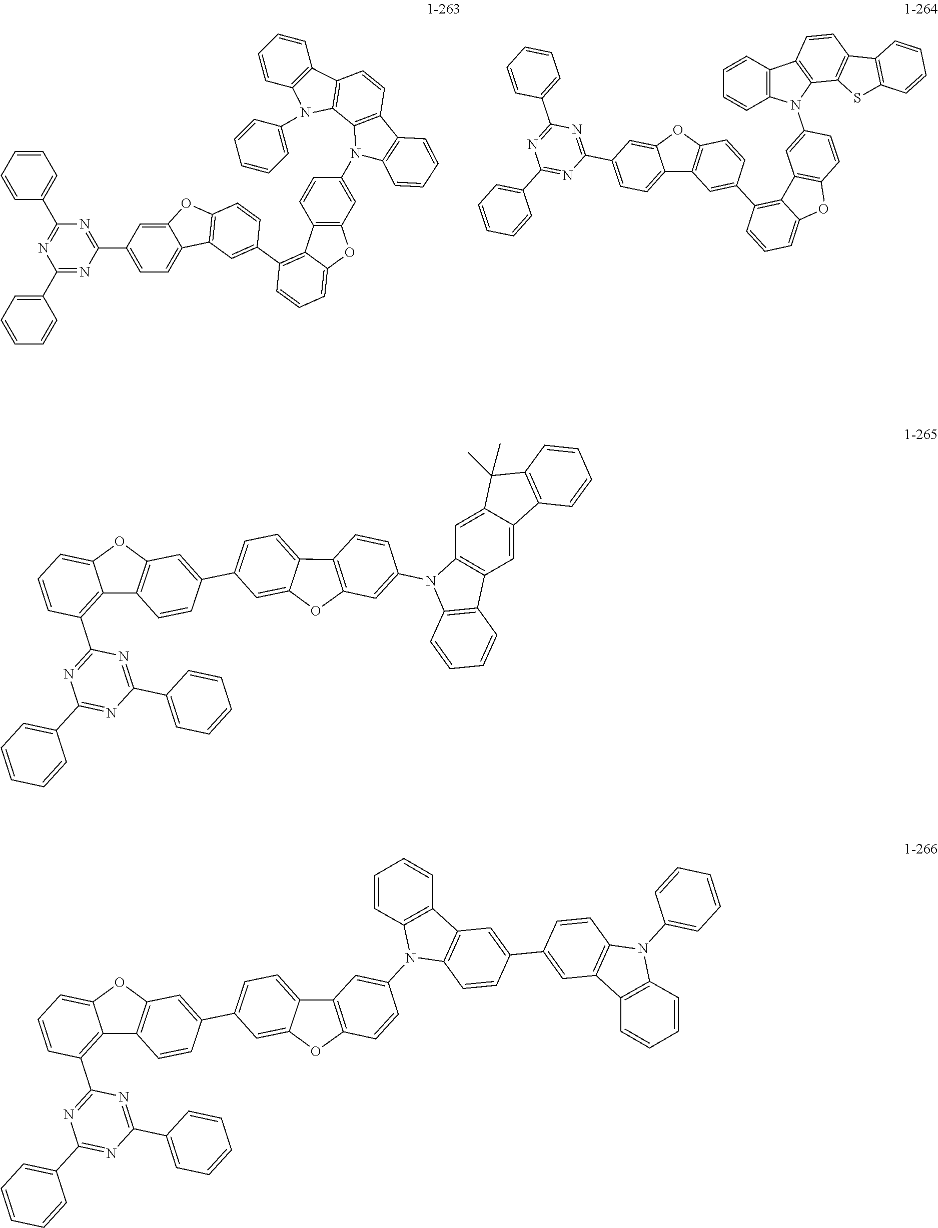

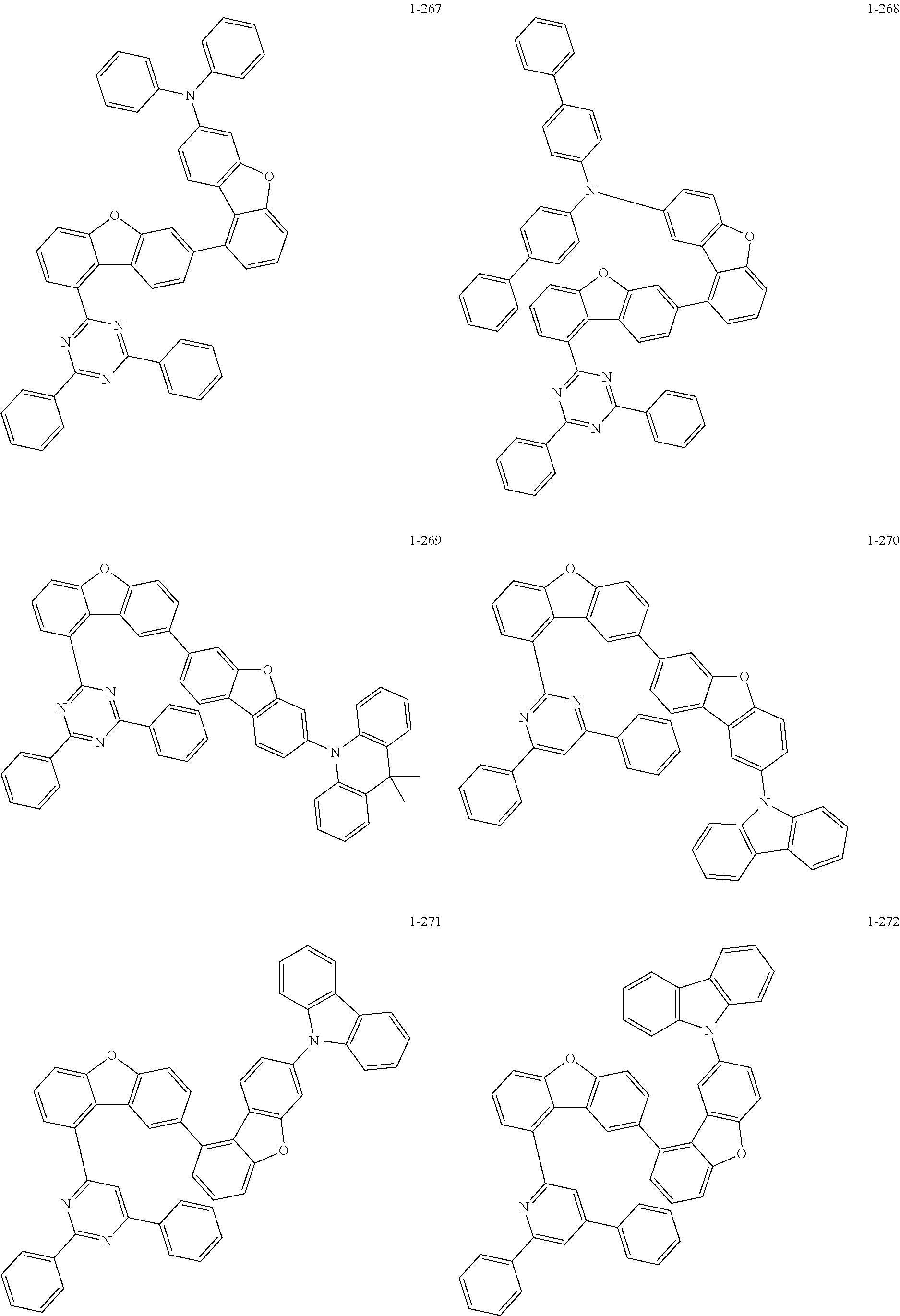

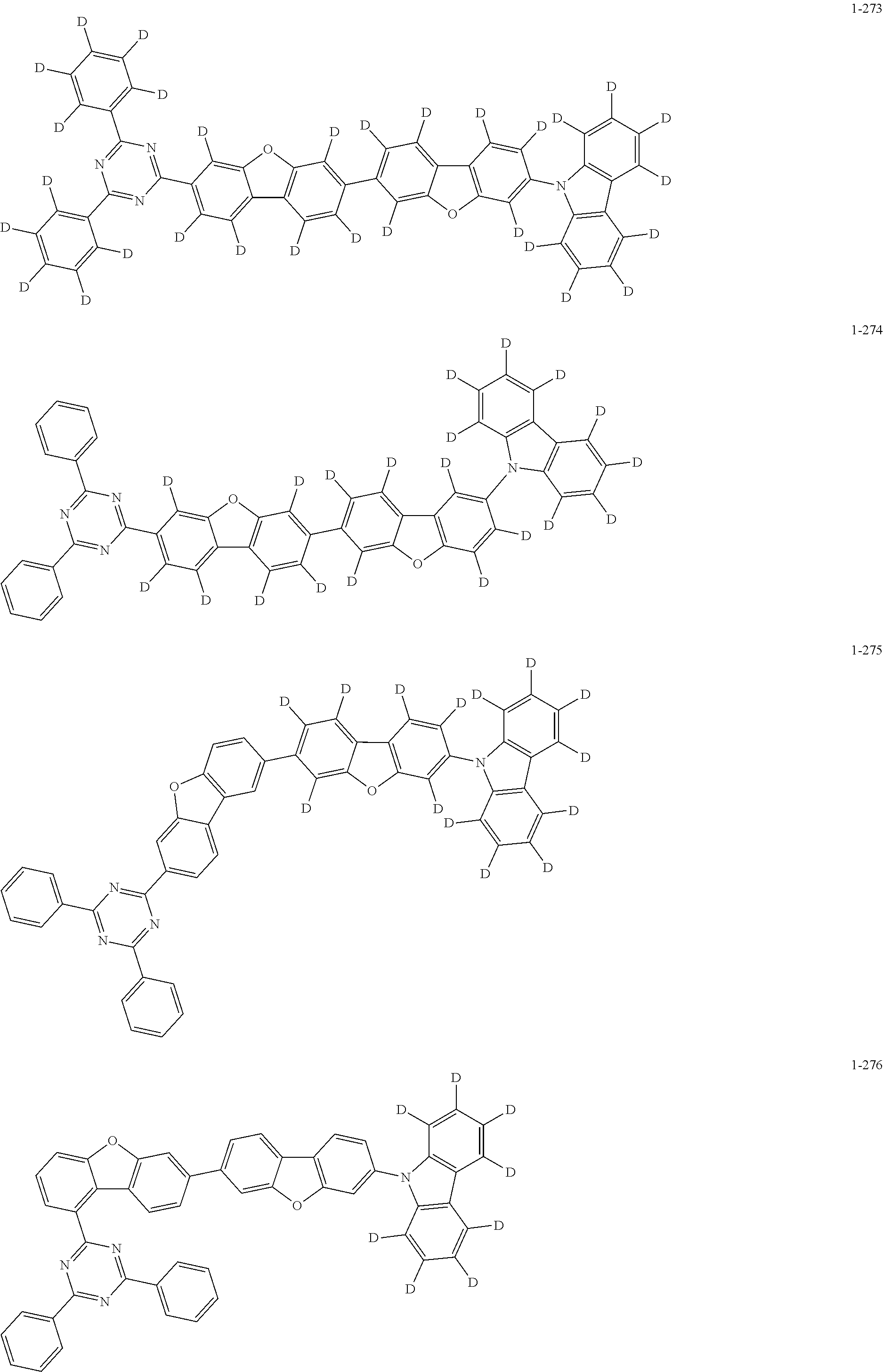

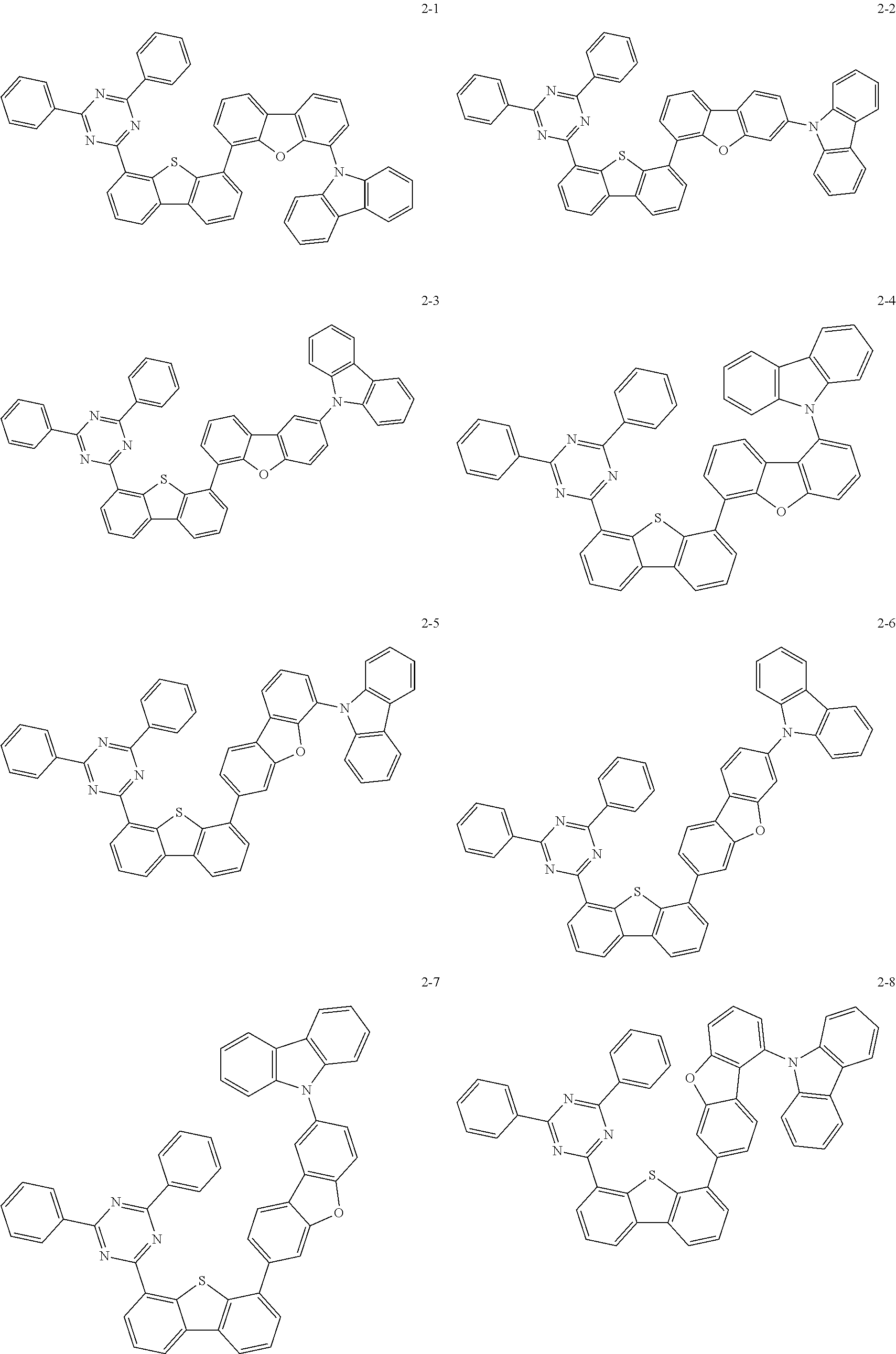

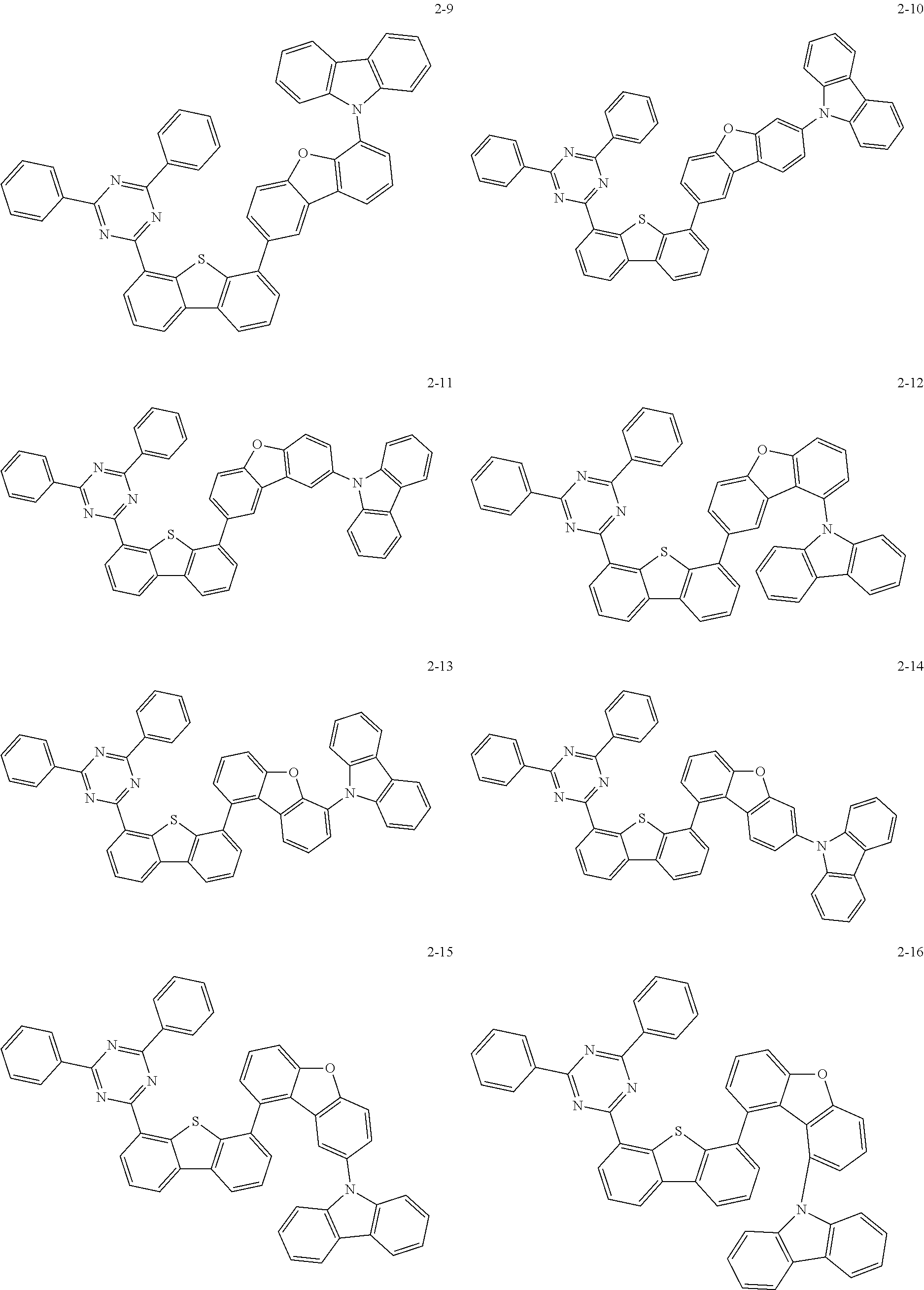

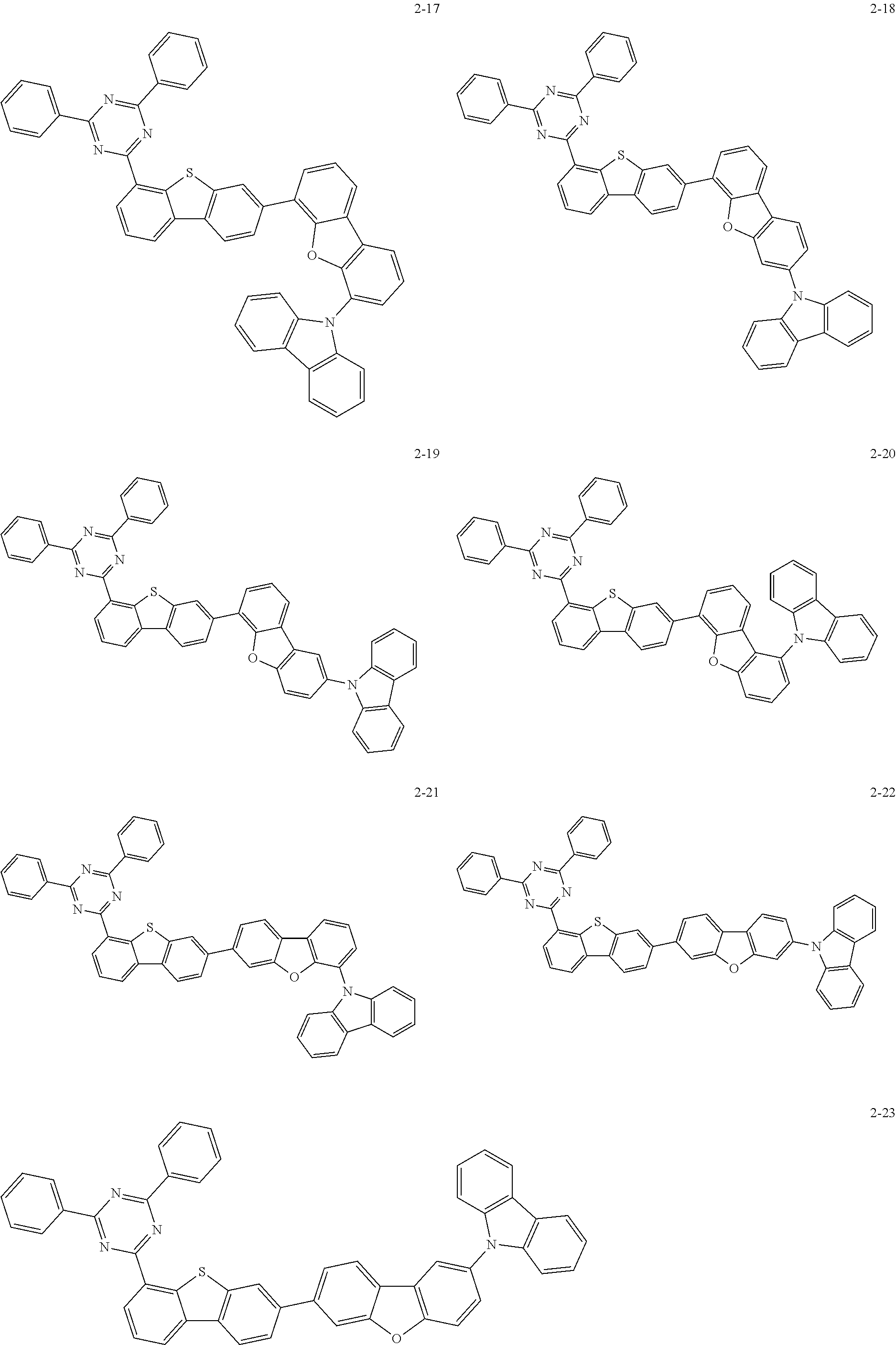

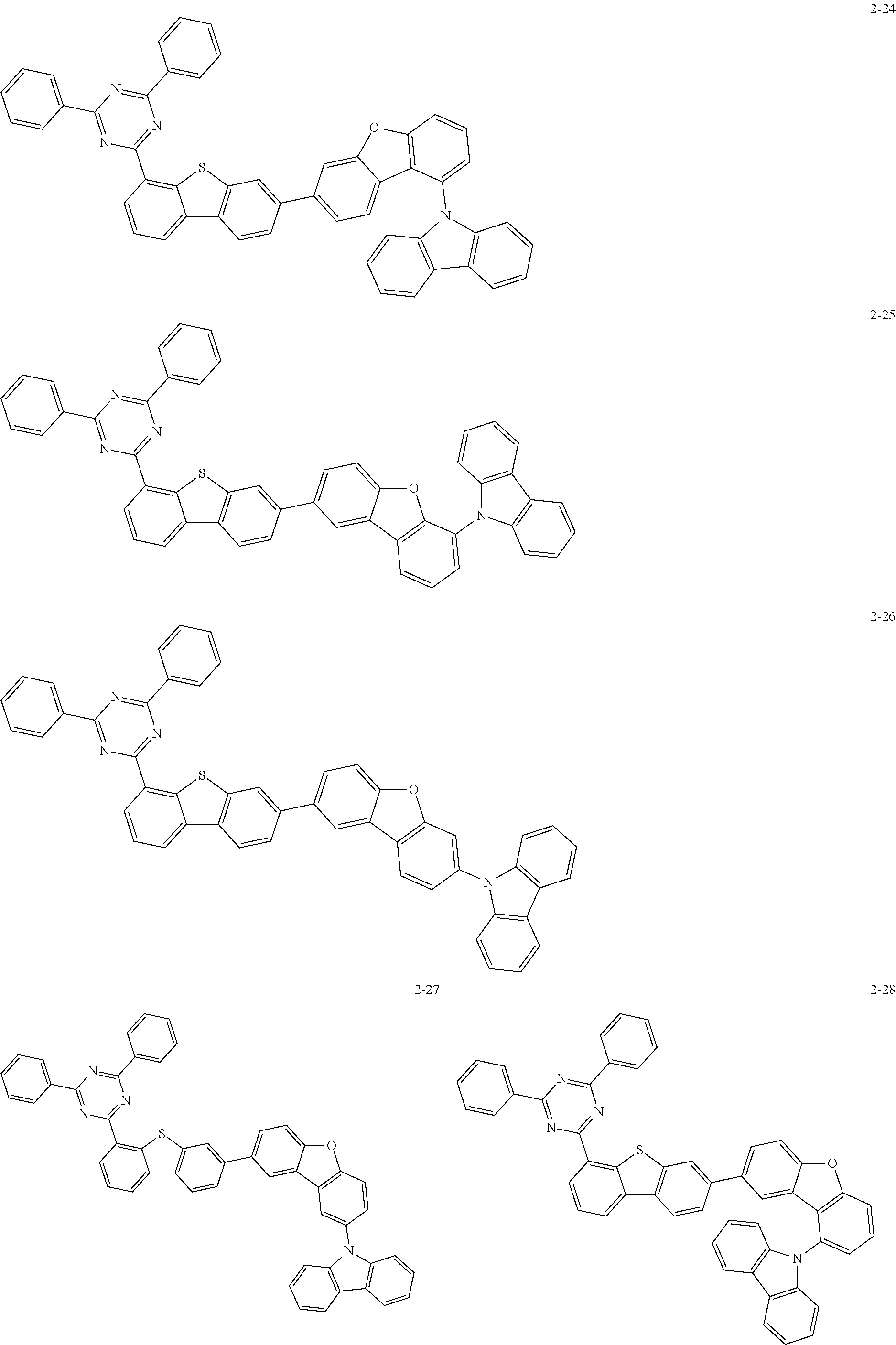 [SMILES: c1ccc(-c2nc(-c3ccccc3)nc(-c3cccc4c3sc3cc(-c5ccc6c(c5)oc5cccc(-n7c8ccccc8c8ccccc87)c56)ccc34)n2)cc1 ; c1ccc(-c2nc(-c3ccccc3)nc(-c3cccc4c3sc3cc(-c5ccc6oc7c(-n8c9ccccc9c9ccccc98)cccc7c6c5)ccc34)n2)cc1 ; c1ccc(-c2nc(-c3ccccc3)nc(-c3cccc4c3sc3cc(-c5ccc6oc7cc(-n8c9ccccc9c9ccccc98)ccc7c6c5)ccc34)n2)cc1 ; c1ccc(-c2nc(-c3ccccc3)nc(-c3cccc4c3sc3cc(-c5ccc6oc7ccc(-n8c9ccccc9c9ccccc98)cc7c6c5)ccc34)n2)cc1 ; c1ccc(-c2nc(-c3ccccc3)nc(-c3cccc4c3sc3cc(-c5ccc6oc7cccc(-n8c9ccccc9c9ccccc98)c7c6c5)ccc34)n2)cc1]

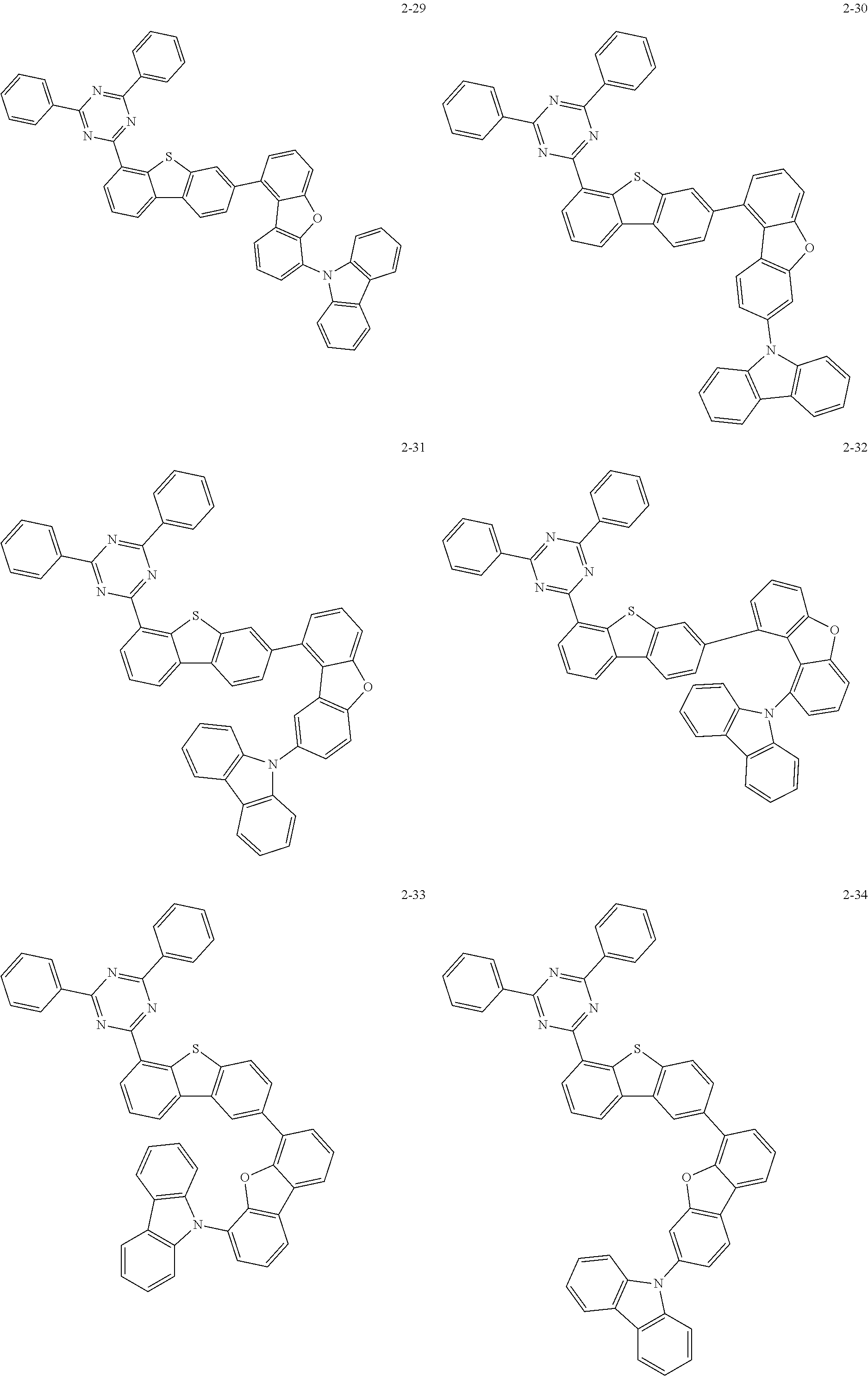

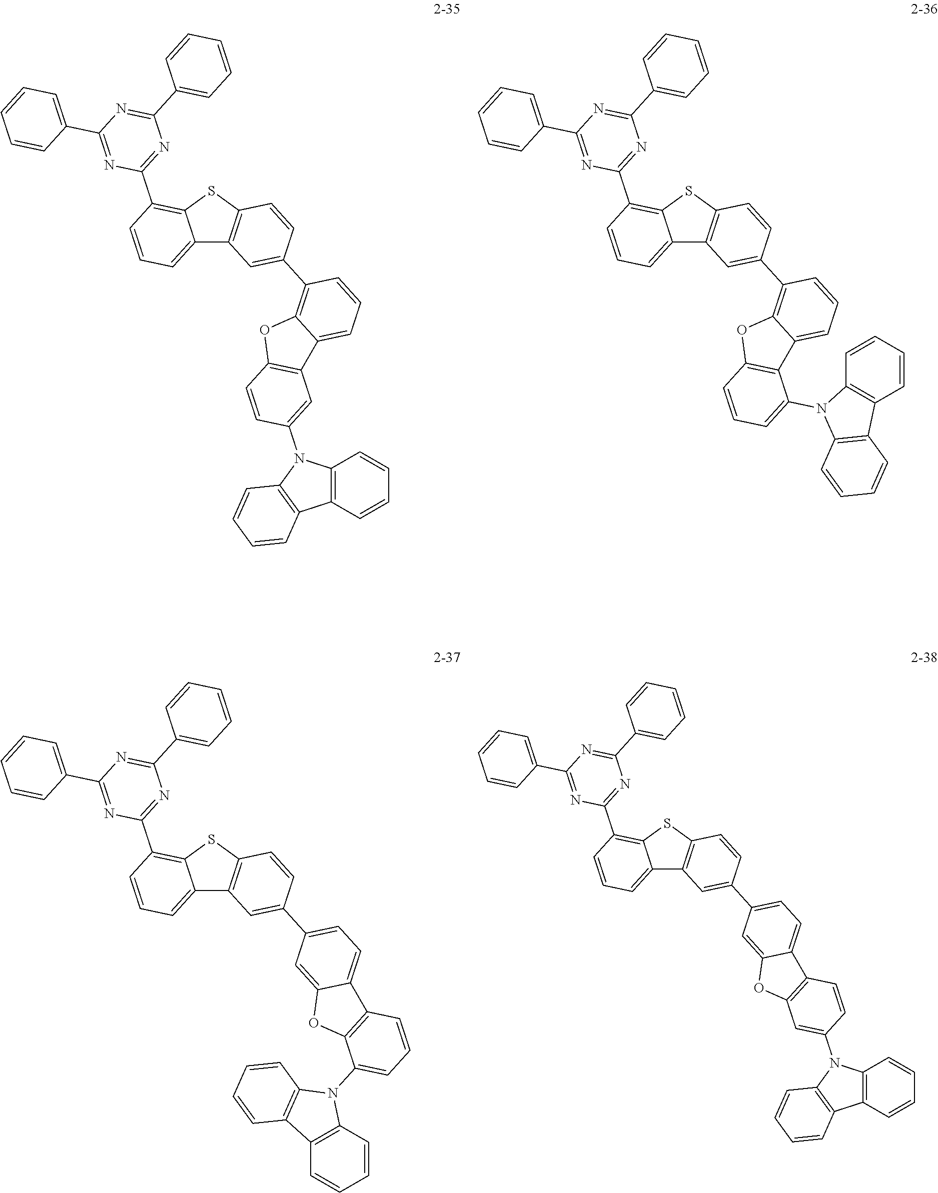 [SMILES: c1ccc(-c2nc(-c3ccccc3)nc(-c3cccc4c3sc3ccc(-c5cccc6c5oc5ccc(-n7c8ccccc8c8ccccc87)cc56)cc34)n2)cc1 ; c1ccc(-c2nc(-c3ccccc3)nc(-c3cccc4c3sc3ccc(-c5cccc6c5oc5cccc(-n7c8ccccc8c8ccccc87)c56)cc34)n2)cc1 ; c1ccc(-c2nc(-c3ccccc3)nc(-c3cccc4c3sc3ccc(-c5ccc6c(c5)oc5c(-n7c8ccccc8c8ccccc87)cccc56)cc34)n2)cc1 ; c1ccc(-c2nc(-c3ccccc3)nc(-c3cccc4c3sc3ccc(-c5ccc6c(c5)oc5cc(-n7c8ccccc8c8ccccc87)ccc56)cc34)n2)cc1]

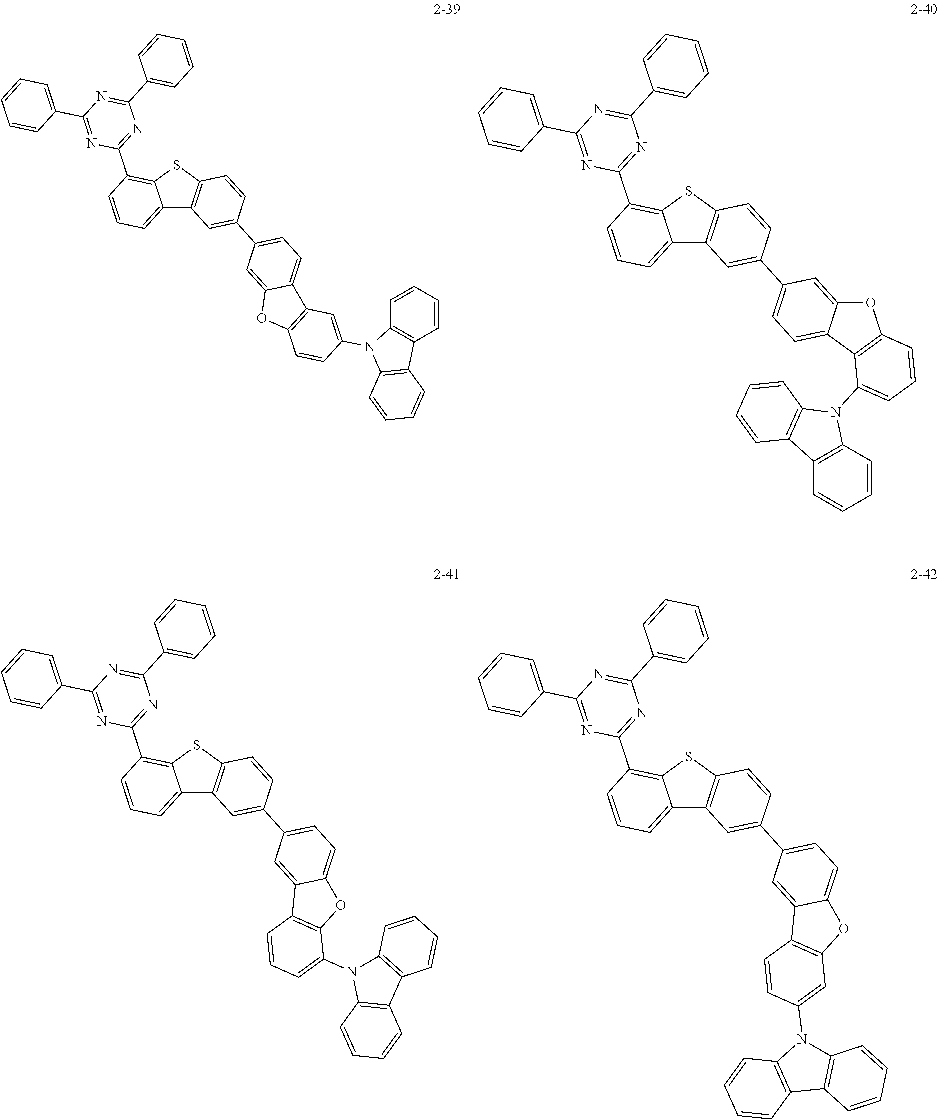 [SMILES: c1ccc(-c2nc(-c3ccccc3)nc(-c3cccc4c3sc3ccc(-c5ccc6c(c5)oc5ccc(-n7c8ccccc8c8ccccc87)cc56)cc34)n2)cc1 ; c1ccc(-c2nc(-c3ccccc3)nc(-c3cccc4c3sc3ccc(-c5ccc6c(c5)oc5cccc(-n7c8ccccc8c8ccccc87)c56)cc34)n2)cc1 ; c1ccc(-c2nc(-c3ccccc3)nc(-c3cccc4c3sc3ccc(-c5ccc6oc7c(-n8c9ccccc9c9ccccc98)cccc7c6c5)cc34)n2)cc1 ; c1ccc(-c2nc(-c3ccccc3)nc(-c3cccc4c3sc3ccc(-c5ccc6oc7cc(-n8c9ccccc9c9ccccc98)ccc7c6c5)cc34)n2)cc1]

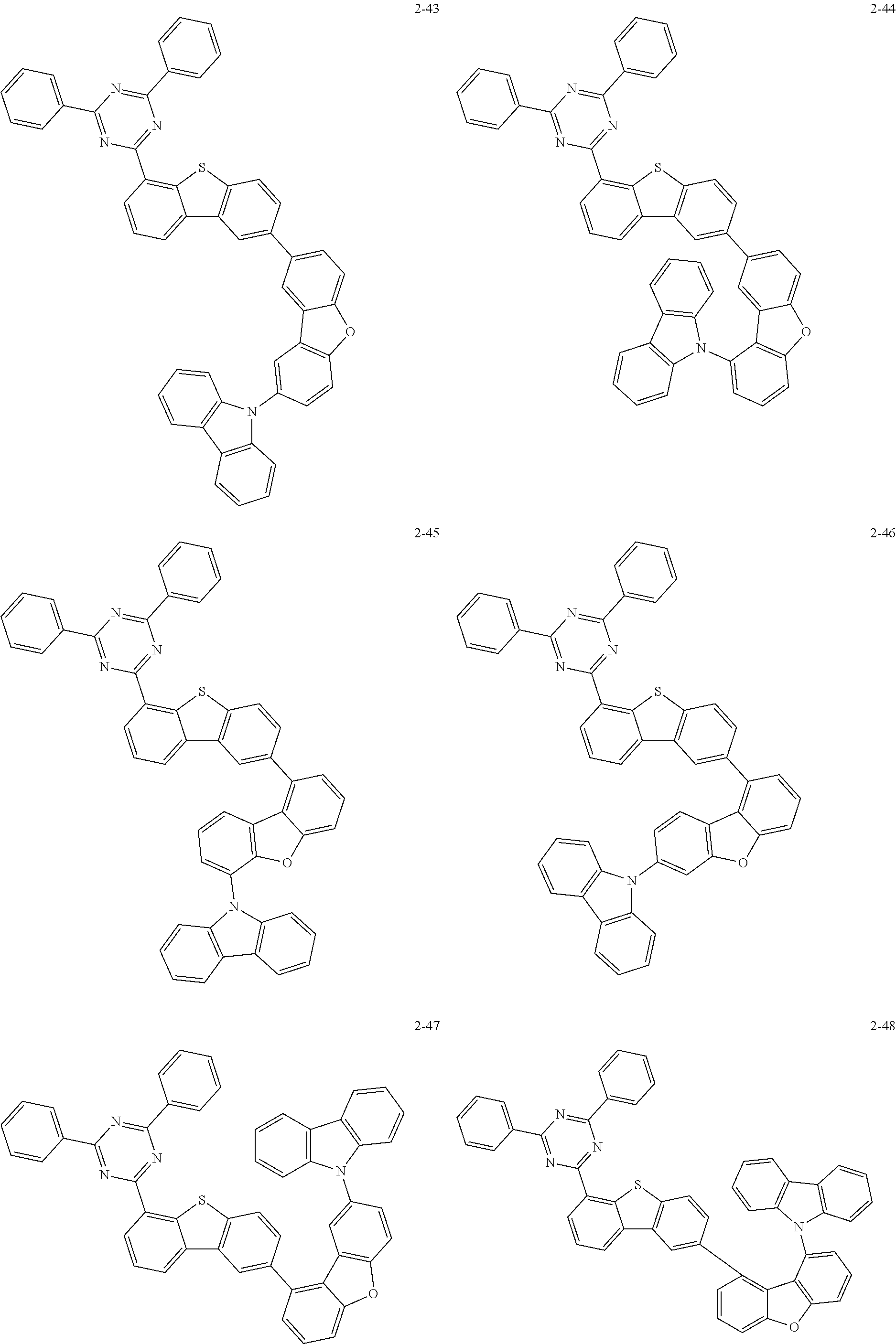

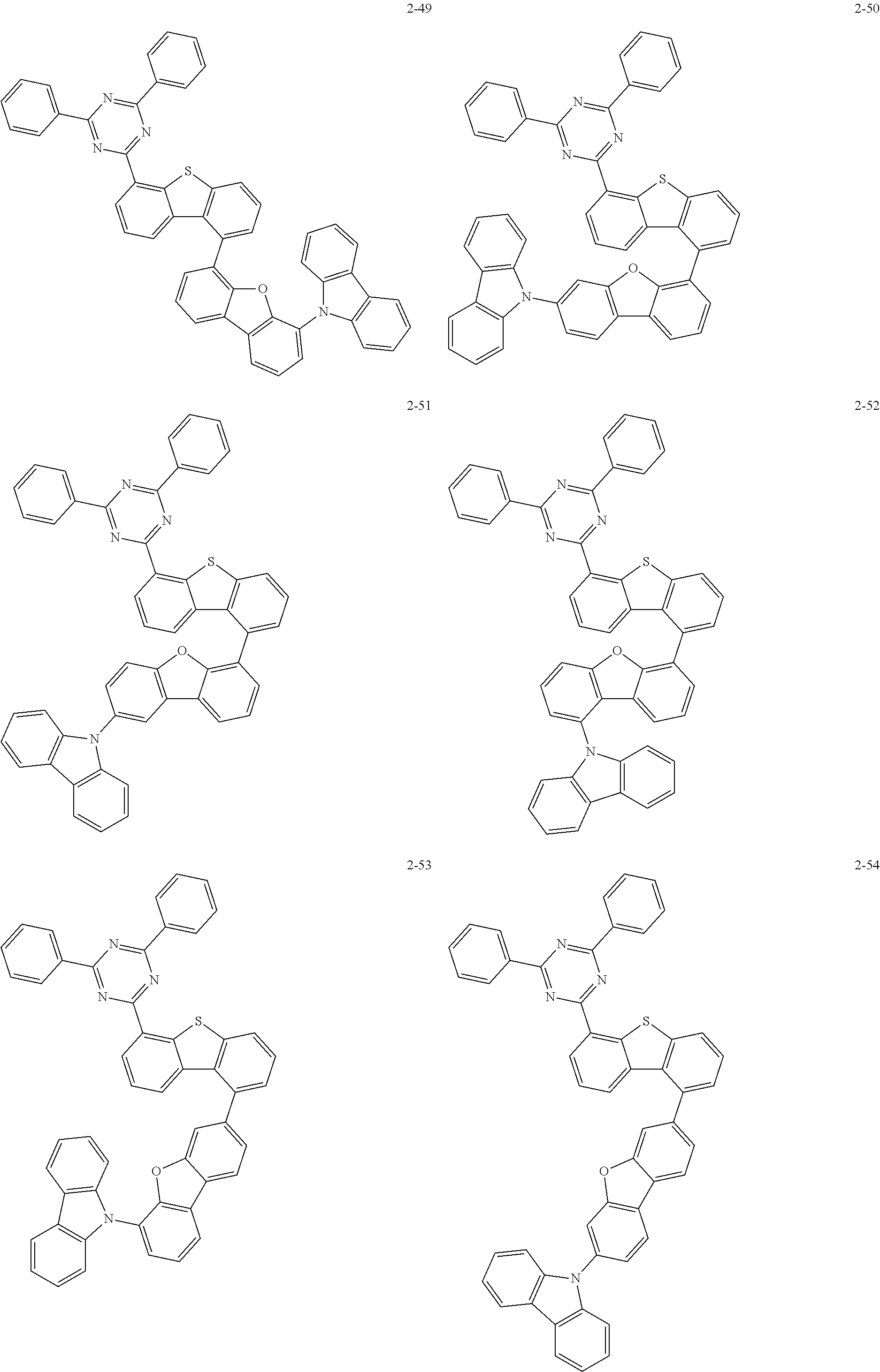

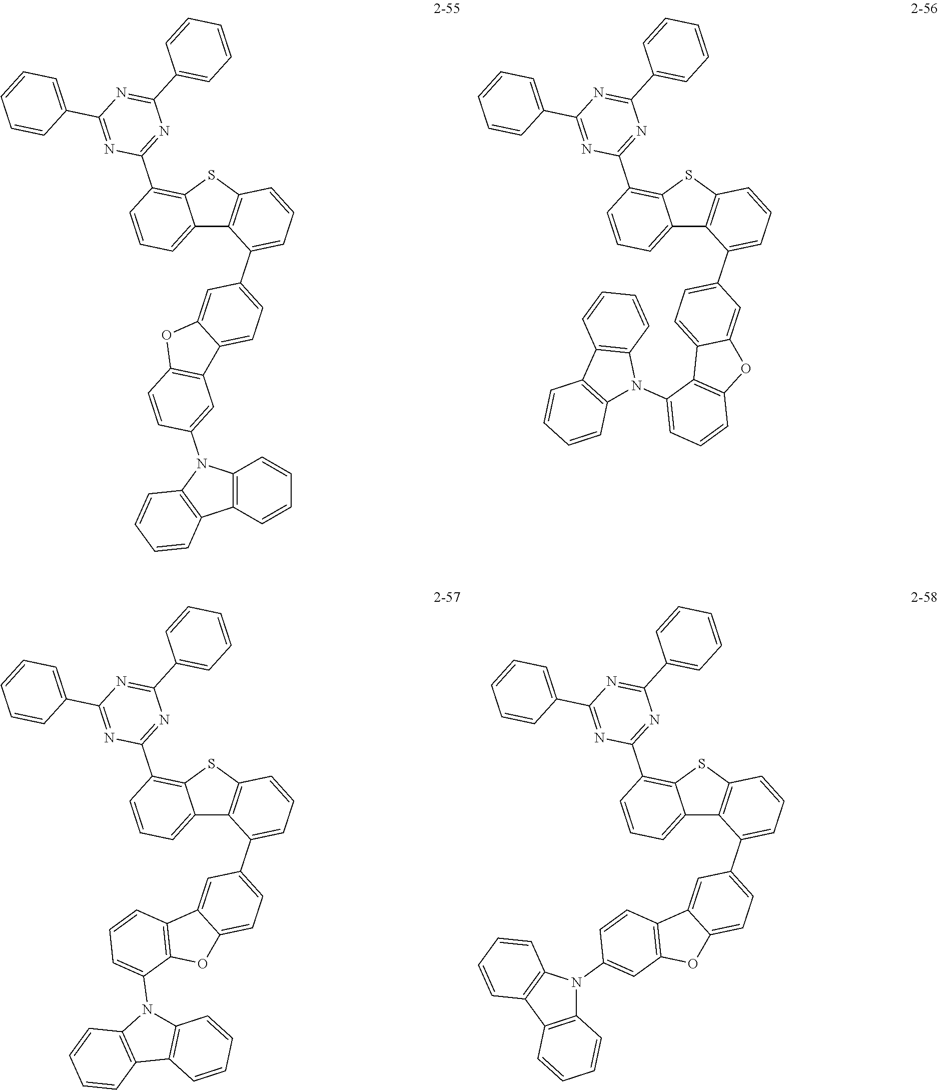 [SMILES: c1ccc(-c2nc(-c3ccccc3)nc(-c3cccc4c3sc3cccc(-c5ccc6c(c5)oc5ccc(-n7c8ccccc8c8ccccc87)cc56)c34)n2)cc1 ; c1ccc(-c2nc(-c3ccccc3)nc(-c3cccc4c3sc3cccc(-c5ccc6c(c5)oc5cccc(-n7c8ccccc8c8ccccc87)c56)c34)n2)cc1 ; c1ccc(-c2nc(-c3ccccc3)nc(-c3cccc4c3sc3cccc(-c5ccc6oc7c(-n8c9ccccc9c9ccccc98)cccc7c6c5)c34)n2)cc1 ; c1ccc(-c2nc(-c3ccccc3)nc(-c3cccc4c3sc3cccc(-c5ccc6oc7cc(-n8c9ccccc9c9ccccc98)ccc7c6c5)c34)n2)cc1]

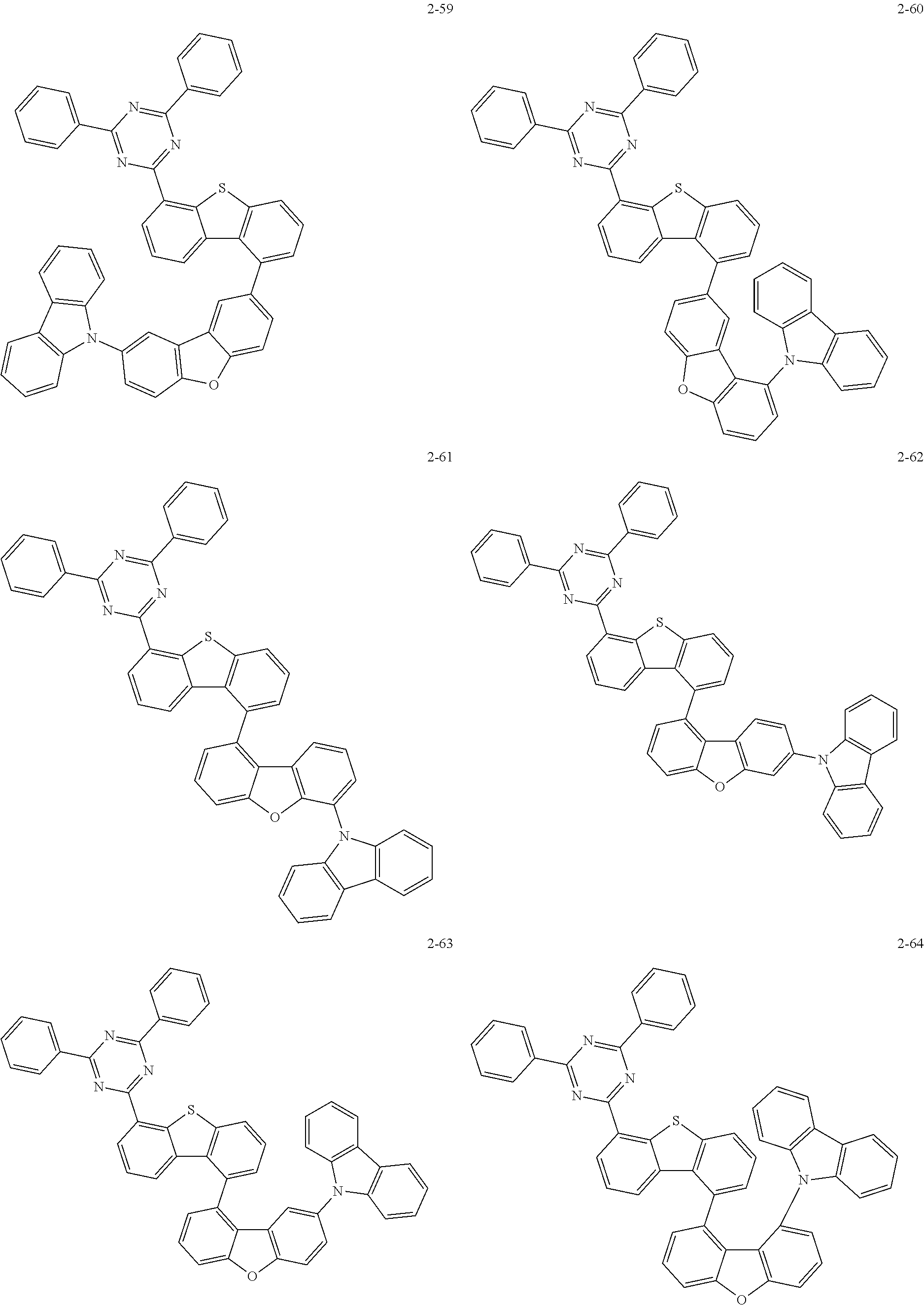

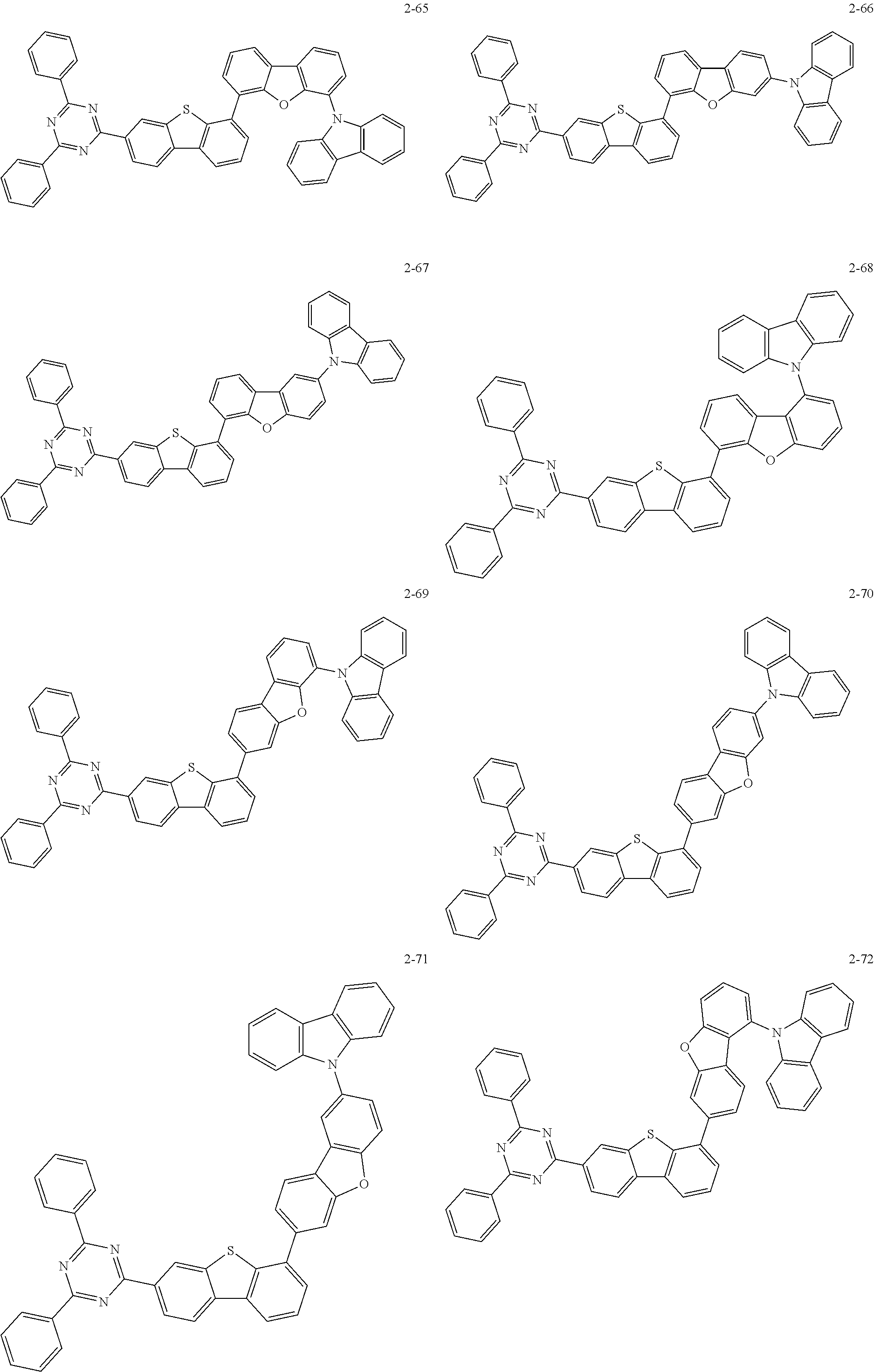

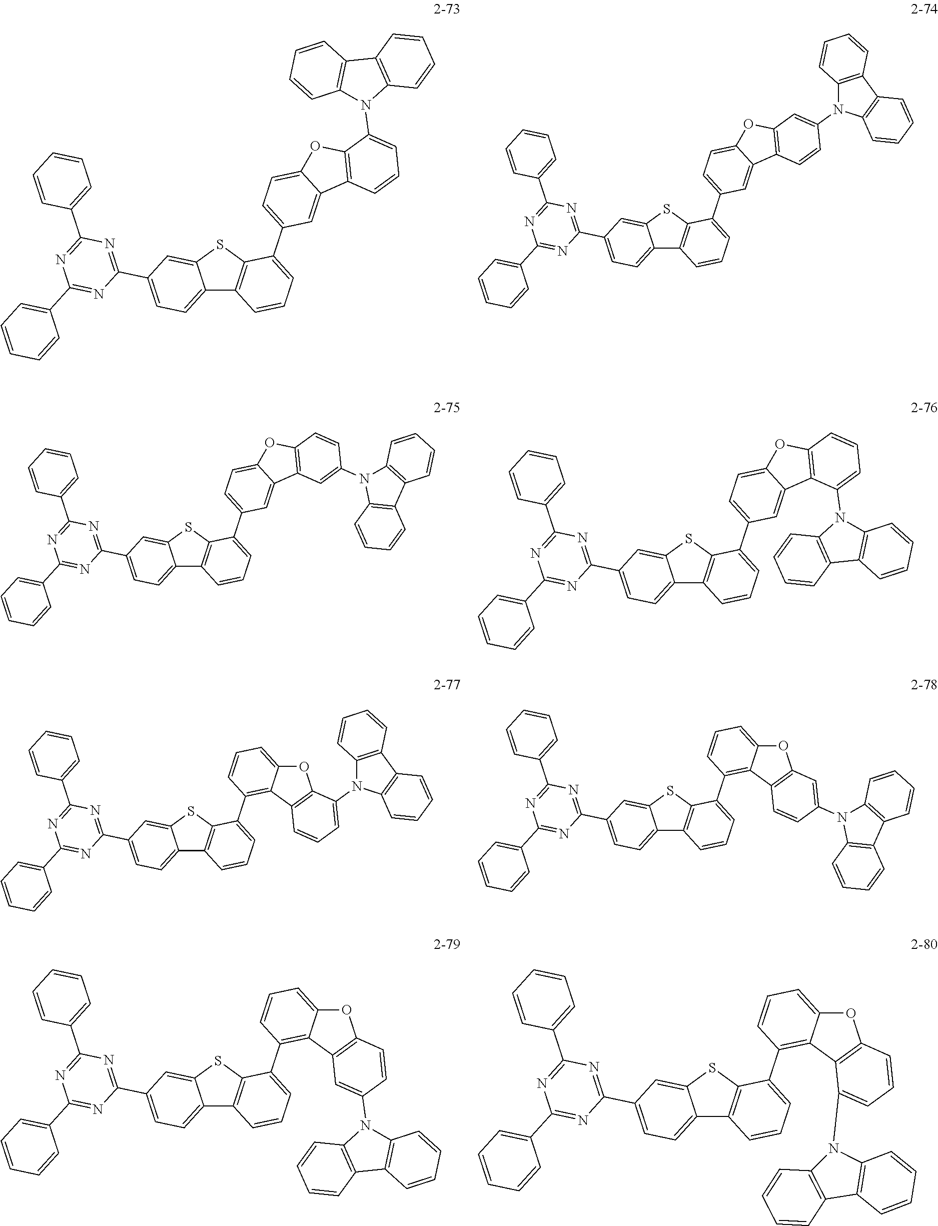

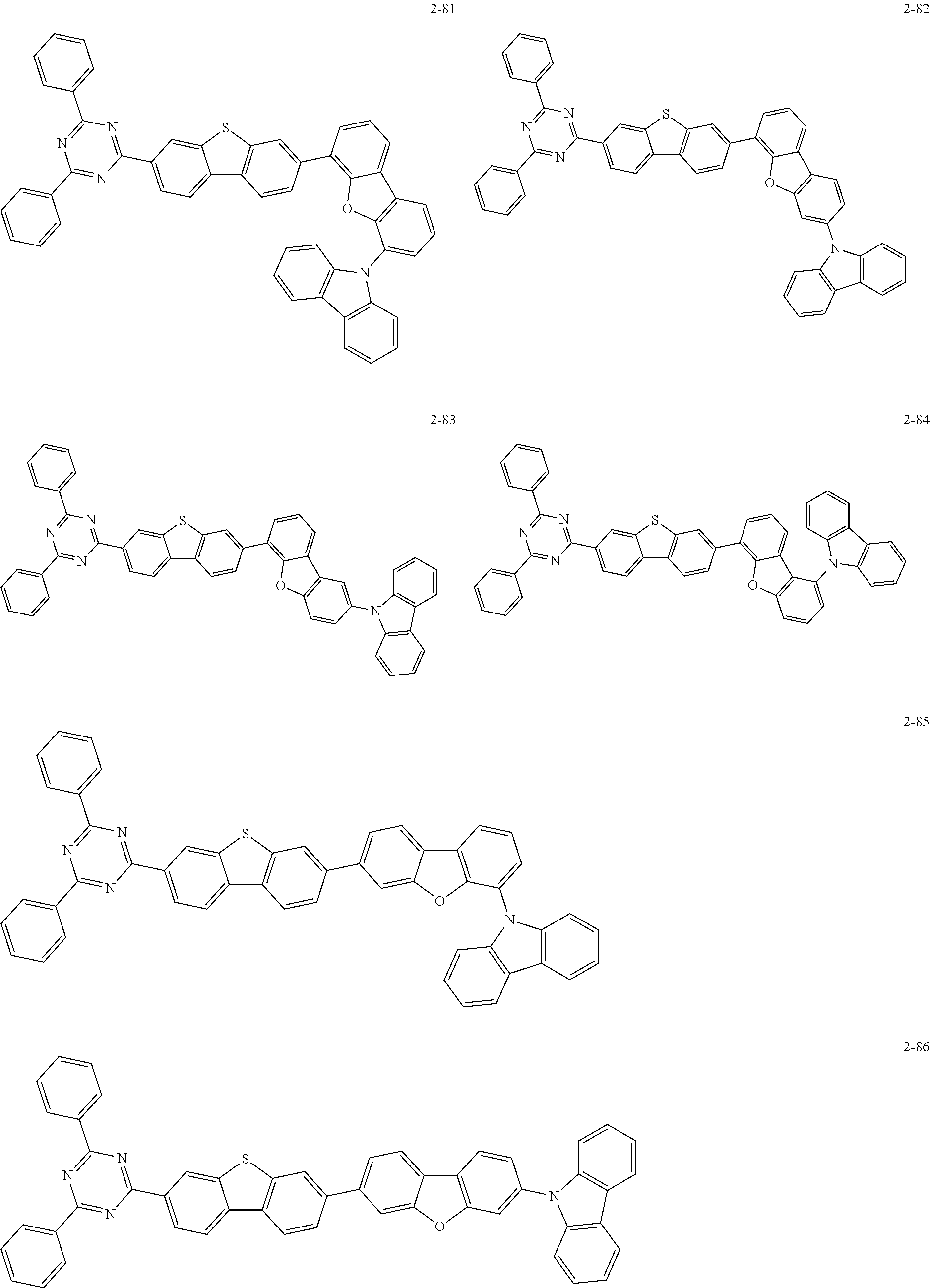

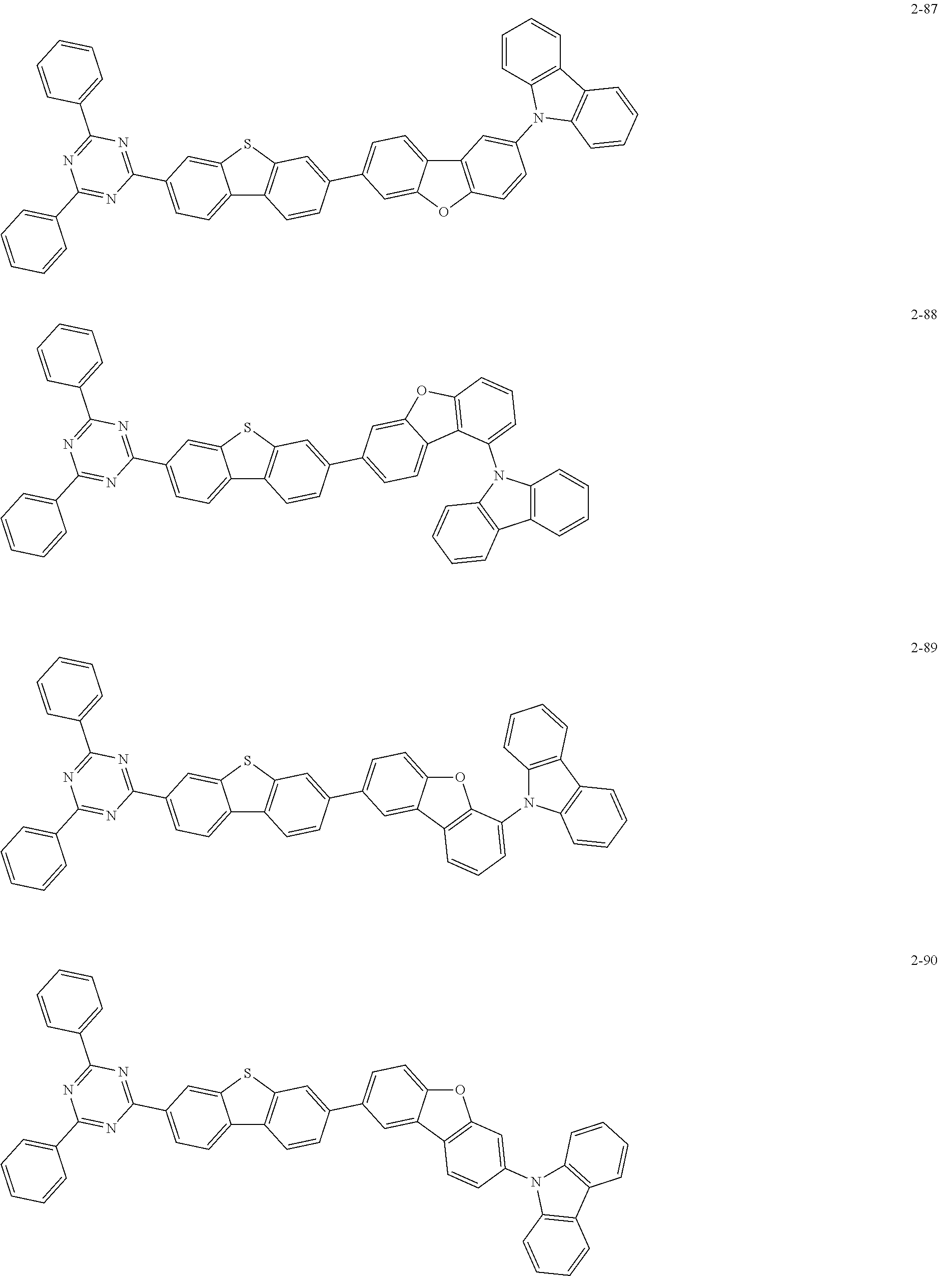 [SMILES: c1ccc(-c2nc(-c3ccccc3)nc(-c3ccc4c(c3)sc3cc(-c5ccc6c(c5)oc5ccc(-n7c8ccccc8c8ccccc87)cc56)ccc34)n2)cc1 ; c1ccc(-c2nc(-c3ccccc3)nc(-c3ccc4c(c3)sc3cc(-c5ccc6c(c5)oc5cccc(-n7c8ccccc8c8ccccc87)c56)ccc34)n2)cc1 ; c1ccc(-c2nc(-c3ccccc3)nc(-c3ccc4c(c3)sc3cc(-c5ccc6oc7c(-n8c9ccccc9c9ccccc98)cccc7c6c5)ccc34)n2)cc1 ; c1ccc(-c2nc(-c3ccccc3)nc(-c3ccc4c(c3)sc3cc(-c5ccc6oc7cc(-n8c9ccccc9c9ccccc98)ccc7c6c5)ccc34)n2)cc1]

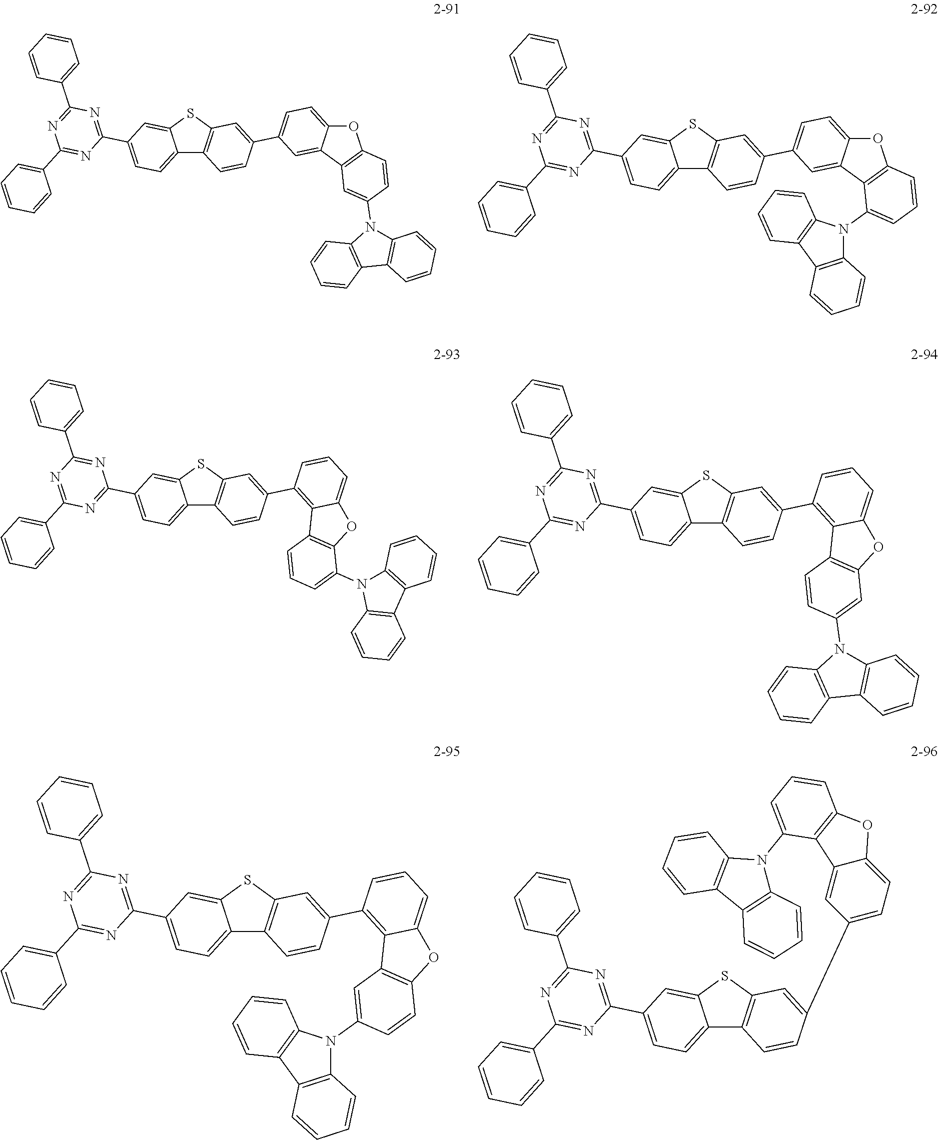

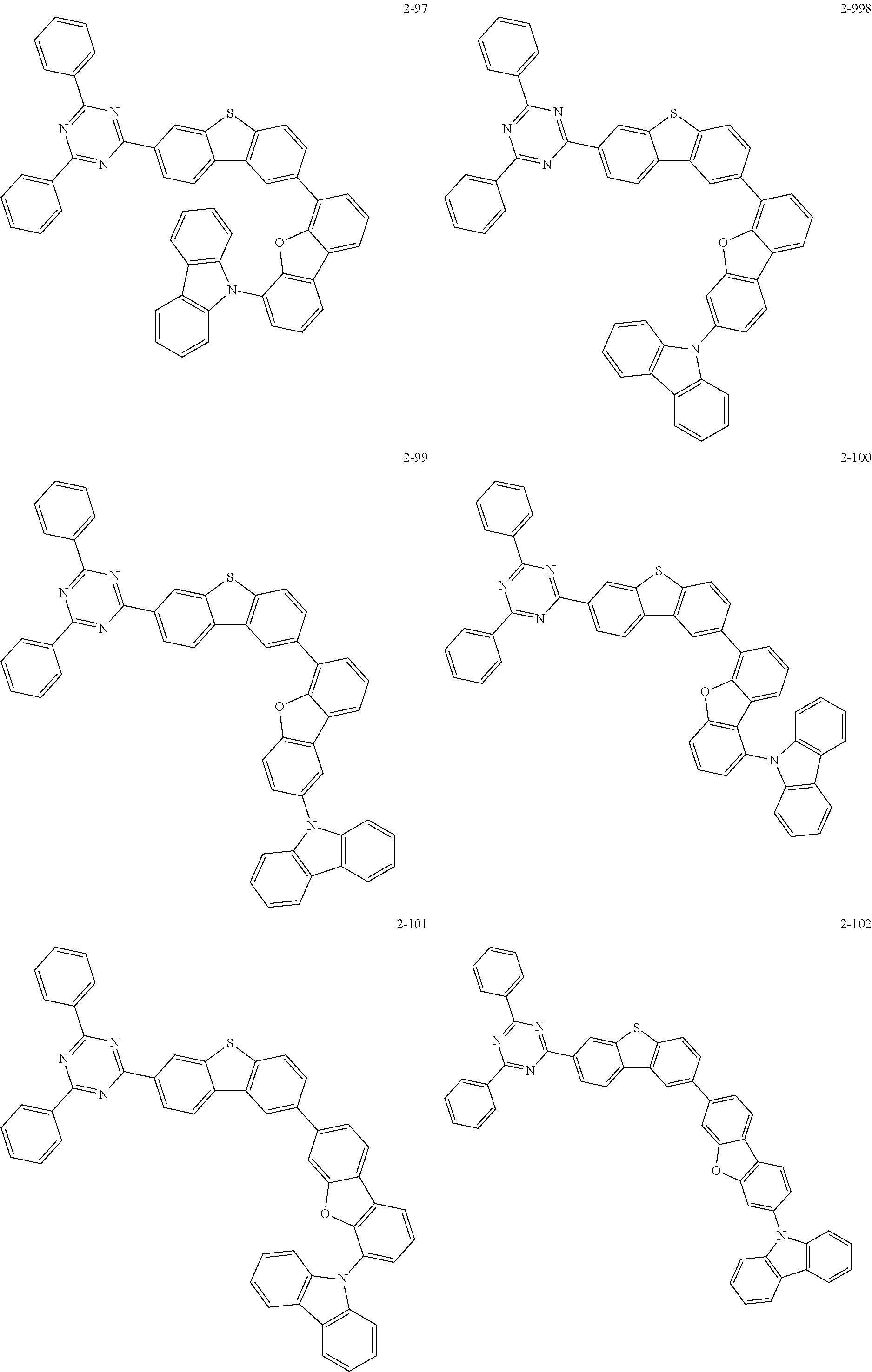

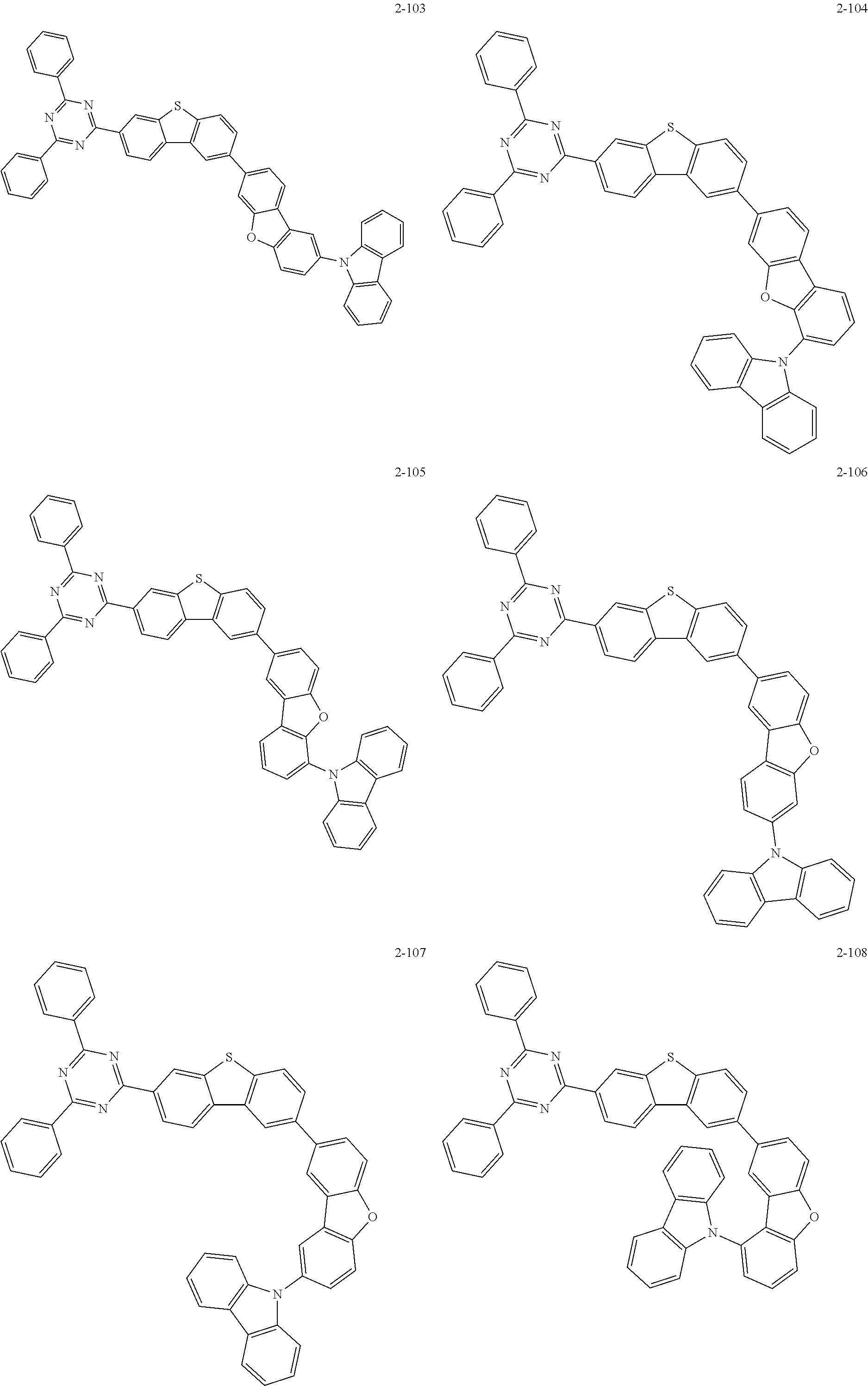

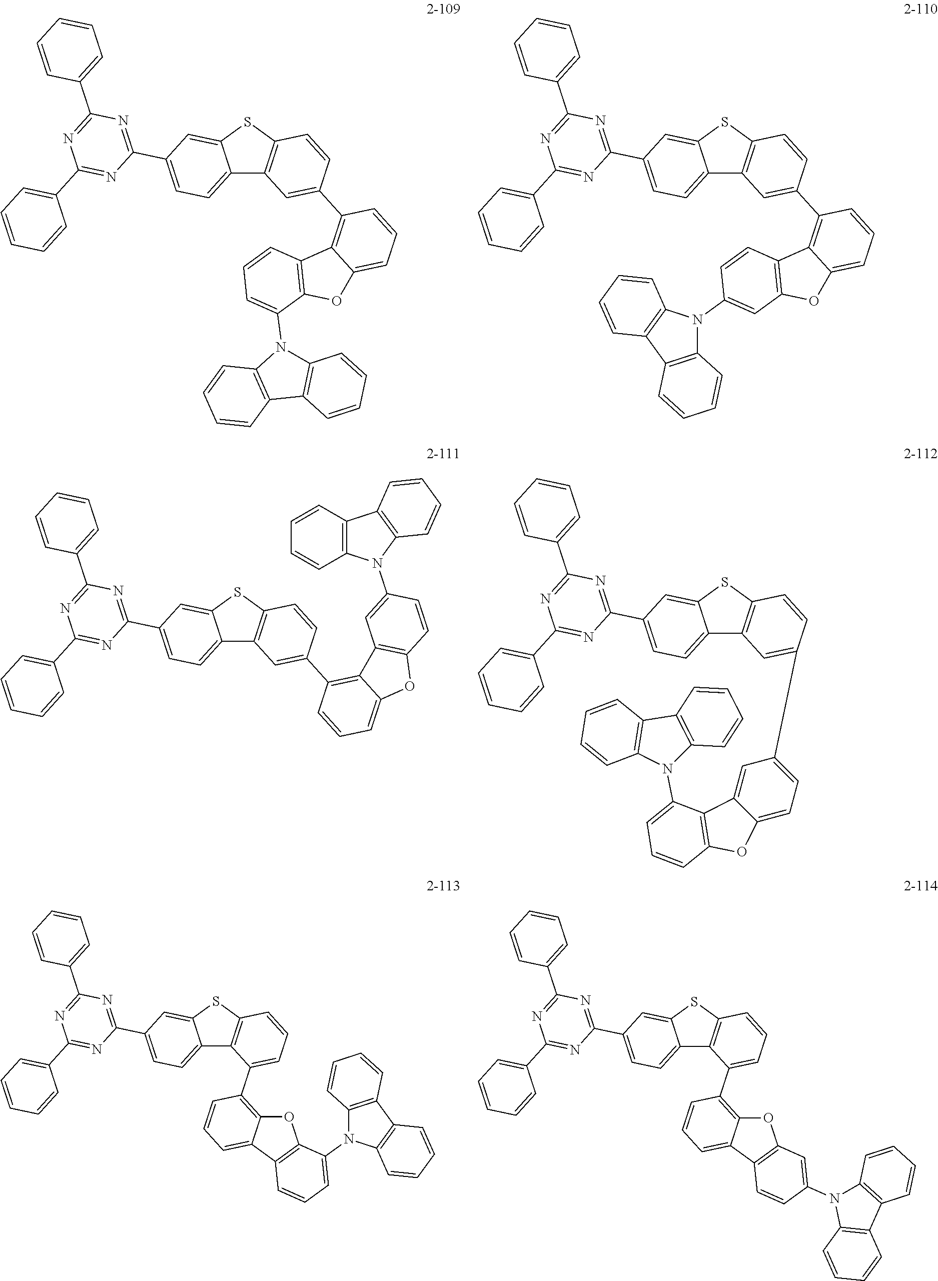

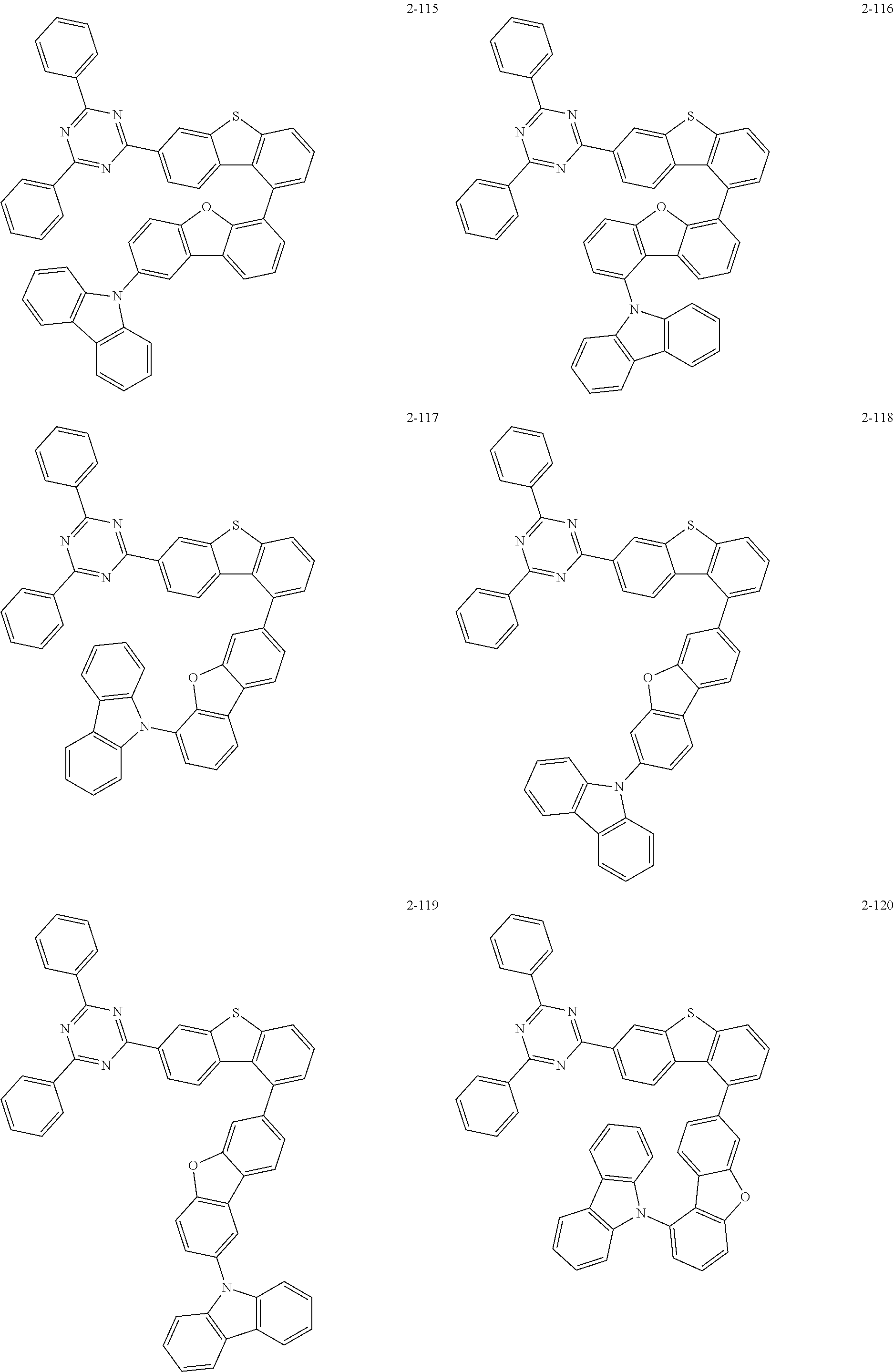

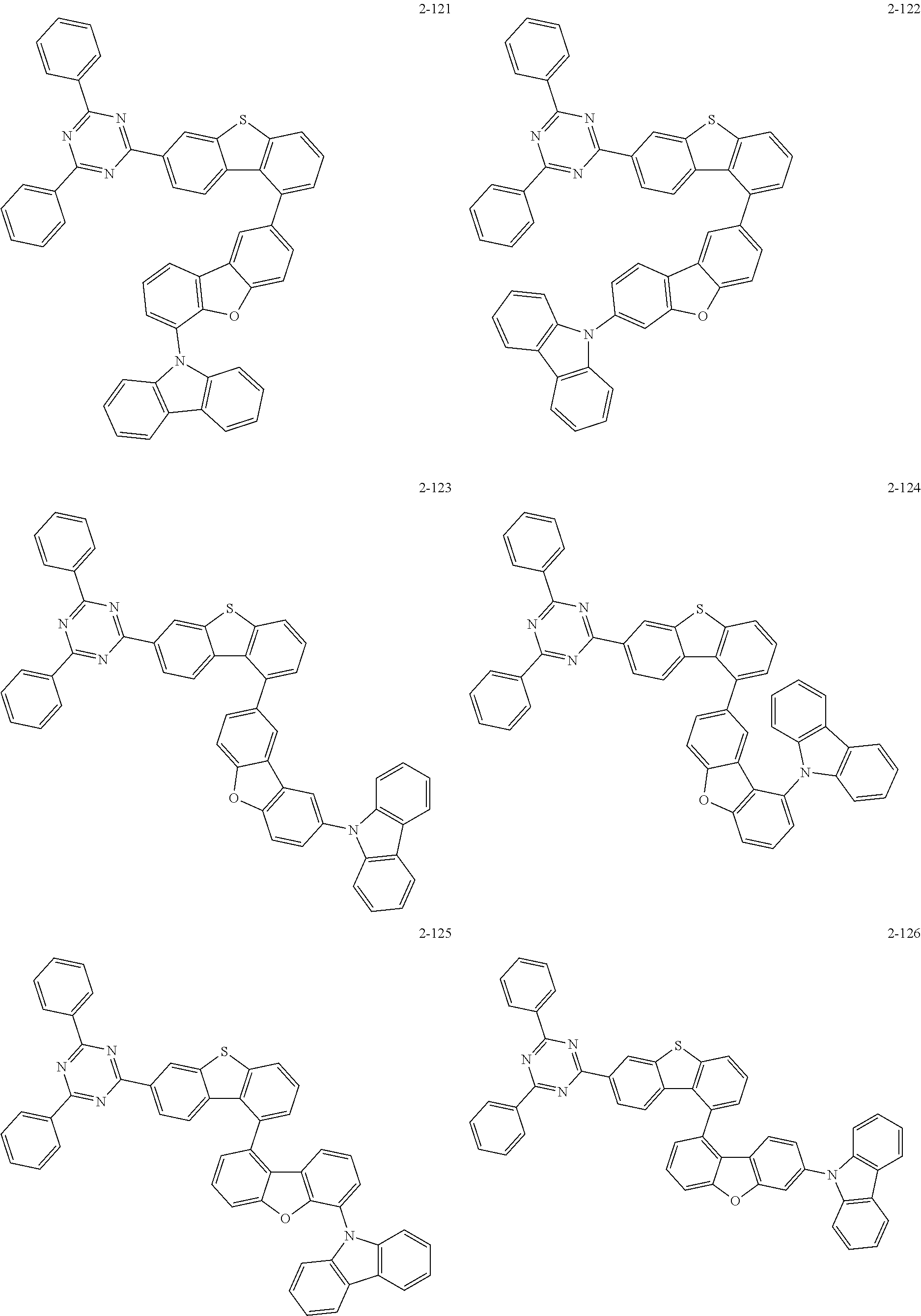

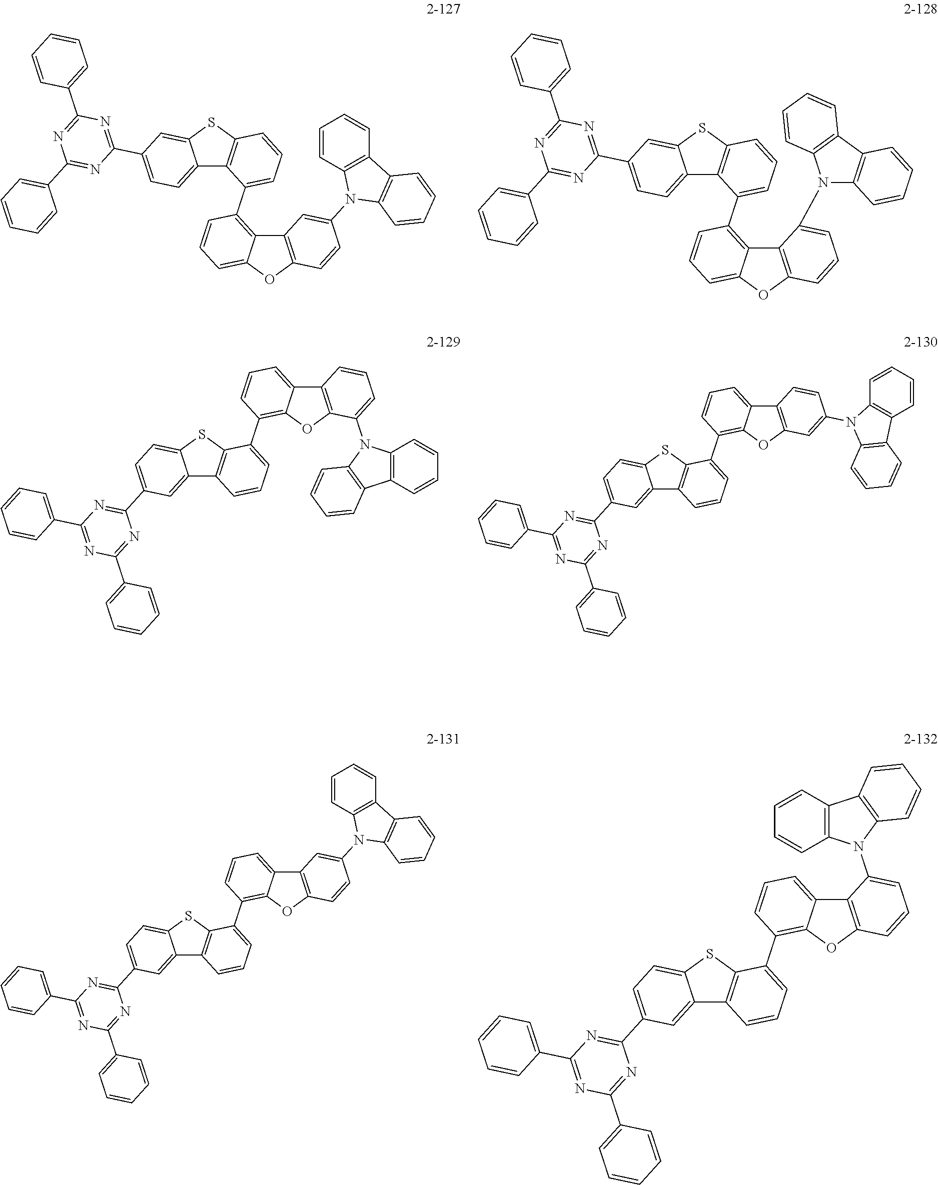

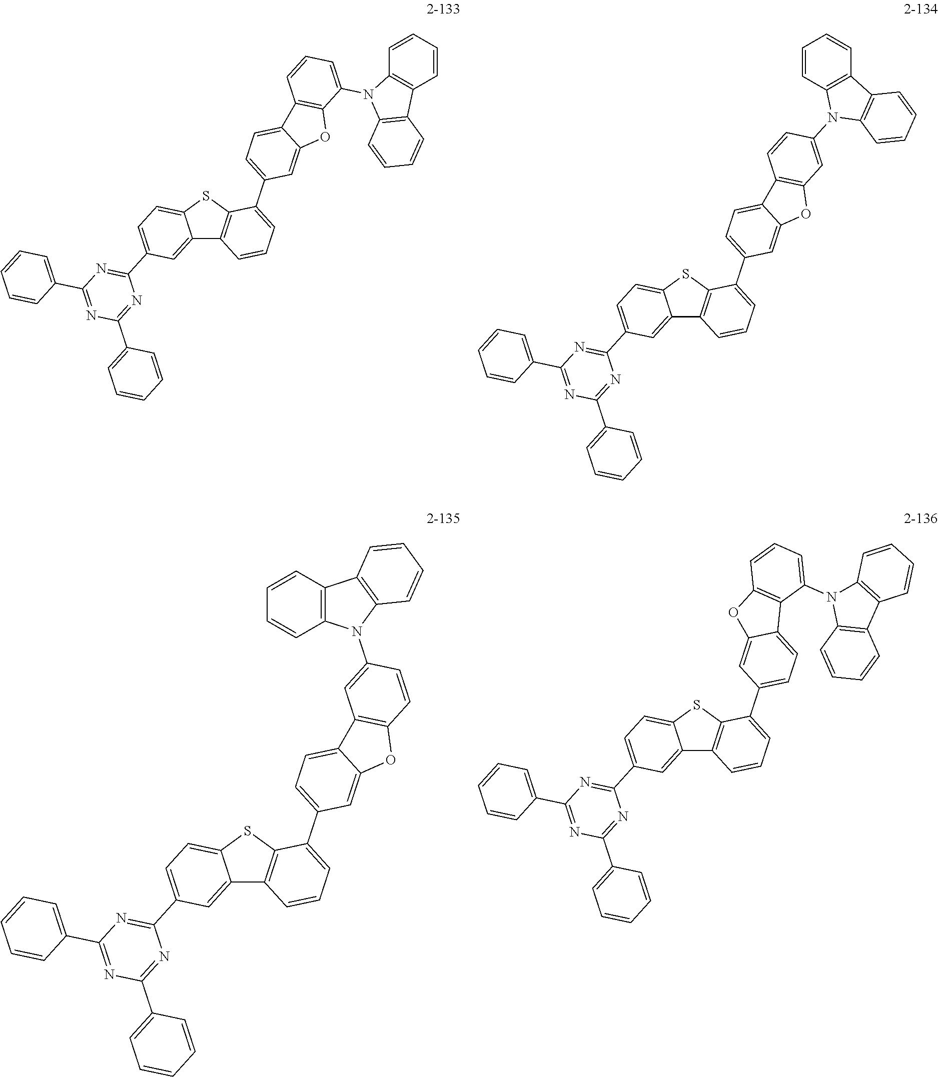 [SMILES: c1ccc(-c2nc(-c3ccccc3)nc(-c3ccc4sc5c(-c6ccc7c(c6)oc6c(-n8c9ccccc9c9ccccc98)cccc67)cccc5c4c3)n2)cc1 ; c1ccc(-c2nc(-c3ccccc3)nc(-c3ccc4sc5c(-c6ccc7c(c6)oc6cc(-n8c9ccccc9c9ccccc98)ccc67)cccc5c4c3)n2)cc1 ; c1ccc(-c2nc(-c3ccccc3)nc(-c3ccc4sc5c(-c6ccc7c(c6)oc6ccc(-n8c9ccccc9c9ccccc98)cc67)cccc5c4c3)n2)cc1 ; c1ccc(-c2nc(-c3ccccc3)nc(-c3ccc4sc5c(-c6ccc7c(c6)oc6cccc(-n8c9ccccc9c9ccccc98)c67)cccc5c4c3)n2)cc1]

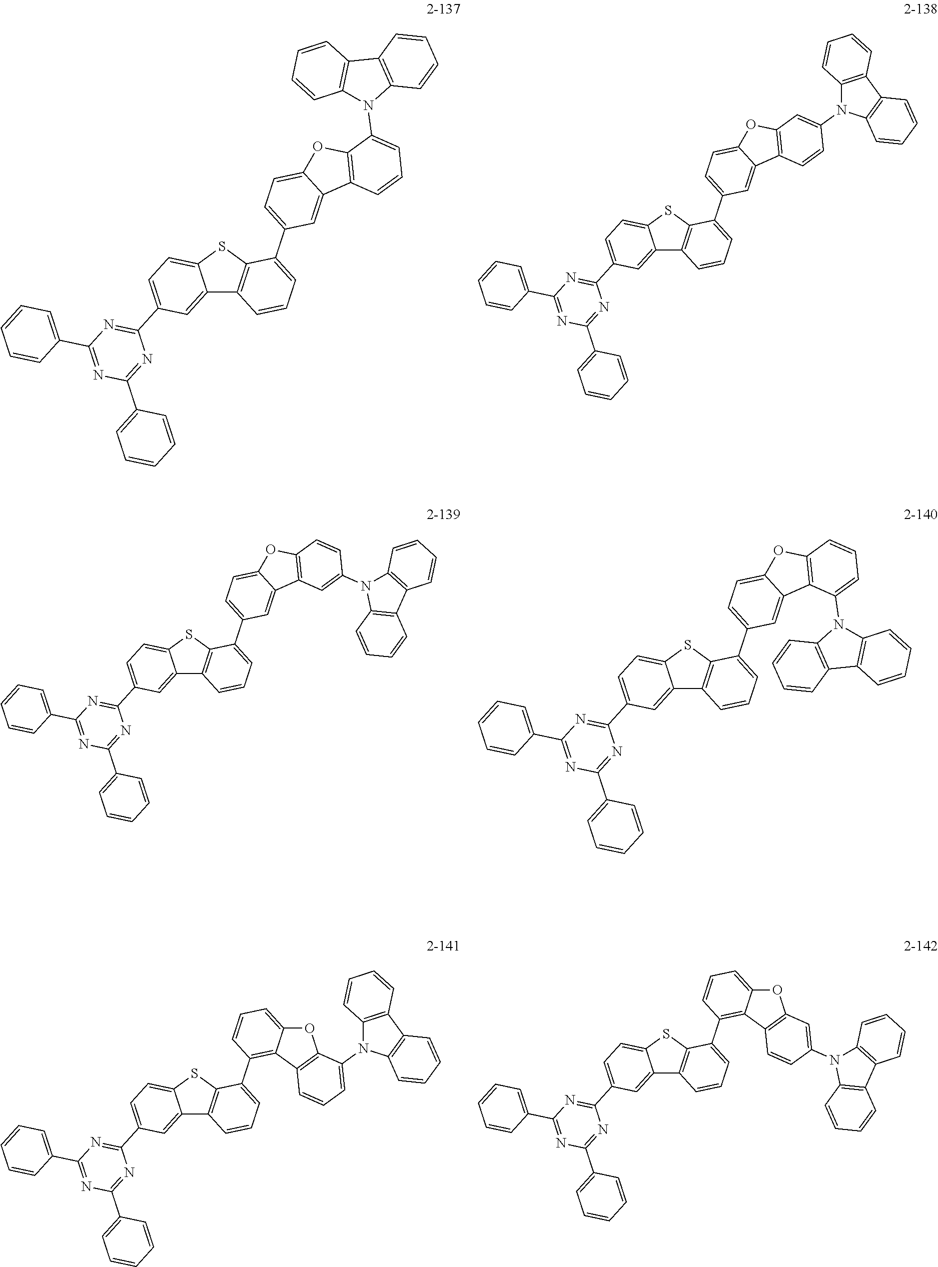

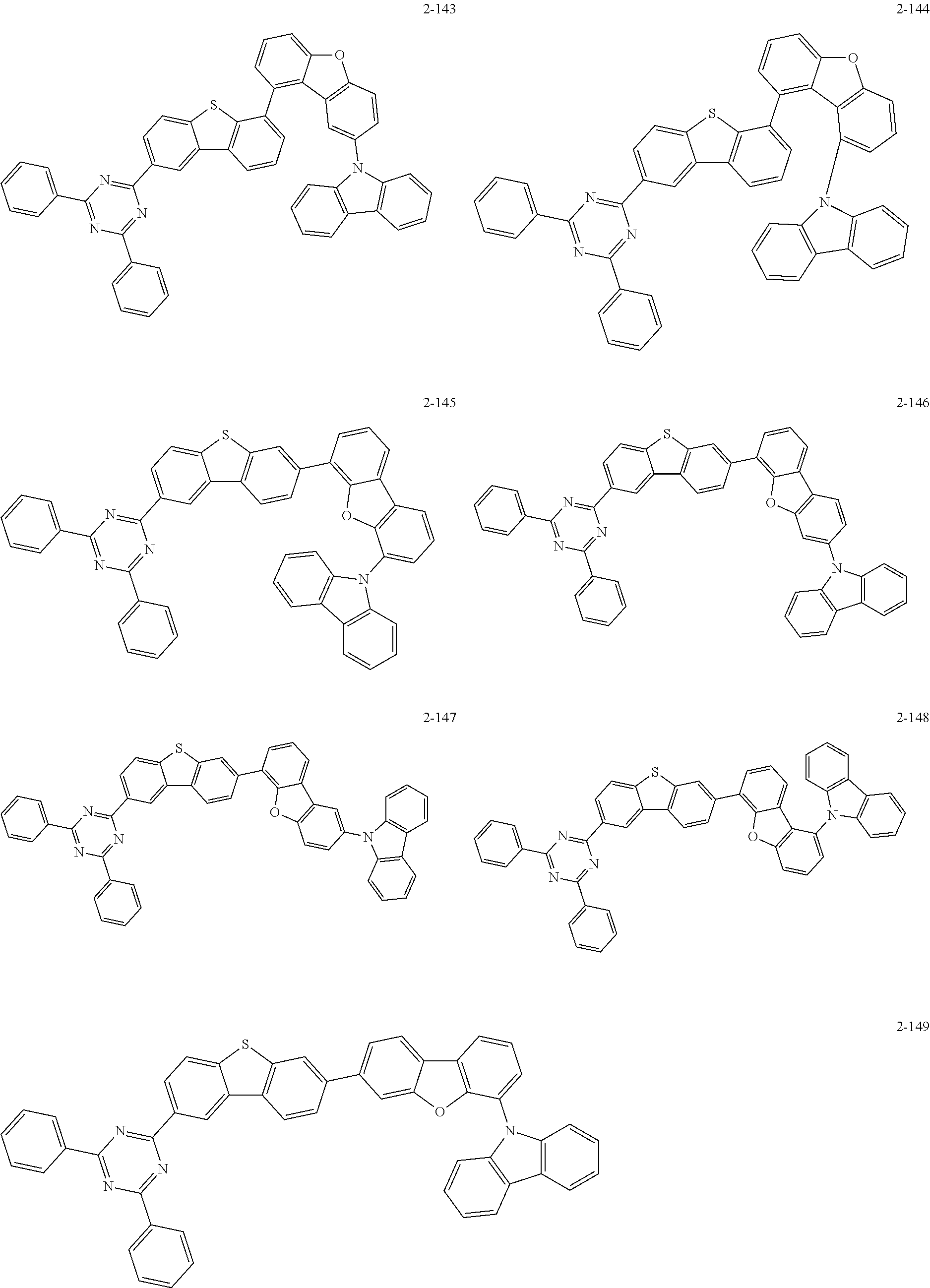

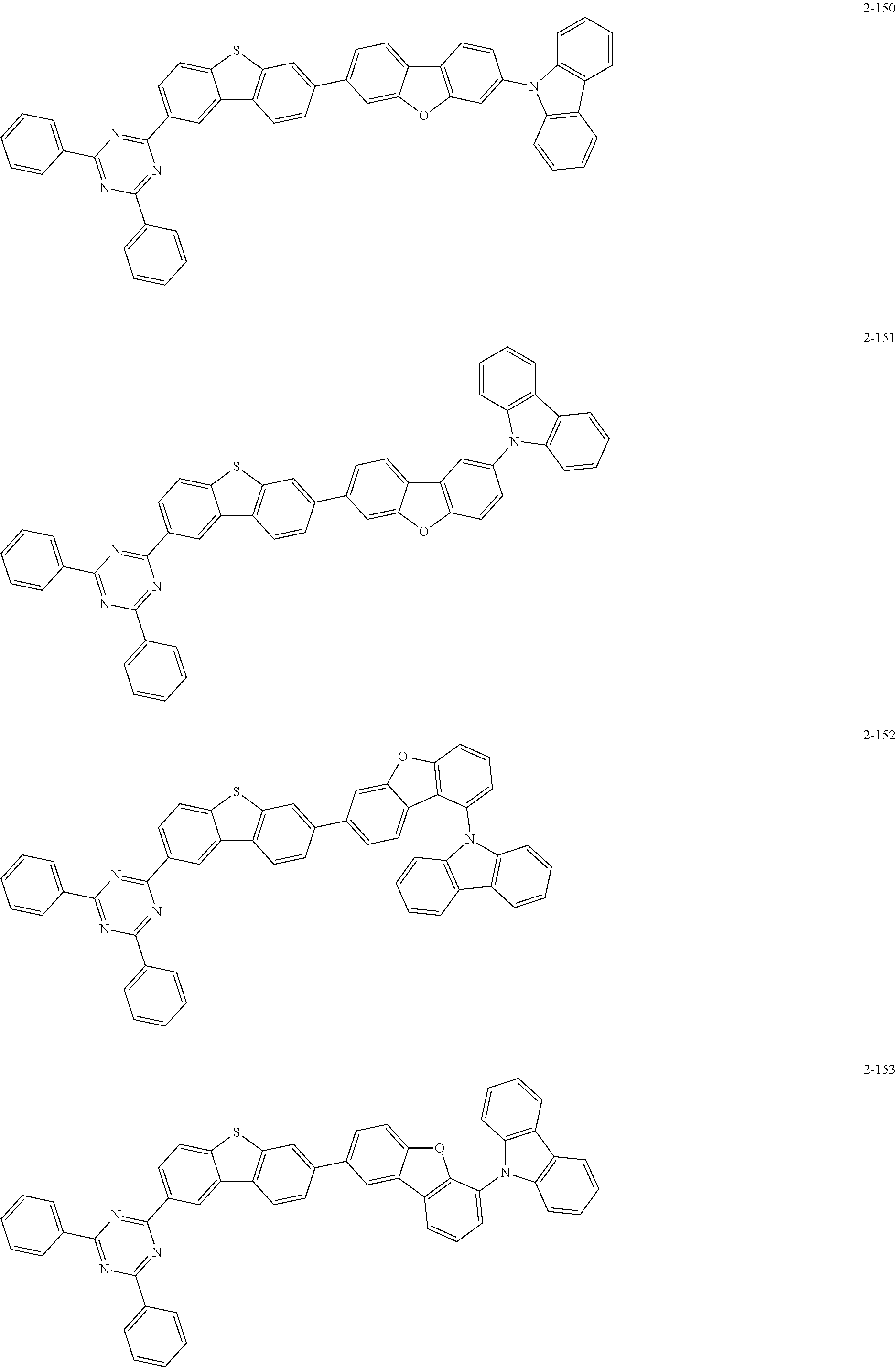 [SMILES: c1ccc(-c2nc(-c3ccccc3)nc(-c3ccc4sc5cc(-c6ccc7c(c6)oc6cc(-n8c9ccccc9c9ccccc98)ccc67)ccc5c4c3)n2)cc1 ; c1ccc(-c2nc(-c3ccccc3)nc(-c3ccc4sc5cc(-c6ccc7c(c6)oc6ccc(-n8c9ccccc9c9ccccc98)cc67)ccc5c4c3)n2)cc1 ; c1ccc(-c2nc(-c3ccccc3)nc(-c3ccc4sc5cc(-c6ccc7c(c6)oc6cccc(-n8c9ccccc9c9ccccc98)c67)ccc5c4c3)n2)cc1 ; c1ccc(-c2nc(-c3ccccc3)nc(-c3ccc4sc5cc(-c6ccc7oc8c(-n9c%10ccccc%10c%10ccccc%109)cccc8c7c6)ccc5c4c3)n2)cc1]

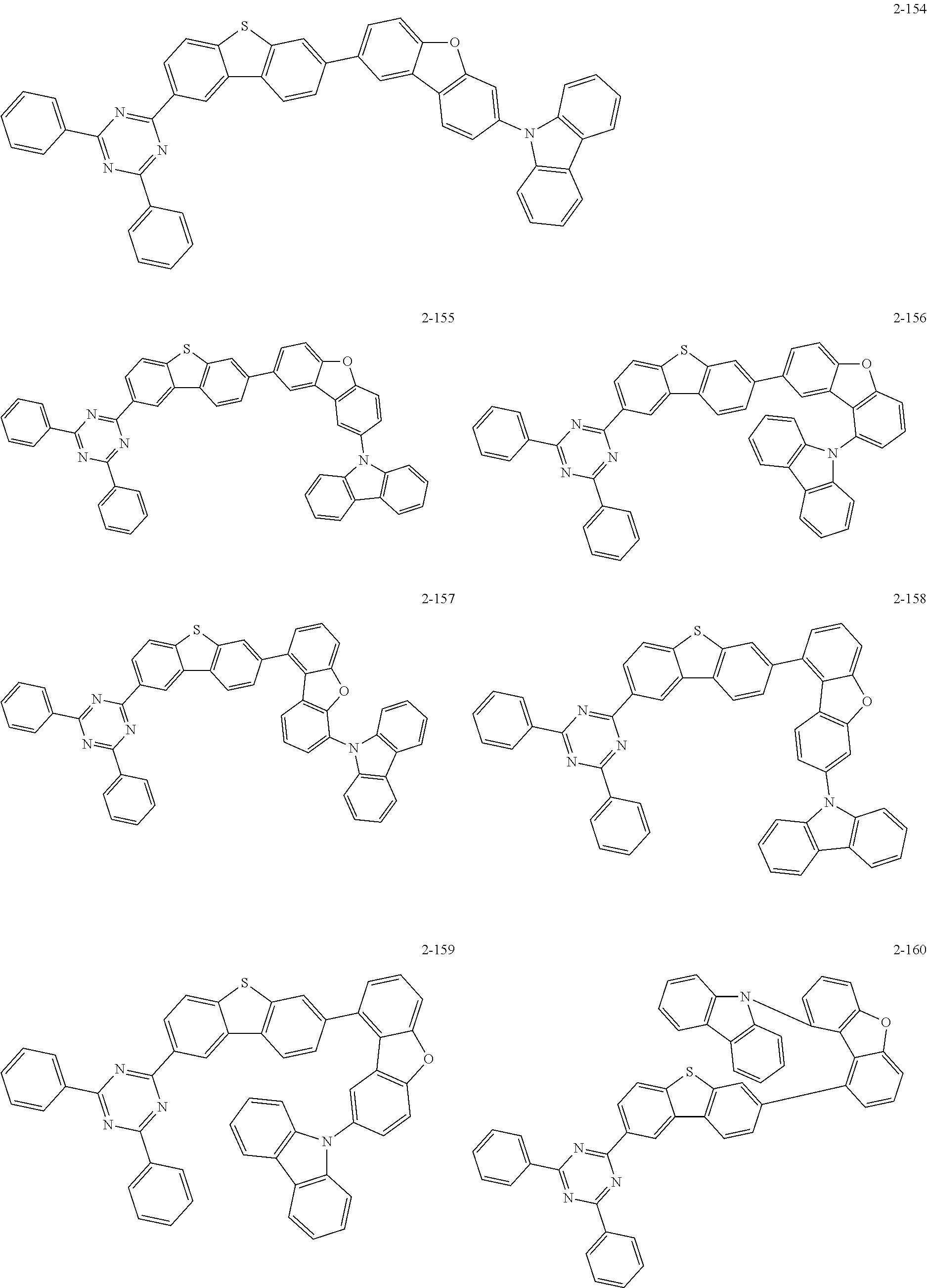

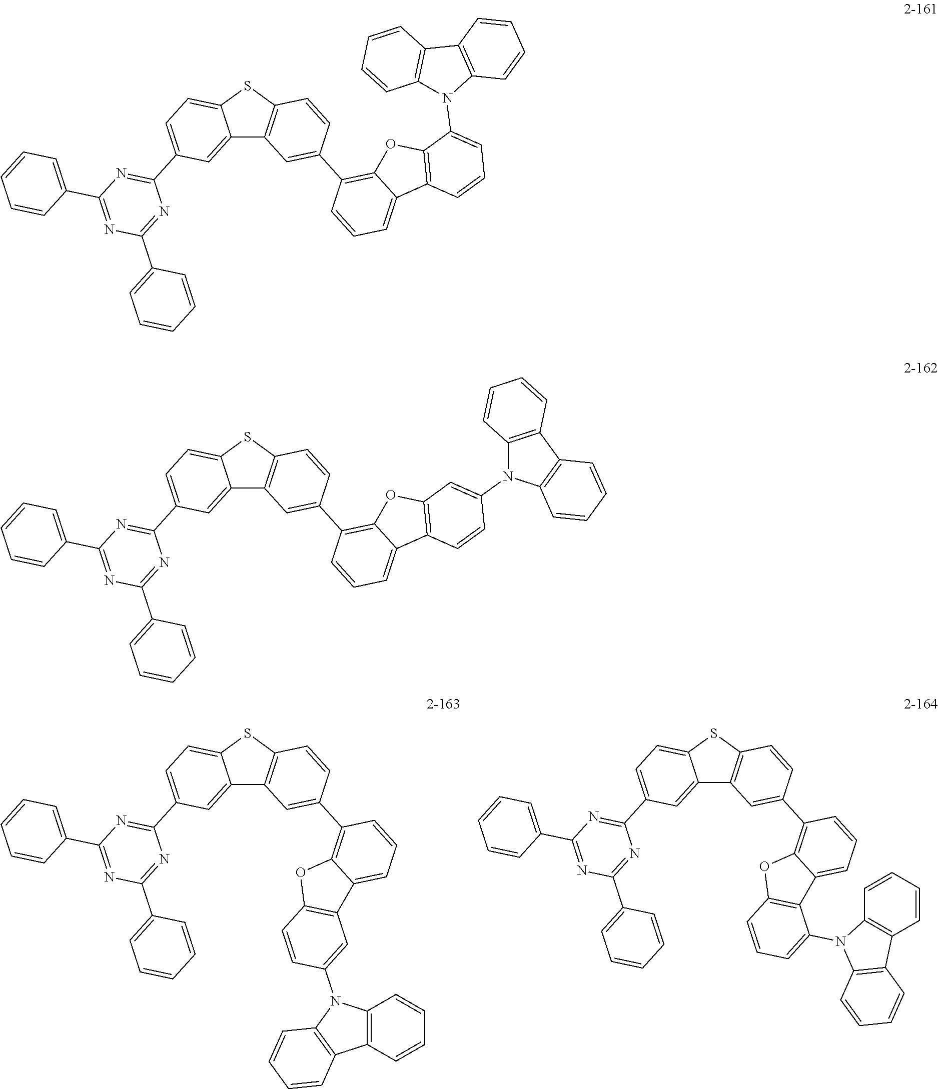 [SMILES: c1ccc(-c2nc(-c3ccccc3)nc(-c3ccc4sc5ccc(-c6cccc7c6oc6c(-n8c9ccccc9c9ccccc98)cccc67)cc5c4c3)n2)cc1 ; c1ccc(-c2nc(-c3ccccc3)nc(-c3ccc4sc5ccc(-c6cccc7c6oc6cc(-n8c9ccccc9c9ccccc98)ccc67)cc5c4c3)n2)cc1 ; c1ccc(-c2nc(-c3ccccc3)nc(-c3ccc4sc5ccc(-c6cccc7c6oc6ccc(-n8c9ccccc9c9ccccc98)cc67)cc5c4c3)n2)cc1 ; c1ccc(-c2nc(-c3ccccc3)nc(-c3ccc4sc5ccc(-c6cccc7c6oc6cccc(-n8c9ccccc9c9ccccc98)c67)cc5c4c3)n2)cc1]

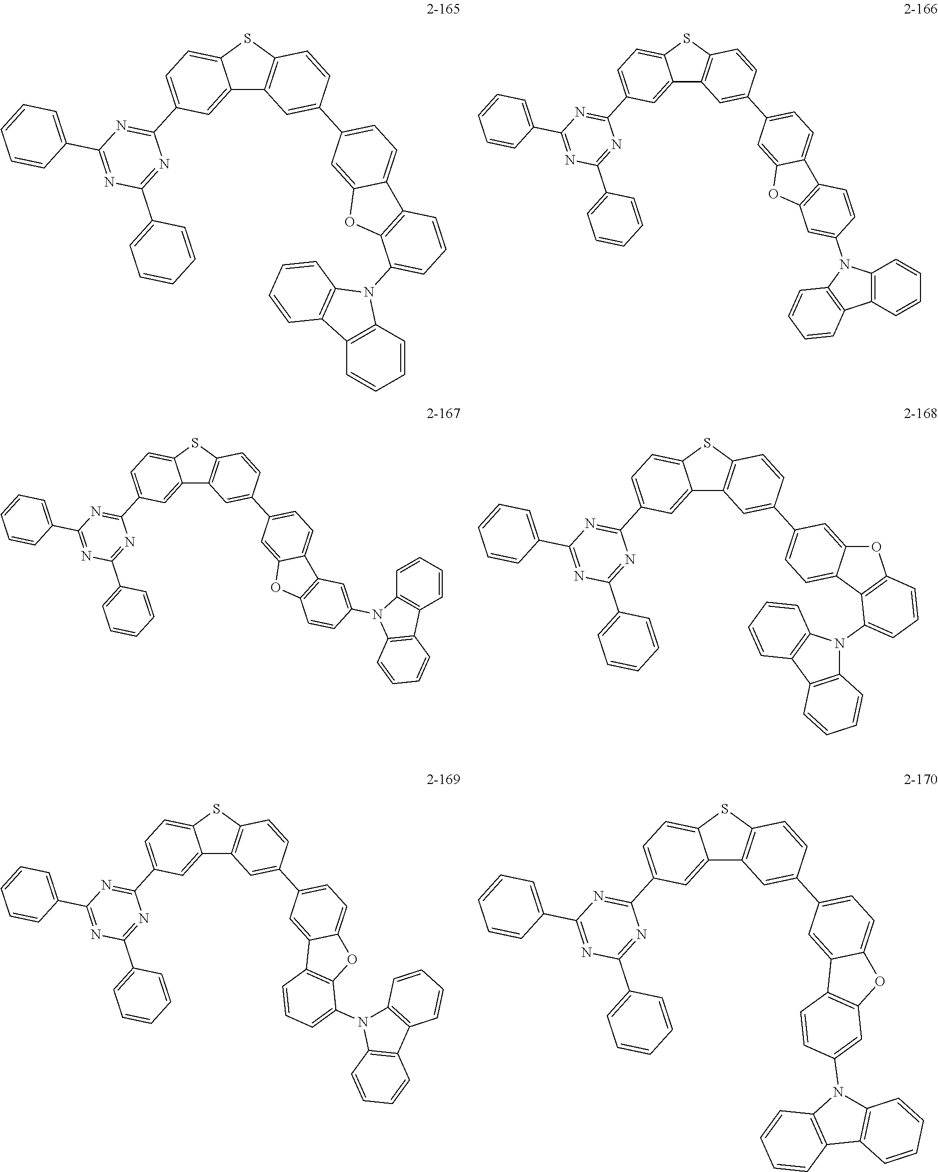

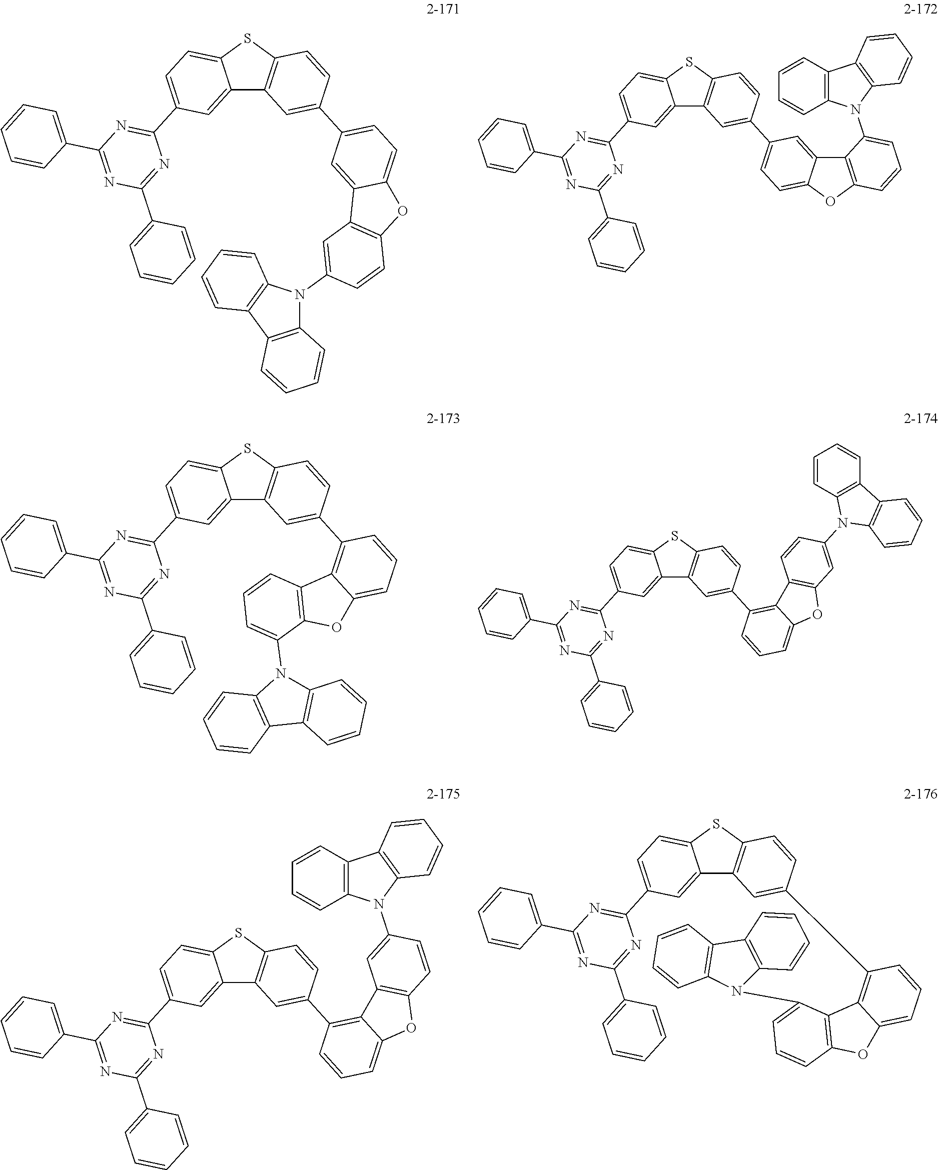

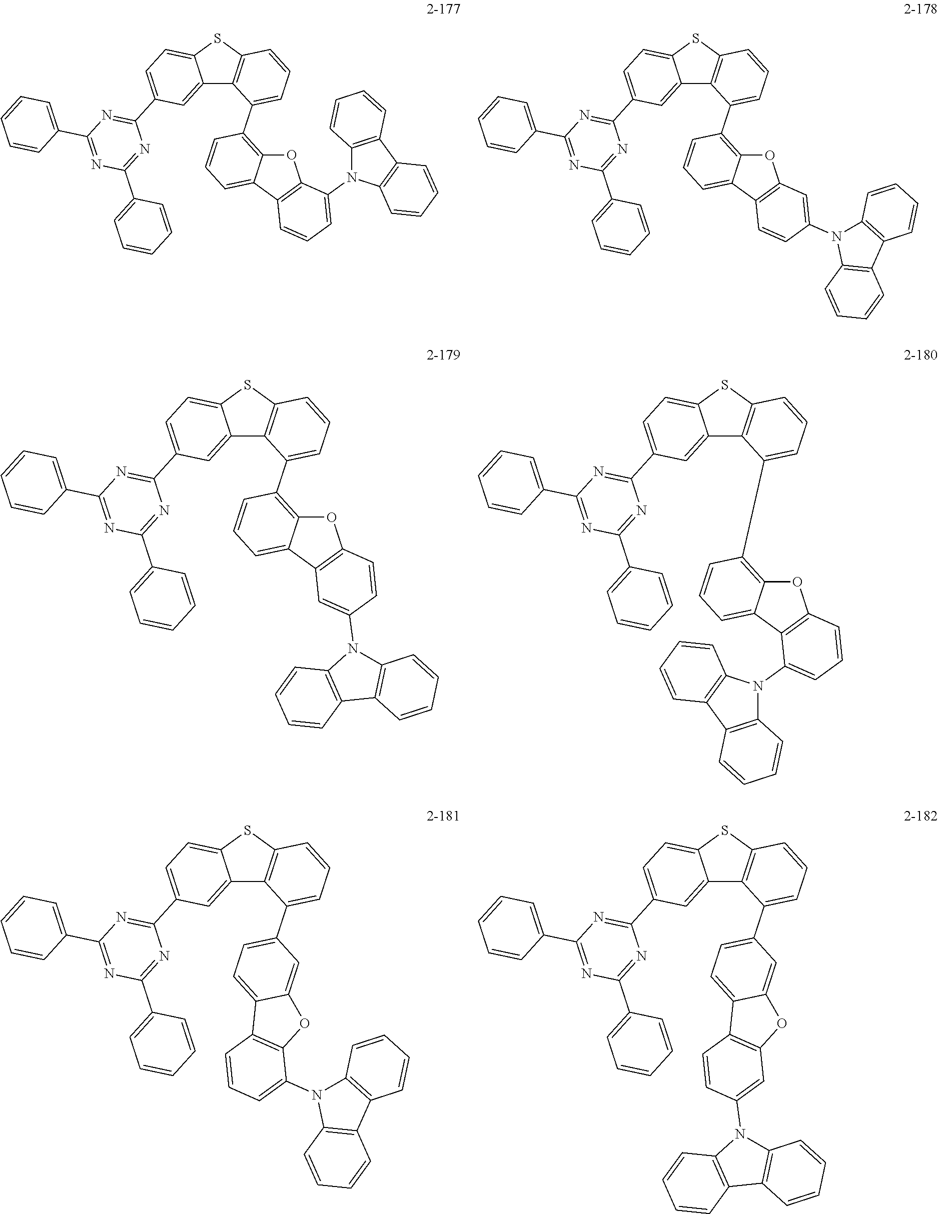

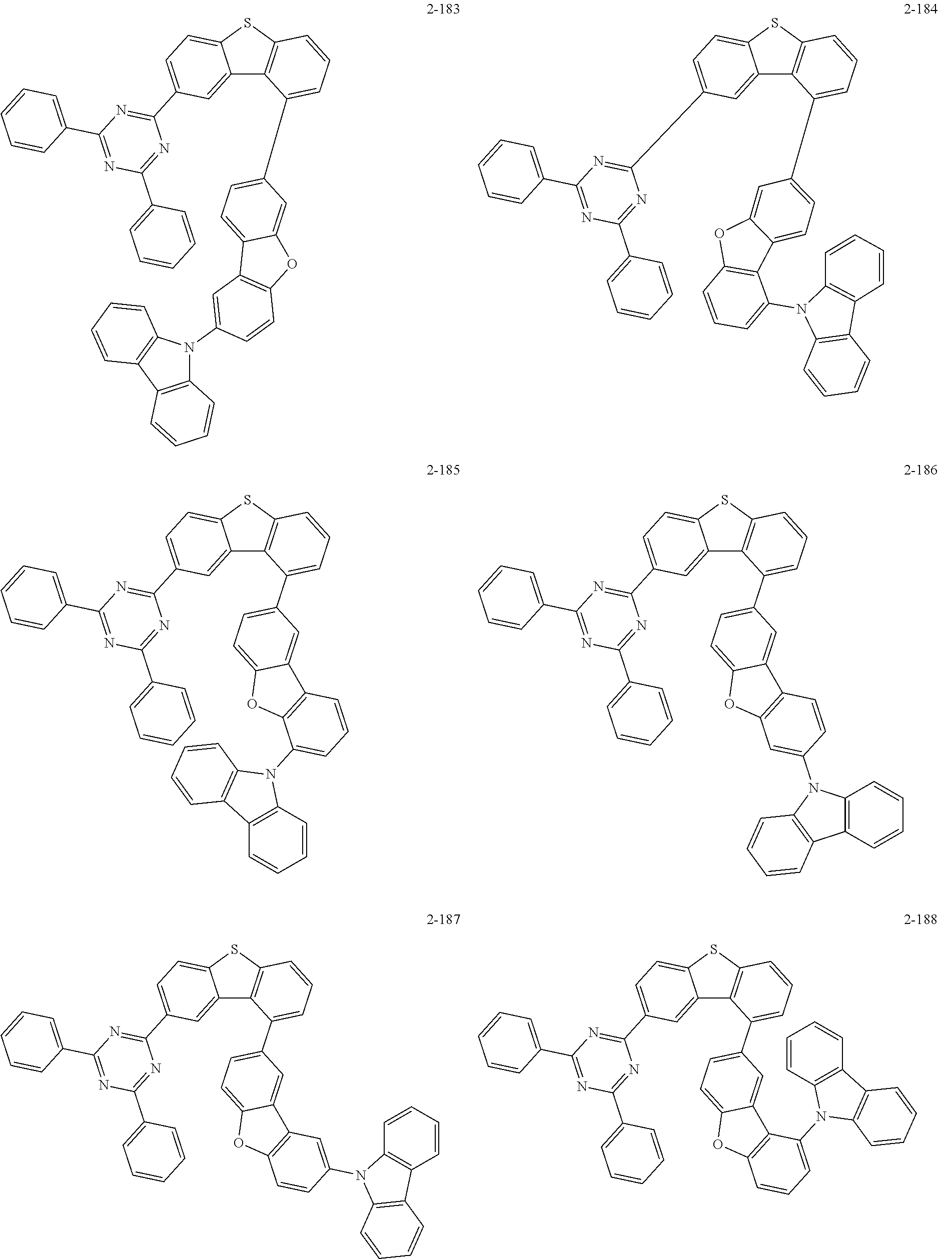

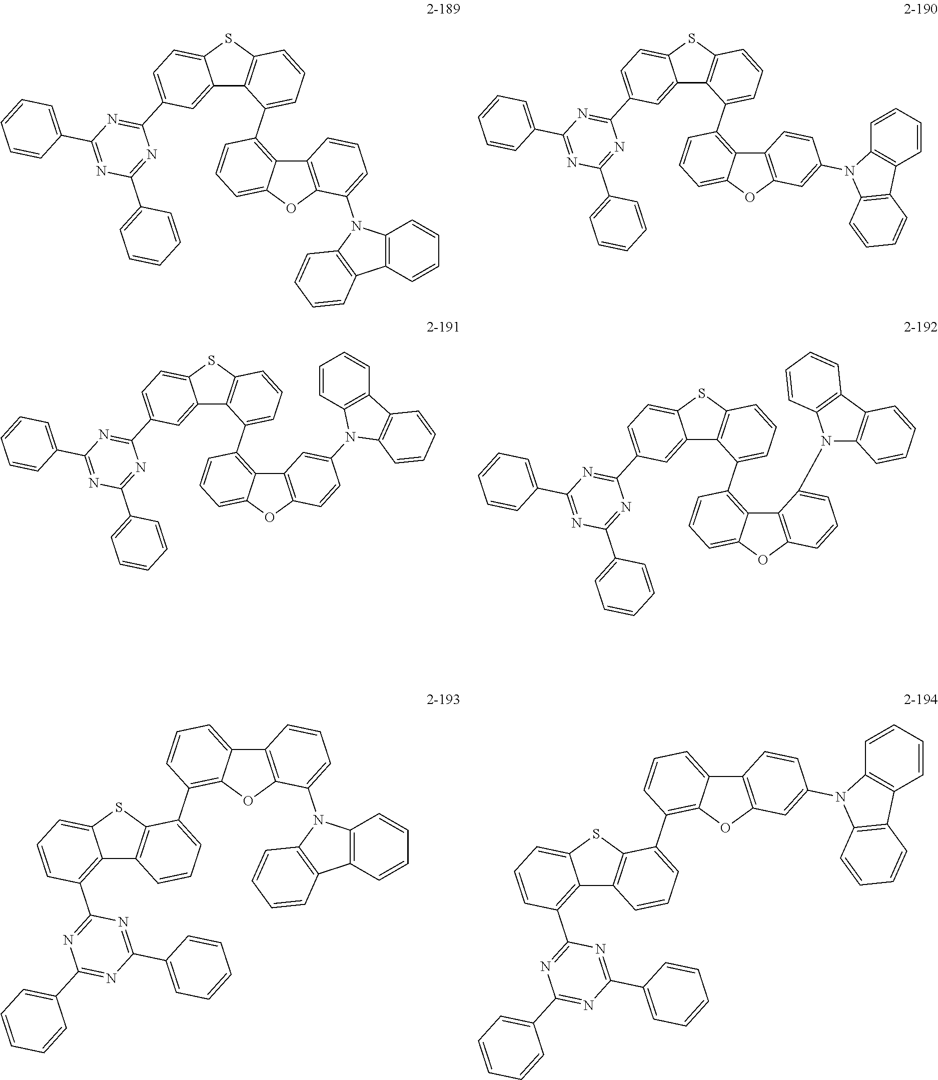

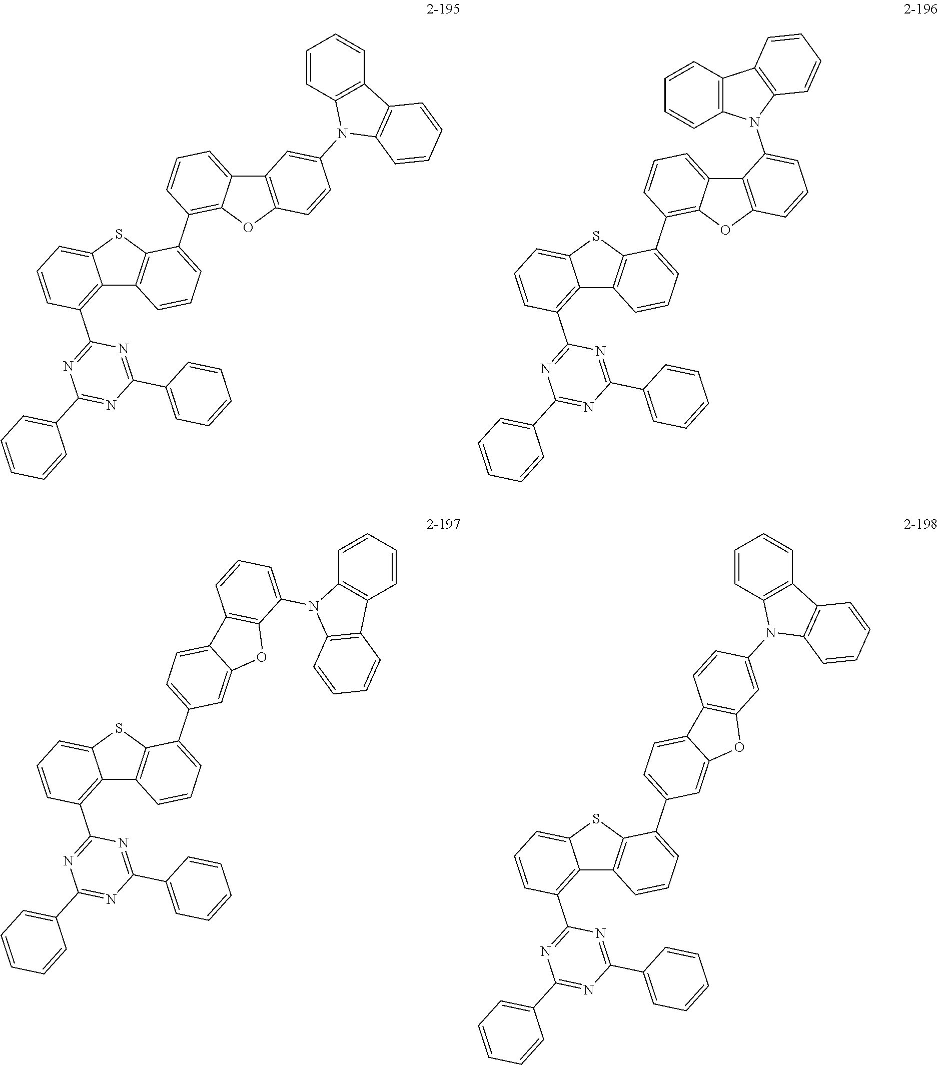 [SMILES: c1ccc(-c2nc(-c3ccccc3)nc(-c3cccc4sc5c(-c6cccc7c6oc6ccc(-n8c9ccccc9c9ccccc98)cc67)cccc5c34)n2)cc1 ; c1ccc(-c2nc(-c3ccccc3)nc(-c3cccc4sc5c(-c6cccc7c6oc6cccc(-n8c9ccccc9c9ccccc98)c67)cccc5c34)n2)cc1 ; c1ccc(-c2nc(-c3ccccc3)nc(-c3cccc4sc5c(-c6ccc7c(c6)oc6c(-n8c9ccccc9c9ccccc98)cccc67)cccc5c34)n2)cc1 ; c1ccc(-c2nc(-c3ccccc3)nc(-c3cccc4sc5c(-c6ccc7c(c6)oc6cc(-n8c9ccccc9c9ccccc98)ccc67)cccc5c34)n2)cc1]

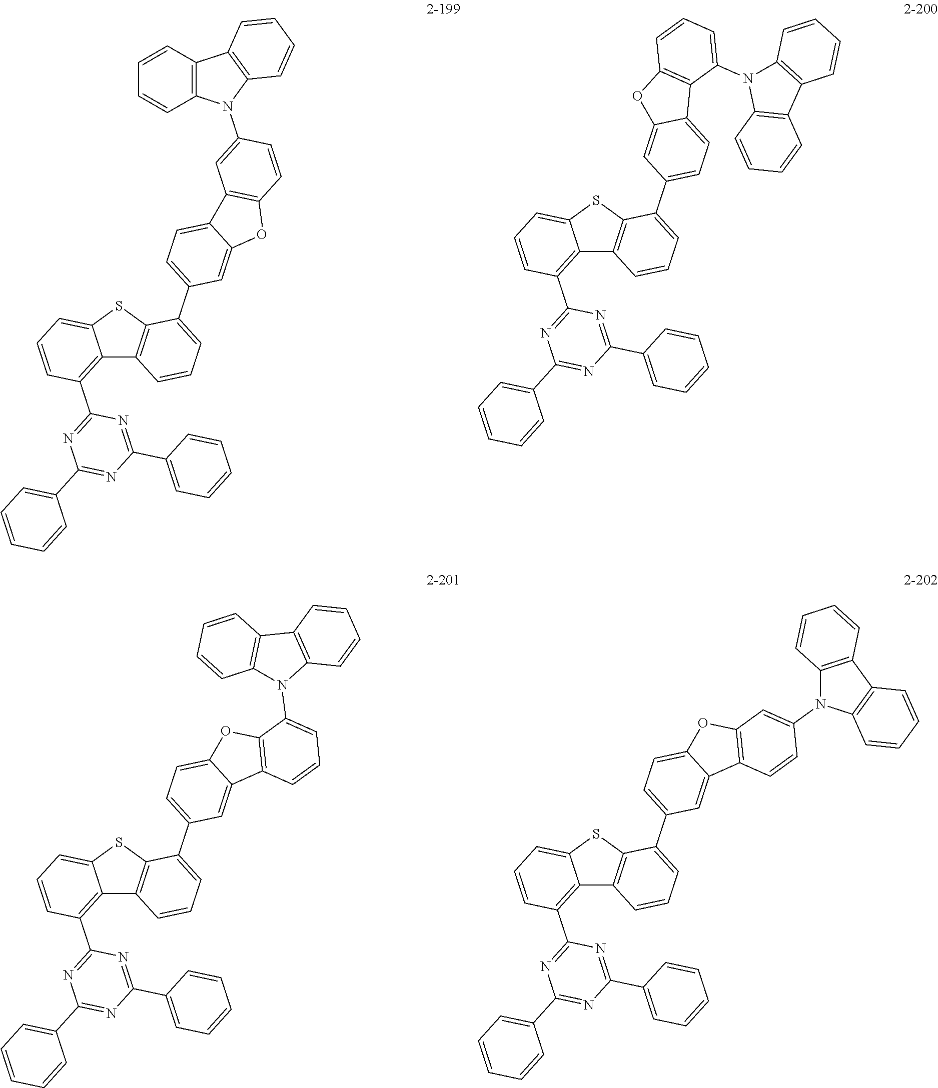 [SMILES: c1ccc(-c2nc(-c3ccccc3)nc(-c3cccc4sc5c(-c6ccc7c(c6)oc6ccc(-n8c9ccccc9c9ccccc98)cc67)cccc5c34)n2)cc1 ; c1ccc(-c2nc(-c3ccccc3)nc(-c3cccc4sc5c(-c6ccc7c(c6)oc6cccc(-n8c9ccccc9c9ccccc98)c67)cccc5c34)n2)cc1 ; c1ccc(-c2nc(-c3ccccc3)nc(-c3cccc4sc5c(-c6ccc7oc8c(-n9c%10ccccc%10c%10ccccc%109)cccc8c7c6)cccc5c34)n2)cc1 ; c1ccc(-c2nc(-c3ccccc3)nc(-c3cccc4sc5c(-c6ccc7oc8cc(-n9c%10ccccc%10c%10ccccc%109)ccc8c7c6)cccc5c34)n2)cc1]

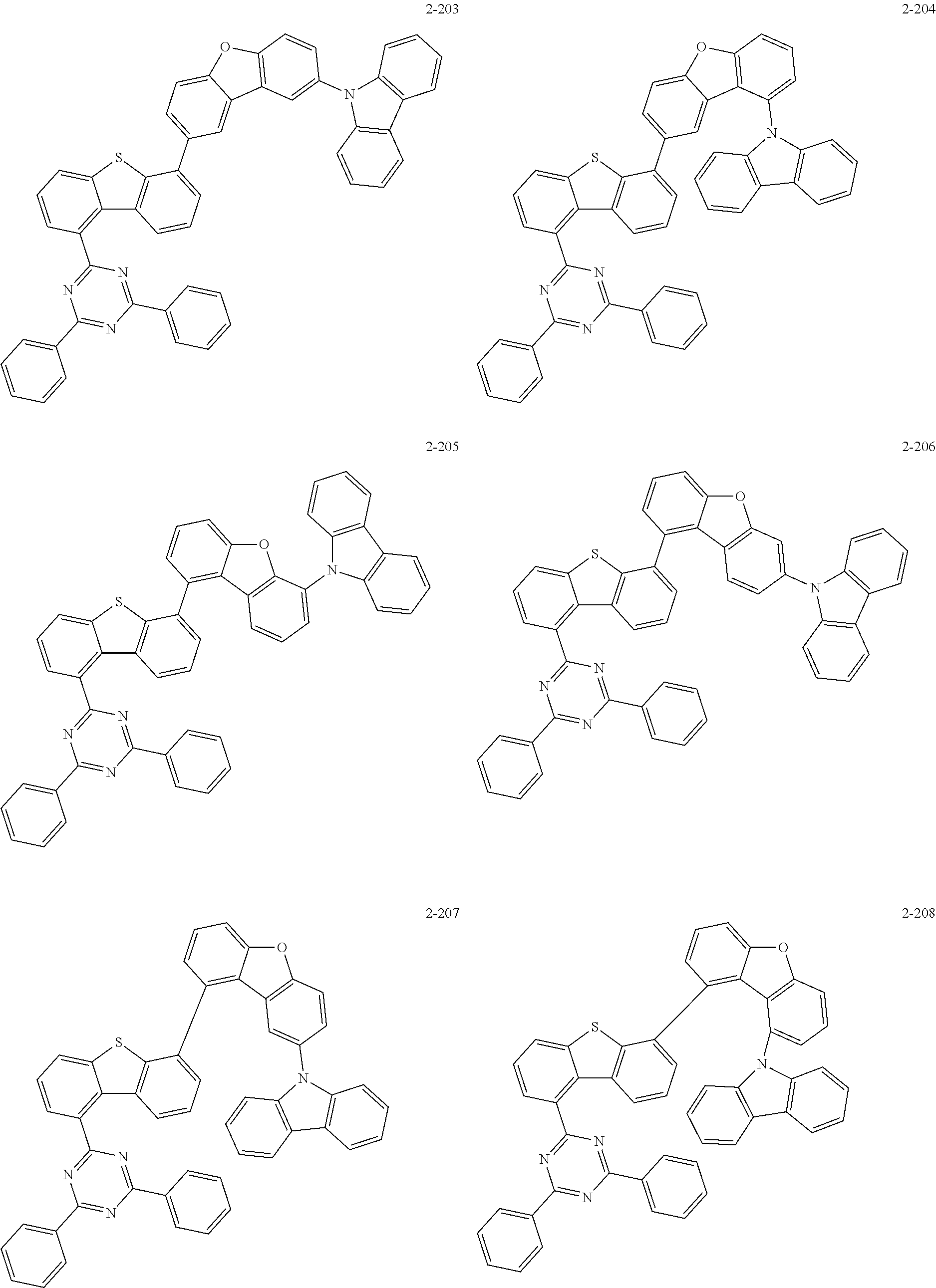

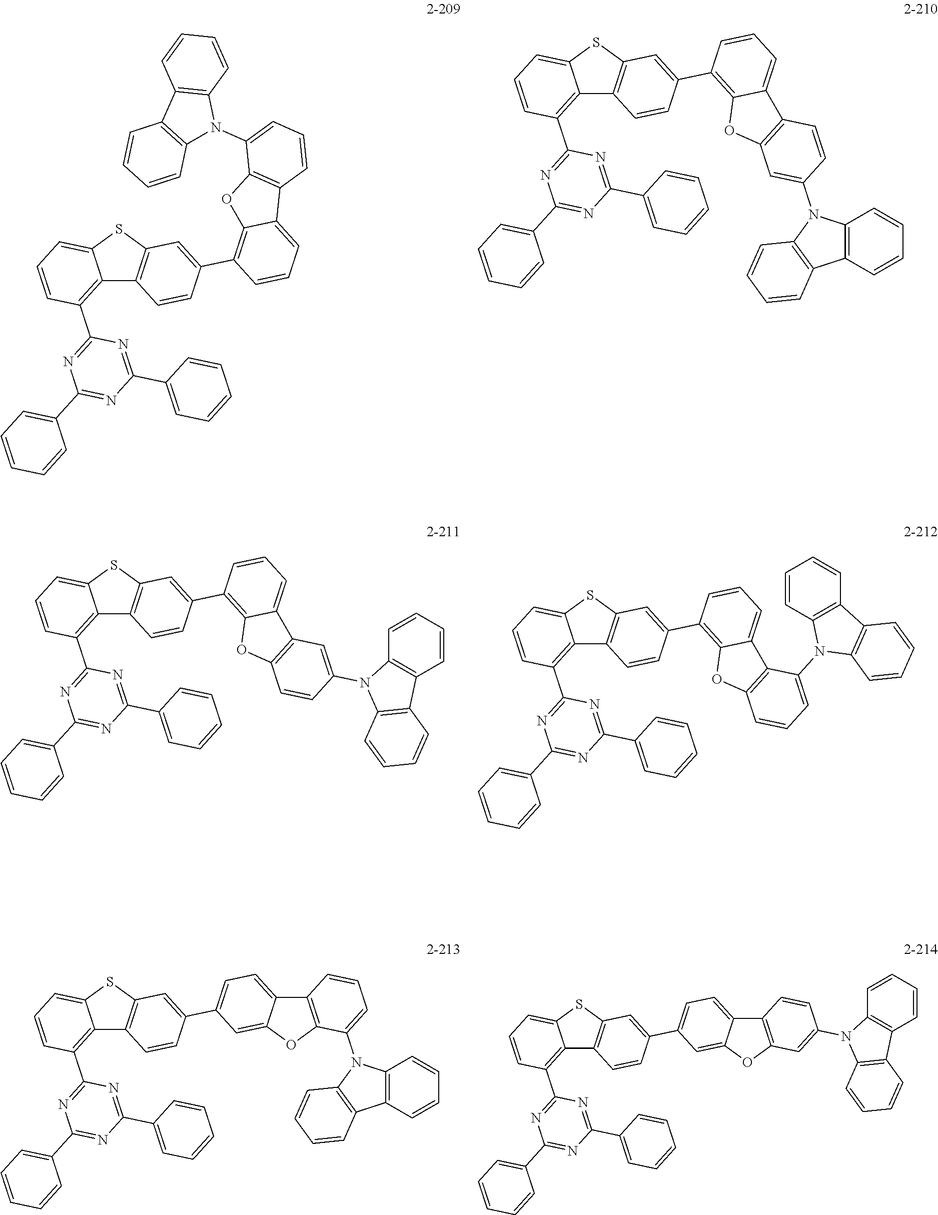

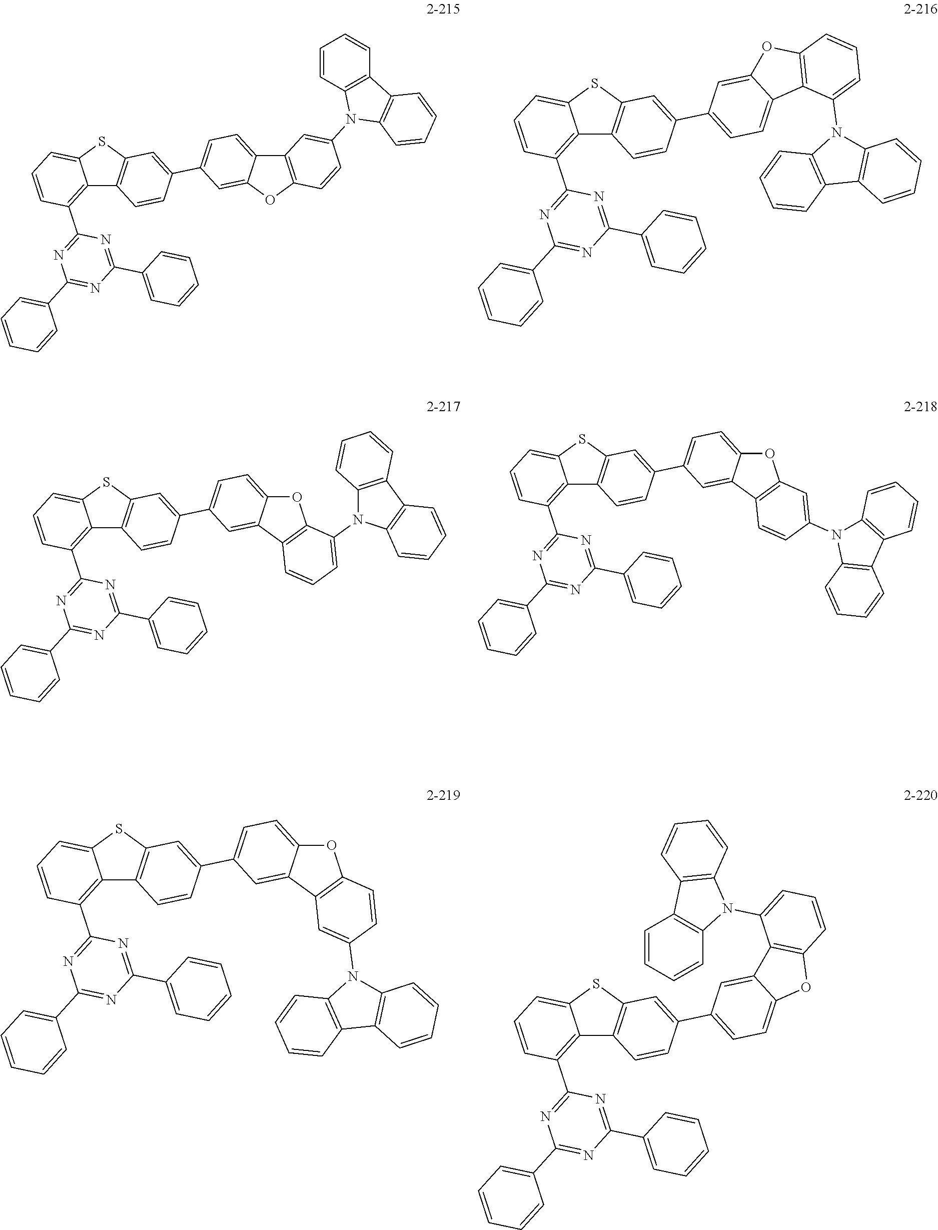

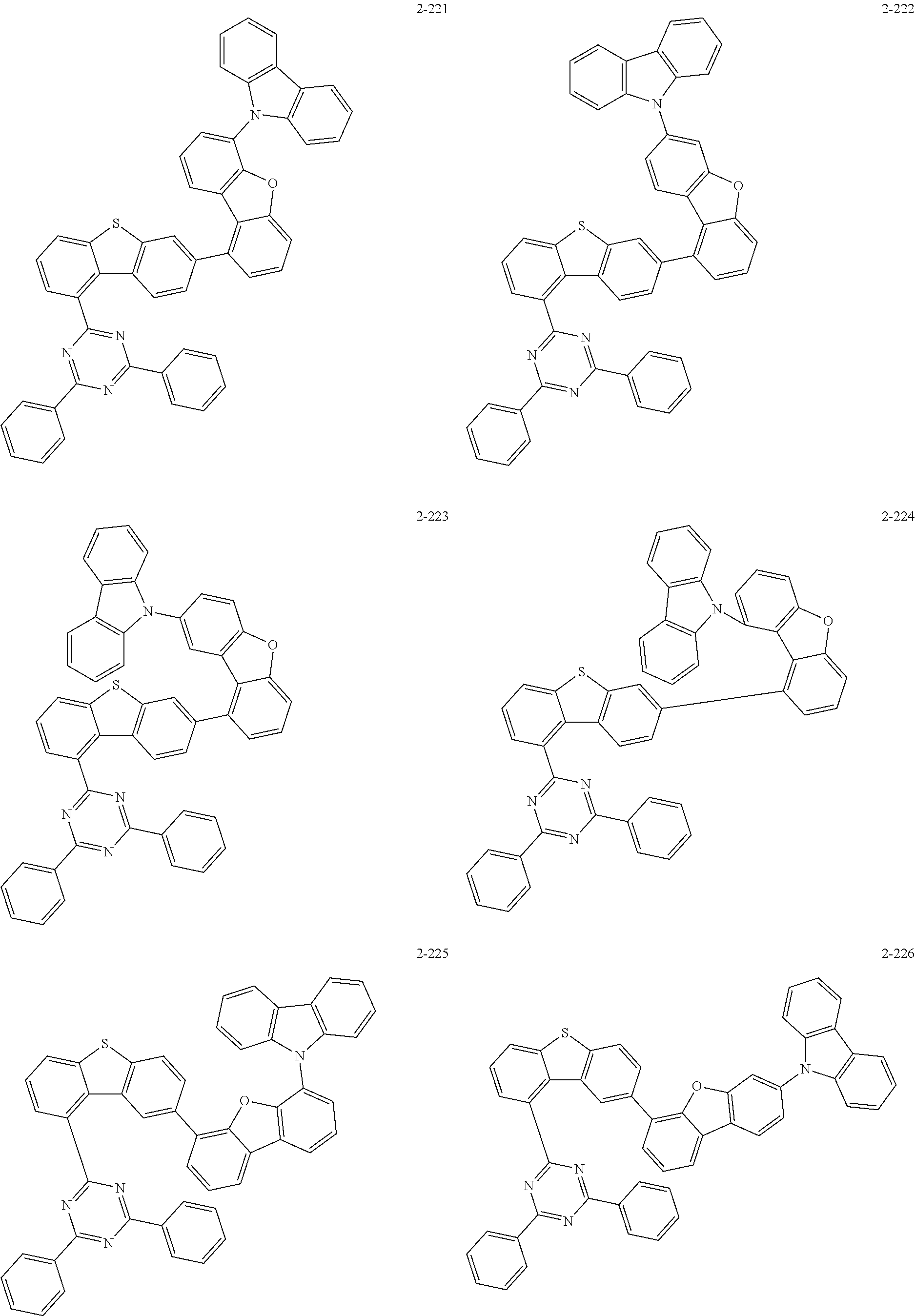

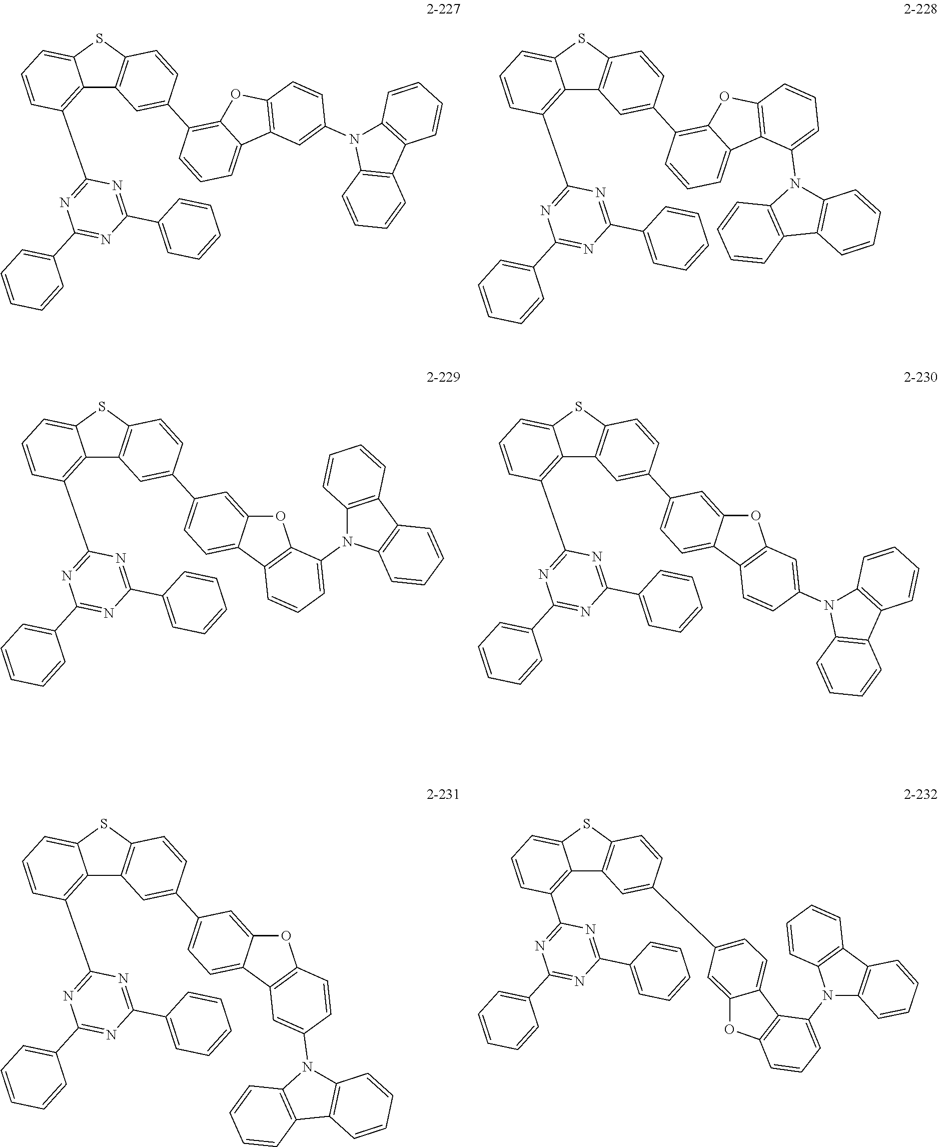

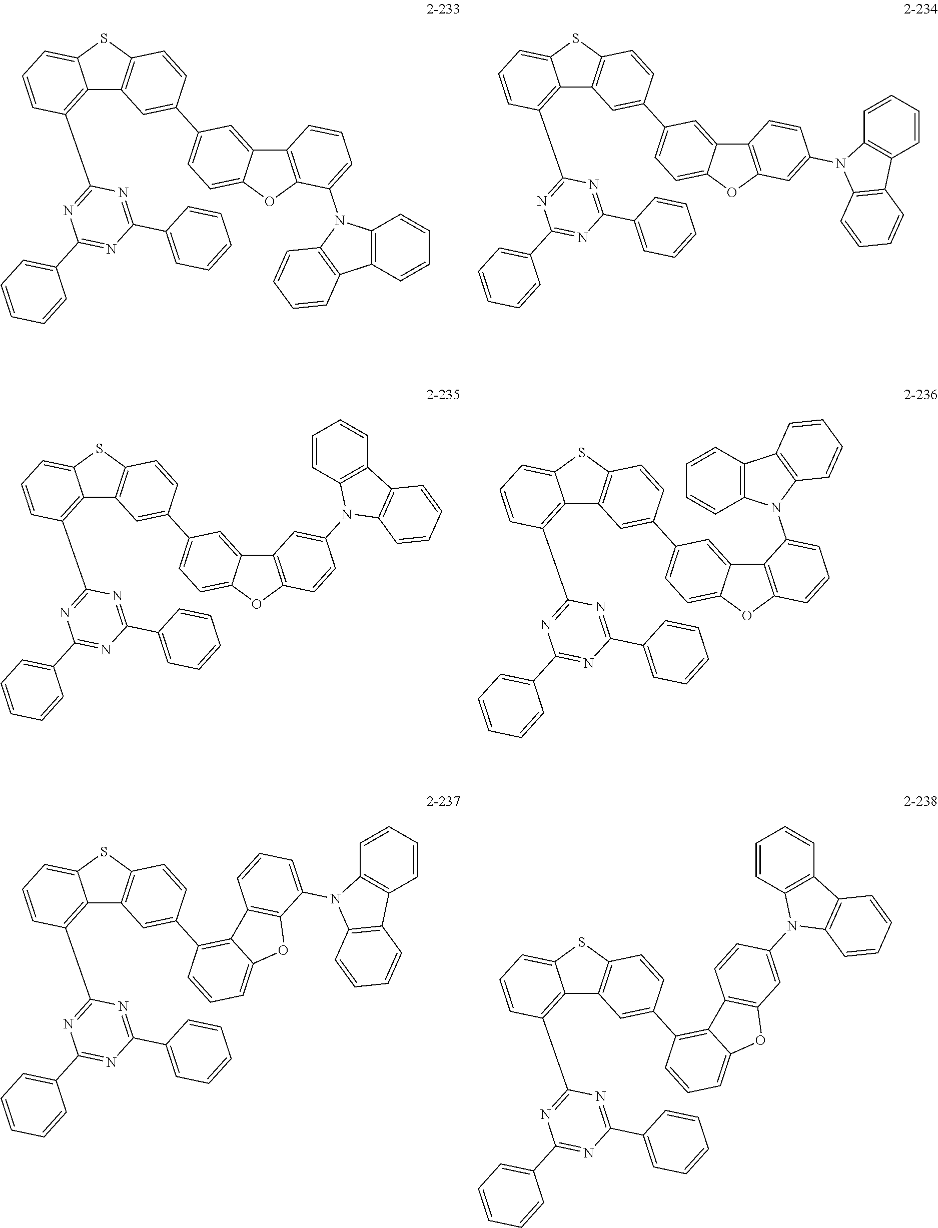

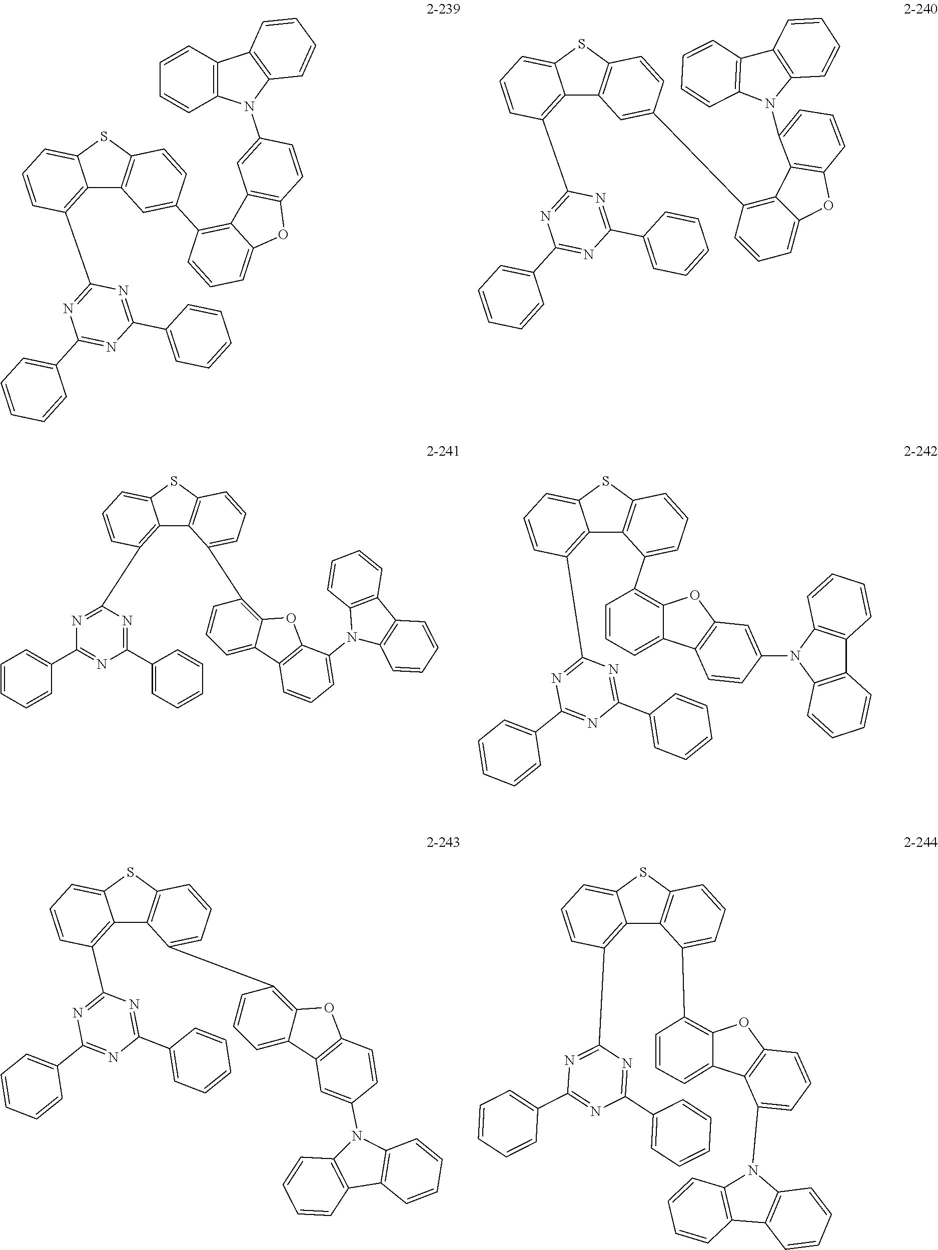

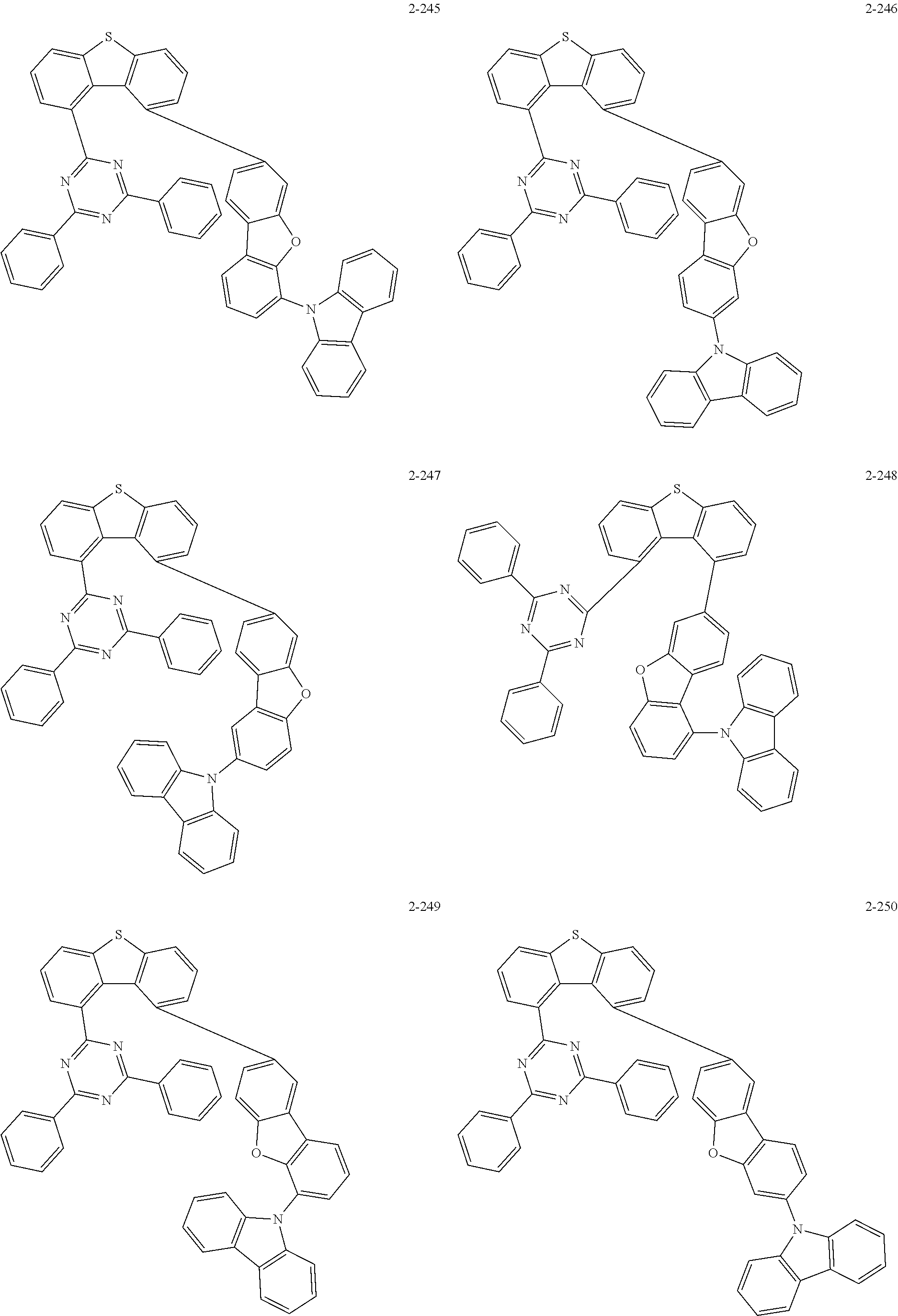

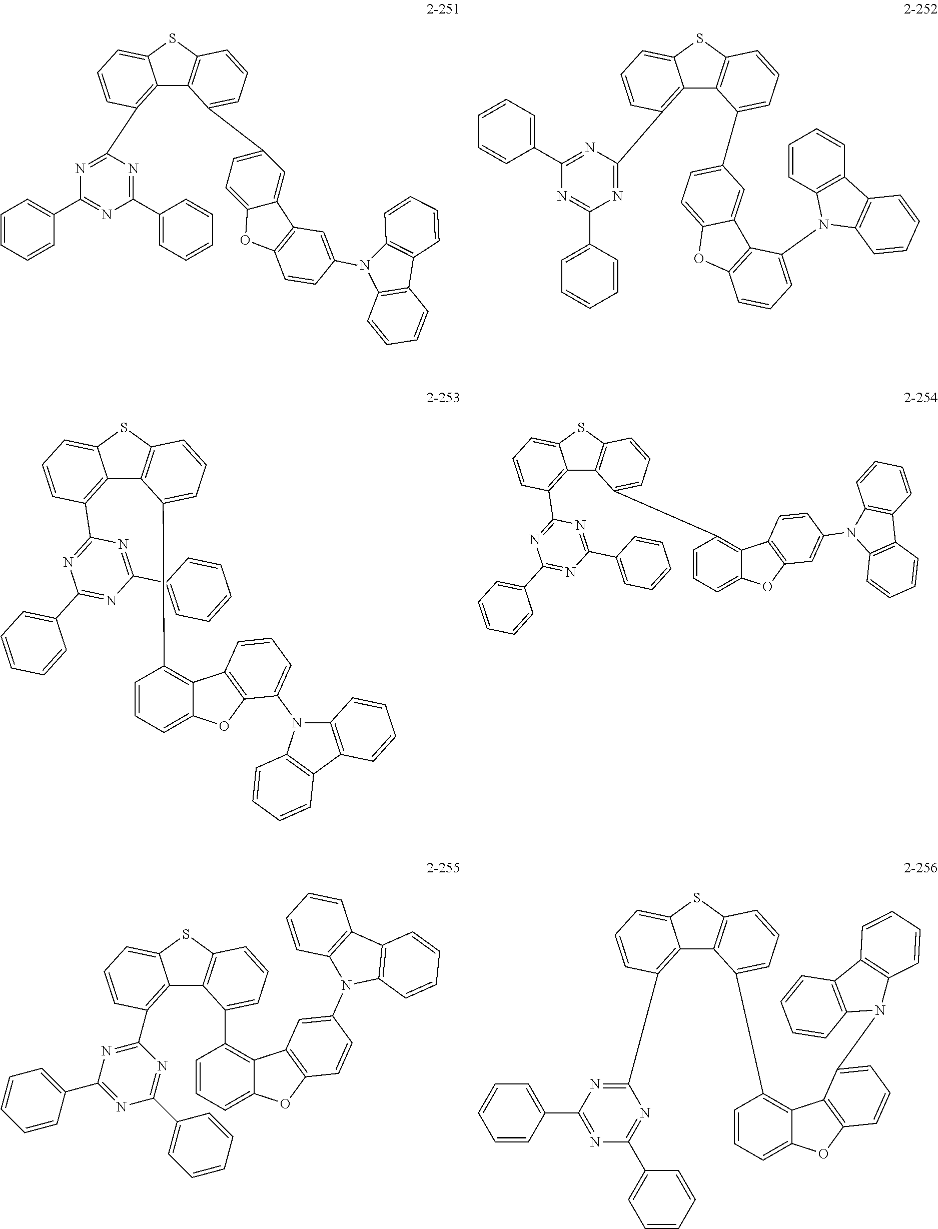

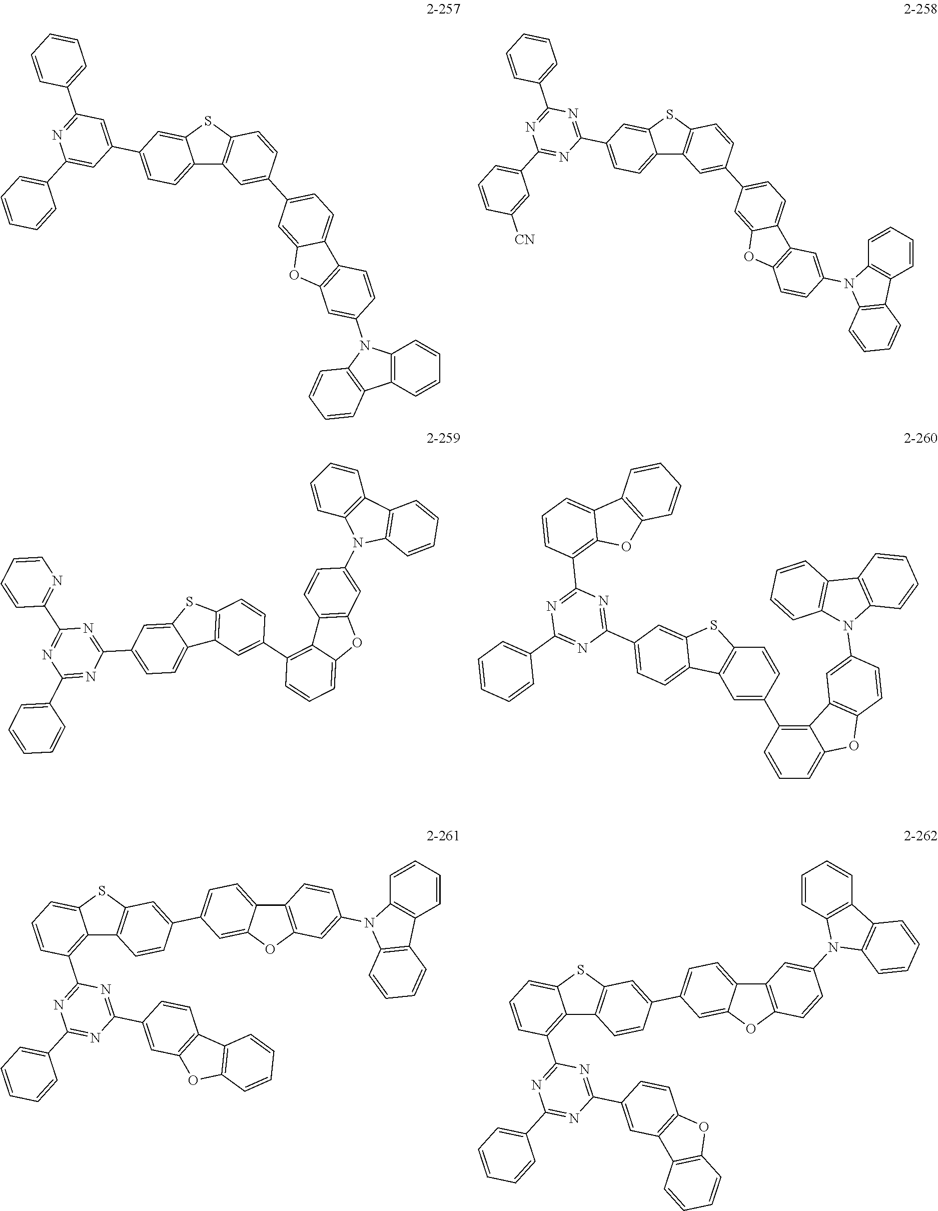

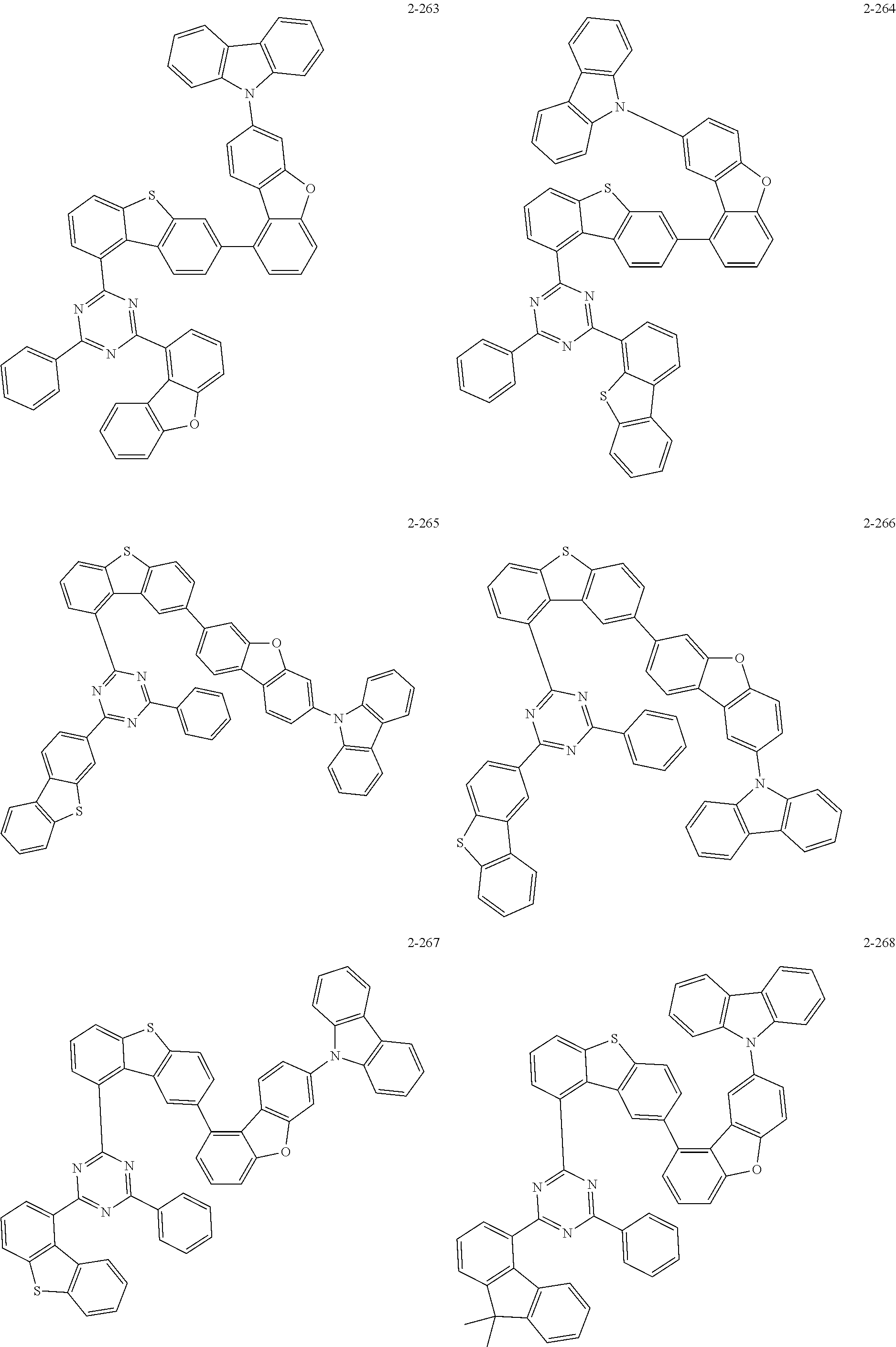

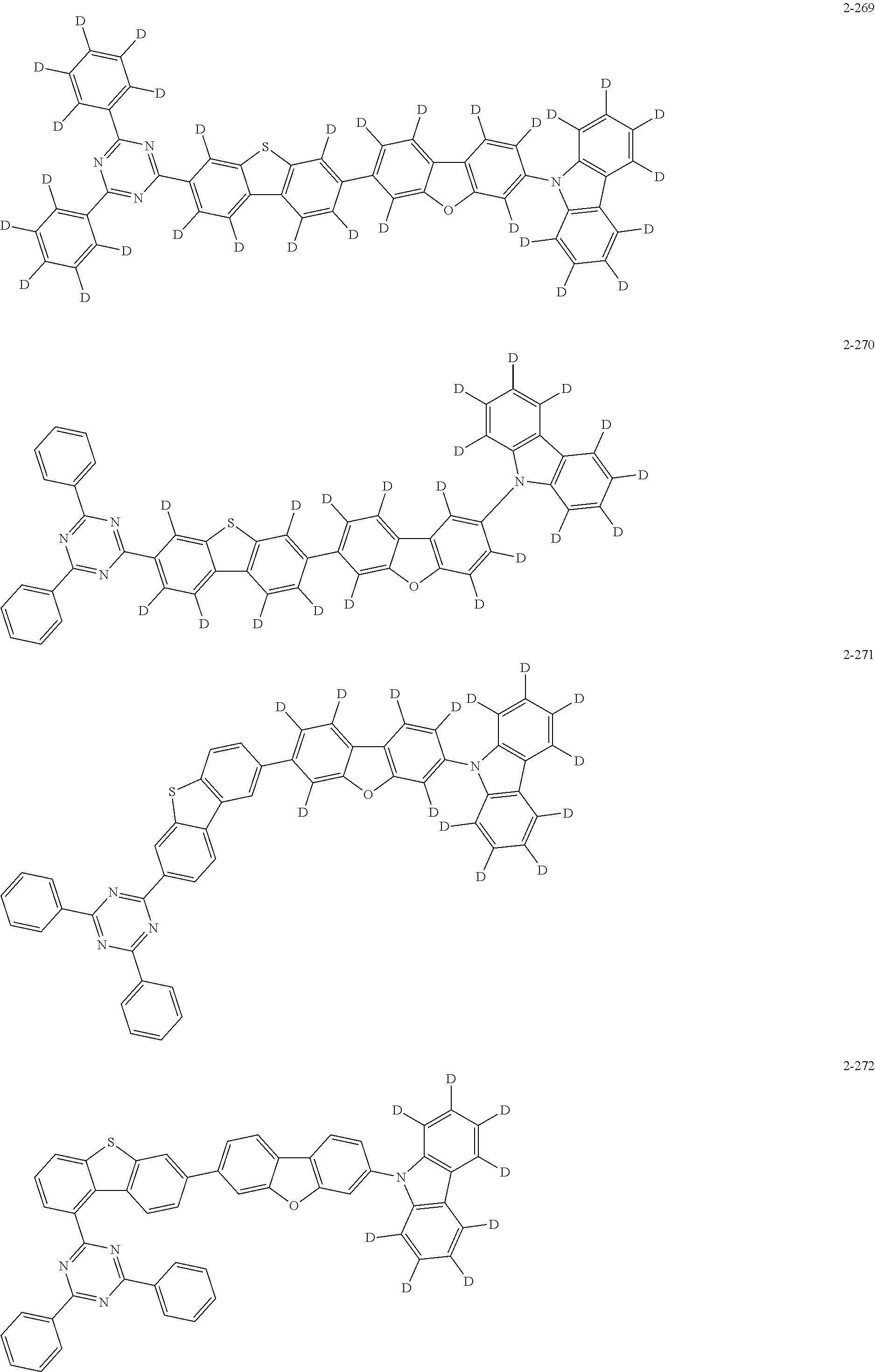

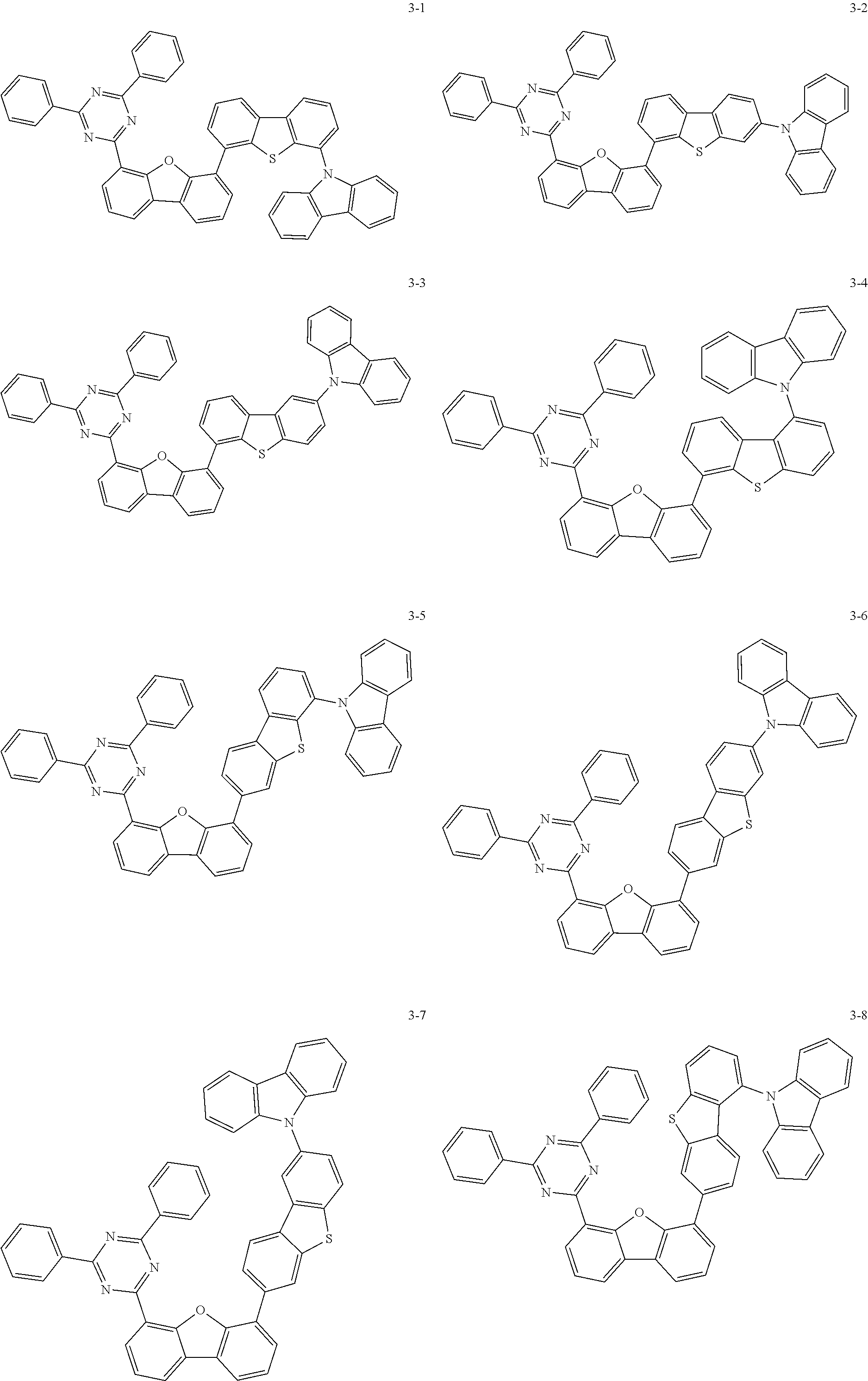

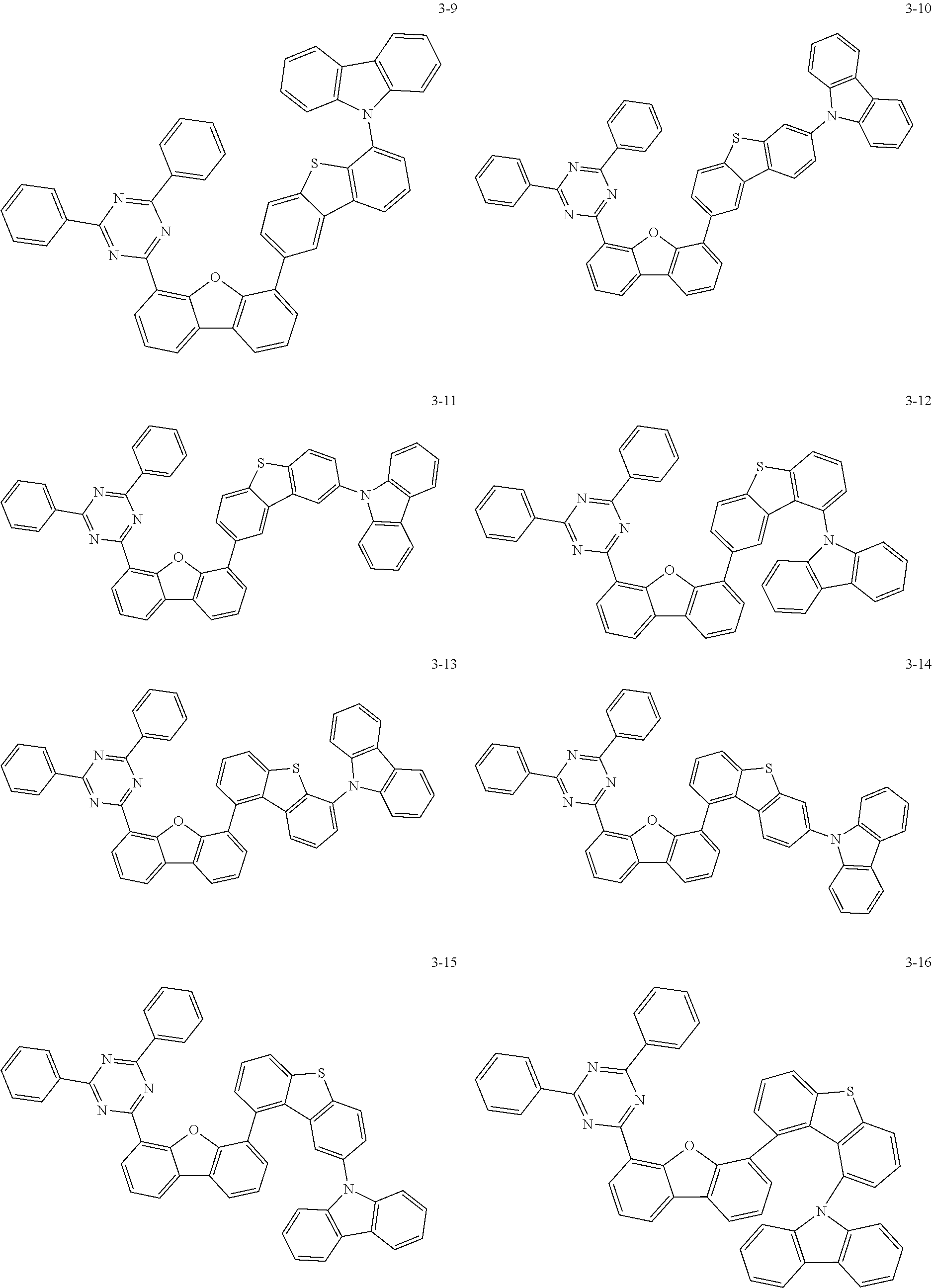

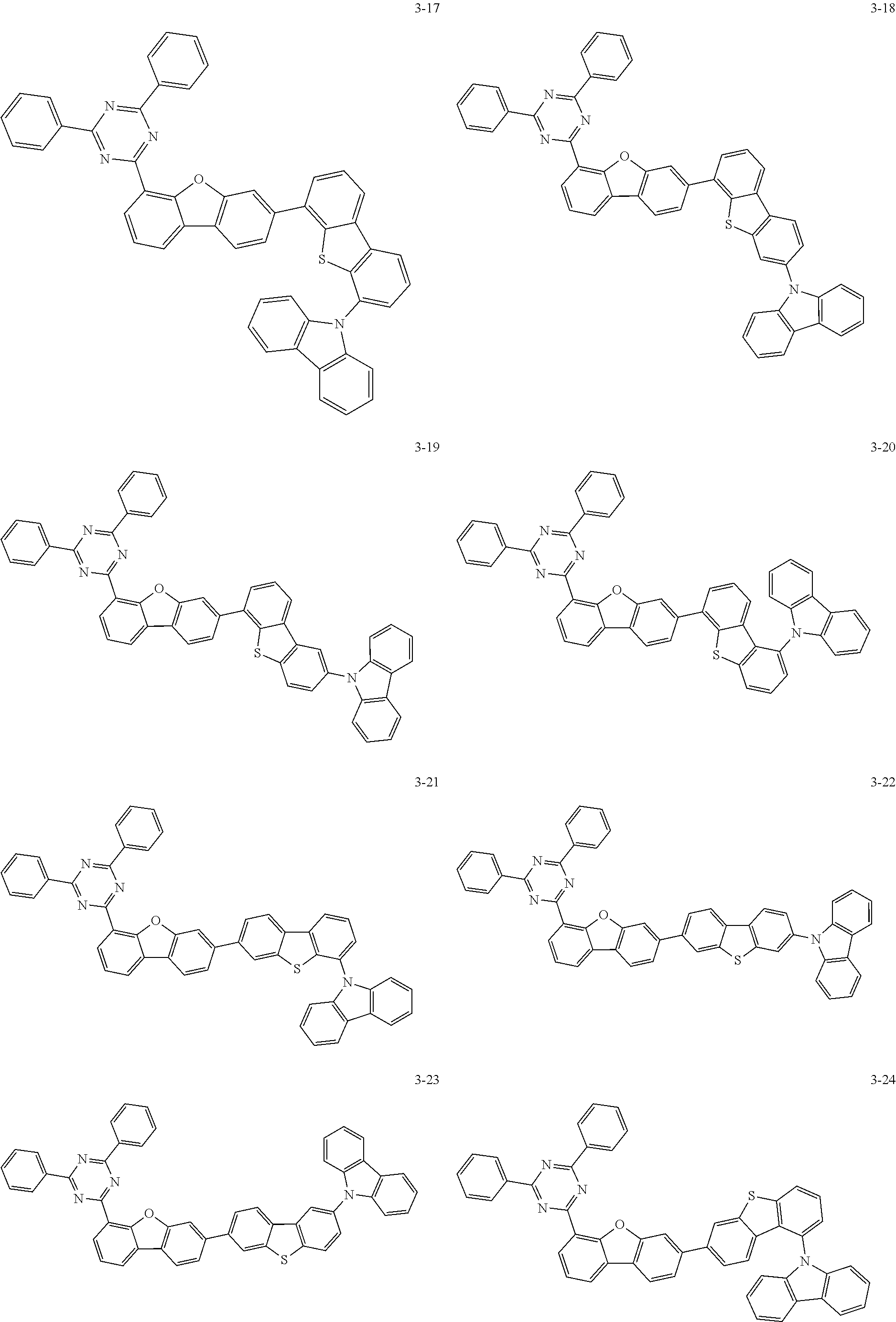

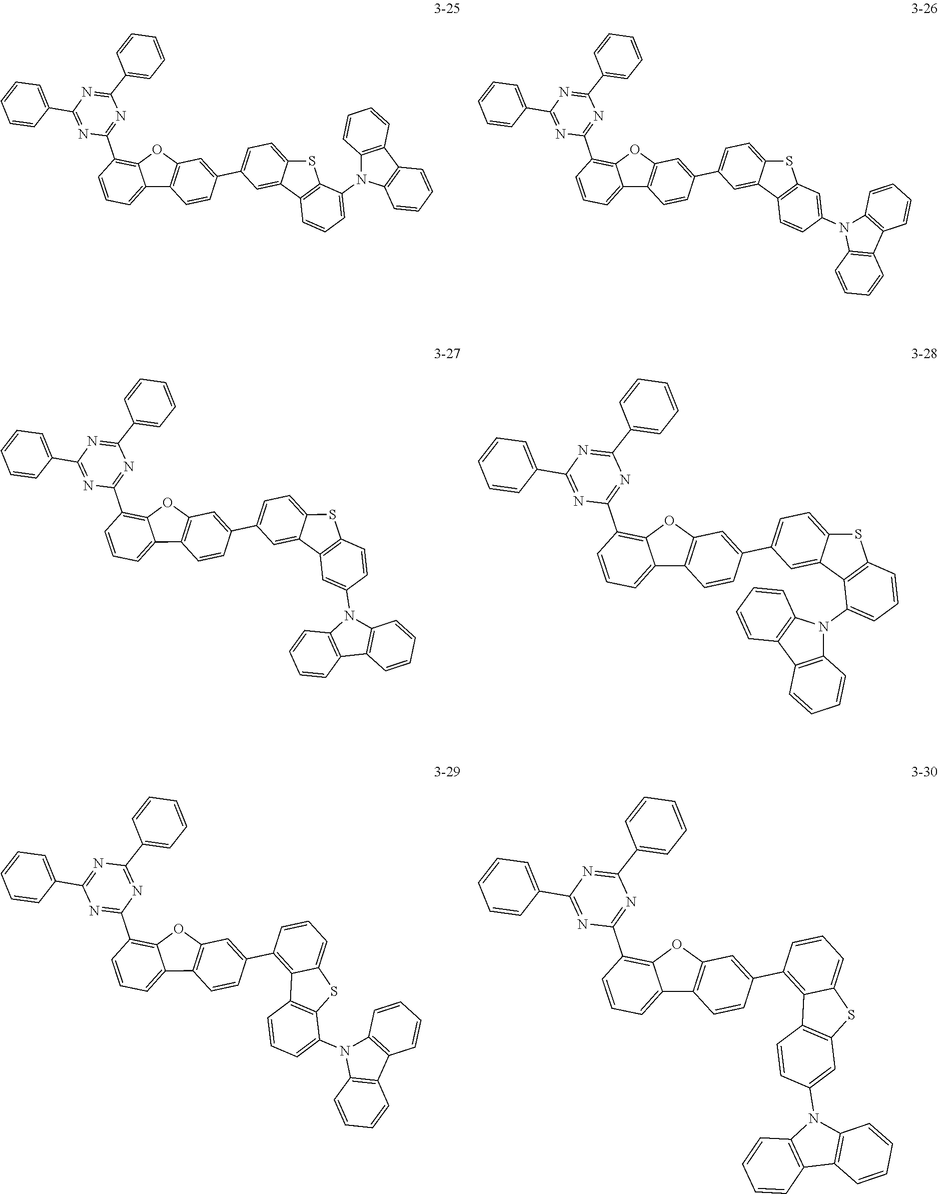

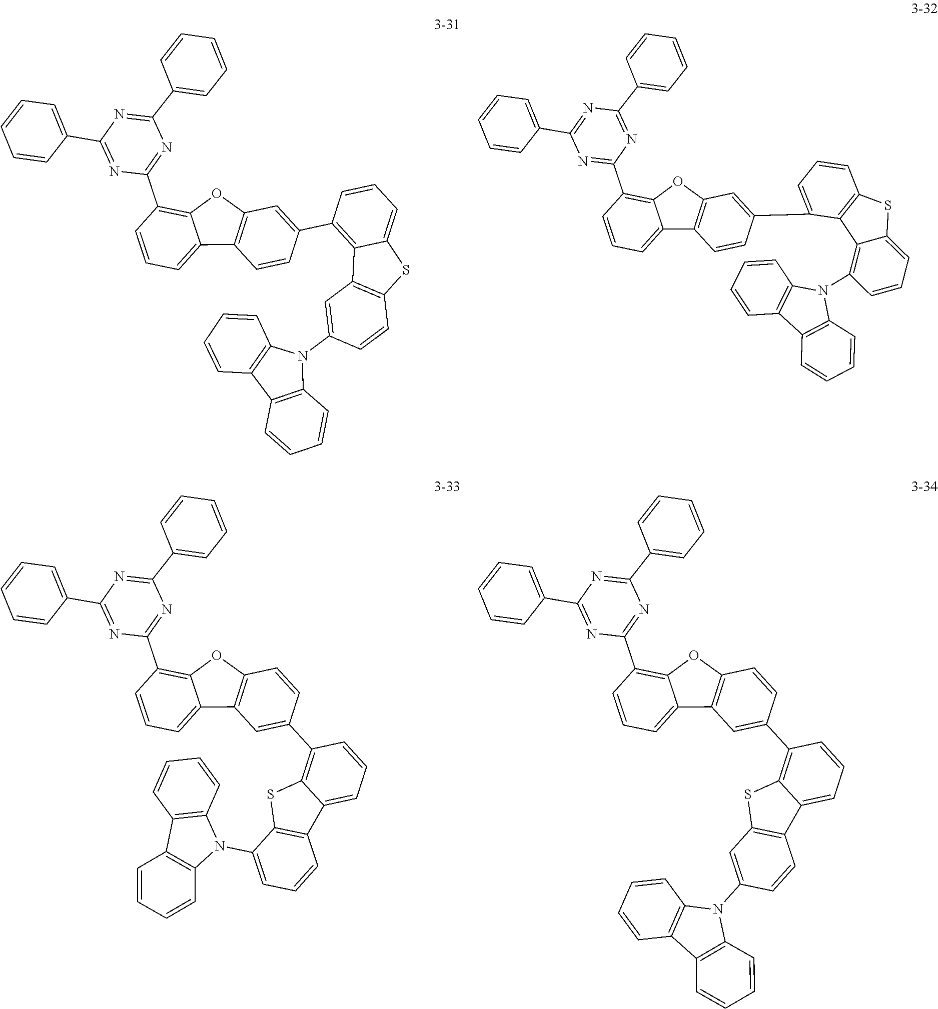 [SMILES: c1ccc(-c2nc(-c3ccccc3)nc(-c3cccc4c3oc3cc(-c5cccc6sc7ccc(-n8c9ccccc9c9ccccc98)cc7c56)ccc34)n2)cc1 ; c1ccc(-c2nc(-c3ccccc3)nc(-c3cccc4c3oc3cc(-c5cccc6sc7cccc(-n8c9ccccc9c9ccccc98)c7c56)ccc34)n2)cc1 ; c1ccc(-c2nc(-c3ccccc3)nc(-c3cccc4c3oc3ccc(-c5cccc6c5sc5c(-n7c8ccccc8c8ccccc87)cccc56)cc34)n2)cc1 ; c1ccc(-c2nc(-c3ccccc3)nc(-c3cccc4c3oc3ccc(-c5cccc6c5sc5cc(-n7c8ccccc8c8ccccc87)ccc56)cc34)n2)cc1]

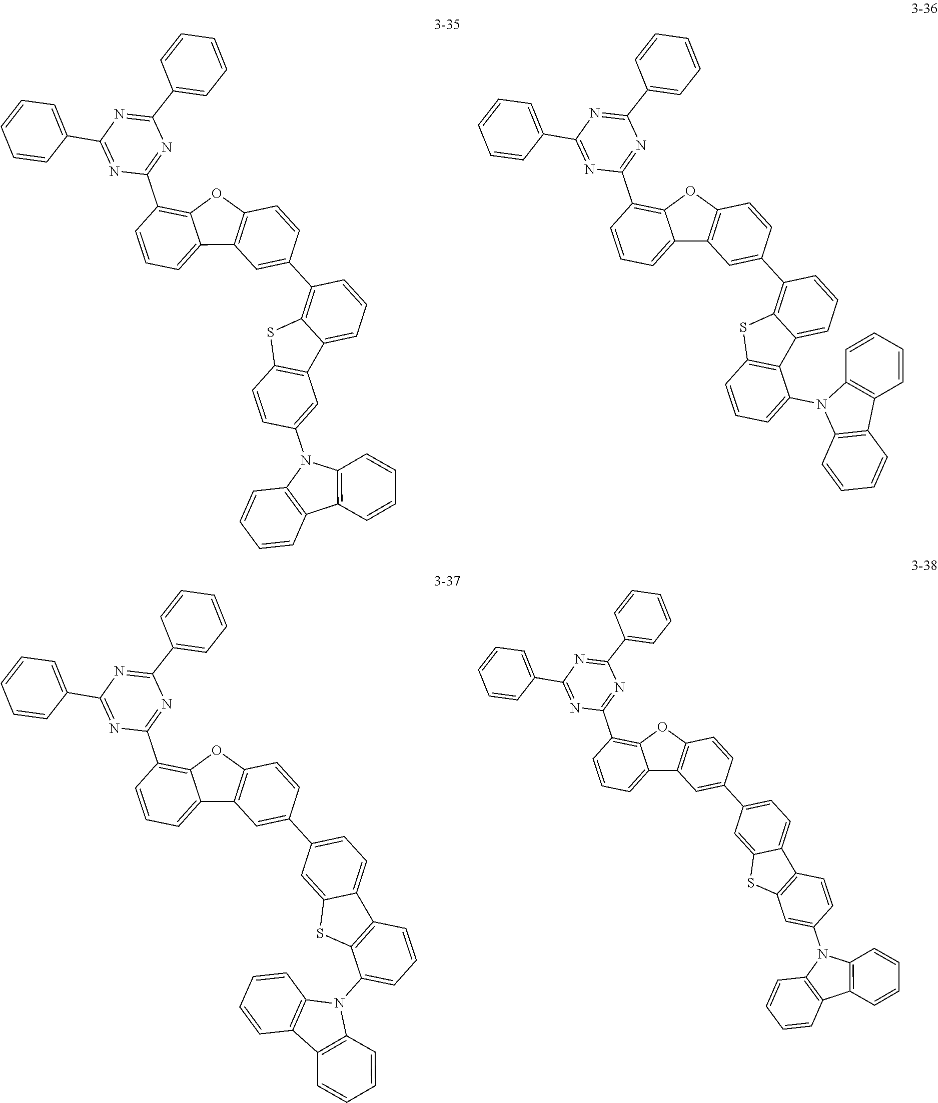 [SMILES: c1ccc(-c2nc(-c3ccccc3)nc(-c3cccc4c3oc3ccc(-c5cccc6c5sc5ccc(-n7c8ccccc8c8ccccc87)cc56)cc34)n2)cc1 ; c1ccc(-c2nc(-c3ccccc3)nc(-c3cccc4c3oc3ccc(-c5cccc6c5sc5cccc(-n7c8ccccc8c8ccccc87)c56)cc34)n2)cc1 ; c1ccc(-c2nc(-c3ccccc3)nc(-c3cccc4c3oc3ccc(-c5ccc6c(c5)sc5c(-n7c8ccccc8c8ccccc87)cccc56)cc34)n2)cc1 ; c1ccc(-c2nc(-c3ccccc3)nc(-c3cccc4c3oc3ccc(-c5ccc6c(c5)sc5cc(-n7c8ccccc8c8ccccc87)ccc56)cc34)n2)cc1]

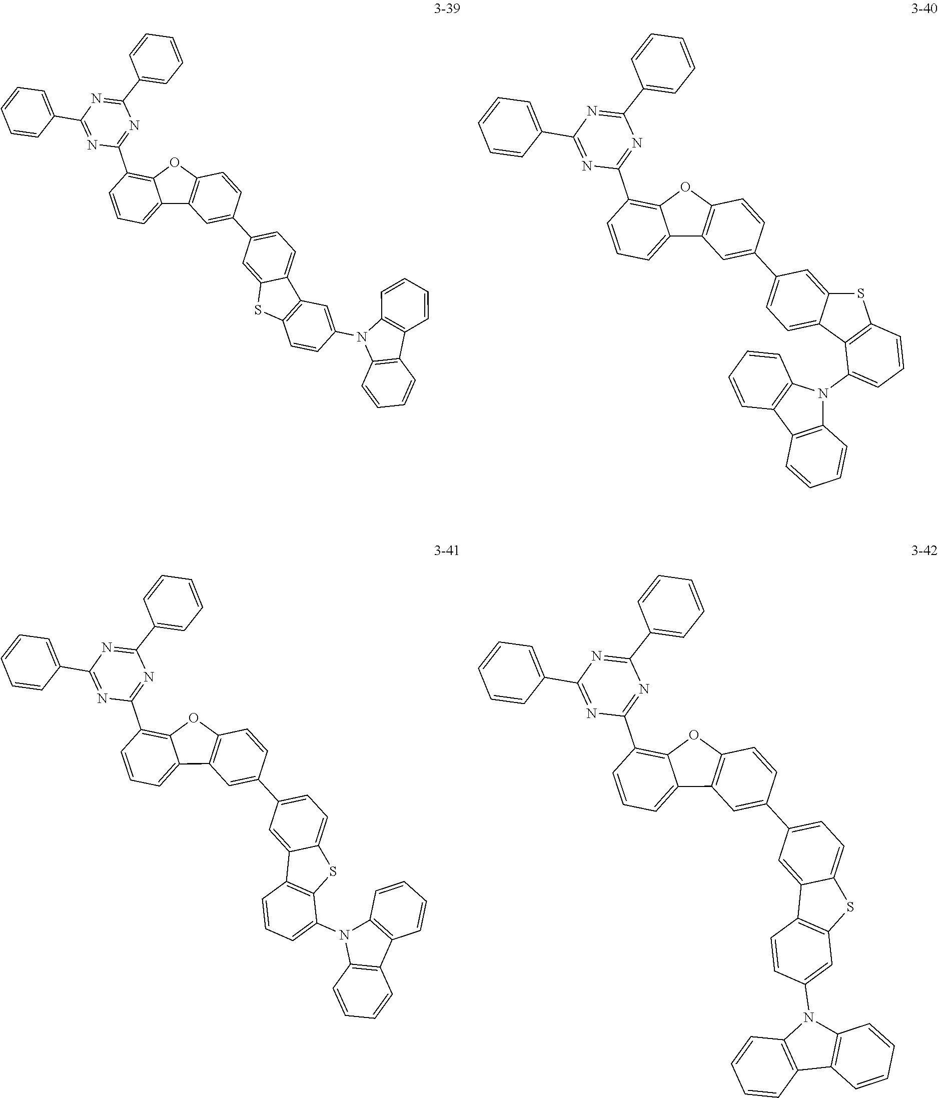 [SMILES: c1ccc(-c2nc(-c3ccccc3)nc(-c3cccc4c3oc3ccc(-c5ccc6c(c5)sc5ccc(-n7c8ccccc8c8ccccc87)cc56)cc34)n2)cc1 ; c1ccc(-c2nc(-c3ccccc3)nc(-c3cccc4c3oc3ccc(-c5ccc6c(c5)sc5cccc(-n7c8ccccc8c8ccccc87)c56)cc34)n2)cc1 ; c1ccc(-c2nc(-c3ccccc3)nc(-c3cccc4c3oc3ccc(-c5ccc6sc7c(-n8c9ccccc9c9ccccc98)cccc7c6c5)cc34)n2)cc1 ; c1ccc(-c2nc(-c3ccccc3)nc(-c3cccc4c3oc3ccc(-c5ccc6sc7cc(-n8c9ccccc9c9ccccc98)ccc7c6c5)cc34)n2)cc1]

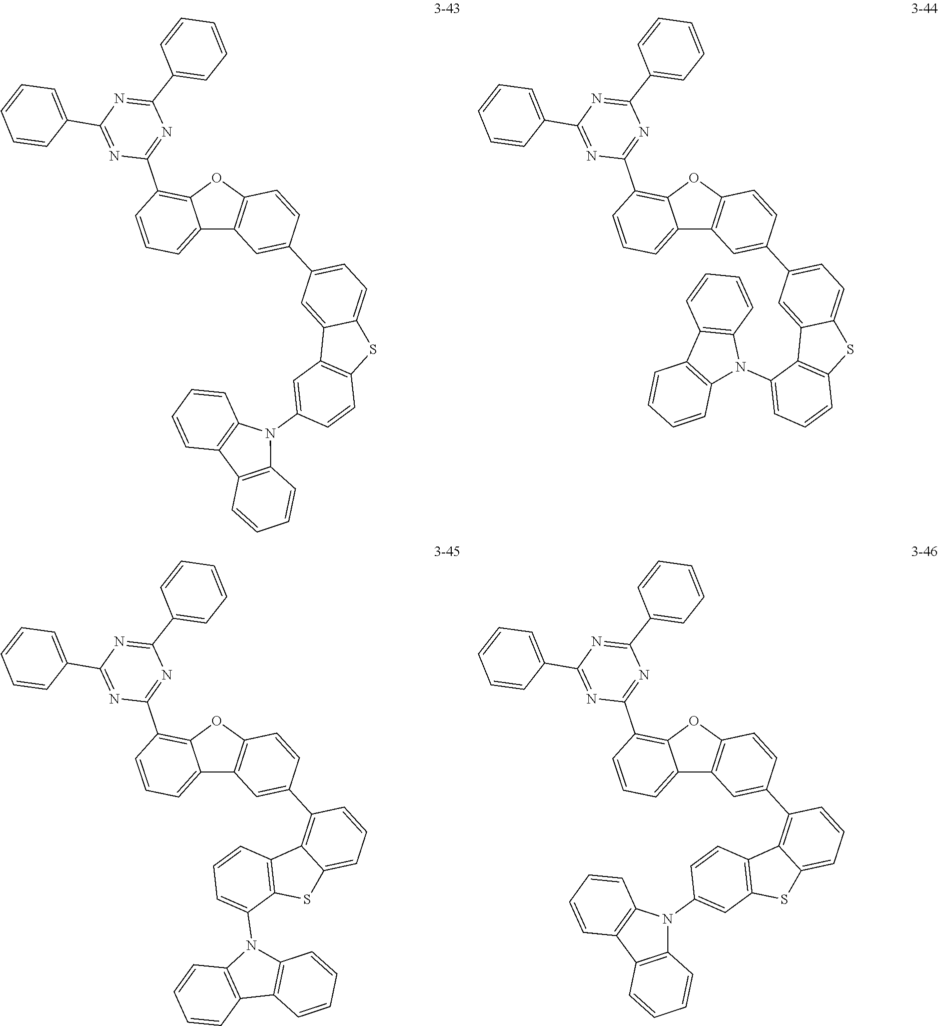 [SMILES: c1ccc(-c2nc(-c3ccccc3)nc(-c3cccc4c3oc3ccc(-c5ccc6sc7ccc(-n8c9ccccc9c9ccccc98)cc7c6c5)cc34)n2)cc1 ; c1ccc(-c2nc(-c3ccccc3)nc(-c3cccc4c3oc3ccc(-c5ccc6sc7cccc(-n8c9ccccc9c9ccccc98)c7c6c5)cc34)n2)cc1 ; c1ccc(-c2nc(-c3ccccc3)nc(-c3cccc4c3oc3ccc(-c5cccc6sc7c(-n8c9ccccc9c9ccccc98)cccc7c56)cc34)n2)cc1 ; c1ccc(-c2nc(-c3ccccc3)nc(-c3cccc4c3oc3ccc(-c5cccc6sc7cc(-n8c9ccccc9c9ccccc98)ccc7c56)cc34)n2)cc1]

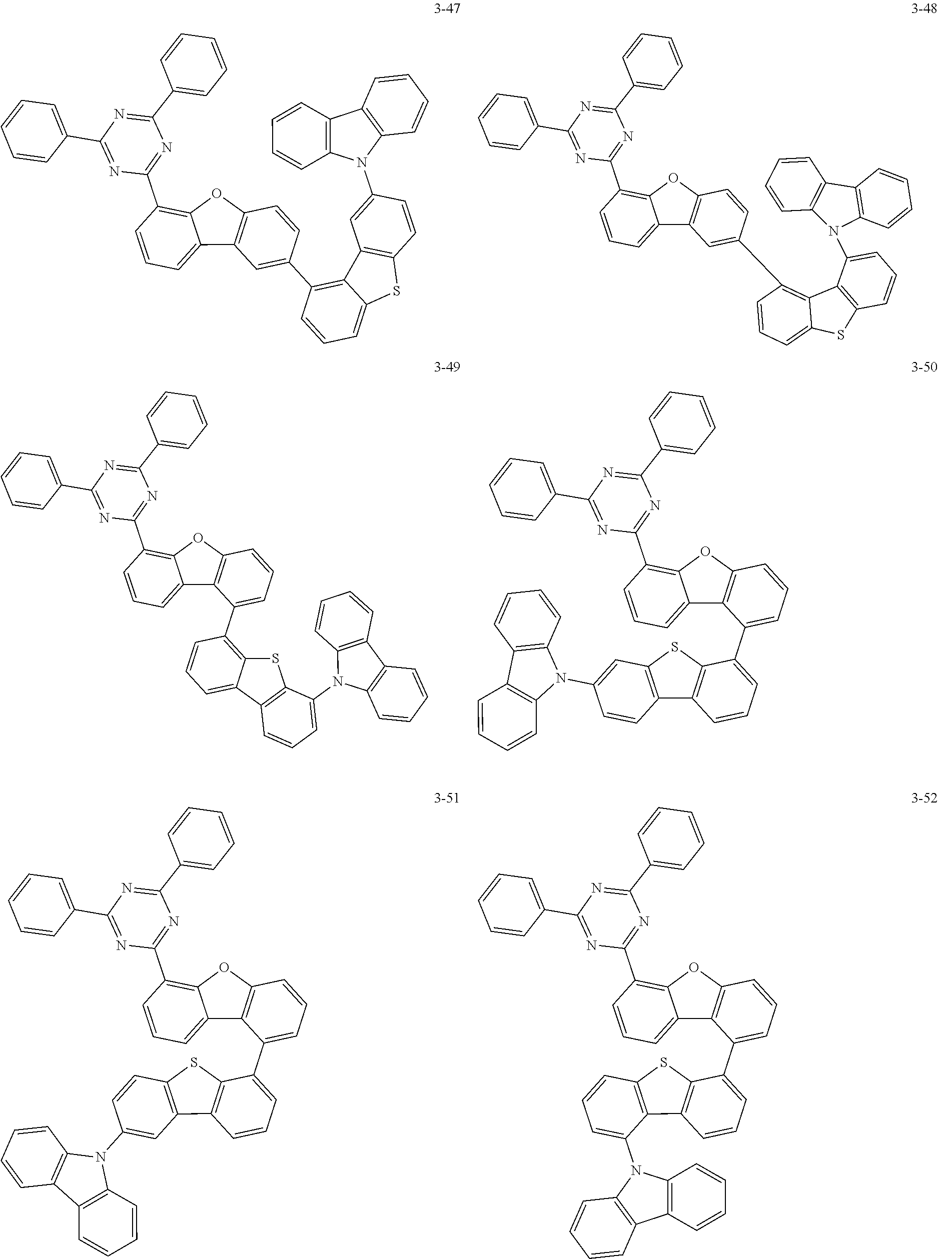

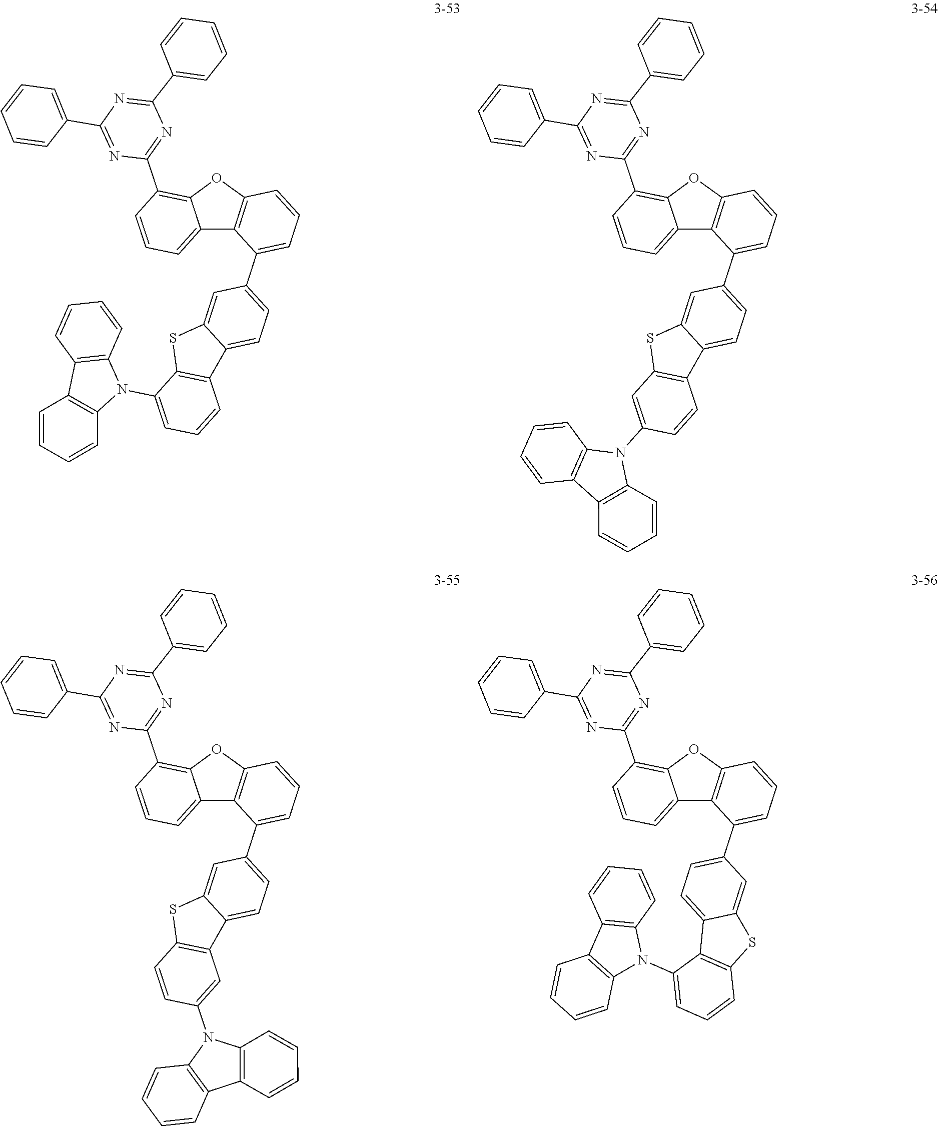 [SMILES: c1ccc(-c2nc(-c3ccccc3)nc(-c3cccc4c3oc3cccc(-c5ccc6c(c5)sc5c(-n7c8ccccc8c8ccccc87)cccc56)c34)n2)cc1 ; c1ccc(-c2nc(-c3ccccc3)nc(-c3cccc4c3oc3cccc(-c5ccc6c(c5)sc5cc(-n7c8ccccc8c8ccccc87)ccc56)c34)n2)cc1 ; c1ccc(-c2nc(-c3ccccc3)nc(-c3cccc4c3oc3cccc(-c5ccc6c(c5)sc5ccc(-n7c8ccccc8c8ccccc87)cc56)c34)n2)cc1 ; c1ccc(-c2nc(-c3ccccc3)nc(-c3cccc4c3oc3cccc(-c5ccc6c(c5)sc5cccc(-n7c8ccccc8c8ccccc87)c56)c34)n2)cc1]

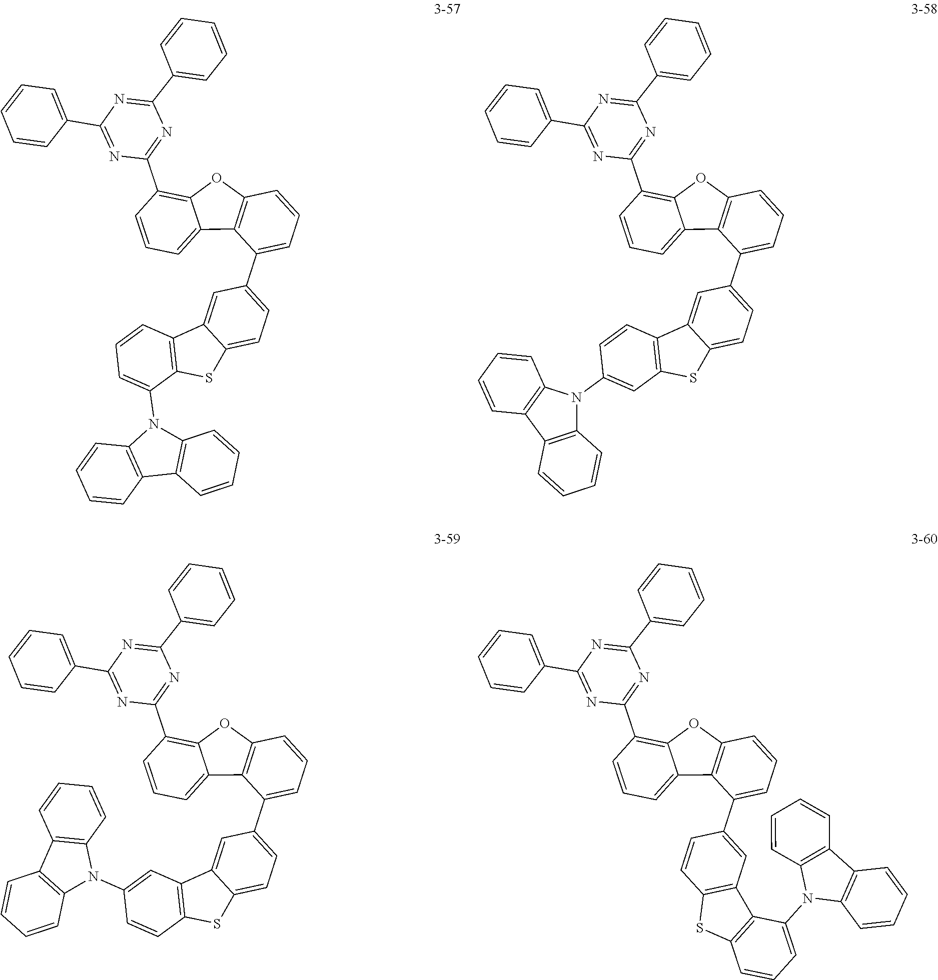 [SMILES: c1ccc(-c2nc(-c3ccccc3)nc(-c3cccc4c3oc3cccc(-c5ccc6sc7c(-n8c9ccccc9c9ccccc98)cccc7c6c5)c34)n2)cc1 ; c1ccc(-c2nc(-c3ccccc3)nc(-c3cccc4c3oc3cccc(-c5ccc6sc7cc(-n8c9ccccc9c9ccccc98)ccc7c6c5)c34)n2)cc1 ; c1ccc(-c2nc(-c3ccccc3)nc(-c3cccc4c3oc3cccc(-c5ccc6sc7ccc(-n8c9ccccc9c9ccccc98)cc7c6c5)c34)n2)cc1 ; c1ccc(-c2nc(-c3ccccc3)nc(-c3cccc4c3oc3cccc(-c5ccc6sc7cccc(-n8c9ccccc9c9ccccc98)c7c6c5)c34)n2)cc1]

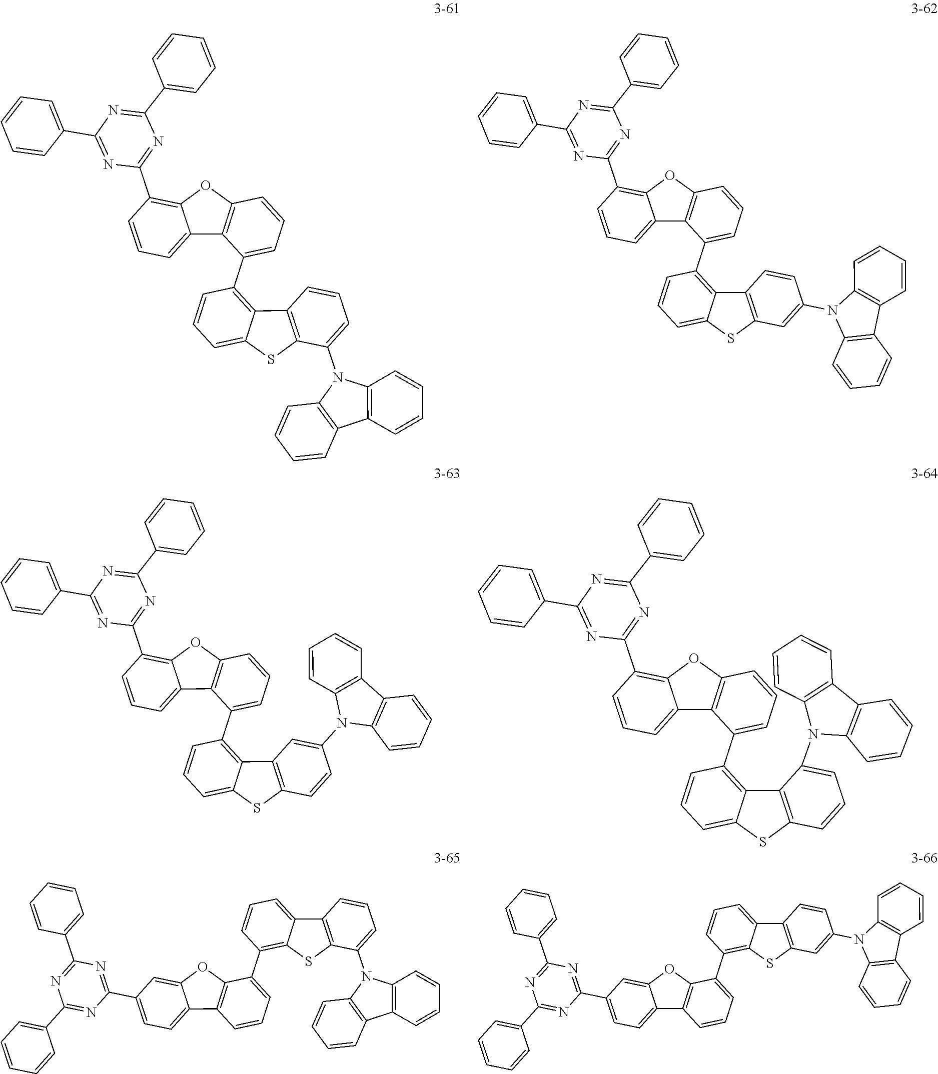

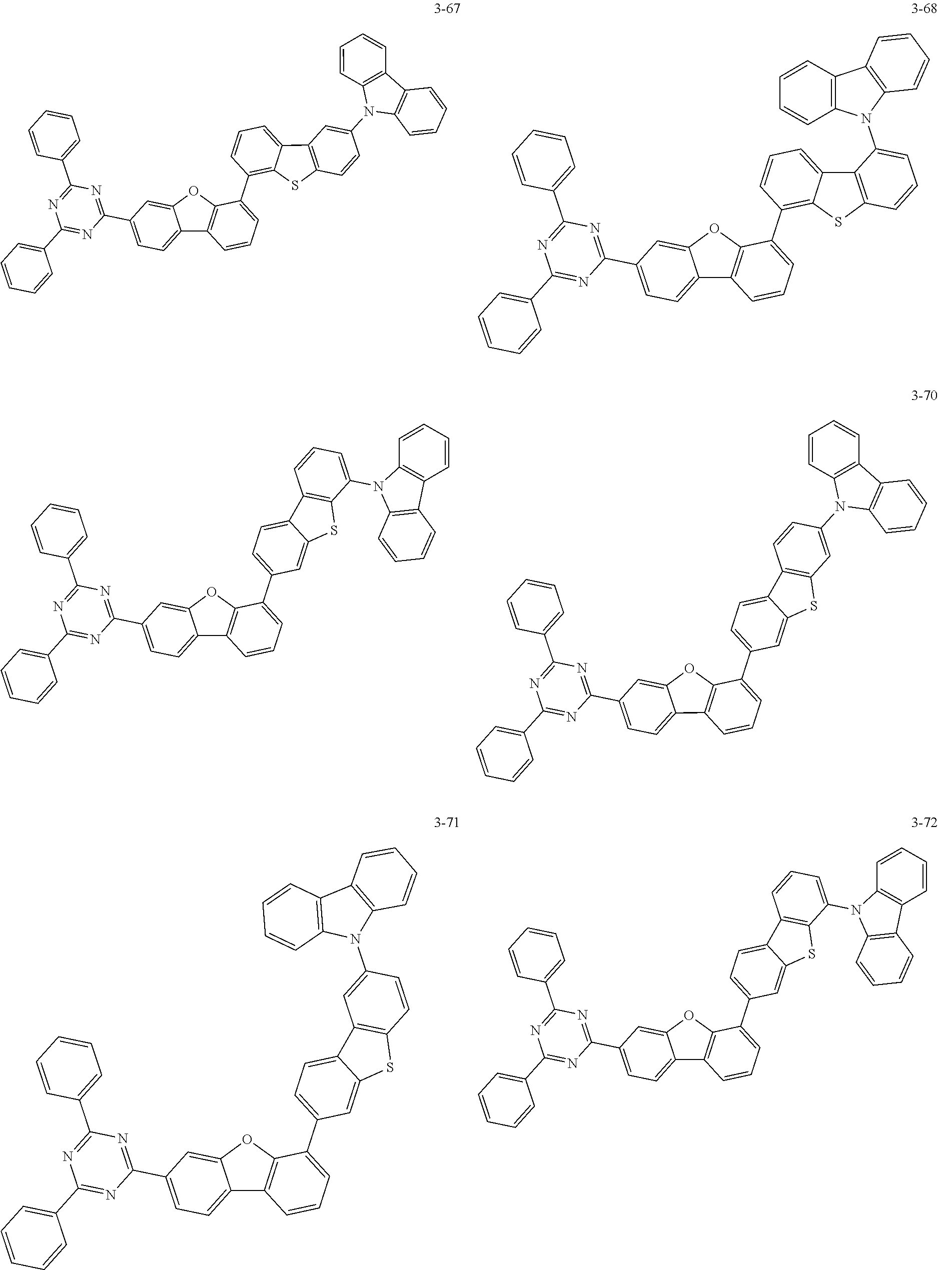

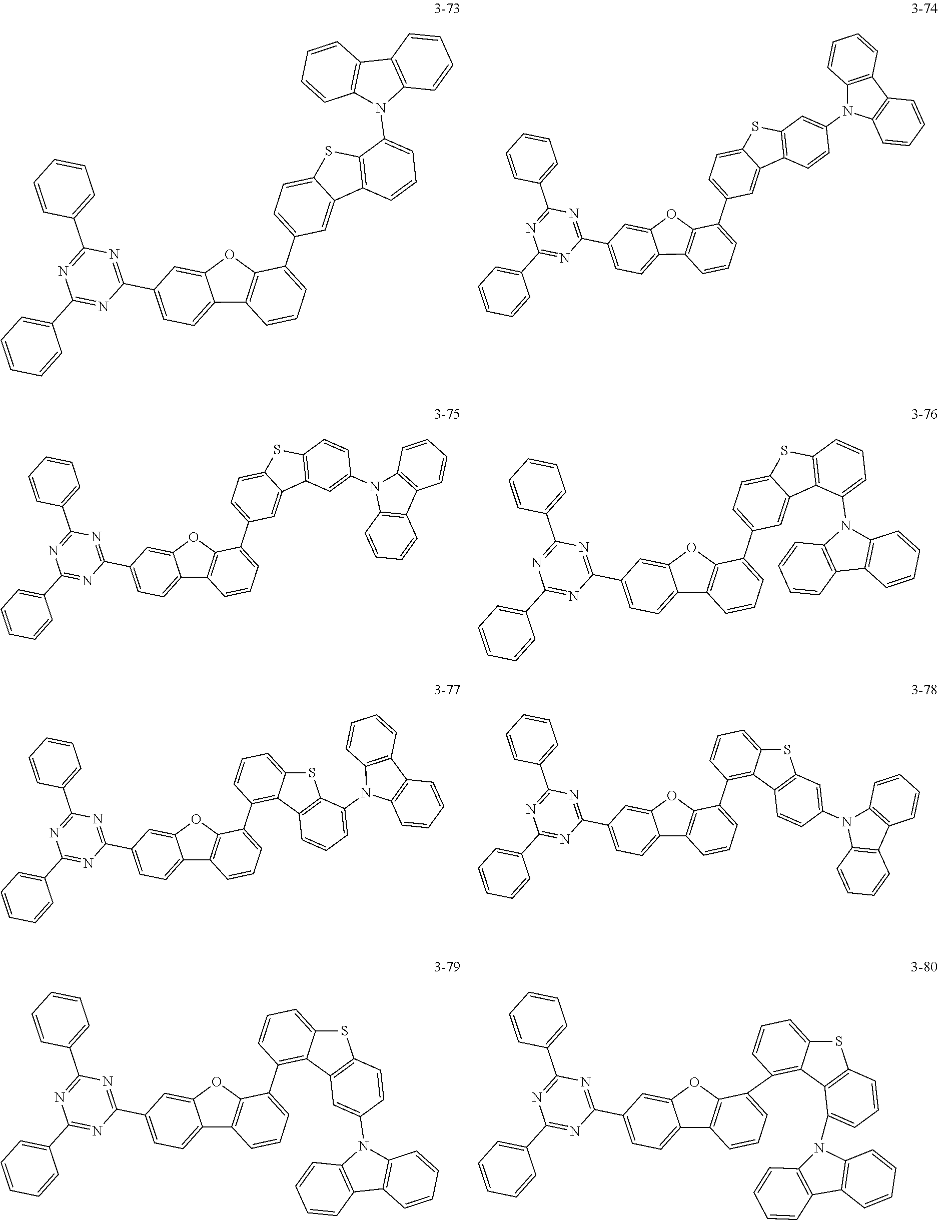

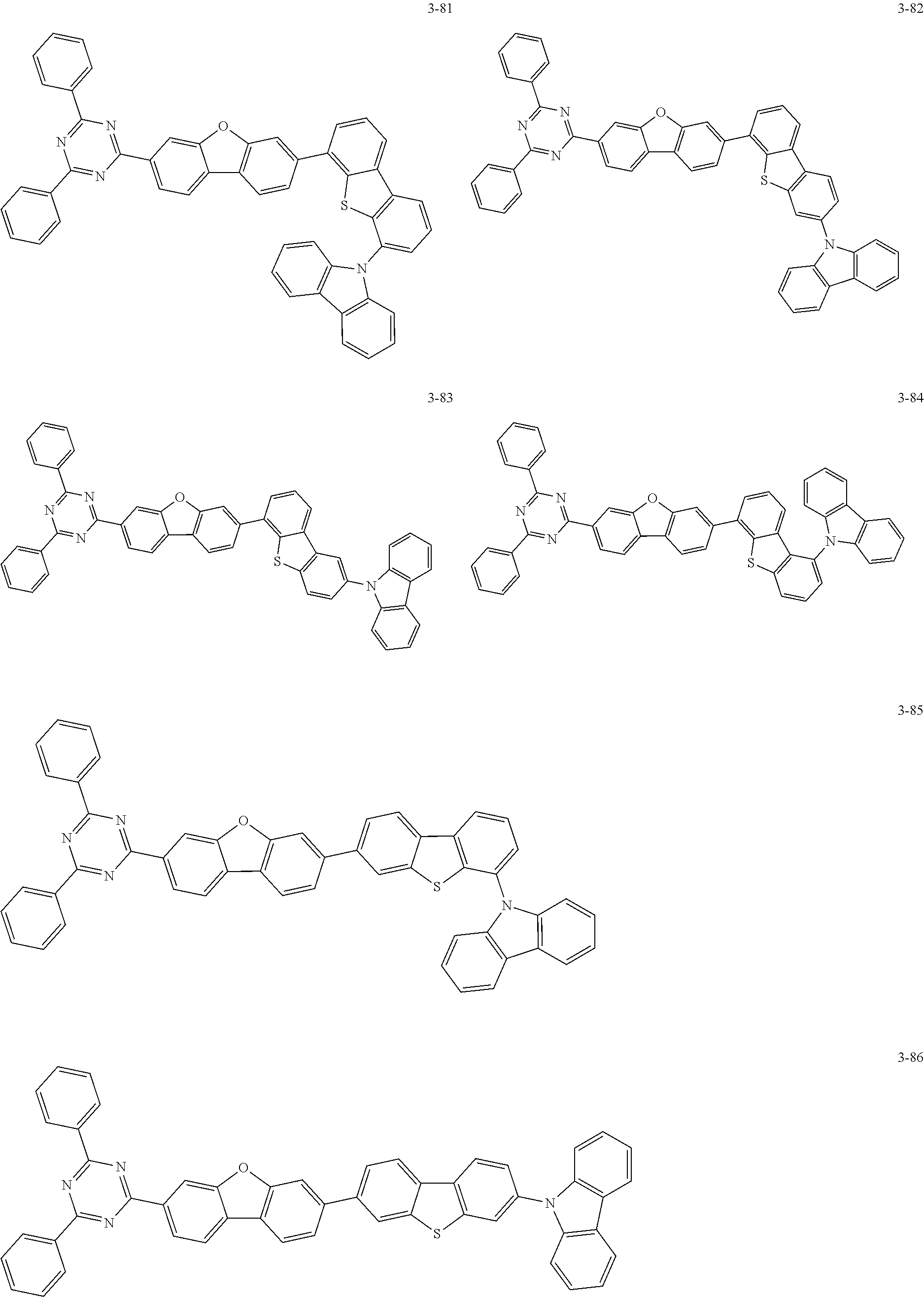

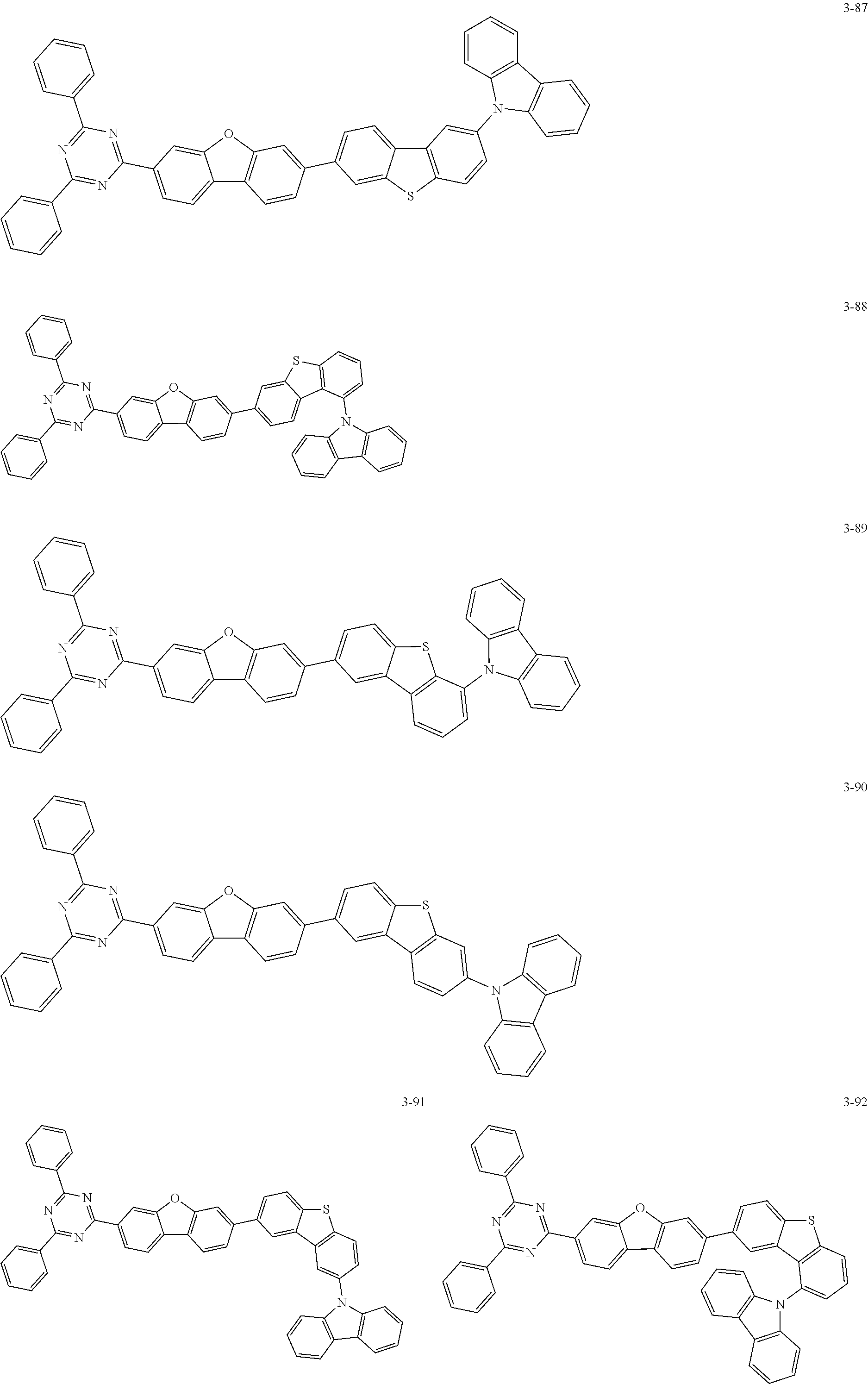

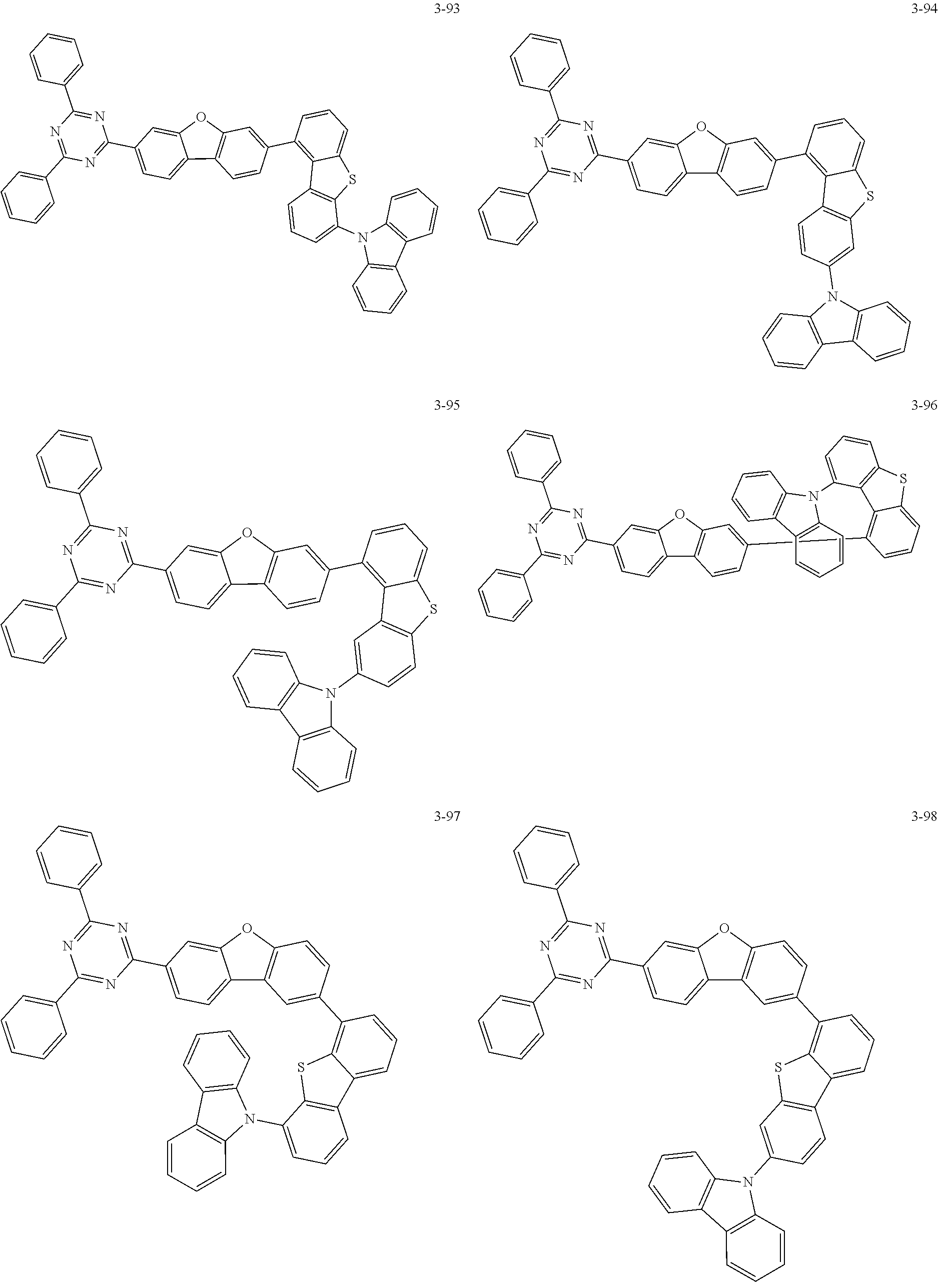

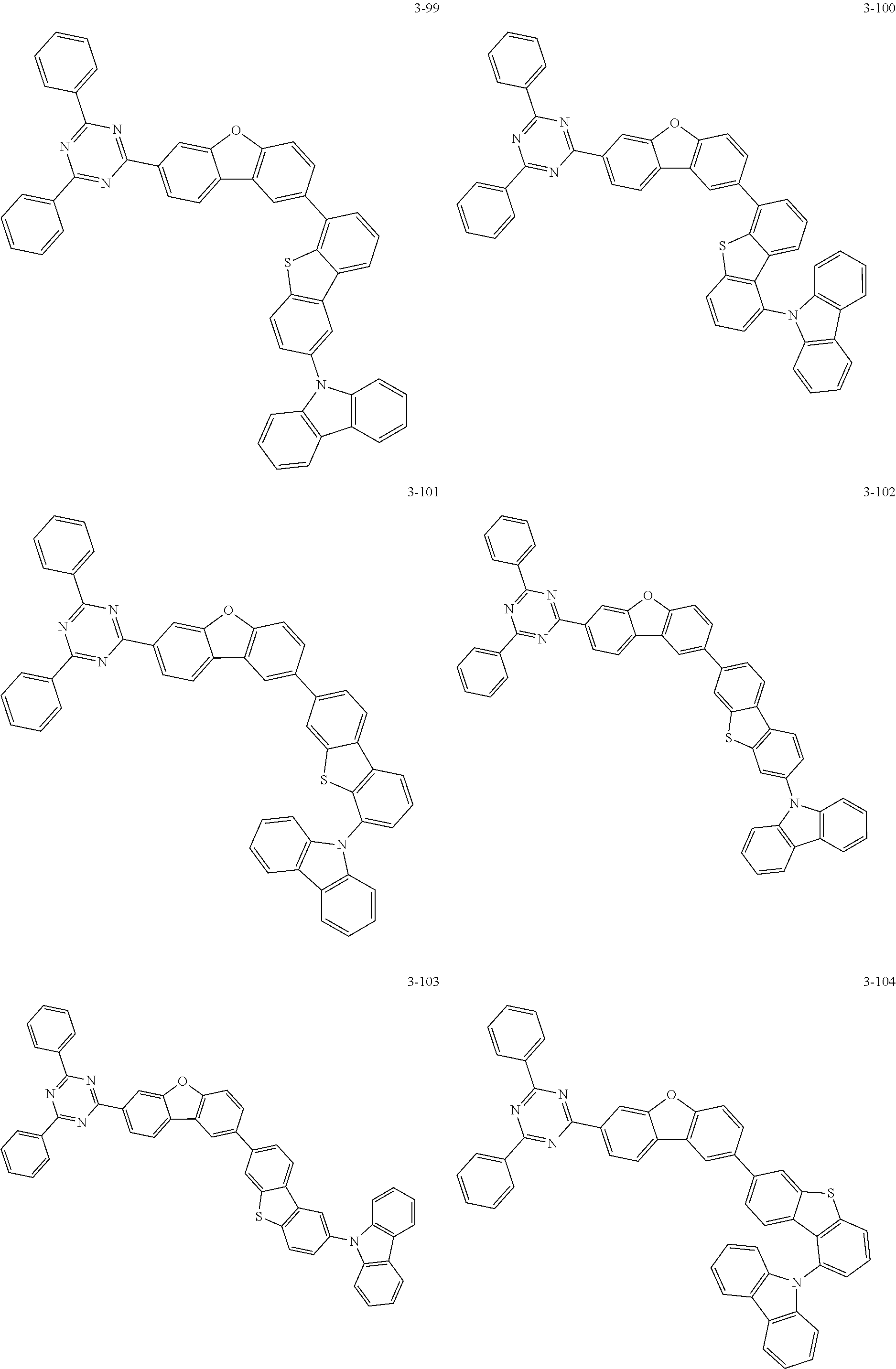

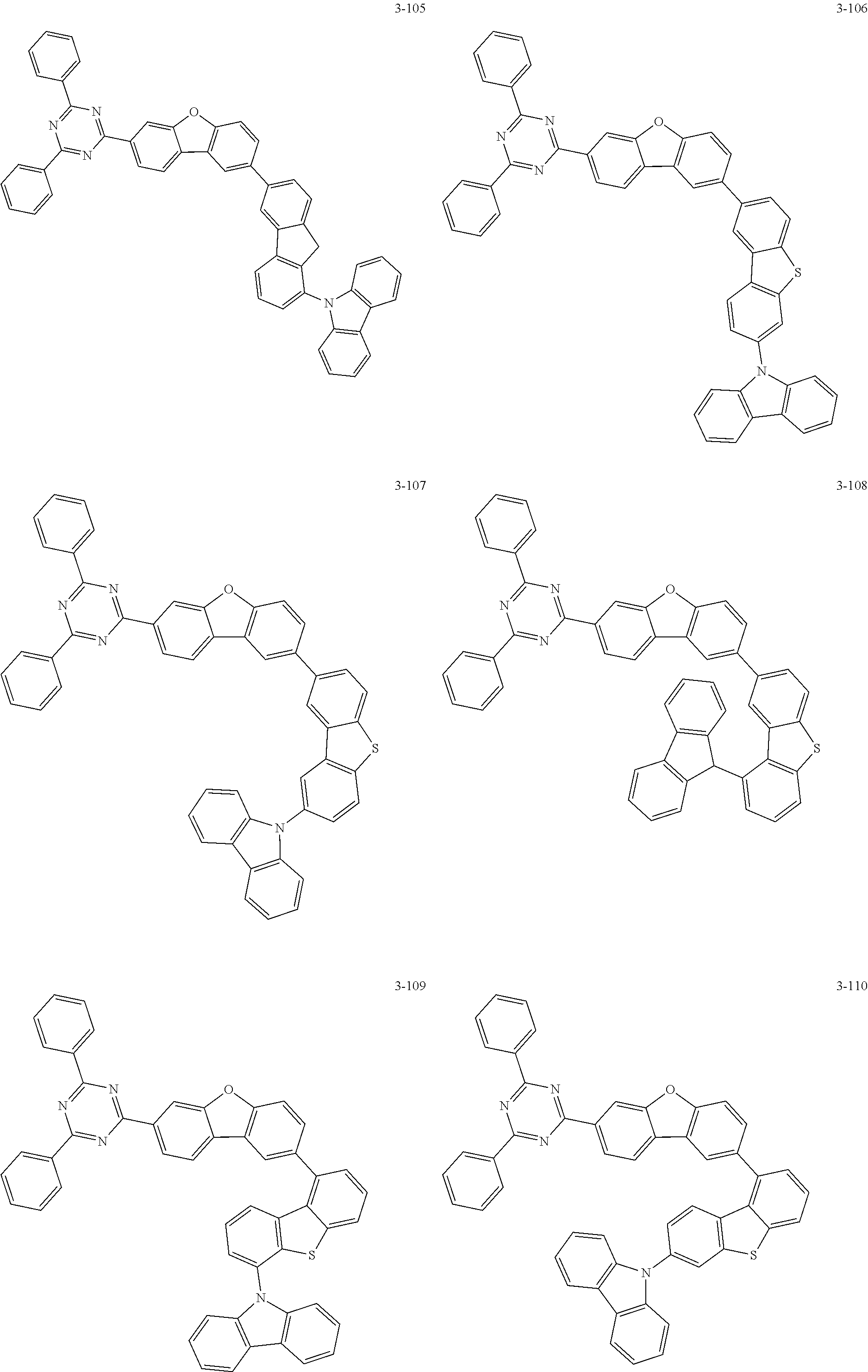

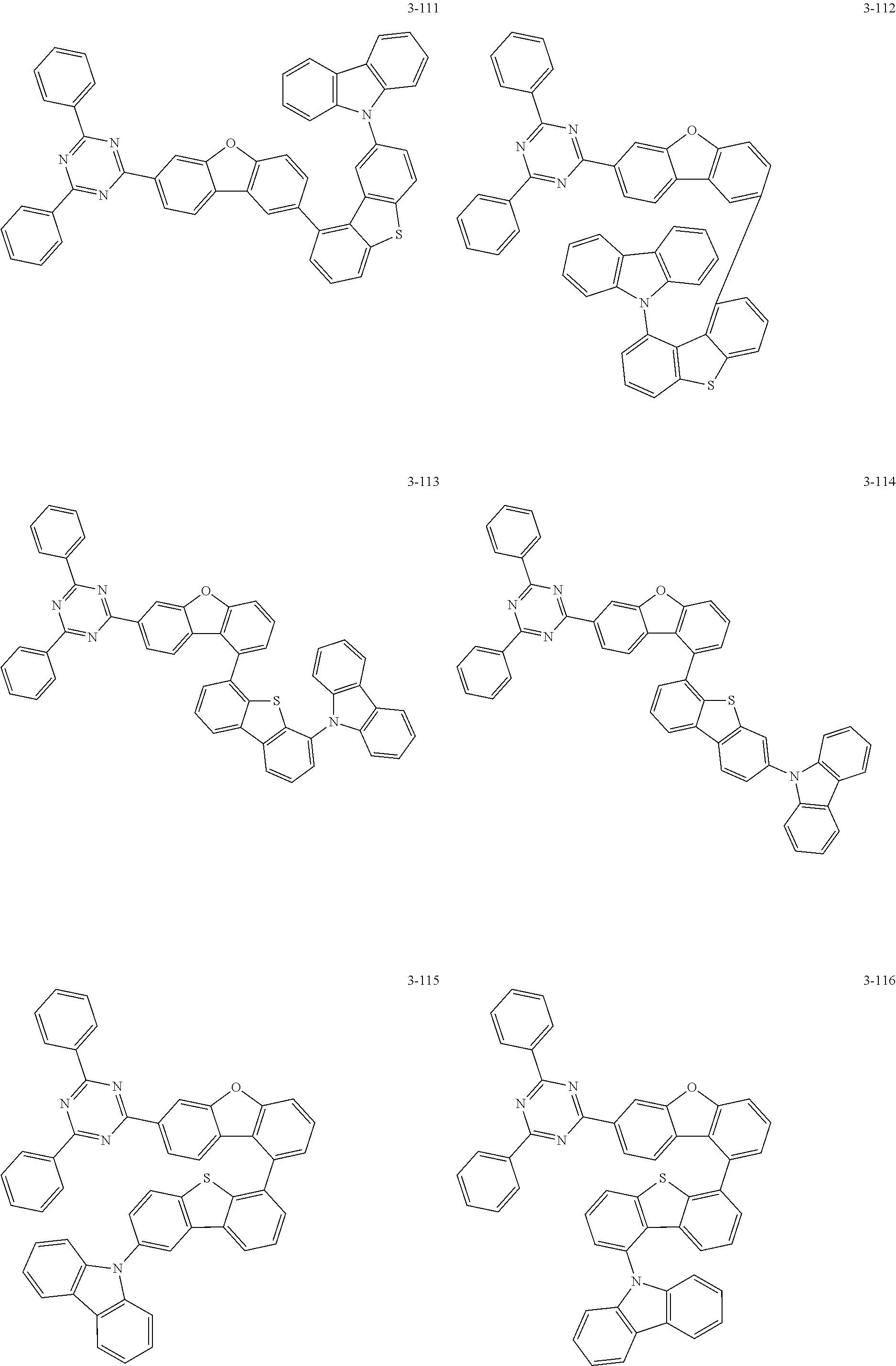

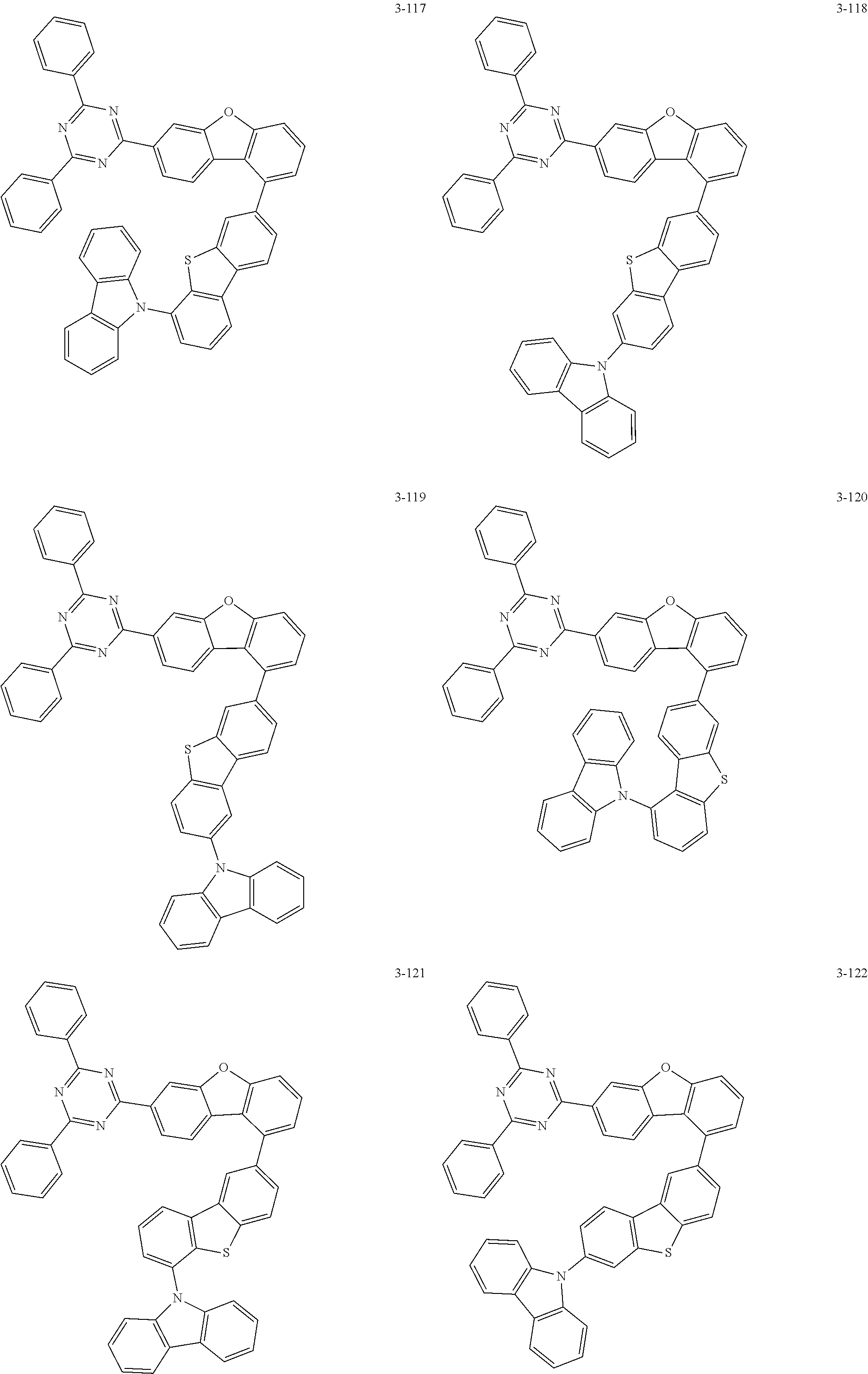

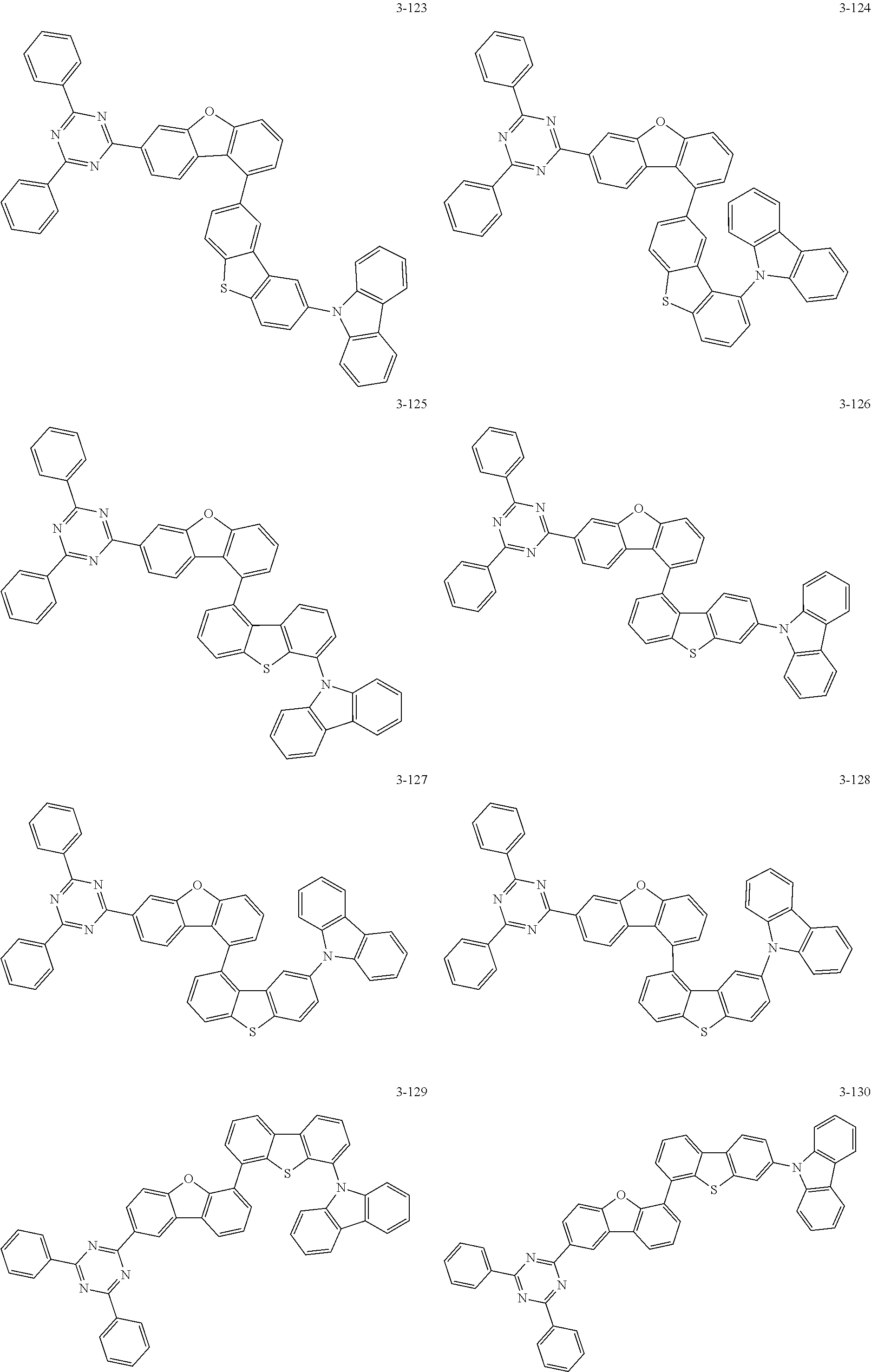

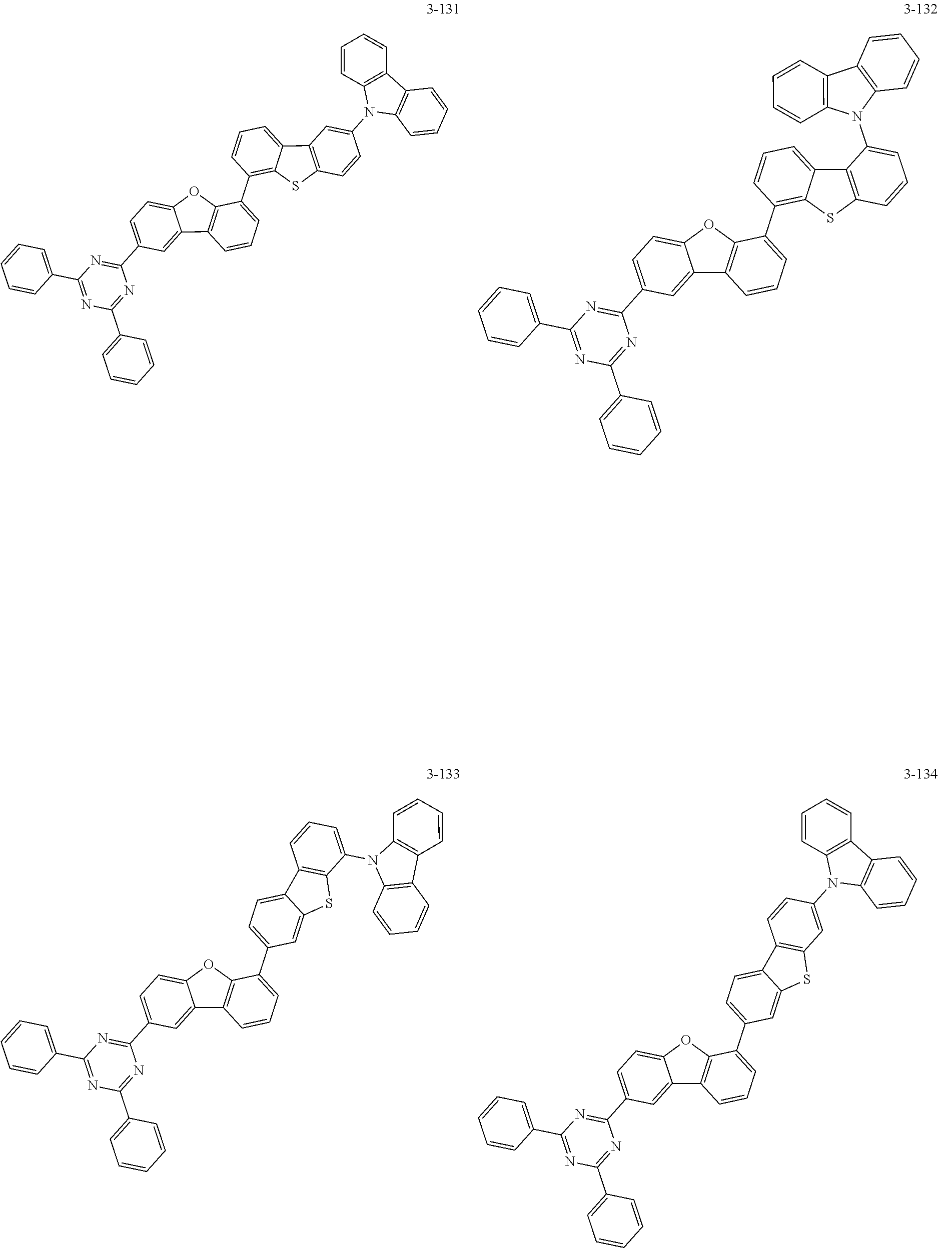 [SMILES: c1ccc(-c2nc(-c3ccccc3)nc(-c3ccc4oc5c(-c6cccc7c6sc6ccc(-n8c9ccccc9c9ccccc98)cc67)cccc5c4c3)n2)cc1 ; c1ccc(-c2nc(-c3ccccc3)nc(-c3ccc4oc5c(-c6cccc7c6sc6cccc(-n8c9ccccc9c9ccccc98)c67)cccc5c4c3)n2)cc1 ; c1ccc(-c2nc(-c3ccccc3)nc(-c3ccc4oc5c(-c6ccc7c(c6)sc6c(-n8c9ccccc9c9ccccc98)cccc67)cccc5c4c3)n2)cc1 ; c1ccc(-c2nc(-c3ccccc3)nc(-c3ccc4oc5c(-c6ccc7c(c6)sc6cc(-n8c9ccccc9c9ccccc98)ccc67)cccc5c4c3)n2)cc1]

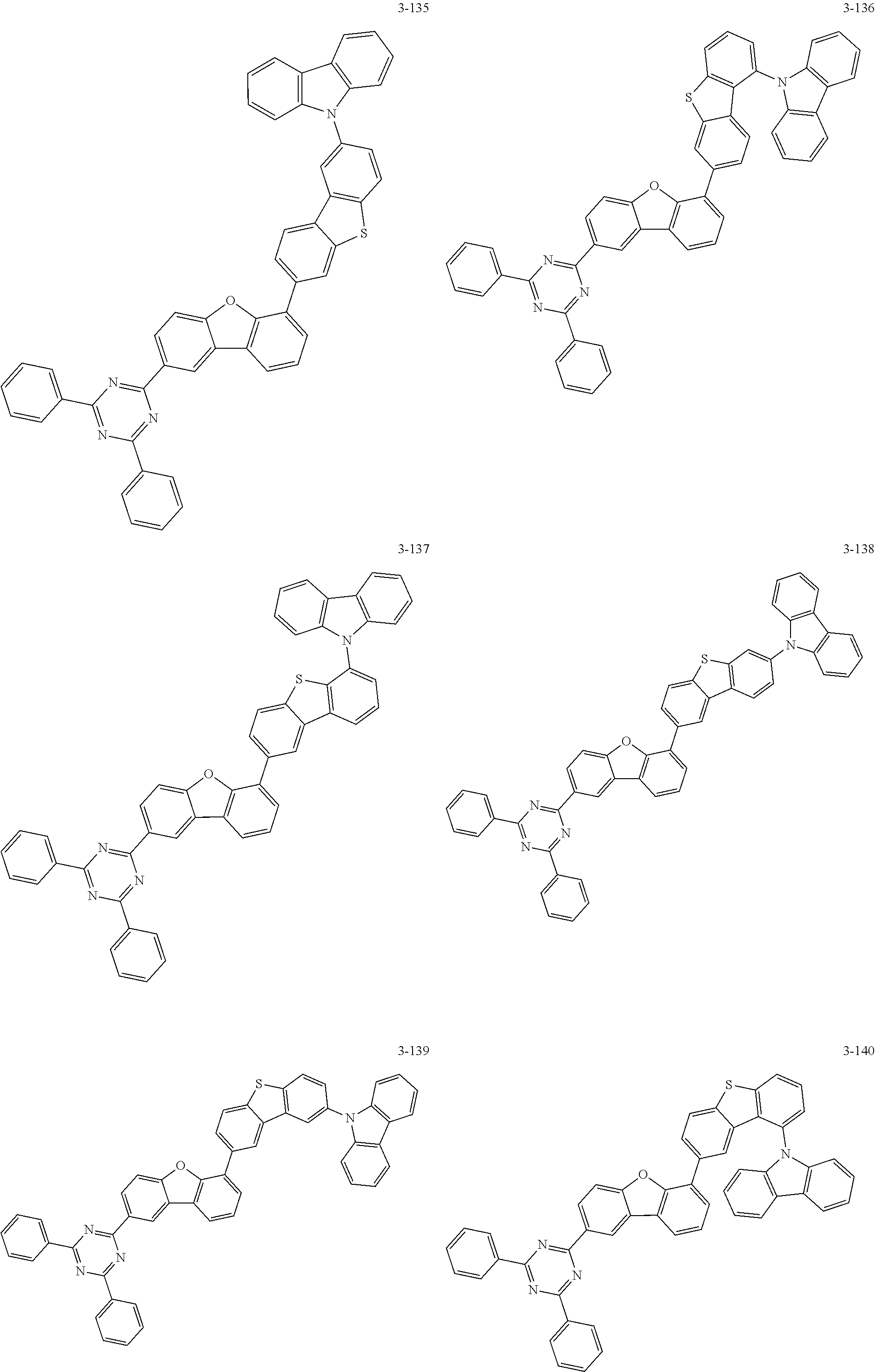

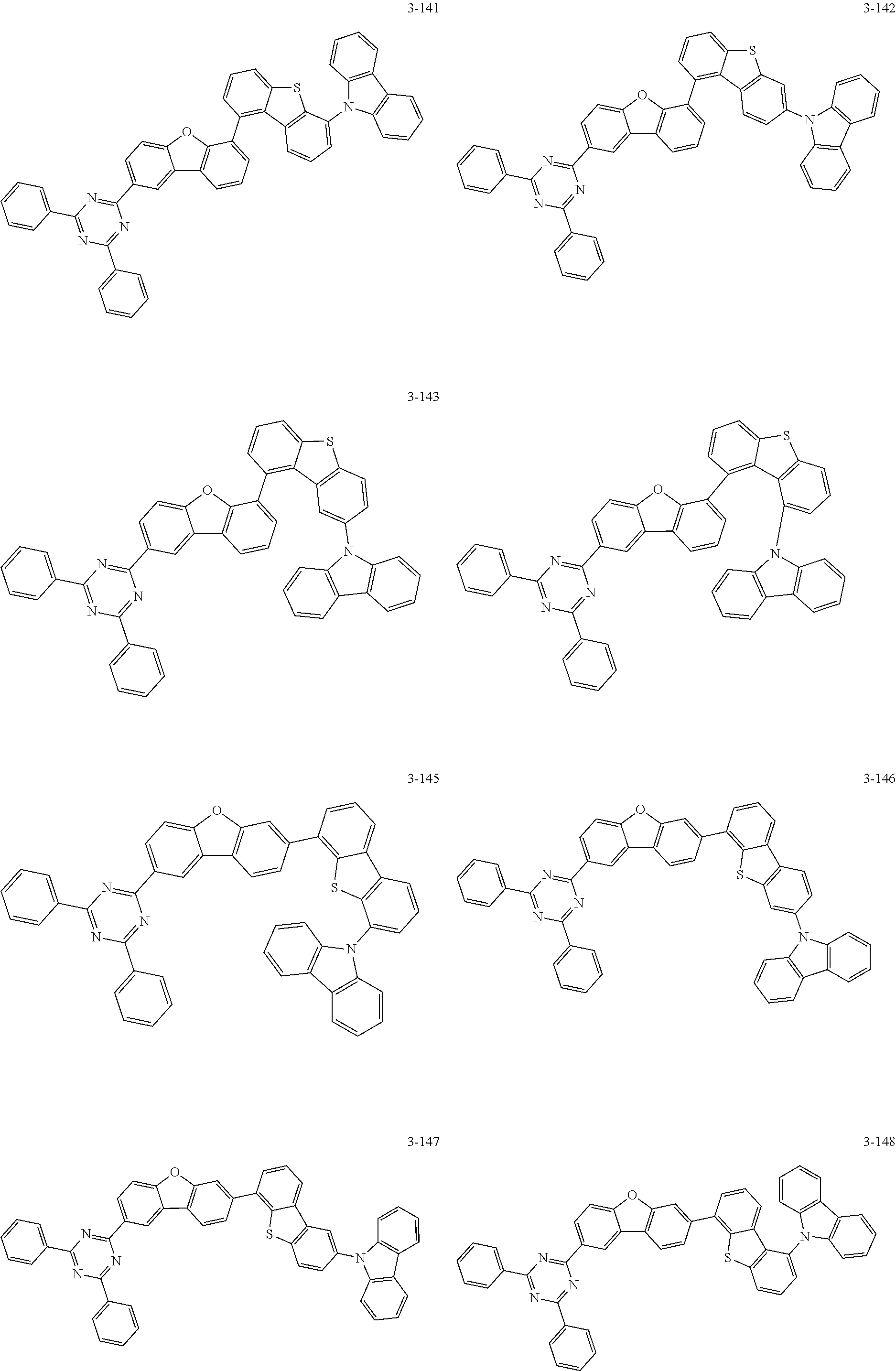

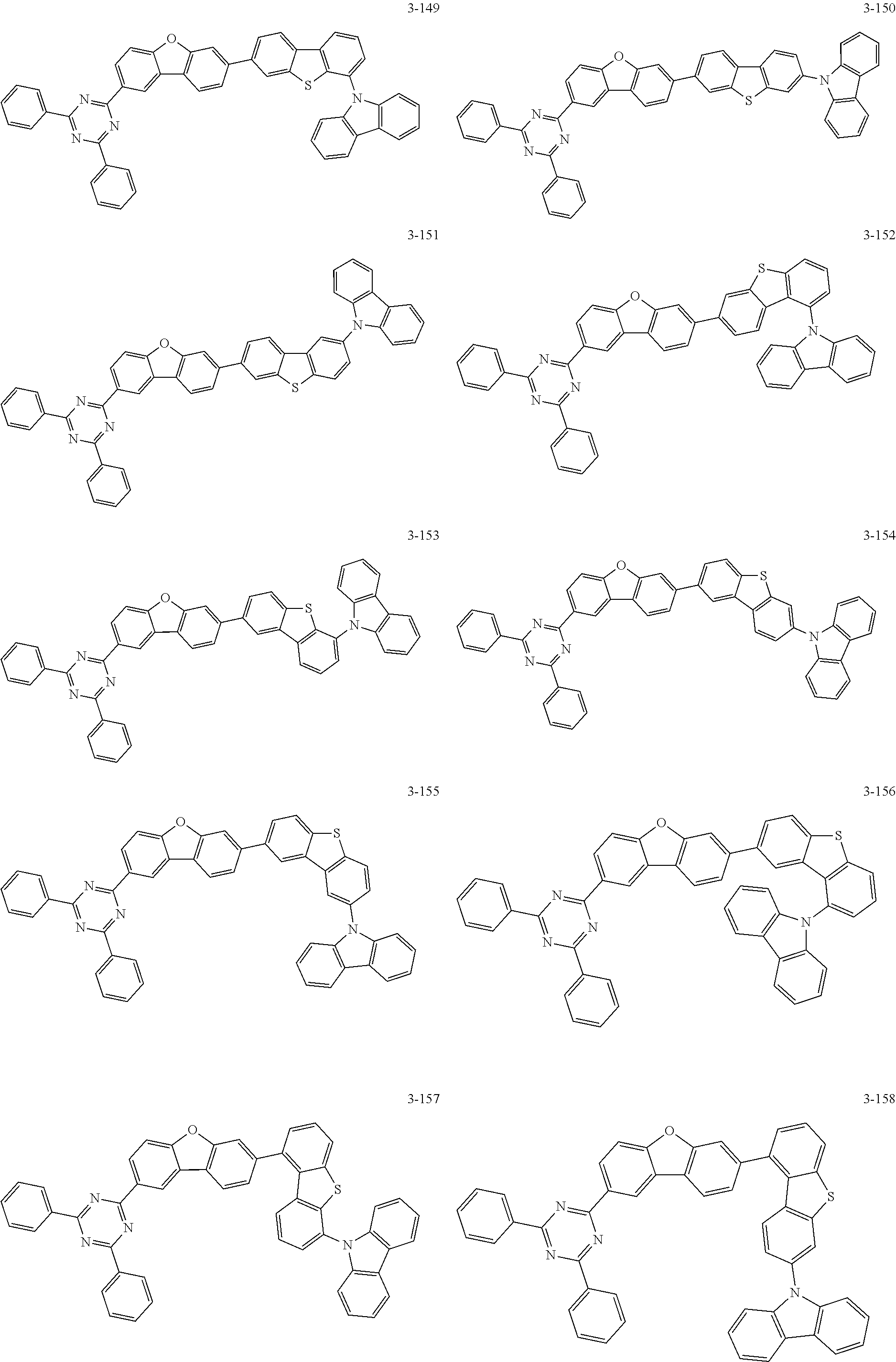

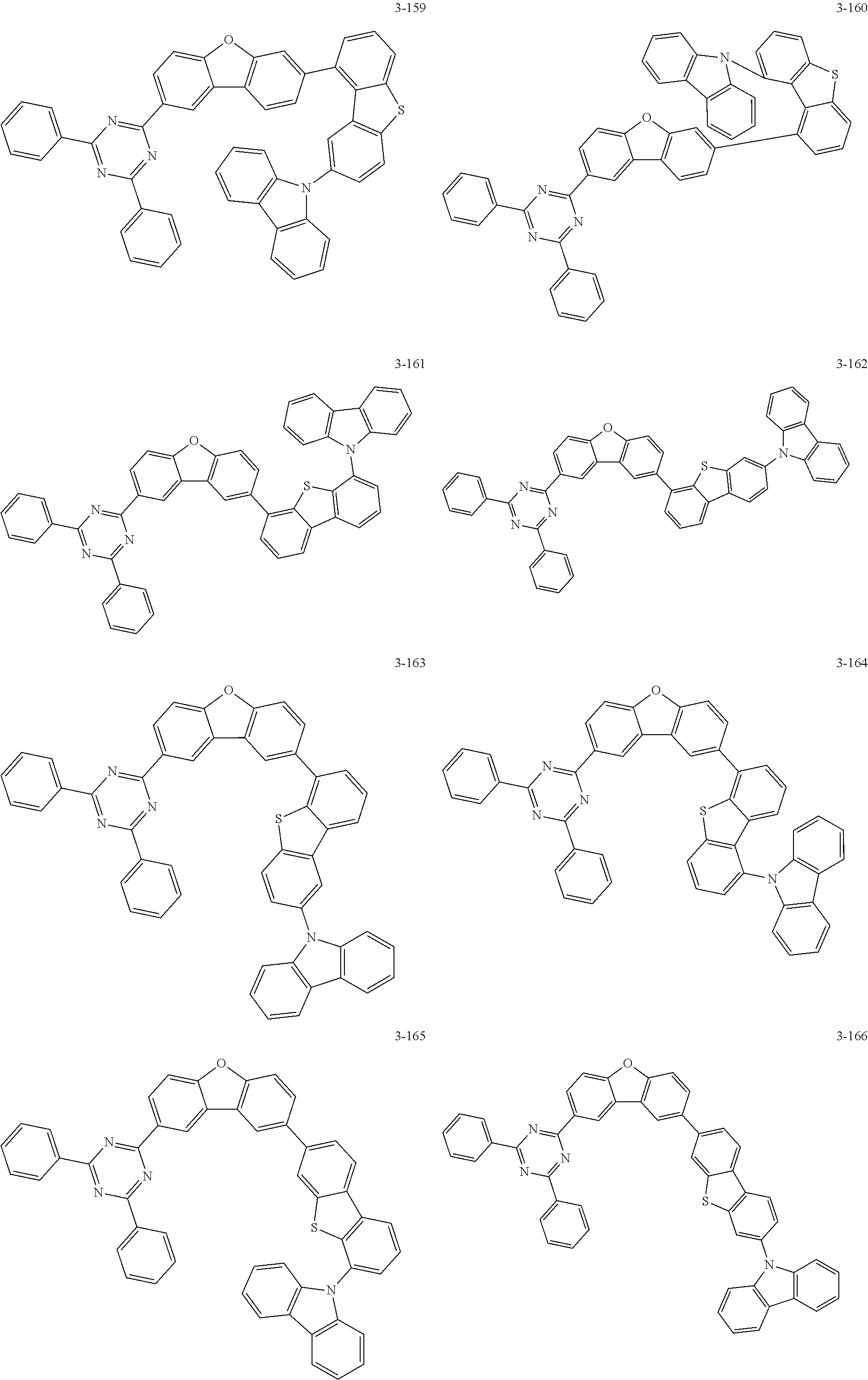

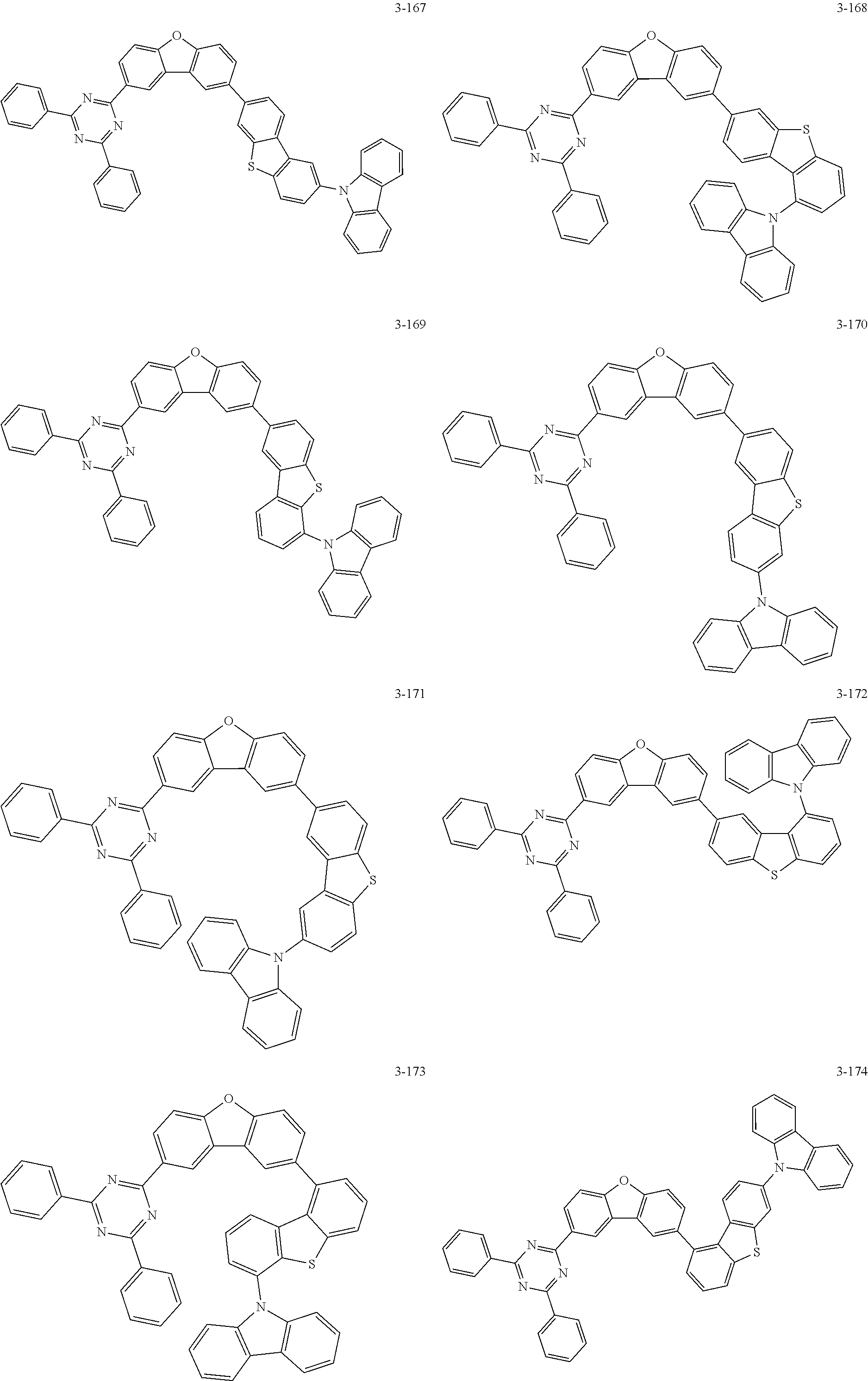

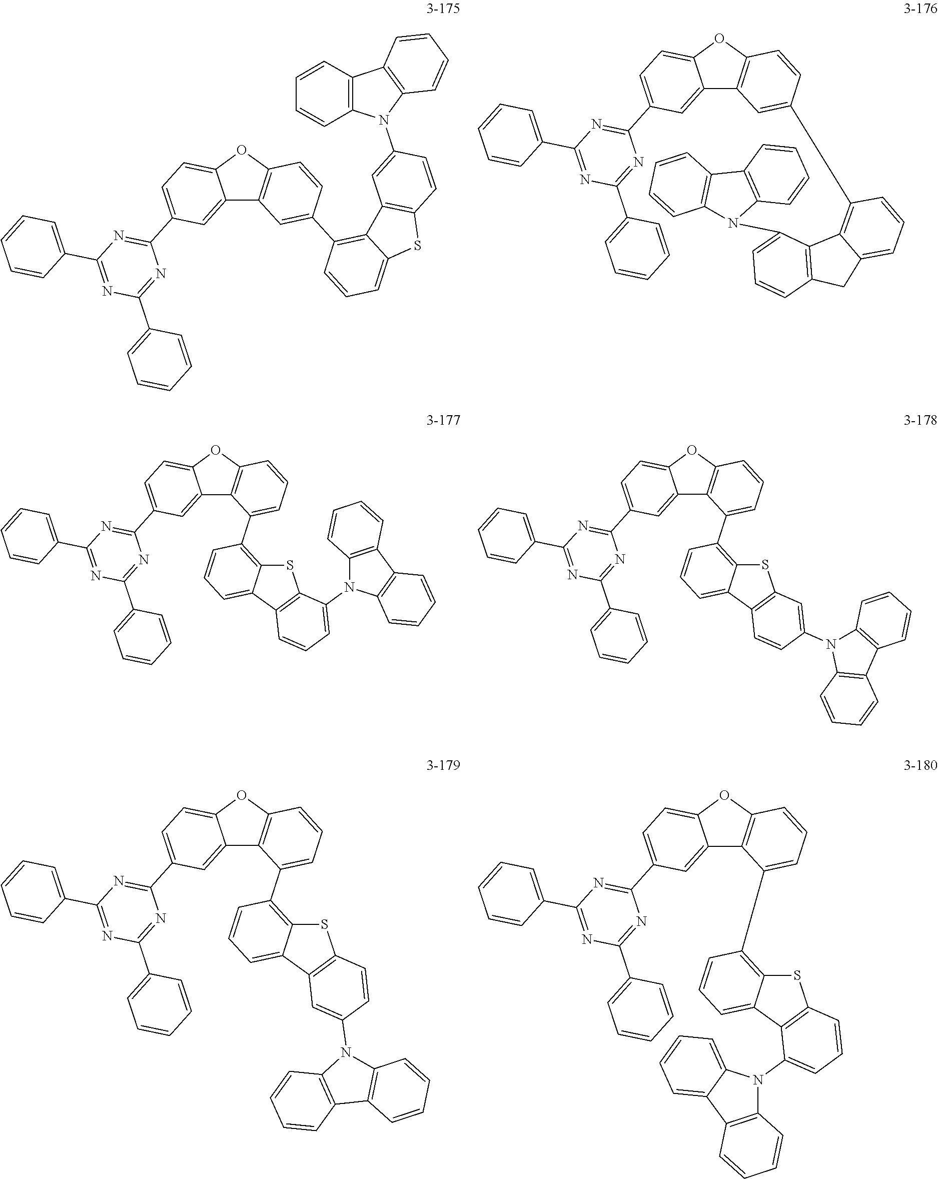

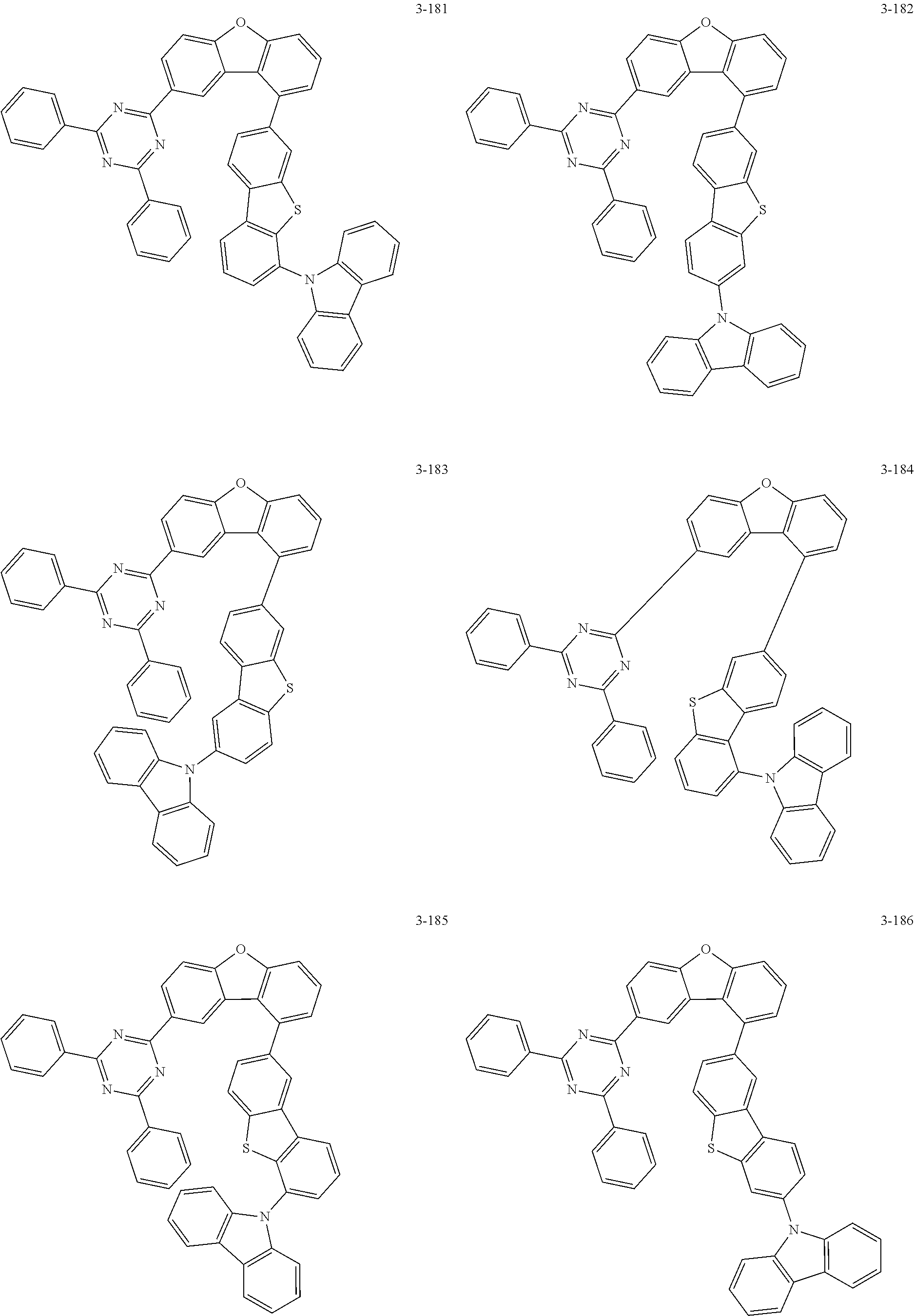

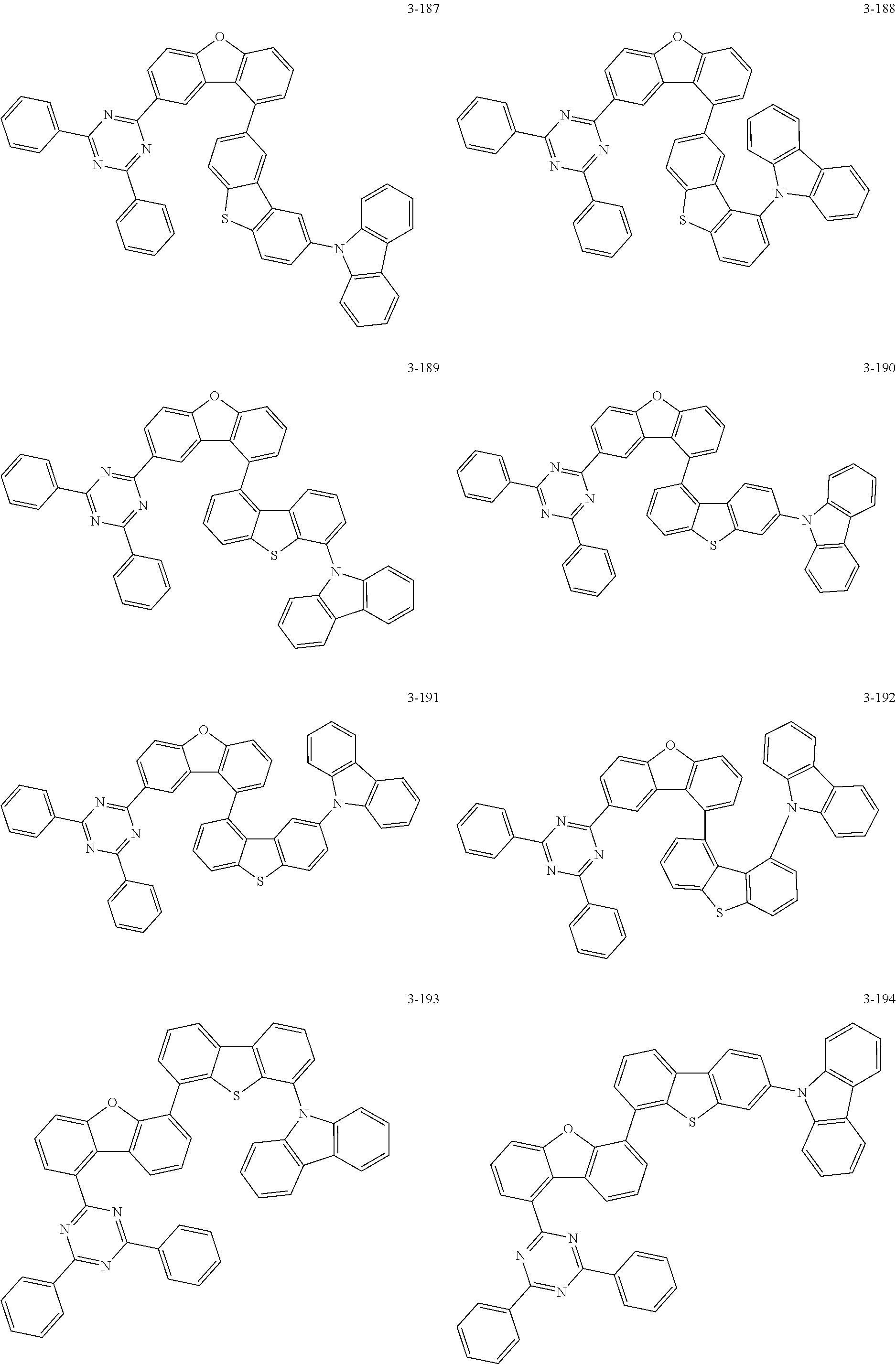

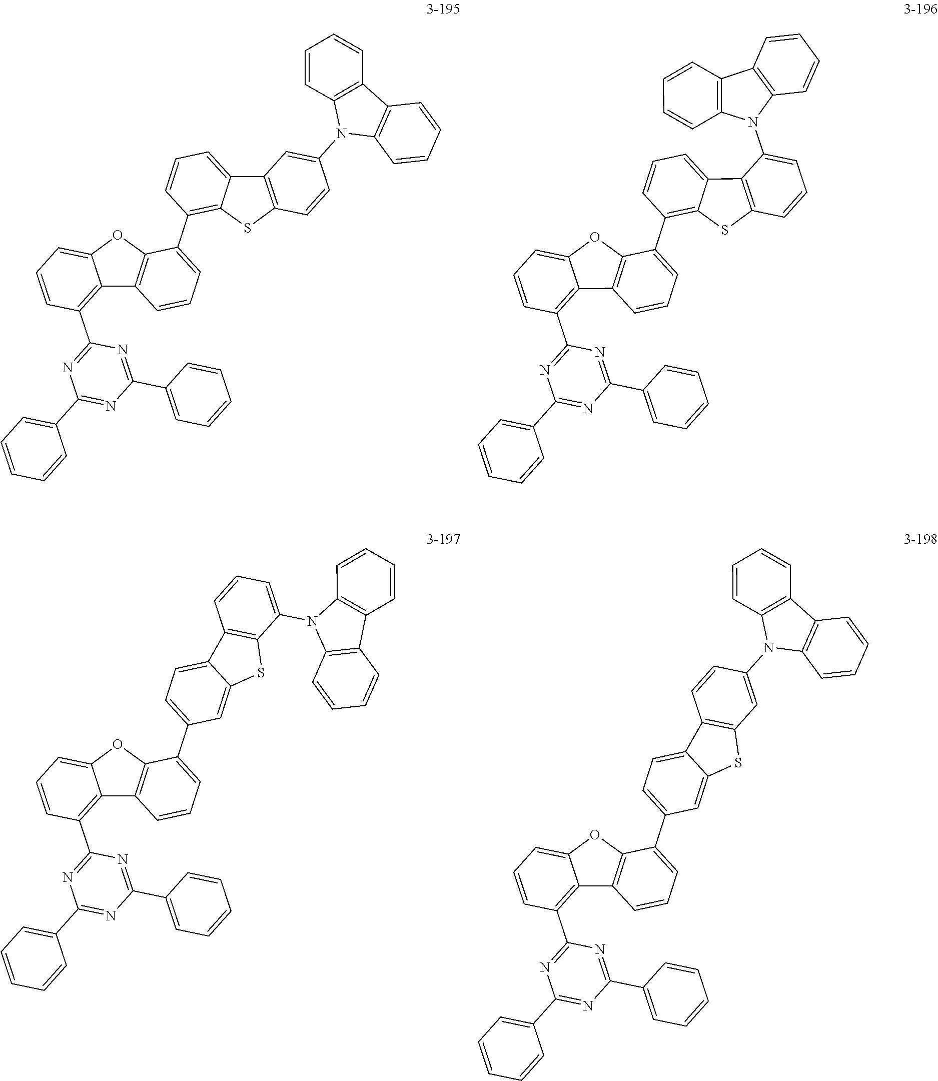 [SMILES: c1ccc(-c2nc(-c3ccccc3)nc(-c3cccc4oc5c(-c6cccc7c6sc6ccc(-n8c9ccccc9c9ccccc98)cc67)cccc5c34)n2)cc1 ; c1ccc(-c2nc(-c3ccccc3)nc(-c3cccc4oc5c(-c6cccc7c6sc6cccc(-n8c9ccccc9c9ccccc98)c67)cccc5c34)n2)cc1 ; c1ccc(-c2nc(-c3ccccc3)nc(-c3cccc4oc5c(-c6ccc7c(c6)sc6c(-n8c9ccccc9c9ccccc98)cccc67)cccc5c34)n2)cc1 ; c1ccc(-c2nc(-c3ccccc3)nc(-c3cccc4oc5c(-c6ccc7c(c6)sc6cc(-n8c9ccccc9c9ccccc98)ccc67)cccc5c34)n2)cc1]

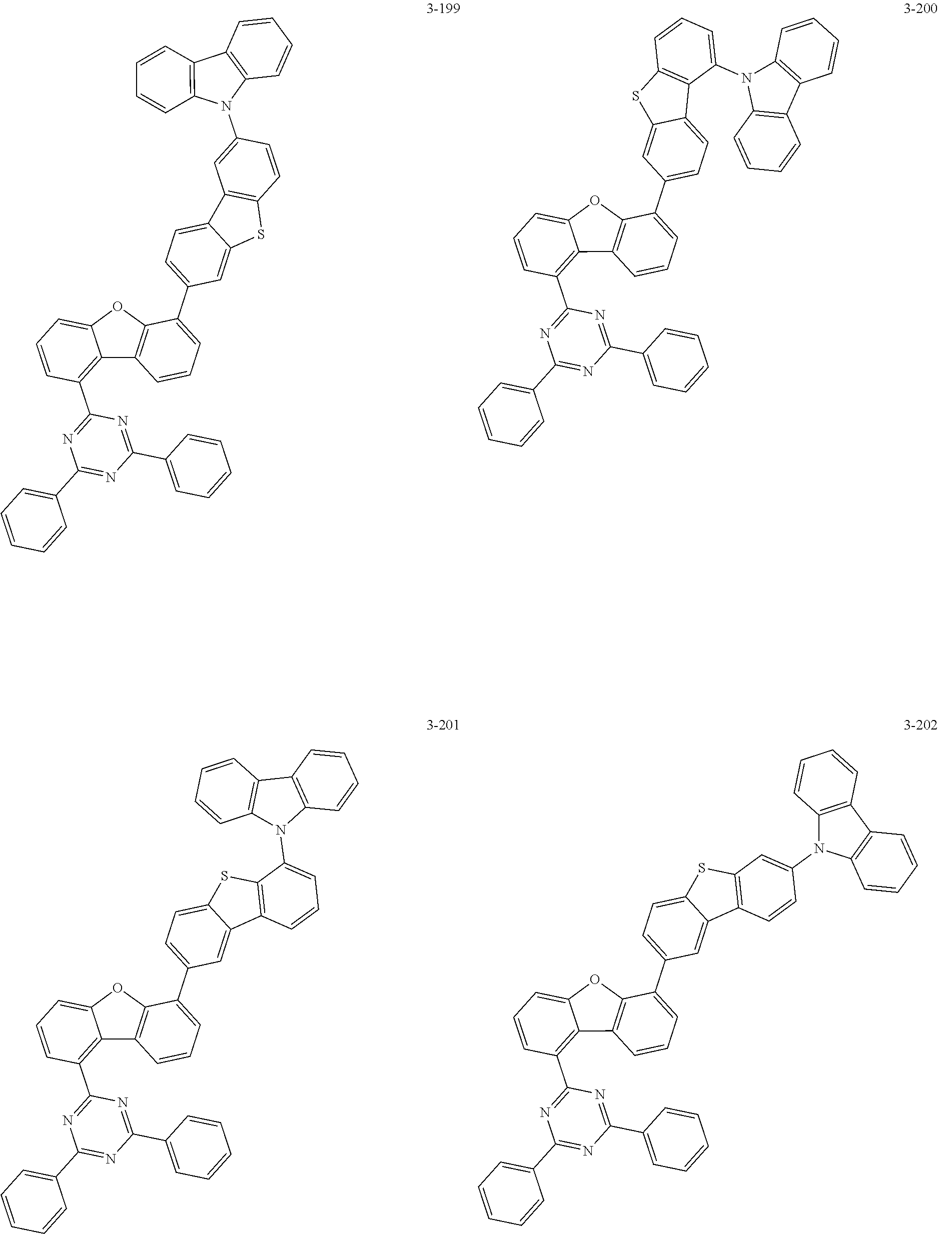 [SMILES: c1ccc(-c2nc(-c3ccccc3)nc(-c3cccc4oc5c(-c6ccc7c(c6)sc6ccc(-n8c9ccccc9c9ccccc98)cc67)cccc5c34)n2)cc1 ; c1ccc(-c2nc(-c3ccccc3)nc(-c3cccc4oc5c(-c6ccc7c(c6)sc6cccc(-n8c9ccccc9c9ccccc98)c67)cccc5c34)n2)cc1 ; c1ccc(-c2nc(-c3ccccc3)nc(-c3cccc4oc5c(-c6ccc7sc8c(-n9c%10ccccc%10c%10ccccc%109)cccc8c7c6)cccc5c34)n2)cc1 ; c1ccc(-c2nc(-c3ccccc3)nc(-c3cccc4oc5c(-c6ccc7sc8cc(-n9c%10ccccc%10c%10ccccc%109)ccc8c7c6)cccc5c34)n2)cc1]

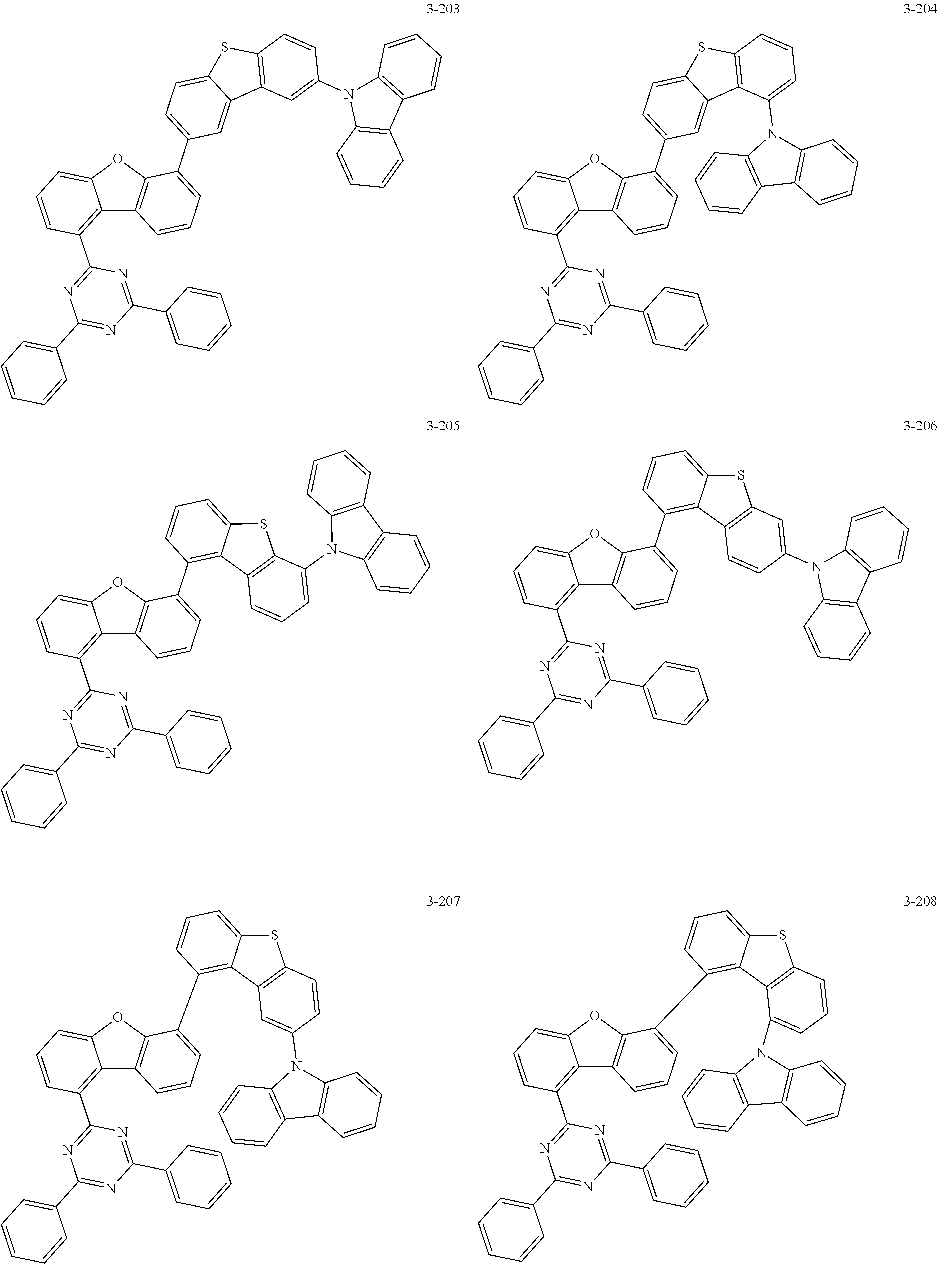

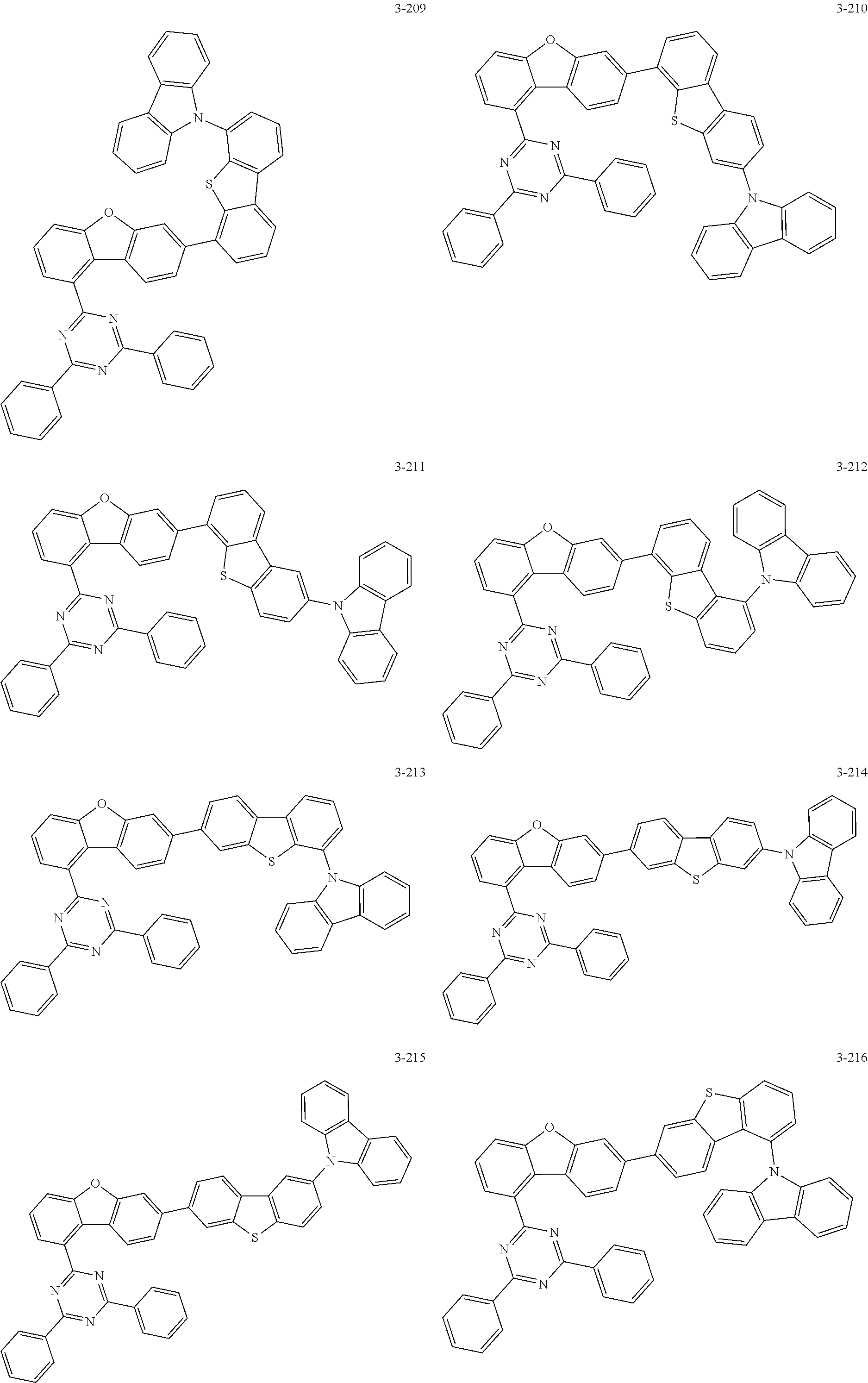

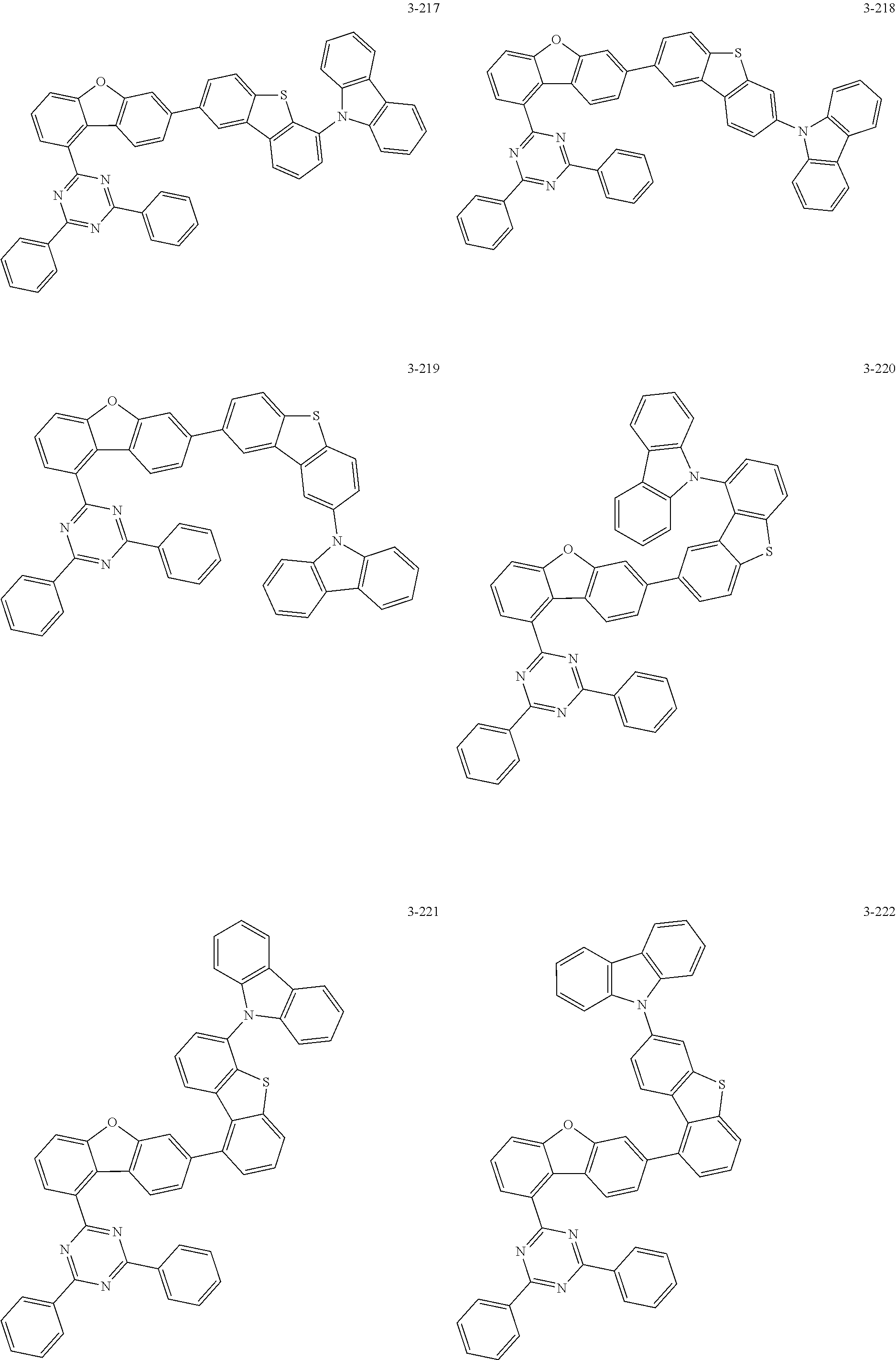

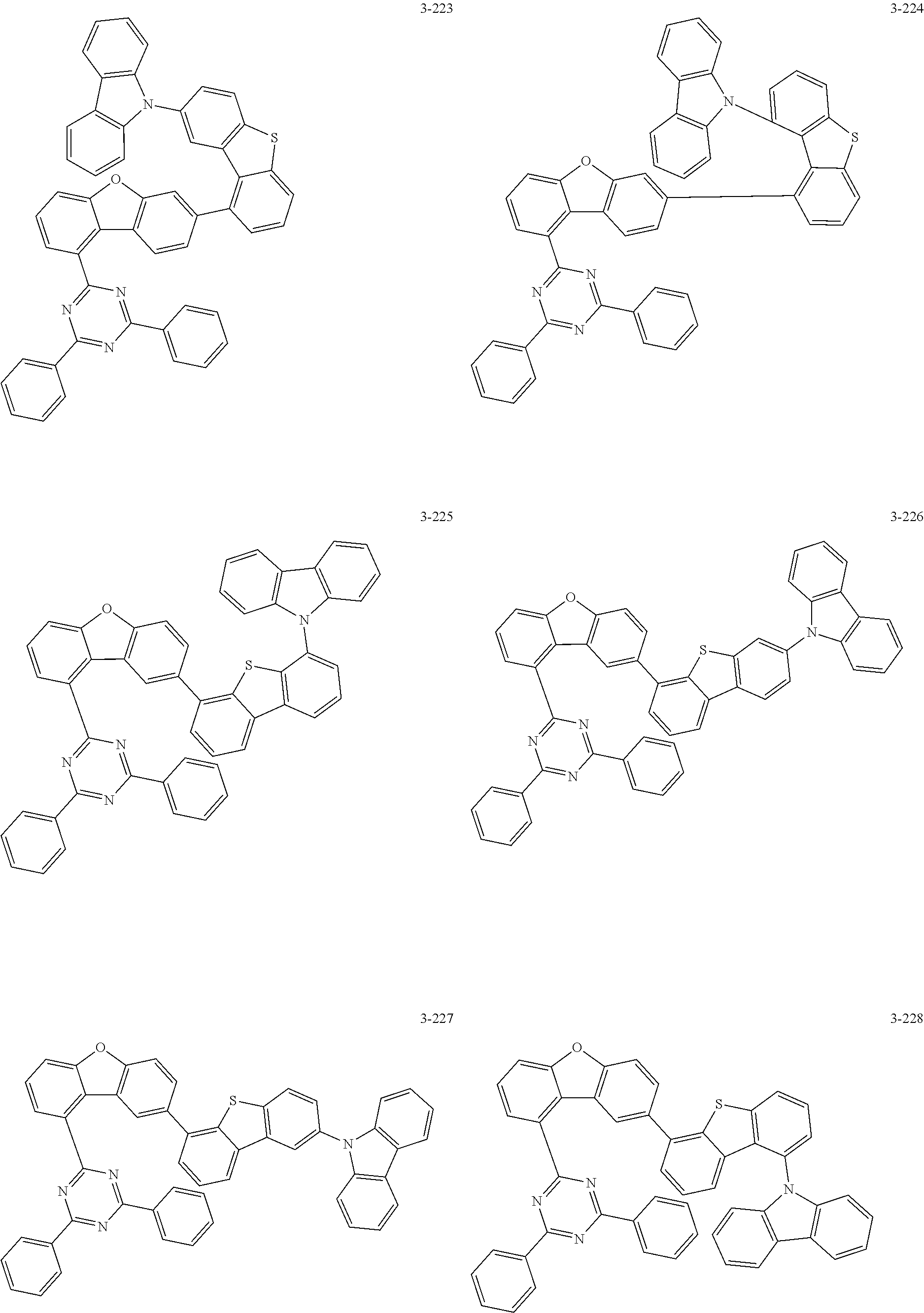

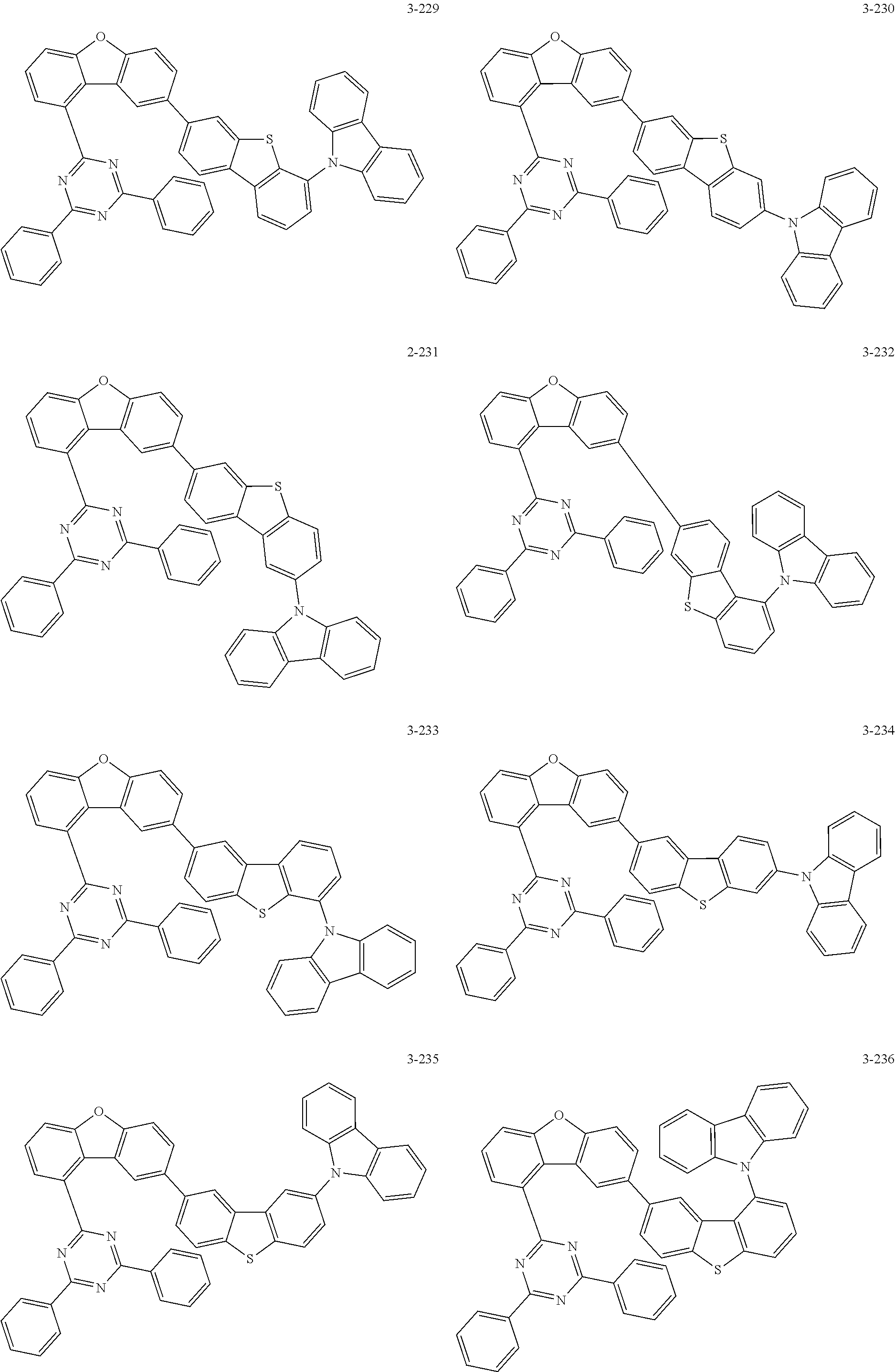

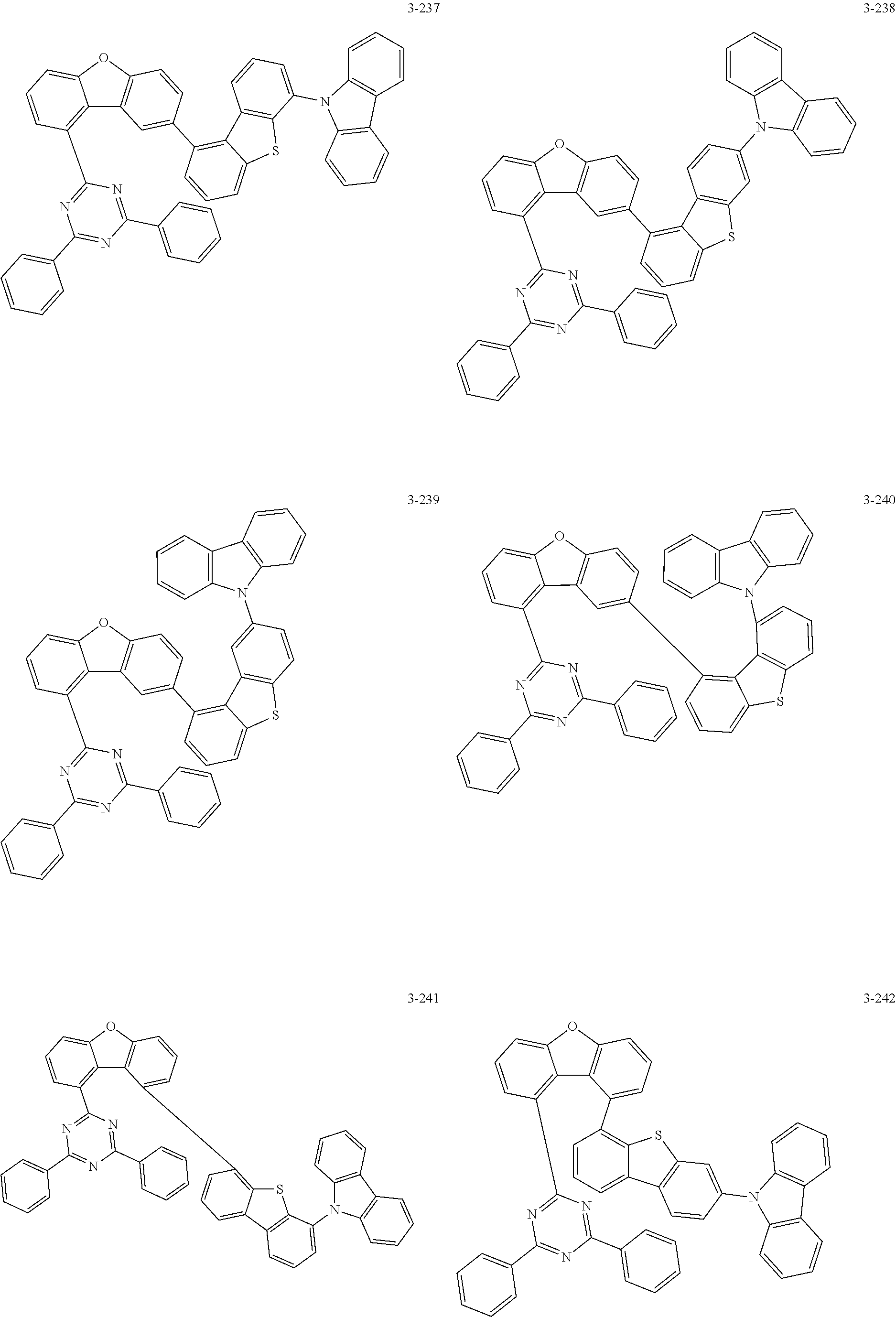

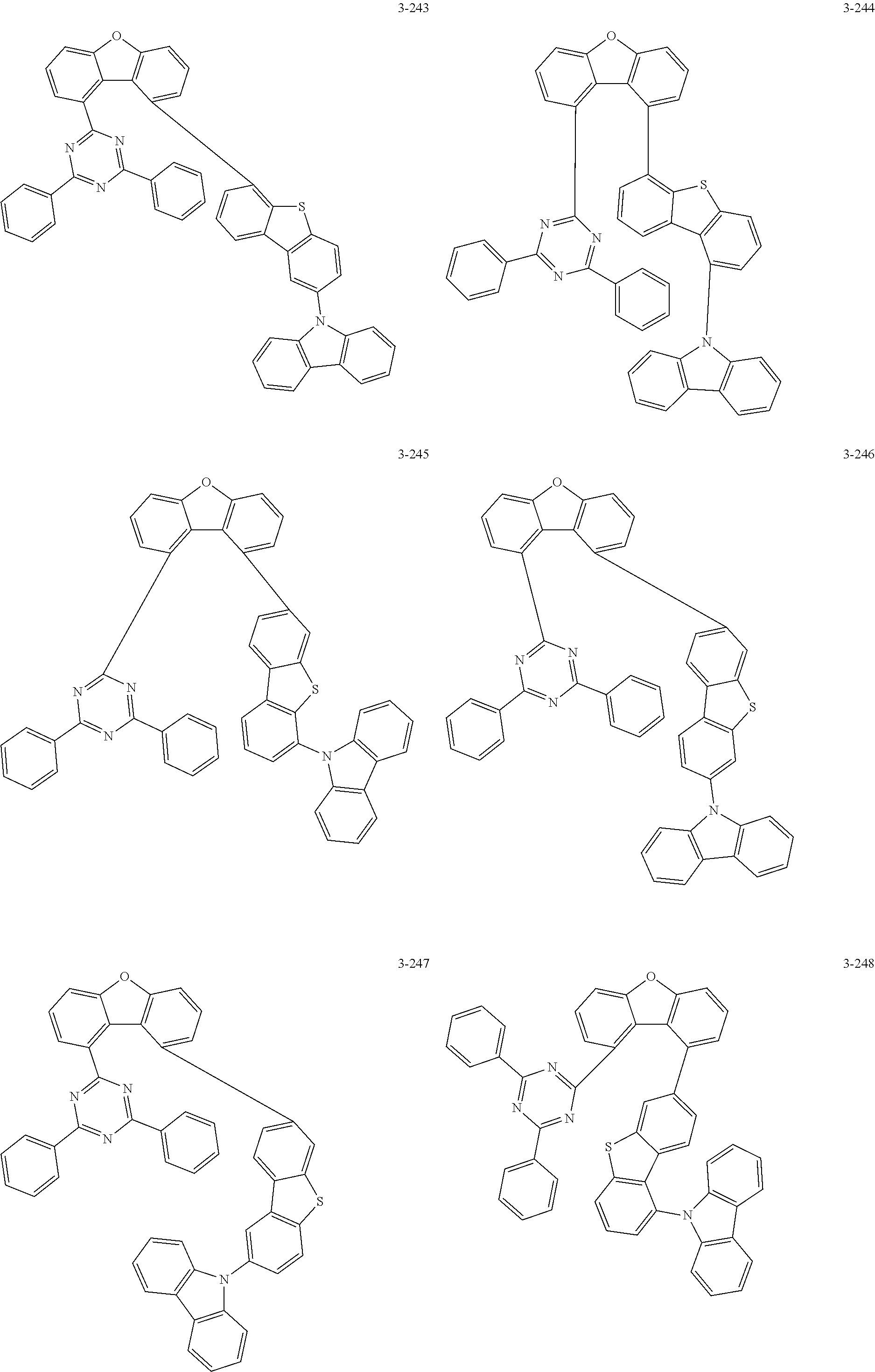

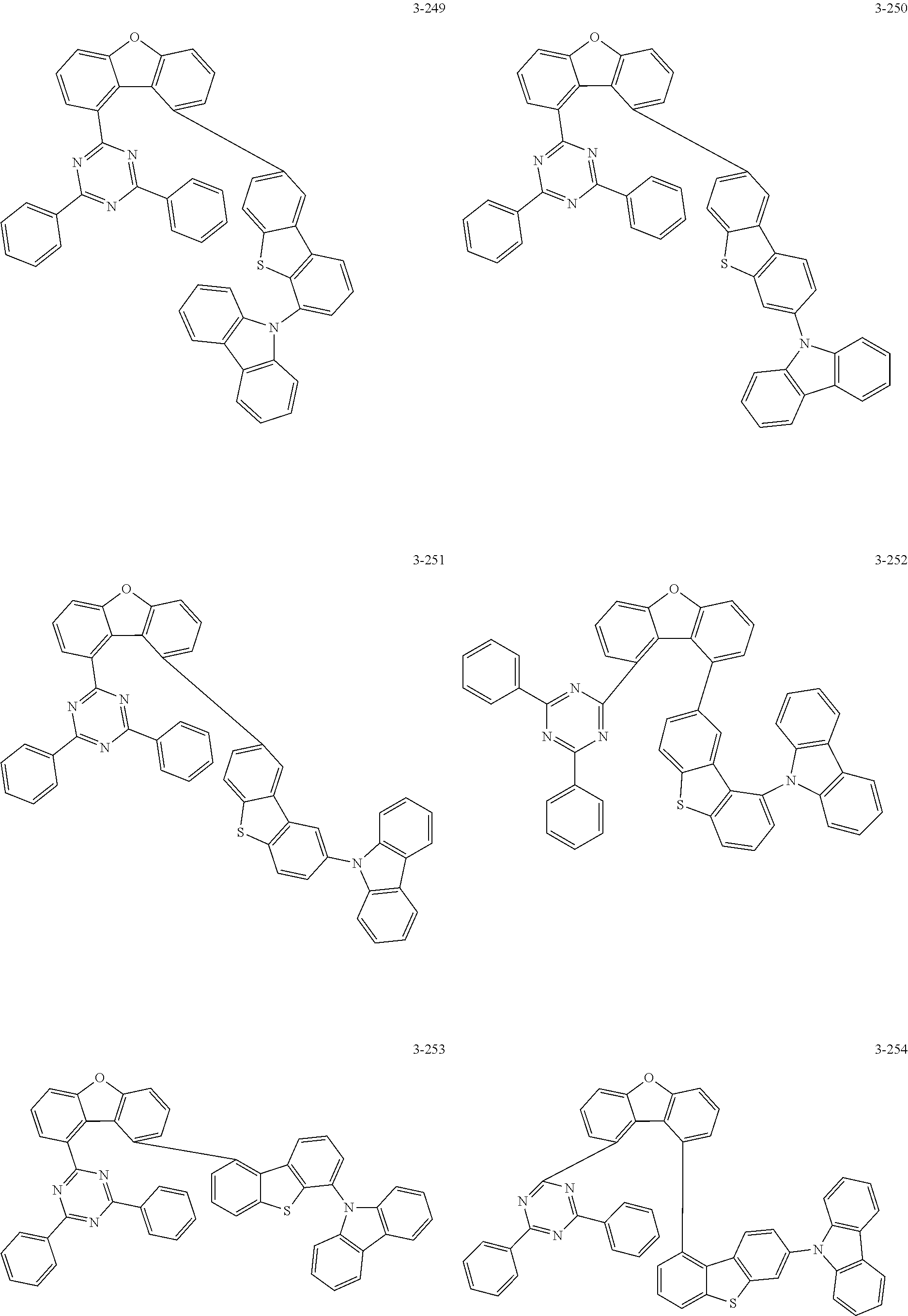

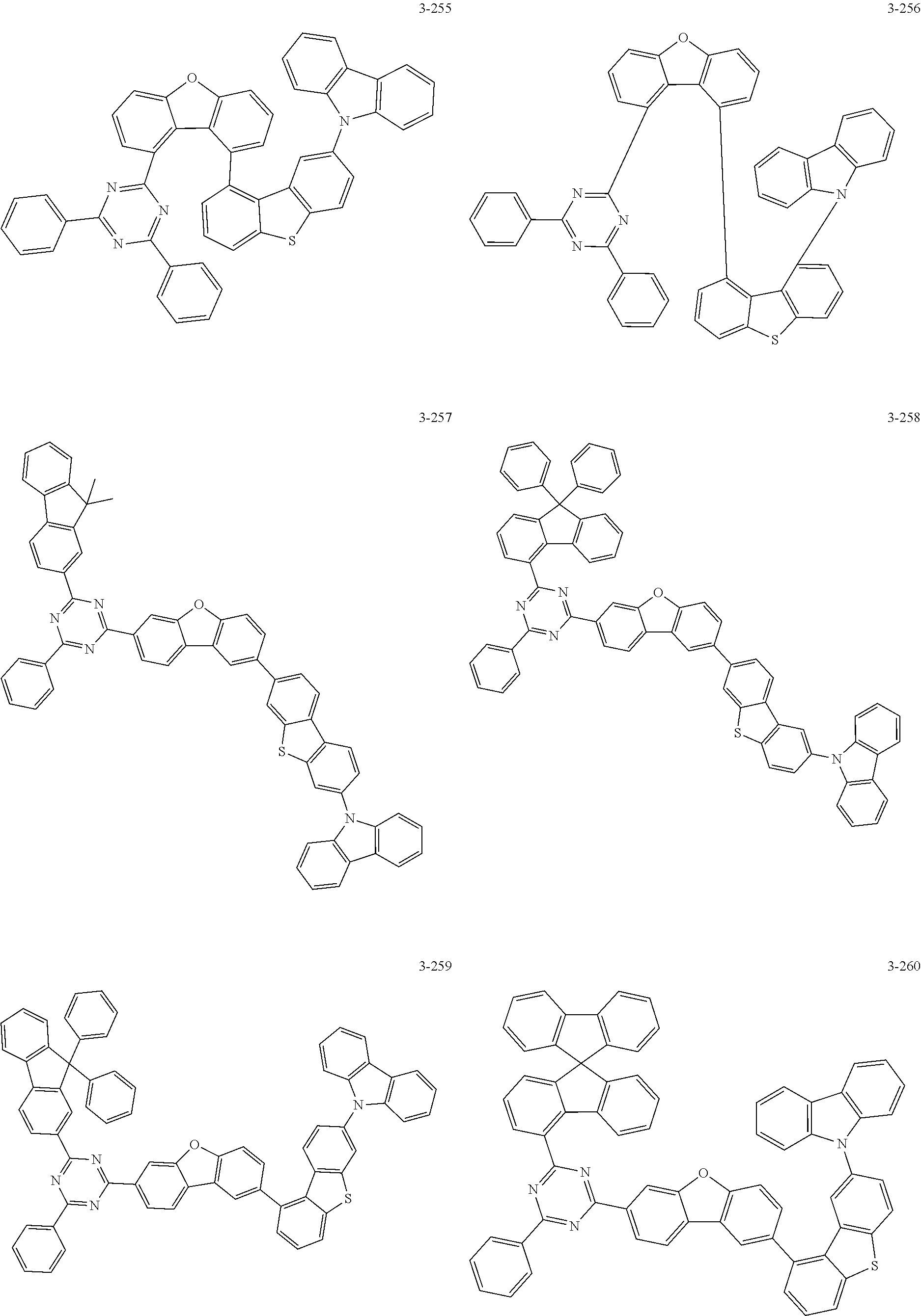

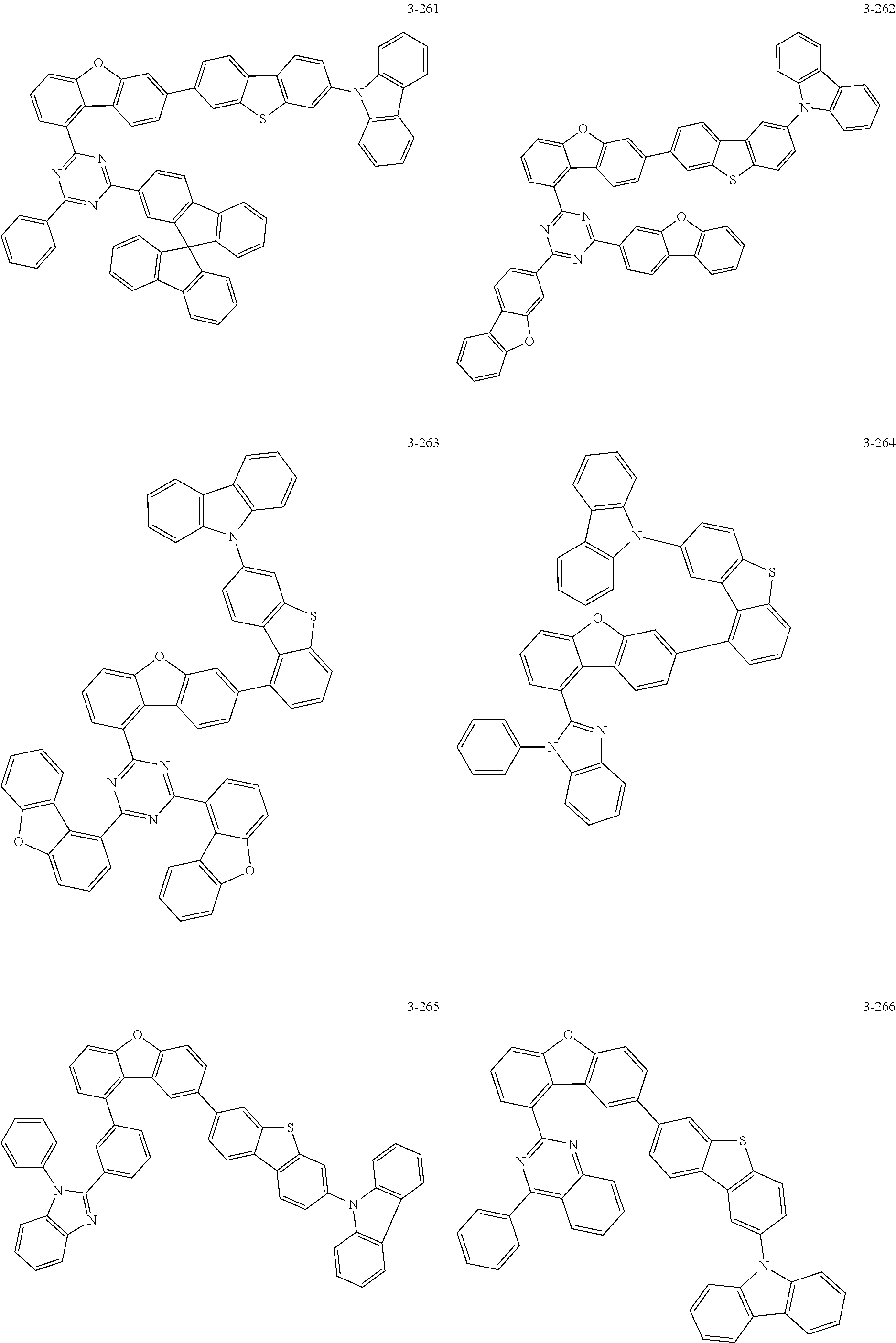

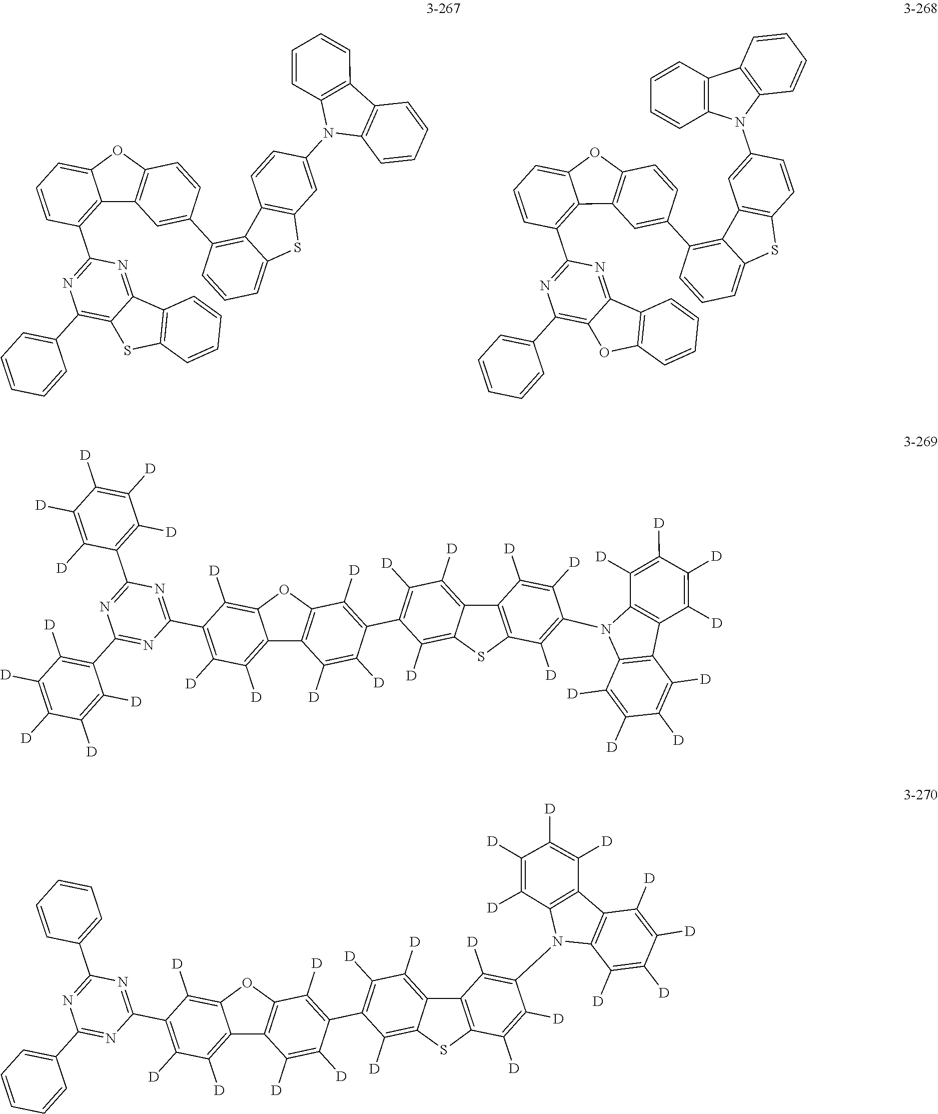

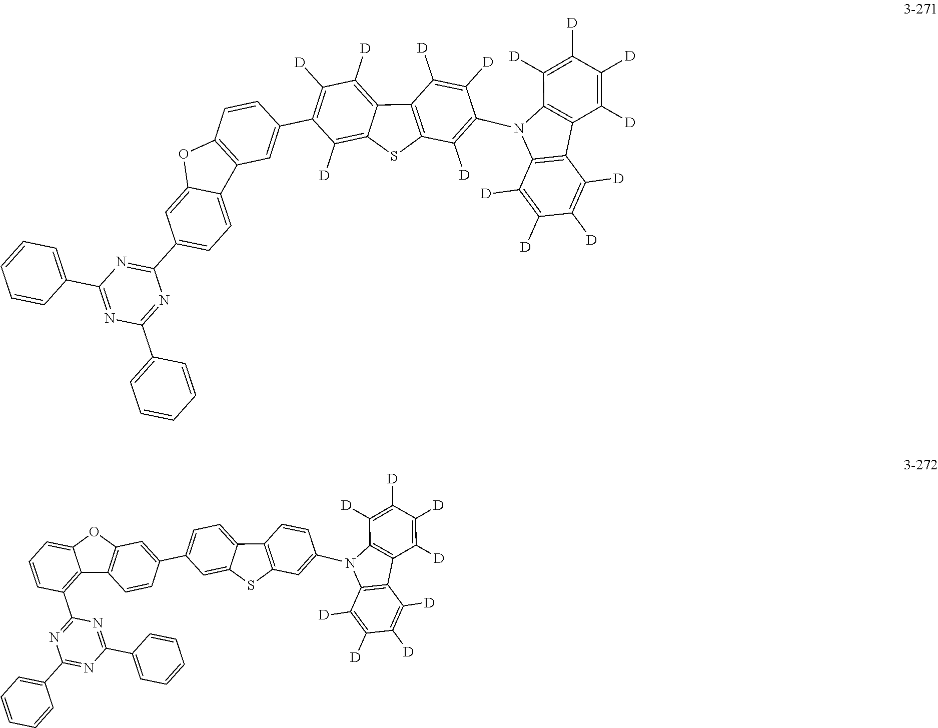 [SMILES: [2H]c1c(-c2ccc3oc4cc(-c5nc(-c6ccccc6)nc(-c6ccccc6)n5)ccc4c3c2)c([2H])c2sc3c([2H])c(-n4c5c([2H])c([2H])c([2H])c([2H])c5c5c([2H])c([2H])c([2H])c([2H])c54)c([2H])c([2H])c3c2c1[2H] ; [2H]c1c([2H])c([2H])c2c(c1[2H])c1c([2H])c([2H])c([2H])c([2H])c1n2-c1ccc2c(c1)sc1cc(-c3ccc4c(c3)oc3cccc(-c5nc(-c6ccccc6)nc(-c6ccccc6)n5)c34)ccc12]

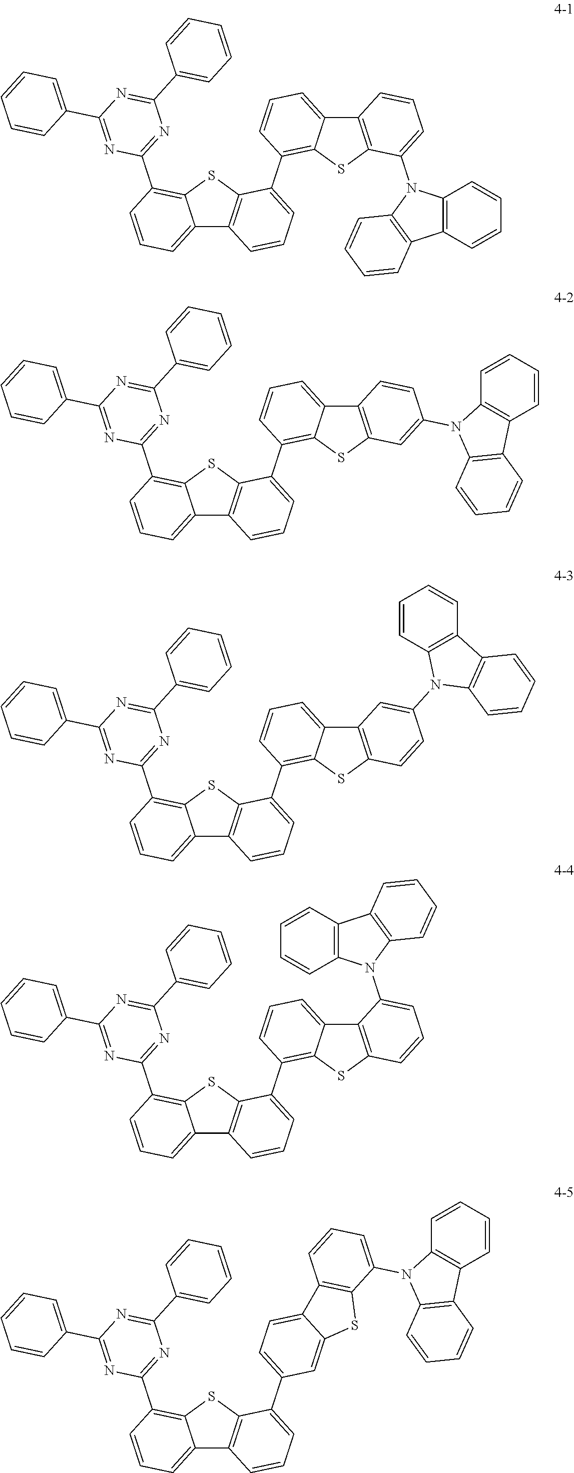 [SMILES: c1ccc(-c2nc(-c3ccccc3)nc(-c3cccc4c3sc3c(-c5cccc6c5sc5c(-n7c8ccccc8c8ccccc87)cccc56)cccc34)n2)cc1 ; c1ccc(-c2nc(-c3ccccc3)nc(-c3cccc4c3sc3c(-c5cccc6c5sc5cc(-n7c8ccccc8c8ccccc87)ccc56)cccc34)n2)cc1 ; c1ccc(-c2nc(-c3ccccc3)nc(-c3cccc4c3sc3c(-c5cccc6c5sc5ccc(-n7c8ccccc8c8ccccc87)cc56)cccc34)n2)cc1 ; c1ccc(-c2nc(-c3ccccc3)nc(-c3cccc4c3sc3c(-c5cccc6c5sc5cccc(-n7c8ccccc8c8ccccc87)c56)cccc34)n2)cc1 ; c1ccc(-c2nc(-c3ccccc3)nc(-c3cccc4c3sc3c(-c5ccc6c(c5)sc5c(-n7c8ccccc8c8ccccc87)cccc56)cccc34)n2)cc1]

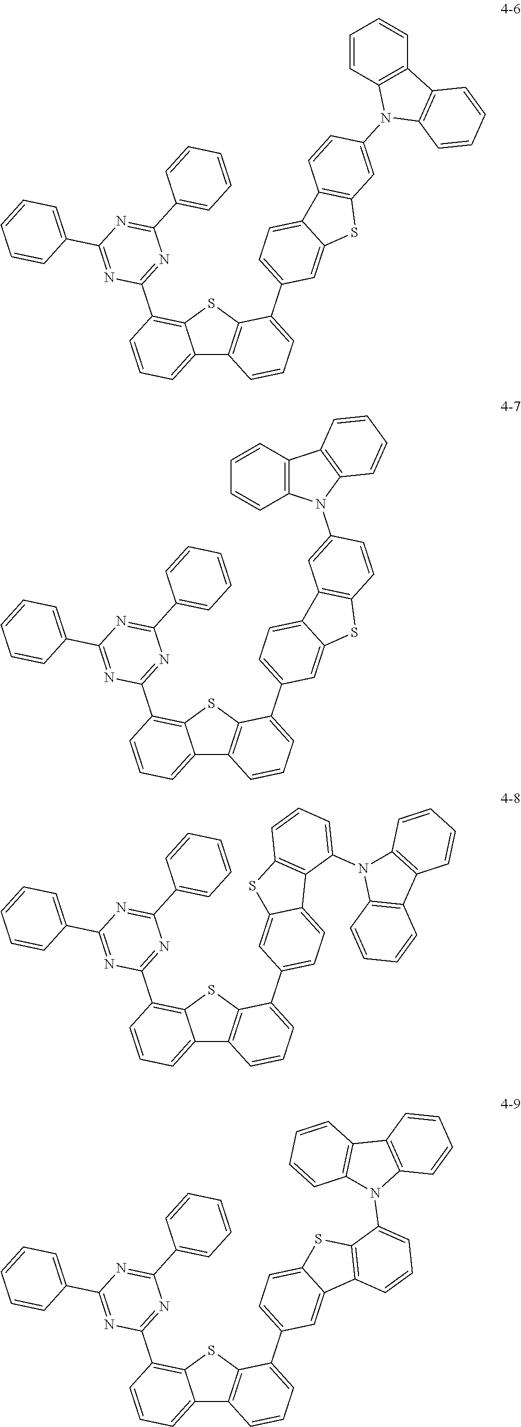

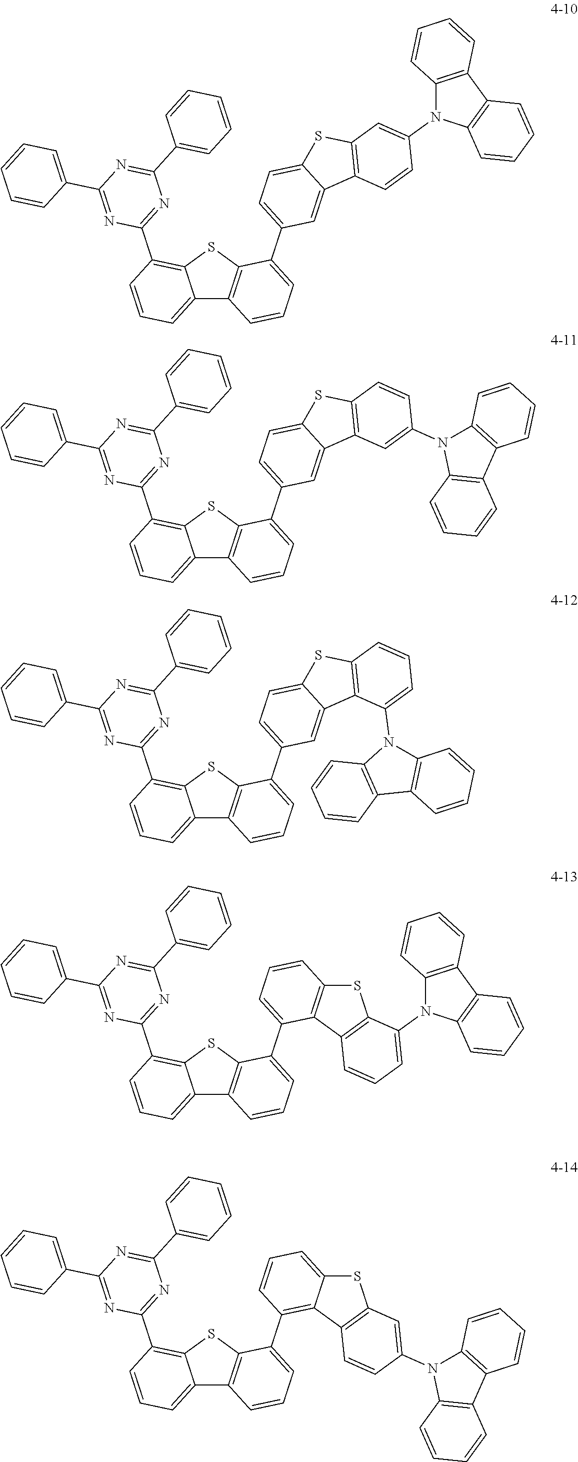

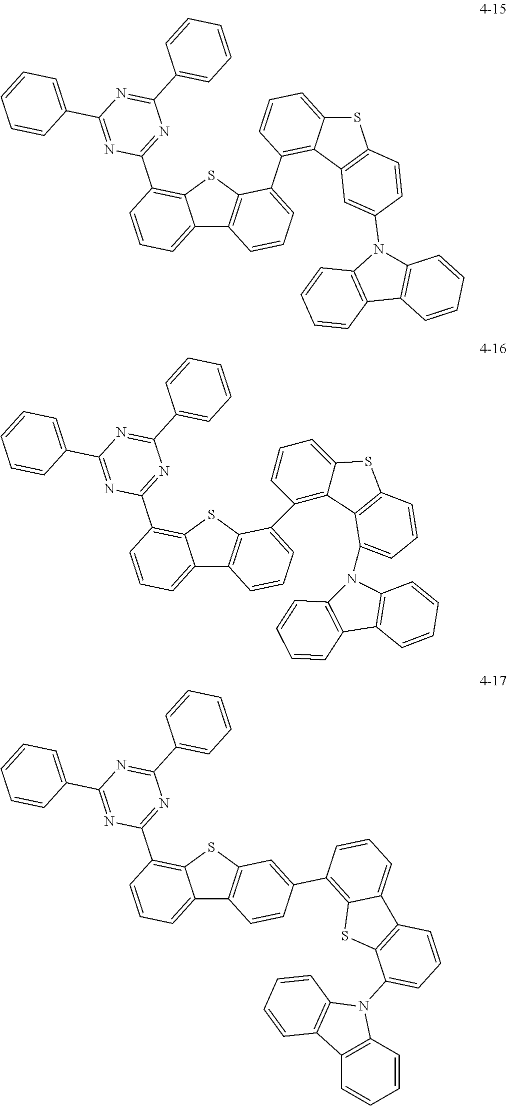 [SMILES: c1ccc(-c2nc(-c3ccccc3)nc(-c3cccc4c3sc3c(-c5cccc6sc7ccc(-n8c9ccccc9c9ccccc98)cc7c56)cccc34)n2)cc1 ; c1ccc(-c2nc(-c3ccccc3)nc(-c3cccc4c3sc3c(-c5cccc6sc7cccc(-n8c9ccccc9c9ccccc98)c7c56)cccc34)n2)cc1 ; c1ccc(-c2nc(-c3ccccc3)nc(-c3cccc4c3sc3cc(-c5cccc6c5sc5c(-n7c8ccccc8c8ccccc87)cccc56)ccc34)n2)cc1]

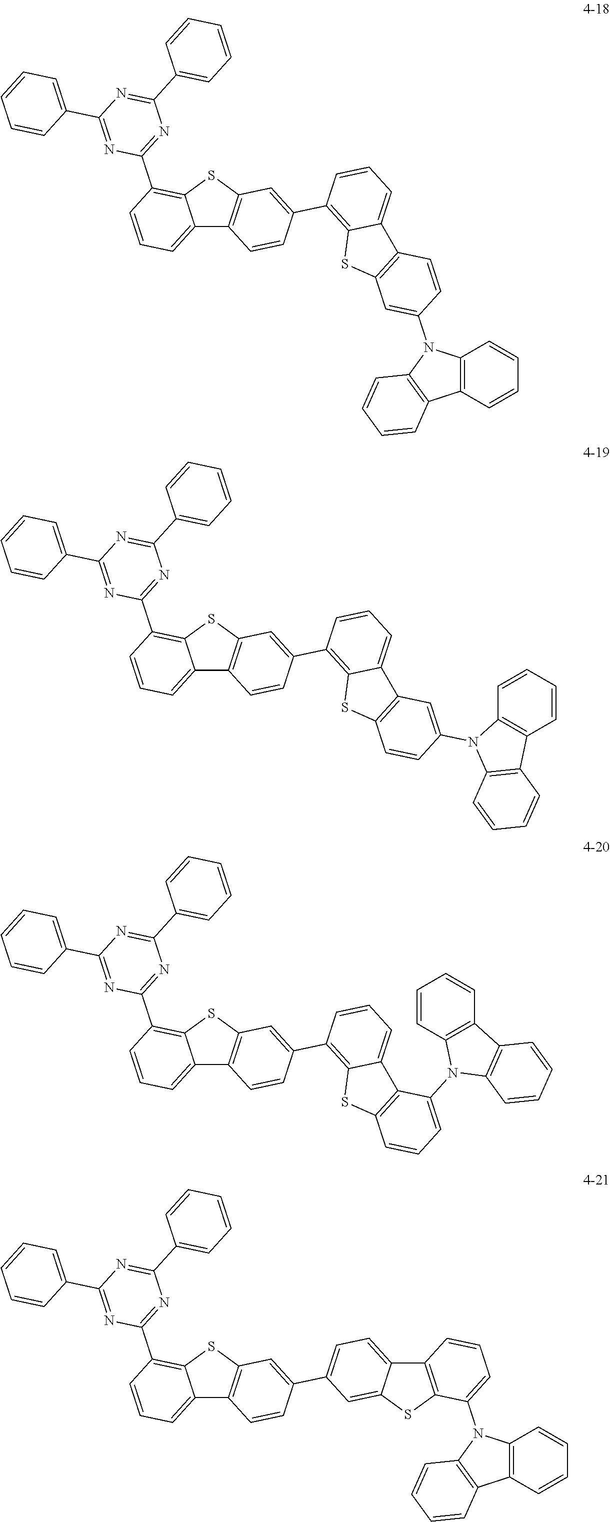 [SMILES: c1ccc(-c2nc(-c3ccccc3)nc(-c3cccc4c3sc3cc(-c5cccc6c5sc5cc(-n7c8ccccc8c8ccccc87)ccc56)ccc34)n2)cc1 ; c1ccc(-c2nc(-c3ccccc3)nc(-c3cccc4c3sc3cc(-c5cccc6c5sc5ccc(-n7c8ccccc8c8ccccc87)cc56)ccc34)n2)cc1 ; c1ccc(-c2nc(-c3ccccc3)nc(-c3cccc4c3sc3cc(-c5cccc6c5sc5cccc(-n7c8ccccc8c8ccccc87)c56)ccc34)n2)cc1 ; c1ccc(-c2nc(-c3ccccc3)nc(-c3cccc4c3sc3cc(-c5ccc6c(c5)sc5c(-n7c8ccccc8c8ccccc87)cccc56)ccc34)n2)cc1]

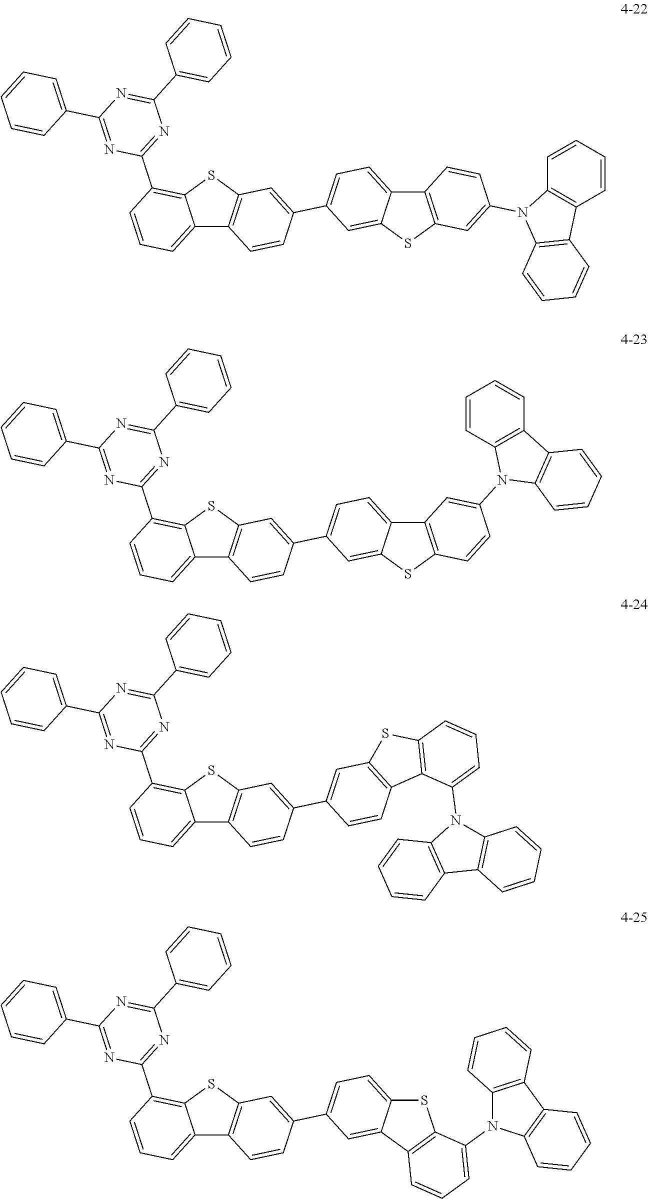

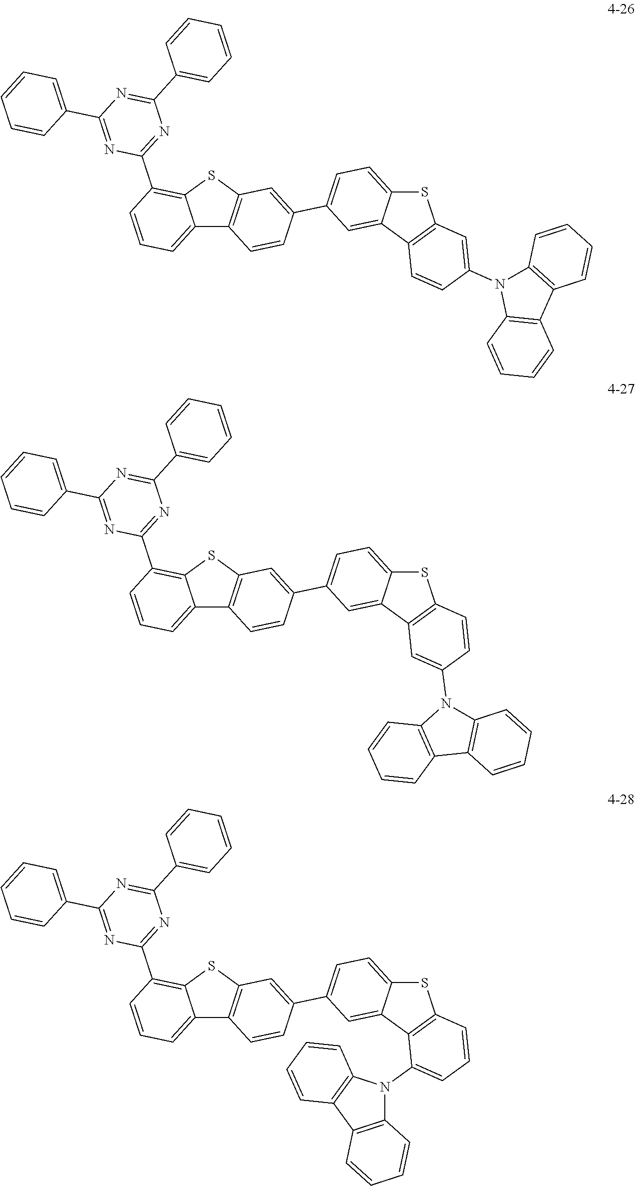 [SMILES: c1ccc(-c2nc(-c3ccccc3)nc(-c3cccc4c3sc3cc(-c5ccc6sc7cc(-n8c9ccccc9c9ccccc98)ccc7c6c5)ccc34)n2)cc1 ; c1ccc(-c2nc(-c3ccccc3)nc(-c3cccc4c3sc3cc(-c5ccc6sc7ccc(-n8c9ccccc9c9ccccc98)cc7c6c5)ccc34)n2)cc1 ; c1ccc(-c2nc(-c3ccccc3)nc(-c3cccc4c3sc3cc(-c5ccc6sc7cccc(-n8c9ccccc9c9ccccc98)c7c6c5)ccc34)n2)cc1]

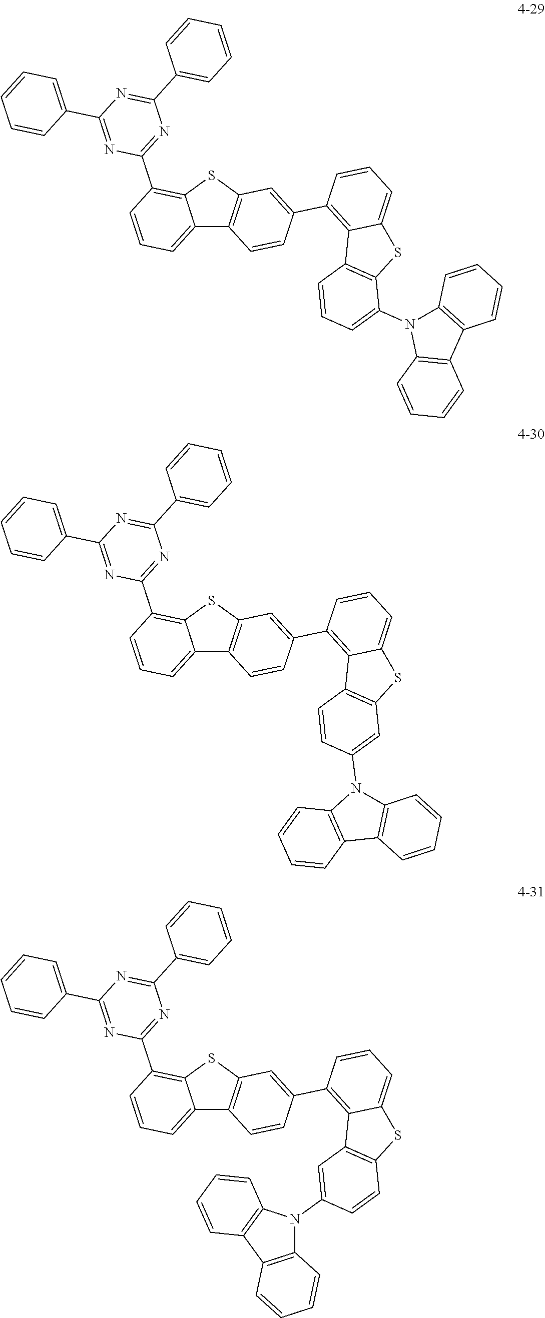 [SMILES: c1ccc(-c2nc(-c3ccccc3)nc(-c3cccc4c3sc3cc(-c5cccc6sc7c(-n8c9ccccc9c9ccccc98)cccc7c56)ccc34)n2)cc1 ; c1ccc(-c2nc(-c3ccccc3)nc(-c3cccc4c3sc3cc(-c5cccc6sc7cc(-n8c9ccccc9c9ccccc98)ccc7c56)ccc34)n2)cc1 ; c1ccc(-c2nc(-c3ccccc3)nc(-c3cccc4c3sc3cc(-c5cccc6sc7ccc(-n8c9ccccc9c9ccccc98)cc7c56)ccc34)n2)cc1]

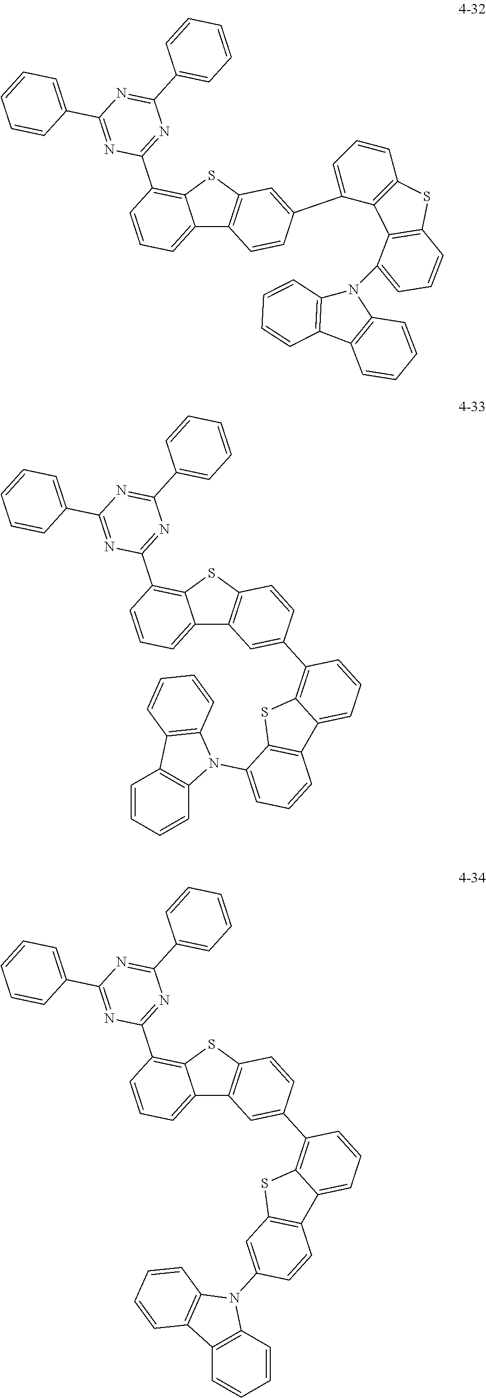 [SMILES: c1ccc(-c2nc(-c3ccccc3)nc(-c3cccc4c3sc3cc(-c5cccc6sc7cccc(-n8c9ccccc9c9ccccc98)c7c56)ccc34)n2)cc1 ; c1ccc(-c2nc(-c3ccccc3)nc(-c3cccc4c3sc3ccc(-c5cccc6c5sc5c(-n7c8ccccc8c8ccccc87)cccc56)cc34)n2)cc1 ; c1ccc(-c2nc(-c3ccccc3)nc(-c3cccc4c3sc3ccc(-c5cccc6c5sc5cc(-n7c8ccccc8c8ccccc87)ccc56)cc34)n2)cc1]

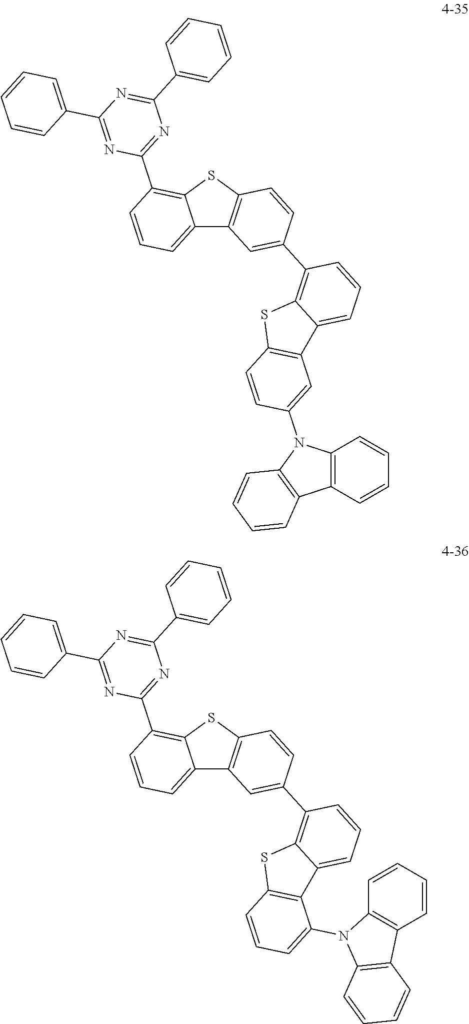 [SMILES: c1ccc(-c2nc(-c3ccccc3)nc(-c3cccc4c3sc3ccc(-c5cccc6c5sc5ccc(-n7c8ccccc8c8ccccc87)cc56)cc34)n2)cc1 ; c1ccc(-c2nc(-c3ccccc3)nc(-c3cccc4c3sc3ccc(-c5cccc6c5sc5cccc(-n7c8ccccc8c8ccccc87)c56)cc34)n2)cc1]

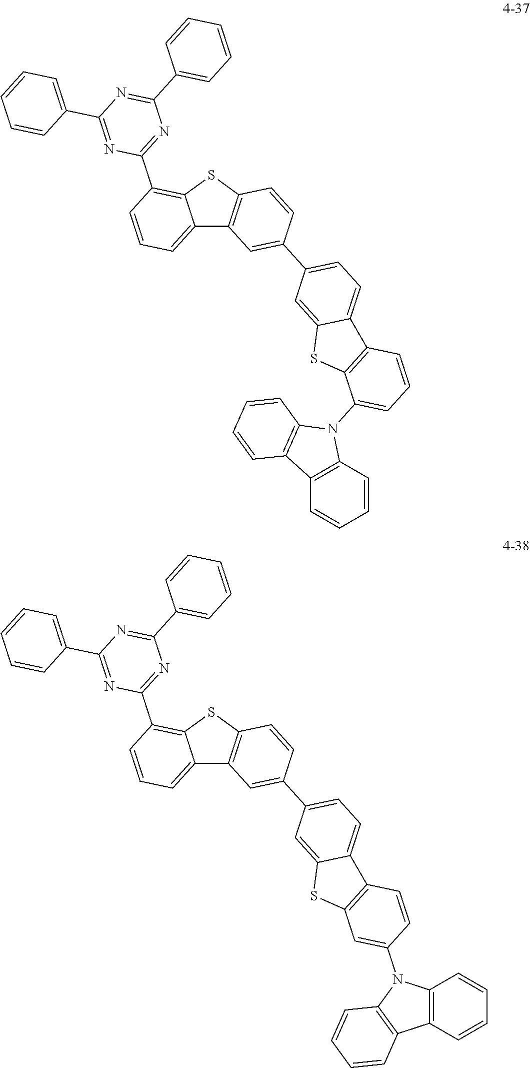 [SMILES: c1ccc(-c2nc(-c3ccccc3)nc(-c3cccc4c3sc3ccc(-c5ccc6c(c5)sc5c(-n7c8ccccc8c8ccccc87)cccc56)cc34)n2)cc1 ; c1ccc(-c2nc(-c3ccccc3)nc(-c3cccc4c3sc3ccc(-c5ccc6c(c5)sc5cc(-n7c8ccccc8c8ccccc87)ccc56)cc34)n2)cc1]

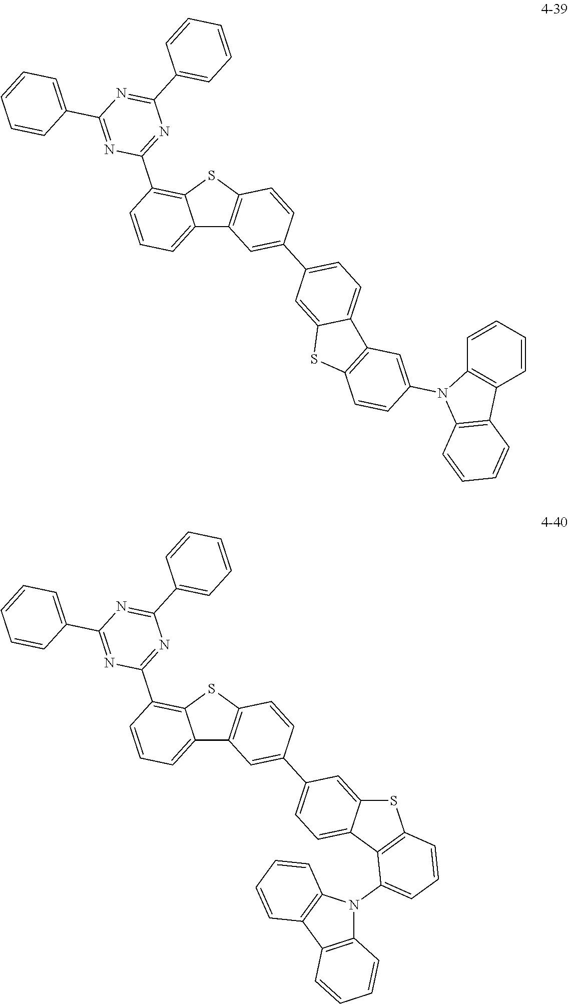 [SMILES: c1ccc(-c2nc(-c3ccccc3)nc(-c3cccc4c3sc3ccc(-c5ccc6c(c5)sc5ccc(-n7c8ccccc8c8ccccc87)cc56)cc34)n2)cc1 ; c1ccc(-c2nc(-c3ccccc3)nc(-c3cccc4c3sc3ccc(-c5ccc6c(c5)sc5cccc(-n7c8ccccc8c8ccccc87)c56)cc34)n2)cc1]

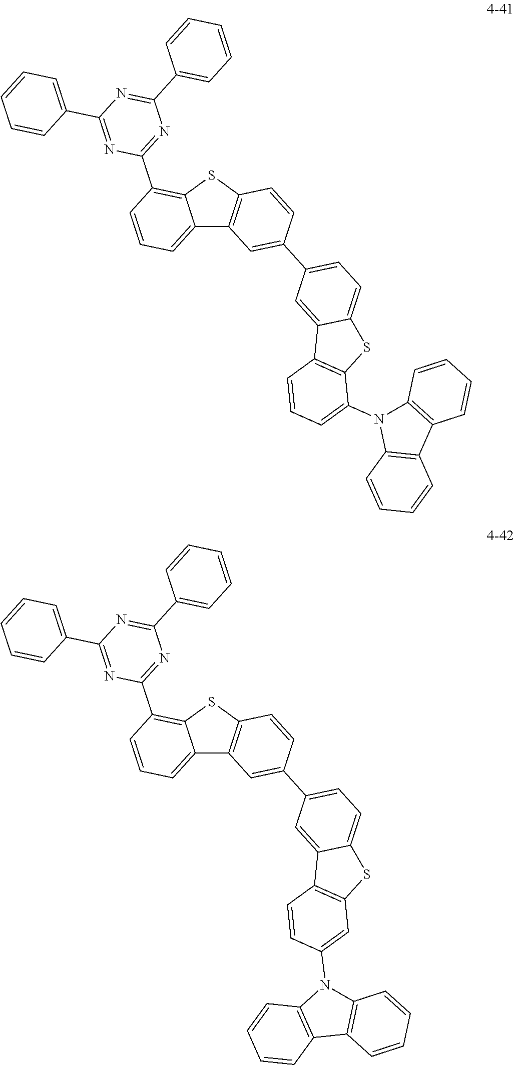 [SMILES: c1ccc(-c2nc(-c3ccccc3)nc(-c3cccc4c3sc3ccc(-c5ccc6sc7c(-n8c9ccccc9c9ccccc98)cccc7c6c5)cc34)n2)cc1 ; c1ccc(-c2nc(-c3ccccc3)nc(-c3cccc4c3sc3ccc(-c5ccc6sc7cc(-n8c9ccccc9c9ccccc98)ccc7c6c5)cc34)n2)cc1]

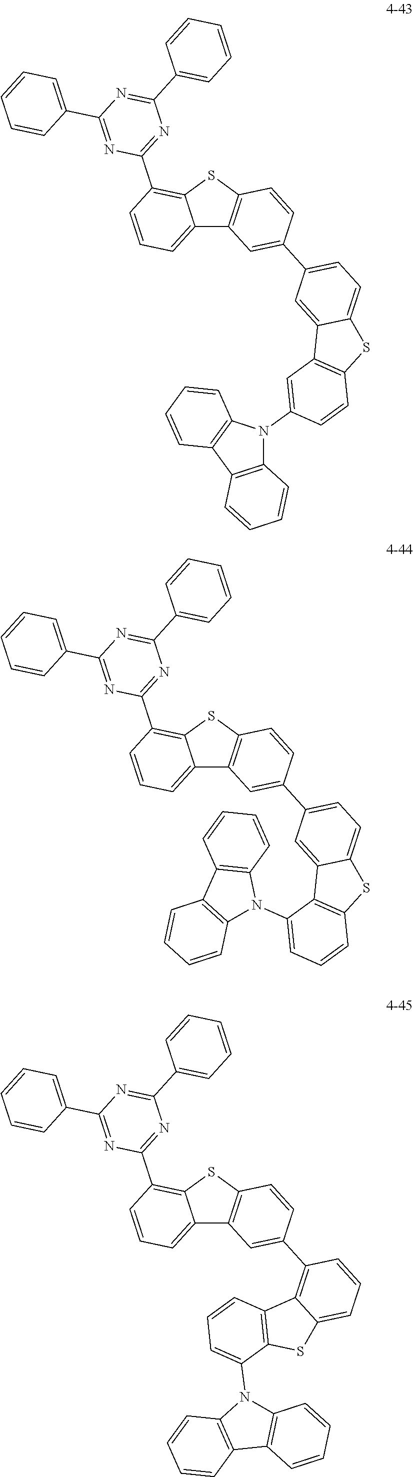

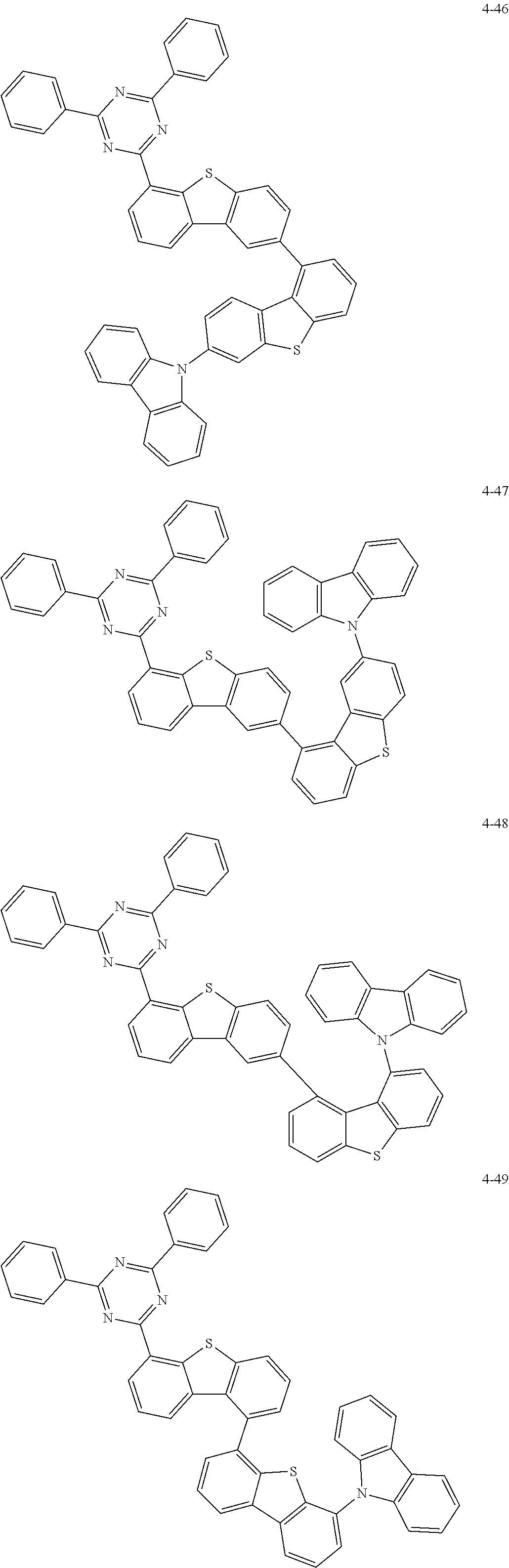

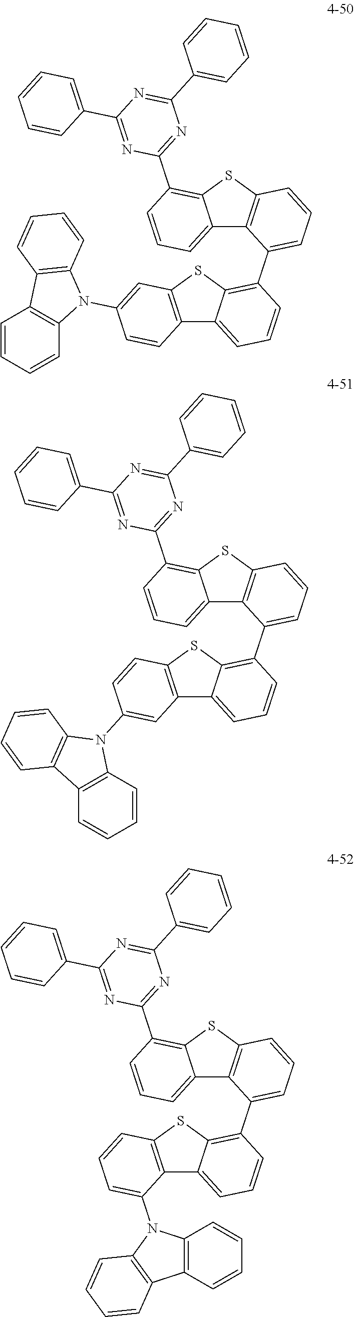 [SMILES: c1ccc(-c2nc(-c3ccccc3)nc(-c3cccc4c3sc3cccc(-c5cccc6c5sc5cc(-n7c8ccccc8c8ccccc87)ccc56)c34)n2)cc1 ; c1ccc(-c2nc(-c3ccccc3)nc(-c3cccc4c3sc3cccc(-c5cccc6c5sc5ccc(-n7c8ccccc8c8ccccc87)cc56)c34)n2)cc1 ; c1ccc(-c2nc(-c3ccccc3)nc(-c3cccc4c3sc3cccc(-c5cccc6c5sc5cccc(-n7c8ccccc8c8ccccc87)c56)c34)n2)cc1]

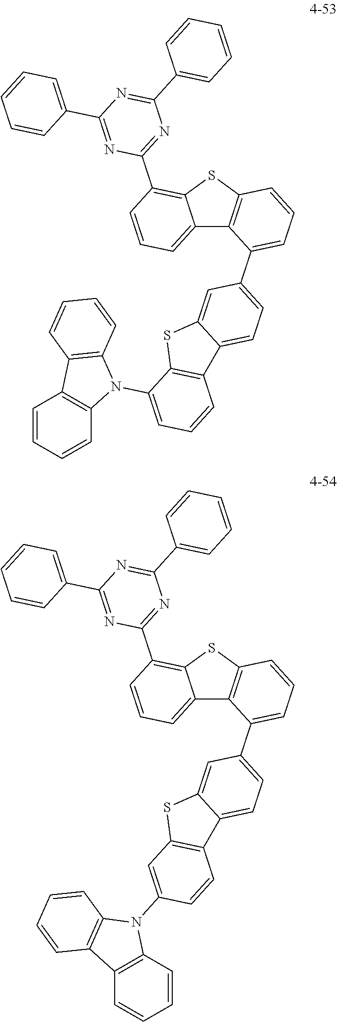 [SMILES: c1ccc(-c2nc(-c3ccccc3)nc(-c3cccc4c3sc3cccc(-c5ccc6c(c5)sc5c(-n7c8ccccc8c8ccccc87)cccc56)c34)n2)cc1 ; c1ccc(-c2nc(-c3ccccc3)nc(-c3cccc4c3sc3cccc(-c5ccc6c(c5)sc5cc(-n7c8ccccc8c8ccccc87)ccc56)c34)n2)cc1]

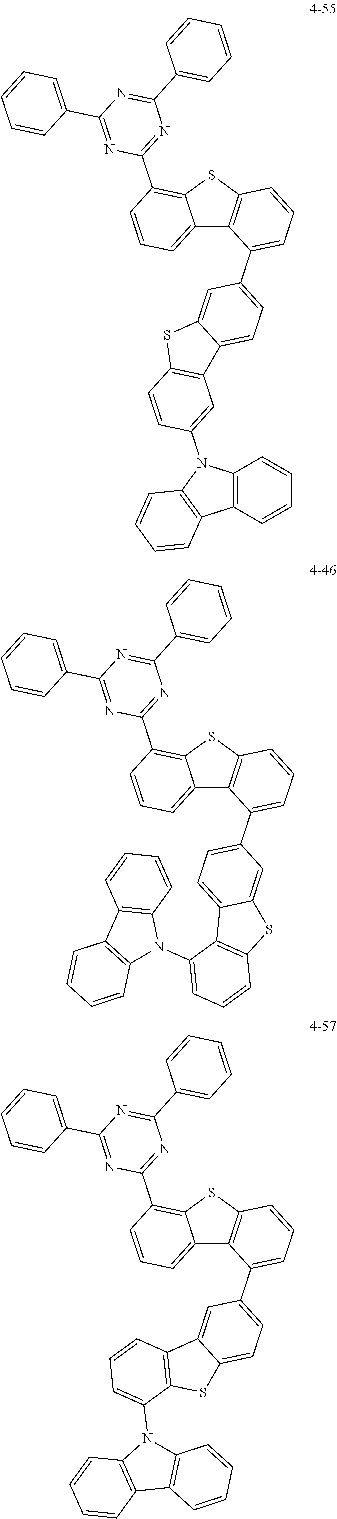

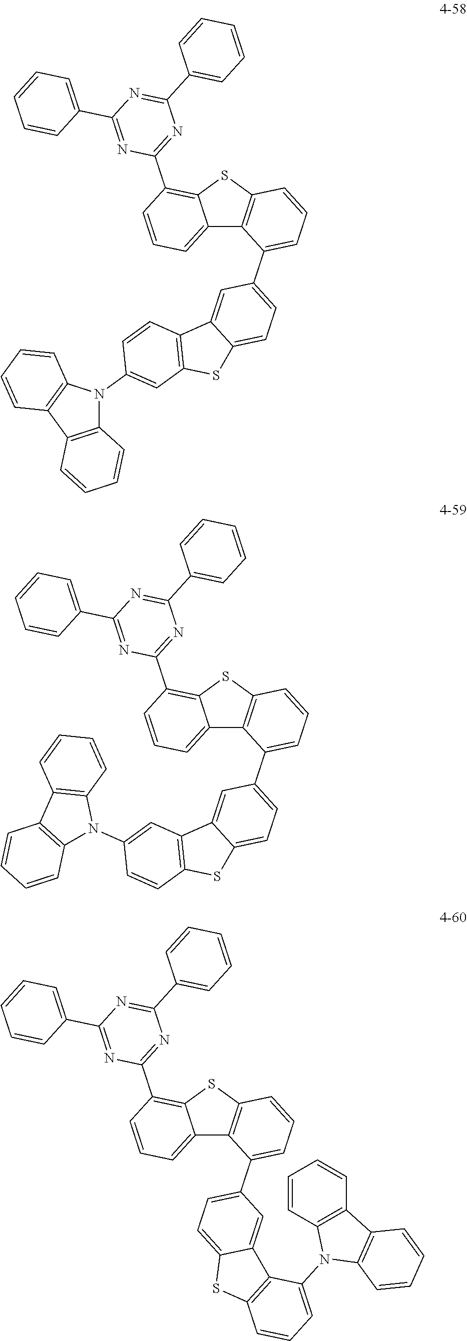 [SMILES: c1ccc(-c2nc(-c3ccccc3)nc(-c3cccc4c3sc3cccc(-c5ccc6sc7cc(-n8c9ccccc9c9ccccc98)ccc7c6c5)c34)n2)cc1 ; c1ccc(-c2nc(-c3ccccc3)nc(-c3cccc4c3sc3cccc(-c5ccc6sc7ccc(-n8c9ccccc9c9ccccc98)cc7c6c5)c34)n2)cc1 ; c1ccc(-c2nc(-c3ccccc3)nc(-c3cccc4c3sc3cccc(-c5ccc6sc7cccc(-n8c9ccccc9c9ccccc98)c7c6c5)c34)n2)cc1]

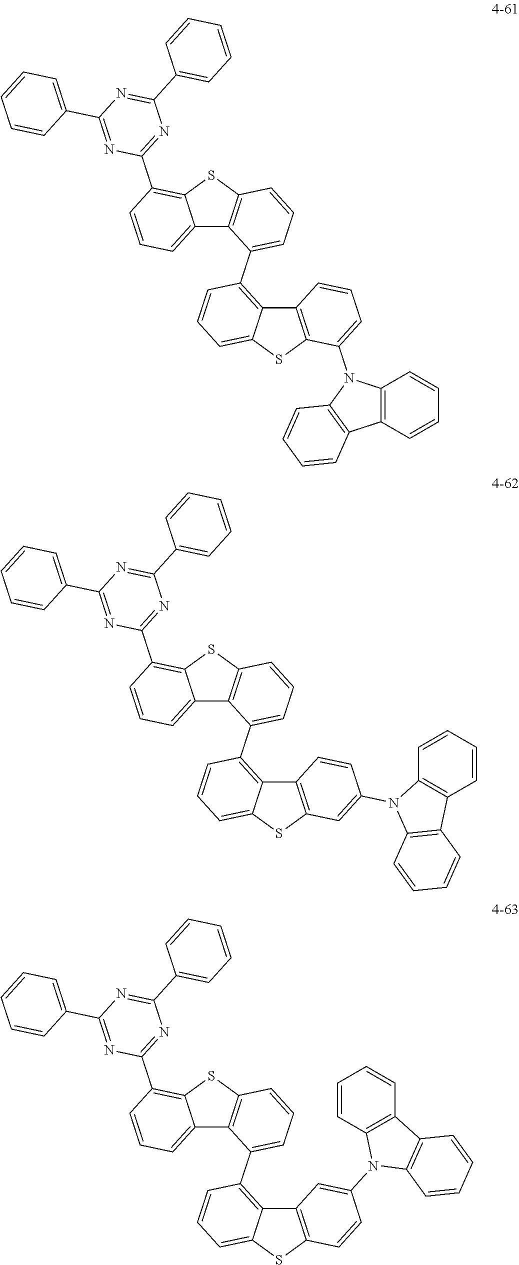 [SMILES: c1ccc(-c2nc(-c3ccccc3)nc(-c3cccc4c3sc3cccc(-c5cccc6sc7c(-n8c9ccccc9c9ccccc98)cccc7c56)c34)n2)cc1 ; c1ccc(-c2nc(-c3ccccc3)nc(-c3cccc4c3sc3cccc(-c5cccc6sc7cc(-n8c9ccccc9c9ccccc98)ccc7c56)c34)n2)cc1 ; c1ccc(-c2nc(-c3ccccc3)nc(-c3cccc4c3sc3cccc(-c5cccc6sc7ccc(-n8c9ccccc9c9ccccc98)cc7c56)c34)n2)cc1]

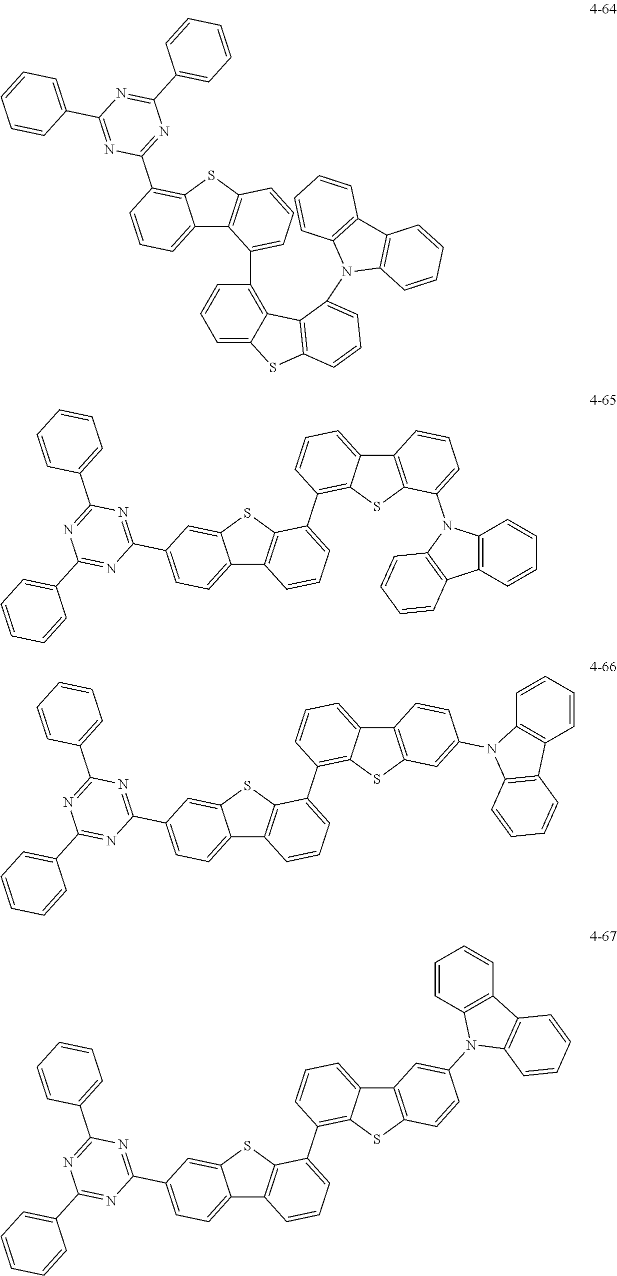

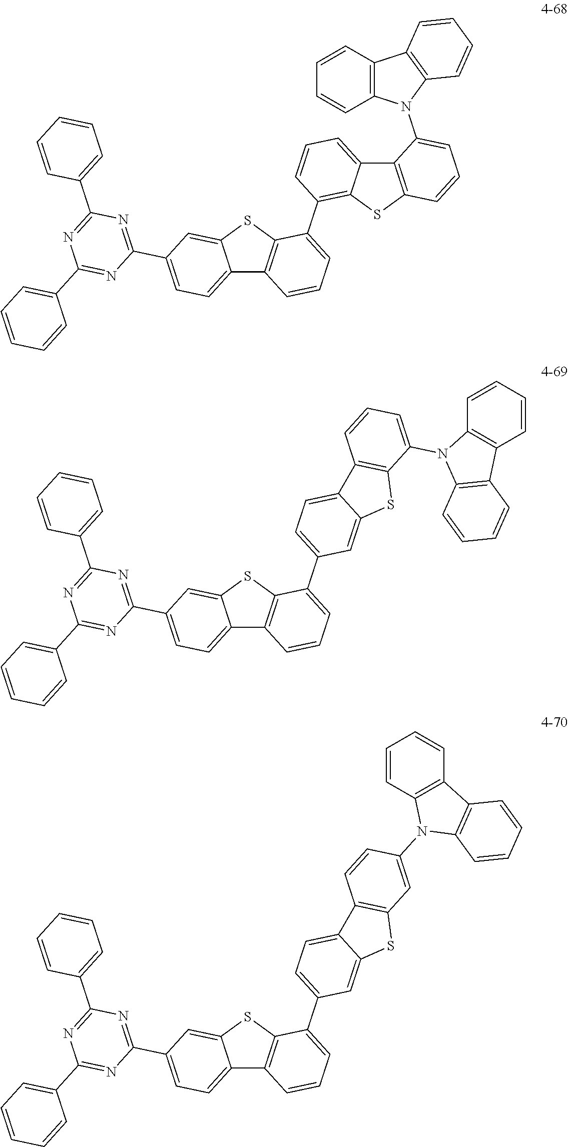 [SMILES: c1ccc(-c2nc(-c3ccccc3)nc(-c3ccc4c(c3)sc3c(-c5cccc6c5sc5cccc(-n7c8ccccc8c8ccccc87)c56)cccc34)n2)cc1 ; c1ccc(-c2nc(-c3ccccc3)nc(-c3ccc4c(c3)sc3c(-c5ccc6c(c5)sc5c(-n7c8ccccc8c8ccccc87)cccc56)cccc34)n2)cc1 ; c1ccc(-c2nc(-c3ccccc3)nc(-c3ccc4c(c3)sc3c(-c5ccc6c(c5)sc5cc(-n7c8ccccc8c8ccccc87)ccc56)cccc34)n2)cc1]

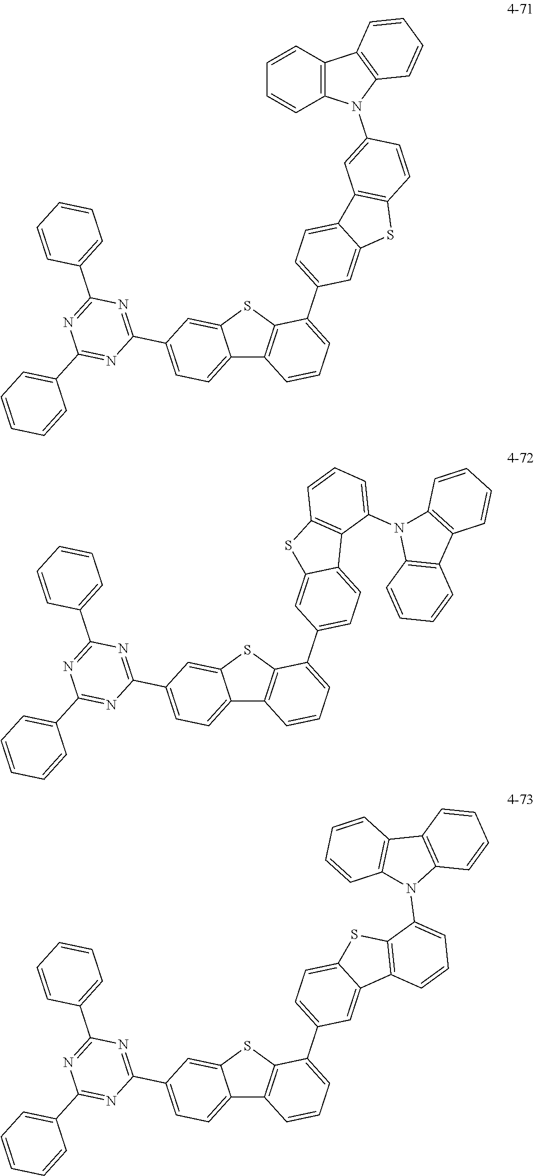 [SMILES: c1ccc(-c2nc(-c3ccccc3)nc(-c3ccc4c(c3)sc3c(-c5ccc6c(c5)sc5ccc(-n7c8ccccc8c8ccccc87)cc56)cccc34)n2)cc1 ; c1ccc(-c2nc(-c3ccccc3)nc(-c3ccc4c(c3)sc3c(-c5ccc6c(c5)sc5cccc(-n7c8ccccc8c8ccccc87)c56)cccc34)n2)cc1 ; c1ccc(-c2nc(-c3ccccc3)nc(-c3ccc4c(c3)sc3c(-c5ccc6sc7c(-n8c9ccccc9c9ccccc98)cccc7c6c5)cccc34)n2)cc1]

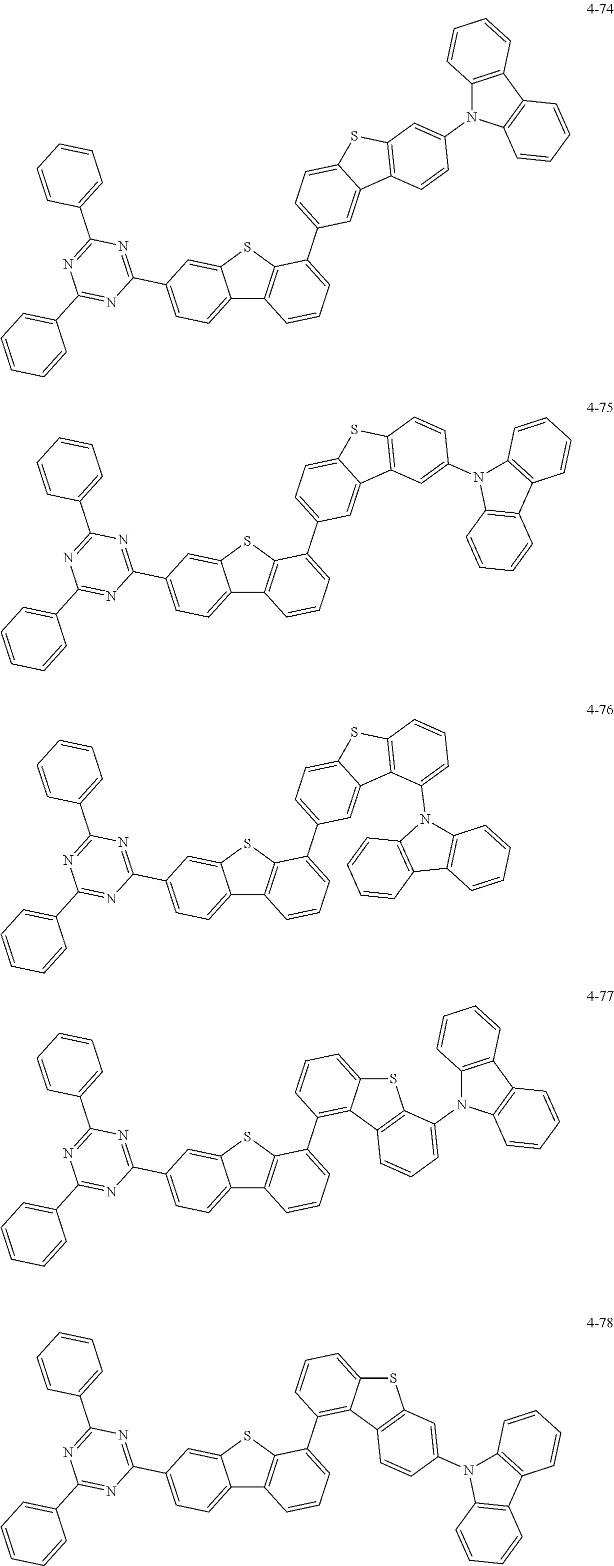

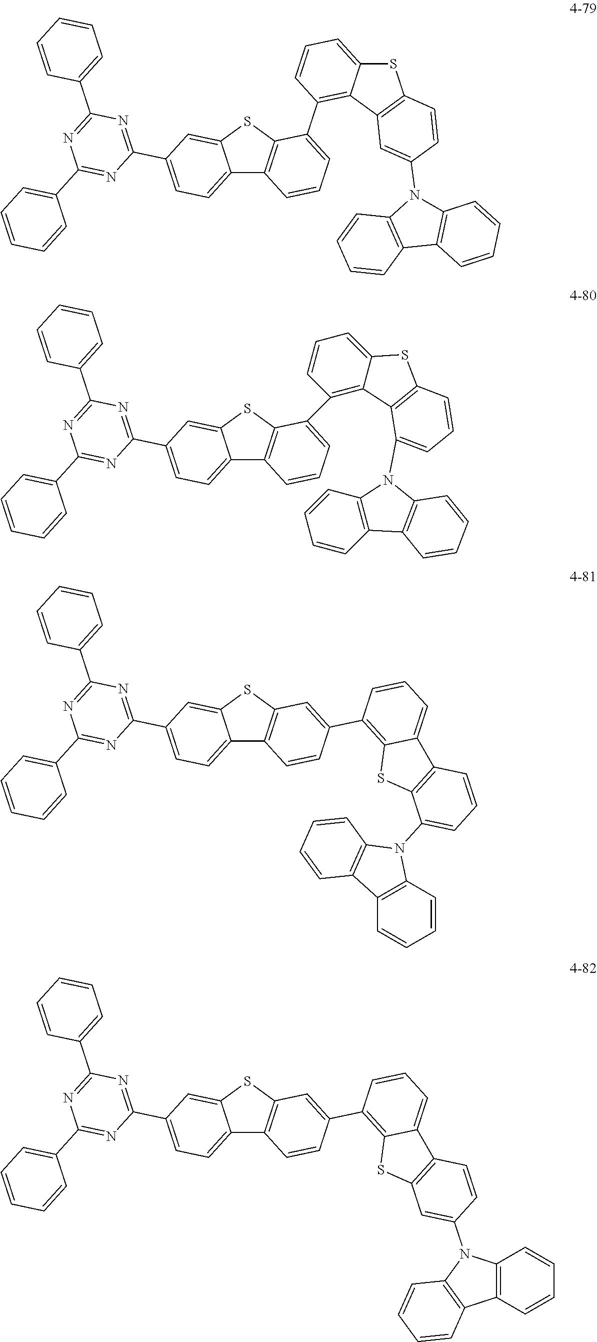 [SMILES: c1ccc(-c2nc(-c3ccccc3)nc(-c3ccc4c(c3)sc3c(-c5cccc6sc7ccc(-n8c9ccccc9c9ccccc98)cc7c56)cccc34)n2)cc1 ; c1ccc(-c2nc(-c3ccccc3)nc(-c3ccc4c(c3)sc3c(-c5cccc6sc7cccc(-n8c9ccccc9c9ccccc98)c7c56)cccc34)n2)cc1 ; c1ccc(-c2nc(-c3ccccc3)nc(-c3ccc4c(c3)sc3cc(-c5cccc6c5sc5c(-n7c8ccccc8c8ccccc87)cccc56)ccc34)n2)cc1 ; c1ccc(-c2nc(-c3ccccc3)nc(-c3ccc4c(c3)sc3cc(-c5cccc6c5sc5cc(-n7c8ccccc8c8ccccc87)ccc56)ccc34)n2)cc1]

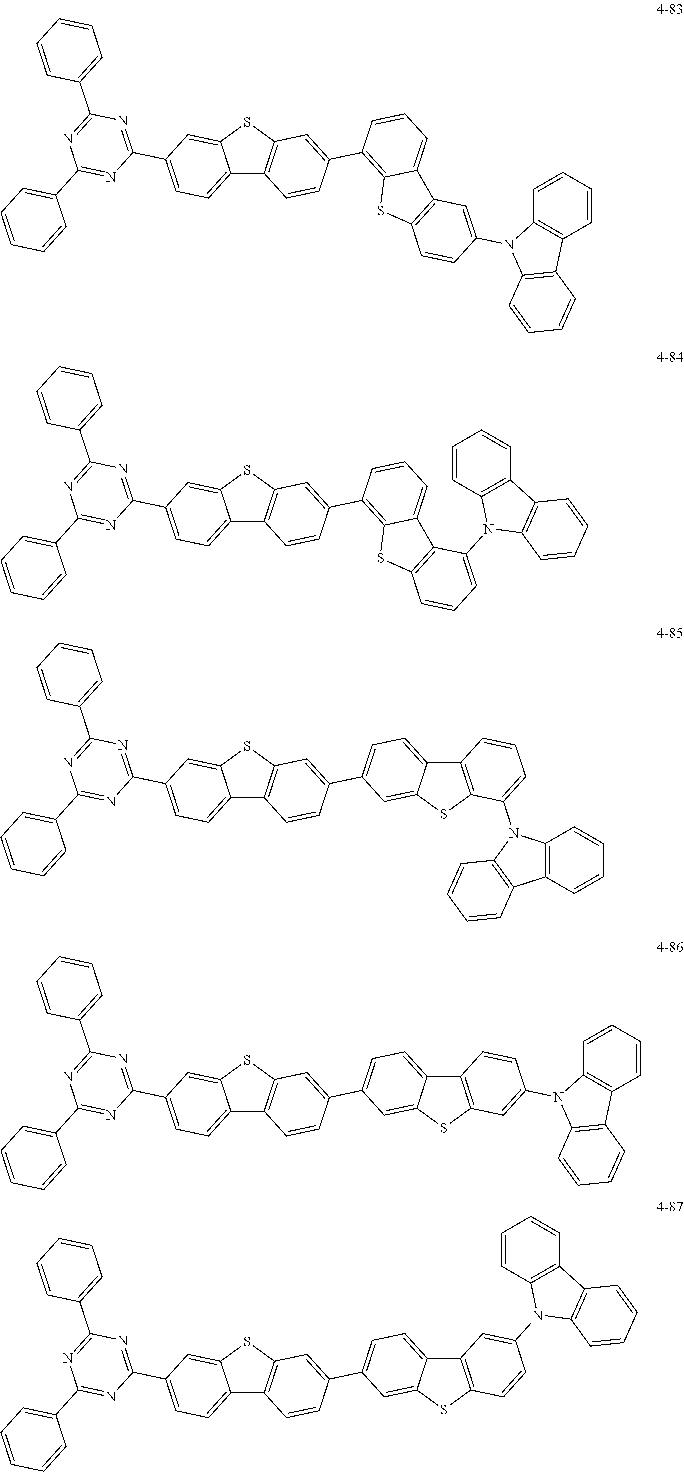

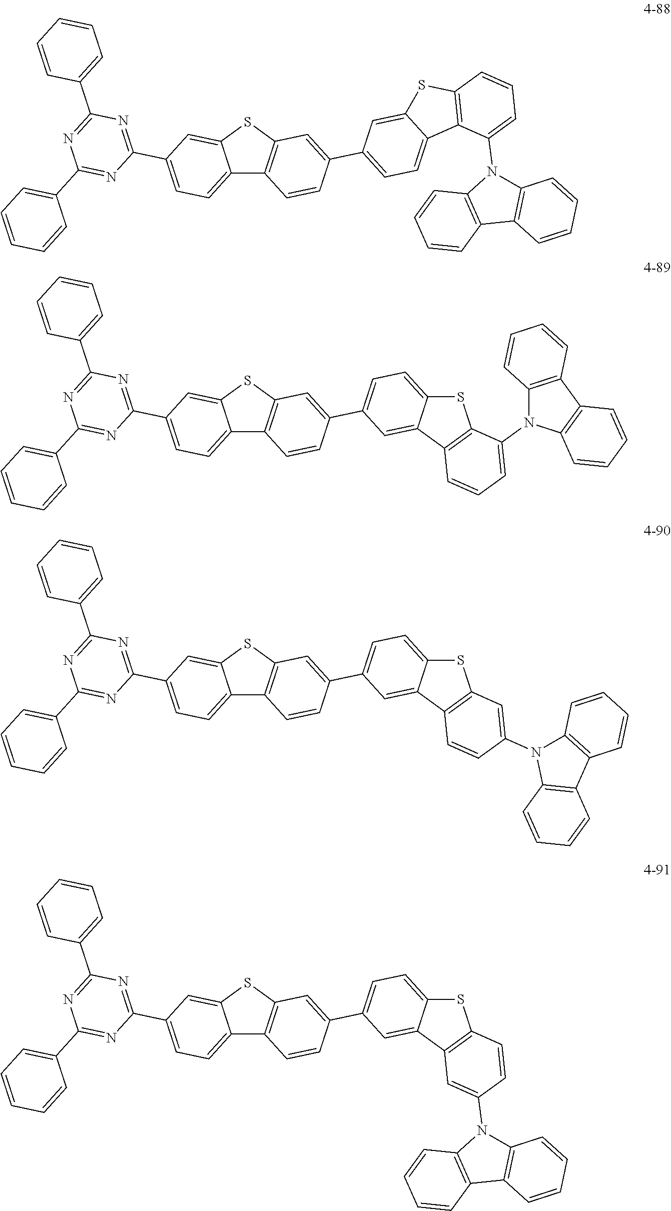

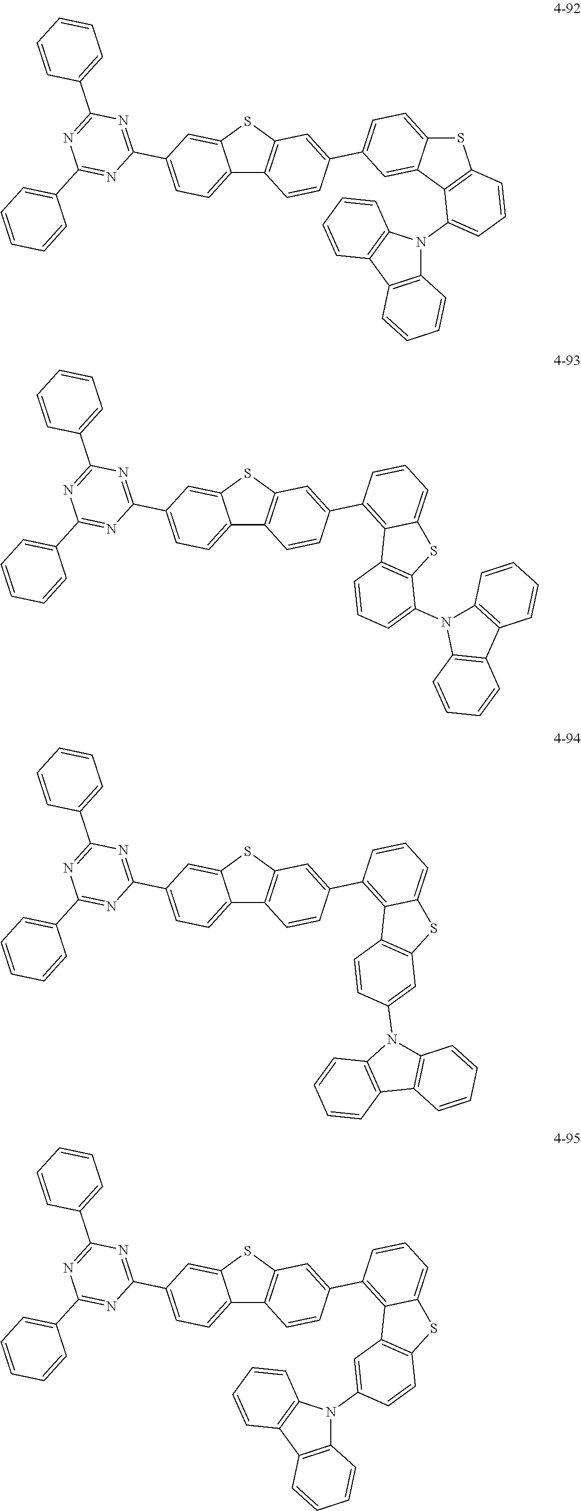 [SMILES: c1ccc(-c2nc(-c3ccccc3)nc(-c3ccc4c(c3)sc3cc(-c5ccc6sc7cccc(-n8c9ccccc9c9ccccc98)c7c6c5)ccc34)n2)cc1 ; c1ccc(-c2nc(-c3ccccc3)nc(-c3ccc4c(c3)sc3cc(-c5cccc6sc7c(-n8c9ccccc9c9ccccc98)cccc7c56)ccc34)n2)cc1 ; c1ccc(-c2nc(-c3ccccc3)nc(-c3ccc4c(c3)sc3cc(-c5cccc6sc7cc(-n8c9ccccc9c9ccccc98)ccc7c56)ccc34)n2)cc1 ; c1ccc(-c2nc(-c3ccccc3)nc(-c3ccc4c(c3)sc3cc(-c5cccc6sc7ccc(-n8c9ccccc9c9ccccc98)cc7c56)ccc34)n2)cc1]

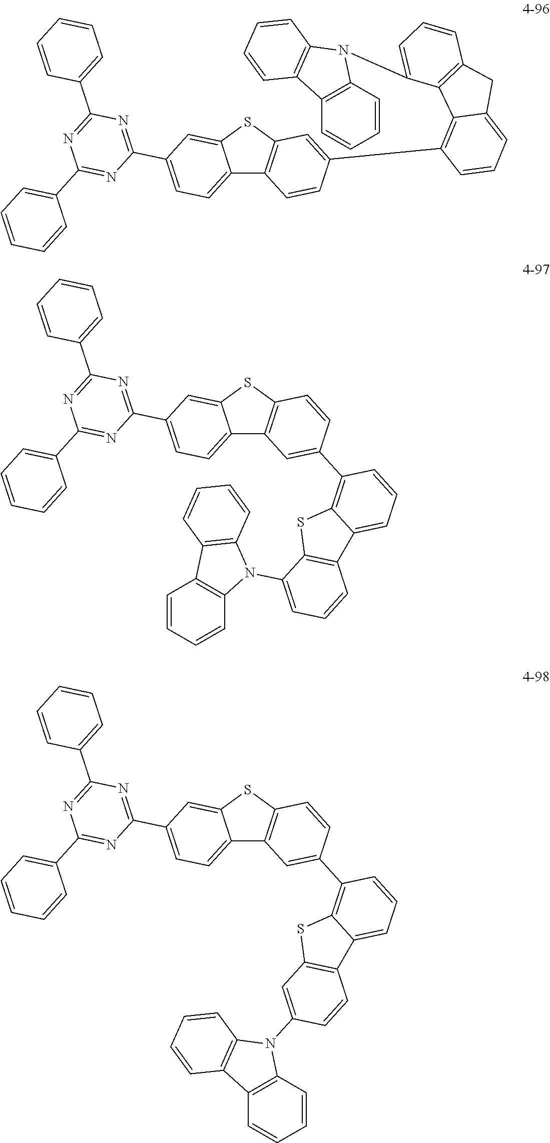

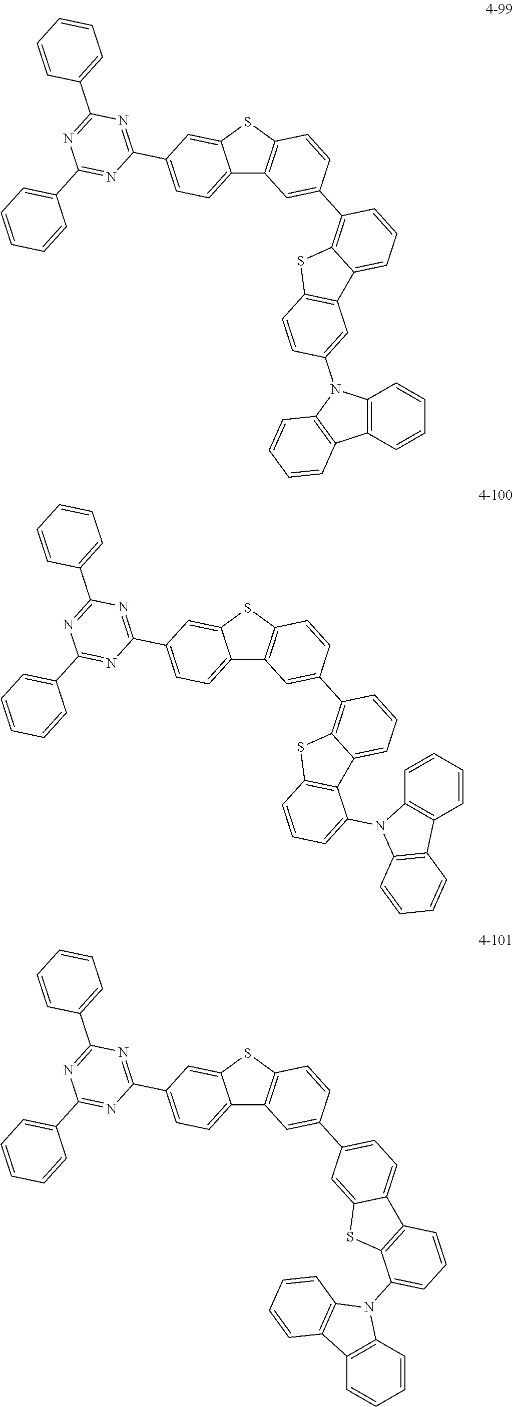 [SMILES: c1ccc(-c2nc(-c3ccccc3)nc(-c3ccc4c(c3)sc3ccc(-c5cccc6c5sc5ccc(-n7c8ccccc8c8ccccc87)cc56)cc34)n2)cc1 ; c1ccc(-c2nc(-c3ccccc3)nc(-c3ccc4c(c3)sc3ccc(-c5cccc6c5sc5cccc(-n7c8ccccc8c8ccccc87)c56)cc34)n2)cc1 ; c1ccc(-c2nc(-c3ccccc3)nc(-c3ccc4c(c3)sc3ccc(-c5ccc6c(c5)sc5c(-n7c8ccccc8c8ccccc87)cccc56)cc34)n2)cc1]

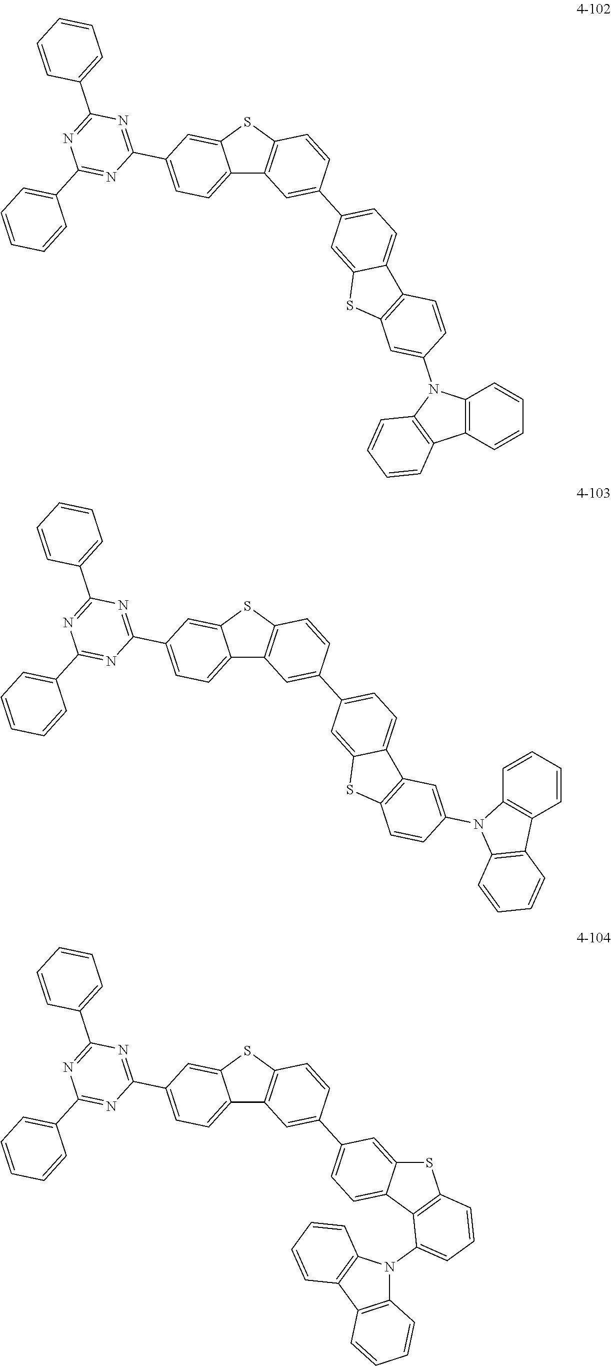 [SMILES: c1ccc(-c2nc(-c3ccccc3)nc(-c3ccc4c(c3)sc3ccc(-c5ccc6c(c5)sc5cc(-n7c8ccccc8c8ccccc87)ccc56)cc34)n2)cc1 ; c1ccc(-c2nc(-c3ccccc3)nc(-c3ccc4c(c3)sc3ccc(-c5ccc6c(c5)sc5ccc(-n7c8ccccc8c8ccccc87)cc56)cc34)n2)cc1 ; c1ccc(-c2nc(-c3ccccc3)nc(-c3ccc4c(c3)sc3ccc(-c5ccc6c(c5)sc5cccc(-n7c8ccccc8c8ccccc87)c56)cc34)n2)cc1]

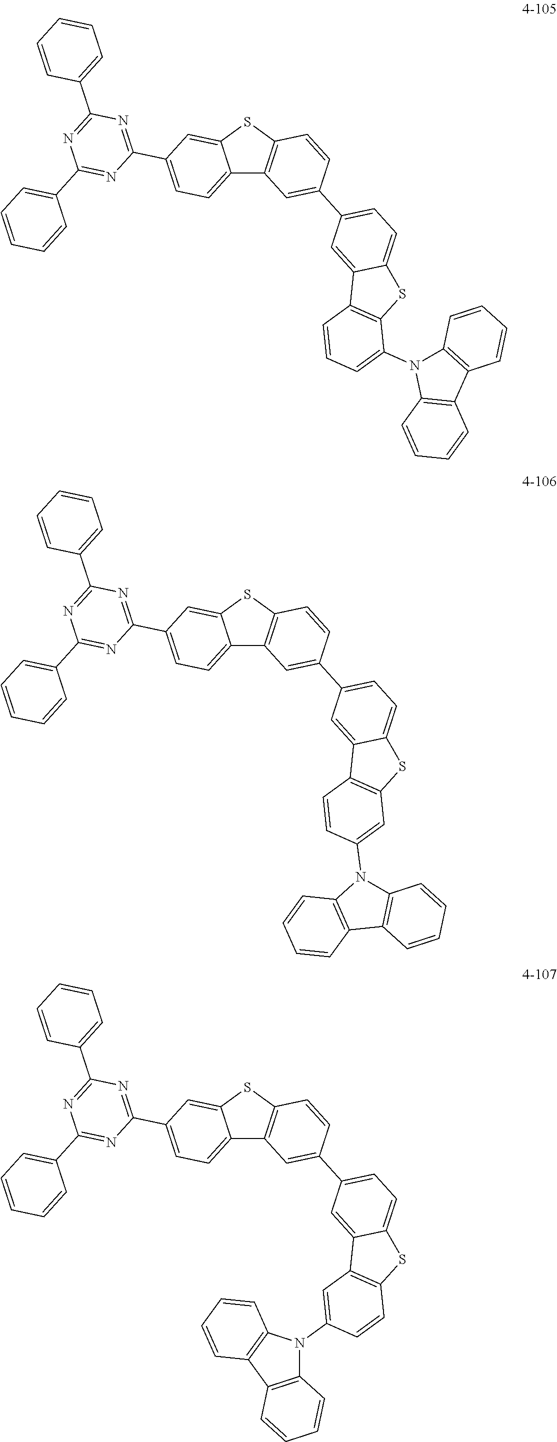 [SMILES: c1ccc(-c2nc(-c3ccccc3)nc(-c3ccc4c(c3)sc3ccc(-c5ccc6sc7c(-n8c9ccccc9c9ccccc98)cccc7c6c5)cc34)n2)cc1 ; c1ccc(-c2nc(-c3ccccc3)nc(-c3ccc4c(c3)sc3ccc(-c5ccc6sc7cc(-n8c9ccccc9c9ccccc98)ccc7c6c5)cc34)n2)cc1 ; c1ccc(-c2nc(-c3ccccc3)nc(-c3ccc4c(c3)sc3ccc(-c5ccc6sc7ccc(-n8c9ccccc9c9ccccc98)cc7c6c5)cc34)n2)cc1]

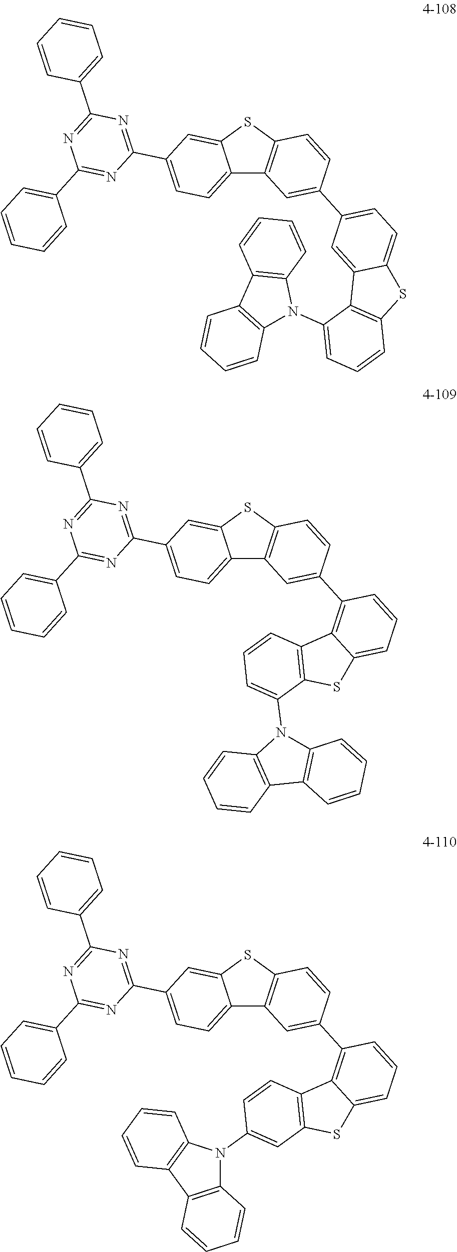 [SMILES: c1ccc(-c2nc(-c3ccccc3)nc(-c3ccc4c(c3)sc3ccc(-c5ccc6sc7cccc(-n8c9ccccc9c9ccccc98)c7c6c5)cc34)n2)cc1 ; c1ccc(-c2nc(-c3ccccc3)nc(-c3ccc4c(c3)sc3ccc(-c5cccc6sc7c(-n8c9ccccc9c9ccccc98)cccc7c56)cc34)n2)cc1 ; c1ccc(-c2nc(-c3ccccc3)nc(-c3ccc4c(c3)sc3ccc(-c5cccc6sc7cc(-n8c9ccccc9c9ccccc98)ccc7c56)cc34)n2)cc1]

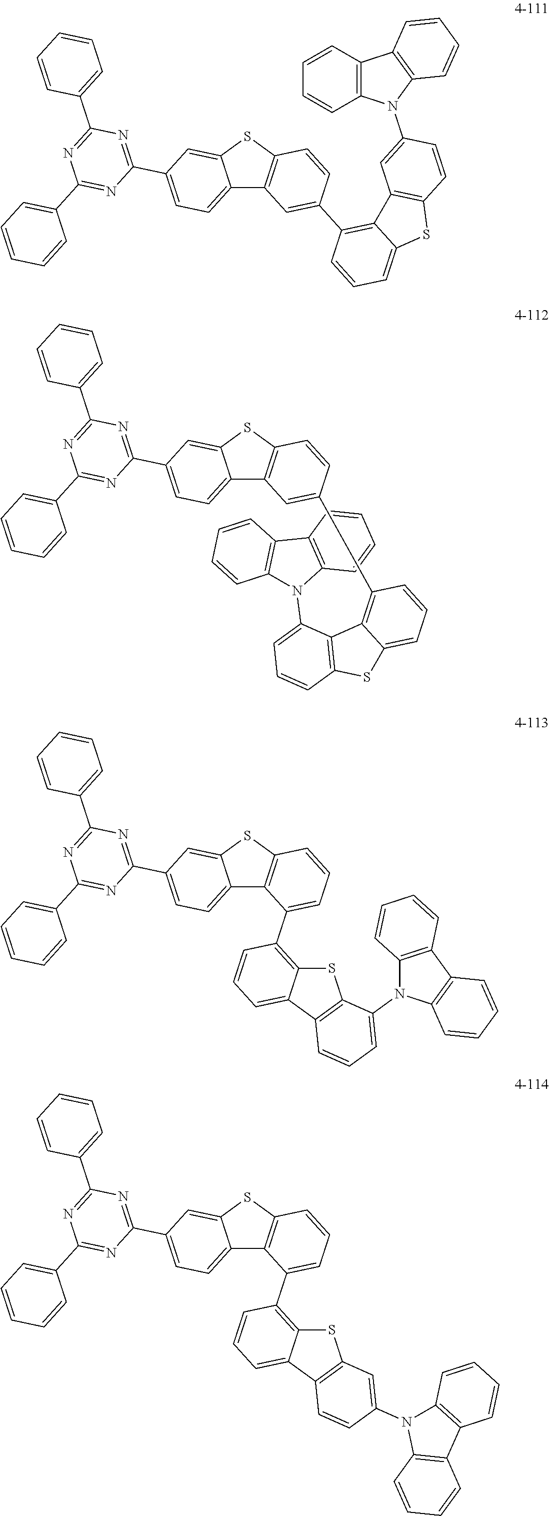 [SMILES: c1ccc(-c2nc(-c3ccccc3)nc(-c3ccc4c(c3)sc3ccc(-c5cccc6sc7ccc(-n8c9ccccc9c9ccccc98)cc7c56)cc34)n2)cc1 ; c1ccc(-c2nc(-c3ccccc3)nc(-c3ccc4c(c3)sc3ccc(-c5cccc6sc7cccc(-n8c9ccccc9c9ccccc98)c7c56)cc34)n2)cc1 ; c1ccc(-c2nc(-c3ccccc3)nc(-c3ccc4c(c3)sc3cccc(-c5cccc6c5sc5c(-n7c8ccccc8c8ccccc87)cccc56)c34)n2)cc1 ; c1ccc(-c2nc(-c3ccccc3)nc(-c3ccc4c(c3)sc3cccc(-c5cccc6c5sc5cc(-n7c8ccccc8c8ccccc87)ccc56)c34)n2)cc1]

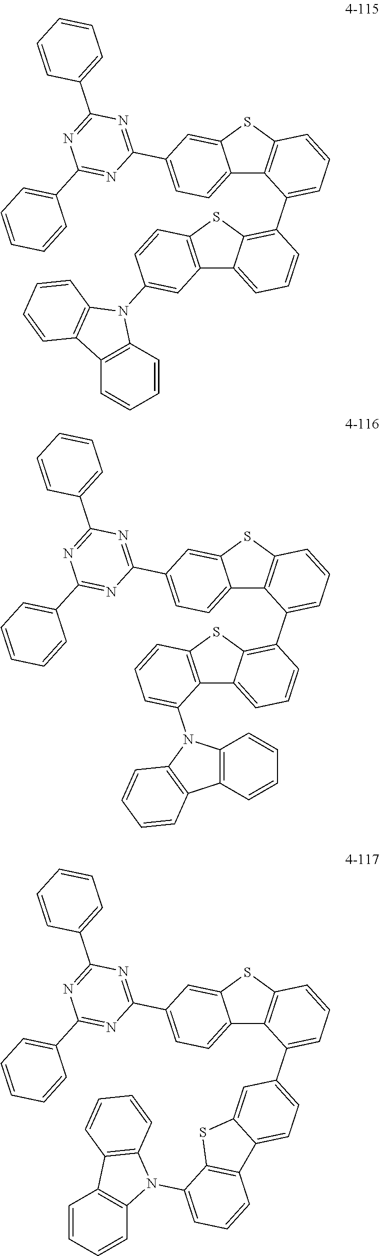 [SMILES: c1ccc(-c2nc(-c3ccccc3)nc(-c3ccc4c(c3)sc3cccc(-c5cccc6c5sc5ccc(-n7c8ccccc8c8ccccc87)cc56)c34)n2)cc1 ; c1ccc(-c2nc(-c3ccccc3)nc(-c3ccc4c(c3)sc3cccc(-c5cccc6c5sc5cccc(-n7c8ccccc8c8ccccc87)c56)c34)n2)cc1 ; c1ccc(-c2nc(-c3ccccc3)nc(-c3ccc4c(c3)sc3cccc(-c5ccc6c(c5)sc5c(-n7c8ccccc8c8ccccc87)cccc56)c34)n2)cc1]

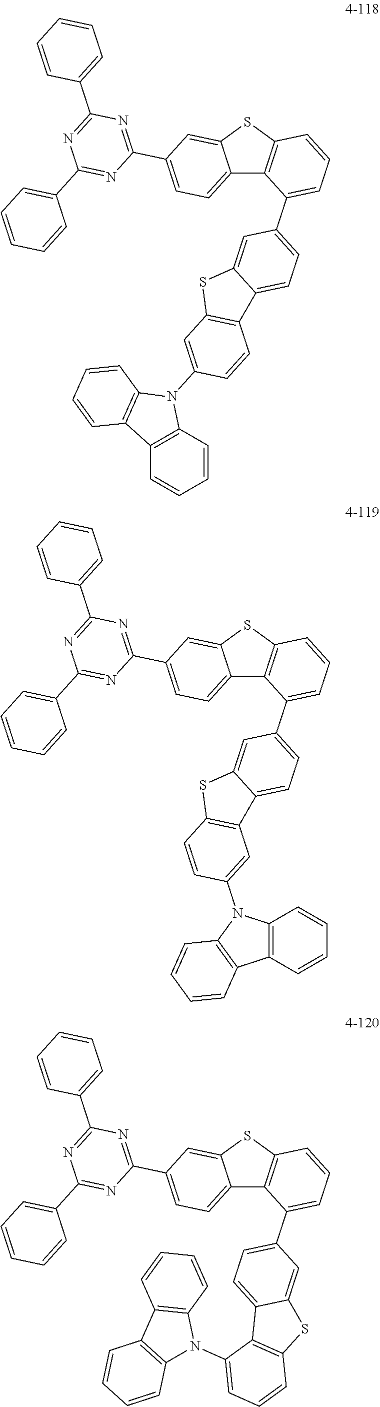 [SMILES: c1ccc(-c2nc(-c3ccccc3)nc(-c3ccc4c(c3)sc3cccc(-c5ccc6c(c5)sc5cc(-n7c8ccccc8c8ccccc87)ccc56)c34)n2)cc1 ; c1ccc(-c2nc(-c3ccccc3)nc(-c3ccc4c(c3)sc3cccc(-c5ccc6c(c5)sc5ccc(-n7c8ccccc8c8ccccc87)cc56)c34)n2)cc1 ; c1ccc(-c2nc(-c3ccccc3)nc(-c3ccc4c(c3)sc3cccc(-c5ccc6c(c5)sc5cccc(-n7c8ccccc8c8ccccc87)c56)c34)n2)cc1]

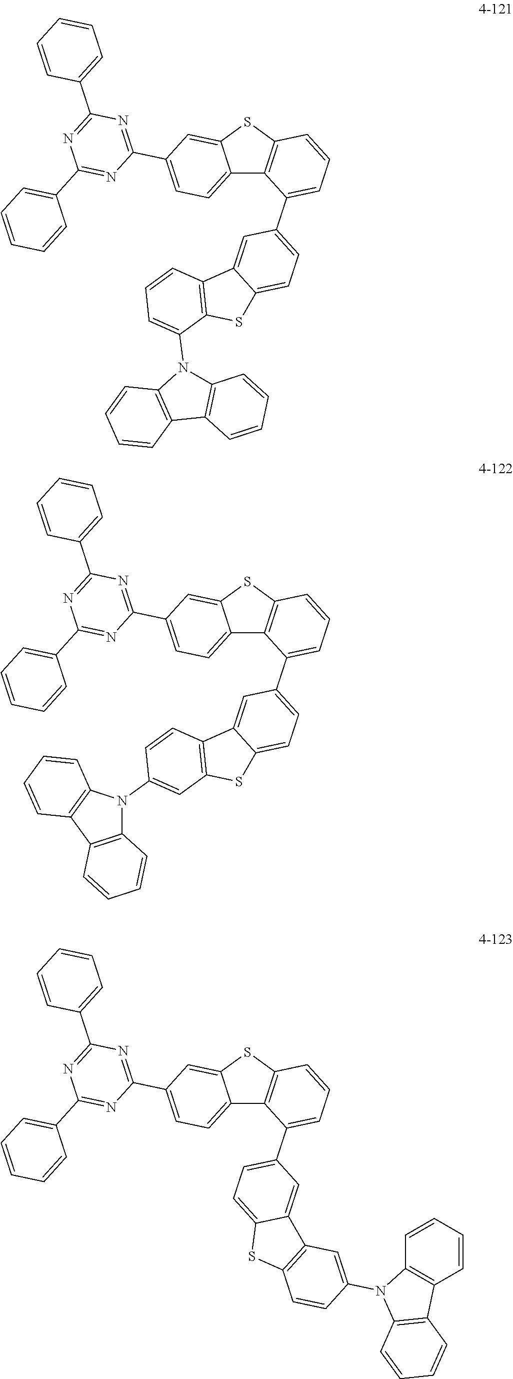 [SMILES: c1ccc(-c2nc(-c3ccccc3)nc(-c3ccc4c(c3)sc3cccc(-c5ccc6sc7c(-n8c9ccccc9c9ccccc98)cccc7c6c5)c34)n2)cc1 ; c1ccc(-c2nc(-c3ccccc3)nc(-c3ccc4c(c3)sc3cccc(-c5ccc6sc7cc(-n8c9ccccc9c9ccccc98)ccc7c6c5)c34)n2)cc1 ; c1ccc(-c2nc(-c3ccccc3)nc(-c3ccc4c(c3)sc3cccc(-c5ccc6sc7ccc(-n8c9ccccc9c9ccccc98)cc7c6c5)c34)n2)cc1]

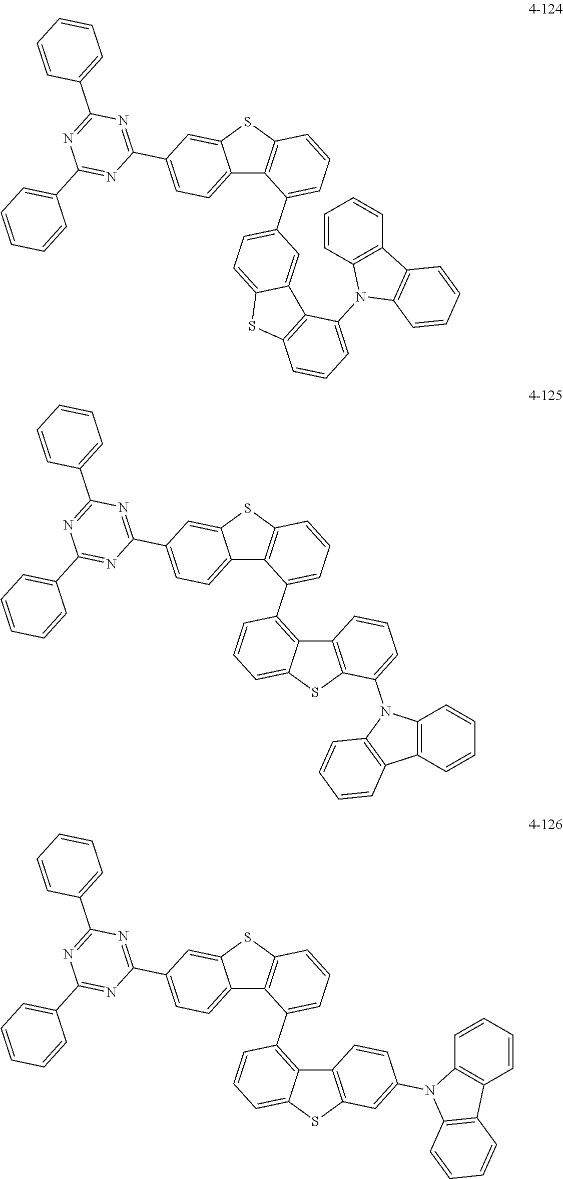 [SMILES: c1ccc(-c2nc(-c3ccccc3)nc(-c3ccc4c(c3)sc3cccc(-c5ccc6sc7cccc(-n8c9ccccc9c9ccccc98)c7c6c5)c34)n2)cc1 ; c1ccc(-c2nc(-c3ccccc3)nc(-c3ccc4c(c3)sc3cccc(-c5cccc6sc7c(-n8c9ccccc9c9ccccc98)cccc7c56)c34)n2)cc1 ; c1ccc(-c2nc(-c3ccccc3)nc(-c3ccc4c(c3)sc3cccc(-c5cccc6sc7cc(-n8c9ccccc9c9ccccc98)ccc7c56)c34)n2)cc1]

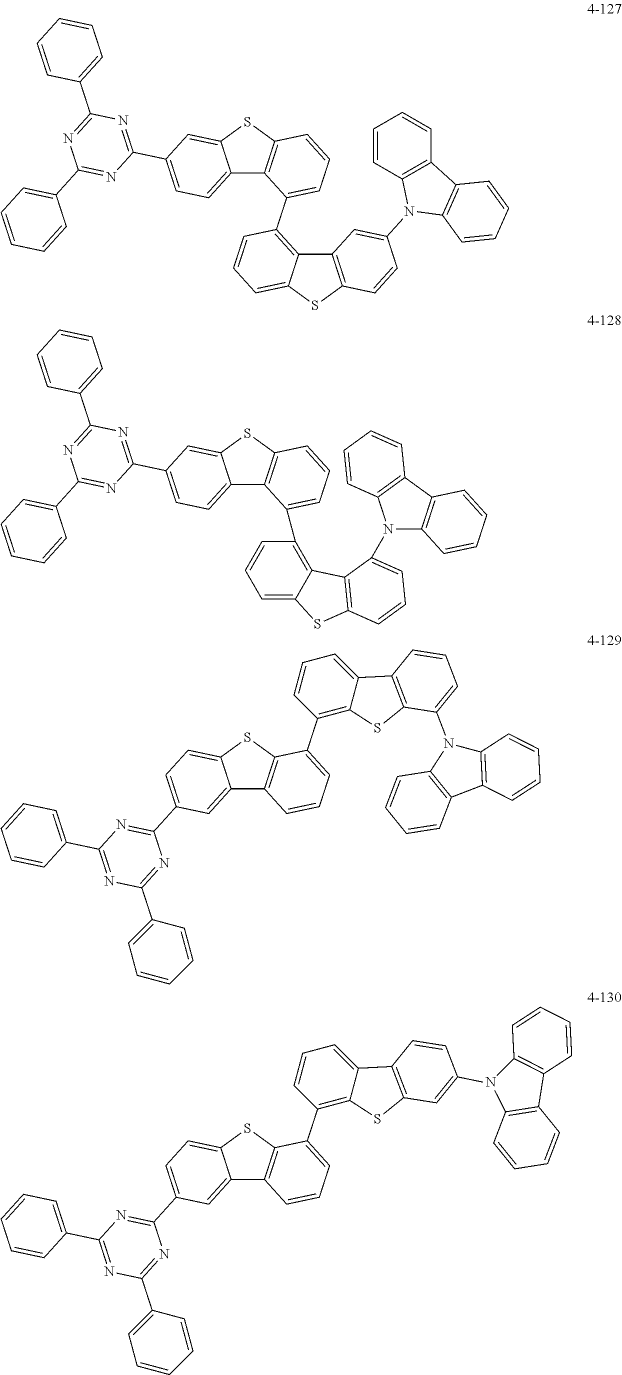

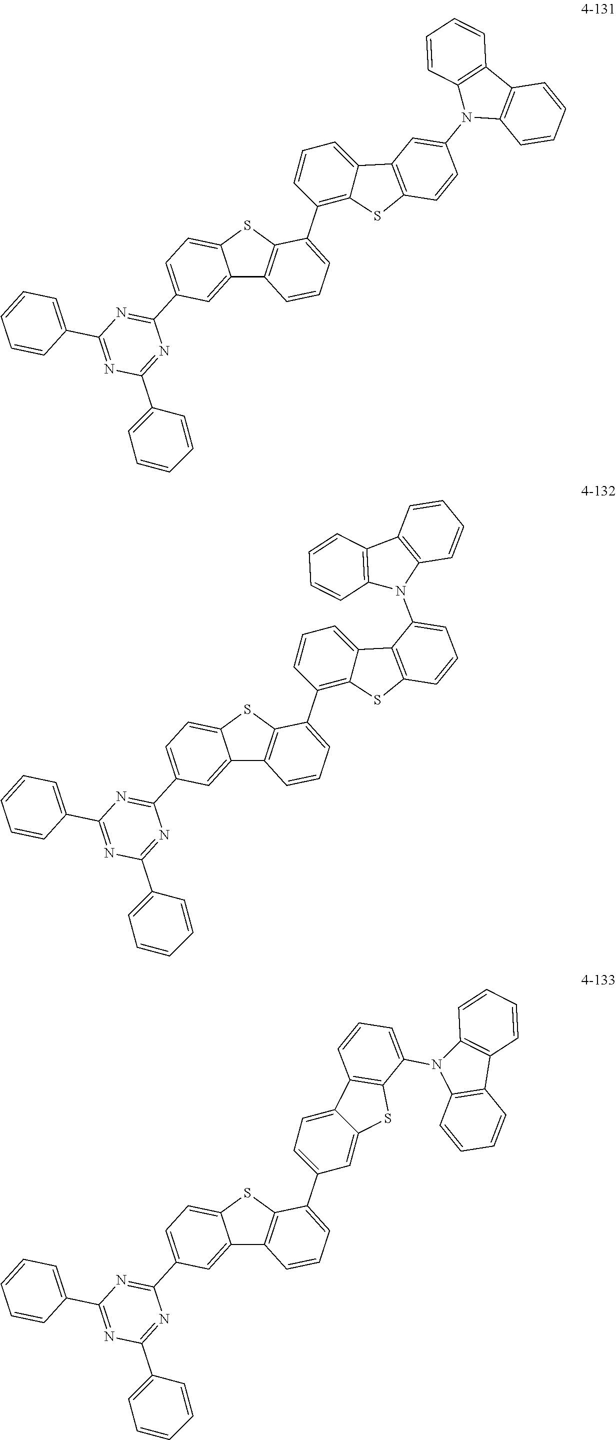 [SMILES: c1ccc(-c2nc(-c3ccccc3)nc(-c3ccc4sc5c(-c6cccc7c6sc6ccc(-n8c9ccccc9c9ccccc98)cc67)cccc5c4c3)n2)cc1 ; c1ccc(-c2nc(-c3ccccc3)nc(-c3ccc4sc5c(-c6cccc7c6sc6cccc(-n8c9ccccc9c9ccccc98)c67)cccc5c4c3)n2)cc1 ; c1ccc(-c2nc(-c3ccccc3)nc(-c3ccc4sc5c(-c6ccc7c(c6)sc6c(-n8c9ccccc9c9ccccc98)cccc67)cccc5c4c3)n2)cc1]

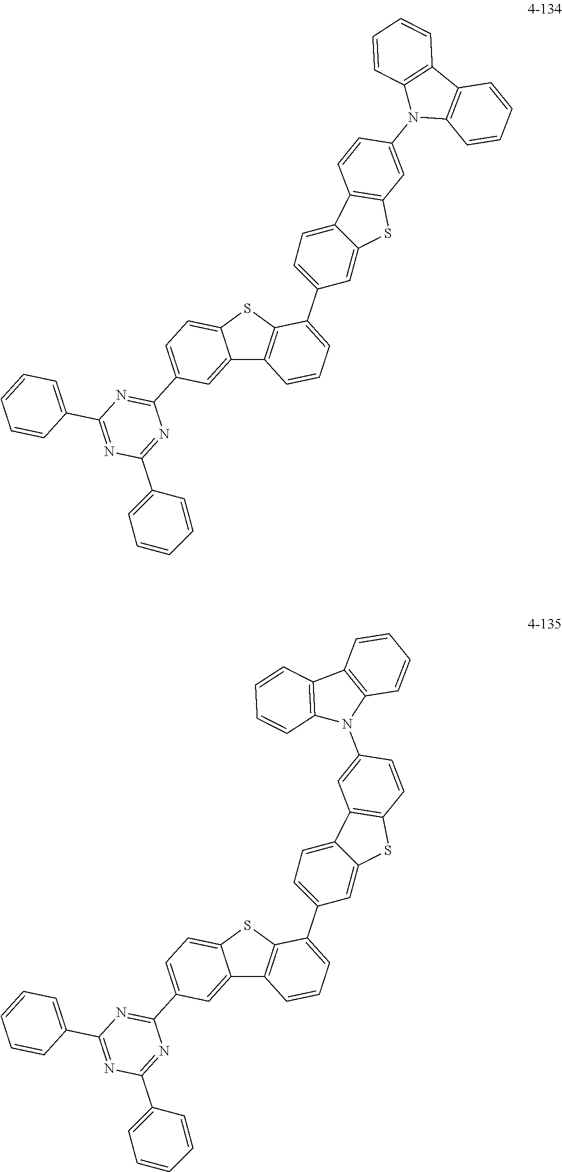 [SMILES: c1ccc(-c2nc(-c3ccccc3)nc(-c3ccc4sc5c(-c6ccc7c(c6)sc6cc(-n8c9ccccc9c9ccccc98)ccc67)cccc5c4c3)n2)cc1 ; c1ccc(-c2nc(-c3ccccc3)nc(-c3ccc4sc5c(-c6ccc7c(c6)sc6ccc(-n8c9ccccc9c9ccccc98)cc67)cccc5c4c3)n2)cc1]

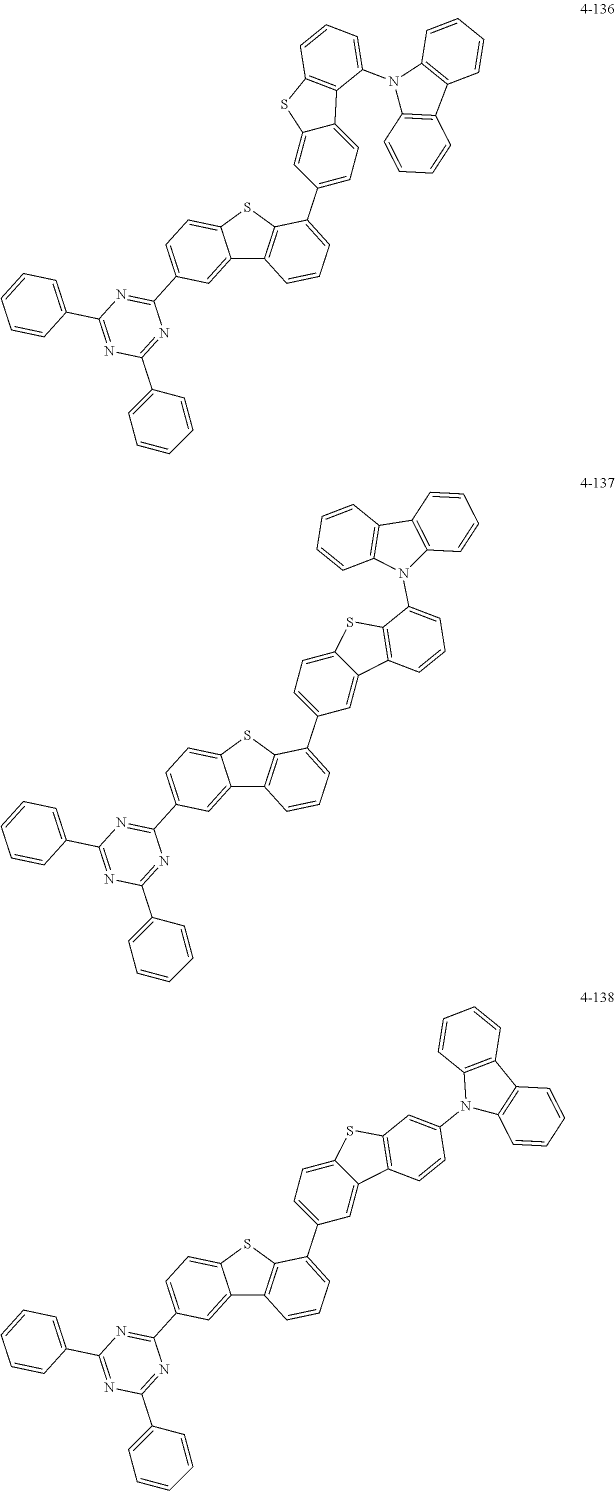 [SMILES: c1ccc(-c2nc(-c3ccccc3)nc(-c3ccc4sc5c(-c6ccc7c(c6)sc6cccc(-n8c9ccccc9c9ccccc98)c67)cccc5c4c3)n2)cc1 ; c1ccc(-c2nc(-c3ccccc3)nc(-c3ccc4sc5c(-c6ccc7sc8c(-n9c%10ccccc%10c%10ccccc%109)cccc8c7c6)cccc5c4c3)n2)cc1 ; c1ccc(-c2nc(-c3ccccc3)nc(-c3ccc4sc5c(-c6ccc7sc8cc(-n9c%10ccccc%10c%10ccccc%109)ccc8c7c6)cccc5c4c3)n2)cc1]

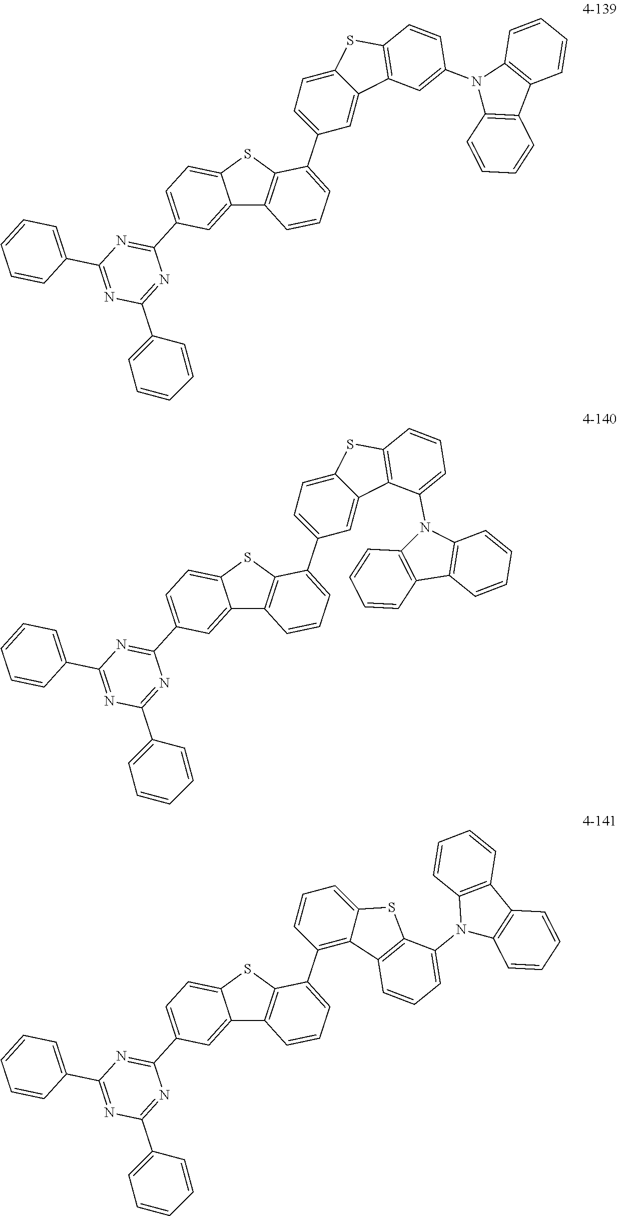 [SMILES: c1ccc(-c2nc(-c3ccccc3)nc(-c3ccc4sc5c(-c6ccc7sc8ccc(-n9c%10ccccc%10c%10ccccc%109)cc8c7c6)cccc5c4c3)n2)cc1 ; c1ccc(-c2nc(-c3ccccc3)nc(-c3ccc4sc5c(-c6ccc7sc8cccc(-n9c%10ccccc%10c%10ccccc%109)c8c7c6)cccc5c4c3)n2)cc1 ; c1ccc(-c2nc(-c3ccccc3)nc(-c3ccc4sc5c(-c6cccc7sc8c(-n9c%10ccccc%10c%10ccccc%109)cccc8c67)cccc5c4c3)n2)cc1]

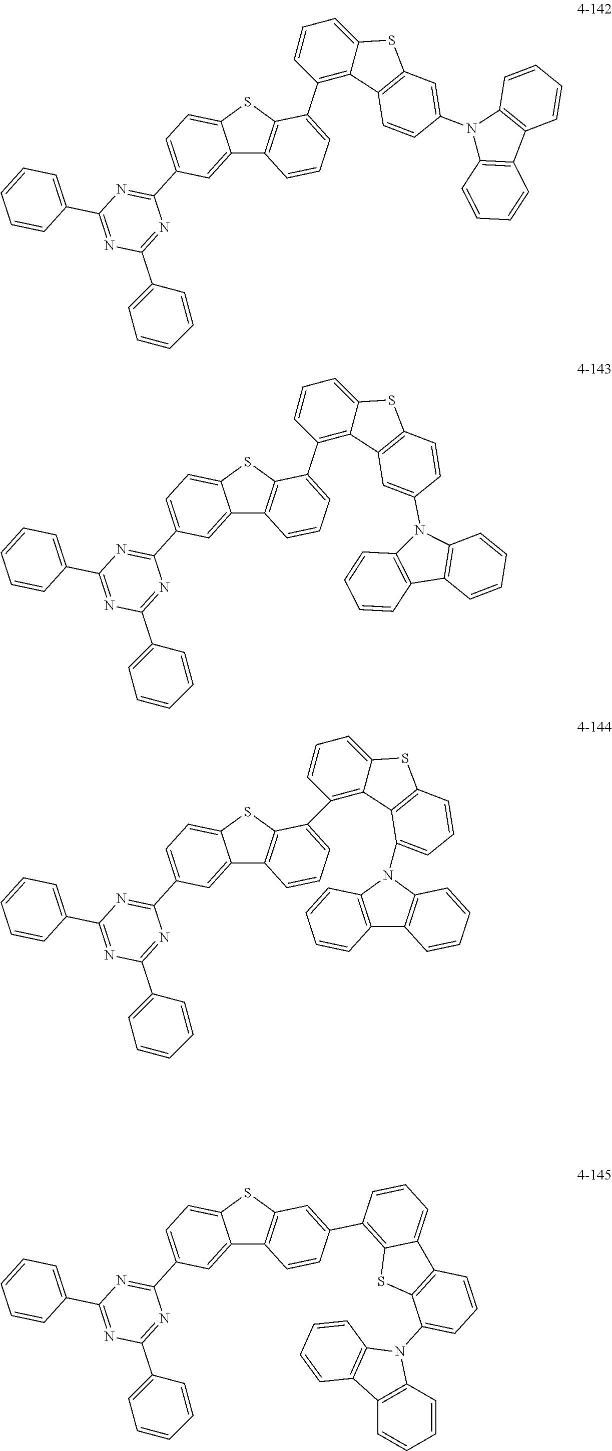 [SMILES: c1ccc(-c2nc(-c3ccccc3)nc(-c3ccc4sc5c(-c6cccc7sc8cc(-n9c%10ccccc%10c%10ccccc%109)ccc8c67)cccc5c4c3)n2)cc1 ; c1ccc(-c2nc(-c3ccccc3)nc(-c3ccc4sc5c(-c6cccc7sc8ccc(-n9c%10ccccc%10c%10ccccc%109)cc8c67)cccc5c4c3)n2)cc1 ; c1ccc(-c2nc(-c3ccccc3)nc(-c3ccc4sc5c(-c6cccc7sc8cccc(-n9c%10ccccc%10c%10ccccc%109)c8c67)cccc5c4c3)n2)cc1 ; c1ccc(-c2nc(-c3ccccc3)nc(-c3ccc4sc5cc(-c6cccc7c6sc6c(-n8c9ccccc9c9ccccc98)cccc67)ccc5c4c3)n2)cc1]

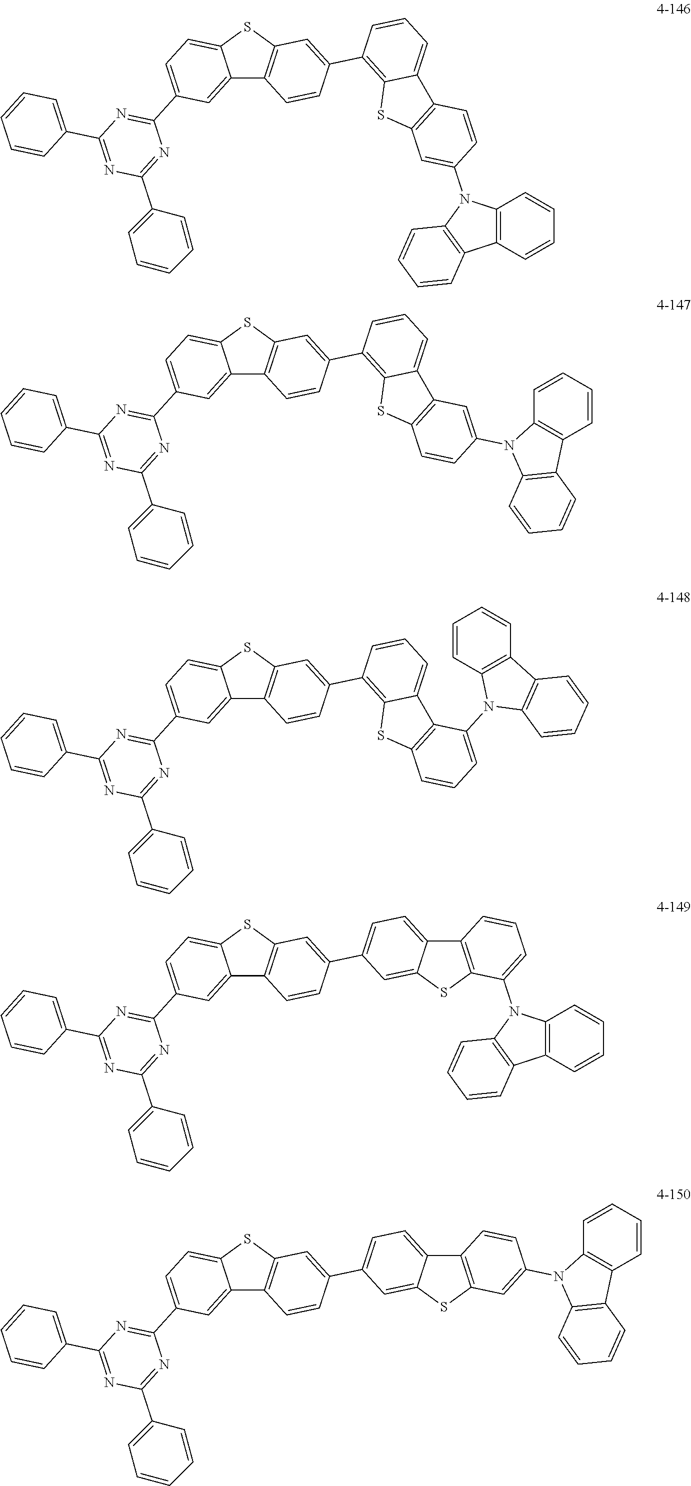

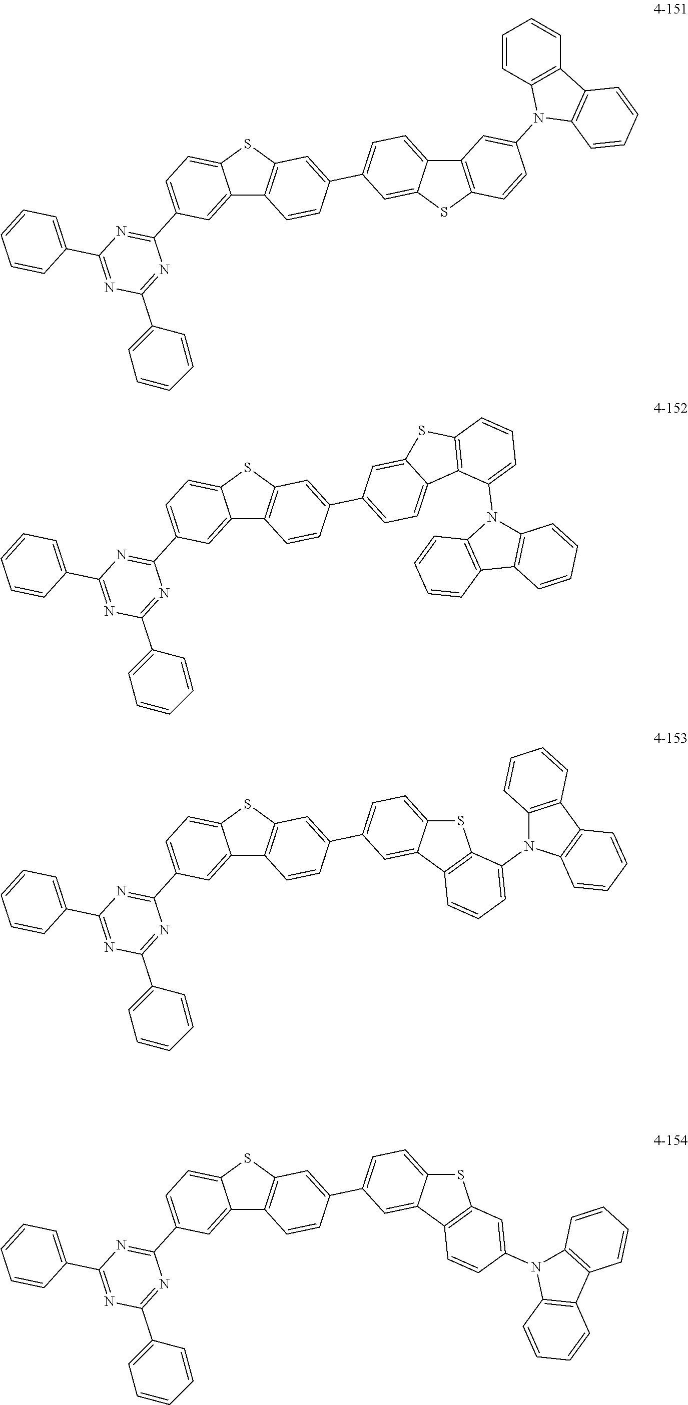 [SMILES: c1ccc(-c2nc(-c3ccccc3)nc(-c3ccc4sc5cc(-c6ccc7c(c6)sc6ccc(-n8c9ccccc9c9ccccc98)cc67)ccc5c4c3)n2)cc1 ; c1ccc(-c2nc(-c3ccccc3)nc(-c3ccc4sc5cc(-c6ccc7c(c6)sc6cccc(-n8c9ccccc9c9ccccc98)c67)ccc5c4c3)n2)cc1 ; c1ccc(-c2nc(-c3ccccc3)nc(-c3ccc4sc5cc(-c6ccc7sc8c(-n9c%10ccccc%10c%10ccccc%109)cccc8c7c6)ccc5c4c3)n2)cc1 ; c1ccc(-c2nc(-c3ccccc3)nc(-c3ccc4sc5cc(-c6ccc7sc8cc(-n9c%10ccccc%10c%10ccccc%109)ccc8c7c6)ccc5c4c3)n2)cc1]

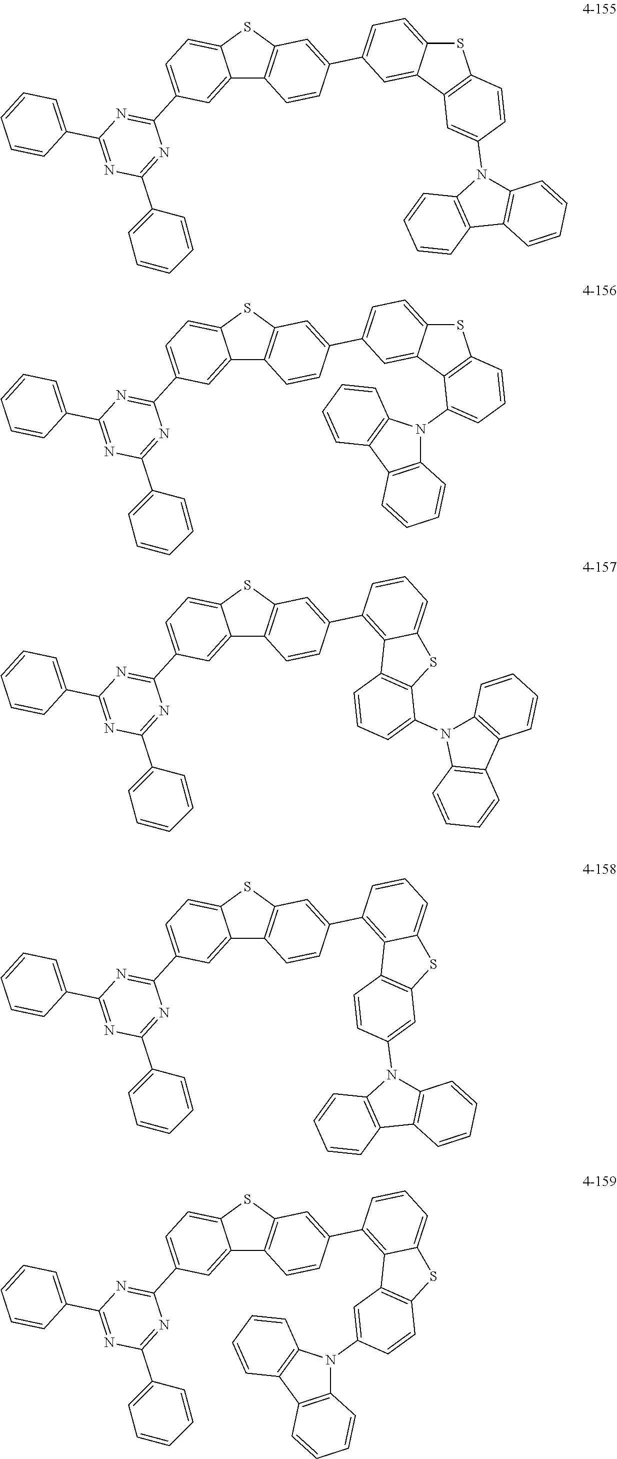

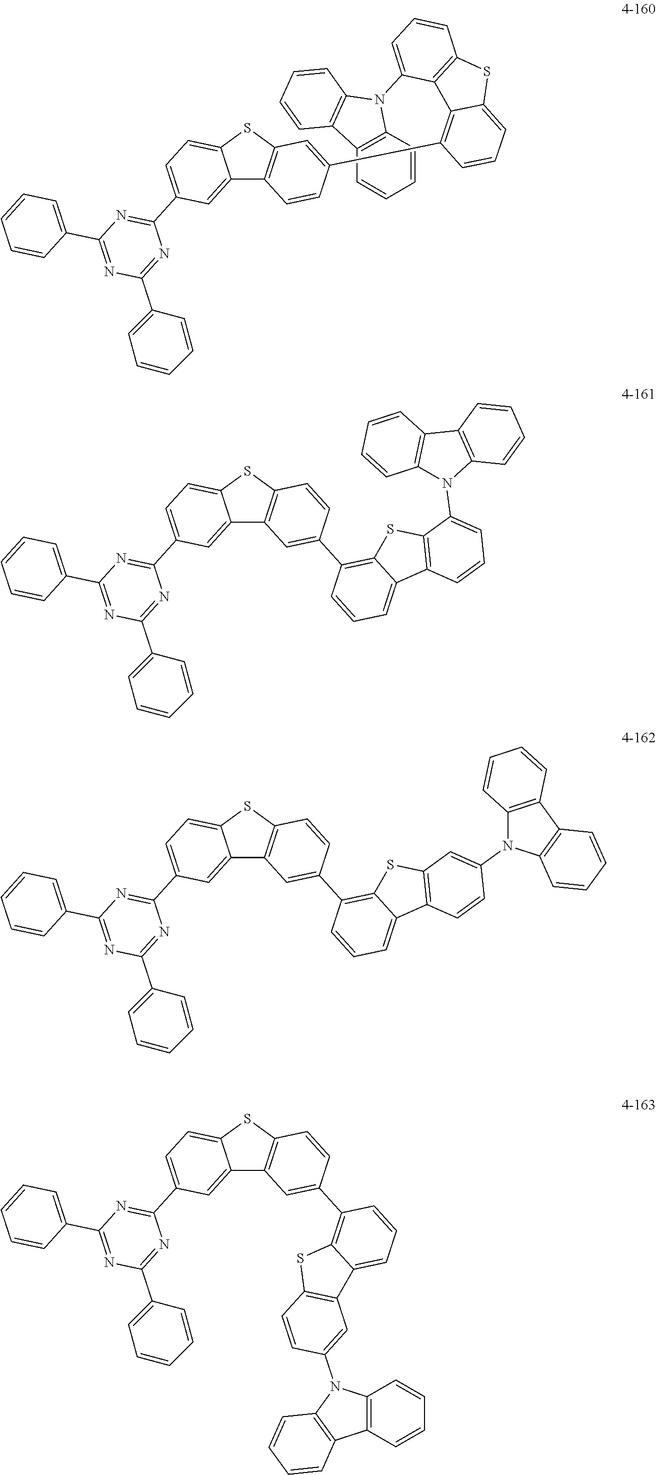 [SMILES: c1ccc(-c2nc(-c3ccccc3)nc(-c3ccc4sc5cc(-c6cccc7sc8cccc(-n9c%10ccccc%10c%10ccccc%109)c8c67)ccc5c4c3)n2)cc1 ; c1ccc(-c2nc(-c3ccccc3)nc(-c3ccc4sc5ccc(-c6cccc7c6sc6c(-n8c9ccccc9c9ccccc98)cccc67)cc5c4c3)n2)cc1 ; c1ccc(-c2nc(-c3ccccc3)nc(-c3ccc4sc5ccc(-c6cccc7c6sc6cc(-n8c9ccccc9c9ccccc98)ccc67)cc5c4c3)n2)cc1 ; c1ccc(-c2nc(-c3ccccc3)nc(-c3ccc4sc5ccc(-c6cccc7c6sc6ccc(-n8c9ccccc9c9ccccc98)cc67)cc5c4c3)n2)cc1]

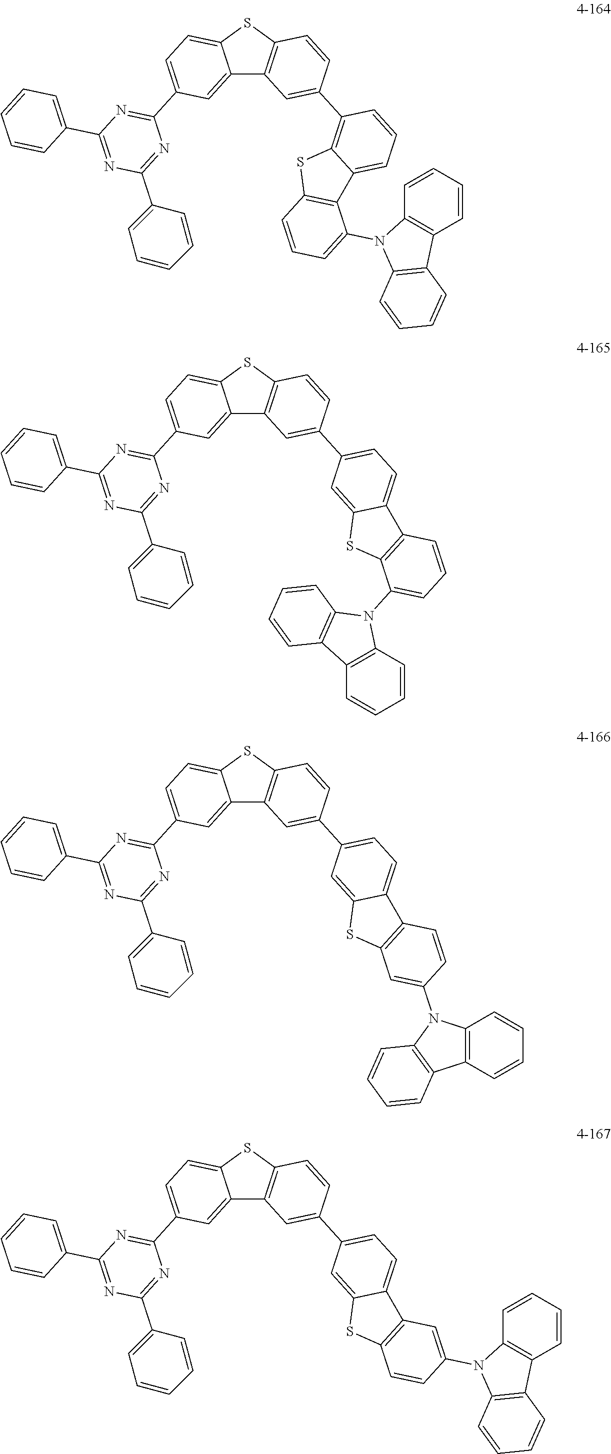 [SMILES: c1ccc(-c2nc(-c3ccccc3)nc(-c3ccc4sc5ccc(-c6cccc7c6sc6cccc(-n8c9ccccc9c9ccccc98)c67)cc5c4c3)n2)cc1 ; c1ccc(-c2nc(-c3ccccc3)nc(-c3ccc4sc5ccc(-c6ccc7c(c6)sc6c(-n8c9ccccc9c9ccccc98)cccc67)cc5c4c3)n2)cc1 ; c1ccc(-c2nc(-c3ccccc3)nc(-c3ccc4sc5ccc(-c6ccc7c(c6)sc6cc(-n8c9ccccc9c9ccccc98)ccc67)cc5c4c3)n2)cc1 ; c1ccc(-c2nc(-c3ccccc3)nc(-c3ccc4sc5ccc(-c6ccc7c(c6)sc6ccc(-n8c9ccccc9c9ccccc98)cc67)cc5c4c3)n2)cc1]

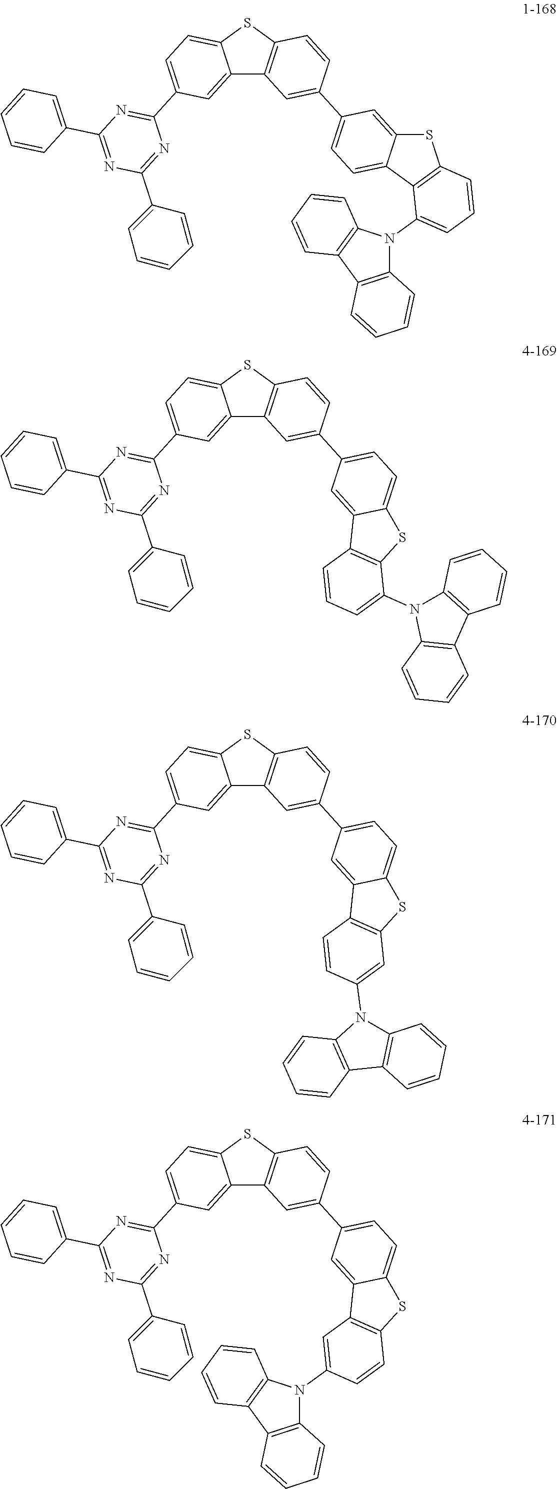 [SMILES: c1ccc(-c2nc(-c3ccccc3)nc(-c3ccc4sc5ccc(-c6ccc7c(c6)sc6cccc(-n8c9ccccc9c9ccccc98)c67)cc5c4c3)n2)cc1 ; c1ccc(-c2nc(-c3ccccc3)nc(-c3ccc4sc5ccc(-c6ccc7sc8c(-n9c%10ccccc%10c%10ccccc%109)cccc8c7c6)cc5c4c3)n2)cc1 ; c1ccc(-c2nc(-c3ccccc3)nc(-c3ccc4sc5ccc(-c6ccc7sc8cc(-n9c%10ccccc%10c%10ccccc%109)ccc8c7c6)cc5c4c3)n2)cc1 ; c1ccc(-c2nc(-c3ccccc3)nc(-c3ccc4sc5ccc(-c6ccc7sc8ccc(-n9c%10ccccc%10c%10ccccc%109)cc8c7c6)cc5c4c3)n2)cc1]

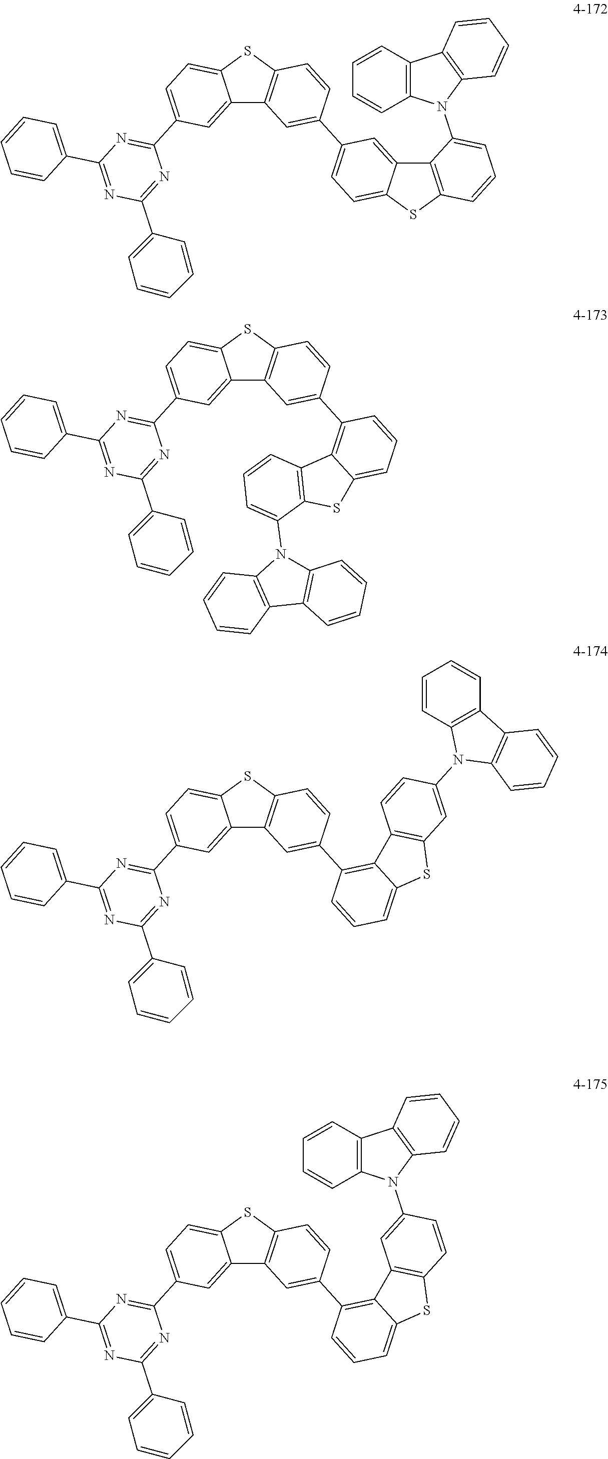 [SMILES: c1ccc(-c2nc(-c3ccccc3)nc(-c3ccc4sc5ccc(-c6ccc7sc8cccc(-n9c%10ccccc%10c%10ccccc%109)c8c7c6)cc5c4c3)n2)cc1 ; c1ccc(-c2nc(-c3ccccc3)nc(-c3ccc4sc5ccc(-c6cccc7sc8c(-n9c%10ccccc%10c%10ccccc%109)cccc8c67)cc5c4c3)n2)cc1 ; c1ccc(-c2nc(-c3ccccc3)nc(-c3ccc4sc5ccc(-c6cccc7sc8cc(-n9c%10ccccc%10c%10ccccc%109)ccc8c67)cc5c4c3)n2)cc1 ; c1ccc(-c2nc(-c3ccccc3)nc(-c3ccc4sc5ccc(-c6cccc7sc8ccc(-n9c%10ccccc%10c%10ccccc%109)cc8c67)cc5c4c3)n2)cc1]

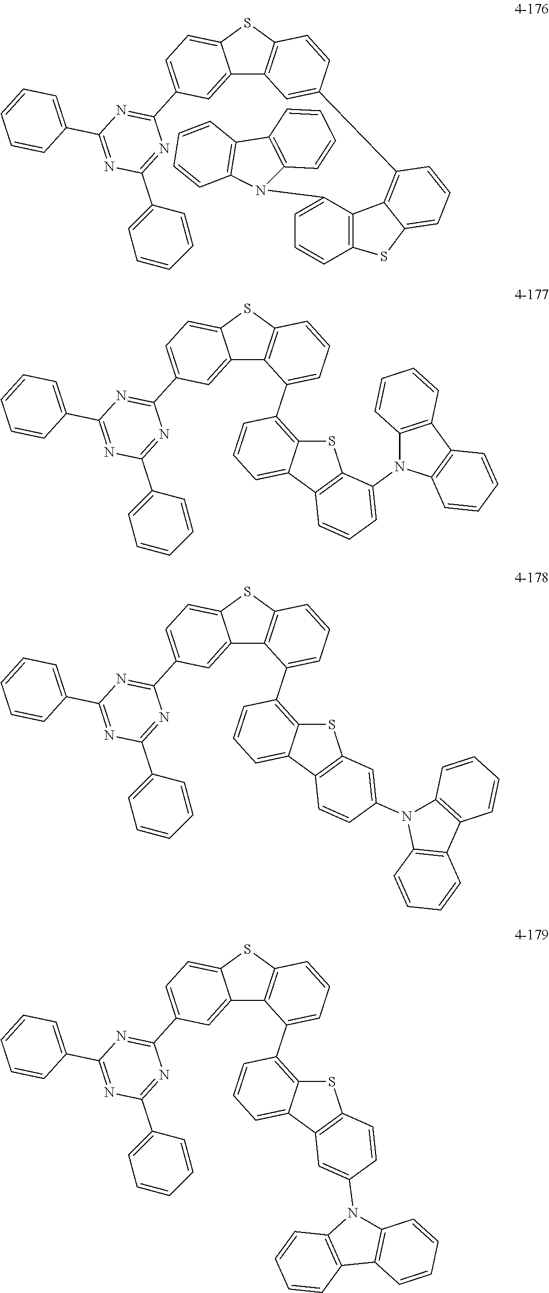 [SMILES: c1ccc(-c2nc(-c3ccccc3)nc(-c3ccc4sc5ccc(-c6cccc7sc8cccc(-n9c%10ccccc%10c%10ccccc%109)c8c67)cc5c4c3)n2)cc1 ; c1ccc(-c2nc(-c3ccccc3)nc(-c3ccc4sc5cccc(-c6cccc7c6sc6c(-n8c9ccccc9c9ccccc98)cccc67)c5c4c3)n2)cc1 ; c1ccc(-c2nc(-c3ccccc3)nc(-c3ccc4sc5cccc(-c6cccc7c6sc6cc(-n8c9ccccc9c9ccccc98)ccc67)c5c4c3)n2)cc1 ; c1ccc(-c2nc(-c3ccccc3)nc(-c3ccc4sc5cccc(-c6cccc7c6sc6ccc(-n8c9ccccc9c9ccccc98)cc67)c5c4c3)n2)cc1]

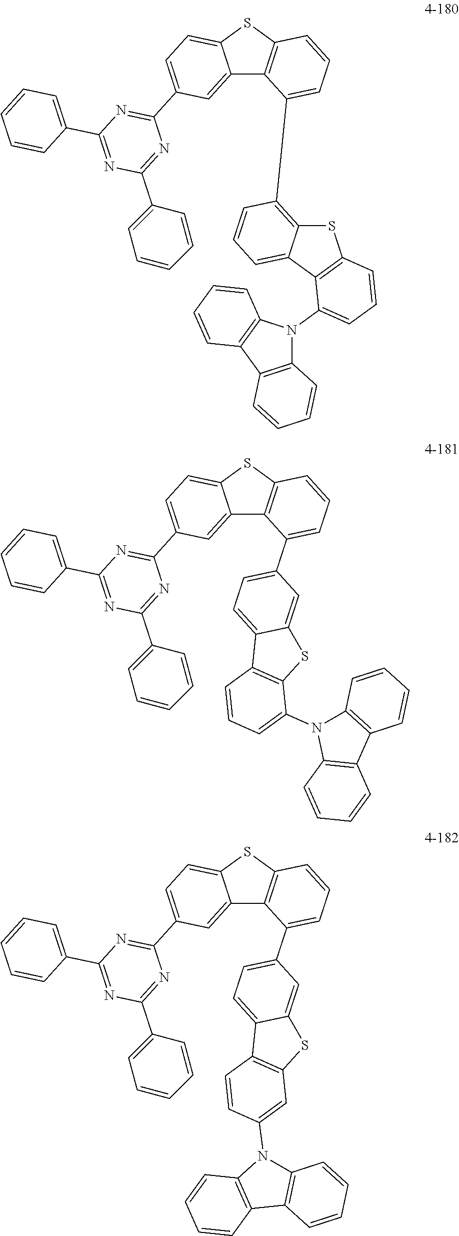

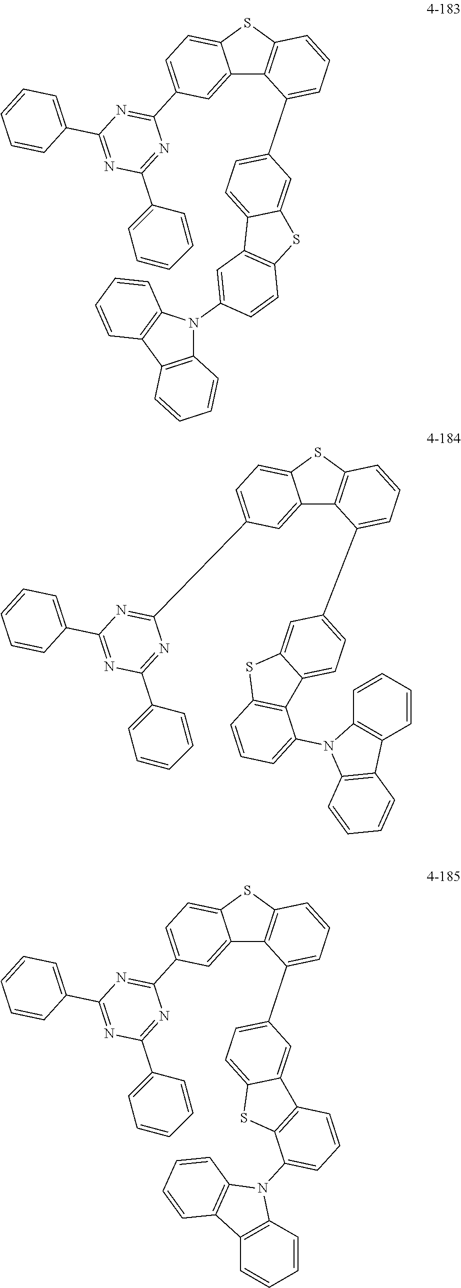 [SMILES: c1ccc(-c2nc(-c3ccccc3)nc(-c3ccc4sc5cccc(-c6ccc7c(c6)sc6ccc(-n8c9ccccc9c9ccccc98)cc67)c5c4c3)n2)cc1 ; c1ccc(-c2nc(-c3ccccc3)nc(-c3ccc4sc5cccc(-c6ccc7c(c6)sc6cccc(-n8c9ccccc9c9ccccc98)c67)c5c4c3)n2)cc1 ; c1ccc(-c2nc(-c3ccccc3)nc(-c3ccc4sc5cccc(-c6ccc7sc8c(-n9c%10ccccc%10c%10ccccc%109)cccc8c7c6)c5c4c3)n2)cc1]

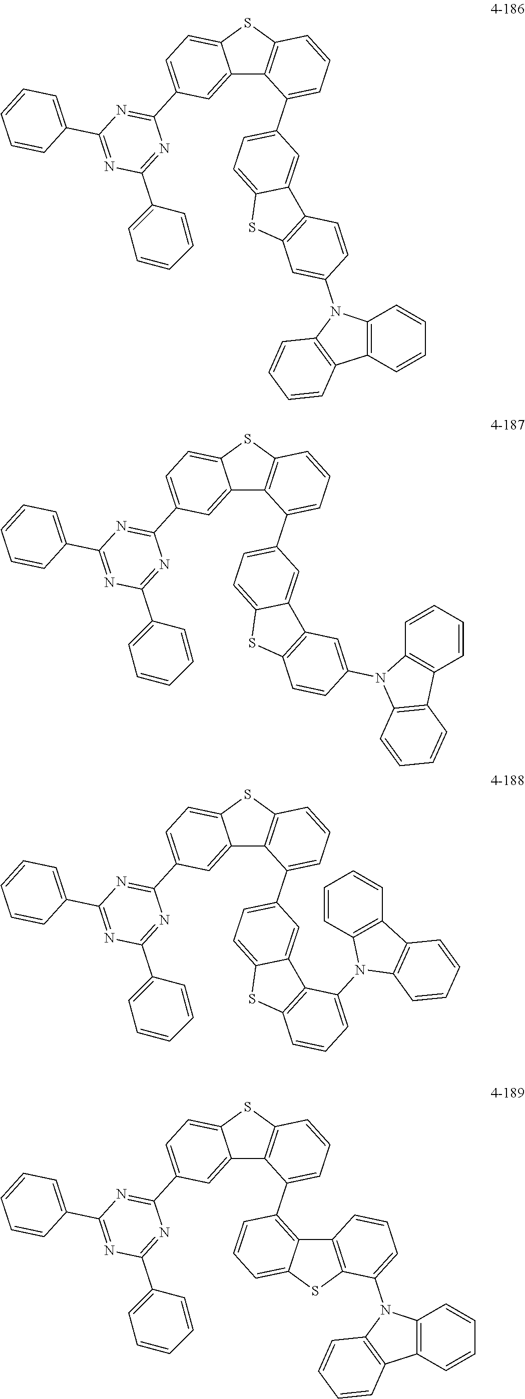 [SMILES: c1ccc(-c2nc(-c3ccccc3)nc(-c3ccc4sc5cccc(-c6ccc7sc8cc(-n9c%10ccccc%10c%10ccccc%109)ccc8c7c6)c5c4c3)n2)cc1 ; c1ccc(-c2nc(-c3ccccc3)nc(-c3ccc4sc5cccc(-c6ccc7sc8ccc(-n9c%10ccccc%10c%10ccccc%109)cc8c7c6)c5c4c3)n2)cc1 ; c1ccc(-c2nc(-c3ccccc3)nc(-c3ccc4sc5cccc(-c6ccc7sc8cccc(-n9c%10ccccc%10c%10ccccc%109)c8c7c6)c5c4c3)n2)cc1 ; c1ccc(-c2nc(-c3ccccc3)nc(-c3ccc4sc5cccc(-c6cccc7sc8c(-n9c%10ccccc%10c%10ccccc%109)cccc8c67)c5c4c3)n2)cc1]

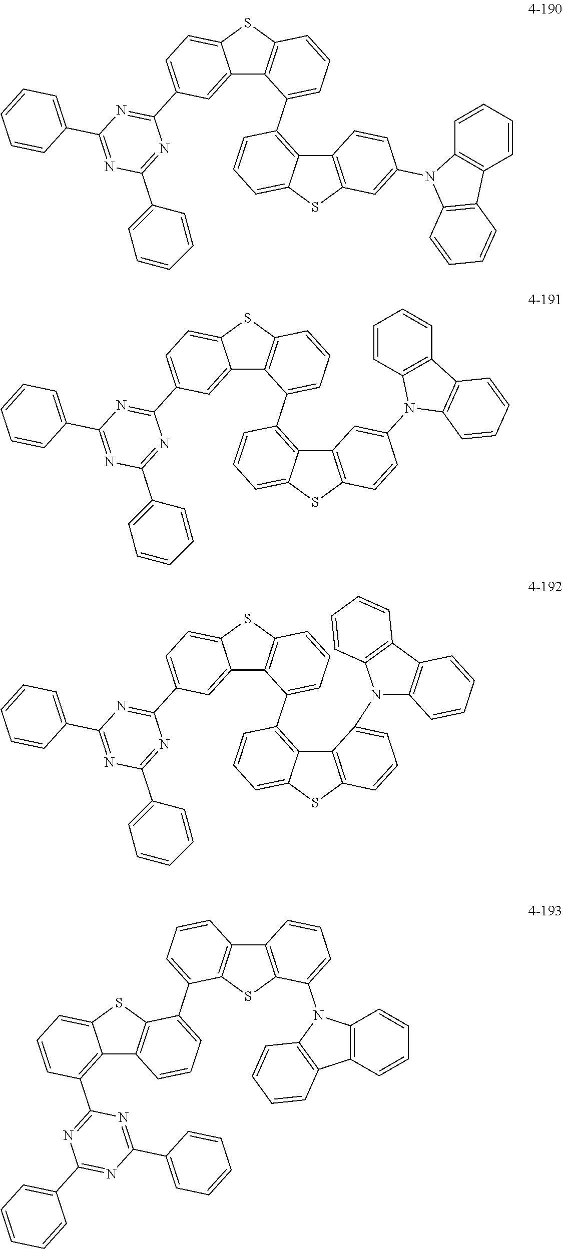 [SMILES: c1ccc(-c2nc(-c3ccccc3)nc(-c3ccc4sc5cccc(-c6cccc7sc8cc(-n9c%10ccccc%10c%10ccccc%109)ccc8c67)c5c4c3)n2)cc1 ; c1ccc(-c2nc(-c3ccccc3)nc(-c3ccc4sc5cccc(-c6cccc7sc8ccc(-n9c%10ccccc%10c%10ccccc%109)cc8c67)c5c4c3)n2)cc1 ; c1ccc(-c2nc(-c3ccccc3)nc(-c3ccc4sc5cccc(-c6cccc7sc8cccc(-n9c%10ccccc%10c%10ccccc%109)c8c67)c5c4c3)n2)cc1 ; c1ccc(-c2nc(-c3ccccc3)nc(-c3cccc4sc5c(-c6cccc7c6sc6c(-n8c9ccccc9c9ccccc98)cccc67)cccc5c34)n2)cc1]

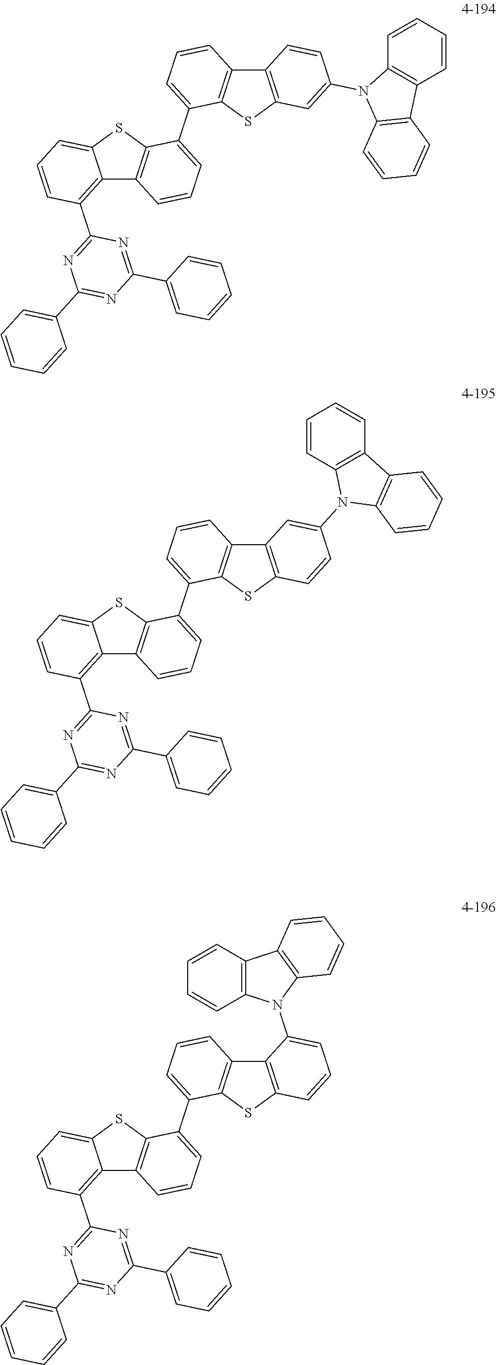 [SMILES: c1ccc(-c2nc(-c3ccccc3)nc(-c3cccc4sc5c(-c6cccc7c6sc6cc(-n8c9ccccc9c9ccccc98)ccc67)cccc5c34)n2)cc1 ; c1ccc(-c2nc(-c3ccccc3)nc(-c3cccc4sc5c(-c6cccc7c6sc6ccc(-n8c9ccccc9c9ccccc98)cc67)cccc5c34)n2)cc1 ; c1ccc(-c2nc(-c3ccccc3)nc(-c3cccc4sc5c(-c6cccc7c6sc6cccc(-n8c9ccccc9c9ccccc98)c67)cccc5c34)n2)cc1]

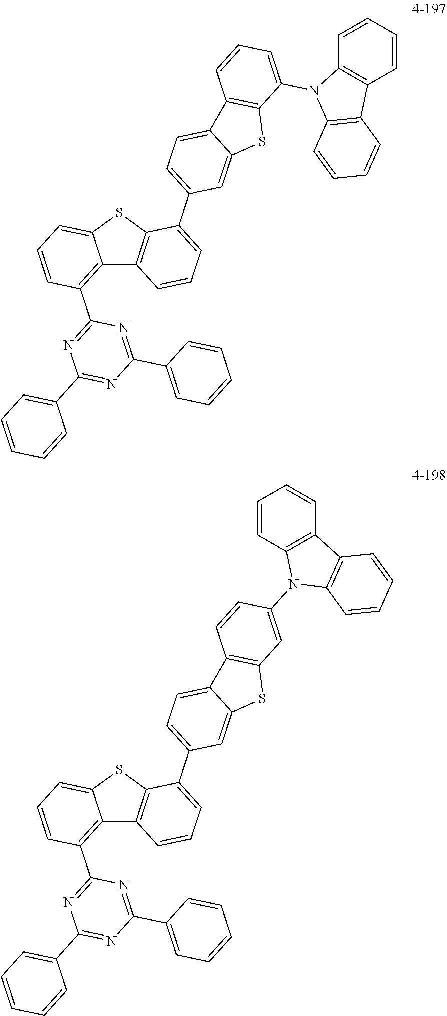 [SMILES: c1ccc(-c2nc(-c3ccccc3)nc(-c3cccc4sc5c(-c6ccc7c(c6)sc6c(-n8c9ccccc9c9ccccc98)cccc67)cccc5c34)n2)cc1 ; c1ccc(-c2nc(-c3ccccc3)nc(-c3cccc4sc5c(-c6ccc7c(c6)sc6cc(-n8c9ccccc9c9ccccc98)ccc67)cccc5c34)n2)cc1]

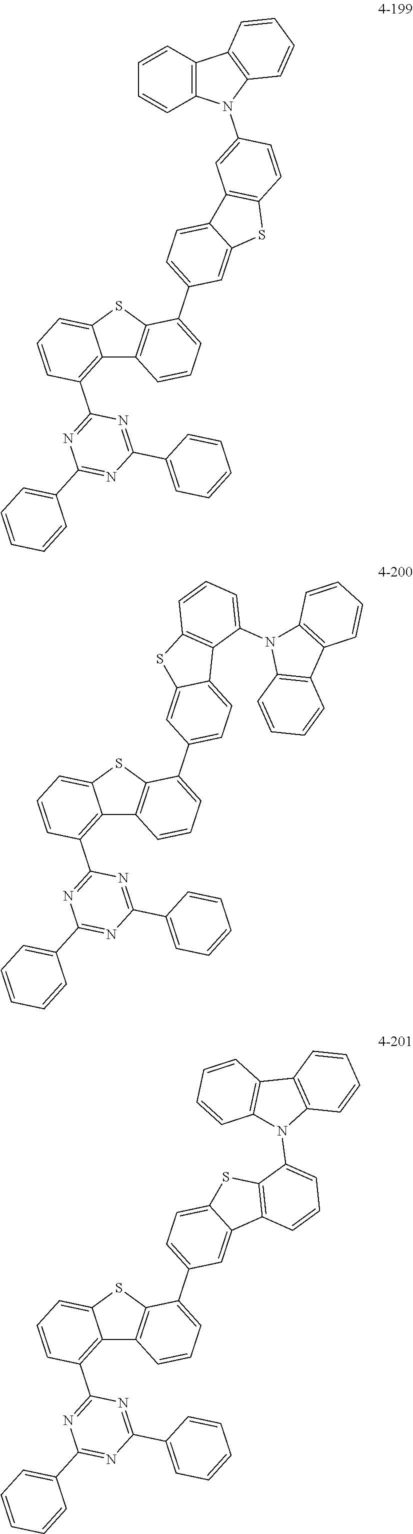 [SMILES: c1ccc(-c2nc(-c3ccccc3)nc(-c3cccc4sc5c(-c6ccc7c(c6)sc6ccc(-n8c9ccccc9c9ccccc98)cc67)cccc5c34)n2)cc1 ; c1ccc(-c2nc(-c3ccccc3)nc(-c3cccc4sc5c(-c6ccc7c(c6)sc6cccc(-n8c9ccccc9c9ccccc98)c67)cccc5c34)n2)cc1 ; c1ccc(-c2nc(-c3ccccc3)nc(-c3cccc4sc5c(-c6ccc7sc8c(-n9c%10ccccc%10c%10ccccc%109)cccc8c7c6)cccc5c34)n2)cc1]

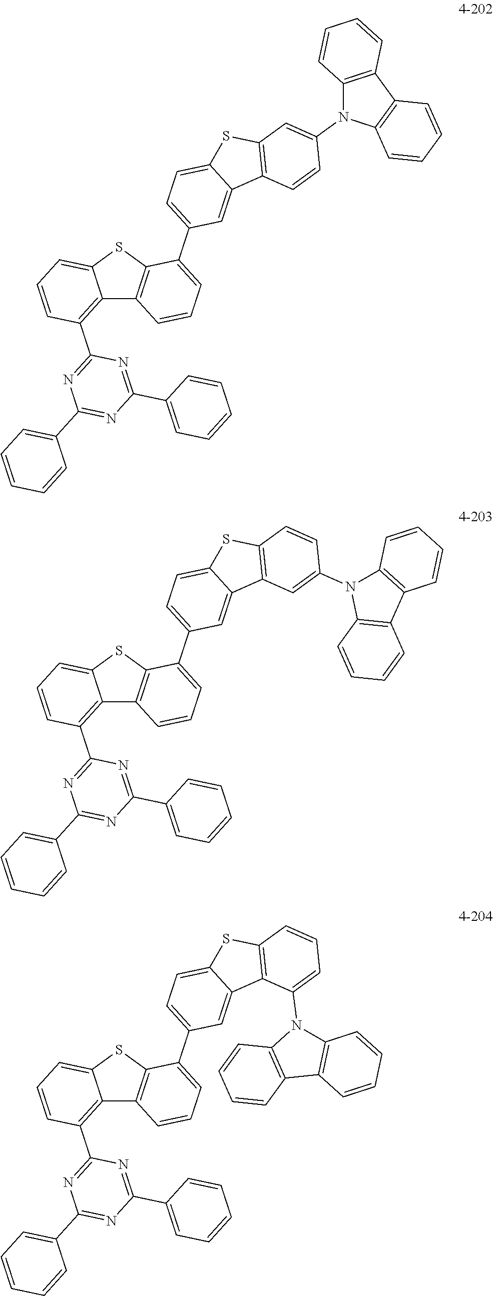 [SMILES: c1ccc(-c2nc(-c3ccccc3)nc(-c3cccc4sc5c(-c6ccc7sc8cc(-n9c%10ccccc%10c%10ccccc%109)ccc8c7c6)cccc5c34)n2)cc1 ; c1ccc(-c2nc(-c3ccccc3)nc(-c3cccc4sc5c(-c6ccc7sc8ccc(-n9c%10ccccc%10c%10ccccc%109)cc8c7c6)cccc5c34)n2)cc1 ; c1ccc(-c2nc(-c3ccccc3)nc(-c3cccc4sc5c(-c6ccc7sc8cccc(-n9c%10ccccc%10c%10ccccc%109)c8c7c6)cccc5c34)n2)cc1]

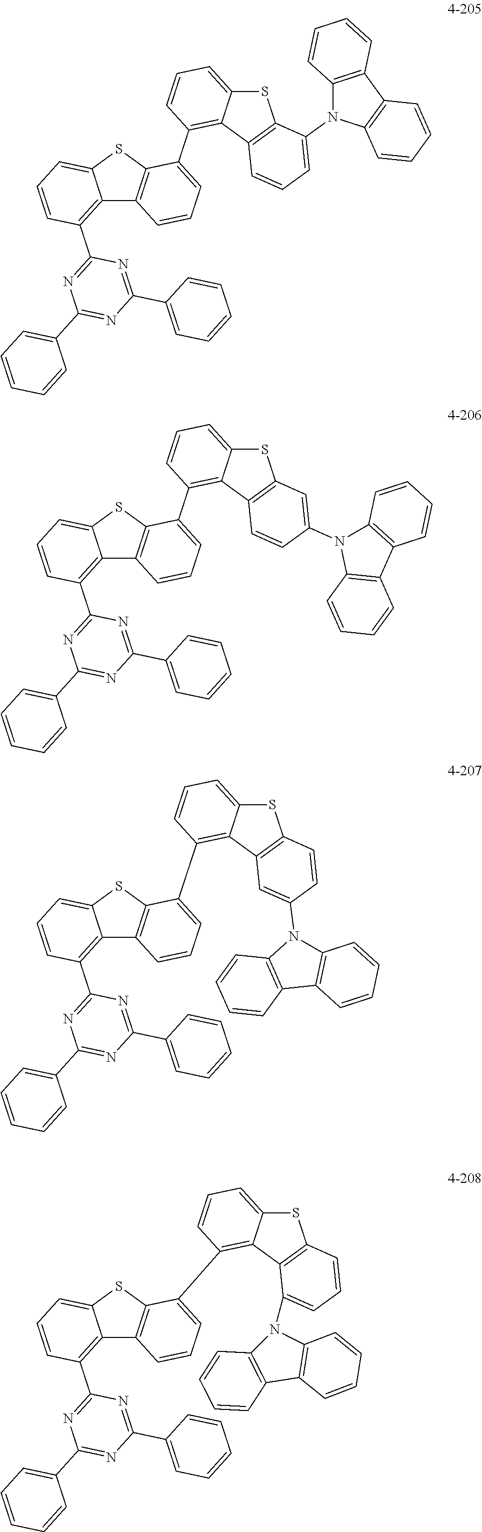 [SMILES: c1ccc(-c2nc(-c3ccccc3)nc(-c3cccc4sc5c(-c6cccc7sc8c(-n9c%10ccccc%10c%10ccccc%109)cccc8c67)cccc5c34)n2)cc1 ; c1ccc(-c2nc(-c3ccccc3)nc(-c3cccc4sc5c(-c6cccc7sc8cc(-n9c%10ccccc%10c%10ccccc%109)ccc8c67)cccc5c34)n2)cc1 ; c1ccc(-c2nc(-c3ccccc3)nc(-c3cccc4sc5c(-c6cccc7sc8ccc(-n9c%10ccccc%10c%10ccccc%109)cc8c67)cccc5c34)n2)cc1 ; c1ccc(-c2nc(-c3ccccc3)nc(-c3cccc4sc5c(-c6cccc7sc8cccc(-n9c%10ccccc%10c%10ccccc%109)c8c67)cccc5c34)n2)cc1]

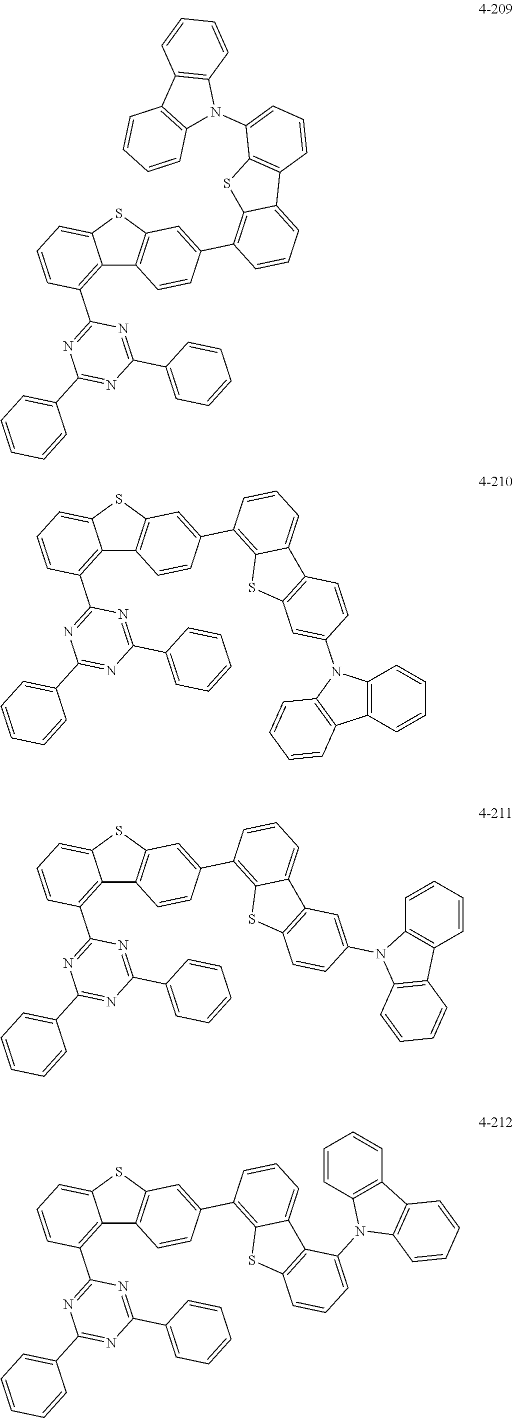 [SMILES: c1ccc(-c2nc(-c3ccccc3)nc(-c3cccc4sc5cc(-c6cccc7c6sc6c(-n8c9ccccc9c9ccccc98)cccc67)ccc5c34)n2)cc1 ; c1ccc(-c2nc(-c3ccccc3)nc(-c3cccc4sc5cc(-c6cccc7c6sc6cc(-n8c9ccccc9c9ccccc98)ccc67)ccc5c34)n2)cc1 ; c1ccc(-c2nc(-c3ccccc3)nc(-c3cccc4sc5cc(-c6cccc7c6sc6ccc(-n8c9ccccc9c9ccccc98)cc67)ccc5c34)n2)cc1 ; c1ccc(-c2nc(-c3ccccc3)nc(-c3cccc4sc5cc(-c6cccc7c6sc6cccc(-n8c9ccccc9c9ccccc98)c67)ccc5c34)n2)cc1]

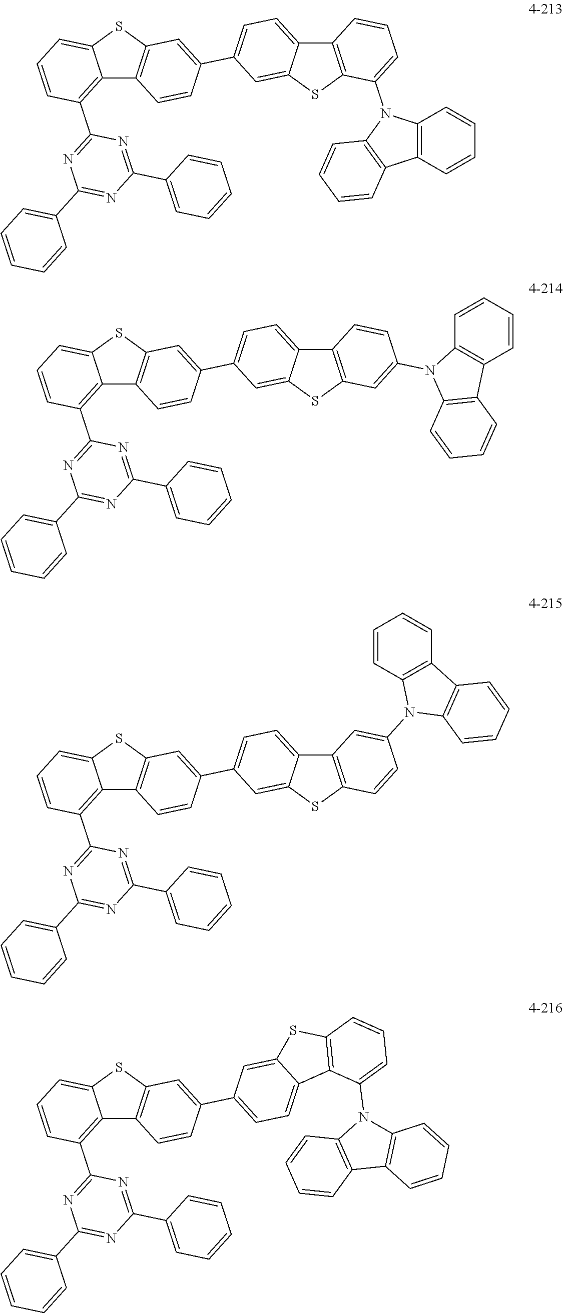 [SMILES: c1ccc(-c2nc(-c3ccccc3)nc(-c3cccc4sc5cc(-c6ccc7c(c6)sc6c(-n8c9ccccc9c9ccccc98)cccc67)ccc5c34)n2)cc1 ; c1ccc(-c2nc(-c3ccccc3)nc(-c3cccc4sc5cc(-c6ccc7c(c6)sc6cc(-n8c9ccccc9c9ccccc98)ccc67)ccc5c34)n2)cc1 ; c1ccc(-c2nc(-c3ccccc3)nc(-c3cccc4sc5cc(-c6ccc7c(c6)sc6ccc(-n8c9ccccc9c9ccccc98)cc67)ccc5c34)n2)cc1 ; c1ccc(-c2nc(-c3ccccc3)nc(-c3cccc4sc5cc(-c6ccc7c(c6)sc6cccc(-n8c9ccccc9c9ccccc98)c67)ccc5c34)n2)cc1]

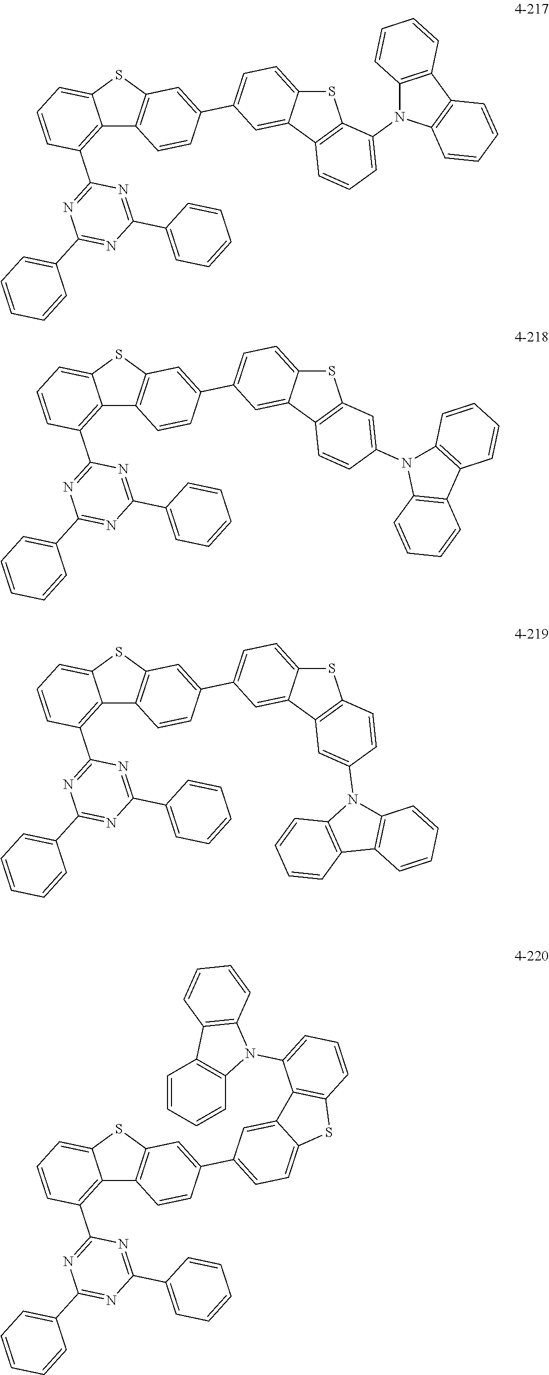 [SMILES: c1ccc(-c2nc(-c3ccccc3)nc(-c3cccc4sc5cc(-c6ccc7sc8c(-n9c%10ccccc%10c%10ccccc%109)cccc8c7c6)ccc5c34)n2)cc1 ; c1ccc(-c2nc(-c3ccccc3)nc(-c3cccc4sc5cc(-c6ccc7sc8cc(-n9c%10ccccc%10c%10ccccc%109)ccc8c7c6)ccc5c34)n2)cc1 ; c1ccc(-c2nc(-c3ccccc3)nc(-c3cccc4sc5cc(-c6ccc7sc8ccc(-n9c%10ccccc%10c%10ccccc%109)cc8c7c6)ccc5c34)n2)cc1 ; c1ccc(-c2nc(-c3ccccc3)nc(-c3cccc4sc5cc(-c6ccc7sc8cccc(-n9c%10ccccc%10c%10ccccc%109)c8c7c6)ccc5c34)n2)cc1]

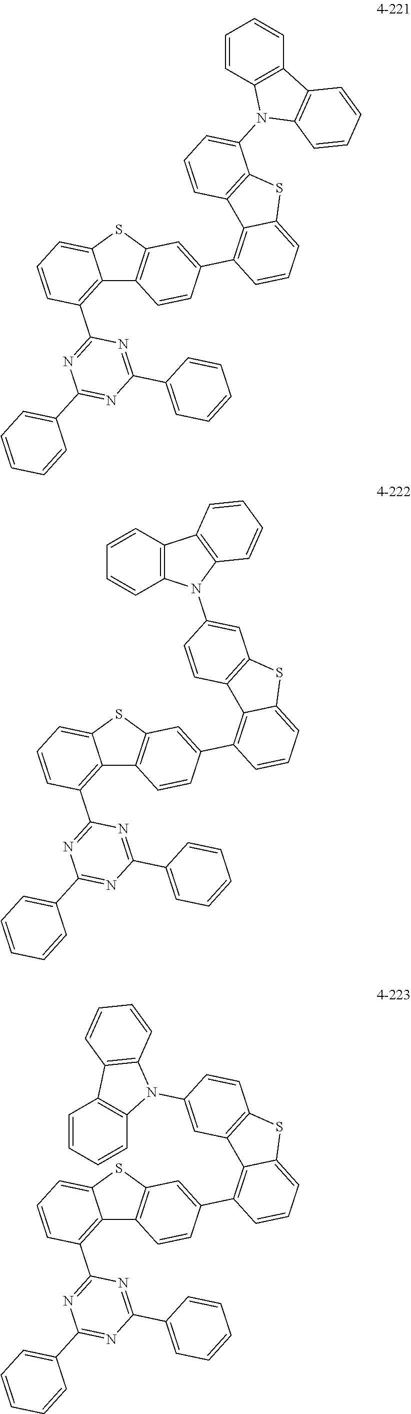 [SMILES: c1ccc(-c2nc(-c3ccccc3)nc(-c3cccc4sc5cc(-c6cccc7sc8c(-n9c%10ccccc%10c%10ccccc%109)cccc8c67)ccc5c34)n2)cc1 ; c1ccc(-c2nc(-c3ccccc3)nc(-c3cccc4sc5cc(-c6cccc7sc8cc(-n9c%10ccccc%10c%10ccccc%109)ccc8c67)ccc5c34)n2)cc1 ; c1ccc(-c2nc(-c3ccccc3)nc(-c3cccc4sc5cc(-c6cccc7sc8ccc(-n9c%10ccccc%10c%10ccccc%109)cc8c67)ccc5c34)n2)cc1]

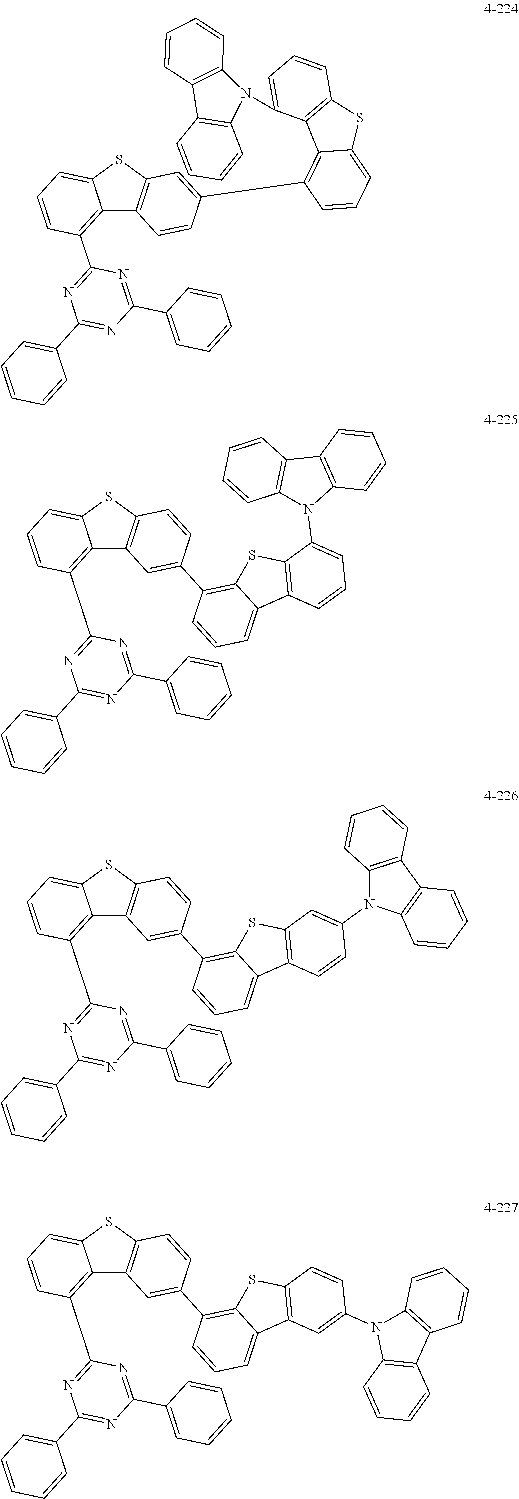 [SMILES: c1ccc(-c2nc(-c3ccccc3)nc(-c3cccc4sc5cc(-c6cccc7sc8cccc(-n9c%10ccccc%10c%10ccccc%109)c8c67)ccc5c34)n2)cc1 ; c1ccc(-c2nc(-c3ccccc3)nc(-c3cccc4sc5ccc(-c6cccc7c6sc6c(-n8c9ccccc9c9ccccc98)cccc67)cc5c34)n2)cc1 ; c1ccc(-c2nc(-c3ccccc3)nc(-c3cccc4sc5ccc(-c6cccc7c6sc6cc(-n8c9ccccc9c9ccccc98)ccc67)cc5c34)n2)cc1 ; c1ccc(-c2nc(-c3ccccc3)nc(-c3cccc4sc5ccc(-c6cccc7c6sc6ccc(-n8c9ccccc9c9ccccc98)cc67)cc5c34)n2)cc1]

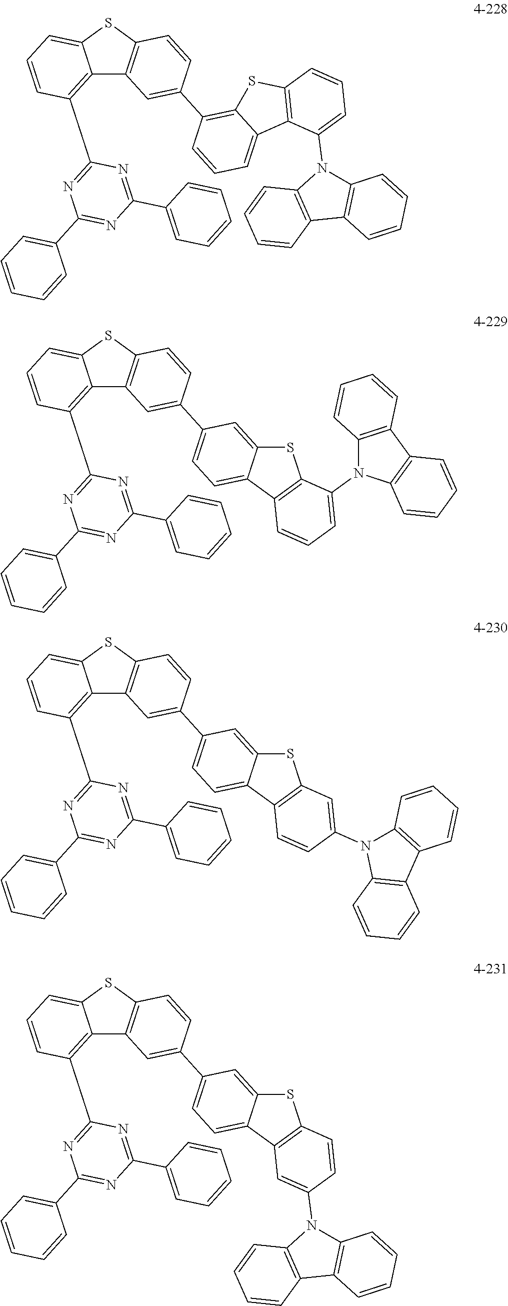

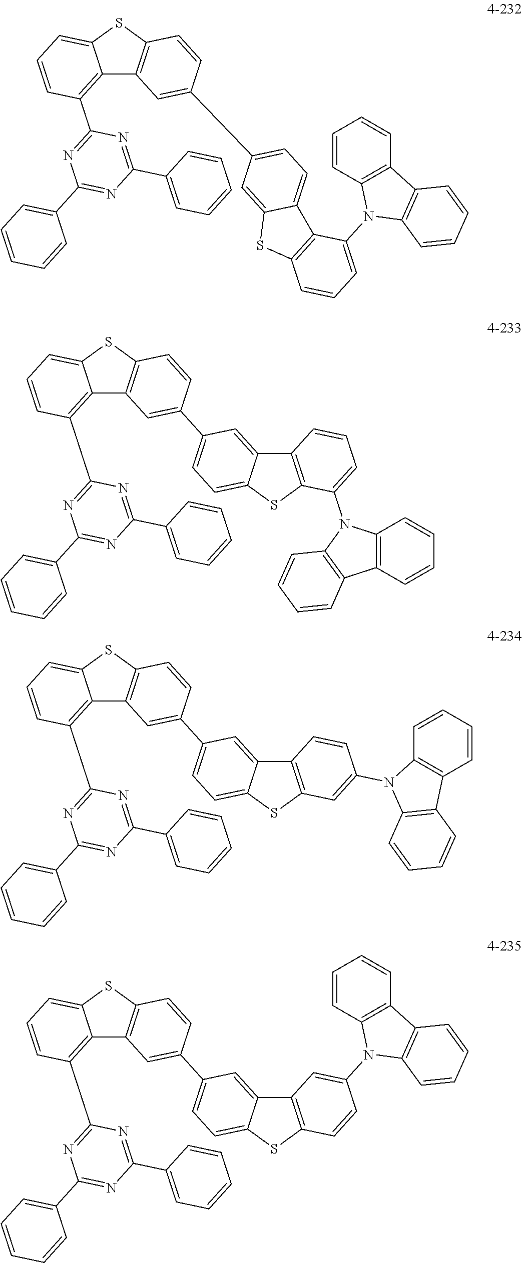

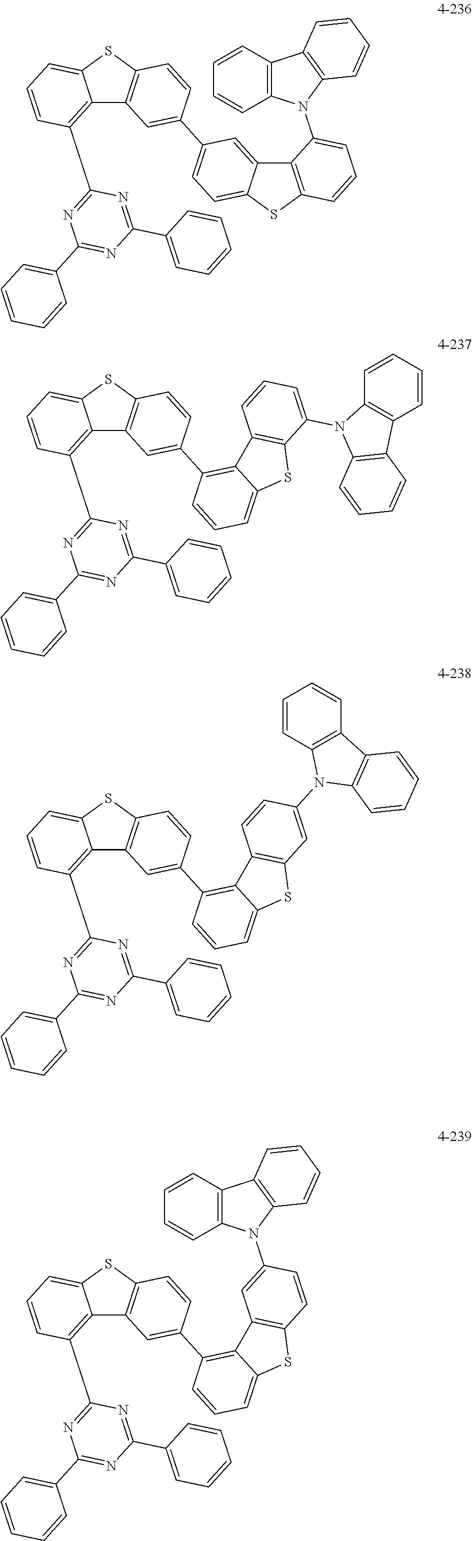 [SMILES: c1ccc(-c2nc(-c3ccccc3)nc(-c3cccc4sc5ccc(-c6ccc7sc8cccc(-n9c%10ccccc%10c%10ccccc%109)c8c7c6)cc5c34)n2)cc1 ; c1ccc(-c2nc(-c3ccccc3)nc(-c3cccc4sc5ccc(-c6cccc7sc8c(-n9c%10ccccc%10c%10ccccc%109)cccc8c67)cc5c34)n2)cc1 ; c1ccc(-c2nc(-c3ccccc3)nc(-c3cccc4sc5ccc(-c6cccc7sc8cc(-n9c%10ccccc%10c%10ccccc%109)ccc8c67)cc5c34)n2)cc1 ; c1ccc(-c2nc(-c3ccccc3)nc(-c3cccc4sc5ccc(-c6cccc7sc8ccc(-n9c%10ccccc%10c%10ccccc%109)cc8c67)cc5c34)n2)cc1]

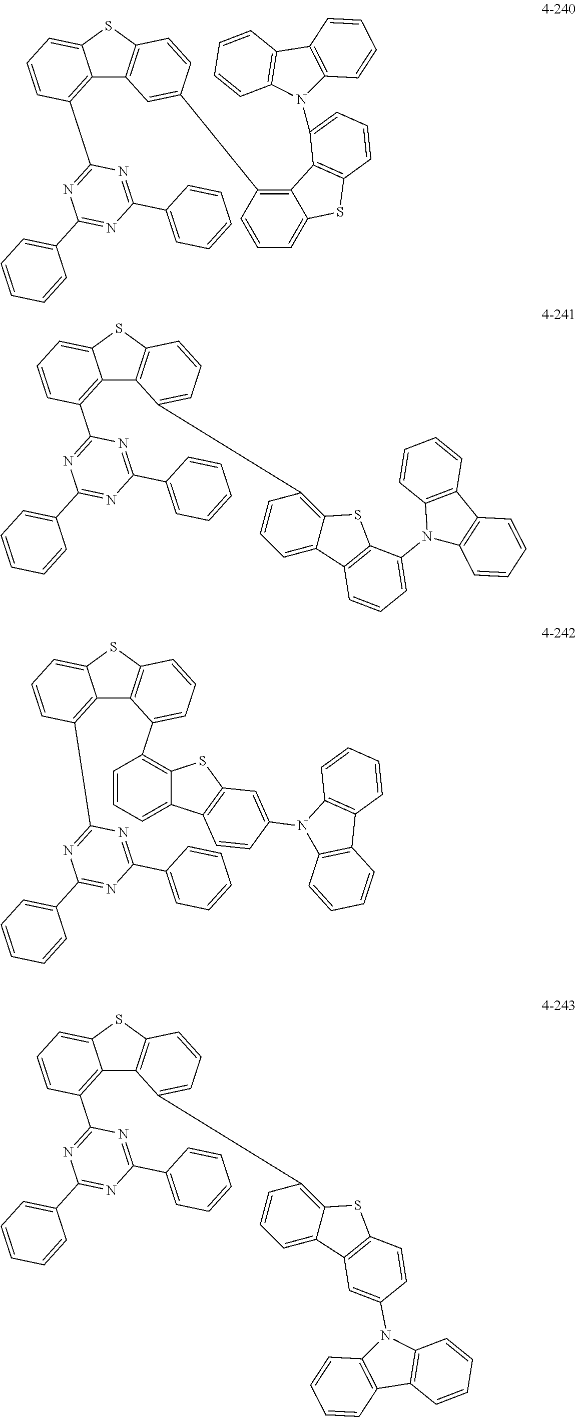

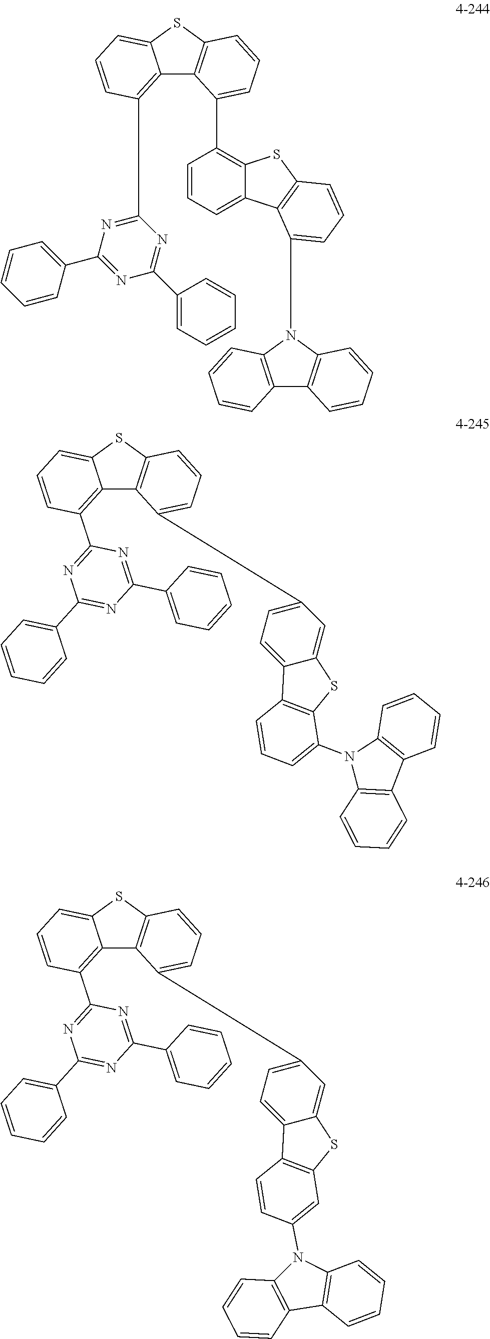 [SMILES: c1ccc(-c2nc(-c3ccccc3)nc(-c3cccc4sc5cccc(-c6cccc7c6sc6cccc(-n8c9ccccc9c9ccccc98)c67)c5c34)n2)cc1 ; c1ccc(-c2nc(-c3ccccc3)nc(-c3cccc4sc5cccc(-c6ccc7c(c6)sc6c(-n8c9ccccc9c9ccccc98)cccc67)c5c34)n2)cc1 ; c1ccc(-c2nc(-c3ccccc3)nc(-c3cccc4sc5cccc(-c6ccc7c(c6)sc6cc(-n8c9ccccc9c9ccccc98)ccc67)c5c34)n2)cc1]

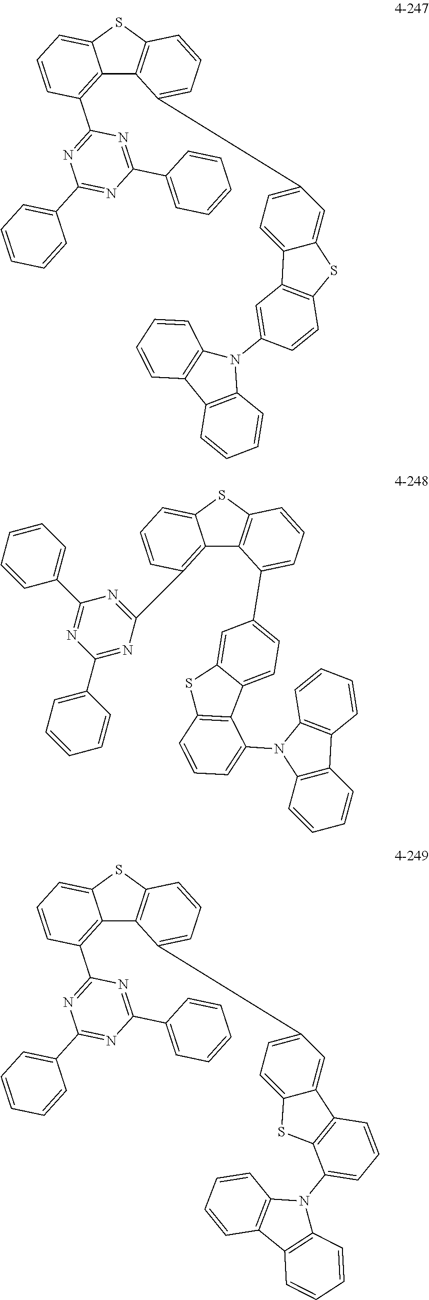

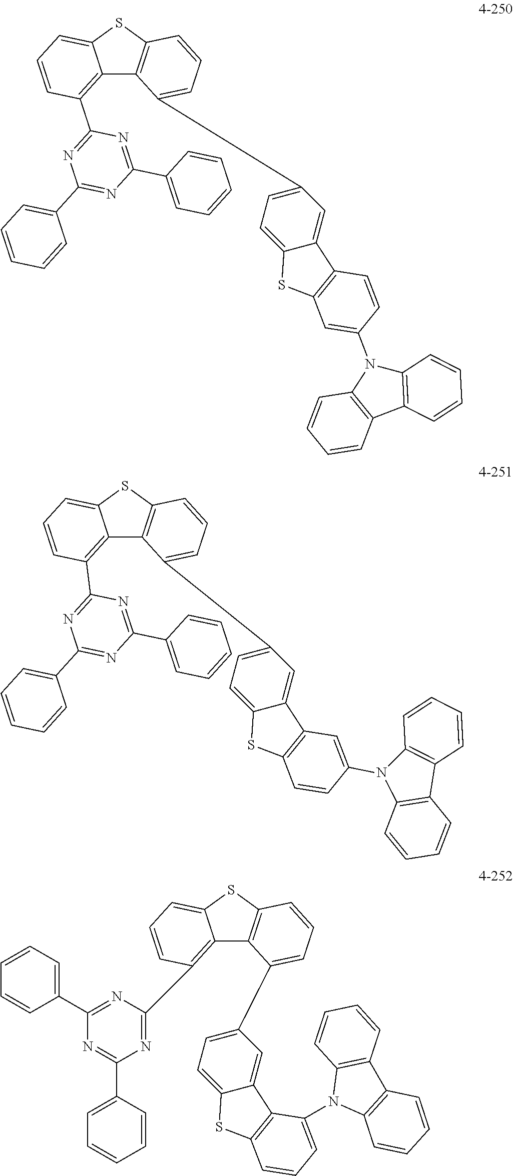 [SMILES: c1ccc(-c2nc(-c3ccccc3)nc(-c3cccc4sc5cccc(-c6ccc7sc8cc(-n9c%10ccccc%10c%10ccccc%109)ccc8c7c6)c5c34)n2)cc1 ; c1ccc(-c2nc(-c3ccccc3)nc(-c3cccc4sc5cccc(-c6ccc7sc8ccc(-n9c%10ccccc%10c%10ccccc%109)cc8c7c6)c5c34)n2)cc1 ; c1ccc(-c2nc(-c3ccccc3)nc(-c3cccc4sc5cccc(-c6ccc7sc8cccc(-n9c%10ccccc%10c%10ccccc%109)c8c7c6)c5c34)n2)cc1]

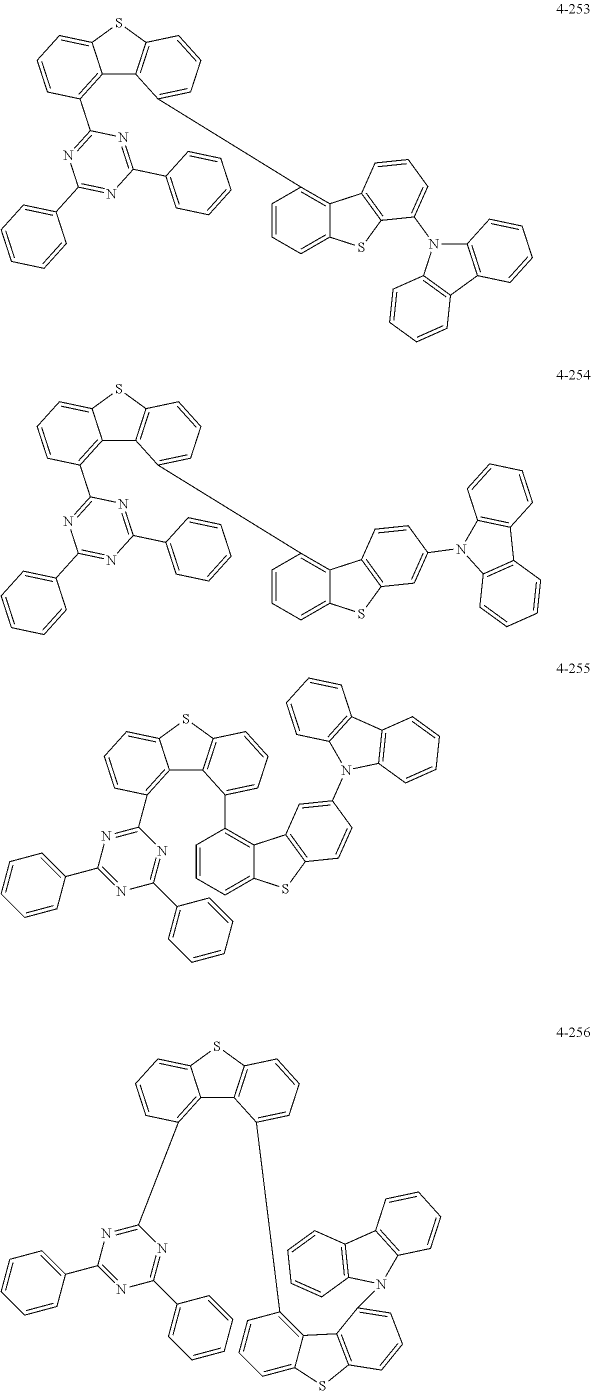 [SMILES: c1ccc(-c2nc(-c3ccccc3)nc(-c3cccc4sc5cccc(-c6cccc7sc8c(-n9c%10ccccc%10c%10ccccc%109)cccc8c67)c5c34)n2)cc1 ; c1ccc(-c2nc(-c3ccccc3)nc(-c3cccc4sc5cccc(-c6cccc7sc8cc(-n9c%10ccccc%10c%10ccccc%109)ccc8c67)c5c34)n2)cc1 ; c1ccc(-c2nc(-c3ccccc3)nc(-c3cccc4sc5cccc(-c6cccc7sc8ccc(-n9c%10ccccc%10c%10ccccc%109)cc8c67)c5c34)n2)cc1 ; c1ccc(-c2nc(-c3ccccc3)nc(-c3cccc4sc5cccc(-c6cccc7sc8cccc(-n9c%10ccccc%10c%10ccccc%109)c8c67)c5c34)n2)cc1]

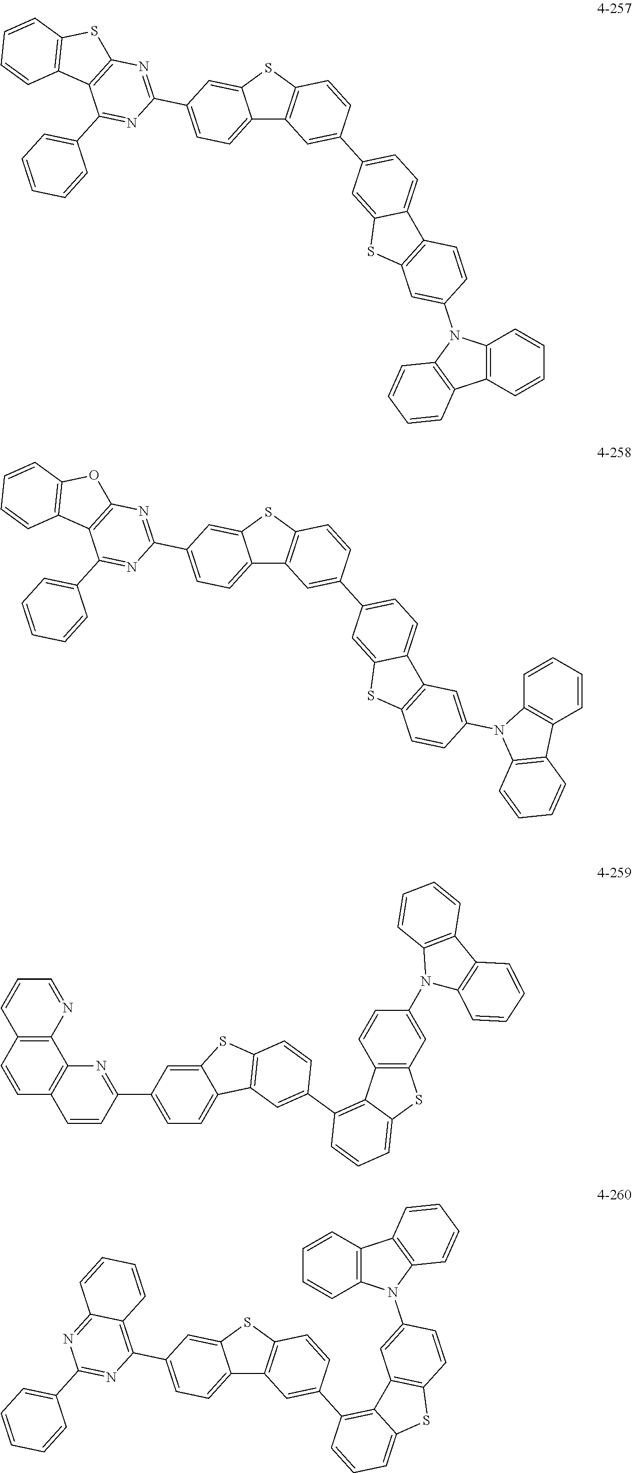 [SMILES: c1ccc(-c2nc(-c3ccc4c(c3)sc3ccc(-c5ccc6c(c5)sc5cc(-n7c8ccccc8c8ccccc87)ccc56)cc34)nc3sc4ccccc4c23)cc1 ; c1ccc(-c2nc(-c3ccc4c(c3)sc3ccc(-c5ccc6c(c5)sc5ccc(-n7c8ccccc8c8ccccc87)cc56)cc34)nc3oc4ccccc4c23)cc1 ; c1cnc2c(c1)ccc1ccc(-c3ccc4c(c3)sc3ccc(-c5cccc6sc7cc(-n8c9ccccc9c9ccccc98)ccc7c56)cc34)nc12 ; c1ccc(-c2nc(-c3ccc4c(c3)sc3ccc(-c5cccc6sc7ccc(-n8c9ccccc9c9ccccc98)cc7c56)cc34)c3ccccc3n2)cc1]

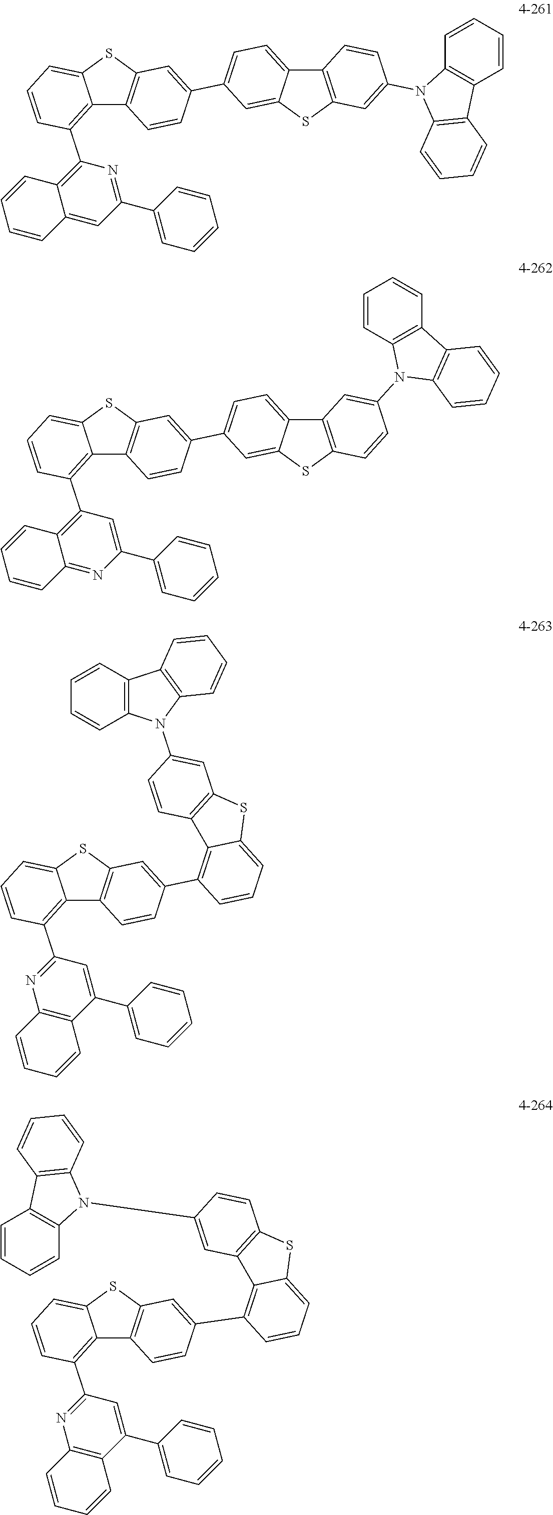

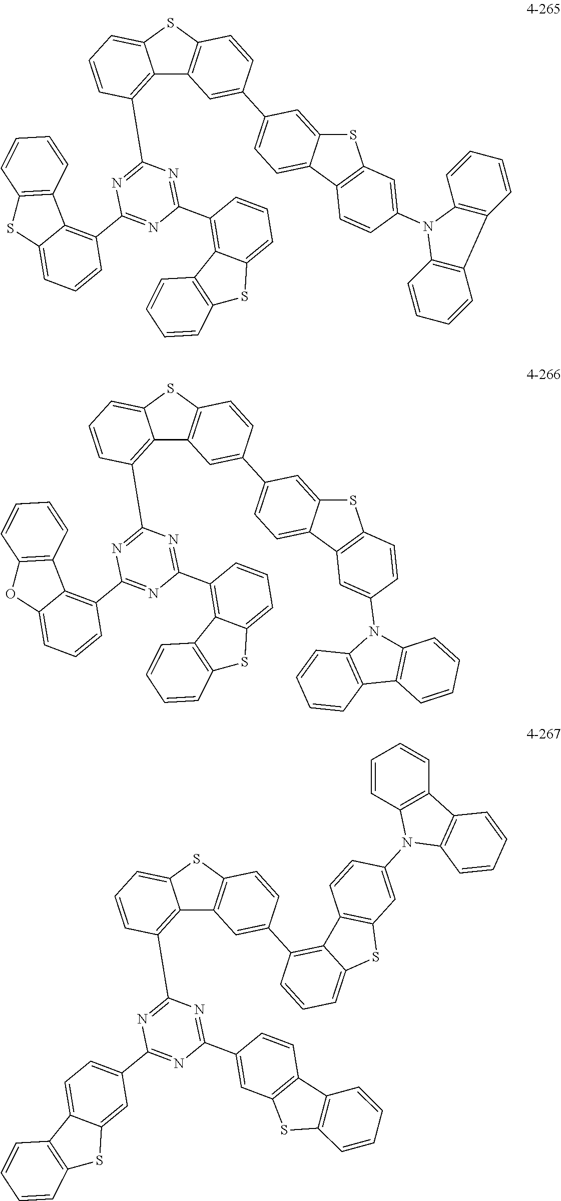

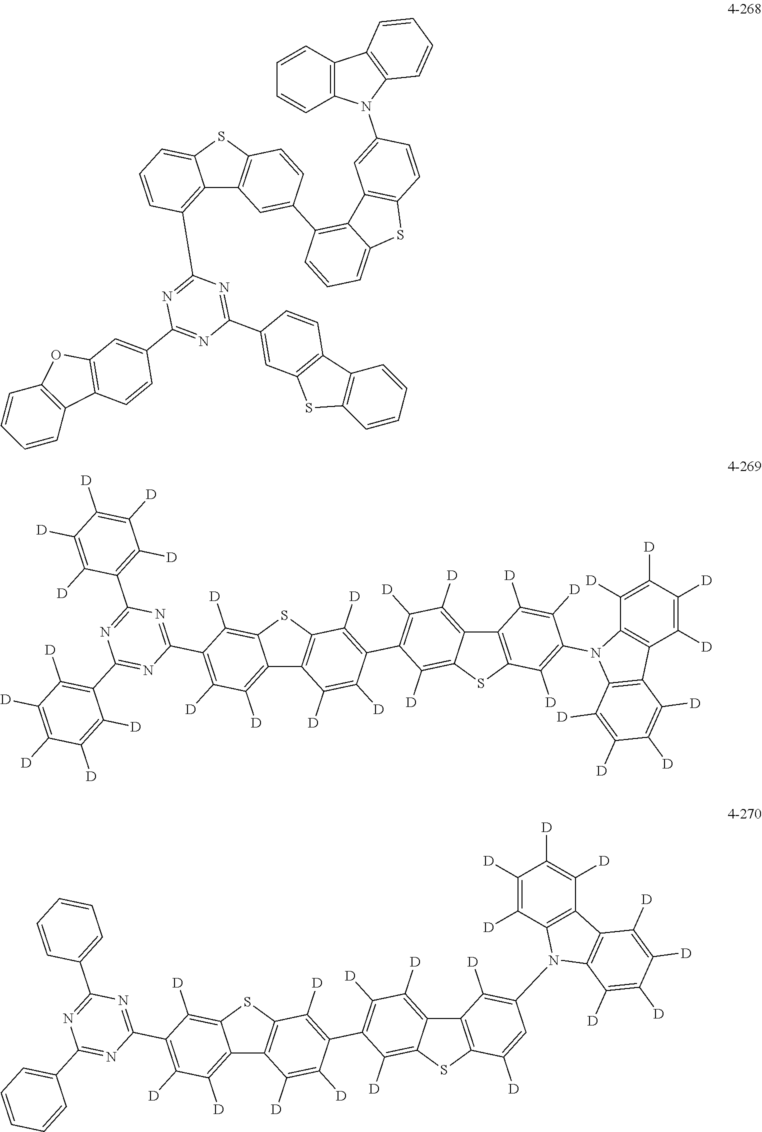

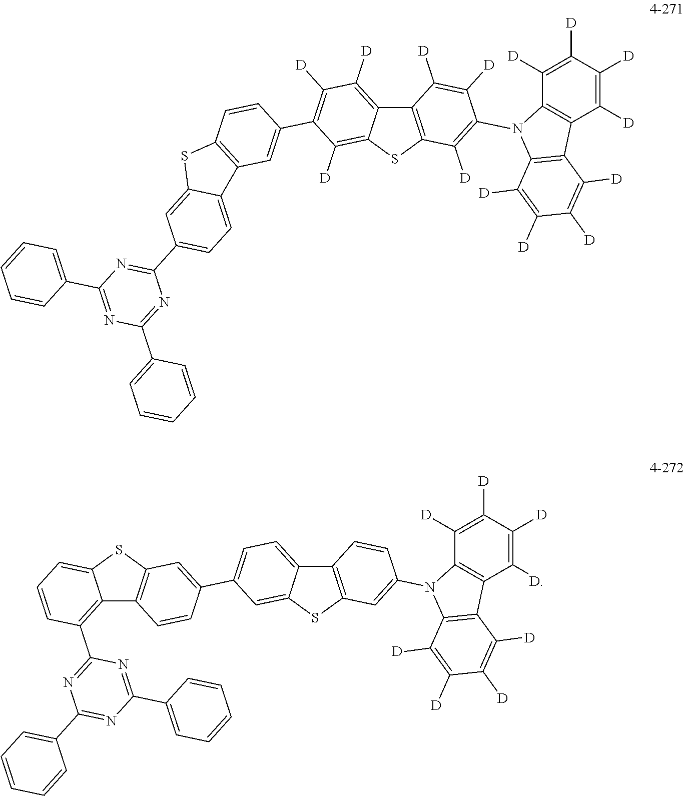

7. An organic light emitting device comprising:
a first electrode;
a second electrode; and
one or more organic material layers provided between the first electrode and the second electrode,
wherein one or more layers of the organic material layers comprise the heterocyclic compound of claim 1.

8. The organic light emitting device of claim 7, wherein the organic material layer comprises one or more light emitting layers, and the light emitting layer comprises the heterocyclic compound.

9. The organic light emitting device of claim 8, wherein the light emitting layer comprises two or more host materials, and at least one of the host materials comprises the heterocyclic compound as a host material of a light emitting material.

10. The organic light emitting device of claim 7, further comprising one, two or more layers selected from the group consisting of a light emitting layer, a hole injection layer, a hole transfer layer, an electron injection layer, an electron transfer layer, a hole auxiliary layer and a hole blocking layer.

11. The organic light emitting device of claim 7, wherein the one or more of the organic material layers comprises the heterocyclic compound as a first compound, and further comprise a heterocyclic compound represented by the following Chemical Formula 13 or 14 as a second compound: 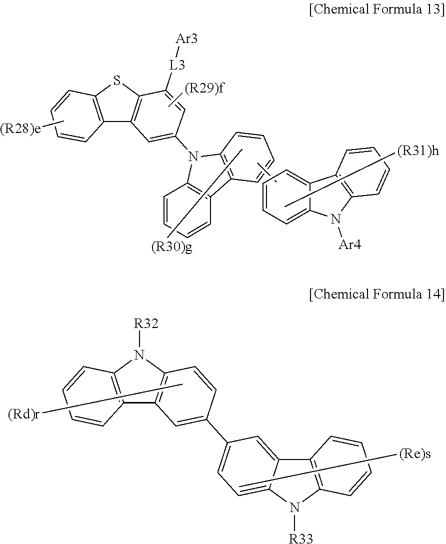
in Chemical Formulae 13 and 14,
L3 is a direct bond; a substituted or unsubstituted arylene group having 6 to 60 carbon atoms; or a substituted or unsubstituted heteroarylene group having 2 to 60 carbon atoms;
R28 to R31 Rd and Re are the same as or different from each other, and each independently selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 2 to 60 carbon atoms; a substituted or unsubstituted cycloakyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more groups adjacent to each other bond to each other to form a substituted or unsubstituted aromatic hydrocarbon ring having 6 to 60 carbon atoms or a substituted or unsubstituted heteroring having 2 to 60 carbon atoms, r and s are an integer of 0 to 7, and when r is 2 or greater, Rds are the same as or different from each other, when s is 2 or greater, Res are the same as or different from each other, e is an integer of 0 to 4, g and h are each an integer of 0 to 7, f is an integer of 0 to 2, and when e is 2 or greater, R28s are the same as or different from each other, when f is an integer of 2, R29s are the same as or different from each other, when g is 2 or greater, R30s are the same as or different from each other, and when h is 2 or greater, R31s are the same as or different from each other;
Ar3 is a substituted or unsubstituted aryl group having 6 to 60 carbon atoms; or a substituted or unsubstituted heteroaryl group having 2 to 60 carbon atoms;
Ar4 is a substituted or unsubstituted aryl group having 6 to 60 carbon atoms; or a substituted or unsubstituted heteroaryl group having 2 to 60 carbon atoms; and
R32 and R33 are the same as or different from each other, and each independently a substituted or unsubstituted silyl group; a substituted or unsubstituted aryl group having 6 to 60 carbon atoms; or a substituted or unsubstituted heteroaryl group having 2 to 60 carbon atoms.

12. The organic light emitting device of claim 11, wherein the heterocyclic compound represented by Chemical Formula 13 has a deuterium content of 0% to 100%.

13. The organic light emitting device of claim 11, wherein the heterocyclic compound represented by Chemical Formula 14 has a deuterium of 0% to 100%.

14. The organic light emitting device of claim 11, wherein Chemical Formula 13 is represented by any one of the following compounds:

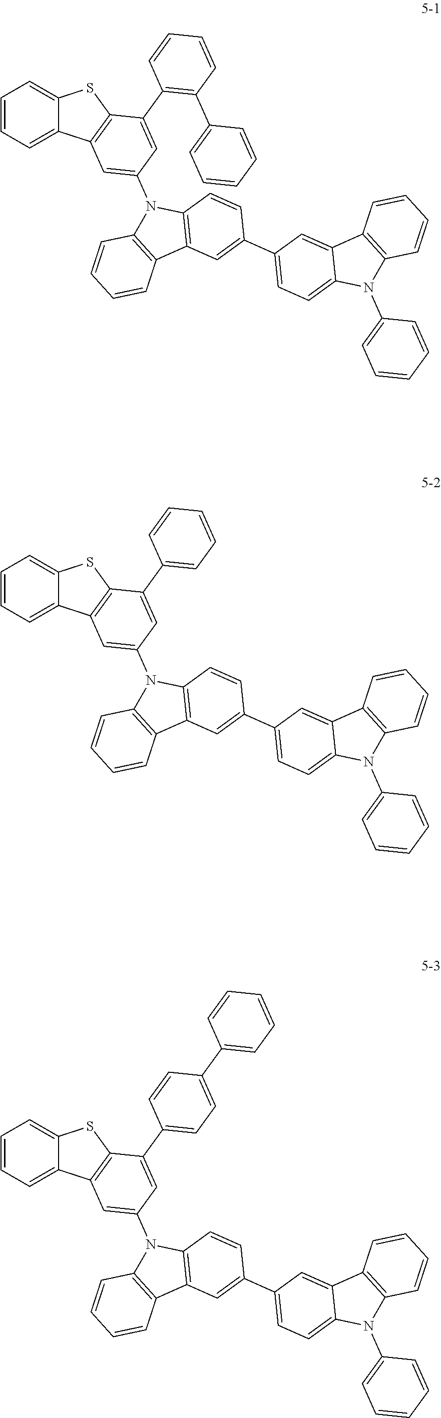 [SMILES: c1ccc(-c2ccccc2-c2cc(-n3c4ccccc4c4cc(-c5ccc6c(c5)c5ccccc5n6-c5ccccc5)ccc43)cc3c2sc2ccccc23)cc1 ; c1ccc(-c2cc(-n3c4ccccc4c4cc(-c5ccc6c(c5)c5ccccc5n6-c5ccccc5)ccc43)cc3c2sc2ccccc23)cc1 ; c1ccc(-c2ccc(-c3cc(-n4c5ccccc5c5cc(-c6ccc7c(c6)c6ccccc6n7-c6ccccc6)ccc54)cc4c3sc3ccccc34)cc2)cc1]

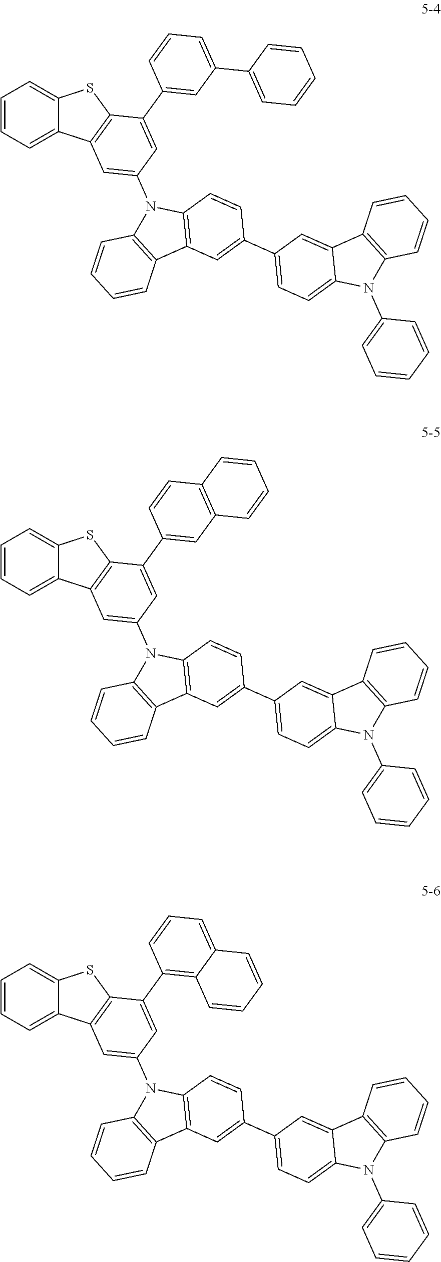 [SMILES: c1ccc(-c2cccc(-c3cc(-n4c5ccccc5c5cc(-c6ccc7c(c6)c6ccccc6n7-c6ccccc6)ccc54)cc4c3sc3ccccc34)c2)cc1 ; c1ccc(-n2c3ccccc3c3cc(-c4ccc5c(c4)c4ccccc4n5-c4cc(-c5ccc6ccccc6c5)c5sc6ccccc6c5c4)ccc32)cc1 ; c1ccc(-n2c3ccccc3c3cc(-c4ccc5c(c4)c4ccccc4n5-c4cc(-c5cccc6ccccc56)c5sc6ccccc6c5c4)ccc32)cc1]

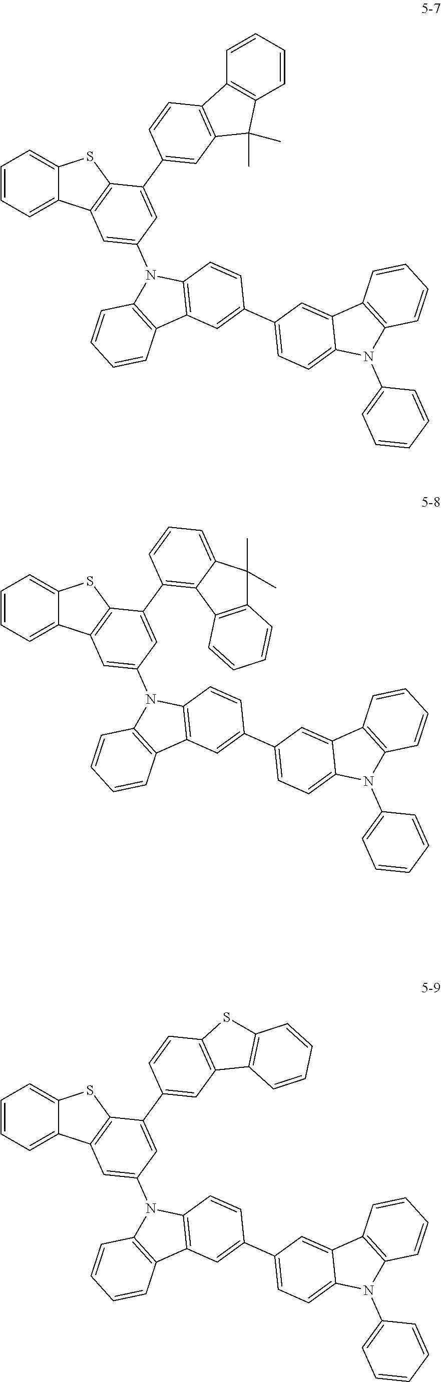 [SMILES: CC1(C)c2ccccc2-c2ccc(-c3cc(-n4c5ccccc5c5cc(-c6ccc7c(c6)c6ccccc6n7-c6ccccc6)ccc54)cc4c3sc3ccccc34)cc21 ; CC1(C)c2ccccc2-c2c(-c3cc(-n4c5ccccc5c5cc(-c6ccc7c(c6)c6ccccc6n7-c6ccccc6)ccc54)cc4c3sc3ccccc34)cccc21 ; c1ccc(-n2c3ccccc3c3cc(-c4ccc5c(c4)c4ccccc4n5-c4cc(-c5ccc6sc7ccccc7c6c5)c5sc6ccccc6c5c4)ccc32)cc1]

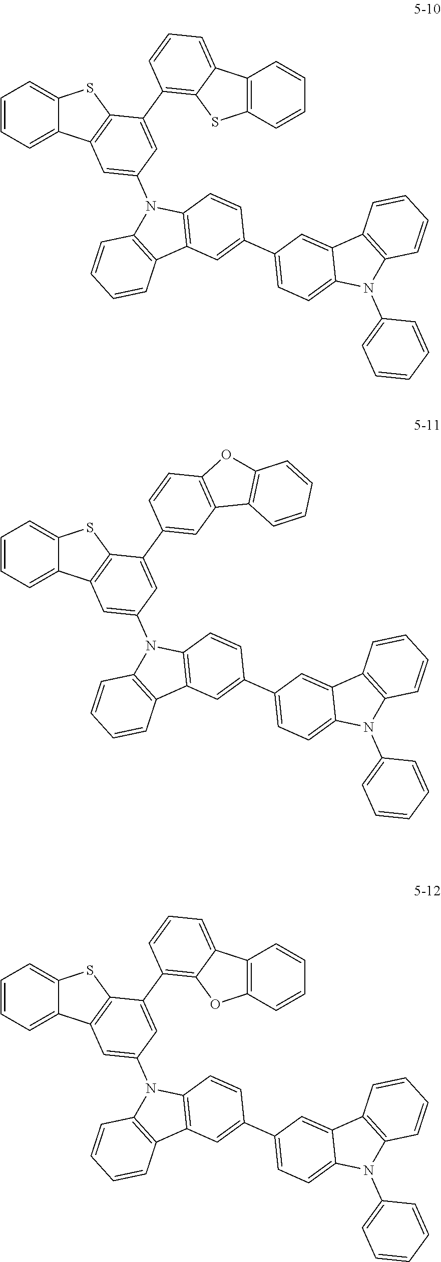 [SMILES: c1ccc(-n2c3ccccc3c3cc(-c4ccc5c(c4)c4ccccc4n5-c4cc(-c5cccc6c5sc5ccccc56)c5sc6ccccc6c5c4)ccc32)cc1 ; c1ccc(-n2c3ccccc3c3cc(-c4ccc5c(c4)c4ccccc4n5-c4cc(-c5ccc6oc7ccccc7c6c5)c5sc6ccccc6c5c4)ccc32)cc1 ; c1ccc(-n2c3ccccc3c3cc(-c4ccc5c(c4)c4ccccc4n5-c4cc(-c5cccc6c5oc5ccccc56)c5sc6ccccc6c5c4)ccc32)cc1]

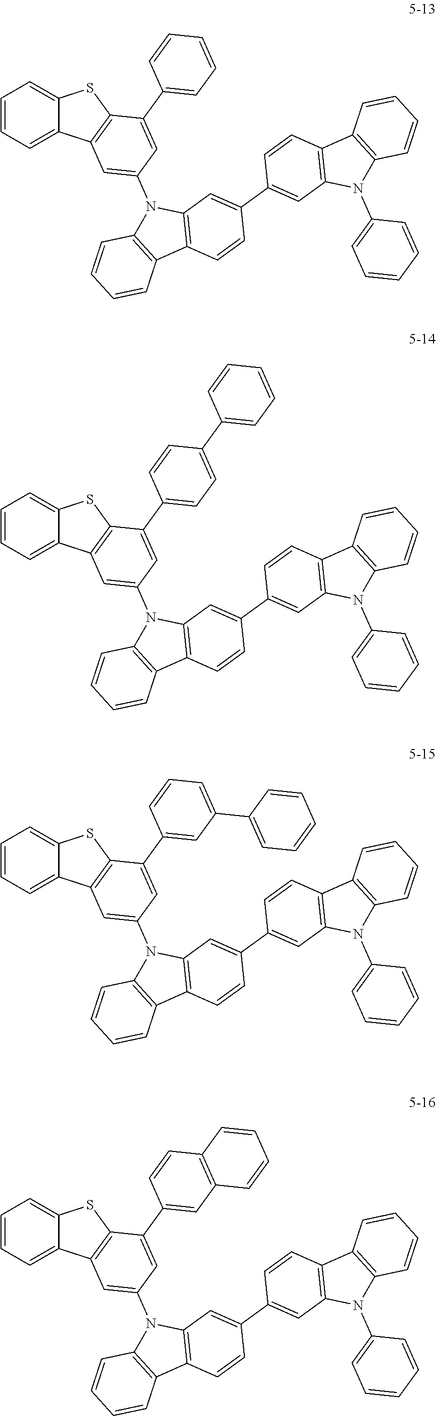 [SMILES: c1ccc(-c2cc(-n3c4ccccc4c4ccc(-c5ccc6c7ccccc7n(-c7ccccc7)c6c5)cc43)cc3c2sc2ccccc23)cc1 ; c1ccc(-c2ccc(-c3cc(-n4c5ccccc5c5ccc(-c6ccc7c8ccccc8n(-c8ccccc8)c7c6)cc54)cc4c3sc3ccccc34)cc2)cc1 ; c1ccc(-c2cccc(-c3cc(-n4c5ccccc5c5ccc(-c6ccc7c8ccccc8n(-c8ccccc8)c7c6)cc54)cc4c3sc3ccccc34)c2)cc1 ; c1ccc(-n2c3ccccc3c3ccc(-c4ccc5c6ccccc6n(-c6cc(-c7ccc8ccccc8c7)c7sc8ccccc8c7c6)c5c4)cc32)cc1]

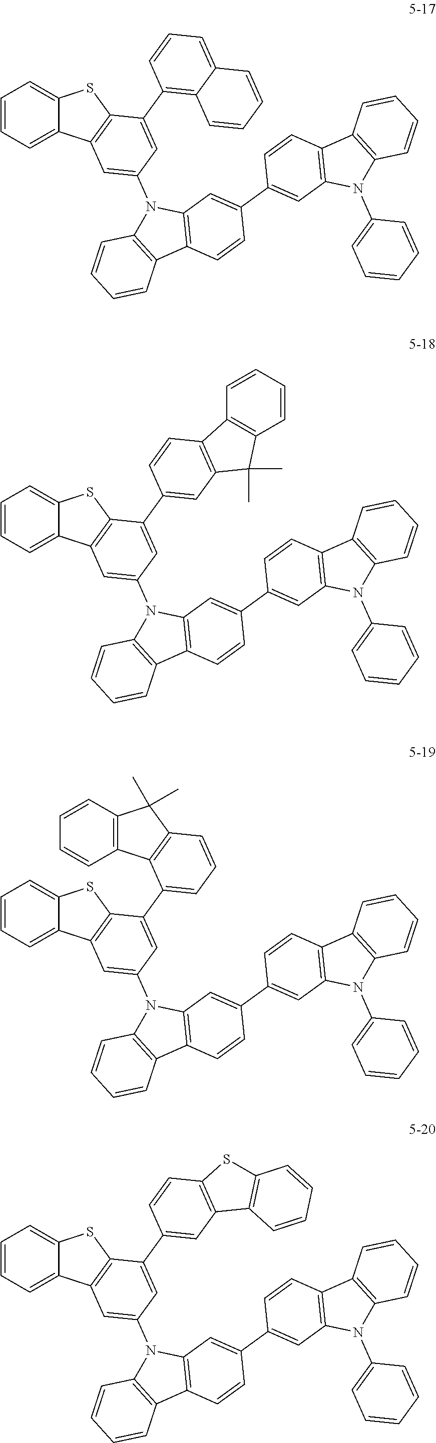 [SMILES: c1ccc(-n2c3ccccc3c3ccc(-c4ccc5c6ccccc6n(-c6cc(-c7cccc8ccccc78)c7sc8ccccc8c7c6)c5c4)cc32)cc1 ; CC1(C)c2ccccc2-c2ccc(-c3cc(-n4c5ccccc5c5ccc(-c6ccc7c8ccccc8n(-c8ccccc8)c7c6)cc54)cc4c3sc3ccccc34)cc21 ; CC1(C)c2ccccc2-c2c(-c3cc(-n4c5ccccc5c5ccc(-c6ccc7c8ccccc8n(-c8ccccc8)c7c6)cc54)cc4c3sc3ccccc34)cccc21 ; c1ccc(-n2c3ccccc3c3ccc(-c4ccc5c6ccccc6n(-c6cc(-c7ccc8sc9ccccc9c8c7)c7sc8ccccc8c7c6)c5c4)cc32)cc1]

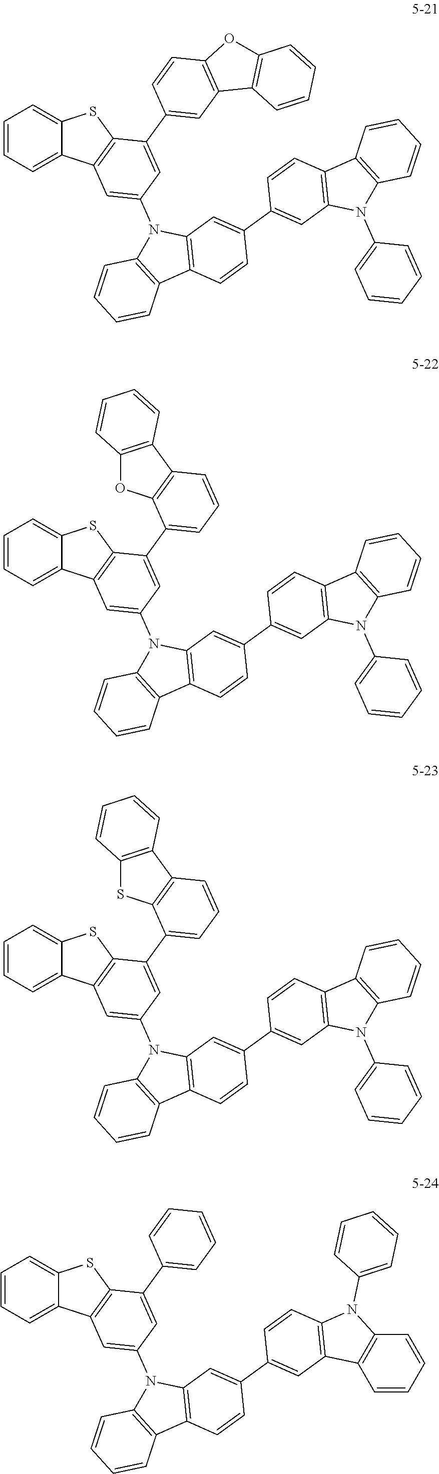 [SMILES: c1ccc(-n2c3ccccc3c3ccc(-c4ccc5c6ccccc6n(-c6cc(-c7ccc8oc9ccccc9c8c7)c7sc8ccccc8c7c6)c5c4)cc32)cc1 ; c1ccc(-n2c3ccccc3c3ccc(-c4ccc5c6ccccc6n(-c6cc(-c7cccc8c7oc7ccccc78)c7sc8ccccc8c7c6)c5c4)cc32)cc1 ; c1ccc(-n2c3ccccc3c3ccc(-c4ccc5c6ccccc6n(-c6cc(-c7cccc8c7sc7ccccc78)c7sc8ccccc8c7c6)c5c4)cc32)cc1 ; c1ccc(-c2cc(-n3c4ccccc4c4ccc(-c5ccc6c(c5)c5ccccc5n6-c5ccccc5)cc43)cc3c2sc2ccccc23)cc1]

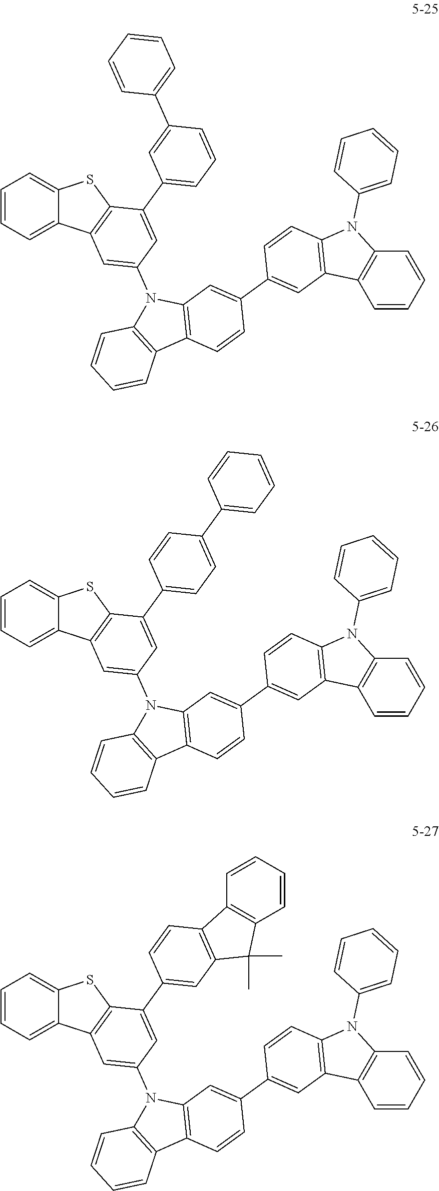 [SMILES: c1ccc(-c2cccc(-c3cc(-n4c5ccccc5c5ccc(-c6ccc7c(c6)c6ccccc6n7-c6ccccc6)cc54)cc4c3sc3ccccc34)c2)cc1 ; c1ccc(-c2ccc(-c3cc(-n4c5ccccc5c5ccc(-c6ccc7c(c6)c6ccccc6n7-c6ccccc6)cc54)cc4c3sc3ccccc34)cc2)cc1 ; CC1(C)c2ccccc2-c2ccc(-c3cc(-n4c5ccccc5c5ccc(-c6ccc7c(c6)c6ccccc6n7-c6ccccc6)cc54)cc4c3sc3ccccc34)cc21]

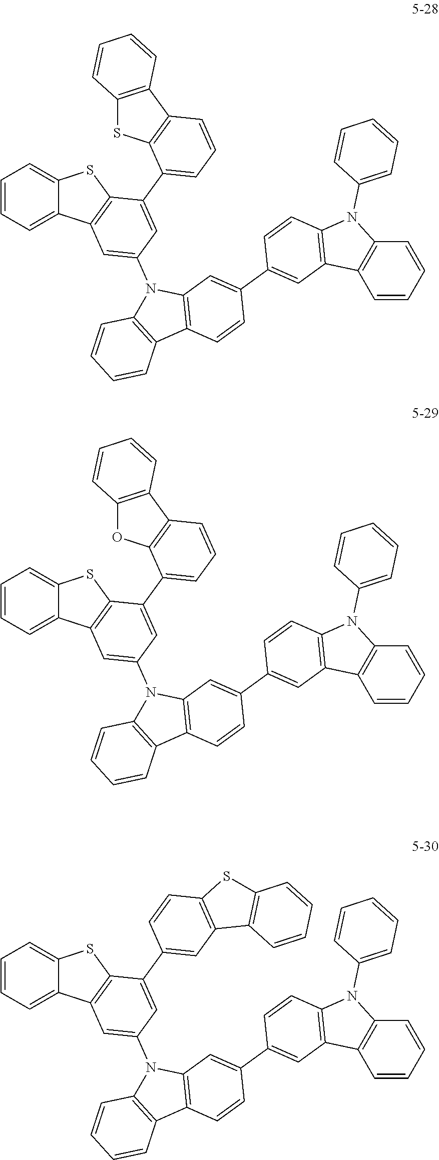 [SMILES: c1ccc(-n2c3ccccc3c3cc(-c4ccc5c6ccccc6n(-c6cc(-c7cccc8c7sc7ccccc78)c7sc8ccccc8c7c6)c5c4)ccc32)cc1 ; c1ccc(-n2c3ccccc3c3cc(-c4ccc5c6ccccc6n(-c6cc(-c7cccc8c7oc7ccccc78)c7sc8ccccc8c7c6)c5c4)ccc32)cc1 ; c1ccc(-n2c3ccccc3c3cc(-c4ccc5c6ccccc6n(-c6cc(-c7ccc8sc9ccccc9c8c7)c7sc8ccccc8c7c6)c5c4)ccc32)cc1]

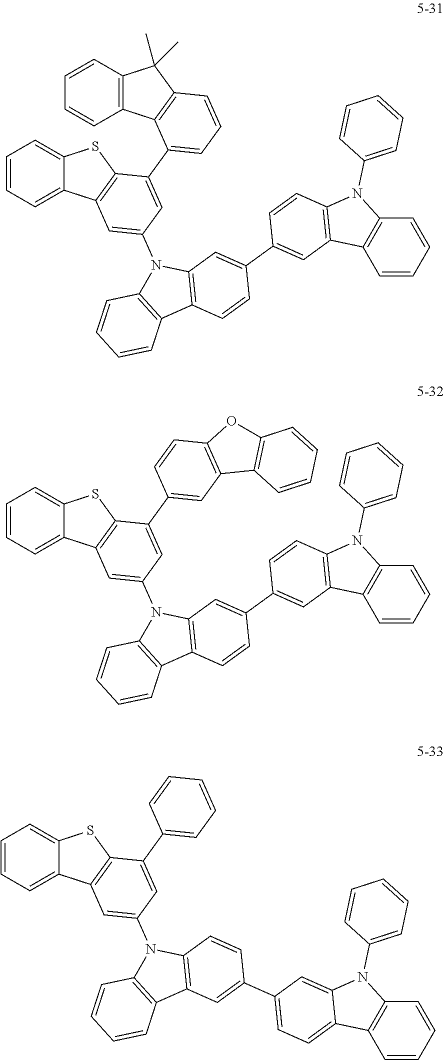 [SMILES: CC1(C)c2ccccc2-c2c(-c3cc(-n4c5ccccc5c5ccc(-c6ccc7c(c6)c6ccccc6n7-c6ccccc6)cc54)cc4c3sc3ccccc34)cccc21 ; c1ccc(-n2c3ccccc3c3cc(-c4ccc5c6ccccc6n(-c6cc(-c7ccc8oc9ccccc9c8c7)c7sc8ccccc8c7c6)c5c4)ccc32)cc1 ; c1ccc(-c2cc(-n3c4ccccc4c4cc(-c5ccc6c7ccccc7n(-c7ccccc7)c6c5)ccc43)cc3c2sc2ccccc23)cc1]

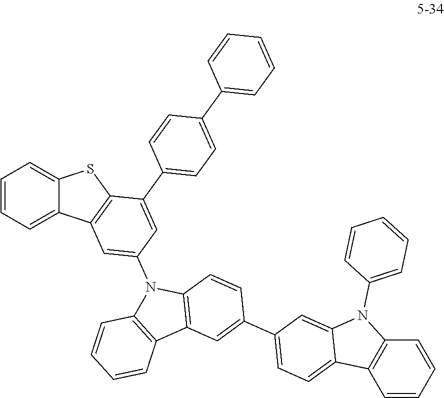 [SMILES: c1ccc(-c2ccc(-c3cc(-n4c5ccccc5c5cc(-c6ccc7c8ccccc8n(-c8ccccc8)c7c6)ccc54)cc4c3sc3ccccc34)cc2)cc1]

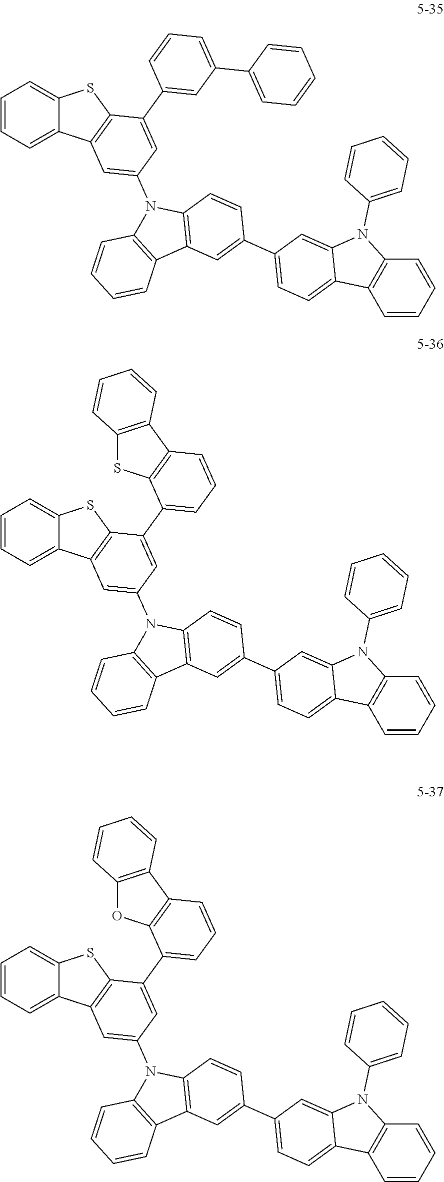 [SMILES: c1ccc(-c2cccc(-c3cc(-n4c5ccccc5c5cc(-c6ccc7c8ccccc8n(-c8ccccc8)c7c6)ccc54)cc4c3sc3ccccc34)c2)cc1 ; c1ccc(-n2c3ccccc3c3ccc(-c4ccc5c(c4)c4ccccc4n5-c4cc(-c5cccc6c5sc5ccccc56)c5sc6ccccc6c5c4)cc32)cc1 ; c1ccc(-n2c3ccccc3c3ccc(-c4ccc5c(c4)c4ccccc4n5-c4cc(-c5cccc6c5oc5ccccc56)c5sc6ccccc6c5c4)cc32)cc1]

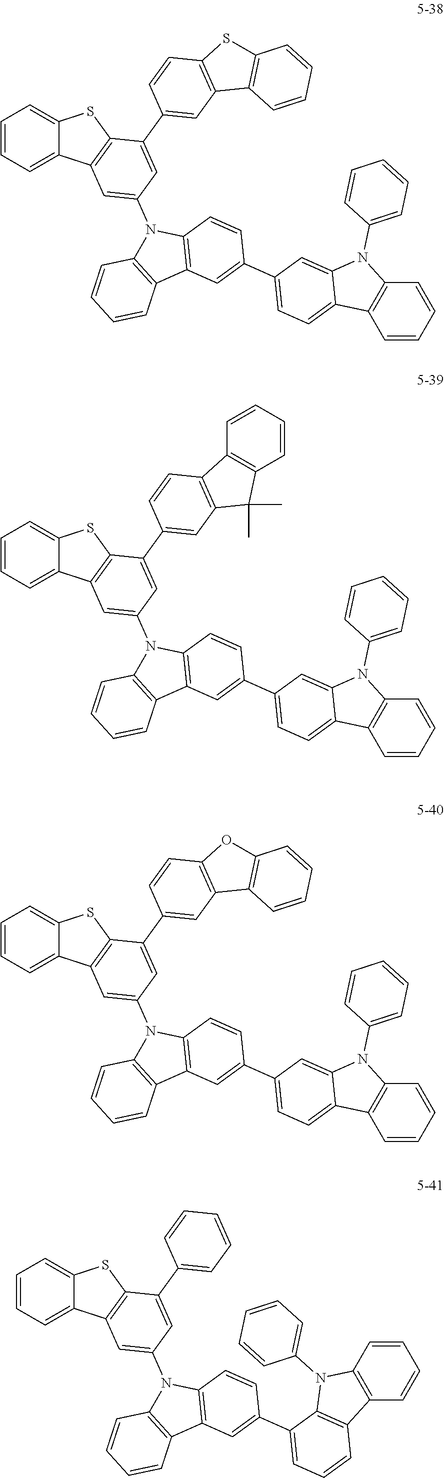 [SMILES: c1ccc(-n2c3ccccc3c3ccc(-c4ccc5c(c4)c4ccccc4n5-c4cc(-c5ccc6sc7ccccc7c6c5)c5sc6ccccc6c5c4)cc32)cc1 ; CC1(C)c2ccccc2-c2ccc(-c3cc(-n4c5ccccc5c5cc(-c6ccc7c8ccccc8n(-c8ccccc8)c7c6)ccc54)cc4c3sc3ccccc34)cc21 ; c1ccc(-n2c3ccccc3c3ccc(-c4ccc5c(c4)c4ccccc4n5-c4cc(-c5ccc6oc7ccccc7c6c5)c5sc6ccccc6c5c4)cc32)cc1 ; c1ccc(-c2cc(-n3c4ccccc4c4cc(-c5cccc6c7ccccc7n(-c7ccccc7)c56)ccc43)cc3c2sc2ccccc23)cc1]

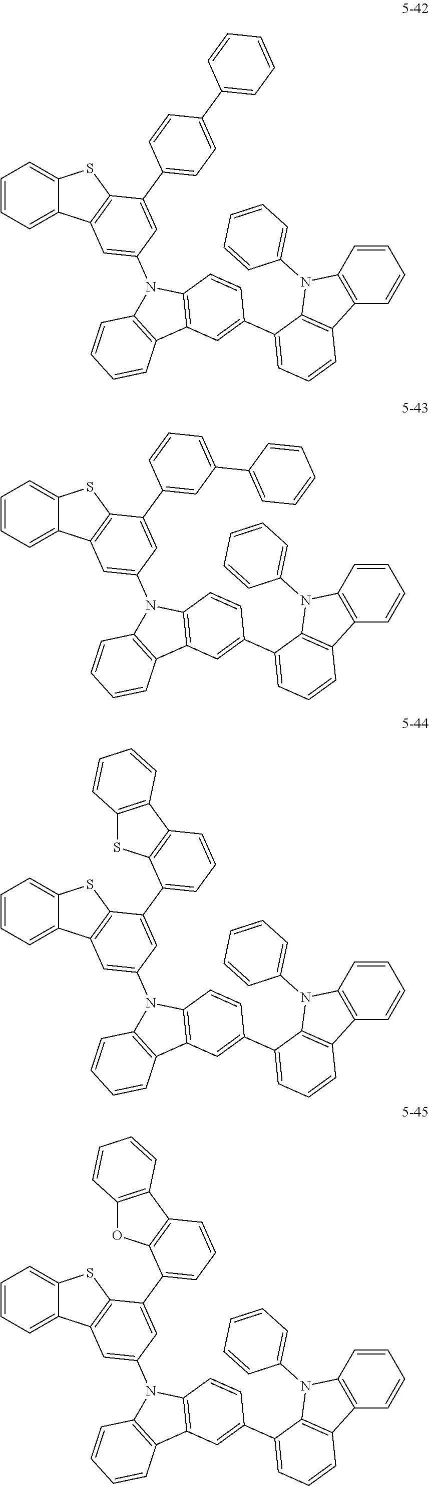

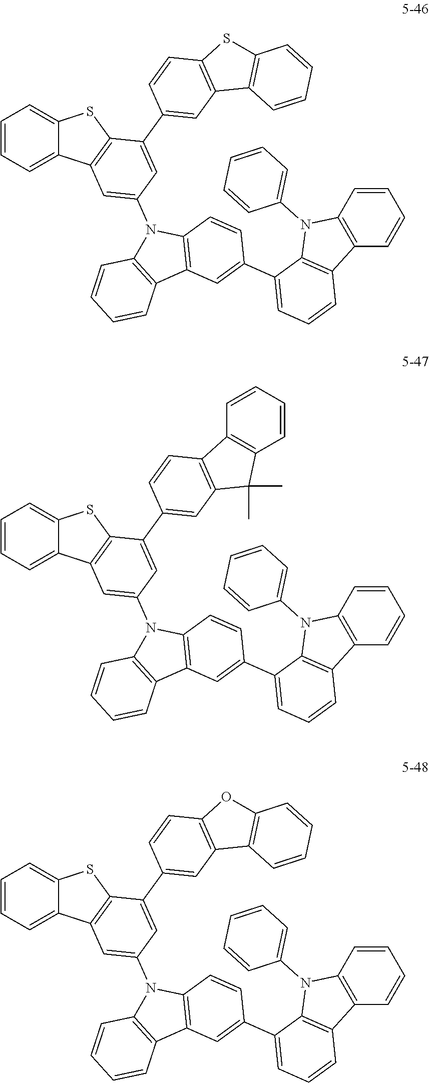 [SMILES: c1ccc(-n2c3ccccc3c3cccc(-c4ccc5c(c4)c4ccccc4n5-c4cc(-c5ccc6sc7ccccc7c6c5)c5sc6ccccc6c5c4)c32)cc1 ; CC1(C)c2ccccc2-c2ccc(-c3cc(-n4c5ccccc5c5cc(-c6cccc7c8ccccc8n(-c8ccccc8)c67)ccc54)cc4c3sc3ccccc34)cc21 ; c1ccc(-n2c3ccccc3c3cccc(-c4ccc5c(c4)c4ccccc4n5-c4cc(-c5ccc6oc7ccccc7c6c5)c5sc6ccccc6c5c4)c32)cc1]

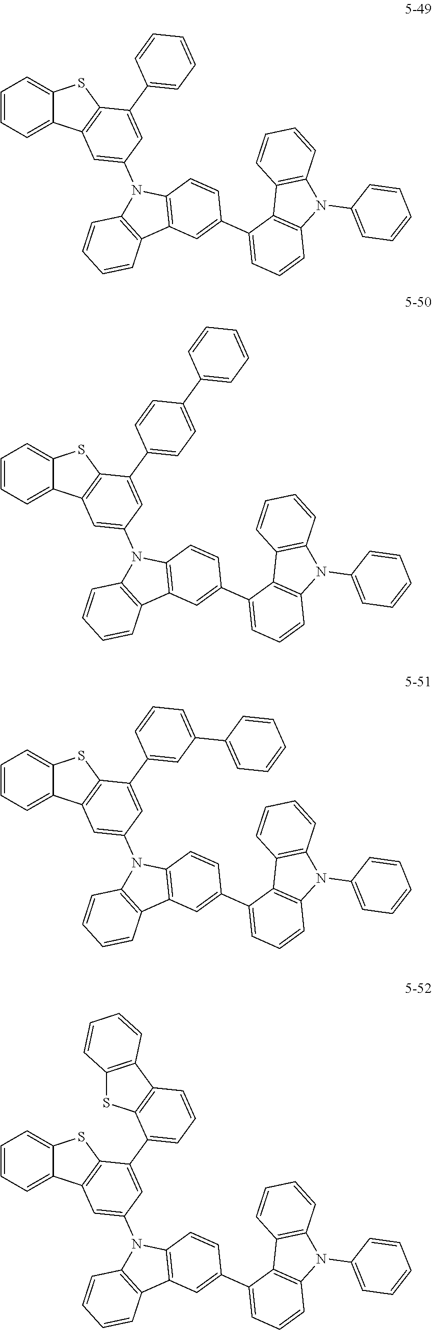 [SMILES: c1ccc(-c2cc(-n3c4ccccc4c4cc(-c5cccc6c5c5ccccc5n6-c5ccccc5)ccc43)cc3c2sc2ccccc23)cc1 ; c1ccc(-c2ccc(-c3cc(-n4c5ccccc5c5cc(-c6cccc7c6c6ccccc6n7-c6ccccc6)ccc54)cc4c3sc3ccccc34)cc2)cc1 ; c1ccc(-c2cccc(-c3cc(-n4c5ccccc5c5cc(-c6cccc7c6c6ccccc6n7-c6ccccc6)ccc54)cc4c3sc3ccccc34)c2)cc1 ; c1ccc(-n2c3ccccc3c3c(-c4ccc5c(c4)c4ccccc4n5-c4cc(-c5cccc6c5sc5ccccc56)c5sc6ccccc6c5c4)cccc32)cc1]

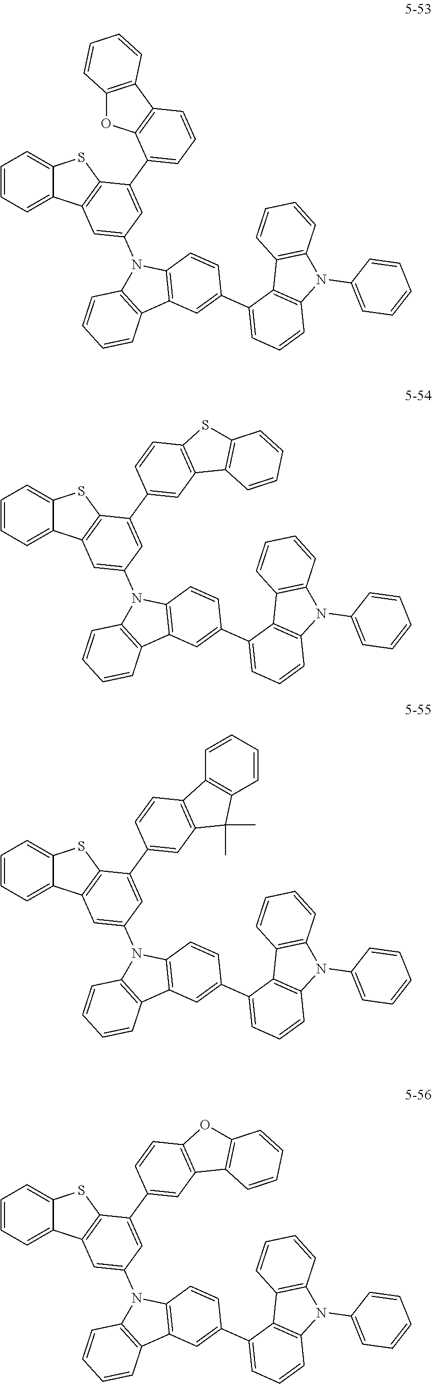 [SMILES: c1ccc(-n2c3ccccc3c3c(-c4ccc5c(c4)c4ccccc4n5-c4cc(-c5cccc6c5oc5ccccc56)c5sc6ccccc6c5c4)cccc32)cc1 ; c1ccc(-n2c3ccccc3c3c(-c4ccc5c(c4)c4ccccc4n5-c4cc(-c5ccc6sc7ccccc7c6c5)c5sc6ccccc6c5c4)cccc32)cc1 ; CC1(C)c2ccccc2-c2ccc(-c3cc(-n4c5ccccc5c5cc(-c6cccc7c6c6ccccc6n7-c6ccccc6)ccc54)cc4c3sc3ccccc34)cc21 ; c1ccc(-n2c3ccccc3c3c(-c4ccc5c(c4)c4ccccc4n5-c4cc(-c5ccc6oc7ccccc7c6c5)c5sc6ccccc6c5c4)cccc32)cc1]

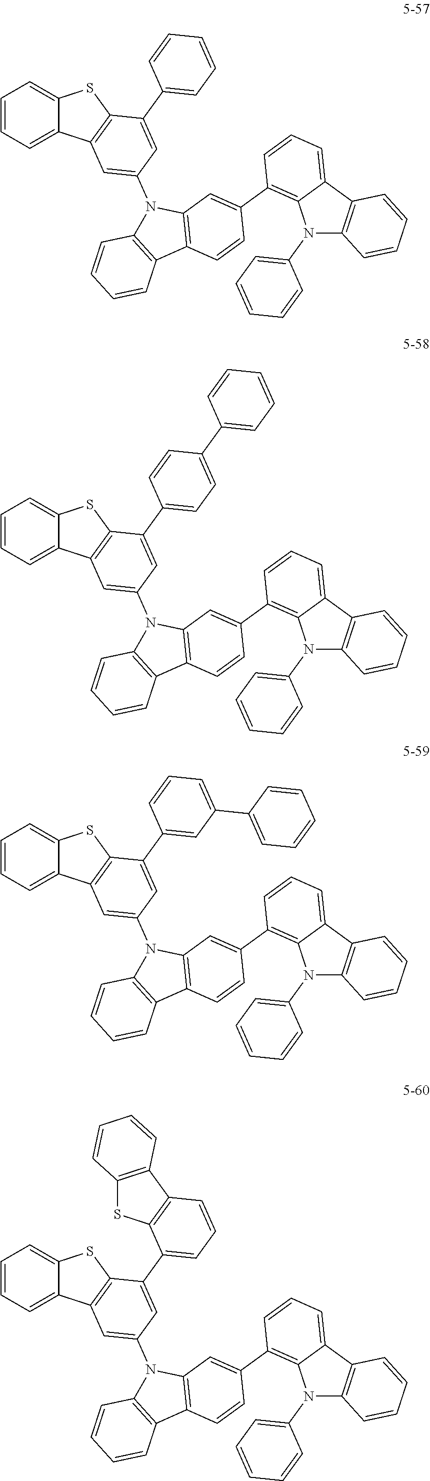 [SMILES: c1ccc(-c2cc(-n3c4ccccc4c4ccc(-c5cccc6c7ccccc7n(-c7ccccc7)c56)cc43)cc3c2sc2ccccc23)cc1 ; c1ccc(-c2ccc(-c3cc(-n4c5ccccc5c5ccc(-c6cccc7c8ccccc8n(-c8ccccc8)c67)cc54)cc4c3sc3ccccc34)cc2)cc1 ; c1ccc(-c2cccc(-c3cc(-n4c5ccccc5c5ccc(-c6cccc7c8ccccc8n(-c8ccccc8)c67)cc54)cc4c3sc3ccccc34)c2)cc1 ; c1ccc(-n2c3ccccc3c3cccc(-c4ccc5c6ccccc6n(-c6cc(-c7cccc8c7sc7ccccc78)c7sc8ccccc8c7c6)c5c4)c32)cc1]

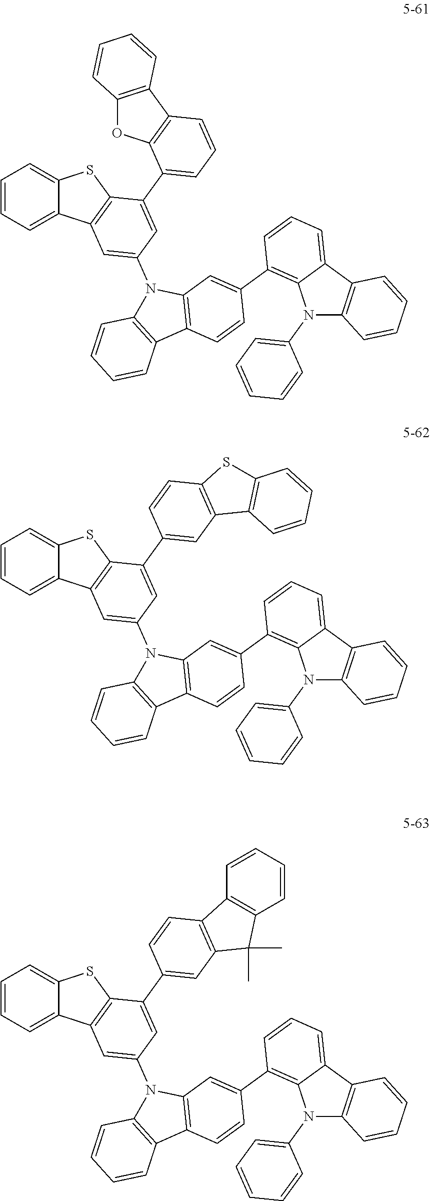 [SMILES: c1ccc(-n2c3ccccc3c3cccc(-c4ccc5c6ccccc6n(-c6cc(-c7cccc8c7oc7ccccc78)c7sc8ccccc8c7c6)c5c4)c32)cc1 ; c1ccc(-n2c3ccccc3c3cccc(-c4ccc5c6ccccc6n(-c6cc(-c7ccc8sc9ccccc9c8c7)c7sc8ccccc8c7c6)c5c4)c32)cc1 ; CC1(C)c2ccccc2-c2ccc(-c3cc(-n4c5ccccc5c5ccc(-c6cccc7c8ccccc8n(-c8ccccc8)c67)cc54)cc4c3sc3ccccc34)cc21]

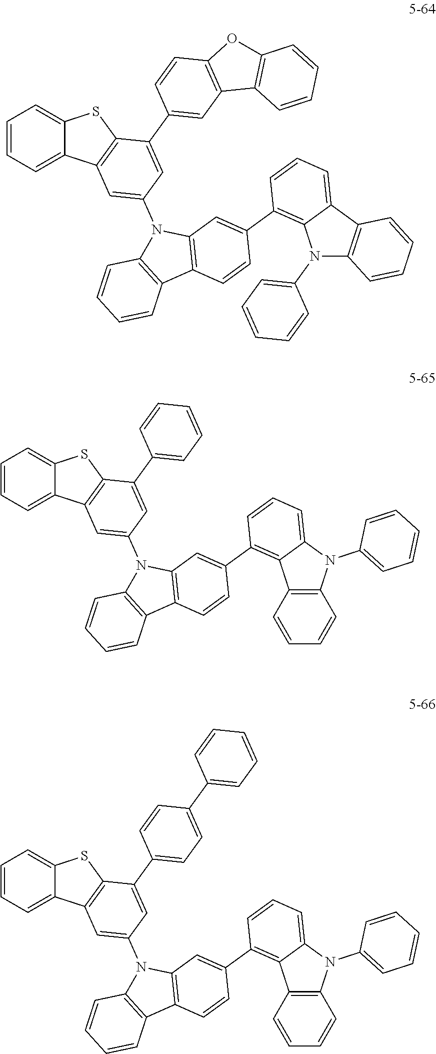 [SMILES: c1ccc(-n2c3ccccc3c3cccc(-c4ccc5c6ccccc6n(-c6cc(-c7ccc8oc9ccccc9c8c7)c7sc8ccccc8c7c6)c5c4)c32)cc1 ; c1ccc(-c2cc(-n3c4ccccc4c4ccc(-c5cccc6c5c5ccccc5n6-c5ccccc5)cc43)cc3c2sc2ccccc23)cc1 ; c1ccc(-c2ccc(-c3cc(-n4c5ccccc5c5ccc(-c6cccc7c6c6ccccc6n7-c6ccccc6)cc54)cc4c3sc3ccccc34)cc2)cc1]

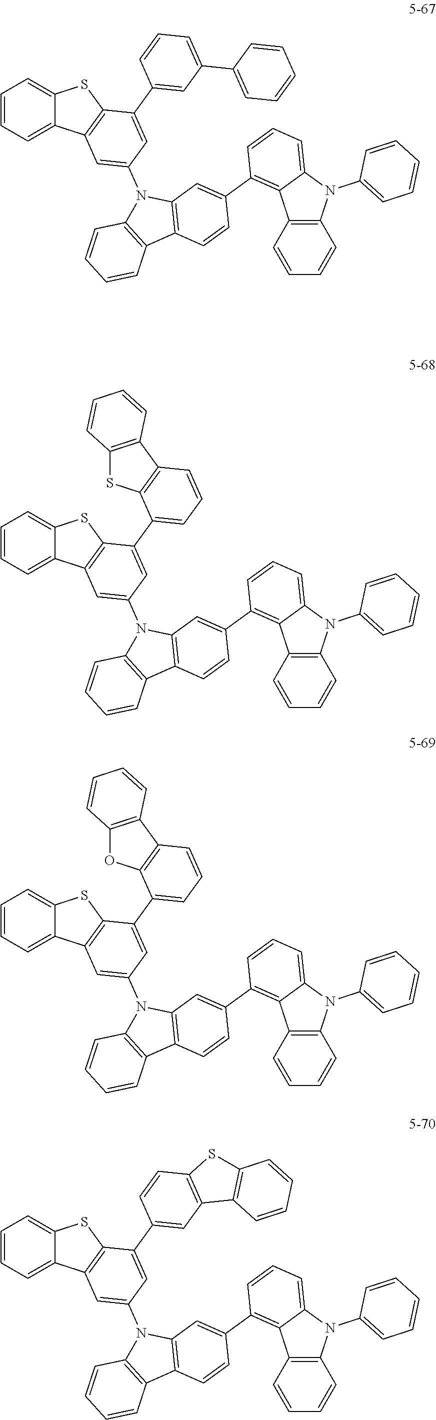 [SMILES: c1ccc(-c2cccc(-c3cc(-n4c5ccccc5c5ccc(-c6cccc7c6c6ccccc6n7-c6ccccc6)cc54)cc4c3sc3ccccc34)c2)cc1 ; c1ccc(-n2c3ccccc3c3c(-c4ccc5c6ccccc6n(-c6cc(-c7cccc8c7sc7ccccc78)c7sc8ccccc8c7c6)c5c4)cccc32)cc1 ; c1ccc(-n2c3ccccc3c3c(-c4ccc5c6ccccc6n(-c6cc(-c7cccc8c7oc7ccccc78)c7sc8ccccc8c7c6)c5c4)cccc32)cc1 ; c1ccc(-n2c3ccccc3c3c(-c4ccc5c6ccccc6n(-c6cc(-c7ccc8sc9ccccc9c8c7)c7sc8ccccc8c7c6)c5c4)cccc32)cc1]

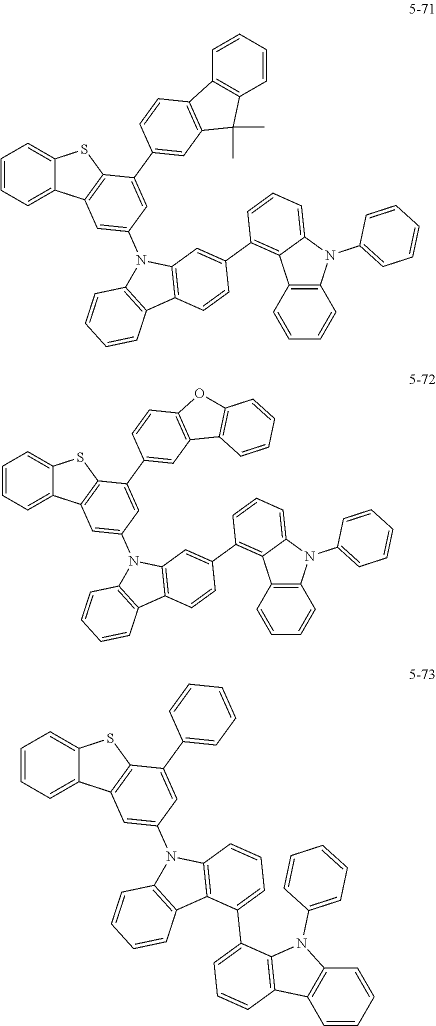 [SMILES: CC1(C)c2ccccc2-c2ccc(-c3cc(-n4c5ccccc5c5ccc(-c6cccc7c6c6ccccc6n7-c6ccccc6)cc54)cc4c3sc3ccccc34)cc21 ; c1ccc(-n2c3ccccc3c3c(-c4ccc5c6ccccc6n(-c6cc(-c7ccc8oc9ccccc9c8c7)c7sc8ccccc8c7c6)c5c4)cccc32)cc1 ; c1ccc(-c2cc(-n3c4ccccc4c4c(-c5cccc6c7ccccc7n(-c7ccccc7)c56)cccc43)cc3c2sc2ccccc23)cc1]

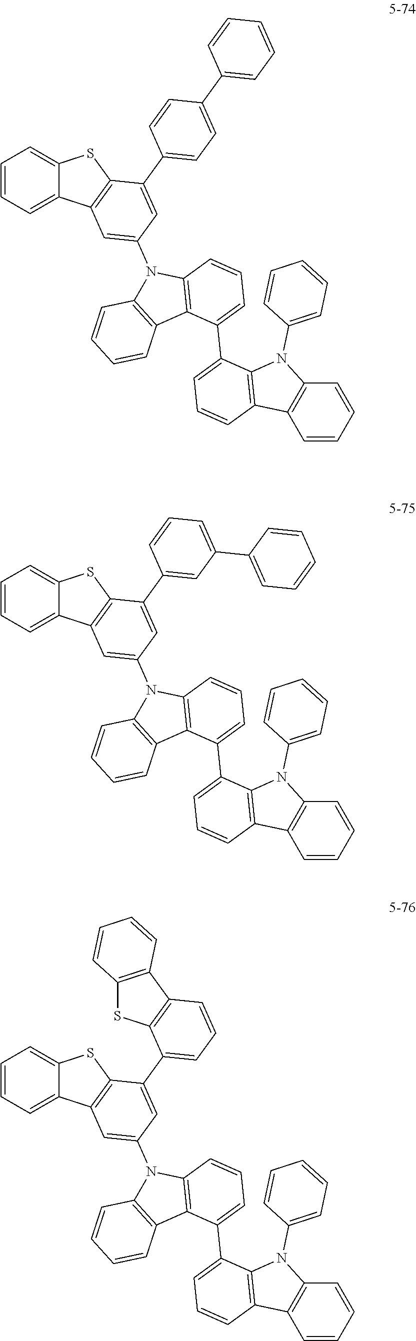 [SMILES: c1ccc(-c2ccc(-c3cc(-n4c5ccccc5c5c(-c6cccc7c8ccccc8n(-c8ccccc8)c67)cccc54)cc4c3sc3ccccc34)cc2)cc1 ; c1ccc(-c2cccc(-c3cc(-n4c5ccccc5c5c(-c6cccc7c8ccccc8n(-c8ccccc8)c67)cccc54)cc4c3sc3ccccc34)c2)cc1 ; c1ccc(-n2c3ccccc3c3cccc(-c4cccc5c4c4ccccc4n5-c4cc(-c5cccc6c5sc5ccccc56)c5sc6ccccc6c5c4)c32)cc1]

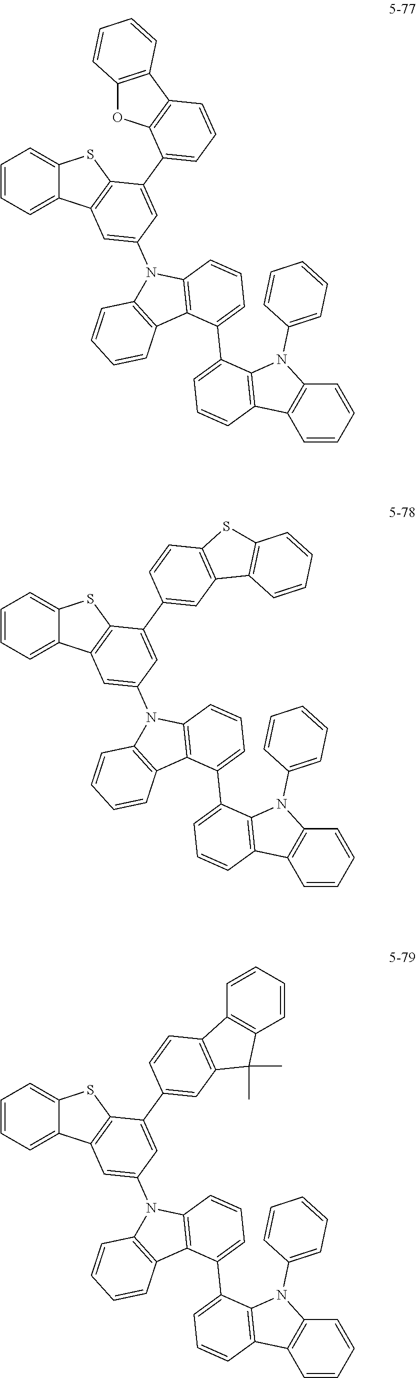 [SMILES: c1ccc(-n2c3ccccc3c3cccc(-c4cccc5c4c4ccccc4n5-c4cc(-c5cccc6c5oc5ccccc56)c5sc6ccccc6c5c4)c32)cc1 ; c1ccc(-n2c3ccccc3c3cccc(-c4cccc5c4c4ccccc4n5-c4cc(-c5ccc6sc7ccccc7c6c5)c5sc6ccccc6c5c4)c32)cc1 ; CC1(C)c2ccccc2-c2ccc(-c3cc(-n4c5ccccc5c5c(-c6cccc7c8ccccc8n(-c8ccccc8)c67)cccc54)cc4c3sc3ccccc34)cc21]

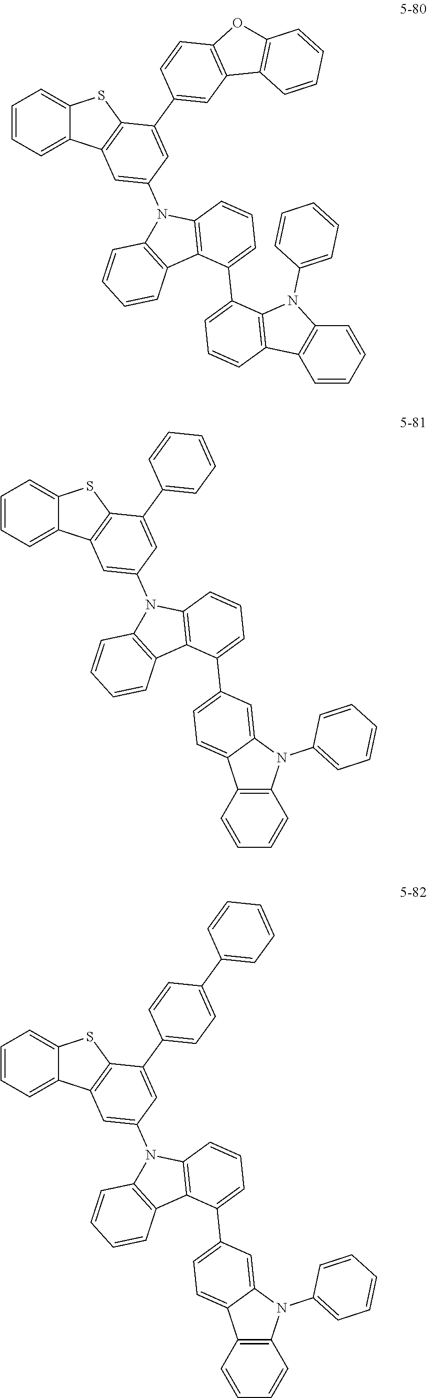 [SMILES: c1ccc(-n2c3ccccc3c3cccc(-c4cccc5c4c4ccccc4n5-c4cc(-c5ccc6oc7ccccc7c6c5)c5sc6ccccc6c5c4)c32)cc1 ; c1ccc(-c2cc(-n3c4ccccc4c4c(-c5ccc6c7ccccc7n(-c7ccccc7)c6c5)cccc43)cc3c2sc2ccccc23)cc1 ; c1ccc(-c2ccc(-c3cc(-n4c5ccccc5c5c(-c6ccc7c8ccccc8n(-c8ccccc8)c7c6)cccc54)cc4c3sc3ccccc34)cc2)cc1]

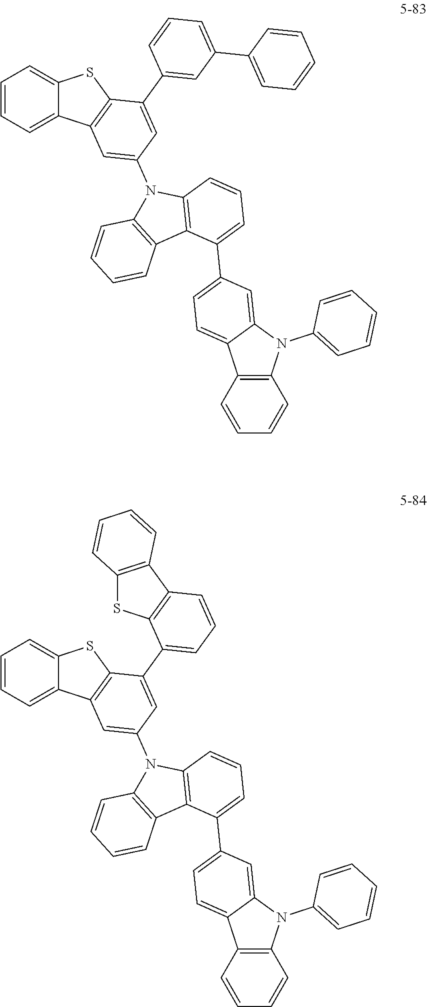 [SMILES: c1ccc(-c2cccc(-c3cc(-n4c5ccccc5c5c(-c6ccc7c8ccccc8n(-c8ccccc8)c7c6)cccc54)cc4c3sc3ccccc34)c2)cc1 ; c1ccc(-n2c3ccccc3c3ccc(-c4cccc5c4c4ccccc4n5-c4cc(-c5cccc6c5sc5ccccc56)c5sc6ccccc6c5c4)cc32)cc1]

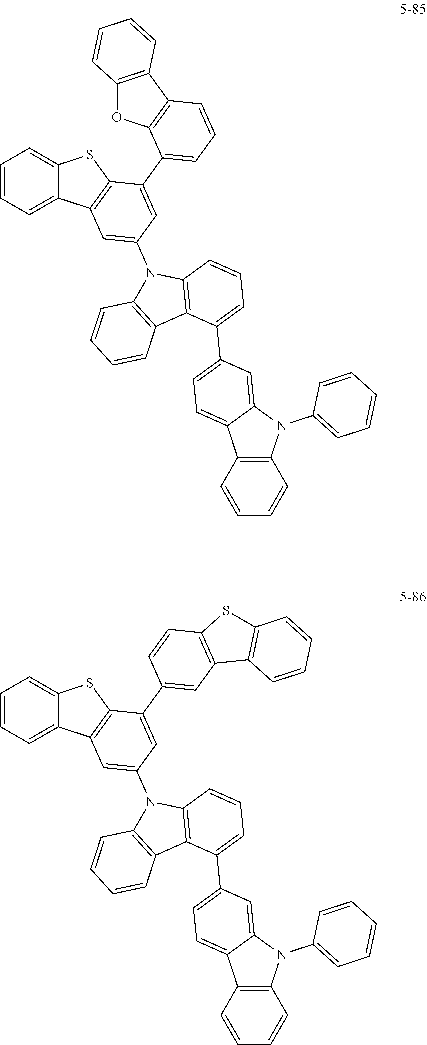 [SMILES: c1ccc(-n2c3ccccc3c3ccc(-c4cccc5c4c4ccccc4n5-c4cc(-c5cccc6c5oc5ccccc56)c5sc6ccccc6c5c4)cc32)cc1 ; c1ccc(-n2c3ccccc3c3ccc(-c4cccc5c4c4ccccc4n5-c4cc(-c5ccc6sc7ccccc7c6c5)c5sc6ccccc6c5c4)cc32)cc1]

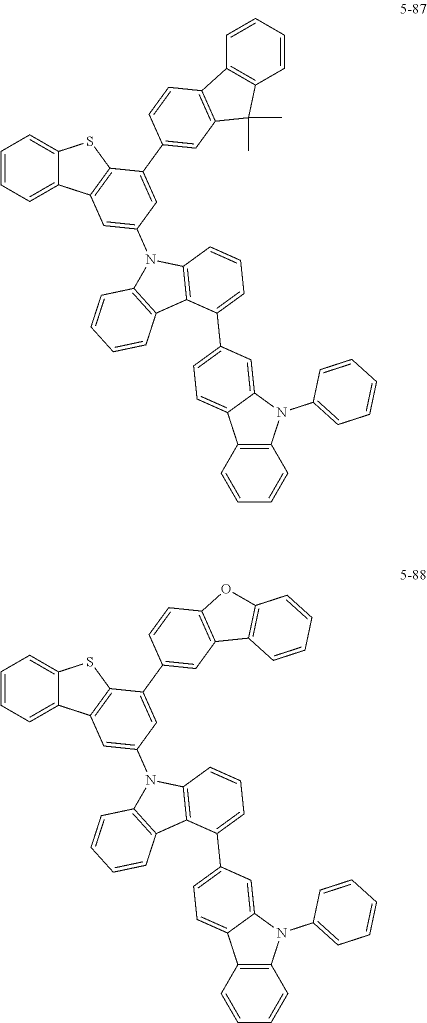 [SMILES: CC1(C)c2ccccc2-c2ccc(-c3cc(-n4c5ccccc5c5c(-c6ccc7c8ccccc8n(-c8ccccc8)c7c6)cccc54)cc4c3sc3ccccc34)cc21 ; c1ccc(-n2c3ccccc3c3ccc(-c4cccc5c4c4ccccc4n5-c4cc(-c5ccc6oc7ccccc7c6c5)c5sc6ccccc6c5c4)cc32)cc1]

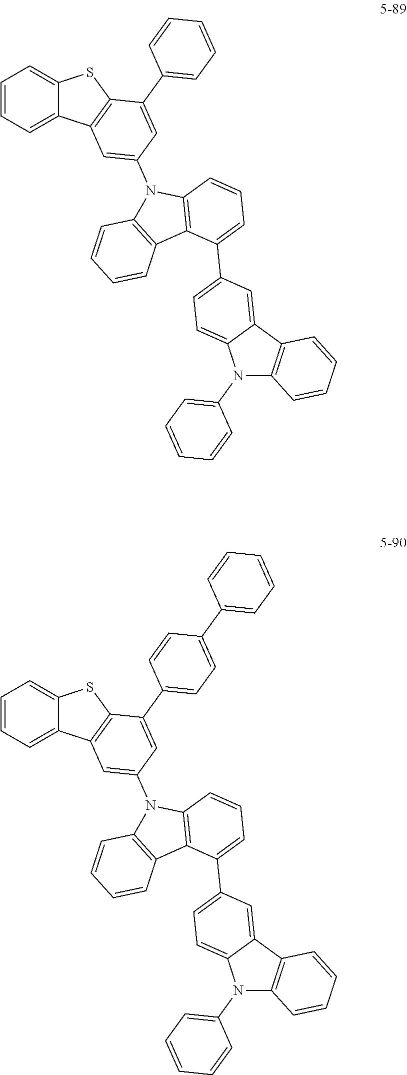 [SMILES: c1ccc(-c2cc(-n3c4ccccc4c4c(-c5ccc6c(c5)c5ccccc5n6-c5ccccc5)cccc43)cc3c2sc2ccccc23)cc1 ; c1ccc(-c2ccc(-c3cc(-n4c5ccccc5c5c(-c6ccc7c(c6)c6ccccc6n7-c6ccccc6)cccc54)cc4c3sc3ccccc34)cc2)cc1]

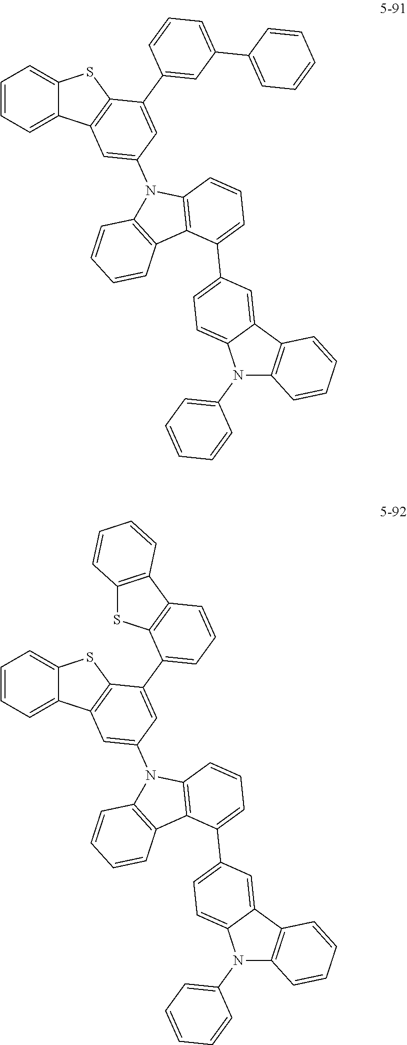 [SMILES: c1ccc(-c2cccc(-c3cc(-n4c5ccccc5c5c(-c6ccc7c(c6)c6ccccc6n7-c6ccccc6)cccc54)cc4c3sc3ccccc34)c2)cc1 ; c1ccc(-n2c3ccccc3c3cc(-c4cccc5c4c4ccccc4n5-c4cc(-c5cccc6c5sc5ccccc56)c5sc6ccccc6c5c4)ccc32)cc1]

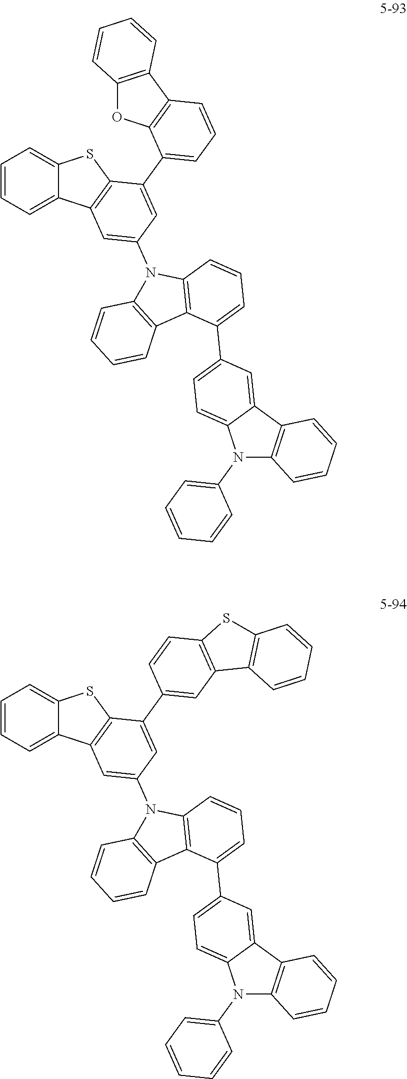 [SMILES: c1ccc(-n2c3ccccc3c3cc(-c4cccc5c4c4ccccc4n5-c4cc(-c5cccc6c5oc5ccccc56)c5sc6ccccc6c5c4)ccc32)cc1 ; c1ccc(-n2c3ccccc3c3cc(-c4cccc5c4c4ccccc4n5-c4cc(-c5ccc6sc7ccccc7c6c5)c5sc6ccccc6c5c4)ccc32)cc1]

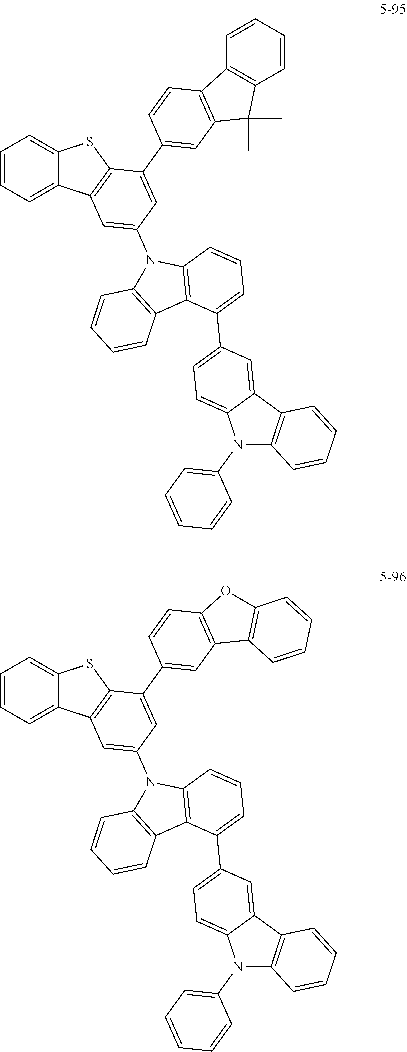 [SMILES: CC1(C)c2ccccc2-c2ccc(-c3cc(-n4c5ccccc5c5c(-c6ccc7c(c6)c6ccccc6n7-c6ccccc6)cccc54)cc4c3sc3ccccc34)cc21 ; c1ccc(-n2c3ccccc3c3cc(-c4cccc5c4c4ccccc4n5-c4cc(-c5ccc6oc7ccccc7c6c5)c5sc6ccccc6c5c4)ccc32)cc1]

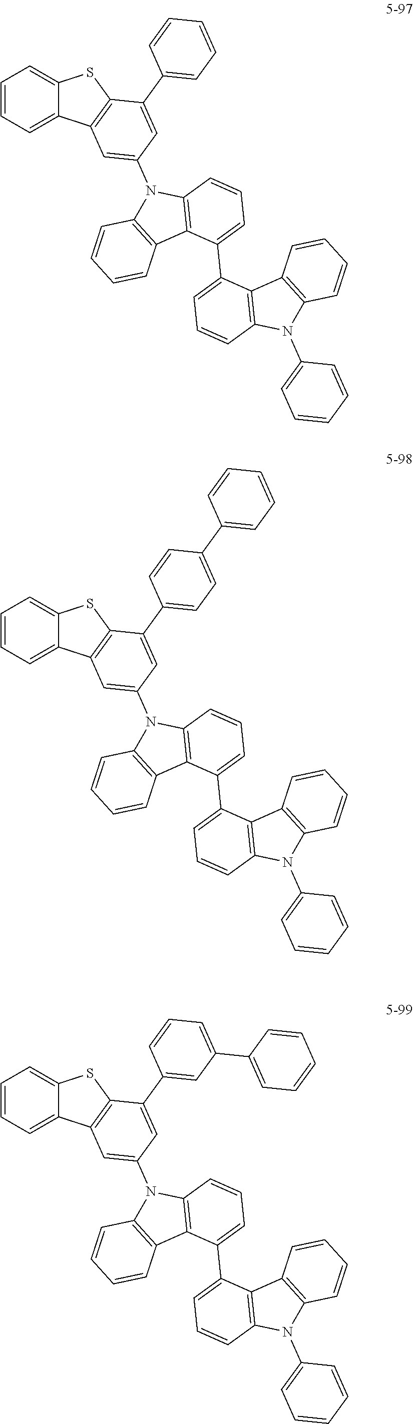 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-c3cc(-n4c5ccccc5c5c(-c6cccc7c6c6ccccc6n7-c6ccccc6)cccc54)cc4c3sc3ccccc34)cc2)cc1 ; c1ccc(-c2cccc(-c3cc(-n4c5ccccc5c5c(-c6cccc7c6c6ccccc6n7-c6ccccc6)cccc54)cc4c3sc3ccccc34)c2)cc1]

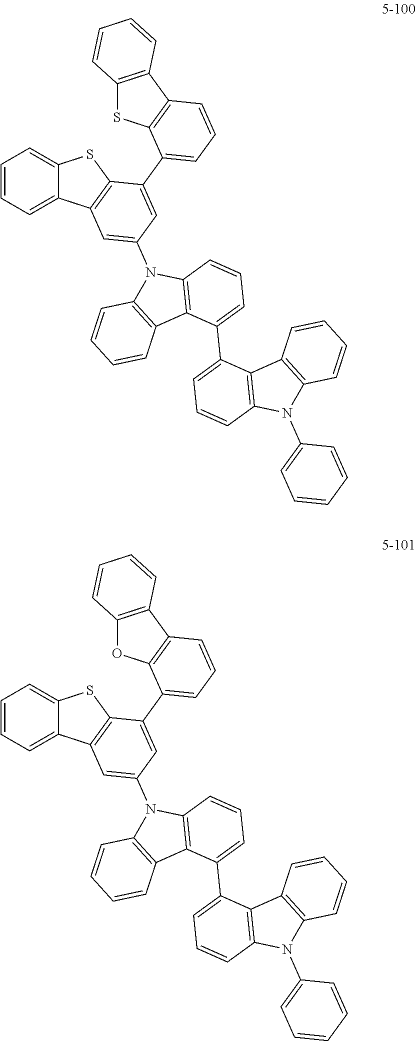 [SMILES: c1ccc(-n2c3ccccc3c3c(-c4cccc5c4c4ccccc4n5-c4cc(-c5cccc6c5sc5ccccc56)c5sc6ccccc6c5c4)cccc32)cc1 ; c1ccc(-n2c3ccccc3c3c(-c4cccc5c4c4ccccc4n5-c4cc(-c5cccc6c5oc5ccccc56)c5sc6ccccc6c5c4)cccc32)cc1]

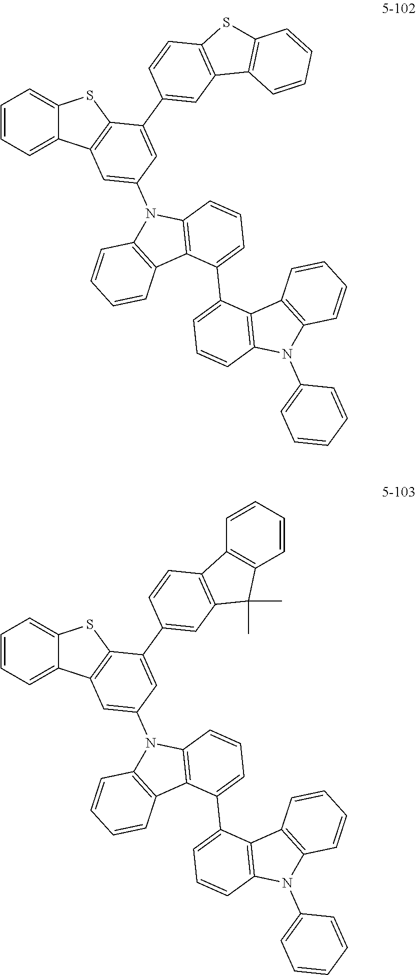 [SMILES: c1ccc(-n2c3ccccc3c3c(-c4cccc5c4c4ccccc4n5-c4cc(-c5ccc6sc7ccccc7c6c5)c5sc6ccccc6c5c4)cccc32)cc1 ; CC1(C)c2ccccc2-c2ccc(-c3cc(-n4c5ccccc5c5c(-c6cccc7c6c6ccccc6n7-c6ccccc6)cccc54)cc4c3sc3ccccc34)cc21]

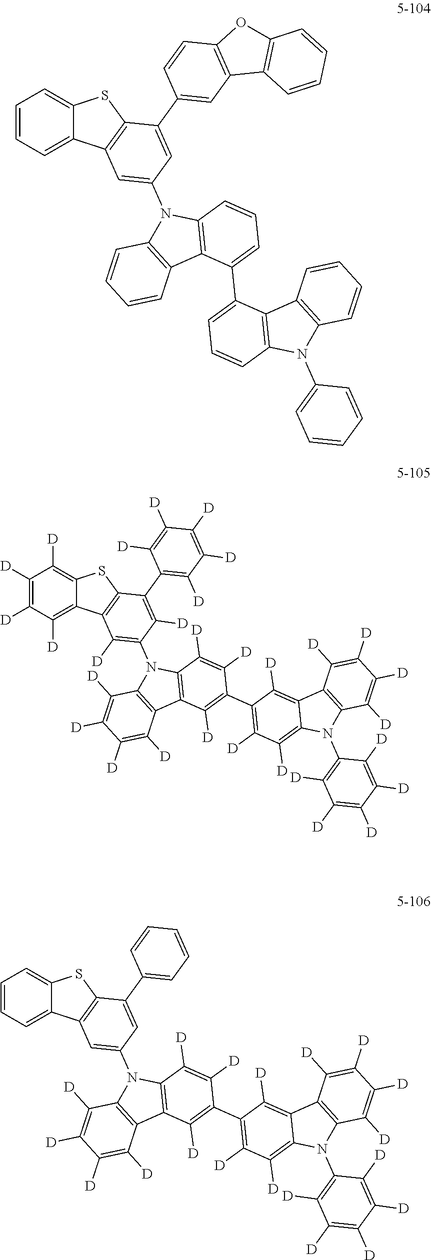

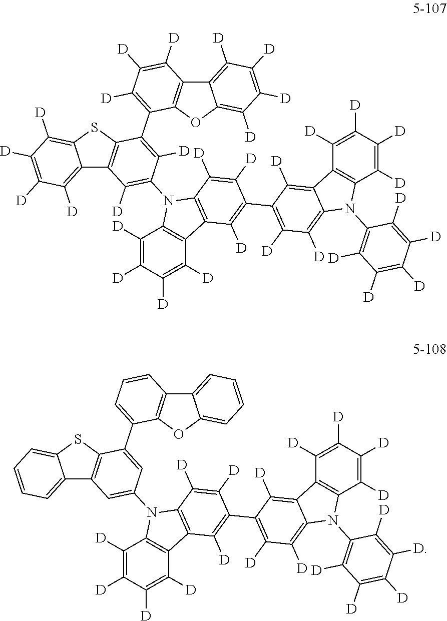

15. The organic light emitting device of claim 11, wherein Chemical Formula 14 is represented by any one of the following compounds:

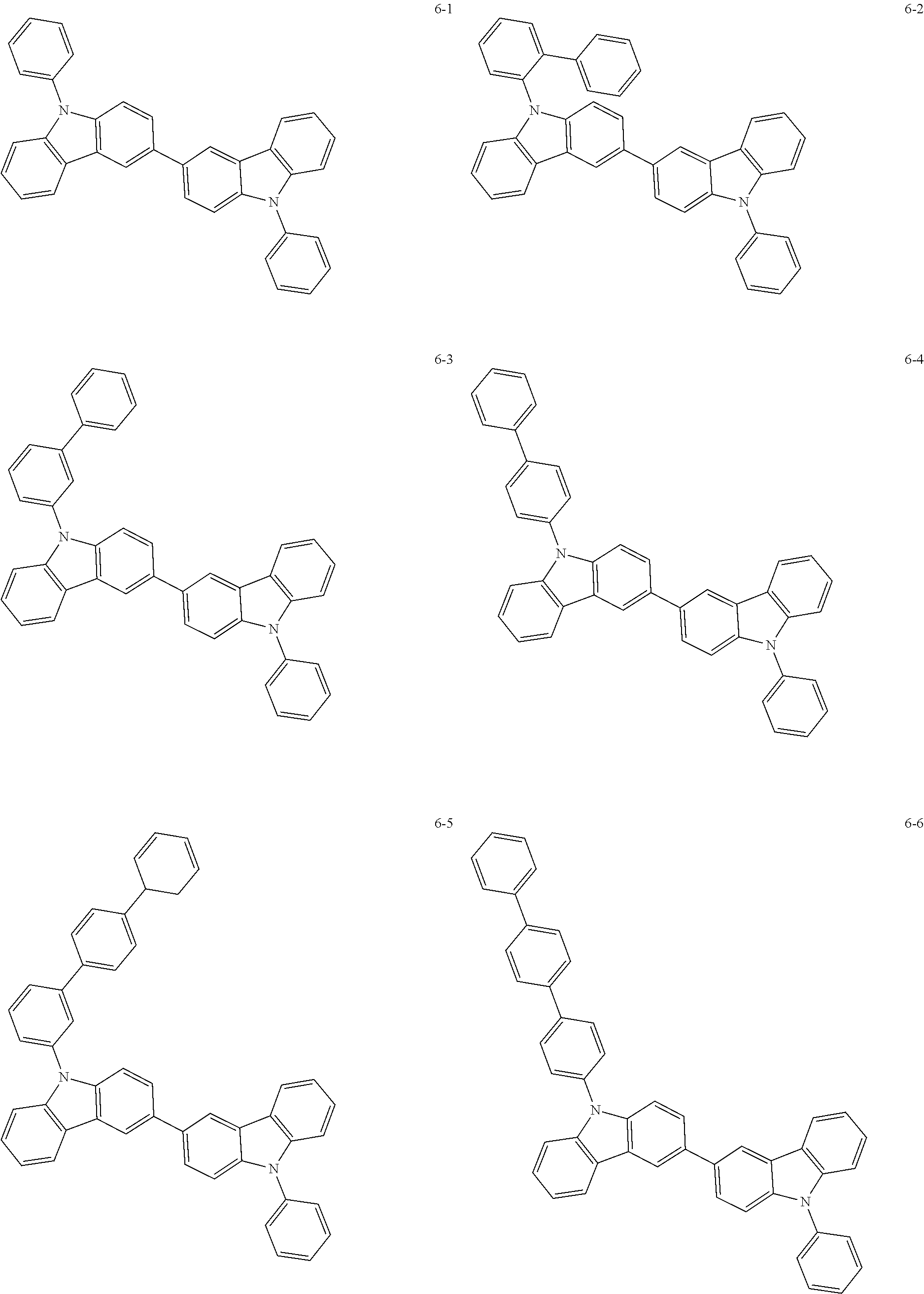 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; C1=CCC(c2ccc(-c3cccc(-n4c5ccccc5c5cc(-c6ccc7c(c6)c6ccccc6n7-c6ccccc6)ccc54)c3)cc2)C=C1 ; c1ccc(-c2ccc(-c3ccc(-n4c5ccccc5c5cc(-c6ccc7c(c6)c6ccccc6n7-c6ccccc6)ccc54)cc3)cc2)cc1]

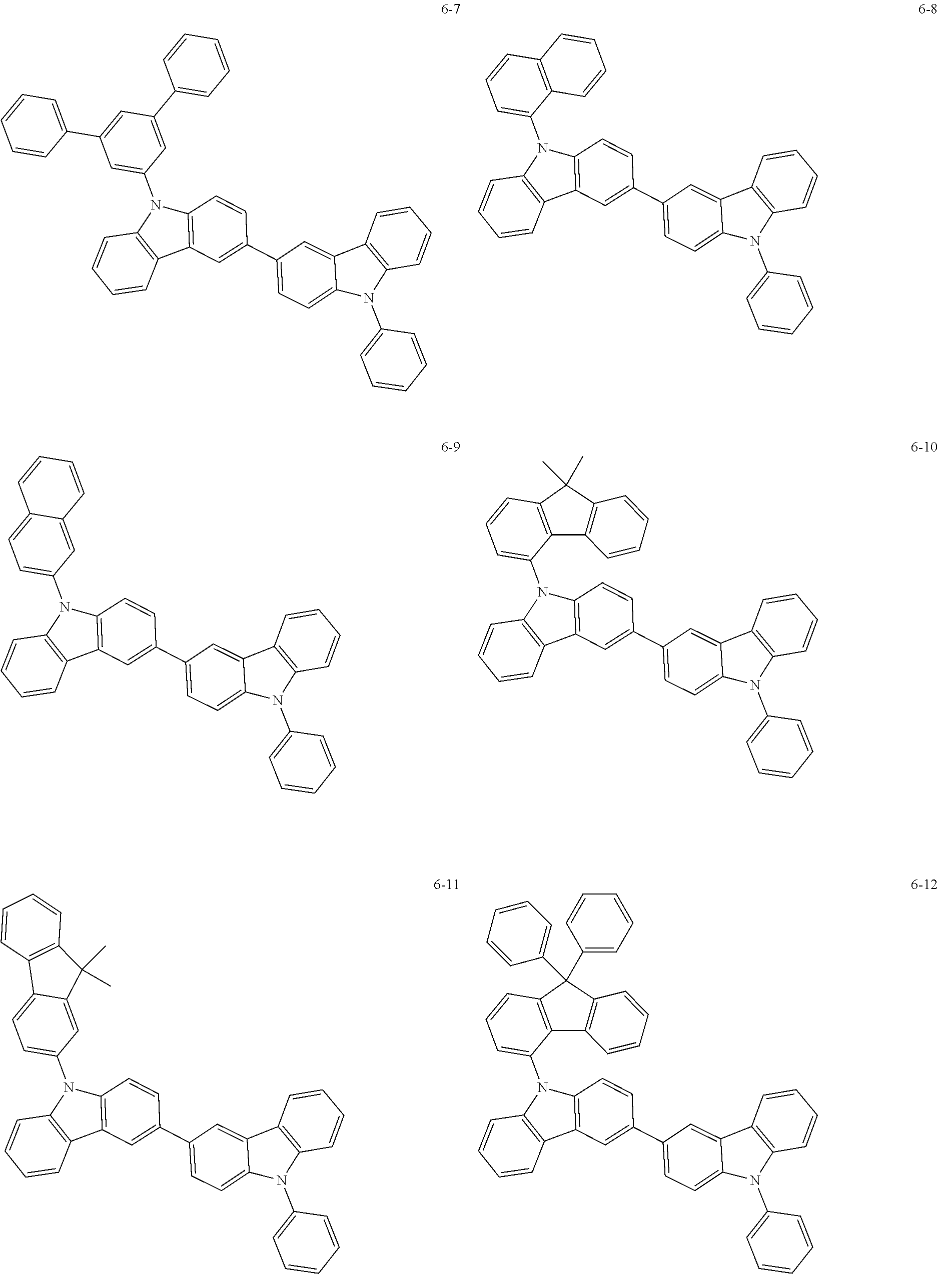 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

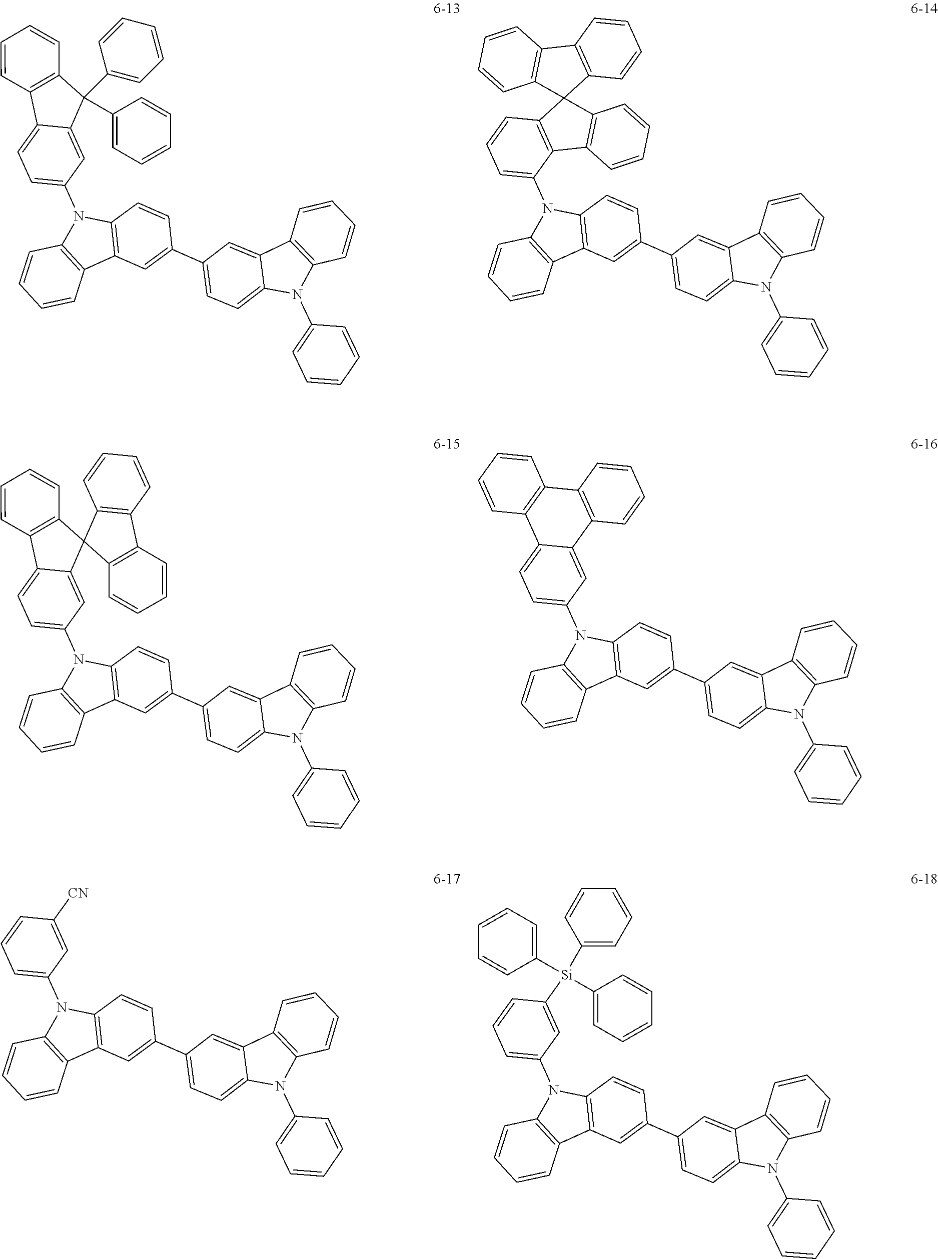

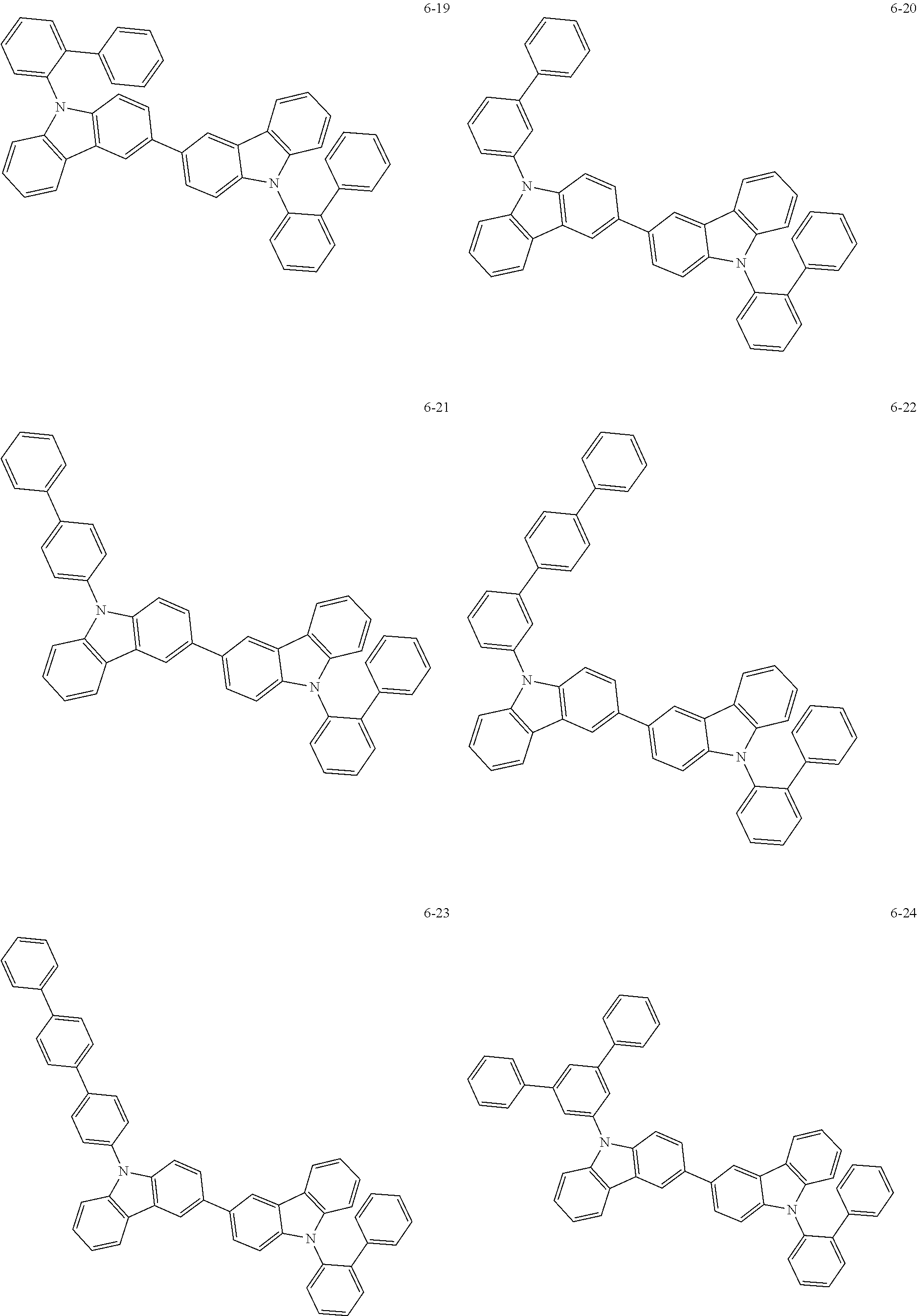

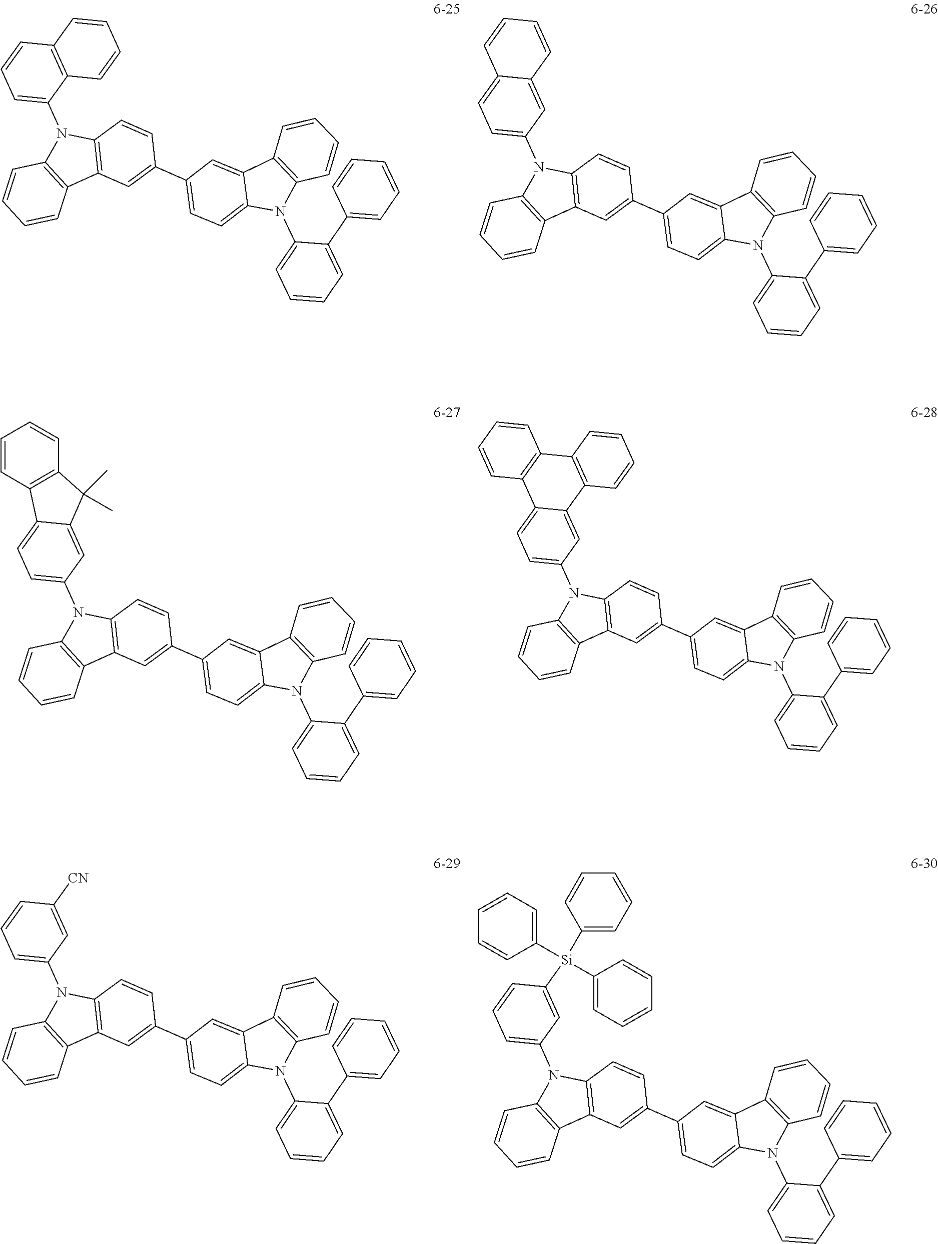

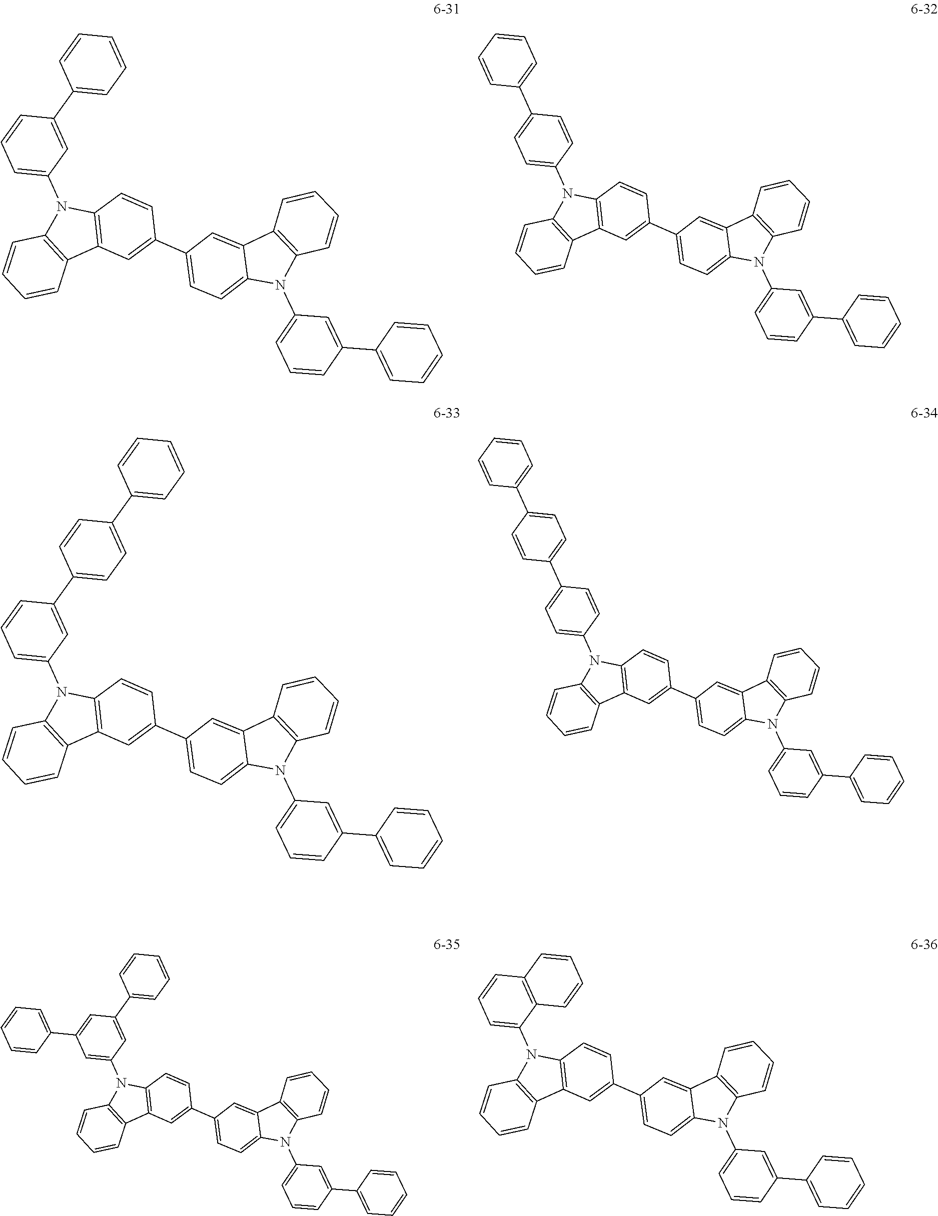

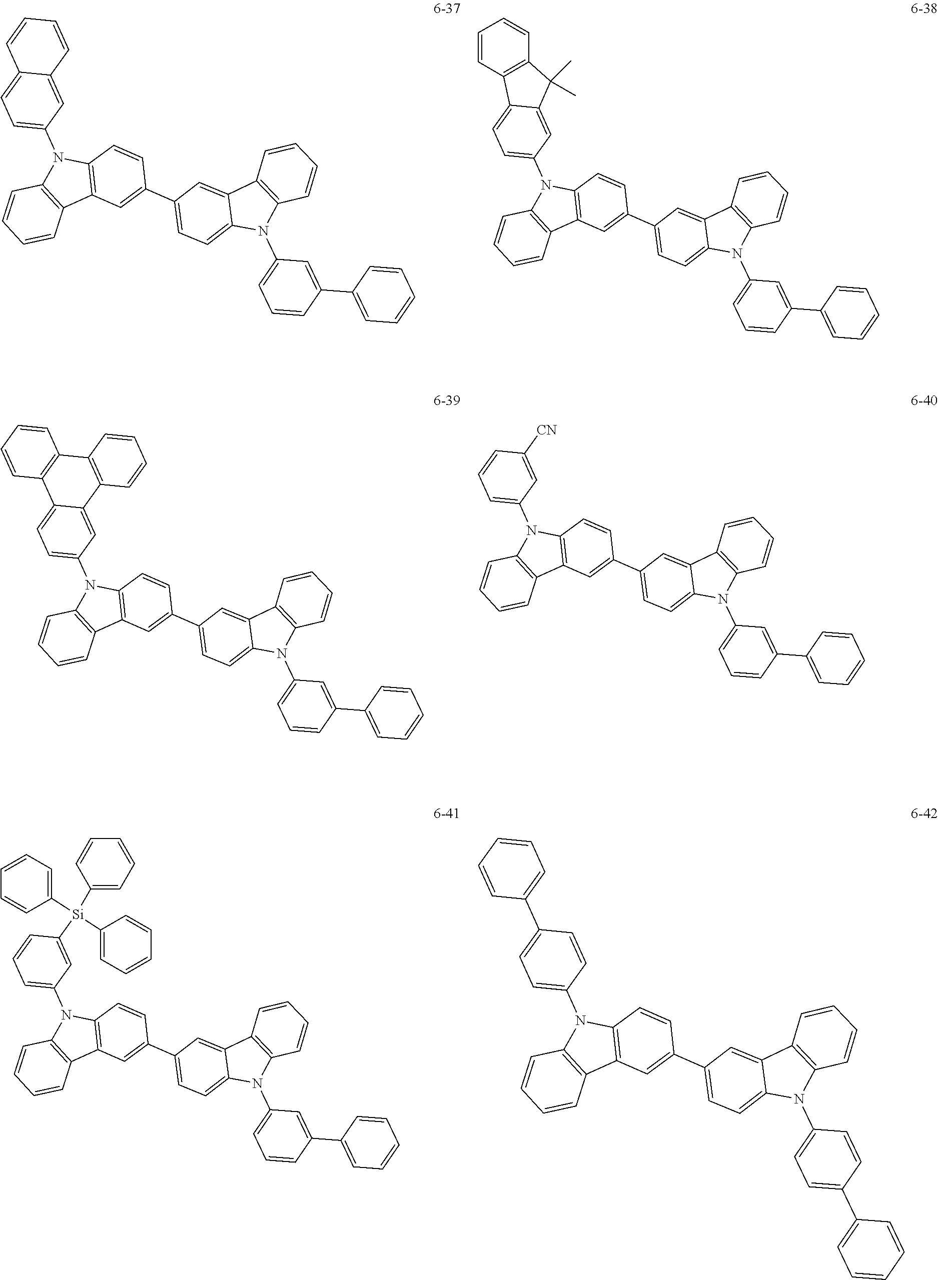

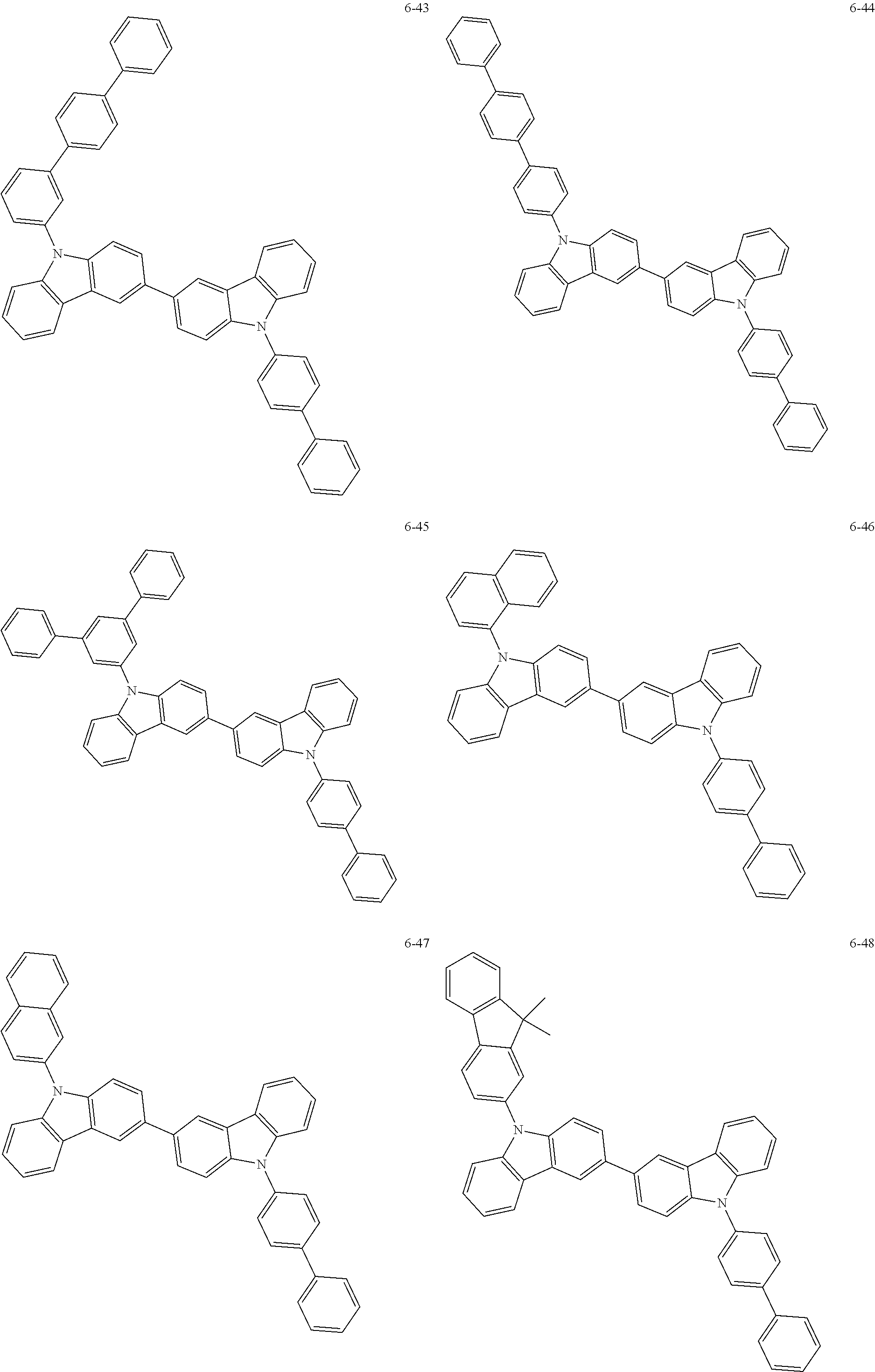

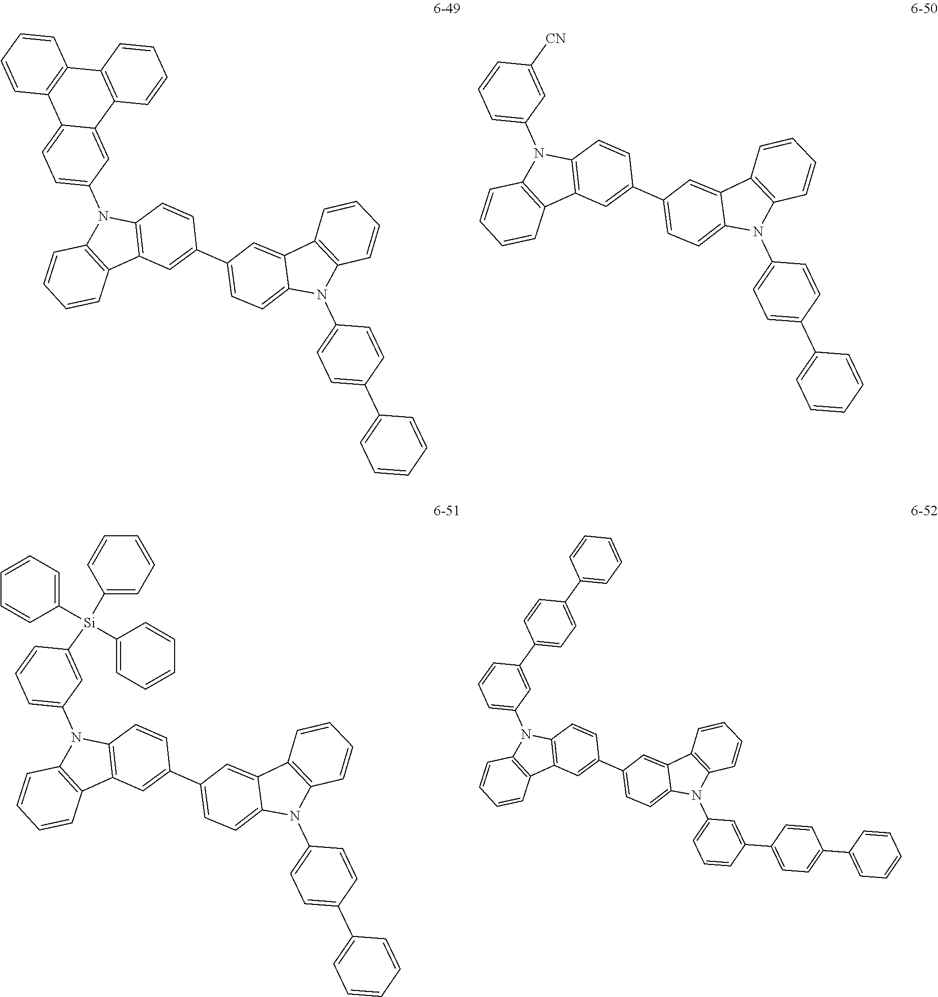 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

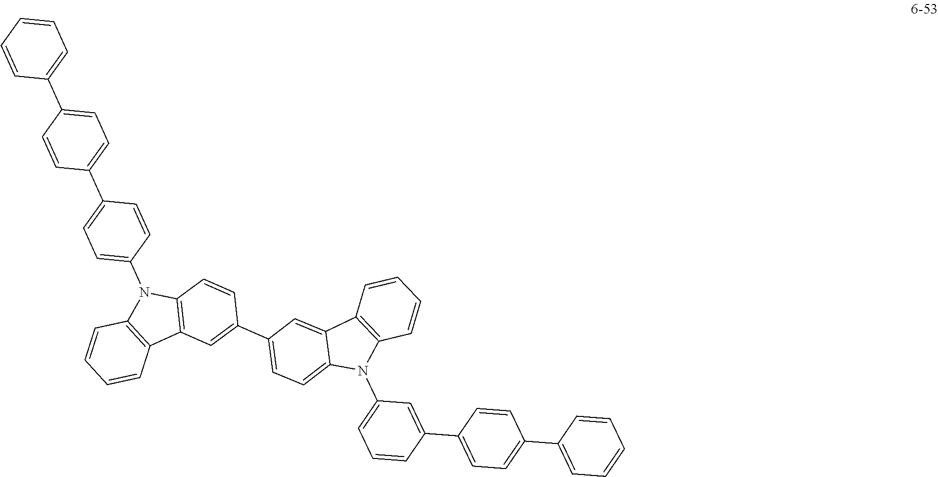 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1]

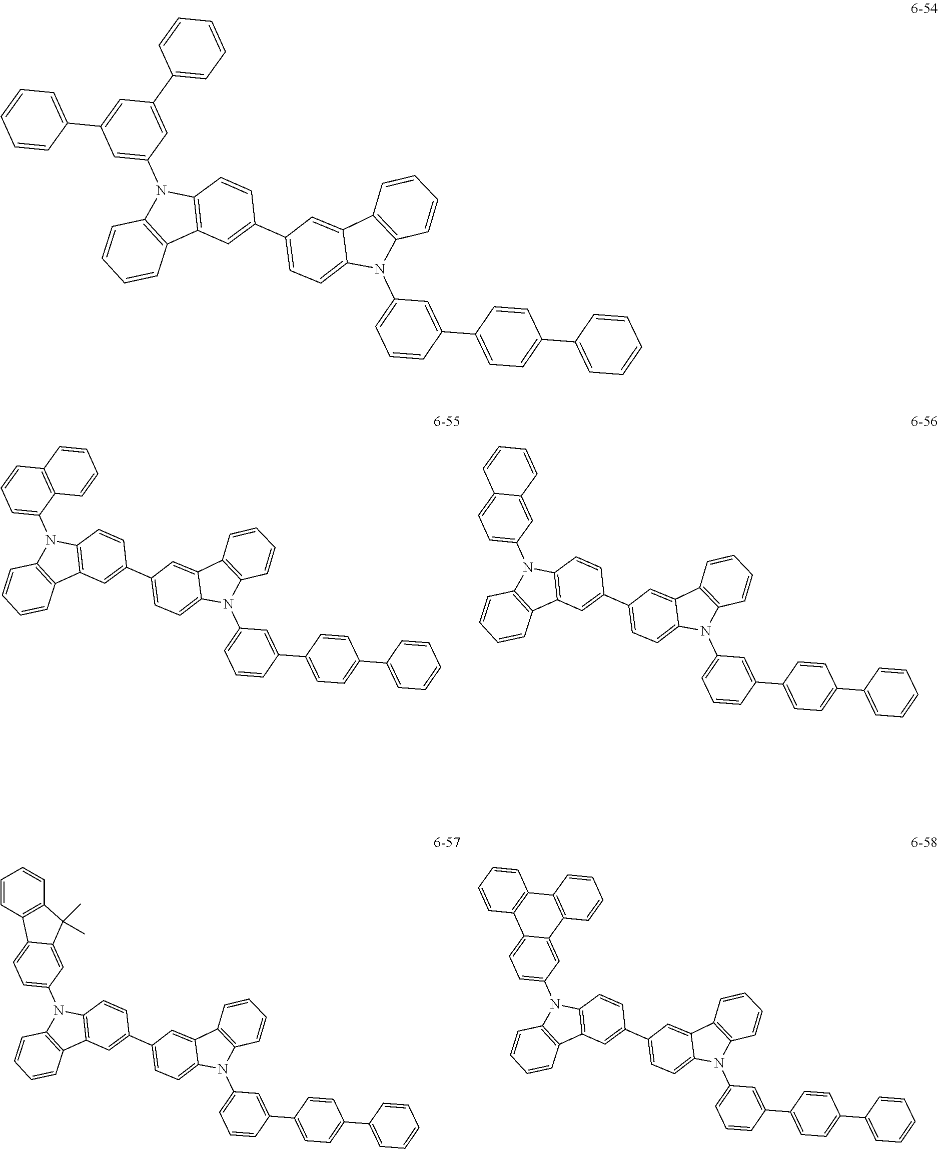

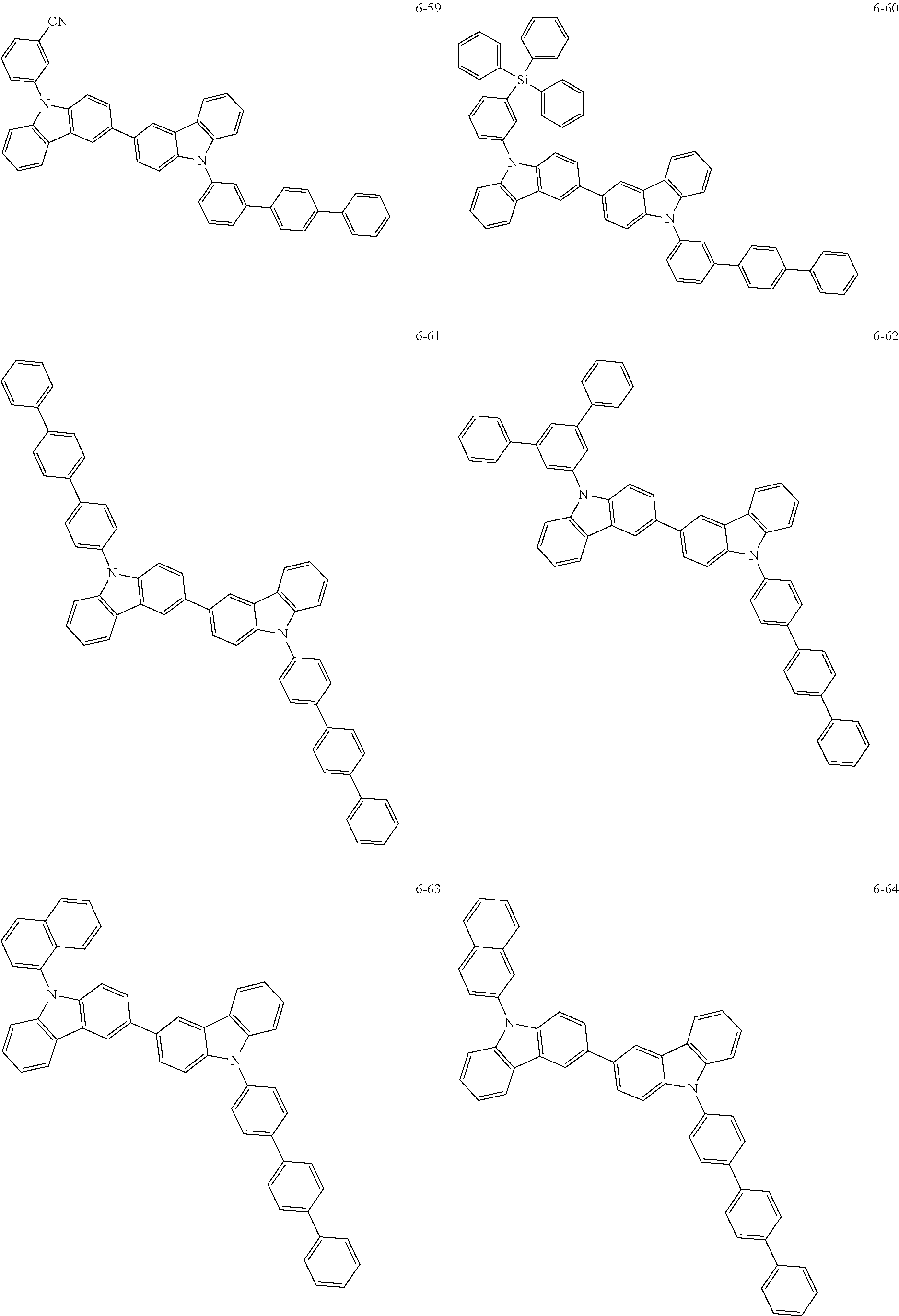

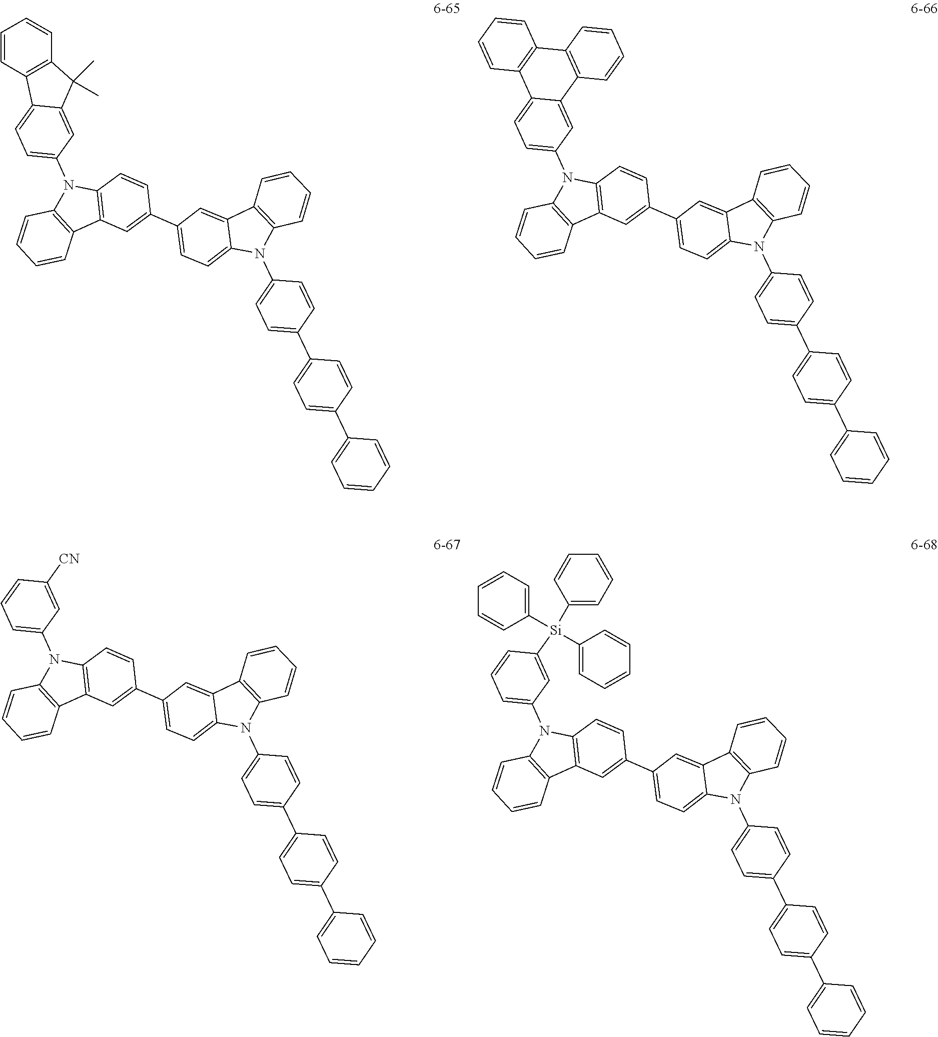 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

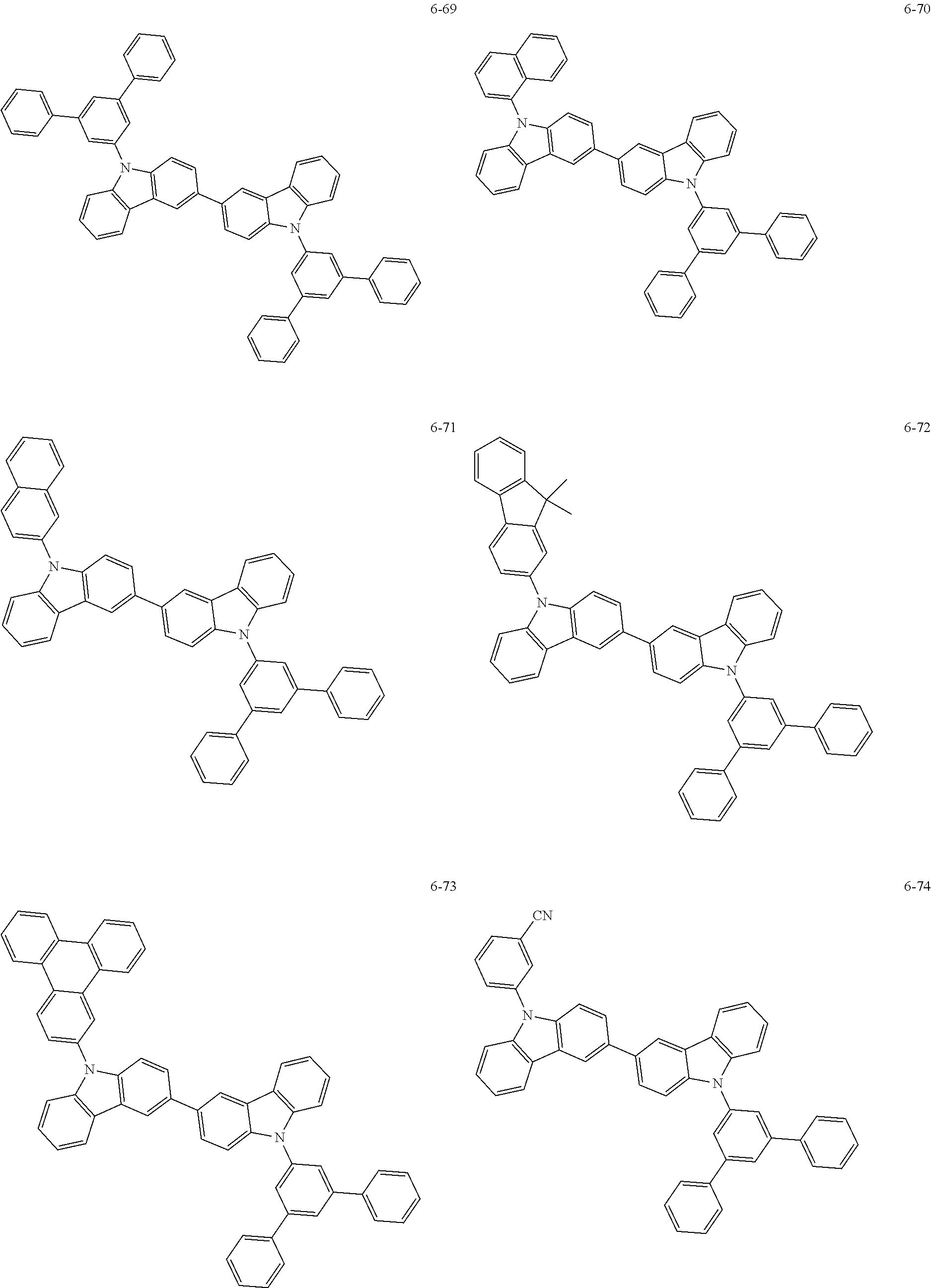

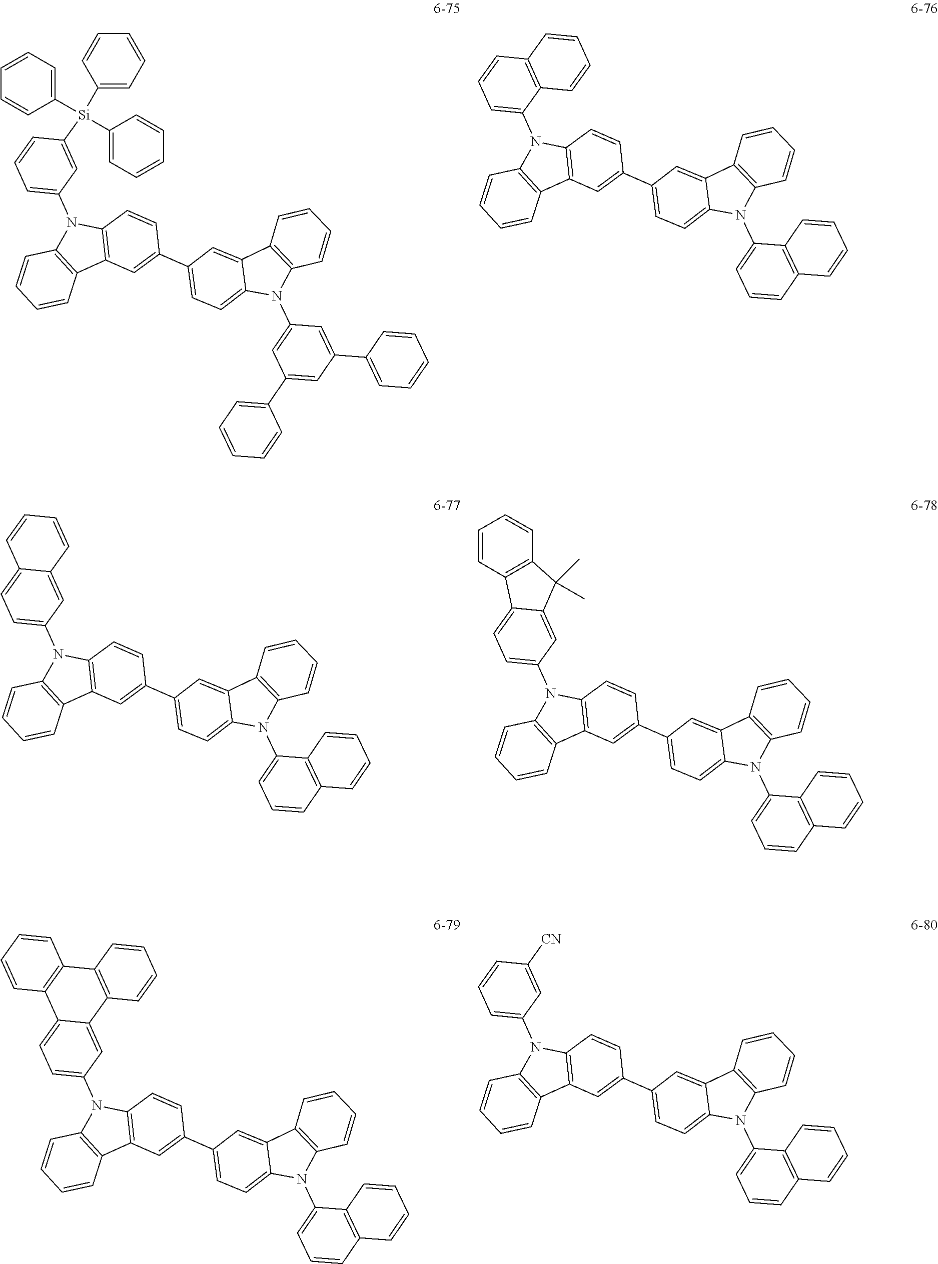

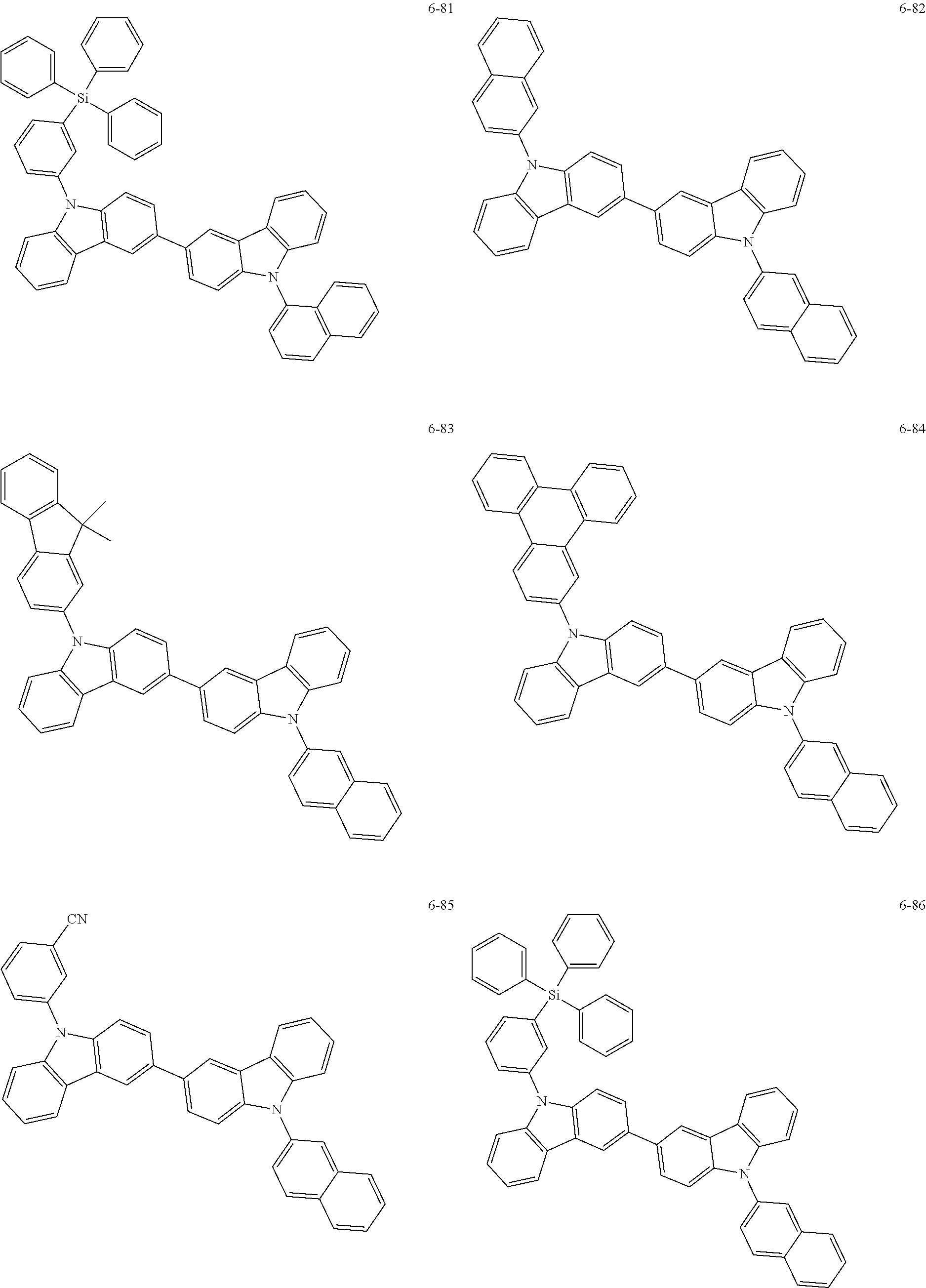

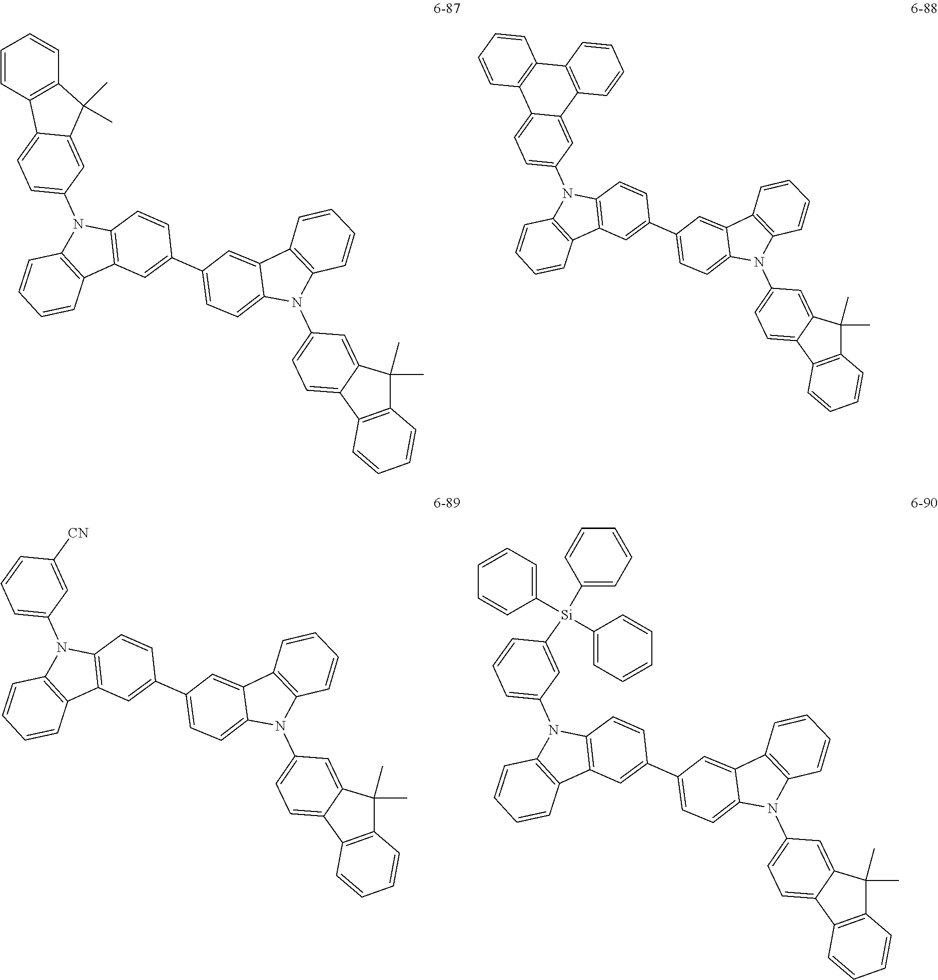 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

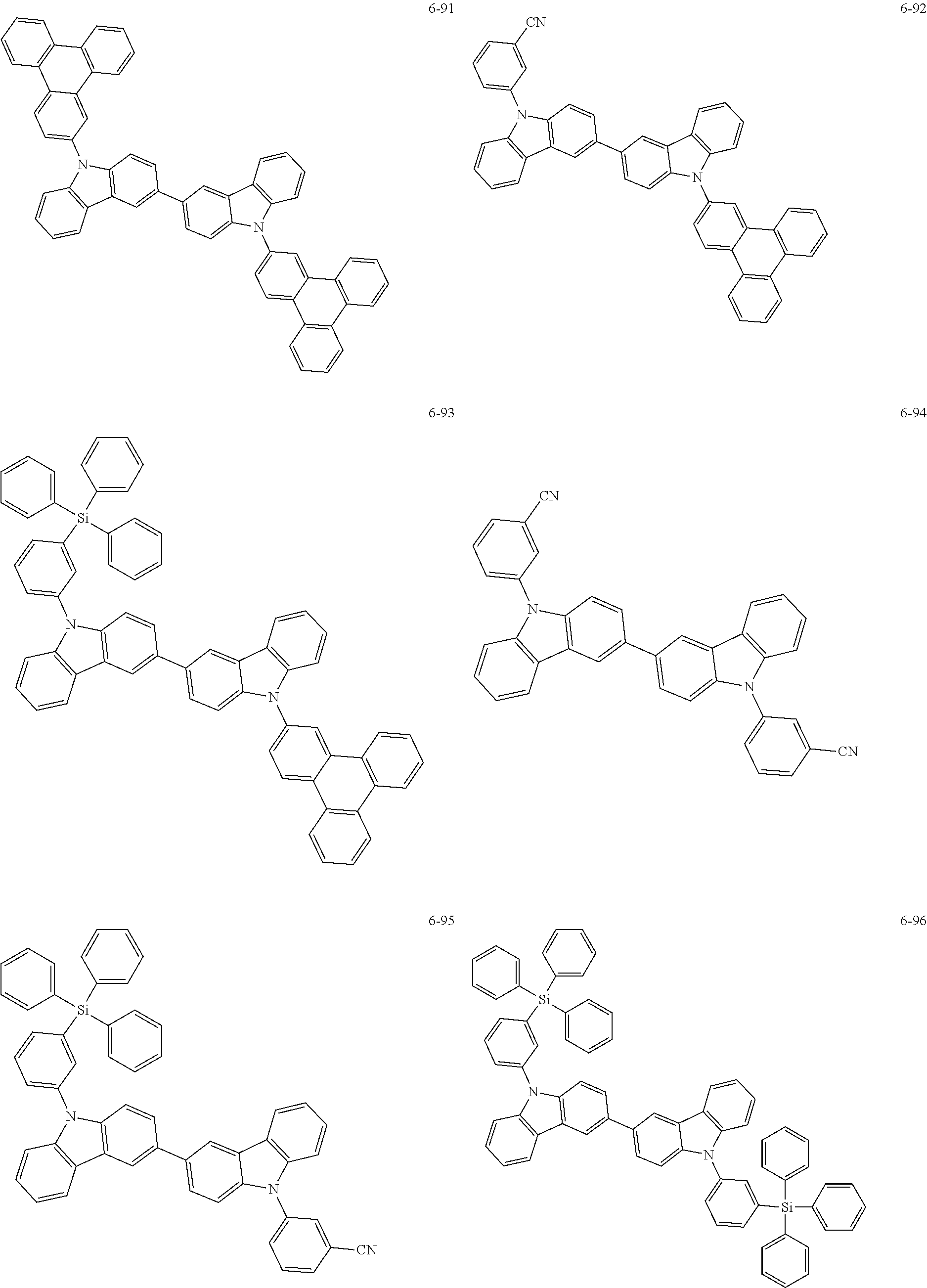

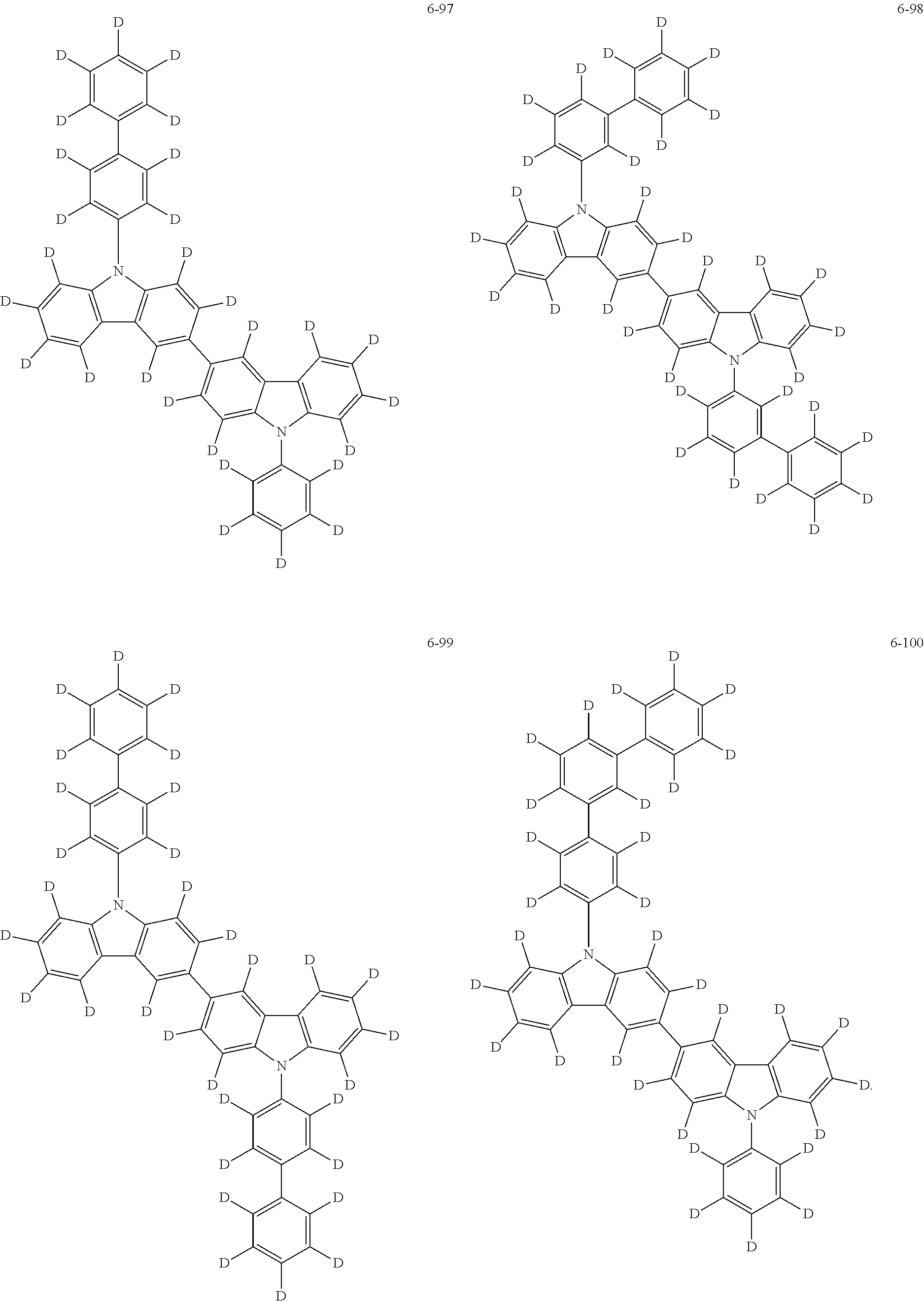

16. A composition for an organic material layer of an organic light emitting device, the composition comprising:
the heterocyclic compound represented by Chemical Formula 1 of claim 1; and
a heterocyclic compound represented by the following Chemical Formula 13 or 14: 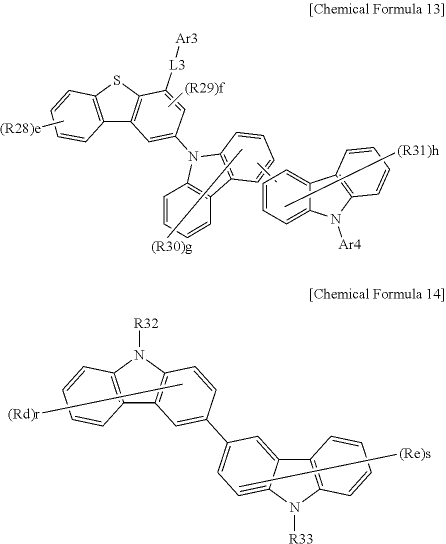
wherein, in Chemical Formulae 13 and 14,
L3 is a direct bond; a substituted or unsubstituted arylene group having 6 to 60 carbon atoms; or a substituted or unsubstituted heteroarylene group having 2 to 60 carbon atoms;
R28 to R31, Rd and Re are the same as or different from each other, and each independently selected from the group consisting of hydrogen; deuterium; halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 2 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more groups adjacent to each other bond to each other to form a substituted or unsubstituted aromatic hydrocarbon ring having 6 to 60 carbon atoms or a substituted or unsubstituted heteroring having 2 to 60 carbon atoms, r and s are an integer of 0 to 7, and when r is 2 or greater, Rds are the same as or different from each other, when s is 2 or greater, Res are the same as or different from each other, e is an integer of 0 to 4, g and h are each an integer of 0 to 7, f is an integer of 0 to 2, and when e is 2 or greater, R28s are the same as or different from each other, when f is an integer of 2, R29s are the same as or different from each other, when g is 2 or greater, R30s are the same as or different from each other, and when h is 2 or greater, R31s are the same as or different from each other;
Ar3 is a substituted or unsubstituted aryl group having 6 to 60 carbon atoms; or a substituted or unsubstituted heteroaryl group having 2 to 60 carbon atoms;
Ar4 is a substituted or unsubstituted aryl group having 6 to 60 carbon atoms; or a substituted or unsubstituted heteroaryl group having 2 to 60 carbon atoms; and
R32 and R33 are the same as or different from each other, and each independently a substituted or unsubstituted silyl group; a substituted or unsubstituted aryl group having 6 to 60 carbon atoms; or a substituted or unsubstituted heteroaryl group having 2 to 60 carbon atoms.

17. The composition for an organic material layer of an organic light emitting device of claim 16, wherein, in the composition, the heterocyclic compound represented by Chemical Formula 1:the heterocyclic compound represented by Chemical Formula 13 or 14 have a weight ratio of 1:10 to 10:1.
